(19) United States
(12) Patent Application Publication
(10) Pub. No.: US 2023/0158151 A1
(43) Pub. Date: May 25, 2023

(54) COMPOUNDS FOR INHIBITING OR DEGRADING TARGET PROTEINS, COMPOSITIONS, COMPRISING THE SAME, METHODS OF THEIR MAKING, AND METHODS OF THEIR USE

(71) Applicant: NURIX THERAPEUTICS, INC., San Francisco, CA (US)

(72) Inventors: Eric Wegryzniak, San Francisco, CA (US); Joel McIntosh, San Francisco, CA (US); Daisuke Kato, San Francisco, CA (US); Jeffrey Mihalic, San Francisco, CA (US); Ge Peng, San Francisco, CA (US)

(21) Appl. No.: 17/889,062

(22) Filed: Aug. 16, 2022

Related U.S. Application Data
(63) Continuation of application No. PCT/US22/27512, filed on May 3, 2022.
(60) Provisional application No. 63/304,497, filed on Jan. 28, 2022.
(60) Provisional application No. 63/183,619, filed on May 3, 2021.

(51) Int. Cl. A61K 47/54

(52) U.S. Cl. CPC A61K 47/545

(57) ABSTRACT

Provided herein are heterobifunctional compounds which find utility as modulators of targeted ubiquitination. Also provided herein are pharmaceutically acceptable compositions comprising said compounds and methods of using the compounds, and compositions in the treatment of various diseases, conditions, or disorders.

### CROSS-REFERENCE

This application claims the benefit of U.S. provisional application No. 63/304,497, filed Jan. 28, 2022, and U.S. provisional application No. 63/183,619, filed, May 3, 2021, the contents of which are hereby incorporated by reference in their entireties.

### FIELD

This disclosure provides heterobifunctional compounds for proteolytically degrading a target protein and methods for treating diseases modulated by the target protein.

### BACKGROUND

Degradation of cellular proteins is required for normal maintenance of cellular function, including proliferation, differentiation, and cell death. The irreversible nature of proteolysis makes it well-suited to serve as a regulatory switch for controlling unidirectional processes. This principle is evident in the control of the cell cycle, where initiation of DNA replication, chromosome segregation, and exit from mitosis are triggered by the destruction of key regulatory proteins.

In eukaryotes, protein degradation is predominately mediated through the ubiquitin pathway in which proteins targeted for destruction are ligated to the polypeptide ubiquitin. The process of protein ubiquitination proceeds through an enzymatic cascade that consists of a ubiquitin activating enzyme (E1), ubiquitin conjugating enzyme (E2), and an E3 ubiquitin ligase (E3). E3 ligases are diverse proteins. They selectively modify proteins by covalently attaching ubiquitin to lysine, serine, threonine, or cysteine residues within each particular E3's substrate.

Modulation of E3 ligases as therapeutic agents can be more attractive than general proteasome inhibitors, due to their specificity for certain protein substrates. Specific degradation of a target protein could be accomplished using heterobifunctional small molecules to recruit a target protein to a ubiquitin ligase, thus promoting ubiquitination and proteasomal degradation of the target protein.

### SUMMARY

Provided herein are heterobifunctional compounds of Formula (I) and, compositions comprising the compounds, methods of producing the compounds, and methods of using the compounds and compositions in treatment of a disease or disorder mediated by a target protein. In certain embodiments, the compound degrade the target protein via a ubiquitin proteolysis pathway.

In one aspect, provided herein is a compounds of Formula (I), or a stereoisomer and/or a pharmaceutically acceptable salt thereof:

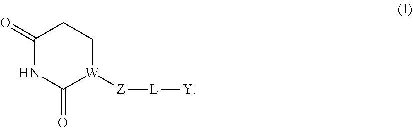

The left side of the molecule is a ubiquitin ligase harness. The right side of the molecule (Y moiety) is capable of binding to a target protein. The middle portion of the molecule (L moiety) is a linker. W is —CH or —N. Z is selected from the group consisting of:

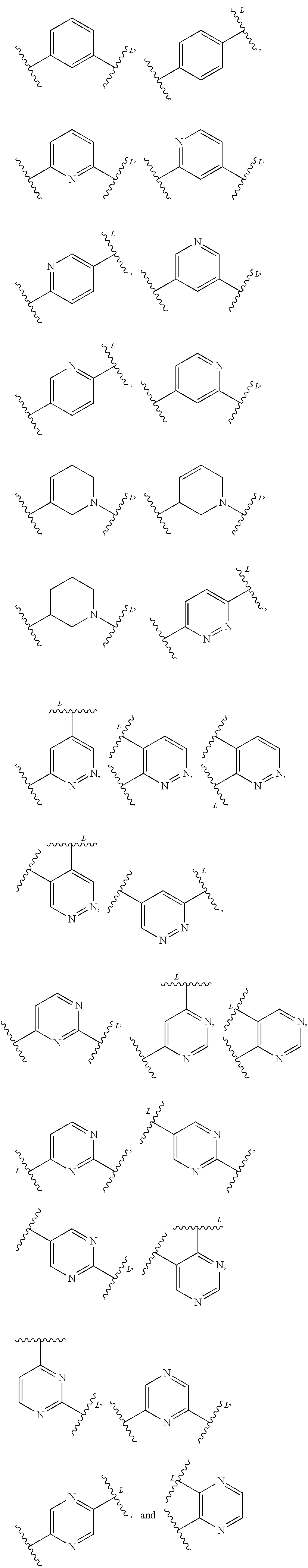

The compounds are described in detail herein.

In one aspect, provided herein is a compounds of Formula (I), or a stereoisomer and/or a pharmaceutically acceptable salt thereof:

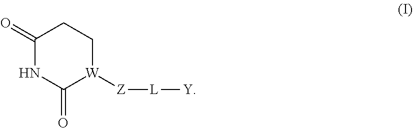

The left side of the molecule is a ubiquitin ligase harness. The right side of the molecule (Y moiety) is capable of binding to a target protein. The middle portion of the molecule (L moiety) is a linker. W is —CH or —N. Z is selected from the group consisting of:

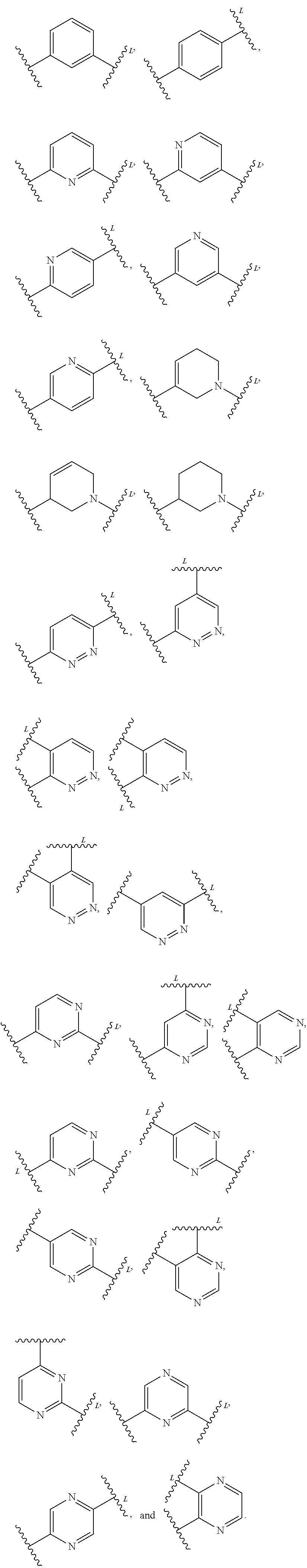

The compounds are described in detail herein.

In another aspect, provided herein are compositions comprising the compound of Formula (I). In some embodiments, the compositions are pharmaceutical compositions. The pharmaceutical compositions comprise the compound and any suitable pharmaceutically acceptable carrier, excipient, or diluent.

In another aspect, provided herein are methods of making degrader compounds. The methods comprise the step of linking Y, a target binding moiety, which binds to a target protein, to a degron, directly or via a linker L, to form the degrader compound. In certain embodiments, the target binding moiety Y is a moiety described herein. In certain embodiments, the degron is a harness described herein. In certain embodiments, the linker L is a linker described herein. The degron can be linked to L then linked to Y, or Y can be linked to L then linked to the degron. In certain embodiments, the methods further comprise the step of contacting a target protein with the degrader compound.

In another aspect, provided herein are methods of using the compounds or compositions described herein. In some embodiments, the methods are for the treatment of a disease or disorder mediated by a target protein in a patient in need thereof. In some embodiments, the target protein is Bruton's tyrosine kinase (BTK). In some embodiments, the target protein is tyrosine-protein kinase (ITK/TSK). In some embodiments, the target protein is bromodomain-containing protein 4 (BRD4). In some embodiments, the target protein is FMS-like tyrosine kinase 3 (FLT-3). In some embodiments, the target protein is Brg/Brahma-associated factors (BAF complex). In some embodiments, the target protein is induced myeloid leukemia cell differentiation protein (MCL-1). In some embodiments, the target protein is signal transducer and activator of transcription 3 (STAT3). In some embodiments, the target protein is barrier-to-autointegration factor (BAF). In some embodiments, the target protein is BCR-ABL. In some embodiments, the disease is cancer. In some embodiments, the disease is an autoimmune disease. In certain embodiments, the target protein is selected from the group consisting of CDK4, CDK6, SHP-2, FGFR1, FGFR3, FGFR1 fusions, FGFR3 fusions, MDM2, TRIM24, SARS-COV2 proteins (e.g. Mpro, or nsp5), PI3K, PI3K delta, MEK, BCR-ABL, MLL, MALT1, IRAK1, IRAK4, and kinases (e.g., broad spectrum). In some embodiments, the disease is an autoimmune disease. In certain embodiments, the target protein is selected from the group consisting of CDK4, CDK6, SHP-2, FGFR1, FGFR3, FGFR1 fusions, FGFR3 fusions, MDM2, TRIM24, SARS-COV2 proteins (e.g. Mpro, or nsp5), PI3K, PI3K delta, MEK, BCR-ABL, MLL, and MALT1. In certain embodiments, the target protein is SH2 domain-containing protein tyrosine phosphatase-2 (SHP-2). In certain embodiments, the target protein is fibroblast growth factor receptor (FGFR). In certain embodiments, the target protein is FGFR1 fusion. In certain embodiments, the target protein is FGFR3 fusion. In certain embodiments, the target protein is mouse double minute 2 homolog (MDM2). In certain embodiments, the target protein is tripartite motif containing 24 (TRIM24). In certain embodiments, the target protein is SARS-COV2 main protease (Mpro). In certain embodiments, the target protein is phosphoinositide 3-kinase (PI3K) delta. In certain embodiments, the target protein is mitogen-activated protein kinase kinase (MEK). In certain embodiments, the target protein is histone-lysine N-methyltransferase 2A (MLL). In certain embodiments, the target protein is mucosa-associated lymphoid tissue lymphoma translocation protein 1 (MALT1). In certain embodiments, the target protein is Interleukin 1 Receptor Associated Kinase 1 (IRAK1). In certain embodiments, the target protein is one or more kinases. In certain embodiments, the target proteins are a plurality of kinases.

In one aspect, provided herein are methods of treating or preventing cancer in a subject in need thereof. In certain embodiments, the methods comprise the step of orally administering to the subject an amount of a heterobifunctional compound capable of inducing proteolytic degradation of a target protein, wherein said target protein is selected from the group consisting of BTK; ITK/TSK; BRD4; FLT-3; BAF; BAF complex; MCL-1; STAT3; and BCR-ABL. In certain embodiments, the methods comprise the step of orally administering to the subject an amount of a heterobifunctional compound capable of inducing proteolytic degradation of a target protein, wherein said target protein is selected from the group consisting of CDK4, CDK6, SHP-2, FGFR1, FGFR3, FGFR1 fusions, FGFR3 fusions, MDM2, TRIM24, SARS-COV2 proteins (e.g. Mpro, or nsp5), PI3K, PI3K delta, MEK, BCR-ABL, MLL, and MALT1. In certain embodiments, the methods comprise the step of orally administering to the subject an amount of a heterobifunctional compound capable of inducing proteolytic degradation of a target protein, wherein said target protein is selected from the group consisting of CDK4, CDK6, SHP-2, FGFR1, FGFR3, FGFR1 fusions, FGFR3 fusions, MDM2, TRIM24, SARS-COV2 proteins (e.g. Mpro, or nsp5), PI3K, PI3K delta, MEK, BCR-ABL, MLL, MALT1, IRAK1, and kinases (e.g., broad spectrum). In certain embodiments, the amount is effective to treat or prevent the cancer.

Also provided herein are uses of compounds described herein, and compositions thereof, for the treatment of cancer. Also provided herein are uses of compounds described herein, and compositions thereof, for the treatment of autoimmune diseases and inflammatory diseases.

### BRIEF DESCRIPTION OF THE FIGURES

FIG. 1 provides in vitro degradation of ITK in a HiBiT cell line.

FIG. 2A provides in vivo degradation of ITK in mouse splenocytes following administration of compounds 30 and 31 provided herein observed by Western blotting; FIG. 2B provides graphs of ITK levels in mouse splenocytes following administration of compounds 30 and 32 provided herein.

FIG. 3A provides in vivo degradation of ITK in mouse splenocytes following administration of compounds 30 and 29 provided herein observed by Western blotting; FIG. 3B provides graphs of ITK levels in mouse splenocytes following administration of compounds 30 and 29 provided herein; FIG. 3C provides compound 30 concentration over time in vivo.

### DETAILED DESCRIPTION OF THE PREFERRED EMBODIMENTS

Provided herein are heterobifunctional compounds that induce the proteolytic degradation of a target protein via a ubiquitin proteolysis pathway. Also provided herein are compounds of Formula (I-XXIX), (Ia-If), (IIa-IIw), or (I1-I115) and a stereoisomer and/or a pharmaceutically acceptable salt thereof.

As used herein, the following definitions shall apply unless otherwise indicated.

### Definitions

As used herein, the term “hydroxyl” or “hydroxy” refers to an —OH moiety.

As used herein, the term “aliphatic” encompasses the terms alkyl, alkenyl, and alkynyl, each of which are optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonyl amino, (heterocycloalkylalkyl)carbonylamino, heteroarylcarbonylamino, heteroaralkylcarbonylamino, alkyl aminocarbonyl, cycloalkylaminocarbonyl, heterocycloalkyl aminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-4 or 2-6) carbon atoms and at least one double bond. Like an alkyl group, an alkenyl group can be straight or branched. Examples of an alkenyl group include, but are not limited to, allyl, 1- or 2-isopropenyl, 2-butenyl, and 2-hexenyl. An alkenyl group can be optionally substituted with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkyl alkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-4 or 2-6) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino, or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino.” These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl, or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen (H or —H), aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-SO₂— or amino-SO₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl (e.g., mono-, di- (such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkyl sulfonyl)amino)aryl or ((dialkyl)amino)aryl); (cyanoalkyl)aryl; (alkoxy)aryl; (sulfamoyl)aryl (e.g., (aminosulfonyl)aryl); (alkyl sulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkyl sulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl), cycloaliphatic (e.g., cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonyl amino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9-, 10-, or 11-) membered structures that form two rings, wherein the two rings have at least one atom in common (e.g., two atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which are optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino), nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl (e.g., alkyl-SO₂— and aryl-SO₂—), sulfinyl (e.g., alkyl-S(O)—), sulfanyl (e.g., alkyl-S—), sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused, bridged, or spiro) (e.g., 5- to 10-membered mono- or bicyclic) saturated ring structure, in which one or more of the ring atoms is a heteroatom (e.g., nitrogen (N), oxygen (O), sulfur (S), or combinations thereof). Non-limiting examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholinyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, decahydro-2,7-naphthyridine, 2,8-diazaspiro[4.5]decane, 2,7-diazaspiro[3.5]nonane, octahydropyrrolo[3,4-c]pyrrole, octahydro-1H-pyrrolo[3,4-b]pyridine, and 2,6-dioxa-tricyclo[3.3.1.0^{3,7}]nonyl. A monocyclic heterocycloalkyl group can be fused with a phenyl moiety to form structures, such as tetrahydroisoquinoline, that would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring system having four to fifteen ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having two to three rings. For example, a benzofused group includes benzo fused with one or two 4- to 8-membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine.

Without limitation, monocyclic heteroaryls include furyl, thiophene-yl, 2H-pyrrolyl, oxazolyl, thiazolyl, imidazolyl, pyrazolyl, isoxazolyl, isothiazolyl, 1,3,4-thiadiazolyl, 2H-pyranyl, 4H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphaticsulfonyl or aminosulfonyl); sulfinyl (e.g., aliphaticsulfinyl); sulfanyl (e.g., aliphaticsulfanyl); nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkyl sulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkyl sulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkyl sulfonyl)alkyl)heteroaryl; (cyanoalkyl)heteroaryl; (acyl)heteroaryl (e.g., (alkylcarbonyl)heteroaryl); (alkyl)heteroaryl; or (haloalkyl)heteroaryl (e.g., trihaloalkylheteroaryl).

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined herein.

As used herein, an “alkoxy” group refers to an alkyl-O— group where “alkyl” has been defined previously herein.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, when used as a terminal group; or —OC(O)—, or —C(O)O— when used as an internal group.

As used herein, an ester refers to —COOR^{X} when used as a terminal group; or —COOR^{X}— when used as an internal group, wherein R^{X} has been defined above.

As used herein, a formate refers to —OC(O)H.

As used herein, an acetate refers to —OC(O)R^{X}, wherein R^{X} has been defined above.

As used herein, a “haloaliphatic” group refers to an aliphatic group substituted with one to three halogen. For instance, the term haloalkyl includes the group —CF₃.

As used herein, a “mercapto” or “sulfhydryl” group refers to —SH.

As used herein, a “sulfo” group refers to —SO₃H, or —SO₃R^{X} when used terminally or S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y}, and R^{Z} have been defined above.

As used herein, a “sulfonamide” group refers to the structure —S(O)₂—NR^{X}R^{Y}, or —NR^{X}—S(O)₂—R^{Z} when used terminally; or —S(O)₂—NR^{X}—, or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Examples of sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, and/or the like.

As used herein, a “sulfonyl” group refers to —S(O)₂—R^{X} when used terminally and —S(O)₂— when used internally, wherein R^{X} has been defined above. Examples of sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂—, and/or the like.

As used herein, a “sulfoxy” group refers to —O—S(O)—R^{X}, or —S(O)—O—R^{X}, when used terminally and —O—S(O)—, or —S(O)—O— when used internally, where R^{X} has been defined above.

As used herein, a “halogen” or “halo” group refers to fluorine (F), chlorine (Cl), bromine (Br), or iodine (I).

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy, aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} each when used terminally and —NR^{X}—CO—NR^{Y}— or —NR^{X}—CS—NR^{Y}— each when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} where in R^{X} and R^{Y} have been defined above.

As used herein, the term “amidino” group refers to the structure —C═(NR^{X})N(R^{X}R^{Y}) wherein R^{X} and R^{Y} have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl (i.e., R^{X}O(O)C-alkyl) is an example of a carboxy group used terminally. A group is internal when the group is present in the middle of or within the termini of a substituent of the chemical structure. Alkylcarboxy (e.g., alkyl-C(O)O— or alkyl-OC(O)—) and alkylcarboxyaryl (e.g., alkyl-C(O)O-aryl-, or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ—, where each Q is independently a hydrogen (H or —H), or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The term “protecting group” as described herein, refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

The term “optionally substituted,” unless otherwise specified, means that a group is unsubstituted or substituted by one or more (e.g., 1, 2, 3, 4, or 5) of the substituents listed for that group, in which the substituents may be the same or different. In certain embodiments, an optionally substituted group is unsubstituted. In certain embodiments, an optionally substituted group has one substituent. In certain embodiments, an optionally substituted group has two substituents. In certain embodiments, an optionally substituted group has three substituents. In certain embodiment, an optionally substituted group has four substituents. In certain embodiments, an optionally substituted group has 1 to 2, 1 to 3, 1 to 4, or 1 to 5 substituents. When multiple substituents are present, each substituent is independently chosen unless indicated otherwise. For example, each (C₁-C₄ alkyl) substituent on the group —N(C₁-C₄ alkyl)(C₁-C₄ alkyl) can be selected independently from the other, so as to generate groups such as —N(CH₃)(CH₂CH₃), etc.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, for example, both rings share one common atom. Non-limiting examples of spiro heterocycloalkyls include

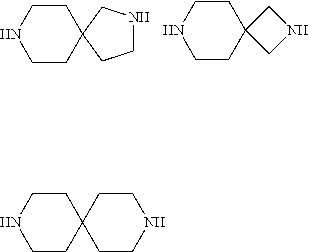 [SMILES: C1CC2(CCN1)CCNC2 ; C1CC2(CCN1)CNC2 ; C1CC2(CCN1)CCNCC2]

2,8-diazaspiro[4.5]decane; 2,7-diazaspiro[3.5]nonane; 3,9-diazaspiro[5.5]undecane;

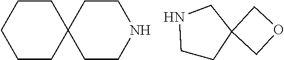

3-azaspiro[5.5]undecane; and 2-oxa-6-azaspiro[3.5]octane. Spiro compounds depicted with overlapping rings indicate that the rings can bond at any vertex. For instance, in the spiro group

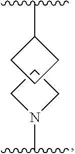

the two rings can bond at any of the three available vertex atoms in either ring.

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, and their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Unless a specific isotope of an element is indicated in a formula, the disclosure includes all isotopologues of the compounds disclosed herein, such as, for example, deuterated derivatives of the compounds (where H can be 2H, i.e., D). Isotopologues can have isotopic replacements at any or at all locations in a structure, or can have atoms present in natural abundance at any or all locations in a structure.

The disclosure also includes any or all of the stereochemical forms, including any enantiomeric or diastereomeric forms of the compounds described herein, and cis/trans or E/Z isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by the disclosure, as well as the general non-stereospecific form and mixtures of the disclosed compounds in any ratio, including mixtures of two or more stereochemical forms of a disclosed in any ratio, such that racemic, non-racemic, enantioenriched and scalemic mixtures of a compound are embraced. Compositions comprising a disclosed compound also are intended, such as a composition of substantially pure compound, including a specific stereochemical form thereof. Compositions comprising a mixture of disclosed compounds in any ratio also are embraced by the disclosure, including compositions comprising mixtures of two or more stereochemical forms of a disclosed compound in any ratio, such that racemic, non-racemic, enantioenriched and scalemic mixtures of a compound are embraced by the disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated.

The disclosure also embraces any and all tautomeric forms of the compounds described herein.

The disclosure is intended to embrace all salts of the compounds described herein, as well as methods of using such salts of the compounds. In one embodiment, the salts of the compounds comprise pharmaceutically acceptable salts. Pharmaceutically acceptable salts are those salts that can be administered as drugs or pharmaceuticals to humans and/or animals and that, upon administration, retain at least some of the biological depenactivity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, also can be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, also can be prepared. For lists of pharmaceutically acceptable salts, see, for example, P. H. Stahl and C. G. Wermuth (eds.)“Handbook of Pharmaceutical Salts, Properties, Selection and Use” Wiley-VCH, 2011 (ISBN: 978-3-90639-051-2). Several pharmaceutically acceptable salts are also disclosed in Berge, J. Pharm. Sci. 66: 1 (1977).

As used herein, the term “about” means within ±10% of a value. For example, a dose that is about 100 mg/kg provides that the dose can be 90 mg/kg to 110 mg/kg. By way of further example, an amount of an additional therapeutic agent ranging from about 50% to about 100% provides that the amount of additional therapeutic agent range from 45-55% to 90-110%. A person of skill in the art will appreciate the scope and application of the term “about” when used to describe other values disclosed herein.

Unless otherwise stated, structures depicted herein also are meant to include all isomeric (e.g., enantiomeric, diastereomeric, and geometric (or conformational)) forms of the structure; for example, the (R)- and (S)-configurations for each asymmetric center, (Z)- and (E)-double bond isomers, and (Z)- and (E)-conformational isomers. Therefore, single stereochemical isomers as well as enantiomeric, diastereomeric, and geometric (or conformational) mixtures of the present compounds are within the scope of the description. Alternatively, as used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]-[(5)-compound])/([(R)-compound]+[(S)-compound])×100, where the (R)-compound>(9-compound; or % ee=([(9-compound][(R)-compound])/([(S)-compound]+[(R)-compound])×100, where the (9-compound>(R)-compound. Moreover, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, the compounds or inhibitors described herein have an ee, de, % ee, or % de range from ninety to one hundred. In certain embodiments, the compounds or inhibitors described herein have an ee, de, % ee, or % de range from ninety-five to one hundred. In certain embodiments, the compounds or inhibitors described herein have an ee, de, % ee, or de range from ninety-seven to one hundred. In certain embodiments, the compounds or inhibitors described herein have an ee, de, % ee, or % de range from ninety-eight to one hundred. In certain embodiments, the compounds or inhibitors described herein have an ee, de, % ee, or % de range from ninety-nine to one hundred.

In one embodiment of a compound or inhibitor described herein, the ee, de, % ee, or de is one. In one embodiment of a compound or inhibitor described herein, the ee, de, % ee, or de is two. In one embodiment of a compound or inhibitor described herein, the ee, de, % ee, or de is three. In one embodiment of a compound or inhibitor described herein, the ee, de, % ee, or % de is four. In one embodiment of a compound or inhibitor described herein, the ee, de, % ee, or % de is five. In one embodiment of a compound or inhibitor described herein, the ee, de, % ee, or % de is six. In one embodiment of a compound or inhibitor described herein, the ee, de, % ee, or % de is seven. In one embodiment of a compound or inhibitor described herein, the ee, de, % ee, or % de is eight. In one embodiment of a compound or inhibitor described herein, the ee, de, % ee, or % de is nine. In one embodiment of a compound or inhibitor described herein, the ee, de, % ee, or % de is ten. In one embodiment of a compound or inhibitor described herein, the ee, de, % ee, or % de is eleven. In one embodiment of a compound or inhibitor described herein, the ee, de, % ee, or % de is twelve. In one embodiment of a compound or inhibitor described herein, the ee, de, % ee, or % de is thirteen. In one embodiment of a compound or inhibitor described herein, the ee, de, % ee, or % de is fourteen. In one embodiment of a compound or inhibitor described herein, the ee, de, % ee, or % de is fifteen. In one embodiment of a compound or inhibitor described herein, the ee, de, % ee, or % de is sixteen. In one embodiment of a compound or inhibitor described herein, the ee, de, % ee, or % de is seventeen. In one embodiment of a compound or inhibitor described herein, the ee, de, % ee, or % de is eighteen. In one embodiment of a compound or inhibitor described herein, the ee, de, % ee, or % de is nineteen. In one embodiment of a compound or inhibitor described herein, the ee, de, % ee, or % de is twenty. In one embodiment of a compound or inhibitor described herein, the ee, de, % ee, or % de is twenty-one. In one embodiment of a compound or inhibitor described herein, the ee, de, % ee, or % de is twenty-two. In one embodiment of a compound or inhibitor described herein, the ee, de, % ee, or % de is twenty-three. In one embodiment of a compound or inhibitor described herein, the ee, de, % ee, or % de is twenty-four. In one embodiment of a compound or inhibitor described herein, the ee, de, % ee, or % de is twenty-five. In one embodiment of a compound or inhibitor described herein, the ee, de, % ee, or % de is twenty-six. In one embodiment of a compound or inhibitor described herein, the ee, de, % ee, or % de is twenty-seven. In one embodiment of a compound or inhibitor described herein, the ee, de, % ee, or % de is twenty-eight. In one embodiment of a compound or inhibitor described herein, the ee, de, % ee, or % de is twenty-nine. In one embodiment of a compound or inhibitor described herein, the ee, de, % ee, or % de is thirty. In one embodiment of a compound or inhibitor described herein, the ee, de, % ee, or % de is thirty-one. In one embodiment of a compound or inhibitor described herein, the ee, de, % ee, or % de is thirty-two. In one embodiment of a compound or inhibitor described herein, the ee, de, % ee, or % de is thirty-three. In one embodiment of a compound or inhibitor described herein, the ee, de, % ee, or % de is thirty-four. In one embodiment of a compound or inhibitor described herein, the ee, de, % ee, or % de is thirty-five. In one embodiment of a compound or inhibitor described herein, the ee, de, % ee, or % de is thirty-six. In one embodiment of a compound or inhibitor described herein, the ee, de, % ee, or de is thirty-seven. In one embodiment of a compound or inhibitor described herein, the ee, de, % ee, or % de is thirty-eight. In one embodiment of a compound or inhibitor described herein, the ee, de, % ee, or % de is thirty-nine. In one embodiment of a compound or inhibitor described herein, the ee, de, % ee, or % de is forty. In one embodiment of a compound or inhibitor described herein, the ee, de, % ee, or % de is forty-one. In one embodiment of a compound or inhibitor described herein, the ee, de, % ee, or % de is forty-two. In one embodiment of a compound or inhibitor described herein, the ee, de, % ee, or % de is forty-three. In one embodiment of a compound or inhibitor described herein, the ee, de, % ee, or % de is forty-four. In one embodiment of a compound or inhibitor described herein, the ee, de, % ee, or % de is forty-five. In one embodiment of a compound or inhibitor described herein, the ee, de, % ee, or % de is forty-six. In one embodiment of a compound or inhibitor described herein, the ee, de, % ee, or % de is forty-seven. In one embodiment of a compound or inhibitor described herein, the ee, de, % ee, or % de is forty-eight. In one embodiment of a compound or inhibitor described herein, the ee, de, % ee, or % de is forty-nine. In one embodiment of a compound or inhibitor described herein, the ee, de, % ee, or % de is fifty. In one embodiment of a compound or inhibitor described herein, the ee, de, % ee, or % de is fifty-one. In one embodiment of a compound or inhibitor described herein, the ee, de, % ee, or % de is fifty-two. In one embodiment of a compound or inhibitor described herein, the ee, de, % ee, or % de is fifty-three. In one embodiment of a compound or inhibitor described herein, the ee, de, % ee, or % de is fifty-four. In one embodiment of a compound or inhibitor described herein, the ee, de, % ee, or % de is fifty-five. In one embodiment of a compound or inhibitor described herein, the ee, de, % ee, or % de is fifty-six. In one embodiment of a compound or inhibitor described herein, the ee, de, % ee, or % de is fifty-seven. In one embodiment of a compound or inhibitor described herein, the ee, de, % ee, or % de is fifty-eight. In one embodiment of a compound or inhibitor described herein, the ee, de, % ee, or % de is fifty-nine. In one embodiment of a compound or inhibitor described herein, the ee, de, % ee, or % de is sixty. In one embodiment of a compound or inhibitor described herein, the ee, de, % ee, or % de is sixty-one. In one embodiment of a compound or inhibitor described herein, the ee, de, % ee, or % de is sixty-two. In one embodiment of a compound or inhibitor described herein, the ee, de, % ee, or % de is sixty-three. In one embodiment of a compound or inhibitor described herein, the ee, de, % ee, or % de is sixty-four. In one embodiment of a compound or inhibitor described herein, the ee, de, % ee, or % de is sixty-five. In one embodiment of a compound or inhibitor described herein, the ee, de, % ee, or % de is sixty-six. In one embodiment of a compound or inhibitor described herein, the ee, de, % ee, or % de is sixty-seven. In one embodiment of a compound or inhibitor described herein, the ee, de, % ee, or % de is sixty-eight. In one embodiment of a compound or inhibitor described herein, the ee, de, % ee, or % de is sixty-nine. In one embodiment of a compound or inhibitor described herein, the ee, de, % ee, or % de is seventy. In one embodiment of a compound or inhibitor described herein, the ee, de, % ee, or % de is seventy-one. In one embodiment of a compound or inhibitor described herein, the ee, de, % ee, or % de is seventy-two. In one embodiment of a compound or inhibitor described herein, the ee, de, % ee, or % de is seventy-three. In one embodiment of a compound or inhibitor described herein, the ee, de, % ee, or % de is seventy-four. In one embodiment of a compound or inhibitor described herein, the ee, de, % ee, or % de is seventy-five. In one embodiment of a compound or inhibitor described herein, the ee, de, % ee, or % de is seventy-six. In one embodiment of a compound or inhibitor described herein, the ee, de, % ee, or % de is seventy-seven. In one embodiment of a compound or inhibitor described herein, the ee, de, % ee, or % de is seventy-eight. In one embodiment of a compound or inhibitor described herein, the ee, de, % ee, or % de is seventy-nine. In one embodiment of a compound or inhibitor described herein, the ee, de, % ee, or % de is eighty. In one embodiment of a compound or inhibitor described herein, the ee, de, % ee, or % de is eighty-one. In one embodiment of a compound or inhibitor described herein, the ee, de, % ee, or % de is eighty-two. In one embodiment of a compound or inhibitor described herein, the ee, de, % ee, or % de is eighty-three. In one embodiment of a compound or inhibitor described herein, the ee, de, % ee, or % de is eighty-four. In one embodiment of a compound or inhibitor described herein, the ee, de, % ee, or % de is eighty-five. In one embodiment of a compound or inhibitor described herein, the ee, de, % ee, or % de is eighty-six. In one embodiment of a compound or inhibitor described herein, the ee, de, % ee, or de is eighty-seven. In one embodiment of a compound or inhibitor described herein, the ee, de, % ee, or % de is eighty-eight. In one embodiment of a compound or inhibitor described herein, the ee, de, % ee, or % de is eighty-nine. In one embodiment of a compound or inhibitor described herein, the ee, de, % ee, or % de is ninety. In one embodiment of a compound or inhibitor described herein, the ee, de, % ee, or % de is ninety-one. In one embodiment of a compound or inhibitor described herein, the ee, de, % ee, or % de is ninety-two. In one embodiment of a compound or inhibitor described herein, the ee, de, % ee, or % de is ninety-three. In one embodiment of a compound or inhibitor described herein, the ee, de, % ee, or % de is ninety-four. In one embodiment of a compound or inhibitor described herein, the ee, de, % ee, or % de is ninety-five. In one embodiment of a compound or inhibitor described herein, the ee, de, % ee, or % de is ninety-six. In one embodiment of a compound or inhibitor described herein, the ee, de, % ee, or % de is ninety-seven. In one embodiment of a compound or inhibitor described herein, the ee, de, % ee, or % de is ninety-eight. In one embodiment of a compound or inhibitor described herein, the ee, de, % ee, or % de is ninety-nine. In one embodiment of a compound or inhibitor described herein, the ee, de, % ee, or % de is one hundred. In certain embodiments, compounds or inhibitors described within Table 1 herein have an ee, de, % ee, or % de as described within this paragraph. In certain embodiments, compound or inhibitor as described in the Examples and/or Biological Examples have an ee, de, % ee, or % de as described within this paragraph. Unless otherwise stated, all tautomeric forms of the compounds of the description are within the scope of the description. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this description. Such compounds are useful, for example, as analytical tools or probes in biological assays, or as therapeutic agents.

As used herein, the term “&1” means that a compound including the “&1” notation at a particular chemical element or atom (e.g., carbon) within the compound was prepared as a mixture of two stereoisomers at the noted chemical element or atom (e.g., a diastereomeric mixture having a de or % de as described above).

Chemical structures and nomenclature are derived from ChemDraw, version 19.0, Cambridge, Mass.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

As used herein, the phrase “stable or chemically feasible” refers to compounds that are not substantially altered when subjected to conditions to allow for their production, detection, and their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term an “effective amount” of an agent disclosed herein is an amount sufficient to carry out a specifically stated purpose. In certain embodiments, an “effective amount” may be determined empirically and in a routine manner, in relation to the stated purpose. In certain embodiments, an “effective amount” or an “amount sufficient” of an agent is that amount adequate to produce a desired biological effect, such as a beneficial result, including a beneficial clinical result. In some embodiments, the term “effective amount” refers to an amount of an agent effective to “treat” a disease or disorder in an individual (e.g., a mammal such as a human).

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile.

The term “excipients” as used herein include pharmaceutically acceptable excipients, carriers, vehicles or stabilizers that are nontoxic to the cell or mammal being exposed thereto at the dosages and concentrations employed. In certain embodiments, the physiologically acceptable excipient is an aqueous pH buffered solution.

The terms “treating” or “treatment” of a disease refer to executing a protocol, which may include administering one or more therapeutic agent to an individual (human or otherwise), in an effort to obtain beneficial or desired results in the individual, including clinical results. In certain embodiments beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total). In certain embodiments, “treatment” also can mean prolonging survival as compared to expected survival of an individual not receiving treatment. In certain embodiments, “treating” and “treatment” may occur by administration of one dose of a therapeutic agent or therapeutic agents, or may occur upon administration of a series of doses of a therapeutic agent or therapeutic agents. In certain embodiments, “treating” or “treatment” does not require complete alleviation of signs or symptoms, and does not require a cure. In certain embodiments, “treatment” also can refer to clinical intervention, such as administering one or more therapeutic agents to an individual, designed to alter the natural course of the individual or cell being treated (i.e., to alter the course of the individual or cell that would occur in the absence of the clinical intervention). In certain embodiments, the term “therapeutic agent” can refer to a CTM drug that induce the proteolytic degradation of a target protein, wherein said target protein is selected from the group consisting of Bruton's tyrosine kinase (BTK); tyrosine-protein kinase (ITK/TSK); bromodomain-containing protein 4 (BRD4); FMS-like tyrosine kinase 3 (FLT-3); Brg/Brahma-associated factors (BAF complex); induced myeloid leukemia cell differentiation protein (MCL-1); signal transducer and activator of transcription 3 (STAT3); barrier-to-autointegration factor (BAF); and BCR-ABL, or compositions thereof. In certain embodiments, the target protein is selected from the group consisting of SH2 domain-containing protein tyrosine phosphatase-2 (SHP-2), fibroblast growth factor receptor (FGFR), is FGFR1 fusion, FGFR3 fusion, mouse double minute 2 homolog (MDM2), is SARS-COV2 protein (e.g. Mpro, or nsp5), phosphoinositide 3-kinase (PI3K) delta, mitogen-activated protein kinase kinase (MEK), histone-lysine N-methyltransferase 2A (MLL), and mucosa-associated lymphoid tissue lymphoma translocation protein 1 (MALT1).

The term an “individual” a “patient” or a “subject” refers a mammal. In certain embodiments, a “mammal” for purposes of treatment includes humans; non-human primates; domestic and farm animals; and zoo, sports, or pet animals, such as dogs, horses, rabbits, cattle, pigs, hamsters, gerbils, mice, ferrets, rats, cats, etc. In some embodiments, the individual or subject is human.

### Heterobifunctional Compounds

Provided herein are heterobifunctional compounds. In certain embodiments, the heterobifunctional compound comprises at least two moieties. One moiety is capable of specifically binding to a target protein. In certain embodiments, the target protein is selected from the group consisting of BTK; ITK/TSK; BRD4; FLT-3; BAF; BAF complex; MCL-1; STAT3; and BCR-ABL. In certain embodiments, the target protein is selected from the group consisting of selected from the group consisting of CDK4, CDK6, SHP-2, FGFR1, FGFR3, FGFR1 fusions, FGFR3 fusions, MDM2, TRIM24, SARS-COV2, PI3K, PI3K delta, MEK, BCR-ABL, MLL, and MALT1. In certain embodiments, the target protein is selected from the group consisting of selected from the group consisting of CDK4, CDK6, SHP-2, FGFR1, FGFR3, FGFR1 fusions, FGFR3 fusions, MDM2, TRIM24, SARS-COV2, PI3K, PI3K delta, MEK, BCR-ABL, MLL, MALT1, IRAK1, and kinases (e.g., broad spectrum). The other moiety

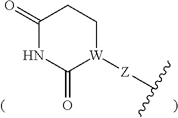

is capable of recruiting an ubiquitin ligase to degrade a target protein. In certain embodiments, the ubiquitin ligase is an E3 ligase. In certain embodiments, the ubiquitin ligase is cereblon (CRBN) or comprises cereblon as a component.

In certain embodiments, the compounds provided herein include the recited compounds as well as pharmaceutical acceptable salts, stereoisomers, tautomers, and/or mixtures thereof;

In certain embodiments, provided is a compound of Formula (I) or a stereoisomer and/or a pharmaceutically acceptable salt thereof.

In certain embodiments, Y is arylene, heteroaryl, heteroaralkyl, heteroarylene, heterocycle, heterocycloalkyl, or heterocycloalkylaryl, wherein arylene, heteroaryl, heteroaralkyl, heteroarylene, heterocycle, heterocycloalkyl, or heterocycloalkylaryl are each optionally substituted. In certain embodiments, Y moieties have the capability to bind to a target protein selected from the group consisting Bruton's tyrosine kinase (BTK); tyrosine-protein kinase (ITK/TSK); bromodomain-containing protein 4 (BRD4); FMS-like tyrosine kinase 3 (FLT-3); Brg/Brahma-associated factors (BAF complex); induced myeloid leukemia cell differentiation protein (McL-1); signal transducer and activator of transcription 3 (STAT3); barrier-to-autointegration factor (BAF); BCR-ABL; MLL; MALT1; IRAK1; and kinases to bring it into close proximity to E3 ligase to effect the protein's ubiquitination and degradation.

In one aspect, provided herein are compounds of Formula (I), and stereoisomers and pharmaceutically acceptable salt thereof:

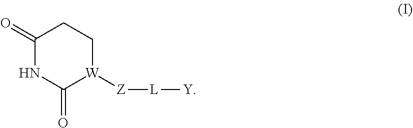

In Formula (I), W is —CH or N. In certain embodiments, W is —CH. In certain embodiments, W is —N.

In Formula (I), Z is selected from the group consisting of:

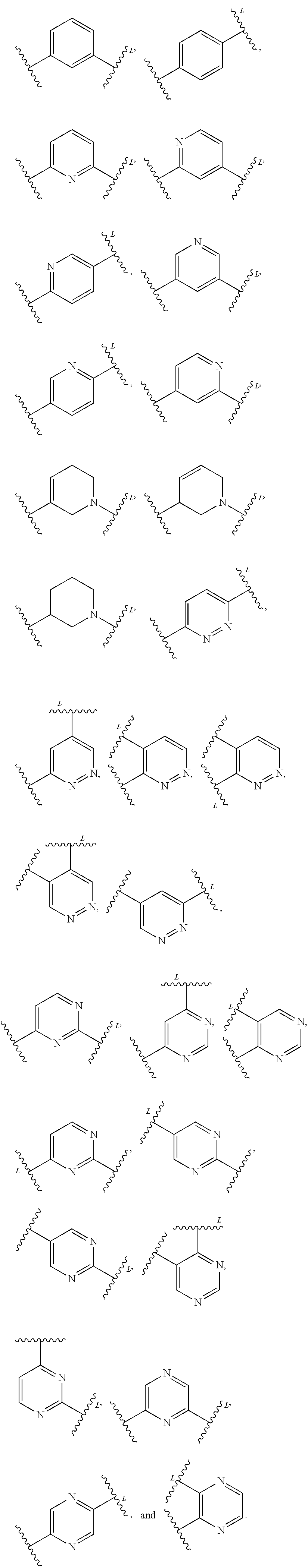

In Formula (I), Z is selected from the group consisting of:

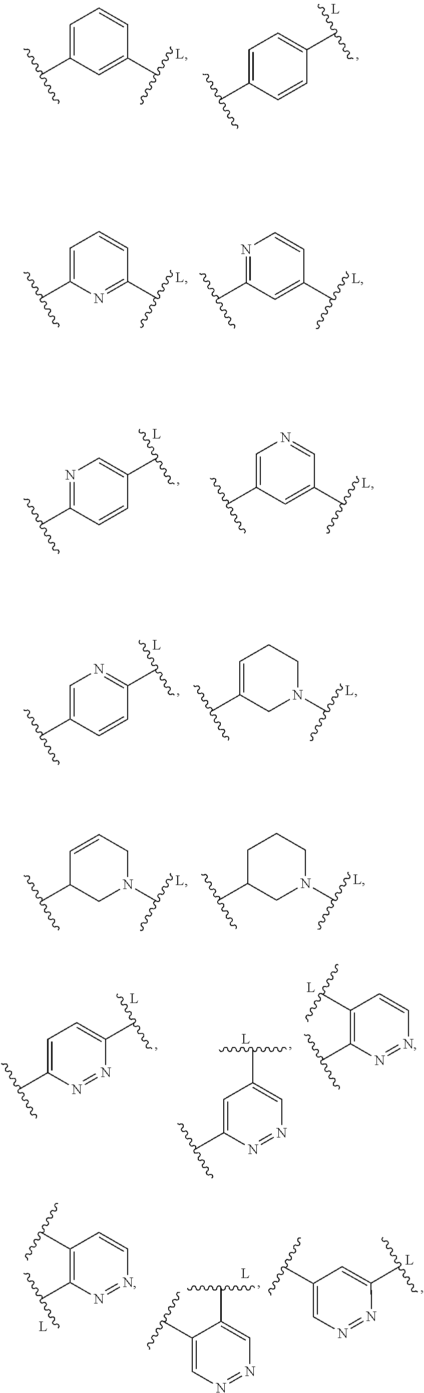

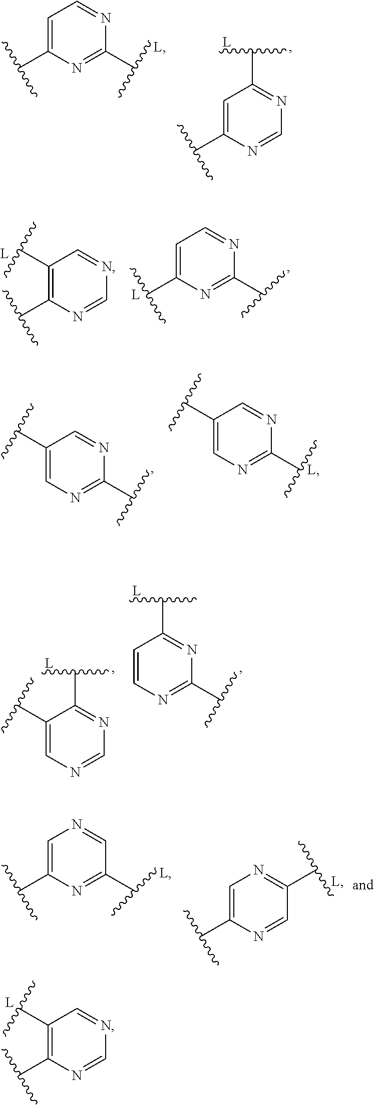

In Formula (I), L is a linker group. In certain embodiments, the linker group consists of 3 to 40 carbon atoms, including one or more acyclic and/or cyclic moieties, wherein one or more carbons atoms can be replaced optionally by a heteroatom selected from O, N, S, and P, or wherein the linker group comprises 1, 2, 3, 4, 5, 6, 7, 8, 9, or 10 ethylene glycol units.

In Formula (I), Y is a target binding moiety, which binds to a target protein which is to be degraded by the compound. In certain embodiments, Y is arylene, heteroaryl, heteroaralkyl, heteroarylene, heterocycle, heterocycloalkyl, or heterocycloalkylaryl, wherein arylene, heteroaryl, heteroaralkyl, heteroarylene, heterocycle, heterocycloalkyl, or heterocycloalkylaryl, are each optionally substituted.

In certain embodiments, provided herein are compounds of Formula (II), or a stereoisomer and/or a pharmaceutically acceptable salt thereof:

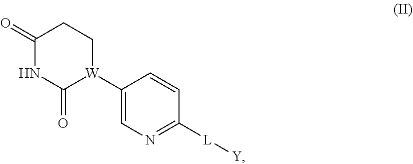

wherein the variables are as described below.

In certain embodiments, provided herein are compounds of Formula (III), or a stereoisomer and/or a pharmaceutically acceptable salt thereof:

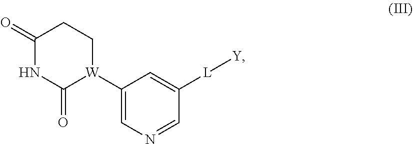

wherein the variables are as described below.

In certain embodiments, provided herein are compounds of Formula (IV), or a stereoisomer and/or a pharmaceutically acceptable salt thereof:

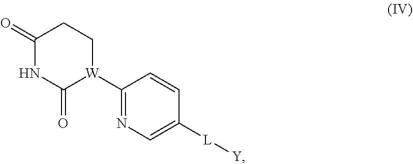

wherein the variables are as described below.

In certain embodiments, provided herein are compounds of Formula (V), or a stereoisomer and/or a pharmaceutically acceptable salt thereof:

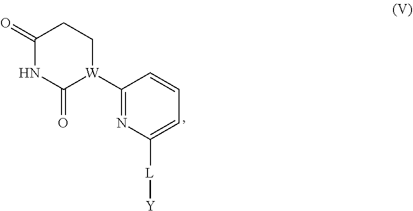

wherein the variables are as described below.

In certain embodiments, provided herein are compounds of Formula (VI), or a stereoisomer and/or a pharmaceutically acceptable salt thereof:

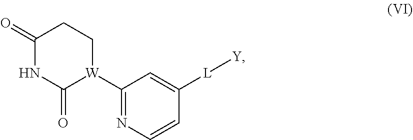

wherein the variables are as described below.

In certain embodiments, provided herein are compounds of Formula (VII), or a stereoisomer and/or a pharmaceutically acceptable salt thereof:

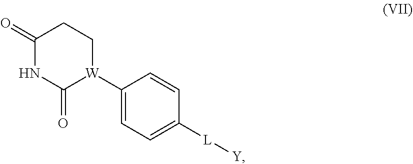

wherein the variables are as described below.

In certain embodiments, provided herein are compounds of Formula (VIII), or a stereoisomer and/or a pharmaceutically acceptable salt thereof:

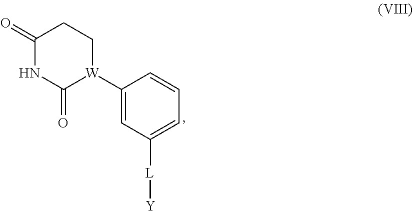

wherein the variables are as described below.

In certain embodiments, provided herein are compounds of Formula (IX), or a stereoisomer and/or a pharmaceutically acceptable salt thereof:

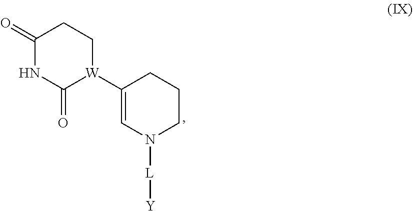

wherein the variables are as described below.

In certain embodiments, provided herein are compounds of Formula (X), or a stereoisomer and/or a pharmaceutically acceptable salt thereof.

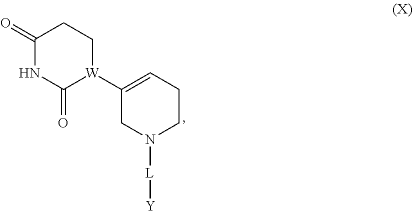

wherein the variables are as described below.

In certain embodiments, provided herein are compounds of Formula (XI), or a stereoisomer and/or a pharmaceutically acceptable salt thereof:

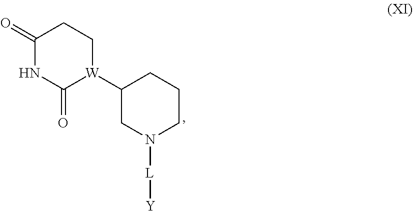

wherein the variables are as described below.

In certain embodiments, provided herein are compounds of Formula (XII), or a stereoisomer and/or a pharmaceutically acceptable salt thereof:

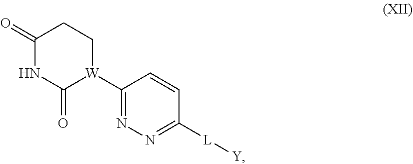

wherein the variables are as described below.

In certain embodiments, provided herein are compounds of Formula (XIII), or a stereoisomer and/or a pharmaceutically acceptable salt thereof:

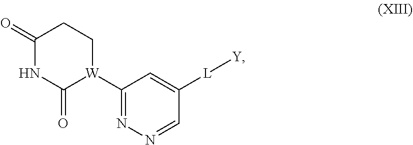

wherein the variables are as described below.

In certain embodiments, provided herein are compounds of Formula (XIV), or a stereoisomer and/or a pharmaceutically acceptable salt thereof:

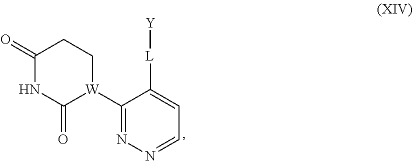

wherein the variables are as described below.

In certain embodiments, provided herein are compounds of Formula (XV), or a stereoisomer and/or a pharmaceutically acceptable salt thereof:

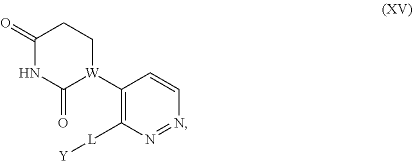

wherein the variables are as described below.

In certain embodiments, provided herein are compounds of Formula (XVI), or a stereoisomer and/or a pharmaceutically acceptable salt thereof:

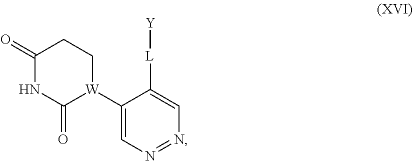

wherein the variables are as described below.

In certain embodiments, provided herein are compounds of Formula (XVII), or a stereoisomer and/or a pharmaceutically acceptable salt thereof:

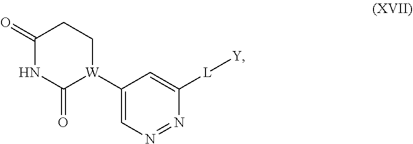

wherein the variables are as described below.

In certain embodiments, provided herein are compounds of Formula (XVIII), or a stereoisomer and/or a pharmaceutically acceptable salt thereof:

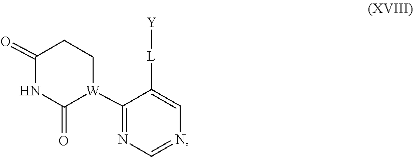

wherein the variables are as described below.

In certain embodiments, provided herein are compounds of Formula (XIX), or a stereoisomer and/or a pharmaceutically acceptable salt thereof:

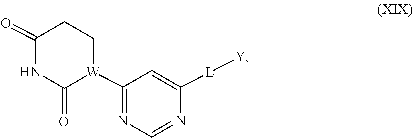

wherein the variables are as described below.

In certain embodiments, provided herein are compounds of Formula (XX), or a stereoisomer and/or a pharmaceutically acceptable salt thereof:

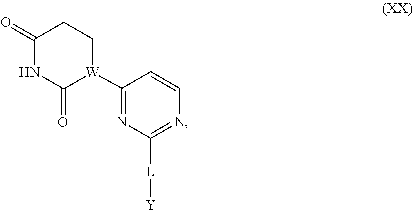

wherein the variables are as described below.

In certain embodiments, provided herein are compounds of Formula (XXI), or a stereoisomer and/or a pharmaceutically acceptable salt thereof:

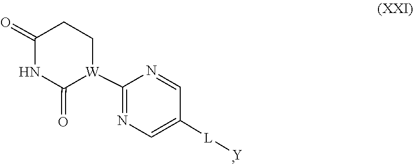

wherein the variables are as described below.

In certain embodiments, provided herein are compounds of Formula (XXII), or a stereoisomer and/or a pharmaceutically acceptable salt thereof:

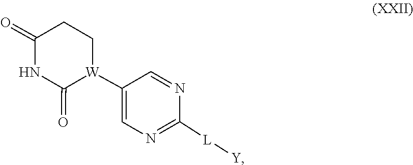

wherein the variables are as described below.

In certain embodiments, provided herein are compounds of Formula (XXIII), or a stereoisomer and/or a pharmaceutically acceptable salt thereof:

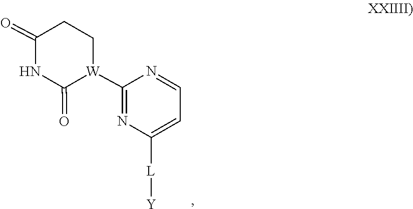

wherein the variables are as described below.

In certain embodiments, provided herein are compounds of Formula (XXIV), or a stereoisomer and/or a pharmaceutically acceptable salt thereof:

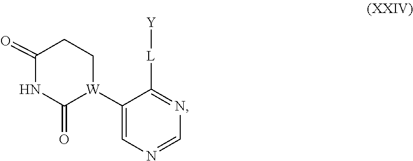

wherein the variables are as described below.

In certain embodiments, provided herein are compounds of Formula (XXV), or a stereoisomer and/or a pharmaceutically acceptable salt thereof:

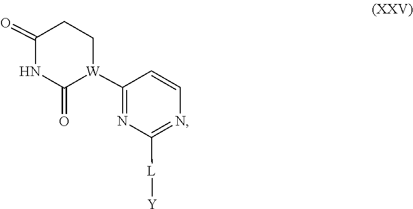

wherein the variables are as described below.

In certain embodiments, provided herein are compounds of Formula (XXVI), or a stereoisomer and/or a pharmaceutically acceptable salt thereof:

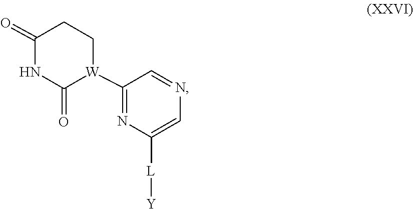

wherein the variables are as described below.

In certain embodiments, provided herein are compounds of Formula (XXVII), or a stereoisomer and/or a pharmaceutically acceptable salt thereof:

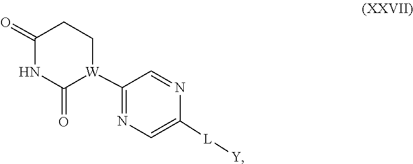

wherein the variables are as described below.

In certain embodiments, provided herein are compounds of Formula (XXVIII), or a stereoisomer and/or a pharmaceutically acceptable salt thereof:

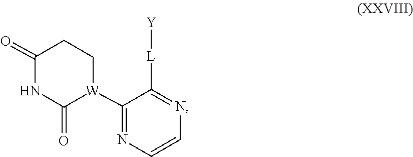

wherein the variables are as described below.

In certain embodiments, provided herein are compounds of Formula (XXIX), or a stereoisomer and/or a pharmaceutically acceptable salt thereof:

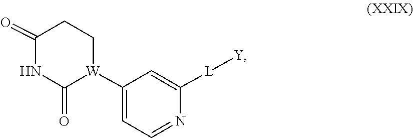

wherein the variables are as described below.

In certain embodiments, of any compound of Formula (I to XXIX), Y is selected from:

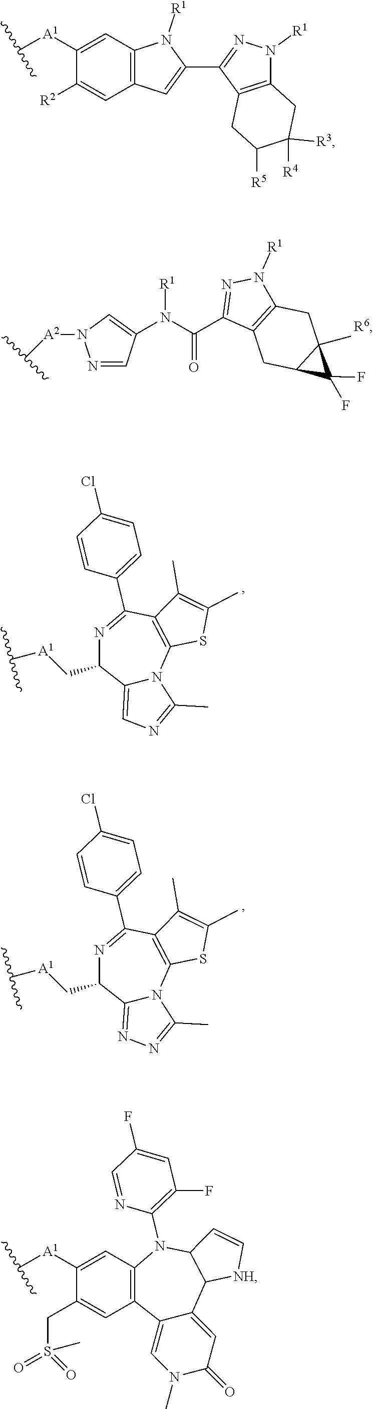

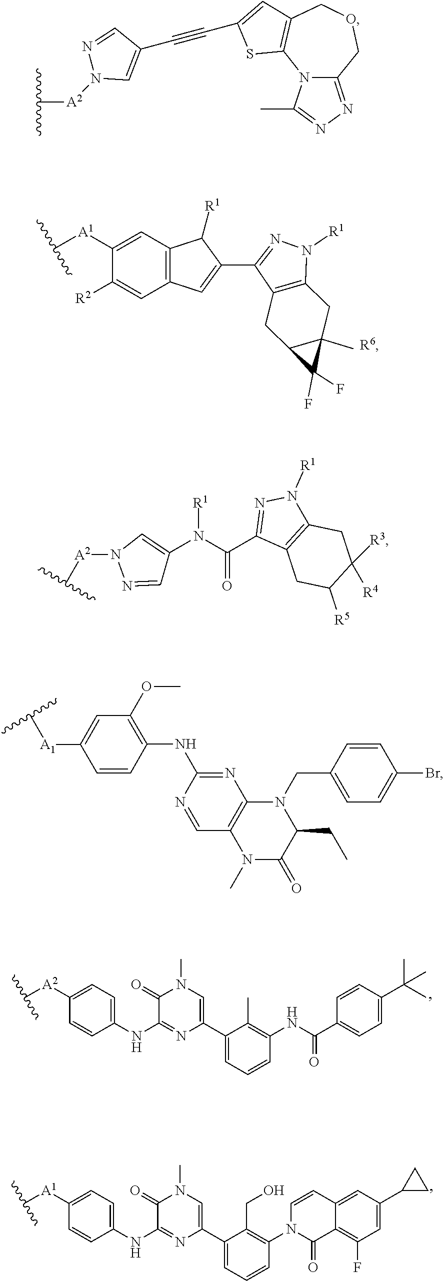

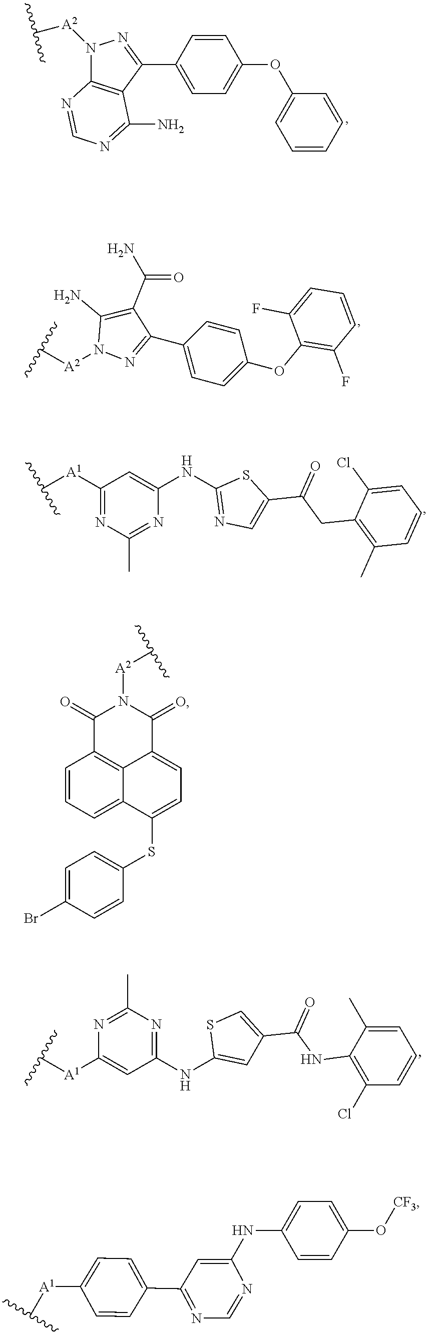

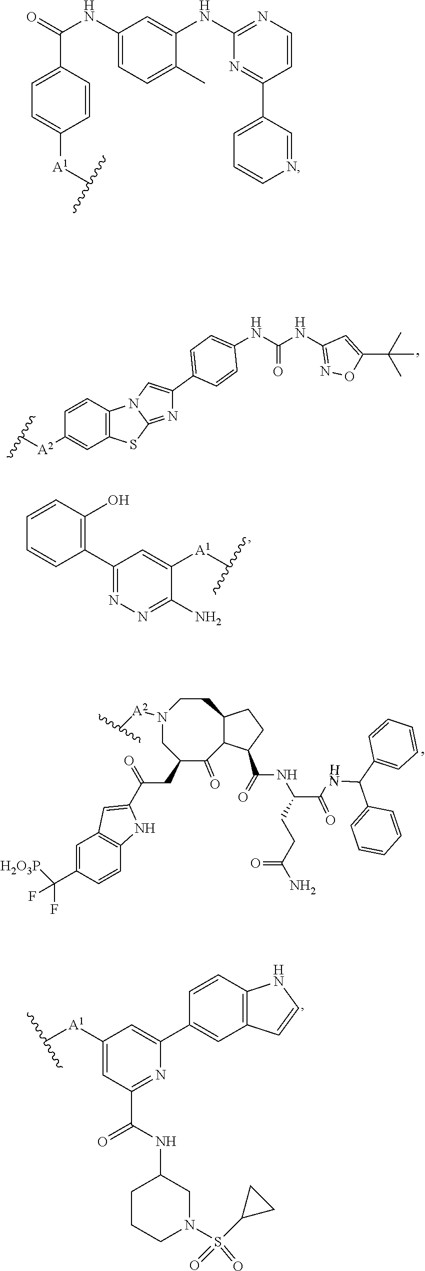

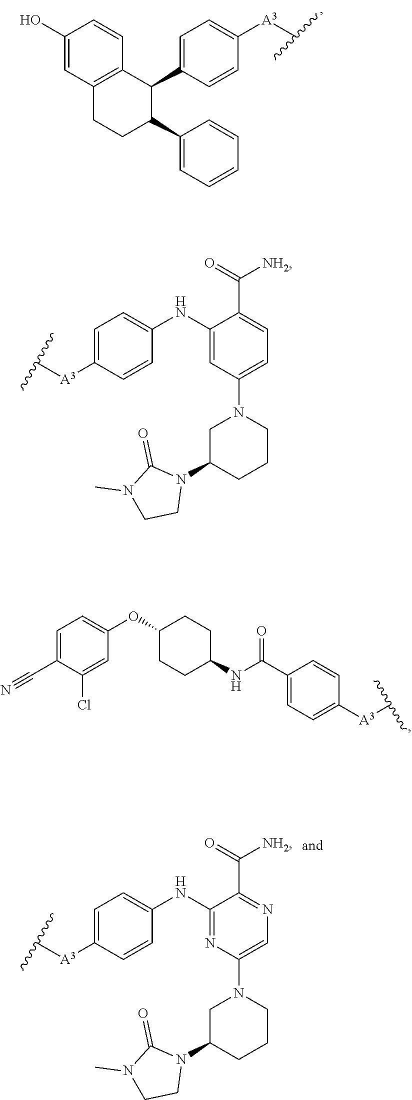

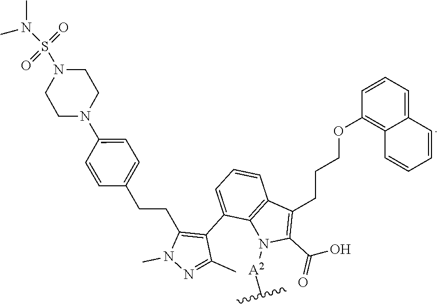

In certain embodiments, R¹ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, or —C(O)heteroaryl. In certain embodiments, R² is hydrogen, or halogen. In certain embodiments, A¹ is absent, or —O—, —C(O)—, —C(O)—NH—, —C(O)—N(CH₃)—, —C(O)—NH—R^{4a}—, —CH(A^{2a})-, —N(alkyl)-, -alkyl-. In certain embodiments, A² is absent or —C(H)(A^{2a})-. In certain embodiments, A^{2a} is hydrogen, alkyl, aryl, heterocycle, or heteroaryl. In certain embodiments, A³ is a bond. In certain embodiments, R³ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, or —C(O)heteroaryl. In certain embodiments, R⁴ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, —C(O)heteroaryl, or an alkylene bound to R⁵ to form a substituted cycloalkyl. In certain embodiments, R⁵ is hydrogen, or an alkylene bound to R⁴ to form substituted cycloalkyl. In certain embodiments, R⁵ and R⁴ form the substituted cycloalkyl. In certain embodiments, the cycloalkyl is substituted with one or more substituents selected from the group consisting of: alkyl, alkenyl, alkynyl, cycloalkyl, heterocycloalkyl, aryl, alkaryl, aralkyl, haloalkyl, heteroaryl, cyano, —C(O)alkyl, —C(O)aryl, —C(O)heteroaryl, and halogen. In certain embodiments, R⁶ is hydrogen, or alkyl. In certain embodiments, R¹ is —H. In certain embodiments, R² is —H. In certain embodiments, A¹ is —C(O)—. In certain embodiments, A¹ is —C(O)—NH—. In certain embodiments, A¹ is —CH₂—. In certain embodiments, A¹ is —N(CH₃)—. In certain embodiments, A¹ is —O—. In certain embodiments, A¹ is —C(O)—N(CH₃)—. In certain embodiments, A¹ is —C(O)—NH-phenol-. In certain embodiments, A² is —CH₂—. In certain embodiments, A² is —CH₂CH₃—. In certain embodiments, A² is —CH(CH₃)—. In certain embodiments, A² is —CH(phenyl)-.

In certain embodiments, provided herein are compounds of Formula (I), and (II-XXIX), wherein Y is:

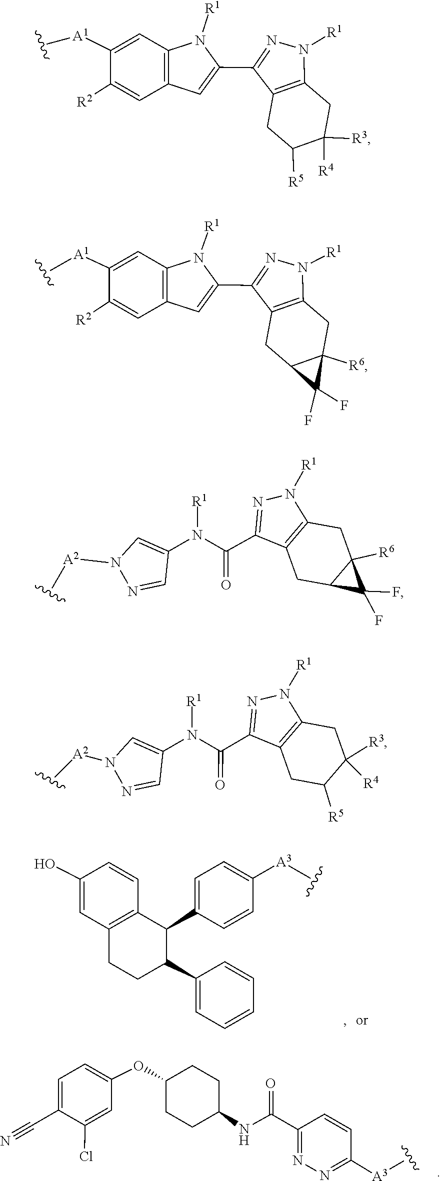

In certain embodiments, each of IV R² and IV is H. In certain embodiments, each of R³, R⁴, and R⁶ is CH₃.

In certain embodiments, provided herein are compounds of Formula (I), wherein Y is:

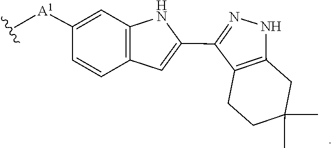

In certain embodiments, provided herein are compounds of Formula (I), wherein Y is:

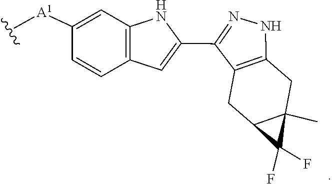

In certain embodiments, provided herein are compounds of Formula (I), wherein Y is:

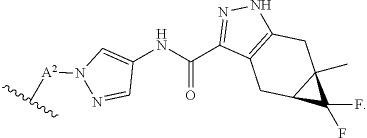

In certain embodiments, provided herein are compounds of Formula (I), wherein Y is:

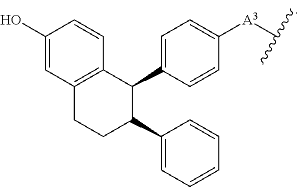

In certain embodiments, provided herein are compounds of Formula (I), wherein Y is:

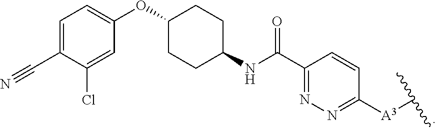

In certain embodiments, provided herein are compounds of Formula (I), wherein Y is:

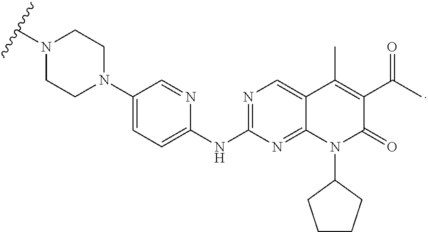 [SMILES: *N1CCN(c2ccc(Nc3ncc4c(C)c(C(C)=O)c(=O)n(C5CCCC5)c4n3)nc2)CC1]

In certain embodiments, provided herein are compounds of Formula (I), wherein Y is:

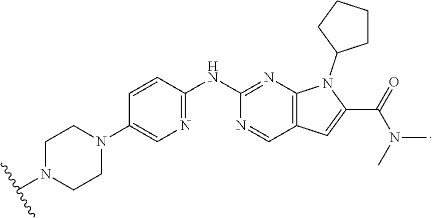 [SMILES: *N1CCN(c2ccc(Nc3ncc4cc(C(=O)N(C)C)n(C5CCCC5)c4n3)nc2)CC1]

In certain embodiments, provided herein are compounds of Formula (I), wherein Y is:

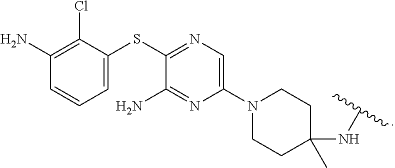 [SMILES: *NC1(C)CCN(c2cnc(Sc3cccc(N)c3Cl)c(N)n2)CC1]

In certain embodiments, provided herein are compounds of Formula (I), wherein Y is:

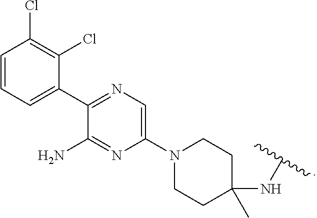 [SMILES: *NC1(C)CCN(c2cnc(-c3cccc(Cl)c3Cl)c(N)n2)CC1]

In certain embodiments, provided herein are compounds of Formula (I), wherein Y is:

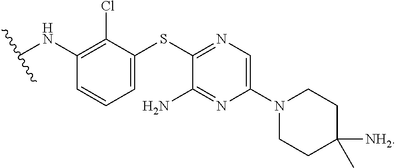 [SMILES: *Nc1cccc(Sc2ncc(N3CCC(C)(N)CC3)nc2N)c1Cl]

In certain embodiments, provided herein are compounds of Formula (I), wherein Y is:

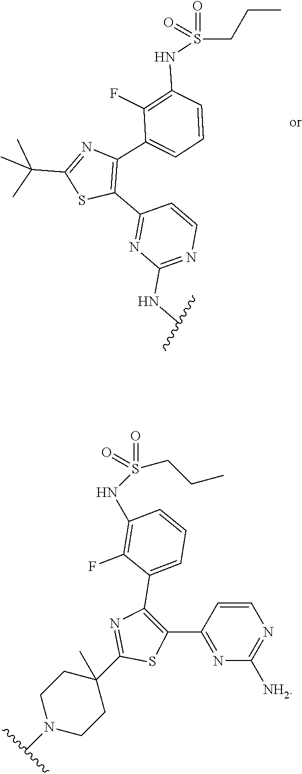 [SMILES: *Nc1nccc(-c2sc(C(C)(C)C)nc2-c2cccc(NS(=O)(=O)CCC)c2F)n1 ; *N1CCC(C)(c2nc(-c3cccc(NS(=O)(=O)CCC)c3F)c(-c3ccnc(N)n3)s2)CC1]

In certain embodiments, the target is B-Raf.

In certain embodiments, provided herein are compounds of Formula (I), wherein Y is:

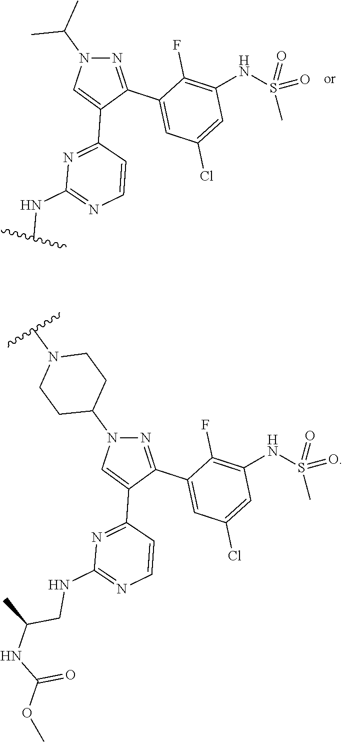 [SMILES: *Nc1nccc(-c2cn(C(C)C)nc2-c2cc(Cl)cc(NS(C)(=O)=O)c2F)n1 ; *N1CCC(n2cc(-c3ccnc(NC[C@H](C)NC(=O)OC)n3)c(-c3cc(Cl)cc(NS(C)(=O)=O)c3F)n2)CC1]

In certain embodiments, the target is B-Raf.

In certain embodiments, provided herein are compounds of Formula (I), wherein Y is:

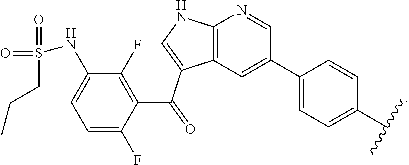 [SMILES: *c1ccc(-c2cnc3[nH]cc(C(=O)c4c(F)ccc(NS(=O)(=O)CCC)c4F)c3c2)cc1]

In certain embodiments, the target is B-Raf.

In certain embodiments, provided herein are compounds of Formula (I), wherein Y is:

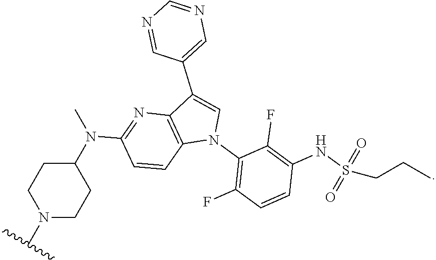 [SMILES: *N1CCC(N(C)c2ccc3c(n2)c(-c2cncnc2)cn3-c2c(F)ccc(NS(=O)(=O)CCC)c2F)CC1]

In certain embodiments, the target is B-Raf.

In certain embodiments, provided herein are compounds of Formula (I), wherein Y is:

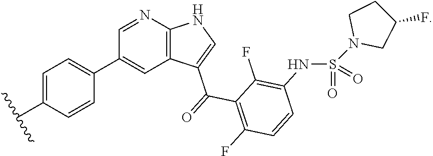 [SMILES: *c1ccc(-c2cnc3[nH]cc(C(=O)c4c(F)ccc(NS(=O)(=O)N5CC[C@H](F)C5)c4F)c3c2)cc1]

In certain embodiments, the target is B-Raf.

In certain embodiments, provided herein are compounds of Formula (I), wherein Y is:

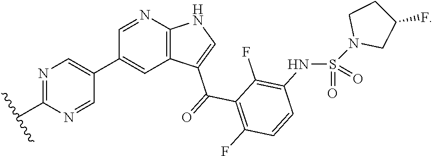 [SMILES: *c1ncc(-c2cnc3[nH]cc(C(=O)c4c(F)ccc(NS(=O)(=O)N5CC[C@H](F)C5)c4F)c3c2)cn1]

In certain embodiments, the target is B-Raf.

In certain embodiments, provided herein are compounds of Formula (I), wherein Y is:

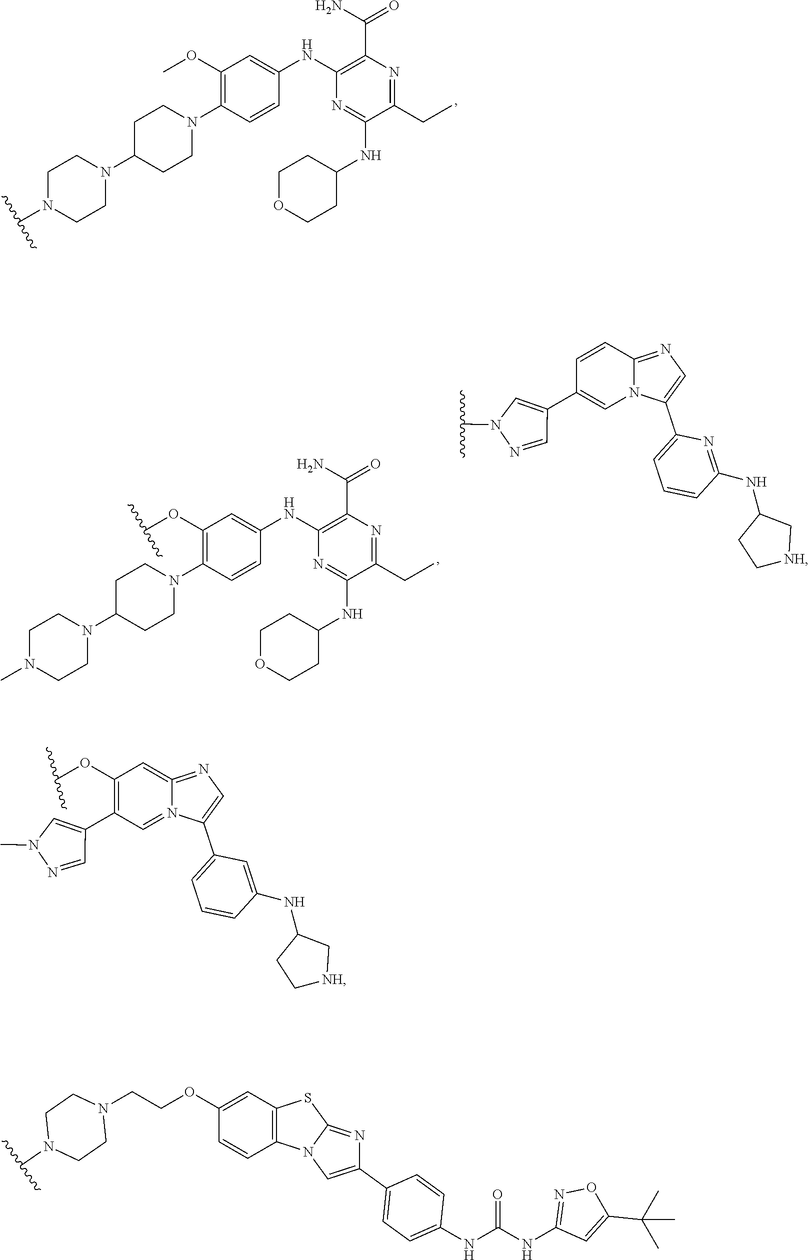

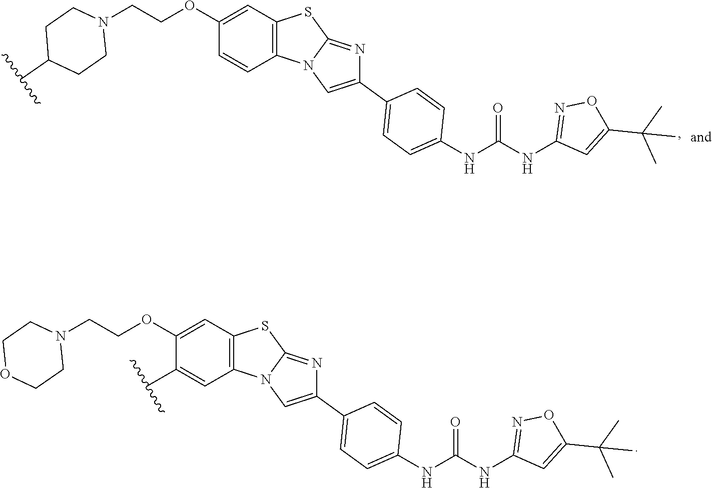 [SMILES: *C1CCN(CCOc2ccc3c(c2)sc2nc(-c4ccc(NC(=O)Nc5cc(C(C)(C)C)on5)cc4)cn23)CC1 ; *c1cc2c(cc1OCCN1CCOCC1)sc1nc(-c3ccc(NC(=O)Nc4cc(C(C)(C)C)on4)cc3)cn12]

In certain embodiments, the target is FLT3.

In certain embodiments, provided herein are compounds of Formula (I), wherein Y is:

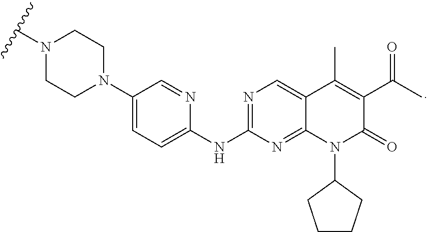 [SMILES: *N1CCN(c2ccc(Nc3ncc4c(C)c(C(C)=O)c(=O)n(C5CCCC5)c4n3)nc2)CC1]

In certain embodiments, the target is CDK4. In certain embodiments, the target is CDK6.

In certain embodiments, provided herein are compounds of Formula (I), wherein Y is:

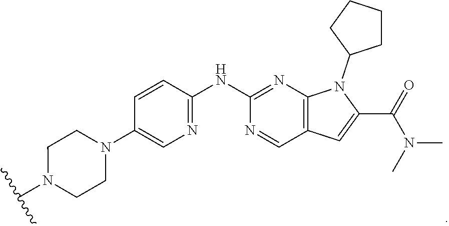 [SMILES: *N1CCN(c2ccc(Nc3ncc4cc(C(=O)N(C)C)n(C5CCCC5)c4n3)nc2)CC1]

In certain embodiments, the target is CDK4. In certain embodiments, the target is CDK6.

In certain embodiments, provided herein are compounds of Formula (I), wherein Y is:

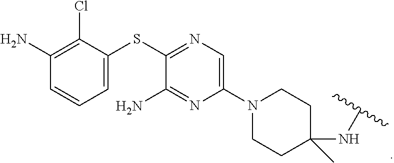 [SMILES: *NC1(C)CCN(c2cnc(Sc3cccc(N)c3Cl)c(N)n2)CC1]

In certain embodiments, the target is SHP-2.

In certain embodiments, provided herein are compounds of Formula (I), wherein Y is:

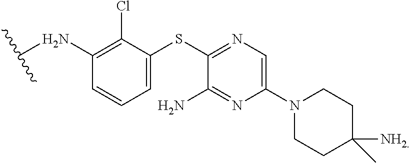 [SMILES: *Nc1cccc(Sc2ncc(N3CCC(C)(N)CC3)nc2N)c1Cl]

In certain embodiments, the target is SHP-2.

In certain embodiments, provided herein are compounds of Formula (I), wherein Y is:

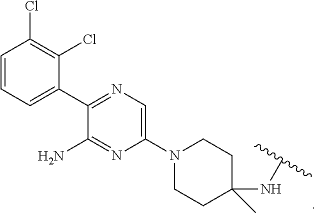 [SMILES: *NC1(C)CCN(c2cnc(-c3cccc(Cl)c3Cl)c(N)n2)CC1]

In certain embodiments, the target is SHP-2.

In certain embodiments, provided herein are compounds of Formula (I), wherein Y is:

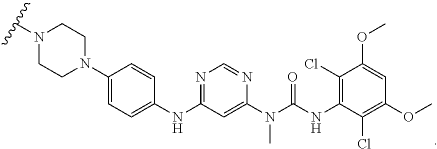 [SMILES: *N1CCN(c2ccc(Nc3cc(N(C)C(=O)Nc4c(Cl)c(OC)cc(OC)c4Cl)ncn3)cc2)CC1]

In certain embodiments, the target is FGFR1 fusion. In certain embodiments, the target is FGFR3 fusion.

In certain embodiments, provided herein are compounds of Formula (I), wherein Y is:

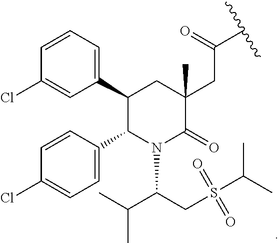 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In certain embodiments, the target is MDM2.

In certain embodiments, provided herein are compounds of Formula (I), wherein Y is:

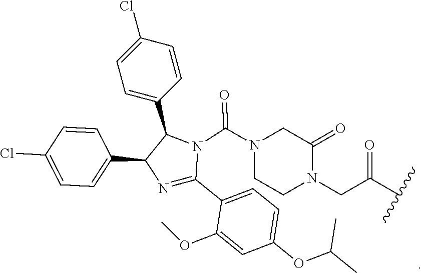 [SMILES: *C(=O)CN1CCN(C(=O)N2C(c3ccc(OC(C)C)cc3OC)=N[C@@H](c3ccc(Cl)cc3)[C@H]2c2ccc(Cl)cc2)CC1=O]

In certain embodiments, the target is MDM2.

In certain embodiments, provided herein are compounds of Formula (I), wherein Y is:

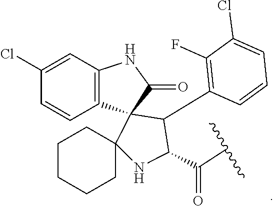 [SMILES: *C(=O)[C@@H]1NC2(CCCCC2)[C@@]2(C(=O)Nc3cc(Cl)ccc32)C1c1cccc(Cl)c1F]

In certain embodiments, the target is MDM2.

In certain embodiments, provided herein are compounds of Formula (I), wherein Y is:

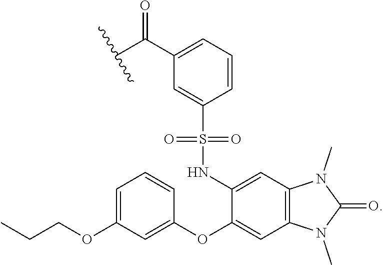

In certain embodiments, the target is TRIM24.

In certain embodiments, provided herein are compounds of Formula (I), wherein Y is:

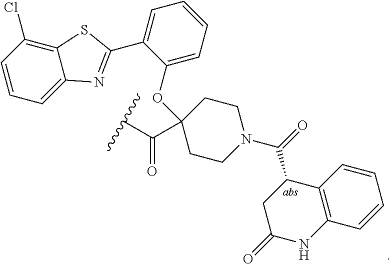 [SMILES: *C(=O)C1(Oc2ccccc2-c2nc3cccc(Cl)c3s2)CCN(C(=O)[C@H]2CC(=O)Nc3ccccc32)CC1]

In certain embodiments, the target is SARS-COV2. In certain embodiments, the protein is SARS-COV2 Mpro, or nsp5.

In certain embodiments, provided herein are compounds of Formula (I), wherein Y is:

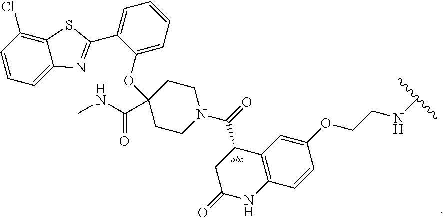 [SMILES: *NCCOc1ccc2c(c1)[C@@H](C(=O)N1CCC(Oc3ccccc3-c3nc4cccc(Cl)c4s3)(C(=O)NC)CC1)CC(=O)N2]

In certain embodiments, the target is SARS-COV2. In certain embodiments, the target is SARS-COV2. In certain embodiments, the protein is SARS-COV2 Mpro, or nsp5.

In certain embodiments, provided herein are compounds of Formula (I), wherein Y is:

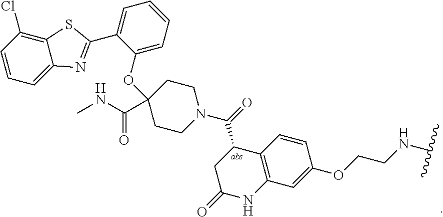 [SMILES: *NCCOc1ccc2c(c1)NC(=O)C[C@@H]2C(=O)N1CCC(Oc2ccccc2-c2nc3cccc(Cl)c3s2)(C(=O)NC)CC1]

In certain embodiments, the target is SARS-COV2. In certain embodiments, the target is SARS-COV2. In certain embodiments, the protein is SARS-COV2 Mpro, or nsp5.

In certain embodiments, provided herein are compounds of Formula (I), wherein Y is:

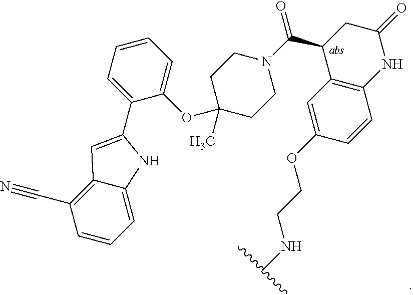 [SMILES: *NCCOc1ccc2c(c1)[C@@H](C(=O)N1CCC(C)(Oc3ccccc3-c3cc4c(C#N)cccc4[nH]3)CC1)CC(=O)N2]

In certain embodiments, the target is SARS-COV2. In certain embodiments, the target is SARS-COV2. In certain embodiments, the protein is SARS-COV2 Mpro, or nsp5.

In certain embodiments, provided herein are compounds of Formula (I), wherein Y is:

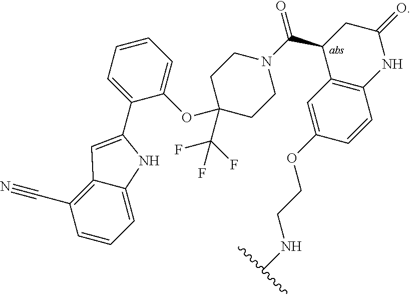 [SMILES: *NCCOc1ccc2c(c1)[C@@H](C(=O)N1CCC(Oc3ccccc3-c3cc4c(C#N)cccc4[nH]3)(C(F)(F)F)CC1)CC(=O)N2]

In certain embodiments, the target is SARS-COV2. In certain embodiments, the target is SARS-COV2. In certain embodiments, the protein is SARS-COV2 Mpro, or nsp5.

In certain embodiments, provided herein are compounds of Formula (I), wherein Y is:

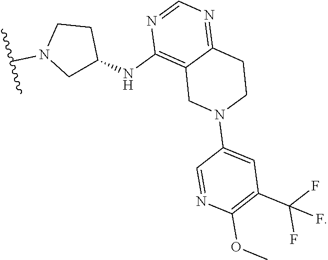 [SMILES: *N1CC[C@H](Nc2ncnc3c2CN(c2cnc(OC)c(C(F)(F)F)c2)CC3)C1]

In certain embodiments, the target is PI3K delta.

In certain embodiments, provided herein are compounds of Formula (I), wherein Y is:

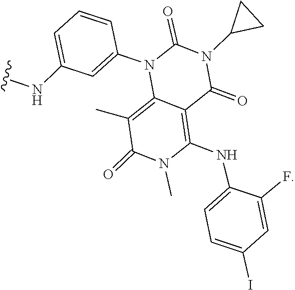

In certain embodiments, the target is MEK.

In certain embodiments, provided herein are compounds of Formula (I), wherein Y is:

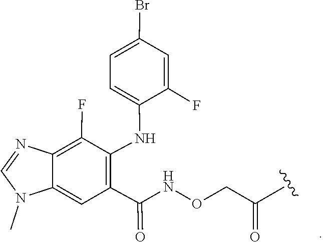

In certain embodiments, the target is MEK.

In certain embodiments, provided herein are compounds of Formula (I), wherein Y is:

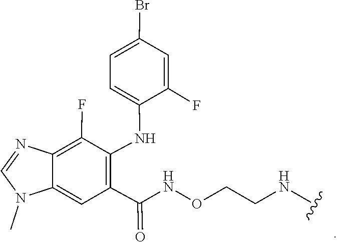

In certain embodiments, the target is MEK.

In certain embodiments, provided herein are compounds of Formula (I), wherein Y is:

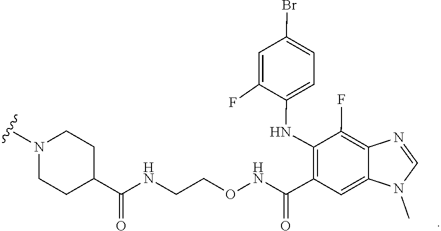

In certain embodiments, the target is MEK.

In certain embodiments, provided herein are compounds of Formula (I), wherein Y is:

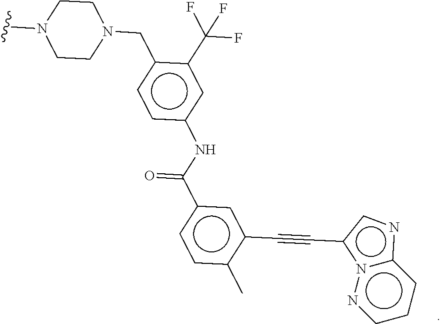

In certain embodiments, the target is BCR-ABL.

In certain embodiments, provided herein are compounds of Formula (I), wherein Y is:

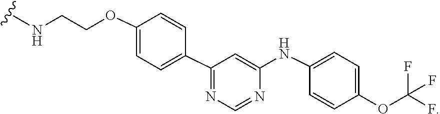

In certain embodiments, the target is BCR-ABL.

In certain embodiments, provided herein are compounds of Formula (I), wherein Y is:

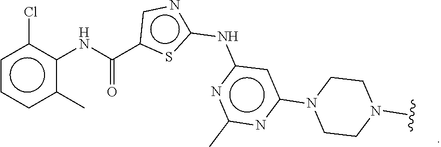

In certain embodiments, the target is BCR-ABL.

In certain embodiments, provided herein are compounds of Formula (I), wherein Y is:

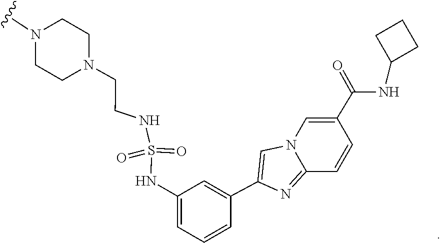

In certain embodiments, the target is MLL.

In certain embodiments, provided herein are compounds of Formula (I), wherein Y is:

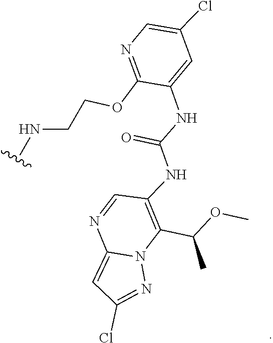

In certain embodiments, the target is MALT1.

In certain embodiments, provided herein are compounds of Formula (I), wherein Y is:

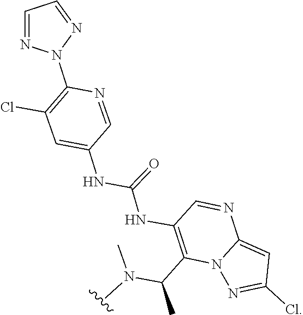

In certain embodiments, the target is MALT1.

In certain embodiments, provided herein are compounds of Formula (I), wherein Y is:

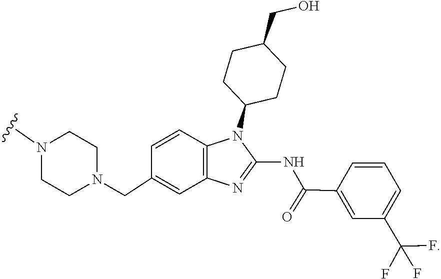

In certain embodiments, the target is IRAK1.

In certain embodiments, provided herein are compounds of Formula (I), wherein Y is:

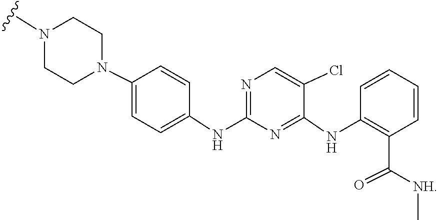

In certain embodiments, the target is a kinase. In certain embodiments, the target is one or more kinases. In certain embodiments, the target is a plurality of kinases.

In certain embodiments, provided herein are compounds of Formula (I), wherein Y is:

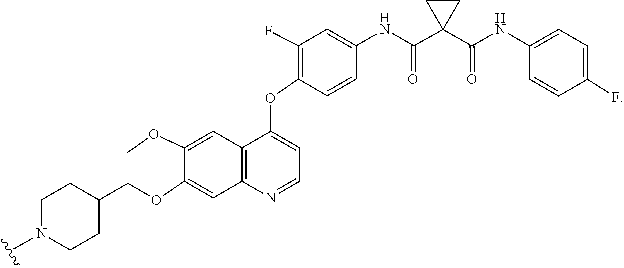

In certain embodiments, the target is a kinase. In certain embodiments, the target is one or more kinases. In certain embodiments, the target is a plurality of kinases.

In certain embodiments, provided herein are compounds of Formula (Ia), or a stereoisomer and/or a pharmaceutically acceptable salt thereof:

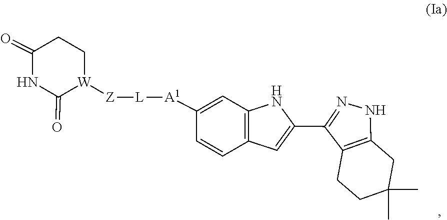

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (Ib), or a stereoisomer and/or a pharmaceutically acceptable salt thereof:

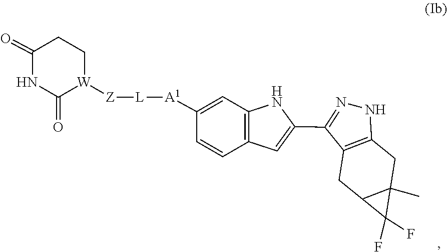

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (Ib1), or a stereoisomer and/or a pharmaceutically acceptable salt thereof:

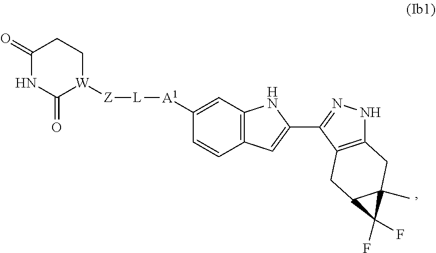

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (Ic), or a stereoisomer and/or a pharmaceutically acceptable salt thereof:

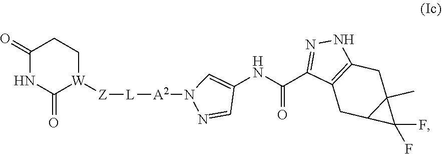

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (Id), or a stereoisomer and/or a pharmaceutically acceptable salt thereof:

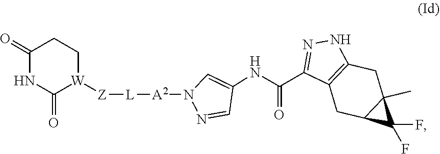

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (Ie), or a stereoisomer and/or a pharmaceutically acceptable salt thereof:

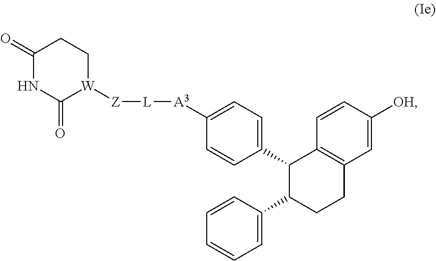

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (If), or a stereoisomer and/or a pharmaceutically acceptable salt thereof:

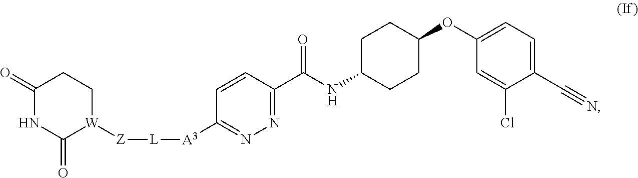

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIa), or a stereoisomer and/or a pharmaceutically acceptable salt thereof:

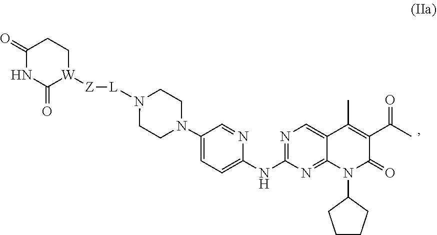

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIb), or a stereoisomer and/or a pharmaceutically acceptable salt thereof:

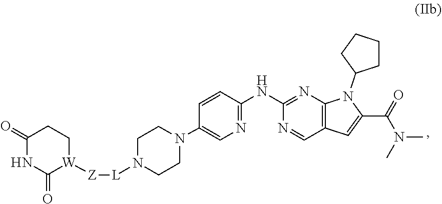

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIc), or a stereoisomer and/or a pharmaceutically acceptable salt thereof:

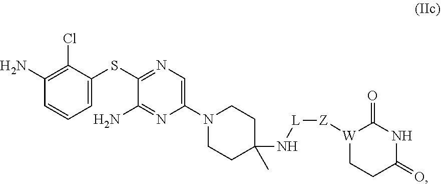

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IId), or a stereoisomer and/or a pharmaceutically acceptable salt thereof:

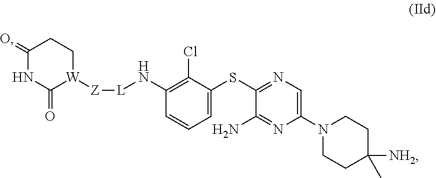

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIe), or a stereoisomer and/or a pharmaceutically acceptable salt thereof:

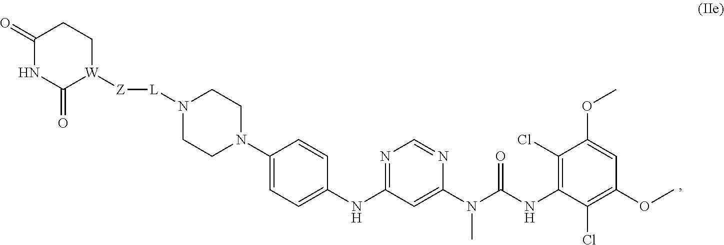

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIf), or a stereoisomer and/or a pharmaceutically acceptable salt thereof:

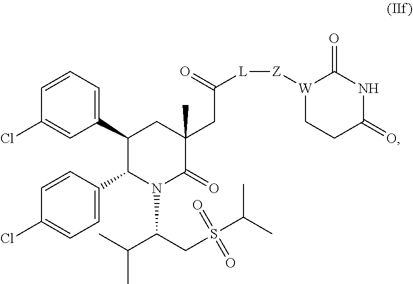

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIg), or a stereoisomer and/or a pharmaceutically acceptable salt thereof:

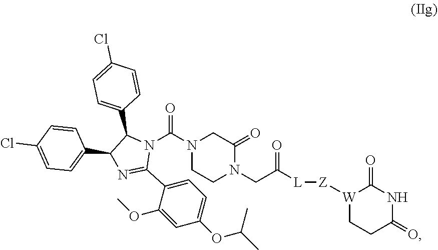

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIh), or a stereoisomer and/or a pharmaceutically acceptable salt thereof:

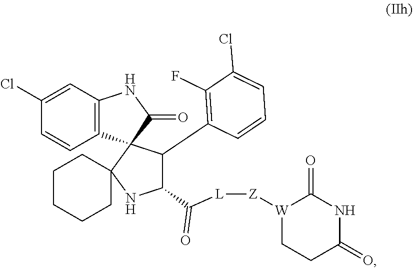

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIf), or a stereoisomer and/or a pharmaceutically acceptable salt thereof:

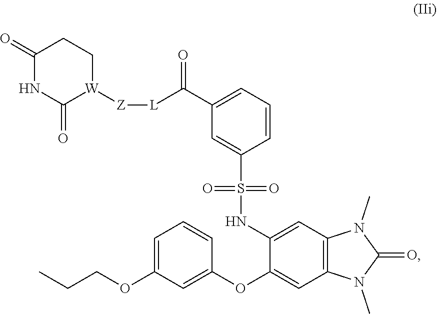

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIj), or a stereoisomer and/or a pharmaceutically acceptable salt thereof:

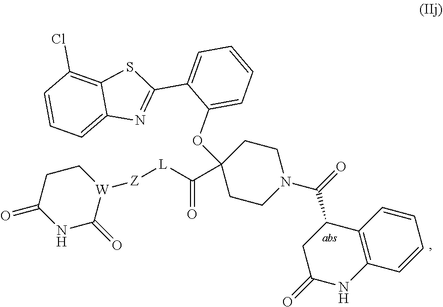

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIk), or a stereoisomer and/or a pharmaceutically acceptable salt thereof:

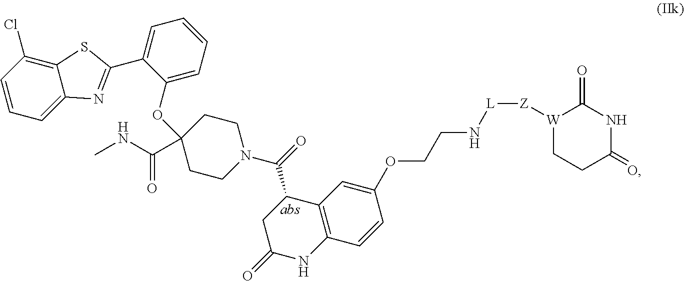

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIm), or a stereoisomer and/or a pharmaceutically acceptable salt thereof:

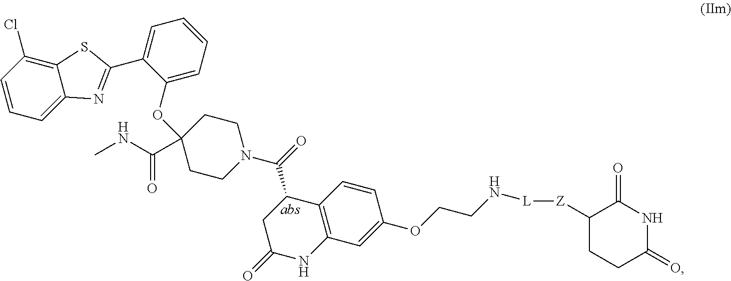

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIn), or a stereoisomer and/or a pharmaceutically acceptable salt thereof:

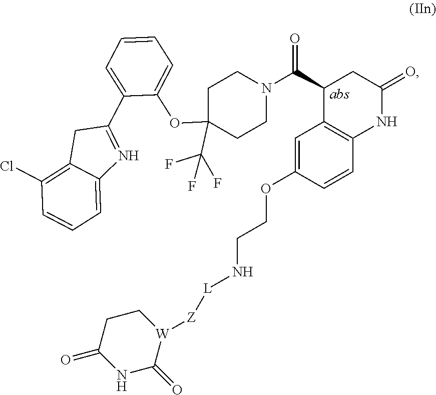

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIn′), or a stereoisomer and/or a pharmaceutically acceptable salt thereof:

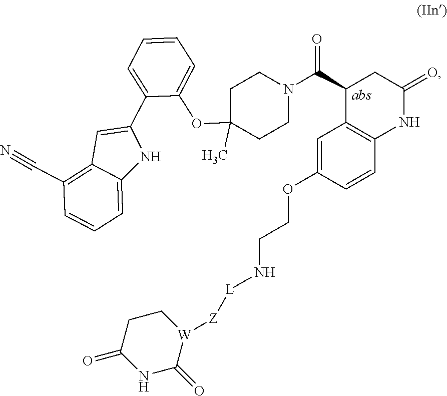

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIo), or a stereoisomer and/or a pharmaceutically acceptable salt thereof:

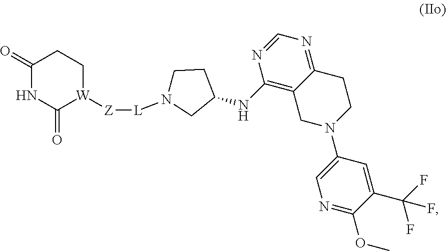

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIp), or a stereoisomer and/or a pharmaceutically acceptable salt thereof:

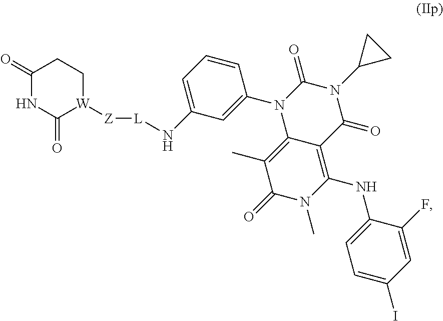

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIq), or a stereoisomer and/or a pharmaceutically acceptable salt thereof:

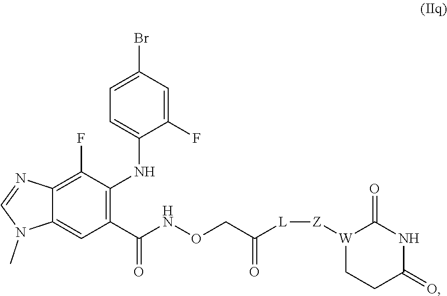

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIr), or a stereoisomer and/or a pharmaceutically acceptable salt thereof:

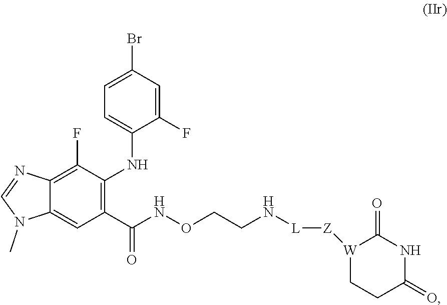

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIs), or a stereoisomer and/or a pharmaceutically acceptable salt thereof:

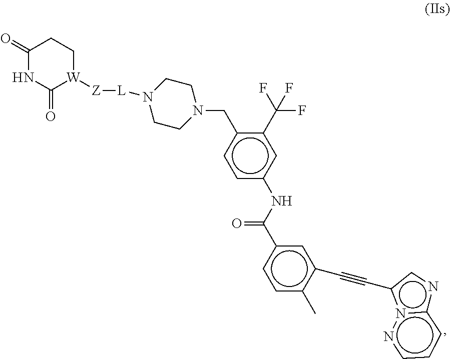

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIt), or a stereoisomer and/or a pharmaceutically acceptable salt thereof:

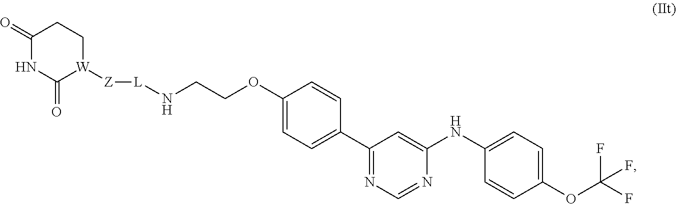

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIu), or a stereoisomer and/or a pharmaceutically acceptable salt thereof:

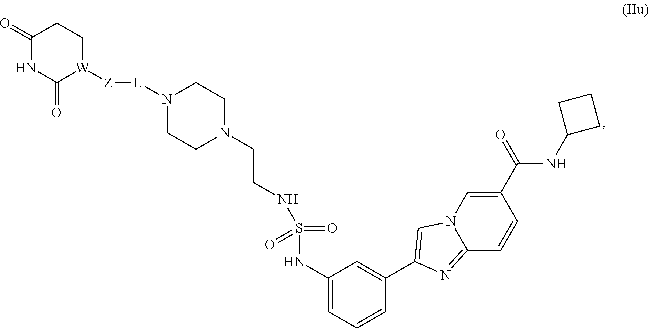

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIv), or a stereoisomer and/or a pharmaceutically acceptable salt thereof:

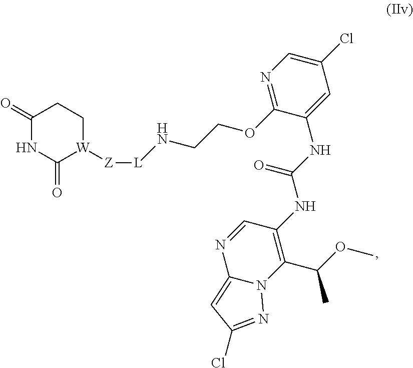

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIw), or a stereoisomer and/or a pharmaceutically acceptable salt thereof:

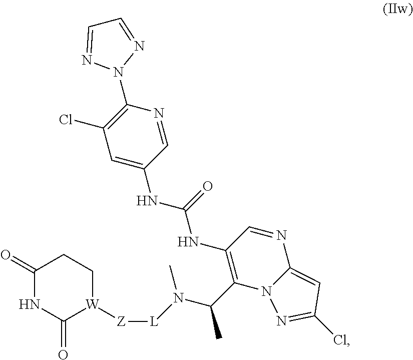

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIx), or a stereoisomer and/or a pharmaceutically acceptable salt thereof:

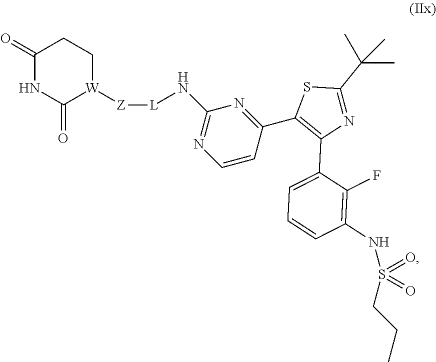

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIy), or a stereoisomer and/or a pharmaceutically acceptable salt thereof:

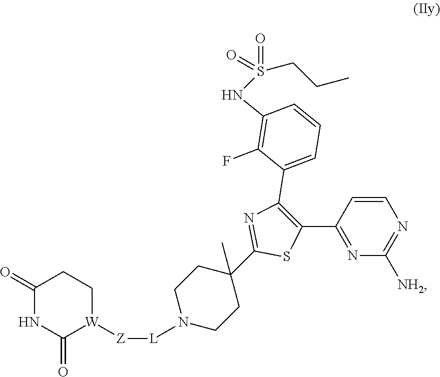

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIz), or a stereoisomer and/or a pharmaceutically acceptable salt thereof:

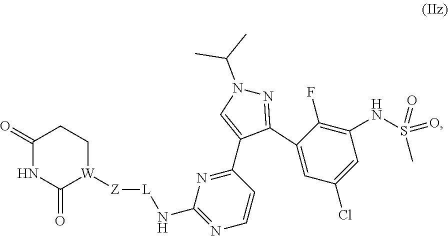

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIaa), or a stereoisomer and/or a pharmaceutically acceptable salt thereof:

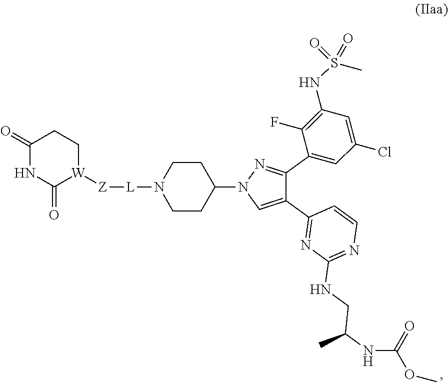

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIbb), or a stereoisomer and/or a pharmaceutically acceptable salt thereof:

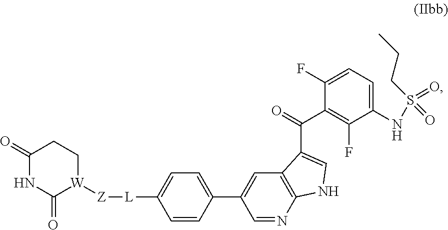

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIcc), or a stereoisomer and/or a pharmaceutically acceptable salt thereof:

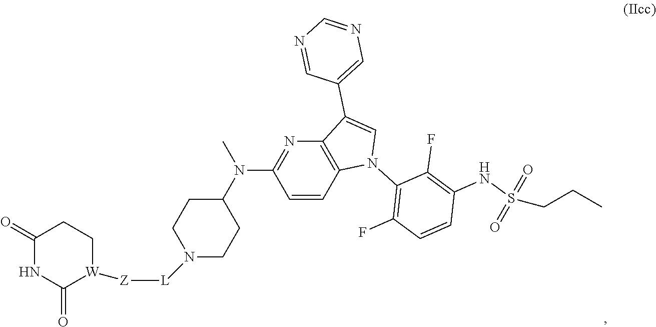

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIdd), or a stereoisomer and/or a pharmaceutically acceptable salt thereof:

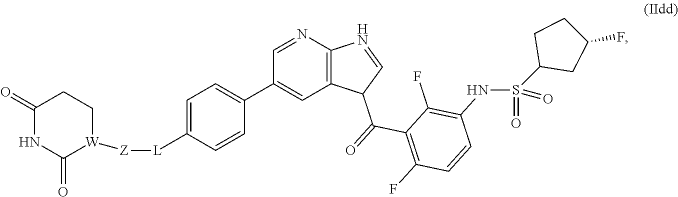

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIee), or a stereoisomer and/or a pharmaceutically acceptable salt thereof:

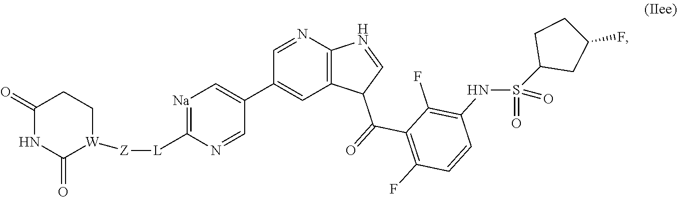

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIff), or a stereoisomer and/or a pharmaceutically acceptable salt thereof:

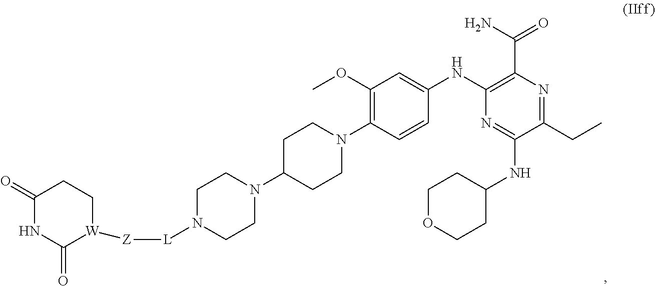

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIgg), or a stereoisomer and/or a pharmaceutically acceptable salt thereof:

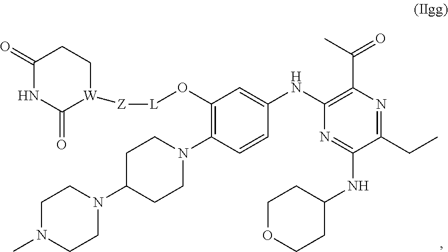

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIhh), or a stereoisomer and/or a pharmaceutically acceptable salt thereof:

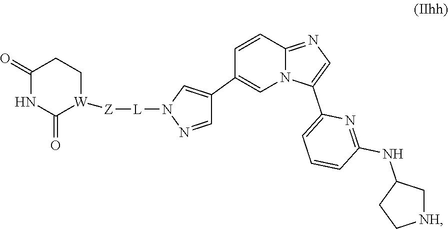

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIii), or a stereoisomer and/or a pharmaceutically acceptable salt thereof:

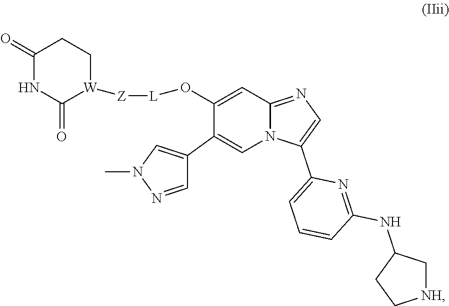

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIjj), or a stereoisomer and/or a pharmaceutically acceptable salt thereof:

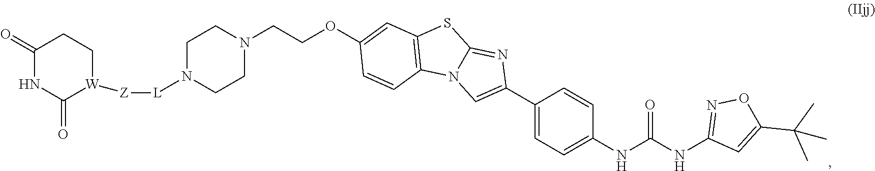

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIkk), or a stereoisomer and/or a pharmaceutically acceptable salt thereof:

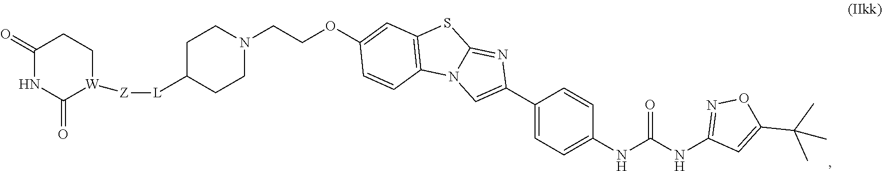

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IImm), or a stereoisomer and/or a pharmaceutically acceptable salt thereof:

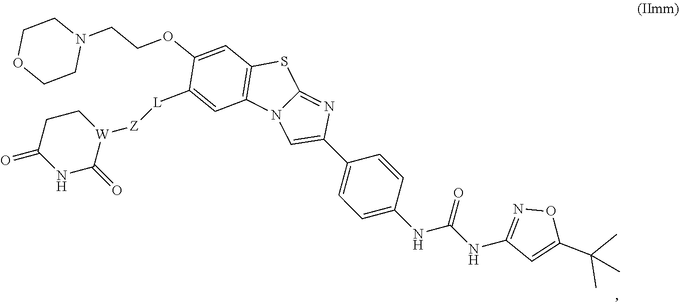

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IInn), or a stereoisomer and/or a pharmaceutically acceptable salt thereof:

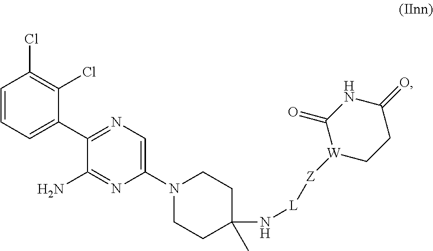

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIoo), or a stereoisomer and/or a pharmaceutically acceptable salt thereof:

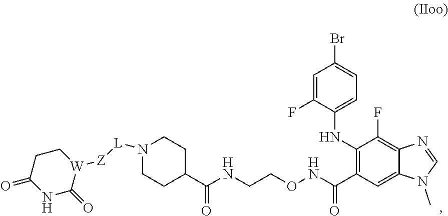

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIpp), or a stereoisomer and/or a pharmaceutically acceptable salt thereof:

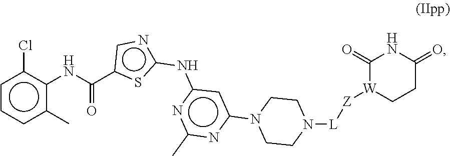

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIqq), or a stereoisomer and/or a pharmaceutically acceptable salt thereof:

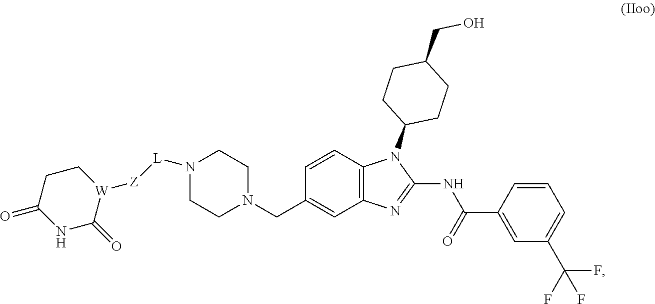

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIqq), or a stereoisomer and/or a pharmaceutically acceptable salt thereof:

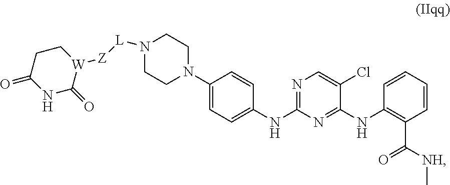

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds of Formula (IIrr), or a stereoisomer and/or a pharmaceutically acceptable salt thereof:

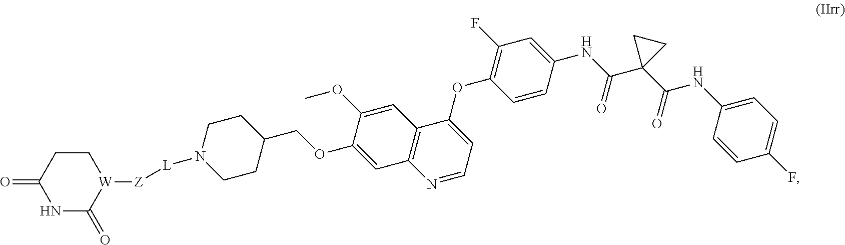

wherein the variables are as described below. In certain embodiments, the left side of the compound is a harness, and the harness is any harness described herein. In certain embodiments, the portion of the compound bonded to the left of L is a harness, and the harness is any harness described herein. In certain embodiments, the harness is any of Formulas A-β, in the table below. In certain embodiments, the harness is any of Formulas AA-ββ, in the table below. In certain embodiments, the harness is according to Formula A. In certain embodiments, the harness is according to Formula B. In certain embodiments, the harness is according to Formula C. In certain embodiments, the harness is according to Formula D. In certain embodiments, the harness is according to Formula E. In certain embodiments, the harness is according to Formula F. In certain embodiments, the harness is according to Formula G. In certain embodiments, the harness is according to Formula H. In certain embodiments, the harness is according to Formula I. In certain embodiments, the harness is according to Formula J. In certain embodiments, the harness is according to Formula K. In certain embodiments, the harness is according to Formula L. In certain embodiments, the harness is according to Formula M. In certain embodiments, the harness is according to Formula N. In certain embodiments, the harness is according to Formula O. In certain embodiments, the harness is according to Formula P. In certain embodiments, the harness is according to Formula Q. In certain embodiments, the harness is according to Formula R. In certain embodiments, the harness is according to Formula S. In certain embodiments, the harness is according to Formula T. In certain embodiments, the harness is according to Formula U. In certain embodiments, the harness is according to Formula V. In certain embodiments, the harness is according to Formula W. In certain embodiments, the harness is according to Formula X. In certain embodiments, the harness is according to Formula Y. In certain embodiments, the harness is according to Formula Z. In certain embodiments, the harness is according to Formula α. In certain embodiments, the harness is according to Formula β. In certain embodiments, the harness is according to Formula AA. In certain embodiments, the harness is according to Formula BB. In certain embodiments, the harness is according to Formula CC. In certain embodiments, the harness is according to Formula DD. In certain embodiments, the harness is according to Formula EE. In certain embodiments, the harness is according to Formula FF. In certain embodiments, the harness is according to Formula GG. In certain embodiments, the harness is according to Formula HH. In certain embodiments, the harness is according to Formula II. In certain embodiments, the harness is according to Formula JJ. In certain embodiments, the harness is according to Formula KK. In certain embodiments, the harness is according to Formula LL. In certain embodiments, the harness is according to Formula MM. In certain embodiments, the harness is according to Formula NN. In certain embodiments, the harness is according to Formula OO. In certain embodiments, the harness is according to Formula PP. In certain embodiments, the harness is according to Formula QQ. In certain embodiments, the harness is according to Formula RR. In certain embodiments, the harness is according to Formula SS. In certain embodiments, the harness is according to Formula TT. In certain embodiments, the harness is according to Formula UU. In certain embodiments, the harness is according to Formula VV. In certain embodiments, the harness is according to Formula WW. In certain embodiments, the harness is according to Formula XX. In certain embodiments, the harness is according to Formula YY. In certain embodiments, the harness is according to Formula ZZ. In certain embodiments, the harness is according to Formula αα. In certain embodiments, the harness is according to Formula ββ.

In certain embodiments, provided herein are compounds comprising any of the following harnesses:

| Formula | Harness | Harness | Formula |
|---|---|---|---|
| A | 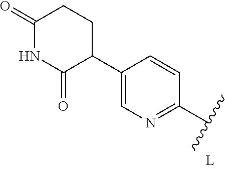 [SMILES: *c1ccc(C2CCC(=O)NC2=O)cn1] | 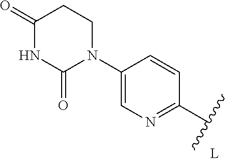 [SMILES: *c1ccc(N2CCC(=O)NC2=O)cn1] | AA |
| B | 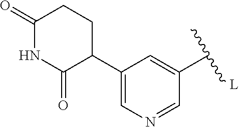 [SMILES: *c1cncc(C2CCC(=O)NC2=O)c1] | 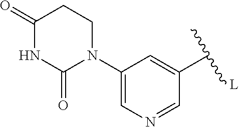 [SMILES: *c1cncc(N2CCC(=O)NC2=O)c1] | BB |
| C | 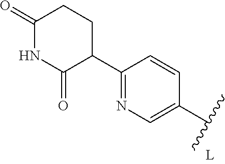 [SMILES: *c1ccc(C2CCC(=O)NC2=O)nc1] | 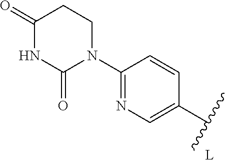 [SMILES: *c1ccc(N2CCC(=O)NC2=O)nc1] | CC |
| D | 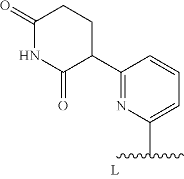 [SMILES: *c1cccc(C2CCC(=O)NC2=O)n1] | 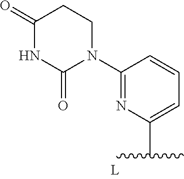 [SMILES: *c1cccc(N2CCC(=O)NC2=O)n1] | DD |
| E | 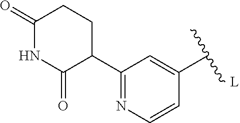 [SMILES: *c1ccnc(C2CCC(=O)NC2=O)c1] | 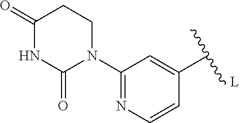 [SMILES: *c1ccnc(N2CCC(=O)NC2=O)c1] | EE |
| F | 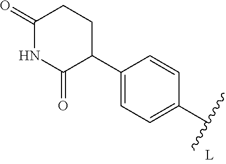 [SMILES: *c1ccc(C2CCC(=O)NC2=O)cc1] | 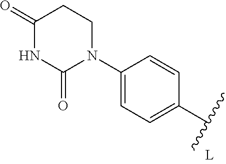 [SMILES: *c1ccc(N2CCC(=O)NC2=O)cc1] | FF |
| G | 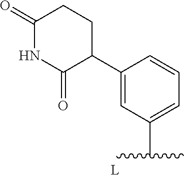 [SMILES: *c1cccc(C2CCC(=O)NC2=O)c1] | 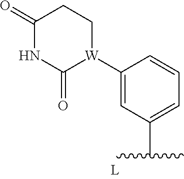 | GG |
| H | 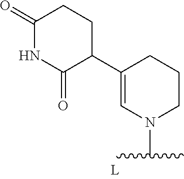 [SMILES: *N1C=C(C2CCC(=O)NC2=O)CCC1] | 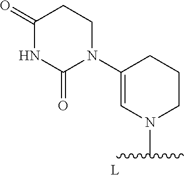 [SMILES: *N1C=C(N2CCC(=O)NC2=O)CCC1] | HH |
| I | 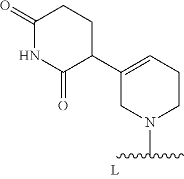 [SMILES: *N1CCC=C(C2CCC(=O)NC2=O)C1] | 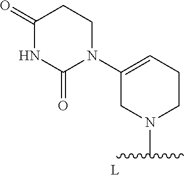 [SMILES: *N1CCC=C(N2CCC(=O)NC2=O)C1] | II |
| J | 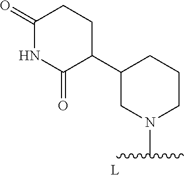 [SMILES: *N1CCCC(C2CCC(=O)NC2=O)C1] | 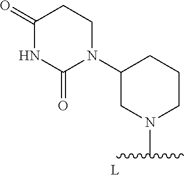 [SMILES: *N1CCCC(N2CCC(=O)NC2=O)C1] | JJ |
| K | 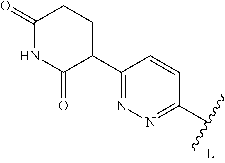 [SMILES: *c1ccc(C2CCC(=O)NC2=O)nn1] | 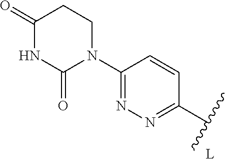 [SMILES: *c1ccc(N2CCC(=O)NC2=O)nn1] | KK |
| L | 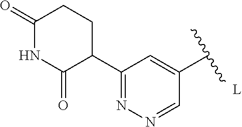 [SMILES: *c1cnnc(C2CCC(=O)NC2=O)c1] | 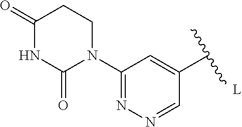 [SMILES: *c1cnnc(N2CCC(=O)NC2=O)c1] | LL |
| M | 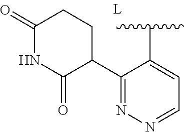 [SMILES: *c1ccnnc1C1CCC(=O)NC1=O] | 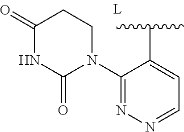 [SMILES: *c1ccnnc1N1CCC(=O)NC1=O] | MM |
| N | 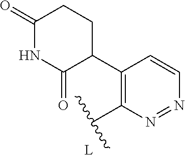 [SMILES: *c1nnccc1C1CCC(=O)NC1=O] | 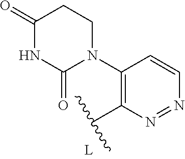 [SMILES: *c1nnccc1N1CCC(=O)NC1=O] | NN |
| O | 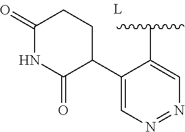 [SMILES: *c1cnncc1C1CCC(=O)NC1=O] | 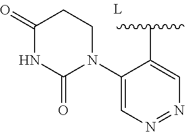 [SMILES: *c1cnncc1N1CCC(=O)NC1=O] | OO |
| P | 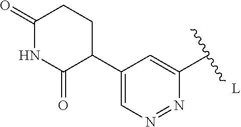 [SMILES: *c1cc(C2CCC(=O)NC2=O)cnn1] | 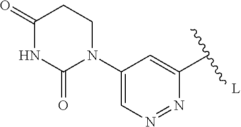 [SMILES: *c1cc(N2CCC(=O)NC2=O)cnn1] | PP |
| Q | 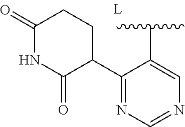 [SMILES: *c1cncnc1C1CCC(=O)NC1=O] | 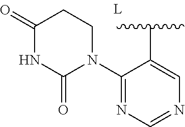 [SMILES: *c1cncnc1N1CCC(=O)NC1=O] | QQ |
| R | 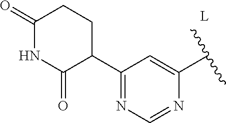 [SMILES: *c1cc(C2CCC(=O)NC2=O)ncn1] | 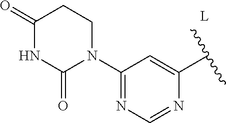 [SMILES: *c1cc(N2CCC(=O)NC2=O)ncn1] | RR |
| S | 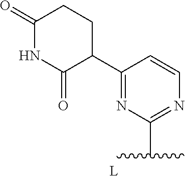 [SMILES: *c1nccc(C2CCC(=O)NC2=O)n1] | 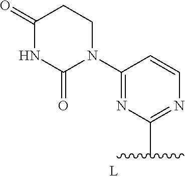 [SMILES: *c1nccc(N2CCC(=O)NC2=O)n1] | SS |
| T | 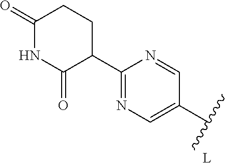 [SMILES: *c1cnc(C2CCC(=O)NC2=O)nc1] | 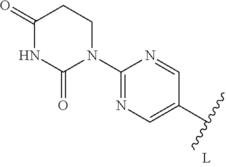 [SMILES: *c1cnc(N2CCC(=O)NC2=O)nc1] | TT |
| U | 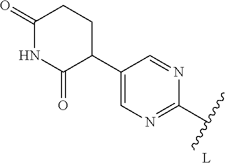 [SMILES: *c1ncc(C2CCC(=O)NC2=O)cn1] | 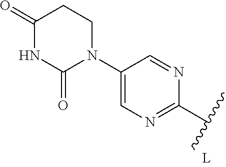 [SMILES: *c1ncc(N2CCC(=O)NC2=O)cn1] | UU |
| V | 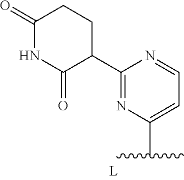 [SMILES: *c1ccnc(C2CCC(=O)NC2=O)n1] | 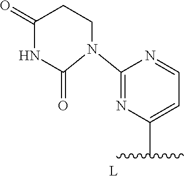 [SMILES: *c1ccnc(N2CCC(=O)NC2=O)n1] | VV |
| W | 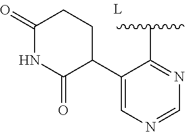 [SMILES: *c1ncncc1C1CCC(=O)NC1=O] | 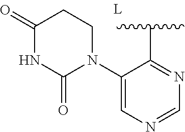 [SMILES: *c1ncncc1N1CCC(=O)NC1=O] | WW |
| X | 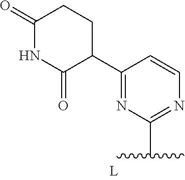 [SMILES: *c1nccc(C2CCC(=O)NC2=O)n1] | 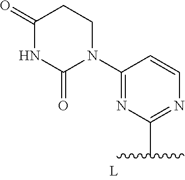 [SMILES: *c1nccc(N2CCC(=O)NC2=O)n1] | XX |
| Y | 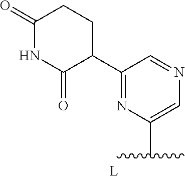 [SMILES: *c1cncc(C2CCC(=O)NC2=O)n1] | 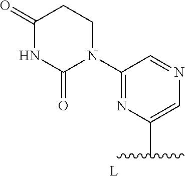 [SMILES: *c1cncc(N2CCC(=O)NC2=O)n1] | YY |
| Z | 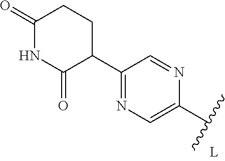 [SMILES: *c1cnc(C2CCC(=O)NC2=O)cn1] | 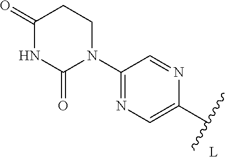 [SMILES: *c1cnc(N2CCC(=O)NC2=O)cn1] | ZZ |
| α | 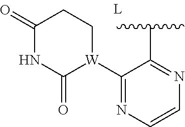 | 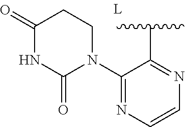 [SMILES: *c1nccnc1N1CCC(=O)NC1=O] | αα |
| β | 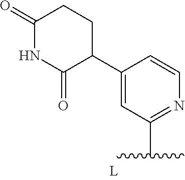 [SMILES: *c1cc(C2CCC(=O)NC2=O)ccn1] | 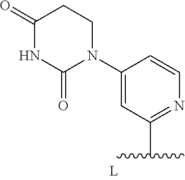 [SMILES: *c1cc(N2CCC(=O)NC2=O)ccn1] | ββ |

In each harness, the wiggle line and L indicate the bond to L and the remainder of the compound.

In certain embodiments, provided herein are compounds of Formula (I1), or a stereoisomer and/or a pharmaceutically acceptable salt thereof:

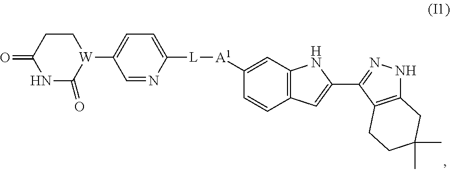

wherein the variables are as described below.

In certain embodiments, provided herein are compounds of Formula (12), or a stereoisomer and/or a pharmaceutically acceptable salt thereof:

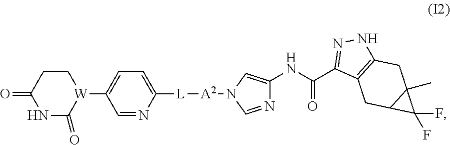

wherein the variables are as described below.

In certain embodiments, provided herein are compounds of Formula (I2A), or a stereoisomer and/or a pharmaceutically acceptable salt thereof:

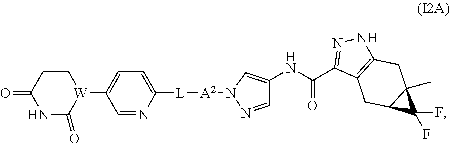

wherein the variables are as described below.

In certain embodiments, provided herein are compounds of Formula (I3), or a stereoisomer and/or a pharmaceutically acceptable salt thereof:

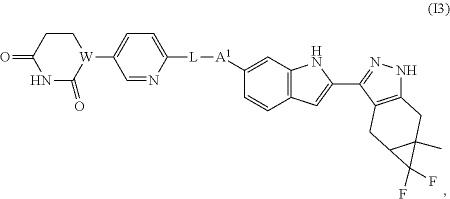

wherein the variables are as described below.

In certain embodiments, provided herein are compounds of Formula (I3A), or a stereoisomer and/or a pharmaceutically acceptable salt thereof:

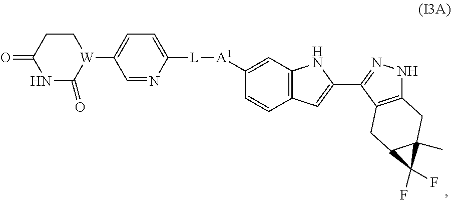

wherein the variables are as described below.

In certain embodiments, provided herein are compounds of Formula (14), or a stereoisomer and/or a pharmaceutically acceptable salt thereof:

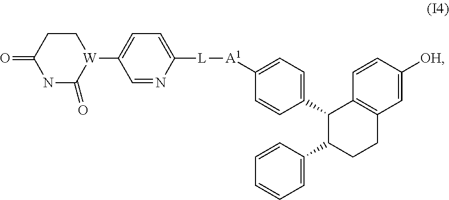

wherein the variables are as described below.

In certain embodiments, provided herein are compounds of Formula (15), or a stereoisomer and/or a pharmaceutically acceptable salt thereof:

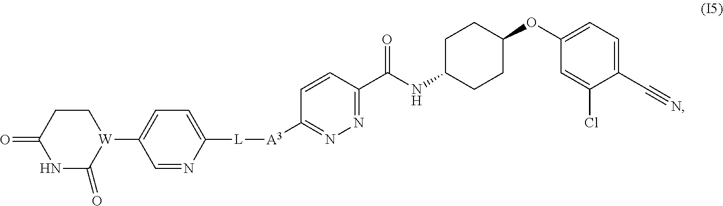

wherein the variables are as described below.

In certain embodiments, provided herein are compounds of Formula (16), or a stereoisomer and/or a pharmaceutically acceptable salt thereof:

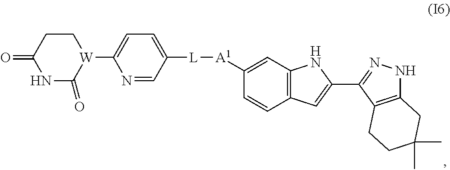

wherein the variables are as described below.

In certain embodiments, provided herein are compounds of Formula (I7), or a stereoisomer and/or a pharmaceutically acceptable salt thereof:

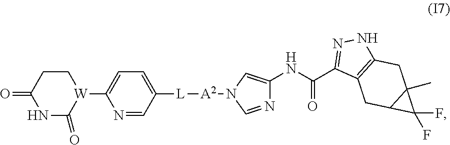

wherein the variables are as described below.

In certain embodiments, provided herein are compounds of Formula (I7A), or a stereoisomer and/or a pharmaceutically acceptable salt thereof:

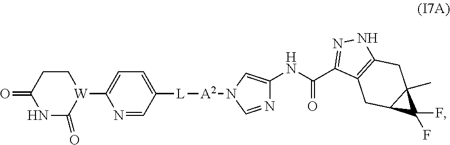

wherein the variables are as described below.

In certain embodiments, provided herein are compounds of Formula (I8), or a stereoisomer and/or a pharmaceutically acceptable salt thereof:

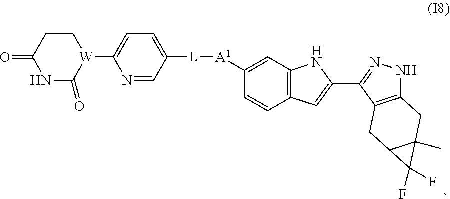

wherein the variables are as described below.

In certain embodiments, provided herein are compounds of Formula (I8A), or a stereoisomer and/or a pharmaceutically acceptable salt thereof:

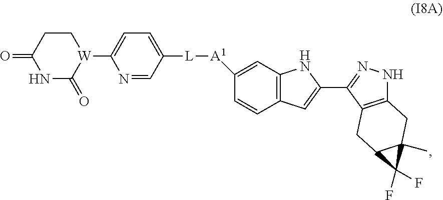

wherein the variables are as described below.

In certain embodiments, provided herein are compounds of Formula (I9), or a stereoisomer and/or a pharmaceutically acceptable salt thereof:

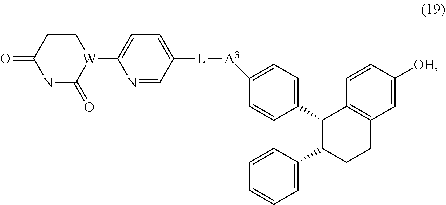

wherein the variables are as described below.

In certain embodiments, provided herein are compounds of Formula (I10), or a stereoisomer and/or a pharmaceutically acceptable salt thereof:

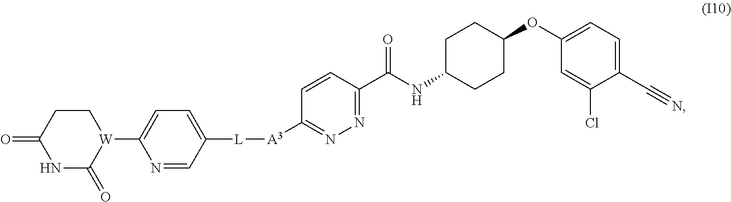

wherein the variables are as described below.

In certain embodiments, provided herein are compounds of Formula (I11), or a stereoisomer and/or a pharmaceutically acceptable salt thereof:

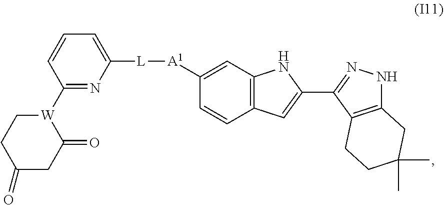

wherein the variables are as described below.

In certain embodiments, provided herein are compounds of Formula (I12), or a stereoisomer and/or a pharmaceutically acceptable salt thereof:

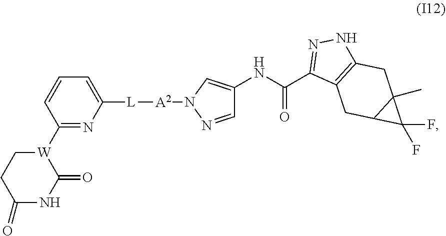

wherein the variables are as described below.

In certain embodiments, provided herein are compounds of Formula (I12A), or a stereoisomer and/or a pharmaceutically acceptable salt thereof:

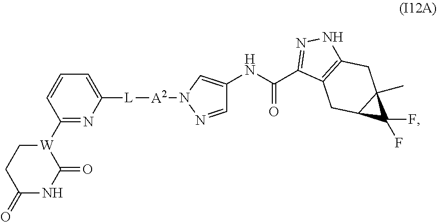

wherein the variables are as described below.

In certain embodiments, provided herein are compounds of Formula (I13), or a stereoisomer and/or a pharmaceutically acceptable salt thereof:

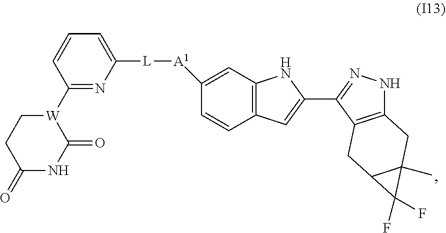

wherein the variables are as described below.

In certain embodiments, provided herein are compounds of Formula (I13A), or a stereoisomer and/or a pharmaceutically acceptable salt thereof:

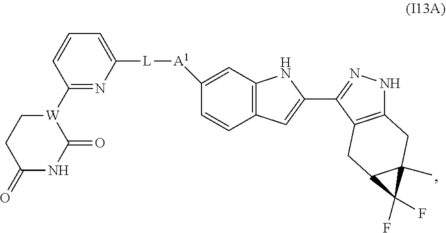

wherein the variables are as described below.

In certain embodiments, provided herein are compounds of Formula (I14), or a stereoisomer and/or a pharmaceutically acceptable salt thereof:

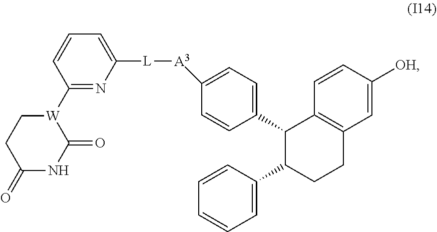

wherein the variables are as described below.

In certain embodiments, provided herein are compounds of Formula (I15), or a stereoisomer and/or a pharmaceutically acceptable salt thereof:

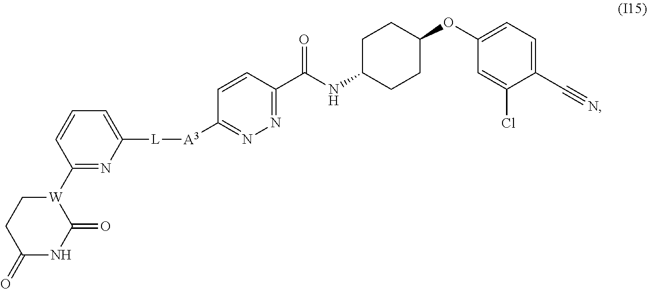

wherein the variables are as described below.

In certain embodiments, provided herein are compounds of Formula (I16), or a stereoisomer and/or a pharmaceutically acceptable salt thereof:

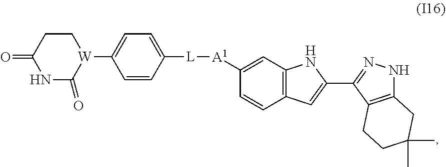

wherein the variables are as described below.

In certain embodiments, provided herein are compounds of Formula (I17), or a stereoisomer and/or a pharmaceutically acceptable salt thereof:

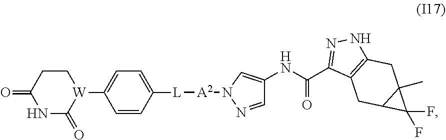

wherein the variables are as described below.

In certain embodiments, provided herein are compounds of Formula (I17A), or a stereoisomer and/or a pharmaceutically acceptable salt thereof:

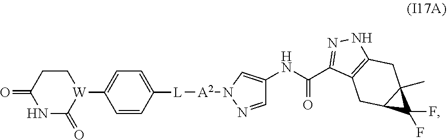

wherein the variables are as described below.

In certain embodiments, provided herein are compounds of Formula (I18), or a stereoisomer and/or a pharmaceutically acceptable salt thereof:

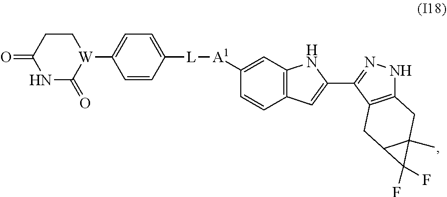

wherein the variables are as described below.

In certain embodiments, provided herein are compounds of Formula (I18A), or a stereoisomer and/or a pharmaceutically acceptable salt thereof:

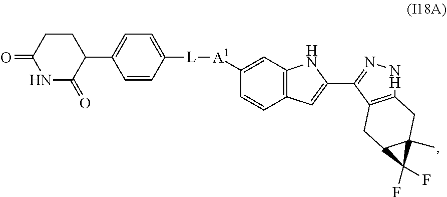

wherein the variables are as described below.

In certain embodiments, provided herein are compounds of Formula (I19), or a stereoisomer and/or a pharmaceutically acceptable salt thereof:

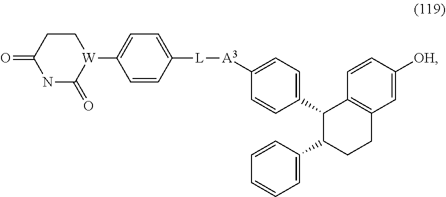

wherein the variables are as described below.

In certain embodiments, provided herein are compounds of Formula (I20), or a stereoisomer and/or a pharmaceutically acceptable salt thereof:

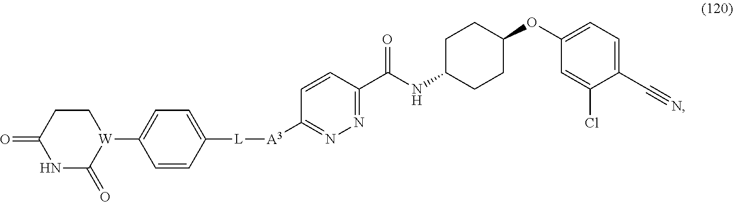

wherein the variables are as described below.

In certain embodiments, provided herein are compounds of Formula (I21), or a stereoisomer and/or a pharmaceutically acceptable salt thereof:

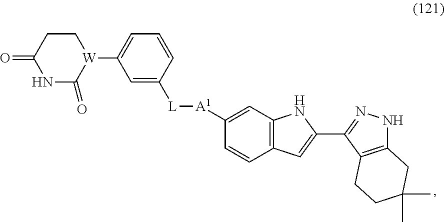

wherein the variables are as described below.

In certain embodiments, provided herein are compounds of Formula (I22), or a stereoisomer and/or a pharmaceutically acceptable salt thereof:

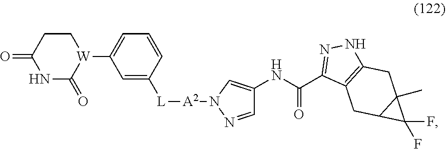

wherein the variables are as described below.

In certain embodiments, provided herein are compounds of Formula (I22A), or a stereoisomer and/or a pharmaceutically acceptable salt thereof:

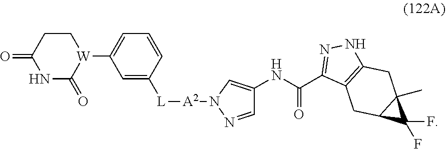

In certain embodiments, provided herein are compounds of Formula (I23), or a stereoisomer and/or a pharmaceutically acceptable salt thereof:

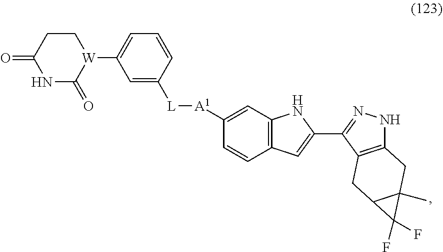

wherein the variables are as described below.

In certain embodiments, provided herein are compounds of Formula (I23A), or a stereoisomer and/or a pharmaceutically acceptable salt thereof:

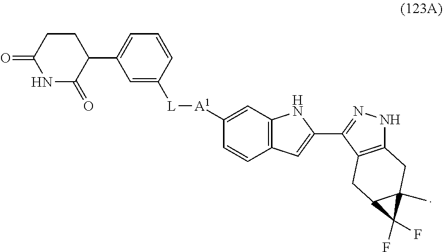

In certain embodiments, provided herein are compounds of Formula (I24), or a stereoisomer and/or a pharmaceutically acceptable salt thereof:

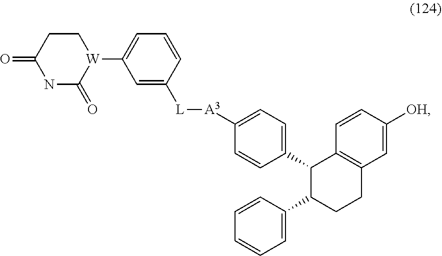

wherein the variables are as described below.

In certain embodiments, provided herein are compounds of Formula (I25), or a stereoisomer and/or a pharmaceutically acceptable salt thereof:

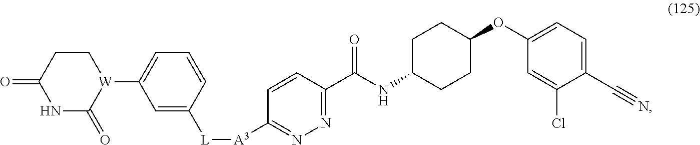

wherein the variables are as described below.

In certain embodiments, provided herein are compounds of Formula (I26), or a stereoisomer and/or a pharmaceutically acceptable salt thereof:

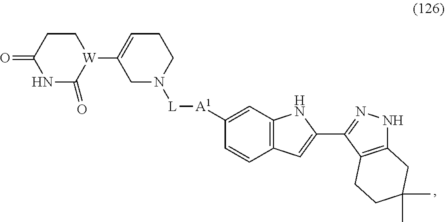

wherein the variables are as described below.

In certain embodiments, provided herein are compounds of Formula (I27), or a stereoisomer and/or a pharmaceutically acceptable salt thereof:

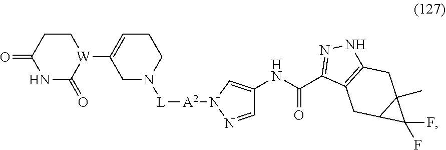

wherein the variables are as described below.

In certain embodiments, provided herein are compounds of Formula (I27A), or a stereoisomer and/or a pharmaceutically acceptable salt thereof:

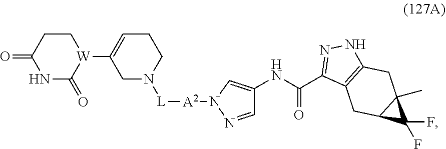

wherein the variables are as described below.

In certain embodiments, provided herein are compounds of Formula (I28), or a stereoisomer and/or a pharmaceutically acceptable salt thereof:

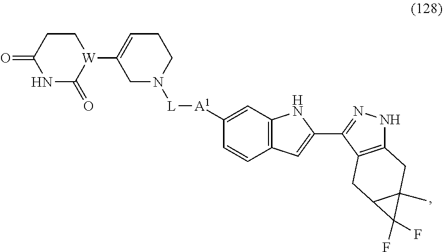

wherein the variables are as described below.

In certain embodiments, provided herein are compounds of Formula (I28A), or a stereoisomer and/or a pharmaceutically acceptable salt thereof:

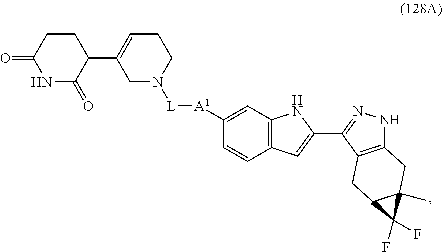

wherein the variables are as described below.

In certain embodiments, provided herein are compounds of Formula (I29), or a stereoisomer and/or a pharmaceutically acceptable salt thereof:

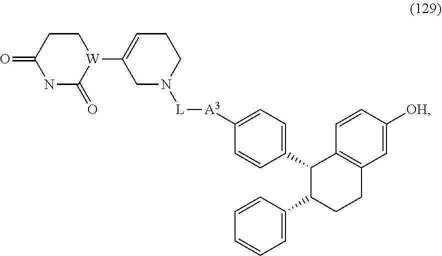

wherein the variables are as described below.

In certain embodiments, provided herein are compounds of Formula (I30), or a stereoisomer and/or a pharmaceutically acceptable salt thereof:

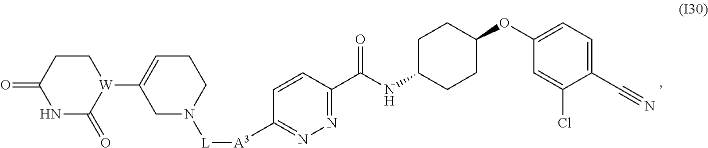

wherein the variables are as described below.

In certain embodiments, provided herein are compounds of Formula (I31), or a stereoisomer and/or a pharmaceutically acceptable salt thereof:

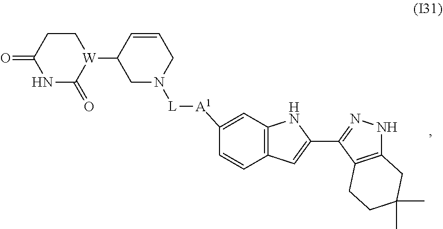

wherein the variables are as described below.

In certain embodiments, provided herein are compounds of Formula (I32), or a stereoisomer and/or a pharmaceutically acceptable salt thereof:

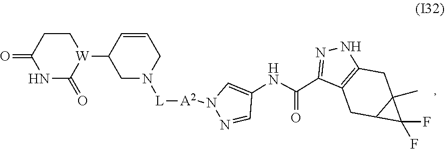

wherein the variables are as described below.

In certain embodiments, provided herein are compounds of Formula (I32A), or a stereoisomer and/or a pharmaceutically acceptable salt thereof:

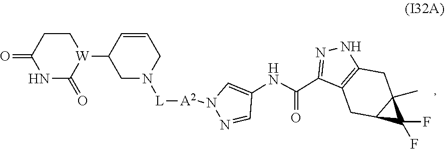

wherein the variables are as described below.

In certain embodiments, provided herein are compounds of Formula (I33), or a stereoisomer and/or a pharmaceutically acceptable salt thereof:

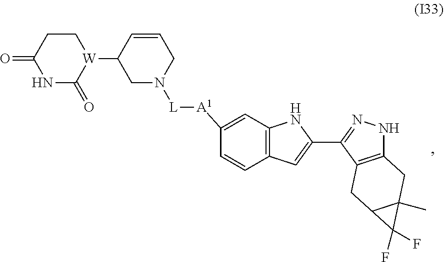

wherein the variables are as described below.

In certain embodiments, provided herein are compounds of Formula (I33A), or a stereoisomer and/or a pharmaceutically acceptable salt thereof:

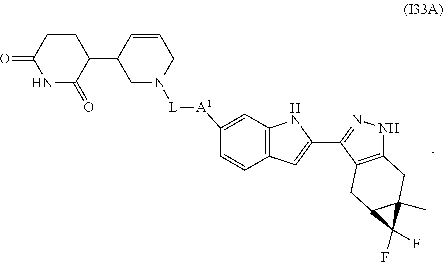

In certain embodiments, provided herein are compounds of Formula (I34), or a stereoisomer and/or a pharmaceutically acceptable salt thereof:

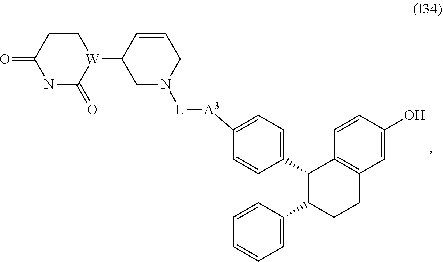

wherein the variables are as described below.

In certain embodiments, provided herein are compounds of Formula (I35), or a stereoisomer and/or a pharmaceutically acceptable salt thereof:

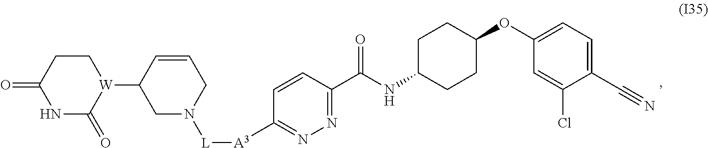

wherein the variables are as described below.

In certain embodiments, provided herein are compounds of Formula (I36), or a stereoisomer and/or a pharmaceutically acceptable salt thereof:

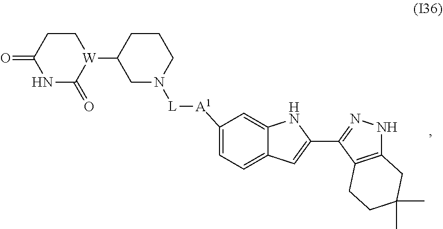

wherein the variables are as described below.

In certain embodiments, provided herein are compounds of Formula (I37), or a stereoisomer and/or a pharmaceutically acceptable salt thereof:

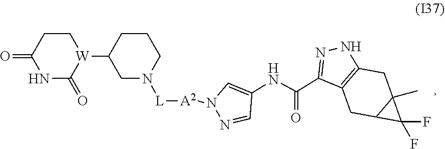

wherein the variables are as described below.

In certain embodiments, provided herein are compounds of Formula (I37A), or a stereoisomer and/or a pharmaceutically acceptable salt thereof:

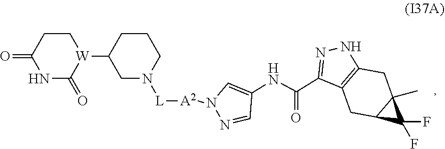

wherein the variables are as described below.

In certain embodiments, provided herein are compounds of Formula (I38), or a stereoisomer and/or a pharmaceutically acceptable salt thereof:

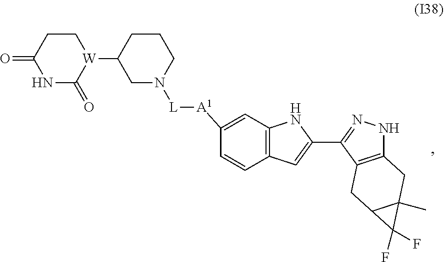

wherein the variables are as described below.

In certain embodiments, provided herein are compounds of Formula (I38A), or a stereoisomer and/or a pharmaceutically acceptable salt thereof:

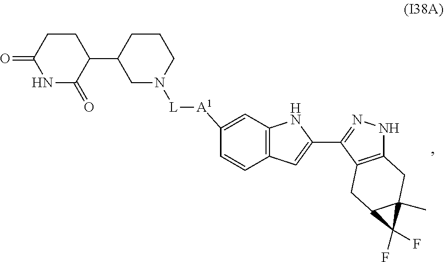

wherein the variables are as described below.

In certain embodiments, provided herein are compounds of Formula (I39), or a stereoisomer and/or a pharmaceutically acceptable salt thereof:

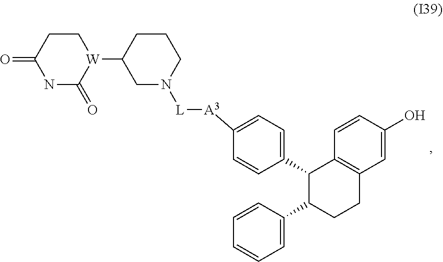

wherein the variables are as described below.

In certain embodiments, provided herein are compounds of Formula (I40), or a stereoisomer and/or a pharmaceutically acceptable salt thereof:

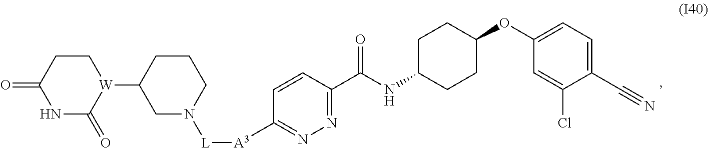

wherein the variables are as described below.

In certain embodiments, provided herein are compounds of Formula (I41), or a stereoisomer and/or a pharmaceutically acceptable salt thereof:

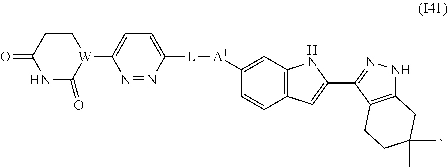

wherein the variables are as described below.

In certain embodiments, provided herein are compounds of Formula (I42), or a stereoisomer and/or a pharmaceutically acceptable salt thereof:

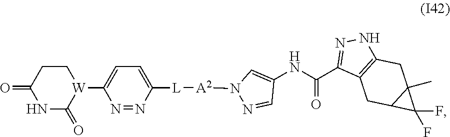

wherein the variables are as described below.

In certain embodiments, provided herein are compounds of Formula (I42A), or a stereoisomer and/or a pharmaceutically acceptable salt thereof:

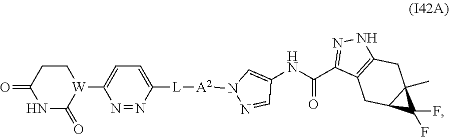

wherein the variables are as described below.

In certain embodiments, provided herein are compounds of Formula (I43), or a stereoisomer and/or a pharmaceutically acceptable salt thereof:

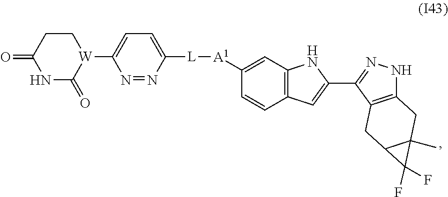

wherein the variables are as described below.

In certain embodiments, provided herein are compounds of Formula (I43A), or a stereoisomer and/or a pharmaceutically acceptable salt thereof:

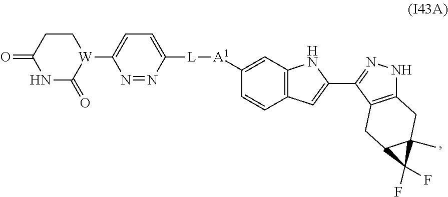

wherein the variables are as described below.

In certain embodiments, provided herein are compounds of Formula (I44), or a stereoisomer and/or a pharmaceutically acceptable salt thereof:

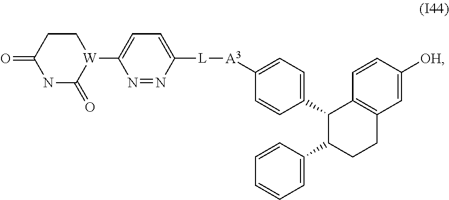

wherein the variables are as described below.

In certain embodiments, provided herein are compounds of Formula (I45), or a stereoisomer and/or a pharmaceutically acceptable salt thereof:

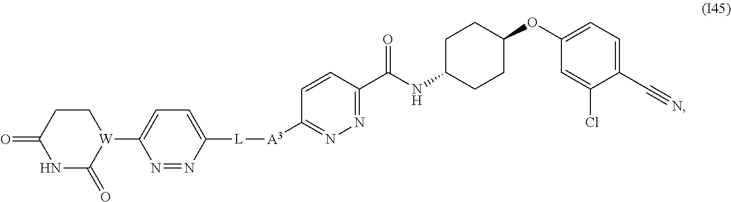

wherein the variables are as described below.

In certain embodiments, provided herein are compounds of Formula (I46), or a stereoisomer and/or a pharmaceutically acceptable salt thereof:

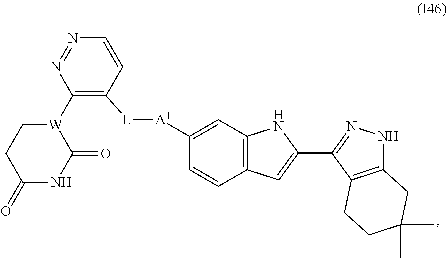

wherein the variables are as described below.

In certain embodiments, provided herein are compounds of Formula (I47), or a stereoisomer and/or a pharmaceutically acceptable salt thereof:

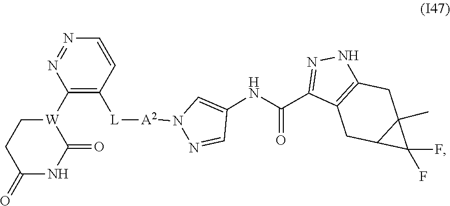

wherein the variables are as described below.

In certain embodiments, provided herein are compounds of Formula (I47A), or a stereoisomer and/or a pharmaceutically acceptable salt thereof:

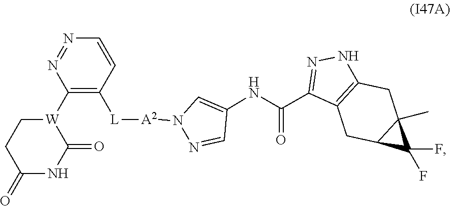

wherein the variables are as described below.

In certain embodiments, provided herein are compounds of Formula (I48), or a stereoisomer and/or a pharmaceutically acceptable salt thereof:

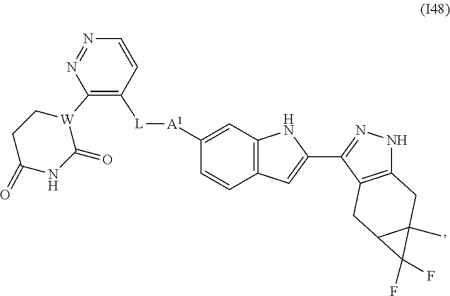

wherein the variables are as described below.

In certain embodiments, provided herein are compounds of Formula (I48A), or a stereoisomer and/or a pharmaceutically acceptable salt thereof:

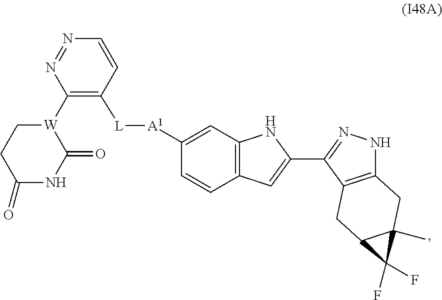

wherein the variables are as described below.

In certain embodiments, provided herein are compounds of Formula (I49), or a stereoisomer and/or a pharmaceutically acceptable salt thereof:

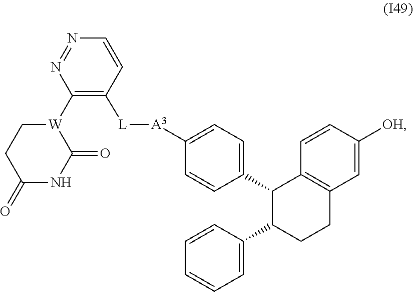

wherein the variables are as described below.

In certain embodiments, provided herein are compounds of Formula (I50), or a stereoisomer and/or a pharmaceutically acceptable salt thereof:

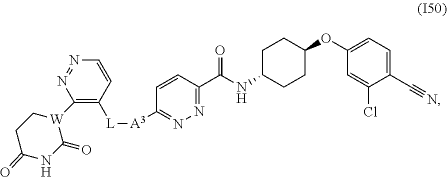

wherein the variables are as described below.

In certain embodiments, provided herein are compounds of Formula (I51), or a stereoisomer and/or a pharmaceutically acceptable salt thereof:

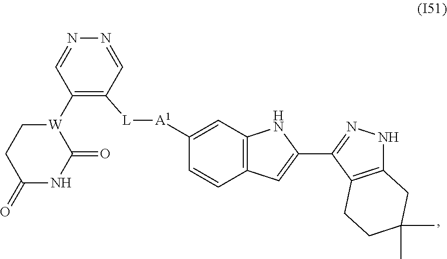

wherein the variables are as described below.

In certain embodiments, provided herein are compounds of Formula (I52), or a stereoisomer and/or a pharmaceutically acceptable salt thereof:

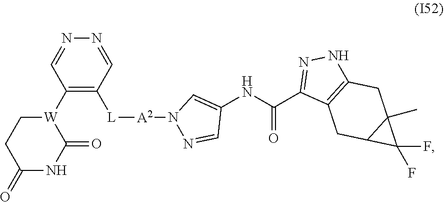

wherein the variables are as described below.

In certain embodiments, provided herein are compounds of Formula (I52A), or a stereoisomer and/or a pharmaceutically acceptable salt thereof:

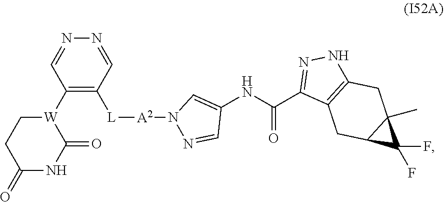

wherein the variables are as described below.

In certain embodiments, provided herein are compounds of Formula (I53), or a stereoisomer and/or a pharmaceutically acceptable salt thereof:

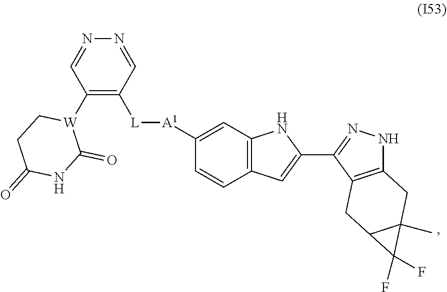

wherein the variables are as described below.

In certain embodiments, provided herein are compounds of Formula (I53A), or a stereoisomer and/or a pharmaceutically acceptable salt thereof:

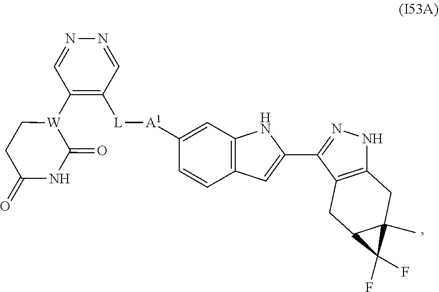

wherein the variables are as described below.

In certain embodiments, provided herein are compounds of Formula (I54), or a stereoisomer and/or a pharmaceutically acceptable salt thereof:

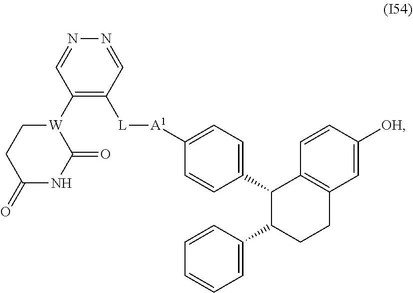

wherein the variables are as described below.

In certain embodiments, provided herein are compounds of Formula (I55), or a stereoisomer and/or a pharmaceutically acceptable salt thereof:

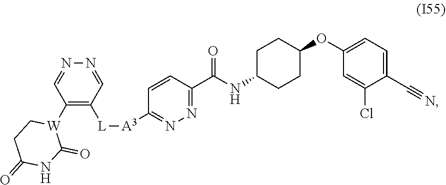

wherein the variables are as described below.

In certain embodiments, provided herein are compounds of Formula (I56), or a stereoisomer and/or a pharmaceutically acceptable salt thereof:

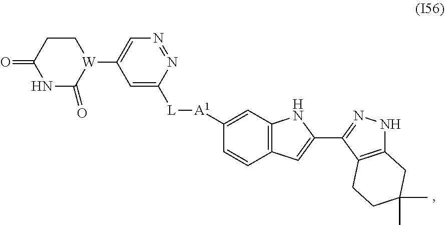

wherein the variables are as described below.

In certain embodiments, provided herein are compounds of Formula (I57), or a stereoisomer and/or a pharmaceutically acceptable salt thereof:

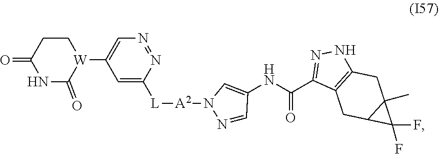

wherein the variables are as described below.

In certain embodiments, provided herein are compounds of Formula (I57A), or a stereoisomer and/or a pharmaceutically acceptable salt thereof:

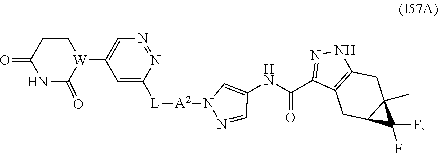

wherein the variables are as described below.

In certain embodiments, provided herein are compounds of Formula (I58), or a stereoisomer and/or a pharmaceutically acceptable salt thereof:

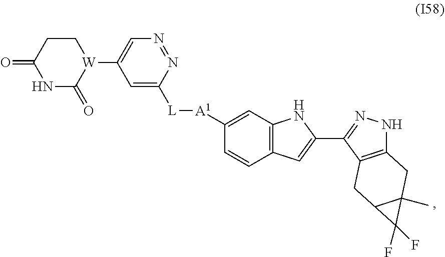

wherein the variables are as described below.

In certain embodiments, provided herein are compounds of Formula (I58A), or a stereoisomer and/or a pharmaceutically acceptable salt thereof:

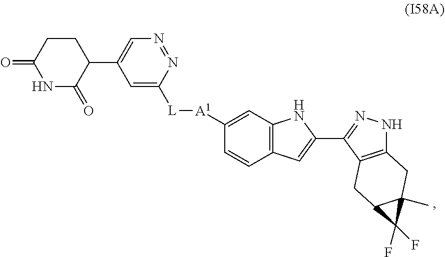

wherein the variables are as described below.

In certain embodiments, provided herein are compounds of Formula (I59), or a stereoisomer and/or a pharmaceutically acceptable salt thereof:

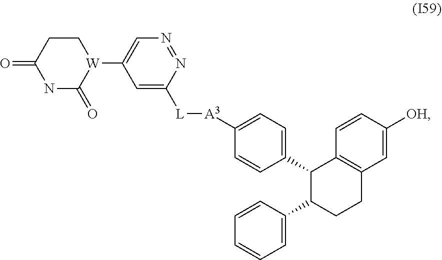

wherein the variables are as described below.

In certain embodiments, provided herein are compounds of Formula (I60), or a stereoisomer and/or a pharmaceutically acceptable salt thereof:

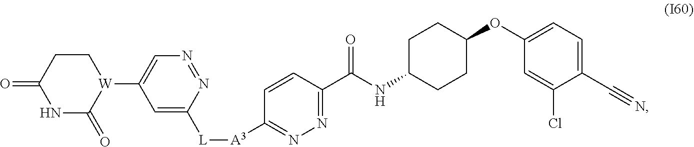

wherein the variables are as described below.

In certain embodiments, provided herein are compounds of Formula (I61), or a stereoisomer and/or a pharmaceutically acceptable salt thereof:

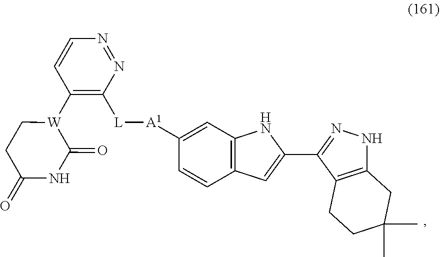

wherein the variables are as described below.

In certain embodiments, provided herein are compounds of Formula (I62), or a stereoisomer and/or a pharmaceutically acceptable salt thereof:

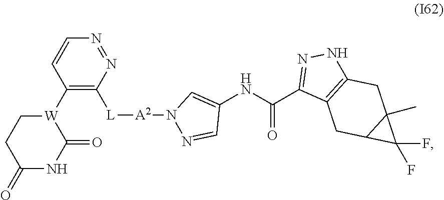

wherein the variables are as described below.

In certain embodiments, provided herein are compounds of Formula (I62A), or a stereoisomer and/or a pharmaceutically acceptable salt thereof:

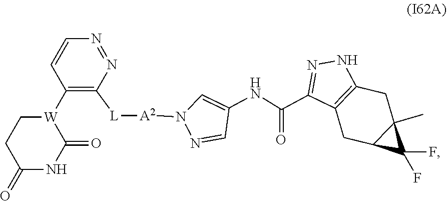

wherein the variables are as described below.

In certain embodiments, provided herein are compounds of Formula (I63), or a stereoisomer and/or a pharmaceutically acceptable salt thereof:

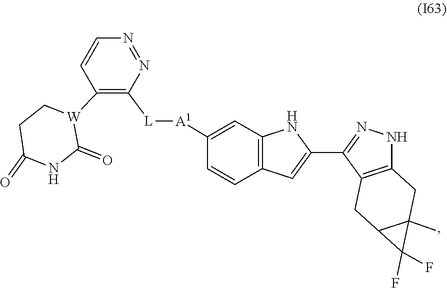

wherein the variables are as described below.

In certain embodiments, provided herein are compounds of Formula (I63A), or a stereoisomer and/or a pharmaceutically acceptable salt thereof:

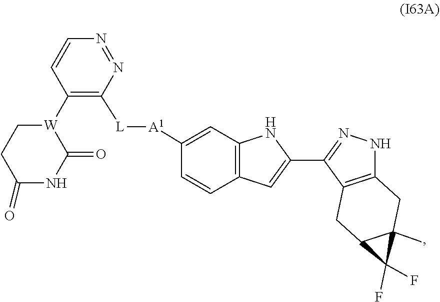

wherein the variables are as described below.

In certain embodiments, provided herein are compounds of Formula (I64), or a stereoisomer and/or a pharmaceutically acceptable salt thereof:

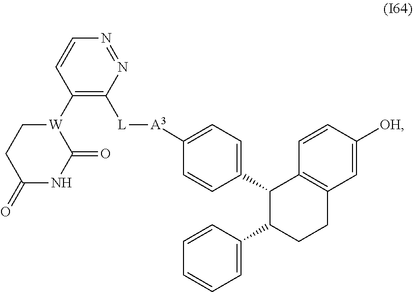

wherein the variables are as described below.

In certain embodiments, provided herein are compounds of Formula (I65), or a stereoisomer and/or a pharmaceutically acceptable salt thereof:

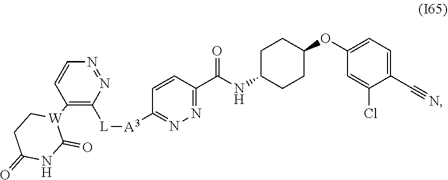

wherein the variables are as described below.

In certain embodiments, provided herein are compounds of Formula (I66), or a stereoisomer and/or a pharmaceutically acceptable salt thereof:

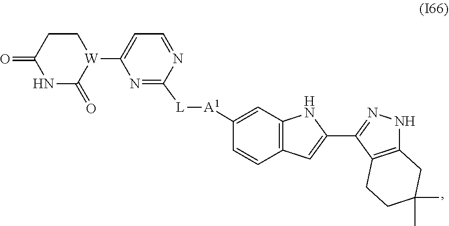

wherein the variables are as described below.

In certain embodiments, provided herein are compounds of Formula (I67), or a stereoisomer and/or a pharmaceutically acceptable salt thereof:

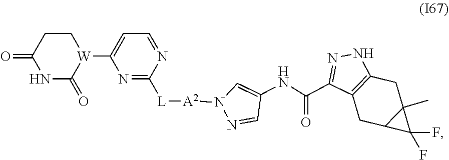

wherein the variables are as described below.

In certain embodiments, provided herein are compounds of Formula (I67A), or a stereoisomer and/or a pharmaceutically acceptable salt thereof:

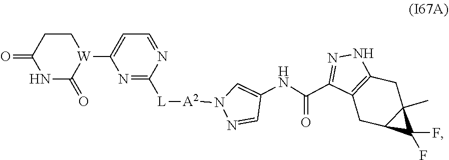

wherein the variables are as described below.

In certain embodiments, provided herein are compounds of Formula (I68), or a stereoisomer and/or a pharmaceutically acceptable salt thereof:

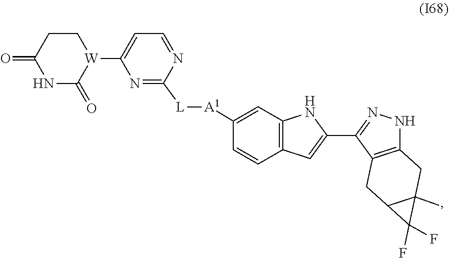

wherein the variables are as described below.

In certain embodiments, provided herein are compounds of Formula (I68A), or a stereoisomer and/or a pharmaceutically acceptable salt thereof:

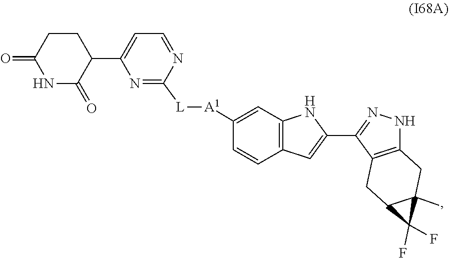

wherein the variables are as described below.

In certain embodiments, provided herein are compounds of Formula (I69), or a stereoisomer and/or a pharmaceutically acceptable salt thereof:

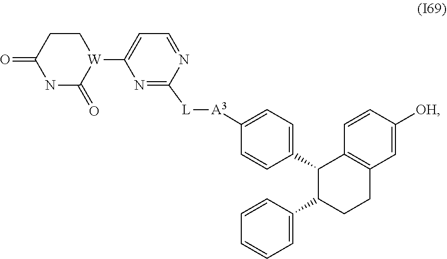

wherein the variables are as described below.

In certain embodiments, provided herein are compounds of Formula (I70), or a stereoisomer and/or a pharmaceutically acceptable salt thereof:

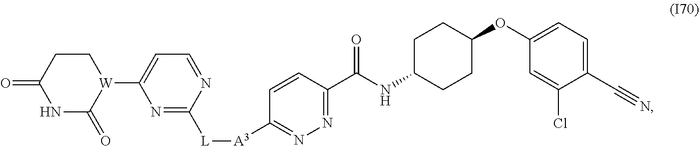

wherein the variables are as described below.

In certain embodiments, provided herein are compounds of Formula (I71), or a stereoisomer and/or a pharmaceutically acceptable salt thereof:

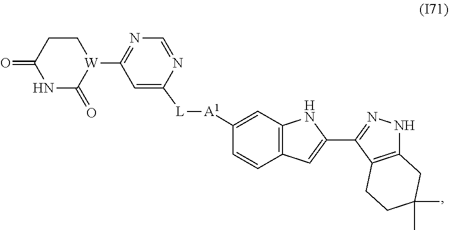

wherein the variables are as described below.

In certain embodiments, provided herein are compounds of Formula (I72), or a stereoisomer and/or a pharmaceutically acceptable salt thereof:

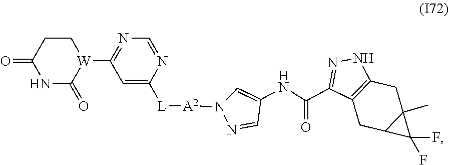

wherein the variables are as described below.

In certain embodiments, provided herein are compounds of Formula (I72A), or a stereoisomer and/or a pharmaceutically acceptable salt thereof:

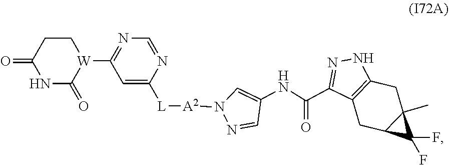

wherein the variables are as described below.

In certain embodiments, provided herein are compounds of Formula (I73), or a stereoisomer and/or a pharmaceutically acceptable salt thereof:

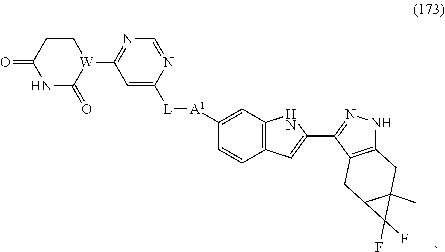

wherein the variables are as described below.

In certain embodiments, provided herein are compounds of Formula (I73A), or a stereoisomer and/or a pharmaceutically acceptable salt thereof:

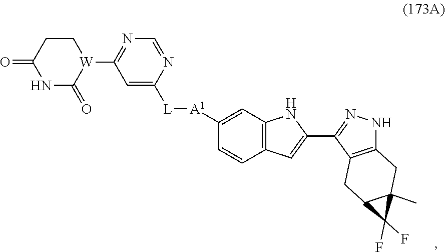

wherein the variables are as described below.

In certain embodiments, provided herein are compounds of Formula (I74), or a stereoisomer and/or a pharmaceutically acceptable salt thereof:

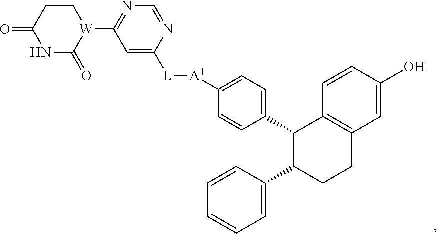

wherein the variables are as described below.

In certain embodiments, provided herein are compounds of Formula (I75), or a stereoisomer and/or a pharmaceutically acceptable salt thereof:

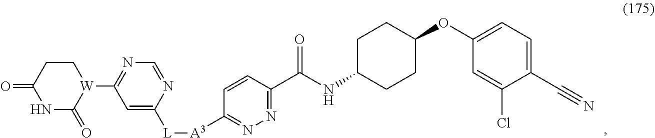

wherein the variables are as described below.

In certain embodiments, provided herein are compounds of Formula (I76), or a stereoisomer and/or a pharmaceutically acceptable salt thereof:

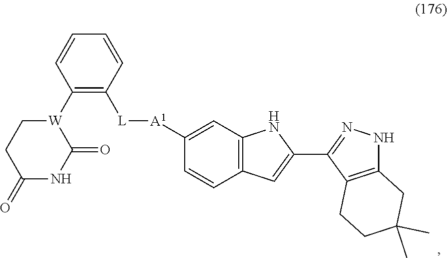

wherein the variables are as described below.

In certain embodiments, provided herein are compounds of Formula (I77), or a stereoisomer and/or a pharmaceutically acceptable salt thereof:

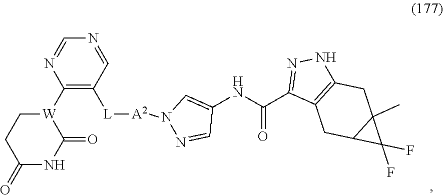

wherein the variables are as described below.

In certain embodiments, provided herein are compounds of Formula (I77A), or a stereoisomer and/or a pharmaceutically acceptable salt thereof:

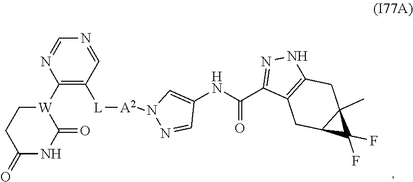

wherein the variables are as described below.

In certain embodiments, provided herein are compounds of Formula (I78), or a stereoisomer and/or a pharmaceutically acceptable salt thereof:

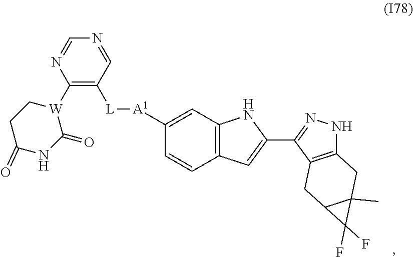

wherein the variables are as described below.

In certain embodiments, provided herein are compounds of Formula (I78A), or a stereoisomer and/or a pharmaceutically acceptable salt thereof:

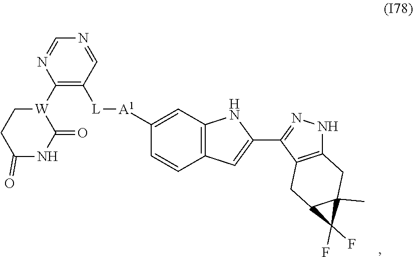

wherein the variables are as described below.

In certain embodiments, provided herein are compounds of Formula (I79), or a stereoisomer and/or a pharmaceutically acceptable salt thereof:

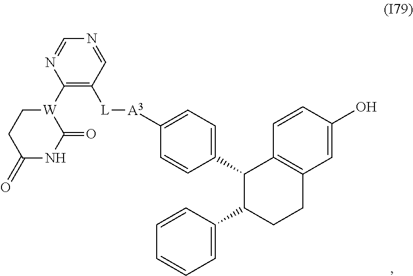

wherein the variables are as described below.

In certain embodiments, provided herein are compounds of Formula (I80), or a stereoisomer and/or a pharmaceutically acceptable salt thereof:

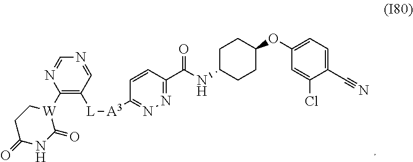

wherein the variables are as described below.

In certain embodiments, provided herein are compounds of Formula (I81), or a stereoisomer and/or a pharmaceutically acceptable salt thereof:

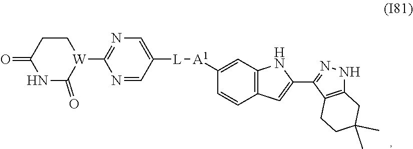

wherein the variables are as described below.

In certain embodiments, provided herein are compounds of Formula (I82), or a stereoisomer and/or a pharmaceutically acceptable salt thereof:

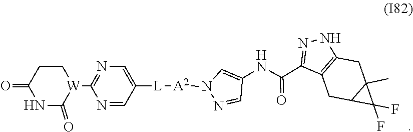

wherein the variables are as described below.

In certain embodiments, provided herein are compounds of Formula (I82A), or a stereoisomer and/or a pharmaceutically acceptable salt thereof:

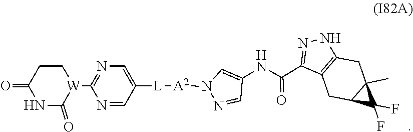

wherein the variables are as described below.

In certain embodiments, provided herein are compounds of Formula (I83), or a stereoisomer and/or a pharmaceutically acceptable salt thereof:

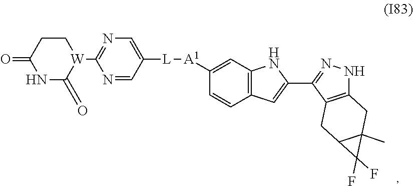

wherein the variables are as described below.

In certain embodiments, provided herein are compounds of Formula (I83A), or a stereoisomer and/or a pharmaceutically acceptable salt thereof:

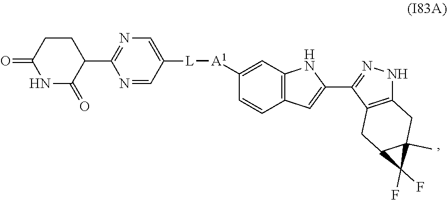

wherein the variables are as described below.

In certain embodiments, provided herein are compounds of Formula (I84), or a stereoisomer and/or a pharmaceutically acceptable salt thereof:

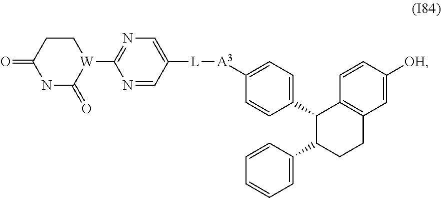

wherein the variables are as described below.

In certain embodiments, provided herein are compounds of Formula (I85), or a stereoisomer and/or a pharmaceutically acceptable salt thereof:

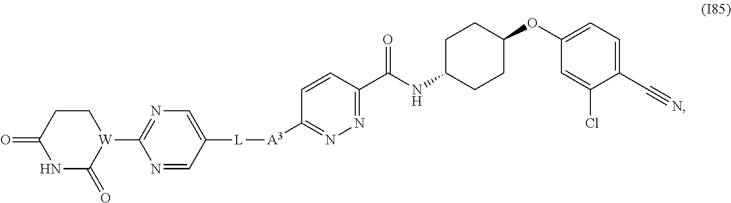

wherein the variables are as described below.

In certain embodiments, provided herein are compounds of Formula (I86), or a stereoisomer and/or a pharmaceutically acceptable salt thereof:

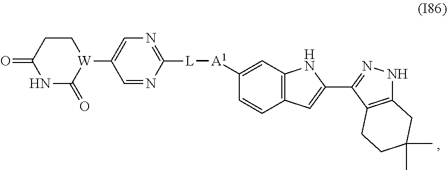

wherein the variables are as described below.

In certain embodiments, provided herein are compounds of Formula (I87), or a stereoisomer and/or a pharmaceutically acceptable salt thereof:

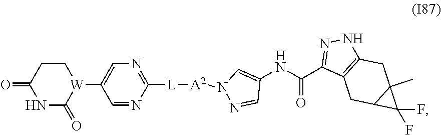

wherein the variables are as described below.

In certain embodiments, provided herein are compounds of Formula (I87A), or a stereoisomer and/or a pharmaceutically acceptable salt thereof:

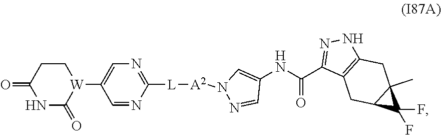

wherein the variables are as described below.

In certain embodiments, provided herein are compounds of Formula (I88), or a stereoisomer and/or a pharmaceutically acceptable salt thereof:

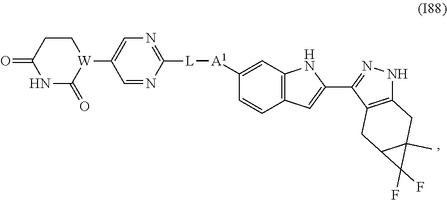

wherein the variables are as described below.

In certain embodiments, provided herein are compounds of Formula (I88A), or a stereoisomer and/or a pharmaceutically acceptable salt thereof:

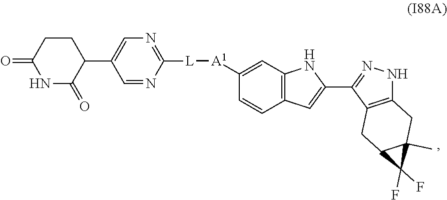

wherein the variables are as described below.

In certain embodiments, provided herein are compounds of Formula (I89), or a stereoisomer and/or a pharmaceutically acceptable salt thereof:

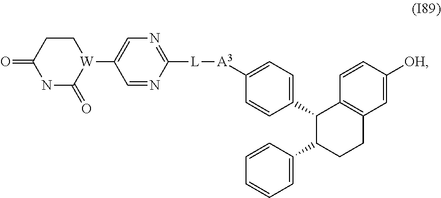

wherein the variables are as described below.

In certain embodiments, provided herein are compounds of Formula (I90), or a stereoisomer and/or a pharmaceutically acceptable salt thereof:

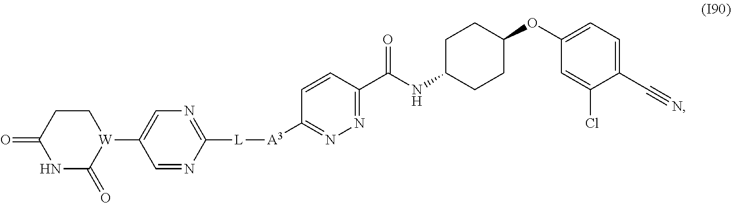

wherein the variables are as described below.

In certain embodiments, provided herein are compounds of Formula (I91), or a stereoisomer and/or a pharmaceutically acceptable salt thereof:

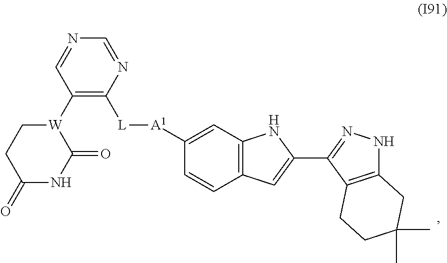

wherein the variables are as described below.

In certain embodiments, provided herein are compounds of Formula (I92), or a stereoisomer and/or a pharmaceutically acceptable salt thereof:

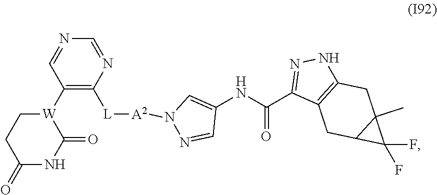

wherein the variables are as described below.

In certain embodiments, provided herein are compounds of Formula (I92A), or a stereoisomer and/or a pharmaceutically acceptable salt thereof:

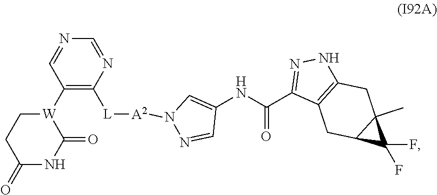

wherein the variables are as described below.

In certain embodiments, provided herein are compounds of Formula (I93), or a stereoisomer and/or a pharmaceutically acceptable salt thereof:

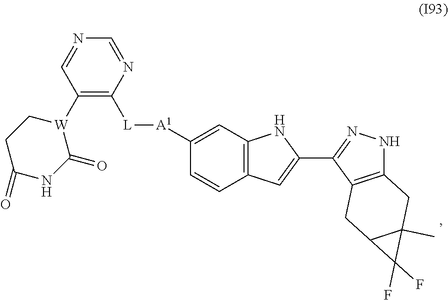

wherein the variables are as described below.

In certain embodiments, provided herein are compounds of Formula (I93A), or a stereoisomer and/or a pharmaceutically acceptable salt thereof:

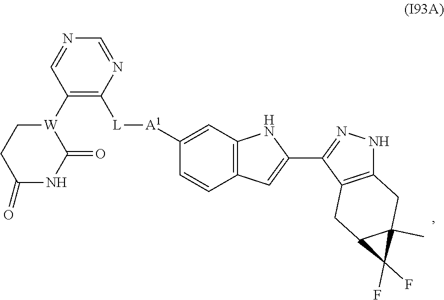

wherein the variables are as described below.

In certain embodiments, provided herein are compounds of Formula (I94), or a stereoisomer and/or a pharmaceutically acceptable salt thereof:

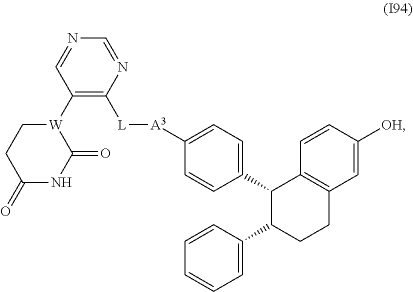

wherein the variables are as described below.

In certain embodiments, provided herein are compounds of Formula (I95), or a stereoisomer and/or a pharmaceutically acceptable salt thereof:

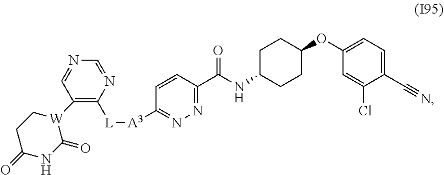

wherein the variables are as described below.

In certain embodiments, provided herein are compounds of Formula (I96), or a stereoisomer and/or a pharmaceutically acceptable salt thereof:

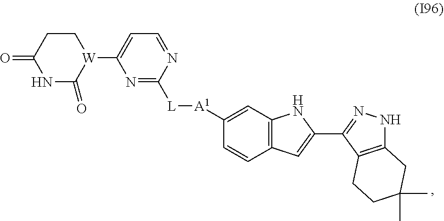

wherein the variables are as described below.

In certain embodiments, provided herein are compounds of Formula (I97), or a stereoisomer and/or a pharmaceutically acceptable salt thereof:

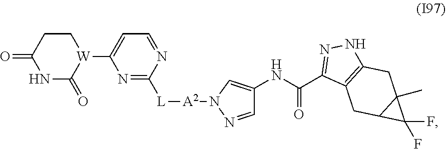

wherein the variables are as described below.

In certain embodiments, provided herein are compounds of Formula (I97A), or a stereoisomer and/or a pharmaceutically acceptable salt thereof:

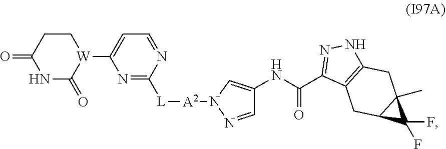

wherein the variables are as described below.

In certain embodiments, provided herein are compounds of Formula (I98), or a stereoisomer and/or a pharmaceutically acceptable salt thereof:

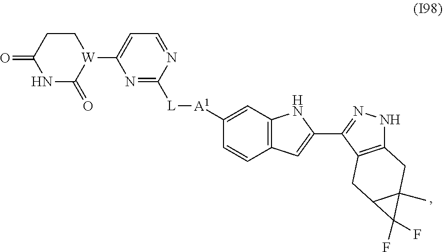

wherein the variables are as described below.

In certain embodiments, provided herein are compounds of Formula (I98A), or a stereoisomer and/or a pharmaceutically acceptable salt thereof:

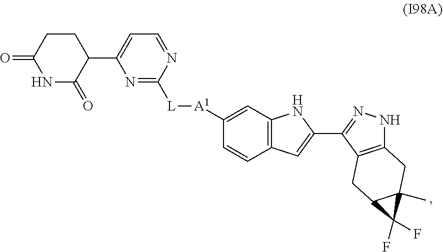

wherein the variables are as described below.

In certain embodiments, provided herein are compounds of Formula (I99), or a stereoisomer and/or a pharmaceutically acceptable salt thereof:

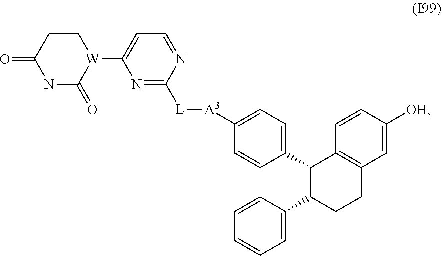

wherein the variables are as described below.

In certain embodiments, provided herein are compounds of Formula (I100), or a stereoisomer and/or a pharmaceutically acceptable salt thereof:

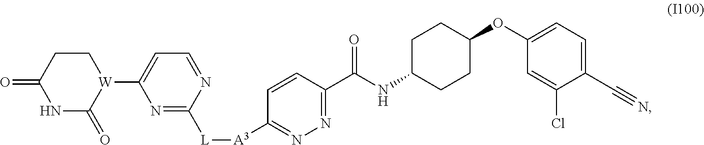

wherein the variables are as described below.

In certain embodiments, provided herein are compounds of Formula (I101), or a stereoisomer and/or a pharmaceutically acceptable salt thereof:

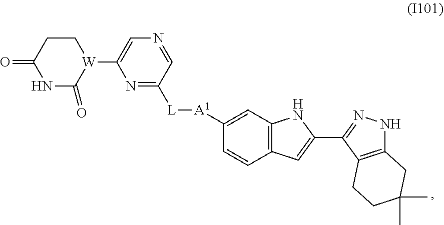

wherein the variables are as described below.

In certain embodiments, provided herein are compounds of Formula (I102), or a stereoisomer and/or a pharmaceutically acceptable salt thereof:

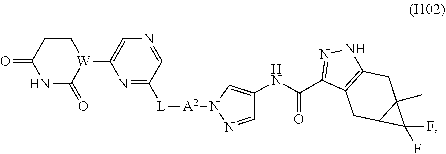

wherein the variables are as described below.

In certain embodiments, provided herein are compounds of Formula (I102A), or a stereoisomer and/or a pharmaceutically acceptable salt thereof:

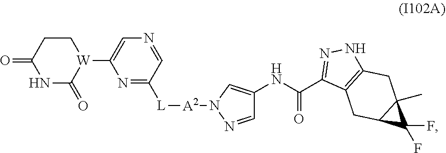

wherein the variables are as described below.

In certain embodiments, provided herein are compounds of Formula (I103), or a stereoisomer and/or a pharmaceutically acceptable salt thereof:

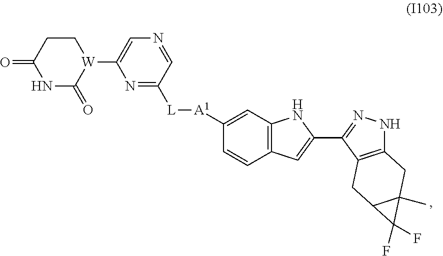

wherein the variables are as described below.

In certain embodiments, provided herein are compounds of Formula (I102A), or a stereoisomer and/or a pharmaceutically acceptable salt thereof:

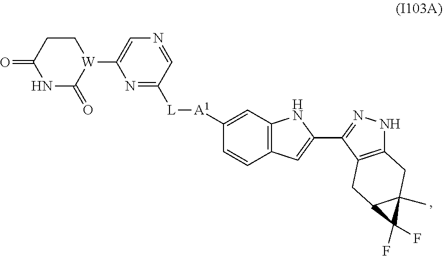

wherein the variables are as described below.

In certain embodiments, provided herein are compounds of Formula (I104), or a stereoisomer and/or a pharmaceutically acceptable salt thereof:

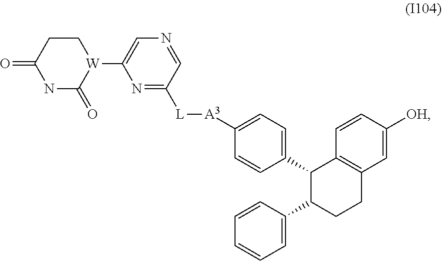

wherein the variables are as described below.

In certain embodiments, provided herein are compounds of Formula (I105), or a stereoisomer and/or a pharmaceutically acceptable salt thereof:

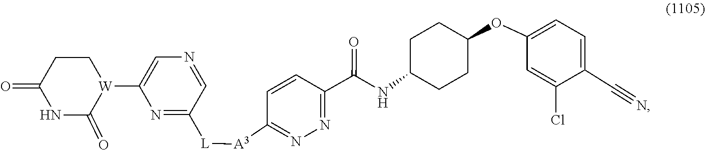

wherein the variables are as described below.

In certain embodiments, provided herein are compounds of Formula (I106), or a stereoisomer and/or a pharmaceutically acceptable salt thereof:

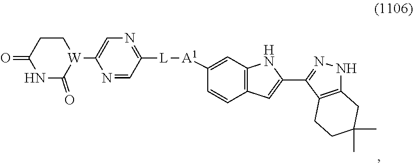

wherein the variables are as described below.

In certain embodiments, provided herein are compounds of Formula (I107), or a stereoisomer and/or a pharmaceutically acceptable salt thereof:

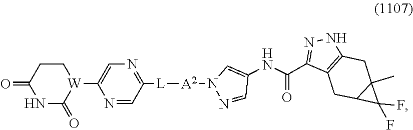

wherein the variables are as described below.

In certain embodiments, provided herein are compounds of Formula (I107A), or a stereoisomer and/or a pharmaceutically acceptable salt thereof:

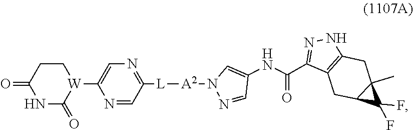

wherein the variables are as described below.

In certain embodiments, provided herein are compounds of Formula (I108), or a stereoisomer and/or a pharmaceutically acceptable salt thereof:

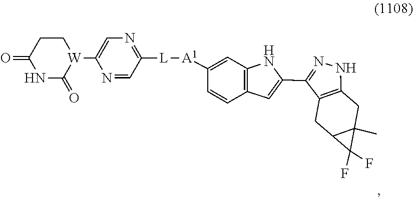

wherein the variables are as described below.

In certain embodiments, provided herein are compounds of Formula (I108A), or a stereoisomer and/or a pharmaceutically acceptable salt thereof:

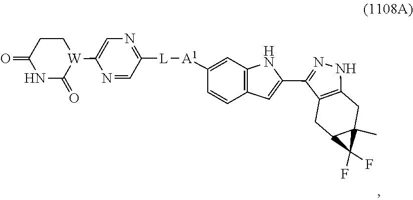

wherein the variables are as described below.

In certain embodiments, provided herein are compounds of Formula (I109), or a stereoisomer and/or a pharmaceutically acceptable salt thereof:

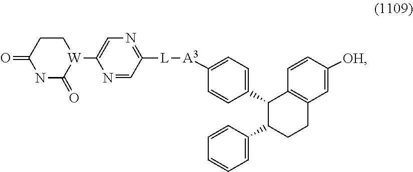

wherein the variables are as described below.

In certain embodiments, provided herein are compounds of Formula (I110), or a stereoisomer and/or a pharmaceutically acceptable salt thereof:

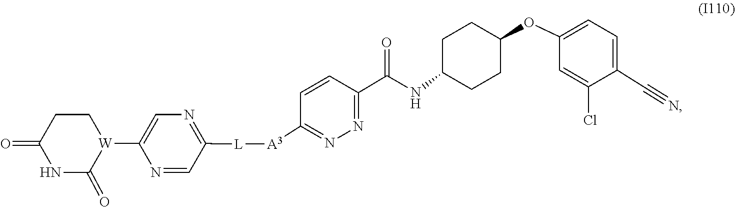

wherein the variables are as described below.

In certain embodiments, provided herein are compounds of Formula (I111), or a stereoisomer and/or a pharmaceutically acceptable salt thereof:

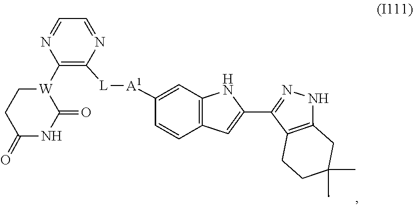

wherein the variables are as described below.

In certain embodiments, provided herein are compounds of Formula (I112), or a stereoisomer and/or a pharmaceutically acceptable salt thereof:

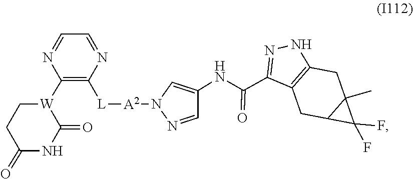

wherein the variables are as described below.

In certain embodiments, provided herein are compounds of Formula (I112A), or a stereoisomer and/or a pharmaceutically acceptable salt thereof:

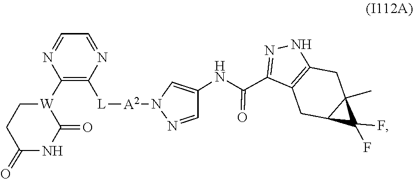

wherein the variables are as described below.

In certain embodiments, provided herein are compounds of Formula (I113), or a stereoisomer and/or a pharmaceutically acceptable salt thereof:

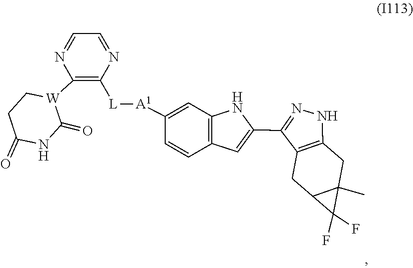

wherein the variables are as described below.

In certain embodiments, provided herein are compounds of Formula (I113A), or a stereoisomer and/or a pharmaceutically acceptable salt thereof:

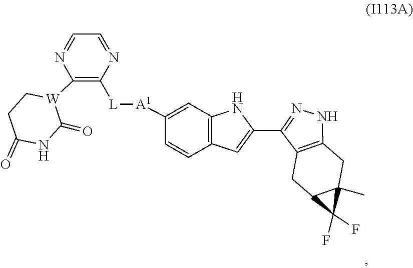

wherein the variables are as described below.

In certain embodiments, provided herein are compounds of Formula (I114), or a stereoisomer and/or a pharmaceutically acceptable salt thereof:

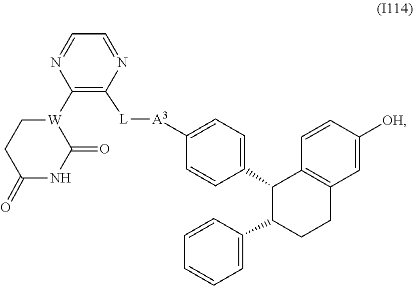

wherein the variables are as described below.

In certain embodiments, provided herein are compounds of Formula (I115), or a stereoisomer and/or a pharmaceutically acceptable salt thereof:

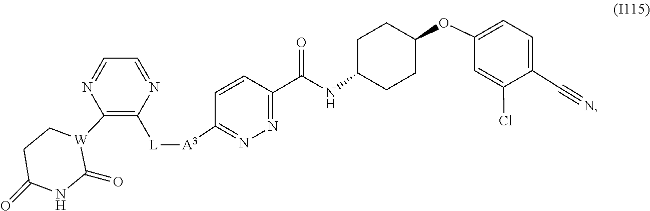

wherein the variables are as described below.

In certain embodiments, of any compound of Formula (I-XXIX), (Ia-If), or (I1-I115), L is a linker according to the formula -L¹-L²-L³-L⁴-L⁵-L⁶-L⁷-. Each group L^{x} is described in detail below. In certain embodiments, the linker comprises at least one heterocyclic group. In certain embodiments, the linker comprises at one heterocyclic group. In certain embodiments, the linker comprises two heterocyclic groups. In certain embodiments, the linker comprises three heterocyclic groups. In certain embodiments, the linker comprises at least one spiro bicyclic heterocycloalkylene groups. In certain embodiments, the linker comprises at one spiro bicyclic heterocycloalkylene group. In certain embodiments, the linker comprises at two spiro bicyclic heterocycloalkylene groups. In certain embodiments, the linker comprises at three spiro bicyclic heterocycloalkylene groups. In certain embodiments, the linker comprises at least one heterocycloalkylene group and at least one spiro bicyclic heterocycloalkylene. The remaining groups of the linker are selected for chemical compatibility with adjacent groups, as will be recognized by those of skill in the art.

In certain embodiments, L is a linker according to the formula -L¹-L²-L³-L⁴-L⁵-L⁶-L⁷-. In certain embodiments, is absent, —N(R²¹)—; C(R²²)—; C₁₋₈alkylene; C₂₋₈alkynylene; Q¹; or Q². In certain embodiments, each -L²-, -L³-, -L⁴-, and -L⁵- is independently, absent; —N(R²¹)—; C(R²²)—; —C(O)—; —O—; —(CH₂—CH₂—O)₁₋₈—; C₁₋₈alkylene; C₂₋₈alkynylene; Q¹; Q²; or Q³. In certain embodiments, each -L⁶- and -L⁷-, is independently, absent; —N(R²¹)—; —C(R²²)—; —C(O)—; —C(O)—N(R²¹)—; —N(R²¹)—C(O); or —C(R²²)—C(O)—N(R²¹). In certain embodiments, L comprises at least one Q¹. In certain embodiments, L comprises one Q¹. In certain embodiments, L comprises two Q¹. In certain embodiments, L comprises three Q¹. In certain embodiments, L comprises at least one Q². In certain embodiments, L comprises one Q². In certain embodiments, L comprises two Q². In certain embodiments, L comprises three Q². In certain embodiments, L comprises at least one Q³ and at least one Q². In certain embodiments, L comprises one Q³ and one Q².

In certain embodiments, each Q¹ is a three- to seven-membered heterocycloalkylene comprising at least one nitrogen. In certain embodiments, each Q² is a five- to thirteen-membered spiro bicyclic heterocycloalkylene comprising at least one nitrogen. In certain embodiments, each Q³ is a three- to six-membered cycloalkylene. In certain embodiments, each R²¹ is hydrogen, or methyl. In certain embodiments, each R²² is hydrogen, methyl, aryl, or heteroaryl.

In certain embodiments, L comprises at least one -Q¹- according to

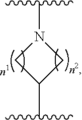

wherein n¹ is one, or two, and n² is one, or two.

In certain embodiments, L comprises at least one -Q¹- selected from the group consisting of

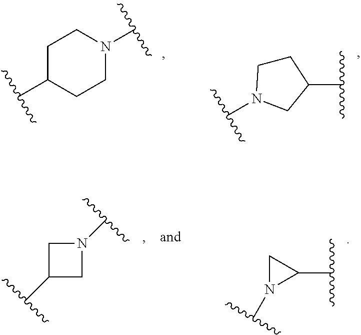 [SMILES: *C1CCN(*)CC1 ; *C1CCN(*)C1 ; *C1CN(*)C1 ; *C1CN1*]

In certain embodiments, L comprises at least one -Q²- according to

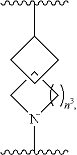

wherein n³ is one or two.

In certain embodiments, L comprises at least one -Q²- according to

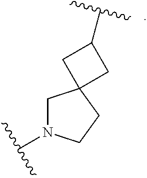 [SMILES: *C1CC2(CCN(*)C2)C1]

In certain embodiments, L comprises at least one -Q²- according to

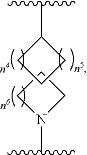

wherein n⁴ is one, or two, n⁵ is one, or two, and n⁶ is one, or two.

In certain embodiments, L comprises at least one -Q²- according to

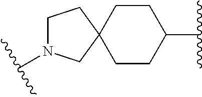 [SMILES: *C1CCC2(CC1)CCN(*)C2]

In certain embodiments, L comprises at least one -Q²- according to

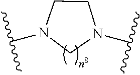

wherein n⁸ is one, or two.

In certain embodiments, L comprises at least one -Q²- according to

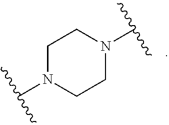 [SMILES: *N1CCN(*)CC1]

In certain embodiments, L comprises at least one -Q²- according to

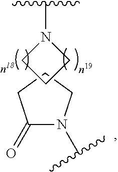

wherein n¹⁸ and n¹⁹ is two, or piperidinyl; or when n¹⁸ is two, then n¹⁹ is three, or azepanyl; or when n¹⁸ is three, then n¹⁹ is two, or azepanyl.

In certain embodiments, L comprises at least one -Q²- according to

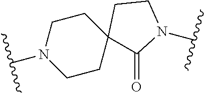 [SMILES: *N1CCC2(CC1)CCN(*)C2=O]

In certain embodiments, L comprises at least one -Q²- according to

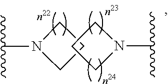

wherein n²² is zero to two; n²³ is zero to two, and n²⁴ is one, or two. In certain embodiments, when n²² is two, then n²³ and n²⁴ is one; or when n²² is two, then n²³ and n²⁴ is two.

In certain embodiments, L comprises at least one -Q²- according to

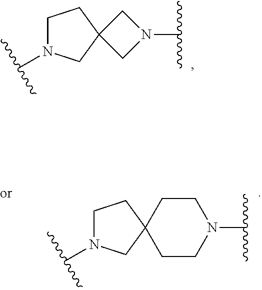 [SMILES: *N1CCC2(C1)CN(*)C2 ; *N1CCC2(CC1)CCN(*)C2]

In certain embodiments, L comprises at least one -Q²- according to

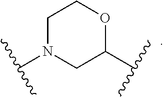 [SMILES: *C1CN(*)CCO1]

In certain embodiments, L comprises at least one -Q³- according to

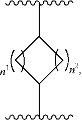

wherein n¹ is one, or two, and n² is one, or two.

In certain embodiments, L comprises at least one -Q³- selected from the group consisting of

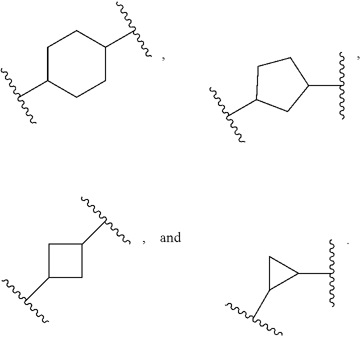 [SMILES: *C1CCC(*)CC1 ; *C1CCC(*)C1 ; *C1CC(*)C1 ; *C1CC1*]

In certain embodiments, of any compound of Formula (I-XXIX), (Ia-If), or (I1-I115), L is selected from:

-Q¹-N(Me)-CH₂-Q¹-C(O)—;
—N(Me)-Q¹-CH₂-Q¹-C(O)—;
-Q²-CH₂-Q¹-C(O)—;
-Q¹-CH₂-Q¹-C(O)—;
-Q¹-Q¹-C(O)—;
-Q¹-CH₂—N(Me)-Q¹-C(O)—;
-Q¹-CH₂-Q¹-CH₂—C(O)—N(Me)-;
-Q¹-N(Me)-CH₂-Q¹-C(O)—;
-Q¹-CH₂-Q²-;
-Q¹-CH₂-Q¹-N(H)—;
-Q¹-CH₂-Q²-N(H)—;
-Q¹-CH₂-Q¹-CH₂-Q₁-;
-Q³-CH₂—CH₂-Q¹-;
-Q³-CH₂—CH₂-Q²-;
-Q¹-C(O)-Q¹-;
-Q¹-C(O)-Q²-;
-Q¹-CH₂-Q¹-N(Me)-C(O)—;
—CH₂—CH₂—CH₂—CH₂-Q¹-C(O)—;
-Q¹-CH₂-Q¹-C(O)—;

### -Q¹-C(O)—;

-Q¹-C(O)-Q¹-C(C₆H₅)—;
—C≡CCH₂-Q¹-C(O)—;
-Q¹-C(O)-Q²-;
-Q¹-CH₂—CH₂-Q²-;
-Q¹-CH₂-Q¹-N—C(O)—;
—CH₂—CH₂—CH₂-Q¹-C(O)—;
-Q¹-CH₂-Q¹-C(Me)-C(O)—N(Me)-;
Q¹-Q¹-C(O)—;
—CH₂-Q¹-;
-Q¹-C(O)-Q¹-CH₂—;
—N(H)—(CH₂)₅—C(O)-Q¹-C(C₆H₅)—;
—N(H)—(CH₂)₂—O—(CH₂)₂—C(O)-Q¹-C(C₆H₅)—;
-Q¹-(CH₂)₃—C(O)-Q¹-C(C₆H₅)—;
-Q²-C(O)-Q¹-C(C₆H₅)—;
-Q²-CH₂—C(O)-Q¹-C(C₆H₅)—;
-Q²-(CH₂)₃—C(O)-Q′-C(C₆H₅)—;
-Q²-(CH₂)₂—C(O)-Q′-C(C₆H₅)—;
—(CH₂)₆-Q¹-C(C₆H₅)—;
-Q¹-Q¹-C(O)-Q¹-C(C₆H₅)—;
-Q¹-CH₂—C(O)-Q¹-C(C₆H₅)—;
-Q¹-(CH₂)₂—C(O)-Q¹-C(C₆H₅)—;
-Q¹-(CH₂)₃—C(O)-Q¹-C(C₆H₅)—;
—(CH₂)₃—C(O)-Q¹-C(C₆H₅)—;
—(CH₂)₄—C(O)-Q¹-C(C₆H₅)—;
—(CH₂)₅—C(O)-Q¹-C(C₆H₅)—;
—(CH₂)₆—C(O)-Q¹-C(C₆H₅)—;
—(CH₂)₃-Q¹-CH₂—C(O)-Q¹-C(C₆H₅)—;
—(CH₂)₆-Q¹-C(C₆H₅)—;
—(CH₂)₆-Q¹-C(thiazolyl)-;
—(CH₂)₃—O-Q³-C(O)-Q¹-C(C₆H₅)—;
—(CH₂)₃—O—(CH₂)₂—C(O)-Q¹-C(C₆H₅)—;
—(CH₂)₃—O—(CH₂)₂—C(O)-Q¹-C(thiazolyl)-;
—(CH₂)₃—O—(CH₂)₂—C(O)-Q¹-C(pyrid-2-yl)-;
—(CH₂)₄-Q¹-C(C₆H₅)—;
—(CH₂)₅-Q¹-C(C₆H₅)—;
—(CH₂)₆-Q¹-C(C₆H₅)—;
—(CH₂)₆-Q¹-C(thiazolyl)-;
—(CH₂)₆-Q¹-C(pyrid-2-yl)-;
—(CH₂)₇-Q¹-C(C₆H₅)—;
—(CH₂)₇-Q¹-C(Me)-C(O)—N(Me)-;
—N(H)—(CH₂)₂—O—(CH₂)₂-Q¹-C(Me)-C(O)—N(Me)-;
—(CH₂)₃—O—(CH₂)₂—C(O)-Q¹-C(Me)-C(O)—N(Me)-;
—N(H)—(CH₂)₂—O—(CH₂)₂-Q¹-C(C₆H₅)—;
—N(H)—(CH₂)₂—O—(CH₂)₂—C(O)-Q¹-C(C₆H₅)—;
—N(H)—(CH₂)₂—[O—(CH₂)₂]₂—C(O)-Q¹-C(C₆H₅)—;
—N(H)—(CH₂)₂—[O—(CH₂)₂]₃—C(O)-Q¹-C(C₆H₅)—;
—N(H)—(CH₂)₂—[O—(CH₂)₂]₄—C(O)-Q¹-C(C₆H₅)—;
—N(H)—(CH₂)₂—[O—(CH₂)₂]₅—C(O)-Q¹-C(C₆H₅)—;
—N(H)—(CH₂)₂—[O—(CH₂)₂]₆—C(O)-Q¹-C(C₆H₅)—;
—N(H)—(CH₂)₂—[O—(CH₂)₂]₇—C(O)-Q¹-C(C₆H₅)—;
—N(H)—(CH₂)₂—[O—(CH₂)₂]₈—C(O)-Q¹-C(C₆H₅)—;
—N(H)-Q³-O—(CH₂)₂—CH₂—;
—N(H)—(CH₂)₃-Q¹-(CH₂)₂—;
—C(O)—N(H)—[(CH₂)₃—O]₃—(CH₂)₂—NH—;
—C(O)—N(H)—[(CH₂)₃—O]₃—(CH₂)₂—;
-Q¹-C(O)—[(CH₂)₂—O]₃—(CH₂)₂—NH—;
-Q¹-(CH₂)₃—O—CH₂—;
-Q¹-C(O)—(C₆H₆)—CH₂—;
-Q¹-(2-pyridyl)-O—CH₂—;
—N(H)-Q³-X¹-(2-pyridyl)-O—;
—N(H)-Q³-X¹-(4-pyridyl)-;
—N(H)—(CH₂)₂-Q³-X¹-(2-pyridyl)-O—CH₂—;
—CH≡C—(CH₂)₂Q¹-; and
-Q¹-, wherein X¹ is oxygen, or sulfur.

In certain embodiments, of any compound of Formula (I-XXIX), (Ia-If), or (I1-I115), the linker group is selected from:

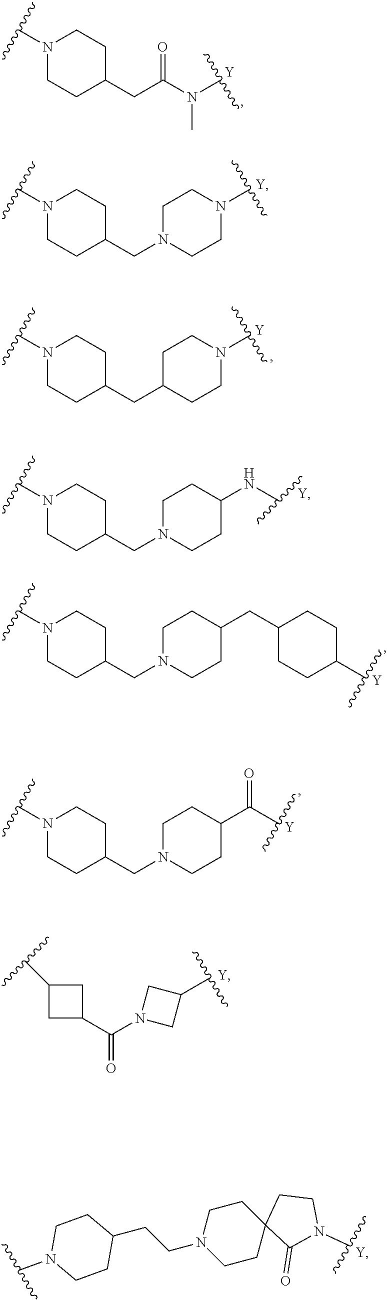

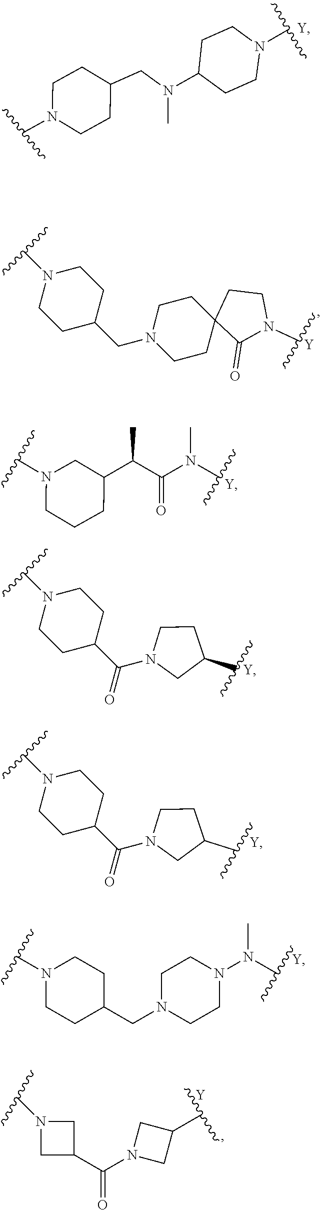

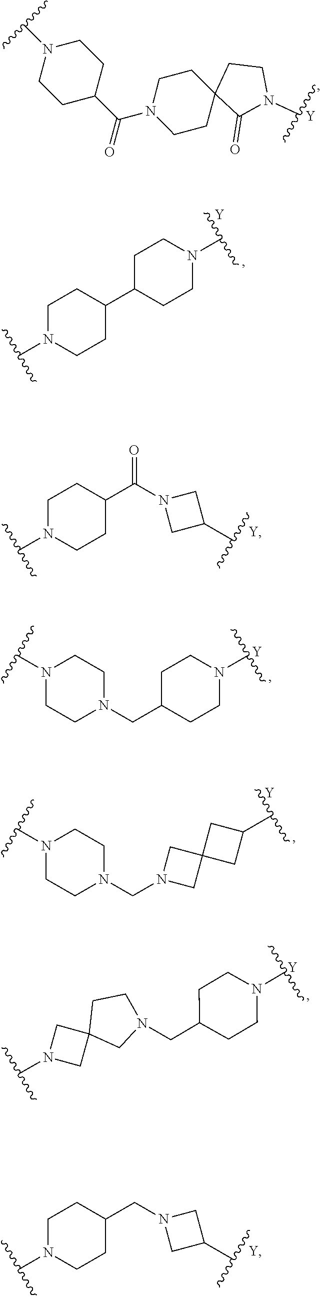

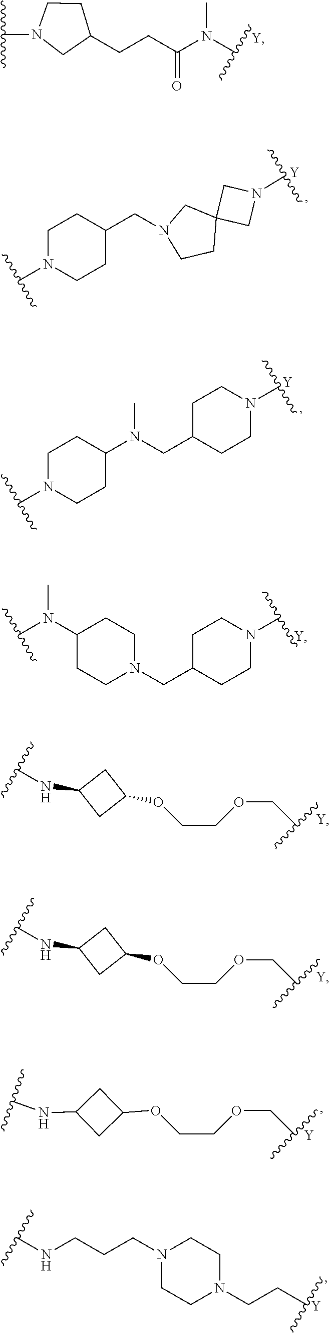

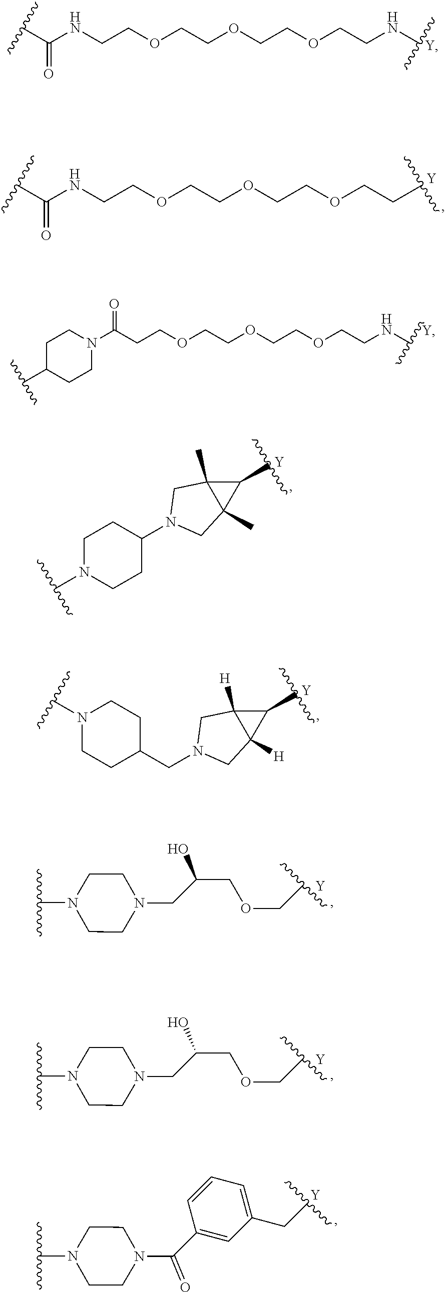

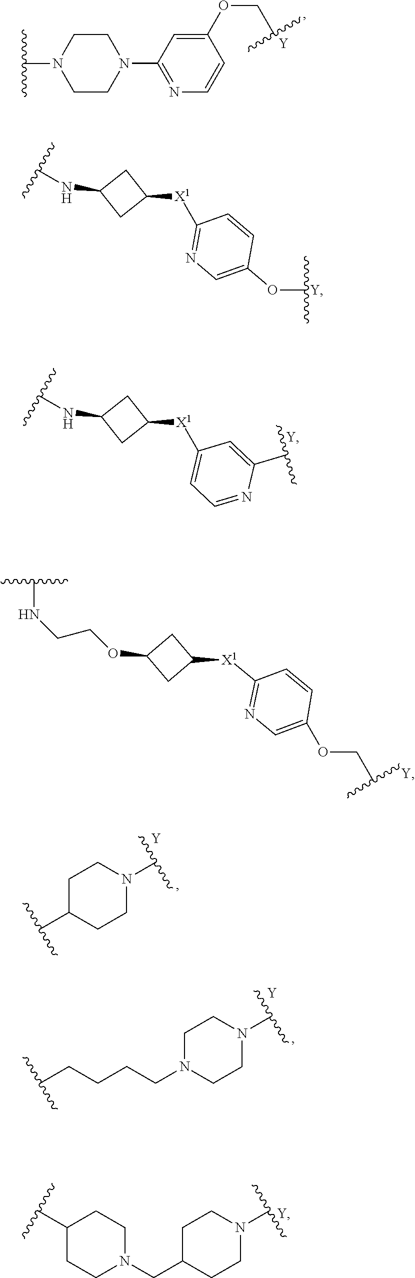

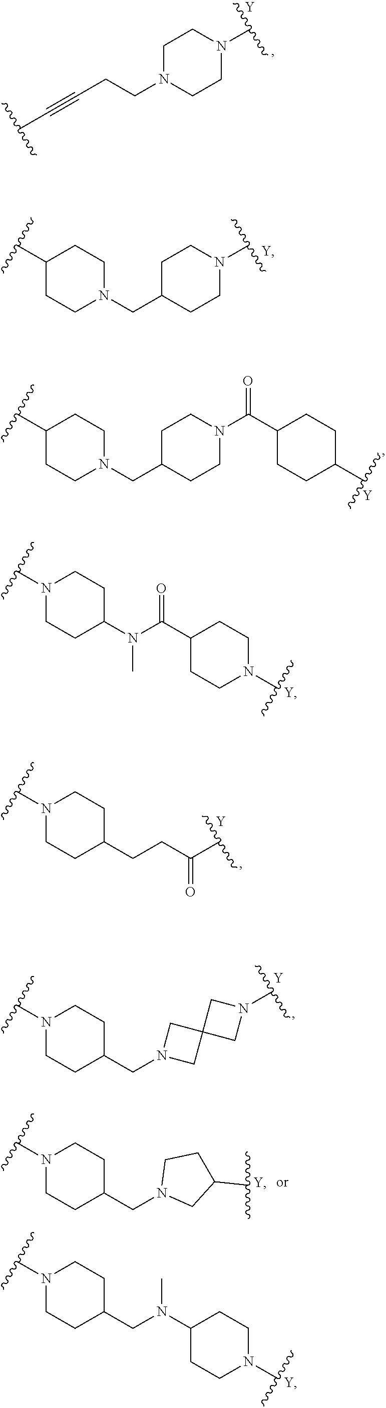

wherein, [symbol] designates attachment to Y.

In certain embodiments, provided herein are compounds of Table 1 below, or a pharmaceutically salt, stereoisomer, and/or mixture of stereoisomers thereof.

TABLE 1

| Cpd. | | |
|---|---|---|
| No. | Structure | Target |
| 1 | 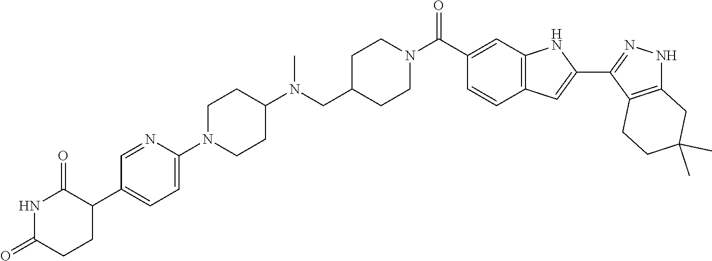 [SMILES: CN(CC1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1] | ITK |
| 2 | 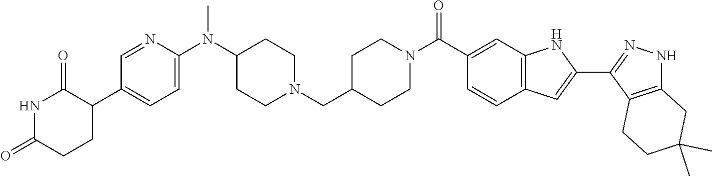 [SMILES: CN(c1ccc(C2CCC(=O)NC2=O)cn1)C1CCN(CC2CCN(C(=O)c3ccc4cc(-c5n[nH]c6c5CCC(C)(C)C6)[nH]c4c3)CC2)CC1] | ITK |
| 3 | 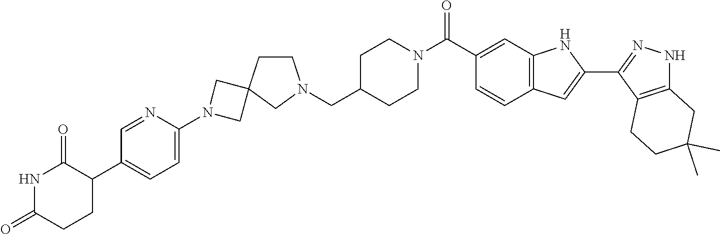 [SMILES: CC1(C)CCc2c(-c3cc4ccc(C(=O)N5CCC(CN6CCC7(C6)CN(c6ccc(C8CCC(=O)NC8=O)cn6)C7)CC5)cc4[nH]3)n[nH]c2C1] | ITK |
| 4 | 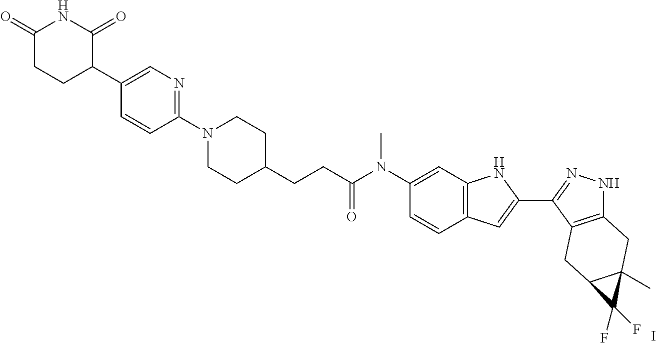 | ITK |
| 5 | 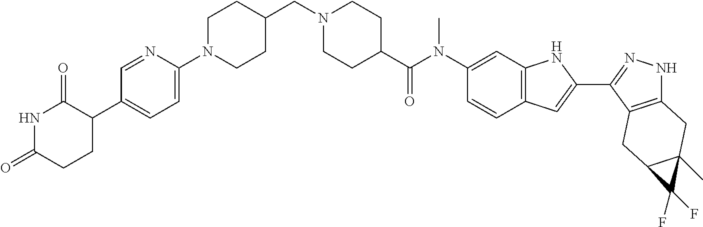 [SMILES: CN(C(=O)C1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1] | ITK |
| 6 | 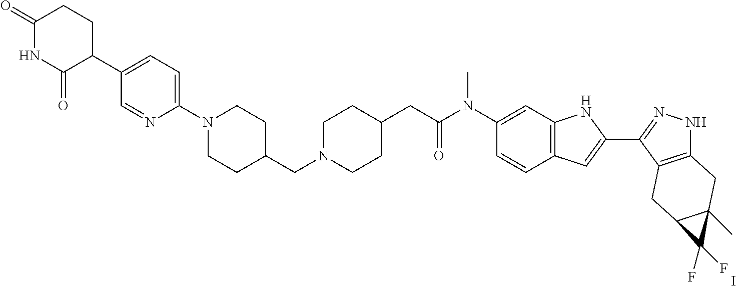 | ITK |
| 7 | 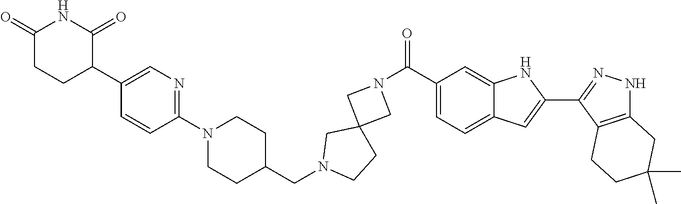 [SMILES: CC1(C)CCc2c(-c3cc4ccc(C(=O)N5CC6(CCN(CC7CCN(c8ccc(C9CCC(=O)NC9=O)cn8)CC7)C6)C5)cc4[nH]3)n[nH]c2C1] | ITK |
| 8 | 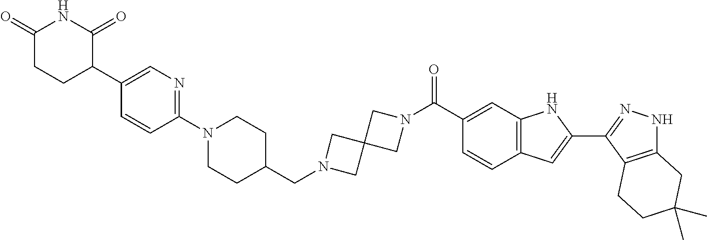 [SMILES: CC1(C)CCc2c(-c3cc4ccc(C(=O)N5CC6(CN(CC7CCN(c8ccc(C9CCC(=O)NC9=O)cn8)CC7)C6)C5)cc4[nH]3)n[nH]c2C1] | ITK |
| 9 | 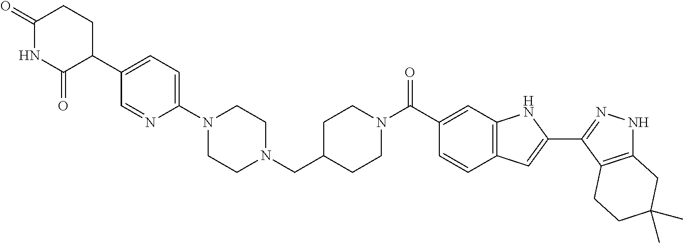 [SMILES: CC1(C)CCc2c(-c3cc4ccc(C(=O)N5CCC(CN6CCN(c7ccc(C8CCC(=O)NC8=O)cn7)CC6)CC5)cc4[nH]3)n[nH]c2C1] | ITK |
| 10 | 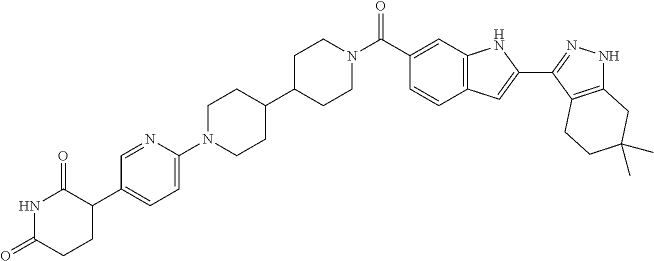 [SMILES: CC1(C)CCc2c(-c3cc4ccc(C(=O)N5CCC(C6CCN(c7ccc(C8CCC(=O)NC8=O)cn7)CC6)CC5)cc4[nH]3)n[nH]c2C1] | ITK |
| 11 | 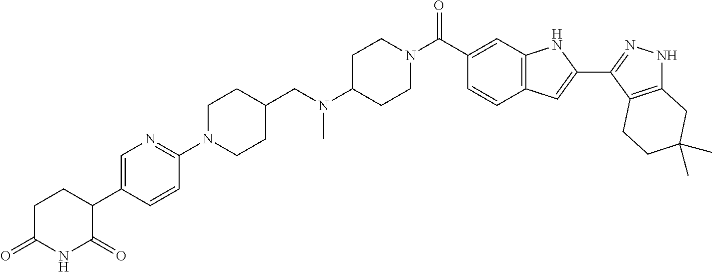 [SMILES: CN(CC1CCN(c2ccc(C3CCC(=O)NC3=O)cn2)CC1)C1CCN(C(=O)c2ccc3cc(-c4n[nH]c5c4CCC(C)(C)C5)[nH]c3c2)CC1] | ITK |
| 12 | 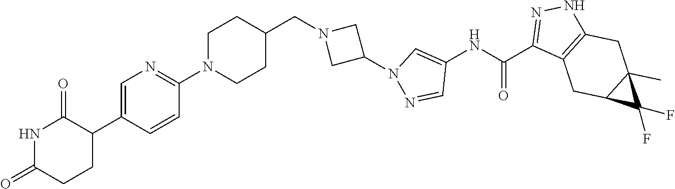 [SMILES: C[C@@]12Cc3[nH]nc(C(=O)Nc4cnn(C5CN(CC6CCN(c7ccc(C8CCC(=O)NC8=O)cn7)CC6)C5)c4)c3C[C@@H]1C2(F)F] | ITK |
| 13 | 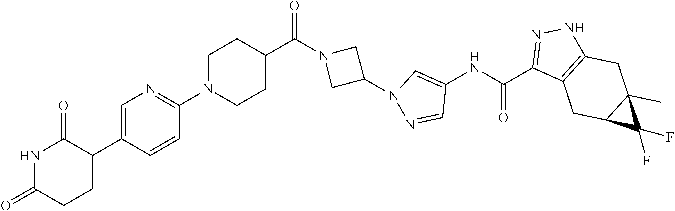 [SMILES: C[C@@]12Cc3[nH]nc(C(=O)Nc4cnn(C5CN(C(=O)C6CCN(c7ccc(C8CCC(=O)NC8=O)cn7)CC6)C5)c4)c3C[C@@H]1C2(F)F] | ITK |
| 14 | 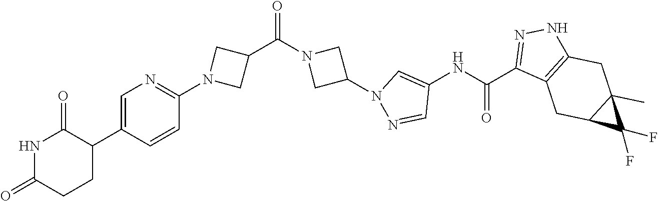 [SMILES: C[C@@]12Cc3[nH]nc(C(=O)Nc4cnn(C5CN(C(=O)C6CN(c7ccc(C8CCC(=O)NC8=O)cn7)C6)C5)c4)c3C[C@@H]1C2(F)F] | ITK |
| 15 | 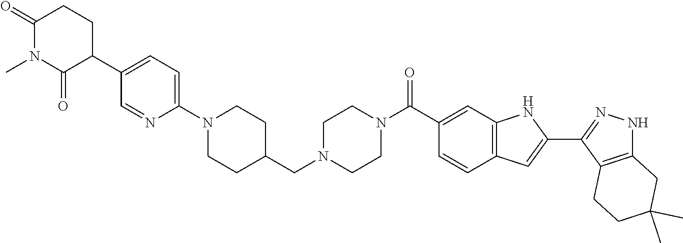 [SMILES: CN1C(=O)CCC(c2ccc(N3CCC(CN4CCN(C(=O)c5ccc6cc(-c7n[nH]c8c7CCC(C)(C)C8)[nH]c6c5)CC4)CC3)nc2)C1=O] | ITK |
| 16 | 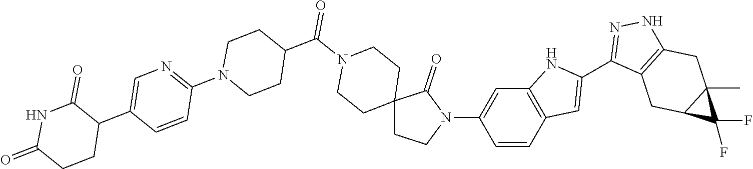 [SMILES: C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F] | ITK |
| 17 | 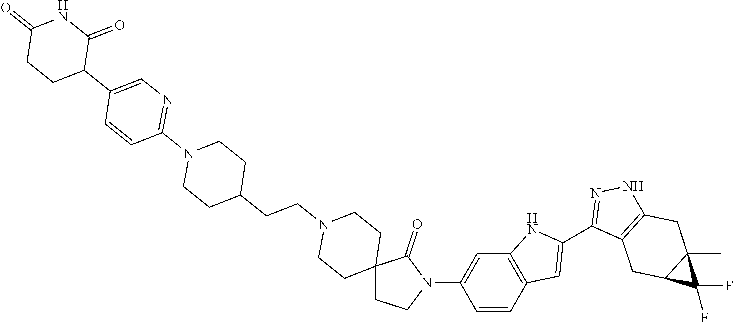 [SMILES: C[C@@]12Cc3[nH]nc(-c4cc5ccc(N6CCC7(CCN(CCC8CCN(c9ccc(C%10CCC(=O)NC%10=O)cn9)CC8)CC7)C6=O)cc5[nH]4)c3C[C@@H]1C2(F)F] | ITK |
| 18 | 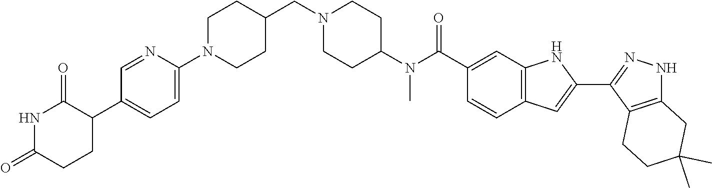 [SMILES: CN(C(=O)c1ccc2cc(-c3n[nH]c4c3CCC(C)(C)C4)[nH]c2c1)C1CCN(CC2CCN(c3ccc(C4CCC(=O)NC4=O)cn3)CC2)CC1] | ITK |
| 19 | 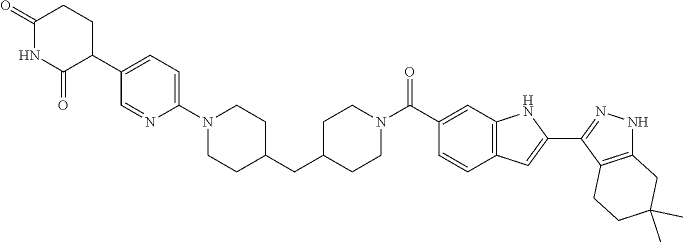 [SMILES: CC1(C)CCc2c(-c3cc4ccc(C(=O)N5CCC(CC6CCN(c7ccc(C8CCC(=O)NC8=O)cn7)CC6)CC5)cc4[nH]3)n[nH]c2C1] | ITK |
| 20 | 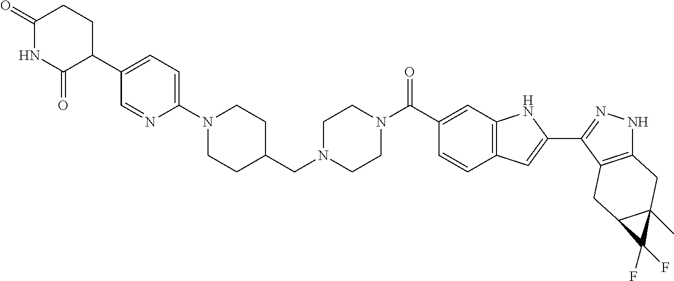 [SMILES: C[C@@]12Cc3[nH]nc(-c4cc5ccc(C(=O)N6CCN(CC7CCN(c8ccc(C9CCC(=O)NC9=O)cn8)CC7)CC6)cc5[nH]4)c3C[C@@H]1C2(F)F] | ITK |
| 21 | 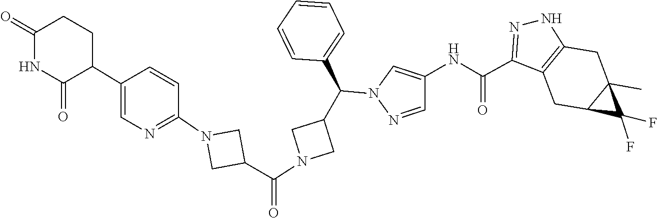 [SMILES: C[C@@]12Cc3[nH]nc(C(=O)Nc4cnn([C@H](c5ccccc5)C5CN(C(=O)C6CN(c7ccc(C8CCC(=O)NC8=O)cn7)C6)C5)c4)c3C[C@@H]1C2(F)F] | ITK |
| 22 | 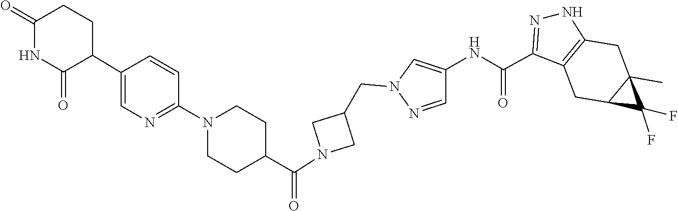 [SMILES: C[C@@]12Cc3[nH]nc(C(=O)Nc4cnn(CC5CN(C(=O)C6CCN(c7ccc(C8CCC(=O)NC8=O)cn7)CC6)C5)c4)c3C[C@@H]1C2(F)F] | ITK |
| 23 | 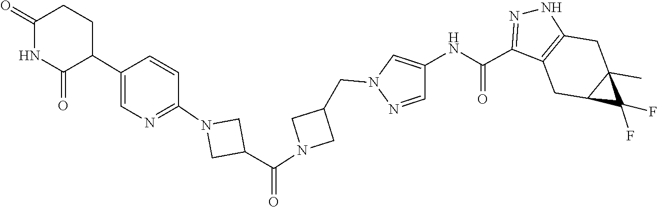 [SMILES: C[C@@]12Cc3[nH]nc(C(=O)Nc4cnn(CC5CN(C(=O)C6CN(c7ccc(C8CCC(=O)NC8=O)cn7)C6)C5)c4)c3C[C@@H]1C2(F)F] | ITK |
| 24 | 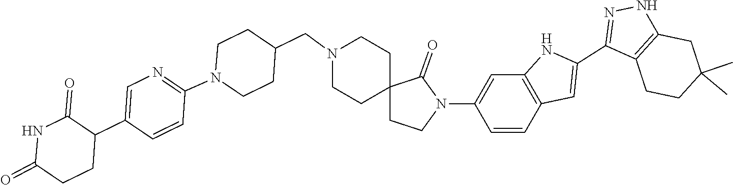 [SMILES: CC1(C)CCc2c(-c3cc4ccc(N5CCC6(CCN(CC7CCN(c8ccc(C9CCC(=O)NC9=O)cn8)CC7)CC6)C5=O)cc4[nH]3)n[nH]c2C1] | ITK |
| 25 | 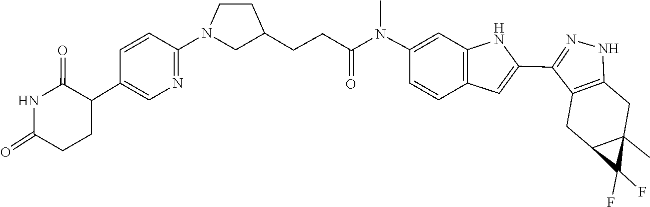 [SMILES: CN(C(=O)CCC1CCN(c2ccc(C3CCC(=O)NC3=O)cn2)C1)c1ccc2cc(-c3n[nH]c4c3C[C@@H]3C(F)(F)[C@]3(C)C4)[nH]c2c1] | ITK |
| 26 | 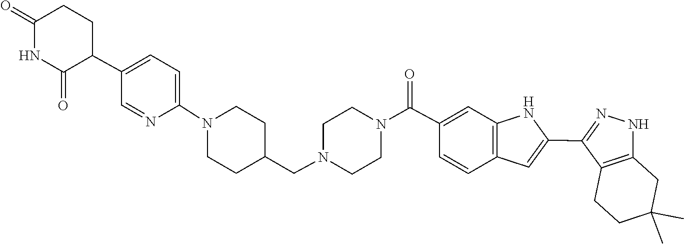 [SMILES: CC1(C)CCc2c(-c3cc4ccc(C(=O)N5CCN(CC6CCN(c7ccc(C8CCC(=O)NC8=O)cn7)CC6)CC5)cc4[nH]3)n[nH]c2C1] | ITK |
| 27 | 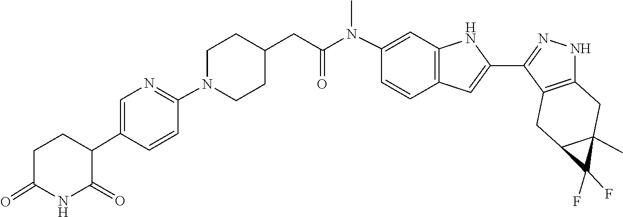 [SMILES: CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1] | ITK |
| 28 | 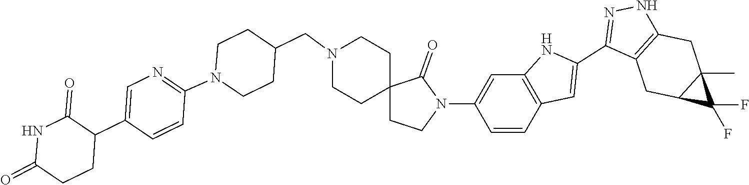 [SMILES: C[C@@]12Cc3[nH]nc(-c4cc5ccc(N6CCC7(CCN(CC8CCN(c9ccc(C%10CCC(=O)NC%10=O)cn9)CC8)CC7)C6=O)cc5[nH]4)c3C[C@@H]1C2(F)F] | ITK |
| 29 | 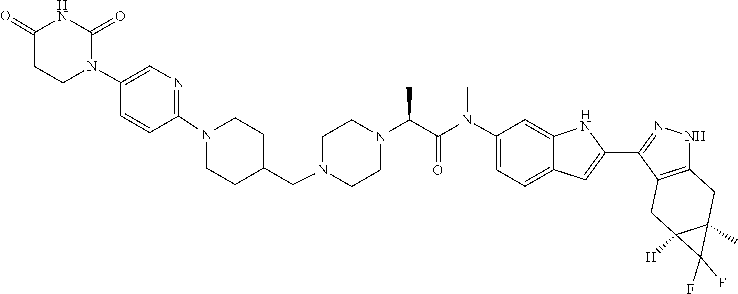 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(N4CCC(=O)NC4=O)cn3)CC2)CC1] | ITK |
| 30 | 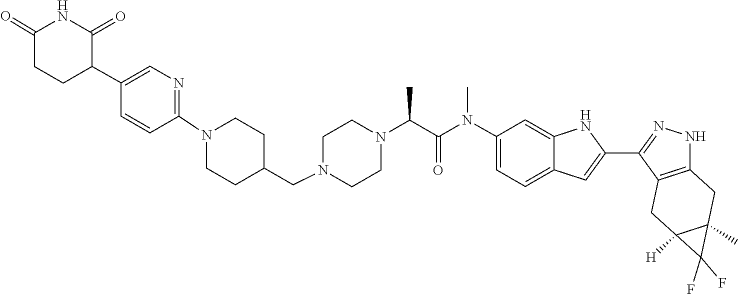 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(C4CCC(=O)NC4=O)cn3)CC2)CC1] | ITK |
| 31 | 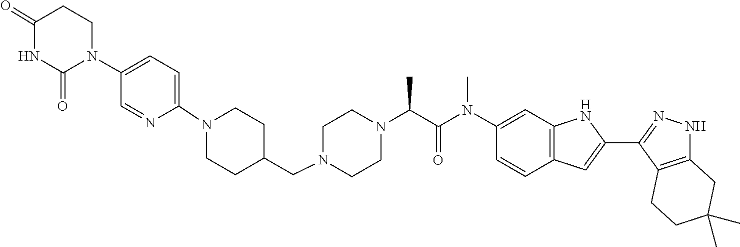 [SMILES: C[C@@H](C(=O)N(C)c1ccc2cc(-c3n[nH]c4c3CCC(C)(C)C4)[nH]c2c1)N1CCN(CC2CCN(c3ccc(N4CCC(=O)NC4=O)cn3)CC2)CC1] | ITK |
| 32 | 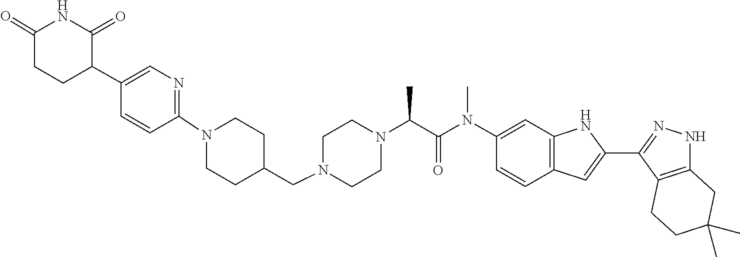 [SMILES: C[C@@H](C(=O)N(C)c1ccc2cc(-c3n[nH]c4c3CCC(C)(C)C4)[nH]c2c1)N1CCN(CC2CCN(c3ccc(C4CCC(=O)NC4=O)cn3)CC2)CC1] | ITK |
| 33 | 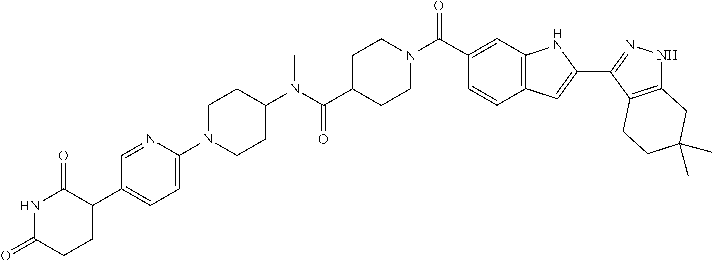 [SMILES: CN(C(=O)C1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1] | ITK |
| 34 | 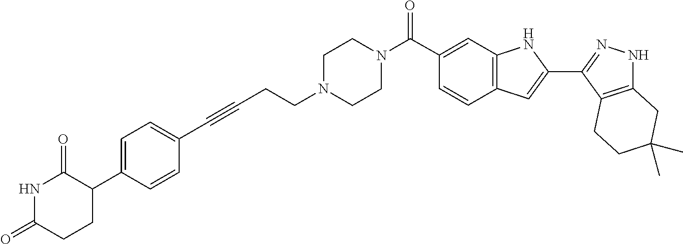 [SMILES: CC1(C)CCc2c(-c3cc4ccc(C(=O)N5CCN(CCC#Cc6ccc(C7CCC(=O)NC7=O)cc6)CC5)cc4[nH]3)n[nH]c2C1] | ITK |
| 35 | 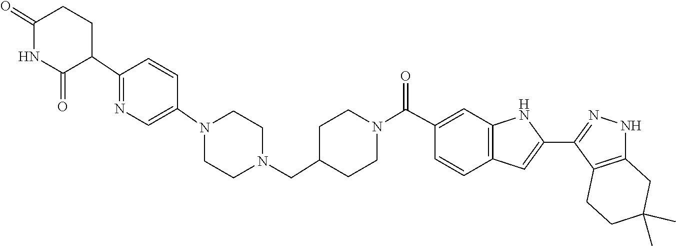 [SMILES: CC1(C)CCc2c(-c3cc4ccc(C(=O)N5CCC(CN6CCN(c7ccc(C8CCC(=O)NC8=O)nc7)CC6)CC5)cc4[nH]3)n[nH]c2C1] | ITK |
| 36 | 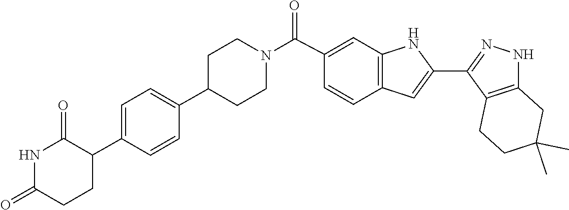 [SMILES: CC1(C)CCc2c(-c3cc4ccc(C(=O)N5CCC(c6ccc(C7CCC(=O)NC7=O)cc6)CC5)cc4[nH]3)n[nH]c2C1] | ITK |
| 37 | 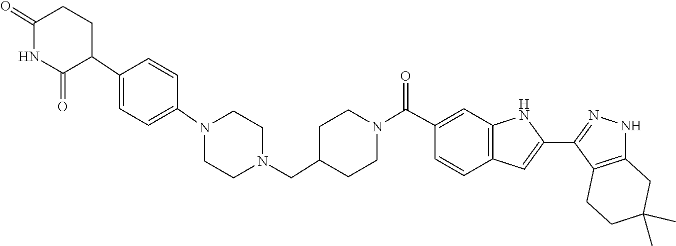 [SMILES: CC1(C)CCc2c(-c3cc4ccc(C(=O)N5CCC(CN6CCN(c7ccc(C8CCC(=O)NC8=O)cc7)CC6)CC5)cc4[nH]3)n[nH]c2C1] | ITK |
| 38 | 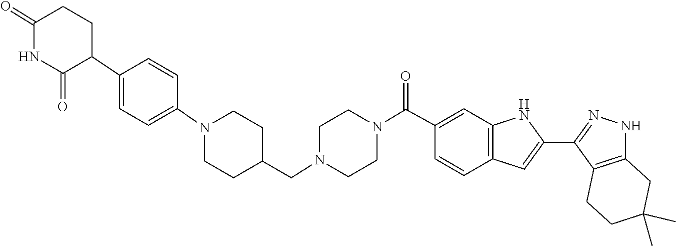 [SMILES: CC1(C)CCc2c(-c3cc4ccc(C(=O)N5CCN(CC6CCN(c7ccc(C8CCC(=O)NC8=O)cc7)CC6)CC5)cc4[nH]3)n[nH]c2C1] | ITK |
| 39 | 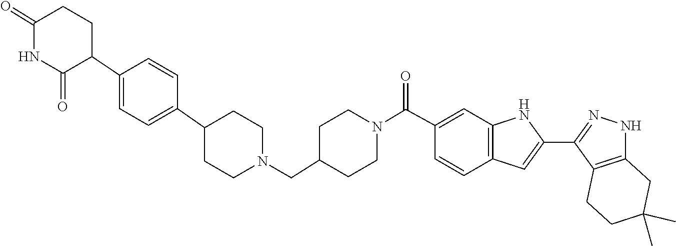 [SMILES: CC1(C)CCc2c(-c3cc4ccc(C(=O)N5CCC(CN6CCC(c7ccc(C8CCC(=O)NC8=O)cc7)CC6)CC5)cc4[nH]3)n[nH]c2C1] | ITK |
| 40 | 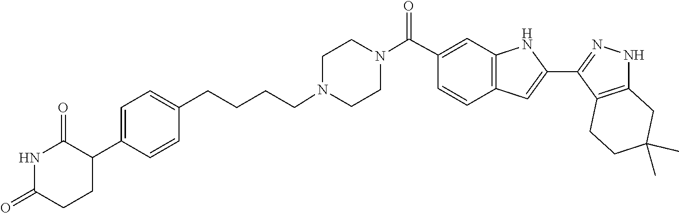 [SMILES: CC1(C)CCc2c(-c3cc4ccc(C(=O)N5CCN(CCCCc6ccc(C7CCC(=O)NC7=O)cc6)CC5)cc4[nH]3)n[nH]c2C1] | ITK |
| 41 | 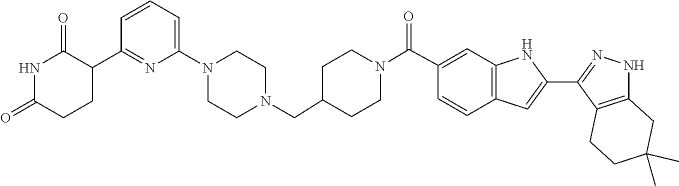 [SMILES: CC1(C)CCc2c(-c3cc4ccc(C(=O)N5CCC(CN6CCN(c7cccc(C8CCC(=O)NC8=O)n7)CC6)CC5)cc4[nH]3)n[nH]c2C1] | ITK |
| 42 | 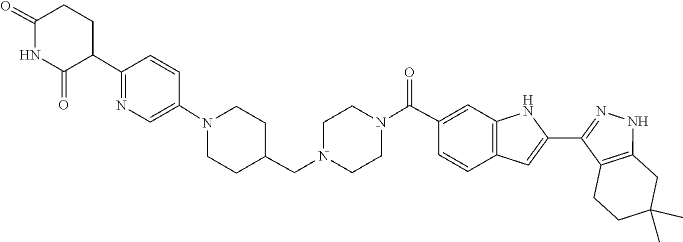 [SMILES: CC1(C)CCc2c(-c3cc4ccc(C(=O)N5CCN(CC6CCN(c7ccc(C8CCC(=O)NC8=O)nc7)CC6)CC5)cc4[nH]3)n[nH]c2C1] | ITK |
| 43 | 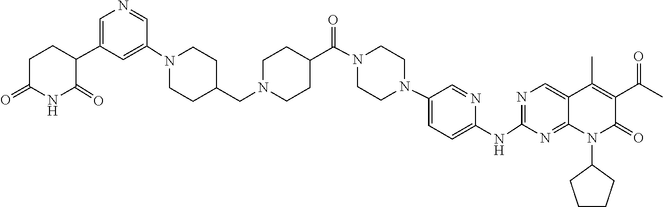 [SMILES: CC(=O)c1c(C)c2cnc(Nc3ccc(N4CCN(C(=O)C5CCN(CC6CCN(c7cncc(C8CCC(=O)NC8=O)c7)CC6)CC5)CC4)cn3)nc2n(C2CCCC2)c1=O] | CDK4 CDK6 |
| 44 | 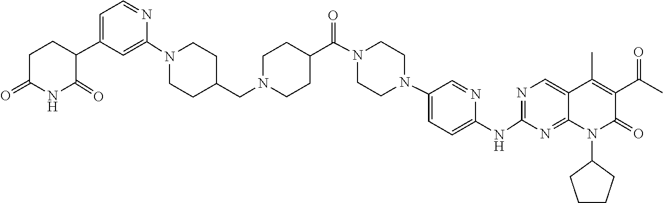 [SMILES: CC(=O)c1c(C)c2cnc(Nc3ccc(N4CCN(C(=O)C5CCN(CC6CCN(c7cc(C8CCC(=O)NC8=O)ccn7)CC6)CC5)CC4)cn3)nc2n(C2CCCC2)c1=O] | CDK4 CDK6 |
| 45 | 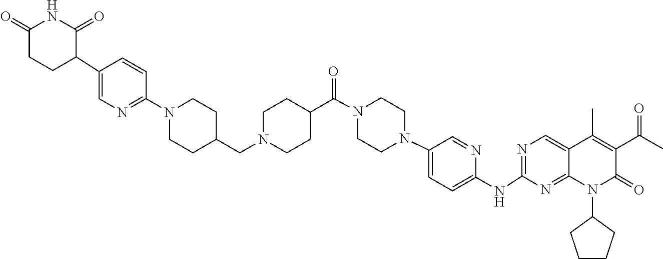 [SMILES: CC(=O)c1c(C)c2cnc(Nc3ccc(N4CCN(C(=O)C5CCN(CC6CCN(c7ccc(C8CCC(=O)NC8=O)cn7)CC6)CC5)CC4)cn3)nc2n(C2CCCC2)c1=O] | CDK4 CDK6 |
| 46 | 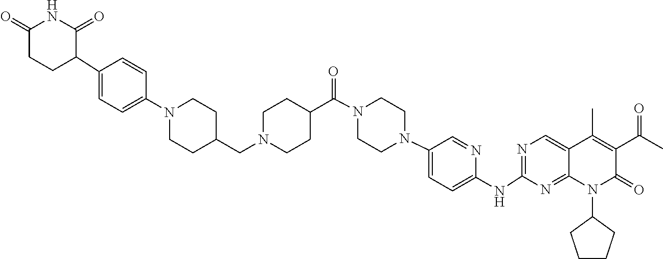 [SMILES: CC(=O)c1c(C)c2cnc(Nc3ccc(N4CCN(C(=O)C5CCN(CC6CCN(c7ccc(C8CCC(=O)NC8=O)cc7)CC6)CC5)CC4)cn3)nc2n(C2CCCC2)c1=O] | CDK4 CDK6 |
| 47 | 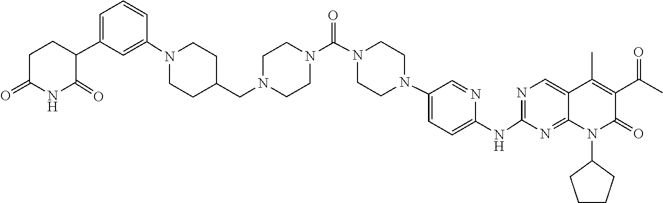 [SMILES: CC(=O)c1c(C)c2cnc(Nc3ccc(N4CCN(C(=O)N5CCN(CC6CCN(c7cccc(C8CCC(=O)NC8=O)c7)CC6)CC5)CC4)cn3)nc2n(C2CCCC2)c1=O] | CDK4 CDK6 |
| 48 | 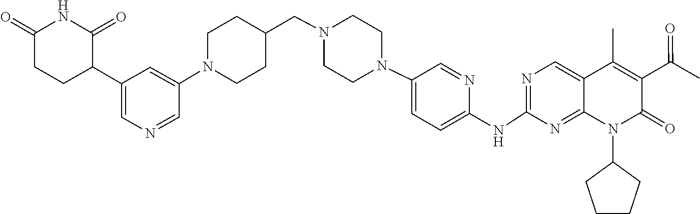 [SMILES: CC(=O)c1c(C)c2cnc(Nc3ccc(N4CCN(CC5CCN(c6cncc(C7CCC(=O)NC7=O)c6)CC5)CC4)cn3)nc2n(C2CCCC2)c1=O] | CDK4 CDK6 |
| 49 | 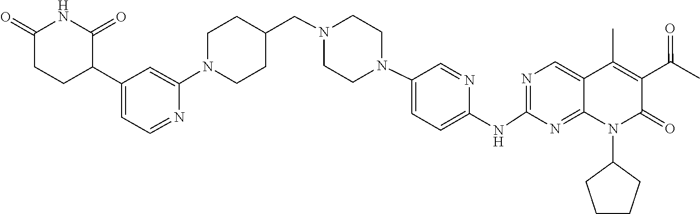 [SMILES: CC(=O)c1c(C)c2cnc(Nc3ccc(N4CCN(CC5CCN(c6cc(C7CCC(=O)NC7=O)ccn6)CC5)CC4)cn3)nc2n(C2CCCC2)c1=O] | CDK4 CDK6 |
| 50 | 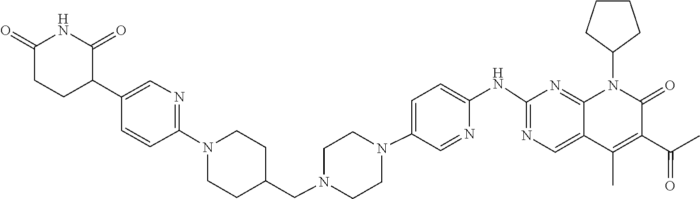 [SMILES: CC(=O)c1c(C)c2cnc(Nc3ccc(N4CCN(CC5CCN(c6ccc(C7CCC(=O)NC7=O)cn6)CC5)CC4)cn3)nc2n(C2CCCC2)c1=O] | CDK4 CDK6 |
| 51 | 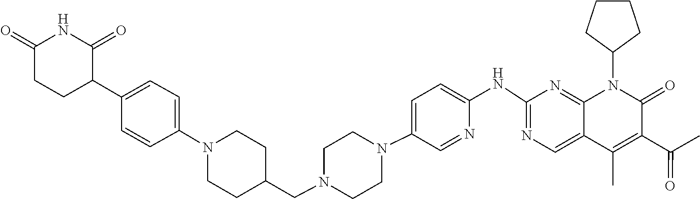 [SMILES: CC(=O)c1c(C)c2cnc(Nc3ccc(N4CCN(CC5CCN(c6ccc(C7CCC(=O)NC7=O)cc6)CC5)CC4)cn3)nc2n(C2CCCC2)c1=O] | CDK4 CDK6 |
| 52 | 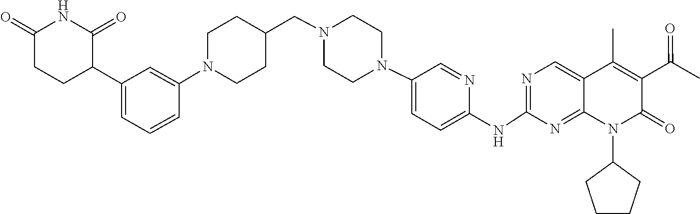 [SMILES: CC(=O)c1c(C)c2cnc(Nc3ccc(N4CCN(CC5CCN(c6cccc(C7CCC(=O)NC7=O)c6)CC5)CC4)cn3)nc2n(C2CCCC2)c1=O] | CDK4 CDK6 |
| 53 | 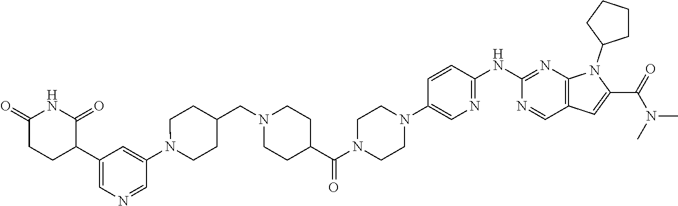 [SMILES: CN(C)C(=O)c1cc2cnc(Nc3ccc(N4CCN(C(=O)C5CCN(CC6CCN(c7cncc(C8CCC(=O)NC8=O)c7)CC6)CC5)CC4)cn3)nc2n1C1CCCC1] | CDK4 CDK6 |
| 54 | 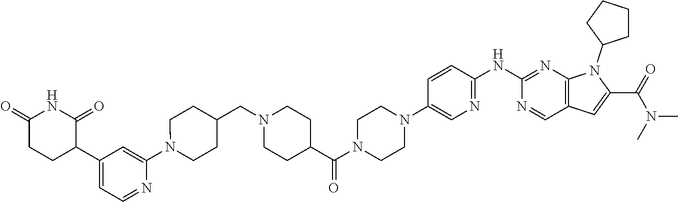 [SMILES: CN(C)C(=O)c1cc2cnc(Nc3ccc(N4CCN(C(=O)C5CCN(CC6CCN(c7cc(C8CCC(=O)NC8=O)ccn7)CC6)CC5)CC4)cn3)nc2n1C1CCCC1] | CDK4 CDK6 |
| 55 | 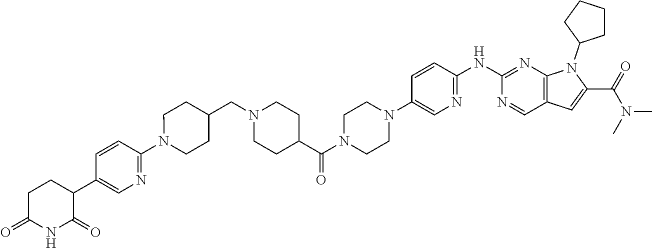 [SMILES: CN(C)C(=O)c1cc2cnc(Nc3ccc(N4CCN(C(=O)C5CCN(CC6CCN(c7ccc(C8CCC(=O)NC8=O)cn7)CC6)CC5)CC4)cn3)nc2n1C1CCCC1] | CDK4 CDK6 |
| 56 | 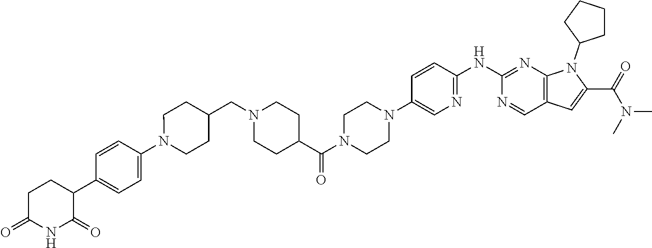 [SMILES: CN(C)C(=O)c1cc2cnc(Nc3ccc(N4CCN(C(=O)C5CCN(CC6CCN(c7ccc(C8CCC(=O)NC8=O)cc7)CC6)CC5)CC4)cn3)nc2n1C1CCCC1] | CDK4 CDK6 |
| 57 | 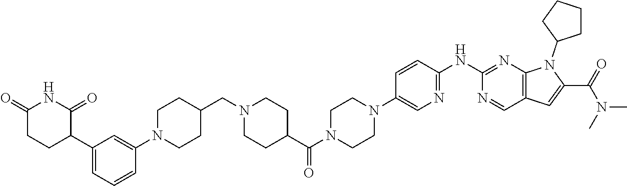 [SMILES: CN(C)C(=O)c1cc2cnc(Nc3ccc(N4CCN(C(=O)C5CCN(CC6CCN(c7cccc(C8CCC(=O)NC8=O)c7)CC6)CC5)CC4)cn3)nc2n1C1CCCC1] | CDK4 CDK6 |
| 58 | 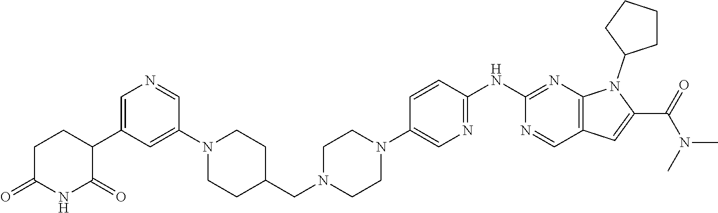 [SMILES: CN(C)C(=O)c1cc2cnc(Nc3ccc(N4CCN(CC5CCN(c6cncc(C7CCC(=O)NC7=O)c6)CC5)CC4)cn3)nc2n1C1CCCC1] | CDK4 CDK6 |
| 59 | 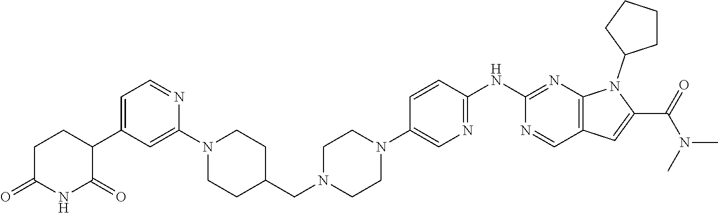 [SMILES: CN(C)C(=O)c1cc2cnc(Nc3ccc(N4CCN(CC5CCN(c6cc(C7CCC(=O)NC7=O)ccn6)CC5)CC4)cn3)nc2n1C1CCCC1] | CDK4 CDK6 |
| 60 | 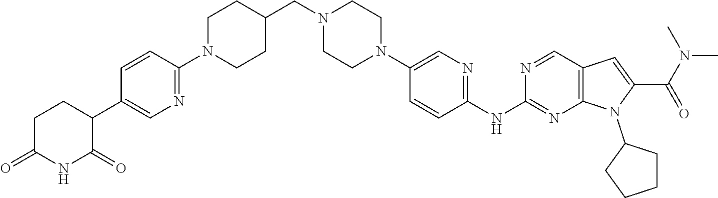 [SMILES: CN(C)C(=O)c1cc2cnc(Nc3ccc(N4CCN(CC5CCN(c6ccc(C7CCC(=O)NC7=O)cn6)CC5)CC4)cn3)nc2n1C1CCCC1] | CDK4 CDK6 |
| 61 | 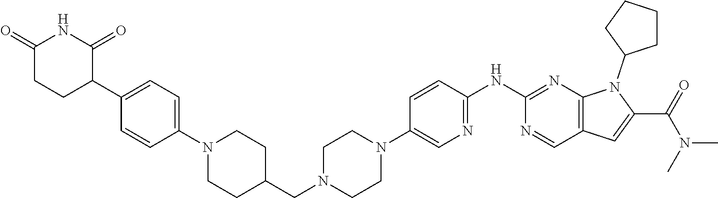 [SMILES: CN(C)C(=O)c1cc2cnc(Nc3ccc(N4CCN(CC5CCN(c6ccc(C7CCC(=O)NC7=O)cc6)CC5)CC4)cn3)nc2n1C1CCCC1] | CDK4 CDK6 |
| 62 | 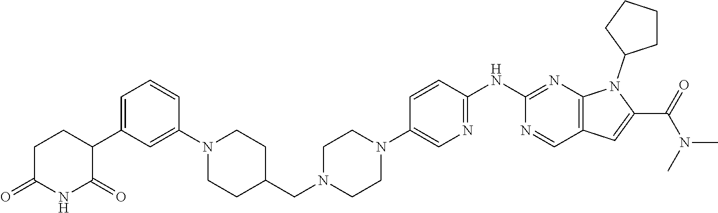 [SMILES: CN(C)C(=O)c1cc2cnc(Nc3ccc(N4CCN(CC5CCN(c6cccc(C7CCC(=O)NC7=O)c6)CC5)CC4)cn3)nc2n1C1CCCC1] | CDK4 CDK6 |
| 63 | 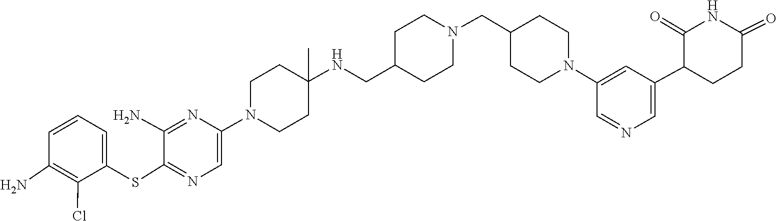 [SMILES: CC1(NCC2CCN(CC3CCN(c4cncc(C5CCC(=O)NC5=O)c4)CC3)CC2)CCN(c2cnc(Sc3cccc(N)c3Cl)c(N)n2)CC1] | SHP-2 |
| 64 | 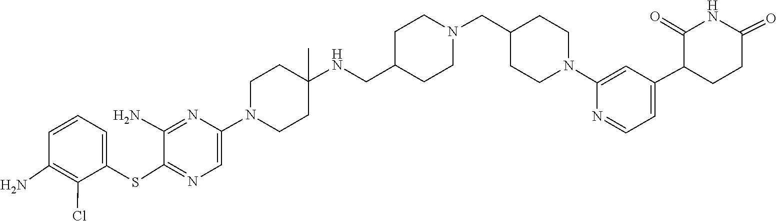 [SMILES: CC1(NCC2CCN(CC3CCN(c4cc(C5CCC(=O)NC5=O)ccn4)CC3)CC2)CCN(c2cnc(Sc3cccc(N)c3Cl)c(N)n2)CC1] | SHP-2 |
| 65 | 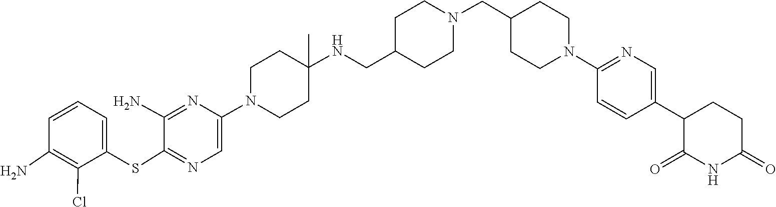 [SMILES: CC1(NCC2CCN(CC3CCN(c4ccc(C5CCC(=O)NC5=O)cn4)CC3)CC2)CCN(c2cnc(Sc3cccc(N)c3Cl)c(N)n2)CC1] | SHP-2 |
| 66 | 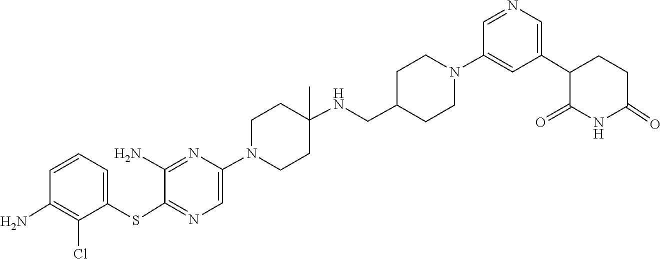 [SMILES: CC1(NCC2CCN(c3cncc(C4CCC(=O)NC4=O)c3)CC2)CCN(c2cnc(Sc3cccc(N)c3Cl)c(N)n2)CC1] | SHP-2 |
| 67 | 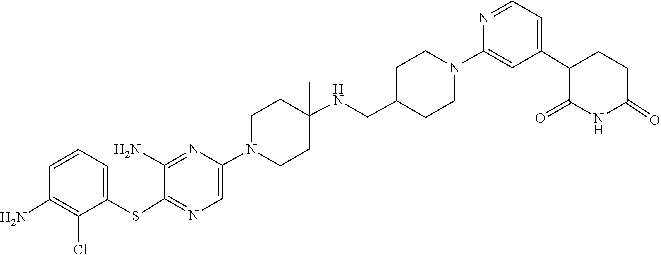 [SMILES: CC1(NCC2CCN(c3cc(C4CCC(=O)NC4=O)ccn3)CC2)CCN(c2cnc(Sc3cccc(N)c3Cl)c(N)n2)CC1] | SHP-2 |
| 68 | 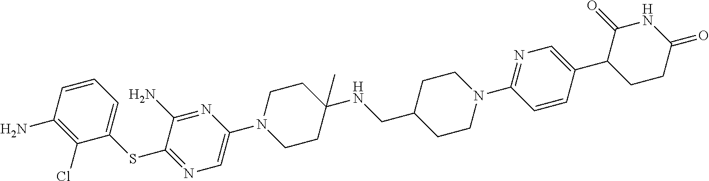 [SMILES: CC1(NCC2CCN(c3ccc(C4CCC(=O)NC4=O)cn3)CC2)CCN(c2cnc(Sc3cccc(N)c3Cl)c(N)n2)CC1] | SHP-2 |
| 69 | 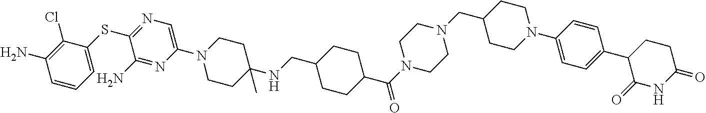 [SMILES: CC1(NCC2CCC(C(=O)N3CCN(CC4CCN(c5ccc(C6CCC(=O)NC6=O)cc5)CC4)CC3)CC2)CCN(c2cnc(Sc3cccc(N)c3Cl)c(N)n2)CC1] | SHP-2 |
| 70 | 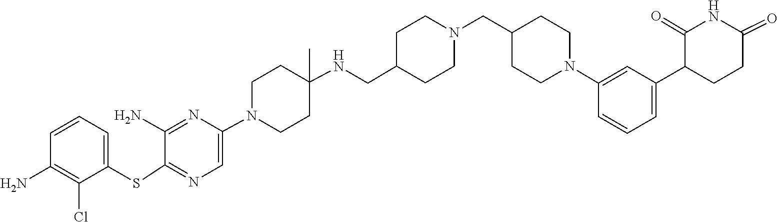 [SMILES: CC1(NCC2CCN(CC3CCN(c4cccc(C5CCC(=O)NC5=O)c4)CC3)CC2)CCN(c2cnc(Sc3cccc(N)c3Cl)c(N)n2)CC1] | SHP-2 |
| 71 | 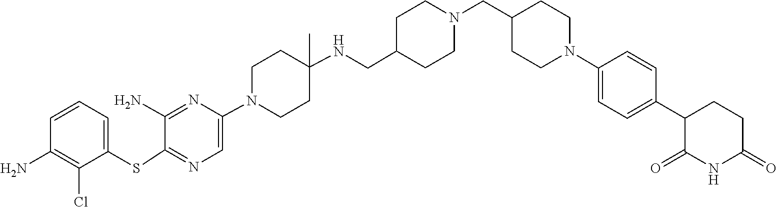 [SMILES: CC1(NCC2CCN(CC3CCN(c4ccc(C5CCC(=O)NC5=O)cc4)CC3)CC2)CCN(c2cnc(Sc3cccc(N)c3Cl)c(N)n2)CC1] | SHP-2 |
| 72 | 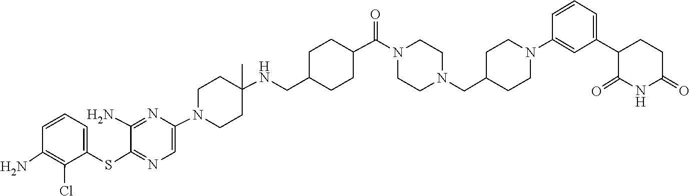 [SMILES: CC1(NCC2CCC(C(=O)N3CCN(CC4CCN(c5cccc(C6CCC(=O)NC6=O)c5)CC4)CC3)CC2)CCN(c2cnc(Sc3cccc(N)c3Cl)c(N)n2)CC1] | SHP-2 |
| 73 | 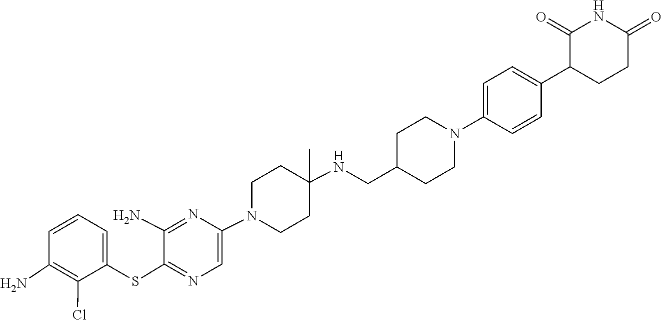 [SMILES: CC1(NCC2CCN(c3ccc(C4CCC(=O)NC4=O)cc3)CC2)CCN(c2cnc(Sc3cccc(N)c3Cl)c(N)n2)CC1] | SHP-2 |
| 74 | 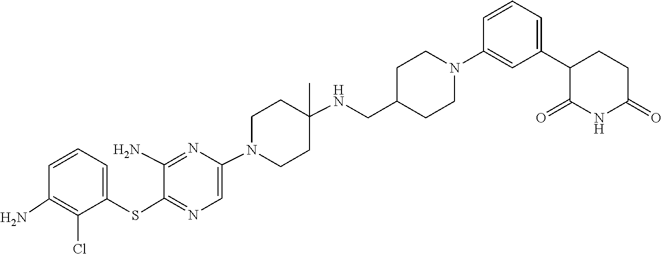 [SMILES: CC1(NCC2CCN(c3cccc(C4CCC(=O)NC4=O)c3)CC2)CCN(c2cnc(Sc3cccc(N)c3Cl)c(N)n2)CC1] | SHP-2 |
| 75 | 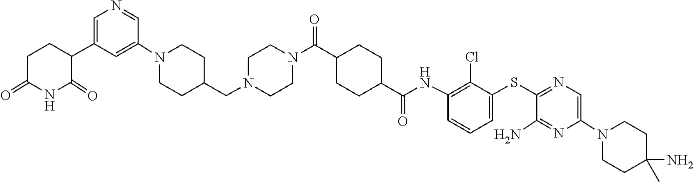 [SMILES: CC1(N)CCN(c2cnc(Sc3cccc(NC(=O)C4CCC(C(=O)N5CCN(CC6CCN(c7cncc(C8CCC(=O)NC8=O)c7)CC6)CC5)CC4)c3Cl)c(N)n2)CC1] | SHP-2 |
| 76 | 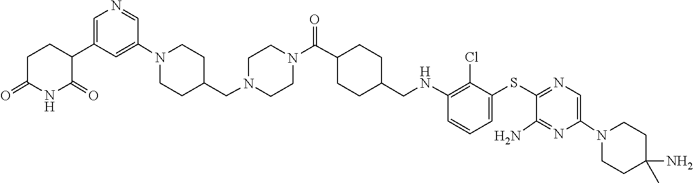 [SMILES: CC1(N)CCN(c2cnc(Sc3cccc(NCC4CCC(C(=O)N5CCN(CC6CCN(c7cncc(C8CCC(=O)NC8=O)c7)CC6)CC5)CC4)c3Cl)c(N)n2)CC1] | SHP-2 |
| 77 | 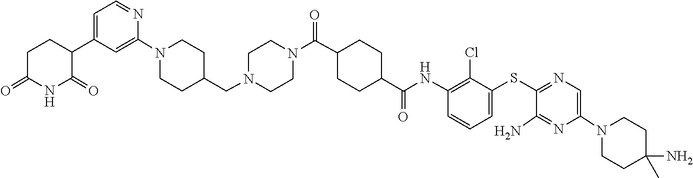 [SMILES: CC1(N)CCN(c2cnc(Sc3cccc(NC(=O)C4CCC(C(=O)N5CCN(CC6CCN(c7cc(C8CCC(=O)NC8=O)ccn7)CC6)CC5)CC4)c3Cl)c(N)n2)CC1] | SHP-2 |
| 78 | 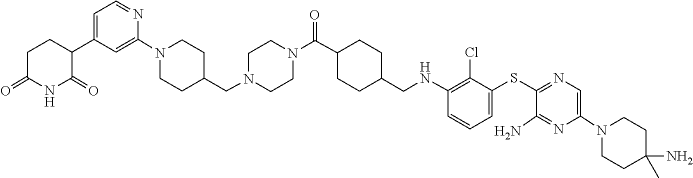 [SMILES: CC1(N)CCN(c2cnc(Sc3cccc(NCC4CCC(C(=O)N5CCN(CC6CCN(c7cc(C8CCC(=O)NC8=O)ccn7)CC6)CC5)CC4)c3Cl)c(N)n2)CC1] | SHP-2 |
| 79 | 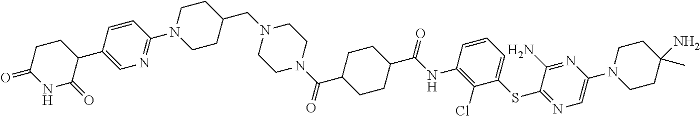 [SMILES: CC1(N)CCN(c2cnc(Sc3cccc(NC(=O)C4CCC(C(=O)N5CCN(CC6CCN(c7ccc(C8CCC(=O)NC8=O)cn7)CC6)CC5)CC4)c3Cl)c(N)n2)CC1] | SHP-2 |
| 80 | 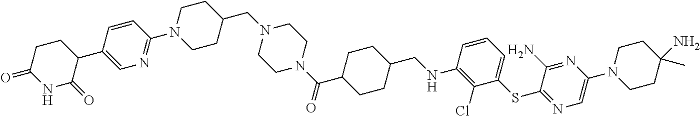 [SMILES: CC1(N)CCN(c2cnc(Sc3cccc(NCC4CCC(C(=O)N5CCN(CC6CCN(c7ccc(C8CCC(=O)NC8=O)cn7)CC6)CC5)CC4)c3Cl)c(N)n2)CC1] | SHP-2 |
| 81 | 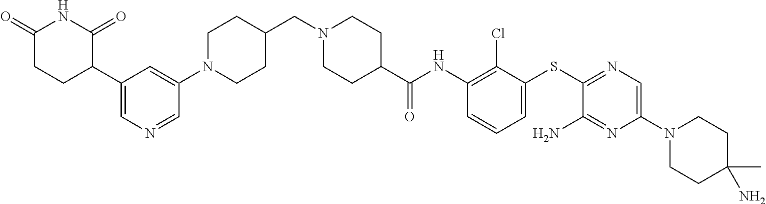 [SMILES: CC1(N)CCN(c2cnc(Sc3cccc(NC(=O)C4CCN(CC5CCN(c6cncc(C7CCC(=O)NC7=O)c6)CC5)CC4)c3Cl)c(N)n2)CC1] | SHP-2 |
| 82 | 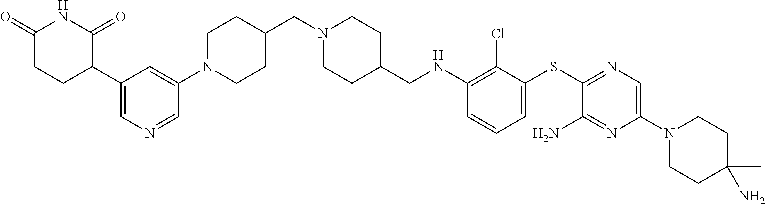 [SMILES: CC1(N)CCN(c2cnc(Sc3cccc(NCC4CCN(CC5CCN(c6cncc(C7CCC(=O)NC7=O)c6)CC5)CC4)c3Cl)c(N)n2)CC1] | SHP-2 |
| 83 | 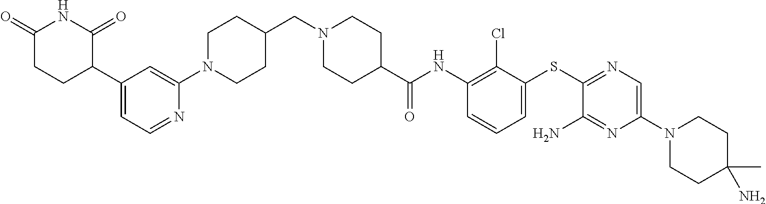 [SMILES: CC1(N)CCN(c2cnc(Sc3cccc(NC(=O)C4CCN(CC5CCN(c6cc(C7CCC(=O)NC7=O)ccn6)CC5)CC4)c3Cl)c(N)n2)CC1] | SHP-2 |
| 84 | 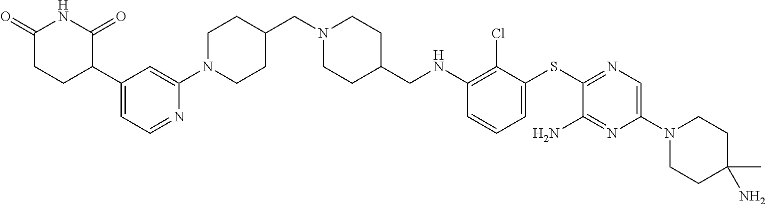 [SMILES: CC1(N)CCN(c2cnc(Sc3cccc(NCC4CCN(CC5CCN(c6cc(C7CCC(=O)NC7=O)ccn6)CC5)CC4)c3Cl)c(N)n2)CC1] | SHP-2 |
| 85 | 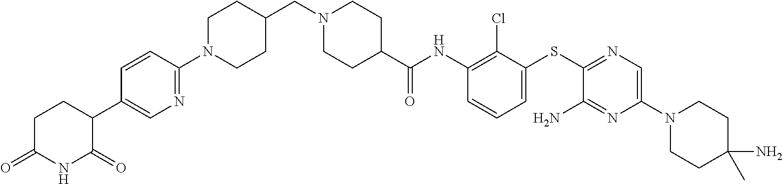 [SMILES: CC1(N)CCN(c2cnc(Sc3cccc(NC(=O)C4CCN(CC5CCN(c6ccc(C7CCC(=O)NC7=O)cn6)CC5)CC4)c3Cl)c(N)n2)CC1] | SHP-2 |
| 86 | 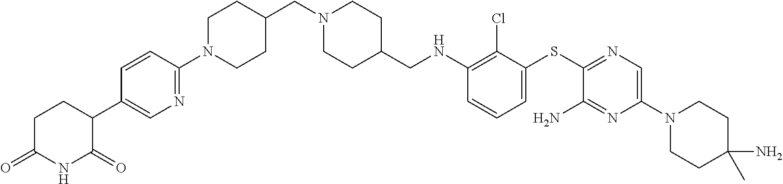 [SMILES: CC1(N)CCN(c2cnc(Sc3cccc(NCC4CCN(CC5CCN(c6ccc(C7CCC(=O)NC7=O)cn6)CC5)CC4)c3Cl)c(N)n2)CC1] | SHP-2 |
| 87 | 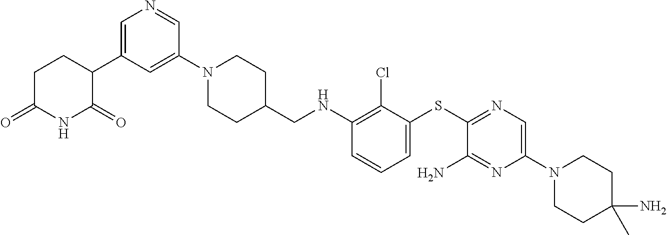 [SMILES: CC1(N)CCN(c2cnc(Sc3cccc(NCC4CCN(c5cncc(C6CCC(=O)NC6=O)c5)CC4)c3Cl)c(N)n2)CC1] | SHP-2 |
| 88 | 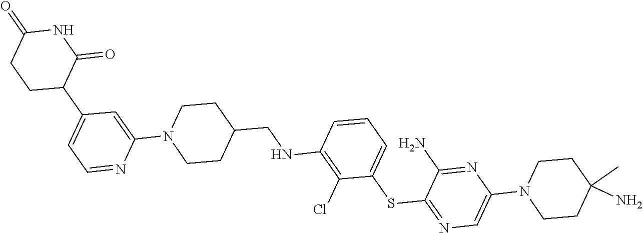 [SMILES: CC1(N)CCN(c2cnc(Sc3cccc(NCC4CCN(c5cc(C6CCC(=O)NC6=O)ccn5)CC4)c3Cl)c(N)n2)CC1] | SHP-2 |
| 89 | 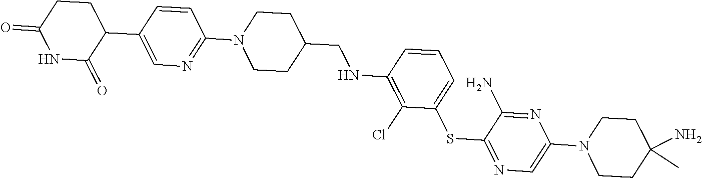 [SMILES: CC1(N)CCN(c2cnc(Sc3cccc(NCC4CCN(c5ccc(C6CCC(=O)NC6=O)cn5)CC4)c3Cl)c(N)n2)CC1] | SHP-2 |
| 90 | 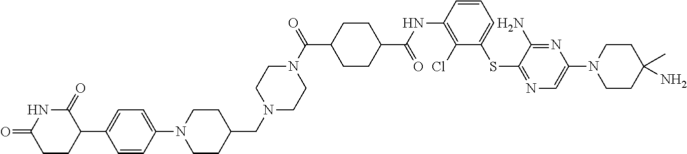 [SMILES: CC1(N)CCN(c2cnc(Sc3cccc(NC(=O)C4CCC(C(=O)N5CCN(CC6CCN(c7ccc(C8CCC(=O)NC8=O)cc7)CC6)CC5)CC4)c3Cl)c(N)n2)CC1] | SHP-2 |
| 91 | 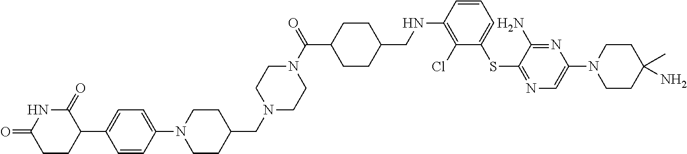 [SMILES: CC1(N)CCN(c2cnc(Sc3cccc(NCC4CCC(C(=O)N5CCN(CC6CCN(c7ccc(C8CCC(=O)NC8=O)cc7)CC6)CC5)CC4)c3Cl)c(N)n2)CC1] | SHP-2 |
| 92 | 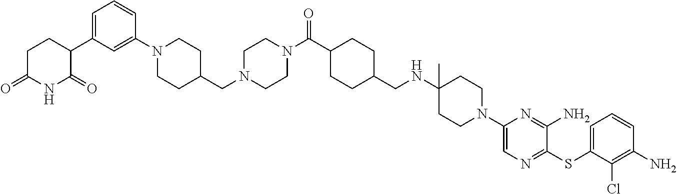 [SMILES: CC1(NCC2CCC(C(=O)N3CCN(CC4CCN(c5cccc(C6CCC(=O)NC6=O)c5)CC4)CC3)CC2)CCN(c2cnc(Sc3cccc(N)c3Cl)c(N)n2)CC1] | SHP-2 |
| 93 | 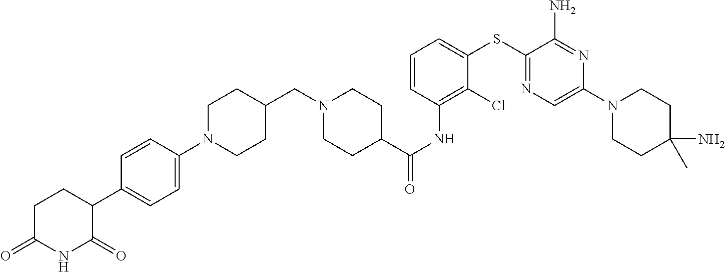 [SMILES: CC1(N)CCN(c2cnc(Sc3cccc(NC(=O)C4CCN(CC5CCN(c6ccc(C7CCC(=O)NC7=O)cc6)CC5)CC4)c3Cl)c(N)n2)CC1] | SHP-2 |
| 94 | 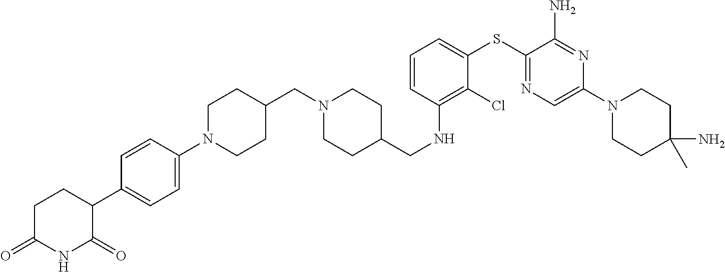 [SMILES: CC1(N)CCN(c2cnc(Sc3cccc(NCC4CCN(CC5CCN(c6ccc(C7CCC(=O)NC7=O)cc6)CC5)CC4)c3Cl)c(N)n2)CC1] | SHP-2 |
| 95 | 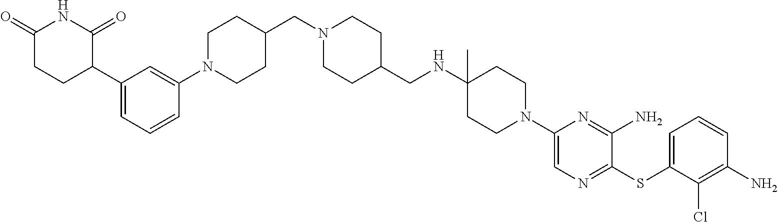 [SMILES: CC1(NCC2CCN(CC3CCN(c4cccc(C5CCC(=O)NC5=O)c4)CC3)CC2)CCN(c2cnc(Sc3cccc(N)c3Cl)c(N)n2)CC1] | SHP-2 |
| 96 | 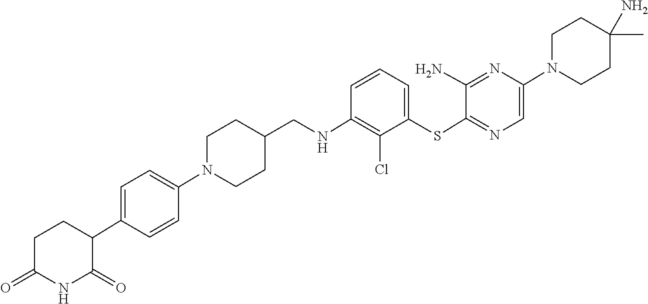 [SMILES: CC1(N)CCN(c2cnc(Sc3cccc(NCC4CCN(c5ccc(C6CCC(=O)NC6=O)cc5)CC4)c3Cl)c(N)n2)CC1] | SHP-2 |
| 97 | 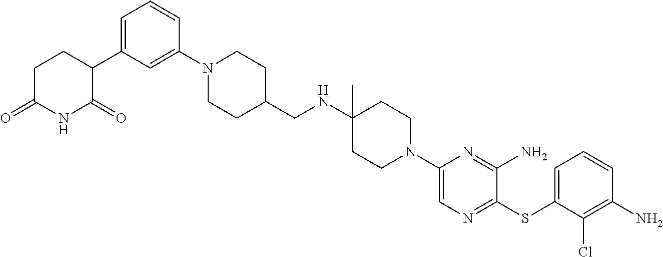 [SMILES: CC1(NCC2CCN(c3cccc(C4CCC(=O)NC4=O)c3)CC2)CCN(c2cnc(Sc3cccc(N)c3Cl)c(N)n2)CC1] | SHP-2 |
| 98 | 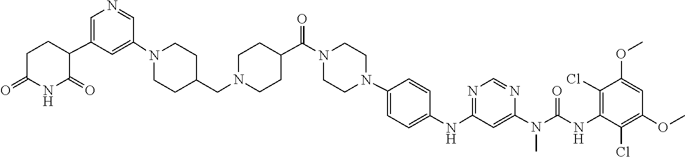 [SMILES: COc1cc(OC)c(Cl)c(NC(=O)N(C)c2cc(Nc3ccc(N4CCN(C(=O)C5CCN(CC6CCN(c7cncc(C8CCC(=O)NC8=O)c7)CC6)CC5)CC4)cc3)ncn2)c1Cl] | FGFR1 Fusion FGFR3 Fusion |
| 99 | 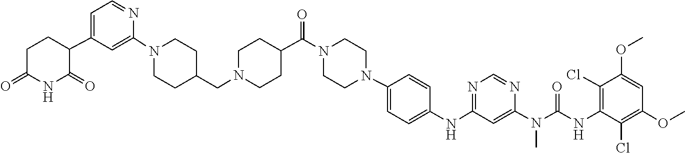 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] | FGFR1 Fusion FGFR3 Fusion |
| 100 | 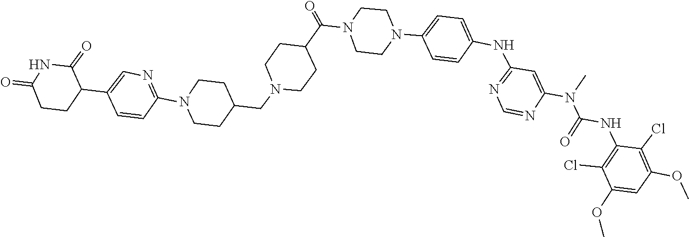 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cn7)CC6)CC5)CC4)cc3)ncn2)c1Cl] | FGFR1 Fusion FGFR3 Fusion |
| 101 | 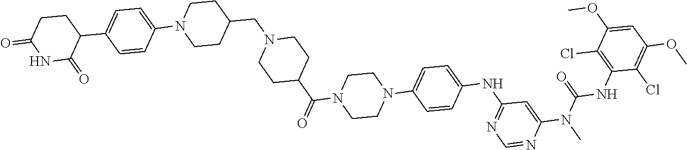 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cc7)CC6)CC5)CC4)cc3)ncn2)c1Cl] | FGFR1 Fusion FGFR3 Fusion |
| 102 | 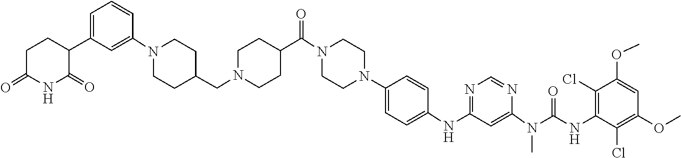 [SMILES: COc1cc(OC)c(Cl)c(NC(=O)N(C)c2cc(Nc3ccc(N4CCN(C(=O)C5CCN(CC6CCN(c7cccc(C8CCC(=O)NC8=O)c7)CC6)CC5)CC4)cc3)ncn2)c1Cl] | FGFR1 Fusion FGFR3 Fusion |
| 103 | 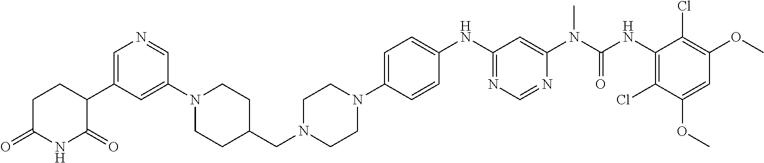 [SMILES: COc1cc(OC)c(Cl)c(NC(=O)N(C)c2cc(Nc3ccc(N4CCN(CC5CCN(c6cncc(C7CCC(=O)NC7=O)c6)CC5)CC4)cc3)ncn2)c1Cl] | FGFR1 Fusion FGFR3 Fusion |
| 104 | 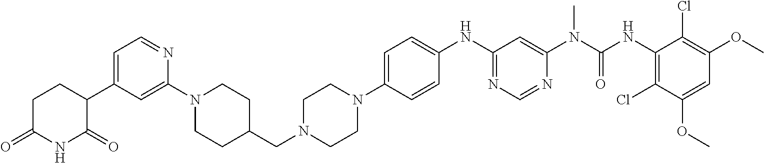 [SMILES: COc1cc(OC)c(Cl)c(NC(=O)N(C)c2cc(Nc3ccc(N4CCN(CC5CCN(c6cc(C7CCC(=O)NC7=O)ccn6)CC5)CC4)cc3)ncn2)c1Cl] | FGFR1 Fusion FGFR3 Fusion |
| 105 | 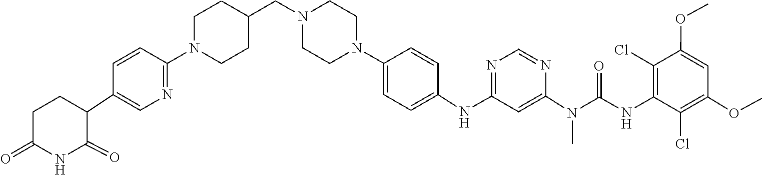 [SMILES: CCN(CCN(C)CC(C)CCN(CC)c1ccc(C2CCC(=O)NC2=O)cn1)c1ccc(Nc2cc(N(C)C(=O)Nc3c(Cl)c(OC)cc(OC)c3Cl)ncn2)cc1] | FGFR1 Fusion FGFR3 Fusion |
| 106 | 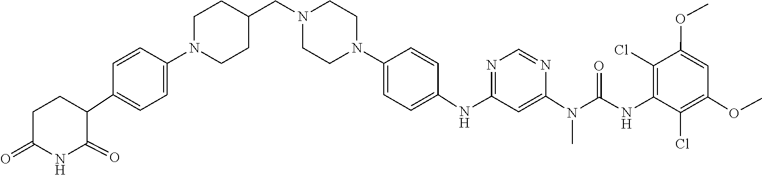 [SMILES: CCN(CCC(C)CN(C)CCN(CC)c1ccc(Nc2cc(N(C)C(=O)Nc3c(Cl)c(OC)cc(OC)c3Cl)ncn2)cc1)c1ccc(C2CCC(=O)NC2=O)cc1] | FGFR1 Fusion FGFR3 Fusion |
| 107 | 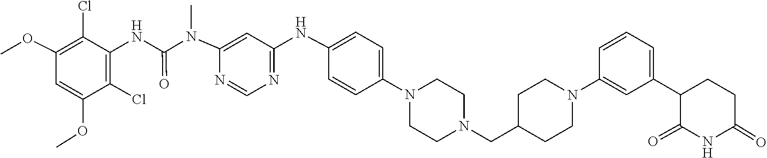 [SMILES: COc1cc(OC)c(Cl)c(NC(=O)N(C)c2cc(Nc3ccc(N4CCN(CC5CCN(c6cccc(C7CCC(=O)NC7=O)c6)CC5)CC4)cc3)ncn2)c1Cl] | FGFR1 Fusion FGFR3 Fusion |
| 108 | 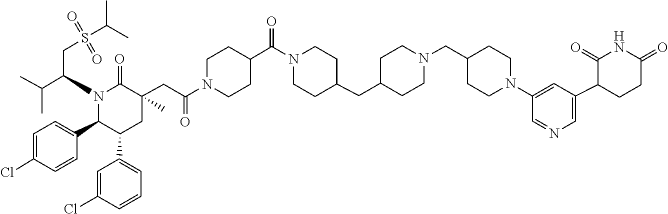 [SMILES: CC(C)[C@@H](CS(=O)(=O)C(C)C)N1C(=O)[C@@](C)(CC(=O)N2CCC(C(=O)N3CCC(CC4CCN(CC5CCN(c6cncc(C7CCC(=O)NC7=O)c6)CC5)CC4)CC3)CC2)C[C@H](c2cccc(Cl)c2)[C@H]1c1ccc(Cl)cc1] | MDM2 |
| 109 | 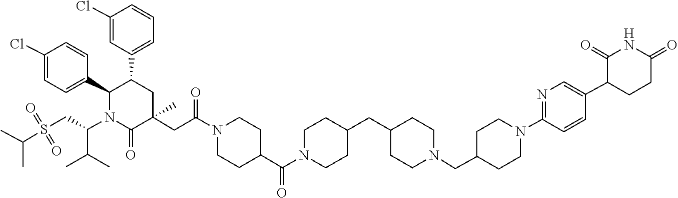 [SMILES: CC(C)[C@H](CS(=O)(=O)C(C)C)N1C(=O)[C@](C)(CC(=O)N2CCC(C(=O)N3CCC(CC4CCN(CC5CCN(c6ccc(C7CCC(=O)NC7=O)cn6)CC5)CC4)CC3)CC2)C[C@@H](c2cccc(Cl)c2)[C@@H]1c1ccc(Cl)cc1] | MDM2 |
| 110 | 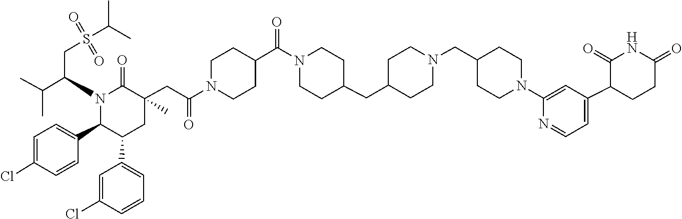 [SMILES: CC(C)[C@@H](CS(=O)(=O)C(C)C)N1C(=O)[C@@](C)(CC(=O)N2CCC(C(=O)N3CCC(CC4CCN(CC5CCN(c6cc(C7CCC(=O)NC7=O)ccn6)CC5)CC4)CC3)CC2)C[C@H](c2cccc(Cl)c2)[C@H]1c1ccc(Cl)cc1] | MDM2 |
| 111 | 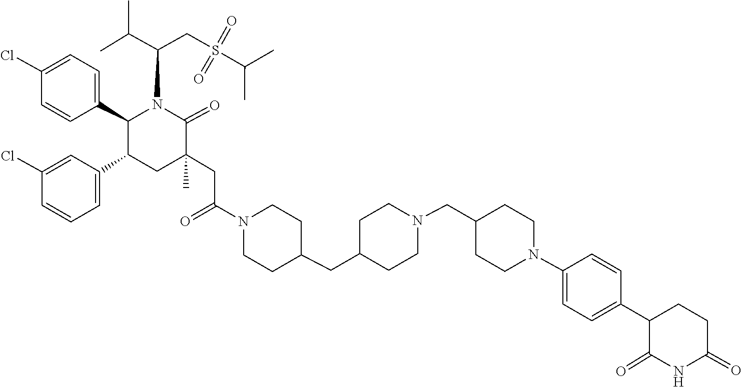 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cc5)CC4)CC3)CC2)C[C@H](c2cccc(Cl)c2)[C@H]1c1ccc(Cl)cc1] | MDM2 |
| 112 | 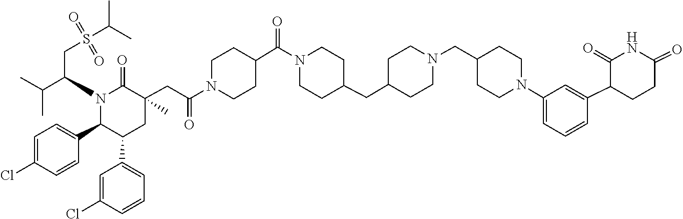 [SMILES: CC(C)[C@@H](CS(=O)(=O)C(C)C)N1C(=O)[C@@](C)(CC(=O)N2CCC(C(=O)N3CCC(CC4CCN(CC5CCN(c6cccc(C7CCC(=O)NC7=O)c6)CC5)CC4)CC3)CC2)C[C@H](c2cccc(Cl)c2)[C@H]1c1ccc(Cl)cc1] | MDM2 |
| 113 | 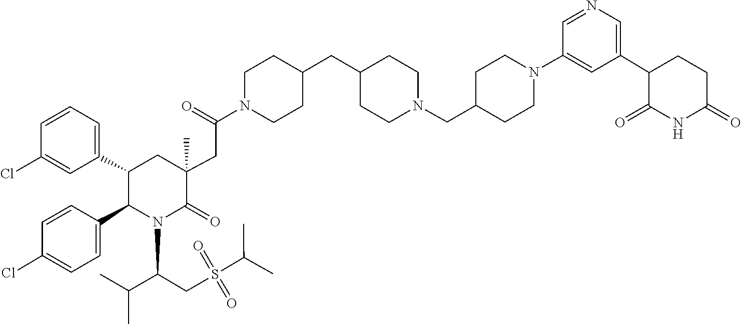 [SMILES: CC(C)[C@H](CS(=O)(=O)C(C)C)N1C(=O)[C@](C)(CC(=O)N2CCC(CC3CCN(CC4CCN(c5cncc(C6CCC(=O)NC6=O)c5)CC4)CC3)CC2)C[C@@H](c2cccc(Cl)c2)[C@@H]1c1ccc(Cl)cc1] | MDM2 |
| 114 | 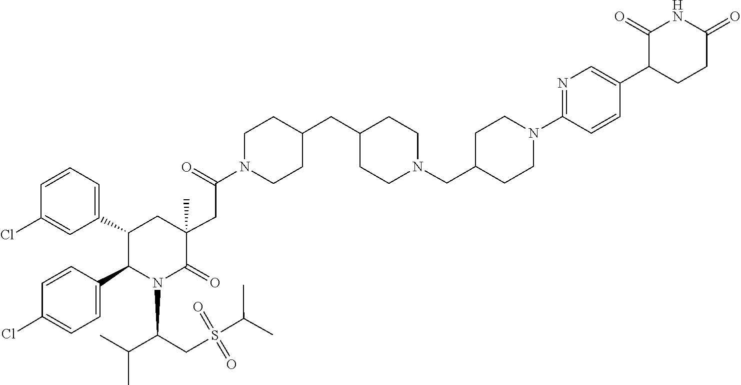 [SMILES: CC(C)[C@H](CS(=O)(=O)C(C)C)N1C(=O)[C@](C)(CC(=O)N2CCC(CC3CCN(CC4CCN(c5ccc(C6CCC(=O)NC6=O)cn5)CC4)CC3)CC2)C[C@@H](c2cccc(Cl)c2)[C@@H]1c1ccc(Cl)cc1] | MDM2 |
| 115 | 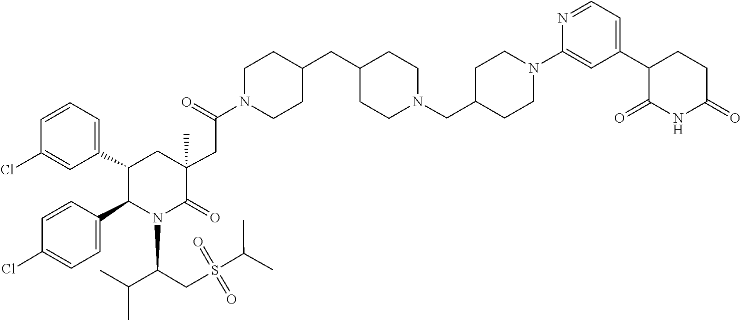 [SMILES: CC(C)[C@H](CS(=O)(=O)C(C)C)N1C(=O)[C@](C)(CC(=O)N2CCC(CC3CCN(CC4CCN(c5cc(C6CCC(=O)NC6=O)ccn5)CC4)CC3)CC2)C[C@@H](c2cccc(Cl)c2)[C@@H]1c1ccc(Cl)cc1] | MDM2 |
| 116 | 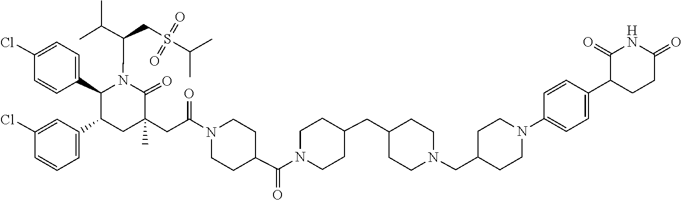 [SMILES: CC(C)[C@@H](CS(=O)(=O)C(C)C)N1C(=O)[C@@](C)(CC(=O)N2CCC(C(=O)N3CCC(CC4CCN(CC5CCN(c6ccc(C7CCC(=O)NC7=O)cc6)CC5)CC4)CC3)CC2)C[C@H](c2cccc(Cl)c2)[C@H]1c1ccc(Cl)cc1] | MDM2 |
| 117 | 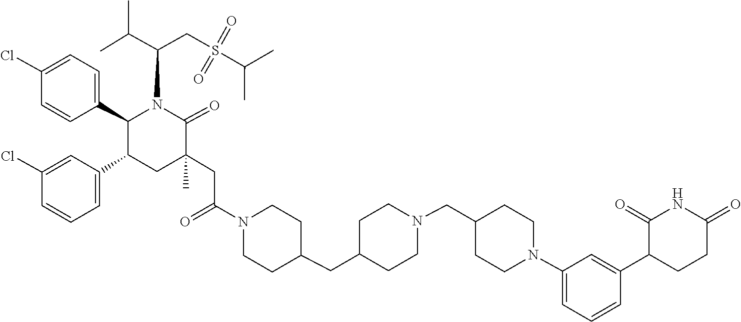 [SMILES: CC(C)[C@@H](CS(=O)(=O)C(C)C)N1C(=O)[C@@](C)(CC(=O)N2CCC(CC3CCN(CC4CCN(c5cccc(C6CCC(=O)NC6=O)c5)CC4)CC3)CC2)C[C@H](c2cccc(Cl)c2)[C@H]1c1ccc(Cl)cc1] | MDM2 |
| 118 | 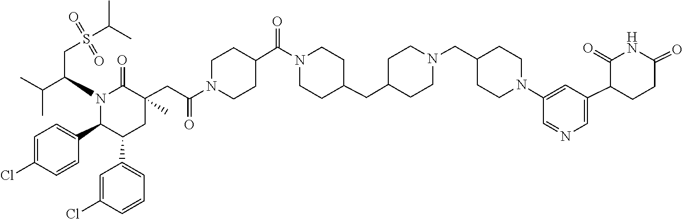 [SMILES: CC(C)[C@@H](CS(=O)(=O)C(C)C)N1C(=O)[C@@](C)(CC(=O)N2CCC(C(=O)N3CCC(CC4CCN(CC5CCN(c6cncc(C7CCC(=O)NC7=O)c6)CC5)CC4)CC3)CC2)C[C@H](c2cccc(Cl)c2)[C@H]1c1ccc(Cl)cc1] | MDM2 |
| 119 | 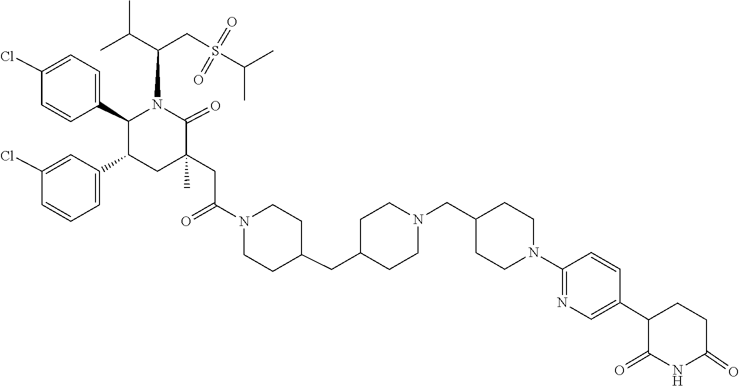 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cn5)CC4)CC3)CC2)C[C@H](c2cccc(Cl)c2)[C@H]1c1ccc(Cl)cc1] | MDM2 |
| 120 | 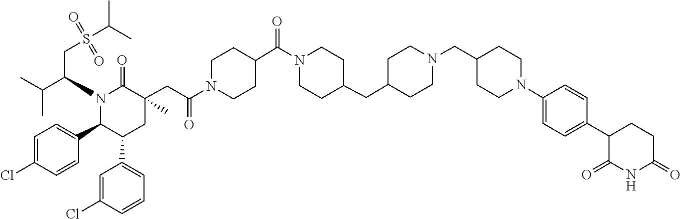 [SMILES: CC(C)[C@@H](CS(=O)(=O)C(C)C)N1C(=O)[C@@](C)(CC(=O)N2CCC(C(=O)N3CCC(CC4CCN(CC5CCN(c6ccc(C7CCC(=O)NC7=O)cc6)CC5)CC4)CC3)CC2)C[C@H](c2cccc(Cl)c2)[C@H]1c1ccc(Cl)cc1] | MDM2 |
| 121 | 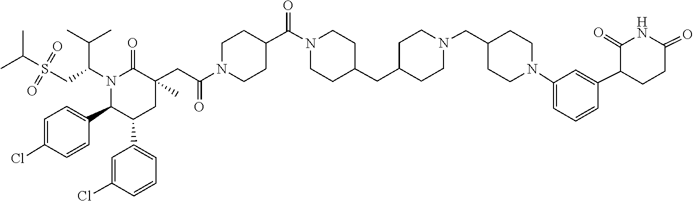 [SMILES: CC(C)[C@@H](CS(=O)(=O)C(C)C)N1C(=O)[C@@](C)(CC(=O)N2CCC(C(=O)N3CCC(CC4CCN(CC5CCN(c6cccc(C7CCC(=O)NC7=O)c6)CC5)CC4)CC3)CC2)C[C@H](c2cccc(Cl)c2)[C@H]1c1ccc(Cl)cc1] | MDM2 |
| 122 | 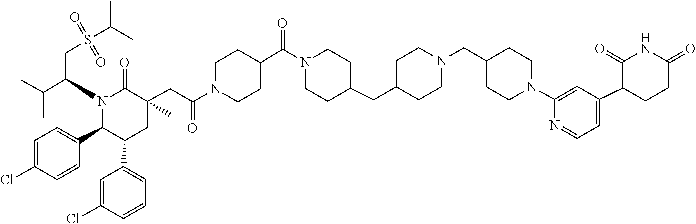 [SMILES: CC(C)[C@@H](CS(=O)(=O)C(C)C)N1C(=O)[C@@](C)(CC(=O)N2CCC(C(=O)N3CCC(CC4CCN(CC5CCN(c6cc(C7CCC(=O)NC7=O)ccn6)CC5)CC4)CC3)CC2)C[C@H](c2cccc(Cl)c2)[C@H]1c1ccc(Cl)cc1] | MDM2 |
| 123 | 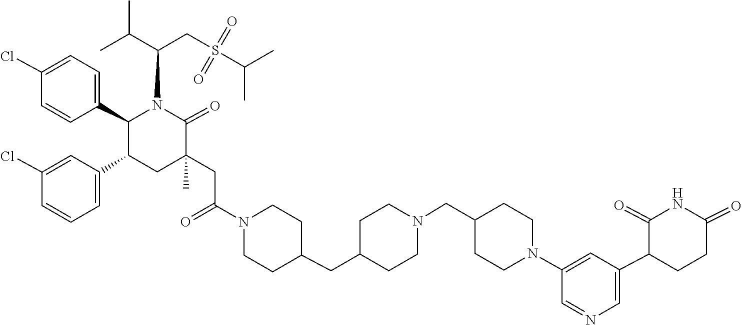 [SMILES: CC(C)[C@@H](CS(=O)(=O)C(C)C)N1C(=O)[C@@](C)(CC(=O)N2CCC(CC3CCN(CC4CCN(c5cncc(C6CCC(=O)NC6=O)c5)CC4)CC3)CC2)C[C@H](c2cccc(Cl)c2)[C@H]1c1ccc(Cl)cc1] | MDM2 |
| 124 | 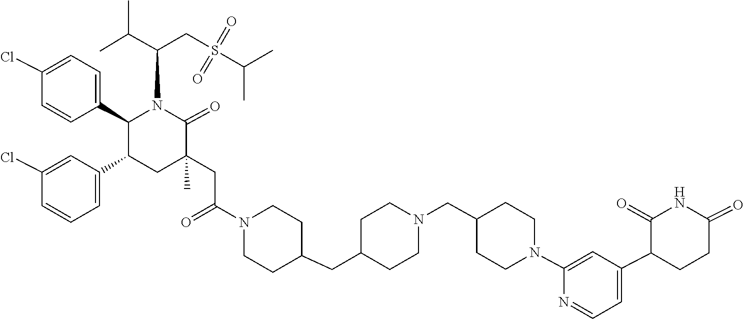 [SMILES: CC(C)[C@@H](CS(=O)(=O)C(C)C)N1C(=O)[C@@](C)(CC(=O)N2CCC(CC3CCN(CC4CCN(c5cc(C6CCC(=O)NC6=O)ccn5)CC4)CC3)CC2)C[C@H](c2cccc(Cl)c2)[C@H]1c1ccc(Cl)cc1] | MDM2 |
| 125 | 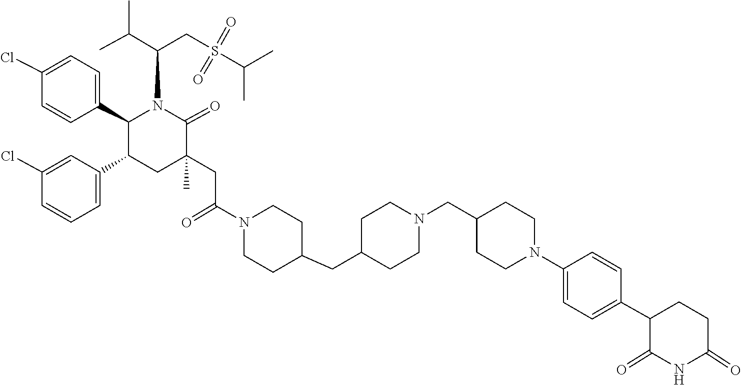 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cc5)CC4)CC3)CC2)C[C@H](c2cccc(Cl)c2)[C@H]1c1ccc(Cl)cc1] | MDM2 |
| 126 | 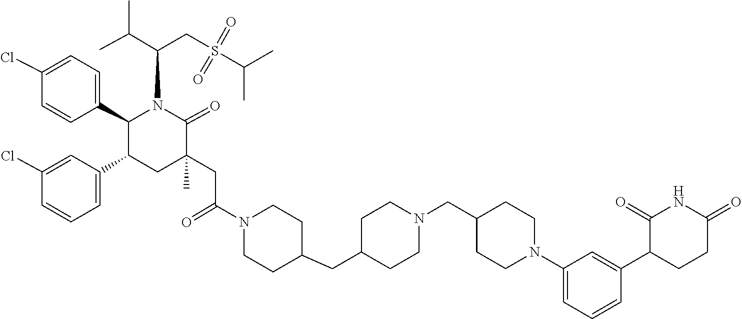 [SMILES: CC(C)[C@@H](CS(=O)(=O)C(C)C)N1C(=O)[C@@](C)(CC(=O)N2CCC(CC3CCN(CC4CCN(c5cccc(C6CCC(=O)NC6=O)c5)CC4)CC3)CC2)C[C@H](c2cccc(Cl)c2)[C@H]1c1ccc(Cl)cc1] | MDM2 |
| 127 | 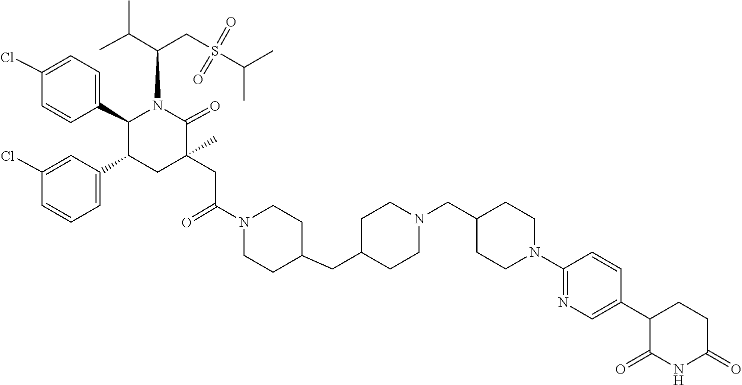 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cn5)CC4)CC3)CC2)C[C@H](c2cccc(Cl)c2)[C@H]1c1ccc(Cl)cc1] | MDM2 |
| 128 | 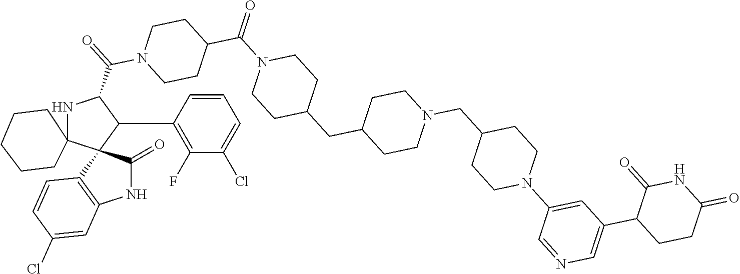 [SMILES: O=C1CCC(c2cncc(N3CCC(CN4CCC(CC5CCN(C(=O)C6CCN(C(=O)[C@H]7NC8(CCCCC8)[C@]8(C(=O)Nc9cc(Cl)ccc98)C7c7cccc(Cl)c7F)CC6)CC5)CC4)CC3)c2)C(=O)N1] | MDM2 |
| 129 | 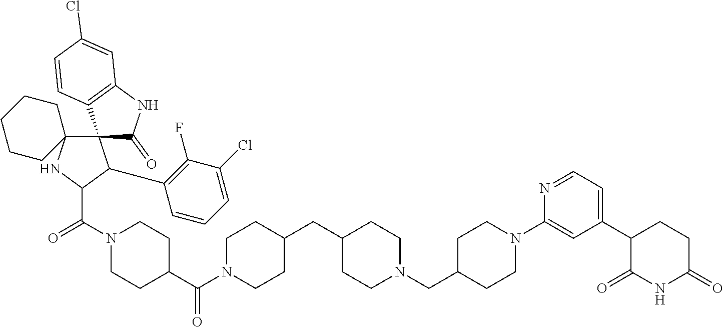 [SMILES: O=C1CCC(c2ccnc(N3CCC(CN4CCC(CC5CCN(C(=O)C6CCN(C(=O)C7NC8(CCCCC8)[C@@]8(C(=O)Nc9cc(Cl)ccc98)C7c7cccc(Cl)c7F)CC6)CC5)CC4)CC3)c2)C(=O)N1] | MDM2 |
| 130 | 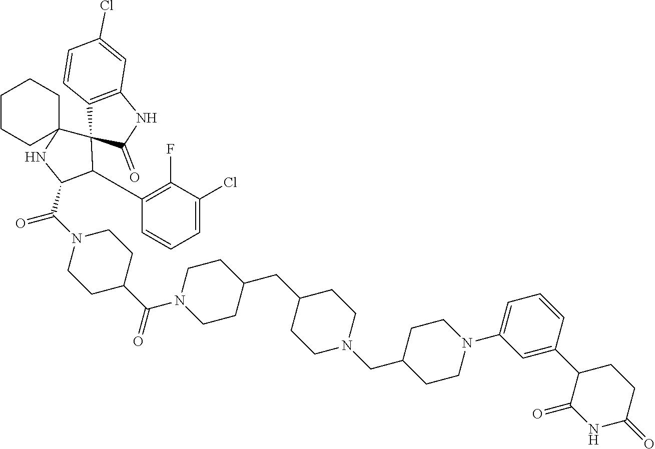 [SMILES: O=C1CCC(c2cccc(N3CCC(CN4CCC(CC5CCN(C(=O)C6CCN(C(=O)[C@@H]7NC8(CCCCC8)[C@@]8(C(=O)Nc9cc(Cl)ccc98)C7c7cccc(Cl)c7F)CC6)CC5)CC4)CC3)c2)C(=O)N1] | MDM2 |
| 131 | 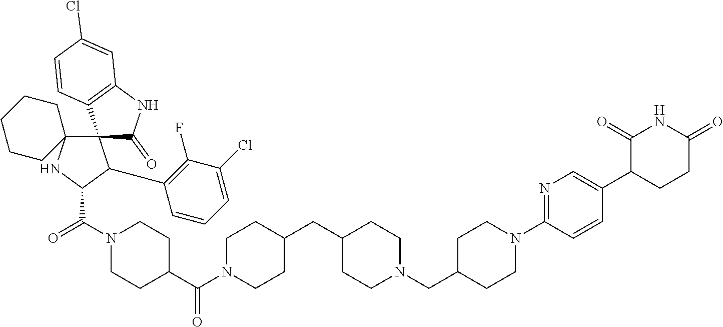 [SMILES: O=C1CCC(c2ccc(N3CCC(CN4CCC(CC5CCN(C(=O)C6CCN(C(=O)[C@@H]7NC8(CCCCC8)[C@@]8(C(=O)Nc9cc(Cl)ccc98)C7c7cccc(Cl)c7F)CC6)CC5)CC4)CC3)nc2)C(=O)N1] | MDM2 |
| 132 | 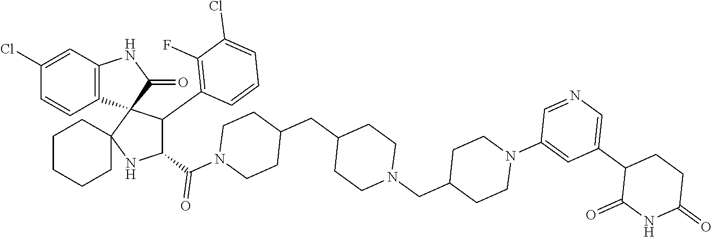 [SMILES: O=C1CCC(c2cncc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)c2)C(=O)N1] | MDM2 |
| 133 | 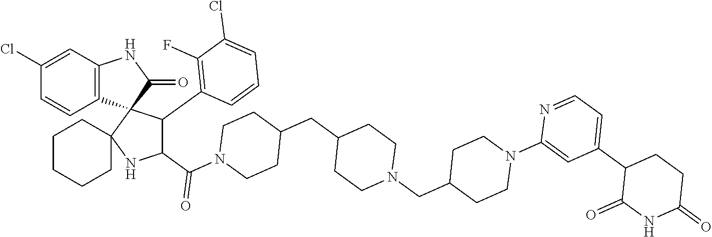 [SMILES: O=C1CCC(c2ccnc(N3CCC(CN4CCC(CC5CCN(C(=O)C6NC7(CCCCC7)[C@@]7(C(=O)Nc8cc(Cl)ccc87)C6c6cccc(Cl)c6F)CC5)CC4)CC3)c2)C(=O)N1] | MDM2 |
| 134 | 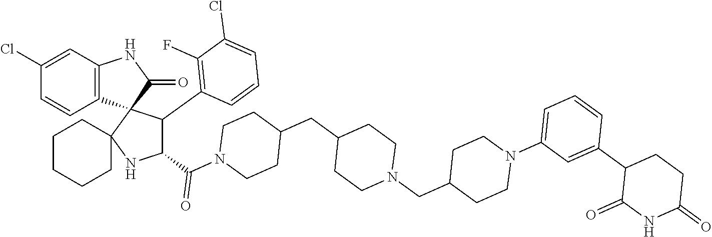 [SMILES: O=C1CCC(c2cccc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)c2)C(=O)N1] | MDM2 |
| 135 | 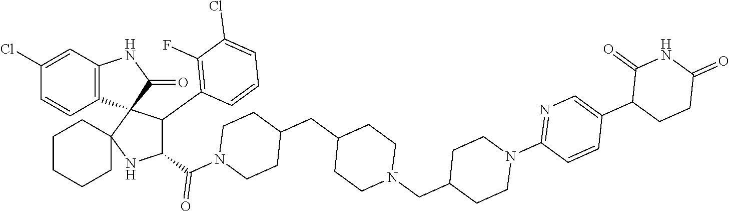 [SMILES: O=C1CCC(c2ccc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)nc2)C(=O)N1] | MDM2 |
| 136 | 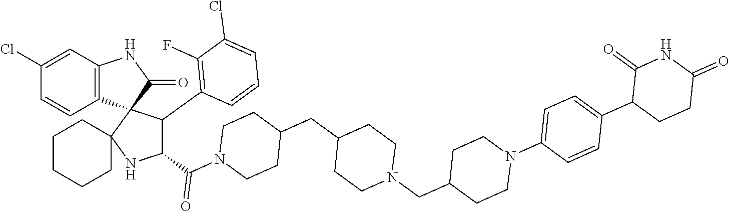 [SMILES: O=C1CCC(c2ccc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)cc2)C(=O)N1] | MDM2 |
| 137 | 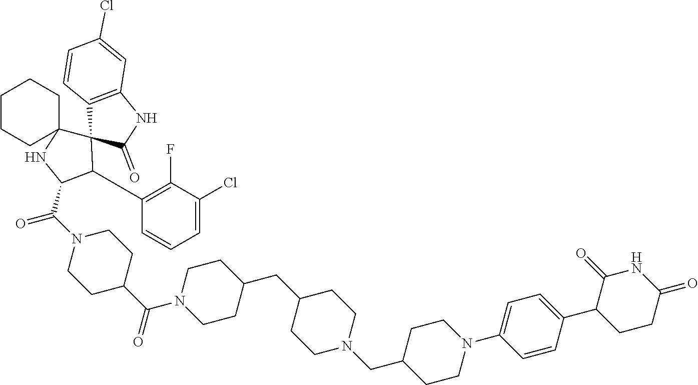 [SMILES: O=C1CCC(c2ccc(N3CCC(CN4CCC(CC5CCN(C(=O)C6CCN(C(=O)[C@@H]7NC8(CCCCC8)[C@@]8(C(=O)Nc9cc(Cl)ccc98)C7c7cccc(Cl)c7F)CC6)CC5)CC4)CC3)cc2)C(=O)N1] | MDM2 |
| 138 | 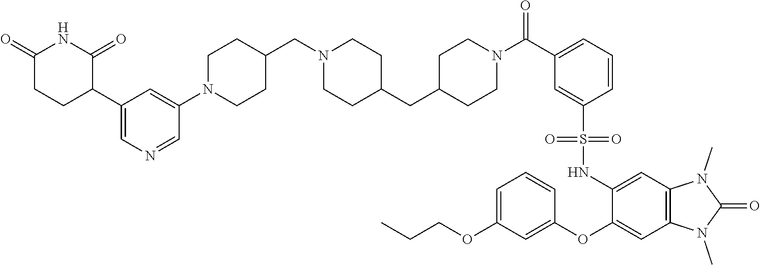 [SMILES: CCCOc1cccc(Oc2cc3c(cc2NS(=O)(=O)c2cccc(C(=O)N4CCC(CC5CCN(CC6CCN(c7cncc(C8CCC(=O)NC8=O)c7)CC6)CC5)CC4)c2)n(C)c(=O)n3C)c1] | TRIM24 |
| 139 | 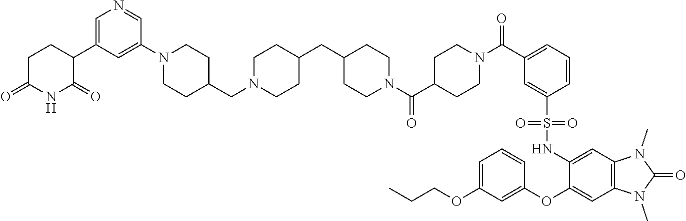 [SMILES: CCCOc1cccc(Oc2cc3c(cc2NS(=O)(=O)c2cccc(C(=O)N4CCC(C(=O)N5CCC(CC6CCN(CC7CCN(c8cncc(C9CCC(=O)NC9=O)c8)CC7)CC6)CC5)CC4)c2)n(C)c(=O)n3C)c1] | TRIM24 |
| 140 | 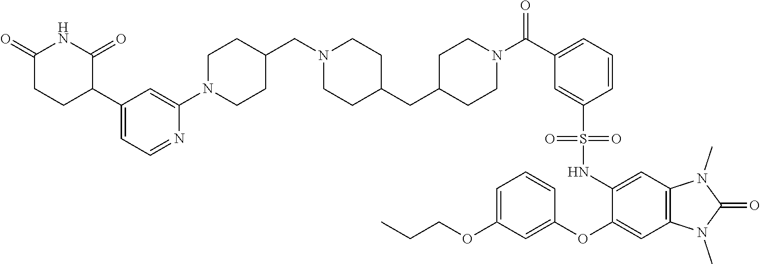 [SMILES: CCCOc1cccc(Oc2cc3c(cc2NS(=O)(=O)c2cccc(C(=O)N4CCC(CC5CCN(CC6CCN(c7cc(C8CCC(=O)NC8=O)ccn7)CC6)CC5)CC4)c2)n(C)c(=O)n3C)c1] | TRIM24 |
| 141 | 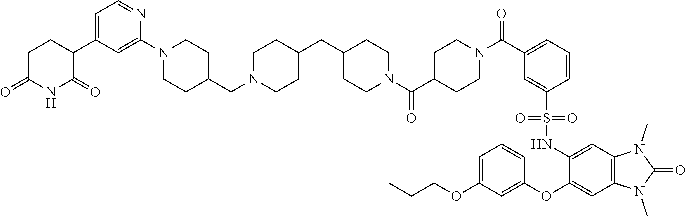 [SMILES: CCCOc1cccc(Oc2cc3c(cc2NS(=O)(=O)c2cccc(C(=O)N4CCC(C(=O)N5CCC(CC6CCN(CC7CCN(c8cc(C9CCC(=O)NC9=O)ccn8)CC7)CC6)CC5)CC4)c2)n(C)c(=O)n3C)c1] | TRIM24 |
| 142 | 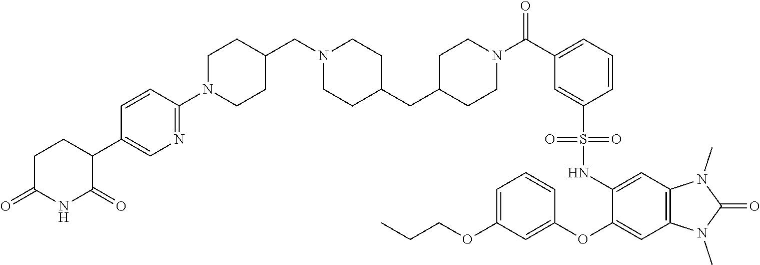 [SMILES: CCCOc1cccc(Oc2cc3c(cc2NS(=O)(=O)c2cccc(C(=O)N4CCC(CC5CCN(CC6CCN(c7ccc(C8CCC(=O)NC8=O)cn7)CC6)CC5)CC4)c2)n(C)c(=O)n3C)c1] | TRIM24 |
| 143 | 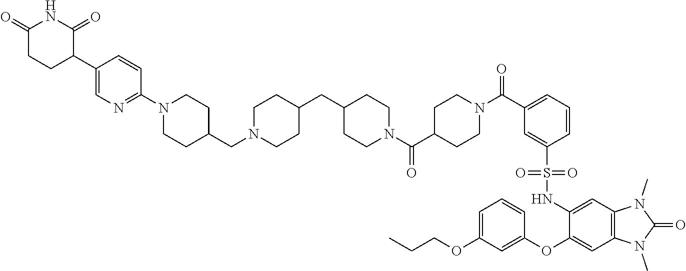 [SMILES: CCCOc1cccc(Oc2cc3c(cc2NS(=O)(=O)c2cccc(C(=O)N4CCC(C(=O)N5CCC(CC6CCN(CC7CCN(c8ccc(C9CCC(=O)NC9=O)cn8)CC7)CC6)CC5)CC4)c2)n(C)c(=O)n3C)c1] | TRIM24 |
| 144 | 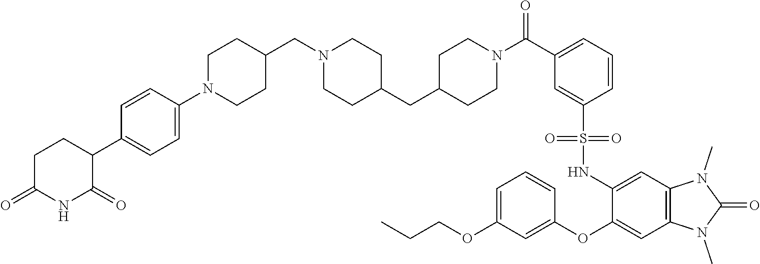 [SMILES: CCCOc1cccc(Oc2cc3c(cc2NS(=O)(=O)c2cccc(C(=O)N4CCC(CC5CCN(CC6CCN(c7ccc(C8CCC(=O)NC8=O)cc7)CC6)CC5)CC4)c2)n(C)c(=O)n3C)c1] | TRIM24 |
| 145 | 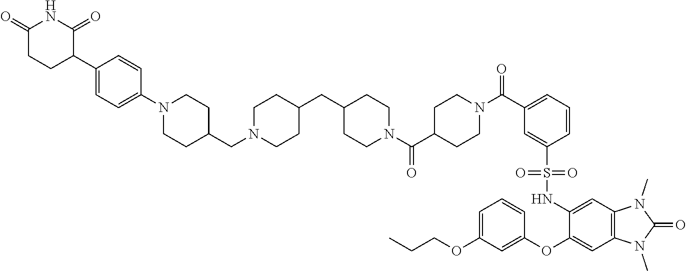 [SMILES: CCCOc1cccc(Oc2cc3c(cc2NS(=O)(=O)c2cccc(C(=O)N4CCC(C(=O)N5CCC(CC6CCN(CC7CCN(c8ccc(C9CCC(=O)NC9=O)cc8)CC7)CC6)CC5)CC4)c2)n(C)c(=O)n3C)c1] | TRIM24 |
| 146 | 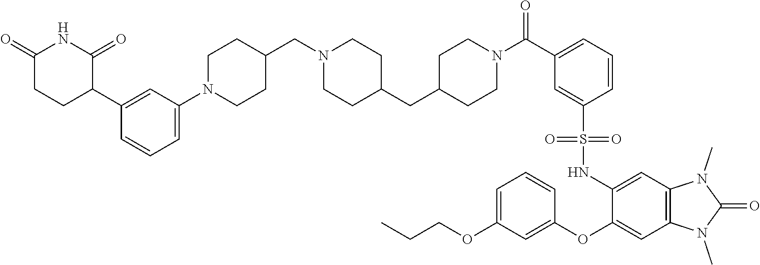 [SMILES: CCCOc1cccc(Oc2cc3c(cc2NS(=O)(=O)c2cccc(C(=O)N4CCC(CC5CCN(CC6CCN(c7cccc(C8CCC(=O)NC8=O)c7)CC6)CC5)CC4)c2)n(C)c(=O)n3C)c1] | TRIM24 |
| 147 | 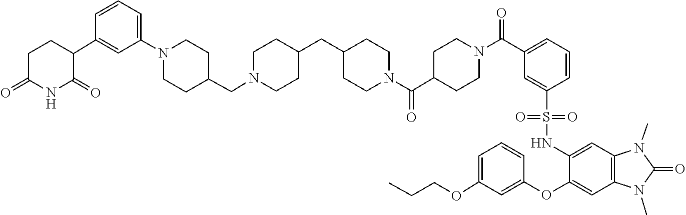 [SMILES: CCCOc1cccc(Oc2cc3c(cc2NS(=O)(=O)c2cccc(C(=O)N4CCC(C(=O)N5CCC(CC6CCN(CC7CCN(c8cccc(C9CCC(=O)NC9=O)c8)CC7)CC6)CC5)CC4)c2)n(C)c(=O)n3C)c1] | TRIM24 |
| 148 | 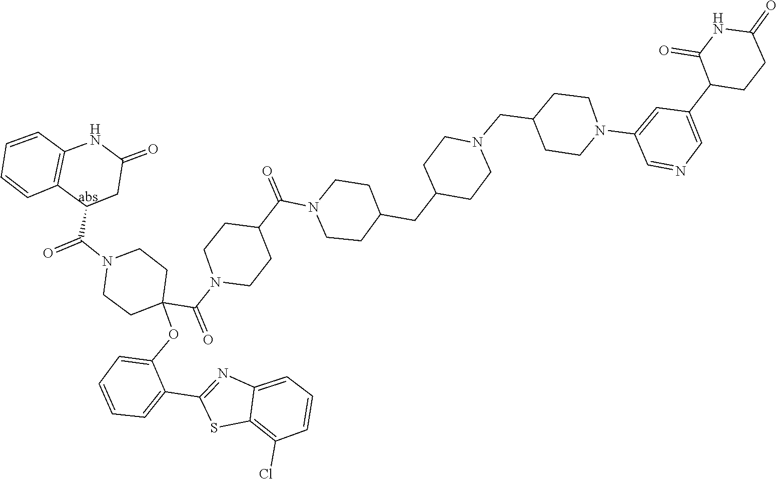 [SMILES: O=C1CCC(c2cncc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc9C8)CC7)CC6)CC5)CC4)CC3)c2)C(=O)N1] | MPro |
| 149 | 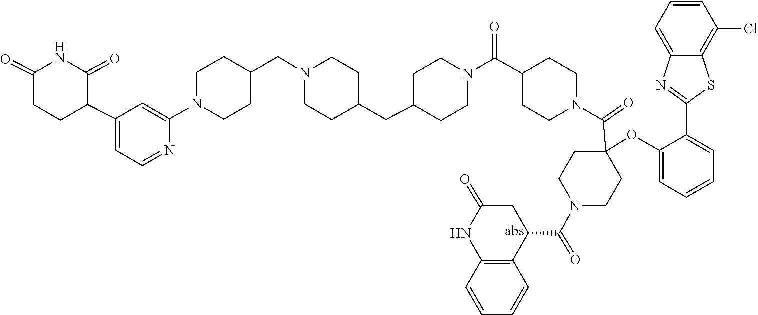 [SMILES: O=C1CCC(c2ccnc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)c2)C(=O)N1] | MPro |
| 150 | 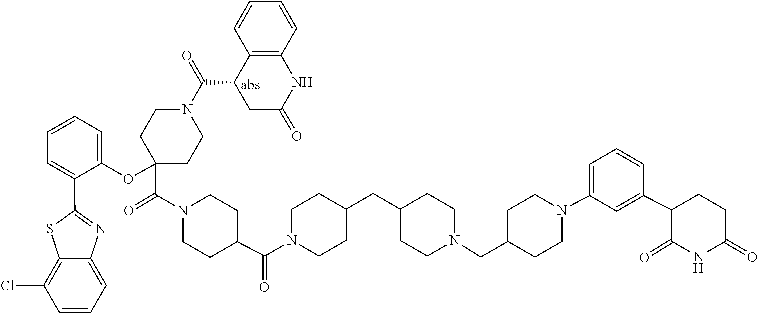 [SMILES: O=C1CCC(c2cccc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)c2)C(=O)N1] | MPro |
| 151 | 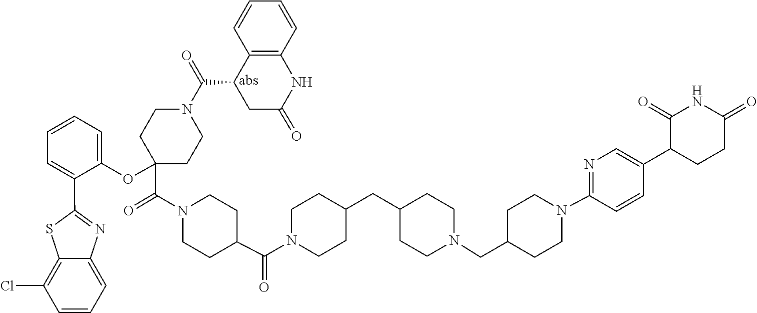 [SMILES: O=C1CCC(c2ccc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)nc2)C(=O)N1] | MPro |
| 152 | 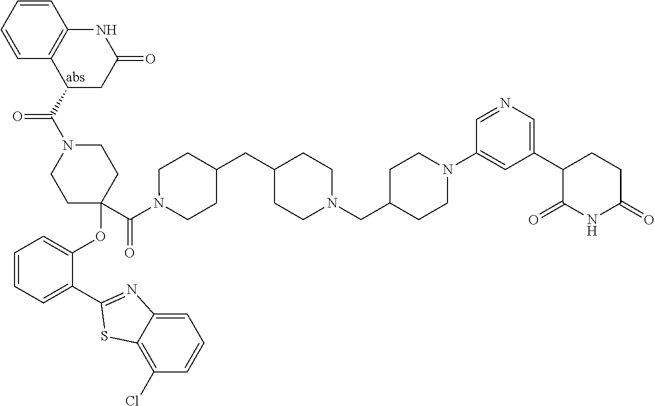 [SMILES: O=C1CCC(c2cncc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] | MPro |
| 153 | 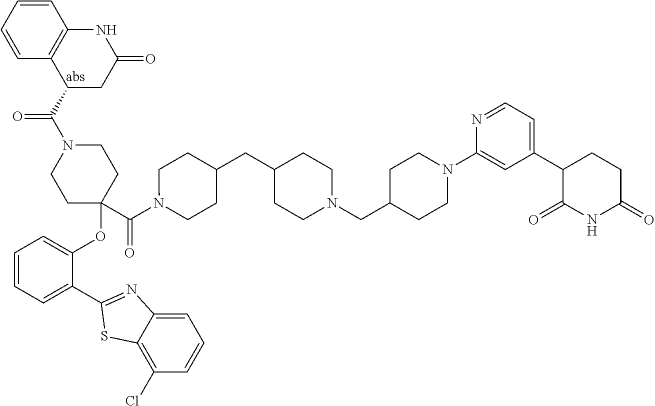 [SMILES: O=C1CCC(c2ccnc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] | MPro |
| 154 | 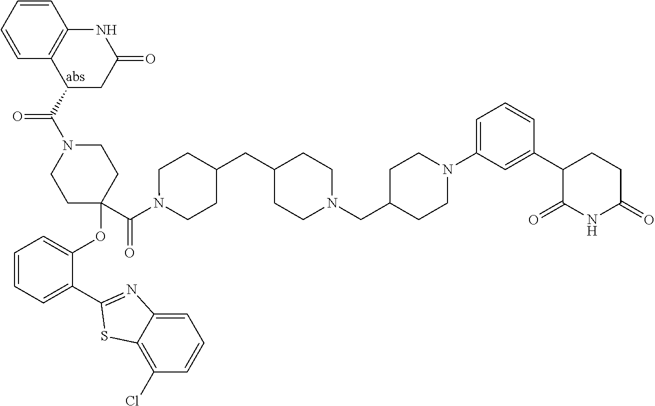 [SMILES: O=C1CCC(c2cccc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] | MPro |
| 155 | 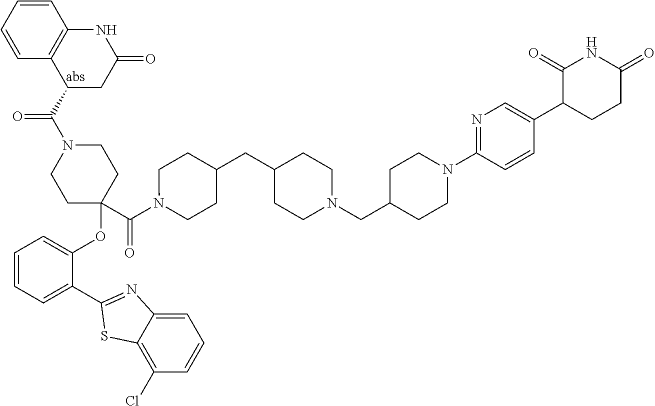 [SMILES: O=C1CCC(c2ccc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)nc2)C(=O)N1] | MPro |
| 156 | 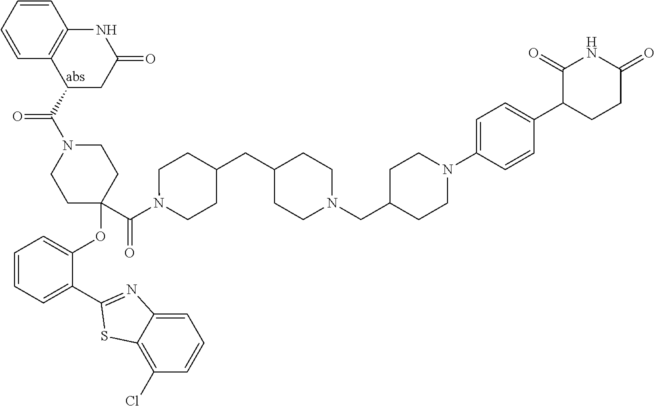 [SMILES: O=C1CCC(c2ccc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)cc2)C(=O)N1] | MPro |
| 157 | 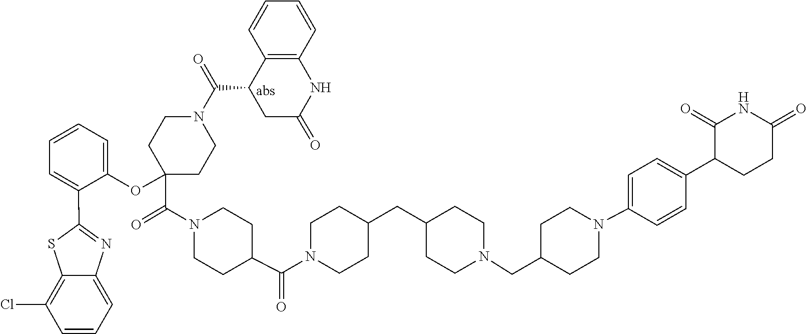 [SMILES: O=C1CCC(c2ccc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)cc2)C(=O)N1] | MPro |
| 158 | 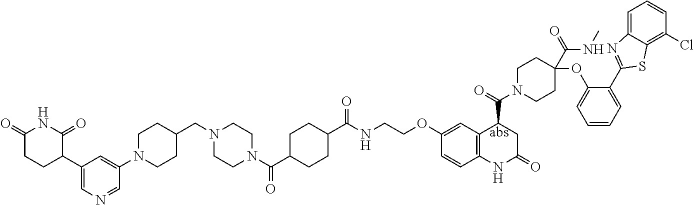 [SMILES: CNC(=O)C1(Oc2ccccc2-c2nc3cccc(Cl)c3s2)CCN(C(=O)[C@H]2CC(=O)Nc3ccc(OCCNC(=O)C4CCC(C(=O)N5CCN(CC6CCN(c7cncc(C8CCC(=O)NC8=O)c7)CC6)CC5)CC4)cc32)CC1] | MPro |
| 159 | 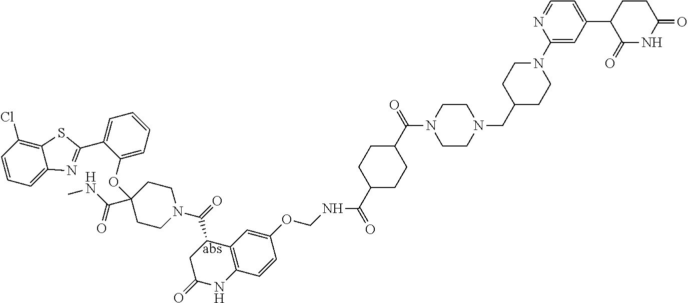 [SMILES: CNC(=O)C1(Oc2ccccc2-c2nc3cccc(Cl)c3s2)CCN(C(=O)[C@H]2CC(=O)Nc3ccc(OCNC(=O)C4CCC(C(=O)N5CCN(CC6CCN(c7cc(C8CCC(=O)NC8=O)ccn7)CC6)CC5)CC4)cc32)CC1] | MPro |
| 160 | 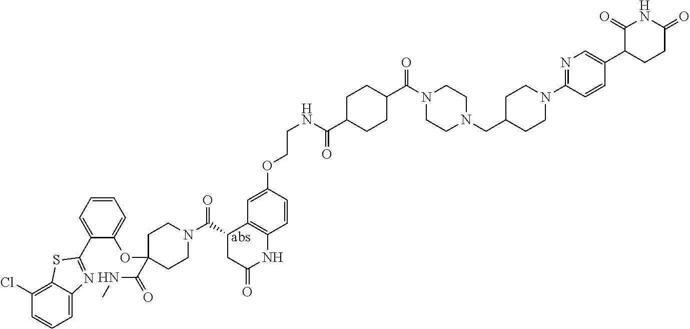 [SMILES: CNC(=O)C1(Oc2ccccc2-c2nc3cccc(Cl)c3s2)CCN(C(=O)[C@H]2CC(=O)Nc3ccc(OCCNC(=O)C4CCC(C(=O)N5CCN(CC6CCN(c7ccc(C8CCC(=O)NC8=O)cn7)CC6)CC5)CC4)cc32)CC1] | MPro |
| 161 | 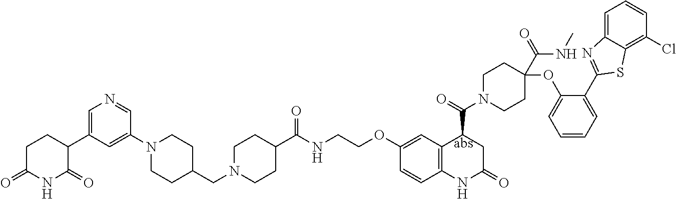 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6cncc(C7CCC(=O)NC7=O)c6)CC5)CC4)cc32)CC1] | MPro |
| 162 | 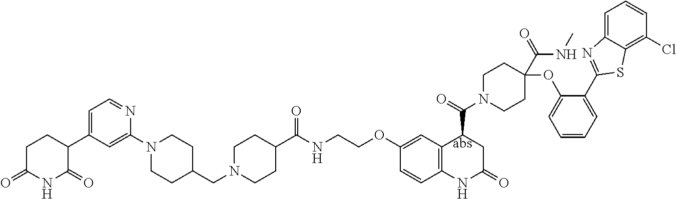 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6cc(C7CCC(=O)NC7=O)ccn6)CC5)CC4)cc32)CC1] | MPro |
| 163 | 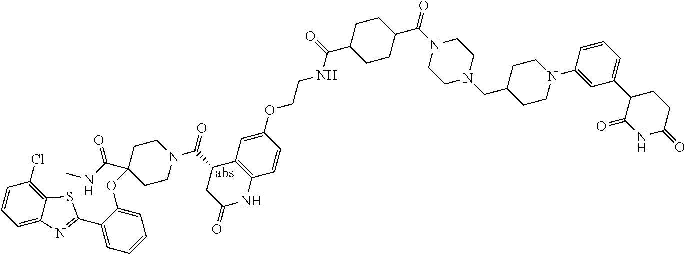 [SMILES: CNC(=O)C1(Oc2ccccc2-c2nc3cccc(Cl)c3s2)CCN(C(=O)[C@H]2CC(=O)Nc3ccc(OCCNC(=O)C4CCC(C(=O)N5CCN(CC6CCN(c7cccc(C8CCC(=O)NC8=O)c7)CC6)CC5)CC4)cc32)CC1] | MPro |
| 164 | 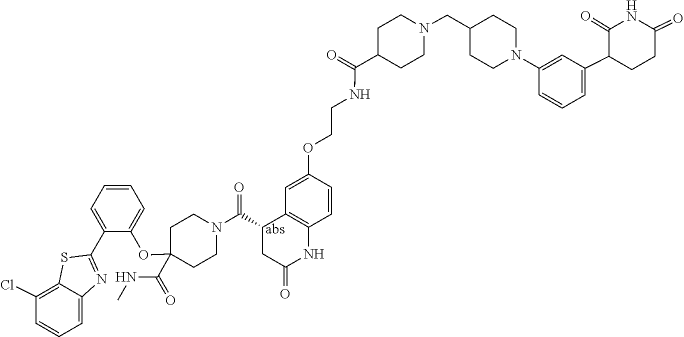 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6cccc(C7CCC(=O)NC7=O)c6)CC5)CC4)cc32)CC1] | MPro |
| 165 | 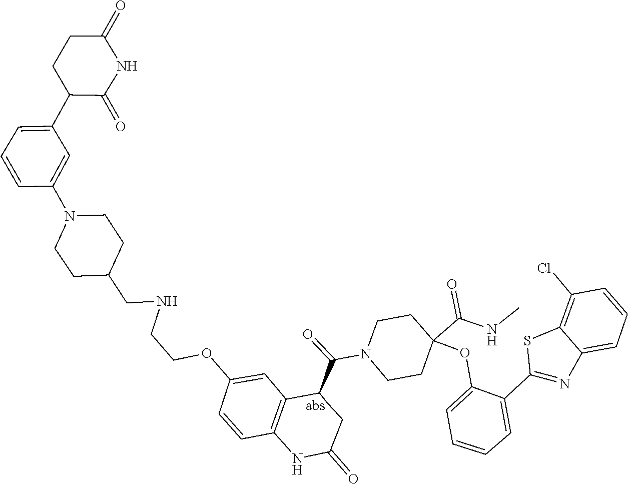 | MPro |
| 166 | 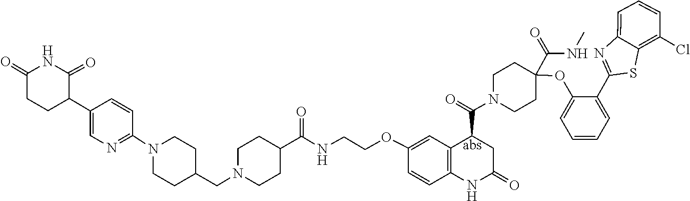 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6ccc(C7CCC(=O)NC7=O)cn6)CC5)CC4)cc32)CC1] | MPro |
| 167 | 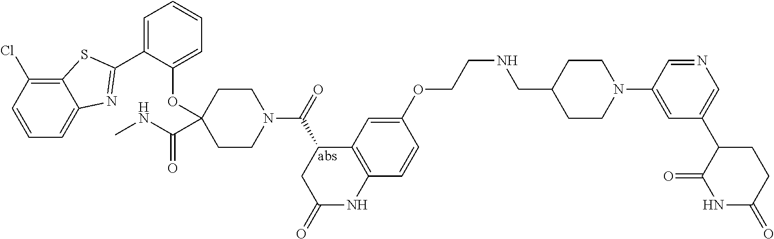 [SMILES: CNC(=O)C1(Oc2ccccc2-c2nc3cccc(Cl)c3s2)CCN(C(=O)[C@H]2CC(=O)Nc3ccc(OCCNCC4CCN(c5cncc(C6CCC(=O)NC6=O)c5)CC4)cc32)CC1] | MPro |
| 168 | 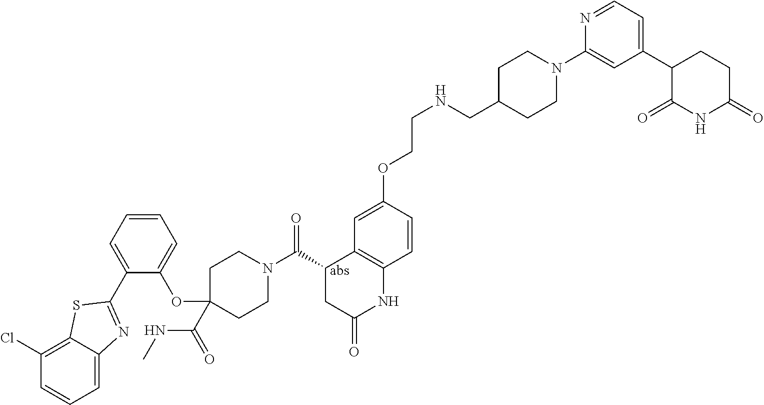 [SMILES: CNC(=O)C1(Oc2ccccc2-c2nc3cccc(Cl)c3s2)CCN(C(=O)[C@H]2CC(=O)Nc3ccc(OCCNCC4CCN(c5cc(C6CCC(=O)NC6=O)ccn5)CC4)cc32)CC1] | MPro |
| 169 | 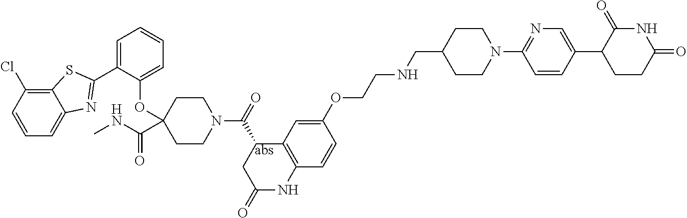 [SMILES: CNC(=O)C1(Oc2ccccc2-c2nc3cccc(Cl)c3s2)CCN(C(=O)[C@H]2CC(=O)Nc3ccc(OCCNCC4CCN(c5ccc(C6CCC(=O)NC6=O)cn5)CC4)cc32)CC1] | MPro |
| 170 | 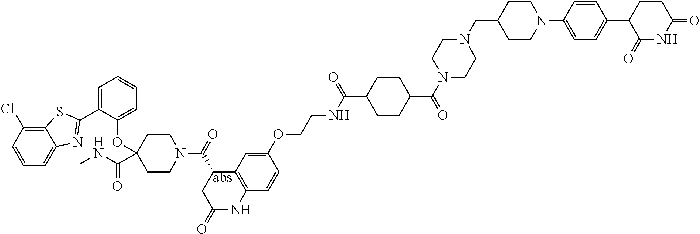 [SMILES: CNC(=O)C1(Oc2ccccc2-c2nc3cccc(Cl)c3s2)CCN(C(=O)[C@H]2CC(=O)Nc3ccc(OCCNC(=O)C4CCC(C(=O)N5CCN(CC6CCN(c7ccc(C8CCC(=O)NC8=O)cc7)CC6)CC5)CC4)cc32)CC1] | MPro |
| 171 | 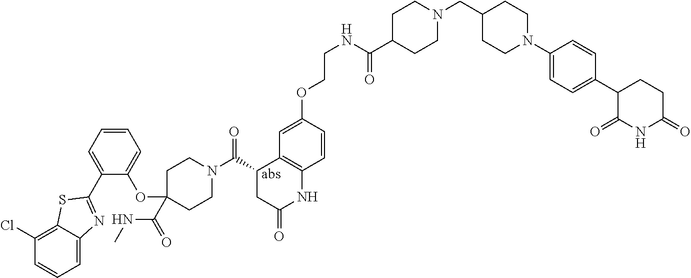 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6ccc(C7CCC(=O)NC7=O)cc6)CC5)CC4)cc32)CC1] | MPro |
| 172 | 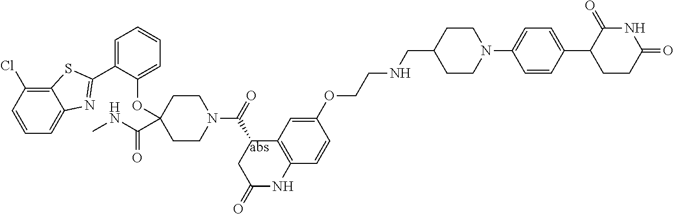 [SMILES: CNC(=O)C1(Oc2ccccc2-c2nc3cccc(Cl)c3s2)CCN(C(=O)[C@H]2CC(=O)Nc3ccc(OCCNCC4CCN(c5ccc(C6CCC(=O)NC6=O)cc5)CC4)cc32)CC1] | MPro |
| 173 | 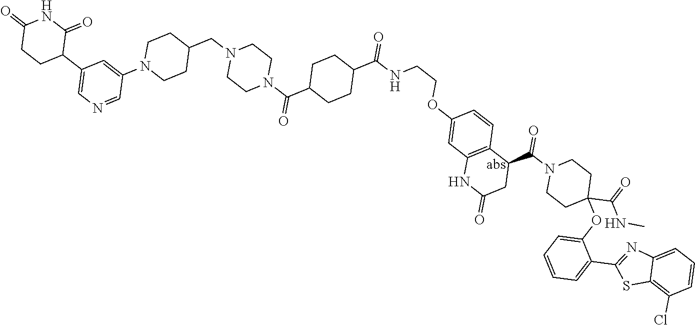 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7cncc(C8CCC(=O)NC8=O)c7)CC6)CC5)CC4)ccc32)CC1] | MPro |
| 174 | 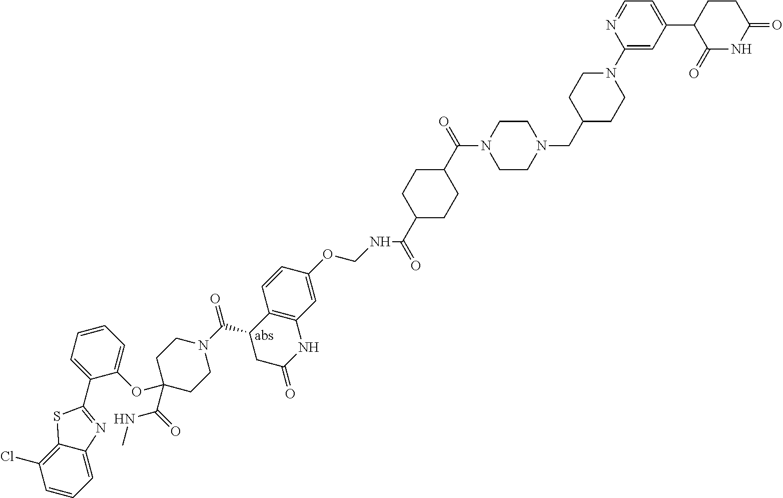 [SMILES: CNC(=O)C1(Oc2ccccc2-c2nc3cccc(Cl)c3s2)CCN(C(=O)[C@H]2CC(=O)Nc3cc(OCNC(=O)C4CCC(C(=O)N5CCN(CC6CCN(c7cc(C8CCC(=O)NC8=O)ccn7)CC6)CC5)CC4)ccc32)CC1] | MPro |
| 175 | 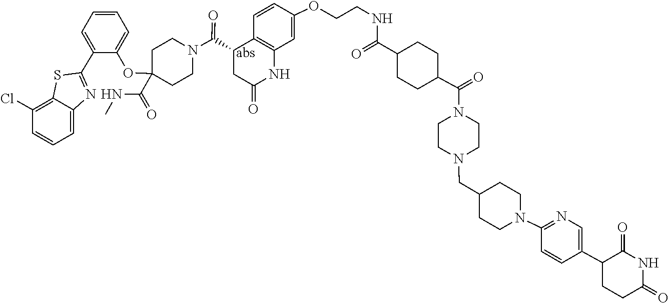 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7ccc(C8CCC(=O)NC8=O)cn7)CC6)CC5)CC4)ccc32)CC1] | MPro |
| 176 | 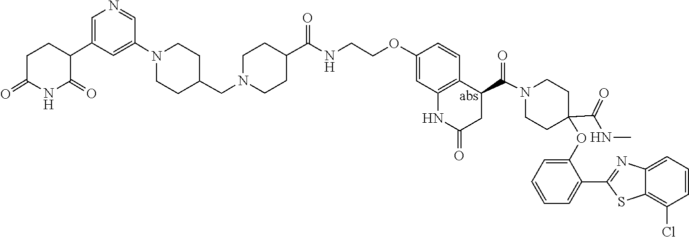 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6cncc(C7CCC(=O)NC7=O)c6)CC5)CC4)ccc32)CC1] | MPro |
| 177 | 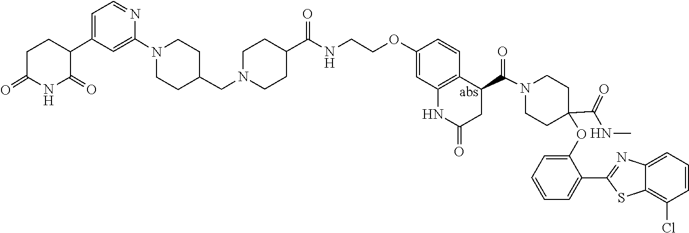 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6cc(C7CCC(=O)NC7=O)ccn6)CC5)CC4)ccc32)CC1] | MPro |
| 178 | 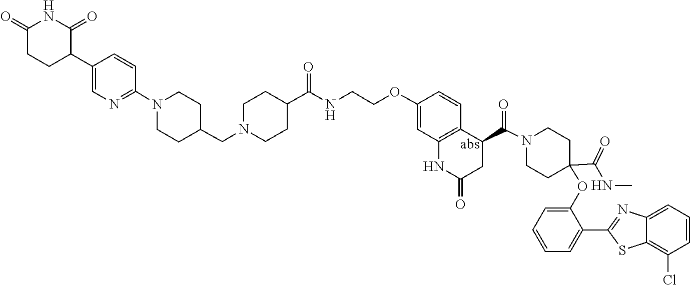 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6ccc(C7CCC(=O)NC7=O)cn6)CC5)CC4)ccc32)CC1] | MPro |
| 179 | 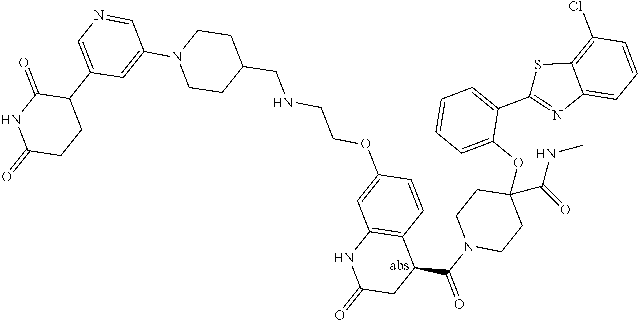 [SMILES: CNC(=O)C1(Oc2ccccc2-c2nc3cccc(Cl)c3s2)CCN(C(=O)[C@H]2CC(=O)Nc3cc(OCCNCC4CCN(c5cncc(C6CCC(=O)NC6=O)c5)CC4)ccc32)CC1] | MPro |
| 180 | 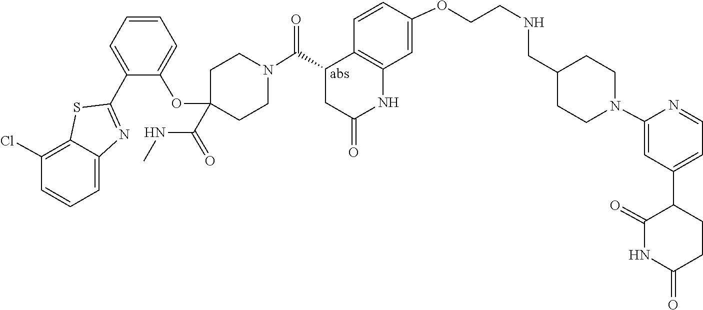 [SMILES: CNC(=O)C1(Oc2ccccc2-c2nc3cccc(Cl)c3s2)CCN(C(=O)[C@H]2CC(=O)Nc3cc(OCCNCC4CCN(c5cc(C6CCC(=O)NC6=O)ccn5)CC4)ccc32)CC1] | MPro |
| 181 | 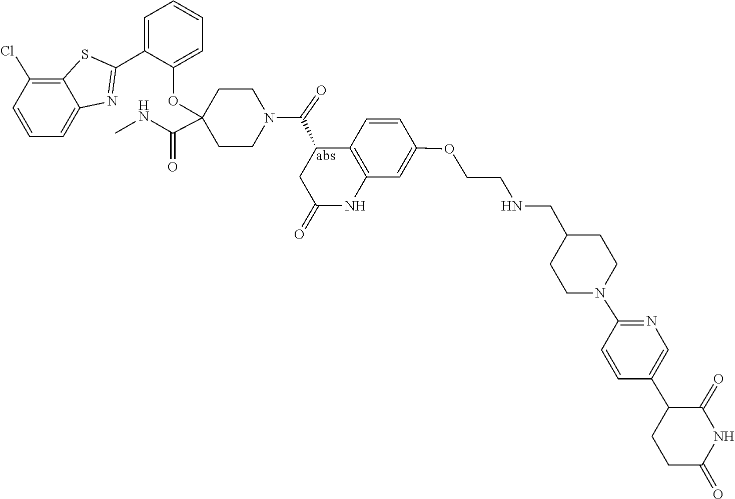 [SMILES: CNC(=O)C1(Oc2ccccc2-c2nc3cccc(Cl)c3s2)CCN(C(=O)[C@H]2CC(=O)Nc3cc(OCCNCC4CCN(c5ccc(C6CCC(=O)NC6=O)cn5)CC4)ccc32)CC1] | MPro |
| 182 | 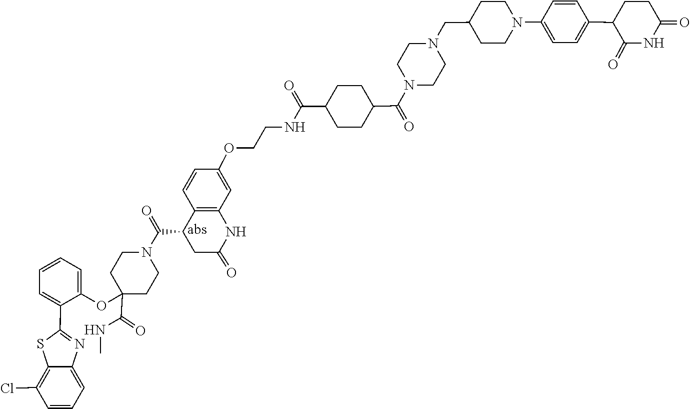 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7ccc(C8CCC(=O)NC8=O)cc7)CC6)CC5)CC4)ccc32)CC1] | MPro |
| 183 | 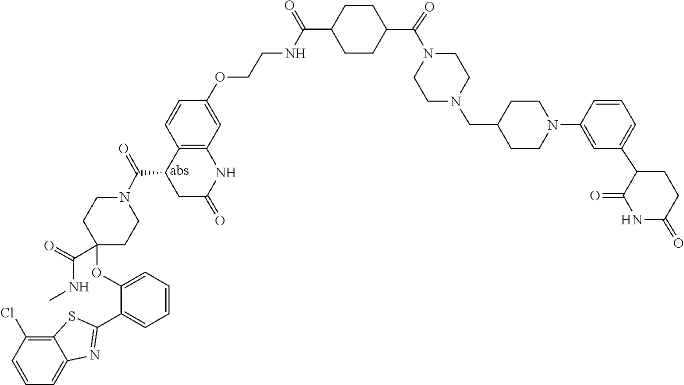 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7cccc(C8CCC(=O)NC8=O)c7)CC6)CC5)CC4)ccc32)CC1] | MPro |
| 184 | 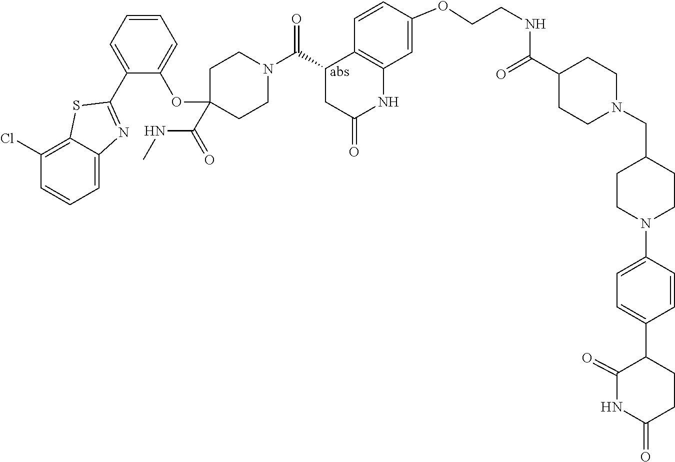 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6ccc(C7CCC(=O)NC7=O)cc6)CC5)CC4)ccc32)CC1] | MPro |
| 185 | 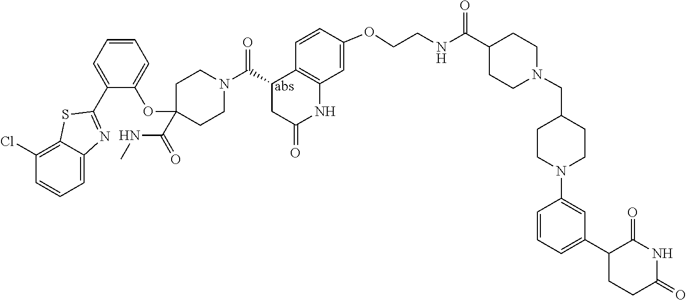 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6cccc(C7CCC(=O)NC7=O)c6)CC5)CC4)ccc32)CC1] | MPro |
| 186 | 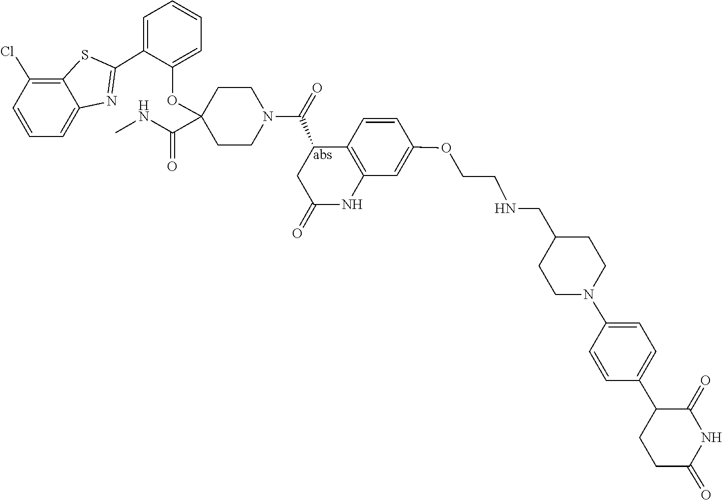 [SMILES: CNC(=O)C1(Oc2ccccc2-c2nc3cccc(Cl)c3s2)CCN(C(=O)[C@H]2CC(=O)Nc3cc(OCCNCC4CCN(c5ccc(C6CCC(=O)NC6=O)cc5)CC4)ccc32)CC1] | MPro |
| 187 | 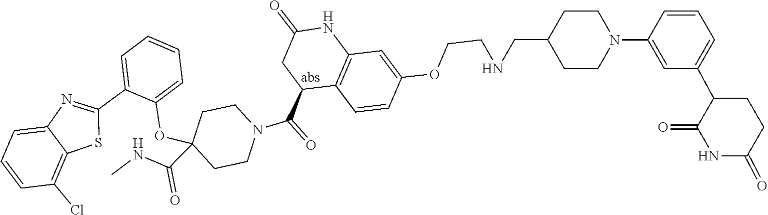 [SMILES: CNC(=O)C1(Oc2ccccc2-c2nc3cccc(Cl)c3s2)CCN(C(=O)[C@H]2CC(=O)Nc3cc(OCCNCC4CCN(c5cccc(C6CCC(=O)NC6=O)c5)CC4)ccc32)CC1] | MPro |
| 188 | 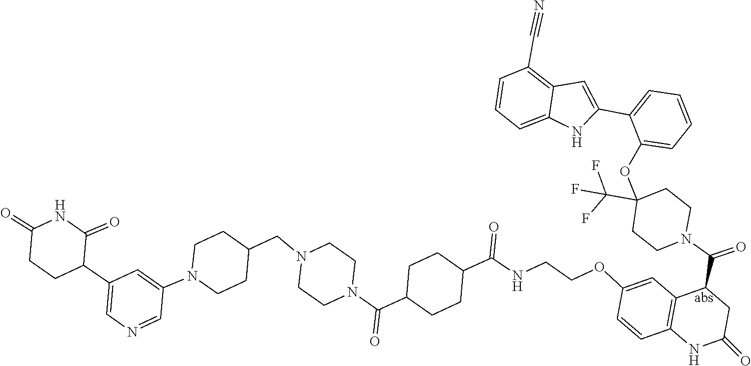 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cncc(C%10CCC(=O)NC%10=O)c9)CC8)CC7)CC6)cc54)CC3)cc12] | MPro |
| 189 | 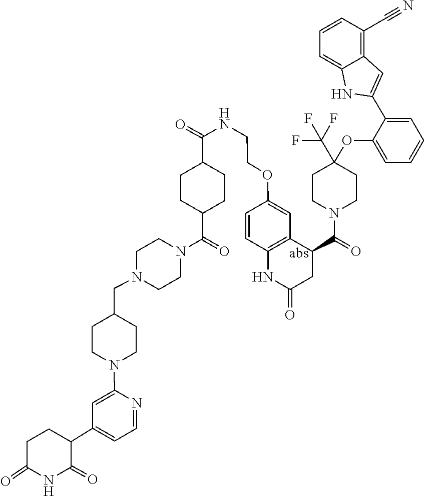 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cc(C%10CCC(=O)NC%10=O)ccn9)CC8)CC7)CC6)cc54)CC3)cc12] | MPro |
| 190 | 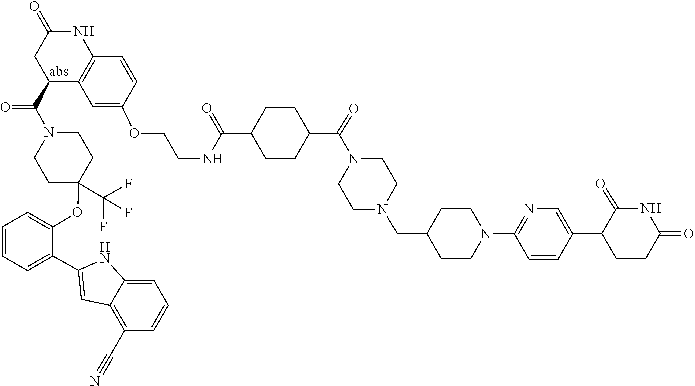 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cn9)CC8)CC7)CC6)cc54)CC3)cc12] | MPro |
| 191 | 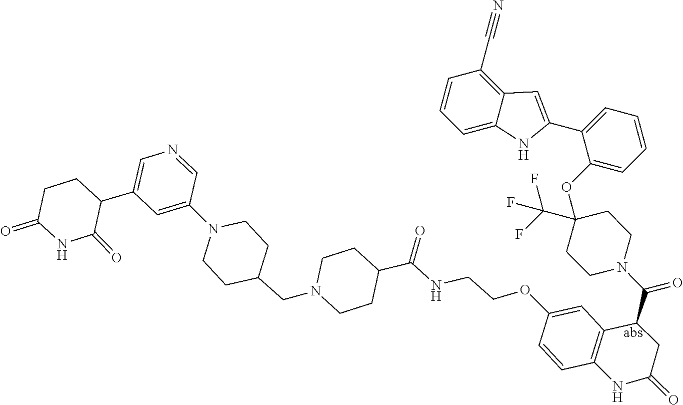 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cncc(C9CCC(=O)NC9=O)c8)CC7)CC6)cc54)CC3)cc12] | MPro |
| 192 | 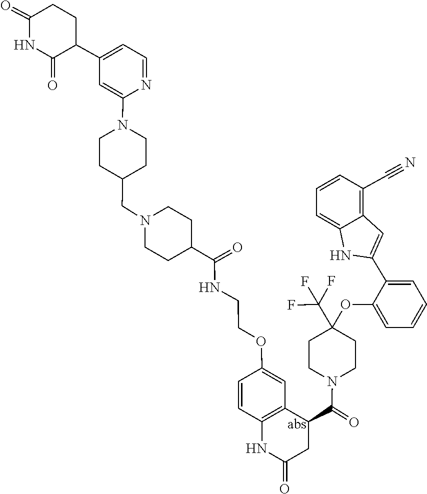 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cc(C9CCC(=O)NC9=O)ccn8)CC7)CC6)cc54)CC3)cc12] | MPro |
| 193 | 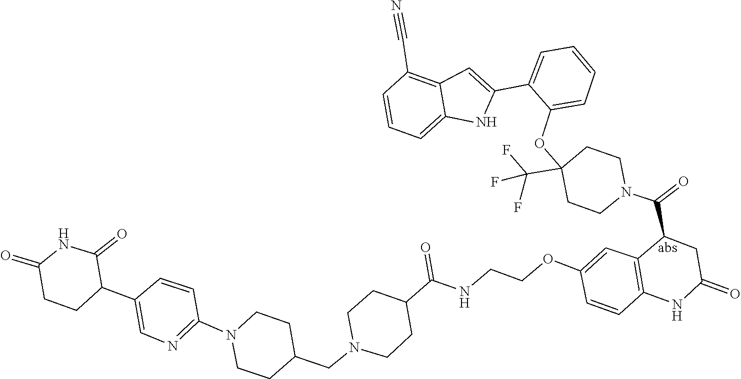 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cn8)CC7)CC6)cc54)CC3)cc12] | MPro |
| 194 | 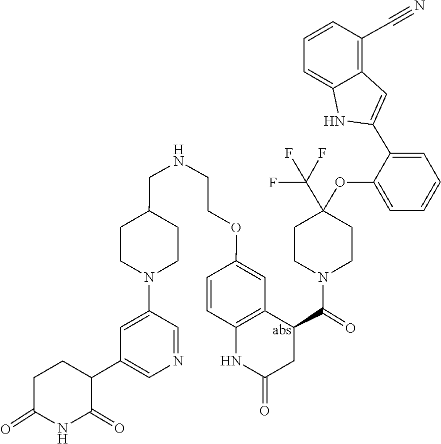 [SMILES: N#Cc1cccc2[nH]c(-c3ccccc3OC3(C(F)(F)F)CCN(C(=O)[C@H]4CC(=O)Nc5ccc(OCCNCC6CCN(c7cncc(C8CCC(=O)NC8=O)c7)CC6)cc54)CC3)cc12] | MPro |
| 195 | 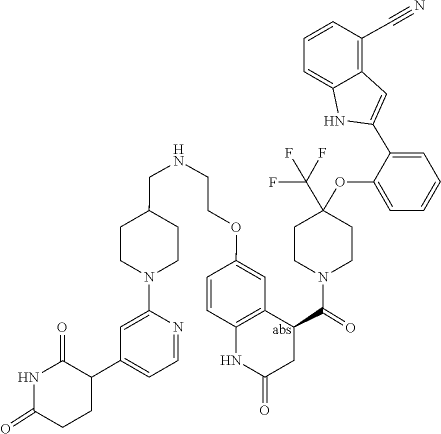 [SMILES: N#Cc1cccc2[nH]c(-c3ccccc3OC3(C(F)(F)F)CCN(C(=O)[C@H]4CC(=O)Nc5ccc(OCCNCC6CCN(c7cc(C8CCC(=O)NC8=O)ccn7)CC6)cc54)CC3)cc12] | MPro |
| 196 | 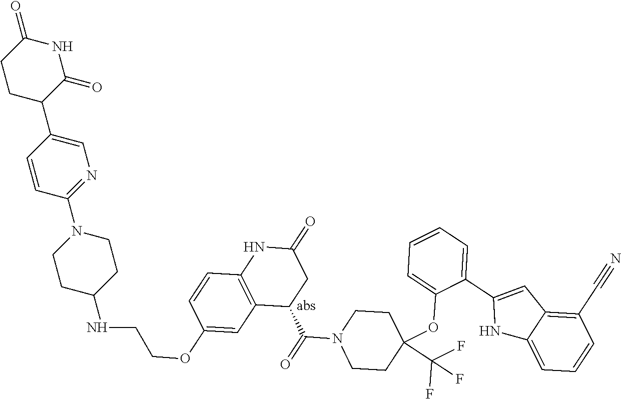 [SMILES: N#Cc1cccc2[nH]c(-c3ccccc3OC3(C(F)(F)F)CCN(C(=O)[C@H]4CC(=O)Nc5ccc(OCCNC6CCN(c7ccc(C8CCC(=O)NC8=O)cn7)CC6)cc54)CC3)cc12] | MPro |
| 197 | 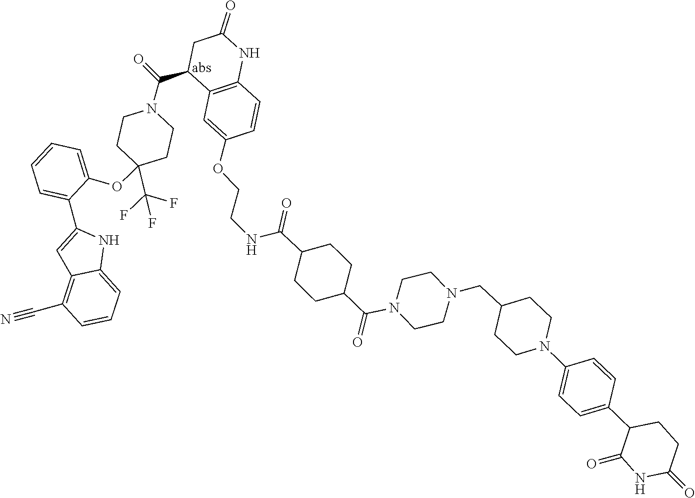 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cc9)CC8)CC7)CC6)cc54)CC3)cc12] | MPro |
| 198 | 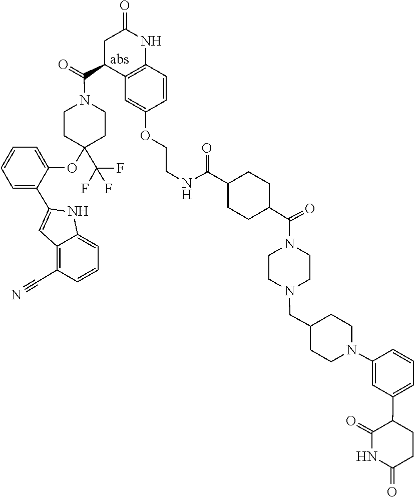 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cccc(C%10CCC(=O)NC%10=O)c9)CC8)CC7)CC6)cc54)CC3)cc12] | MPro |
| 199 | 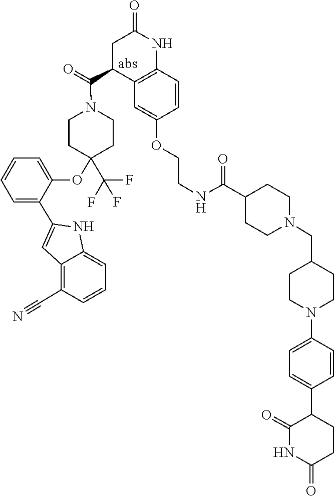 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cc8)CC7)CC6)cc54)CC3)cc12] | MPro |
| 200 | 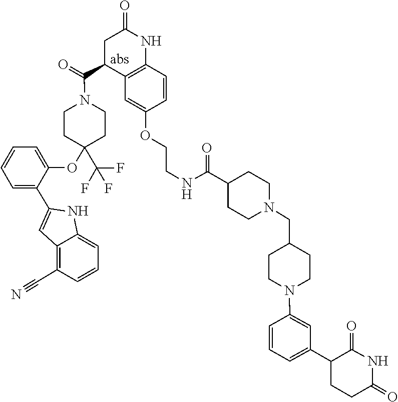 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cccc(C9CCC(=O)NC9=O)c8)CC7)CC6)cc54)CC3)cc12] | MPro |
| 201 | 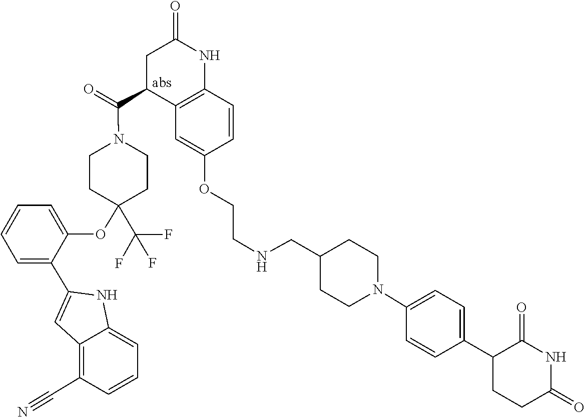 [SMILES: N#Cc1cccc2[nH]c(-c3ccccc3OC3(C(F)(F)F)CCN(C(=O)[C@H]4CC(=O)Nc5ccc(OCCNCC6CCN(c7ccc(C8CCC(=O)NC8=O)cc7)CC6)cc54)CC3)cc12] | MPro |
| 202 | 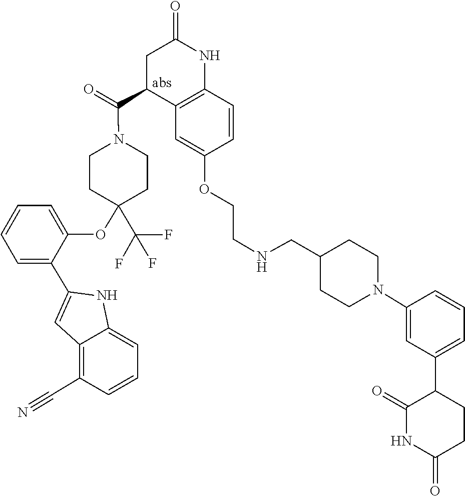 [SMILES: N#Cc1cccc2[nH]c(-c3ccccc3OC3(C(F)(F)F)CCN(C(=O)[C@H]4CC(=O)Nc5ccc(OCCNCC6CCN(c7cccc(C8CCC(=O)NC8=O)c7)CC6)cc54)CC3)cc12] | MPro |
| 203 | 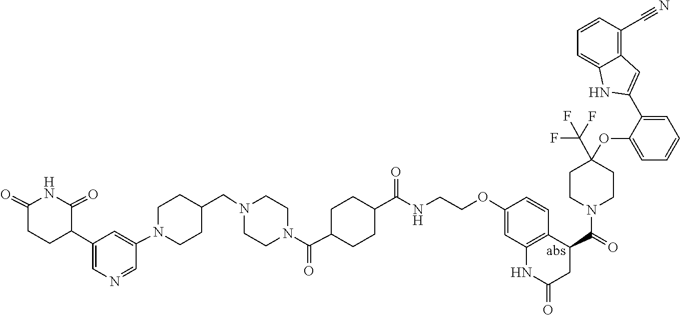 [SMILES: N#Cc1cccc2[nH]c(-c3ccccc3OC3(C(F)(F)F)CCN(C(=O)[C@H]4CC(=O)Nc5cc(OCCNC(=O)C6CCC(C(=O)N7CCN(CC8CCN(c9cncc(C%10CCC(=O)NC%10=O)c9)CC8)CC7)CC6)ccc54)CC3)cc12] | MPro |
| 204 | 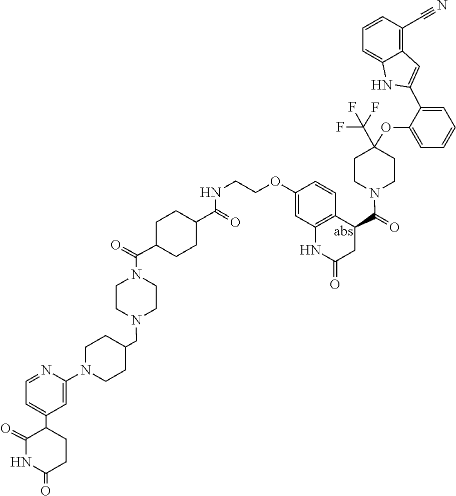 [SMILES: N#Cc1cccc2[nH]c(-c3ccccc3OC3(C(F)(F)F)CCN(C(=O)[C@H]4CC(=O)Nc5cc(OCCNC(=O)C6CCC(C(=O)N7CCN(CC8CCN(c9cc(C%10CCC(=O)NC%10=O)ccn9)CC8)CC7)CC6)ccc54)CC3)cc12] | MPro |
| 205 | 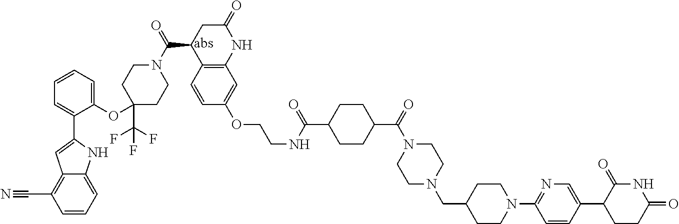 [SMILES: N#Cc1cccc2[nH]c(-c3ccccc3OC3(C(F)(F)F)CCN(C(=O)[C@H]4CC(=O)Nc5cc(OCCNC(=O)C6CCC(C(=O)N7CCN(CC8CCN(c9ccc(C%10CCC(=O)NC%10=O)cn9)CC8)CC7)CC6)ccc54)CC3)cc12] | MPro |
| 206 | 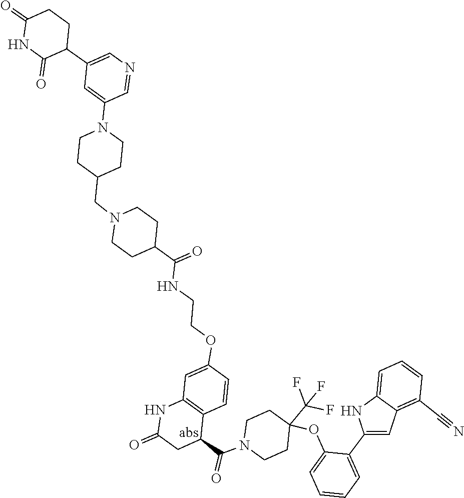 [SMILES: N#Cc1cccc2[nH]c(-c3ccccc3OC3(C(F)(F)F)CCN(C(=O)[C@H]4CC(=O)Nc5cc(OCCNC(=O)C6CCN(CC7CCN(c8cncc(C9CCC(=O)NC9=O)c8)CC7)CC6)ccc54)CC3)cc12] | MPro |
| 207 | 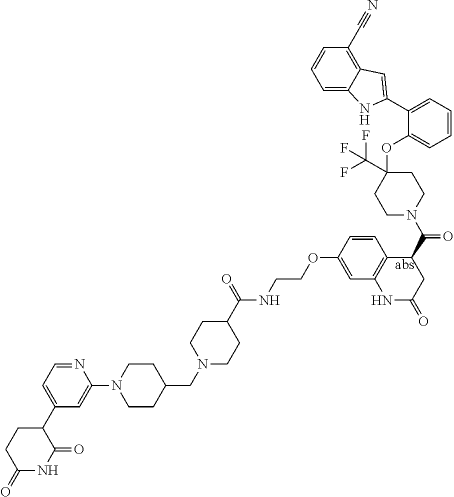 [SMILES: N#Cc1cccc2[nH]c(-c3ccccc3OC3(C(F)(F)F)CCN(C(=O)[C@H]4CC(=O)Nc5cc(OCCNC(=O)C6CCN(CC7CCN(c8cc(C9CCC(=O)NC9=O)ccn8)CC7)CC6)ccc54)CC3)cc12] | MPro |
| 208 | 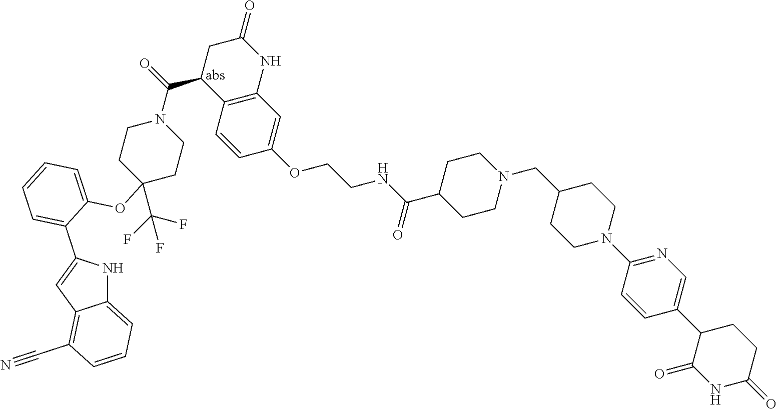 [SMILES: N#Cc1cccc2[nH]c(-c3ccccc3OC3(C(F)(F)F)CCN(C(=O)[C@H]4CC(=O)Nc5cc(OCCNC(=O)C6CCN(CC7CCN(c8ccc(C9CCC(=O)NC9=O)cn8)CC7)CC6)ccc54)CC3)cc12] | MPro |
| 209 | 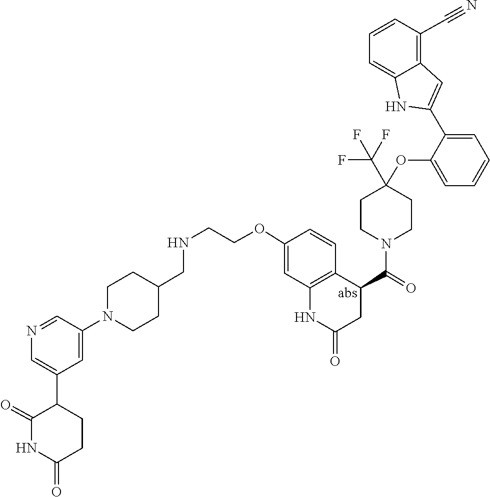 [SMILES: N#Cc1cccc2[nH]c(-c3ccccc3OC3(C(F)(F)F)CCN(C(=O)[C@H]4CC(=O)Nc5cc(OCCNCC6CCN(c7cncc(C8CCC(=O)NC8=O)c7)CC6)ccc54)CC3)cc12] | MPro |
| 210 | 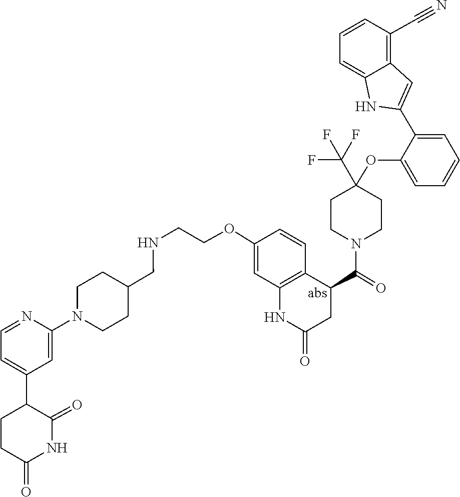 [SMILES: N#Cc1cccc2[nH]c(-c3ccccc3OC3(C(F)(F)F)CCN(C(=O)[C@H]4CC(=O)Nc5cc(OCCNCC6CCN(c7cc(C8CCC(=O)NC8=O)ccn7)CC6)ccc54)CC3)cc12] | MPro |
| 211 | 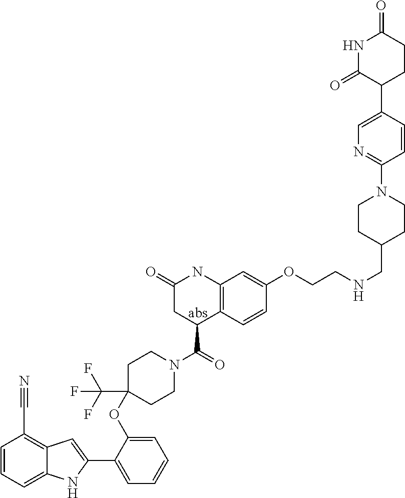 [SMILES: N#Cc1cccc2[nH]c(-c3ccccc3OC3(C(F)(F)F)CCN(C(=O)[C@H]4CC(=O)Nc5cc(OCCNCC6CCN(c7ccc(C8CCC(=O)NC8=O)cn7)CC6)ccc54)CC3)cc12] | MPro |
| 212 | 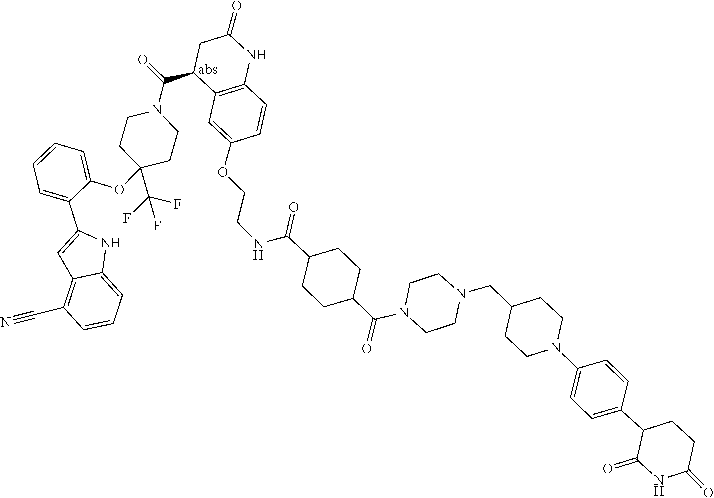 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cc9)CC8)CC7)CC6)cc54)CC3)cc12] | MPro |
| 213 | 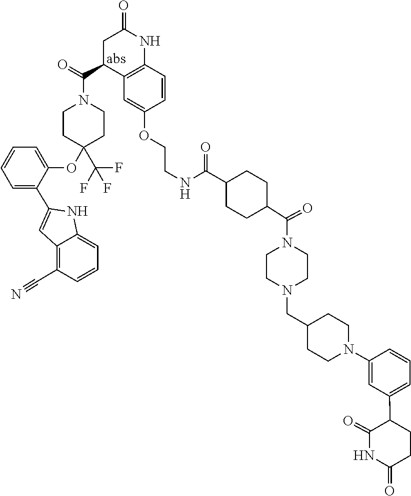 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cccc(C%10CCC(=O)NC%10=O)c9)CC8)CC7)CC6)cc54)CC3)cc12] | MPro |
| 214 | 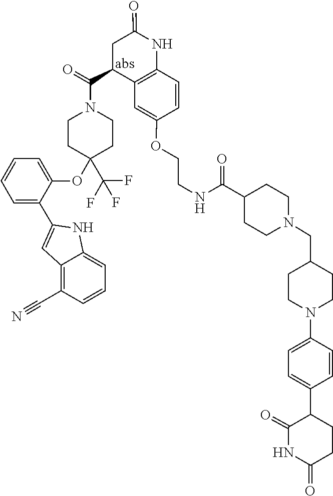 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cc8)CC7)CC6)cc54)CC3)cc12] | MPro |
| 215 | 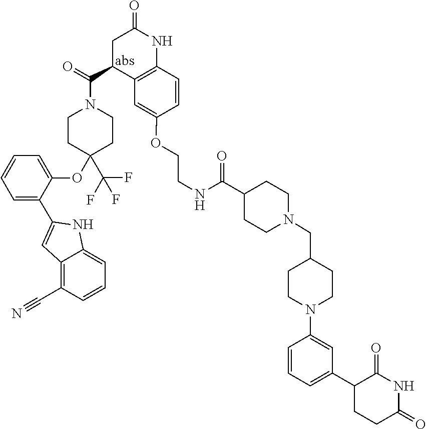 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cccc(C9CCC(=O)NC9=O)c8)CC7)CC6)cc54)CC3)cc12] | MPro |
| 216 | 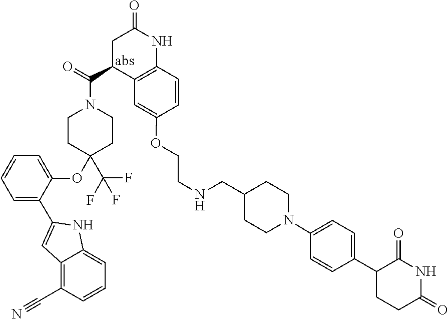 [SMILES: N#Cc1cccc2[nH]c(-c3ccccc3OC3(C(F)(F)F)CCN(C(=O)[C@H]4CC(=O)Nc5ccc(OCCNCC6CCN(c7ccc(C8CCC(=O)NC8=O)cc7)CC6)cc54)CC3)cc12] | MPro |
| 217 | 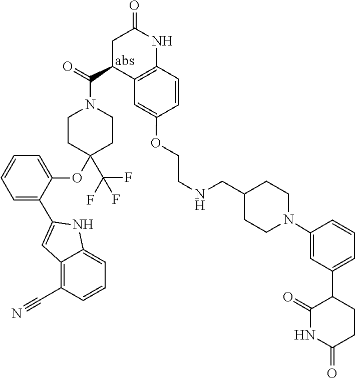 [SMILES: N#Cc1cccc2[nH]c(-c3ccccc3OC3(C(F)(F)F)CCN(C(=O)[C@H]4CC(=O)Nc5ccc(OCCNCC6CCN(c7cccc(C8CCC(=O)NC8=O)c7)CC6)cc54)CC3)cc12] | MPro |
| 218 | 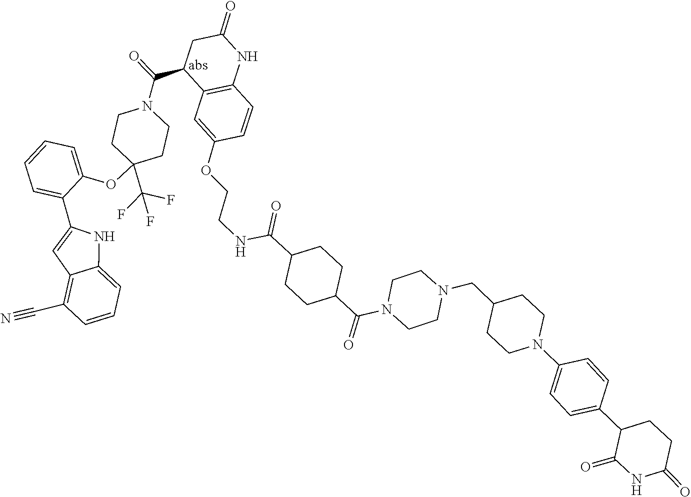 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cc9)CC8)CC7)CC6)cc54)CC3)cc12] | MPro |
| 219 | 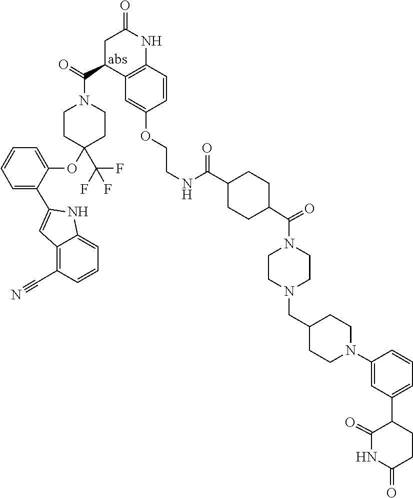 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cccc(C%10CCC(=O)NC%10=O)c9)CC8)CC7)CC6)cc54)CC3)cc12] | MPro |
| 220 | 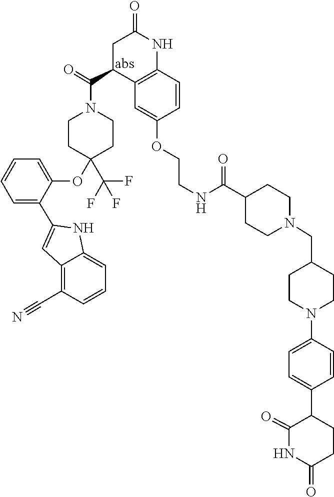 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cc8)CC7)CC6)cc54)CC3)cc12] | MPro |
| 221 | 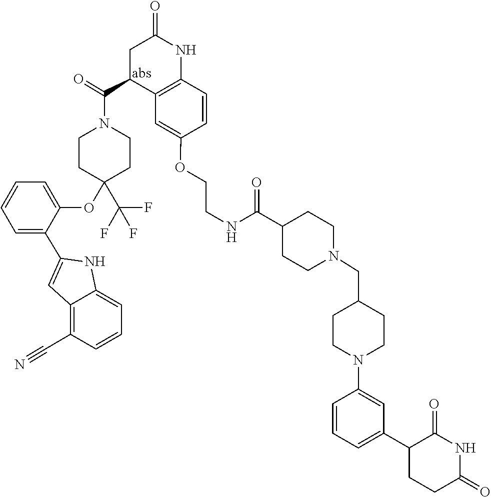 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cccc(C9CCC(=O)NC9=O)c8)CC7)CC6)cc54)CC3)cc12] | MPro |
| 222 | 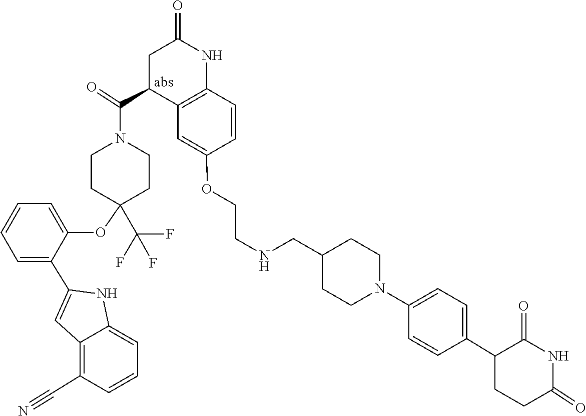 [SMILES: N#Cc1cccc2[nH]c(-c3ccccc3OC3(C(F)(F)F)CCN(C(=O)[C@H]4CC(=O)Nc5ccc(OCCNCC6CCN(c7ccc(C8CCC(=O)NC8=O)cc7)CC6)cc54)CC3)cc12] | MPro |
| 223 | 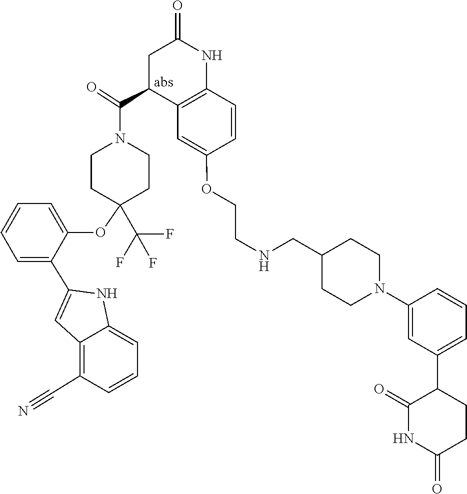 [SMILES: N#Cc1cccc2[nH]c(-c3ccccc3OC3(C(F)(F)F)CCN(C(=O)[C@H]4CC(=O)Nc5ccc(OCCNCC6CCN(c7cccc(C8CCC(=O)NC8=O)c7)CC6)cc54)CC3)cc12] | MPro |
| 224 | 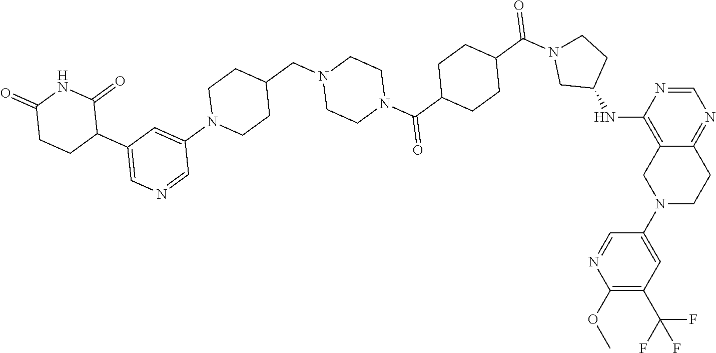 [SMILES: COc1ncc(N2CCc3ncnc(N[C@H]4CCN(C(=O)C5CCC(C(=O)N6CCN(CC7CCN(c8cncc(C9CCC(=O)NC9=O)c8)CC7)CC6)CC5)C4)c3C2)cc1C(F)(F)F] | PI3K Delta |
| 225 | 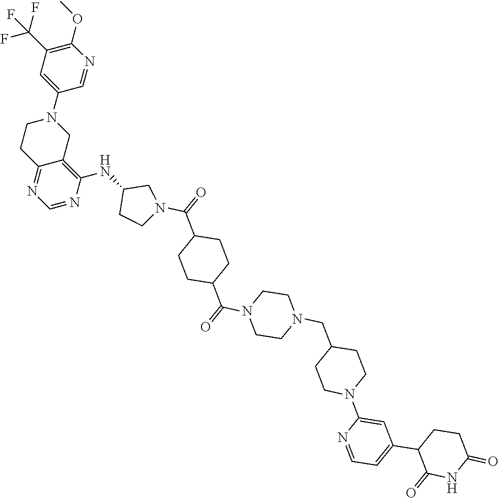 [SMILES: COc1ncc(N2CCc3ncnc(N[C@H]4CCN(C(=O)C5CCC(C(=O)N6CCN(CC7CCN(c8cc(C9CCC(=O)NC9=O)ccn8)CC7)CC6)CC5)C4)c3C2)cc1C(F)(F)F] | PI3K Delta |
| 226 | 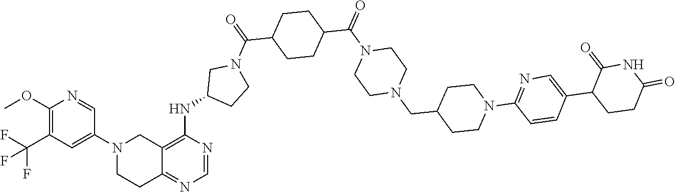 [SMILES: COc1ncc(N2CCc3ncnc(N[C@H]4CCN(C(=O)C5CCC(C(=O)N6CCN(CC7CCN(c8ccc(C9CCC(=O)NC9=O)cn8)CC7)CC6)CC5)C4)c3C2)cc1C(F)(F)F] | PI3K Delta |
| 227 | 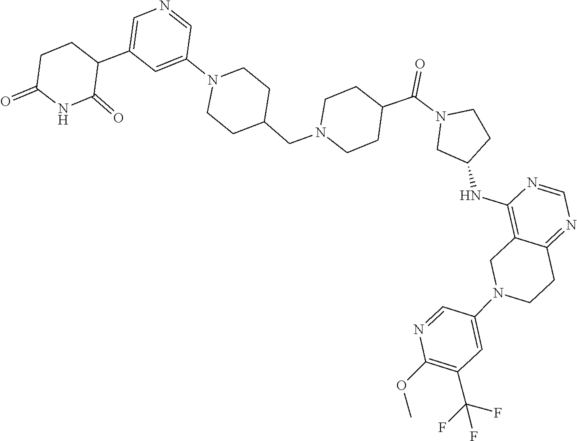 [SMILES: COc1ncc(N2CCc3ncnc(N[C@H]4CCN(C(=O)C5CCN(CC6CCN(c7cncc(C8CCC(=O)NC8=O)c7)CC6)CC5)C4)c3C2)cc1C(F)(F)F] | PI3K Delta |
| 228 | 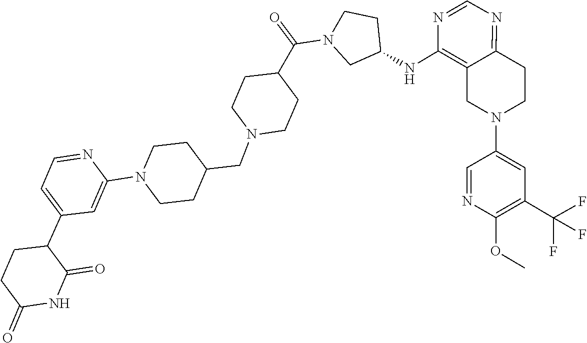 [SMILES: COc1ncc(N2CCc3ncnc(N[C@H]4CCN(C(=O)C5CCN(CC6CCN(c7cc(C8CCC(=O)NC8=O)ccn7)CC6)CC5)C4)c3C2)cc1C(F)(F)F] | PI3K Delta |
| 229 | 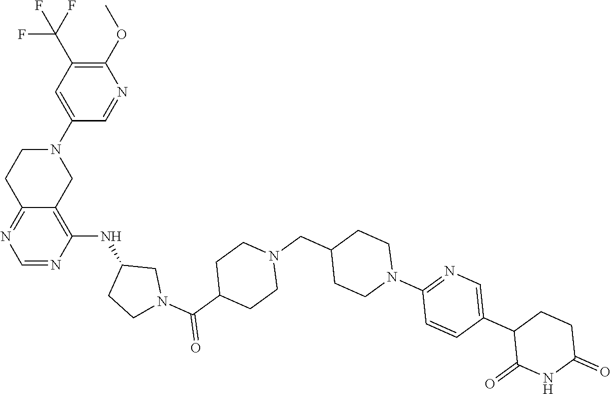 [SMILES: COc1ncc(N2CCc3ncnc(N[C@H]4CCN(C(=O)C5CCN(CC6CCN(c7ccc(C8CCC(=O)NC8=O)cn7)CC6)CC5)C4)c3C2)cc1C(F)(F)F] | PI3K Delta |
| 230 | 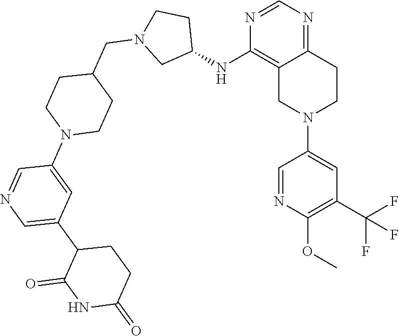 [SMILES: COc1ncc(N2CCc3ncnc(N[C@H]4CCN(CC5CCN(c6cncc(C7CCC(=O)NC7=O)c6)CC5)C4)c3C2)cc1C(F)(F)F] | PI3K Delta |
| 231 | 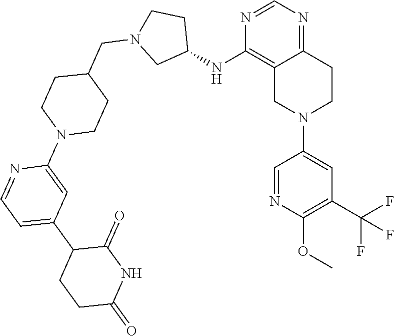 [SMILES: COc1ncc(N2CCc3ncnc(N[C@H]4CCN(CC5CCN(c6cc(C7CCC(=O)NC7=O)ccn6)CC5)C4)c3C2)cc1C(F)(F)F] | PI3K Delta |
| 232 | 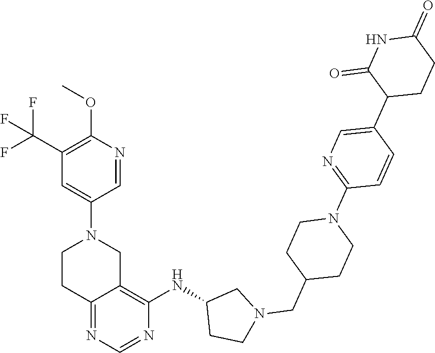 [SMILES: COc1ncc(N2CCc3ncnc(N[C@H]4CCN(CC5CCN(c6ccc(C7CCC(=O)NC7=O)cn6)CC5)C4)c3C2)cc1C(F)(F)F] | PI3K Delta |
| 233 | 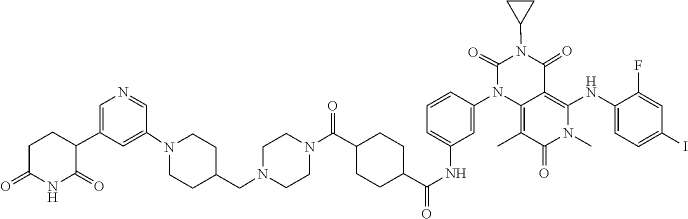 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7cncc(C8CCC(=O)NC8=O)c7)CC6)CC5)CC4)c3)c12] | MEK |
| 234 | 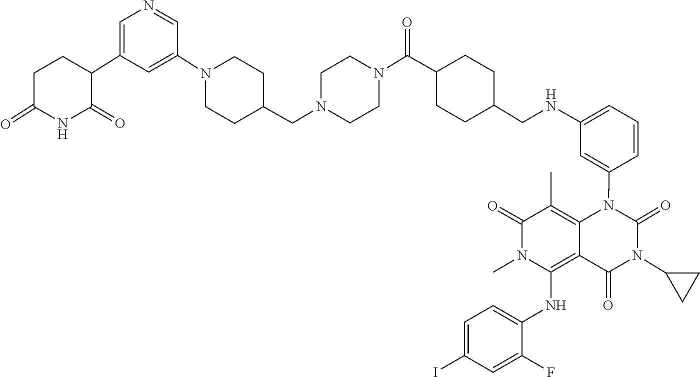 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7cncc(C8CCC(=O)NC8=O)c7)CC6)CC5)CC4)c3)c12] | MEK |
| 235 | 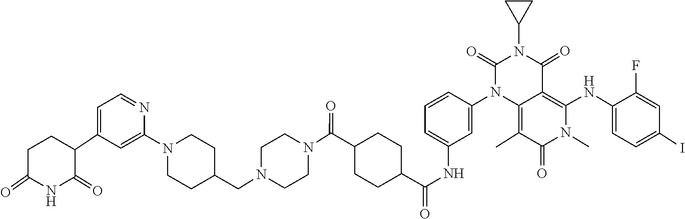 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7cc(C8CCC(=O)NC8=O)ccn7)CC6)CC5)CC4)c3)c12] | MEK |
| 236 | 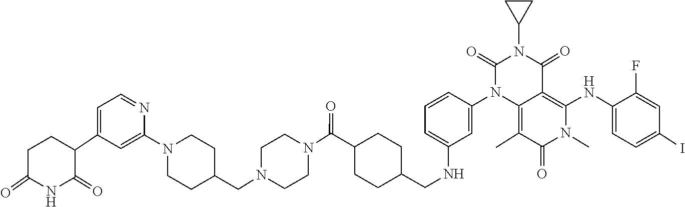 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7cc(C8CCC(=O)NC8=O)ccn7)CC6)CC5)CC4)c3)c12] | MEK |
| 237 | 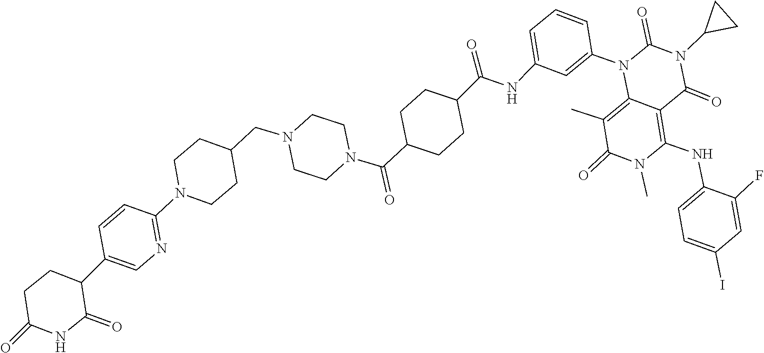 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7ccc(C8CCC(=O)NC8=O)cn7)CC6)CC5)CC4)c3)c12] | MEK |
| 238 | 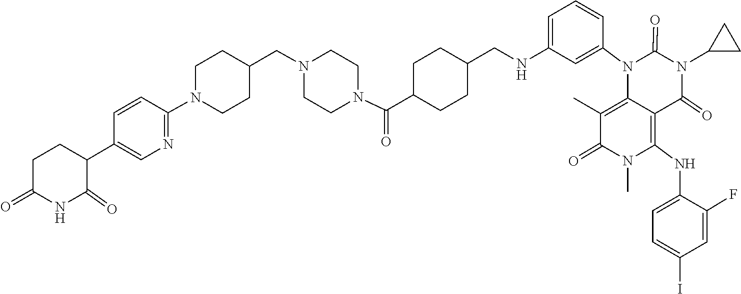 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7ccc(C8CCC(=O)NC8=O)cn7)CC6)CC5)CC4)c3)c12] | MEK |
| 239 | 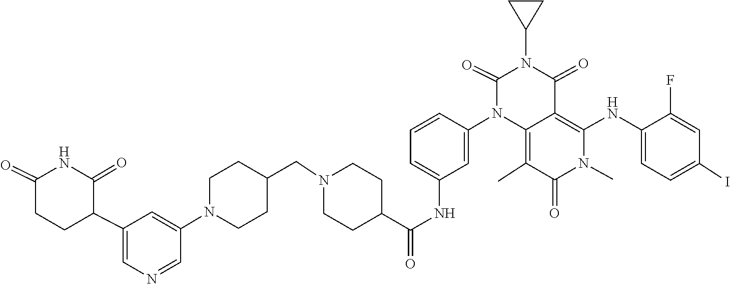 [SMILES: Cc1c(=O)n(C)c(Nc2ccc(I)cc2F)c2c(=O)n(C3CC3)c(=O)n(-c3cccc(NC(=O)C4CCN(CC5CCN(c6cncc(C7CCC(=O)NC7=O)c6)CC5)CC4)c3)c12] | MEK |
| 240 | 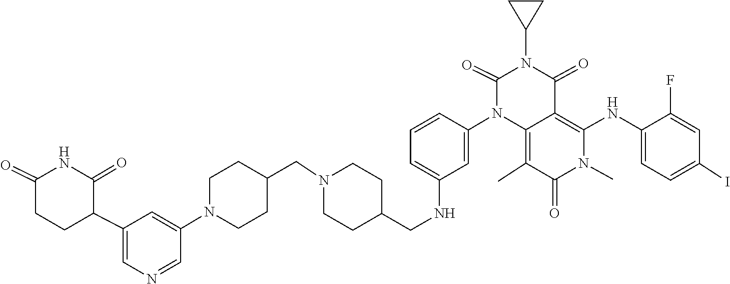 [SMILES: Cc1c(=O)n(C)c(Nc2ccc(I)cc2F)c2c(=O)n(C3CC3)c(=O)n(-c3cccc(NCC4CCN(CC5CCN(c6cncc(C7CCC(=O)NC7=O)c6)CC5)CC4)c3)c12] | MEK |
| 241 | 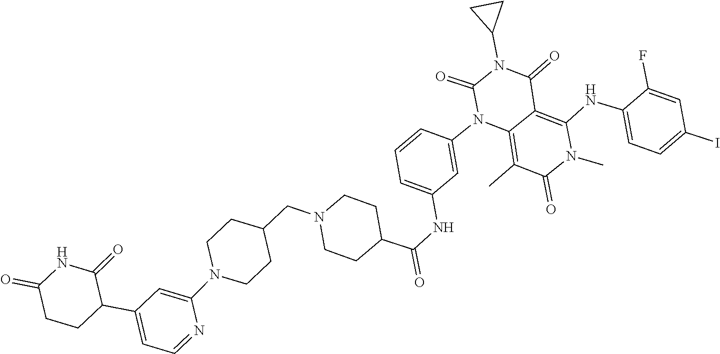 [SMILES: Cc1c(=O)n(C)c(Nc2ccc(I)cc2F)c2c(=O)n(C3CC3)c(=O)n(-c3cccc(NC(=O)C4CCN(CC5CCN(c6cc(C7CCC(=O)NC7=O)ccn6)CC5)CC4)c3)c12] | MEK |
| 242 | 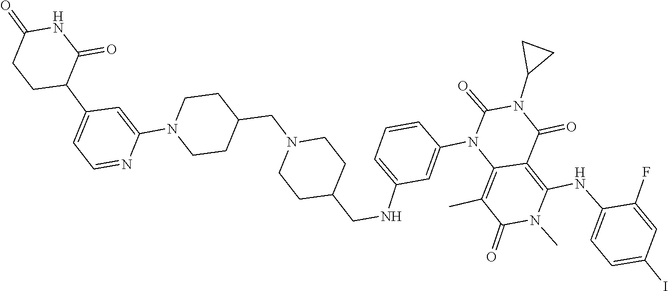 [SMILES: Cc1c(=O)n(C)c(Nc2ccc(I)cc2F)c2c(=O)n(C3CC3)c(=O)n(-c3cccc(NCC4CCN(CC5CCN(c6cc(C7CCC(=O)NC7=O)ccn6)CC5)CC4)c3)c12] | MEK |
| 243 | 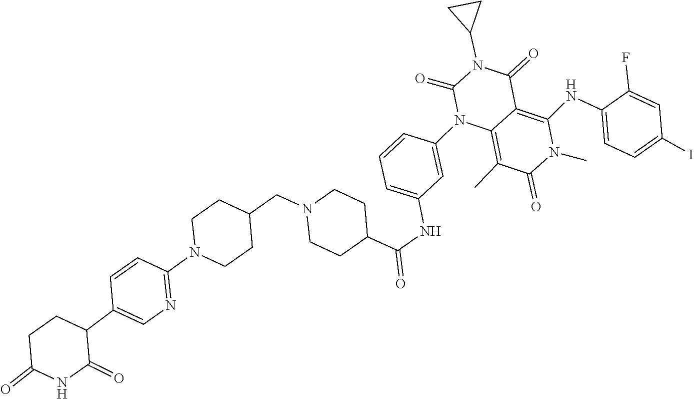 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] | MEK |
| 244 | 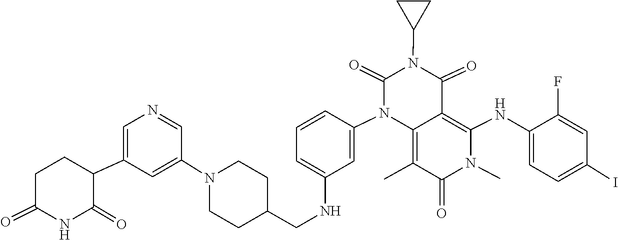 [SMILES: Cc1c(=O)n(C)c(Nc2ccc(I)cc2F)c2c(=O)n(C3CC3)c(=O)n(-c3cccc(NCC4CCN(c5cncc(C6CCC(=O)NC6=O)c5)CC4)c3)c12] | MEK |
| 245 | 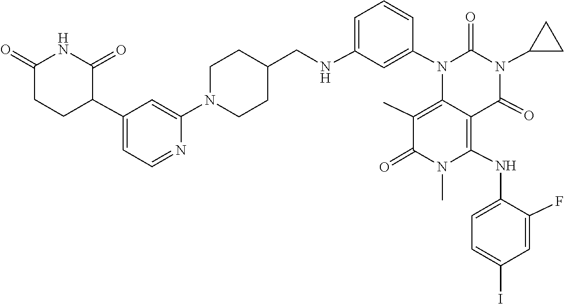 [SMILES: Cc1c(=O)n(C)c(Nc2ccc(I)cc2F)c2c(=O)n(C3CC3)c(=O)n(-c3cccc(NCC4CCN(c5cc(C6CCC(=O)NC6=O)ccn5)CC4)c3)c12] | MEK |
| 246 | 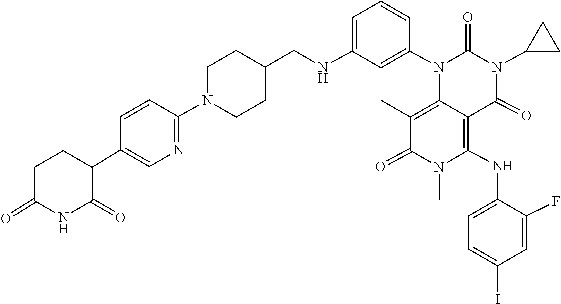 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cn5)CC4)c3)c12] | MEK |
| 247 | 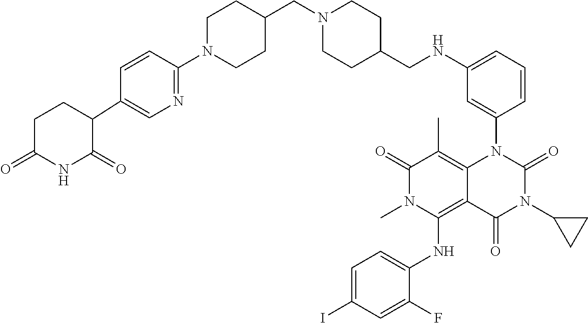 [SMILES: CCN(CCC(C)CN1CCC(CNc2cccc(-n3c(=O)n(C4CC4)c(=O)c4c(Nc5ccc(I)cc5F)n(C)c(=O)c(C)c43)c2)CC1)c1ccc(C2CCC(=O)NC2=O)cn1] | MEK |
| 248 | 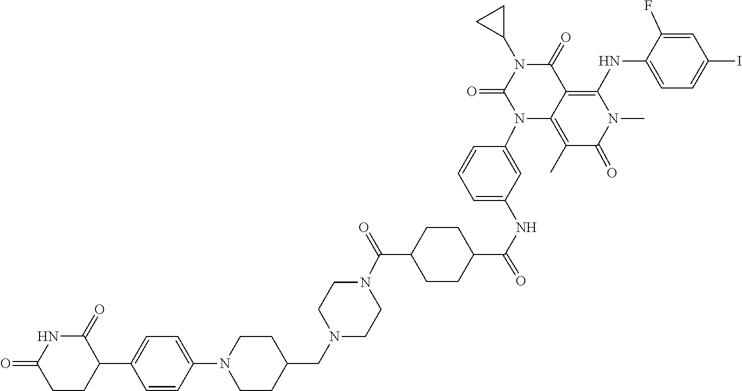 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7ccc(C8CCC(=O)NC8=O)cc7)CC6)CC5)CC4)c3)c12] | MEK |
| 249 | 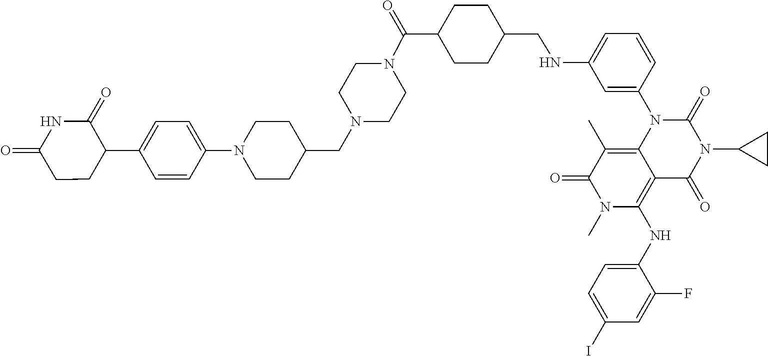 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7ccc(C8CCC(=O)NC8=O)cc7)CC6)CC5)CC4)c3)c12] | MEK |
| 250 | 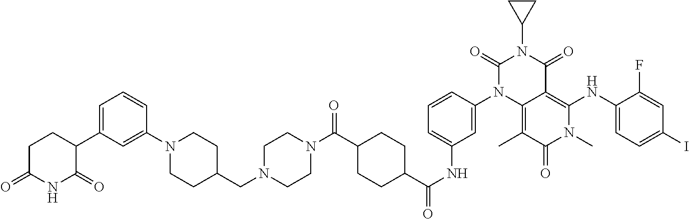 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7cccc(C8CCC(=O)NC8=O)c7)CC6)CC5)CC4)c3)c12] | MEK |
| 251 | 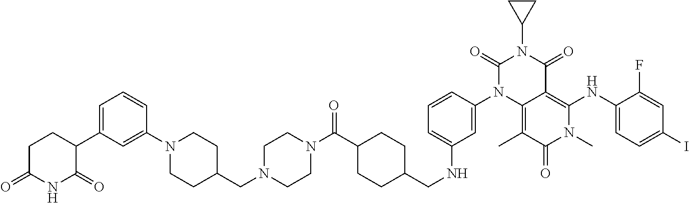 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7cccc(C8CCC(=O)NC8=O)c7)CC6)CC5)CC4)c3)c12] | MEK |
| 252 | 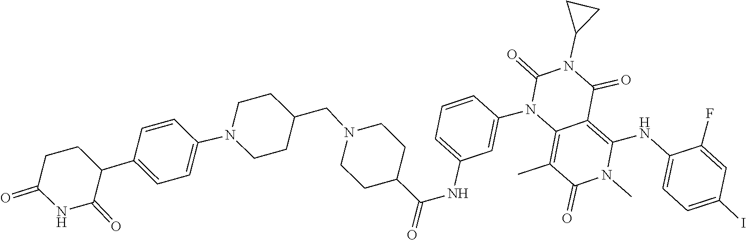 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cc6)CC5)CC4)c3)c12] | MEK |
| 253 | 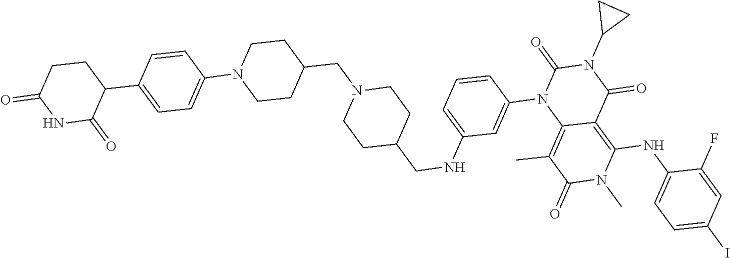 [SMILES: Cc1c(=O)n(C)c(Nc2ccc(I)cc2F)c2c(=O)n(C3CC3)c(=O)n(-c3cccc(NCC4CCN(CC5CCN(c6ccc(C7CCC(=O)NC7=O)cc6)CC5)CC4)c3)c12] | MEK |
| 254 | 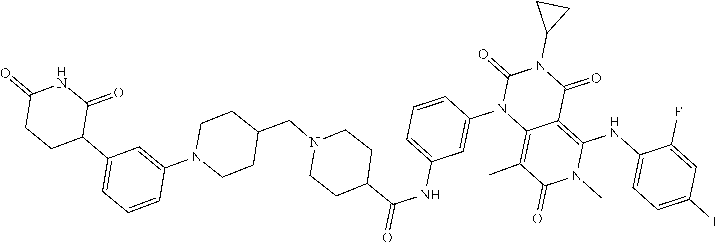 [SMILES: Cc1c(=O)n(C)c(Nc2ccc(I)cc2F)c2c(=O)n(C3CC3)c(=O)n(-c3cccc(NC(=O)C4CCN(CC5CCN(c6cccc(C7CCC(=O)NC7=O)c6)CC5)CC4)c3)c12] | MEK |
| 255 | 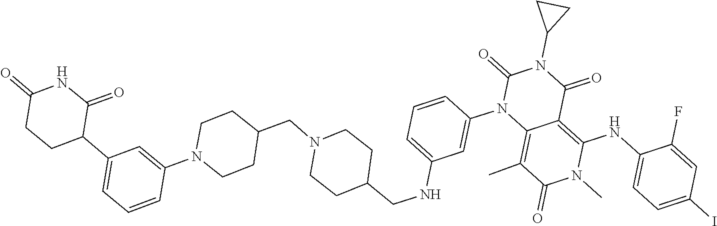 [SMILES: Cc1c(=O)n(C)c(Nc2ccc(I)cc2F)c2c(=O)n(C3CC3)c(=O)n(-c3cccc(NCC4CCN(CC5CCN(c6cccc(C7CCC(=O)NC7=O)c6)CC5)CC4)c3)c12] | MEK |
| 256 | 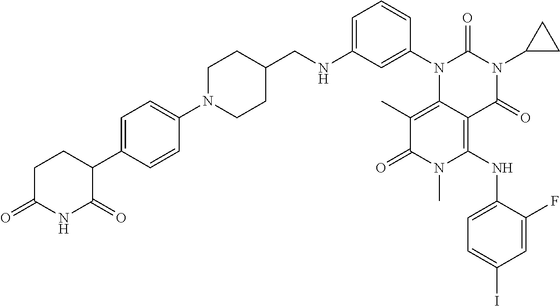 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cc5)CC4)c3)c12] | MEK |
| 257 | 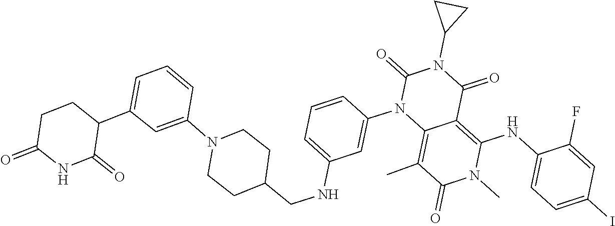 [SMILES: Cc1c(=O)n(C)c(Nc2ccc(I)cc2F)c2c(=O)n(C3CC3)c(=O)n(-c3cccc(NCC4CCN(c5cccc(C6CCC(=O)NC6=O)c5)CC4)c3)c12] | MEK |
| 258 | 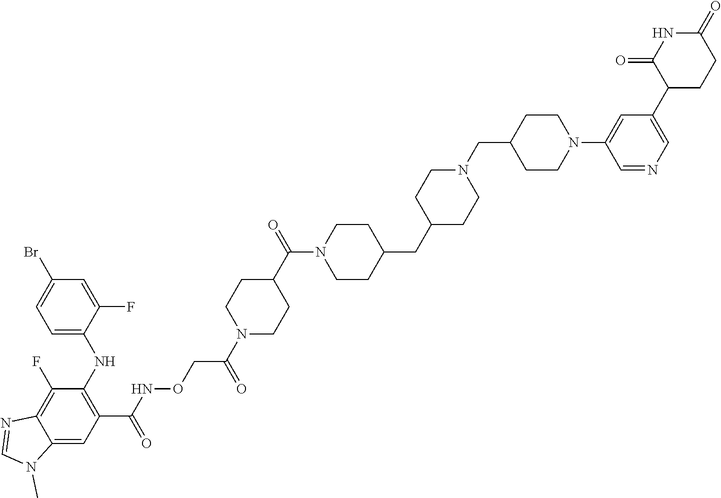 [SMILES: Cn1cnc2c(F)c(Nc3ccc(Br)cc3F)c(C(=O)NOCC(=O)N3CCC(C(=O)N4CCC(CC5CCN(CC6CCN(c7cncc(C8CCC(=O)NC8=O)c7)CC6)CC5)CC4)CC3)cc21] | MEK |
| 259 | 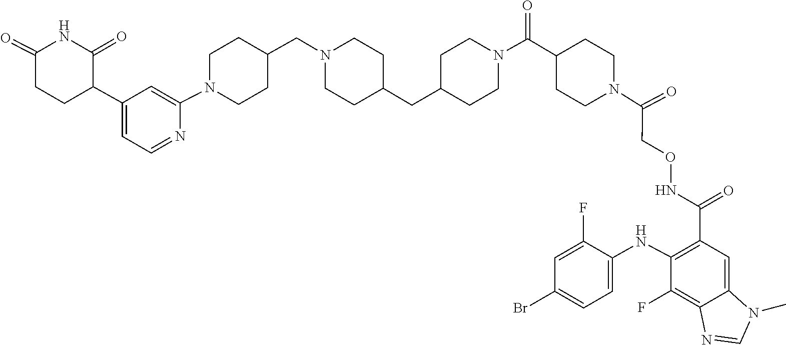 [SMILES: Cn1cnc2c(F)c(Nc3ccc(Br)cc3F)c(C(=O)NOCC(=O)N3CCC(C(=O)N4CCC(CC5CCN(CC6CCN(c7cc(C8CCC(=O)NC8=O)ccn7)CC6)CC5)CC4)CC3)cc21] | MEK |
| 260 | 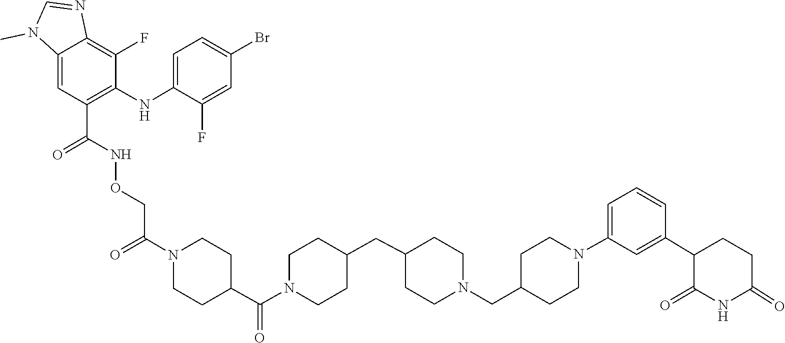 [SMILES: Cn1cnc2c(F)c(Nc3ccc(Br)cc3F)c(C(=O)NOCC(=O)N3CCC(C(=O)N4CCC(CC5CCN(CC6CCN(c7cccc(C8CCC(=O)NC8=O)c7)CC6)CC5)CC4)CC3)cc21] | MEK |
| 261 | 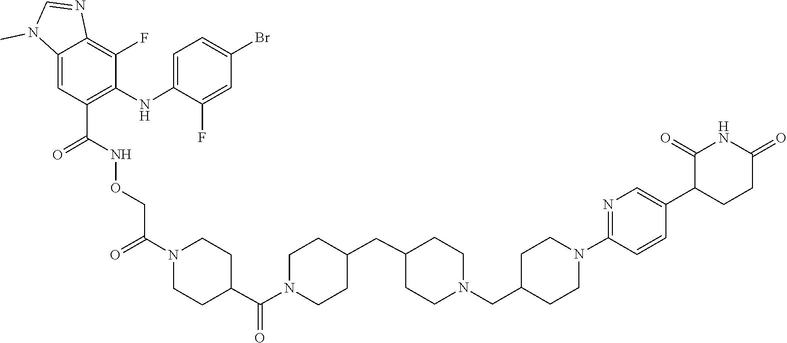 [SMILES: Cn1cnc2c(F)c(Nc3ccc(Br)cc3F)c(C(=O)NOCC(=O)N3CCC(C(=O)N4CCC(CC5CCN(CC6CCN(c7ccc(C8CCC(=O)NC8=O)cn7)CC6)CC5)CC4)CC3)cc21] | MEK |
| 262 | 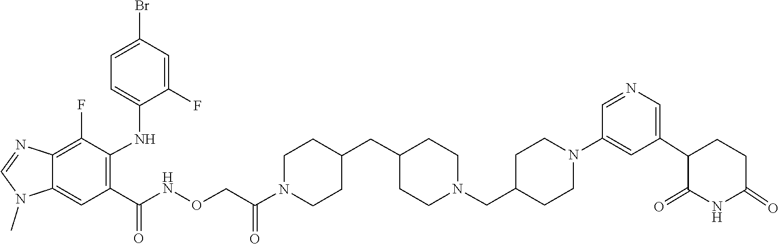 [SMILES: Cn1cnc2c(F)c(Nc3ccc(Br)cc3F)c(C(=O)NOCC(=O)N3CCC(CC4CCN(CC5CCN(c6cncc(C7CCC(=O)NC7=O)c6)CC5)CC4)CC3)cc21] | MEK |
| 263 | 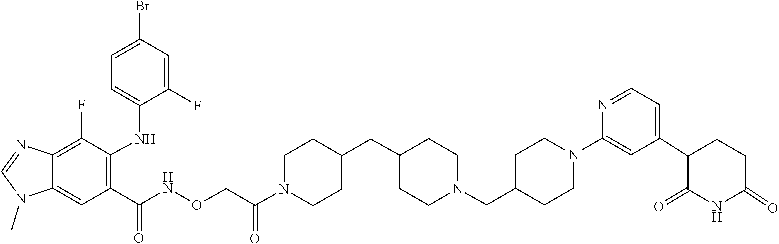 [SMILES: Cn1cnc2c(F)c(Nc3ccc(Br)cc3F)c(C(=O)NOCC(=O)N3CCC(CC4CCN(CC5CCN(c6cc(C7CCC(=O)NC7=O)ccn6)CC5)CC4)CC3)cc21] | MEK |
| 264 | 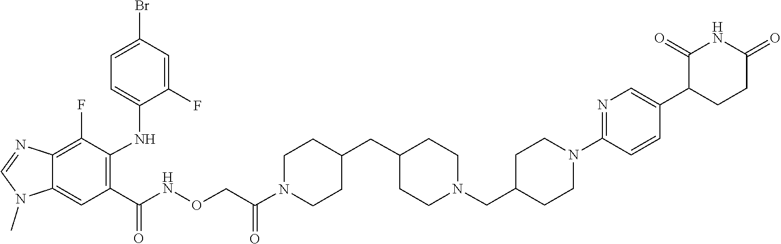 [SMILES: Cn1cnc2c(F)c(Nc3ccc(Br)cc3F)c(C(=O)NOCC(=O)N3CCC(CC4CCN(CC5CCN(c6ccc(C7CCC(=O)NC7=O)cn6)CC5)CC4)CC3)cc21] | MEK |
| 265 | 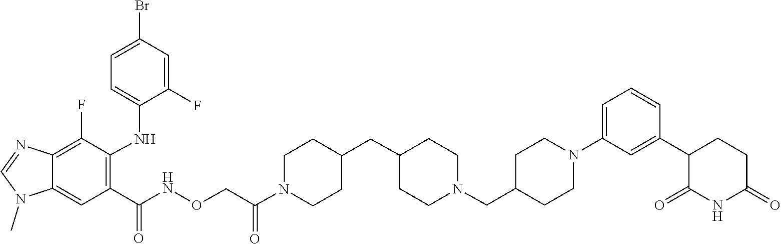 [SMILES: Cn1cnc2c(F)c(Nc3ccc(Br)cc3F)c(C(=O)NOCC(=O)N3CCC(CC4CCN(CC5CCN(c6cccc(C7CCC(=O)NC7=O)c6)CC5)CC4)CC3)cc21] | MEK |
| 266 | 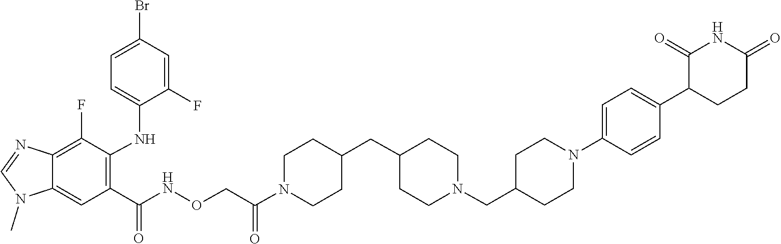 [SMILES: Cn1cnc2c(F)c(Nc3ccc(Br)cc3F)c(C(=O)NOCC(=O)N3CCC(CC4CCN(CC5CCN(c6ccc(C7CCC(=O)NC7=O)cc6)CC5)CC4)CC3)cc21] | MEK |
| 267 | 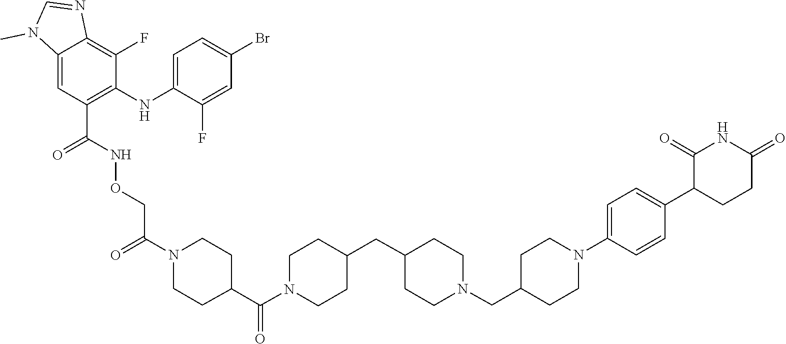 [SMILES: Cn1cnc2c(F)c(Nc3ccc(Br)cc3F)c(C(=O)NOCC(=O)N3CCC(C(=O)N4CCC(CC5CCN(CC6CCN(c7ccc(C8CCC(=O)NC8=O)cc7)CC6)CC5)CC4)CC3)cc21] | MEK |
| 268 | 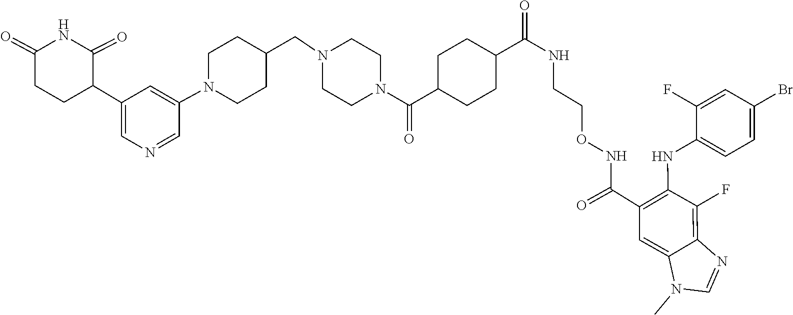 [SMILES: Cn1cnc2c(F)c(Nc3ccc(Br)cc3F)c(C(=O)NOCCNC(=O)C3CCC(C(=O)N4CCN(CC5CCN(c6cncc(C7CCC(=O)NC7=O)c6)CC5)CC4)CC3)cc21] | MEK |
| 269 | 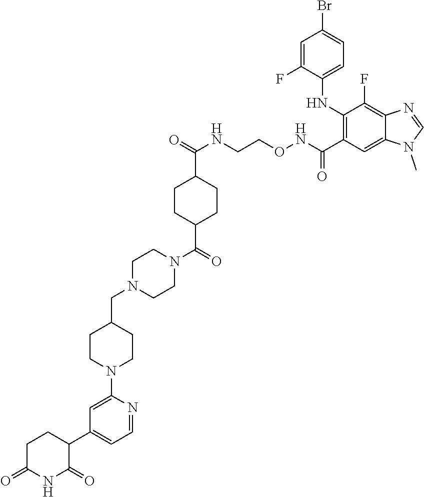 [SMILES: Cn1cnc2c(F)c(Nc3ccc(Br)cc3F)c(C(=O)NOCCNC(=O)C3CCC(C(=O)N4CCN(CC5CCN(c6cc(C7CCC(=O)NC7=O)ccn6)CC5)CC4)CC3)cc21] | MEK |
| 270 | 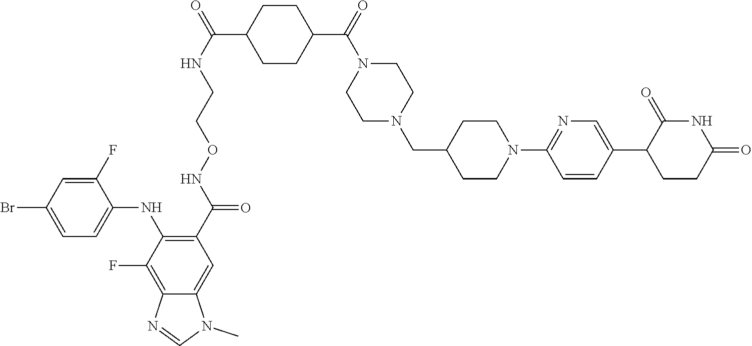 [SMILES: Cn1cnc2c(F)c(Nc3ccc(Br)cc3F)c(C(=O)NOCCNC(=O)C3CCC(C(=O)N4CCN(CC5CCN(c6ccc(C7CCC(=O)NC7=O)cn6)CC5)CC4)CC3)cc21] | MEK |
| 271 | 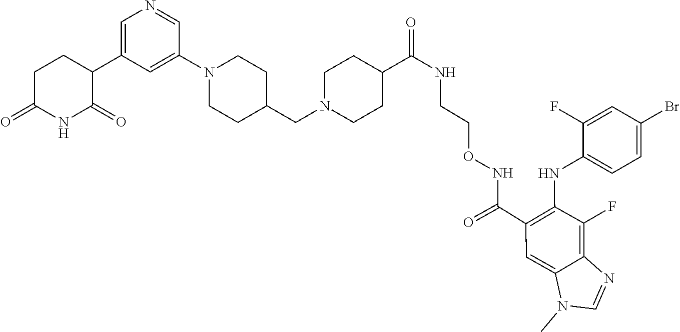 [SMILES: Cn1cnc2c(F)c(Nc3ccc(Br)cc3F)c(C(=O)NOCCNC(=O)C3CCN(CC4CCN(c5cncc(C6CCC(=O)NC6=O)c5)CC4)CC3)cc21] | MEK |
| 272 | 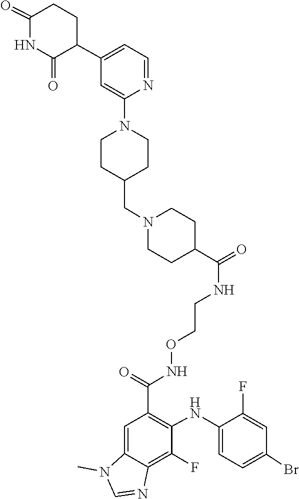 [SMILES: Cn1cnc2c(F)c(Nc3ccc(Br)cc3F)c(C(=O)NOCCNC(=O)C3CCN(CC4CCN(c5cc(C6CCC(=O)NC6=O)ccn5)CC4)CC3)cc21] | MEK |
| 273 | 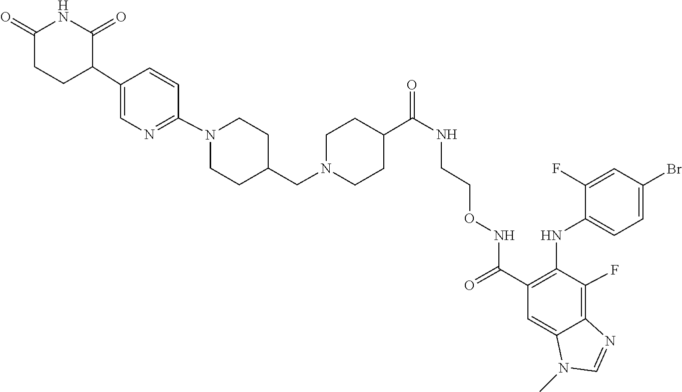 [SMILES: Cn1cnc2c(F)c(Nc3ccc(Br)cc3F)c(C(=O)NOCCNC(=O)C3CCN(CC4CCN(c5ccc(C6CCC(=O)NC6=O)cn5)CC4)CC3)cc21] | MEK |
| 274 | 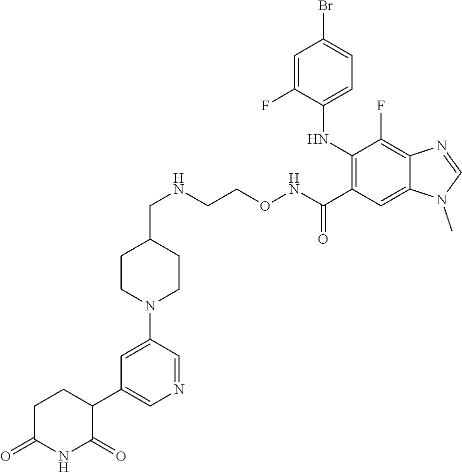 [SMILES: Cn1cnc2c(F)c(Nc3ccc(Br)cc3F)c(C(=O)NOCCNCC3CCN(c4cncc(C5CCC(=O)NC5=O)c4)CC3)cc21] | MEK |
| 275 | 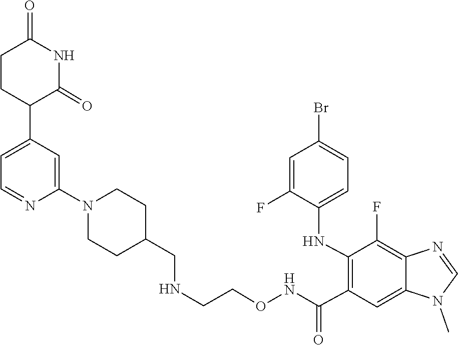 [SMILES: Cn1cnc2c(F)c(Nc3ccc(Br)cc3F)c(C(=O)NOCCNCC3CCN(c4cc(C5CCC(=O)NC5=O)ccn4)CC3)cc21] | MEK |
| 276 | 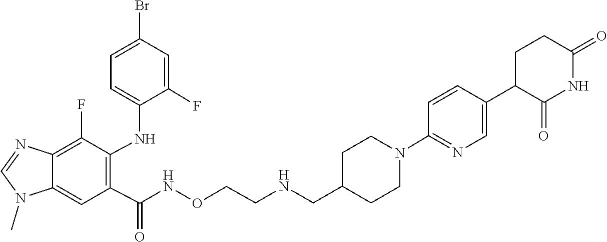 [SMILES: Cn1cnc2c(F)c(Nc3ccc(Br)cc3F)c(C(=O)NOCCNCC3CCN(c4ccc(C5CCC(=O)NC5=O)cn4)CC3)cc21] | MEK |
| 277 | 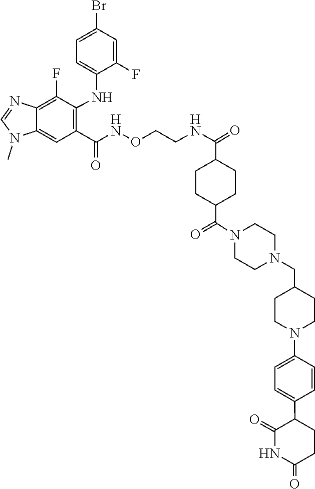 [SMILES: Cn1cnc2c(F)c(Nc3ccc(Br)cc3F)c(C(=O)NOCCNC(=O)C3CCC(C(=O)N4CCN(CC5CCN(c6ccc(C7CCC(=O)NC7=O)cc6)CC5)CC4)CC3)cc21] | MEK |
| 278 | 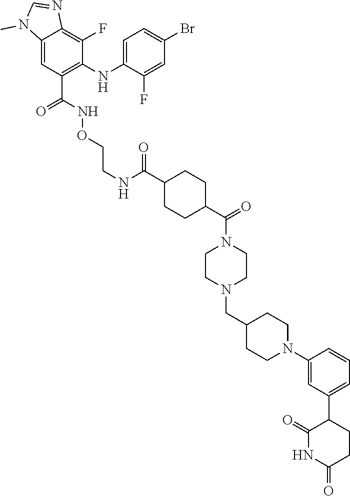 [SMILES: Cn1cnc2c(F)c(Nc3ccc(Br)cc3F)c(C(=O)NOCCNC(=O)C3CCC(C(=O)N4CCN(CC5CCN(c6cccc(C7CCC(=O)NC7=O)c6)CC5)CC4)CC3)cc21] | MEK |
| 279 | 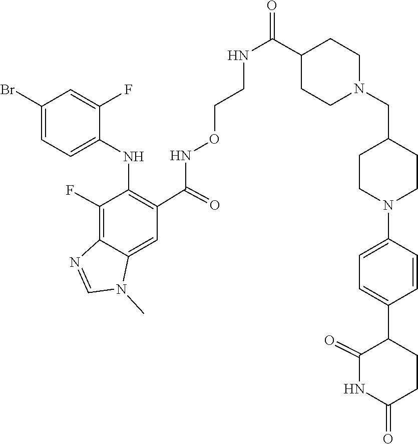 [SMILES: Cn1cnc2c(F)c(Nc3ccc(Br)cc3F)c(C(=O)NOCCNC(=O)C3CCN(CC4CCN(c5ccc(C6CCC(=O)NC6=O)cc5)CC4)CC3)cc21] | MEK |
| 280 | 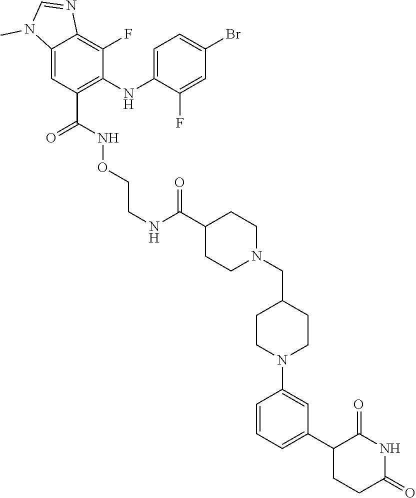 [SMILES: Cn1cnc2c(F)c(Nc3ccc(Br)cc3F)c(C(=O)NOCCNC(=O)C3CCN(CC4CCN(c5cccc(C6CCC(=O)NC6=O)c5)CC4)CC3)cc21] | MEK |
| 281 | 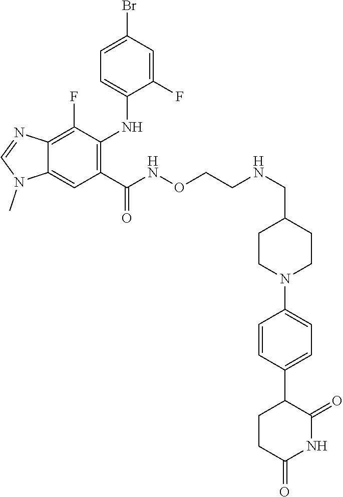 [SMILES: Cn1cnc2c(F)c(Nc3ccc(Br)cc3F)c(C(=O)NOCCNCC3CCN(c4ccc(C5CCC(=O)NC5=O)cc4)CC3)cc21] | MEK |
| 282 | 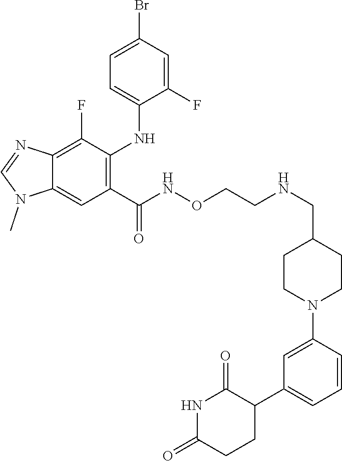 [SMILES: Cn1cnc2c(F)c(Nc3ccc(Br)cc3F)c(C(=O)NOCCNCC3CCN(c4cccc(C5CCC(=O)NC5=O)c4)CC3)cc21] | MEK |
| 283 | 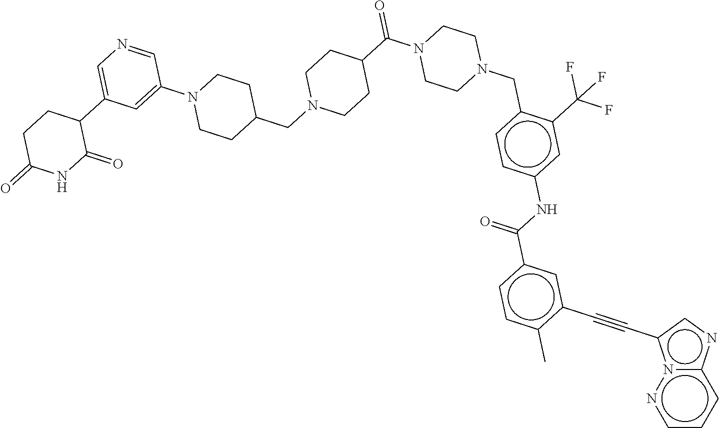 | BCR- ABL |
| 284 | 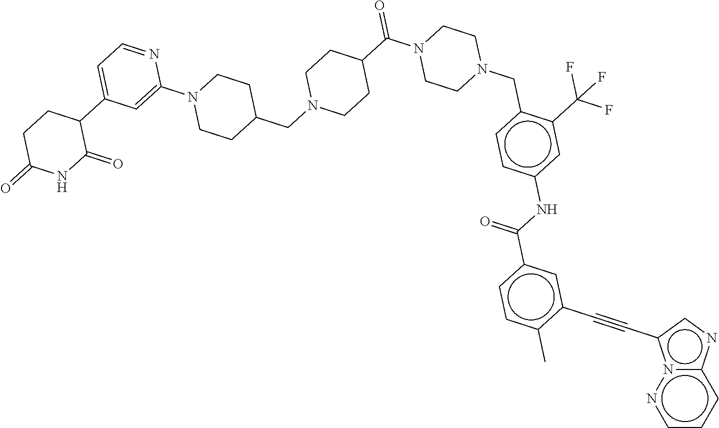 | BCR- ABL |
| 285 | 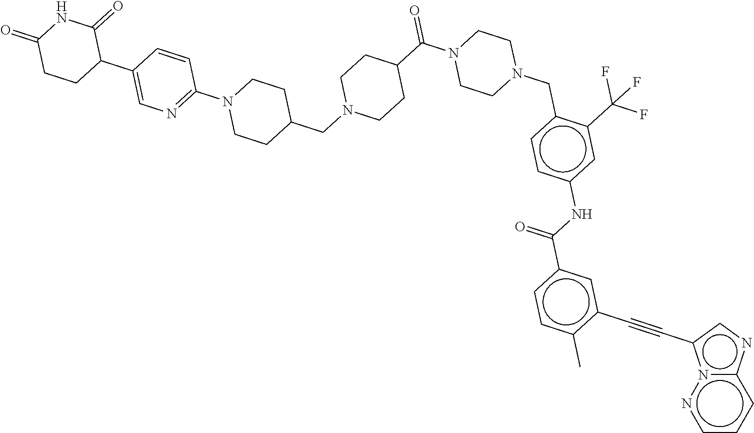 | BCR- ABL |
| 286 | 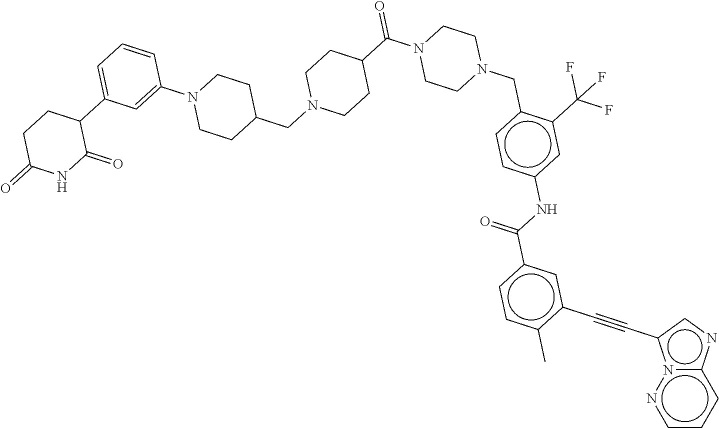 | BCR- ABL |
| 287 | 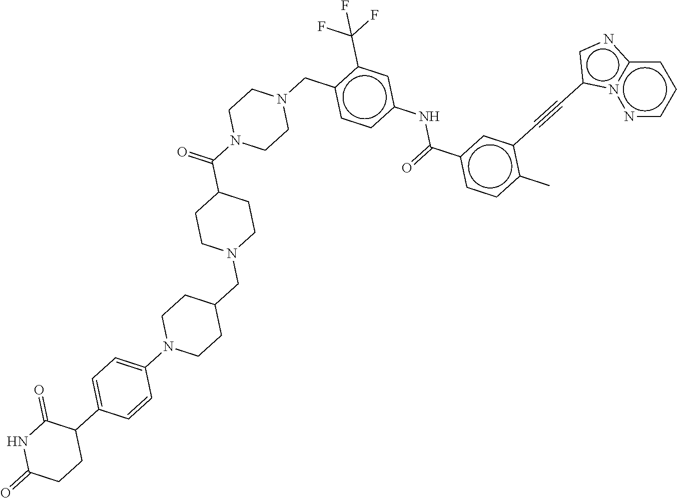 | BCR- ABL |
| 288 | 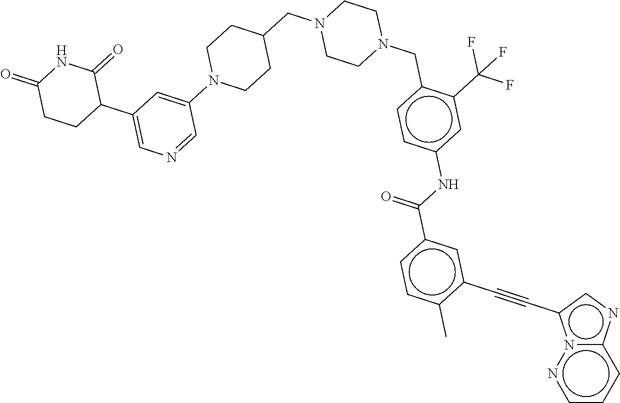 | BCR- ABL |
| 289 | 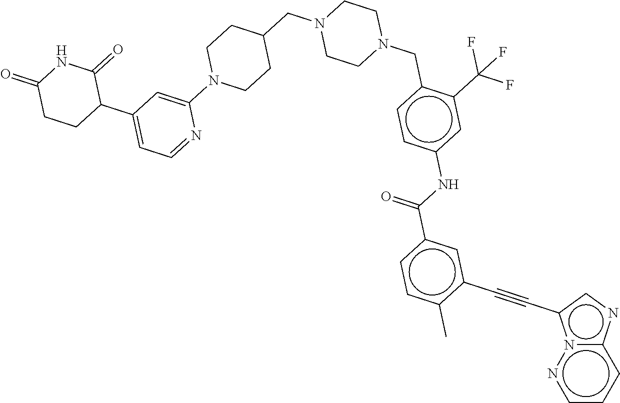 | BCR- ABL |
| 290 | 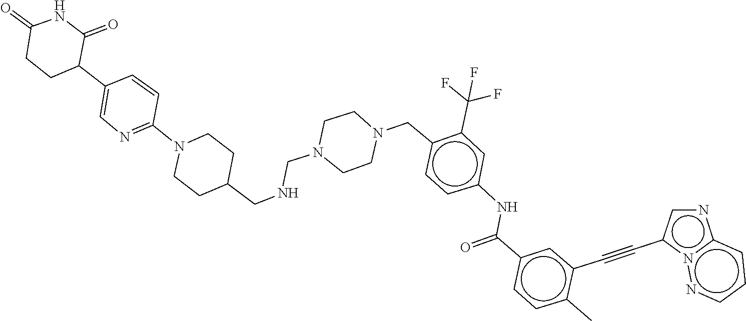 | BCR- ABL |
| 291 | 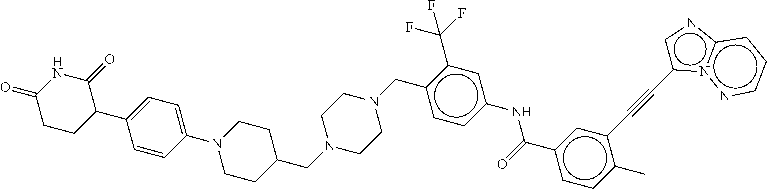 | BCR- ABL |
| 292 | 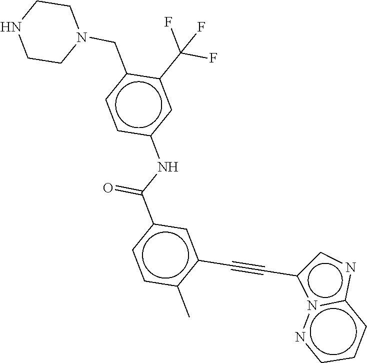 | BCR- ABL |
| 293 | 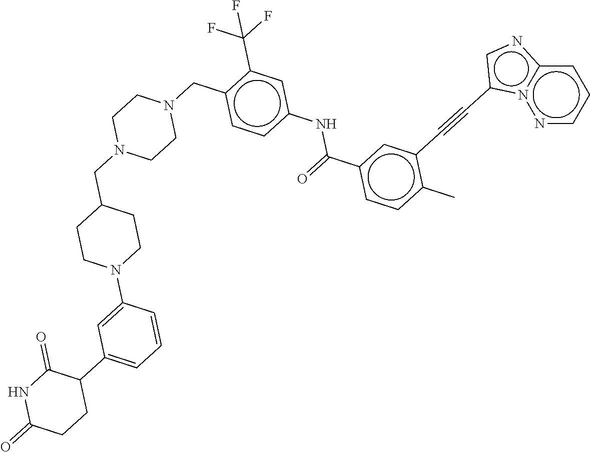 | BCR- ABL |
| 294 | 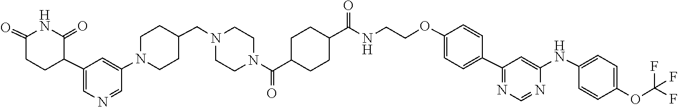 [SMILES: O=C1CCC(c2cncc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] | BCR- ABL |
| 295 | 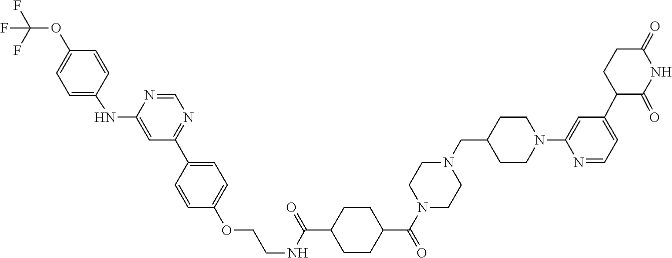 [SMILES: O=C1CCC(c2ccnc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] | BCR- ABL |
| 296 | 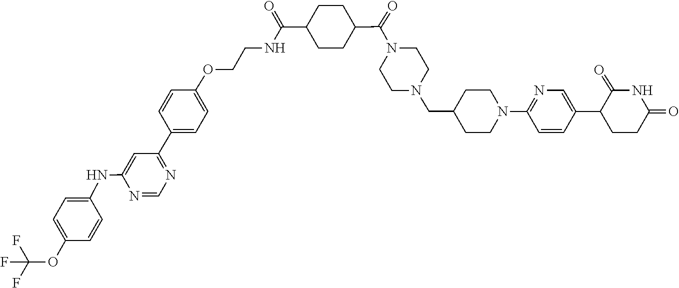 [SMILES: O=C1CCC(c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)nc2)C(=O)N1] | BCR- ABL |
| 297 | 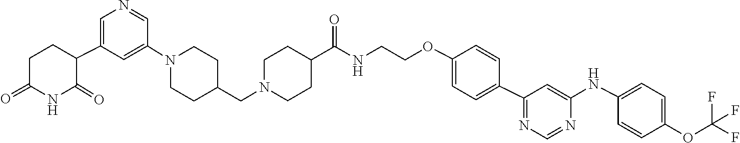 [SMILES: O=C1CCC(c2cncc(N3CCC(CN4CCC(C(=O)NCCOc5ccc(-c6cc(Nc7ccc(OC(F)(F)F)cc7)ncn6)cc5)CC4)CC3)c2)C(=O)N1] | BCR- ABL |
| 298 | 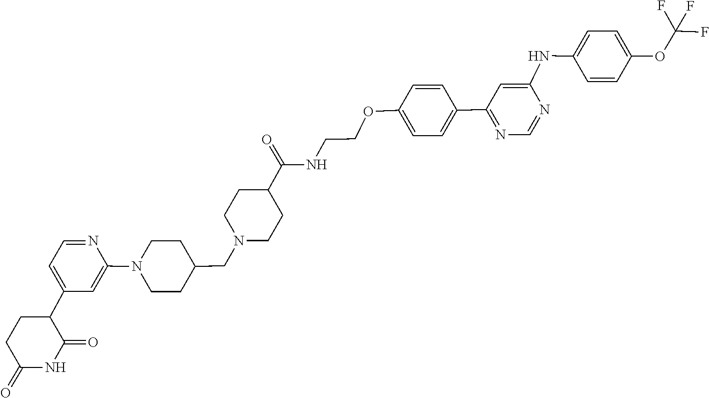 [SMILES: O=C1CCC(c2ccnc(N3CCC(CN4CCC(C(=O)NCCOc5ccc(-c6cc(Nc7ccc(OC(F)(F)F)cc7)ncn6)cc5)CC4)CC3)c2)C(=O)N1] | BCR- ABL |
| 299 | 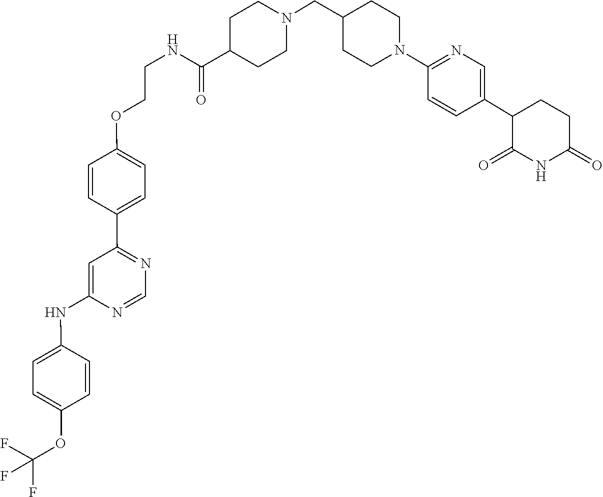 [SMILES: O=C1CCC(c2ccc(N3CCC(CN4CCC(C(=O)NCCOc5ccc(-c6cc(Nc7ccc(OC(F)(F)F)cc7)ncn6)cc5)CC4)CC3)nc2)C(=O)N1] | BCR- ABL |
| 300 | 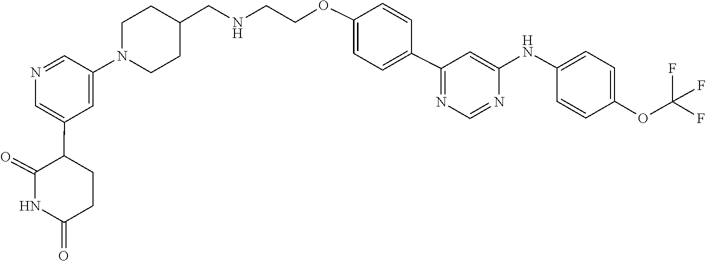 [SMILES: O=C1CCC(c2cncc(N3CCC(CNCCOc4ccc(-c5cc(Nc6ccc(OC(F)(F)F)cc6)ncn5)cc4)CC3)c2)C(=O)N1] | BCR- ABL |
| 301 | 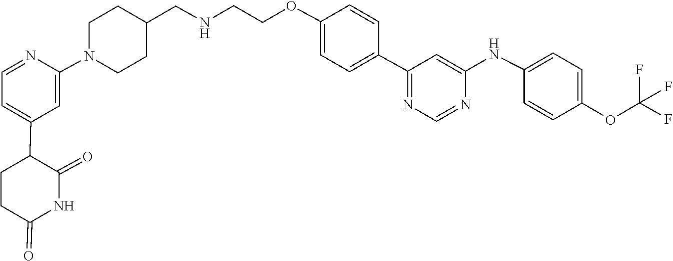 [SMILES: O=C1CCC(c2ccnc(N3CCC(CNCCOc4ccc(-c5cc(Nc6ccc(OC(F)(F)F)cc6)ncn5)cc4)CC3)c2)C(=O)N1] | BCR- ABL |
| 302 | 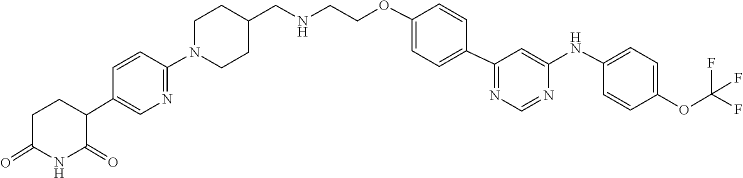 [SMILES: O=C1CCC(c2ccc(N3CCC(CNCCOc4ccc(-c5cc(Nc6ccc(OC(F)(F)F)cc6)ncn5)cc4)CC3)nc2)C(=O)N1] | BCR- ABL |
| 303 | 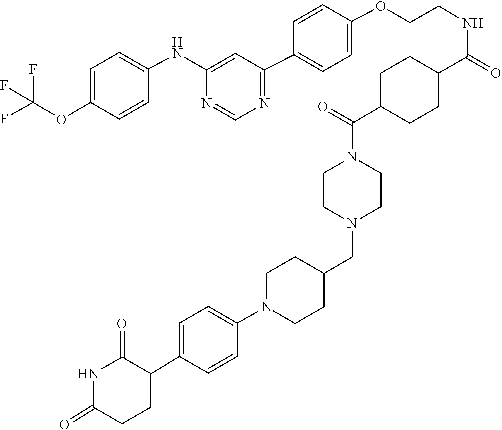 [SMILES: O=C1CCC(c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)cc2)C(=O)N1] | BCR- ABL |
| 304 | 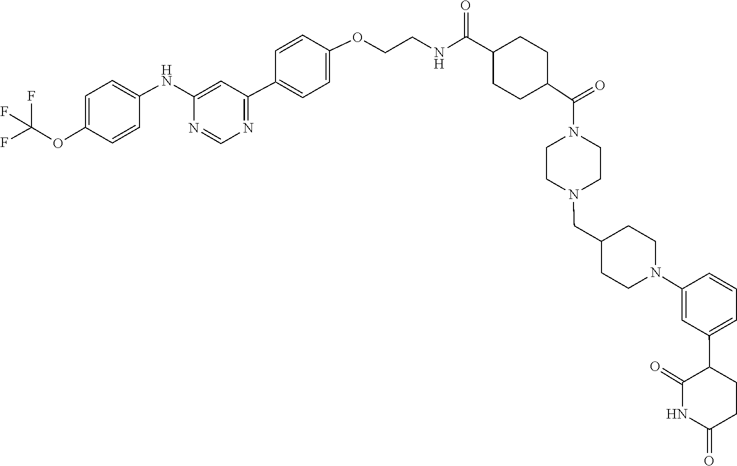 [SMILES: O=C1CCC(c2cccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] | BCR- ABL |
| 305 | 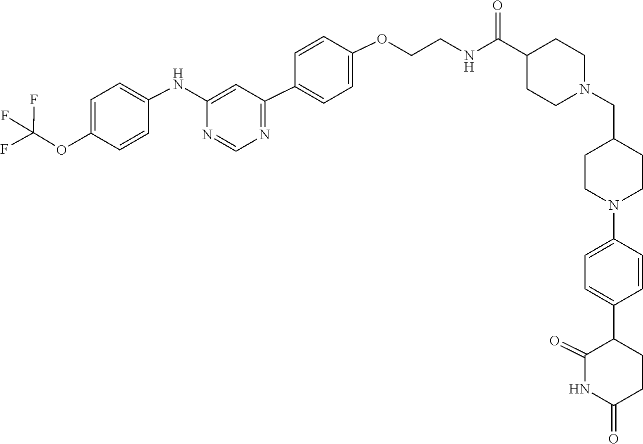 [SMILES: O=C1CCC(c2ccc(N3CCC(CN4CCC(C(=O)NCCOc5ccc(-c6cc(Nc7ccc(OC(F)(F)F)cc7)ncn6)cc5)CC4)CC3)cc2)C(=O)N1] | BCR- ABL |
| 306 | 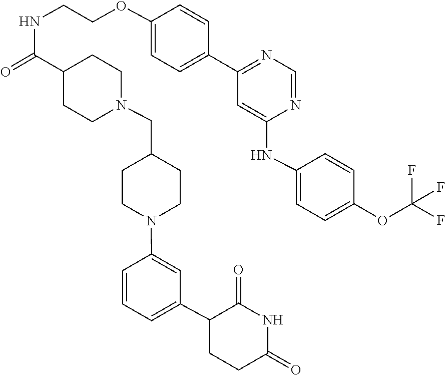 [SMILES: O=C1CCC(c2cccc(N3CCC(CN4CCC(C(=O)NCCOc5ccc(-c6cc(Nc7ccc(OC(F)(F)F)cc7)ncn6)cc5)CC4)CC3)c2)C(=O)N1] | BCR- ABL |
| 307 | 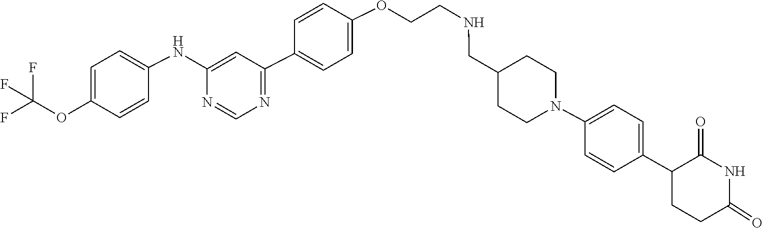 [SMILES: O=C1CCC(c2ccc(N3CCC(CNCCOc4ccc(-c5cc(Nc6ccc(OC(F)(F)F)cc6)ncn5)cc4)CC3)cc2)C(=O)N1] | BCR- ABL |
| 308 | 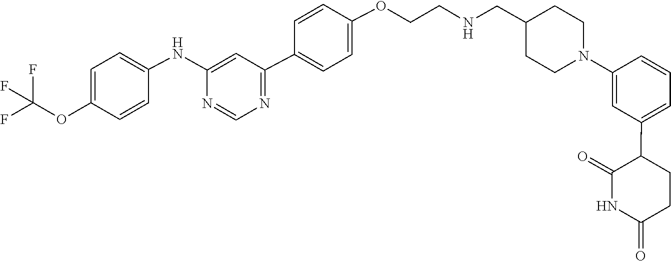 [SMILES: O=C1CCC(c2cccc(N3CCC(CNCCOc4ccc(-c5cc(Nc6ccc(OC(F)(F)F)cc6)ncn5)cc4)CC3)c2)C(=O)N1] | BCR- ABL |
| 309 | 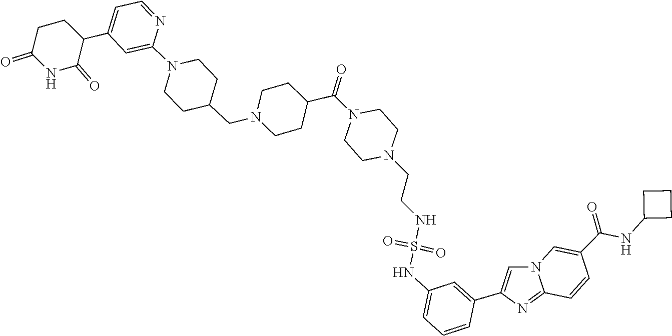 [SMILES: O=C1CCC(c2ccnc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)c2)C(=O)N1] | MLL |
| 310 | 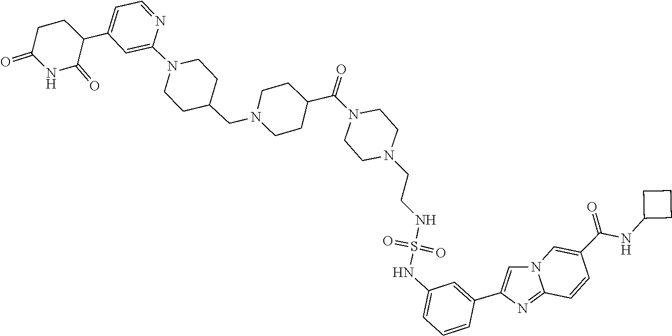 [SMILES: O=C1CCC(c2ccnc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)c2)C(=O)N1] | MLL |
| 311 | 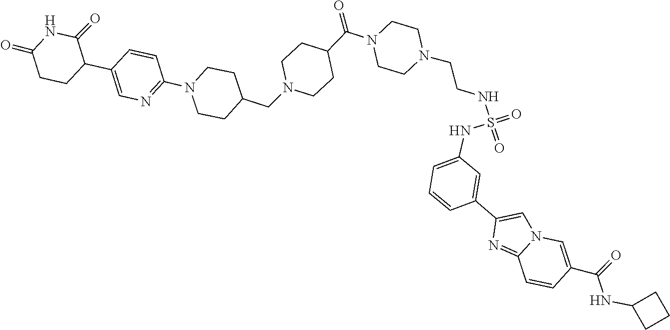 [SMILES: O=C1CCC(c2ccc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)nc2)C(=O)N1] | MLL |
| 312 | 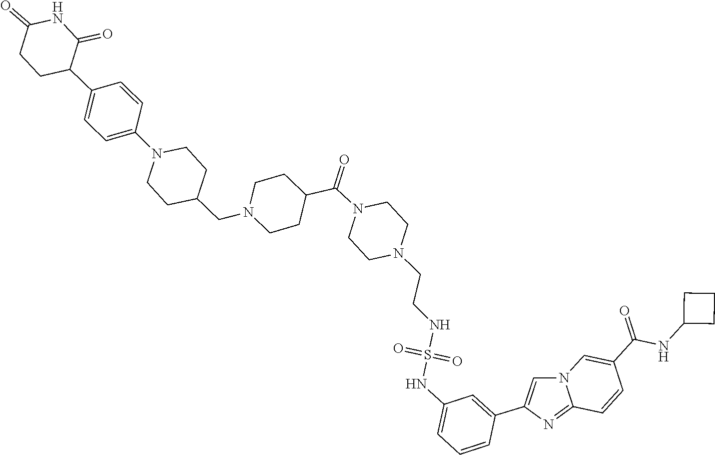 [SMILES: Cc1ccc(-c2cn3cc(C(=O)NC4CCC4)ccc3n2)cc1NS(=O)(=O)NCCN1CCN(C(=O)C2CCN(CC3CCN(c4ccc(C5CCC(=O)NC5=O)cc4)CC3)CC2)CC1] | MLL |
| 313 | 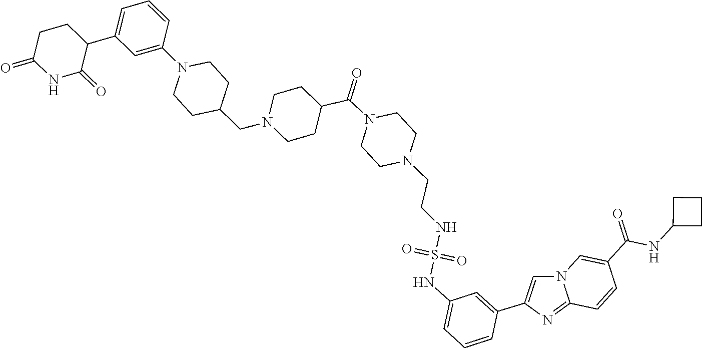 [SMILES: Cc1ccc(-c2cn3cc(C(=O)NC4CCC4)ccc3n2)cc1NS(=O)(=O)NCCN1CCN(C(=O)C2CCN(CC3CCN(c4cccc(C5CCC(=O)NC5=O)c4)CC3)CC2)CC1] | MLL |
| 314 | 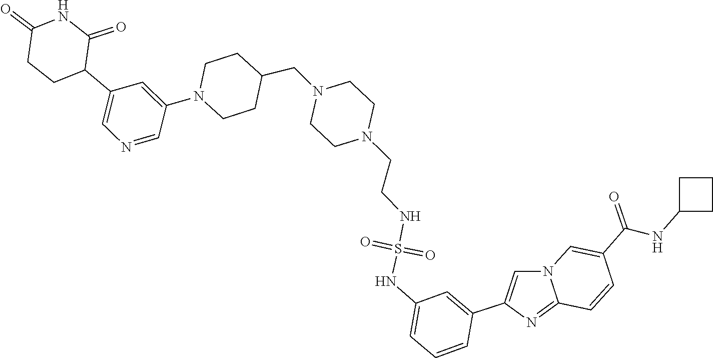 [SMILES: O=C1CCC(c2cncc(N3CCC(CN4CCN(CCNS(=O)(=O)Nc5cccc(-c6cn7cc(C(=O)NC8CCC8)ccc7n6)c5)CC4)CC3)c2)C(=O)N1] | MLL |
| 315 | 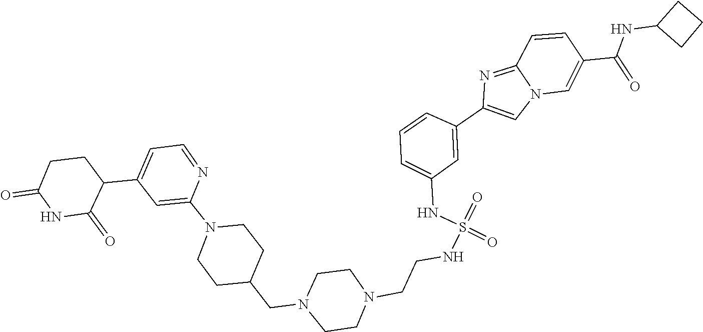 [SMILES: O=C1CCC(c2ccnc(N3CCC(CN4CCN(CCNS(=O)(=O)Nc5cccc(-c6cn7cc(C(=O)NC8CCC8)ccc7n6)c5)CC4)CC3)c2)C(=O)N1] | MLL |
| 316 | 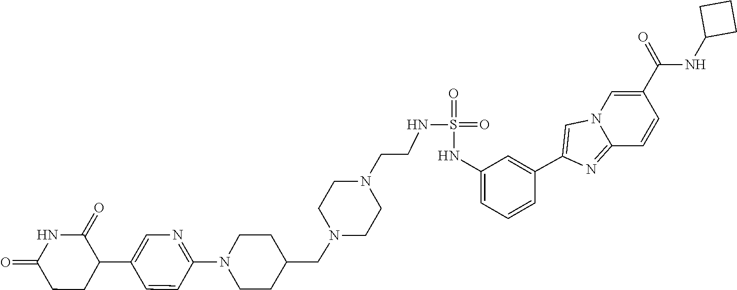 [SMILES: O=C1CCC(c2ccc(N3CCC(CN4CCN(CCNS(=O)(=O)Nc5cccc(-c6cn7cc(C(=O)NC8CCC8)ccc7n6)c5)CC4)CC3)nc2)C(=O)N1] | MLL |
| 317 | 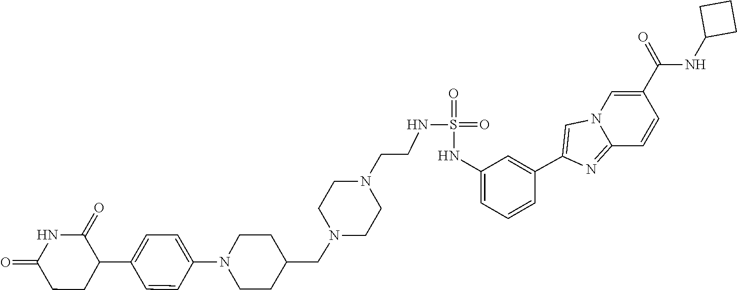 [SMILES: O=C1CCC(c2ccc(N3CCC(CN4CCN(CCNS(=O)(=O)Nc5cccc(-c6cn7cc(C(=O)NC8CCC8)ccc7n6)c5)CC4)CC3)cc2)C(=O)N1] | MLL |
| 318 | 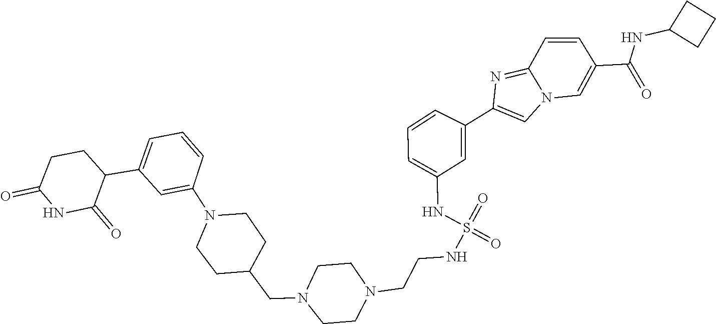 [SMILES: O=C1CCC(c2cccc(N3CCC(CN4CCN(CCNS(=O)(=O)Nc5cccc(-c6cn7cc(C(=O)NC8CCC8)ccc7n6)c5)CC4)CC3)c2)C(=O)N1] | MLL |
| 319 | 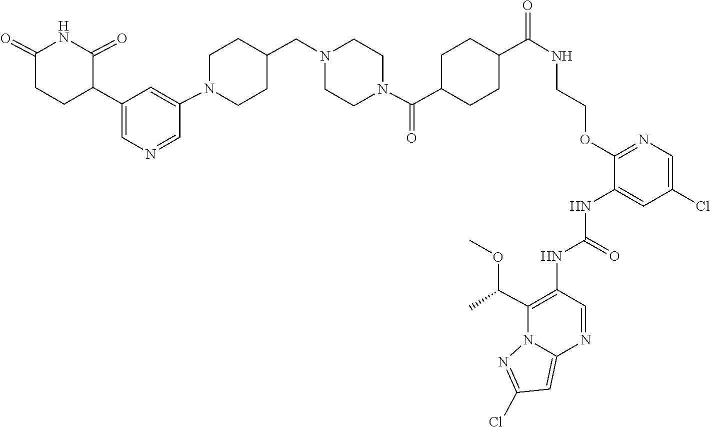 [SMILES: CO[C@@H](C)c1c(NC(=O)Nc2cc(Cl)cnc2OCCNC(=O)C2CCC(C(=O)N3CCN(CC4CCN(c5cncc(C6CCC(=O)NC6=O)c5)CC4)CC3)CC2)cnc2cc(Cl)nn12] | MALT1 |
| 320 | 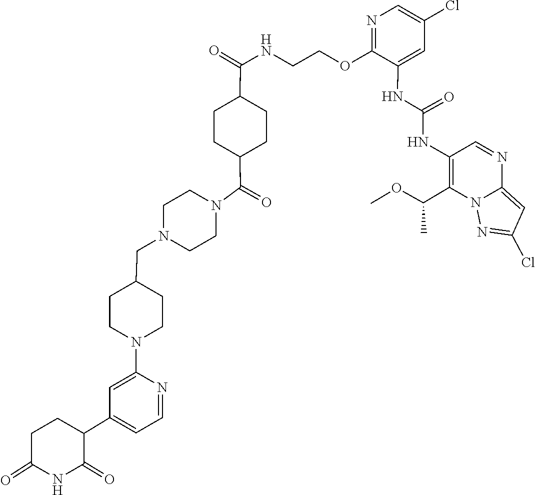 [SMILES: CO[C@@H](C)c1c(NC(=O)Nc2cc(Cl)cnc2OCCNC(=O)C2CCC(C(=O)N3CCN(CC4CCN(c5cc(C6CCC(=O)NC6=O)ccn5)CC4)CC3)CC2)cnc2cc(Cl)nn12] | MALT1 |
| 321 | 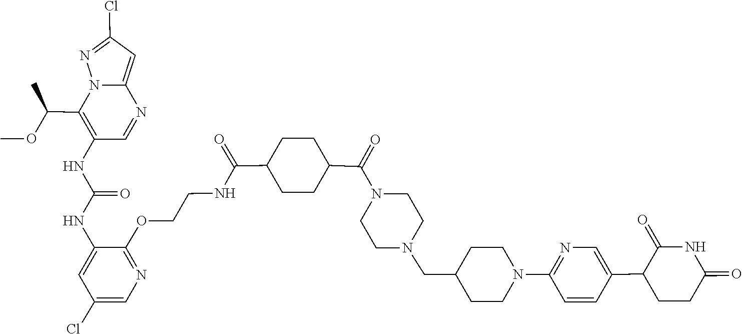 [SMILES: CO[C@@H](C)c1c(NC(=O)Nc2cc(Cl)cnc2OCCNC(=O)C2CCC(C(=O)N3CCN(CC4CCN(c5ccc(C6CCC(=O)NC6=O)cn5)CC4)CC3)CC2)cnc2cc(Cl)nn12] | MALT1 |
| 322 | 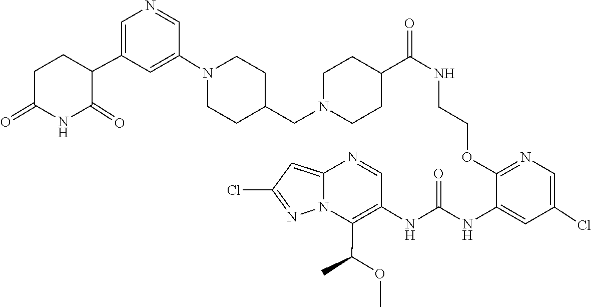 [SMILES: CO[C@@H](C)c1c(NC(=O)Nc2cc(Cl)cnc2OCCNC(=O)C2CCN(CC3CCN(c4cncc(C5CCC(=O)NC5=O)c4)CC3)CC2)cnc2cc(Cl)nn12] | MALT1 |
| 323 | 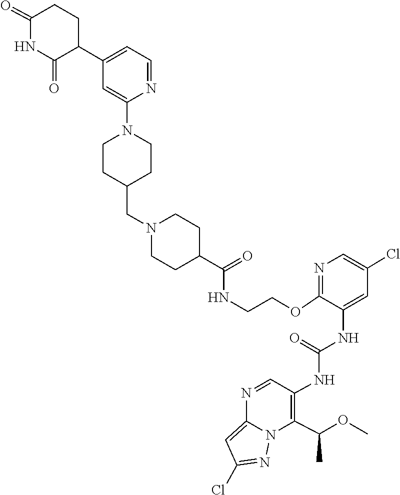 [SMILES: CO[C@@H](C)c1c(NC(=O)Nc2cc(Cl)cnc2OCCNC(=O)C2CCN(CC3CCN(c4cc(C5CCC(=O)NC5=O)ccn4)CC3)CC2)cnc2cc(Cl)nn12] | MALT1 |
| 324 | 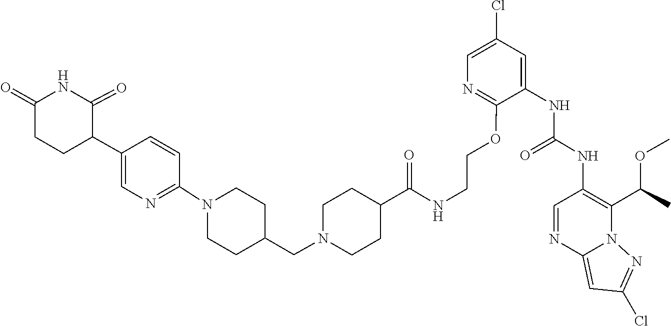 [SMILES: CO[C@@H](C)c1c(NC(=O)Nc2cc(Cl)cnc2OCCNC(=O)C2CCN(CC3CCN(c4ccc(C5CCC(=O)NC5=O)cn4)CC3)CC2)cnc2cc(Cl)nn12] | MALT1 |
| 325 | 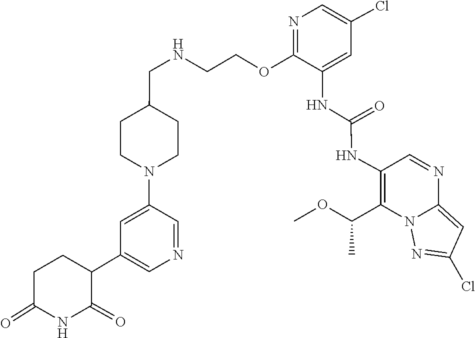 [SMILES: CO[C@@H](C)c1c(NC(=O)Nc2cc(Cl)cnc2OCCNCC2CCN(c3cncc(C4CCC(=O)NC4=O)c3)CC2)cnc2cc(Cl)nn12] | MALT1 |
| 326 | 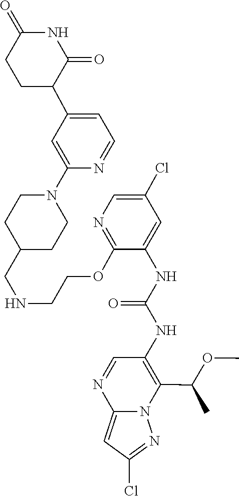 [SMILES: CO[C@@H](C)c1c(NC(=O)Nc2cc(Cl)cnc2OCCNCC2CCN(c3cc(C4CCC(=O)NC4=O)ccn3)CC2)cnc2cc(Cl)nn12] | MALT1 |
| 327 | 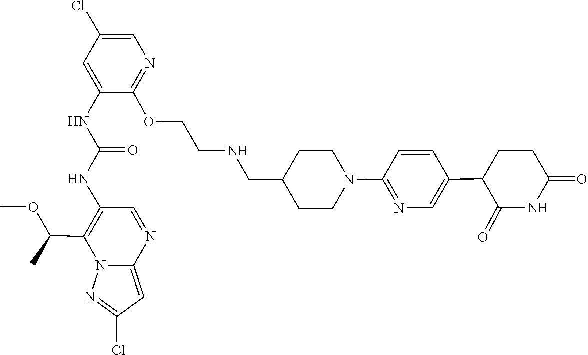 [SMILES: CO[C@H](C)c1c(NC(=O)Nc2cc(Cl)cnc2OCCNCC2CCN(c3ccc(C4CCC(=O)NC4=O)cn3)CC2)cnc2cc(Cl)nn12] | MALT1 |
| 328 | 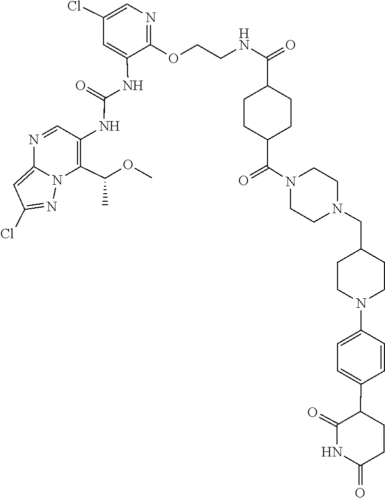 [SMILES: CO[C@H](C)c1c(NC(=O)Nc2cc(Cl)cnc2OCCNC(=O)C2CCC(C(=O)N3CCN(CC4CCN(c5ccc(C6CCC(=O)NC6=O)cc5)CC4)CC3)CC2)cnc2cc(Cl)nn12] | MALT1 |
| 329 | 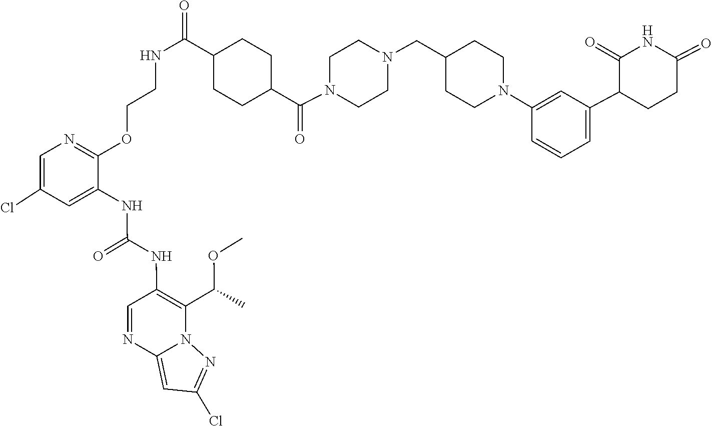 [SMILES: CO[C@H](C)c1c(NC(=O)Nc2cc(Cl)cnc2OCCNC(=O)C2CCC(C(=O)N3CCN(CC4CCN(c5cccc(C6CCC(=O)NC6=O)c5)CC4)CC3)CC2)cnc2cc(Cl)nn12] | MALT1 |
| 330 | 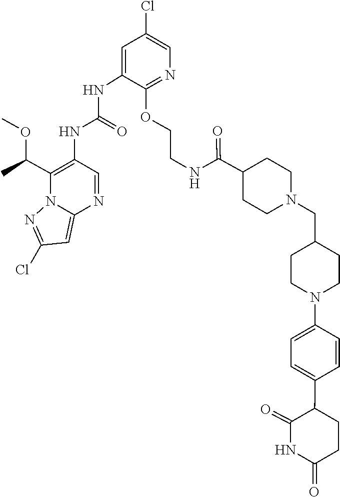 [SMILES: CO[C@H](C)c1c(NC(=O)Nc2cc(Cl)cnc2OCCNC(=O)C2CCN(CC3CCN(c4ccc(C5CCC(=O)NC5=O)cc4)CC3)CC2)cnc2cc(Cl)nn12] | MALT1 |
| 331 | 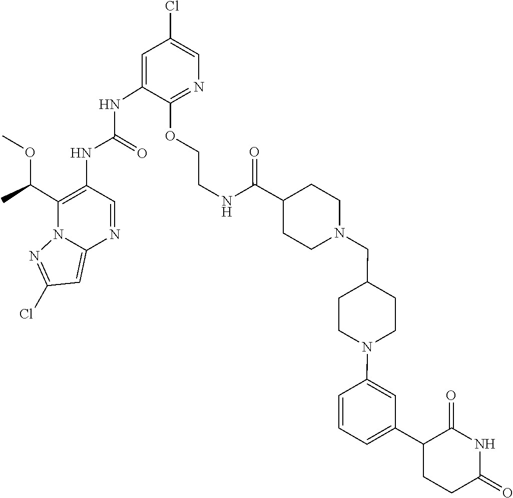 [SMILES: CO[C@H](C)c1c(NC(=O)Nc2cc(Cl)cnc2OCCNC(=O)C2CCN(CC3CCN(c4cccc(C5CCC(=O)NC5=O)c4)CC3)CC2)cnc2cc(Cl)nn12] | MALT1 |
| 332 | 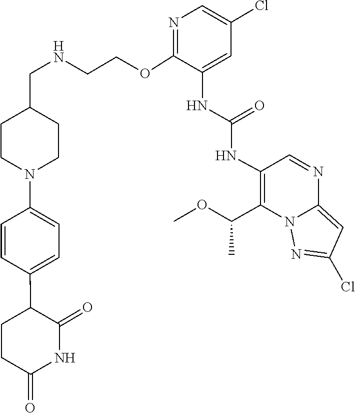 [SMILES: CO[C@@H](C)c1c(NC(=O)Nc2cc(Cl)cnc2OCCNCC2CCN(c3ccc(C4CCC(=O)NC4=O)cc3)CC2)cnc2cc(Cl)nn12] | MALT1 |
| 333 | 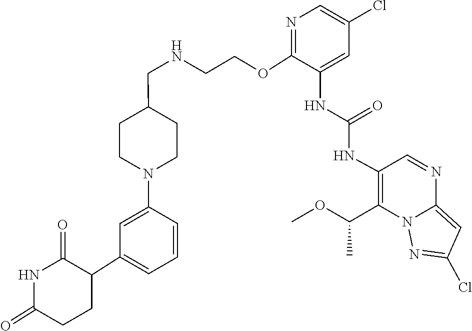 [SMILES: CO[C@@H](C)c1c(NC(=O)Nc2cc(Cl)cnc2OCCNCC2CCN(c3cccc(C4CCC(=O)NC4=O)c3)CC2)cnc2cc(Cl)nn12] | MALT1 |
| 334 | 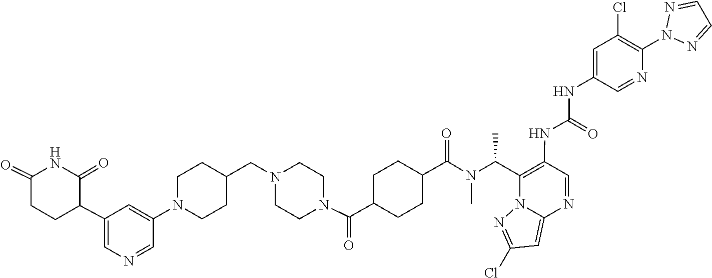 | MALT1 |
| 335 | 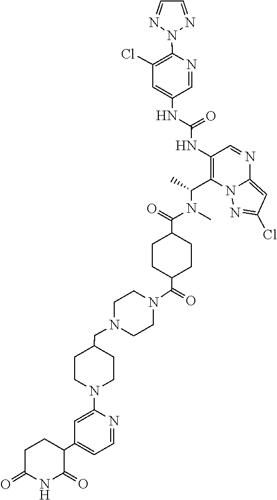 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4cc(C5CCC(=O)NC5=O)ccn4)CC3)CC2)CC1] | MALT1 |
| 336 | 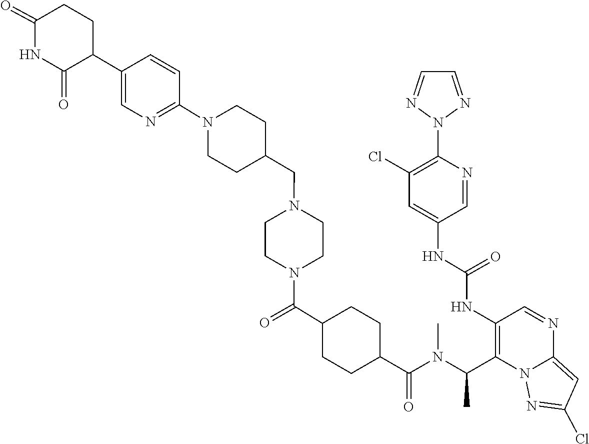 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4ccc(C5CCC(=O)NC5=O)cn4)CC3)CC2)CC1] | MALT1 |
| 337 | 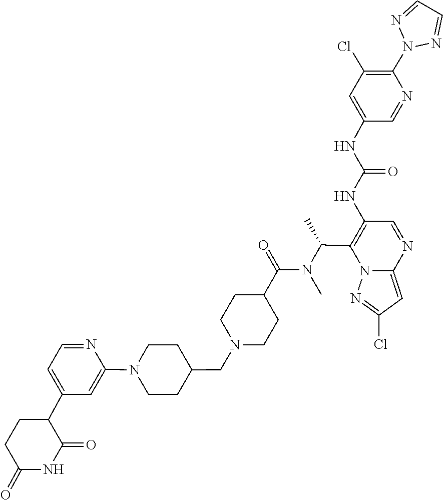 [SMILES: C[C@H](c1c(NC(=O)Nc2cnc(-n3nccn3)c(Cl)c2)cnc2cc(Cl)nn12)N(C)C(=O)C1CCN(CC2CCN(c3cc(C4CCC(=O)NC4=O)ccn3)CC2)CC1] | MALT1 |
| 338 | 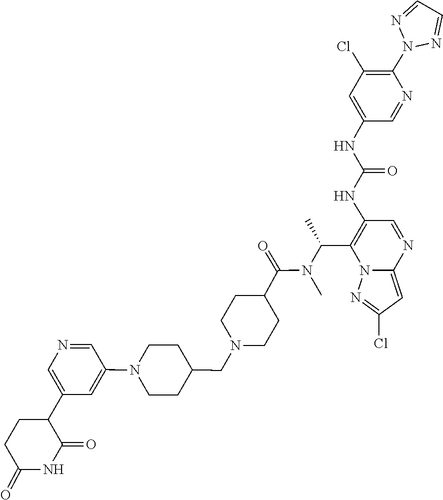 [SMILES: C[C@H](c1c(NC(=O)Nc2cnc(-n3nccn3)c(Cl)c2)cnc2cc(Cl)nn12)N(C)C(=O)C1CCN(CC2CCN(c3cncc(C4CCC(=O)NC4=O)c3)CC2)CC1] | MALT1 |
| 339 | 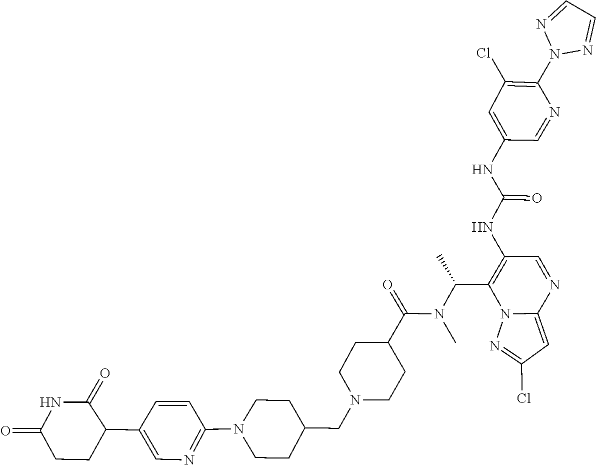 [SMILES: C[C@H](c1c(NC(=O)Nc2cnc(-n3nccn3)c(Cl)c2)cnc2cc(Cl)nn12)N(C)C(=O)C1CCN(CC2CCN(c3ccc(C4CCC(=O)NC4=O)cn3)CC2)CC1] | MALT1 |
| 340 | 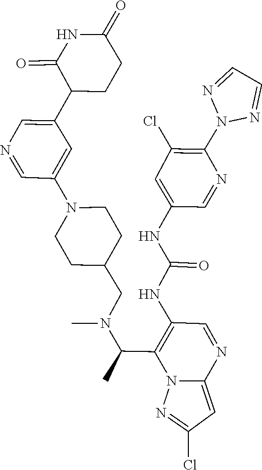 [SMILES: C[C@H](c1c(NC(=O)Nc2cnc(-n3nccn3)c(Cl)c2)cnc2cc(Cl)nn12)N(C)CC1CCN(c2cncc(C3CCC(=O)NC3=O)c2)CC1] | MALT1 |
| 341 | 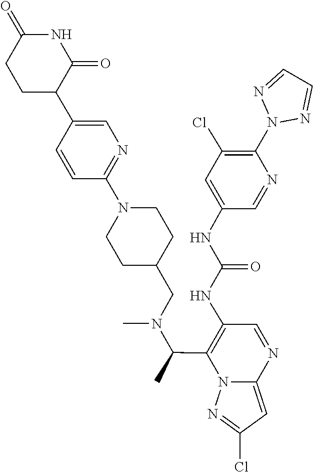 [SMILES: C[C@H](c1c(NC(=O)Nc2cnc(-n3nccn3)c(Cl)c2)cnc2cc(Cl)nn12)N(C)CC1CCN(c2ccc(C3CCC(=O)NC3=O)cn2)CC1] | MALT1 |
| 342 | 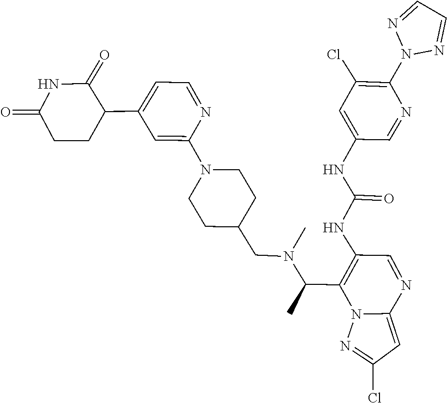 | MALT1 |
| 343 | 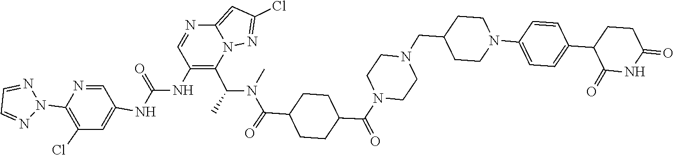 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4ccc(C5CCC(=O)NC5=O)cc4)CC3)CC2)CC1] | MALT1 |
| 344 | 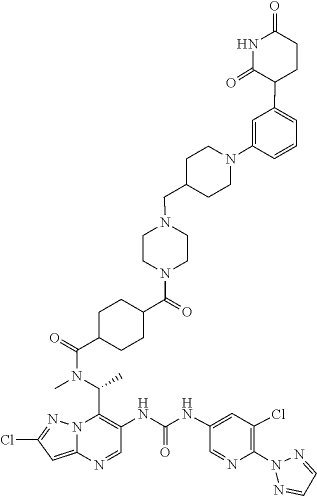 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4cccc(C5CCC(=O)NC5=O)c4)CC3)CC2)CC1] | MALT1 |
| 345 | 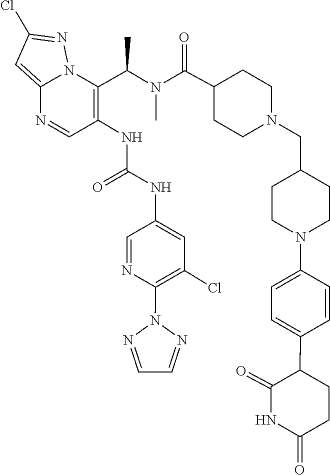 [SMILES: C[C@H](c1c(NC(=O)Nc2cnc(-n3nccn3)c(Cl)c2)cnc2cc(Cl)nn12)N(C)C(=O)C1CCN(CC2CCN(c3ccc(C4CCC(=O)NC4=O)cc3)CC2)CC1] | MALT1 |
| 346 | 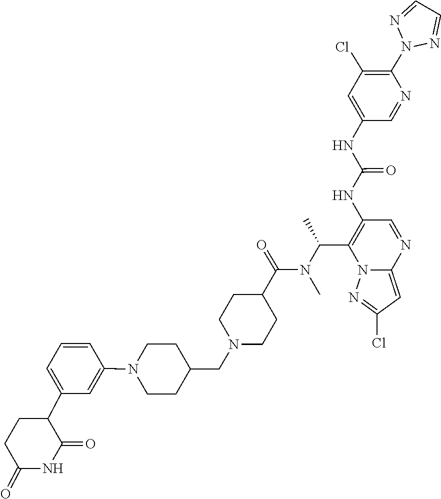 [SMILES: C[C@H](c1c(NC(=O)Nc2cnc(-n3nccn3)c(Cl)c2)cnc2cc(Cl)nn12)N(C)C(=O)C1CCN(CC2CCN(c3cccc(C4CCC(=O)NC4=O)c3)CC2)CC1] | MALT1 |
| 347 | 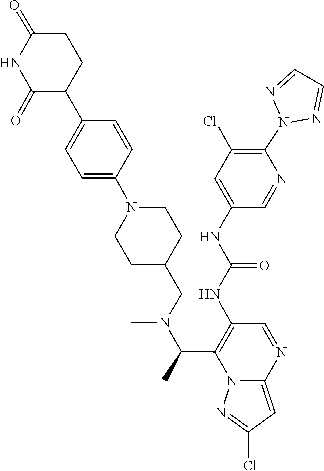 [SMILES: C[C@H](c1c(NC(=O)Nc2cnc(-n3nccn3)c(Cl)c2)cnc2cc(Cl)nn12)N(C)CC1CCN(c2ccc(C3CCC(=O)NC3=O)cc2)CC1] | MALT1 |
| 348 | 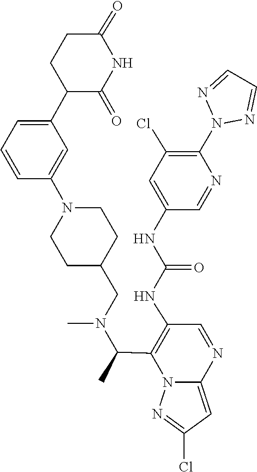 [SMILES: C[C@H](c1c(NC(=O)Nc2cnc(-n3nccn3)c(Cl)c2)cnc2cc(Cl)nn12)N(C)CC1CCN(c2cccc(C3CCC(=O)NC3=O)c2)CC1] | MALT1 |
| 349 | 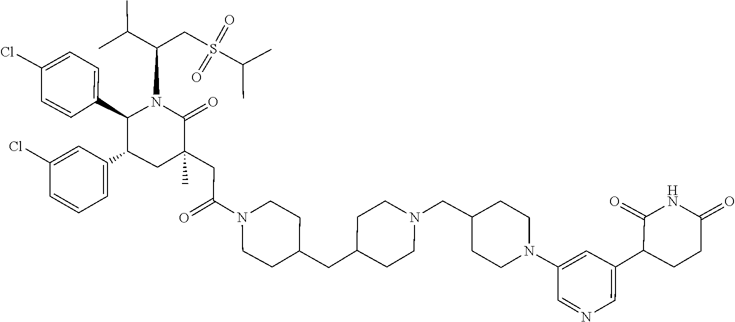 [SMILES: CC(C)[C@@H](CS(=O)(=O)C(C)C)N1C(=O)[C@@](C)(CC(=O)N2CCC(CC3CCN(CC4CCN(c5cncc(C6CCC(=O)NC6=O)c5)CC4)CC3)CC2)C[C@H](c2cccc(Cl)c2)[C@H]1c1ccc(Cl)cc1] | CDK4/6 |
| 350 | 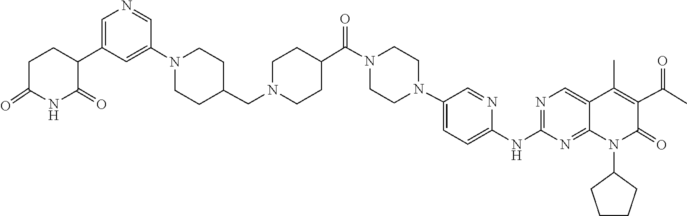 [SMILES: CC(=O)c1c(C)c2cnc(Nc3ccc(N4CCN(C(=O)C5CCN(CC6CCN(c7cncc(C8CCC(=O)NC8=O)c7)CC6)CC5)CC4)cn3)nc2n(C2CCCC2)c1=O] | CDK4/6 |
| 351 | 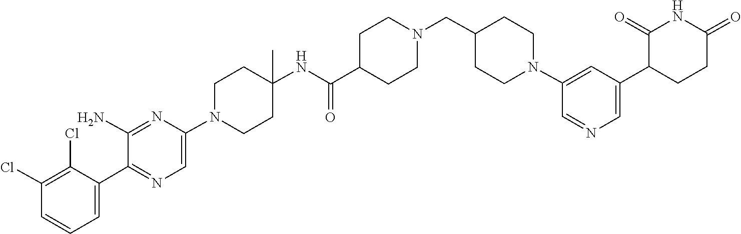 [SMILES: CC1(NC(=O)C2CCN(CC3CCN(c4cncc(C5CCC(=O)NC5=O)c4)CC3)CC2)CCN(c2cnc(-c3cccc(Cl)c3Cl)c(N)n2)CC1] | SHP-2 |
| 352 | 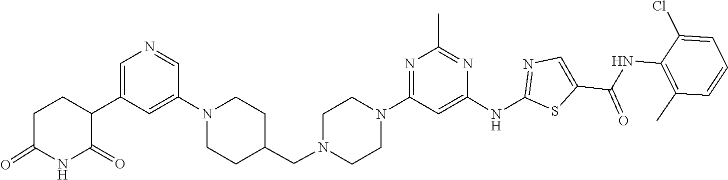 [SMILES: Cc1nc(Nc2ncc(C(=O)Nc3c(C)cccc3Cl)s2)cc(N2CCN(CC3CCN(c4cncc(C5CCC(=O)NC5=O)c4)CC3)CC2)n1] | BCR- ABL |
| 353 | 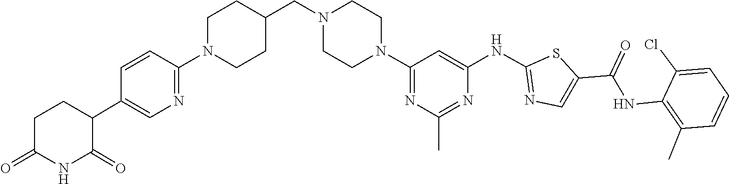 [SMILES: Cc1nc(Nc2ncc(C(=O)Nc3c(C)cccc3Cl)s2)cc(N2CCN(CC3CCN(c4ccc(C5CCC(=O)NC5=O)cn4)CC3)CC2)n1] | BCR- ABL |
| 354 | 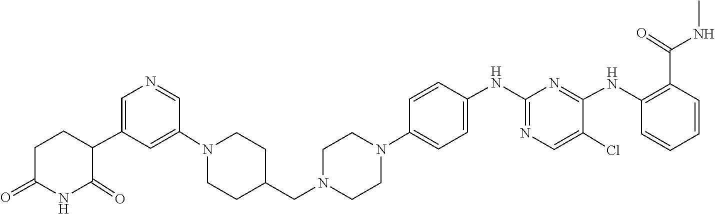 [SMILES: CNC(=O)c1ccccc1Nc1nc(Nc2ccc(N3CCN(CC4CCN(c5cncc(C6CCC(=O)NC6=O)c5)CC4)CC3)cc2)ncc1Cl] | Kinase |
| 355 | 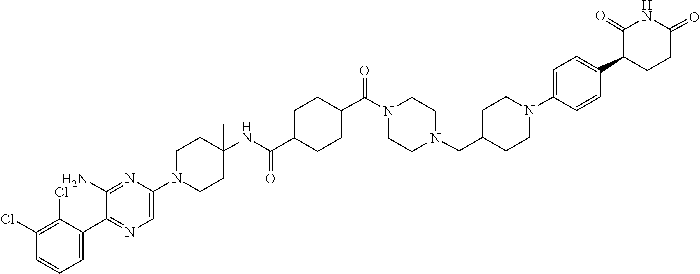 [SMILES: CC1(NC(=O)C2CCC(C(=O)N3CCN(CC4CCN(c5ccc([C@@H]6CCC(=O)NC6=O)cc5)CC4)CC3)CC2)CCN(c2cnc(-c3cccc(Cl)c3Cl)c(N)n2)CC1] | SHP |
| 356 | 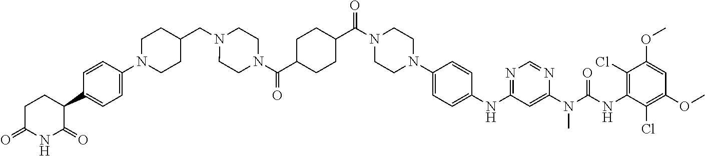 [SMILES: COc1cc(OC)c(Cl)c(NC(=O)N(C)c2cc(Nc3ccc(N4CCN(C(=O)C5CCC(C(=O)N6CCN(CC7CCN(c8ccc([C@@H]9CCC(=O)NC9=O)cc8)CC7)CC6)CC5)CC4)cc3)ncn2)c1Cl] | FGFR1/3 |
| 357 | 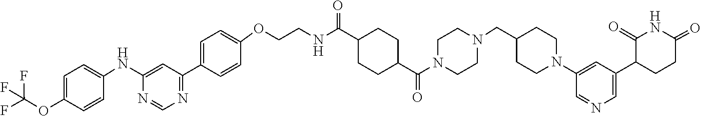 [SMILES: O=C1CCC(c2cncc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] | BCR- ABL |
| 358 | 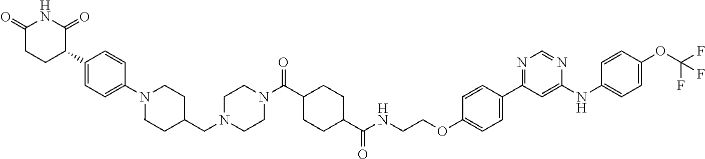 [SMILES: O=C1CC[C@@H](c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)cc2)C(=O)N1] | BCR- ABL |
| 359 | 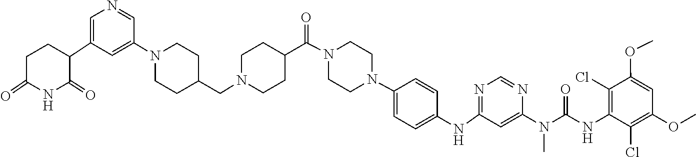 [SMILES: COc1cc(OC)c(Cl)c(NC(=O)N(C)c2cc(Nc3ccc(N4CCN(C(=O)C5CCN(CC6CCN(c7cncc(C8CCC(=O)NC8=O)c7)CC6)CC5)CC4)cc3)ncn2)c1Cl] | FGFR1/3 |
| 360 | 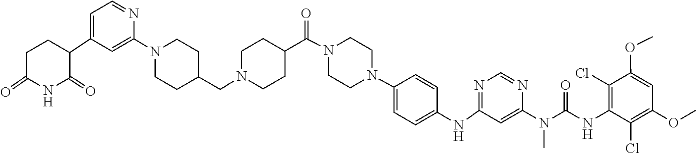 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] | FGFR1/3 |
| 361 | 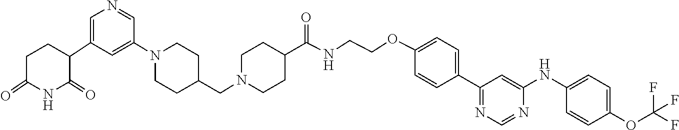 [SMILES: O=C1CCC(c2cncc(N3CCC(CN4CCC(C(=O)NCCOc5ccc(-c6cc(Nc7ccc(OC(F)(F)F)cc7)ncn6)cc5)CC4)CC3)c2)C(=O)N1] | BCR- ABL |
| 362 | 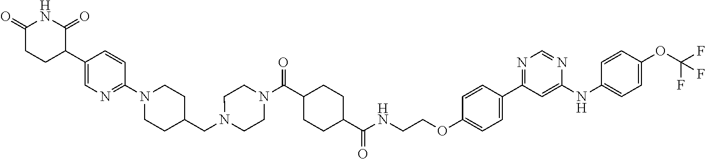 [SMILES: O=C1CCC(c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)nc2)C(=O)N1] | BCR- ABL |
| 363 | 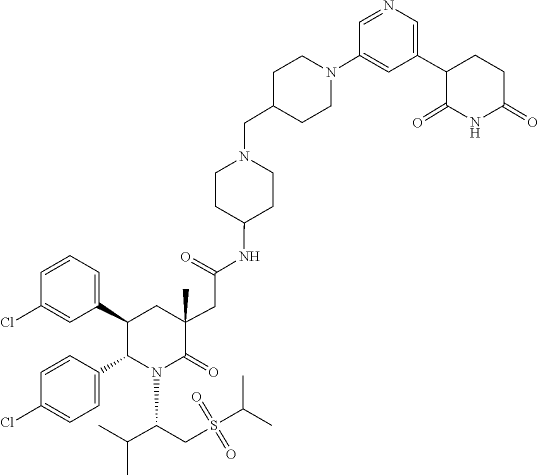 [SMILES: CC(C)[C@@H](CS(=O)(=O)C(C)C)N1C(=O)[C@@](C)(CC(=O)NC2CCN(CC3CCN(c4cncc(C5CCC(=O)NC5=O)c4)CC3)CC2)C[C@H](c2cccc(Cl)c2)[C@H]1c1ccc(Cl)cc1] | MDM2 |
| 364 | 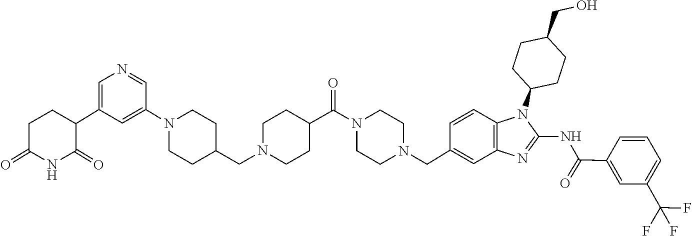 [SMILES: O=C1CCC(c2cncc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)c2)C(=O)N1] | IRAK1 |
| 365 | 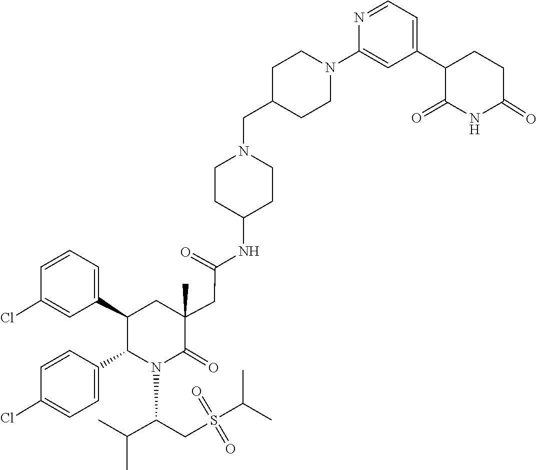 [SMILES: CC(C)[C@@H](CS(=O)(=O)C(C)C)N1C(=O)[C@@](C)(CC(=O)NC2CCN(CC3CCN(c4cc(C5CCC(=O)NC5=O)ccn4)CC3)CC2)C[C@H](c2cccc(Cl)c2)[C@H]1c1ccc(Cl)cc1] | MDM12 |
| 366 | 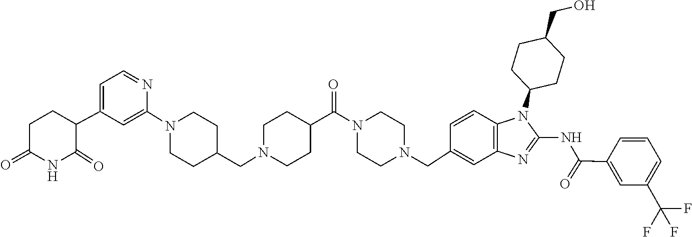 [SMILES: O=C1CCC(c2ccnc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)c2)C(=O)N1] | IRAK1 |
| 367 | 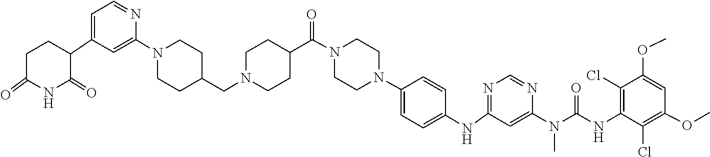 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] | FGFR1/3 |
| 368 | 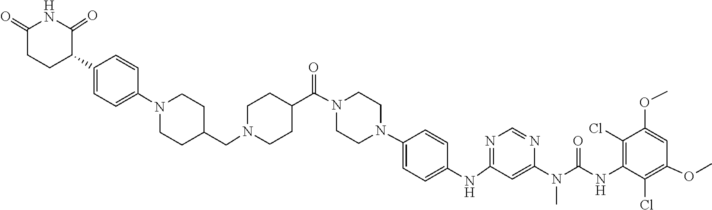 [SMILES: COc1cc(OC)c(Cl)c(NC(=O)N(C)c2cc(Nc3ccc(N4CCN(C(=O)C5CCN(CC6CCN(c7ccc([C@@H]8CCC(=O)NC8=O)cc7)CC6)CC5)CC4)cc3)ncn2)c1Cl] | FGFR1/3 |
| 369 | 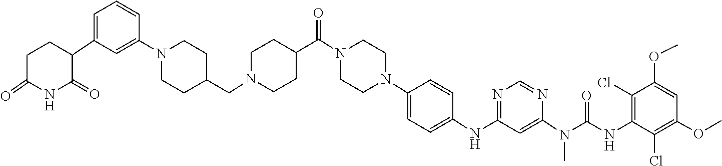 [SMILES: COc1cc(OC)c(Cl)c(NC(=O)N(C)c2cc(Nc3ccc(N4CCN(C(=O)C5CCN(CC6CCN(c7cccc(C8CCC(=O)NC8=O)c7)CC6)CC5)CC4)cc3)ncn2)c1Cl] | FGFR1/3 |
| 370 | 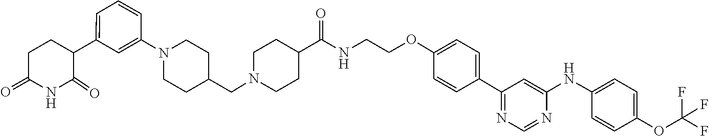 [SMILES: O=C1CCC(c2cccc(N3CCC(CN4CCC(C(=O)NCCOc5ccc(-c6cc(Nc7ccc(OC(F)(F)F)cc7)ncn6)cc5)CC4)CC3)c2)C(=O)N1] | BCR- ABL |
| 371 | 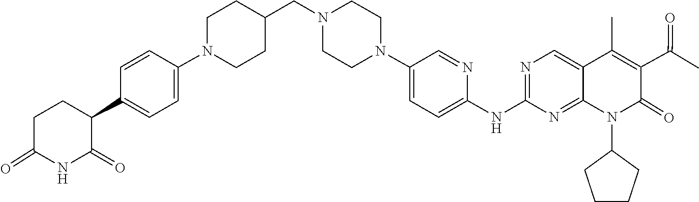 [SMILES: CCN(CCC(C)CN1CCN(c2ccc(Nc3ncc4c(C)c(C(C)=O)c(=O)n(C5CCCC5)c4n3)nc2)CC1)c1ccc([C@@H]2CCC(=O)NC2=O)cc1] | SHP-2 |
| 372 | 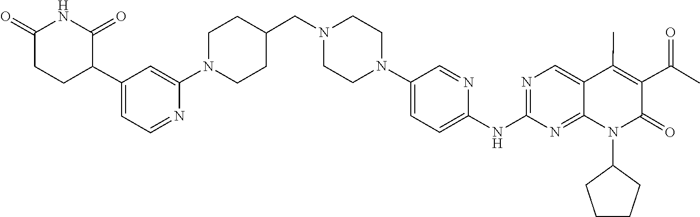 [SMILES: CC(=O)c1c(C)c2cnc(Nc3ccc(N4CCN(CC5CCN(c6cc(C7CCC(=O)NC7=O)ccn6)CC5)CC4)cn3)nc2n(C2CCCC2)c1=O] | CDK4/6 |
| 373 | 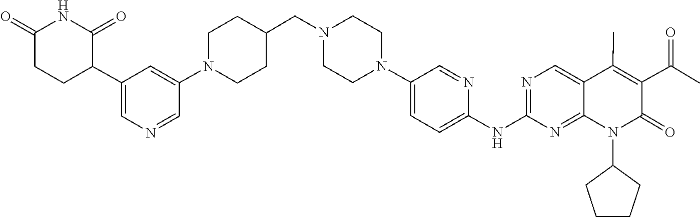 [SMILES: CC(=O)c1c(C)c2cnc(Nc3ccc(N4CCN(CC5CCN(c6cncc(C7CCC(=O)NC7=O)c6)CC5)CC4)cn3)nc2n(C2CCCC2)c1=O] | CDK4/6 |
| 374 | 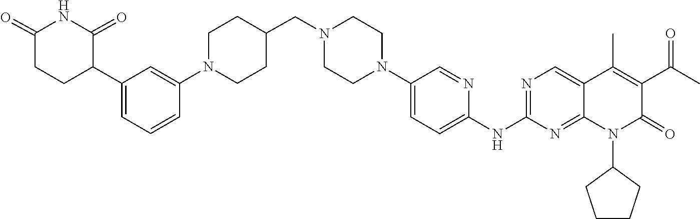 [SMILES: CC(=O)c1c(C)c2cnc(Nc3ccc(N4CCN(CC5CCN(c6cccc(C7CCC(=O)NC7=O)c6)CC5)CC4)cn3)nc2n(C2CCCC2)c1=O] | FGFR1/3 |
| 375 | 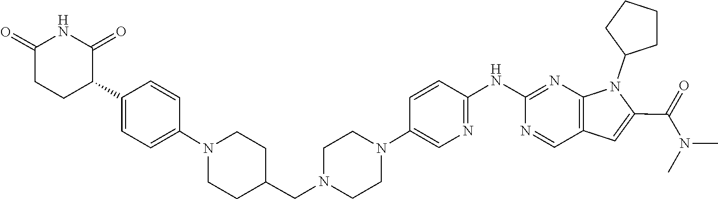 [SMILES: CN(C)C(=O)c1cc2cnc(Nc3ccc(N4CCN(CC5CCN(c6ccc([C@@H]7CCC(=O)NC7=O)cc6)CC5)CC4)cn3)nc2n1C1CCCC1] | SHP-2 |
| 376 | 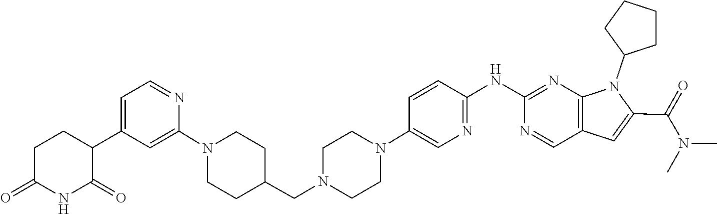 [SMILES: CN(C)C(=O)c1cc2cnc(Nc3ccc(N4CCN(CC5CCN(c6cc(C7CCC(=O)NC7=O)ccn6)CC5)CC4)cn3)nc2n1C1CCCC1] | CDK4/6 |
| 377 | 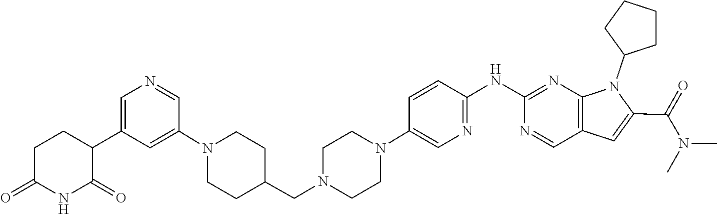 [SMILES: CN(C)C(=O)c1cc2cnc(Nc3ccc(N4CCN(CC5CCN(c6cncc(C7CCC(=O)NC7=O)c6)CC5)CC4)cn3)nc2n1C1CCCC1] | CDK4/6 |
| 378 | 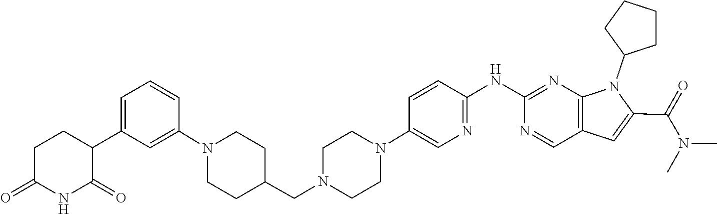 [SMILES: CN(C)C(=O)c1cc2cnc(Nc3ccc(N4CCN(CC5CCN(c6cccc(C7CCC(=O)NC7=O)c6)CC5)CC4)cn3)nc2n1C1CCCC1] | CDK4/6 |
| 379 | 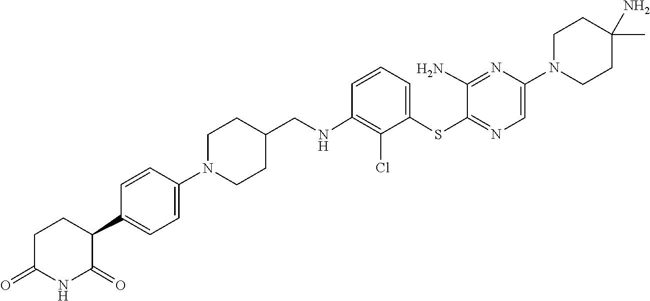 [SMILES: CC1(N)CCN(c2cnc(Sc3cccc(NCC4CCN(c5ccc([C@@H]6CCC(=O)NC6=O)cc5)CC4)c3Cl)c(N)n2)CC1] | SHP-2 |
| 380 | 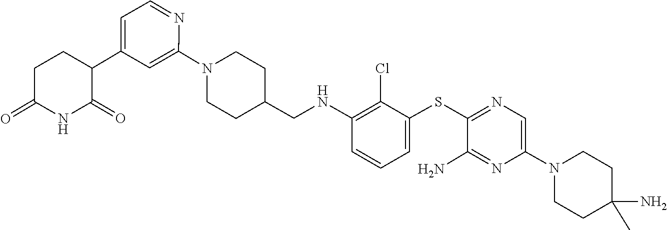 [SMILES: CC1(N)CCN(c2cnc(Sc3cccc(NCC4CCN(c5cc(C6CCC(=O)NC6=O)ccn5)CC4)c3Cl)c(N)n2)CC1] | SHP-2 |
| 381 | 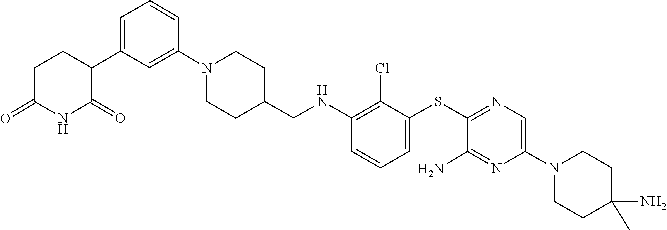 [SMILES: CC1(N)CCN(c2cnc(Sc3cccc(NCC4CCN(c5cccc(C6CCC(=O)NC6=O)c5)CC4)c3Cl)c(N)n2)CC1] | SHP-2 |
| 382 | 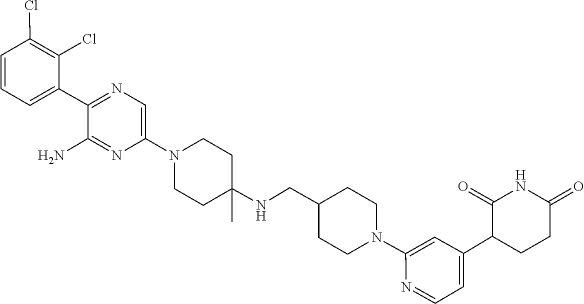 [SMILES: CC1(NCC2CCN(c3cc(C4CCC(=O)NC4=O)ccn3)CC2)CCN(c2cnc(-c3cccc(Cl)c3Cl)c(N)n2)CC1] | SHP-2 |
| 383 | 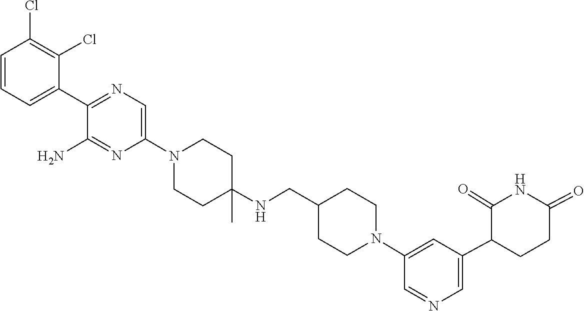 [SMILES: CC1(NCC2CCN(c3cncc(C4CCC(=O)NC4=O)c3)CC2)CCN(c2cnc(-c3cccc(Cl)c3Cl)c(N)n2)CC1] | SHP-2 |
| 384 | 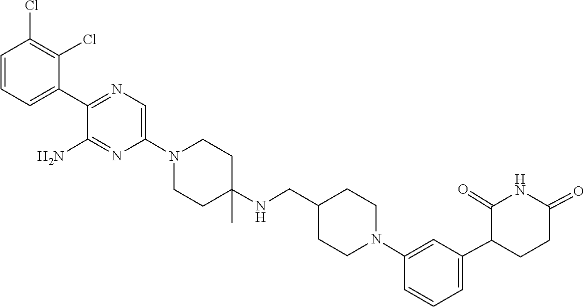 [SMILES: CC1(NCC2CCN(c3cccc(C4CCC(=O)NC4=O)c3)CC2)CCN(c2cnc(-c3cccc(Cl)c3Cl)c(N)n2)CC1] | SHP-2 |
| 385 | 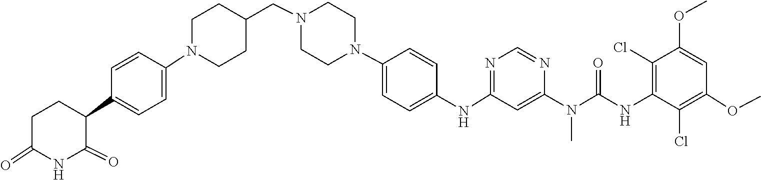 [SMILES: COc1cc(OC)c(Cl)c(NC(=O)N(C)c2cc(Nc3ccc(N4CCN(CC5CCN(c6ccc([C@@H]7CCC(=O)NC7=O)cc6)CC5)CC4)cc3)ncn2)c1Cl] | FGFR1/3 |
| 386 | 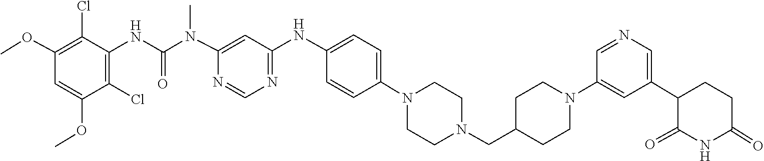 [SMILES: COc1cc(OC)c(Cl)c(NC(=O)N(C)c2cc(Nc3ccc(N4CCN(CC5CCN(c6cncc(C7CCC(=O)NC7=O)c6)CC5)CC4)cc3)ncn2)c1Cl] | FGFR1/3 |
| 387 | 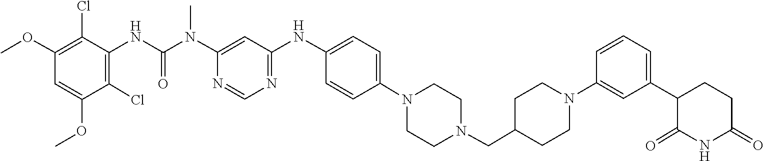 [SMILES: COc1cc(OC)c(Cl)c(NC(=O)N(C)c2cc(Nc3ccc(N4CCN(CC5CCN(c6cccc(C7CCC(=O)NC7=O)c6)CC5)CC4)cc3)ncn2)c1Cl] | FGFR1/3 |
| 388 | 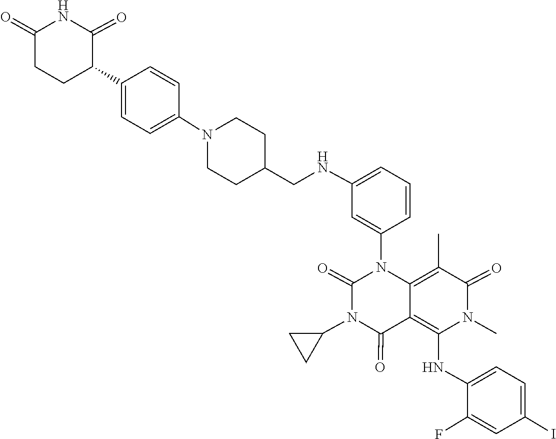 [SMILES: Cc1c(=O)n(C)c(Nc2ccc(I)cc2F)c2c(=O)n(C3CC3)c(=O)n(-c3cccc(NCC4CCN(c5ccc([C@@H]6CCC(=O)NC6=O)cc5)CC4)c3)c12] | MEK |
| 389 | 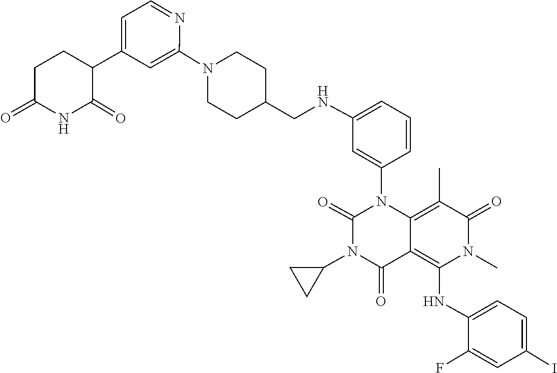 [SMILES: Cc1c(=O)n(C)c(Nc2ccc(I)cc2F)c2c(=O)n(C3CC3)c(=O)n(-c3cccc(NCC4CCN(c5cc(C6CCC(=O)NC6=O)ccn5)CC4)c3)c12] | MEK |
| 390 | 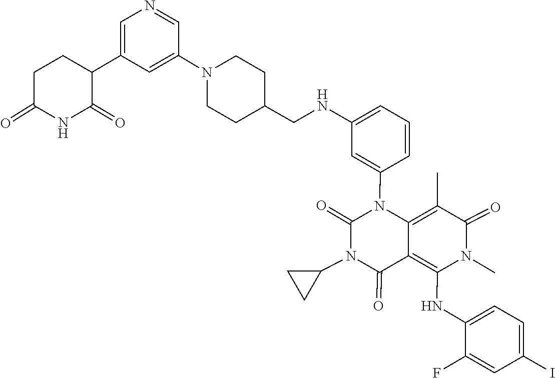 [SMILES: Cc1c(=O)n(C)c(Nc2ccc(I)cc2F)c2c(=O)n(C3CC3)c(=O)n(-c3cccc(NCC4CCN(c5cncc(C6CCC(=O)NC6=O)c5)CC4)c3)c12] | MEK |
| 391 | 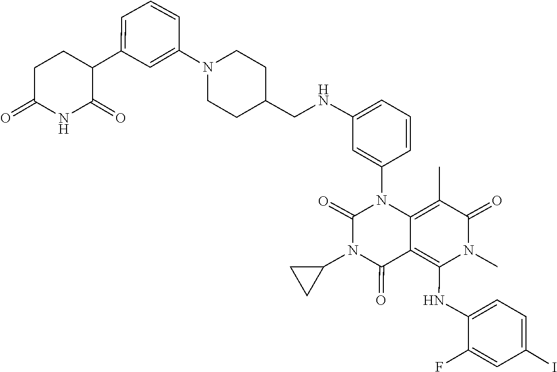 [SMILES: Cc1c(=O)n(C)c(Nc2ccc(I)cc2F)c2c(=O)n(C3CC3)c(=O)n(-c3cccc(NCC4CCN(c5cccc(C6CCC(=O)NC6=O)c5)CC4)c3)c12] | MEK |
| 392 | 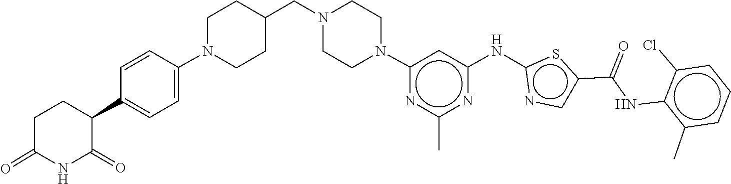 | BCR- ABL |
| 393 | 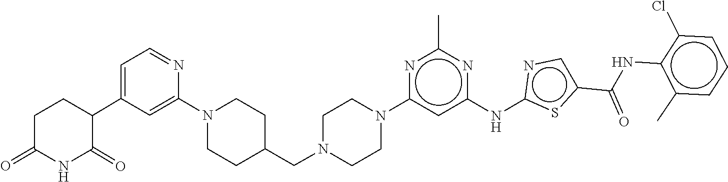 | BCR- ABL |
| 394 | 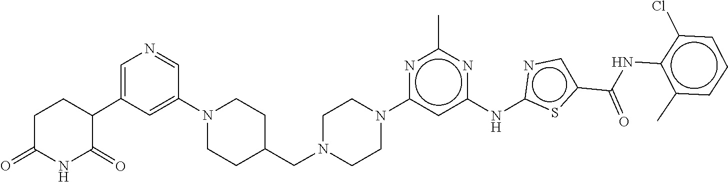 | BCR- ABL |
| 395 | 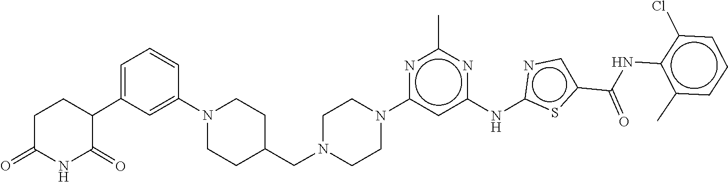 | BCR- ABL |
| 396 | 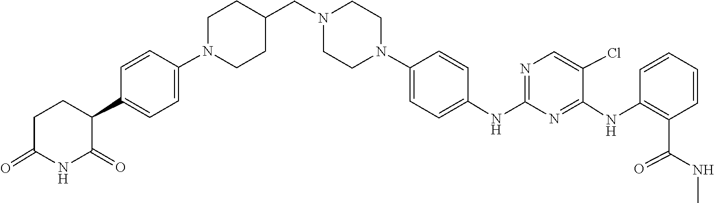 [SMILES: CNC(=O)c1ccccc1Nc1nc(Nc2ccc(N3CCN(CC4CCN(c5ccc([C@@H]6CCC(=O)NC6=O)cc5)CC4)CC3)cc2)ncc1Cl] | Pan kinase |
| 397 | 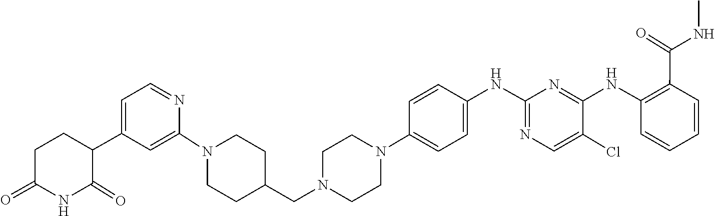 [SMILES: CNC(=O)c1ccccc1Nc1nc(Nc2ccc(N3CCN(CC4CCN(c5cc(C6CCC(=O)NC6=O)ccn5)CC4)CC3)cc2)ncc1Cl] | Pan kinase |
| 398 | 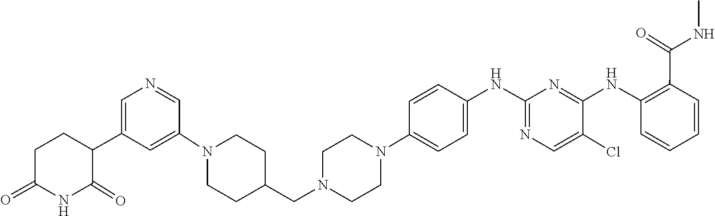 [SMILES: CNC(=O)c1ccccc1Nc1nc(Nc2ccc(N3CCN(CC4CCN(c5cncc(C6CCC(=O)NC6=O)c5)CC4)CC3)cc2)ncc1Cl] | Pan kinase |
| 399 | 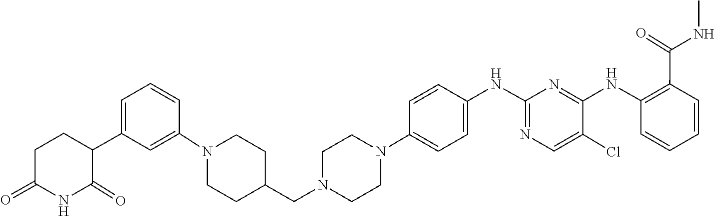 [SMILES: CNC(=O)c1ccccc1Nc1nc(Nc2ccc(N3CCN(CC4CCN(c5cccc(C6CCC(=O)NC6=O)c5)CC4)CC3)cc2)ncc1Cl] | Pan kinase |
| 400 | 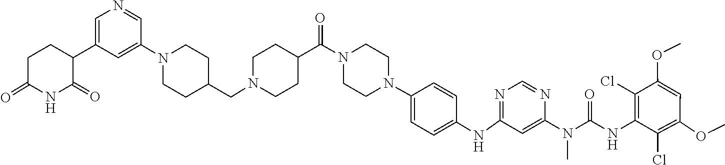 [SMILES: COc1cc(OC)c(Cl)c(NC(=O)N(C)c2cc(Nc3ccc(N4CCN(C(=O)C5CCN(CC6CCN(c7cncc(C8CCC(=O)NC8=O)c7)CC6)CC5)CC4)cc3)ncn2)c1Cl] | FGFR1/3 |
| 401 | 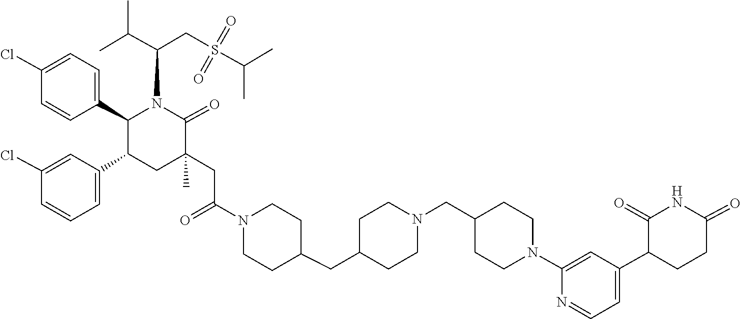 [SMILES: CC(C)[C@@H](CS(=O)(=O)C(C)C)N1C(=O)[C@@](C)(CC(=O)N2CCC(CC3CCN(CC4CCN(c5cc(C6CCC(=O)NC6=O)ccn5)CC4)CC3)CC2)C[C@H](c2cccc(Cl)c2)[C@H]1c1ccc(Cl)cc1] | MDM2 |
| 402 | 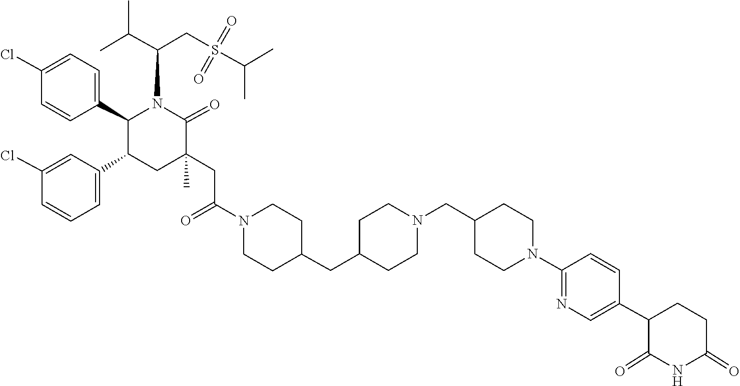 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cn5)CC4)CC3)CC2)C[C@H](c2cccc(Cl)c2)[C@H]1c1ccc(Cl)cc1] | MDM2 |
| 403 | 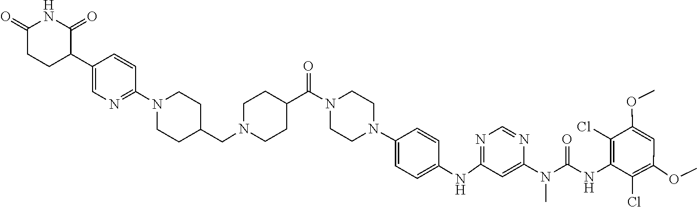 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cn7)CC6)CC5)CC4)cc3)ncn2)c1Cl] | FGFR1/3 |
| 404 | 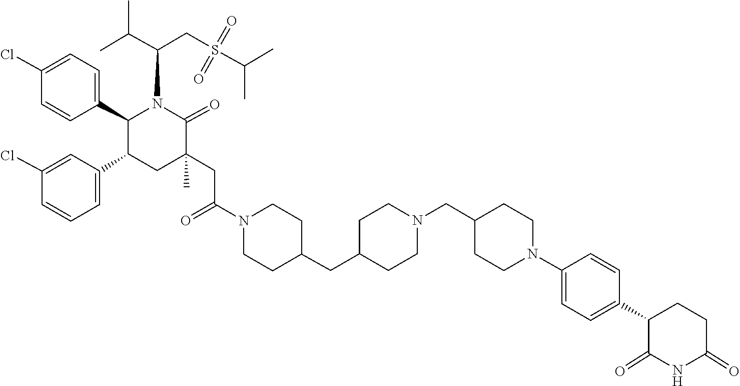 [SMILES: CC(C)[C@@H](CS(=O)(=O)C(C)C)N1C(=O)[C@@](C)(CC(=O)N2CCC(CC3CCN(CC4CCN(c5ccc([C@@H]6CCC(=O)NC6=O)cc5)CC4)CC3)CC2)C[C@H](c2cccc(Cl)c2)[C@H]1c1ccc(Cl)cc1] | MDM2 |
| 405 | 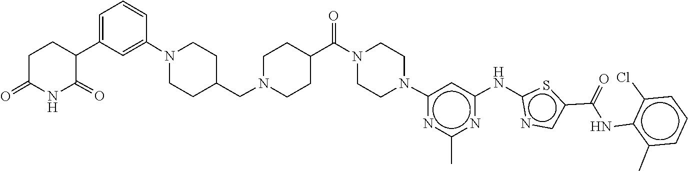 | BCR- ABL |
| 406 | 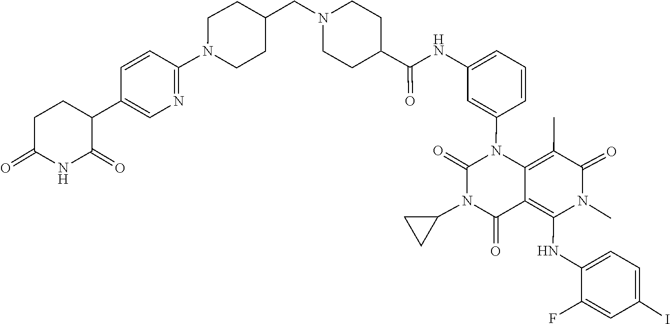 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] | MEK |
| 407 | 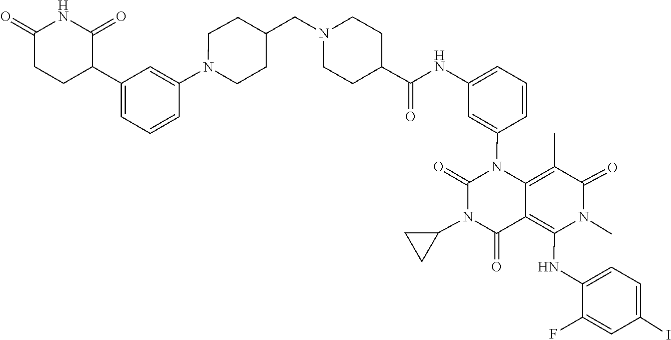 [SMILES: Cc1c(=O)n(C)c(Nc2ccc(I)cc2F)c2c(=O)n(C3CC3)c(=O)n(-c3cccc(NC(=O)C4CCN(CC5CCN(c6cccc(C7CCC(=O)NC7=O)c6)CC5)CC4)c3)c12] | MEK |
| 408 | 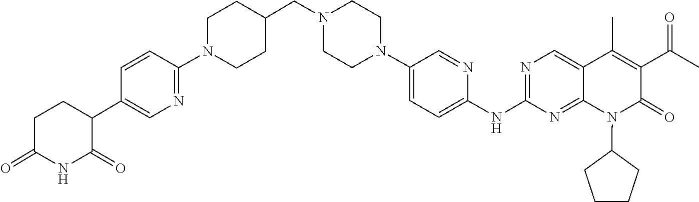 [SMILES: CC(=O)c1c(C)c2cnc(Nc3ccc(N4CCN(CC5CCN(c6ccc(C7CCC(=O)NC7=O)cn6)CC5)CC4)cn3)nc2n(C2CCCC2)c1=O] | CDK4/6 |
| 409 | 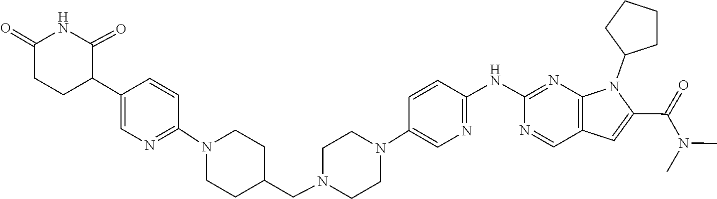 [SMILES: CN(C)C(=O)c1cc2cnc(Nc3ccc(N4CCN(CC5CCN(c6ccc(C7CCC(=O)NC7=O)cn6)CC5)CC4)cn3)nc2n1C1CCCC1] | CDK4/6 |
| 410 | 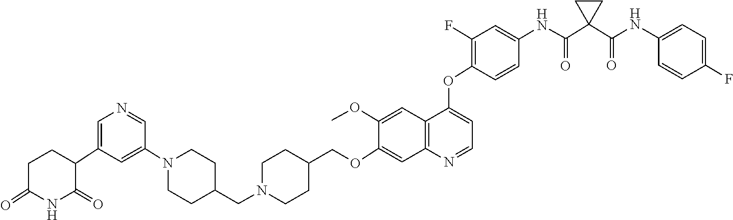 [SMILES: COc1cc2c(Oc3ccc(NC(=O)C4(C(=O)Nc5ccc(F)cc5)CC4)cc3F)ccnc2cc1OCC1CCN(CC2CCN(c3cncc(C4CCC(=O)NC4=O)c3)CC2)CC1] | Pan kinase |
| 411 | 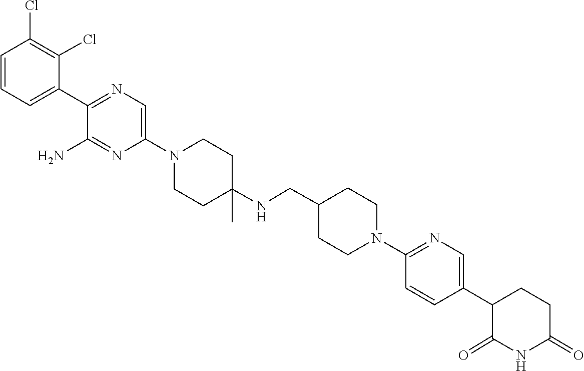 [SMILES: CC1(NCC2CCN(c3ccc(C4CCC(=O)NC4=O)cn3)CC2)CCN(c2cnc(-c3cccc(Cl)c3Cl)c(N)n2)CC1] | SHP-2 |
| 412 | 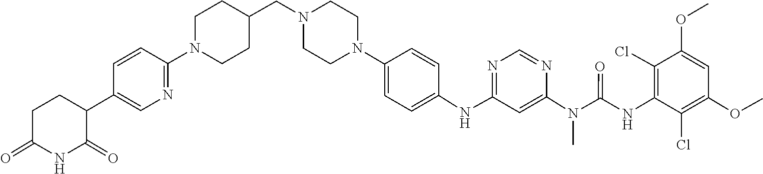 [SMILES: COc1cc(OC)c(Cl)c(NC(=O)N(C)c2cc(Nc3ccc(N4CCN(CC5CCN(c6ccc(C7CCC(=O)NC7=O)cn6)CC5)CC4)cc3)ncn2)c1Cl] | FGFR1/3 |
| 413 | 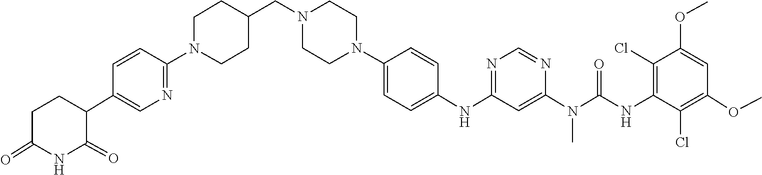 [SMILES: COc1cc(OC)c(Cl)c(NC(=O)N(C)c2cc(Nc3ccc(N4CCN(CC5CCN(c6ccc(C7CCC(=O)NC7=O)cn6)CC5)CC4)cc3)ncn2)c1Cl] | FGFR1/3 |
| 414 | 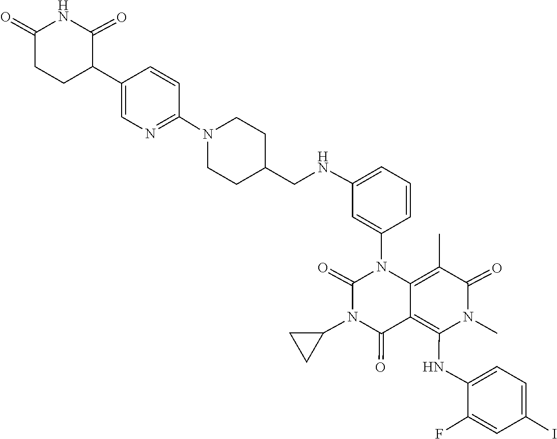 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cn5)CC4)c3)c12] | MEK |
| 415 | 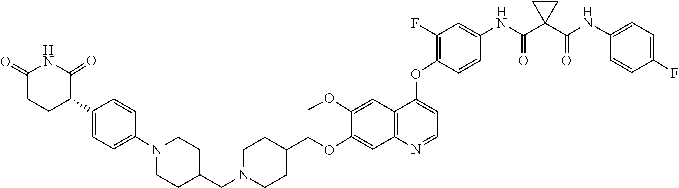 [SMILES: COc1cc2c(Oc3ccc(NC(=O)C4(C(=O)Nc5ccc(F)cc5)CC4)cc3F)ccnc2cc1OCC1CCN(CC2CCN(c3ccc([C@@H]4CCC(=O)NC4=O)cc3)CC2)CC1] | Pan kinase |
| 416 | 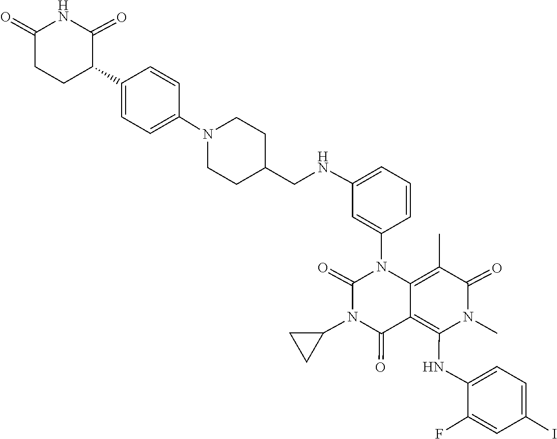 [SMILES: Cc1c(=O)n(C)c(Nc2ccc(I)cc2F)c2c(=O)n(C3CC3)c(=O)n(-c3cccc(NCC4CCN(c5ccc([C@@H]6CCC(=O)NC6=O)cc5)CC4)c3)c12] | MEK |
| 417 | 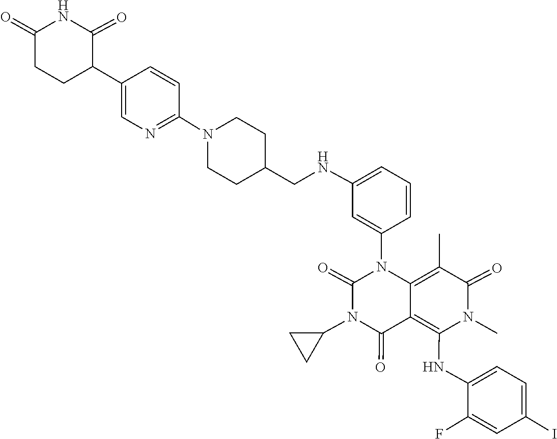 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cn5)CC4)c3)c12] | MEK |
| 418 | 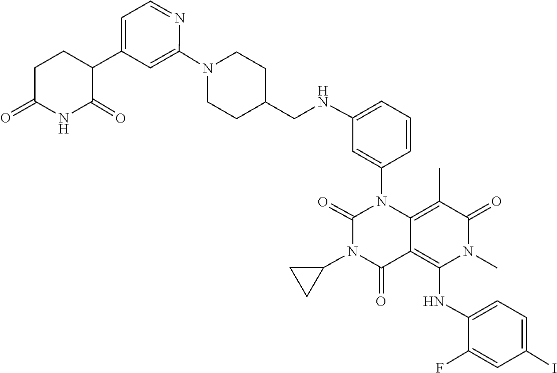 [SMILES: Cc1c(=O)n(C)c(Nc2ccc(I)cc2F)c2c(=O)n(C3CC3)c(=O)n(-c3cccc(NCC4CCN(c5cc(C6CCC(=O)NC6=O)ccn5)CC4)c3)c12] | MEK |
| 419 | 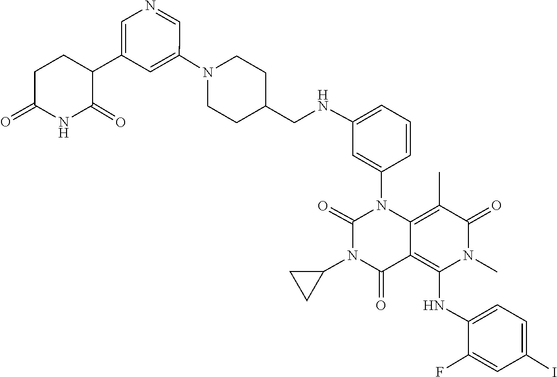 [SMILES: Cc1c(=O)n(C)c(Nc2ccc(I)cc2F)c2c(=O)n(C3CC3)c(=O)n(-c3cccc(NCC4CCN(c5cncc(C6CCC(=O)NC6=O)c5)CC4)c3)c12] | MEK |
| 420 | 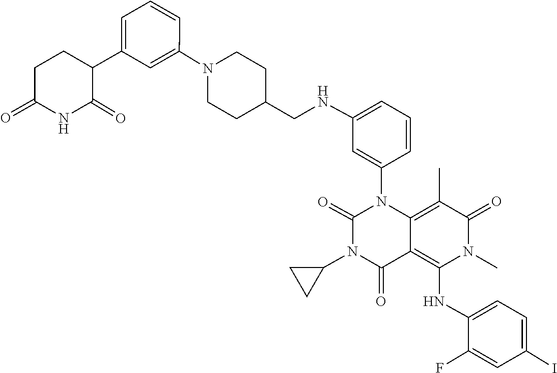 [SMILES: Cc1c(=O)n(C)c(Nc2ccc(I)cc2F)c2c(=O)n(C3CC3)c(=O)n(-c3cccc(NCC4CCN(c5cccc(C6CCC(=O)NC6=O)c5)CC4)c3)c12] | MEK |
| 421 | 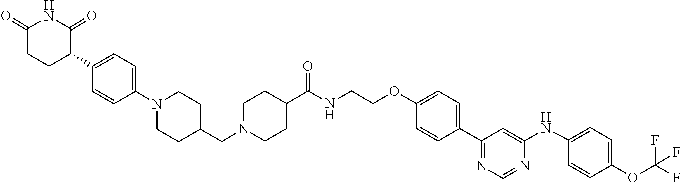 [SMILES: O=C1CC[C@@H](c2ccc(N3CCC(CN4CCC(C(=O)NCCOc5ccc(-c6cc(Nc7ccc(OC(F)(F)F)cc7)ncn6)cc5)CC4)CC3)cc2)C(=O)N1] | BCR- ABL |
| 422 | 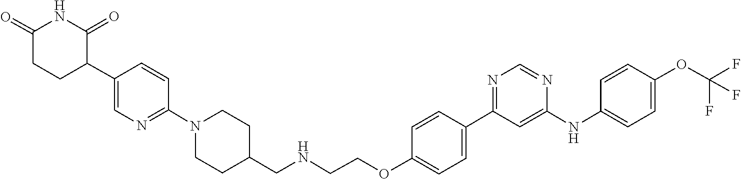 [SMILES: O=C1CCC(c2ccc(N3CCC(CNCCOc4ccc(-c5cc(Nc6ccc(OC(F)(F)F)cc6)ncn5)cc4)CC3)nc2)C(=O)N1] | BCR- ABL |
| 423 | 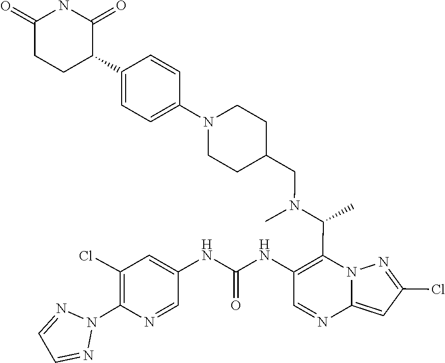 | MALT1 |
| 424 | 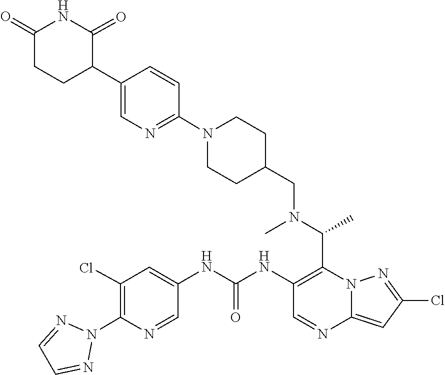 [SMILES: C[C@H](c1c(NC(=O)Nc2cnc(-n3nccn3)c(Cl)c2)cnc2cc(Cl)nn12)N(C)CC1CCN(c2ccc(C3CCC(=O)NC3=O)cn2)CC1] | MALT1 |
| 425 | 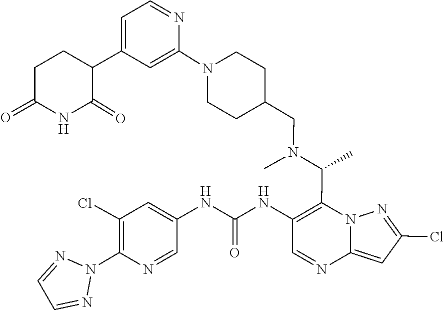 [SMILES: C[C@H](c1c(NC(=O)Nc2cnc(-n3nccn3)c(Cl)c2)cnc2cc(Cl)nn12)N(C)CC1CCN(c2cc(C3CCC(=O)NC3=O)ccn2)CC1] | MALT1 |
| 426 | 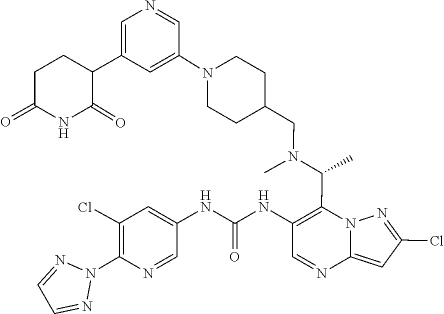 [SMILES: C[C@H](c1c(NC(=O)Nc2cnc(-n3nccn3)c(Cl)c2)cnc2cc(Cl)nn12)N(C)CC1CCN(c2cncc(C3CCC(=O)NC3=O)c2)CC1] | MALT1 |
| 427 | 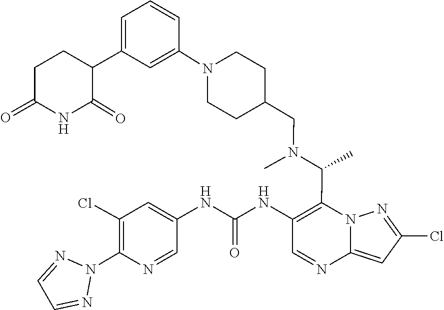 [SMILES: C[C@H](c1c(NC(=O)Nc2cnc(-n3nccn3)c(Cl)c2)cnc2cc(Cl)nn12)N(C)CC1CCN(c2cccc(C3CCC(=O)NC3=O)c2)CC1] | MALT1 |
| 428 | 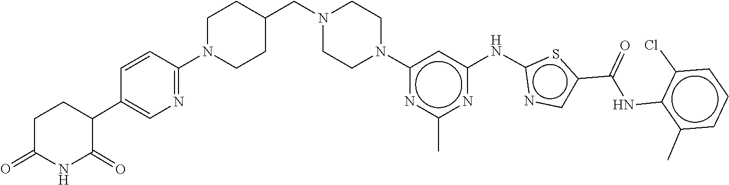 | BCR- ABL |
| 429 | 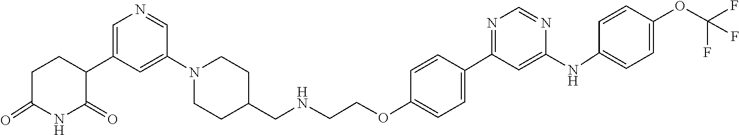 [SMILES: O=C1CCC(c2cncc(N3CCC(CNCCOc4ccc(-c5cc(Nc6ccc(OC(F)(F)F)cc6)ncn5)cc4)CC3)c2)C(=O)N1] | BCR- ABL |
| 430 | 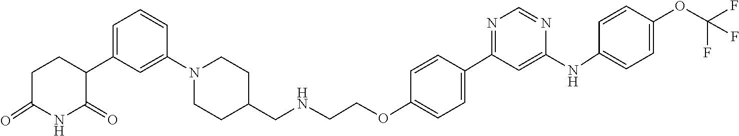 [SMILES: O=C1CCC(c2cccc(N3CCC(CNCCOc4ccc(-c5cc(Nc6ccc(OC(F)(F)F)cc6)ncn5)cc4)CC3)c2)C(=O)N1] | BCR- ABL |
| 431 | 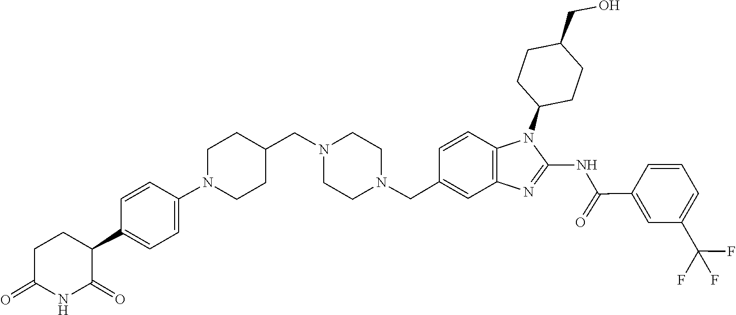 [SMILES: O=C1CC[C@@H](c2ccc(N3CCC(CN4CCN(Cc5ccc6c(c5)nc(NC(=O)c5cccc(C(F)(F)F)c5)n6[C@H]5CC[C@@H](CO)CC5)CC4)CC3)cc2)C(=O)N1] | IRAK1 |
| 432 | 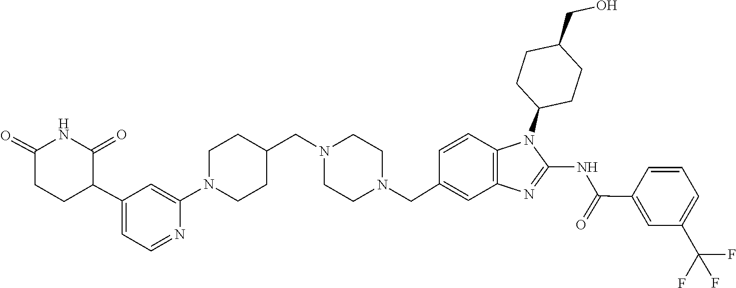 [SMILES: O=C1CCC(c2ccnc(N3CCC(CN4CCN(Cc5ccc6c(c5)nc(NC(=O)c5cccc(C(F)(F)F)c5)n6[C@H]5CC[C@@H](CO)CC5)CC4)CC3)c2)C(=O)N1] | IRAK1 |
| 433 | 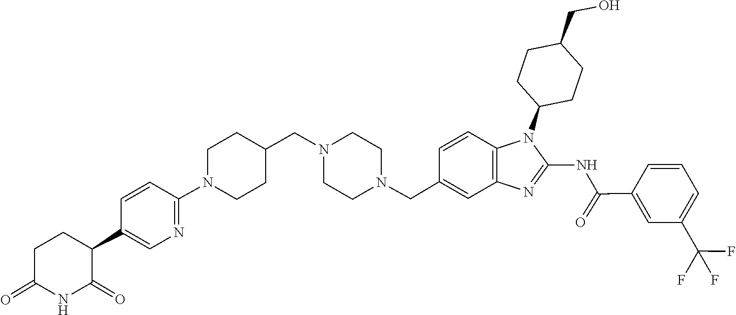 [SMILES: O=C1CC[C@@H](c2ccc(N3CCC(CN4CCN(Cc5ccc6c(c5)nc(NC(=O)c5cccc(C(F)(F)F)c5)n6[C@H]5CC[C@@H](CO)CC5)CC4)CC3)nc2)C(=O)N1] | IRAK1 |
| 434 | 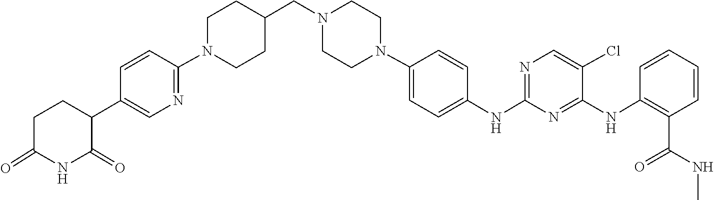 [SMILES: CNC(=O)c1ccccc1Nc1nc(Nc2ccc(N3CCN(CC4CCN(c5ccc(C6CCC(=O)NC6=O)cn5)CC4)CC3)cc2)ncc1Cl] | Pan kinase |
| 435 | 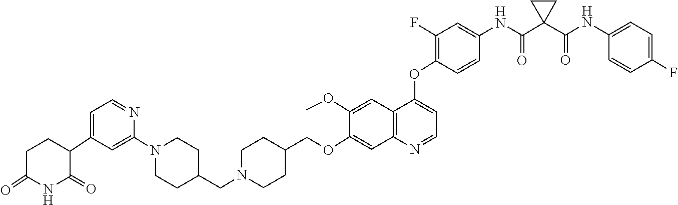 [SMILES: COc1cc2c(Oc3ccc(NC(=O)C4(C(=O)Nc5ccc(F)cc5)CC4)cc3F)ccnc2cc1OCC1CCN(CC2CCN(c3cc(C4CCC(=O)NC4=O)ccn3)CC2)CC1] | Pan kinase |
| 436 | 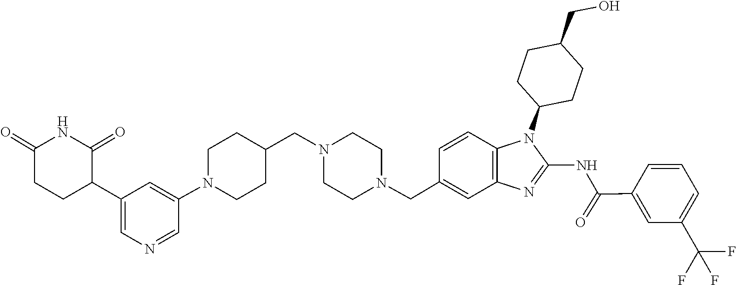 [SMILES: O=C1CCC(c2cncc(N3CCC(CN4CCN(Cc5ccc6c(c5)nc(NC(=O)c5cccc(C(F)(F)F)c5)n6[C@H]5CC[C@@H](CO)CC5)CC4)CC3)c2)C(=O)N1] | IRAK1 |
| 437 | 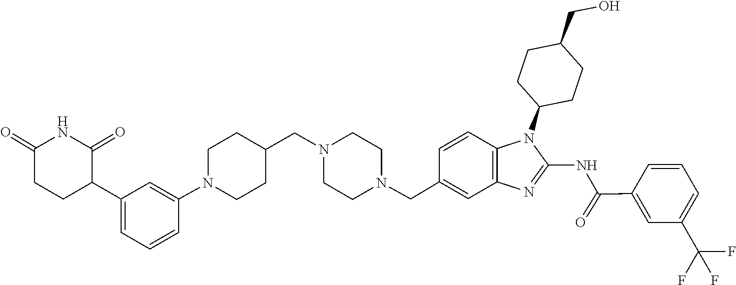 [SMILES: O=C1CCC(c2cccc(N3CCC(CN4CCN(Cc5ccc6c(c5)nc(NC(=O)c5cccc(C(F)(F)F)c5)n6[C@H]5CC[C@@H](CO)CC5)CC4)CC3)c2)C(=O)N1] | IRAK1 |
| 438 | 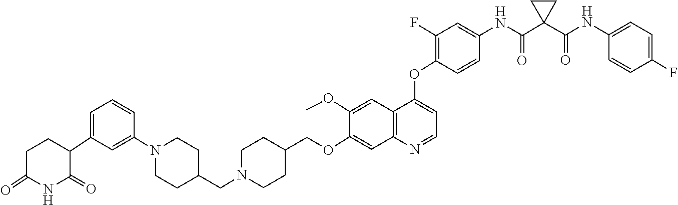 [SMILES: COc1cc2c(Oc3ccc(NC(=O)C4(C(=O)Nc5ccc(F)cc5)CC4)cc3F)ccnc2cc1OCC1CCN(CC2CCN(c3cccc(C4CCC(=O)NC4=O)c3)CC2)CC1] | Pan kinase |
| 439 | 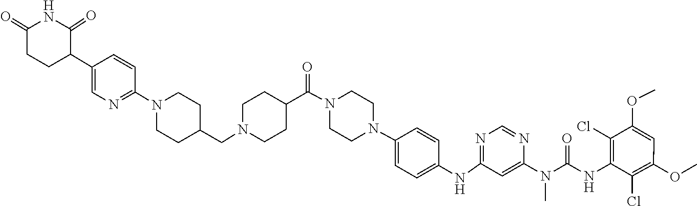 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cn7)CC6)CC5)CC4)cc3)ncn2)c1Cl] | SHP-2 |
| 440 | 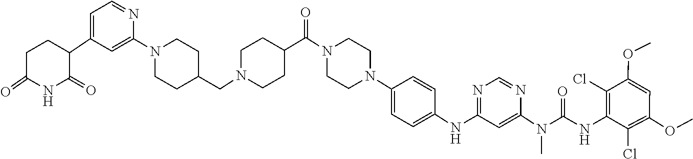 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] | FGFR1/3 |
| 441 | 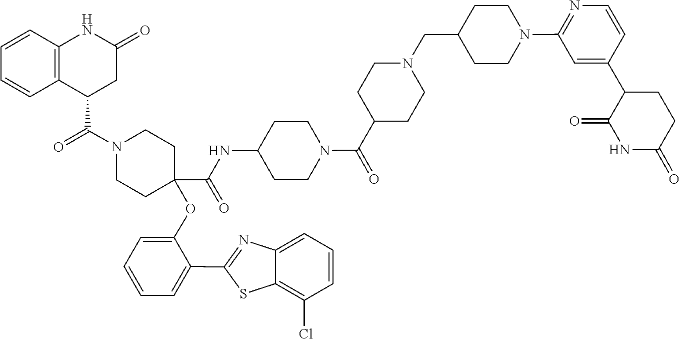 [SMILES: O=C1CCC(c2ccnc(N3CCC(CN4CCC(C(=O)N5CCC(NC(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] | COVID |
| 442 | 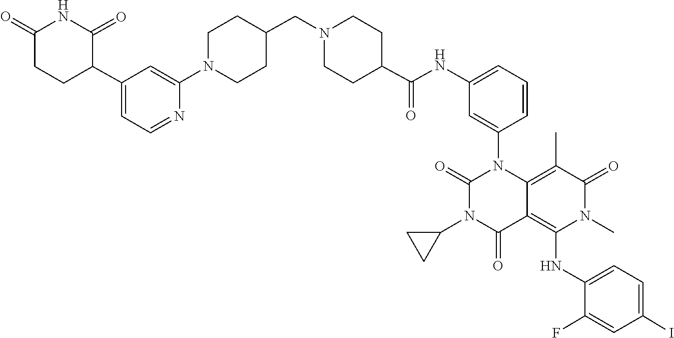 [SMILES: Cc1c(=O)n(C)c(Nc2ccc(I)cc2F)c2c(=O)n(C3CC3)c(=O)n(-c3cccc(NC(=O)C4CCN(CC5CCN(c6cc(C7CCC(=O)NC7=O)ccn6)CC5)CC4)c3)c12] | MEK |
| 443 | 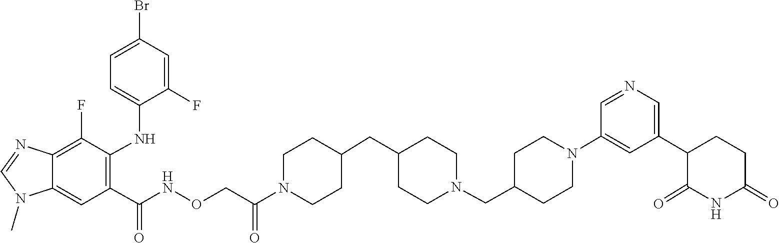 [SMILES: Cn1cnc2c(F)c(Nc3ccc(Br)cc3F)c(C(=O)NOCC(=O)N3CCC(CC4CCN(CC5CCN(c6cncc(C7CCC(=O)NC7=O)c6)CC5)CC4)CC3)cc21] | MEK |
| 444 | 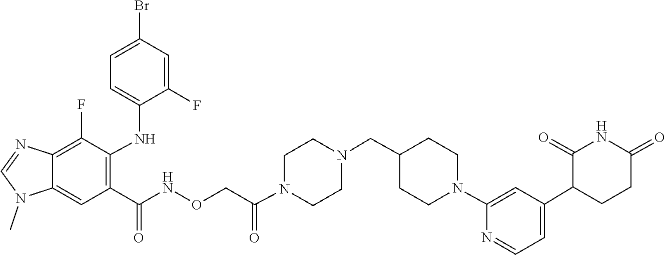 [SMILES: Cn1cnc2c(F)c(Nc3ccc(Br)cc3F)c(C(=O)NOCC(=O)N3CCN(CC4CCN(c5cc(C6CCC(=O)NC6=O)ccn5)CC4)CC3)cc21] | MEK |
| 445 | 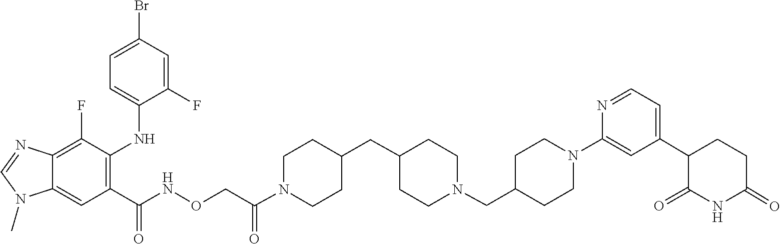 [SMILES: Cn1cnc2c(F)c(Nc3ccc(Br)cc3F)c(C(=O)NOCC(=O)N3CCC(CC4CCN(CC5CCN(c6cc(C7CCC(=O)NC7=O)ccn6)CC5)CC4)CC3)cc21] | MEK |
| 446 | 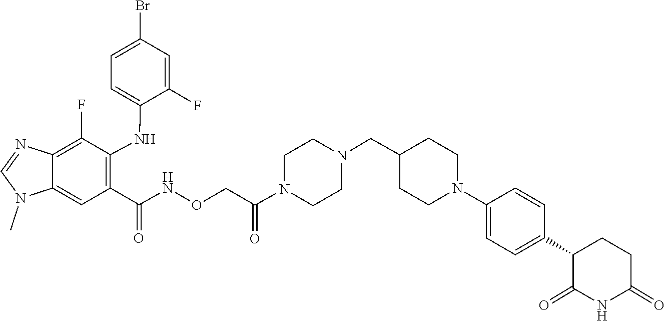 [SMILES: Cn1cnc2c(F)c(Nc3ccc(Br)cc3F)c(C(=O)NOCC(=O)N3CCN(CC4CCN(c5ccc([C@@H]6CCC(=O)NC6=O)cc5)CC4)CC3)cc21] | MEK |
| 447 | 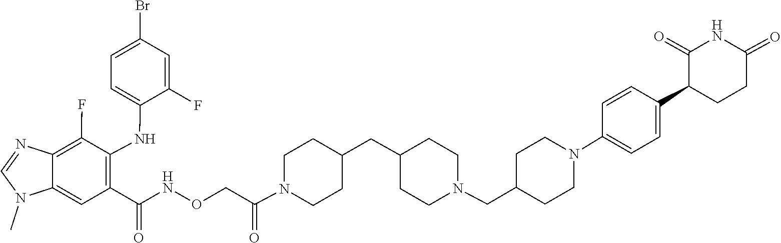 [SMILES: Cn1cnc2c(F)c(Nc3ccc(Br)cc3F)c(C(=O)NOCC(=O)N3CCC(CC4CCN(CC5CCN(c6ccc([C@@H]7CCC(=O)NC7=O)cc6)CC5)CC4)CC3)cc21] | MEK |
| 448 | 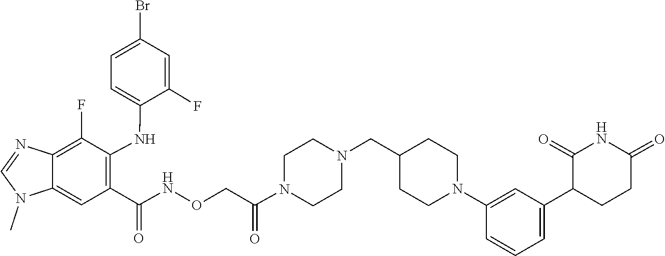 [SMILES: Cn1cnc2c(F)c(Nc3ccc(Br)cc3F)c(C(=O)NOCC(=O)N3CCN(CC4CCN(c5cccc(C6CCC(=O)NC6=O)c5)CC4)CC3)cc21] | MEK |
| 449 | 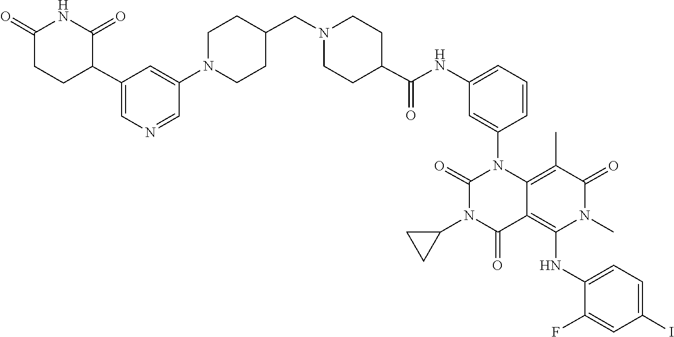 [SMILES: Cc1c(=O)n(C)c(Nc2ccc(I)cc2F)c2c(=O)n(C3CC3)c(=O)n(-c3cccc(NC(=O)C4CCN(CC5CCN(c6cncc(C7CCC(=O)NC7=O)c6)CC5)CC4)c3)c12] | MEK |
| 450 | 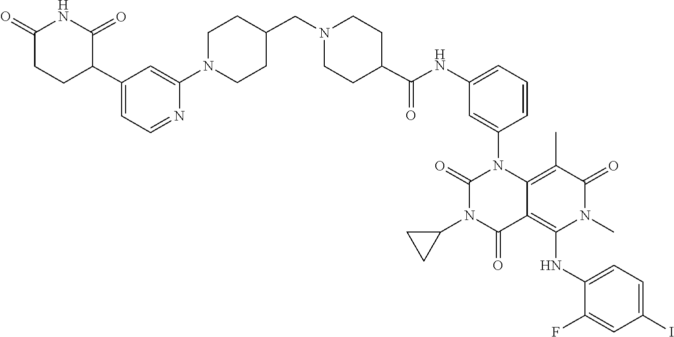 [SMILES: Cc1c(=O)n(C)c(Nc2ccc(I)cc2F)c2c(=O)n(C3CC3)c(=O)n(-c3cccc(NC(=O)C4CCN(CC5CCN(c6cc(C7CCC(=O)NC7=O)ccn6)CC5)CC4)c3)c12] | MEK |
| 451 | 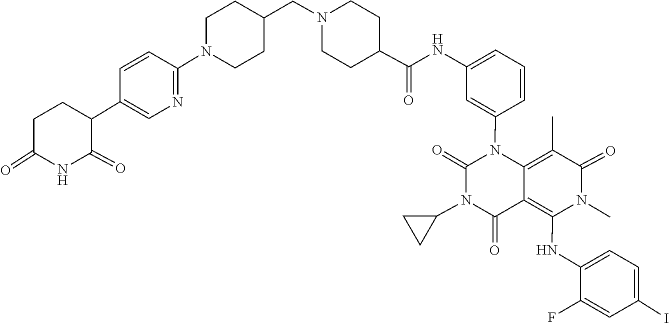 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] | MEK |
| 452 | 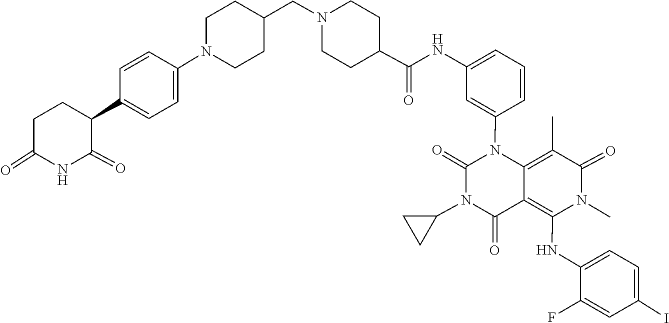 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc([C@@H]7CCC(=O)NC7=O)cc6)CC5)CC4)c3)c12] | MEK |
| 453 | 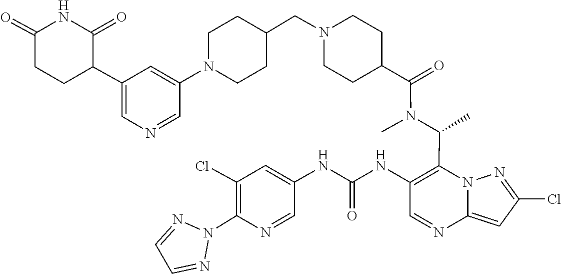 [SMILES: C[C@H](c1c(NC(=O)Nc2cnc(-n3nccn3)c(Cl)c2)cnc2cc(Cl)nn12)N(C)C(=O)C1CCN(CC2CCN(c3cncc(C4CCC(=O)NC4=O)c3)CC2)CC1] | MALT1 |
| 454 | 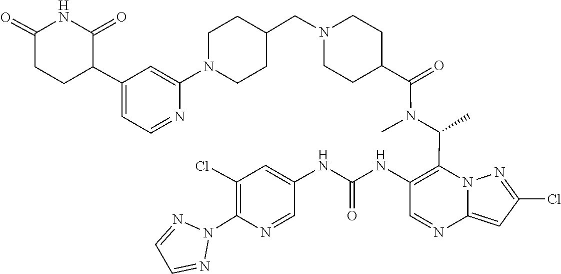 [SMILES: C[C@H](c1c(NC(=O)Nc2cnc(-n3nccn3)c(Cl)c2)cnc2cc(Cl)nn12)N(C)C(=O)C1CCN(CC2CCN(c3cc(C4CCC(=O)NC4=O)ccn3)CC2)CC1] | MALT1 |
| 455 | 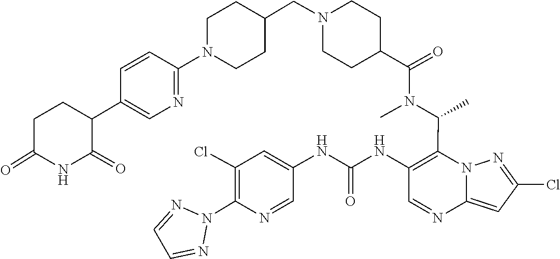 [SMILES: C[C@H](c1c(NC(=O)Nc2cnc(-n3nccn3)c(Cl)c2)cnc2cc(Cl)nn12)N(C)C(=O)C1CCN(CC2CCN(c3ccc(C4CCC(=O)NC4=O)cn3)CC2)CC1] | MALT1 |
| 456 | 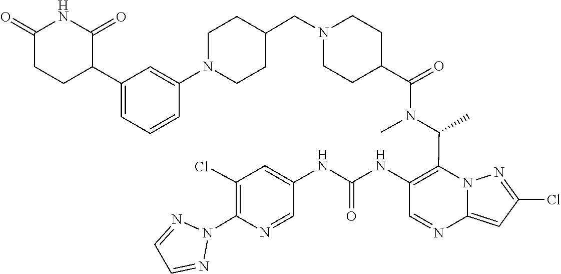 [SMILES: C[C@H](c1c(NC(=O)Nc2cnc(-n3nccn3)c(Cl)c2)cnc2cc(Cl)nn12)N(C)C(=O)C1CCN(CC2CCN(c3cccc(C4CCC(=O)NC4=O)c3)CC2)CC1] | MALT1 |
| 457 | 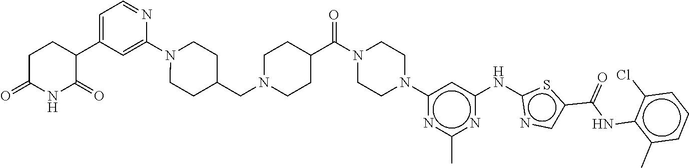 | BCR- ABL |
| 458 | 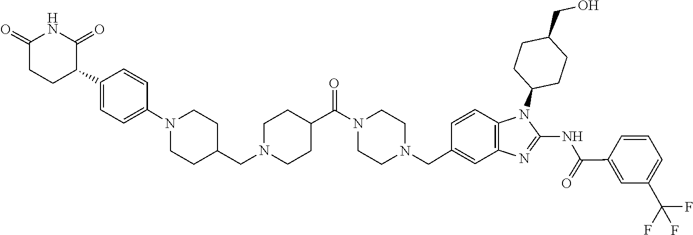 [SMILES: O=C1CC[C@@H](c2ccc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)cc2)C(=O)N1] | IRAK1 |
| 459 | 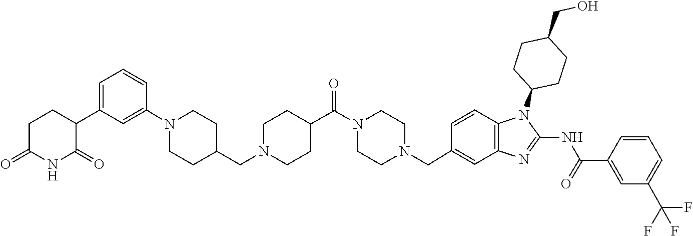 [SMILES: O=C1CCC(c2cccc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)c2)C(=O)N1] | IRAK1 |
| 460 | 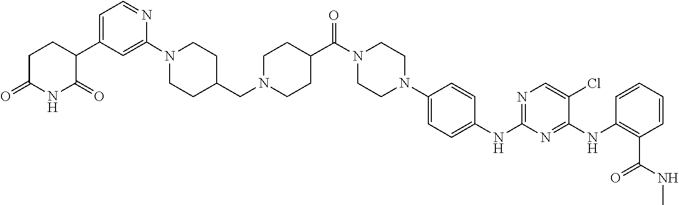 [SMILES: CNC(=O)c1ccccc1Nc1nc(Nc2ccc(N3CCN(C(=O)C4CCN(CC5CCN(c6cc(C7CCC(=O)NC7=O)ccn6)CC5)CC4)CC3)cc2)ncc1Cl] | Pan kinase |
| 461 | 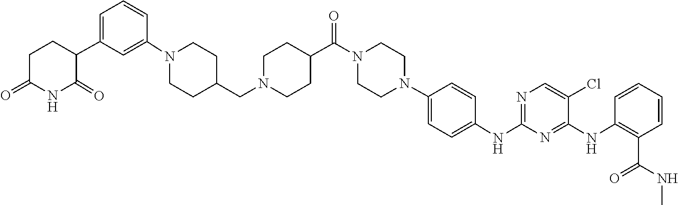 [SMILES: CNC(=O)c1ccccc1Nc1nc(Nc2ccc(N3CCN(C(=O)C4CCN(CC5CCN(c6cccc(C7CCC(=O)NC7=O)c6)CC5)CC4)CC3)cc2)ncc1Cl] | Pan kinase |
| 462 | 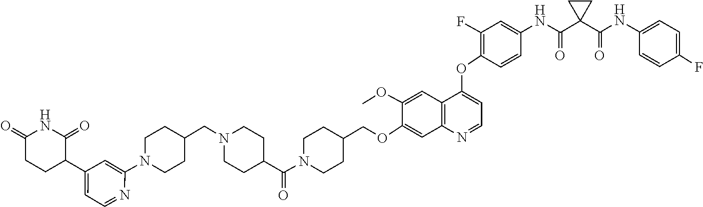 [SMILES: COc1cc2c(Oc3ccc(NC(=O)C4(C(=O)Nc5ccc(F)cc5)CC4)cc3F)ccnc2cc1OCC1CCN(C(=O)C2CCN(CC3CCN(c4cc(C5CCC(=O)NC5=O)ccn4)CC3)CC2)CC1] | Pan kinase |
| 463 | 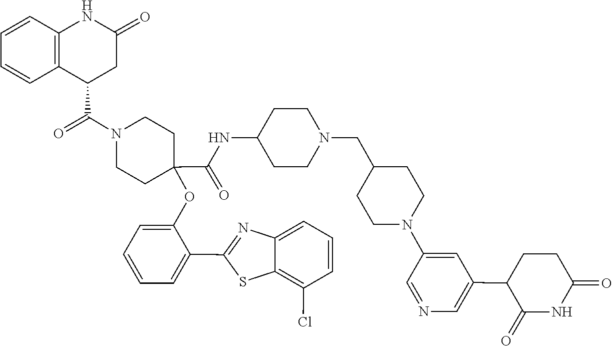 [SMILES: O=C1CCC(c2cncc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)c2)C(=O)N1] | COVID |
| 464 | 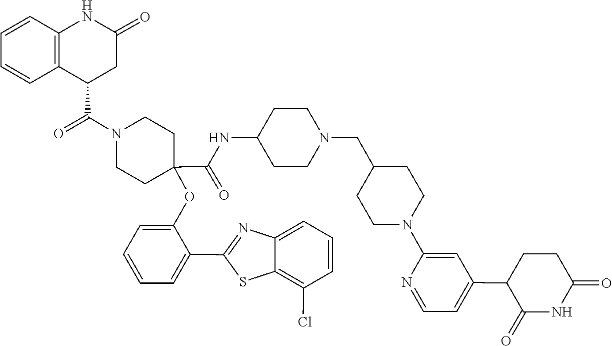 [SMILES: O=C1CCC(c2ccnc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)c2)C(=O)N1] | COVID |
| 465 | 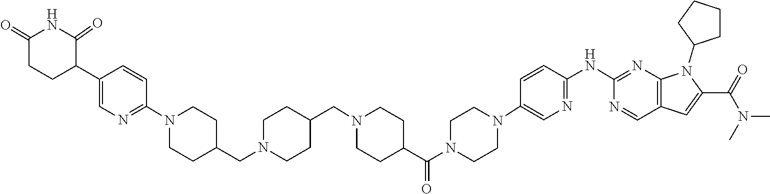 [SMILES: CN(C)C(=O)c1cc2cnc(Nc3ccc(N4CCN(C(=O)C5CCN(CC6CCN(CC7CCN(c8ccc(C9CCC(=O)NC9=O)cn8)CC7)CC6)CC5)CC4)cn3)nc2n1C1CCCC1] | CDK4/6 |
| 466 | 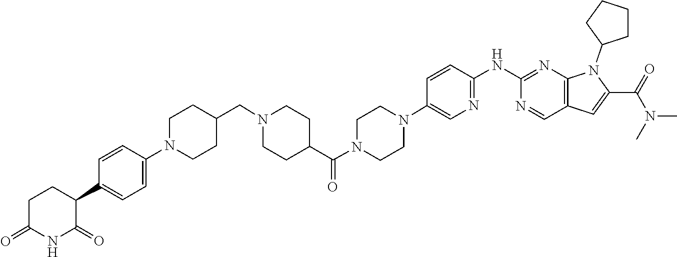 [SMILES: CN(C)C(=O)c1cc2cnc(Nc3ccc(N4CCN(C(=O)C5CCN(CC6CCN(c7ccc([C@@H]8CCC(=O)NC8=O)cc7)CC6)CC5)CC4)cn3)nc2n1C1CCCC1] | CDK4/6 |
| 467 | 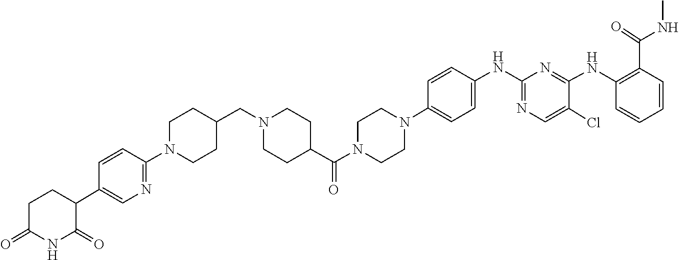 [SMILES: CNC(=O)c1ccccc1Nc1nc(Nc2ccc(N3CCN(C(=O)C4CCN(CC5CCN(c6ccc(C7CCC(=O)NC7=O)cn6)CC5)CC4)CC3)cc2)ncc1Cl] | SHP-2 |
| 468 | 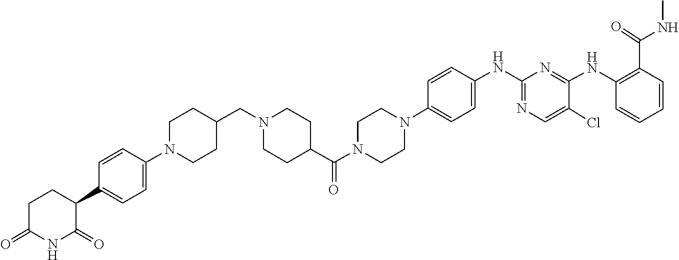 [SMILES: CNC(=O)c1ccccc1Nc1nc(Nc2ccc(N3CCN(C(=O)C4CCN(CC5CCN(c6ccc([C@@H]7CCC(=O)NC7=O)cc6)CC5)CC4)CC3)cc2)ncc1Cl] | Pan kinase |
| 469 | 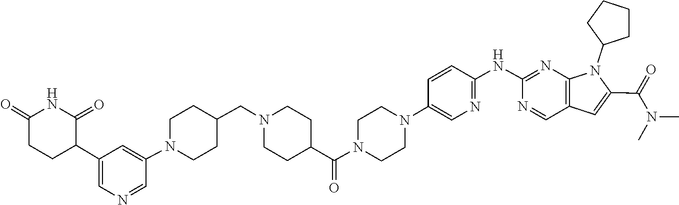 [SMILES: CN(C)C(=O)c1cc2cnc(Nc3ccc(N4CCN(C(=O)C5CCN(CC6CCN(c7cncc(C8CCC(=O)NC8=O)c7)CC6)CC5)CC4)cn3)nc2n1C1CCCC1] | CDK4/6 |
| 470 | 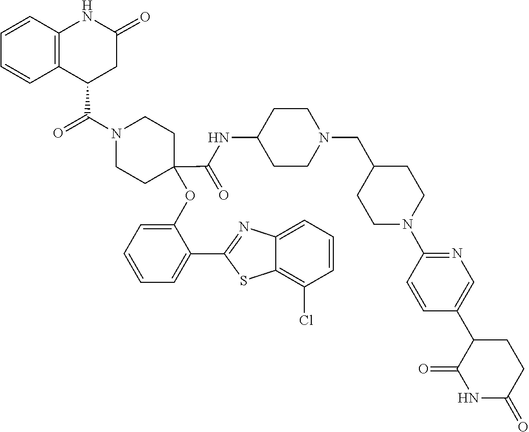 [SMILES: O=C1CCC(c2ccc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)nc2)C(=O)N1] | COVID |
| 471 | 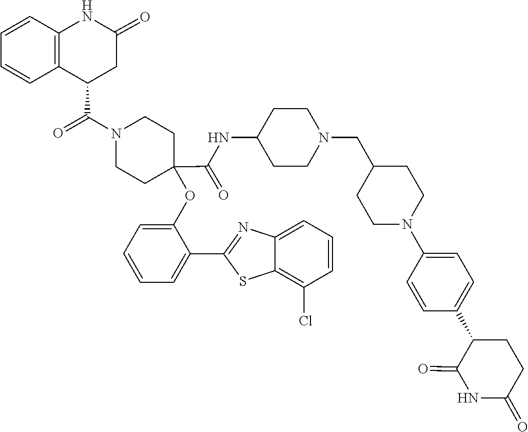 [SMILES: O=C1CC[C@@H](c2ccc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)cc2)C(=O)N1] | COVID |
| 472 | 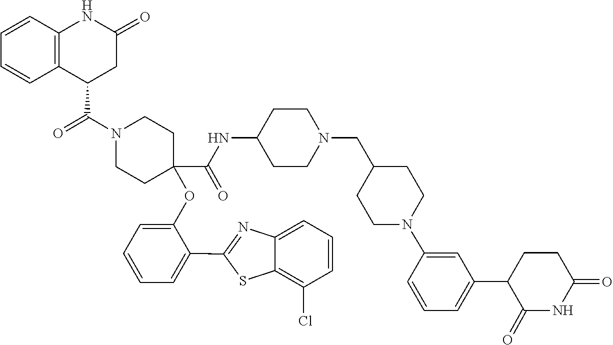 [SMILES: O=C1CCC(c2cccc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)c2)C(=O)N1] | COVID |
| 473 | 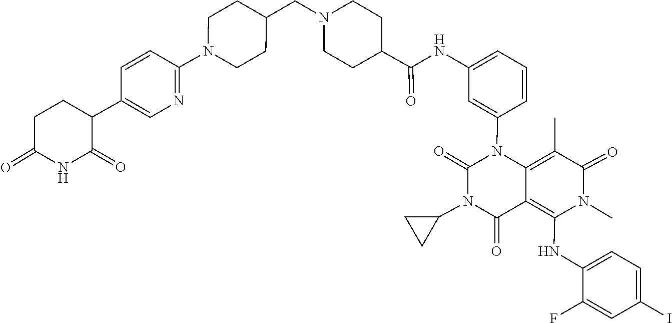 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] | MEK |
| 474 | 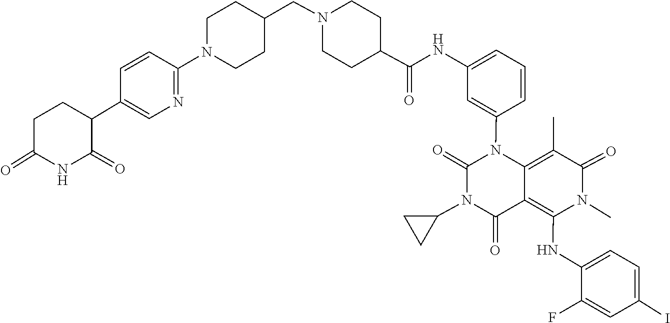 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] | MEK |
| 475 | 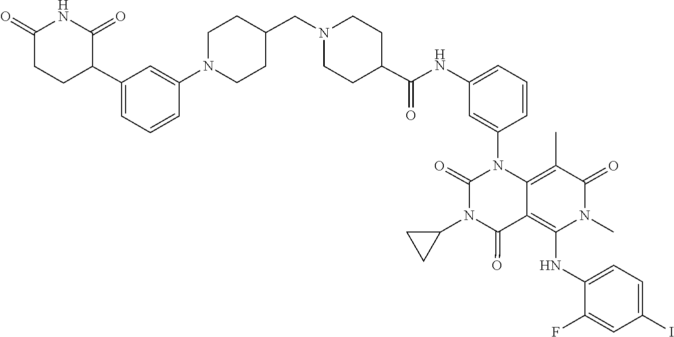 [SMILES: Cc1c(=O)n(C)c(Nc2ccc(I)cc2F)c2c(=O)n(C3CC3)c(=O)n(-c3cccc(NC(=O)C4CCN(CC5CCN(c6cccc(C7CCC(=O)NC7=O)c6)CC5)CC4)c3)c12] | MEK |
| 476 | 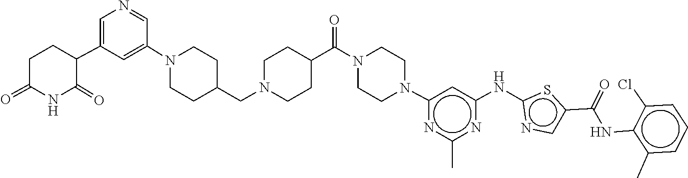 | BCR- ABL |
| 477 | 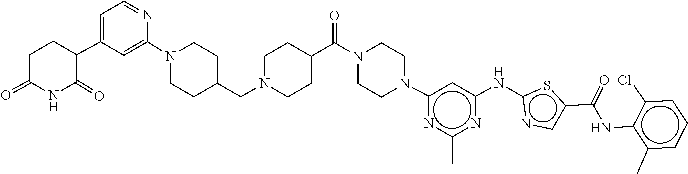 | BCR- ABL |
| 478 | 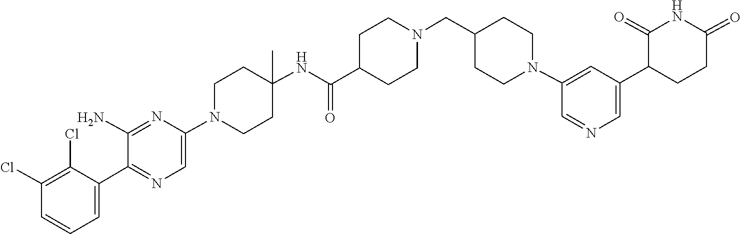 [SMILES: CC1(NC(=O)C2CCN(CC3CCN(c4cncc(C5CCC(=O)NC5=O)c4)CC3)CC2)CCN(c2cnc(-c3cccc(Cl)c3Cl)c(N)n2)CC1] | SHP-2 |
| 479 | 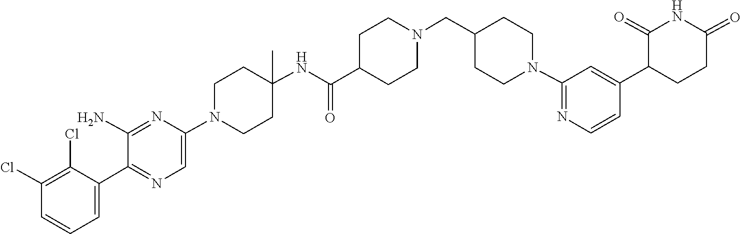 [SMILES: CC1(NC(=O)C2CCN(CC3CCN(c4cc(C5CCC(=O)NC5=O)ccn4)CC3)CC2)CCN(c2cnc(-c3cccc(Cl)c3Cl)c(N)n2)CC1] | SHP-2 |
| 480 | 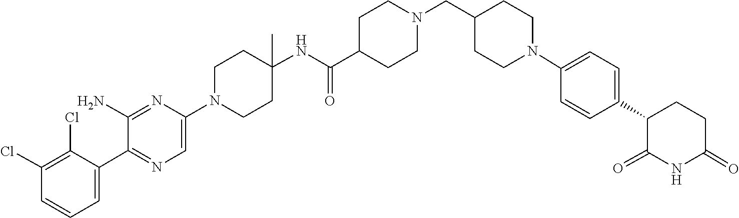 [SMILES: CC1(NC(=O)C2CCN(CC3CCN(c4ccc([C@@H]5CCC(=O)NC5=O)cc4)CC3)CC2)CCN(c2cnc(-c3cccc(Cl)c3Cl)c(N)n2)CC1] | SHP-2 |
| 481 | 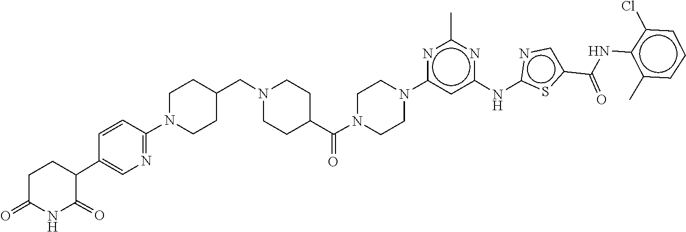 | BCR- ABL |
| 482 | 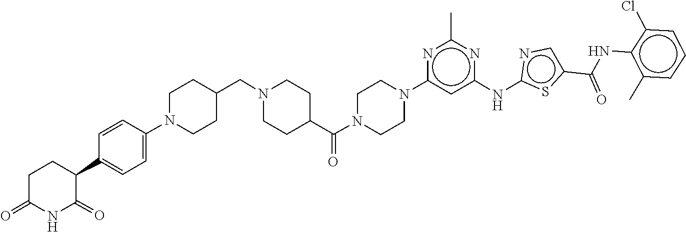 | BCR- ABL |
| 483 | 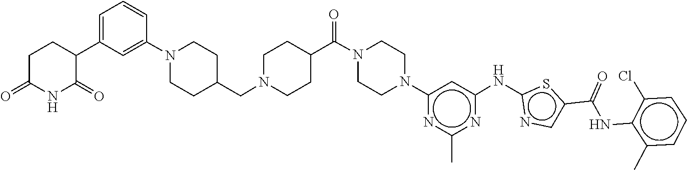 | BCR- ABL |
| 484 | 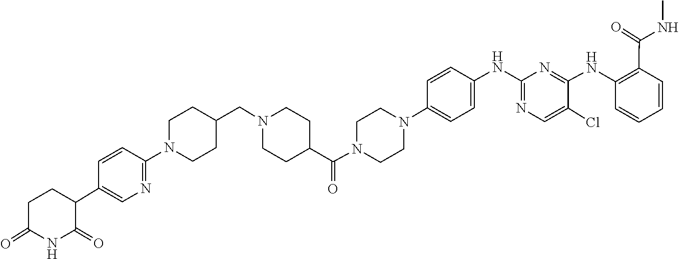 [SMILES: CNC(=O)c1ccccc1Nc1nc(Nc2ccc(N3CCN(C(=O)C4CCN(CC5CCN(c6ccc(C7CCC(=O)NC7=O)cn6)CC5)CC4)CC3)cc2)ncc1Cl] | SHP-2 |
| 485 | 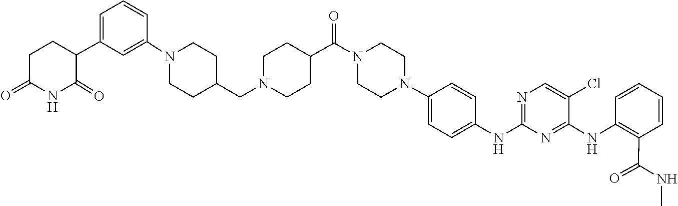 [SMILES: CNC(=O)c1ccccc1Nc1nc(Nc2ccc(N3CCN(C(=O)C4CCN(CC5CCN(c6cccc(C7CCC(=O)NC7=O)c6)CC5)CC4)CC3)cc2)ncc1Cl] | SHP-2 |

In certain embodiments, the compound is selected from the compounds in Table 1 and pharmaceutically acceptable salts thereof.

Compounds provided herein can be prepared or synthesized according to any technique deemed suitable by the person of skill in the art. Exemplary synthetic schemes are described below.

### Uses of the Compounds and Compositions

The heterobifunctional compounds described herein are useful for degrading one or more target proteins. In certain embodiments, the target protein is selected from the group consisting of BTK; ITK/TSK; BRD4; FLT-3; BAF; BAF complex; MCL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases in biological samples or in patients via an ubiquitin proteolytic pathway. Thus, an embodiment of this disclosure provides a method of treating a disease or disorder, wherein said disease or disorder is selected from the group consisting of a BTK; ITK/TSK; BRD4; FLT-3; BAF; BAF complex; McL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and/or kinase-mediated disease or disorder. As used herein, the term “BTK; ITK/TSK; BRD4; FLT-3; BAF; BAF complex; MCL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and/or kinase-mediated disease or disorder” means any disease, disorder, or other deleterious condition modulated by BTK; ITK/TSK; BRD4; FLT-3; BAF; BAF complex; McL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and/or kinases. In some instances, a BTK; ITK/TSK; BRD4; FLT-3; BAF; BAF complex; McL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and/or kinase-mediated disease or disorder is a proliferative disorder, an autoimmune disorder, or an inflammatory disorder. Examples of proliferative disorders include cancer.

In certain embodiments, the cancer is any cancer deemed suitable to the practitioner of skill. In particular embodiments, the cancer comprises a solid tumor. In certain embodiments, the cancer is a B cell malignancy. In certain embodiments, the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), and central nervous system (CNS) lymphoma. In certain embodiments, the cancer is chronic lymphocytic leukemia. In certain embodiments, the cancer is small cell lymphoma. In certain embodiments, the cancer is follicular lymphoma. In certain embodiments, the cancer is diffuse large B-cell lymphoma. In certain embodiments, the cancer is non-Hodgkin lymphoma. In certain embodiments, the cancer is mantle cell lymphoma. In certain embodiments, the cancer is marginal zone lymphoma. In certain embodiments, the cancer is Waldenstrom macroglobulinemia. In certain embodiments, the cancer is small lymphocytic lymphoma (SLL). In certain embodiments, the cancer is CNS lymphoma. In certain embodiments, the cancer is transformed CLL or Richter's transformation.

In another aspect, provided herein are methods of degrading a target protein in a subject in need thereof. The methods comprise the step of administering to the subject an amount of a heterobifunctional compound capable of inducing proteolytic degradation of the target protein. In certain embodiments, the amount is effective to degrade a target protein in the subject. The target protein can be expressed in any cells or tissues of the subject. In certain embodiments, the target protein is expressed in splenocytes. In certain embodiments, the target protein is expressed in peripheral blood mononuclear cells.

In the methods, the heterobifunctional compounds comprise a moiety capable of specifically binding to a target protein. In certain embodiments, the target protein is selected from the group consisting of BTK; ITK/TSK; BRD4; FLT-3; BAF; BAF complex; McL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases and further comprise a moiety capable of recruiting an ubiquitin ligase to degrade a target protein, wherein said target protein is selected from the group consisting of BTK; ITK/TSK; BRD4; FLT-3; BAF; BAF complex; MCL-1; STAT3; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases. Particular compounds are described herein. The compounds can be administered in any form, including pharmaceutically acceptable salts and pharmaceutical compositions.

The heterobifunctional compound can be administered at any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg. In certain embodiments, the dose is selected from the group consisting of 100 mg/kg, 200 mg/kg, 300 mg/kg, 450 mg/kg, 600 mg/kg, 800 mg/kg, and 1000 mg/kg. In certain embodiments, the dose is about 25 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The dose can be administered on a schedule deemed suitable by the person of skill in the art. In certain embodiments, the dose is administered once per day. In certain embodiments, the dose is administered twice per day. In certain embodiments, the dose is administered three times per day. In certain embodiments, the dose is administered four times per day. In certain embodiments, the dose is administered in divided doses. In certain embodiments, the dose is administered in two divided doses per day. In certain embodiments, the dose is administered in three divided doses per day. In certain embodiments, the dose is administered in four divided doses per day.

Dosing can continue for any length of time deemed suitable by the person of skill in the art. In certain embodiments, the dose is administered daily for fourteen days. In certain embodiments, the dose is administered daily for thirteen days. In certain embodiments, the dose is administered daily for twelve days. In certain embodiments, the dose is administered daily for eleven days. In certain embodiments, the dose is administered daily for ten days. In certain embodiments, the dose is administered daily for nine days. In certain embodiments, the dose is administered daily for eight days. In certain embodiments, the dose is administered daily for seven days. In certain embodiments, the dose is administered daily for six days. In certain embodiments, the dose is administered daily for five days. In certain embodiments, the dose is administered daily for four days. In certain embodiments, the dose is administered daily for three days. In certain embodiments, the dose is administered daily for two days. In certain embodiments, the dose is administered for one day.

In the dosing schedule, the doses can be administered on consecutive days or cyclically, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the dose is administered weekly. In certain embodiments, the dose is administered twice per week. In certain embodiments, the dose is administered three times per week.

In certain embodiments, the dose(s) are administered for a period of time with a first interval between dose(s), and then the dose(s) are re-administered for a period of time following the first interval between dose(s), wherein this dosing regimen can be repeated (i.e., cyclically or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered by any route of administration deemed suitable by the practitioner of skill. In certain embodiments, the dose is administered orally. Formulations and techniques for administration are described in detail below.

In certain embodiments, term “cancer” includes, but is not limited to, the following cancers: Epidermoid Oral: buccal cavity, lip, tongue, mouth, pharynx, squamous cell carcinoma of the head and neck (HNSCC); Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); Gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma (MM), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenstrom's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis; Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metatstaic melanoma.

In certain embodiments, examples of autoimmune disorders include uticaria, graft-versus-host disease (GVHD), acute graft-versus-host disease, pemphigus vulgaris, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, axonal and neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive Arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopenic purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)).

In certain embodiments, term “inflammatory disease” includes, but is not limited to, the following inflammatory diseases: encephalitis, myelitis, meningitis, arachnoiditis, neuritis, dacryoadenitis, scleritis, episcleritis, keratitis, retinitis, chorioretinitis, blepharitis, conjunctivitis, uveitis, otitisexterna, otitismedia, labyrinthitis, mastoiditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascendingcholangitis, cholecystitis, pancreatitis, peritonitis, dermatitis, folliculitis, cellulitis, hidradenitis, arthritis, dermatomyositis, softtissue, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminalvesiculitis, balanitis, posthitis, balanoposthitis, chorioamnionitis, funisitis, omphalitis, insulitis, hypophysitis, thyroiditis, parathyroiditis, adrenalitis, lymphangitis, and lymphadenitis.

### Formulations and Administration

Pharmaceutical Compositions

The compounds described herein can be formulated into pharmaceutical compositions that further comprise a pharmaceutically acceptable carrier, diluent, excipient, or vehicle. In one embodiment, this disclosure provides a pharmaceutical composition comprising a compound described above, and a pharmaceutically acceptable carrier, diluent, excipient, or vehicle. In one embodiment, provided herein are pharmaceutical compositions comprising effective amounts of compound of this disclosure or a pharmaceutically acceptable salts thereof and one or more pharmaceutically acceptable carriers, diluents, excipients, or vehicles.

According to another embodiment, the description provides a composition comprising a compound herein or a pharmaceutically acceptable salt thereof and a pharmaceutically acceptable carrier, or vehicle. Pharmaceutical compositions of this description comprise a therapeutically effective amount of a compound of Formula (I), wherein a “therapeutically effective amount” is an amount that is effective to measurably degrade a target protein. In certain embodiments, the target protein is selected from the group consisting of BTK; ITK/TSK; BRD4; FLT-3; BAF; BAF complex; MCL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases. In certain embodiments, to reduce the amount of to a target protein, wherein said target protein is selected from the group consisting of BTK; ITK/TSK; BRD4; FLT-3; BAF; BAF complex; MCL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases. In certain embodiments, in a biological sample or in a patient. In certain embodiments effective in treating and/or ameliorating a disease or disorder that is mediated by a target protein. In certain embodiments the target protein is selected from the group consisting of BTK; ITK/TSK; BRD4; FLT-3; BAF; BAF complex; MCL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases.

It also will be appreciated that certain compounds of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁-4 alkyl)₄ salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade,” means a measurable reduction in (a) a target protein activity, between a sample comprising a compound of this description and a target protein, and an equivalent sample comprising a target protein, in the absence of said compound; or (b) the concentration of the target protein in a sample over time. In certain embodiments, said target protein is selected from the group consisting of BTK; ITK/TSK; BRD4; FLT-3; BAF; BAF complex; MCL-1; STAT3; BCR-ABL; CDK4; CDK6; SHP-2; FGFR1; FGFR3; FGFR1 fusions; FGFR3 fusions; MDM2; TRIM24; SARS-COV2; PI3K; PI3K delta; MEK; BCR-ABL; MLL; MALT1; IRAK1; and kinases. In certain embodiments, said target protein is selected from the group consisting of CDK4, CDK6, SHP-2, FGFR1, FGFR3, FGFR1 fusions, FGFR3 fusions, MDM2, TRIM24, SARS-COV2 protein, PI3K, PI3K delta, MEK, BCR-ABL, MLL, MALT1; IRAK1; and kinases.

### Administration

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a certain part of the intestinal tract, optionally, in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

Depending upon the particular condition, or disease, to be treated or prevented, additional therapeutic agents, which are normally administered to treat or prevent that condition, also may be present in the compositions of this disclosure. As used herein, additional therapeutic agents that are normally administered to treat or prevent a particular disease, or condition, are known as “appropriate for the disease, or condition, being treated.”

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically agent is a cancer agent (e.g., a biotherapeutic or chemo therapeutic cancer agent). In other embodiments, the additional therapeutically active agent is an anti-inflammatory agent.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### Articles of Manufacture and Kits

Also provided are articles of manufacture comprising any of the compounds or pharmaceutical compositions described herein. The articles of manufacture include suitable containers or packaging materials for the compounds or pharmaceutical compositions. Examples of a suitable container include, but are not limited to, a bottle, a vial, a syringe, an intravenous bag, or a tube.

Also provided are kits comprising any of the compounds or pharmaceutical compositions described herein. The kits can contain the compounds or pharmaceutical compositions in suitable containers or packaging materials, including, but not limited to, a bottle, a vial, a syringe, an intravenous bag, or a tube. The kits can comprise the compounds or pharmaceutical compositions for administration to an individual in single-dose form or in multiple-dose form. The kits can further comprise instructions or a label for administering the compounds or pharmaceutical compositions to an individual according to any of the methods disclosed herein. The kits can further comprise equipment for administering the compounds or pharmaceutical compositions to an individual, including, but not limited to, needles, syringes, tubing, or intravenous bags. The kits can further comprise instructions for producing any of the compounds or pharmaceutical compositions disclosed herein.

Also provided are articles of manufacture comprising any of the compounds, vaccines, or pharmaceutical compositions described herein. The articles of manufacture include suitable containers or packaging materials for the compounds or pharmaceutical compositions. The articles of manufacture include suitable containers or packaging materials for the compounds, oncolytic viruses, or pharmaceutical compositions. Examples of a suitable container include, but are not limited to, a bottle, a vial, a syringe, an intravenous bag, or a tube.

The disclosure will be more fully understood by reference to the following examples. They should not, however, be construed as limiting the scope of this disclosure. It is understood that the examples and embodiments described herein are for illustrative purposes only and that various modifications or changes in light thereof will be suggested to persons skilled in the art and are to be included within the spirit and purview of this application and scope of the appended claims.

### Analytical Methods and Instrumentation

Proton nuclear magnetic resonance (NMR) spectra were obtained on Bruker Ascend™ 500 MHz spectrometer. NMR spectra are reported as follows: chemical shift δ (ppm), multiplicity, coupling constant J (Hz), and integration. The abbreviations s=singlet, d=doublet, t=triplet, q=quartet, m=multiplet and br=broad are used throughout. Mass spectral data were measured using the following systems: Waters Acquity i-class ultra-performance liquid chromatography (UPLC) system with Acquity Photo Diode Array Detector, Acquity Evaporative Light Scattering Detector (ELSD) and Waters ZQ Mass Spectrometer. Data was acquired using Waters MassLynx 4.1 software and purity characterized by UV wavelength 220 nm, evaporative light scattering detection (ELSD) and electrospray positive ion (ESI) (column: Acquity UPLC BEH C18 1.7μ t 2.1×50 mm). Solvents used: acetonitrile/water, containing 0.1% formic acid; flow rate 0.7 mL/min. Preparatory HPLC purifications were conducted with a flow rate of 15 mL/min and detection by UV wavelength 214 nm and 254 nm (Column: Jupiter© 10 μM Proteo 90 Å, 250×21.2 mm A, solvent: acetonitrile/water, containing modifier such as 0.1% trifluoroacetic acid).”

Abbreviations used in the examples include:

| Abbreviation | Name |
|---|---|
| CH₃CN | acetonitrile |
| aq. | aqueous |
| atm | atmospheres |
| BINAP | (1,1′-binaphthalene-2,2′-diyl)bis(diphenylphosphine) |
| Boc | t-butoxycarbonyl |
| CCl₄ | carbon tetrachloride |
| CDCl₃ | deuterated chloroform |
| CO | carbon monoxide gas |
| CO₂ | carbon dioxide |
| Cs₂CO₃ | cesium carbonate |
| CuBr | copper(I) bromide |
| Cu(OAc)₂ | copper(II) acetate |
| DCM | dichloromethane |
| DEAD | diethyl azodicarboxylate |
| DIPEA | diisopropylethylamine |
| DMF | N,N-dimethylformamide |
| DMSO | dimethylsulfoxide |
| ESI | electrospray ionization |
| Et₃N | triethylamine |
| EtOAc | ethyl acetate |
| EtOH | ethanol |
| h | hours |
| H₂O | water |
| HATU | 1-[bis(dimethylamino)methylene]-1H-1,2,3-triazolo[4,5- |
| | b]pyridinium 3-oxide hexafluorophosphate |
| HCl | hydrogen chloride |
| HOAc | acetic acid |
| K₂CO₃ | potassium carbonate |
| KI | potassium iodide |
| KOH | potassium hydroxide |
| KOtBu | potassium tert-butoxide |
| LiAlH₄ | lithium aluminum hydride |
| LiOH | lithium hydroxide |
| MeOH | methanol |
| min | minutes |
| N₂ | nitrogen |
| Na₂CO₃ | sodium carbonate |
| Na₂SO₄ | sodium sulfate |
| NaH | sodium hydride |
| NaHCO₃ | sodium bicarbonate |
| NaOH | sodium hydroxide |
| NBS | N-bromosuccinimide |
| NH₄Cl | ammonium chloride |
| NMR | nuclear magnetic resonance |
| Pd₂(dba)₃ | tris(dibenzylideneacetone)dipalladium(0) |
| Pd(dppf)Cl₂ | [1,1′- |
| | bis(diphenylphosphino)ferrocene]dichloropalladium(II) |
| Pd/C | palladium on carbon |
| Pd(OAc)₂ | palladium(II) acetate |
| Prep-TLC | preparatory thin layer chromatography |
| RuPhos-Pd-G2 | chloro(2-dicyclohexylphosphino-2′,6′-diisopropoxy- |
| | 1,1′-biphenyl)[2-(2′-amino-1,1′-biphenyl)]palladium(II) |
| sat. | saturated |
| t-BuOH | tert-butanol |
| THF | tetrahydrofuran |

Additional embodiments are disclosed in further detail in the following examples, which are not in any way intended to limit the scope of the claims.

### General Schemes for Preparing LHP Building Blocks

CRBN-targeting LHM can be generally prepared according to Scheme B1-B3.

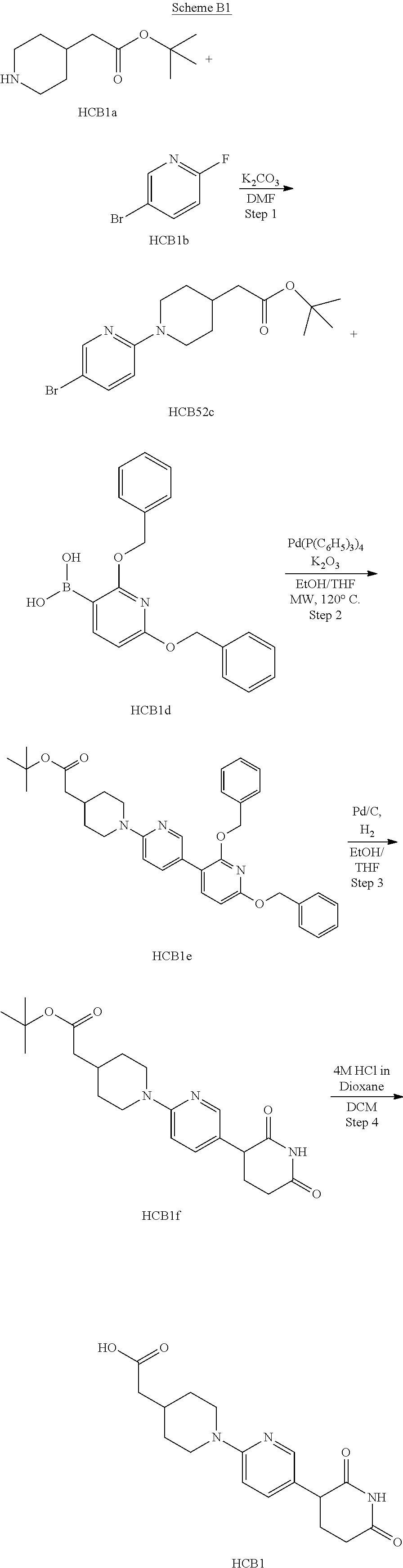

In certain embodiments, CRBN-targeting LHM building blocks comprising a carboxylic acid reactive group may be prepared according to Scheme B1. Described below are detail reaction procedures and additional examples of CRBN-targeting LHM building blocks that may be prepared according to Scheme B1.

### Example 1. {1-[5-(2,6-dioxopiperidin-3-yl)pyridin-2-yl]piperidin-4-yl}acetic acid (HCB1)

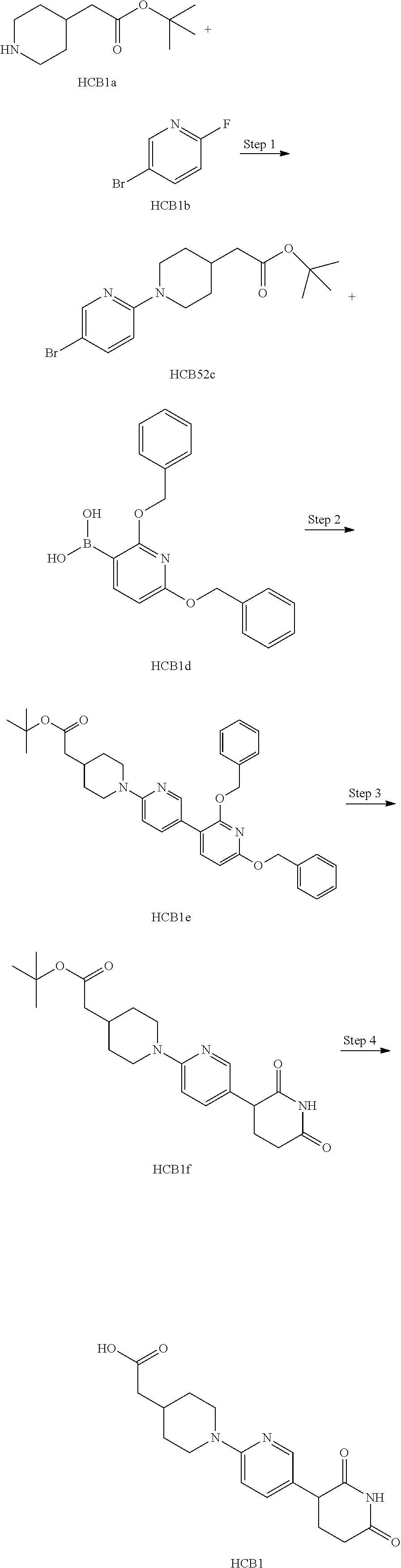

### Step 1: tert-butyl 2-[1-(5-bromopyridin-2-yl)piperidin-4-yl]acetate (HCB1c)

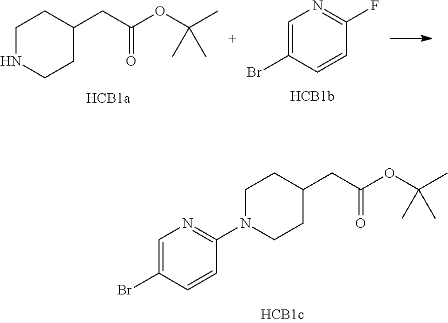 [SMILES: CC(C)(C)OC(=O)CC1CCNCC1 ; Fc1ccc(Br)cn1 ; CC(C)(C)OC(=O)CC1CCN(c2ccc(Br)cn2)CC1]

tert-butyl 2-(piperidin-4-yl)acetate (765 mg, 3.84 mmol), 5-bromo-2-fluoropyridine (0.40 mL, 676 mg, 3.84 mmol), and potassium carbonate (1.06 g, 7.68 mmol) were stirred in DMF (10.00 mL) overnight. Then the mixture was heated at 50° C. for six hours. The mixture was then stirred at room temperature for five days. The mixture was transferred to a separatory funnel with ethyl acetate and washed with two portions of water. The organic layer was dried over Na₂SO₄, filtered, and concentrated. The crude residue was purified by flash chromatography on a 40 g column (gradient elution with zero to 20% ethylacetate:hexanes) to provide tert-butyl 2-[1-(5-bromopyridin-2-yl)piperidin-4-yl]acetate (0.655 g, 48.0%). LCMS: C₁₆H₂₃BrN₂O₂ requires: 355, found: m/z=356 [M+H]⁺.

### Step 2: tert-butyl 2-{1-[2′,6′-bis(benzyloxy)-[3,3′-bipyridin]-6-yl]piperidin-4-yl}acetate (HCB1e)

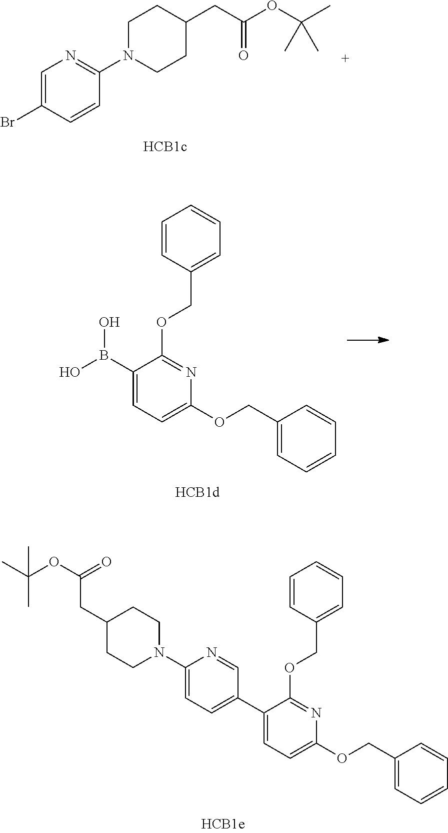

A mixture of 2,6-bis(benzyloxy)pyridin-3-ylboronic acid (495 mg, 1.48 mmol), tert-butyl 2-[1-(5-bromopyridin-2-yl)piperidin-4-yl]acetate (525 mg, 1.48 mmol), tetrakis(triphenylphosphine)palladium(0) (171 mg, 0.15 mmol), and potassium carbonate (408 mg, 2.96 mmol) in water (1.00 mL) and THF (3.00 mL) was microwaved at 120° C. for forty minutes. The water layer was removed by pipette. The organic layer was concentrated and then purified by flash chromatography on a 40 g column (gradient elution with zero to 35% ethyl acetate:hexanes) to provide tert-butyl 2-{1-[2′,6′-bis(benzyloxy)-[3,3′-bipyridin]-6-yl]piperidin-4-yl}acetate (0.303 g, 36.2%). LCMS: C₃₅H₃₉N₃O₄ requires: 565, found: m/z=566 [M+H]⁺.

### Step 3: tert-butyl 2-{1-[5-(2,6-dioxopiperidin-3-yl)pyridin-2-yl]piperidin-4-yl}acetate (HCB1f)

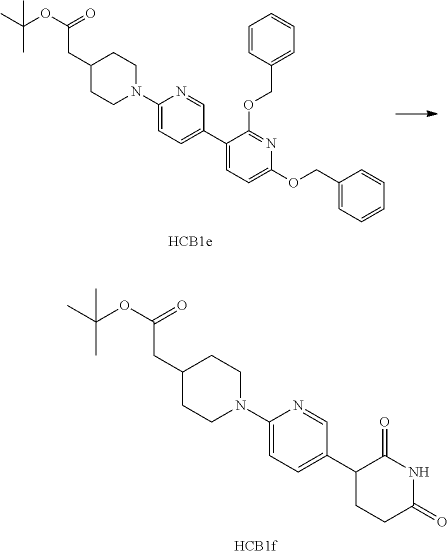 [SMILES: CC(C)(C)OC(=O)CC1CCN(c2ccc(-c3ccc(OCc4ccccc4)nc3OCc3ccccc3)cn2)CC1 ; CC(C)(C)OC(=O)CC1CCN(c2ccc(C3CCC(=O)NC3=O)cn2)CC1]

tert-butyl 2-{1-[2′,6′-bis(benzyloxy)-[3,3′-bipyridin]-6-yl]piperidin-4-yl}acetate (303 mg, 0.54 mmol) was suspended in EtOH (9.00 mL). THF (4 mL) was then added. 10% Pd/C (303 mg) was then added and the mixture was stirred under a balloon of H₂ for two hours. The mixture was diluted with THF (100 mL) and filtered through a pad of celite. The filtrate was concentrated. The crude residue was purified by flash chromatography on a 24 g column (gradient elution with zero to 10% MeOH:DCM) to provide tert-butyl 2-{1-[5-(2,6-dioxopiperidin-3-yl)pyridin-2-yl]piperidin-4-yl}acetate (0.152 g, 73.2%). LCMS: C₂₁H₂₉N₃O₄ requires: 387, found: m/z=388 [M+H]⁺.

### Step 4: {1-[5-(2,6-dioxopiperidin-3-yl)pyridin-2-yl]piperidin-4-yl}acetic acid (HCB1)

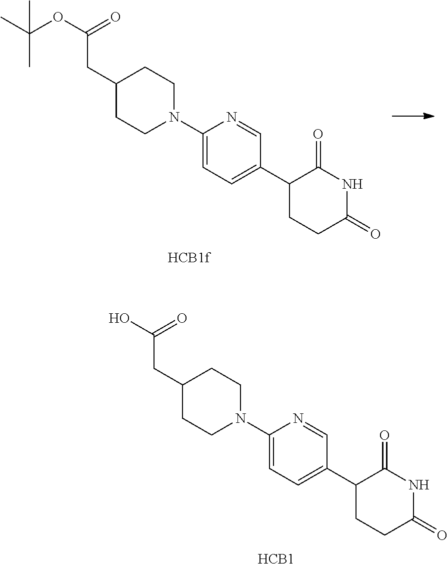 [SMILES: CC(C)(C)OC(=O)CC1CCN(c2ccc(C3CCC(=O)NC3=O)cn2)CC1 ; O=C(O)CC1CCN(c2ccc(C3CCC(=O)NC3=O)cn2)CC1 ; "HCB1"]

tert-butyl 2-{1-[5-(2,6-dioxopiperidin-3-yl)pyridin-2-yl]piperidin-4-yl}acetate (29.00 mg, 0.07 mmol) was stirred in DCM (0.50 mL) and 4 M HCl solution in dioxane (0.50 mL, 0.07 g, 2.00 mmol). After stirring overnight, the mixture was concentrated in vacuo to provide {1-[5-(2,6-dioxopiperidin-3-yl)pyridin-2-yl]piperidin-4-yl}acetic acid (0.0248 g, 100%). LCMS: C₁₇H₂₁N₃O₄ requires: 331, found: m/z=332 [M+H]⁺.

### Example 2. 1-(5-(2,6-dioxopiperidin-3-yl)pyridin-2-yl)piperidine-4-carboxylic acid (HCB2)

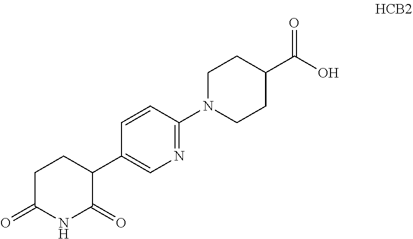 [SMILES: O=C1CCC(c2ccc(N3CCC(C(=O)O)CC3)nc2)C(=O)N1]

1-(5-(2,6-dioxopiperidin-3-yl)pyridin-2-yl)piperidine-4-carboxylic acid (HCB2) was prepared by procedures analogous to Example 1 starting from tert-butyl piperidine-4-carboxylate. LCMS: C₁₆H₁₉N₃O₄ requires: 317, found: m/z=318 [M+H]⁺.

### Example 3. 3-(6-(4-(hydroxymethyl)piperidin-1-yl)pyridin-3-yl)piperidine-2,6-dione (HCB4a)

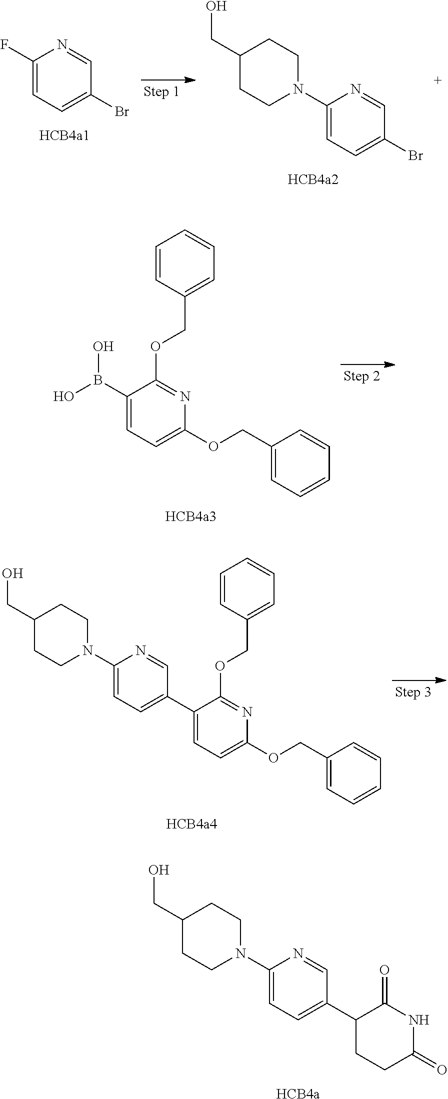 [SMILES: Fc1ccc(Br)cn1 ; OCC1CCN(c2ccc(Br)cn2)CC1 ; OB(O)c1ccc(OCc2ccccc2)nc1OCc1ccccc1 ; OCC1CCN(c2ccc(-c3ccc(OCc4ccccc4)nc3OCc3ccccc3)cn2)CC1 ; O=C1CCC(c2ccc(N3CCC(CO)CC3)nc2)C(=O)N1 ; "HCB4a"]

### Step 1: Synthesis of [1-(5-bromopyridin-2-yl)piperidin-4-yl]methanol (HCB4a2)

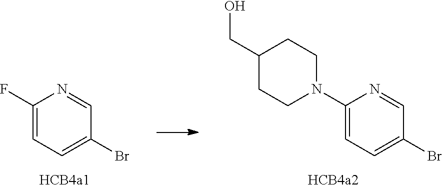 [SMILES: Fc1ccc(Br)cn1 ; OCC1CCN(c2ccc(Br)cn2)CC1]

To a mixture of 5-bromo-2-fluoropyridine (1.06 g, 6.00 mmol) in DMF (13.00 mL) was added piperidin-4-ylmethanol (691.10 mg, 6.00 mmol) and potassium carbonate (1.66 g, 12.00 mmol). The mixture was heated at 90° C. overnight. The mixture was partitioned between ethyl acetate and water, and the organic layer was dried over Na₂SO₄, filtered, and concentrated. The crude material was purified by flash chromatography on a 40 g column eluted with zero to 10% MeOH:DCM to provide [1-(5-bromopyridin-2-yl)piperidin-4-yl]methanol (829 mg, 51.7%). LCMS: C₁₁H₁₅BrN₂O requires 270, found: m/z=271 [M+H]⁺.

### Step 2: Synthesis of {1-[2′,6′-bis(benzyloxy)-[3,3′-bipyridin]-6-yl]piperidin-4-yl}methanol (HCB4a4)

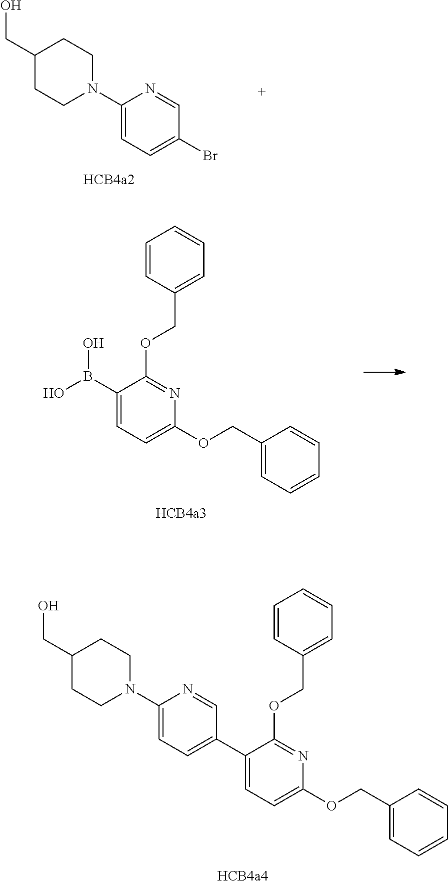 [SMILES: OCC1CCN(c2ccc(Br)cn2)CC1 ; "+" ; OB(O)c1ccc(OCc2ccccc2)nc1OCc1ccccc1 ; OCC1CCN(c2ccc(-c3ccc(OCc4ccccc4)nc3OCc3ccccc3)cn2)CC1]

[1-(5-bromopyridin-2-yl)piperidin-4-yl]methanol (282 mg, 1.04 mmol), 2,6-bis(benzyloxy)pyridin-3-ylboronic acid (523 mg, 1.56 mmol), tetrakis(triphenylphosphine)palladium(0) (120 mg, 0.10 mmol), and potassium carbonate (287 mg, 2.08 mmol) were deposited in a microwave vial in THF (3.00 mL) and water (1.00 mL). The mixture was microwaved at 120° C. for forty minutes. The organic layer was loaded directly onto a silica gel cartridge and the mixture was purified by flash chromatography on a 40 g column (gradient elution with zero to 50% EtOAc:DCM) to provide {1-[2′,6′-bis(benzyloxy)-[3,3′-bipyridin]-6-yl]piperidin-4-yl}methanol (0.198 g, 39.5%). LCMS: C₃₀H₃₁N₃O₃ requires 481, found: m/z=482 [M+H]⁺.

### Step 3: Synthesis of 3-(6-(4-(hydroxymethyl)piperidin-1-yl)pyridin-3-yl)piperidine-2,6-dione (HCB4a)

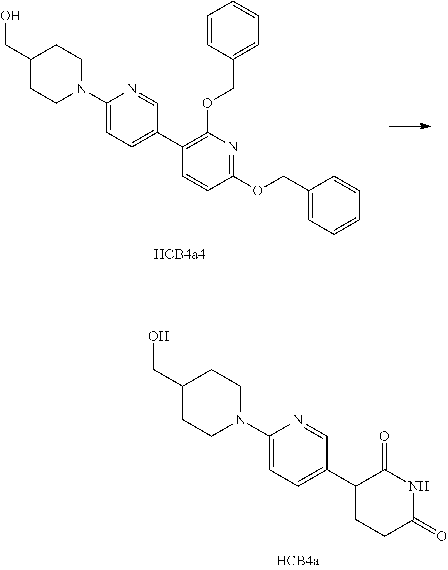 [SMILES: OCC1CCN(c2ccc(-c3ccc(OCc4ccccc4)nc3OCc3ccccc3)cn2)CC1 ; O=C1CCC(c2ccc(N3CCC(CO)CC3)nc2)C(=O)N1]

{1-[2′,6′-bis(benzyloxy)-[3,3′-bipyridin]-6-yl]piperidin-4-yl}methanol (198 mg, 0.41 mmol) and 10% Pd/C (198 mg) were stirred in EtOH (6.00 mL) under a balloon of H₂ for five hours. The mixture was diluted with THF and filtered through celite. The mixture was concentrated in vacuo and purified by flash chromatography on a 12 g column (gradient elution with zero to 20% MeOH:DCM) to provide 3-(6-(4-(hydroxymethyl)piperidin-1-yl)pyridin-3-yl)piperidine-2,6-dione (0.0339 g, 27.2%). LCMS: C₁₆H₂₁N₃O₃ requires 303, found: m/z=304 [M+H]⁺.

### Example 4. 3-(4-(4-(piperidin-4-ylmethyl)piperazin-1-yl)phenyl)piperidine-2,6-dione Synthesis (HCB35)

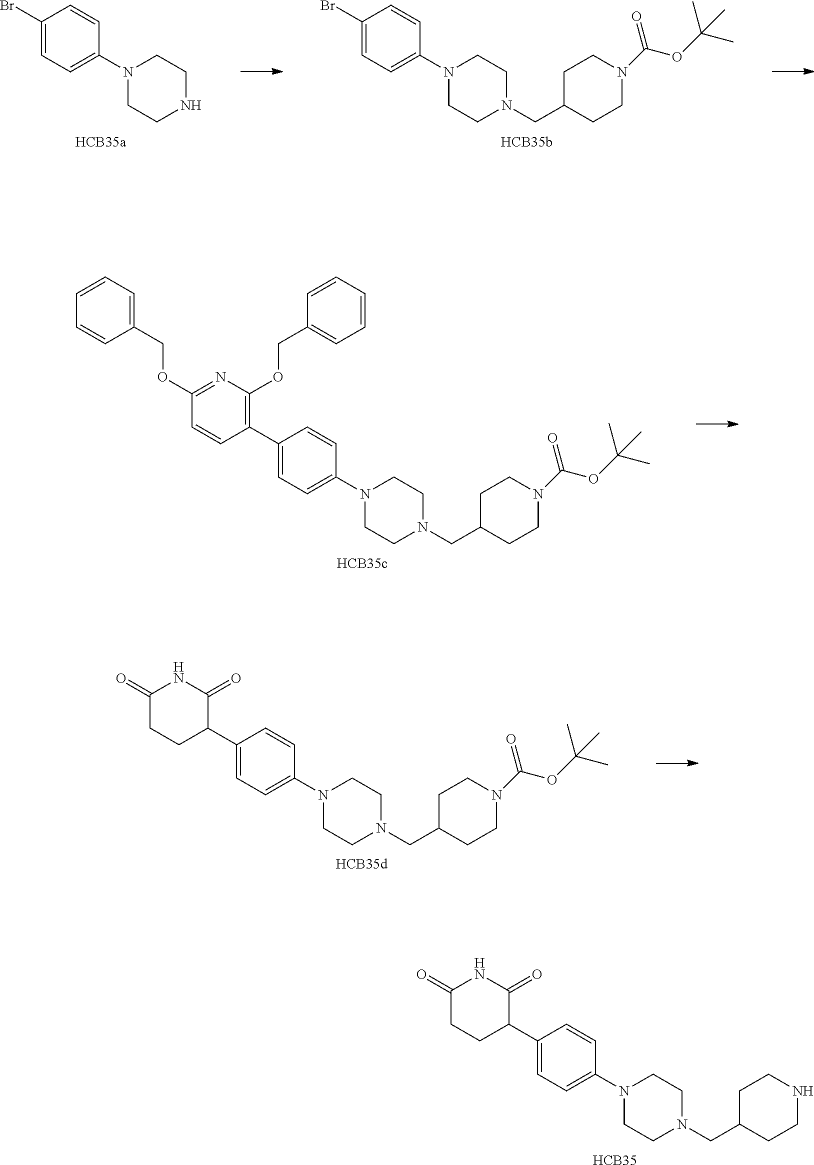

Step 1: To a solution of 1-(4-bromophenyl)piperazine (75.00 mg, 0.31 mmol) and tert-butyl 4-formylpiperidine-1-carboxylate (66.34 mg, 0.31 mmol) in DCM (2 mL) was added N,N-diisopropylethylamine (0.16 mL, 120.60 mg, 0.93 mmol), and sodium triacetoxyborohydride (396 mg, 1.86 mmol) as powder. The reaction was stirred for 30 mins when LCMS indicated completion of reaction. The crude product was dissolved in DCM (15 mL), washed with water, dried, and concentrated. Silica gel column purification (MeOH:DCM (2% TEA) 0-8% elution) provided the desired product (111 mg, 0.25 mmol, 81%). LCMS: C₂₁H₃₂BrN₃O₂ requires: 438, found: m/z=439 [M+H]⁺.

Step 2: To a solution of tert-butyl 4-{[4-(4-bromophenyl)piperazin-1-yl]methyl}piperidine-1-carboxylate (111 mg, 0.25 mmol) and tert-butyl 4-{[4-(4-bromophenyl)piperazin-1-yl]methyl}piperidine-1-carboxylate (85 mg, 0.25 mmol) in dioxane (2 mL) and water (0.5 mL) was added Cs₂CO₃ (247 mg, 0.76 mmol) and Pd(dppf)Cl₂.DCM (41 mg, 0.05 mmol). Nitrogen gas was bubbled through the reaction mixture and then reaction was heated at 100° C. for one hour when LCMS indicated reaction completion. The reaction mixture was dissolved in EtOAc, washed with brine, dried over Na₂SO₄, and concentrated. ISCO silica gel column purification eluting with MeOH:DCM (0-10%)provided the desired product (68 mg, 0.1 mmol, 41%). LCMS: C₄₀H₄₈N₄O₄ requires: 648.4, found: m/z=649.7 [M+H]⁺.

Step 3: tert-butyl 4-[(4-{4-[2,6-bis(benzyloxy)pyridin-3-yl]phenyl}piperazin-1-yl)methyl]piperidine-1-carboxylate (45 mg, 0.07 mmol) was added to a 25 mL round bottom flask. Then wet Pd on carbon (15 mg) and EtOH (3 mL) was added to dissolve starting material. The solution was stirred under a hydrogen balloon overnight. The reaction mixture was filtered through celite and the filtrate concentrated under reduced pressure. The crude product was purified by ISCO silica gel column chromatography eluting with MeOH:DCM having 2% TEA (0-12%)which provided the desired product. (18 mg, 0.04 mmol, 55%). LCMS: C₂₆H₂₈N₄O₄ requires: 470, found: m/z=471 [M+H]⁺.

Step 4: tert-butyl 4-({4-[4-(2,6-dioxopiperidin-3-yl)phenyl]piperazin-1-yl}methyl)piperidine-1-carboxylate (18 mg, 0.04 mmol) was dissolved in 4 N HCl in dioxane (2 mL). After 1 hr, the volatiles were removed and the residue was placed under vacuum to dryness to afford 3-{4-[4-(piperidin-4-ylmethyl)piperazin-1-yl]phenyl}piperidine-2,6-dione (quantitative yield). LCMS: C₂₁H₃₀N₄O₂ requires: 370, found: m/z=371 [M+H]⁺.

### Example 5. 3-(4-(4-(piperazin-1-ylmethyl)piperidin-1-yl)phenyl)piperidine-2,6-dione (HCB36)

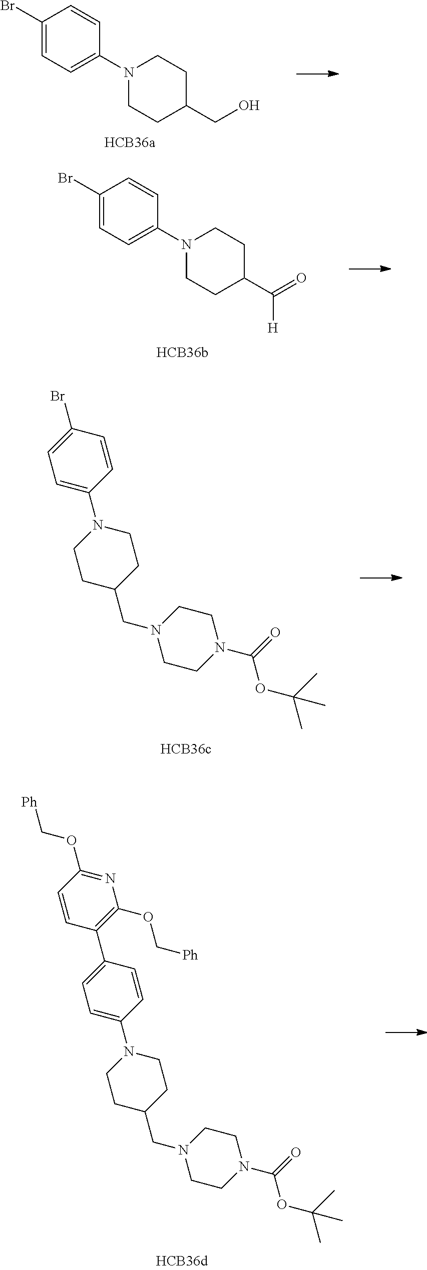 [SMILES: OCC1CCN(c2ccc(Br)cc2)CC1 ; O=CC1CCN(c2ccc(Br)cc2)CC1 ; CC(C)(C)OC(=O)N1CCN(CC2CCN(c3ccc(Br)cc3)CC2)CC1 ; CC(C)(C)OC(=O)N1CCN(CC2CCN(c3ccc(-c4ccc(OCc5ccccc5)nc4OCc4ccccc4)cc3)CC2)CC1]

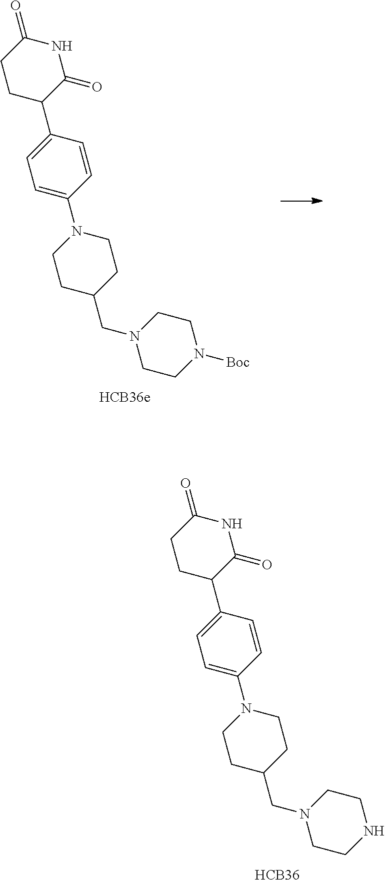 [SMILES: CC(C)(C)OC(=O)N1CCN(CC2CCN(c3ccc(C4CCC(=O)NC4=O)cc3)CC2)CC1 ; "HCB36" ; O=C1CCC(c2ccc(N3CCC(CN4CCNCC4)CC3)cc2)C(=O)N1]

Step 1: To a solution of [1-(4-bromophenyl)piperidin-4-yl]methanol (240.00 mg, 0.89 mmol) in DMSO (1.5 mL) was added triethylamine (2.50 mL, 1.80 g, 17.77 mmol) and then sulfur trioxide pyridine complex (1.41 g, 8.88 mmol). The reaction was stirred at rt for 30 mins when TLC showed no starting material left. The product was dissolved in EtOAc (50 mL) and the organic layer was washed water (2 mL×2). The organic layer was dried and concentrated. The crude material was used in the next step without purification.

Step 2: To a solution of 1-(4-bromophenyl)piperidine-4-carbaldehyde (230.00 mg, 0.86 mmol) and tert-butyl piperazine-1-carboxylate (0.16 g, 0.86 mmol) in DCM (10 mL) was added sodium triacetoxyborohydride (0.55 g, 0.257 mmol), and the reaction was stirred for 0.5 hr. The reaction mixture was quenched with 5% sodium bicarbonate solution and extracted with CH₂Cl₂. The organic layer was dried and concentrated under reduced pressure. Silica gel column purification eluting with 0-80% EtOAc in hexane provided the desired product (221 mg, 0.5 mmol, 59%). LCMS: C₂₁H₃₂BrN₃O₂ requires: 437.2, found: m/z=439 [M+H]⁺.

Step 3: tert-butyl 4-[(1-{4-[2,6-bis(benzyloxy)pyridin-3-yl]phenyl}piperidin-4-yl)methyl]piperazine-1-carboxylate. The compound was prepared following the same reaction procedure as in Step 2 of Example 38. LCMS: C₄₀H₄₈N₄O₄ requires: 648.4, found: m/z=649.7

Step 4: tert-butyl 4-((1-(4-(2,6-dioxopiperidin-3-yl)phenyl)piperidin-4-yl)methyl)piperazine-1-carboxylate. The compound was prepared following the same reaction procedure as in Step 2 of Example 38. LCMS: C₂₆H₃₈N₄O₄ requires: 470.3, found: m/z=471.6 [M+H]⁺.

Step 5: 3-(4-(4-(piperazin-1-ylmethyl)piperidin-1-yl)phenyl)piperidine-2,6-dione. The compound was prepared following the same reaction procedure as in Step 2 of Example 38. LCMS: C₂₁H₃₀N₄O₂ requires: 370.2, found: m/z=371.3 [M+H]⁺.

### Example 6. 3-(5-(4-(piperidin-4-ylmethyl)piperazin-1-yl)pyridin-2-yl)piperidine-2,6-dione (HCB37)

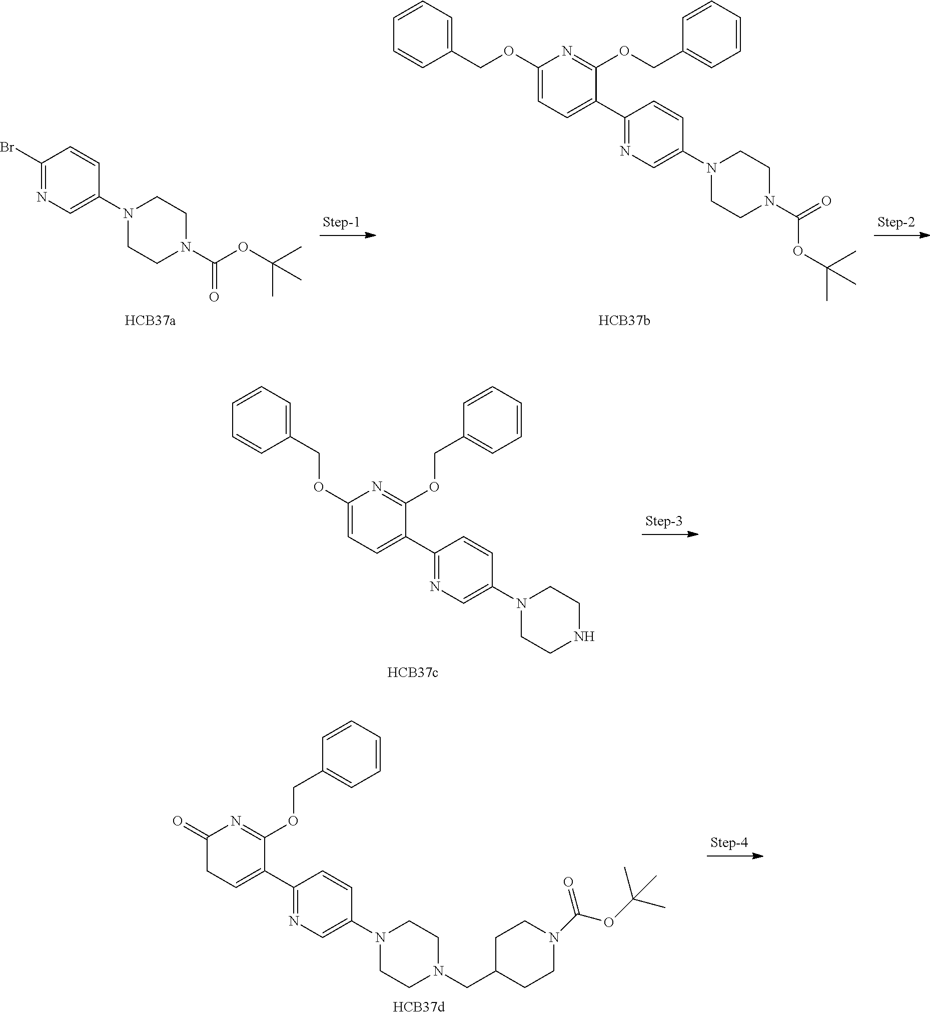 [SMILES: CC(C)(C)OC(=O)N1CCN(c2ccc(Br)nc2)CC1 ; CC(C)(C)OC(=O)N1CCN(c2ccc(-c3ccc(OCc4ccccc4)nc3OCc3ccccc3)nc2)CC1 ; c1ccc(COc2ccc(-c3ccc(N4CCNCC4)cn3)c(OCc3ccccc3)n2)cc1 ; CC(C)(C)OC(=O)N1CCC(CN2CCN(c3ccc(C4=CCC(=O)N=C4OCc4ccccc4)nc3)CC2)CC1]

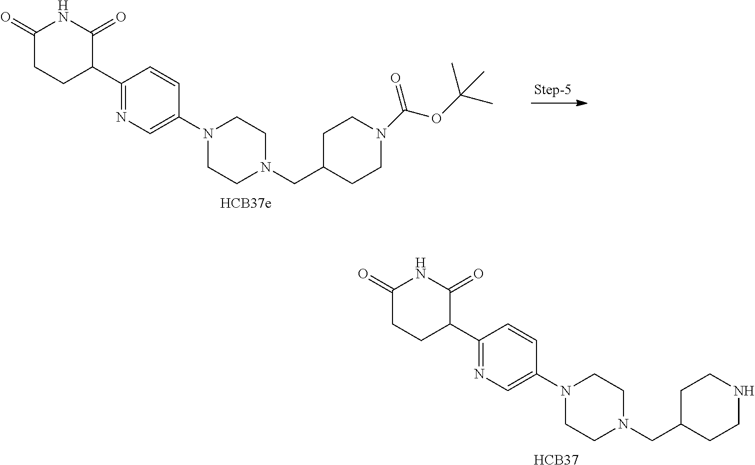

Step 1: A solution of tert-butyl 4-(6-bromopyridin-3-yl)piperazine-1-carboxylate (250.00 mg, 0.73 mmol), 2,6-bis(benzyloxy)pyridin-3-ylboronic acid (244.84 mg, 0.73 mmol), cesium carbonate (714.02 mg, 2.19 mmol), and Pd(dppf)Cl₂.DCM in dioxane (4 mL) and water (1 mL) was heated at 100° C. for two hours. The reaction mixture was cooled, dissolved in EtOAc (30 mL), and washed with brine. The organic layer was dried over sodium sulfate and concentrated. ISCO silica gel column purification eluting with EtOAc in hexane (5-100%) provided tert-butyl 4-[2′,6′-bis(benzyloxy)-[2,3′-bipyridin]-5-yl]piperazine-1-carboxylate (168 mg, 41%). LCMS: C₃₃H₃₆N₄O₄ requires: 552.3, found: m/z=553.7 [M+H]⁺.

Step 2: tert-butyl 4-[2′,6′-bis(benzyloxy)-[2,3′-bipyridin]-5-yl]piperazine-1-carboxylate (80.00 mg, 0.14 mmol) was dissolved in 4 N HCl in dioxane (4 mL) and the reaction was left to stand for one hour. The volatiles were removed and the crude material was placed under vacuum until dryness to provide 2′,6′-bis(benzyloxy)-5-(piperazin-1-yl)-2,3′-bipyridine HCl salt (68 mg, quantitative yield).

Step 3: To a solution of 2′,6′-bis(benzyloxy)-5-(piperazin-1-yl)-2,3′-bipyridine (60.00 mg, 0.13 mmol) and tert-butyl 4-formylpiperidine-1-carboxylate (28.28 mg, 0.13 mmol) in DCM (1 mL) and DMSO (0.3 mL) as added N,N-diisopropylethylamine (23.16 μL, 0.02 g, 0.13 mmol) and sodium triacetoxyborohydride (84.30 mg, 0.40 mmol). The reaction was stirred for 30 minutes when LCMS showed no starting material remained. The crude was dissolved in DCM (30 mL), washed with water, dried over Na₂SO₄, and concentrated. The crude product was purified by silica gel column chromatography eluting with MeOH in DCM having 2% TEA (0-8%) which provided tert-butyl 4-({4-[2′-(benzyloxy)-6′-oxo-5′H-[2,3′-bipyridin]-5-yl]piperazin-1-yl}methyl)piperidine-1-carboxylate (32 mg, 43%). LCMS: C₃₉H₄₇N₅O₄ requires: 559.3, found: m/z=560.7 [M+H]⁺.

Step 4: A suspension of tert-butyl 4-({4-[2′-(benzyloxy)-6′-oxo-5′H-[2,3′-bipyridin]-5-yl]piperazin-1-yl}methyl)piperidine-1-carboxylate (32 mg, 0.06 mmol) and 10% Pd/C (10 mg) in ethanol (5 mL) was vigorously stirred under a H₂ atmosphere (balloon) for sixteen hours. The solids were filtered, washed with DCM, and the volatiles were removed. The crude product was purified by silica gel column chromatography eluting with MeOH in DCM with 2% TEA (0-10%) which provided tert-butyl 4-((4-(6-(2,6-dioxopiperidin-3-yl)pyridin-3-yl)piperazin-1-yl)methyl)piperidine-1-carboxylate (28 mg, 92%). LCMS: C₂₅H₃₇N₅O₄ requires: 471.6, found: m/z=472.5 [M+H]⁺.

Step 5: tert-butyl 4-((4-(6-(2,6-dioxopiperidin-3-yl)pyridin-3-yl)piperazin-1-yl)methyl)piperidine-1-carboxylate (28 mg, 0.06 mmol) was dissolved in 4 N HCl in dioxane (4 mL) and the reaction was left to stand for one hour. The volatiles were removed and the material was placed under vacuum until dryness to provide 3-(5-(4-(piperidin-4-ylmethyl)piperazin-1-yl)pyridin-2-yl)piperidine-2,6-dione (quantitative yield). LCMS: C₂₀H₂₉N₅O₂ requires: 371.2, found: m/z=372.5 [M+H]⁺.

### Example 7. 3-(5-(4-(piperazin-1-ylmethyl)piperidin-1-yl)pyridin-2-yl)piperidine-2,6-dione synthesis (HCB38)

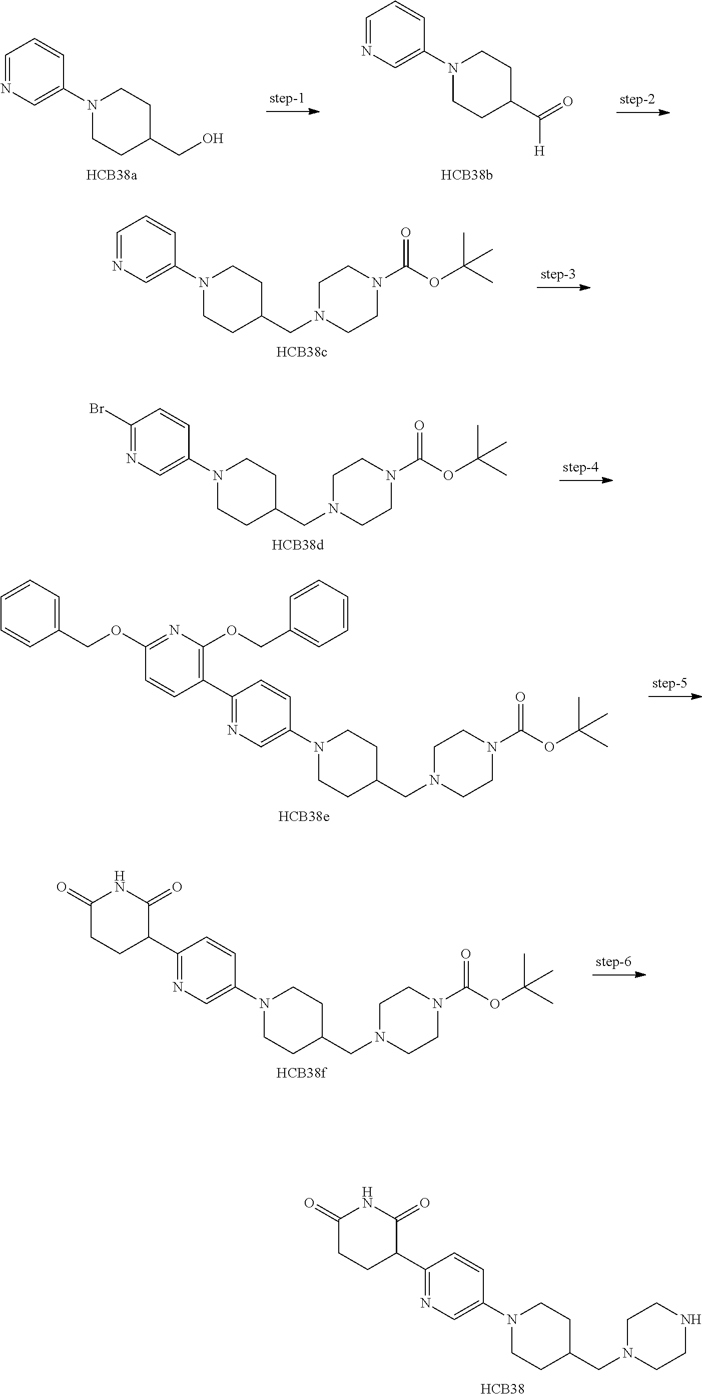

Step 1: To a solution of [1-(pyridin-3-yl)piperidin-4-yl]methanol (300.00 mg, 1.56 mmol) in DMSO (2 mL) was added triethylamine (4.39 mL, 3.16 g, 31.21 mmol), and then sulfur trioxide pyridine complex (2.48 g, 15.60 mmol, powder) was added slowly. The reaction was stirred at rt for 30 minutes when TLC showed no starting material remained. The product was dissolved in EtOAc (50 mL) and the organic solution was washed with water (3 mL×2). The organic layer was dried over Na₂SO₄, filtered, and concentrated to provide 1-(pyridin-3-yl)piperidine-4-carbaldehyde (0.2800 g, 94.3%) as crude product. The crude product was used in the next step without purification.

Step 2: To a solution of 1-(pyridin-3-yl)piperidine-4-carbaldehyde (280.00 mg, 1.47 mmol) and tert-butyl piperazine-1-carboxylate (0.27 g, 1.47 mmol) in DCM (10 mL) was added sodium triacetoxyborohydride (0.94 g, 4.42 mmol) and the reaction was stirred for 30 minutes. The reaction was quenched with sodium bicarbonate solution (2 mL), and the mixture was extracted DCM (50 mL). The organic layer was dried over Na₂SO₄, filtered, and concentrated. Silica gel column purification eluting with 0-10% MeOH:DCM provided the desired product (403 mg, 1.12 mmol, 76%). LCMS: C₂₀H₃₂N₄O₂ requires: 360.3, found: m/z=361.6 [M+H]⁺. ¹E1 NMR (500 MHz, CDCl₃) δ 8.34 (d, J=2.9 Hz, 1H), 8.09 (dd, J=4.6, 1.4 Hz, 1H), 7.23-7.20 (m, 1H), 7.16 (dd, J=8.5, 4.5 Hz, 1H), 3.72 (d, J=12.4 Hz, 2H), 3.45 (t, J=5.0 Hz, 4H), 2.77 (td, J=12.1, 2.6 Hz, 2H), 2.64 (s, 3H), 2.38 (d, J=5.5 Hz, 5H), 2.25 (d, J=7.1 Hz, 2H), 1.91 (d, J=13.2 Hz, 2H), 1.70 (ddd, J=11.2, 7.5, 3.8 Hz, 1H), 1.49 (s, 9H), 1.36 (qd, J=12.2, 4.1 Hz, 2H).

Step 3: To a solution of tert-butyl 4-{[1-(pyridin-3-yl)piperidin-4-yl]methyl}piperazine-1-carboxylate (403.00 mg, 1.12 mmol) in MeCN (10 mL) was slowly added NBS (238 mg, 1.34 mmol) in MeCN (1 mL) and the reaction was stirred at rt for one hour. The reaction was monitored by LCMS which showed no starting material remained. The volatiles were removed and the crude product was purified by silica gel column chromatography eluting with EtOAc:DCM (0-100%) to afford desired product (242 mg, 0.55 mmol, 49%). LCMS: C₂₀H₃₁BrN₄O₂ requires: 439.2, found: m/z=441.3 [M+H]⁺. ¹H NMR (500 MHz, CDCl₃) δ 8.03 (d, J=3.2 Hz, 1H), 7.30 (d, J=8.1 Hz, 1H), 7.10 (dd, J=8.8, 3.2 Hz, 1H), 3.67 (dt, J=13.0, 3.4 Hz, 2H), 3.45 (t, J=5.1 Hz, 4H), 2.85-2.69 (m, 2H), 2.37 (d, J=5.2 Hz, 4H), 2.24 (d, J=7.2 Hz, 2H), 1.90 (d, J=13.2 Hz, 2H), 1.69 (ddt, J=11.2, 7.6, 3.7 Hz, 1H), 1.49 (s, 9H), 1.42-1.27 (m, 2H).

Step 4: A solution of tert-butyl 4-{[1-(6-bromopyridin-3-yl)piperidin-4-yl]methyl}piperazine-1-carboxylate (242.00 mg, 0.55 mmol) and 2,6-bis(benzyloxy)pyridin-3-ylboronic acid (184 mg, 0.55 mmol) in dioxane (5 mL) and water (1.25 mL) was added cesium carbonate (714.02 mg, 2.19 mmol) and Pd(dppf)Cl₂.DCM. Nitrogen was bubbled through the reaction for five minutes and then the reaction was heated at 100° C. for two hours. The reaction was cooled, diluted with EtOAc (40 mL), and washed with brine. The organic layer was dried over Na₂SO₄, filtered, and concentrated. ISCO silica gel column purification eluting with EtOAc in DCM (20-100%) provided tert-butyl 4-({1-[2′,6′-bis(benzyloxy)-[2,3′-bipyridin]-5-yl]piperidin-4-yl}methyl)piperazine-1-carboxylate (238 mg, 0.37 mmol, 67%). LCMS: C₃₉H₄₇N₅O₄ requires: 649.4, found: m/z=650.8 [M+H]⁺.

Step 5: A suspension of tert-butyl 4-({1-[2′,6′-bis(benzyloxy)-[2,3′-bipyridin]-5-yl]piperidin-4-yl}methyl)piperazine-1-carboxylate (238 mg, 0.37 mmol) and Pd on carbon (90 mg) in ethanol (8 mL) and THF (4 mL) was stirred under hydrogen gas overnight. The solids were filtered and the volatiles were removed. Silica gel column purification eluting with MeOH:DCM (0-8%), provided the desired product (46 mg, 0.1 mmol, 27%). LCMS: C₂₅H₃₇N₅O₄ requires: 471.3, found: m/z=472.5 [M+H]⁺.

Step 6: tert-butyl 4-[(1-{6-[(3R)-2,6-dioxopiperidin-3-yl]pyridin-3-yl}piperidin-4-yl)methyl]piperazine-1-carboxylate was dissolved in DCM (1 mL). 4 N HCl in dioxane (1 mL) was added and the reaction was left to stand for one hour. After the volatiles were removed the desired product was obtained (quantitative yield). LCMS: C₂₀H₂₉N₅O₂ requires: 371.2, found: m/z=372.4 [M+H]⁺.

### Example 8. 3-(6-(4-(piperidin-4-ylmethyl)piperazin-1-yl)pyridin-2-yl)piperidine-2,6-dione (HCB39)

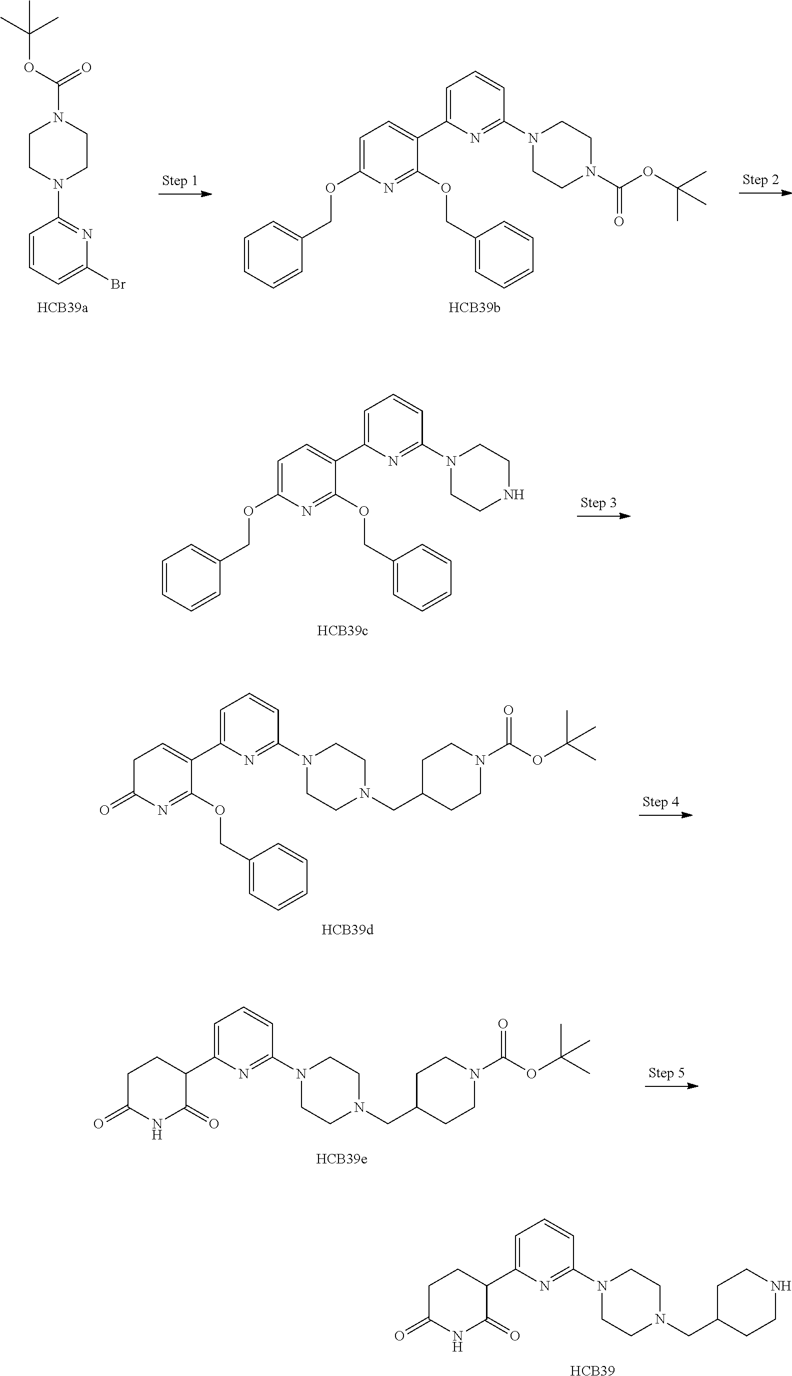

Step 1: The HBC38 Step 4 procedure was adapted to provide tert-butyl 4-(2′,6′-bis(benzyloxy)-[2,3′-bipyridin]-6-yl)piperazine-1-carboxylate. LCMS: C₃₃H₃₆N₄O₄ requires: 552.3, found: m/z=553.5 [M+H]⁺.

Step 2: The HBC38 Step 6 procedure was adapted to provide 2′,6′-bis(benzyloxy)-6-(piperazin-1-yl)-2,3′-bipyridine. LCMS: C₂₈H₂₈N₄O₂ requires: 452.2, found: m/z=453.5 [M+H]⁺.

Step 3: 2′,6′-bis(benzyloxy)-6-(piperazin-1-yl)-2,3′-bipyridine (96.00 mg, 0.21 mmol) and tert-butyl 4-formylpiperidine-1-carboxylate (45.24 mg, 0.21 mmol) were dissolved in 4 ml DCM, sodium triacetoxyborohydride (179.8 mg, 0.85 mmol) was added, stirred for 30 mins. The crude was dissolved in DCM (30 mL), washed with water, dried over Na₂SO₄, and concentrated. The crude product was purified by silica gel column chromatography eluting with 0-80% EtOAc/Hexane, obtained tert-butyl 4-((4-(2′-(benzyloxy)-6′-oxo-5′,6′-dihydro-[2,3′-bipyridin]-6-yl)piperazin-1-yl)methyl)piperidine-1-carboxylate (76 mg, 0.14 mmol, 64%). LCMS: C₃₂H₄₁N₅O₄ requires: 559.3, found: m/z=560.8 [M+H]⁺.

Step 4: A suspension of tert-butyl 4-((4-(2′-(benzyloxy)-6′-oxo-5′,6′-dihydro-[2,3′-bipyridin]-6-yl)piperazin-1-yl)methyl)piperidine-1-carboxylate (76 mg, 0.14 mmol) and Pd on carbon (22 mg) in ethanol (3 mL) and THF (3 mL) was stirred under hydrogen gas overnight. The solids were filtered and the volatiles were removed. Silica gel column purification eluting with EtOAc, provided the desired product (34 mg, 0.07 mmol, 53%). tert-butyl 4-((4-(2′-(benzyloxy)-6′-oxo-5′,6′-dihydro-[2,3′-bipyridin]-6-yl)piperazin-1-yl)methyl)piperidine-1-carboxylate. LCMS: C₂₅H₃₇N₅O₄ requires: 471.3, found: m/z=472.6 [M+H]⁺.

Step 5: tert-butyl 4-((4-(2′-(benzyloxy)-6′-oxo-5′,6′-dihydro-[2,3′-bipyridin]-6-yl)piperazin-1-yl)methyl)piperidine-1-carboxylate (34 mg, 0.07 mmol) in DCM (1 mL). 4 N HCl in dioxane (2 mL) was added and the reaction was left to stand for one hour. After the volatiles were removed the desired product was obtained (quantitative yield). 3-(6-(4-(piperidin-4-ylmethyl)piperazin-1-yl)pyridin-2-yl)piperidine-2,6-dione. LCMS: C₂₀H₂₉N₅O₂ requires: 371.2, found: m/z=372.5 [M+H]⁺.

### Example 9. 3-(6-(4-((methyl(piperidin-4-yl)amino)methyl)piperidin-1-yl)pyridin-3-yl)piperidine-2,6-dione (HCB41)

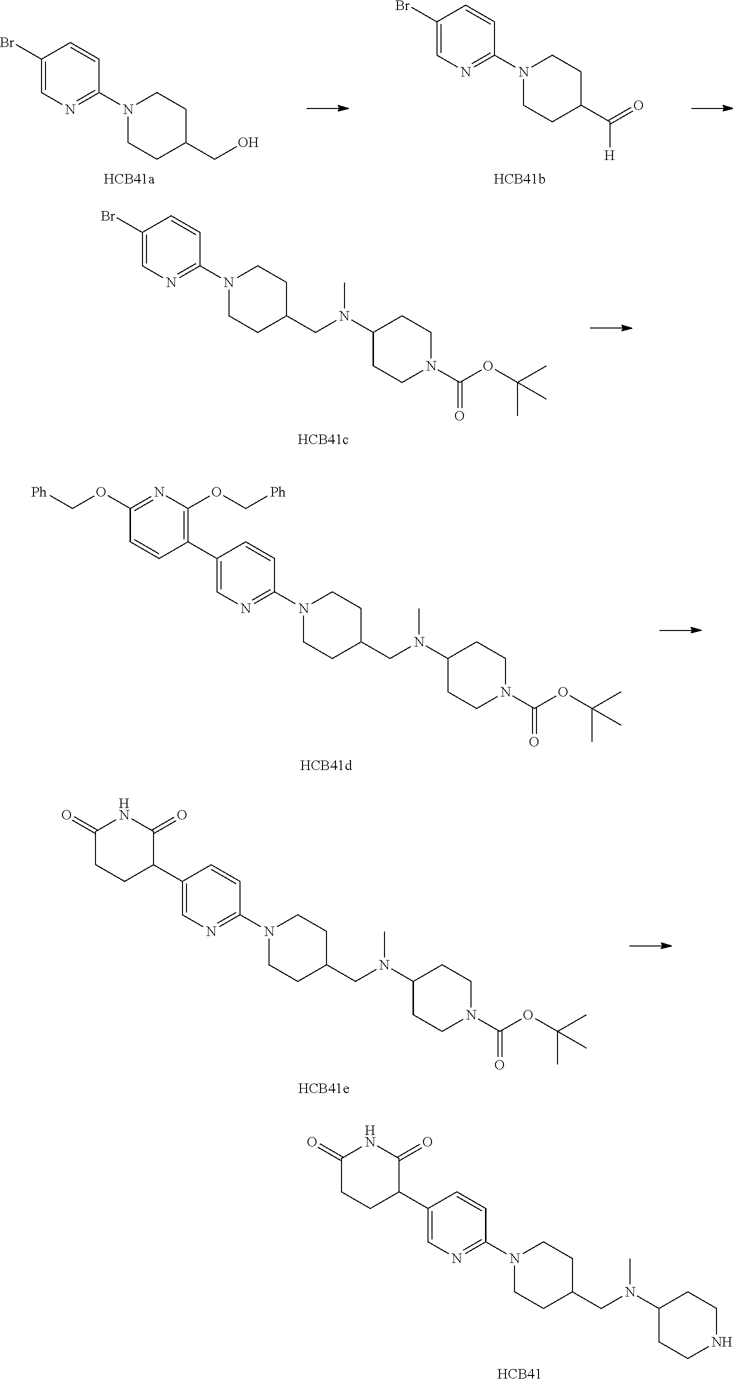

Step 1 and 2: [1-(5-bromopyridin-2-yl)piperidin-4-yl]methanol (150 mg, 0.55 mmol) was dissolved in DCM (4 mL) and DMSO (1 mL), and then triethylamine (0.77 mL, 5.53 mmol) and sulfur trioxide pyridine complex (440 mg, 2.77 mmol) was added. The reaction was stirred for 30 min when TLC indicated no more starting material remained. The reaction mixture was diluted with DCM (40 mL), and the organic solution was washed with water (2 mL×2). The organic solution was dried over sodium sulfate, concentrated, and the crude product was then re-dissolved in DCM (10 mL). Tert-butyl 4-(methylamino)piperidine-1-carboxylate (119 mg, 0.55 mmol) and sodium triacetoxyborohydride (0.3 g, 1.5 mmol) was added, and the reaction was stirred for thirty minutes. The reaction was quenched with sodium bicarbonate solution (2 mL) and extracted with DCM (50 mL). The organic layer was dried over Na₂SO₄, filtered, and concentrated. Silica gel column purification eluting with 0-6% MeOH in DCM provided the desired product (75 mg, 0.16 mmol, 29% over two steps). LCMS: C₂₂H₃₅BrN₄O₂ requires: 467, found: m/z=469 [M+H]⁺.

Step 3: 2,6-bis(benzyloxy)pyridin-3-ylboronic acid (52.34 mg, 0.16 mmol), tert-butyl 4-({[1-(5-bromopyridin-2-yl)piperidin-4-yl]methyl}(methyl)amino)piperidine-1-carboxylate (73.00 mg, 0.16 mmol) and cesium carbonate (152.65 mg, 0.47 mmol), Pd(dppf)C₁₂-DCM (25.51 mg, 0.03 mmol) were dissolved in 2 ml dioxane and 0.5 ml water, bubbled nitrogen gas for 3 minutes. The reaction mixture was heated at 100° C. for 1 hour. LCMS showed no starting material left. Evaporated solvent under reduced pressure. Dissolved product in 30 ml EtOAc, washed with brine, dried, concentrated. ISCO silica gel column purification eluting with MeOH/DCM (0-10%), isolated tert-butyl 4-[({1-[2′,6′-bis(benzyloxy)-[3,3′-bipyridin]-6-yl]piperidin-4-yl}methyl)(methyl)amino]piperidine-1-carboxylate carboxylate (68 mg, 64%). LCMS: C₄₁H₅₁N₅O₄ requires: 677.4, found: m/z=678.6 [M+H]⁺.

Step 4: The suspension of tert-butyl 4-[({1-[2′,6′-bis(benzyloxy)-[3,3′-bipyridin]-6-yl]piperidin-4-yl}methyl)(methyl)amino]piperidine-1-carboxylate (68.00 mg, 0.10 mmol) and 20 mg palladium on carbon (wet) in 5 ml ethanol was stirred under hydrogen gas overnight. The solids were filtered and the volatiles were removed. Silica gel column purification eluting with MeOH:DCM with 2% TEA) (0-10%), provided the desired product (28 mg, 0.06 mmol, 56%). tert-butyl 4-[({1-[5-(2,6-dioxopiperidin-3-yl)pyridin-2-yl]piperidin-4-yl}methyl)(methyl)amino]piperidine-1-carboxylate. LCMS: C₂₇H₄₁N₅O₄ requires: 499.3, found: m/z=500.5 [M+H]⁺.

Step 5: 4-[({1-[5-(2,6-dioxopiperidin-3-yl)pyridin-2-yl]piperidin-4-yl}methyl)(methyl)amino]piperidine-1-carboxylate (28 mg, 0.06 mmol) was dissolved in DCM (1 mL). 4 N HCl in dioxane (1 mL) was added and the reaction was left to stand for one hour. After the volatiles were removed the desired product was obtained (quantitative yield). 3-(6-(4-((methyl(piperidin-4-yl)amino)methyl)piperidin-1-yl)pyridin-3-yl)piperidine-2,6-dione. LCMS: C₂₂H₃₃N₅O₄ requires: 399.3, found: m/z=400.5 [M+H]⁺.

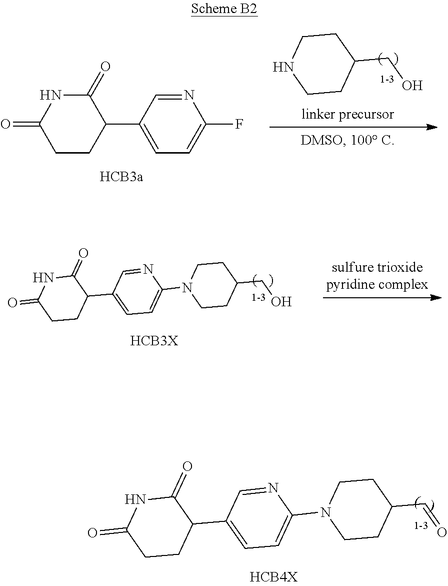

In certain embodiments, CRBN-targeting LHM building blocks comprising alcohol and aldehyde reactive groups that may be prepared according to Scheme B2.

In Scheme B2, a functionalized dione was first coupled to a linker precursor in step 1 to furnish harness building block (HCB3X). The harness building block from step 1 was subjected to oxidation reaction conditions to provide new harness building block (HCB4X). Described below are detail reaction procedures and additional examples of CRBN-targeting LHM building blocks that may be prepared according to Scheme B2.

### Example 10. 3-(6-(4-(2-hydroxyethyl)piperidin-1-yl)pyridin-3-yl)piperidine-2,6-dione (HCB3)

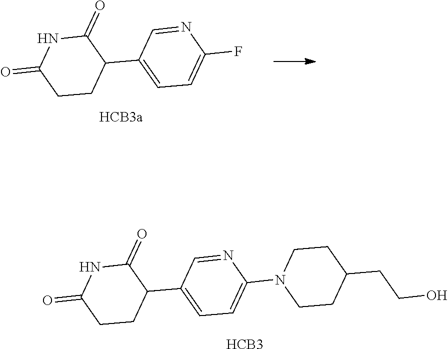

A mixture of 3-(6-fluoropyridin-3-yl)piperidine-2,6-dione (43.4 mg, 0.21 mmol), 4-piperidineethanol (26.9 mg, 0.21 mmol), and N,N-diisopropylethylamine (0.07 mL, 0.05 g, 0.42 mmol) in DMSO (1.00 mL) was heated in a 100° C. heating block for two days. Water was added and the mixture was extracted three times with ethyl acetate. The combined organic layers were concentrated. The crude residue was purified by preparative TLC in 5% MeOH:DCM to provide 3-(6-(4-(2-hydroxyethyl)piperidin-1-yl)pyridin-3-yl)piperidine-2,6-dione (0.0149 g, 22.5%). LCMS: C₁₇H₂₃N₃O₃ requires: 317, found: m/z=318 [M+H]⁺.

### Example 11. 3-(6-(4-(piperidin-4-ylmethyl)piperazin-1-yl)pyridin-3-yl)piperidine-2,6-dione (HCB10)

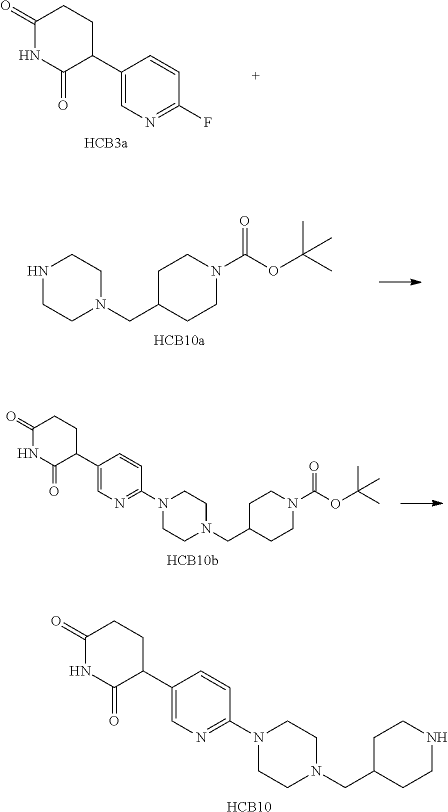

Step -1: A solution of 3-(6-fluoropyridin-3-yl)piperidine-2,6-dione (40 mg, 0.19 mmol), tert-butyl 4-(piperazin-1-ylmethyl)piperidine-1-carboxylate (54 mg, 0.19 mmol) and i-Pr2NEt (100 μL, 0.58 mmol) in NMP (0.47 mL) was stirred at 120° C. for 16 h. The reaction mixture was diluted with EtOAc, washed with H₂O, and concentrated under reduced pressure. Flash chromatography (SiO₂, 0-100% MeOH-DCM gradient elution) to provide the desired compound (54 mg, 0.11 mmol, 60%). LCMS: C₂₅H₃₇N₅O₄ requires: 471, found: m/z=472 [M+H]⁺.

Step 2: HCl (4M in dioxane, 198 μL, 0.77 mmol) was added to a solution of tert-butyl 4-((4-(5-(2,6-dioxopiperidin-3-yl)pyridin-2-yl)piperazin-1-yl)methyl)piperidine-1-carboxylate (36 mg, 0.08 mmol) in dioxane (0.9 mL). After stirred for 16 h, a reaction mixture was concentrated under reduced pressure to provide the desired compound. LCMS: C₂₀H₂₉N₅O₂ requires: 371, found: m/z=372 [M+H]⁺.

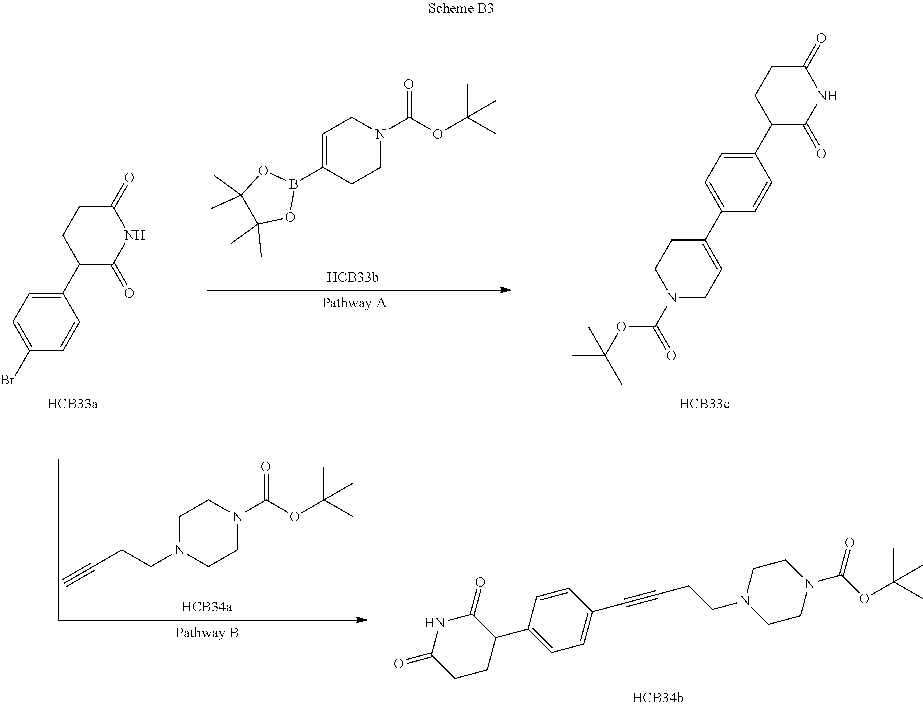 [SMILES: O=C1CCC(c2ccc(Br)cc2)C(=O)N1 ; CC(C)(C)OC(=O)N1CC=C(B2OC(C)(C)C(C)(C)O2)CC1 ; CC(C)(C)OC(=O)N1CC=C(c2ccc(C3CCC(=O)NC3=O)cc2)CC1 ; C#CCCN1CCN(C(=O)OC(C)(C)C)CC1 ; CC(C)(C)OC(=O)N1CCN(CCC#Cc2ccc(C3CCC(=O)NC3=O)cc2)CC1]

In certain embodiments, CRBN-targeting LHM building blocks comprise certain moieties that may be prepared using cross-coupling reaction conditions according to pathway A of Scheme B3. In certain embodiments, CRBN-targeting LHM building blocks comprising an alkyne reactive moiety may be prepared using cross-coupling reaction conditions according to pathway B of Scheme B3. Described below are detail reaction procedures and additional examples of CRBN-targeting LHM building blocks that may be prepared according to Scheme B3.

### Example 12. 3-(4-(1-(piperidin-4-ylmethyl)piperidin-4-yl)phenyl)piperidine-2,6-dione (HCB33)

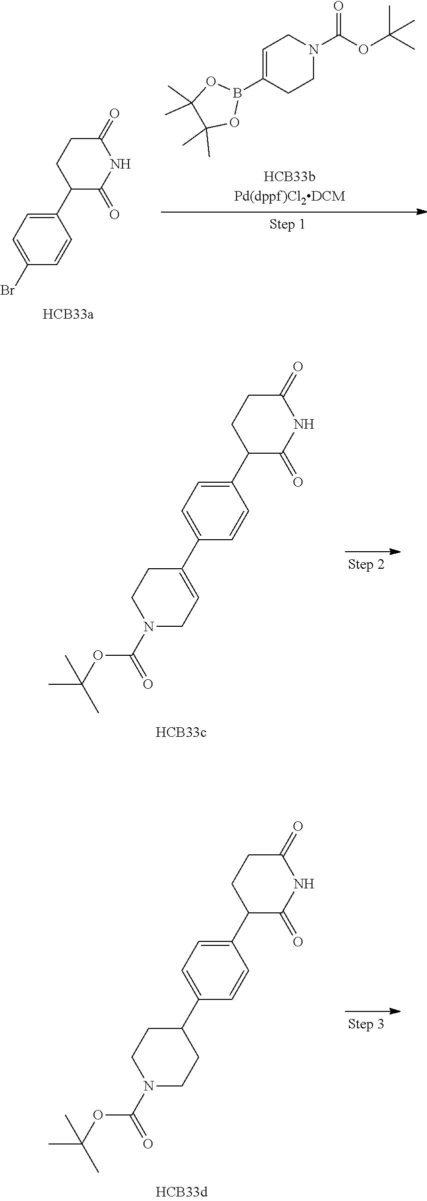

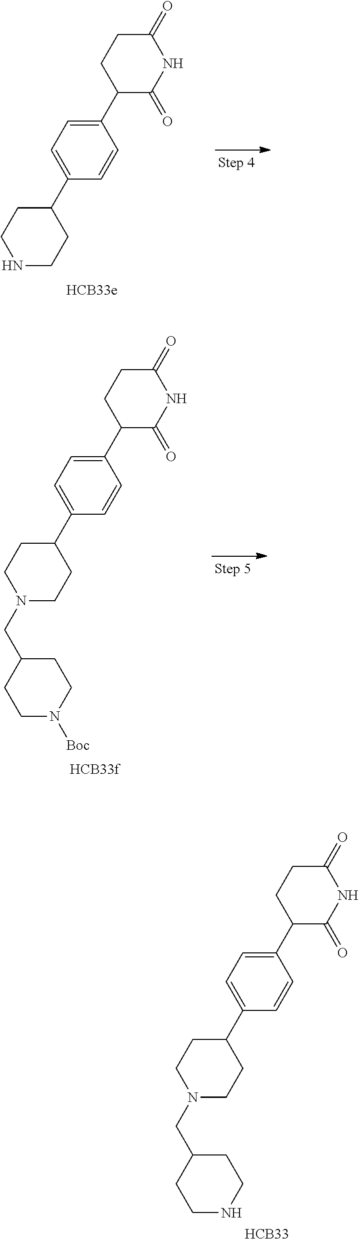 [SMILES: O=C1CCC(c2ccc(C3CCNCC3)cc2)C(=O)N1 ; CC(C)(C)OC(=O)N1CCC(CN2CCC(c3ccc(C4CCC(=O)NC4=O)cc3)CC2)CC1 ; "HCB33" ; O=C1CCC(c2ccc(C3CCN(CC4CCNCC4)CC3)cc2)C(=O)N1]

### Step 1: tert-butyl 4-[4-(2,6-dioxopiperidin-3-yl)phenyl]-1,2,3,6-tetrahydropyridine-1-carboxylate (HCB33c)

To an argon purged flask containing N-Boc-1,2,5,6-tetrahydropyridine-4-boronic acid pinacol ester (1.7 g, 5.22 mmol, 1.0 equiv), K₂CO₃ (2.2 g, 15.66 mmol, 3.0 equiv), and Pd(dppf)Cl₂.DCM (0.2 g, 0.26 mmol, 0.05 equiv) in DMF (15 mL, 0.35 M) was added 3-(4-bromophenyl)-piperidine-2,6-dione (1.4 g, 5.22 mmol, 1.0 equiv). The mixture was heated at 85° C. under argon overnight (the reaction was monitored by UPLC). The mixture was filtered from insoluble materials, and all volatiles were evaporated in vacuo. The resulting residue was purified by flash chromatography (20 to 100% EtOAc in Hexane) to give the title product as a white solid (1.35 g, 66%). ESI (−) [M−H]⁻=369.10. ¹H NMR (300 MHz, Chloroform-d), δ 7.98 (s, 1H), 7.40 (d, J=8.4 Hz, 2H), 7.20 (d, J=8.4 Hz, 2H), 6.06 (m, 1H), 4.11 (m, 2H), 3.78 (m, 1H), 3.65 (t, J=5.7 Hz, 2H), 2.75 (m, 2H), 2.53 (s, 2H), 2.34 (s, 2H), 1.51 (s, 9H).

### Step 2: tert-Butyl 4-[4-(2,6-dioxopiperidin-3-yl)phenyl]piperidine-1-carboxylate (HCB33d)

A suspension of benzyl 4-{4-[(tert-butoxy)carbonyl]phenyl}-1,2,3,6-tetrahydropyridine-1-carboxylate (1.411 g, 3.618 mmol, 1.0 equiv) and 5% Pd(OH)₂ (0.07 equiv) in THF (80.38 mL, 0.045 M) was vigorously stirred under H₂ atmosphere (balloon) for 3 h. Reduction progress was monitored by UPLC and NMR. The solids were filtered, washed with THF and the solution was evaporated to give a white crystalline product (1.26 g, 89%). LCMS: 254 nm, Rₜ=3.73 min, 96.21%, ESI (−) [M−H]⁻=371.59. ¹H NMR (300 MHz, Chloroform-d), δ 8.04 (s, 1H), 7.22 (d, J=8.2 Hz, 2H), 7.16 (d, J=8.2 Hz, 2H), 4.26 (d, J=13.3 Hz, 2H), 3.78 (m, 1H), 2.88-2.61 (m, 5H), 2.31 (m, 2H), 1.65 (m, 2H), 1.65 (m, 2H), 1.50 (s, 9H).

Step 3: tert-butyl 4-{4-[(3R)-2,6-dioxopiperidin-3-yl]phenyl}piperidine-1-carboxylate (40.00 mg, 0.11 mmol) was dissolved in 4 N HCl in dioxane (3 mL) and stirred for 30 minutes. The volatiles were removed, and the desired product was placed under vacuum to dryness. The crude material was used in the next step without purification. LCMS: C₁₆H₂₀N₂O₂ requires: 272, found: m/z=273 [M+H]⁺.

Step 4: To a solution of (3R)-3-[4-(piperidin-4-yl)phenyl]piperidine-2,6-dione (38.00 mg, 0.14 mmol) and t-butyl 4-formylpiperidine-1-carboxylate (30 mg, 0.14 mmol) in DMSO (1 mL), TEA (20 mL, 0.14 mmol) and sodium triacetoxyborohydride (89 mg, 0.42 mmol) were added and the reaction was stirred for 30 mins. LCMS indicated the completion of reaction. The reaction was quenched with water and extracted with EtOAc. The organic layer was washed with saturated aqueous NaCl, dried over Na₂SO₄, and concentrated under reduced pressure. Silica gel chromatography purification provided the desired product (35 mg, 0.07 mmol, 53%). LCMS: C₂₇H₃₉N₃O₄ requires: 469, found: m/z=470 [M+H]⁺.

Step 5: tert-butyl 4-[(4-{4-[(3R)-2,6-dioxopiperidin-3-yl]phenyl}piperidin-1-yl)methyl]piperidine-1-carboxylate as a colorless oil (35 mg, 0.16 mmol) was dissolved in DCM (1 mL) and 4N HCl in dioxane (2 mL) was added, The reaction was stirred for one hour. All the solvents were removed under reduced pressure to afford 3-{4-[1-(piperidin-4-ylmethyl)piperidin-4-yl]phenyl}piperidine-2,6-dione hydrogen chloride salt (quantitative yield). LCMS: C₂₄H₃₁N₃O₂ requires: 369, found: m/z=370 [M+H]⁺.

### Example 13. 3-(4-bromophenyl)piperidine-2,6-dione synthesis (HCB33a)

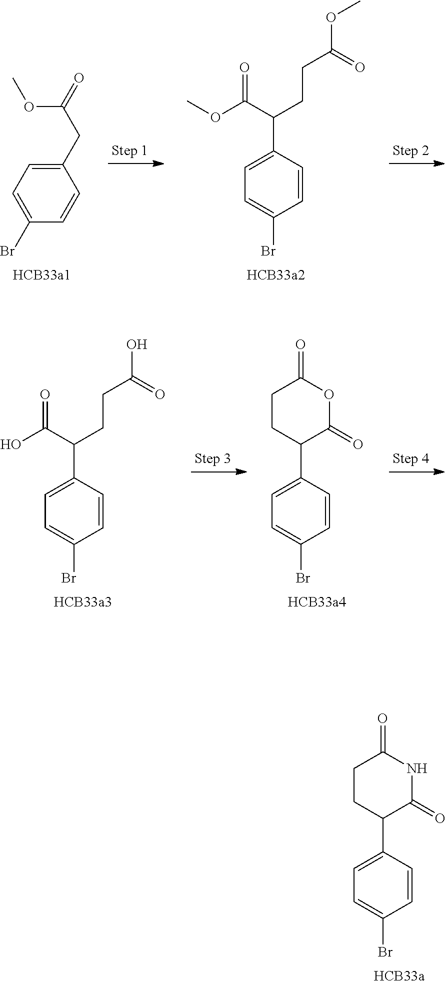

### Step 1: 1,5-dimethyl 2-(4-bromophenyl)pentanedioate (HCB33a2)

A solution of methyl 4-bromophenylacetate (10.2 g, 43.65 mmol, 1.0 equiv) in anhydrous toluene (30 mL) was added dropwise to a suspension of potassium tert-butoxide (1.01 g, 8.731 mmol, 0.2 equiv) in toluene (50.0 mL) under nitrogen atmosphere at −70° C. The resulting mixture was stirred for fifteen minutes and a solution of 18-crown-6-ether (1.1 g, 4.36 mmol, 0.1 equiv) in toluene (10 mL) was added. Then, a solution of methyl acrylate (3.8 g, 43.65 mmol, 1 equiv) in toluene (20 mL) was added and the slurry reaction mixture was stirred for one hour at low temperature and two hours at ambient temperature. Afterward, the reaction was poured into a saturated aqueous HCl solution (100 mL), and the product was extracted with diethyl ether, dried over Na₂SO₄, concentrated in vacuo, and purified by flash chromatography (0 to 15% EtOAc in Hexane) to give the desired compound as a colorless oil (9.2 g, 64%). ¹H NMR (300 MHz, Chloroform-d), δ 7.45 (d, J=8.4 Hz, 2H), 7.16 (d, J=8.4 Hz, 2H), 3.74-3.50 (m, 7H), 2.37 (m, 1H), 2.26 (m, 2H), 2.10 (m, 1H).

### Step 2: 2-(4-bromophenyl)pentanedioic acid (HCB33a3)

A solution of 1,5-dimethyl-2-(4-bromophenyl)pentanedioate (4.7 g, 14.27 mmol, 1 equiv) and potassium hydroxide (2.1 g, 35.69 mmol, 2.5 equiv) in THF:Water (1:1) (30 mL, 0.4 M) was stirred at room temperature for 2 h, and then one hour at 90° C. The reaction progress was monitored by NMR or UPLC. Upon completion, the resulting solution was neutralized with dilute HCl to pH=5, extracted with diethyl ether, and dried over Na₂SO₄. All volatiles were evaporated at reduced pressure to give an off-white solid product (3.95 g, 92%). ESI (−) [M−H]⁻=286.75. ¹H NMR (300 MHz, Chloroform-d), δ 7.49 (d, J=8.4 Hz, 2H), 7.21 (d, J=8.4 Hz, 2H), 3.65 (m, 1H), 2.50-2.36 (m, 3H), 2.10 (m, 1H).

### Step 3: 3-(4-bromophenyl)oxane-2,6-dione (HCB33a4)

A solution of 2-(4-bromophenyl)-pentanedioic acid (0.526 g, 1.74 mmol, 1 equiv) in acetic anhydride (3.28 ml, 34.82 mmol, 20 equiv) was heated at reflux for 1.5 h. All volatiles were removed by distillation at low pressure and the crude residue was co-evaporated twice with toluene to give the desired compound (0.43 g, 83%). ¹H NMR (300 MHz, Chloroform-d), δ 7.55 (d, J=8.4 Hz, 2H), 7.14 (d, J=8.4 Hz, 2H), 3.88 (m, 1H), 3.09-2.77 (m, 2H), 2.30 (m, 2H).

### Step 4: 3-(4-bromophenyl)piperidine-2,6-dione (HCB33a)

A mixture of the 3-(4-bromophenyl)oxane-2,6-dione (3.0 g, 10.0 mmol, 1.0 equiv) and well-ground ammonium carbonate (4.9 g, 50.17 mmol, 5.0 equiv) was heated to 190-200° C. for 120 minutes in a 100 mL flask equipped with an air condenser. The reaction mixture became a clear liquid that solidified upon cooling. The residue was triturated with warm 50% aqueous ethanol which converted to a slurry of crystals. The crystals were collected, washed with 50% aqueous ethanol, and dried to give a gray crystalline solid (2.0 g, 71%). LCMS: 254 nm, Rₜ=2.42 min, 98.68%, ESI (−) [M−H]⁻=267.70. ¹H NMR (300 MHz, DMSO-d₆), δ 10.86 (s, 1H), 7.53 (d, J=8.4 Hz, 2H), 7.20 (d, J=8.4 Hz, 2H), 3.88 (m, 1H), 2.62 (m, 1H), 2.54 (s, 1H), 2.17 (m, 1H), 2.01 (m, 1H).

### Example 14. 3-(4-(4-(piperazin-1-yl)but-1-yn-1-yl)phenyl)piperidine-2,6-dione (HCB34)

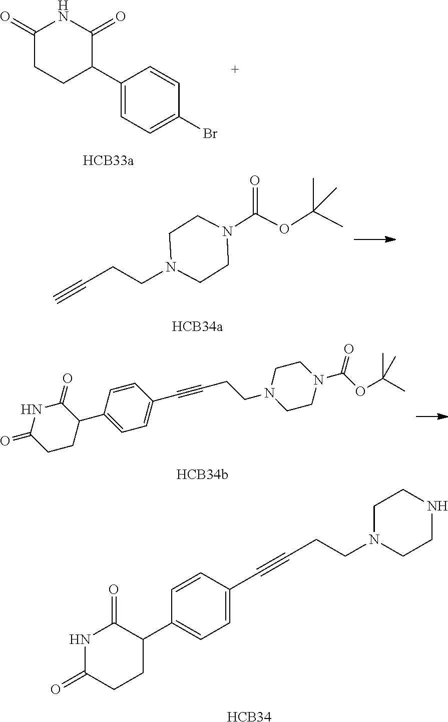

Step 1: To a solution of 3-(4-bromophenyl)piperidine-2,6-dione (50.00 mg, 0.19 mmol) and tert-butyl 4-(but-3-yn-1-yl)piperazine-1-carboxylate (89 mg, 0.37 mmol) in THF (2 mL) was added palladium bis(triphenylphosphine) chloride.dichloromethane (13.09 mg, 0.02 mmol), copper(I) iodide (7.10 mg, 0.04 mmol), and triethylamine (0.36 mL, 0.26 g, 2.61 mmol). Then, nitrogen gas was bubbled through the mixture for five minutes. The reaction mixture was heated at 60° C. for one hour. Additional equivalent of tert-butyl 4-(but-3-yn-1-yl)piperazine-1-carboxylate (45 mg, 0.19 mmol) was added, and the reaction was heated at 60° C. for 16 hours. The reaction mixture was diluted with DCM (25 mL) and washed sequentially with water (3 mL), 10% citric acid in water (3 mL), and saturated brine (3 mL). The organic layer was dried with Na₂SO₄, filtered, and evaporated under reduced pressure to afford crude product. The crude product was purified by silica gel column chromatography (ISCO elution gradient 0 to 5% MeOH in DCM) to afford tert-butyl 4-{4-[4-(2,6-dioxopiperidin-3-yl)phenyl]but-3-yn-1-yl}piperazine-1-carboxylate (36 mg, 45%). LCMS: C₂₄H₃₁N₃O₄ requires: 425.2, found: m/z=426.5 [M+H]⁺.

Step 2: tert-butyl 4-{4-[4-(2,6-dioxopiperidin-3-yl)phenyl]but-3-yn-1-yl}piperazine-1-carboxylate (36 mg, 0.08 mmol) was dissolved in 5% trifluoroacetic acid in hexafluoro isopropanol solution (5 mL), and left to stand for 30 minutes. Trifluoroacetic acid and hexafluoroisopropanol were removed. The residue was placed under vacuum to dryness to provide 3-{4-[4-(piperazin-1-yl)but-1-yn-1-yl]phenyl}piperidine-2,6-dione (quantitative yield). LCMS: C₁₉H₂₃N₃O₂ requires: 325.2, found: m/z=326.5 [M+H]⁺.

Specific examples of preparing ITK hook building blocks (BB X₁ to X₂₁) are described in detail below.

### Example 15. (4aS,5aR)-5,5-difluoro-5a-methyl-1,4,4a,5,5a,6-hexahydrocyclopropa[f]indazole-3-carboxylic acid (BBX₁)

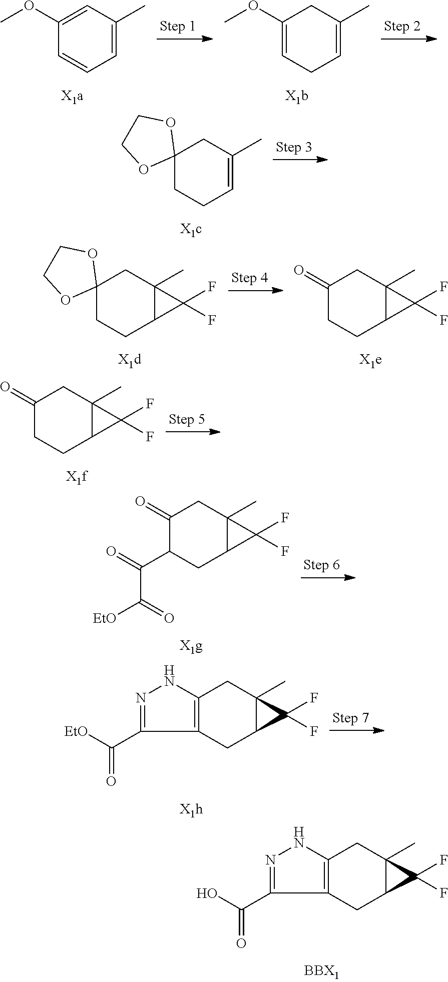

### Step 1: 1-Methoxy-5-methylcyclohexa-1,4-diene (X₁a)

A solution of 3-methyl anisole (180 g, 122.78 mol, 1.0 equiv) in diethyl ether (982.24 mL, 1.5 M) and tert-butyl alcohol (982 mL, 1.5 M) was added dropwise to liquid ammonia (2947 mL, 0.5 M) at −78° C., then sodium (152 g, 6.63 mol, 3.0 equiv) was added in portions. The resulting solution was warmed to −35° C., stirred at −35° C. for two h, and then at rt overnight. The resulting suspension was diluted with pentane, quenched with Na₂SO₄.10H₂O, then water (carefully and very slowly), extracted with pentane, dried over anhydrous sodium sulfate, and concentrated in vacuum. This resulted in 1-methoxy-5-methylcyclohexa-1,4-diene as a colorless oil (170 g, 92.9% yield). ¹H NMR (300 MHz, Chloroform-d) δ 5.43 (tt, J=3.5, 1.7 Hz, 1H), 4.66 (d, J=3.8 Hz, 1H), 3.58 (s, 3H), 2.87-2.73 (m, 2H), 2.63 (t, J=7.7 Hz, 2H), 1.72 (d, J=2.1 Hz, 3H).

### Step 2: 7-Methyl-1,4-dioxaspiro[4.5]dec-7-ene (X₁c)

Formic acid (47.5 mL, 1.26 mmol, 1.03 equiv) was added to a solution 1-methoxy-5-methylcyclohexa-1,4-diene (151.77 g, 124.18 mmol, 1.0 equiv) in ethylene glycol (1530 mL, 0.8 M) at room temperature. After being stirred for 16 h, the reaction mixture was quenched by saturated aqueous NaHCO₃. The resulting mixture was extracted with pentane. The combined organic extracts were washed with H₂O, brine, and dried over Na₂SO₄. The resulting solution was concentrated to give 155.2 g of 7-methyl-1,4-dioxaspiro[4.5]dec-7-ene as a colorless oil (yield 80.675%). ¹H NMR (300 MHz, Chloroform-d) δ 5.44 (tq, J=3.5, 1.8 Hz, 1H), 4.06-3.92 (m, 4H), 2.29-2.16 (m, 4H), 1.78-1.66 (m, 5H).

### Step 3: 7,7-Difluoro-1-methylspiro[bicyclo[4.1.0]heptane-3,2-[1,3]dioxolane] (X₁d)

NaI (4.218 g, 28.14 mmol, 0.5 equiv), THF (176 mL, 0.32 M), 7-methyl-1,4-dioxaspiro[4.5]dec-7-ene (8.68 g, 56.29 mmol, 1.0 equiv), and TMSCF₃ (20.78 mL, 140.72 mmol, 2.5 equiv) were weighed into a 500 mL medium pressure reactor. The tube was purged with argon and stirred at 65° C. for 12 h and then quenched with 20 mL of water. The resulting solution was extracted with ethyl acetate, washed with saturated Na₂CO₃, brine, dried over anhydrous sodium sulfate, and concentrated under vacuum to give 11.49 g (quant) of 7,7-difluoro-1-methylspiro[bicyclo[4.1.0]heptane-3,2-[1,3]dioxolane] as a colorless oil. ¹H NMR (300 MHz, Chloroform-d) δ 4.05-3.87 (m, 4H), 2.18-1.84 (m, 3H), 1.69 (ddd, J=14.5, 4.3, 2.1 Hz, 1H), 1.59-1.36 (m, 2H), 1.27 (dd, J=2.7, 1.8 Hz, 3H), 1.17 (dddd, J=14.8, 6.7, 3.8, 1.8 Hz, 1H).

### Step 4: 7,7-Difluoro-1-methylbicyclo[4.1.0]heptan-3-one (X₁e)

A solution of 7,7-difluoro-1-methylspiro[bicyclo[4.1.0]heptane-3,2-[1,3]dioxolane] (11.50 g, 56.29 mmol, 1.0 equiv), TsOH.H₂O (1.071 g, 190.22 mmol, 0.1 equiv) in acetone (188 mL, 0.3 M) and water (37.5 mL, 1.5 M) were stirred for 48 h at 50° C. The reaction mixture was diluted with diethyl ether. The organic layer was washed with saturated sodium bicarbonate solution, brine, dried over anhydrous sodium sulfate, and concentrated under vacuum to give 8.52 g of 7,7-difluoro-1-methylbicyclo[4.1.0]heptan-3-one as a light-yellow oil (yield 94.5%). ¹H NMR (300 MHz, Chloroform-d) δ 2.59 (dd, J=17.5, 2.3 Hz, 1H), 2.41-2.13 (m, 4H), 2.08-1.85 (m, 1H), 1.55-1.37 (m, 1H), 1.31 (dd, J=2.9, 1.8 Hz, 3H).

### Step 5: Ethyl 2-(7,7-difluoro-6-methyl-4-oxobicyclo[4.1.0]heptan-3-yl)-2-oxoacetate (X₁g)

A solution of 7,7-difluoro-1-methylbicyclo[4.1.0]heptan-3-one (1.0 g, 6.24 mmol, 1.0 equiv) in 2.5 mL Et₂O was added to a solution of LHMDS 1 M in THF (6.24 mL, 6.24 mmol, 1.0 equiv) in 10 mL of Et₂O at −78° C. under argon. The mixture was maintained at −78° C. and stirred for 60 min. A solution of diethyl oxalate (0.922 g, 6.87 mmol, 1.01 equiv) in 2.5 mL Et₂O was added to the mixture to adjust the total amount of Et₂O (15 mL, 0.2 M). The reaction was stirred for one hour at −78° C. The reaction mixture was allowed to slowly warm to rt over 16 h. The reaction was quenched with 1N HCl. The organic layer was extracted with Et₂O, washed with brine and separated, then dried with anhydrous sodium sulfate, filtered and concentrated in vacuo to yield 1.375 g of oxo-(2-oxo-cyclohexyl)-acetic acid ethyl ester as a yellow oil (yield 84.6%). ¹H NMR (300 MHz, Chloroform-d) δ 15.04 (s, 1H), 4.38 (q, J=7.1 Hz, 2H), 3.18 (ddd, J=16.9, 7.0, 2.7 Hz, 1H), 2.85-2.66 (m, 2H), 2.56 (dd, J=18.1, 2.9 Hz, 1H), 1.56-1.45 (m, 1H), 1.41 (t, J=7.1 Hz, 4H), 1.32 (dd, J=2.8, 1.7 Hz, 3H).

### Step 6: ethyl (4aS,5aR)-5,5-difluoro-5a-methyl-1,4,4a,5,5a,6-hexahydrocyclopropa[f]indazole-3-carboxylate (X₁h)

To a solution of ethyl 2-[7,7-difluoro-6-methyl-4-oxobicyclo[4.1.0]heptan-3-yl]-2-oxoacetate (55.2 g, 212.11 mmol) in acetic acid (500 mL) was added hydrazine hydrate (67.9 g, 1.36 mol) dropwise at room temperature. The resulting mixture was stirred at 120° C. for 16 h before concentration under vacuum. The residue was diluted with water and extracted with ethyl acetate. The combined organic layers were washed with water and brine, dried over anhydrous sodium sulfate and concentrated under vacuum. The crude residue was purified by flash column chromatography with 0˜50% ethyl acetate in petroleum ether to afford ethyl 5,5-difluoro-5a-methyl-1H,4H,4aH,5H,5aH,6H-cyclopropa[f]indazole-3-carboxylate (23 g, 42%) as a yellow solid. The racemic product (10 g) was separated by prep-chiral SFC under the following conditions: [Column, AD-H; Column temperature 35° C.; Co-Solvent MeOH (0.2% DIEA) 50.56%; Co-Solvent flow rate 90 mL/min; Total flow 178 mL/min; Back pressure 100 bar; Pressure drop 90; Detector, UV 220 nm] to afford ethyl (4aR,5aS)-5,5-difluoro-5a-methyl-1,4,4a,5,5a,6-hexahydrocyclopropa[f]indazole-3-carboxylate (4.5 g) with shorter retention time on chiral-SFC as a yellow solid, and ethyl (4aS,5aR)-5,5-difluoro-5a-methyl-1,4,4a,5,5a,6-hexahydrocyclopropa[f]indazole-3-carboxylate (4.2 g) with longer retention time on chiral SFC as a yellow solid. ¹H NMR (400 MHz, Chloroform-d) δ 9.99 (s, 1H), 4.39 (q, J=7.2 Hz, 2H), 3.34-3.01 (m, 3H), 2.81 (dd, J=16.8, 3.6 Hz, 1H), 1.72-1.50 (m, 1H), 1.46-1.26 (m, 6H). ¹⁹F NMR (400 MHz, Chloroform-d) δ −136.26, −137.68, −146.15, −146.57. LCMS: C₁₂H₁₄F₂N₂O₂ requires: 256, found: m/z=257 [M+H]⁺.

### Step 7: (4aS,5aR)-5,5-Difluoro-5a-methyl-1H,4H,4aH,5H,5aH,6H-cyclopropa[f]indazole-3-carboxylic acid (BBX₁)

To a solution of ethyl (4aS,5aR)-5,5-difluoro-5a-methyl-1H,4H,4aH,5H,5aH,6H-cyclopropa[f]indazole-3-carboxylate (12.2 g, 47.609 mmol, 1.0 equiv) in ethanol (305.0 mL, 0.15 M) was added a solution of NaOH (19.522 g, 476.088 mmol, 10.0 equiv) in water (122.0 mL, 0.39 M). The resulting mixture was stirred for two hours at 50° C. The reaction mixture was concentrated under vacuum and the residue was dissolved in 200 mL of water. The pH of the solution was adjusted to 4-5 with 1 N aq. HCl solution. The solid was collected by filtration and dried under vacuum to provide the title compound as a white solid (1.805 g, 73% yield). ¹H NMR (300 MHz, DMSO-d₆) δ 12.98 (s, 2H), 3.07-2.94 (m, 3H), 2.77 (dd, J=17.3, 3.9 Hz, 1H), 1.83-1.68 (m, 1H), 1.34 (t, J=2.0 Hz, 3H). LCMS: C₁₀H₁₀F₂N₂O₂ requires: 228, found: m/z=229 [M+H]⁺.

### Example 16. tert-butyl (S)-3-((4-amino-1H-pyrazol-1-yl)(phenyl)methyl)azetidine-1-carboxylate (BBX₂)

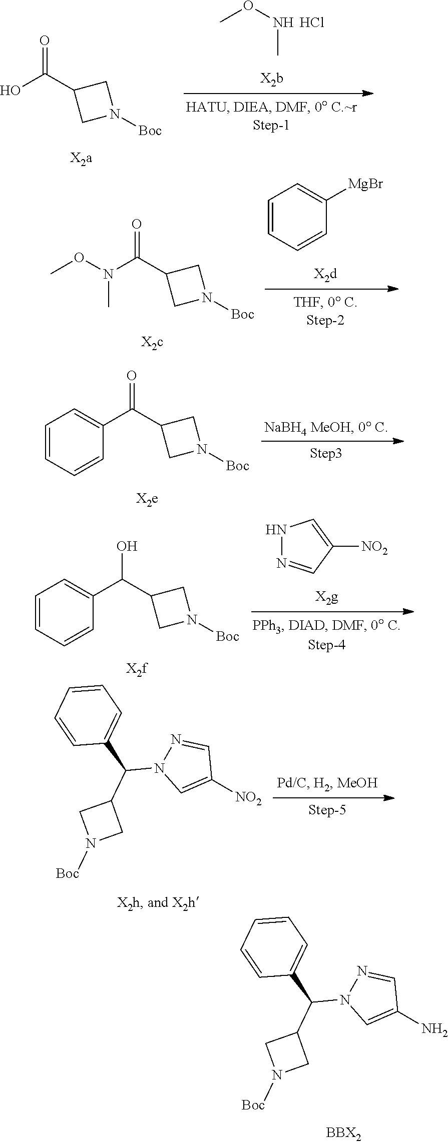

### Step 1: tert-butyl 3-[methoxy(methyl)carbamoyl]azetidine-1-carboxylate (X_{2C})

To a mixture of 1-[(tert-butoxy)carbonyl]azetidine-3-carboxylic acid (50 g, 248.48 mmol) and DIEA (299.7 g, 2.32 mol) in N,N-dimethylformamide (500 mL) were added methoxy(methyl)amine hydrochloride (48.0 g, 494.41 mmol) and HATU (115.3 g, 303.24 mmol). The resulting mixture was stirred at room temperature for 16 h. The reaction mixture was quenched with water and extracted with ethyl acetate. The combined organic layers were washed with water, brine, dried over anhydrous sodium sulfate, and concentrated under vacuum to afford tert-butyl 3-[methoxy(methyl)carbamoyl]azetidine-1-carboxylate (200 g, crude) as light yellow oil, which was used in the next step without further purification. LCMS: C₁₁H₂₀N₂O₄ requires: 244, found: m/z=245 [M+H]⁺.

### Step 2: tert-butyl 3-benzoylazetidine-1-carboxylate (X₂ₑ)

To a solution of tert-butyl 3-[methoxy(methyl)carbamoyl]azetidine-1-carboxylate (200 g, 818 mmol) in tetrahydrofuran (2 L) was added phenylmagnesium bromide (614 mL, 2 M in THF, 1.23 mol) dropwise at 0° C. under nitrogen. The resulting solution was stirred at room temperature for one hour, and then quenched with saturated NH₄Cl solution at 0-5° C. The solids were filtered out and the aqueous solution was extracted with ethyl acetate. The organic layer was washed with brine, dried over anhydrous sodium sulfate, and concentrated under vacuum. The crude residue was purified by flash column chromatography with 0-10% ethyl acetate in petroleum ether to afford tert-butyl 3-benzoylazetidine-1-carboxylate (59 g, 48% over two steps) as a white solid. LCMS: C₁₅H₁₉NO₃ requires: 261, found: m/z=262 [M+H]⁺.

### Step 3: tert-butyl 3-benzoylazetidine-1-carboxylate (X₁c)

To a solution of tert-butyl 3-benzoylazetidine-1-carboxylate (54.8 g, 209.70 mmol) in methanol (540 mL) was added NaBH₄ (16.0 g, 419.4 mmol) in portions at 0-5° C. The resulting mixture was stirred at 0-5° C. for 2 h. The reaction mixture was quenched by the addition of water maintaining at 0-5° C., and then extracted with ethyl acetate. The organic was washed with brine, dried over anhydrous sodium sulfate, and concentrated under vacuum to afford tert-butyl 3-[hydroxy(phenyl)methyl]azetidine-1-carboxylate (53 g, 96%) as yellow oil. ¹H NMR (300 MHz, DMSO-d₆) δ 7.45-7.09 (m, 5H), 5.55 (d, J=4.8 Hz, 1H), 4.64 (dd, J=7.2, 4.5 Hz, 1H), 3.80-2.65 (m, 4H), 2.76-2.74 (m, 1H), 1.38 (s, 9H). LCMS: C₁₅H₂₁NO₃ requires: 263, found: m/z=264 [M+H]⁺.

### Step 4: tert-butyl (S)-3-((4-nitro-1H-pyrazol-1-yl)(phenyl)methyl)azetidine-1-carboxylate (11A) and tert-butyl (R)-3-((4-nitro-1H-pyrazol-1-yl)(phenyl)methyl)azetidine-1-carboxylate (X₂h, and X₂h′)

To a mixture of tert-butyl 3-[hydroxy(phenyl)methyl]azetidine-1-carboxylate (50 g, 205 mmol), 4-nitro-1H-pyrazole (30 g, 260 mmol), and PPh₃ (80.5 g, 307 mmol) in THF (500 mL) was added DIAD (62 g, 307 mmol) dropwise at 0° C. under nitrogen. The resulting mixture was stirred at room temperature for 16 h under nitrogen. The reaction mixture was quenched by water and extracted with ethyl acetate. The combined organic layers were washed with water and brine, dried over anhydrous sodium sulfate, and concentrated under vacuum. The crude residue was purified by flash column chromatography with 0-10% ethyl acetate in petroleum ether to afford tert-butyl 3-[(4-nitro-1H-pyrazol-1-yl)(phenyl)methyl]azetidine-1-carboxylate (53 g, 72%) as yellow oil. The racemic product (10 g) was separated by prep-chiral-SFC under the following conditions: [Column, (R,R)WHELK-01; Column temperature 35° C.; Co-Solvent IPA (0.1% DIEA) 50.56%; Co-Solvent flow rate 90 mL/min; Total flow 178; Back pressure 1500; Detector, UV 220 nm] to afford tert-butyl (S)-3-((4-nitro-1H-pyrazol-1-yl)(phenyl)methyl)azetidine-1-carboxylate (4.5 g) as a yellow syrup with the shorter retention time on chiral-SFC and tert-butyl (R)-3-((4-nitro-1H-pyrazol-1-yl)(phenyl)methyl)azetidine-1-carboxylate (4.3 g) as a yellow syrup with the longer retention time on chiral-SFC. ¹H NMR (300 MHz, Chloroform-d) δ 8.11-7.99 (m, 2H), 7.48-7.37 (m, 3H), 7.34-7.30 (m, 2H), 5.41 (d, J=10.5 Hz, 1H), 4.17-4.06 (m, 1H), 3.95-3.94 (m, 1H), 3.80 (d, J=4.8 Hz, 1H), 3.67-3.47 (m, 2H), 1.42 (s, 9H). LCMS: C₁₈H₂₂N₄O₄ requires: 358, found: m/z=359 [M+H]⁺.

### Step 5: (S)-tert-butyl 3-((4-amino-1H-pyrazol-1-yl)(phenyl)methyl)azetidine-1-carboxylate (BBX₂)

To a solution of 3-[(S)-(4-nitro-1H-pyrazol-1-yl)(phenyl)methyl]azetidine-1-carboxylate (4.5 g, 13.67 mmol) in methanol (50 mL) was added palladium on carbon (dry, 0.5 g) under nitrogen. The resulting mixture was stirred at room temperature for 2 h under H₂ (2 atm). The solids were filtered out. The filtrate was concentrated under vacuum to afford tert-butyl 3-[(S)-(4-amino-1H-pyrazol-1-yl)(phenyl)methyl]azetidine-1-carboxylate (4.0 g, crude) as red syrup. LCMS: C₁₈H₂₄N₄O₂ requires: 328, found: m/z=329 [M+H]⁺.

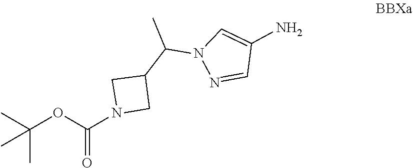 [SMILES: CC(C1CN(C(=O)OC(C)(C)C)C1)n1cc(N)cn1]

The compound BBXₐ was synthesized via the BBX₂ procedure starting from tert-butyl 3-(1-hydroxyethyl)azetidine-1-carboxylate to afford the desired product. LCMS: C₁₃H₂₂N₄O₂ requires: 266, found: m/z=267 [M+H]⁺.

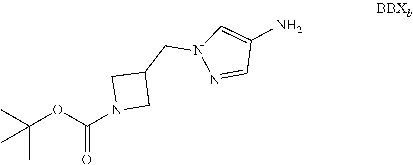 [SMILES: CC(C)(C)OC(=O)N1CC(Cn2cc(N)cn2)C1]

The compound BBX_{b} was synthesized via the BBX₂ procedure starting from tert-butyl 3-(hydroxymethyl)azetidine-1-carboxylate to afford the desired product. LCMS: C₁₂H₂₀N₄O₂ requires: 252, found: m/z=253 [M+H]⁺.

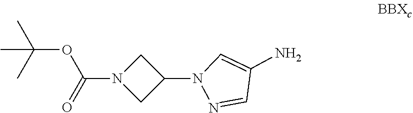 [SMILES: CC(C)(C)OC(=O)N1CC(n2cc(N)cn2)C1]

The compound BBX_{c} was synthesized via the BBX₂ procedure starting from tert-butyl 3-(4-nitro-1H-pyrazol-1-yl)azetidine-1-carboxylate to afford the desired product. LCMS: C₁₁H₁₈N₄O₂ requires: 238, found: m/z=239 [M+H]⁺.

### Example 17. (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₃)

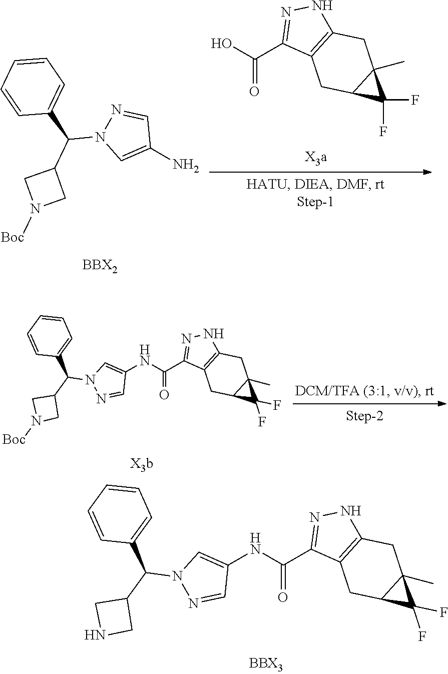 [SMILES: CC(C)(C)OC(=O)N1CC([C@@H](c2ccccc2)n2cc(N)cn2)C1 ; C[C@@]12Cc3[nH]nc(C(=O)O)c3C[C@@H]1C2(F)F ; CC(C)(C)OC(=O)N1CC([C@@H](c2ccccc2)n2cc(NC(=O)c3n[nH]c4c3C[C@@H]3C(F)(F)[C@]3(C)C4)cn2)C1 ; C[C@@]12Cc3[nH]nc(C(=O)Nc4cnn([C@H](c5ccccc5)C5CNC5)c4)c3C[C@@H]1C2(F)F]

### Step 1: tert-butyl 3-((S)-(4-((4aS,5aR)-5,5-difluoro-5a-methyl-1,4,4a,5,5a,6-hexahydrocyclopropa[f]indazole-3-carboxamido)-1H-pyrazol-1-yl)(phenyl)methyl)azetidine-1-carboxylate (X₃b)

To a stirred solution of (4aS,5aR)-5,5-difluoro-5a-methyl-1,4,4a,5,5a,6-hexahydrocyclopropa[f]indazole-3-carboxylic acid (210 mg, 0.92 mmol), tert-butyl (S)-3-((4-amino-1H-pyrazol-1-yl)(phenyl)methyl)azetidine-1-carboxylate (302 mg, 0.92 mmol), and HATU (524.4 mg, 1.38 mmol) in N,N-dimethylformamide (2 mL) was added DIEA (237.4 mg, 1.84 mmol). The resulting solution was stirred overnight at room temperature. The reaction was then quenched by the addition water and extracted with ethyl acetate. The combined organic layers were dried over anhydrous sodium sulfate and concentrated under vacuum. The residue was purified by flash column chromatography with 0-50% ethyl acetate in petroleum ether to afford tert-butyl 3-((S)-(4-((4aS,5aR)-5,5-difluoro-5a-methyl-1,4,4a,5,5a,6-hexahydrocyclopropa[f]indazole-3-carboxamido)-1H-pyrazol-1-yl)(phenyl)methyl)azetidine-1-carboxylate (340 mg, 68%) as a white solid. ¹H NMR (300 MHz, DMSO-d₆) δ 12.98 (s, 1H), 10.20 (s, 1H), 8.08 (s, 1H), 7.97 (s, 1H), 7.43-7.31 (m, 5H), 5.69 (d, J=10.4 Hz, 1H), 3.88-3.83 (m, 2H), 3.61-3.53 (m, 3H), 3.05 (s, 3H), 2.92-2.79 (m, 1H), 1.81-1.76 (m, 1H), 1.33 (s, 12H). LCMS: C₂₈H₃₂F₂N₆O₃ requires: 538, found: m/z=539 [M+H]⁺.

### Step 2: (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₃)

A solution of tert-butyl 3-((S)-(4-((4aS,5aR)-5,5-difluoro-5a-methyl-1,4,4a,5,5a,6-hexahydrocyclopropa[f]indazole-3-carboxamido)-1H-pyrazol-1-yl)(phenyl)methyl)azetidine-1-carboxylate (340 mg, 0.63 mmol) in dichloromethane (3 mL) and trifluoroacetic acid (1 mL) was stirred at room temperature for 2 h. The resulting mixture was concentrated under vacuum. The crude product was purified by reverse flash chromatography with 10˜40% MeCN in water (0.5% HCl). After concentration, the product was neutralized with 10% aq. Na₂CO₃ and extracted with propan-2-ol:CHCl₃ (1:5). The organic layer was dried over anhydrous sodium sulfate and concentrated under vacuum to afford (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (116.1 mg, 43%) as a white solid. ¹H NMR (300 MHz, DMSO-d₆) δ 12.99 (s, 1H), 10.16 (s, 1H), 8.06 (s, 1H), 7.61 (s, 1H), 7.34-7.24 (m, 5H), 5.62 (d, J=11.1 Hz, 1H), 3.69-3.58 (m, 1H), 3.47-3.22 (m, 5H), 3.05 (s, 3H), 2.84-2.77 (m, 1H), 1.81-1.73 (m, 1H), 1.34 (s, 3H). ¹⁹F NMR (282 MHz, DMSO-d₆) δ −133.52, −134.06, −145.45, −145.99. LCMS: C₂₃H₂₄F₂N₆O requires: 438, found: m/z=439 [M+H]⁺.

### Example 18. (4aS,5aR)—N-(1-(1-(azetidin-3-yl)ethyl)-1H-pyrazol-4-yl)-5,5-difluoro-5a-methyl-1,4,4a,5,5a,6-hexahydrocyclopropa[f]indazole-3-carboxamide (BBX₄)

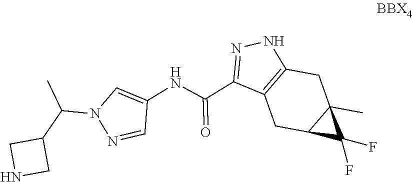 [SMILES: CC(C1CNC1)n1cc(NC(=O)c2n[nH]c3c2C[C@@H]2C(F)(F)[C@]2(C)C3)cn1]

BBX₄ was synthesized via the BBX₃ procedure starting from tert-butyl 3-(1-(4-amino-1H-pyrazol-1-yl)ethyl)azetidine-1-carboxylate. LCMS: C₁₈H₂₂F₂N₆O requires: 376, found: m/z=377 [M+H]⁺.

### Example 19. (4aS,5aR)—N-(1-(azetidin-3-ylmethyl)-1H-pyrazol-4-yl)-5,5-difluoro-5a-methyl-1,4,4a,5,5a,6-hexahydrocyclopropa[f]indazole-3-carboxamide (BBX₅)

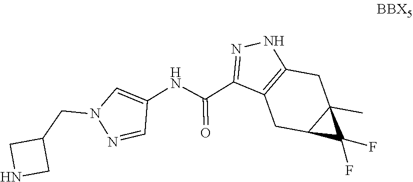 [SMILES: C[C@@]12Cc3[nH]nc(C(=O)Nc4cnn(CC5CNC5)c4)c3C[C@@H]1C2(F)F]

BBX₅ was synthesized via the BBX₃ procedure starting from tert-butyl 3-((4-amino-1H-pyrazol-1-yl)methyl)azetidine-1-carboxylate. LCMS: C₁₇H₂₀F₂N₆O requires: 362, found: m/z=363 [M+H]⁺.

### Example 20. (4aS,5aR)—N-(1-(azetidin-3-yl)-1H-pyrazol-4-yl)-5,5-difluoro-5a-methyl-1,4,4a,5,5a,6-hexahydrocyclopropa[f]indazole-3-carboxamide (BBX₆)

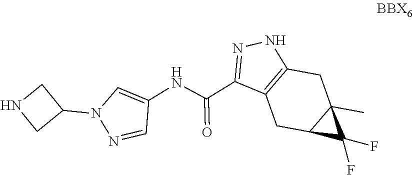 [SMILES: C[C@@]12Cc3[nH]nc(C(=O)Nc4cnn(C5CNC5)c4)c3C[C@@H]1C2(F)F]

BBX₆ was synthesized via the BBX₃ procedure starting from tert-butyl 3-(4-amino-1H-pyrazol-1-yl)azetidine-1-carboxylate. LCMS: C₁₆H₁₈F₂N₆O requires: 348, found: m/z=349 [M+H]⁺.

### Example 21. (4aS,5aR)-5,5-difluoro-N-(1-((S)-(1-(2-fluoropyridin-4-yl)azetidin-3-yl)(phenyl)methyl)-1H-pyrazol-4-yl)-5a-methyl-1,4,4a,5,5a,6-hexahydrocyclopropa[f]indazole-3-carboxamiden (BBX₇)

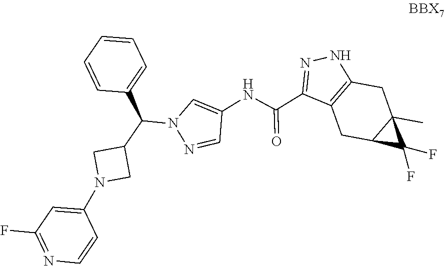 [SMILES: C[C@@]12Cc3[nH]nc(C(=O)Nc4cnn([C@H](c5ccccc5)C5CN(c6ccnc(F)c6)C5)c4)c3C[C@@H]1C2(F)F]

(4aS,5aR)—N-{1-[(S)-azetidin-3-yl(phenyl)methyl]pyrazol-4-yl}-5,5-difluoro-5a-methyl-1H,4H,4aH,6H-cyclopropa[f]indazole-3-carboxamide (291 mg, 0.57 mmol) and 2,4-difluoropyridine (53.76 μL, 68.28 mg, 0.59 mmol) were dissolved in dimethylformamide (2.69 mL, 2.53 g, 34.57 mmol) at 0° C. and N,N-diisopropylethylamine (0.1 mL, 0.57 mmol) was added dropwise while stirring. The reaction was warmed to room temperature and stirred for 20 hours. The reaction was diluted with water and extracted with EtOAc three times. The combined organic extracts were washed with brine, dried over Na₂SO₄, filtered, concentrated, and purified by flash chromatography on a 12 g column, eluting by gradient elution with 0 to 100% EtOAc:hexanes to provide (4aS,5aR)-5,5-difluoro-N-{1-[(S)-[1-(2-fluoropyridin-4-yl)azetidin-3-yl](phenyl)methyl]pyrazol-4-yl}-5a-methyl-1H,4H,4aH,6H-cyclopropa[f]indazole-3-carboxamide (0.266 g, 87.5%) as a white foam. LCMS: C₂₈H₂₆F₃N₇O requires: 533.2, found: m/z=534.5 [M+H]⁺.

### Example 22. benzyl 2-(4-{[(benzyloxy)carbonyl](methyl)amino}-2-nitrophenyl)-3-[6,6-dimethyl-1-(oxan-2-yl)-5,7-dihydro-4H-indazol-3-yl]-3-oxopropanoate (BBX₈)

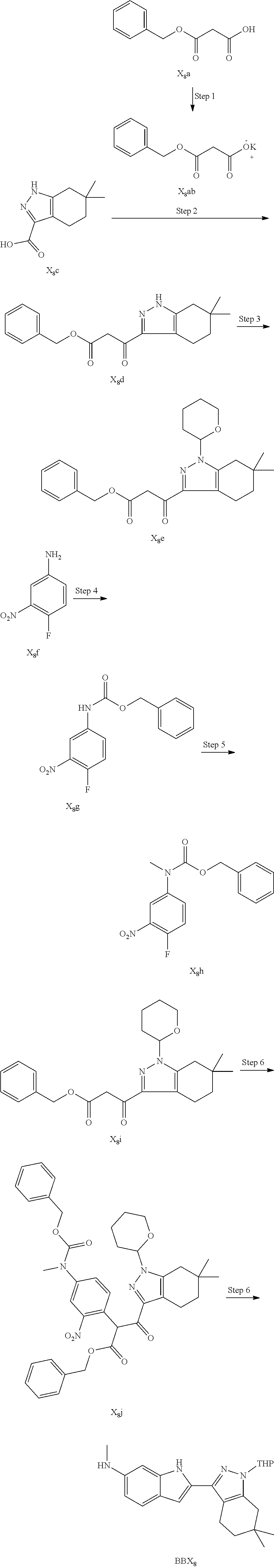

### Step 1: Potassium 3-(benzyloxy)-3-oxopropanoate (X₈b)

A solution of KOH (1.65 g) in EtOH (50 mL) was added dropwise to a solution of 3-(benzyloxy)-3-oxopropanoic acid (5.00 g, 25.75 mmol) in EtOH (25.00 mL). Ether (50 mL) was added. The resulting solid was filtered and washed with ether to provide potassium 3-(benzyloxy)-3-oxopropanoate (5.5550 g, 92.9%). ¹H NMR (500 MHz, DMSO-d₆) δ 7.43-7.27 (m, 5H), 5.03 (s, 2H), 2.92 (d, J=1.5 Hz, 2H).

### Step 2: benzyl 3-(6,6-dimethyl-1,4,5,7-tetrahydroindazol-3-yl)-3-oxopropanoate (X₈d)

6,6-dimethyl-1,4,5,7-tetrahydroindazole-3-carboxylic acid (853 mg, 4.39 mmol) and carbonyldiimidazole (783 mg, 4.83 mmol) were suspended in THF (5.00 mL). DMF (1.7 mL) was then added dropwise. In a separate flask, potassium 3-(benzyloxy)-3-oxopropanoate (2040 mg, 8.78 mmol) and magnesium chloride (1.67 g, 17.6 mmol) were suspended in THF (7 mL). DMF (7 mL) was then added dropwise. The internal temperature raised to 50° C. The resulting mixture was stirred at 60° C. for 30 minutes. The first solution, which had stirred at room temperature for one hour, was added. The resulting solution was stirred at 60° C. for three hours. The mixture was then cooled to 35° C. Toluene (10 mL) and 10% citric acid (10 mL) were then added. After stirring ten minutes, the layers were separated. The organic layer was washed with 10% citric acid (10 mL), dilute aq. NaHCO₃ (10 mL), and water (10 mL×2). The organic layer was concentrated until about 2 mL toluene remained. Heptane (˜5 mL) was added and the mixture was stirred at 60° C. for 30 minutes. Heptane (5 mL) was then added. After 30 minutes, the mixture was cooled and left to sit overnight. The mixture was concentrated in vacuo to provide benzyl 3-(6,6-dimethyl-1,4,5,7-tetrahydroindazol-3-yl)-3-oxopropanoate (1.141 g, 79.6%). LCMS: C₁₉H₂₂N₂O₃ requires: 326, found: m/z=327 [M+H]⁺.

### Step 3: benzyl 3-[6,6-dimethyl-1-(oxan-2-yl)-5,7-dihydro-4H-indazol-3-yl]-3-oxopropanoate (X₈e)

To a mixture of benzyl 3-(6,6-dimethyl-1,4,5,7-tetrahydroindazol-3-yl)-3-oxopropanoate (1.41 g, 4.32 mmol) in ethyl acetate (11.00 mL) was added para-toluene sulfonate hydrate (41.1 mg, 0.22 mmol) followed by dihydropyran (0.59 mL, 0.55 g, 6.48 mmol). After stirring overnight, the mixture was partitioned between ethyl acetate and water. The organic layer was dried over Na₂SO₄ and concentrated. The crude residue was purified by flash chromatography on a 40 g column with gradient elution 0 to 50% ethyl acetate:hexanes to provide benzyl 3-[6,6-dimethyl-1-(oxan-2-yl)-5,7-dihydro-4H-indazol-3-yl]-3-oxopropanoate (1.505 g, 84.9%). LCMS: C₂₄H₃₀N₂O₄ requires: 410, found: m/z=411 [M+H]⁺.

### Step 4: benzyl N-(4-fluoro-3-nitrophenyl)carbamate (X₈g)

To a mixture of sodium bicarbonate (1.40 g, 16.65 mmol) in water (20.00 mL) and ethyl acetate (15.00 mL) was added 4-fluoro-3-nitroaniline (2.00 g, 12.81 mmol) followed by benzyl chloroformate (2.01 mL, 2.40 g, 14.09 mmol). After stirring overnight, the layers were separated. The organic layer was washed with brine, dried over Na₂SO₄, and concentrated. The crude material was triturated from 1:10 ethyl acetate:hexanes. The resulting solid was filtered with suction to provide benzyl N-(4-fluoro-3-nitrophenyl)carbamate (3.20 g, 86.1%). ¹H NMR (500 MHz, DMSO-d₆) δ 10.29 (s, 1H), 8.35 (dd, J=6.7, 2.8 Hz, 1H), 7.77 (dt, J=9.1, 3.3 Hz, 1H), 7.56 (dd, J=11.2, 9.1 Hz, 1H), 7.50-7.33 (m, 5H), 5.20 (s, 2H).

### Step 5: Benzyl N-(4-fluoro-3-nitrophenyl)-N-methylcarbamate (X₈h)

To a mixture of benzyl N-(4-fluoro-3-nitrophenyl)carbamate (3.20 g, 11.02 mmol) in DMF (20.00 mL) was added cesium carbonate (4.31 g, 13.23 mmol) and methyl iodide (0.82 mL, 1.88 g, 13.23 mmol). After three hours, the mixture was partitioned between ethyl acetate and water. The organic layer was dried over Na₂SO₄ and concentrated in vacuo to provide benzyl N-(4-fluoro-3-nitrophenyl)-N-methylcarbamate (3.275 g, 97.7%). ¹H NMR (500 MHz, DMSO-d₆) δ 8.20 (dd, J=6.7, 2.8 Hz, 1H), 7.83 (dt, J=8.9, 3.4 Hz, 1H), 7.62 (dd, J=11.0, 9.0 Hz, 1H), 7.41-7.29 (m, 5H), 5.16 (s, 2H), 3.31 (s, 3H).

### Step 6: benzyl 2-(4-{[(benzyloxy)carbonyl](methyl)amino}-2-nitrophenyl)-3-[6,6-dimethyl-1-(oxan-2-yl)-5,7-dihydro-4H-indazol-3-yl]-3-oxopropanoate (X₈j)

To a mixture of benzyl 3-[6,6-dimethyl-1-(oxan-2-yl)-5,7-dihydro-4H-indazol-3-yl]-3-oxopropanoate (1.50 g, 3.67 mmol) in DMSO (10.00 mL) was added benzyl N-(4-fluoro-3-nitrophenyl)-N-methylcarbamate (1.12 g, 3.67 mmol) followed by potassium carbonate (1.01 g, 7.33 mmol). The mixture was heated at 90° C. for two hours. After sitting overnight at room temperature, the mixture was then heated at 90° C. for two more hours. The mixture was then cooled and partitioned between ethyl acetate and 5% aq. citric acid. The organic layer was washed with water, dried over Na₂SO₄, and concentrated to provide benzyl 2-(4-{[(benzyloxy)carbonyl](methyl)amino}-2-nitrophenyl)-3-[6,6-dimethyl-1-(oxan-2-yl)-5,7-dihydro-4H-indazol-3-yl]-3-oxopropanoate (2.3960 g, 94.1%). LCMS: C₃₉H₄₂N₄O₈ requires: 694, found: m/z=695 [M+H]⁺.

### Step 7: 2-(6,6-dimethyl-1-(tetrahydro-2H-pyran-2-yl)-4,5,6,7-tetrahydro-1H-indazol-3-yl)-N-methyl-1H-indol-6-amine (BBX₈)

Benzyl 2-(4-{[(benzyloxy)carbonyl](methyl)amino}-2-nitrophenyl)-3-[6,6-dimethyl-1-(oxan-2-yl)-5,7-dihydro-4H-indazol-3-yl]-3-oxopropanoate (112 mg, 0.16 mmol) (see US 2017/0253577 A1, the contents of which are incorporated herein their entirety) and 10% Pd/C (11 mg) in toluene (1.00 mL) and EtOH (1.00 mL) were stirred under a balloon of H₂ overnight. The mixture was diluted with THF and then filtered through celite. The mixture was concentrated until about one milliliter solvent remained. Toluene (3 mL) was then added. The mixture was then concentrated down to about one milliliter and the resulting solution of 2-(6,6-dimethyl-1-(tetrahydro-2H-pyran-2-yl)-4,5,6,7-tetrahydro-1H-indazol-3-yl)-N-methyl-1H-indol-6-amine was used in the next step without purification. LCMS: C₂₃H₃₀N₄O requires: 378, found: m/z=379 [M+H]⁺.

### Example 23. (S)—N-(2-(6,6-dimethyl-4,5,6,7-tetrahydro-1H-indazol-3-yl)-1H-indol-6-yl)-N-methyl-2-(piperazin-1-yl)propanamide (BBX₉)

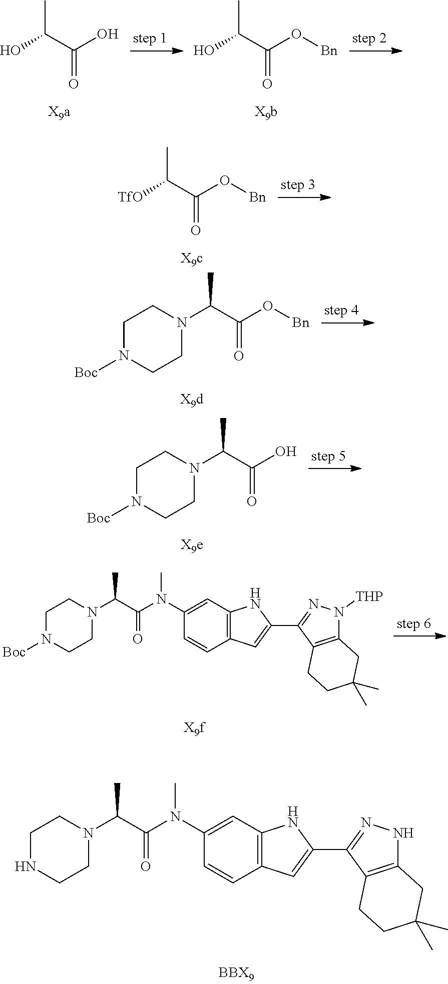

### Step 1: benzyl (R)-2-hydroxypropanoate (X₉a)

D-lactic acid (4.06 g, 45.1 mmol) was dissolved in MeOH (40 mL). 20% solution of cesium carbonate (7.34 g, 22.5 mmol) in water was added until the solution reached pH 7. The mixture was then concentrated. The resulting material was dissolved in toluene and concentrated in vacuo twice. The resulting material was then dissolved in DMF (100 mL). Benzyl bromide (6.43 mL, 54.1 mmol) was added. After stirring overnight, the mixture was then concentrated. 100 mL Ethyl acetate (100 mL) was then added, and the mixture was filtered. The resulting solution was washed with water, washed with brine, and concentrated in vacuo. The crude residue was purified by flash chromatography on an 80 g column eluted with 0 to 30% ethyl acetate:DCM and then was re-purified by flash chromatography on an 80 g column eluted with 0 to 35% ethyl acetate:hexanes to provide benzyl (2R)-2-hydroxypropanoate (4.36 g, 54%). LCMS: C₁₀H₁₂O₃ requires: 180, found: m/z=203 [M+Na]⁺.

### Step 2: benzyl (R)-2-(((trifluoromethyl)sulfonyl)oxy)propanoate (X₉c)

To a mixture of benzyl (2R)-2-hydroxypropanoate (2.79 g, 15.5 mmol) and lutidine (1.9 mL, 16.3 mmol) in DCM (75 mL) at 0° C. was added triflic anhydride (2.67 mL, 16.3 mmol). After one hour, the mixture was concentrated in vacuo. The crude material was purified by flash chromatography on a 120 g column eluted with 0 to 100% ethyl acetate:hexanes to provide benzyl (2R)-2-(trifluoromethanesulfonyloxy)propanoate (3.94 g, 81%). ¹H NMR (500 MHz, CDCl₃) δ 7.45-7.35 (m, 5H), 5.34-5.25 (m, 3H), 1.74 (d, J=7.0 Hz, 3H).

### Step 3: tert-butyl (S)-4-(1-(benzyloxy)-1-oxopropan-2-yl)piperazine-1-carboxylate (X₉d)

A mixture of benzyl (2R)-2-(trifluoromethanesulfonyloxy)propanoate (3.94 g, 12.6 mmol) in MeCN (30 mL) was added to a mixture of tert-butyl piperazine-1-carboxylate (2.35 g, 12.6 mmol) and potassium carbonate (3.49 g, 25.2 mmol) in MeCN (30 mL). The resulting mixture was heated at 60° C. for one hour. The resulting mixture was filtered. The solids were then washed with DCM. The resulting solution was concentrated onto silica gel and purified by flash chromatography on an 80 g column eluted with 0 to 50% ethyl acetate:hexanes to provide tert-butyl 4-[(2S)-1-(benzyloxy)-1-oxopropan-2-yl]piperazine-1-carboxylate (4.1 g, 93%). LCMS: C₁₉H₂₈N₂O₄ requires: 348, found: m/z=349 [M+H]⁺.

### Step 4: (S)-2-(4-(tert-butoxycarbonyl)piperazin-1-yl)propanoic acid (X₉e)

A mixture of tert-butyl 4-[(2S)-1-(benzyloxy)-1-oxopropan-2-yl]piperazine-1-carboxylate (4.1 g, 11.8 mmol) and 10% Pd/C (410 mg) was stirred in EtOH (50 mL) under a balloon of H₂ for 90 minutes. The mixture was then filtered through celite which was then washed with MeOH and DCM. The combined solution was concentrated to provide (2S)-2-[4-(tert-butoxycarbonyl)piperazin-1-yl]propanoic acid (2.88 g, 95%). LCMS: C₁₂H₂₂N₂O₄ requires: 258, found: m/z=259 [M+H]⁺.

### Step 5: tert-butyl 4-((2S)-1-((2-(6,6-dimethyl-1-(tetrahydro-2H-pyran-2-yl)-4,5,6,7-tetrahydro-1H-indazol-3-yl)-1H-indol-6-yl)(methyl)amino)-1-oxopropan-2-yl)piperazine-1-carboxylate (X₉f)

N,N-diisopropylethylamine (0.45 mL, 2.60 mmol) was added to a mixture of (2S)-2-[4-(tert-butoxycarbonyl)piperazin-1-yl]propanoic acid (168 mg, 0.65 mmol) and HATU (247 mg, 0.65 mmol) in DMF (3 mL). After five minutes, the resulting mixture was added to a solution of 2-[6,6-dimethyl-1-(oxan-2-yl)-5,7-dihydro-4H-indazol-3-yl]-N-methyl-1H-indol-6-amine (246 mg, 0.65 mmol) in toluene (1 mL). After 90 minutes, water was added and the mixture was extracted with ethyl acetate. Brine was added to get the layers to separate. The organic layer was dried over Na₂SO₄ and concentrated in vacuo. The crude residue was purified by flash chromatography on a 24 g column eluted with 0 to 5% MeOH:DCM to provide tert-butyl 4-[(1S)-1-({2-[6,6-dimethyl-1-(oxan-2-yl)-5,7-dihydro-4H-indazol-3-yl]-1H-indol-6-yl}(methyl)carbamoyl)ethyl]piperazine-1-carboxylate (0.401 g, quant.). LCMS: C₃₅H₅₀N₆O₄ requires: 618, found: m/z=619 [M+H]⁺.

### Step 6: (S)—N-(2-(6,6-dimethyl-4,5,6,7-tetrahydro-1H-indazol-3-yl)-1H-indol-6-yl)-N-methyl-2-(piperazin-1-yl)propanamide (BBX₉)

tert-butyl 4-[(1S)-1-({2-[6,6-dimethyl-1-(oxan-2-yl)-5,7-dihydro-4H-indazol-3-yl]-1H-indol-6-yl}(methyl)carbamoyl)ethyl]piperazine-1-carboxylate (401 mg, 0.65 mmol) was stirred in EtOH (2 mL) and 4 M HCl in Dioxane (2 mL, 8 mmol) for fifteen minutes. The mixture was concentrated in vacuo to provide (2S)—N-[2-(6,6-dimethyl-1,4,5,7-tetrahydroindazol-3-yl)-1H-indol-6-yl]-N-methyl-2-(piperazin-1-yl)propanamide (0.281 g, quant.) as an HCl salt. LCMS: C₂₅H₃₄N₆O requires: 434, found: m/z=435 [M+H]⁺.

### Example 24. 2-(2-(6,6-dimethyl-4,5,6,7-tetrahydro-1H-indazol-3-yl)-1H-indol-6-yl)-2,8-diazaspiro[4.5]decan-1-one (BBX₁₀)

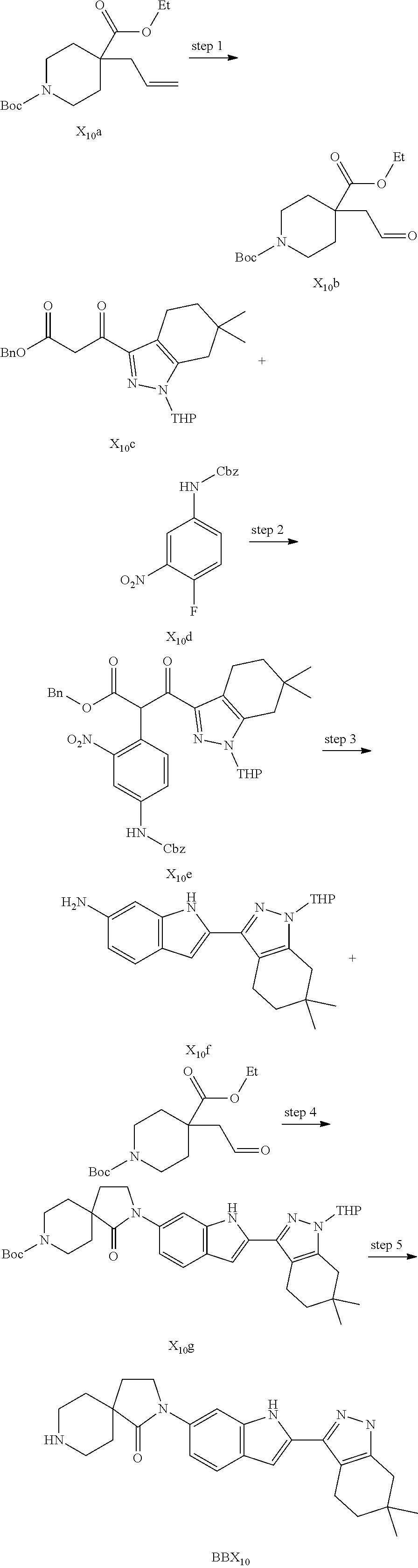

### Step 1: 1-tert-butyl 4-ethyl 4-(2-oxoethyl)piperidine-1,4-dicarboxylate (X₁₀b)

To a mixture of 1-tert-butyl 4-ethyl 4-(prop-2-en-1-yl)piperidine-1,4-dicarboxylate (2.23 g, 7.51 mmol) in THF (11.00 mL) and water (11.00 mL) was added osmium tetroxide (80.3 mg, 0.32 mmol). Five minutes later sodium periodate (4.01 g, 18.77 mmol) was added. After three hours, the mixture was partitioned between ethyl acetate and aq. NaHCO₃. The organic layer was dried over Na₂SO₄ and concentrated. The crude residue was purified by flash chromatography on a 40 g column with gradient elution 0 to 40% ethyl acetate:hexanes to provide (1.7670 g, 78.6%). LCMS: C₁₅H₂₅NO₅ requires: 299, found: m/z=322 [M+Na]⁺.

### Step 2: benzyl 2-(4-{[(benzyloxy)carbonyl]amino}-2-nitrophenyl)-3-[6,6-dimethyl-1-(oxan-2-yl)-5,7-dihydro-4H-indazol-3-yl]-3-oxopropanoate (X₁₀f)

To a mixture of benzyl 3-[6,6-dimethyl-1-(oxan-2-yl)-5,7-dihydro-4H-indazol-3-yl]-3-oxopropanoate (606 mg, 1.48 mmol) in DMSO (4.00 mL) was added benzyl N-(4-fluoro-3-nitrophenyl)carbamate (428 mg, 1.48 mmol) and potassium carbonate (408 mg, 2.95 mmol). The mixture was heated at 90° C. for two hours. The mixture was then cooled and partitioned between ethyl acetate and 5% aq. citric acid. The organic layer was washed with brine, dried over Na₂SO₄, and concentrated in vacuo. The crude residue was purified by flash chromatography on a 40 g column eluted with 0 to 40% ethyl acetate:hexanes to provide benzyl 2-(4-{[(benzyloxy)carbonyl]amino}-2-nitrophenyl)-3-[6,6-dimethyl-1-(oxan-2-yl)-5,7-dihydro-4H-indazol-3-yl]-3-oxopropanoate (0.375 g, 37.3%). LCMS: C₃₈H₄₀N₄O₈ requires: 680, found: m/z=681 [M+H]⁺.

### Step 3: 2-[6,6-dimethyl-1-(oxan-2-yl)-5,7-dihydro-4H-indazol-3-yl]-1H-indol-6-amine (X₁₀g)

To a mixture of benzyl 2-(4-{[(benzyloxy)carbonyl]amino}-2-nitrophenyl)-3-[6,6-dimethyl-1-(oxan-2-yl)-5,7-dihydro-4H-indazol-3-yl]-3-oxopropanoate (210 mg, 0.31 mmol) in EtOH (4.00 mL) and toluene (4.00 mL) was added 10% Pd/C (21 mg). The mixture was stirred under a balloon of H₂ for eighteen hours. The mixture was diluted with THF and filtered through celite. Toluene (5 mL) was then added. The mixture was concentrated down to about one milliliter. Toluene (5 mL) was then added. The mixture was again concentrated down to about one milliliter and was used in the next step without further purification as a solution of 2-[6,6-dimethyl-1-(oxan-2-yl)-5,7-dihydro-4H-indazol-3-yl]-1H-indol-6-amine in toluene. LCMS: C₂₂H₂₈N₄O requires: 364, found: m/z=365 [M+H]⁺.

### Step 4: tert-butyl 2-(2-(6,6-dimethyl-1-(tetrahydro-2H-pyran-2-yl)-4,5,6,7-tetrahydro-1H-indazol-3-yl)-1H-indol-6-yl)-1-oxo-2,8-diazaspiro[4.5]decane-8-carboxylate (X₁₀h)

A solution of 2-[6,6-dimethyl-1-(oxan-2-yl)-5,7-dihydro-4H-indazol-3-yl]-1H-indol-6-amine (113 mg, 0.31 mmol) in toluene (1 mL) was added to 1-tert-butyl 4-ethyl 4-(2-oxoethyl)piperidine-1,4-dicarboxylate (92 mg, 0.31 mmol) in THF (2 mL) followed by acetic acid (9.3 mg, 0.15 mmol) and sodium triacetoxyborohydride (131 mg, 0.62 mmol). After one hour, the mixture was diluted with toluene (3 mL) and heated at 100° C. for 2.5 hours. The mixture was then cooled, diluted with ethyl acetate, and washed with dilute aqueous sodium bicarbonate solution. The organic layer was concentrated and the crude residue was purified by flash chromatography on a 24 g column eluted with 0 to 75% ethyl acetate:DCM to provide tert-butyl 2-{2-[6,6-dimethyl-1-(oxan-2-yl)-5,7-dihydro-4H-indazol-3-yl]-1H-indol-6-yl}-1-oxo-2,8-diazaspiro[4.5]decane-8-carboxylate (0.069 g, 37%). LCMS: C₃₅H₄₇N₅O₄ requires: 601, found: m/z=602 [M+H]⁺.

### Step 5: 2-(2-(6,6-dimethyl-4,5,6,7-tetrahydro-1H-indazol-3-yl)-1H-indol-6-yl)-2,8-diazaspiro[4.5]decan-1-one (BBX₁₀)

tert-butyl 2-{2-[6,6-dimethyl-1-(oxan-2-yl)-5,7-dihydro-4H-indazol-3-yl]-1H-indol-6-yl}-1-oxo-2,8-diazaspiro[4.5]decane-8-carboxylate (69 mg, 0.11 mmol) and para-toluene sulfonate hydrate (44 mg, 0.23 mmol) were dissolved in EtOH (2 mL) and heated to 85° C. for 25 minutes. The mixture was then cooled. Dilute aqueous sodium bicarbonate was added and the mixture was extracted three times with DCM. The combined organic layers were dried over Na₂SO₄ and concentrated to provide 2-[2-(6,6-dimethyl-1,4,5,7-tetrahydroindazol-3-yl)-1H-indol-6-yl]-2,8-diazaspiro[4.5]decan-1-one (0.043 g, 90%). LCMS: C₂₅H₃₁N₅O requires: 417, found: m/z=418 [M+H]⁺.

### Example 25. tert-butyl 2-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-1-oxo-2,8-diazaspiro[4.5]decane-8-carboxylate (BBX₁₁)

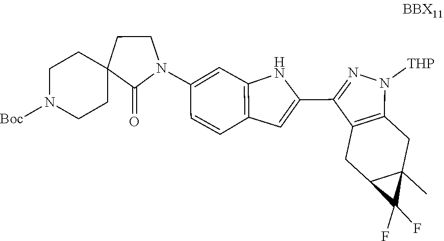

BBX₁₁ was prepared by an analogous procedure described above for BBX₁₀ starting from (4aS,5aR)-5,5-difluoro-5a-methyl-1,4,4a,5,5a,6-hexahydrocyclopropa[f]indazole-3-carboxylic acid. LCMS: C₃₅H₄₃F₂N₅O₄ requires: 635, found: m/z=636 [M+H]⁺.

### Example 26. 2-{2-[(4aS,5aR)-5,5-difluoro-5a-methyl-1-(oxan-2-yl)-4H,4aH,6H-cyclopropa[f]indazol-3-yl]-1H-indol-6-yl}-2,8-diazaspiro[4.5]decan-1-one (BBX₁₂)

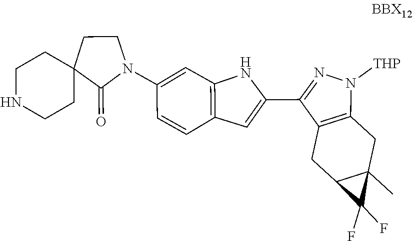

tert-butyl 2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carboxylate (31.00 mg, 0.05 mmol) was stirred in DCM (1.50 mL) and TFA (0.50 mL) for ten minutes. The mixture was concentrated in vacuo to provide 2-{2-[(4aS,5aR)-5,5-difluoro-5a-methyl-1-(oxan-2-yl)-4H,4aH,6H-cyclopropa[f]indazol-3-yl]-1H-indol-6-yl}-2,8-diazaspiro[4.5]decan-1-one (26.8 mg, 100%). LCMS: C₃₀H₃₅F₂N₅O requires: 535, found: m/z=536 [M+H]⁺.

### Example 27. 2-(2-((4aS,5aR)-5,5-difluoro-5a-methyl-1,4,4a,5,5a,6-hexahydrocyclopropa[f]indazol-3-yl)-1H-indol-6-yl)-2,8-diazaspiro[4.5]decan-1-one (BBX₁₃)

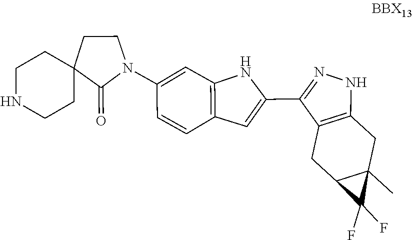 [SMILES: C[C@@]12Cc3[nH]nc(-c4cc5ccc(N6CCC7(CCNCC7)C6=O)cc5[nH]4)c3C[C@@H]1C2(F)F]

To a mixture of tert-butyl 2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carboxylate (52.0 mg, 0.08 mmol) in EtOH (1.00 mL) was added para-toluene sulfonate hydrate (31.1 mg, 0.16 mmol). The mixture was heated at 90° C. for ninety minutes. The mixture was partitioned between dilute aqueous sodium bicarbonate and 20% MeOH:DCM. The aqueous layer was extracted two more times with 20% MeOH:DCM. The combined organic layers were dried over Na₂SO₄ and concentrated to provide 2-{2-[(4aS,5aR)-5,5-difluoro-5a-methyl-1H,4H,4aH,6H-cyclopropa[f]indazol-3-yl]-1H-indol-6-yl}-2,8-diazaspiro[4.5]decan-1-one (0.0350 g, 94.8%). LCMS: C₂₅H₂₇F₂N₅O requires: 451, found: m/z=452 [M+H]⁺.

### Example 28. benzyl 2-(4-(((benzyloxy)carbonyl)(methyl)amino)-2-nitrophenyl)-3-((4aS,5aR)-5,5-difluoro-5a-methyl-1-(tetrahydro-2H-pyran-2-yl)-1,4,4a,5,5a,6-hexahydrocyclopropa[f]indazol-3-yl)-3-oxopropanoate (BBX₁₄)

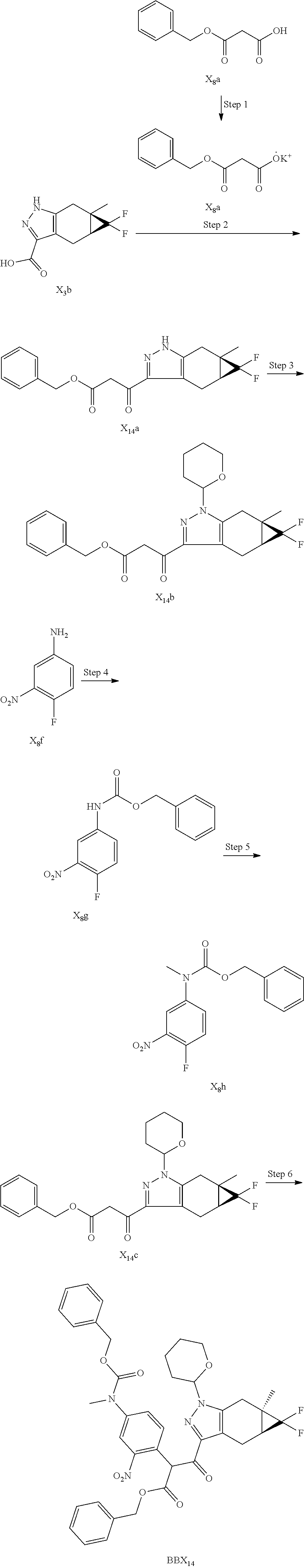

### Step 1: Potassium 3-(benzyloxy)-3-oxopropanoate (X₈a)

A solution of potassium hydroxide (7.37 g, 131 mmol) in anhydrous EtOH (374 mL, 0.35 M) was slowly added to a vigorously stirred solution of malonic acid mono benzyl ester (30 g, 154 mmol) in absolute EtOH (312 mL, 0.42 M) at room temperature and stirred for 2 min. The resulting suspension was diluted with diethyl ether (312 mL, 0.42 M), the precipitate was collected by filtration, and the precipitate was washed with diethyl ether (2×312 mL) to give 29.86 g of potassium mono benzyl malonate as a white solid (84%) yield. ¹H NMR (300 MHz, Deuterium Oxide) δ 7.37 (s, 3H), 7.42-7.26 (m, 2H), 5.12 (s, 2H), 3.26 (s, 2H)

### Step 2: Benzyl 3-[(4aS,5aR)-5,5-difluoro-5a-methyl-1H,4H,4aH,5H,5aH,6H-cyclopropa[f]indazol-3-yl]-3-oxopropanoate (X₁₄a)

To a solution of (4aS,5aR)-5,5-difluoro-5a-methyl-1H,4H,4aH,5H,5aH,6H-cyclopropa[f]indazole-3-carboxylic acid (10.8 g, 47.37 mmol, 1 equiv) in anhydrous THF (108 mL, 0.45 M) was added 1,1′-carbonyldiimidazole (11.51 g, 70.98 mmol, 1.5 equiv) in small portions. The mixture was stirred for one hour at room temperature (caution: CO₂ released). To this mixture was added a solution of potassium mono benzyl malonate (21.98 g, 94.74 mmol, 2.0 equiv) and MgCl₂ (18.024 g, 189.308 mmol, 4.0 equiv) in a mixture of anhydrous THF:anhydrous DMF (1:1) (216 mL, 0.23 M) preheated to 60° C. The reaction mixture was then stirred for 3 h at 60° C. The solvent was evaporated under reduced pressure and the residue was diluted with cold water, neutralized slowly with 10% aq. citric acid solution, and extracted with ethyl acetate. The organic layer was dried over Na₂SO₄ and concentrated under reduced pressure. The residual crude was purified by silica gel flash chromatography (0-50% ethyl acetate in hexane), to give benzyl 3-[(4aS,5aR)-5,5-difluoro-5a-methyl-1H,4H,4aH,5H,5 aH,6H-cyclopropa[f]indazol-3-yl]-3-oxopropanoate (15.0 g, 41.625 mmol, 88%) as a white solid. ¹H NMR (300 MHz, DMSO-d₆) δ 13.24 (s, 1H), 7.43-7.25 (m, 5H), 5.13 (s, 2H), 4.04 (d, J=1.3 Hz, 2H), 3.10-2.96 (m, 3H), 2.81 (dd, J=16.4, 3.4 Hz, 1H), 1.84-1.72 (m, 1H), 1.34 (t, J=2.0 Hz, 3H). LCMS: C₁₉H₁₈F₂N₂O₃ requires: 360, found: m/z=361 [M+H]⁺.

### Step 3: Benzyl 3-[(4aS,5aR)-5,5-difluoro-5a-methyl-1-(oxan-2-yl)-1H,4H,4aH,5H,5aH,6H-cyclopropa[f]indazol-3-yl]-3-oxopropanoate (X₁₄b)

To a solution of benzyl 3-[(4aS,5aR)-5,5-difluoro-5a-methyl-1H,4H,4aH,5H,5aH,6H-cyclopropa[f]indazol-3-yl]-3-oxopropanoate (15.0 g, 41.63 mmol, 1.0 equiv) in ethyl acetate (120.0 mL, 0.35 M) were added p-toluenesulfonic acid monohydrate (0.396 g, 2.08 mmol, 0.05 equiv) and 3,4-dihydro-2H-pyran (5.696 ml, 62.43 mmol, 1.5 equiv). The reaction mixture was stirred for at room temperature for fourteen hours. 5% aq. NaHCO₃ solution (100 mL) was added to the mixture and the mixture was stirred for 10 min. The organic layer was separated and washed with brine (120 mL), dried over Na₂SO₄, and concentrated under reduced pressure. The crude material was purified by flash chromatography (ethyl acetate:hexane (2:8)), to give benzyl 3-[(4aS,5aR)-5,5-difluoro-5a-methyl-1-(oxan-2-yl)-1H,4H,4aH,5H,5 aH,6H-cyclopropa[f]indazol-3-yl]-3-oxopropanoate (17.2 g, 38.697 mmol, 93%) as colorless gummy oil. ¹H NMR (300 MHz, DMSO-d₆) δ 7.43-7.24 (m, 5H), 5.47 (ddd, J=16.0, 9.1, 2.7 Hz, 1H), 5.12 (s, 2H), 3.82 (s, 1H), 3.64 (s, 1H), 3.31-3.16 (m, 1H), 3.15-2.68 (m, 4H), 1.96 (d, J=4.6 Hz, 1H), 1.91-2.02 (m, 1H), 1.89-1.75 (m, 2H), 1.89-1.75 (m, 2H), 1.58 (d, J=28.1 Hz, 4H), 1.36 (s, 3H).

### Step 4: Benzyl N-(4-fluoro-3-nitrophenyl)carbamate (X₈g)

Under a nitrogen atmosphere, sodium hydrogencarbonate (6.45 g, 76.86 mmol, 1.2 equiv) was dissolved in water (150 mL) and ethyl acetate (75 mL), and 4-fluoro-3-nitroaniline (10.0 g, 64 mmol, 1.0 equiv) was then added. Benzyl chloroformate (12.1 g, 70.45 mmol, 1.1 equiv) was then added dropwise at an internal temperature ≤30° C. The mixture was stirred at room temperature for 3 hr, and then left to stand until the layers separated. The organic phase was filtered, washed twice with 5 w/w % brine (150 mL), and concentrated under reduced pressure until the volume was about 20 mL. To this concentrated residue was added heptane (60 mL) at room temperature and the mixture was then stirred 30 min at room temperature. Additional heptane (250 mL) was added, and the mixture was stirred at room temperature for 2 hr. The precipitated crystals were collected by filtration and washed with a mixed solution of ethyl acetate (6 mL) and heptane (54 mL). The wet crystals were dried under reduced pressure to give benzyl 4-fluoro-3-nitrophenylcarbamate (16 g, yield 86%). ¹H NMR (300 MHz, DMSO-d₆) δ 10.28 (s, 1H), 8.34 (dd, J=6.8, 2.8 Hz, 1H), 7.76 (ddd, J=9.1, 3.9, 2.8 Hz, 1H), 7.54 (dd, J=11.2, 9.1 Hz, 1H), 7.48-7.32 (m, 5H), 5.19 (s, 2H).

### Step 5: Benzyl N-(4-fluoro-3-nitrophenyl)-N-methylcarbamate (X₈h)

To a solution of benzyl 4-fluoro-3-nitrophenylcarbamate (16.0 g, 55.125 mmol, 1.0 equiv) in N,N-dimethylformamide (160 mL, 0.57 M) was added cesium carbonate (21.553 g, 66.15 mmol, 1.2 equiv). Methyl iodide (9.389 g, 66.148 mmol, 1.2 equiv) was then added dropwise at room temperature and the mixture was stirred at room temperature for 3 hr. Toluene (160 mL) and water (80 mL) were then added and this solution was stirred for about 10 min. The mixture was then left to stand until the layers separated. The organic layer was washed twice with water (100 mL), and concentrated under reduced pressure until the volume was about 50 mL. To this concentrated volume was added heptane (40 mL) and the mixture was stirred at room temperature for 30 min. Additional heptane (60 mL) was added, and the mixture was stirred for one hour at room temperature. The precipitated crystals were collected by filtration, and the obtained wet crystals were washed with a mixed solution of toluene (3 mL) and heptane (25 mL). The wet crystals were then dried under reduced pressure to give benzyl N-(4-fluoro-3-nitrophenyl)-N-methylcarbamate (16.0 g, 52.584 mmol, 95% yield). ¹H NMR (300 MHz, DMSO-d₆) δ 8.19 (dd, J=6.7, 2.8 Hz, 1H), 7.82 (ddd, J=9.0, 4.0, 2.8 Hz, 1H), 7.60 (dd, J=11.1, 9.0 Hz, 1H), 7.35 (d, J=3.8 Hz, 5H), 5.15 (s, 2H), 3.30 (s, 3H).

### Step 6: benzyl 2-(4-(((benzyloxy)carbonyl)(methyl)amino)-2-nitrophenyl)-3-((4aS,5aR)-5,5-difluoro-5a-methyl-1-(tetrahydro-2H-pyran-2-yl)-1,4,4a,5,5a,6-hexahydrocyclopropa[f]indazol-3-yl)-3-oxopropanoate (BBX₁₄)

To a solution of benzyl 3-[(4aS,5aR)-5,5-difluoro-5a-methyl-1-(oxan-2-yl)-1H,4H,4aH,5H,5aH,6H-cyclopropa[f]indazol-3-yl]-3-oxopropanoate (6.0 g, 13.499 mmol, 1.0 equiv) in anhydrous dimethyl sulfoxide (60.0 mL, 0.22 M) were successively added benzyl N-(4-fluoro-3-nitrophenyl)-N-methylcarbamate (4.929 g, 16.199 mmol, 1.2 equiv) and potassium carbonate (3.731 g, 26.996 mmol, 2.0 equiv). The mixture was stirred in an oil bath at 90° C. for 2 hr and 20 min. The reaction mixture was then cooled to room temperature, poured into 5% aq. citric acid solution (100 mL), and extracted with ethyl acetate. The organic layer was washed with water, brine, dried over anhydrous sodium sulfate, and concentrated under reduced pressure. The obtained residue was purified by silica gel flash chromatography (ethyl acetate:hexane 2:1), to give the title product as a light yellow foamy solid (8.24 g, 84% yield). ¹H NMR (300 MHz, DMSO-d₆) δ 8.21-8.01 (m, 1H), 7.74 (ddt, J=9.4, 6.4, 2.8 Hz, 1H), 7.53-7.13 (m, 10H), 6.37-6.22 (m, 1H), 5.44 (dd, J=16.9, 8.0 Hz, 1H), 5.31-5.08 (m, 4H), 3.74 (s, 1H), 3.68-3.48 (m, 2H), 3.23-3.12 (m, 1H), 3.00 (s, 3H), 2.76 (s, 1H), 2.03 (s, 1H), 1.78 (t, J=15.4 Hz, 3H), 1.51 (s, 3H), 1.34 (s, 3H). LCMS: C₃₉H₃₈F₂N₄O₈ requires: 728, found: m/z=729 [M+H]⁺.

### Example 29. 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-N-methyl-1H-indol-6-amine (BBX₁₅)

Synthesis of BBX₁₅ is shown in Step 2 of Example 30 below.

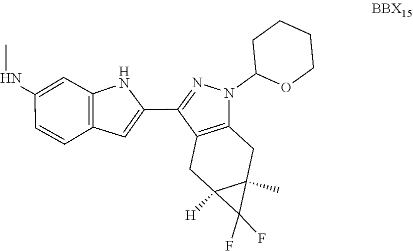 [SMILES: CNc1ccc2cc(-c3nn(C4CCCCO4)c4c3C[C@@H]3C(F)(F)[C@]3(C)C4)[nH]c2c1]

### Example 30. (S)—N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperazin-1-yl)propenamide (BBX₁₆)

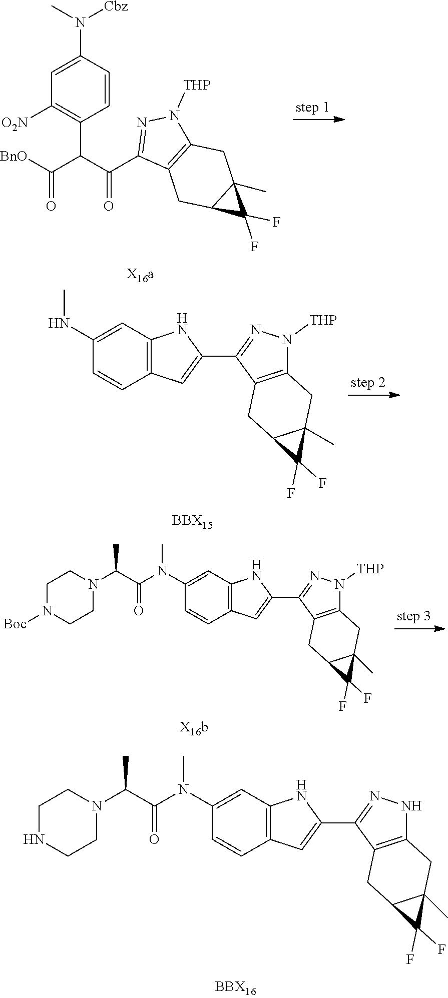

### Step 1: 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-N-methyl-1H-indol-6-amine (BBX₁₅)

A mixture of benzyl N-{2-[(4aS,5aR)-5,5-difluoro-5a-methyl-1-(oxan-2-yl)-4H,4aH,6H-cyclopropa[f]indazol-3-yl]-1H-indol-6-yl}-N-methylcarbamate (46 mg, 0.08 mmol) and 10% Pd/C (5 mg) in EtOH (1 mL) and toluene (1 mL) was stirred under a balloon of H₂. After three hours, 10% Pd/C (5 mg) was added. After four hours the mixture was diluted with THF, filtered through celite, and toluene (5 mL) was added. The mixture was concentrated to about one milliliter and toluene (5 mL) was added. The mixture was then concentrated to about one milliliter and taken into the next step without further purification as a solution of 2-[(4aS,5aR)-5,5-difluoro-5a-methyl-1-(oxan-2-yl)-4H,4aH,6H-cyclopropa[f]indazol-3-yl]-N-methyl-1H-indol-6-amine in toluene. LCMS: C₂₃H₂₆F₂N₄O requires: 412, found: m/z=413 [M+H]⁺.

### Step 2: tert-butyl 4-((2S)-1-((2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)(methyl)amino)-1-oxopropan-2-yl)piperazine-1-carboxylate (X₁₆b)

To a mixture of (2S)-2-[4-(tert-butoxycarbonyl)piperazin-1-yl]propanoic acid (21 mg, 0.08 mmol) in DMF (1 mL) was added HATU (32 mg, 0.08 mmol) and N,N-diisopropylethylamine (0.06 mL, 0.34 mmol). After five minutes, the mixture was added to a solution of 2-[(4aS,5aR)-5,5-difluoro-5a-methyl-1-(oxan-2-yl)-4H,4aH,6H-cyclopropa[f]indazol-3-yl]-N-methyl-1H-indol-6-amine (35 mg, 0.08 mmol) in toluene (1 mL). After stirring overnight, the mixture was transferred to a separatory funnel containing ethyl acetate. The resulting solution was washed with water, and brine was added to get the layers to separate. The organic layer was dried over Na₂SO₄ and concentrated. The crude residue was purified by flash chromatography on a 12 g column eluted with 0 to 5% MeOH:DCM to provide tert-butyl 4-[(1S)-1-({2-[(4aS,5aR)-5,5-difluoro-5a-methyl-1-(oxan-2-yl)-4H,4aH,6H-cyclopropa[f]indazol-3-yl]-1H-indol-6-yl}(methyl)carbamoyl)ethyl]piperazine-1-carboxylate (0.052 g, quant.). LCMS: C₃₅H₄₆F₂N₆O₄ requires: 652, found: m/z=653 [M+H]⁺.

### Step 3: (S)—N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperazin-1-yl)propanamide (BBX₁₆)

Following the procedure described at Step 6, in the above procedure for BBX₉, and starting from tert-butyl 4-((2S)-1-((2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)(methyl)amino)-1-oxopropan-2-yl)piperazine-1-carboxylate (600 mg, 0.919 mmol) provided (S)—N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperazin-1-yl)propanamide (150 mg, 0.321 mmol, 35%).

### Example 31. N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperidin-4-yl)acetamide (BBX₁₇)

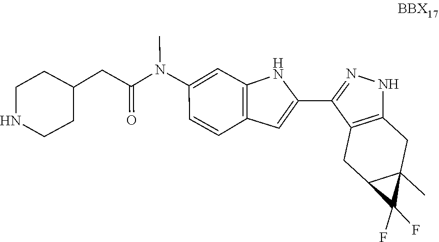 [SMILES: CN(C(=O)CC1CCNCC1)c1ccc2cc(-c3n[nH]c4c3C[C@@H]3C(F)(F)[C@]3(C)C4)[nH]c2c1]

Following the procedure for BBX₁₆ and starting from [1-(tert-butoxycarbonyl)piperidin-4-yl]acetic acid (50 mg, 0.21 mmol) provided N-{2-[(4aS,5aR)-5,5-difluoro-5a-methyl-1H,4H,4aH,6H-cyclopropa[f]indazol-3-yl]-1H-indol-6-yl}-N-methyl-2-(piperidin-4-yl)acetamide (BBX₁₇) (0.082 g, quant.). LCMS: C₂₅H₂₉F₂N₅O requires: 453.2, found: m/z=454.4 [M+H]⁺.

### Example 32. N-{2-[(4aS,5aR)-5,5-difluoro-5a-methyl-1H,4H,4aH,6H-cyclopropa[f]indazol-3-yl]-1H-indol-6-yl}-N-methylpiperidine-4-carboxamide (BBX₁₈)

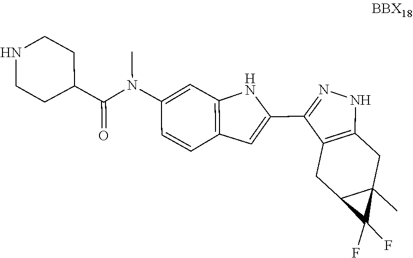 [SMILES: CN(C(=O)C1CCNCC1)c1ccc2cc(-c3n[nH]c4c3C[C@@H]3C(F)(F)[C@]3(C)C4)[nH]c2c1]

Following the procedure for BBX₁₄ and starting from 1-(tert-butoxycarbonyl)piperidine-4-carboxylic acid (72 mg, 0.31 mmol) provided N-{2-[(4aS,5aR)-5,5-difluoro-5a-methyl-1H,4H,4aH,6H-cyclopropa[f]indazol-3-yl]-1H-indol-6-yl}-N-methylpiperidine-4-carboxamide (0.072 g, quant.). LCMS: C₂₄H₂₇F₂N₅O requires: 439.2, found: m/z=440.4 [M+H]⁺.

### Example 33. 2-(6,6-dimethyl-4,5,6,7-tetrahydro-1H-indazol-3-yl)-1H-indole-6-carboxylic acid (BBX₁₉)

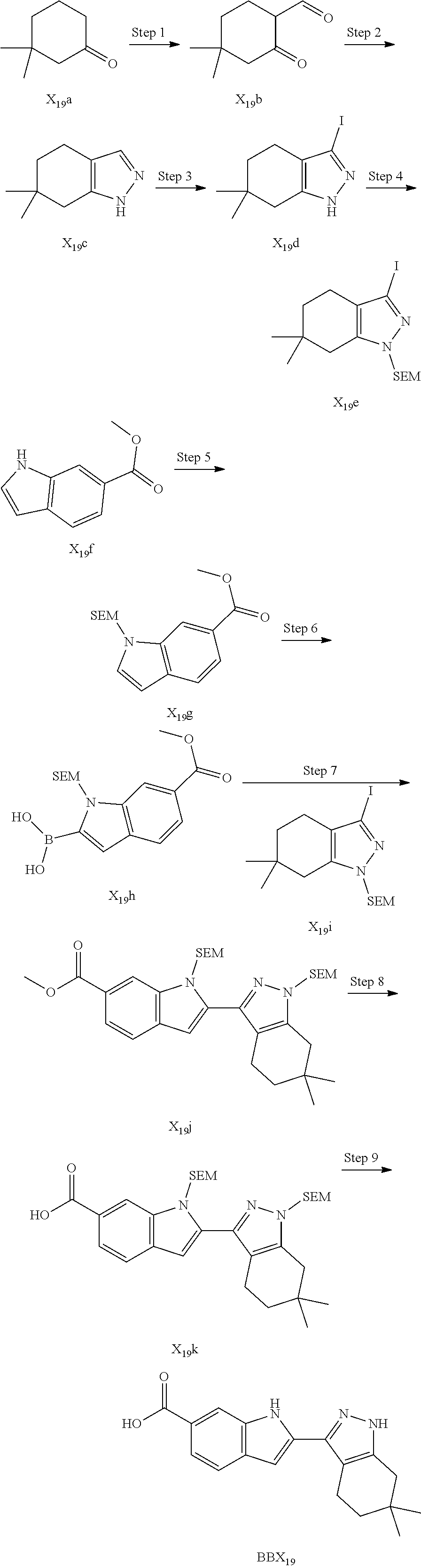

### Step 1: 4,4-Dimethyl-2-oxocyclohexanecarboxaldehyde (X₁₉b)

To a suspension of NaH (17.431 g, 436 mmol, 1.1 equiv) in THF (310 mL), under nitrogen atmosphere, was added a solution of 3,3-dimethylcyclohexanone (50.0 g, 396 mmol, 1.0 equiv) in THF (160 mL) dropwise at 0° C. over about one hour, and the mixture was stirred for one hour. Then, a solution of ethyl formate (60 g, 812 mmol, 2.05 equiv) in THF (160 mL) was added dropwise at 0° C. for one hour and stirred for another two hours from 0° C. to room temperature. At completion, to the reaction mixture was added water and diethyl ether, and the organic layer was separated and extracted with 2 N NaOH solution. The aqueous layer was acidified with concentrated HCl and extracted with diethyl ether. Then, the organic layer was washed with brine, dried over sodium sulfate, filtered, and concentrated under reduced pressure to afford the title compound (50 g, 82% yield). ¹H NMR (300 MHz, DMSO-d₆) δ 11.42 (s, 1H), 10.01-8.98 (m, 1H), 2.19 (d, J=7.7 Hz, 2H), 2.06 (s, 2H), 1.37 (t, J=6.7 Hz, 2H), 0.91 (s, 6H).

### Step 2: 6,6-dimethyl-4,5,6,7-tetrahydro-1H-indazole (X₁₉c)

To a solution of 4,4-dimethyl-2-oxocyclohexanecarboxaldehyde (50.0 g, 324 mmol, 1.0 equiv) in MeOH (235 mL) was added a solution of hydrazine hydrate (25 mL, 327 mmol, 1.01 equiv) in MeOH (20 mL) dropwise with heating under reflux over one hour and the mixture was then stirred for 15 min. The reaction mixture was concentrated under reduced pressure, ethyl acetate and water were added, and the organic layer was separated. Then the organic layer was washed with brine, dried over sodium sulfate, filtered, and concentrated under reduced pressure to afford the title compound (46.6 g, 96% yield). ¹H NMR (300 MHz, DMSO-d₆) δ 12.20 (s, 1H), 7.25 (s, 1H), 2.61-2.35 (m, 4H), 1.44 (t, J=6.5 Hz, 2H), 1.18 (t, J=7.1 Hz, 1H), 0.94 (s, 6H).

### Step 3: 3-iodo-6,6-dimethyl-4,5,6,7-tetrahydro-1H-indazole (X₁₉d)

To a solution of 6,6-dimethyl-4,5,6,7-tetrahydro-1H-indazole (50 g, 333 mmol, 1.0 equiv) in DMF (950 mL, 0.35 M) was added iodine (212 g, 835 mmol, 2.5 equiv) and KOH (92 g, 1647 mmol, 4.95 equiv) at rt and the mixture was stirred for 4 h. At completion, the reaction was cooled in an ice bath and an aqueous solution of sodium hydrogen sulfite (25 g in 250 mL water) was added dropwise. More water (625 mL) was then added. A precipitate appeared and was filtered to obtain the title compound (56 g, 65% yield). ¹H NMR (300 MHz, DMSO-d₆) δ 12.69 (s, 1H), 2.33 (s, 2H), 2.21 (t, J=6.4 Hz, 2H), 1.47 (t, J=6.4 Hz, 2H), 0.94 (s, 6H).

### Step 4: 3-iodo-6,6-dimethyl-1-{[2-(trimethylsilyl)ethoxy]methyl}-4,5,6,7-tetrahydro-1H-indazole (X₁₉e)

To a solution of 3-iodo-6,6-dimethyl-4,5,6,7-tetrahydro-1H-indazole (56 g, 203 mmol, 1.0 equiv) in DMF (450 mL, 0.45 M), under an argon atmosphere, was added NaH (8.9 g, 223 mmol, 1.1 equiv) in 500 mg portions at an internal temperature of −10° C. The mixture was then stirred for 30 min. Then, (2-(Chloromethoxy)ethyl)trimethylsilane (35.9 mL, 203 mmol, 1.0 equiv) was added dropwise over 15 min, and the mixture was further stirred for 2 h. At completion, ethyl acetate and water were added, the organic layer was separated, washed with ice-cold water and brine, dried over sodium sulfate, filtered, and concentrated under reduced pressure to afford the title compound (71.5 g, 97% yield). ¹H NMR (300 MHz, DMSO-d₆) δ 5.30 (d, J=17.8 Hz, 2H), 3.49 (dt, J=15.7, 7.9 Hz, 2H), 2.40 (s, 1H), 2.31 (s, 1H), 2.30-2.16 (m, 2H), 1.48 (q, J=6.2 Hz, 2H), 0.94 (d, J=6.5 Hz, 6H), 0.79 (t, J=7.9 Hz, 2H), −0.06 (d, J=1.2 Hz, 9H).

### Step 5: methyl 1-{[2-(trimethylsilyl)ethoxy]methyl}-1H-indole-6-carboxylate (X₁₉g)

To a solution of methyl 1H-indole-6-carboxylate (60 g, 342.5 mmol, 1.0 equiv) in DMF (685 mL, 0.5 M), under an argon atmosphere, was added NaH (14.5 g, 376.8 mmol, 1.1 equiv) in portions at an internal temperature of −10° C., and the mixture was then stirred for one hour. Then, (2-(Chloromethoxy)ethyl)trimethylsilane (60.6 ml, 342.5 mmol, 1.0 equiv) was added dropwise over 1h, and the mixture was further stirred overnight. At completion, ethyl acetate and water were added to the reaction mixture, and the organic layer was separated. Then, the organic layer was washed with brine, dried over sodium sulfate, filtered, and concentrated under reduced pressure. The residue was used in the next step without purification. ¹H NMR (300 MHz, DMSO-d₆) δ 8.21 (s, 1H), 7.76-7.65 (m, 3H), 6.61-6.55 (m, 1H), 5.64 (s, 2H), 3.87 (s, 3H), 3.50-3.38 (m, 2H), 0.87-0.74 (m, 2H), −0.11 (s, 9H).

### Step 6: [6-(methoxycarbonyl)-1-{[2-(trimethylsilyl)ethoxy]methyl}-1H-indol-2-yl]boronic acid (X₁₉h)

To a solution of methyl 1-{[2-(trimethylsilyl)ethoxy]methyl}-1H-indole-6-carboxylate (20 g, 65.5 mmol, 1.0 equiv) in THF (66 mL, 1 M) was added triisopropylborate (23 mL, 19 mmol, 1.5 equiv), and the reaction mixture was then cooled to −10° C. Then, LDA (2 M solution in hexane, 44.2 mL, 88.4 mmol, 1.35 equiv) was added dropwise over 20 min while maintaining the internal temperature at −10° C. The mixture was further stirred for one hour. Then, the reaction mixture was cooled in an ice bath and 10% aqueous NaHCO₃ solution was added dropwise until pH 9. The aqueous layer was extracted three times with ethyl acetate, and the combined organic layers were washed with brine, dried over magnesium sulfate, filtered, and the filtrate was concentrated under reduced pressure. The crude material was used in the next step without further purification.

### Step 7: methyl 2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carboxylate (X₁₉i)

Under an argon atmosphere, 3-iodo-6,6-dimethyl-1-{[2-(trimethylsilyl)ethoxy]methyl}-4,5,6,7-tetrahydro-1H-indazole (36.7 g, 90.4 mmol, 1.0 equiv), potassium phosphate tribasic (76.74 g, 361.5 mmol, 4 equiv), and Pd(dppf)Cl₂.DCM complex (7.42 g, 9.04 mmol, 0.1 equiv) were mixed in a mixture of dioxane (532 mL) and water (226 mL). Then, [6-(methoxycarbonyl)-1-{[2-(trimethylsilyl)ethoxy]methyl}-1H-indol-2-yl]boronic acid (38 g, 108.5 mmol, 1.2 equiv) was added in portions with heating at 110° C. over 15 min, and the mixture was stirred for another 5 min. After cooling, water and ethyl acetate were added to the reaction mixture, and the organic layer was separated. Then, the organic layer was washed with water and saturated brine, dried over sodium sulfate, filtered, and concentrated under reduced pressure. The residue was purified by flash chromatography (5% to 15% ethyl acetate in hexane) to give the title compound (28 g, 48% yield over three steps). ¹H NMR (300 MHz, DMSO-d₆) δ 8.22 (s, 1H), 7.74-7.64 (m, 2H), 6.83 (s, 1H), 6.14 (s, 2H), 5.42 (s, 2H), 3.88 (s, 3H), 3.66-3.52 (m, 4H), 2.68-2.56 (m, 2H), 1.54 (t, J=5.9 Hz, 2H), 1.18 (t, J=7.1 Hz, 2H), 1.03 (s, 6H), 0.92-0.83 (m, 2H), 0.73-0.63 (m, 2H), −0.03 (s, 9H), −0.21 (s, 9H).

### Step 8: 2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carboxylic acid (X₁₉j)

To a solution of methyl 2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carboxylate (16 g, 27.4 mmol, 1.0 equiv) in a mixture of THF (80 mL) and MeOH (80 mL) was added 4N NaOH (34.2 mL, 137 mmol, 5 equiv), and the mixture was heated at 60° C. for 2 h. After cooling, the reaction mixture was concentrated, and 10% aqueous citric acid solution was added to the residue to adjust the pH to five. The mixture was extracted with ethyl acetate, washed with brine, dried over sodium sulfate, filtered, and concentrated under reduced pressure. The compound was purified via flash chromatography (hexane:ethyl acetate 8:2) to give the title compound (13.4 g, 85% yield). ¹H NMR (300 MHz, DMSO-d₆) δ 12.63 (s, 1H), 8.22 (s, 1H), 7.74-7.64 (m, 2H), 6.83 (s, 1H), 6.14 (s, 2H), 5.42 (s, 2H), 3.66-3.52 (m, 4H), 2.68-2.56 (m, 2H), 1.54 (t, J=5.9 Hz, 2H), 1.18 (t, J=7.1 Hz, 2H), 1.03 (s, 6H), 0.92-0.83 (m, 2H), 0.73-0.63 (m, 2H), −0.03 (s, 9H), −0.21 (s, 9H). LCMS: C₃₀H₄₇N₃O₄Si₂ requires: 569, found: m/z=570 [M+H]⁺.

### Step 9: 2-(6,6-dimethyl-4,5,6,7-tetrahydro-1H-indazol-3-yl)-1H-indole-6-carboxylic acid (BBX₁₉)

Bu₄NF (1.0 M in THF, 2.63 mL, 2.63 mmol) and ethylenediamine (528 μL, 7.9 mmol) was added to a solution of 2-(6,6-dimethyl-1-{[2-(trimethylsilyl)ethoxy]methyl}-5,7-dihydro-4H-indazol-3-yl)-1-{[2-(trimethylsilyl)ethoxy]methyl}indole-6-carboxylic acid (300 mg, 0.53 mmol) in DME (2.65 mL). The reaction mixture was stirred at 90° C. for 16 h, and then was concentrated under reduced pressure. The residue was dissolved in EtOAc, and acidified with 10% aqueous citric acid (pH ˜4-5). The aqueous layer was extracted with EtOAc. The combined organic layer was dried over Na₂SO₄, and concentrated under reduced pressure. Flash chromatography (SiO₂, 0-100% H₂O:MeCN gradient elution) afforded the desired product (87.4 mg, 0.28 mmol, 54%). LCMS: C₁₈H₁₉N₃O₂ requires: 309, found: m/z=310 [M+H]⁺.

### Example 34. (2-(6,6-dimethyl-4,5,6,7-tetrahydro-1H-indazol-3-yl)-1H-indol-6-yl)(piperazin-1-yl)methanone (BBX₂₀)

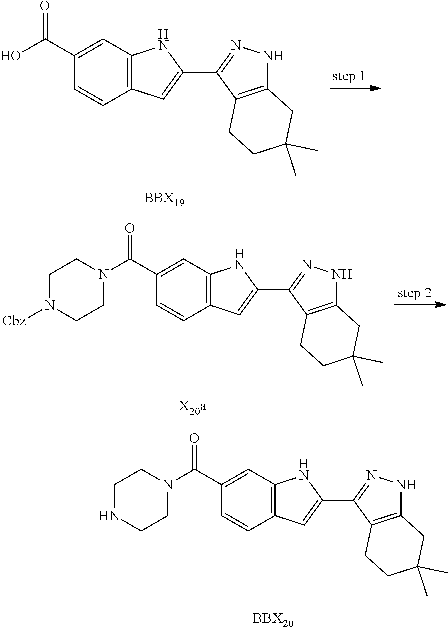 [SMILES: CC1(C)CCc2c(-c3cc4ccc(C(=O)O)cc4[nH]3)n[nH]c2C1 ; CC1(C)CCc2c(-c3cc4ccc(C(=O)N5CCN(C(=O)OCc6ccccc6)CC5)cc4[nH]3)n[nH]c2C1 ; CC1(C)CCc2c(-c3cc4ccc(C(=O)N5CCNCC5)cc4[nH]3)n[nH]c2C1]

### Step 1: Benzyl 4-[2-(6,6-dimethyl-4,5,6,7-tetrahydro-1H-indazol-3-yl)-1H-indole-6-carbonyl]piperazine-1-carboxylate (X₂₀a)

To a solution of 2-(6,6-dimethyl-4,5,6,7-tetrahydro-1H-indazol-3-yl)-1H-indole-6-carboxylic acid (1 g, 3.235 mmol), i-Pr₂NEt (1.69 mL, 9.702 mmol), benzyl 1-piperazinecarboxylate (0.624 ml, 3.235 mmol) in DMF (16 mL) was added HATU (1.845 g, 4.852 mmol) in DMF (5 mL) at 0° C. After stirred at room temperature for 1 hour, the reaction mixture was poured onto crushed ice and extracted with EtOAc. The combined organic layers were dried over Na₂SO₄, and concentrated under reduced pressure. Flash chromatography using EtOAc:hexanes (4:6) afforded the desired product (0.92 g, 1.8 mmol, 56%). ¹H NMR (300 MHz, (CD₃)₂SO) δ 12.58 (s, 1H), 11.48 (s, 1H), 7.54 (d, J=8.1 Hz, 1H), 7.47 (s, 1H), 7.41-7.28 (m, 5H), 7.03 (d, J=7.8 Hz, 1H), 6.62 (s, 1H), 5.11 (s, 2H), 3.51 (d, J=20.5 Hz, 8H), 2.68 (d, J=4.5 Hz, 2H), 2.42 (s, 2H), 1.58 (t, J=6.2 Hz, 2H), 1.01 (s, 6H). LCMS: C₃₀H₃₃N₅O₃ requires: 511, found: m/z=512 [M+H]⁺.

### Step 2: (2-(6,6-dimethyl-4,5,6,7-tetrahydro-1H-indazol-3-yl)-1H-indol-6-yl)(piperazin-1-yl)methanone (BBX₂₀)

A solution of benzyl 4-[2-(6,6-dimethyl-1,4,5,7-tetrahydroindazol-3-yl)-1H-indole-6-carbonyl]piperazine-1-carboxylate (250 mg, 0.49 mmol) in MeOH (5 mL) and DCM (5 mL) was stirred under H₂ (1 atm) in the presence of Pd/C (50 mg) for one hour. The reaction mixture was filtered through Celite, and concentrated under reduced pressure to afford the desired product (104 mg, 0.27 mmol, 55%). LCMS: C₂₂H₂₇N₅O requires: 377, found: m/z=378 [M+H]⁺.

### Example 35. (2-(6,6-dimethyl-4,5,6,7-tetrahydro-1H-indazol-3-yl)-1H-indol-6-yl)(piperazin-1-yl)methanone (BBX₂₁)

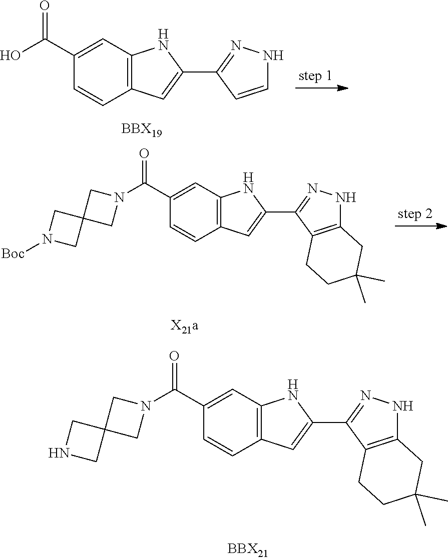 [SMILES: O=C(O)c1ccc2cc(-c3cc[nH]n3)[nH]c2c1 ; CC1(C)CCc2c(-c3cc4ccc(C(=O)N5CC6(CN(C(=O)OC(C)(C)C)C6)C5)cc4[nH]3)n[nH]c2C1 ; CC1(C)CCc2c(-c3cc4ccc(C(=O)N5CC6(CNC6)C5)cc4[nH]3)n[nH]c2C1]

### Step 1: tert-butyl 6-[2-(6,6-dimethyl-1,4,5,7-tetrahydroindazol-3-yl)-1H-indole-6-carbonyl]-2,6-diazaspiro[3.3]heptane-2-carboxylate (X₂₁a)

BOP (61.9 mg, 0.14 mmol) was added to a mixture of 2-(6,6-dimethyl-1,4,5,7-tetrahydroindazol-3-yl)-1H-indole-6-carboxylic acid (43.3 mg, 0.14 mmol), tert-butyl 2,6-diazaspiro[3.3]heptane-2-carboxylate (27.8 mg, 0.14 mmol), and N,N-diisopropylethylamine (0.07 mL, 0.05 g, 0.42 mmol) in DMF (0.70 mL). After stirring overnight, water (1 mL) was added. The mixture was extracted twice with DCM (1 mL). The combined organic layers were concentrated to provide tert-butyl 6-[2-(6,6-dimethyl-1,4,5,7-tetrahydroindazol-3-yl)-1H-indole-6-carbonyl]-2,6-diazaspiro[3.3]heptane-2-carboxylate (68 mg, 100%). LCMS: C₂₈H₃₅N₅O₃ requires: 317, found: m/z=318 [M+H]⁺.

### Step 2: 3-(6-{2,6-diazaspiro[3.3]heptane-2-carbonyl}-1H-indol-2-yl)-6,6-dimethyl-1,4,5,7-tetrahydroindazole (BBX₂₁)

tert-butyl 6-[2-(6,6-dimethyl-1,4,5,7-tetrahydroindazol-3-yl)-1H-indole-6-carbonyl]-2,6-diazaspiro[3.3]heptane-2-carboxylate (68.6 mg, 0.14 mmol) was stirred in DCM (0.50 mL) and TFA (0.50 mL) for fifteen minutes. The mixture was then concentrated to provide 3-(6-{2,6-diazaspiro[3.3]heptane-2-carbonyl}-1H-indol-2-yl)-6,6-dimethyl-1,4,5,7-tetrahydroindazole (52 mg, 0.14 mmol). LCMS: C₂₃H₂₇N₅O requires 389, found: m/z=390 [M+H]⁺.

The following General Procedures Schemes 1-4 illustrate the bond formations by which the CRBN harnesses may be coupled with target hooks to afford the CRBN-based CTMs of name and structure.

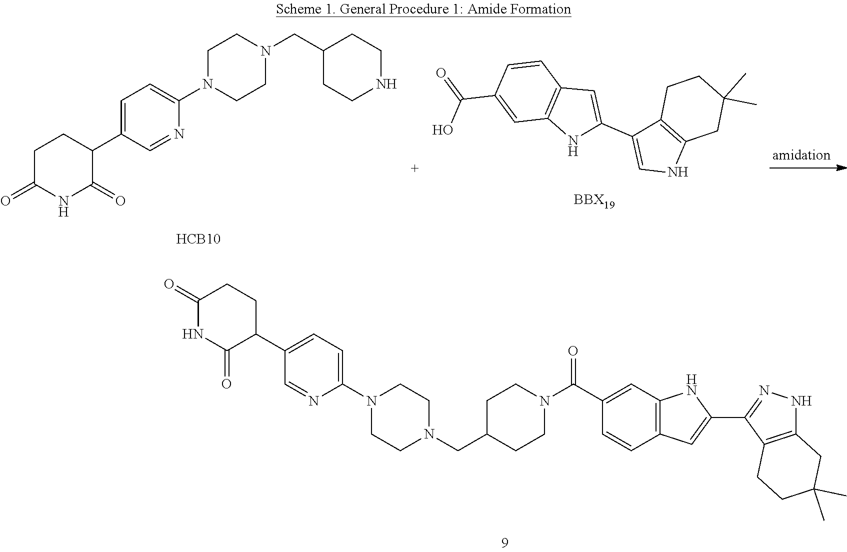

### Scheme 1: Synthesis of Compound 1 Via Amide Formation

A mixture of carboxylic acid (29 mg, 0.09 mmol), amine (29 mg, 0.08 mmol), BOP (41.5 mg, 0.09 mmol), and i-Pr₂NEt (76.1 μL, 0.39 mmol) in DMF (400 μL) was allowed to stir at room temperature for 16 h. The reaction mixture was purified by HPLC (H₂O:MeCN with 0.1% TFA) to afford the desired product (14 mg, 0.02 mmol, 27%). %). An exemplary amide coupling is provided in Scheme 1 above where 2-(6,6-dimethyl-4,5,6,7-tetrahydro-1H-indazol-3-yl)-1H-indole-6-carboxylic acid (BBX₁₉) was treated with 3-(6-(4-(piperidin-4-ylmethyl)piperazin-1-yl)pyridin-3-yl)piperidine-2,6-dione (HCB10) to afford 3-(6-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-3-yl)piperidine-2,6-dione (Compound 9). ¹H NMR (500 MHz, (CD₃)₂SO) δ 11.45 (s, 1H), 10.84 (s, 1H), 9.34 (s, 1H), 8.03 (d, J=2.4 Hz, 1H), 7.56-7.49 (m, 2H), 7.44 (s, 1H), 7.02-6.94 (m, 2H), 6.62 (d, J=2.0 Hz, 1H), 4.35 (d, J=13.7 Hz, 2H), 3.79 (dd, J=12.4, 4.9 Hz, 1H), 3.61 (d, J=11.8 Hz, 2H), 3.21 (t, J=12.7 Hz, 2H), 3.10 (dd, J=18.2, 8.2 Hz, 4H), 2.73-2.62 (m, 3H), 2.54 (s, 1H), 2.50-2.44 (m, 3H), 2.42 (s, 2H), 2.19 (ddd, J=25.6, 13.9, 8.0 Hz, 2H), 2.00-1.93 (m, 1H), 1.77 (d, J=19.2 Hz, 2H), 1.58 (t, J=6.4 Hz, 2H), 1.22 (d, J=13.4 Hz, 3H), 1.01 (s, 6H). LCMS: C₃₈H₄₆F₂N₈O₃ requires: 662, found: m/z=663 [M+H]⁺.

HATU or BOP was typically used as a coupling reagent, but other coupling agents have been contemplated as would be appreciated by a person having skill in the art.

Other amine containing compounds of this disclosure that were synthesized by using General Procedure 1 are Compounds 1-3, 10, 11, 19, 33, 35, 37-39, 41, and 42.

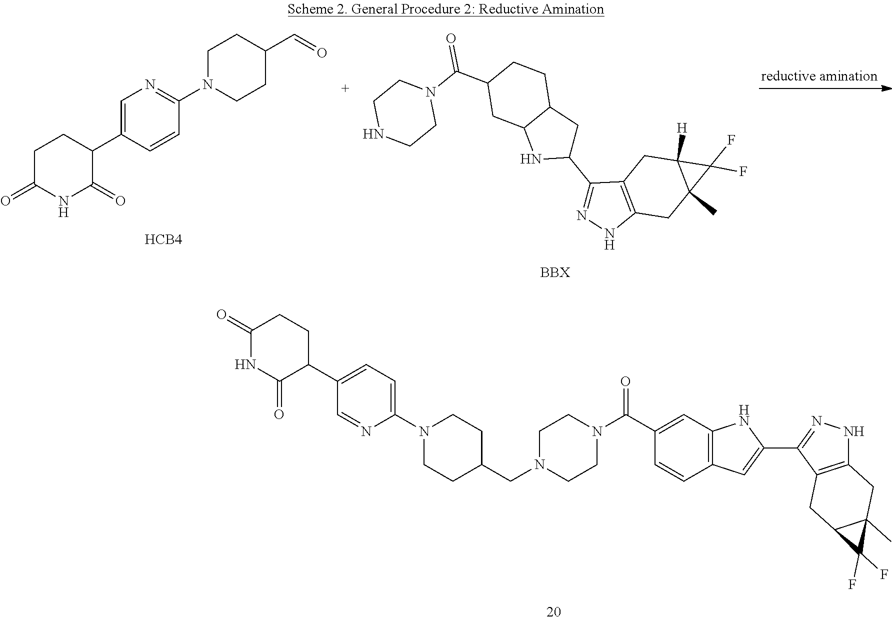

### Scheme 2: Synthesis of Compound 20 Via Reductive Amination

A mixture of amine (20 mg, 0.05 mmol), aldehyde (15 mg, 0.05 mmol), and Et₃N (34 μL, 0.11 mmol) in DCE (0.50 mL) was allowed to stir at rt for 10 min. NaBH(OAc)₃ (16.4 mg, 0.24 mmol) was added, and the reaction mixture was allowed to stir at rt for one hour. The reaction was quenched with H₂O, extracted with 10% MeOH in DCM, concentrated, and purified by HPLC (H₂O:MeCN with 0.1% TFA) to afford the amine product (16 mg, 0.02 mmol, 48%). An exemplary reductive amination is provided in Scheme 2 where (2-((4aS,5aR)-5,5-difluoro-5a-methyl-1,4,4a,5,5a,6-hexahydrocyclopropa[f]indazol-3-yl)-1H-indol-6-yl)(piperazin-1-yl)methanone (BBX₂₂) was treated with 1-(5-(2,6-dioxopiperidin-3-yl)pyridin-2-yl)piperidine-4-carbaldehyde (HCB4) as described above to provide 3-(6-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)pyridin-3-yl)piperidine-2,6-dione (Compound 20). ¹H NMR (500 MHz, CD₃CN) δ 10.01 (d, J=30.6 Hz, 1H), 8.87 (s, 1H), 7.84-7.77 (m, 2H), 7.64 (d, J=8.1 Hz, 1H), 7.55 (s, 1H), 7.18 (d, J=9.4 Hz, 1H), 7.14 (dd, J=8.2, 1.4 Hz, 1H), 6.72 (d, J=3.5 Hz, 1H), 4.10 (d, J=13.5 Hz, 2H), 3.80 (dd, J=12.8, 5.1 Hz, 1H), 3.53 (s, 3H), 3.25-3.06 (m, 5H), 3.00 (d, J=6.8 Hz, 2H), 2.82 (dd, J=16.8, 3.3 Hz, 1H), 2.70-2.64 (m, 2H), 2.30-2.05 (m, 2H), 2.03-1.96 (m, 2H), 1.81-1.74 (m, 1H), 1.41 (s, 5H). LCMS: C₃₈H₄₂F₂N₈O₃ requires: 696, found: m/z=697 [M+H]⁺.

DCM was typically used as a solvent. Et₃N or i-Pr₂NEt was typically used as a base.

Other amine containing compounds of this disclosure that were synthesized by using General Procedure 2 are Compounds 8, 15, and 26.

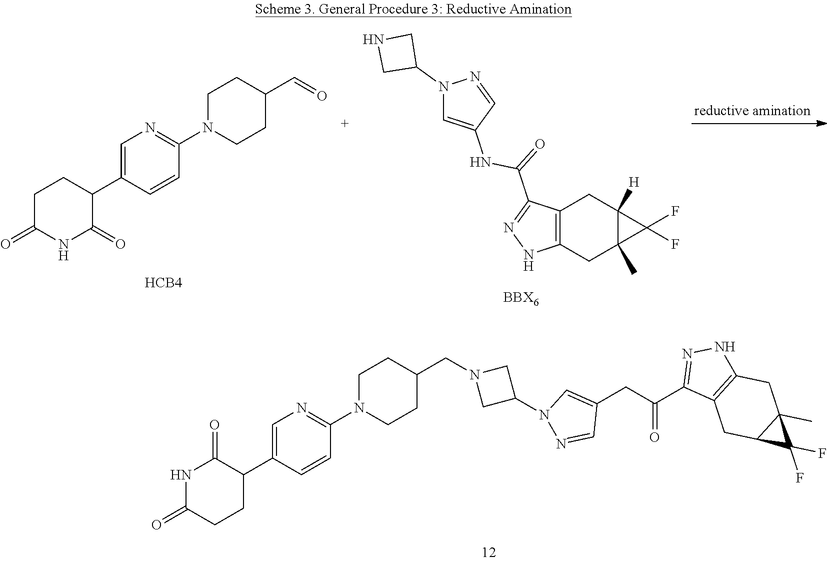

### Scheme 3: Synthesis of Compound 12 Via Reductive Amination

A mixture of amine (20 mg, 0.06 mmol), aldehyde (17 mg, 0.06 mmol), and Et₃N (40.9 μL, 0.29 mmol) in DCE (0.6 mL) was allowed to stir at rt for 10 min. NaBH(OAc)₃ (0.37 mg, 0.17 mmol) was added, and the reaction mixture was allowed to stir at rt for one hour. The reaction was then quenched with H₂O, extracted with DCM, concentrated, and purified by HPLC (H₂O:MeCN with 0.1% TFA) to afford the amine product (23 mg, 0.04 mmol, 64%). An exemplary reductive amination is provided in Scheme 3 where (4aS,5aR)—N-(1-(azetidin-3-yl)-1H-pyrazol-4-yl)-5,5-difluoro-5a-methyl-1,4,4a,5,5a,6-hexahydrocyclopropa[f]indazole-3-carboxamide (BBX₆) was treated with 1-(5-(2,6-dioxopiperidin-3-yl)pyridin-2-yl)piperidine-4-carbaldehyde (HCB4) as described above to provide (4aS,5aR)—N-(1-(1-((1-(5-(2,6-dioxopiperidin-3-yl)pyridin-2-yl)piperidin-4-yl)methyl)azetidin-3-yl)-1H-pyrazol-4-yl)-5,5-difluoro-5a-methyl-1,4,4a,5,5a,6-hexahydrocyclopropa[f]indazole-3-carboxamide (Compound 12).

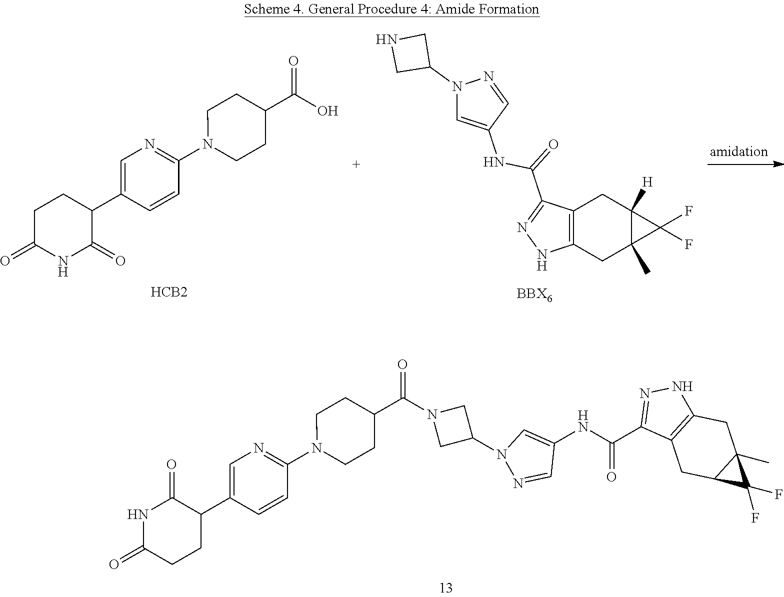

### Scheme 4: Synthesis of Compound 30 Via Amide Formation

A mixture of amine (18 mg, 0.05 mmol), carboxylic acid (16 mg, 0.05 mmol), HATU (28 mg, 0.06 mmol), and i-Pr₂NEt (45 μL, 0.26 mmol) in DMF (200 μL) was allowed to stir at room temperature for one hour. The mixture was purified by HPLC (H₂O:MeCN with 0.1% TFA) to afford the amide product (17 mg, 0.03 mmol, 52%). An exemplary amide coupling is provided in Scheme 4 above where (4aS,5aR)—N-(1-(azetidin-3-yl)-1H-pyrazol-4-yl)-5,5-difluoro-5a-methyl-1,4,4a,5,5a,6-hexahydrocyclopropa[f]indazole-3-carboxamide (BBX₆) was treated with 1-(5-(2,6-dioxopiperidin-3-yl)pyridin-2-yl)piperidine-4-carboxylic acid (HCB2) as described above to provide (4aS,5aR)—N-(1-(1-(1-(5-(2,6-dioxopiperidin-3-yl)pyridin-2-yl)piperidine-4-carbonyl)azetidin-3-yl)-1H-pyrazol-4-yl)-5,5-difluoro-5a-methyl-1,4,4a,5,5a,6-hexahydrocyclopropa[f]indazole-3-carboxamide (Compound 13). ¹H NMR (500 MHz, (CD₃)₂SO) δ 10.93 (s, 1H), 10.23 (s, 1H), 8.18 (s, 1H), 7.88 (d, J=1.9 Hz, 1H), 7.86 (s, 1H), 7.77 (s, 1H), 7.36 (s, 1H), 5.25 (td, J=8.0, 4.0 Hz, 1H), 4.67 (t, J=8.5 Hz, 1H), 4.49 (dd, J=9.0, 5.2 Hz, 1H), 4.29 (t, J=9.1 Hz, 1H), 4.19 (d, J=13.4 Hz, 2H), 4.09 (dd, J=10.1, 5.3 Hz, 1H), 3.92 (dd, J=12.8, 4.8 Hz, 1H), 3.27-3.15 (m, 2H), 3.13-3.00 (m, 3H), 2.82 (dd, J=16.9, 3.3 Hz, 1H), 2.69 (ddt, J=17.7, 12.9, 5.2 Hz, 2H), 2.57 (dt, J=20.7, 5.5 Hz, 1H), 2.29 (qd, J=13.0, 4.4 Hz, 1H), 1.98 (ddq, J=10.4, 5.3, 2.9 Hz, 1H), 1.87-1.75 (m, 3H), 1.62 (d, J=12.8 Hz, 2H), 1.35 (s, 3H). LCMS: C₃₂H₃₅F₂N₉O₄ requires: 647, found: m/z=648 [M+H]⁺. HATU or BOP was typically used as a coupling reagent, but other coupling agents have been contemplated as would be appreciated by a person having skill in the art.

Other amide containing compounds of this disclosure that were synthesized using General Procedure 1 are Compounds 14, 21, 22, 23, and 36.

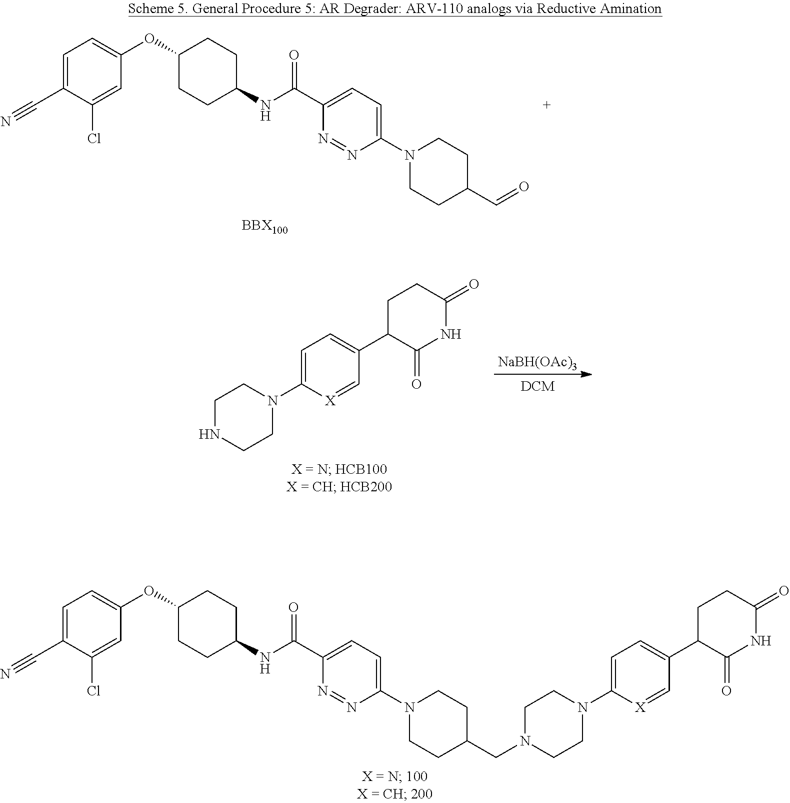

### Scheme 5: Synthesis of Compounds Via Reductive Amination

A mixture of amine (1 eq) and aldehyde (1 eq), in DCM (0.04 M) is stirred at rt for 10 min. NaBH(OAc)₃ (3 eq) would then be added, and the reaction mixture is stirred at rt. The reaction is quenched with H₂O. The reaction mixture is extracted with EtOAc, washed with saturated aqueous NaCl, concentrated, and purified by HPLC (H₂O/MeCN with 0.1% TFA) to afford the amine product. An exemplary reductive amination is provided in Scheme 5 where 3-(6-(piperazin-1-yl)pyridin-3-yl)piperidine-2,6-dione (HCB100) (X═N), or 3-(4-(piperazin-1-yl)phenyl)piperidine-2,6-dione (HCB200) (X═CH) and N-((1r,4r)-4-(3-chloro-4-cyanophenoxy)cyclohexyl)-6-(4-formylpiperidin-1-yl)pyridazine-3-carboxamide (BBX₁₀₀) were reacted as described above to provide N-((1r,4r)-4-(3-chloro-4-cyanophenoxy)cyclohexyl)-6-(4-((4-(5-(2,6-dioxopiperidin-3-yl)pyridin-2-yl)piperazin-1-yl)methyl)piperidin-1-yl)pyridazine-3-carboxamide (100) (X═N) or N-((1r,4r)-4-(3-chloro-4-cyanophenoxy)cyclohexyl)-6-(4-((4-(4-(2,6-dioxopiperidin-3-yl)phenyl)piperazin-1-yl)methyl)piperidin-1-yl)pyridazine-3-carboxamide (200) (X═CH).

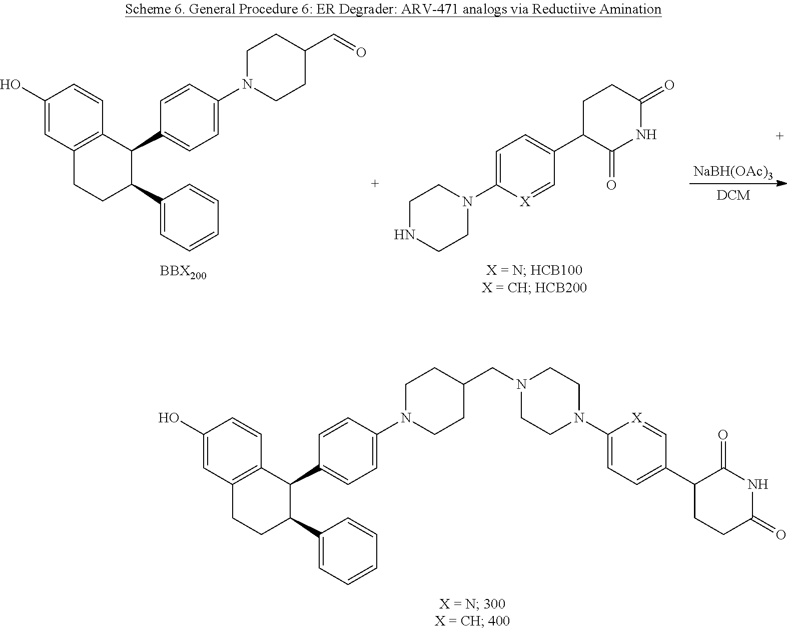

### Scheme 6: Synthesis of Compounds Via Reductive Amination

A mixture of amine (1 eq) and aldehyde (1 eq), in DCM (0.04 M) is stirred at rt for 10 min. NaBH(OAc)₃ (3 eq) is added, and the reaction mixture would be stirred at rt. The reaction is quenched with H₂O. The reaction mixture is extracted with EtOAc, washed with saturated aqueous NaCl, concentrated, and purified by HPLC (H₂O/MeCN with 0.1% TFA) to afford the amine product. An exemplary reductive amination is provided in Scheme 6 where 3-(6-(piperazin-1-yl)pyridin-3-yl)piperidine-2,6-dione (HCB100) (X═N) or 3-(4-(piperazin-1-yl)phenyl)piperidine-2,6-dione (HCB200) (X═CH) and 1-(4-((1R,2S)-6-hydroxy-2-phenyl-1,2,3,4-tetrahydronaphthalen-1-yl)phenyl)piperidine-4-carbaldehyde (BBX₂₀₀) were reacted as described above to provide 3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)pyridin-3-yl)piperidine-2,6-dione (300) (X═N) or 3-(4-(4-((1-(4-((1R,2S)-6-hydroxy-2-phenyl-1,2,3,4-tetrahydronaphthalen-1-yl)phenyl)piperidin-4-yl)methyl)piperazin-1-yl)phenyl)piperidine-2,6-dione (400) (X═CH).

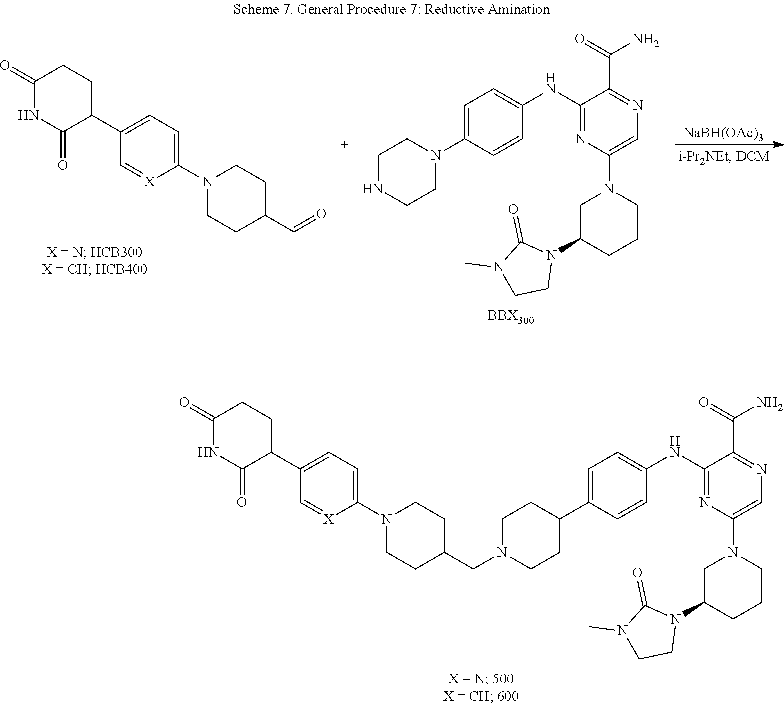

### Scheme 7: Synthesis of Compounds Via Reductive Amination

NaBH(OAc)₃ (21 mg, 0.1 mmol) was added to a solution of amine (BBX₃₀₀) (16 mg, 0.03 mmol), aldehyde (HCB300) (9.9 mg, 0.03 mmol, X═N) and i-Pr₂NEt (70 μL, 0.4 mmol) in DCM (1 mL). The reaction mixture was allowed to stir at rt for 16 h. The reaction was quenched with H₂O. The reaction mixture was extracted with DCM, concentrated, and purified by preparative TLC (10% MeOH/DCM) to afford the amine product (500) (12 mg, 0.01 mmol, 43%, X═N). An exemplary reductive amination is provided in Scheme 7 where 3-(6-(piperazin-1-yl)pyridin-3-yl)piperidine-2,6-dione (HCB300) (X═N) or 3-(4-(piperazin-1-yl)phenyl)piperidine-2,6-dione (HCB400) (X═CH) and (R)-5-(3-(3-methyl-2-oxoimidazolidin-1-yl)piperidin-1-yl)-3-((4-(piperidin-4-yl)phenyl)amino)pyrazine-2-carboxamide (BBX₅₀₀) were reacted as described above to provide 3-((4-(1-((1-(5-(2,6-dioxopiperidin-3-yl)pyridin-2-yl)piperidin-4-yl)methyl)piperidin-4-yl)phenyl)amino)-5-((R)-3-(3-methyl-2-oxoimidazolidin-1-yl)piperidin-1-yl)pyrazine-2-carboxamide (500) (X═N) or 3-((4-(1-((1-(4-(2,6-dioxopiperidin-3-yl)phenyl)piperidin-4-yl)methyl)piperidin-4-yl)phenyl)amino)-5-((R)-3-(3-methyl-2-oxoimidazolidin-1-yl)piperidin-1-yl)pyrazine-2-carboxamide (600) (X═CH).

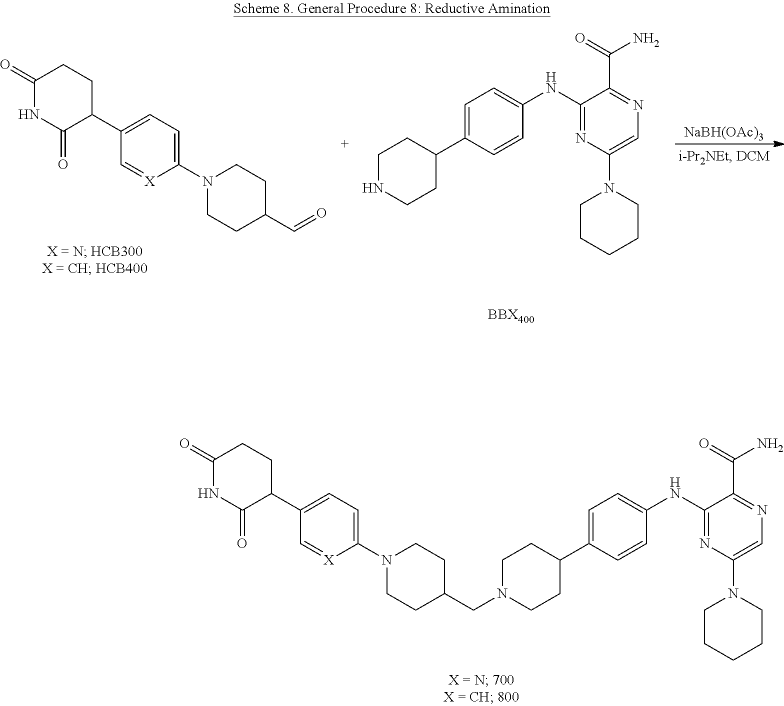

### Scheme 8: Synthesis of Compounds Via Reductive Amination

NaBH(OAc)₃ (19 mg, 0.09 mmol) was added to a solution of amine (BBX₄₀₀) (11 mg, 0.03 mmol), aldehyde (HCB300) (9 mg, 0.03 mmol, X═N) and i-Pr₂NEt (60 μL, 0.36 mmol) in DCM (1 mL). The reaction mixture was allowed to stir at rt for 1 h. The reaction was quenched with H₂O. The reaction mixture was extracted with DCM, concentrated, and purified by preparative TLC (10% MeOH/DCM) to afford the amine product (700) (4.8 mg, 0.01 mmol, 23%, X═N). An exemplary reductive amination is provided in Scheme 8 where 3-(6-(piperazin-1-yl)pyridin-3-yl)piperidine-2,6-dione (HCB300) (X═N) or 3-(4-(piperazin-1-yl)phenyl)piperidine-2,6-dione (HCB400) (X═CH) and 5-(piperidin-1-yl)-3-((4-(piperidin-4-yl)phenyl)amino)pyrazine-2-carboxamide (BBX400) were reacted as described above to provide 3-((4-(1-((1-(5-(2,6-dioxopiperidin-3-yl)pyridin-2-yl)piperidin-4-yl)methyl)piperidin-4-yl)phenyl)amino)-5-(piperidin-1-yl)pyrazine-2-carboxamide (700) (X═N) or 3-((4-(1-((1-(4-(2,6-dioxopiperidin-3-yl)phenyl)piperidin-4-yl)methyl)piperidin-4-yl)phenyl)amino)-5-(piperidin-1-yl)pyrazine-2-carboxamide (800) (X═CH).

### Example 36. 3-(6-(4-(((1-(2-(6,6-dimethyl-4,5,6,7-tetrahydro-1H-indazol-3-yl)-1H-indole-6-carbonyl)piperidin-4-yl)methyl)(methyl)amino)piperidin-1-yl)pyridin-3-yl)piperidine-2,6-dione (1)

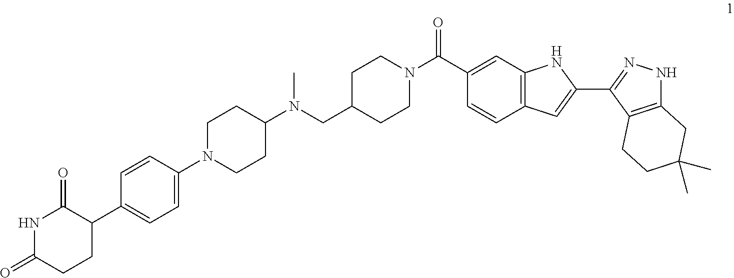 [SMILES: CN(CC1CCN(C(=O)c2ccc3cc(-c4n[nH]c5c4CCC(C)(C)C5)[nH]c3c2)CC1)C1CCN(c2ccc(C3CCC(=O)NC3=O)cc2)CC1]

A mixture of 3-(6-(4-(methyl(piperidin-4-ylmethyl)amino)piperidin-1-yl)pyridin-3-yl)piperidine-2,6-dione (78.0 mg, 0.195 mmol), DMF (0.80 mL), and N,N-diisopropylethylamine (170 μL, 0.130 g, 0.976 mmol) was added to a mixture of 2-(6,6-dimethyl-4,5,6,7-tetrahydro-1H-indazol-3-yl)-1H-indole-6-carboxylic acid (60.4 mg, 0.195 mmol), DMF (0.80 mL), N,N-diisopropylethylamine (170 μL, 0.130 g, 0.976 mmol), and HATU (81.6 mg, 0.215 mmol) and the reaction was allowed to stir at rt for thirty minutes. The reaction was concentrated under reduced pressure and then purified by RP-HPLC (10-80% MeCN in water with 0.1% TFA). The desired freebase was extracted from fractions using DCM and saturated Na₂CO₃. The organic layer was then concentrated under reduced pressure and lyophilized from MeCN:water to give the desired product (0.017 g, mmol, 12.6%). ¹H NMR (500 MHz, (CD₃)₂SO) δ 12.57 (s, 1H), 11.43 (s, 1H), 10.81 (s, 1H), 7.94 (s, 1H), 7.53 (d, J=8.0 Hz, 1H), 7.43 (s, 1H), 7.37 (d, J=2.5 Hz, 1H), 6.98 (d, J=8.1 Hz, 1H), 6.81 (d, J=8.9 Hz, 1H), 6.61 (s, 1H), 4.33 (d, J=12.8 Hz, 2H), 3.73 (dd, J=12.1, 4.9 Hz, 1H), 2.75 (t, J=12.5 Hz, 3H), 2.71-2.65 (m, 3H), 2.43 (s, 2H), 2.27 (d, J=6.5 Hz, 2H), 2.19 (s, 3H), 2.18-2.13 (m, 1H), 2.02-1.95 (m, 1H), 1.73 (d, J=11.8 Hz, 4H), 1.59 (t, J=6.4 Hz, 2H), 1.43-1.33 (m, 2H), 1.11-1.04 (m, 2H), 1.02 (s, 6H). LCMS: C₄₀H₅₀N₈O₃ requires: 690.4, found: m/z=691.7 [M+H]⁺.

### Example 37. 3-(6-((1-((1-(2-(6,6-dimethyl-4,5,6,7-tetrahydro-1H-indazol-3-yl)-1H-indole-6-carbonyl)piperidin-4-yl)methyl)piperidin-4-yl)(methyl)amino)pyridin-3-yl)piperidine-2,6-dione (2)

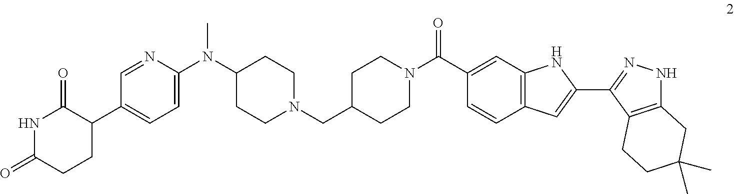 [SMILES: CN(c1ccc(C2CCC(=O)NC2=O)cn1)C1CCN(CC2CCN(C(=O)c3ccc4cc(-c5n[nH]c6c5CCC(C)(C)C6)[nH]c4c3)CC2)CC1]

A mixture of 3-(6-(methyl(1-(piperidin-4-ylmethyl)piperidin-4-yl)amino)pyridin-3-yl)piperidine-2,6-dione (20 mg, 0.05 mmol), DMF (0.5 mL) was added to a mixture of 2-(6,6-dimethyl-4,5,6,7-tetrahydro-1H-indazol-3-yl)-1H-indole-6-carboxylic acid (15 mg, 0.05 mmol), DMF (0.5 mL), N,N-diisopropylethylamine (8.7 μL, 0.05 mmol), and HATU (21 mg, 0.06 mmol) and the reaction was allowed to stir at rt for thirty minutes. The reaction was purified by RP-HPLC (10-80% MeCN in water with 0.1% TFA). The desired freebase was extracted from fractions using DCM and saturated Na₂CO₃. The organic layer was then concentrated under reduced pressure and lyophilized from MeCN:water to give the desired product (0.01 g, 0.01 mmol, 29%). ¹H NMR (500 MHz, (CD₃)₂SO) δ 12.57 (s, 1H), 11.43 (s, 1H), 10.81 (s, 1H), 7.94 (d, J=2.5 Hz, 1H), 7.52 (d, J=8.1 Hz, 1H), 7.43 (s, 1H), 7.37 (dd, J=8.8, 2.5 Hz, 1H), 6.98 (d, J=8.0 Hz, 1H), 6.81 (d, J=8.8 Hz, 1H), 6.61 (s, 1H), 4.33 (d, J=13.1 Hz, 2H), 3.73 (dd, J=12.1, 4.9 Hz, 1H), 2.98-2.82 (m, 2H), 2.79-2.65 (m, 6H), 2.43 (s, 2H), 2.27 (d, J=6.5 Hz, 2H), 2.19 (s, 3H), 2.02-1.95 (m, 1H), 1.76-1.70 (m, 4H), 1.59 (t, J=6.4 Hz, 2H), 1.37 (dd, J=14.1, 10.2 Hz, 2H), 1.25 (s, 1H), 1.06 (s, 2H), 1.02 (s, 6H). LCMS: C₄₀H₅₀N₈O₃ requires: 690.9, found: m/z=691.8 [M+H]⁺.

### Example 38. 3-(6-(6-((1-(2-(6,6-dimethyl-4,5,6,7-tetrahydro-1H-indazol-3-yl)-1H-indole-6-carbonyl)piperidin-4-yl)methyl)-2,6-diazaspiro[3.4]octan-2-yl)pyridin-3-yl)piperidine-2,6-dione (3)

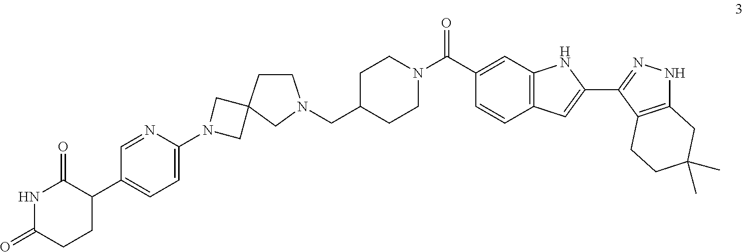 [SMILES: CC1(C)CCc2c(-c3cc4ccc(C(=O)N5CCC(CN6CCC7(C6)CN(c6ccc(C8CCC(=O)NC8=O)cn6)C7)CC5)cc4[nH]3)n[nH]c2C1]

A mixture of 2-(6,6-dimethyl-4,5,6,7-tetrahydro-1H-indazol-3-yl)-1H-indole-6-carboxylic acid (45 mg, 0.15 mmol), DMF (0.99 mL), N,N-diisopropylethylamine (127 μL, 0.09 g, 0.73 mmol), and HATU (61 mg, 0.16 mmol) was treated with a solution of 3-(6-(6-(piperidin-4-ylmethyl)-2,6-diazaspiro[3.4]octan-2-yl)pyridin-3-yl)piperidine-2,6-dione (58 mg, 0.15 mmol) and N,N-diisopropylethylamine (127 μL, 0.09 g, 0.73 mmol) in DMF (0.99 mL). After thirty minutes, the reaction was concentrated under reduced pressure and the residue was purified by RP-HPLC (10-95% MeCN in water) to give the desired product (0.035 g, 35%). ¹H NMR (500 MHz, (CD₃)₂SO) δ 12.57 (s, 1H), 11.43 (s, 1H), 10.80 (s, 1H), 7.91 (s, 1H), 7.53 (d, J=1.9 Hz, 1H), 7.44 (s, 1H), 7.38 (d, J=8.0 Hz, 1H), 7.00 (d, 1H), 6.62 (s, 1H), 6.37 (d, J=8.5 Hz, 1H), 3.87-3.81 (m, 3H), 3.73 (dd, J=12.6, 4.7 Hz, 1H), 3.07-2.79 (m, 2H), 2.78-2.62 (m, 5H), 2.45-2.40 (m, 3H), 2.30 (s, 1H), 2.20-2.12 (m, 1H), 2.09-1.91 (m, 3H), 1.77 (s, 3H), 1.62-1.56 (m, 2H), 1.25 (s, 1H), 1.16-1.05 (m, 2H), 1.02 (s, 6H). LCMS: C₄₀H₄₈N₈O₃ requires: 688.4, found: m/z=689.8 [M+H]⁺.

### Example 39. N-(2-((4aS,5aR)-5,5-difluoro-5a-methyl-1,4,4a,5,5a,6-hexahydrocyclopropa[f]indazol-3-yl)-1H-indol-6-yl)-3-(1-(5-(2,6-dioxopiperidin-3-yl)pyridin-2-yl)piperidin-4-yl)-N-methylpropanamide (4)

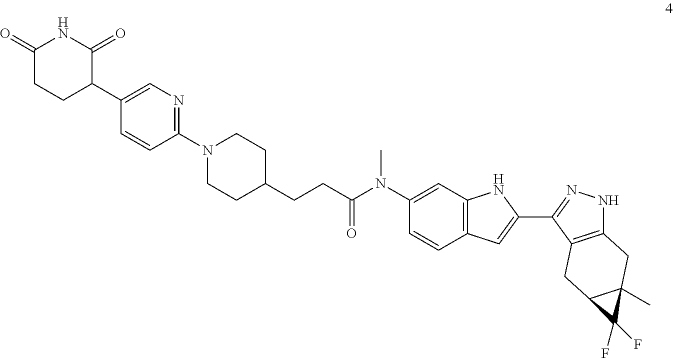 [SMILES: CN(C(=O)CCC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1]

N-(2-((4aS,5aR)-5,5-difluoro-5a-methyl-1,4,4a,5,5a,6-hexahydrocyclopropa[f]indazol-3-yl)-1H-indol-6-yl)-3-(1-(5-(2,6-dioxopiperidin-3-yl)pyridin-2-yl)piperidin-4-yl)-N-methylpropanamide (0.0237 g, 68.4%) was prepared by procedures analogous to Example 62 starting from tert-butyl 3-(piperidin-4-yl)propanoate (400 mg, 1.88 mmol) and 5-bromo-2-fluoropyridine (0.330 g, 1.88 mmol). ¹H NMR (500 MHz, DMSO-d₆) δ 12.70 (s, 1H), 11.43 (s, 1H), 10.89 (s, 1H), 7.84 (s, 1H), 7.68 (s, 1H), 7.58 (d, J=8.2 Hz, 1H), 7.26 (s, 1H), 7.13 (s, 1H), 6.90 (d, J=8.3 Hz, 1H), 6.64 (s, 1H), 4.10 (d, J=13.1 Hz, 2H), 3.84 (d, J=12.5 Hz, 1H), 3.20 (s, 3H), 3.18-2.99 (m, 3H), 2.98-2.80 (m, 3H), 2.75-2.46 (m, 2H), 2.27-2.20 (m, 1H), 2.08 (t, J=7.2 Hz, 2H), 1.98-1.92 (m, 1H), 1.87 (dd, J=15.4, 7.0 Hz, 1H), 1.57 (d, J=13.0 Hz, 2H), 1.49-1.41 (m, 3H), 1.40 (s, 3H), 1.04-0.98 (m, 2H). LCMS: C₃₆H₃₉F₂N₇O₃ requires 655, found: m/z=656 [M+H]⁺.

### Example 40. N-(2-((4aS,5aR)-5,5-difluoro-5a-methyl-1,4,4a,5,5a,6-hexahydrocyclopropa[f]indazol-3-yl)-1H-indol-6-yl)-1-((1-(5-(2,6-dioxopiperidin-3-yl)pyridin-2-yl)piperidin-4-yl)methyl)-N-methylpiperidine-4-carboxamide (5)

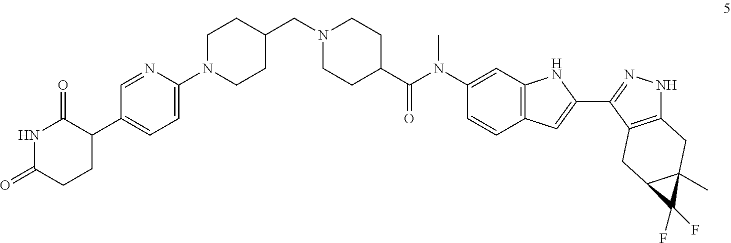 [SMILES: CN(C(=O)C1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1]

A mixture of N-{2-[(4aS,5aR)-5,5-difluoro-5a-methyl-1H,4H,4aH,6H-cyclopropa[f]indazol-3-yl]-1H-indol-6-yl}-N-methylpiperidine-4-carboxamide (22.38 mg, 0.05 mmol) and N,N-diisopropylethylamine (35.00 μL, 0.03 g, 0.20 mmol) in DCM (1.00 mL) was added to rac-1-{5-[(3R)-2,6-dioxopiperidin-3-yl]pyridin-2-yl}piperidine-4-carbaldehyde (15.34 mg, 0.05 mmol) followed by sodium triacetoxyborohydride (21.58 mg, 0.10 mmol). After 1 h, the reaction was quenched with water. The mixture was extracted twice with DCM. The combined organic layers were concentrated and purified by flash chromatography on a 4 g column, eluting by gradient elution with 0 to 20% MeOH/DCM to provide N-{2-[(4aS,5aR)-5,5-difluoro-5a-methyl-1H,4H,4aH,6H-cyclopropa[f]indazol-3-yl]-1H-indol-6-yl}-1-[(1-{5-[(3RS)-2,6-dioxopiperidin-3-yl]pyridin-2-yl}piperidin-4-yl)methyl]-N-methylpiperidine-4-carboxamide (0.0032 g, 6.0%) as a white solid. ¹H NMR (500 MHz, (CD₃)₂SO) δ 12.68 (s, 1H), 11.41 (s, 1H), 10.78 (s, 1H), 7.91 (d, J=2.4 Hz, 1H), 7.58 (d, J=8.5 Hz, 1H), 7.47 (d, J=7.9 Hz, 1H), 7.36-7.31 (m, 1H), 7.25 (s, 1H), 7.11 (d, J=7.8 Hz, 1H), 6.89 (d, J=8.4 Hz, 1H), 6.75 (d, J=8.8 Hz, 1H), 6.63 (s, 1H), 4.21 (d, J=12.7 Hz, 2H), 3.70 (dd, J=12.2, 4.9 Hz, 1H), 3.17 (s, 4H), 3.08-2.97 (m, 2H), 2.84 (d, J=17.2 Hz, 1H), 2.76-2.62 (m, 3H), 2.29 (s, 1H), 2.17 (dd, J=12.7, 8.5 Hz, 1H), 2.01-1.92 (m, 1H), 1.86 (d, J=9.3 Hz, 1H), 1.67 (d, J=12.5 Hz, 5H), 1.55 (br s, 3H), 1.39 (s, 3H), 1.23 (s, 1H), 1.22-1.14 (m, 1H), 1.08-0.95 (m, 2H), 0.90-0.76 (m, 1H). LCMS: C₄₀H₄₆F₂N₈O₃ requires: 724.4, found: m/z=725.6 [M+H]⁺. LCMS: C₄₀H₄₆F₂N₈O₃ requires: 724.4, found: m/z=725.6 [M+H]⁺.

### Example 41. N-(2-((4aS,5aR)-5,5-difluoro-5a-methyl-1,4,4a,5,5a,6-hexahydrocyclopropa[f]indazol-3-yl)-1H-indol-6-yl)-2-(1-((1-(5-(2,6-dioxopiperidin-3-yl)pyridin-2-yl)piperidin-4-yl)methyl)piperidin-4-yl)-N-methylacetamide (6)

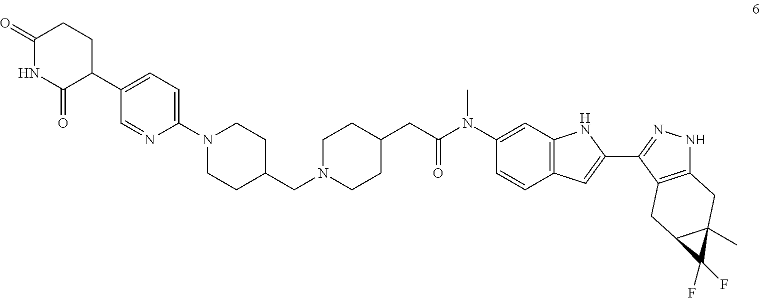 [SMILES: CN(C(=O)CC1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1]

A mixture of N-{2-[(4aS,5aR)-5,5-difluoro-5a-methyl-1H,4H,4aH,6H-cyclopropa[f]indazol-3-yl]-1H-indol-6-yl}-N-methyl-2-(piperidin-4-yl)acetamide (20.50 mg, 0.05 mmol) and N,N-diisopropylethylamine (32.00 μL, 0.02 g, 0.18 mmol) in DCM (1.00 mL) was added to rac-1-{5-[(3R)-2,6-dioxopiperidin-3-yl]pyridin-2-yl}piperidine-4-carbaldehyde (15.50 mg, 0.05 mmol) followed by sodium triacetoxyborohydride (19.16 mg, 0.09 mmol). After 30 min, the reaction was diluted with DCM, concentrated, and purified by flash chromatography on a 4 g column, eluting by gradient elution with 0 to 20% MeOH/DCM to provide N-{2-[(4aS,5aR)-5,5-difluoro-5a-methyl-1H,4H,4aH,6H-cyclopropa[f]indazol-3-yl]-1H-indol-6-yl}-2-{1-[(1-{5-[(3RS)-2,6-dioxopiperidin-3-yl]pyridin-2-yl}piperidin-4-yl)methyl]piperidin-4-yl}-N-methylacetamide (0.017 g, 42.1%) as a white solid. ¹H NMR (500 MHz, (CD₃)₂SO) δ 12.68 (s, 1H), 11.42 (s, 1H), 10.78 (s, 1H), 7.91 (d, J=2.5 Hz, 1H), 7.57 (d, J=8.2 Hz, 1H), 7.33 (dd, J=8.8, 2.5 Hz, 1H), 7.22 (s, 1H), 6.85 (d, J=8.2 Hz, 1H), 6.74 (d, J=8.8 Hz, 1H), 6.63 (s, 1H), 4.20 (d, J=12.7 Hz, 2H), 3.70 (dd, J=12.1, 4.9 Hz, 1H), 3.18 (s, 3H), 3.12 (t, J=8.0 Hz, 1H), 3.08-2.97 (m, 2H), 2.87-2.61 (m, 7H), 2.14 (tt, J=12.5, 6.4 Hz, 3H), 1.96 (dd, J=11.5, 5.8 Hz, 3H), 1.86 (dd, J=14.8, 7.0 Hz, 1H), 1.68 (d, J=12.6 Hz, 5H), 1.56 (d, J=12.5 Hz, 2H), 1.39 (s, 3H), 0.99 (p, J=12.8 Hz, 5H). LCMS: C₄₁H₄₈F₂N₈O₃ requires: 738.4, found: m/z=739.6 [M+H]⁺.

### Example 42. 3-(6-(4-((2-(2-(6,6-dimethyl-4,5,6,7-tetrahydro-1H-indazol-3-yl)-1H-indole-6-carbonyl)-2,6-diazaspiro[3.4]octan-6-yl)methyl)piperidin-1-yl)pyridin-3-yl)piperidine-2,6-dione (7)

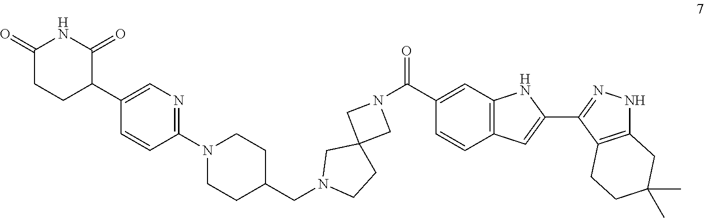 [SMILES: CC1(C)CCc2c(-c3cc4ccc(C(=O)N5CC6(CCN(CC7CCN(c8ccc(C9CCC(=O)NC9=O)cn8)CC7)C6)C5)cc4[nH]3)n[nH]c2C1]

### Step 1: tert-butyl 2-(2-(6,6-dimethyl-4,5,6,7-tetrahydro-1H-indazol-3-yl)-1H-indole-6-carbonyl)-2,6-diazaspiro[3.4]octane-6-carboxylate (7a)

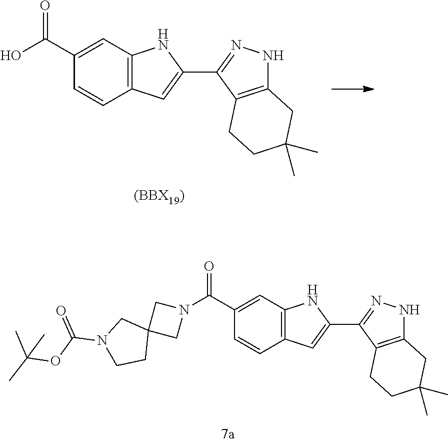

To a mixture of 2-(6,6-dimethyl-1,4,5,7-tetrahydroindazol-3-yl)-1H-indole-6-carboxylic acid (BBX₁₉) (31 mg, 0.10 mmol) and tert-butyl 2,6-diazaspiro[3.4]octane-2-carboxylate (21 mg, 0.10 mmol) in DMF (0.50 mL) was added N,N-diisopropylethylamine (0.05 mL, 0.30 mmol) and (1,2,3-benzotriazol-1-yloxy)tris(dimethylamino)phosphanium; hexafluoro-lambda5-phosphanuide (44 mg, 0.10 mmol). After stirring overnight, 1 mL water was added. The mixture was extracted twice with 1 mL DCM. The combined organic layers were concentrated in vacuo to provide tert-butyl 2-(2-(6,6-dimethyl-4,5,6,7-tetrahydro-1H-indazol-3-yl)-1H-indole-6-carbonyl)-2,6-diazaspiro[3.4]octane-6-carboxylate (7a) (50 mg, 100%). LCMS: C₂₉H₃₇N₅O₃ requires: 503, found: m/z=504 [M+H]⁺.

### Step 2: (2-(6,6-dimethyl-4,5,6,7-tetrahydro-1H-indazol-3-yl)-1H-indol-6-yl)(2,6-diazaspiro[3.4]octan-2-yl)methanone (7b)

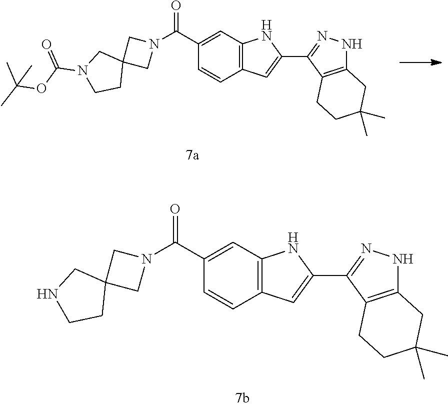 [SMILES: CC1(C)CCc2c(-c3cc4ccc(C(=O)N5CC6(CCN(C(=O)OC(C)(C)C)C6)C5)cc4[nH]3)n[nH]c2C1 ; CC1(C)CCc2c(-c3cc4ccc(C(=O)N5CC6(CCNC6)C5)cc4[nH]3)n[nH]c2C1]

tert-butyl 2-[2-(6,6-dimethyl-1,4,5,7-tetrahydroindazol-3-yl)-1H-indole-6-carbonyl]-2,6-diazaspiro[3.4]octane-6-carboxylate (50 mg, 0.10 mmol) was dissolved in DCM (0.50 mL) and TFA (0.50 mL). After 15 minutes, the mixture was concentrated in vacuo to provide (2-(6,6-dimethyl-4,5,6,7-tetrahydro-1H-indazol-3-yl)-1H-indol-6-yl)(2,6-diazaspiro[3.4]octan-2-yl)methanone (40 mg 100%). LCMS: C₂₄H₂₉N₅O requires: 403, found: m/z=404 [M+H]⁺.

Step 3: 3-(6-(4-((2-(2-(6,6-dimethyl-4,5,6,7-tetrahydro-1H-indazol-3-yl)-1H-indole-6-carbonyl)-2,6-diazaspiro[3.4]octan-6-yl)methyl)piperidin-1-yl)pyridin-3-yl)piperidine-2,6-dione (7)

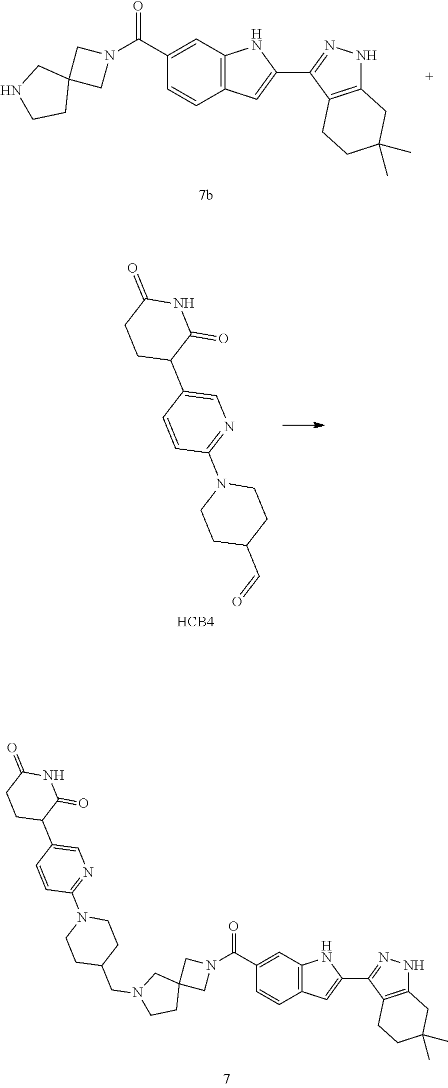 [SMILES: CC1(C)CCc2c(-c3cc4ccc(C(=O)N5CC6(CCNC6)C5)cc4[nH]3)n[nH]c2C1 ; O=CC1CCN(c2ccc(C3CCC(=O)NC3=O)cn2)CC1 ; "HCB4" ; CC1(C)CCc2c(-c3cc4ccc(C(=O)N5CC6(CCN(CC7CCN(c8ccc(C9CCC(=O)NC9=O)cn8)CC7)C6)C5)cc4[nH]3)n[nH]c2C1]

1-(5-(2,6-dioxopiperidin-3-yl)pyridin-2-yl)piperidine-4-carbaldehyde (HCB4) (30 mg, 0.10 mmol) in DMSO (1.50 mL) was added to 3-(6-{2,6-diazaspiro[3.4]octane-2-carbonyl}-1H-indol-2-yl)-6,6-dimethyl-1,4,5,7-tetrahydroindazole (7b) (40 mg, 0.10 mmol) followed by N,N-diisopropylethylamine (0.21 mL, 1.2 mmol). After 10 minutes of stirring vigorously, Borohydride Polymer Supported (200 mg, 0.50 mmol, Sigma Aldrich 2.5 mmol/g loading) was added. After stirring overnight, the mixture was filtered. The resin was washed with an additional 1 mL DMSO. The mixture was concentrated on the Genevac. The crude residue was purified by prep TLC eluted with 15% MeOH/DCM to provide 3-(6-(4-((2-(2-(6,6-dimethyl-4,5,6,7-tetrahydro-1H-indazol-3-yl)-1H-indole-6-carbonyl)-2,6-diazaspiro[3.4]octan-6-yl)methyl)piperidin-1-yl)pyridin-3-yl)piperidine-2,6-dione (0.0076 g, 10.5%) (7). ¹H NMR (500 MHz, DMSO) δ 12.58 (s, 1H), 11.46 (s, 1H), 10.80 (s, 1H), 7.92 (s, 1H), 7.56 (d, J=24.7 Hz, 2H), 7.35 (s, 1H), 7.15 (d, J=8.2 Hz, 1H), 6.75 (s, 1H), 6.63 (s, 1H), 4.32-4.12 (m, 2H), 3.72 (s, 1H), 3.58 (s, 3H), 3.50 (t, J=7.1 Hz, 2H), 3.18-3.11 (m, 2H), 3.08-2.96 (m, 3H), 2.81-2.61 (m, 7H), 2.43 (s, 2H), 2.35-2.12 (m, 3H), 2.04-1.92 (m, 3H), 1.81-1.36 (m, 4H), 1.02 (s, 6H). LCMS: C₄₀H₄₈N₈O₃ requires: 688, found: m/z=689 [M+H]⁺.

### Example 43. 3-(6-(4-((6-(2-(6,6-dimethyl-4,5,6,7-tetrahydro-1H-indazol-3-yl)-1H-indole-6-carbonyl)-2,6-diazaspiro[3.3]heptan-2-yl)methyl)piperidin-1-yl)pyridin-3-yl)piperidine-2,6-dione (8)

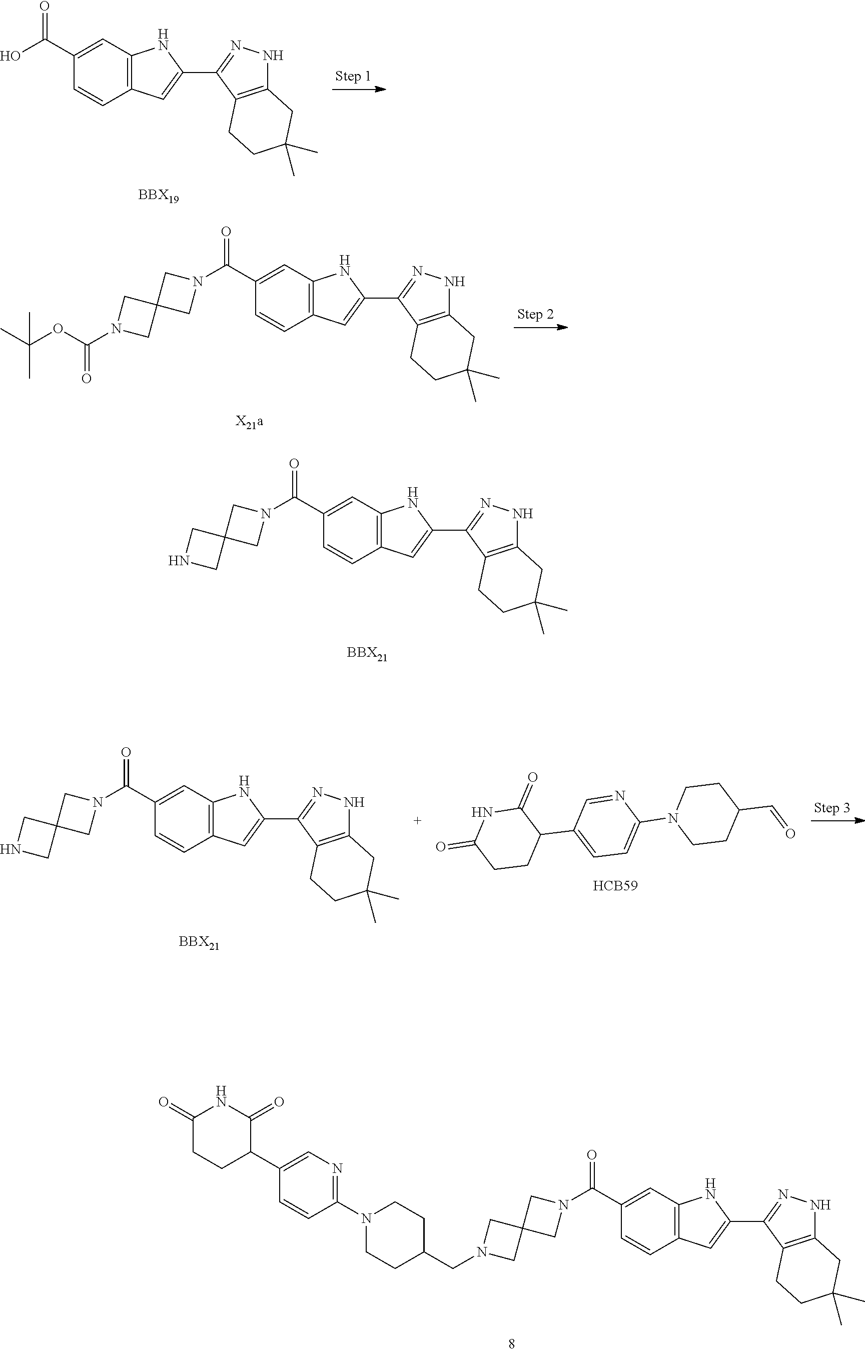

### Step 1: tert-butyl 6-[2-(6,6-dimethyl-1,4,5,7-tetrahydroindazol-3-yl)-1H-indole-6-carbonyl]-2,6-diazaspiro[3.3]heptane-2-carboxylate (X₂₁a)

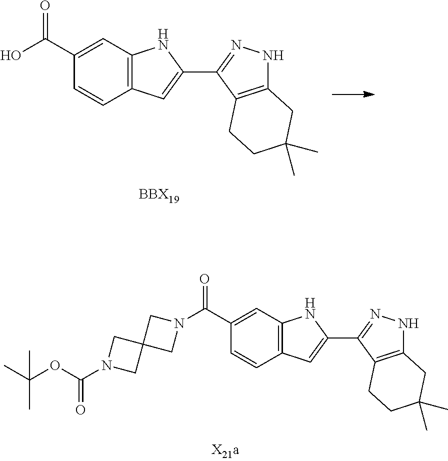

BOP (61.9 mg, 0.14 mmol) was added to a mixture of 2-(6,6-dimethyl-1,4,5,7-tetrahydroindazol-3-yl)-1H-indole-6-carboxylic acid (43.3 mg, 0.14 mmol), tert-butyl 2,6-diazaspiro[3.3]heptane-2-carboxylate (27.8 mg, 0.14 mmol), and N,N-diisopropylethylamine (0.07 mL, 0.05 g, 0.42 mmol) in DMF (0.70 mL). After stirring overnight, water (1 mL) was added. The mixture was extracted twice with DCM (1 mL). The combined organic layers were concentrated to provide tert-butyl 6-[2-(6,6-dimethyl-1,4,5,7-tetrahydroindazol-3-yl)-1H-indole-6-carbonyl]-2,6-diazaspiro[3.3]heptane-2-carboxylate (68 mg, 100%). LCMS: requires: 317, found: m/z=318 [M+H]⁺.

### Step 2: 3-(6-{2,6-diazaspiro[3.3]heptane-2-carbonyl}-1H-indol-2-yl)-6,6-dimethyl-1,4,5,7-tetrahydroindazole (BBX₂₁)

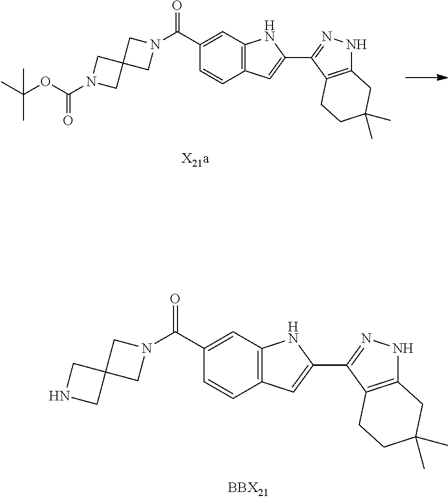 [SMILES: CC1(C)CCc2c(-c3cc4ccc(C(=O)N5CC6(CN(C(=O)OC(C)(C)C)C6)C5)cc4[nH]3)n[nH]c2C1 ; CC1(C)CCc2c(-c3cc4ccc(C(=O)N5CC6(CNC6)C5)cc4[nH]3)n[nH]c2C1]

tert-butyl 6-[2-(6,6-dimethyl-1,4,5,7-tetrahydroindazol-3-yl)-1H-indole-6-carbonyl]-2,6-diazaspiro[3.3]heptane-2-carboxylate (68.6 mg, 0.14 mmol) was stirred in DCM (0.50 mL) and TFA (0.50 mL) for 15 minutes. The mixture was concentrated to provide 3-(6-{2,6-diazaspiro[3.3]heptane-2-carbonyl}-1H-indol-2-yl)-6,6-dimethyl-1,4,5,7-tetrahydroindazole (52 mg, 0.14 mmol). LCMS: C₂₃H₂₇N₅O requires: 389, found: m/z=390 [M+H]⁺.

### Step 3: 3-(6-(4-((6-(2-(6,6-dimethyl-4,5,6,7-tetrahydro-1H-indazol-3-yl)-1H-indole-6-carbonyl)-2,6-diazaspiro[3.3]heptan-2-yl)methyl)piperidin-1-yl)pyridin-3-yl)piperidine-2,6-dione (27)

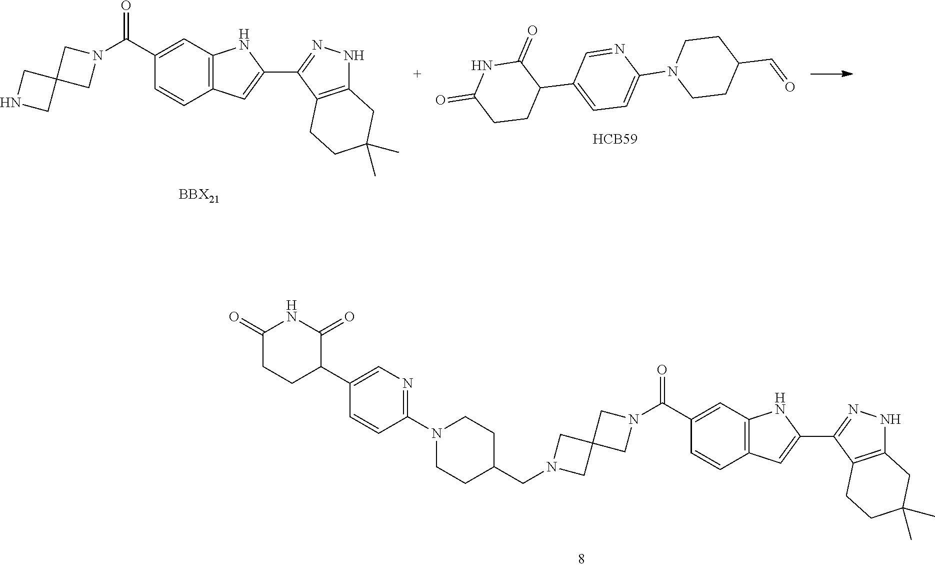 [SMILES: CC1(C)CCc2c(-c3cc4ccc(C(=O)N5CC6(CNC6)C5)cc4[nH]3)n[nH]c2C1 ; O=CC1CCN(c2ccc(C3CCC(=O)NC3=O)cn2)CC1 ; "HCB59" ; CC1(C)CCc2c(-c3cc4ccc(C(=O)N5CC6(CN(CC7CCN(c8ccc(C9CCC(=O)NC9=O)cn8)CC7)C6)C5)cc4[nH]3)n[nH]c2C1]

A mixture of 1-(5-(2,6-dioxopiperidin-3-yl)pyridin-2-yl)piperidine-4-carbaldehyde (42.2 mg, 0.14 mmol) in DMSO (2.00 mL) was added to 3-(6-{2,6-diazaspiro[3.3]heptane-2-carbonyl}-1H-indol-2-yl)-6,6-dimethyl-1,4,5,7-tetrahydroindazole (54.53 mg, 0.14 mmol) followed by N,N-diisopropylethylamine (0.20 mL, 0.14 g, 1.12 mmol). After stirring for five minutes, sodium triacetoxyborohydride (74.2 mg, 0.35 mmol) was added. After stirring overnight, the mixture was quenched with a few drops of water and then concentrated on a Genevac solvent removal system. The crude residue was purified by prep-TLC eluted with 20% MeOH:DCM to provide 3-(6-(4-((6-(2-(6,6-dimethyl-4,5,6,7-tetrahydro-1H-indazol-3-yl)-1H-indole-6-carbonyl)-2,6-diazaspiro[3.3]heptan-2-yl)methyl)piperidin-1-yl)pyridin-3-yl)piperidine-2,6-dione (0.0092 g, 9.6%). ¹H NMR (500 MHz, DMSO-d₆) δ 12.63 (s, 1H), 11.56 (s, 1H), 10.82 (s, 1H), 9.62 (s, 1H), 7.99-7.93 (m, 1H), 7.73 (s, 1H), 7.56 (d, J=8.2 Hz, 1H), 7.50-7.38 (m, 1H), 7.26 (s, 1H), 6.88-6.83 (m, 1H), 6.65 (s, 1H), 4.59 (s, 1H), 4.51-4.36 (m, 3H), 4.34-4.12 (m, 6H), 3.74 (dd, J=12.2, 4.9 Hz, 1H), 3.59 (d, J=7.0 Hz, 1H), 3.11 (s, 2H), 2.81 (t, J=12.2 Hz, 2H), 2.74-2.64 (m, 2H), 2.43 (s, 2H), 2.25-2.09 (m, 1H), 2.01-1.94 (m, 1H), 1.83-1.80 (m, 1H), 1.72-1.66 (m, 2H), 1.60 (t, J=6.4 Hz, 2H), 1.25 (s, 1H), 1.20-1.14 (m, 2H), 1.02 (s, 6H). LCMS: C₃₉H₄₆N₈O₃ requires: 674, found: m/z=675 [M+H]⁺.

### Example 44. 3-(6-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-3-yl)piperidine-2,6-dione (9)

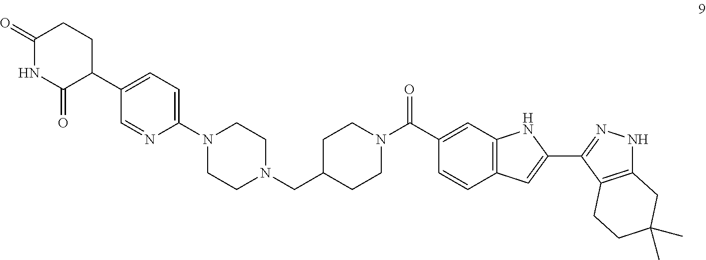 [SMILES: CC1(C)CCc2c(-c3cc4ccc(C(=O)N5CCC(CN6CCN(c7ccc(C8CCC(=O)NC8=O)cn7)CC6)CC5)cc4[nH]3)n[nH]c2C1]

Using general procedure 1, 2-(6,6-dimethyl-4,5,6,7-tetrahydro-1H-indazol-3-yl)-1H-indole-6-carboxylic acid (29 mg, 0.09 mmol) was treated with 3-(6-(4-(piperidin-4-ylmethyl)piperazin-1-yl)pyridin-3-yl)piperidine-2,6-dione (29 mg, 0.08 mmol) to afford the desired product (14 mg, 0.02 mmol, 27%). ¹H NMR (500 MHz, (CD₃)₂SO) δ 11.45 (s, 1H), 10.84 (s, 1H), 9.34 (s, 1H), 8.03 (d, J=2.4 Hz, 1H), 7.56-7.49 (m, 2H), 7.44 (s, 1H), 7.02-6.94 (m, 2H), 6.62 (d, J=2.0 Hz, 1H), 4.35 (d, J=13.7 Hz, 2H), 3.79 (dd, J=12.4, 4.9 Hz, 1H), 3.61 (d, J=11.8 Hz, 2H), 3.21 (t, J=12.7 Hz, 2H), 3.10 (dd, J=18.2, 8.2 Hz, 4H), 2.73-2.62 (m, 3H), 2.54 (s, 1H), 2.50-2.44 (m, 3H), 2.42 (s, 2H), 2.19 (ddd, J=25.6, 13.9, 8.0 Hz, 2H), 2.00-1.93 (m, 1H), 1.77 (d, J=19.2 Hz, 2H), 1.58 (t, J=6.4 Hz, 2H), 1.22 (d, J=13.4 Hz, 3H), 1.01 (s, 6H). LCMS: C₃₈H₄₆F₂N₈O₃ requires: 662, found: m/z=663 [M+H]⁺.

### Example 45. 3-(6-(1′-(2-(6,6-dimethyl-4,5,6,7-tetrahydro-1H-indazol-3-yl)-1H-indole-6-carbonyl)-[4,4′-bipiperidin]-1-yl)pyridin-3-yl)piperidine-2,6-dione (10)

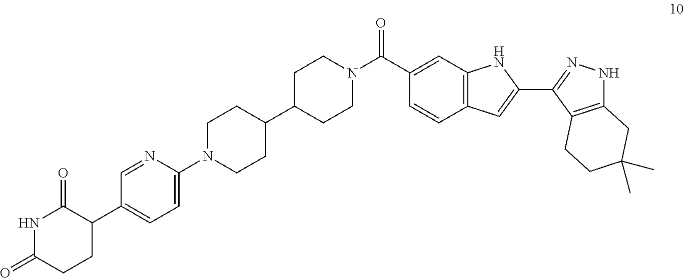 [SMILES: CC1(C)CCc2c(-c3cc4ccc(C(=O)N5CCC(C6CCN(c7ccc(C8CCC(=O)NC8=O)cn7)CC6)CC5)cc4[nH]3)n[nH]c2C1]

Using general procedure 1, 2-(6,6-dimethyl-4,5,6,7-tetrahydro-1H-indazol-3-yl)-1H-indole-6-carboxylic acid (29 mg, 0.09 mmol) was treated with 3-(6-([4,4′-bipiperidin]-1-yl)pyridin-3-yl)piperidine-2,6-dione (28 mg, 0.08 mmol) to afford the desired product (11 mg, 0.02 mmol, 22%). ¹H NMR (500 MHz, (CD₃)₂SO) δ 11.40 (s, 1H), 10.89 (s, 1H), 7.87 (s, 1H), 7.52 (d, J=8.1 Hz, 1H), 7.43 (s, 1H), 6.98 (d, J=8.0 Hz, 1H), 6.61 (s, 1H), 4.24 (d, J=13.0 Hz, 2H), 3.85 (s, 1H), 3.20-3.13 (m, 4H), 2.67 (td, J=14.0, 8.7 Hz, 4H), 2.55 (d, J=14.1 Hz, 1H), 2.42 (s, 2H), 2.25 (d, J=13.3 Hz, 1H), 2.00-1.93 (m, 1H), 1.81 (d, J=11.7 Hz, 2H), 1.72 (s, 1H), 1.57 (p, J=8.0, 7.1 Hz, 7H), 1.41 (s, 1H), 1.36-1.10 (m, 8H), 1.01 (s, 6H), 0.94 (t, J=7.3 Hz, 6H). LCMS: C₃₈H₄₅N₇O₃ requires: 647, found: m/z=648 [M+H]⁺.

### Example 46. 3-(6-(4-(((1-(2-(6,6-dimethyl-4,5,6,7-tetrahydro-1H-indazol-3-yl)-1H-indole-6-carbonyl)piperidin-4-yl)(methyl)amino)methyl)piperidin-1-yl)pyridin-3-yl)piperidine-2,6-dione (11)

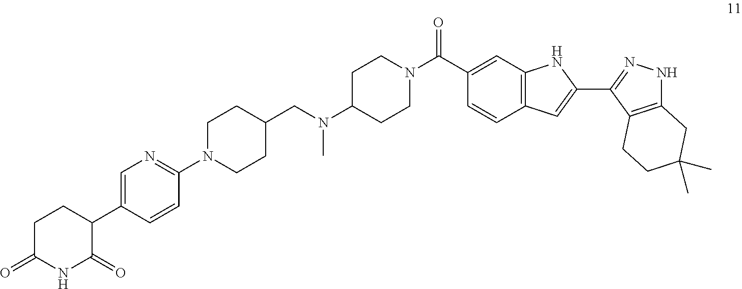 [SMILES: CN(CC1CCN(c2ccc(C3CCC(=O)NC3=O)cn2)CC1)C1CCN(C(=O)c2ccc3cc(-c4n[nH]c5c4CCC(C)(C)C5)[nH]c3c2)CC1]

Using general procedure 3-(6-(4-((methyl(piperidin-4-yl)amino)methyl)piperidin-1-yl)pyridin-3-yl)piperidine-2,6-dione (HCB41) (12 mg, 0.03 mmol) was treated with 1, 2-(6,6-dimethyl-4,5,6,7-tetrahydro-1H-indazol-3-yl)-1H-indole-6-carboxylic acid (BBX₁₉) (9.3 mg, 0.03 mmol) to afford the desired product (2.9 mg, 13%). ¹H NMR (500 MHz, CD₃CN) δ 9.90 (s, 2H), 8.82 (s, 1H), 7.97 (s, 1H), 7.64 (dd, J=14.7, 8.7 Hz, 2H), 7.54 (s, 1H), 7.13 (d, J=8.2 Hz, 1H), 7.04 (d, J=9.3 Hz, 1H), 6.74 (s, 1H), 5.48 (s, 2H), 4.27 (s, 3H), 3.78 (d, J=12.4 Hz, 1H), 3.55 (d, J=12.2 Hz, 1H), 3.22-2.85 (m, 4H), 2.85-2.65 (m, 7H), 1.77 (d, J=11.2 Hz, 1H), 1.67 (t, J=6.4 Hz, 2H), 1.47-1.19 (m, 6H), 1.07 (s, 6H). LCMS: C₄₀H₅₀N₈O₃ requires: 690.4, found: m/z=691.8 [M+H]⁺.

### Example 47. (4aS,5aR)—N-(1-(1-((1-(5-(2,6-dioxopiperidin-3-yl)pyridin-2-yl)piperidin-4-yl)methyl)azetidin-3-yl)-1H-pyrazol-4-yl)-5,5-difluoro-5a-methyl-1,4,4a,5,5a,6-hexahydrocyclopropa[f]indazole-3-carboxamide (12)

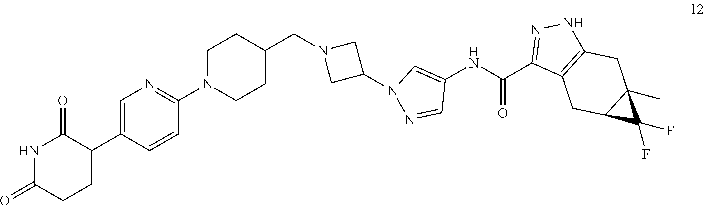 [SMILES: C[C@@]12Cc3[nH]nc(C(=O)Nc4cnn(C5CN(CC6CCN(c7ccc(C8CCC(=O)NC8=O)cn7)CC6)C5)c4)c3C[C@@H]1C2(F)F]

Using general procedure 3, (4aS,5aR)—N-(1-(azetidin-3-yl)-1H-pyrazol-4-yl)-5,5-difluoro-5a-methyl-1,4,4a,5,5a,6-hexahydrocyclopropa[f]indazole-3-carboxamide (20 mg, 0.06 mmol) was treated with 1-(5-(2,6-dioxopiperidin-3-yl)pyridin-2-yl)piperidine-4-carbaldehyde (17 mg, 0.06 mmol) to afford the desired product (23 mg, 0.04 mmol, 64%). ¹H NMR (500 MHz, (CD₃)₂SO) δ 13.05 (s, 1H), 10.91 (s, 1H), 10.29 (d, J=16.5 Hz, 1H), 10.12 (s, 1H), 8.30-8.20 (m, 1H), 7.92 (d, J=2.3 Hz, 1H), 7.85 (s, 1H), 7.76 (s, 1H), 7.25 (s, 1H), 5.38 (q, J=8.1 Hz, 1H), 4.69 (d, J=9.9 Hz, 1H), 4.63-4.56 (m, 1H), 4.51 (d, J=11.1 Hz, 1H), 4.39 (d, J=10.0 Hz, 2H), 4.23 (d, J=13.3 Hz, 2H), 3.88 (dd, J=12.9, 4.8 Hz, 1H), 3.09-3.01 (m, 5H), 2.82 (dd, J=17.1, 3.2 Hz, 1H), 2.69 (ddd, J=17.8, 12.9, 5.4 Hz, 1H), 2.56 (dt, J=17.7, 3.8 Hz, 1H), 2.26 (qd, J=12.9, 4.5 Hz, 1H), 2.02-1.92 (m, 2H), 1.85-1.74 (m, 3H), 1.35 (s, 3H), 1.28 (t, J=11.1 Hz, 2H). LCMS: C₃₂H₃₇F₂N₉O₃ requires: 633, found: m/z=634 [M+H]⁺.

### Example 48. (4aS,5aR)—N-(1-(1-(1-(5-(2,6-dioxopiperidin-3-yl)pyridin-2-yl)piperidine-4-carbonyl)azetidin-3-yl)-1H-pyrazol-4-yl)-5,5-difluoro-5a-methyl-1,4,4a,5,5a,6-hexahydrocyclopropa[f]indazole-3-carboxamide (13)

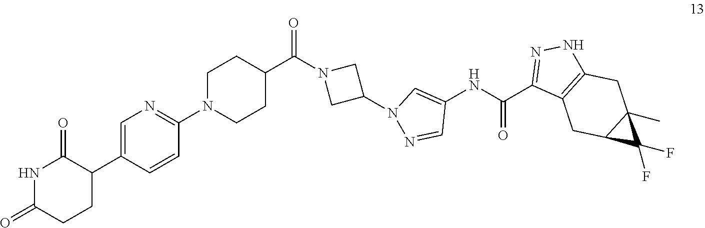 [SMILES: C[C@@]12Cc3[nH]nc(C(=O)Nc4cnn(C5CN(C(=O)C6CCN(c7ccc(C8CCC(=O)NC8=O)cn7)CC6)C5)c4)c3C[C@@H]1C2(F)F]

Using general procedure 4, (4aS,5aR)—N-(1-(azetidin-3-yl)-1H-pyrazol-4-yl)-5,5-difluoro-5a-methyl-1,4,4a,5,5a,6-hexahydrocyclopropa[f]indazole-3-carboxamide (18 mg, 0.05 mmol) was treated with 1-(5-(2,6-dioxopiperidin-3-yl)pyridin-2-yl)piperidine-4-carboxylic acid (16 mg, 0.05 mmol) to afford the desired product (17 mg, 0.03 mmol, 52%). ¹H NMR (500 MHz, (CD₃)₂SO) δ 10.93 (s, 1H), 10.23 (s, 1H), 8.18 (s, 1H), 7.88 (d, J=1.9 Hz, 1H), 7.86 (s, 1H), 7.77 (s, 1H), 7.36 (s, 1H), 5.25 (td, J=8.0, 4.0 Hz, 1H), 4.67 (t, J=8.5 Hz, 1H), 4.49 (dd, J=9.0, 5.2 Hz, 1H), 4.29 (t, J=9.1 Hz, 1H), 4.19 (d, J=13.4 Hz, 2H), 4.09 (dd, J=10.1, 5.3 Hz, 1H), 3.92 (dd, J=12.8, 4.8 Hz, 1H), 3.27-3.15 (m, 2H), 3.13-3.00 (m, 3H), 2.82 (dd, J=16.9, 3.3 Hz, 1H), 2.69 (ddt, J=17.7, 12.9, 5.2 Hz, 2H), 2.57 (dt, J=20.7, 5.5 Hz, 1H), 2.29 (qd, J=13.0, 4.4 Hz, 1H), 1.98 (ddq, J=10.4, 5.3, 2.9 Hz, 1H), 1.87-1.75 (m, 3H), 1.62 (d, J=12.8 Hz, 2H), 1.35 (s, 3H). LCMS: C₃₂H₃₅F₂N₉O₄ requires: 647, found: m/z=648 [M+H]⁺.

### Example 49. (4aS,5aR)—N-(1-(1-(1-(5-(2,6-dioxopiperidin-3-yl)pyridin-2-yl)azetidine-3-carbonyl)azetidin-3-yl)-1H-pyrazol-4-yl)-5,5-difluoro-5a-methyl-1,4,4a,5,5a,6-hexahydrocyclopropa[f]indazole-3-carboxamide (14)

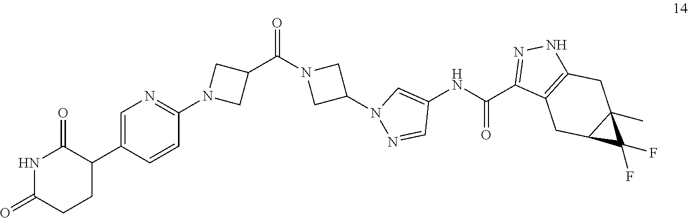 [SMILES: C[C@@]12Cc3[nH]nc(C(=O)Nc4cnn(C5CN(C(=O)C6CN(c7ccc(C8CCC(=O)NC8=O)cn7)C6)C5)c4)c3C[C@@H]1C2(F)F]

Using general procedure 4, (4aS,5aR)—N-(1-(azetidin-3-yl)-1H-pyrazol-4-yl)-5,5-difluoro-5a-methyl-1,4,4a,5,5a,6-hexahydrocyclopropa[f]indazole-3-carboxamide (18 mg, 0.05 mmol) was treated with 1-(5-(2,6-dioxopiperidin-3-yl)pyridin-2-yl)azetidine-3-carboxylic acid (15 mg, 0.05 mmol) to afford the desired product (21 mg, 0.03 mmol, 66%). ¹H NMR (500 MHz, (CD₃)₂SO) δ 12.99 (s, 1H), 10.92 (s, 1H), 10.23 (s, 1H), 8.19 (s, 1H), 7.90 (d, J=2.1 Hz, 1H), 7.84 (s, 1H), 7.77 (s, 1H), 6.84 (s, 1H), 5.27 (ddd, J=13.4, 8.1, 5.3 Hz, 1H), 4.56 (t, J=8.5 Hz, 1H), 4.42-4.32 (m, 4H), 4.24 (s, 2H), 4.15 (dd, J=10.1, 5.3 Hz, 1H), 3.92-3.85 (m, 1H), 3.69 (ddd, J=14.8, 8.7, 6.0 Hz, 1H), 3.12-3.00 (m, 3H), 2.82 (dd, J=17.6, 3.1 Hz, 1H), 2.74-2.62 (m, 1H), 2.61-2.53 (m, 1H), 2.32-2.21 (m, 1H), 2.01-1.94 (m, 1H), 1.78 (d, J=12.7 Hz, 1H), 1.35 (s, 3H). LCMS: C₃₀H₃₁F₂N₉O₄ requires: 619, found: m/z=620 [M+H]⁺.

### Example 50. 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-3-yl)-1-methylpiperidine-2,6-dione (15)

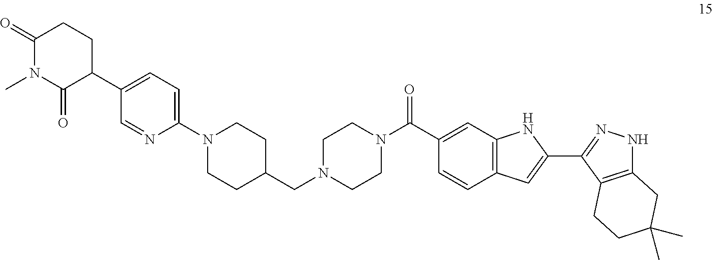 [SMILES: CN1C(=O)CCC(c2ccc(N3CCC(CN4CCN(C(=O)c5ccc6cc(-c7n[nH]c8c7CCC(C)(C)C8)[nH]c6c5)CC4)CC3)nc2)C1=O]

A mixture of 1-(5-(1-methyl-2,6-dioxopiperidin-3-yl)pyridin-2-yl)piperidine-4-carbaldehyde (50 mg, 0.16 mmol), DCM (1.60 mL), WA (1.60 mL), and (2-(6,6-dimethyl-4,5,6,7-tetrahydro-1H-indazol-3-yl)-1H-indol-6-yl)(piperazin-1-yl)methanone (59.85 mg, 0.16 mmol) was treated with sodium triacetoxyborohydride (67.2 mg, 0.32 mmol). After sixty minutes, the reaction was purified by silica gel column chromatography (0-10% MeOH in DCM, step gradient). The desired compound was re-purified by RP-HPLC (10-95% MeCN in water with 0.1% TFA) to provide the desired product (15.2 mg, 14.2%). ¹H NMR (500 MHz, CDCl₃) δ 10.23 (s, 1H), 10.07 (s, 1H), 8.06 (d, J=2.5 Hz, 1H), 7.59 (d, J=8.1 Hz, 1H), 7.51 (s, 1H), 7.31 (dd, J=8.8, 2.5 Hz, 1H), 7.13 (dd, J=8.1, 1.4 Hz, 1H), 6.73-6.70 (m, 1H), 6.67 (d, J=8.9 Hz, 1H), 4.29 (d, J=12.7 Hz, 2H), 3.68 (dd, J=10.4, 5.2 Hz, 1H), 2.89-2.79 (m, 2H), 2.79-2.74 (m, 2H), 2.73-2.65 (m, 1H), 2.44 (s, 2H), 2.25 (qt, J=14.7, 7.7 Hz, 3H), 1.85 (d, J=13.0 Hz, 2H), 1.77 (s, 1H), 1.65 (t, J=6.3 Hz, 2H), 1.24 (d, J=11.9 Hz, 4H), 1.05 (s, 6H). LCMS: C₃₉H₄₈N₈O₃ requires 676.4, found: m/z=677.7 [M+H]⁺.

### Example 51. 3-(6-(4-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decane-8-carbonyl)piperidin-1-yl)pyridin-3-yl)piperidine-2,6-dione (16)

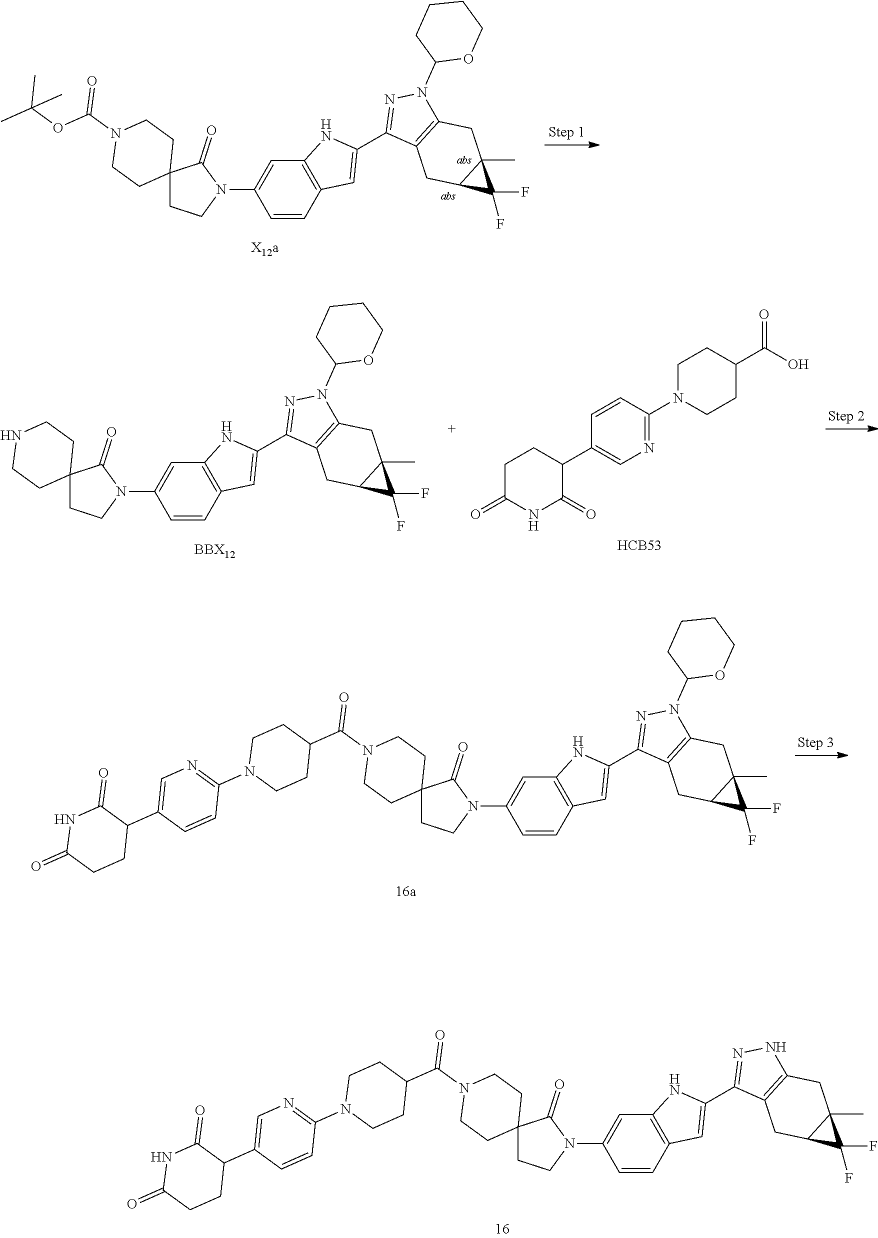 [SMILES: CC(C)(C)OC(=O)N1CCC2(CC1)CCN(c1ccc3cc(-c4nn(C5CCCCO5)c5c4C[C@@H]4C(F)(F)[C@]4(C)C5)[nH]c3c1)C2=O ; C[C@@]12Cc3c(c(-c4cc5ccc(N6CCC7(CCNCC7)C6=O)cc5[nH]4)nn3C3CCCCO3)C[C@@H]1C2(F)F ; C[C@@]12Cc3c(c(-c4cc5ccc(N6CCC7(CCN(C(=O)C8CCN(c9ccc(C%10CCC(=O)NC%10=O)cn9)CC8)CC7)C6=O)cc5[nH]4)nn3C3CCCCO3)C[C@@H]1C2(F)F ; O=C1CCC(c2ccc(N3CCC(C(=O)O)CC3)nc2)C(=O)N1 ; "HCB53" ; C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F]

### Step 1: 2-{2-[(4aS,5aR)-5,5-difluoro-5a-methyl-1-(oxan-2-yl)-4H,4aH,6H-cyclopropa[f]indazol-3-yl]-1H-indol-6-yl}-2,8-diazaspiro[4.5]decan-1-one (BBX₁₂)

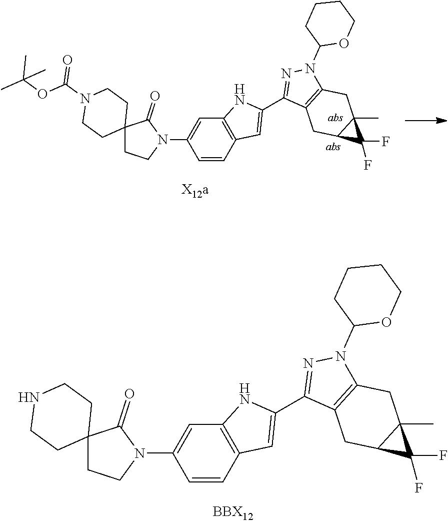 [SMILES: CC(C)(C)OC(=O)N1CCC2(CC1)CCN(c1ccc3cc(-c4nn(C5CCCCO5)c5c4C[C@@H]4C(F)(F)[C@]4(C)C5)[nH]c3c1)C2=O ; C[C@@]12Cc3c(c(-c4cc5ccc(N6CCC7(CCNCC7)C6=O)cc5[nH]4)nn3C3CCCCO3)C[C@@H]1C2(F)F]

tert-butyl 2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carboxylate (31.00 mg, 0.05 mmol) was stirred in DCM (1.50 mL) and TFA (0.50 mL) for ten minutes. The mixture was concentrated in vacuo to provide 2-{2-[(4aS,5aR)-5,5-difluoro-5a-methyl-1-(oxan-2-yl)-4H,4aH,6H-cyclopropa[f]indazol-3-yl]-1H-indol-6-yl}-2,8-diazaspiro[4.5]decan-1-one (26.8 mg, 100%). LCMS: C₃₀H₃₅F₂N₅O requires: 535, found: m/z=536 [M+H]⁺.

### Step 2: (3RS)-3-{6-[4-(2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carbonyl)piperidin-1-yl]pyridin-3-yl}piperidine-2,6-dione (16a)

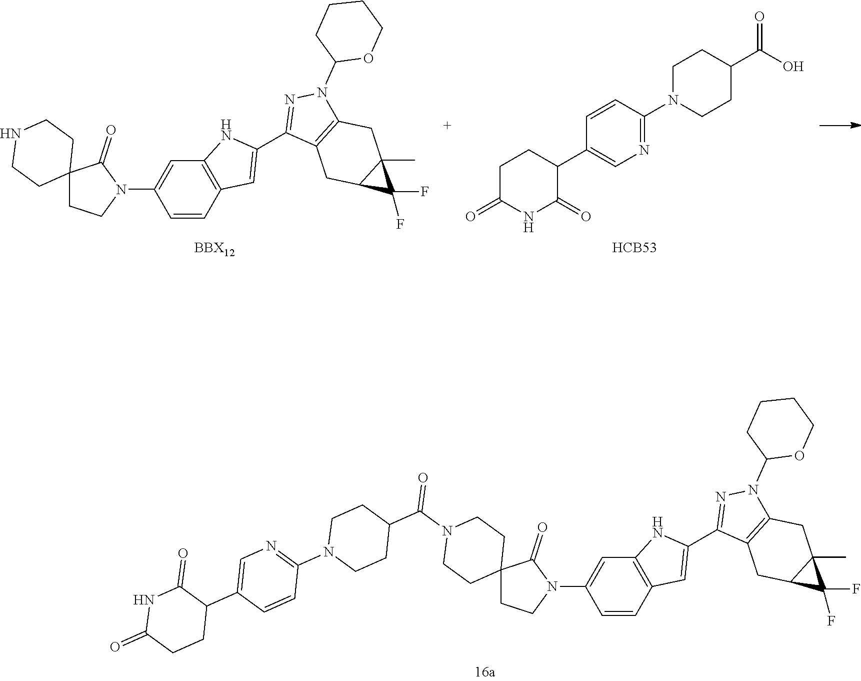 [SMILES: C[C@@]12Cc3c(c(-c4cc5ccc(N6CCC7(CCNCC7)C6=O)cc5[nH]4)nn3C3CCCCO3)C[C@@H]1C2(F)F ; C[C@@]12Cc3c(c(-c4cc5ccc(N6CCC7(CCN(C(=O)C8CCN(c9ccc(C%10CCC(=O)NC%10=O)cn9)CC8)CC7)C6=O)cc5[nH]4)nn3C3CCCCO3)C[C@@H]1C2(F)F ; O=C1CCC(c2ccc(N3CCC(C(=O)O)CC3)nc2)C(=O)N1 ; "HCB53"]

To a mixture of 2-{2-[(4aS,5aR)-5,5-difluoro-5a-methyl-1-(oxan-2-yl)-4H,4aH,6H-cyclopropa[f]indazol-3-yl]-1H-indol-6-yl}-2,8-diazaspiro[4.5]decan-1-one (26.8 mg, 0.05 mmol) in DCM (1.00 mL) was added N,N-diisopropylethylamine (0.05 mL, 0.04 g, 0.30 mmol) followed by 1-(5-(2,6-dioxopiperidin-3-yl)pyridin-2-yl)piperidine-4-carboxylic acid (15.8 mg, 0.05 mmol) then HATU (19.01 mg, 0.05 mmol) and DMF (1.00 mL). After stirring overnight, water was added and the mixture was extracted twice with 10% MeOH:DCM. The combined organic layers were concentrated and the crude residue was purified by preparative TLC eluted with 20% MeOH:DCM to provide (3RS)-3-{6-[4-(2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carbonyl)piperidin-1-yl]pyridin-3-yl}piperidine-2,6-dione (0.0145 g, 34.7%). LCMS: C₄₆H₅₂F₂N₈O₅ requires: 834, found: m/z=835 [M+H]⁺.

### Step 3: 3-(6-(4-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decane-8-carbonyl)piperidin-1-yl)pyridin-3-yl)piperidine-2,6-dione (16)

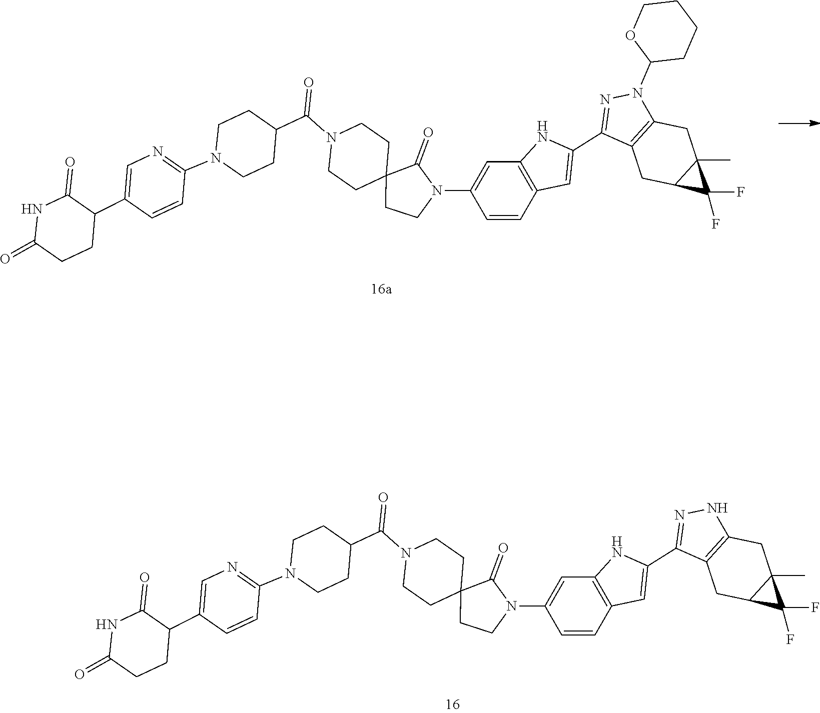 [SMILES: C[C@@]12Cc3c(c(-c4cc5ccc(N6CCC7(CCN(C(=O)C8CCN(c9ccc(C%10CCC(=O)NC%10=O)cn9)CC8)CC7)C6=O)cc5[nH]4)nn3C3CCCCO3)C[C@@H]1C2(F)F ; C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F]

A mixture of (3RS)-3-{6-[4-(2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carbonyl)piperidin-1-yl]pyridin-3-yl}piperidine-2,6-dione (14.5 mg, 0.02 mmol) and para-toluene sulfonate hydrate (6.6 mg, 0.03 mmol) in EtOH (2.00 mL) was heated in a 90° C. heating block for twenty minutes. The mixture was then cooled and dilute aq. sodium bicarbonate was added. The mixture was extracted with DCM then with 10% MeOH:DCM. The combined organic layers were concentrated and the crude residue was purified by preparative TLC eluted with 10% MeOH:DCM to provide 3-(6-(4-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decane-8-carbonyl)piperidin-1-yl)pyridin-3-yl)piperidine-2,6-dione (0.0046 g, 35.3%). ¹H NMR (500 MHz, DMSO-d₆) δ 12.62 (s, 1H), 11.25 (s, 1H), 10.88 (s, 1H), 7.93 (d, J=2.4 Hz, 1H), 7.76 (s, 1H), 7.63 (s, 1H), 7.50 (d, J=8.5 Hz, 1H), 7.26 (d, J=8.6 Hz, 1H), 7.09 (s, 1H), 6.57 (s, 1H), 4.32-4.22 (m, 3H), 4.04 (d, J=13.7 Hz, 1H), 3.88 (t, J=6.9 Hz, 2H), 3.30 (t, J=12.4 Hz, 1H), 3.21-2.95 (m, 5H), 2.94-2.80 (m, 2H), 2.75-2.64 (m, 1H), 2.32-2.08 (m, 3H), 2.03-1.97 (m, 1H), 1.86 (dd, J=14.9, 7.1 Hz, 1H), 1.77-1.74 (m, 3H), 1.66-1.57 (m, 5H), 1.40 (s, 3H), 1.34-1.20 (m, 2H), 0.95-0.83 (m, 1H). LCMS: C₄₁H₄₄F₂N₈O₄ requires: 750, found: m/z=751 [M+H]⁺.

### Example 52. 3-(6-(4-(2-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)ethyl)piperidin-1-yl)pyridin-3-yl)piperidine-2,6-dione (17)

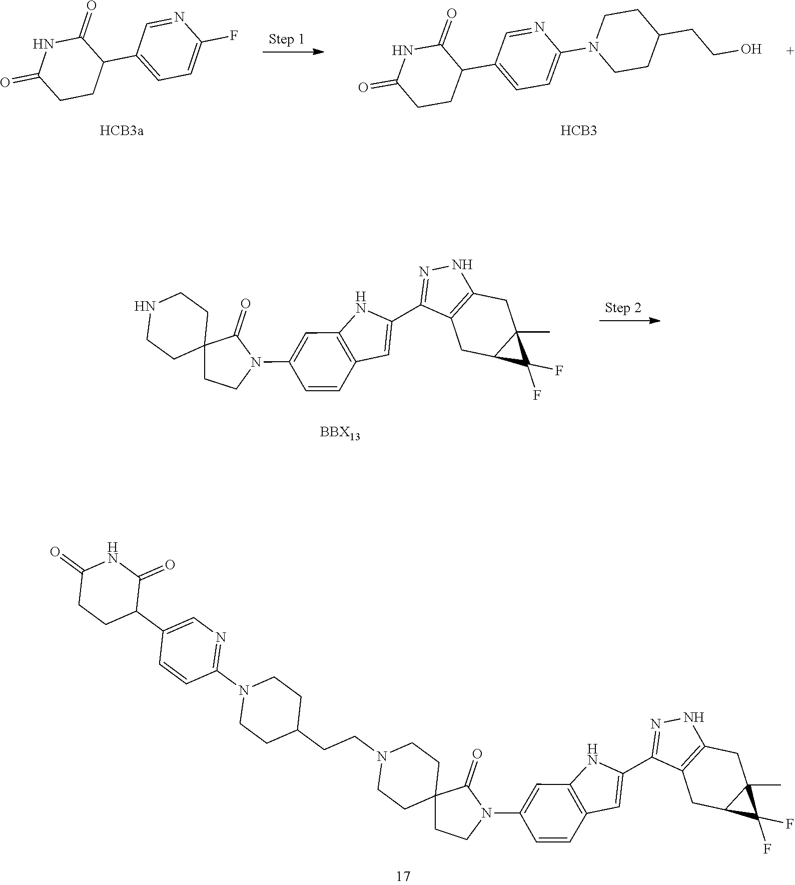

### Step 1: 3-(6-(4-(2-hydroxyethyl)piperidin-1-yl)pyridin-3-yl)piperidine-2,6-dione (HCB3)

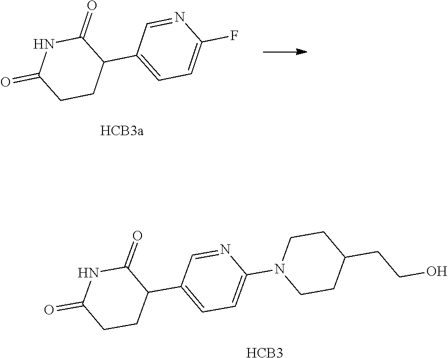

A mixture of r3-(6-fluoropyridin-3-yl)piperidine-2,6-dione (43.4 mg, 0.21 mmol), 4-piperidineethanol (26.9 mg, 0.21 mmol), and N,N-diisopropylethylamine (0.07 mL, 0.05 g, 0.42 mmol) in DMSO (1.00 mL) was heated in a 100° C. heating block for two days. Water was added and the mixture was extracted three times with ethyl acetate. The combined organic layers were concentrated and the crude residue was purified by preparative TLC in 5% MeOH:DCM to provide 3-(6-(4-(2-hydroxyethyl)piperidin-1-yl)pyridin-3-yl)piperidine-2,6-dione (0.0149 g, 22.5%). LCMS: C₁₇H₂₃N₃O₃ requires: 317, found: m/z=318 [M+H]⁺.

### Step 2: 3-(6-(4-(2-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)ethyl)piperidin-1-yl)pyridin-3-yl)piperidine-2,6-dione (17)

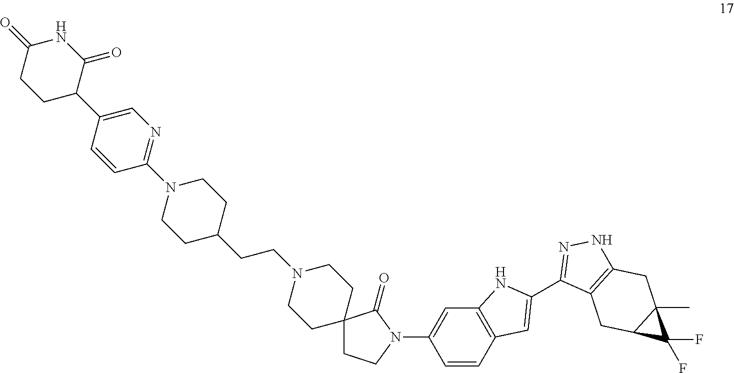 [SMILES: C[C@@]12Cc3[nH]nc(-c4cc5ccc(N6CCC7(CCN(CCC8CCN(c9ccc(C%10CCC(=O)NC%10=O)cn9)CC8)CC7)C6=O)cc5[nH]4)c3C[C@@H]1C2(F)F]

3-(6-(4-(2-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)ethyl)piperidin-1-yl)pyridin-3-yl)piperidine-2,6-dione (0.0126 g, 93.7%) was prepared by procedures analogous to Example 67 starting from 3-(6-(4-(2-hydroxyethyl)piperidin-1-yl)pyridin-3-yl)piperidine-2,6-dione (14.9 mg, 0.05 mmol) and 2-{2-[(4aS,5aR)-5,5-difluoro-5a-methyl-1H,4H,4aH,6H-cyclopropa[f]indazol-3-yl]-1H-indol-6-yl}-2,8-diazaspiro[4.5]decan-1-one (22.6 mg, 0.05 mmol). ¹H NMR (500 MHz, DMSO-d₆) δ 12.64 (s, 1H), 11.31 (s, 1H), 10.82 (s, 1H), 9.13 (s, 1H), 7.95 (d, J=2.5 Hz, 1H), 7.74 (s, 1H), 7.51 (d, J=8.4 Hz, 1H), 7.41-7.35 (m, 1H), 7.25 (d, J=8.6 Hz, 1H), 6.82 (d, J=8.8 Hz, 1H), 6.58-6.52 (m, 1H), 4.29 (d, J=12.8 Hz, 2H), 3.88 (s, 2H), 3.74 (dd, J=12.3, 4.9 Hz, 1H), 3.60-3.56 (m, 1H), 3.30 (s, 2H), 3.23-2.93 (m, 4H), 2.90-2.61 (m, 4H), 2.20-2.16 (m, 2H), 2.10-1.93 (m, 2H), 1.87-1.83 (m, 3H), 1.80-1.72 (m, 2H), 1.70-1.52 (m, 4H), 1.40 (s, 3H), 1.33-1.10 (m, 4H). LCMS: C₄₂H₄₈F₂N₈O₃ requires: 750, found: m/z=751 [M+H]⁺.

### Example 53. 2-(6,6-dimethyl-4,5,6,7-tetrahydro-1H-indazol-3-yl)-N-(1-((1-(5-(2,6-dioxopiperidin-3-yl)pyridin-2-yl)piperidin-4-yl)methyl)piperidin-4-yl)-N-methyl-1H-indole-6-carboxamide (18)

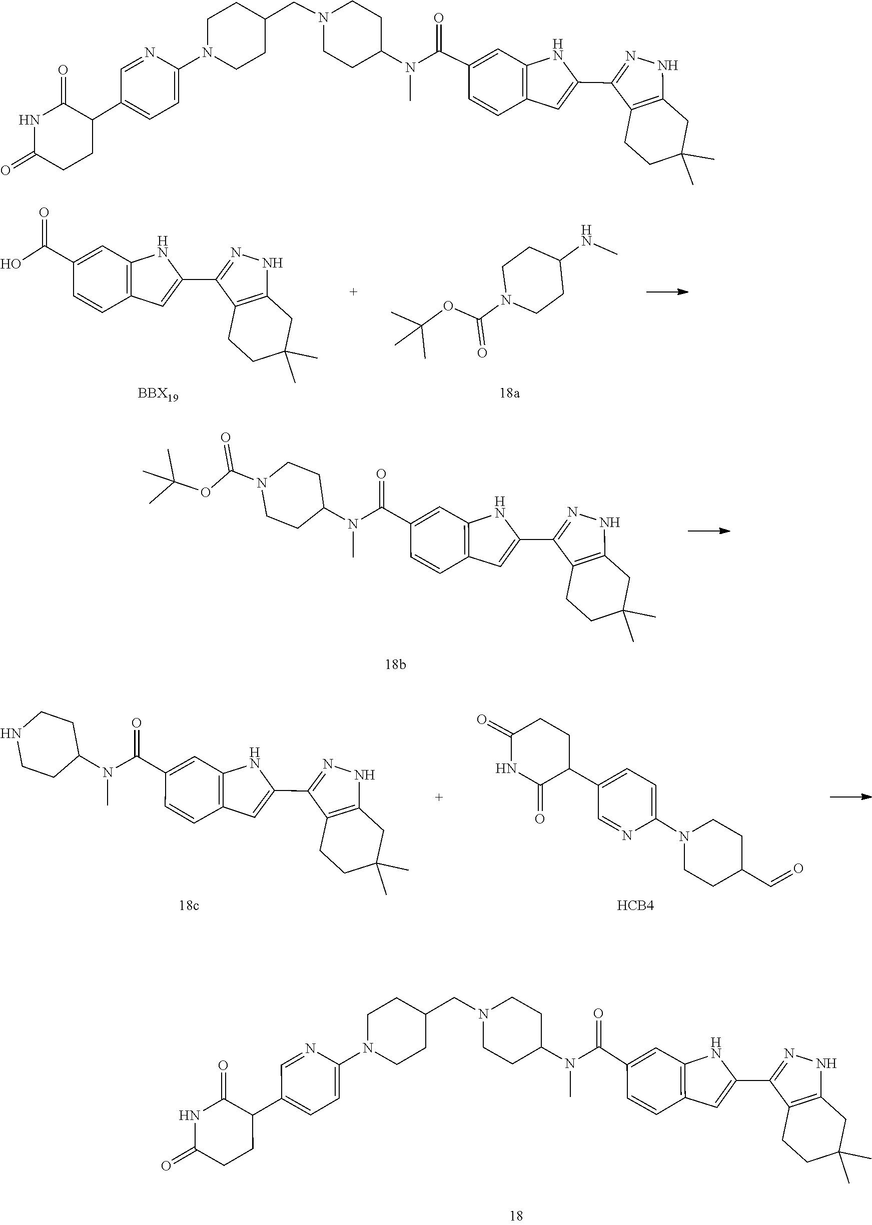

Step 1: To a solution of 2-(6,6-dimethyl-1,4,5,7-tetrahydroindazol-3-yl)-1H-indole-6-carboxylic acid (BBX₁₉) (15.00 mg, 0.05 mmol) and [(dimethylamino)({[1,2,3]triazolo[4,5-b]pyridin-3-yloxy})methylidene]dimethylazanium; hexafluoro-lambda5-phosphanuide (HATU) (18.44 mg, 0.05 mmol) in 0.3 DMF was added N,N-diisopropylethylamine (33.87 μL, 25.07 mg, 0.19 mmol), the solution was stirred for 5 minutes, then tert-butyl 4-(methylamino)piperidine-1-carboxylate (18a) (10.39 mg, 0.05 mmol) in 0.3 ml DMF was added, the reaction mixture was stirred for 10 minutes. LCMS indicated the completion of the reaction. Diluted the reaction mixture with 25 ml EtOAc, the solution was washed by water twice, dried over Na₂SO₄, concentrated. The crude product was purified on ISCO silica gel column, eluting with 0-10% MeOH in DCM to afford tert-butyl 4-[N-methyl2-(6,6-dimethyl-1,4,5,7-tetrahydroindazol-3-yl)-1H-indole-6-amido]piperidine-1-carboxylate (18b) (0.016 g, 65%). LCMS: C₂₉H₃₉N₅O₃ requires: 505.7, found: m/z=506.7 [M+H]⁺

Step 2: tert-butyl 4-[N-methyl2-(6,6-dimethyl-1,4,5,7-tetrahydroindazol-3-yl)-1H-indole-6-amido]piperidine-1-carboxylate (18b) (0.016 g, 0.03 mmol) was treated with 10 ml 5% TFA in HFIP for 30 minutes. The solvent was evaporated under reduced pressure; the product was used to the next step without purification. LCMS: C₂₄H₃₁N₅O requires: 405.6, found: m/z=406.6 [M+H]⁺.

Step 3: To a solution of 1-(5-(2,6-dioxopiperidin-3-yl)pyridin-2-yl)piperidine-4-carbaldehyde (18c) (10.00 mg, 0.03 mmol) and 2-(6,6-dimethyl-1,4,5,7-tetrahydroindazol-3-yl)-N-methyl-N-(piperidin-4-yl)-1H-indole-6-carboxamide (HCB4) (13.4 mg, 0.03 mmol) in 0.5 ml DMSO, N,N-diisopropylethylamine (23 μL, 0.13 mmol) in 1 ml DCM was added, then sodium triacetoxyborohydride (21.1 mg, 0.1 mmol) was added, the solution was stirred for 1 hr. then quenched with 1 ml of 10% NaHCO₃ solution, the solution was extracted with 30 ml of 5% MeOH in DCM. The organic layer was dried over Na₂SO₄, concentrated. The crude product was purified on ISCO silica gel column eluting with 2-20% MeOH in DCM to afford 2-(6,6-dimethyl-4,5,6,7-tetrahydro-1H-indazol-3-yl)-N-(1-((1-(5-(2,6-dioxopiperidin-3-yl)pyridin-2-yl)piperidin-4-yl)methyl)piperidin-4-yl)-N-methyl-1H-indole-6-carboxamide (18) (16 mg, 0.02 mmol, 66%). ¹H NMR (500 MHz, DMSO-d₆) δ 12.58 (s, 1H), 11.42 (s, 1H), 10.81 (s, 1H), 7.94 (d, J=2.5 Hz, 1H), 7.54 (d, J=8.1 Hz, 1H), 7.43 (s, 1H), 7.36 (dd, J=8.8, 2.5 Hz, 1H), 6.99 (d, J=8.1 Hz, 1H), 6.79 (d, J=8.9 Hz, 1H), 6.63 (s, 1H), 4.25 (d, J=12.7 Hz, 2H), 3.72 (dd, J=12.1, 4.9 Hz, 1H), 3.01 (s, 1H), 2.86 (s, 3H), 2.71 (ddt, J=23.3, 17.2, 8.8 Hz, 5H), 2.55 (d, J=2.5 Hz, 1H), 2.43 (s, 2H), 2.21 (dtt, J=29.3, 12.5, 5.5 Hz, 3H), 2.08-1.83 (m, 4H), 1.75 (d, J=12.5 Hz, 3H), 1.71-1.54 (m, 4H), 1.41 (s, 2H), 1.26 (d, J=9.6 Hz, 6H), 1.02 (s, 6H). LCMS: C₄₀H₅₀N₈O₃ requires: 690.4, found: m/z=691.8 [M+H]⁺.

### Example 54. 3-(6-(4-((1-(2-(6,6-dimethyl-4,5,6,7-tetrahydro-1H-indazol-3-yl)-1H-indole-6-carbonyl)piperidin-4-yl)methyl)piperidin-1-yl)pyridin-3-yl)piperidine-2,6-dione (19)

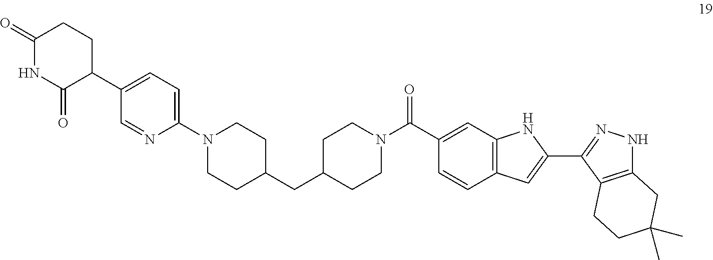 [SMILES: CC1(C)CCc2c(-c3cc4ccc(C(=O)N5CCC(CC6CCN(c7ccc(C8CCC(=O)NC8=O)cn7)CC6)CC5)cc4[nH]3)n[nH]c2C1]

Using general procedure 1, 2-(6,6-dimethyl-4,5,6,7-tetrahydro-1H-indazol-3-yl)-1H-indole-6-carboxylic acid (17 mg, 0.05 mmol) was treated with 3-(6-(4-(piperidin-4-ylmethyl)piperidin-1-yl)pyridin-3-yl)piperidine-2,6-dione (16 mg, 0.04 mmol) to afford the desired product (9.3 mg, 0.01 mmol, 30%). ¹H NMR (500 MHz, (CD₃)₂SO) δ 11.40 (s, 1H), 10.91 (s, 1H), 7.86 (s, 1H), 7.77 (s, 1H), 7.52 (d, J=8.2 Hz, 1H), 7.43 (s, 1H), 7.26 (s, 1H), 6.98 (d, J=8.2 Hz, 1H), 6.61 (s, 1H), 4.18 (d, J=13.0 Hz, 2H), 3.87 (s, 1H), 3.04 (t, J=64.3 Hz, 4H), 2.68 (d, J=5.4 Hz, 3H), 2.56 (d, J=17.7 Hz, 1H), 2.42 (s, 2H), 2.38-2.19 (m, 1H), 1.97 (s, 1H), 1.79 (d, J=12.9 Hz, 2H), 1.69 (s, 6H), 1.57 (d, J=7.0 Hz, 3H), 1.31 (q, J=7.4 Hz, 1H), 1.20 (s, 3H), 1.11 (dd, J=25.4, 12.0 Hz, 2H), 1.01 (s, 6H), 0.94 (t, J=7.3 Hz, 1H). LCMS: C₃₉H₄₇N₇O₃ requires: 662, found: m/z=663 [M+H]⁺.

### Example 55. 3-(6-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)pyridin-3-yl)piperidine-2,6-dione (20)

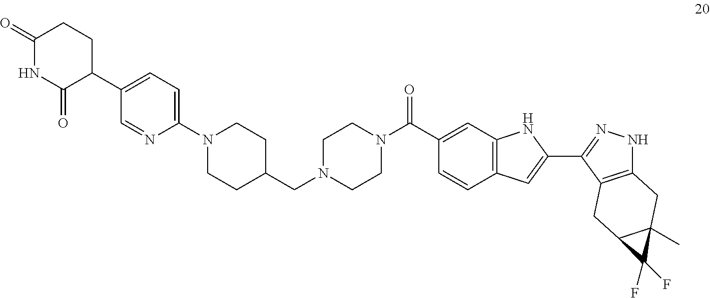 [SMILES: C[C@@]12Cc3[nH]nc(-c4cc5ccc(C(=O)N6CCN(CC7CCN(c8ccc(C9CCC(=O)NC9=O)cn8)CC7)CC6)cc5[nH]4)c3C[C@@H]1C2(F)F]

Using general procedure 2, (2-((4aS,5aR)-5,5-difluoro-5a-methyl-1,4,4a,5,5a,6-hexahydrocyclopropa[f]indazol-3-yl)-1H-indol-6-yl)(piperazin-1-yl)methanone (20 mg, 0.05 mmol) was treated with 1-(5-(2,6-dioxopiperidin-3-yl)pyridin-2-yl)piperidine-4-carbaldehyde (15 mg, 0.05 mmol) to afford the desired product (16 mg, 0.02 mmol, 48%). ¹H NMR (500 MHz, CD₃CN) δ 10.01 (d, J=30.6 Hz, 1H), 8.87 (s, 1H), 7.84-7.77 (m, 2H), 7.64 (d, J=8.1 Hz, 1H), 7.55 (s, 1H), 7.18 (d, J=9.4 Hz, 1H), 7.14 (dd, J=8.2, 1.4 Hz, 1H), 6.72 (d, J=3.5 Hz, 1H), 4.10 (d, J=13.5 Hz, 2H), 3.80 (dd, J=12.8, 5.1 Hz, 1H), 3.53 (s, 3H), 3.25-3.06 (m, 5H), 3.00 (d, J=6.8 Hz, 2H), 2.82 (dd, J=16.8, 3.3 Hz, 1H), 2.70-2.64 (m, 2H), 2.30-2.05 (m, 2H), 2.03-1.96 (m, 2H), 1.81-1.74 (m, 1H), 1.41 (s, 5H). LCMS: C₃₈H₄₂F₂N₈O₃ requires: 696, found: m/z=697 [M+H]⁺.

### Example 56. (4aS,5aR)—N-(1-((1S)-(1-(1-(5-(2,6-dioxopiperidin-3-yl)pyridin-2-yl)azetidine-3-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (21)

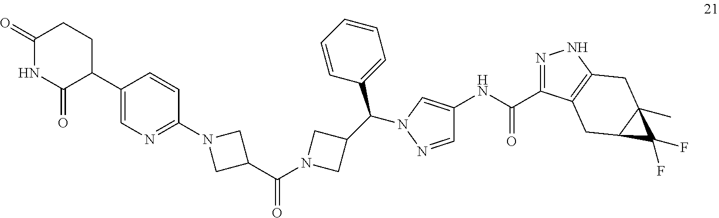 [SMILES: C[C@@]12Cc3[nH]nc(C(=O)Nc4cnn([C@H](c5ccccc5)C5CN(C(=O)C6CN(c7ccc(C8CCC(=O)NC8=O)cn7)C6)C5)c4)c3C[C@@H]1C2(F)F]

Using general procedure 4, starting from (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (23 mg, 0.05 mmol) was treated with 1-(5-(2,6-dioxopiperidin-3-yl)pyridin-2-yl)azetidine-3-carboxylic acid (15 mg, 0.05 mmol) to afford the desired product (20 mg, 0.03 mmol, 54%). ¹H NMR (500 MHz, CD₃CN) δ 8.90 (s, 1H), 8.82 (s, 1H), 8.02 (d, J=4.9 Hz, 1H), 7.78-7.70 (m, 2H), 7.63 (s, 1H), 7.41-7.30 (m, 5H), 6.71 (dd, J=9.2, 5.4 Hz, 1H), 5.57-5.51 (m, 1H), 5.45 (s, 1H), 4.42-4.18 (m, 5H), 4.14-4.01 (m, 1H), 3.99-3.89 (m, 1H), 3.77 (dp, J=11.8, 6.7, 5.9 Hz, 3H), 3.69-3.51 (m, 2H), 3.16 (d, J=17.6 Hz, 1H), 3.12-3.02 (m, 2H), 2.81-2.74 (m, 1H), 2.70-2.62 (m, 2H), 2.17 (dtd, J=13.5, 11.7, 10.1, 5.6 Hz, 1H), 2.14-2.05 (m, 1H), 1.66 (dd, J=15.2, 6.9 Hz, 1H), 1.37 (s, 3H). LCMS: C₃₇H₃₇F₂N₉O₄ requires: 709, found: m/z=710 [M+H]⁺.

### Example 57. (4aS,5aR)—N-(1-((1-(1-(5-(2,6-dioxopiperidin-3-yl)pyridin-2-yl)piperidine-4-carbonyl)azetidin-3-yl)methyl)-1H-pyrazol-4-yl)-5,5-difluoro-5a-methyl-1,4,4a,5,5a,6-hexahydrocyclopropa[f]indazole-3-carboxamide (22)

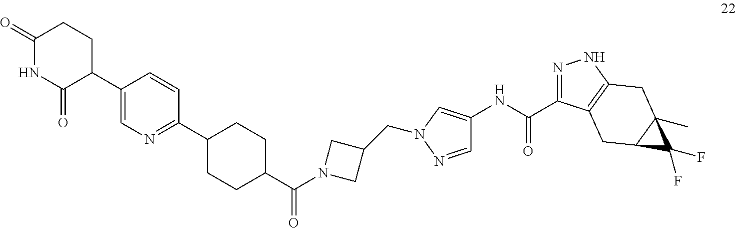 [SMILES: C[C@@]12Cc3[nH]nc(C(=O)Nc4cnn(CC5CN(C(=O)C6CCC(c7ccc(C8CCC(=O)NC8=O)cn7)CC6)C5)c4)c3C[C@@H]1C2(F)F]

Using general procedure 4, (4aS,5aR)—N-(1-(azetidin-3-ylmethyl)-1H-pyrazol-4-yl)-5,5-difluoro-5a-methyl-1,4,4a,5,5a,6-hexahydrocyclopropa[f]indazole-3-carboxamide (27 mg, 0.07 mmol) was treated with 1-(5-(2,6-dioxopiperidin-3-yl)pyridin-2-yl)piperidine-4-carboxylic acid (24 mg, 0.07 mmol) to afford the desired product (19 mg, 0.03 mmol, 38%). ¹H NMR (500 MHz, CD₃CN) δ 8.89 (d, J=4.9 Hz, 1H), 8.83 (d, J=11.0 Hz, 1H), 8.02 (s, 1H), 7.82-7.75 (m, 2H), 7.59 (s, 1H), 7.17 (dd, J=9.6, 2.7 Hz, 1H), 4.31 (d, J=7.0 Hz, 2H), 4.25 (t, J=8.5 Hz, 1H), 4.07-4.00 (m, 3H), 3.95 (t, J=9.2 Hz, 1H), 3.79 (dd, J=12.8, 5.1 Hz, 1H), 3.70 (dd, J=10.0, 5.4 Hz, 1H), 3.34-3.24 (m, 2H), 3.18 (d, J=17.6 Hz, 1H), 3.14-3.04 (m, 3H), 2.79 (dd, J=17.3, 3.4 Hz, 1H), 2.68 (d, J=3.0 Hz, 1H), 2.62 (tt, J=10.1, 4.1 Hz, 1H), 2.27-2.07 (m, 2H), 1.83 (d, J=14.1 Hz, 2H), 1.76-1.63 (m, 3H), 1.37 (d, J=2.5 Hz, 3H). LCMS: C₃₃H₃₇F₂N₉O₄ requires: 662, found: m/z=663 [M+H]⁺.

### Example 58. (4aS,5aR)—N-(1-((1-(1-(5-(2,6-dioxopiperidin-3-yl)pyridin-2-yl)azetidine-3-carbonyl)azetidin-3-yl)methyl)-1H-pyrazol-4-yl)-5,5-difluoro-5a-methyl-1,4,4a,5,5a,6-hexahydrocyclopropa[f]indazole-3-carboxamide (23)

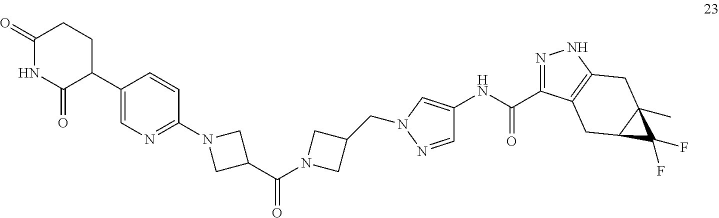 [SMILES: C[C@@]12Cc3[nH]nc(C(=O)Nc4cnn(CC5CN(C(=O)C6CN(c7ccc(C8CCC(=O)NC8=O)cn7)C6)C5)c4)c3C[C@@H]1C2(F)F]

Using general procedure 4, (4aS,5aR)—N-(1-(azetidin-3-ylmethyl)-1H-pyrazol-4-yl)-5,5-difluoro-5a-methyl-1,4,4a,5,5a,6-hexahydrocyclopropa[f]indazole-3-carboxamide (20 mg, 0.06 mmol) was treated with 1-(5-(2,6-dioxopiperidin-3-yl)pyridin-2-yl)azetidine-3-carboxylic acid (16 mg, 0.06 mmol) to afford the desired product (19 mg, 0.03 mmol, 55%). ¹H NMR (500 MHz, CD₃CN) δ 8.91 (s, 1H), 8.83 (s, 1H), 8.01 (d, J=2.1 Hz, 1H), 7.74 (ddd, J=8.9, 5.6, 2.5 Hz, 1H), 7.67 (d, J=2.3 Hz, 1H), 7.61 (s, 1H), 6.72 (dt, J=9.5, 2.4 Hz, 1H), 4.39-4.29 (m, 5H), 4.26 (dd, J=9.1, 5.6 Hz, 1H), 4.16 (t, J=8.5 Hz, 1H), 4.01 (t, J=9.2 Hz, 1H), 3.93 (dd, J=8.7, 5.5 Hz, 1H), 3.82-3.73 (m, 2H), 3.58 (ddd, J=14.6, 8.6, 6.1 Hz, 1H), 3.21-3.05 (m, 4H), 2.79 (dd, J=17.3, 3.5 Hz, 1H), 2.70-2.65 (m, 1H), 2.15 (dddd, J=31.0, 13.0, 9.5, 3.9 Hz, 2H), 1.72-1.63 (m, 1H), 1.38 (d, J=2.6 Hz, 3H). LCMS: C₃₁H₃₃F₂N₉O₄ requires: 634, found: m/z=635 [M+H]⁺.

### Example 59. 3-(6-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)pyridin-3-yl)piperidine-2,6-dione (24)

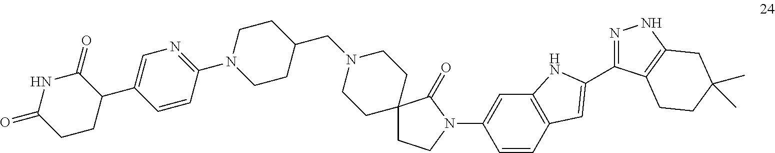 [SMILES: CC1(C)CCc2c(-c3cc4ccc(N5CCC6(CCN(CC7CCN(c8ccc(C9CCC(=O)NC9=O)cn8)CC7)CC6)C5=O)cc4[nH]3)n[nH]c2C1]

3-(6-(4-(2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)pyridin-3-yl)piperidine-2,6-dione (0.0221 g, 44.0%) was prepared by procedures analogous to Example 67 starting from 2-[2-(6,6-dimethyl-1,4,5,7-tetrahydroindazol-3-yl)-1H-indol-6-yl]-2,8-diazaspiro[4.5]decan-1-one (20.60 mg, 0.05 mmol) 1-(5-(2,6-dioxopiperidin-3-yl)pyridin-2-yl)piperidine-4-carbaldehyde (21.1 mg, 0.07 mmol). ¹H NMR (500 MHz, DMSO-d₆) δ 12.51 (s, 1H), 11.28 (s, 1H), 10.83 (s, 1H), 8.94 (s, 1H), 7.97 (d, J=2.4 Hz, 1H), 7.74 (d, J=9.3 Hz, 1H), 7.50 (d, J=8.6 Hz, 1H), 7.42 (d, J=8.7 Hz, 1H), 7.25 (d, J=8.5 Hz, 1H), 6.87 (d, J=9.0 Hz, 1H), 6.56 (s, 1H), 4.31 (d, J=13.0 Hz, 2H), 3.93-3.85 (m, 2H), 3.75 (dd, J=12.1, 4.9 Hz, 1H), 3.60 (d, J=11.7 Hz, 2H), 3.48 (s, 1H), 3.13-3.04 (m, 4H), 2.86 (t, J=12.3 Hz, 2H), 2.75-2.64 (m, 3H), 2.42 (s, 2H), 2.25-2.08 (m, 5H), 2.03-1.95 (m, 1H), 1.87-1.80 (m, 4H), 1.59 (t, J=6.3 Hz, 2H), 1.35-1.16 (m, 3H), 1.02 (s, 6H). LCMS: C₄₁H₅₀N₈O₃ requires: 702, found: m/z=703 [M+H]⁺.

### Example 60. N-(2-((4aS,5aR)-5,5-difluoro-5a-methyl-1,4,4a,5,5a,6-hexahydrocyclopropa[f]indazol-3-yl)-1H-indol-6-yl)-3-(1-(5-(2,6-dioxopiperidin-3-yl)pyridin-2-yl)pyrrolidin-3-yl)-N-methylpropanamide (25)

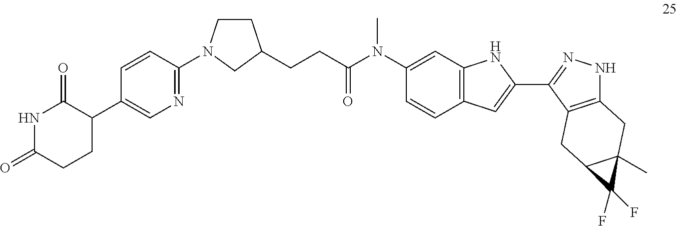 [SMILES: CN(C(=O)CCC1CCN(c2ccc(C3CCC(=O)NC3=O)cn2)C1)c1ccc2cc(-c3n[nH]c4c3C[C@@H]3C(F)(F)[C@]3(C)C4)[nH]c2c1]

A mixture of N-[2-[(4aS,5aR)-5,5-difluoro-5a-methyl-1-(oxan-2-yl)-4H,4aH,6H-cyclopropa[f]indazol-3-yl]-1H-indol-6-yl]-3-[1-[5-(2,6-dioxopiperidin-3-yl)pyridin-2-yl]pyrrolidin-3-yl]-N-methylpropanamide (25 mg, 0.034 mmol, 1 equiv) and HCl (4 M in dioxane, 3 mL) was stirred for one hour at room temperature. The resulting mixture was concentrated under vacuum to afford the desired product (2.3 mg, 4.52%) as a white solid. MS (ESI) calc'd for C₃₅H₃₇F₂N₇O₃ [M+1]⁺: 642.2, found, 642.2. ¹H NMR (300 MHz, (CD₃)₂CO) δ 10.83 (s, 1H), 9.58 (s, 1H), 7.97-7.91 (m, 1H), 7.68-7.59 (m, 1H), 7.48-7.35 (m, 2H), 7.01-6.91 (m, 1H), 6.76-6.69 (m, 1H), 6.42-6.33 (m, 1H), 3.82-3.71 (m, 1H), 3.53 (s, 2H), 3.40-3.29 (m, 1H), 3.26 (s, 3H), 3.24-3.10 (m, 4H), 2.77-2.56 (m, 3H), 2.26-2.14 (m, 6H), 1.84-1.68 (m, 2H), 1.60-1.44 (m, 1H), 1.48-1.40 (m, 3H), 1.30 (s, 1H). LCMS: C₃₅H₃₇F₂N₇O₃ requires: 641, found: m/z=642 [M+H]⁺.

### Example 61. 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-3-yl)piperidine-2,6-dione (26)

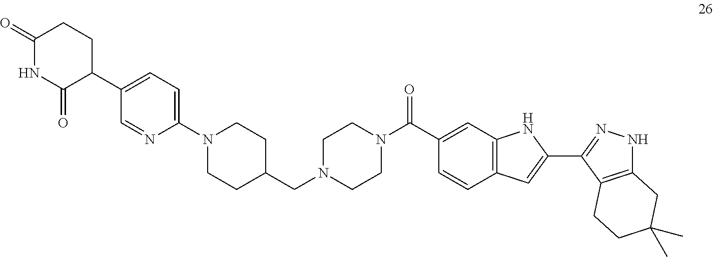 [SMILES: CC1(C)CCc2c(-c3cc4ccc(C(=O)N5CCN(CC6CCN(c7ccc(C8CCC(=O)NC8=O)cn7)CC6)CC5)cc4[nH]3)n[nH]c2C1]

Using general procedure 2, (2-(6,6-dimethyl-4,5,6,7-tetrahydro-1H-indazol-3-yl)-1H-indol-6-yl)(piperazin-1-yl)methanone (103 mg, 0.27 mmol) was treated with 1-(5-(2,6-dioxopiperidin-3-yl)pyridin-2-yl)piperidine-4-carbaldehyde (99 mg, 0.33 mmol) to afford the desired product (141 mg, 0.21 mmol, 76%). ¹H NMR (500 MHz, CDCl₃) δ 10.23 (s, 1H), 10.07 (s, 1H), 8.06 (d, J=2.5 Hz, 1H), 7.59 (d, J=8.1 Hz, 1H), 7.51 (s, 1H), 7.31 (dd, J=8.8, 2.5 Hz, 1H), 7.13 (dd, J=8.1, 1.4 Hz, 1H), 6.73-6.70 (m, 1H), 6.67 (d, J=8.9 Hz, 1H), 4.29 (d, J=12.7 Hz, 2H), 3.68 (dd, J=10.4, 5.2 Hz, 1H), 2.89-2.79 (m, 2H), 2.79-2.74 (m, 2H), 2.73-2.65 (m, 1H), 2.44 (s, 2H), 2.25 (qt, J=14.7, 7.7 Hz, 3H), 1.85 (d, J=13.0 Hz, 2H), 1.77 (s, 1H), 1.65 (t, J=6.3 Hz, 2H), 1.24 (d, J=11.9 Hz, 4H), 1.05 (s, 6H). LCMS: C₃₈H₄₆N₈O₃ requires: 663, found: m/z=664 [M+H]⁺.

### Example 62. N-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-2-(1-(5-(2,6-dioxopiperidin-3-yl)pyridin-2-yl)piperidin-4-yl)-N-methylacetamide (27)

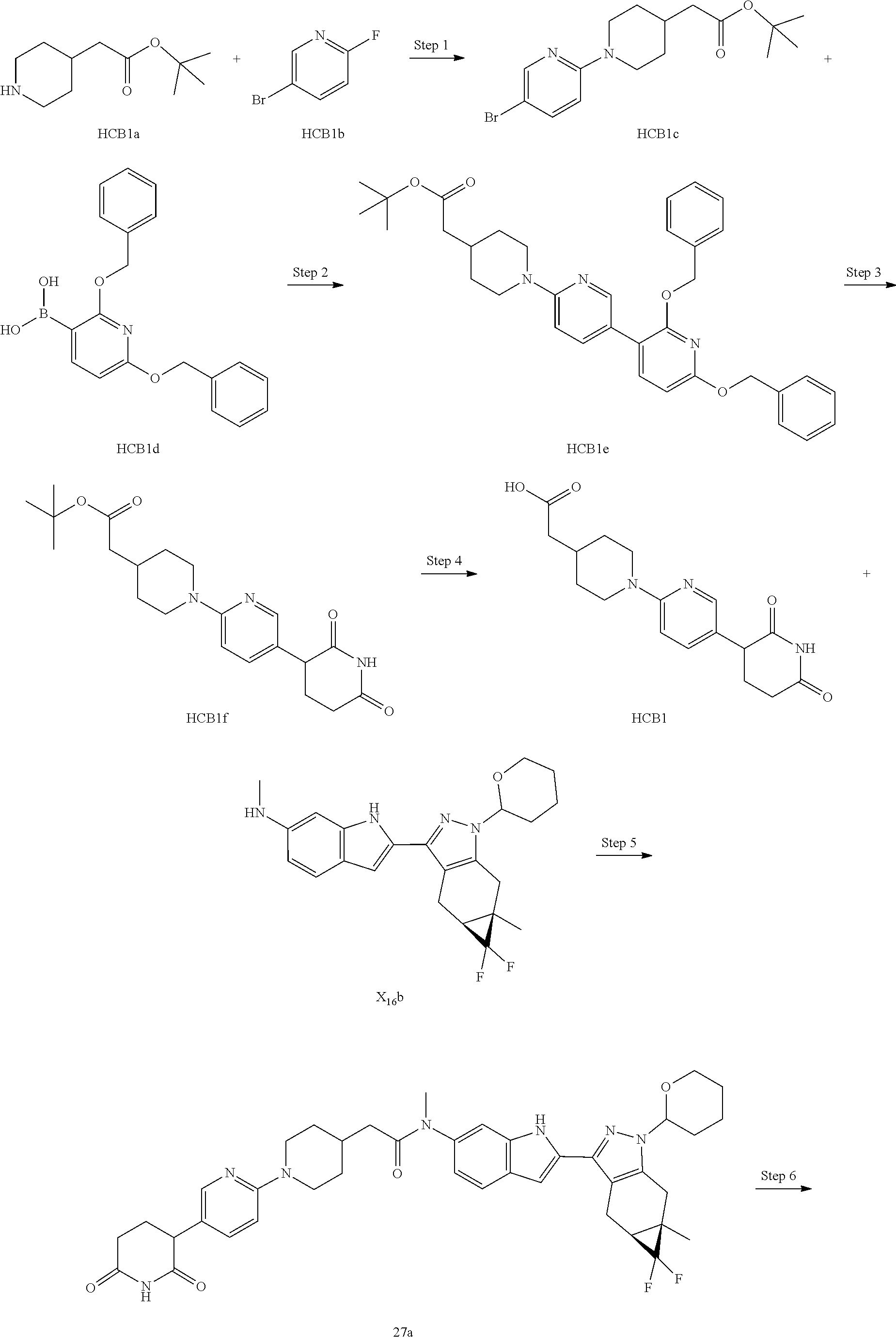

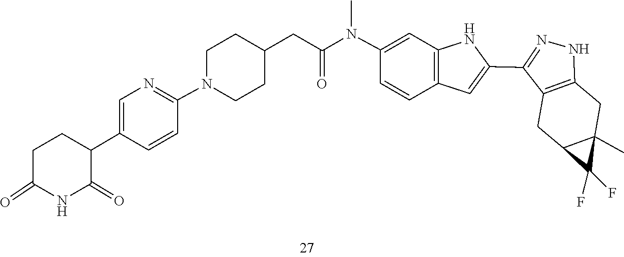 [SMILES: CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1]

### Step 1: tert-butyl 2-[1-(5-bromopyridin-2-yl)piperidin-4-yl]acetate (HCB1c)

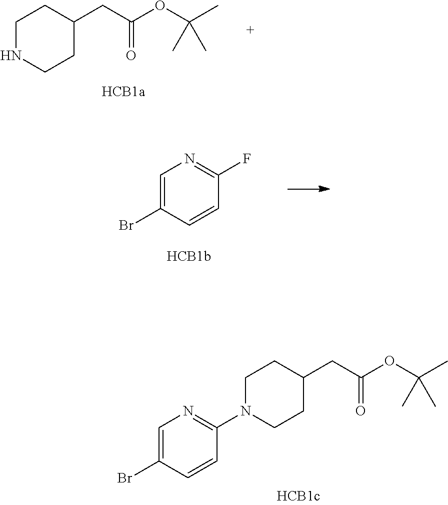 [SMILES: CC(C)(C)OC(=O)CC1CCNCC1 ; Fc1ccc(Br)cn1 ; CC(C)(C)OC(=O)CC1CCN(c2ccc(Br)cn2)CC1]

tert-butyl 2-(piperidin-4-yl)acetate (765 mg, 3.84 mmol), 5-bromo-2-fluoropyridine (0.40 mL, 676 mg, 3.84 mmol), and potassium carbonate (1.06 g, 7.68 mmol) were stirred in DMF (10.00 mL) overnight. The mixture was heated at 50° C. for six hours. The mixture was stirred at room temperature for five days, transferred to a separatory funnel with ethyl acetate, and then washed with two portions of water. The organic layer was dried over Na₂SO₄, filtered, and concentrated. The crude residue was purified by flash chromatography on a 40 g column e (gradient elution with zero to 20% ethyl acetate:hexanes) to provide tert-butyl 2-[1-(5-bromopyridin-2-yl)piperidin-4-yl]acetate (0.655 g, 48.0%). LCMS: C₁₆H₂₃BrN₂O₂ requires: 355, found: m/z=356 [M+H]⁺.

### Step 2: tert-butyl 2-{1-[2′,6′-bis(benzyloxy)-[3,3′-bipyridin]-6-yl]piperidin-4-yl}acetate (HCB1e)

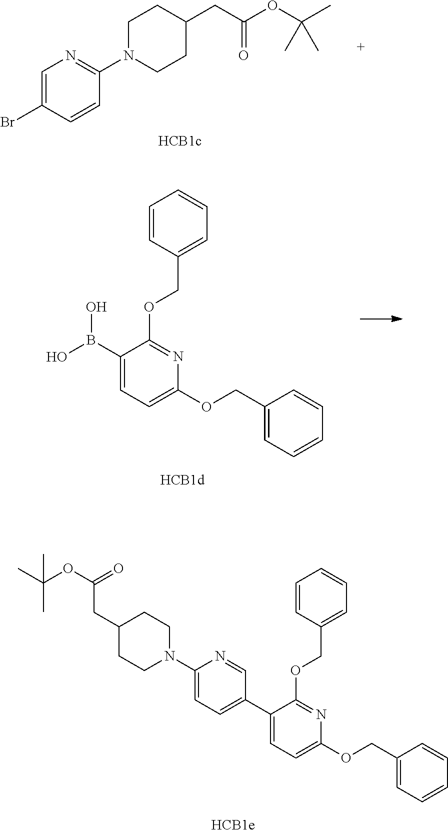

A mixture of 2,6-bis(benzyloxy)pyridin-3-ylboronic acid (495 mg, 1.48 mmol), tert-butyl 2-[1-(5-bromopyridin-2-yl)piperidin-4-yl]acetate (525 mg, 1.48 mmol), tetrakis(triphenylphosphine)palladium(0) (171 mg, 0.15 mmol), and potassium carbonate (408 mg, 2.96 mmol) in water (1.00 mL) and THF (3.00 mL) was microwaved at 120° C. for forty minutes. The water layer was removed by pipette. The organic layer was concentrated and then purified by flash chromatography on a 40 g column (gradient elution with zero to 35% ethyl acetate:hexanes) to provide tert-butyl 2-{1-[2′,6′-bis(benzyloxy)-[3,3′-bipyridin]-6-yl]piperidin-4-yl}acetate (0.303 g, 36.2%). LCMS: C₃₅H₃₉N₃O₄ requires: 565, found: m/z=566 [M+H]⁺.

### Step 3: tert-butyl 2-{1-[5-(2,6-dioxopiperidin-3-yl)pyridin-2-yl]piperidin-4-yl}acetate (HCB10

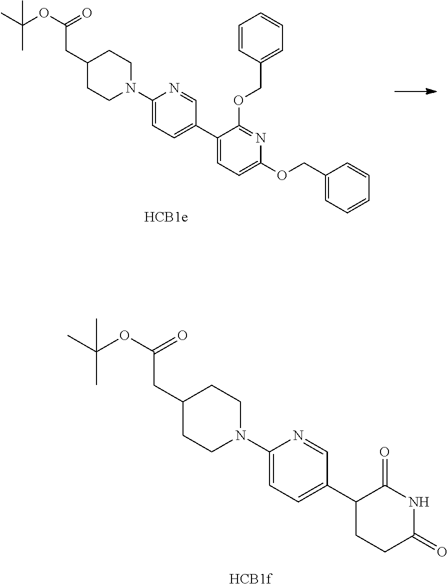 [SMILES: CC(C)(C)OC(=O)CC1CCN(c2ccc(-c3ccc(OCc4ccccc4)nc3OCc3ccccc3)cn2)CC1 ; CC(C)(C)OC(=O)CC1CCN(c2ccc(C3CCC(=O)NC3=O)cn2)CC1]

tert-butyl 2-{1-[2′,6′-bis(benzyloxy)-[3,3′-bipyridin]-6-yl]piperidin-4-yl}acetate (303 mg, 0.54 mmol) was suspended in EtOH (9.00 mL). THF (4 mL) was added along with 10% Pd/C (303 mg). The mixture was stirred under a balloon of H₂ for two hours. The mixture was diluted with THF (100 mL) and filtered through a pad of celite. The filtrate was concentrated and the crude residue was purified by flash chromatography on a 24 g column (gradient elution with zero to 10% MeOH:DCM) to provide tert-butyl 2-{1-[5-(2,6-dioxopiperidin-3-yl)pyridin-2-yl]piperidin-4-yl}acetate (0.152 g, 73.2%). LCMS: C₂₁H₂₉N₃O₄ requires: 387, found: m/z=388 [M+H]⁺.

### Step 4: {1-[5-(2,6-dioxopiperidin-3-yl)pyridin-2-yl]piperidin-4-yl}acetic acid (HCB1)

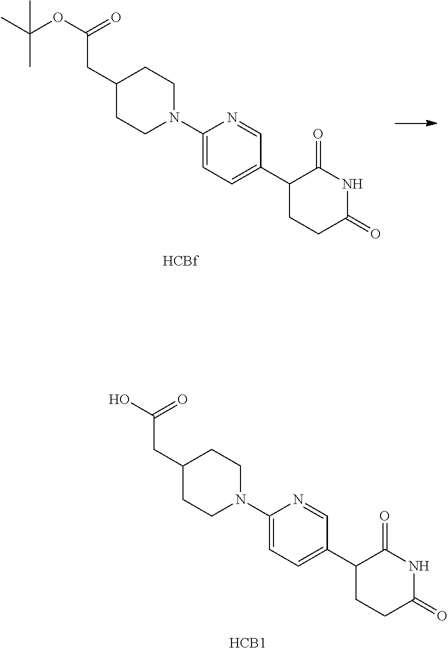 [SMILES: CC(C)(C)OC(=O)CC1CCN(c2ccc(C3CCC(=O)NC3=O)cn2)CC1 ; O=C(O)CC1CCN(c2ccc(C3CCC(=O)NC3=O)cn2)CC1 ; "HCB1"]

tert-butyl 2-{1-[5-(2,6-dioxopiperidin-3-yl)pyridin-2-yl]piperidin-4-yl}acetate (29.00 mg, 0.07 mmol) was stirred in DCM (0.50 mL) and 4 M HCl solution in dioxane (0.50 mL, 0.07 g, 2.00 mmol). After stirring overnight, the mixture was concentrated in vacuo to provide {1-[5-(2,6-dioxopiperidin-3-yl)pyridin-2-yl]piperidin-4-yl}acetic acid (0.0248 g, 100%). LCMS: C₁₇H₂₁N₃O₄ requires: 331, found: m/z=332 [M+H]⁺.

### Step 5: N-{2-[(4aS,5aR)-5,5-difluoro-5a-methyl-1-(oxan-2-yl)-4H,4aH,6H-cyclopropa[f]indazol-3-yl]-1H-indol-6-yl}-2-(1-{5-[(3RS)-2,6-dioxopiperidin-3-yl]pyridin-2-yl}piperidin-4-yl)-N-methylacetamide (27a)

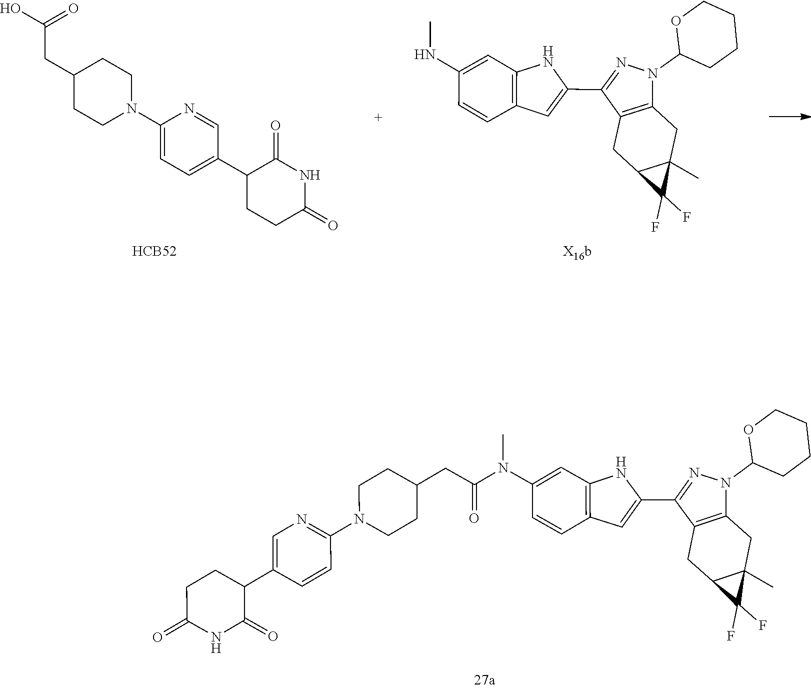 [SMILES: "HCB52" ; O=C(O)CC1CCN(c2ccc(C3CCC(=O)NC3=O)cn2)CC1 ; CNc1ccc2cc(-c3nn(C4CCCCO4)c4c3C[C@@H]3C(F)(F)[C@]3(C)C4)[nH]c2c1 ; "X16b" ; CN(C(=O)CC1CCN(c2ccc(C3CCC(=O)NC3=O)cn2)CC1)c1ccc2cc(-c3nn(C4CCCCO4)c4c3C[C@@H]3C(F)(F)[C@]3(C)C4)[nH]c2c1]

2-[(4aS,5aR)-5,5-difluoro-5a-methyl-1-(oxan-2-yl)-4H,4aH,6H-cyclopropa[f]indazol-3-yl]-N-methyl-1H-indol-6-amine (28.9 mg, 0.07 mmol) in DMF (2.00 mL) was added to a mixture of 2-(1-(5-(2,6-dioxopiperidin-3-yl)pyridin-2-yl)piperidin-4-yl)acetic acid (23.2 mg, 0.07 mmol), HATU (26.6 mg, 0.07 mmol), and N,N-diisopropylethylamine (0.05 mL, 0.04 g, 0.28 mmol) in DMF (2.00 mL). After stirring overnight, water was added and the mixture was extracted with ethyl acetate. The organic layer was concentrated and the crude residue was purified by preparative TLC eluted with 5% MeOH:DCM to provide N-{2-[(4aS,5aR)-5,5-difluoro-5a-methyl-1-(oxan-2-yl)-4H,4aH,6H-cyclopropa[f]indazol-3-yl]-1H-indol-6-yl}-2-(1-{5-[(3RS)-2,6-dioxopiperidin-3-yl]pyridin-2-yl}piperidin-4-yl)-N-methylacetamide (0.0428 g, 84.2%). LCMS: C₄₀H₄₅F₂N₇O₄ requires: 725, found: m/z=726 [M+H]⁺.

### Step 6: N-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-2-(1-(5-(2,6-dioxopiperidin-3-yl)pyridin-2-yl)piperidin-4-yl)-N-methylacetamide (27)

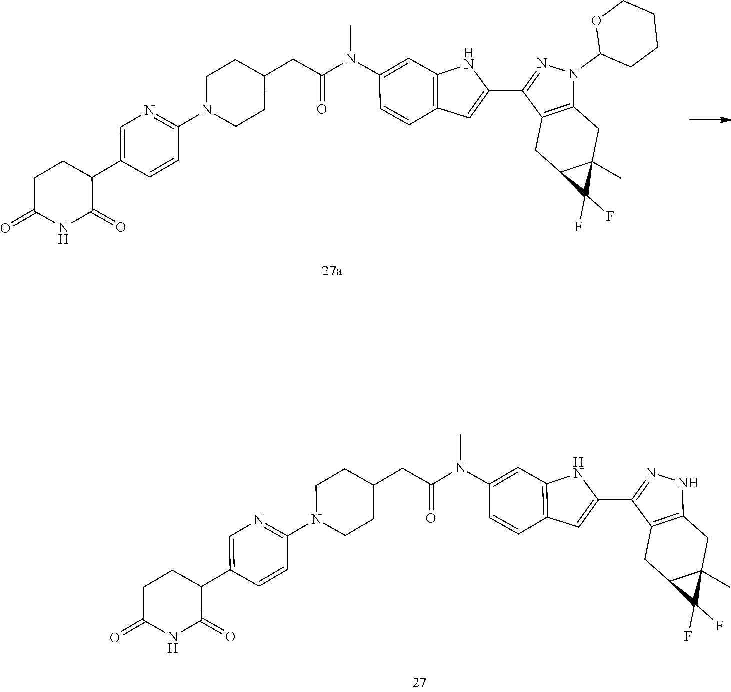 [SMILES: CN(C(=O)CC1CCN(c2ccc(C3CCC(=O)NC3=O)cn2)CC1)c1ccc2cc(-c3nn(C4CCCCO4)c4c3C[C@@H]3C(F)(F)[C@]3(C)C4)[nH]c2c1 ; CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1]

N-{2-[(4aS,5aR)-5,5-difluoro-5a-methyl-1-(oxan-2-yl)-4H,4aH,6H-cyclopropa[f]indazol-3-yl]-1H-indol-6-yl}-2-(1-{5-[(3RS)-2,6-dioxopiperidin-3-yl]pyridin-2-yl}piperidin-4-yl)-N-methylacetamide (42.8 mg, 0.06 mmol) and para-toluene sulfonate hydrate (0.02 mL, 22.4 mg, 0.12 mmol) were stirred in EtOH (2.00 mL) at 50° C. for one hour. The mixture was diluted with water then extracted twice with 10% MeOH:DCM. The combined organic layers were concentrated and the crude residue was purified by preparative TLC eluted with 10% MeOH:DCM to provide N-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-2-(1-(5-(2,6-dioxopiperidin-3-yl)pyridin-2-yl)piperidin-4-yl)-N-methylacetamide (0.0227 g, 59.4%). ¹H NMR (500 MHz, DMSO-d₆) δ 12.67 (s, 1H), 11.43 (s, 1H), 10.79 (s, 1H), 7.94-7.88 (m, 1H), 7.56 (d, J=8.2 Hz, 1H), 7.35-7.29 (m, 1H), 7.23 (s, 1H), 6.87 (d, J=8.3 Hz, 1H), 6.73 (d, J=8.7 Hz, 1H), 6.62 (s, 1H), 4.17 (d, J=12.8 Hz, 2H), 3.69 (dd, J=12.0, 4.9 Hz, 1H), 3.56 (d, J=3.5 Hz, 1H), 3.21 (s, 3H), 3.17-2.96 (m, 3H), 2.87-2.80 (m, 1H), 2.79-2.58 (m, 3H), 2.20-2.08 (m, 1H), 2.04-1.91 (m, 4H), 1.86 (dd, J=15.1, 6.9 Hz, 1H), 1.65 (d, J=12.7 Hz, 2H), 1.39 (s, 3H), 0.98-0.92 (m, 2H). LCMS: C₃₅H₃₇F₂N₇O₃ requires: 641, found: m/z=642 [M+H]⁺.

### Example 63. 3-(6-(4-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)piperidin-1-yl)pyridin-3-yl)piperidine-2,6-dione (28)

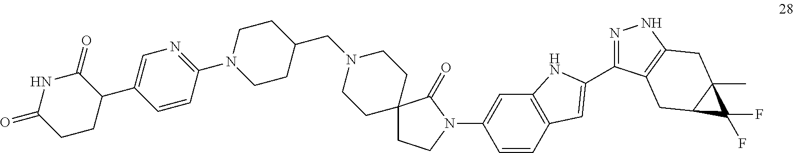 [SMILES: C[C@@]12Cc3[nH]nc(-c4cc5ccc(N6CCC7(CCN(CC8CCN(c9ccc(C%10CCC(=O)NC%10=O)cn9)CC8)CC7)C6=O)cc5[nH]4)c3C[C@@H]1C2(F)F]

3-(6-(4-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)piperidin-1-yl)pyridin-3-yl)piperidine-2,6-dione (0.0149 g, 33.4%) was prepared by procedures analogous to Example 67 starting from 3-(6-(4-(hydroxymethyl)piperidin-1-yl)pyridin-3-yl)piperidine-2,6-dione (18.7 mg, 0.06 mmol) and 2-{2-[(4aS,5aR)-5,5-difluoro-5a-methyl-1H,4H,4aH,6H-cyclopropa[f]indazol-3-yl]-1H-indol-6-yl}-2,8-diazaspiro[4.5]decan-1-one (22.0 mg, 0.05 mmol). ¹H NMR (500 MHz, DMSO-d₆) δ 12.65 (s, 1H), 11.30 (s, 1H), 10.85 (s, 1H), 9.16 (s, 1H), 7.97-7.93 (m, 1H), 7.78-7.72 (m, 1H), 7.54-7.48 (m, 1H), 7.39 (s, 1H), 7.26 (dd, J=8.4, 2.0 Hz, 1H), 6.90 (s, 1H), 6.59 (d, J=17.2 Hz, 1H), 4.31 (d, J=13.0 Hz, 2H), 3.93-3.85 (m, 2H), 3.79 (s, 1H), 3.62-3.56 (m, 2H), 3.48-3.44 (m, 1H), 3.17-2.97 (m, 7H), 2.97-2.80 (m, 1H), 2.75-2.65 (m, 1H), 2.57-2.53 (m, 2H), 2.22-2.12 (m, 5H), 2.02-1.95 (m, 1H), 1.89-1.80 (m, 5H), 1.40 (s, 3H), 1.27-1.23 (m, 3H). LCMS: C₄₁H₄₆F₂N₈O₃ requires: 736, found: m/z=737 [M+H]⁺.

### Example 64. (S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (29)

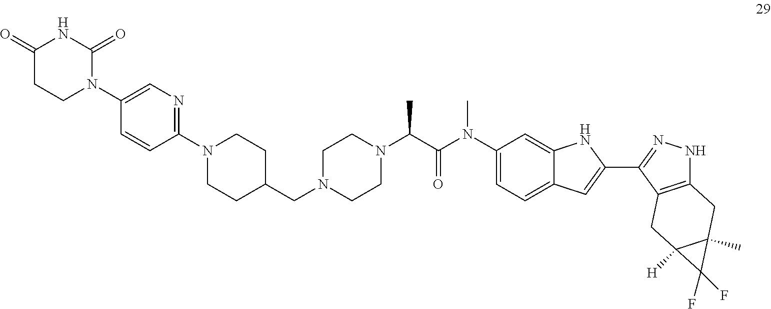 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(N4CCC(=O)NC4=O)cn3)CC2)CC1]

(S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (0.0250 g, 32.1%) was prepared by procedures analogous to Example 67 starting from 1-{6-[4-(hydroxymethyl)piperidin-1-yl]pyridin-3-yl}-1,3-diazinane-2,4-dione (30.0 mg, 0.10 mmol) and (2S)—N-{2-[(4aS,5aR)-5,5-difluoro-5a-methyl-1H,4H,4aH,6H-cyclopropa[f]indazol-3-yl]-1H-indol-6-yl}-N-methyl-2-(piperazin-1-yl)propanamide (46.9 mg, 0.10 mmol). ¹H NMR (500 MHz, DMSO-d₆) δ 12.73 (s, 1H), 11.47 (s, 1H), 10.35 (s, 1H), 9.22 (s, 1H), 8.08-8.02 (m, 1H), 7.60 (d, J=8.3 Hz, 1H), 7.53-7.47 (m, 1H), 7.32 (s, 1H), 6.96-6.86 (m, 2H), 6.65 (s, 1H), 4.29 (d, J=13.3 Hz, 2H), 3.70 (t, J=6.7 Hz, 2H), 3.53-3.25 (m, 3H), 3.22 (s, 3H), 3.19-2.74 (m, 12H), 2.74-2.59 (m, 3H), 2.07-2.04 (m, 1H), 1.92-1.84 (m, 1H), 1.83-1.67 (m, 3H), 1.45-1.32 (m, 4H), 1.22-1.18 (m, 2H), 1.09 (d, J=6.7 Hz, 3H). LCMS: C₄₀H₄₈F₂N₁₀O₃ requires: 754, found: m/z=755 [M+H]⁺.

### Example 65. (2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (30)

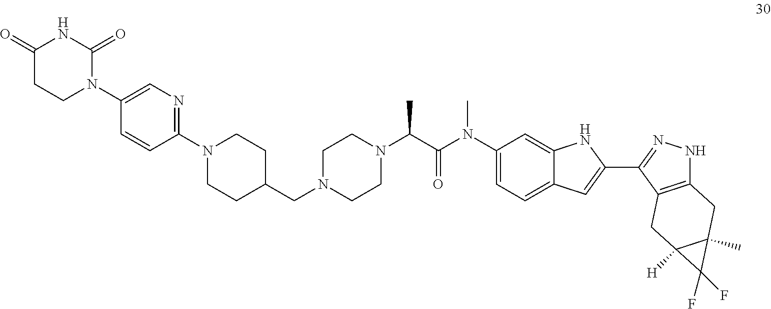 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(N4CCC(=O)NC4=O)cn3)CC2)CC1]

(2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (0.0266 g, 34.6%) was prepared by procedures analogous to Example 67 starting from 3-(6-(4-(hydroxymethyl)piperidin-1-yl)pyridin-3-yl)piperidine-2,6-dione (30.0 mg, 0.10 mmol) and (2S)—N-{2-[(4aS,5aR)-5,5-difluoro-5a-methyl-1H,4H,4aH,6H-cyclopropa[f]indazol-3-yl]-1H-indol-6-yl}-N-methyl-2-(piperazin-1-yl)propanamide (46.9 mg, 0.10 mmol). ¹H NMR (500 MHz, DMSO-d₆) δ 12.72 (s, 1H), 11.46 (s, 1H), 10.82 (s, 1H), 9.16 (s, 1H), 7.97-7.91 (m, 1H), 7.59 (d, J=8.3 Hz, 1H), 7.40-7.36 (m, 1H), 7.31 (s, 1H), 6.93 (d, J=8.3 Hz, 1H), 6.86-6.75 (m, 1H), 6.64 (s, 1H), 4.31-4.24 (m, 2H), 3.74 (dd, J=12.2, 5.0 Hz, 1H), 3.42 (t, J=11.7 Hz, 2H), 3.22 (s, 3H), 3.18-2.74 (m, 12H), 2.74-2.58 (m, 3H), 2.22-2.14 (m, 1H), 2.12-1.91 (m, 2H), 1.92-1.83 (m, 1H), 1.83-1.67 (m, 3H), 1.42-1.35 (m, 4H), 1.28-1.13 (m, 2H), 1.08 (d, J=6.8 Hz, 3H). LCMS: C₄₁H₄₉F₂N₉O₃ requires: 753, found: m/z=754 [M+H]⁺.

### Example 66. (S)—N-(2-(6,6-dimethyl-4,5,6,7-tetrahydro-1H-indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (31)

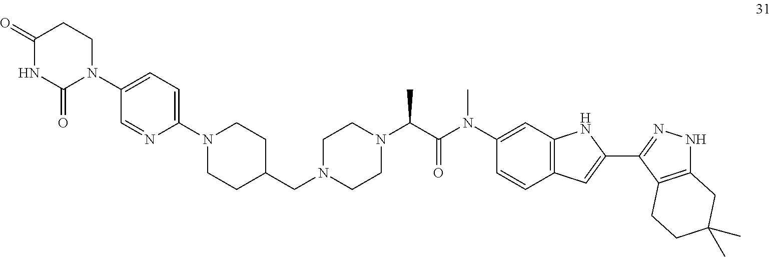 [SMILES: C[C@@H](C(=O)N(C)c1ccc2cc(-c3n[nH]c4c3CCC(C)(C)C4)[nH]c2c1)N1CCN(CC2CCN(c3ccc(N4CCC(=O)NC4=O)cn3)CC2)CC1]

(S)—N-(2-(6,6-dimethyl-4,5,6,7-tetrahydro-1H-indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (0.0248 g, 37.8%) was prepared by procedures analogous to Example 67 starting from 1-{6-[4-(hydroxymethyl)piperidin-1-yl]pyridin-3-yl}-1,3-diazinane-2,4-dione (28 mg, 0.09 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (40 mg, 0.09 mmol). ¹H NMR (500 MHz, DMSO-d₆) δ 11.44 (s, 1H), 10.37 (s, 1H), 8.06 (d, J=2.6 Hz, 1H), 7.61 (d, J=8.3 Hz, 1H), 7.55 (dd, J=9.1, 2.6 Hz, 1H), 7.32 (s, 1H), 6.98-6.90 (m, 2H), 6.65 (d, J=2.1 Hz, 1H), 4.28 (d, J=13.0 Hz, 2H), 3.71 (t, J=6.7 Hz, 2H), 3.60-3.32 (m, 2H), 3.25 (s, 3H), 2.91-2.82 (m, 3H), 2.75-2.65 (m, 4H), 2.51 (t, J=1.9 Hz, 11H), 2.03-1.99 (m, 1H), 1.80-1.74 (m, 2H), 1.59 (t, J=6.4 Hz, 2H), 1.18-1.15 (m, 4H), 1.02 (s, 6H). LCMS: C₄₀H₅₂N₁₀O₃ requires: 720, found: m/z=721 [M+H]⁺.

### Example 67. (2S)—N-(2-(6,6-dimethyl-4,5,6,7-tetrahydro-1H-indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (32)

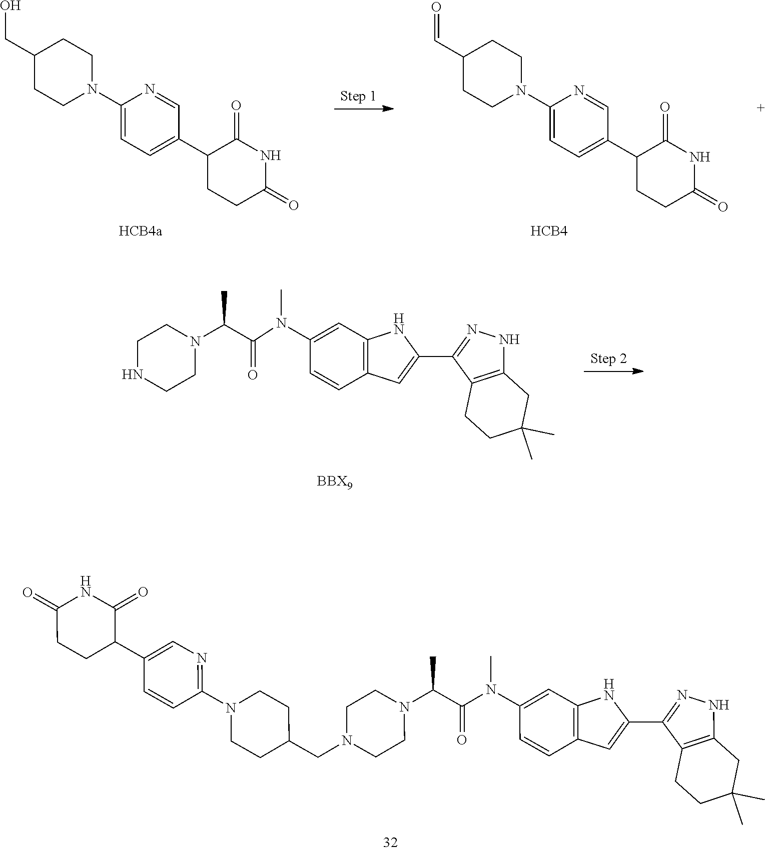

### Step 1: Synthesis of 1-(5-(2,6-dioxopiperidin-3-yl)pyridin-2-yl)piperidine-4-carbaldehyde (HCB4)

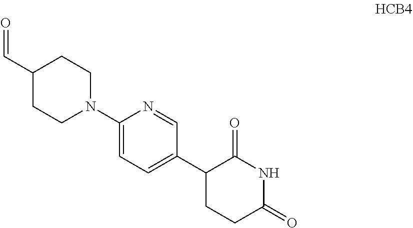 [SMILES: O=CC1CCN(c2ccc(C3CCC(=O)NC3=O)cn2)CC1]

To a mixture of 3-(6-(4-(hydroxymethyl)piperidin-1-yl)pyridin-3-yl)piperidine-2,6-dione (HCB4a) (25 mg, 0.08 mmol) in DMSO (1.00 mL) was added triethylamine (0.23 mL, 0.17 g, 1.65 mmol) followed by sulfur trioxide pyridine complex (131 mg, 0.82 mmol). After fifteen minutes, TLC (10% MeOH:DCM) indicated complete consumption of the starting material. Water was added and the mixture was extracted twice with DCM. The combined organic layers were concentrated in vacuo to provide 1-(5-(2,6-dioxopiperidin-3-yl)pyridin-2-yl)piperidine-4-carbaldehyde as a mixture containing DMSO which was used without further purification. LCMS: C₁₆H₁₉N₃O₃ requires: 301, found: m/z=302 [M+H]+.

### Step 2: Synthesis of (2S)—N-(2-(6,6-dimethyl-4,5,6,7-tetrahydro-1H-indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (32)

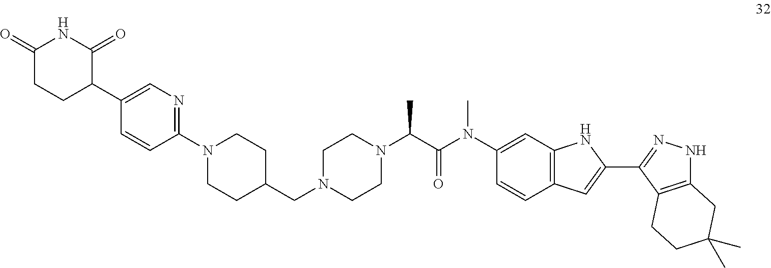 [SMILES: C[C@@H](C(=O)N(C)c1ccc2cc(-c3n[nH]c4c3CCC(C)(C)C4)[nH]c2c1)N1CCN(CC2CCN(c3ccc(C4CCC(=O)NC4=O)cn3)CC2)CC1]

To a mixture of (2S)—N-[2-(6,6-dimethyl-1,4,5,7-tetrahydroindazol-3-yl)-1H-indol-6-yl]-N-methyl-2-(piperazin-1-yl)propanamide (36 mg, 0.08 mmol) in DCM (1.00 mL) was added N,N-diisopropylethylamine (0.06 mL, 0.04 g, 0.33 mmol). The resulting mixture was added to 1-(5-(2,6-dioxopiperidin-3-yl)pyridin-2-yl)piperidine-4-carbaldehyde (25 mg, 0.08 mmol) followed by sodium triacetoxyborohydride (35 mg, 0.17 mmol). After one hour, water was added and the mixture was extracted twice with DCM. The combined organic layers were concentrated and the crude residue was purified by preparative HPLC (5 to 95% MeCN:water with 0.1% TFA) to provide (2S)—N-(2-(6,6-dimethyl-4,5,6,7-tetrahydro-1H-indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (0.0258 g, 43.3). ¹H NMR (500 MHz, DMSO-d₆) δ 11.44 (s, 1H), 10.89 (s, 1H), 9.05 (s, 1H), 7.92 (s, 1H), 7.61 (d, J=8.3 Hz, 1H), 7.33 (s, 1H), 7.12 (s, 1H), 6.95 (d, J=8.3 Hz, 1H), 6.65 (s, 1H), 4.26-4.20 (m, 2H), 3.87-3.81 (m, 1H), 3.61-3.32 (m, 3H), 3.25 (s, 3H), 3.10-2.78 (m, 7H), 2.75-2.64 (m, 3H), 2.61-2.34 (m, 11H), 2.25 (td, J=14.5, 14.0, 10.2 Hz, 1H), 2.01-1.93 (m, 1H), 1.84-1.77 (m, 2H), 1.62-1.56 (m, 2H), 1.33-1.07 (m, 4H), 1.02 (s, 6H). LCMS: C₄₁H₅₃N₉O₃ requires: 719, found: m/z=720 [M+H]⁺.

### Example 68. 1-(2-(6,6-dimethyl-4,5,6,7-tetrahydro-1H-indazol-3-yl)-1H-indole-6-carbonyl)-N-(1-(5-(2,6-dioxopiperidin-3-yl)pyridin-2-yl)piperidin-4-yl)-N-methylpiperidine-4-carboxamide (33)

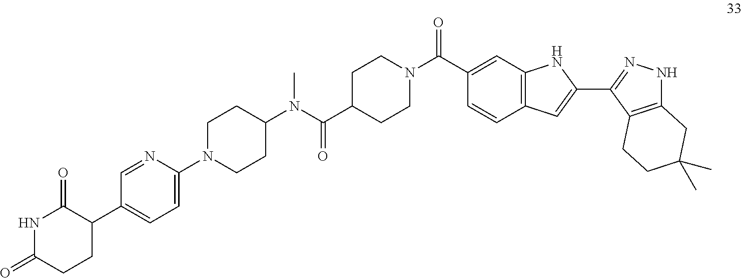 [SMILES: CN(C(=O)C1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1]

A mixture of 2-(6,6-dimethyl-4,5,6,7-tetrahydro-1H-indazol-3-yl)-1H-indole-6-carboxylic acid (74.81 mg, 0.24 mmol), DMF (1.45 mL), N,N-diisopropylethylamine (210.61 μL, 0.16 g, 1.21 mmol), and HATU (91.95 mg, 0.24 mmol) was treated with a mixture of N-(1-(5-(2,6-dioxopiperidin-3-yl)pyridin-2-yl)piperidin-4-yl)-N-methylpiperidine-4-carboxamide (100.00 mg, 0.24 mmol), N,N-diisopropylethylamine (210.61 μL, 0.16 g, 1.21 mmol), and DMF (1.45 mL). After thirty minutes, the reaction was concentrated under reduced pressure and the residue was purified by RP-HPLC (10-95% MeCN in water with 0.1% TFA) to give the desired product (0.016 g, 9.4%). ¹H NMR (500 MHz, DMSO-d₆) δ 12.57 (s, 1H), 11.43 (s, 1H), 10.81 (s, 1H), 7.94 (s, 1H), 7.53 (d, J=8.0 Hz, 1H), 7.43 (s, 1H), 7.37 (d, J=2.5 Hz, 1H), 6.98 (d, J=8.1 Hz, 1H), 6.81 (d, J=8.9 Hz, 1H), 6.61 (s, 1H), 4.33 (d, J=12.8 Hz, 2H), 3.73 (dd, J=12.1, 4.9 Hz, 1H), 2.75 (t, J=12.5 Hz, 3H), 2.71-2.65 (m, 3H), 2.43 (s, 2H), 2.27 (d, J=6.5 Hz, 2H), 2.19 (s, 3H), 2.18-2.13 (m, 1H), 2.02-1.95 (m, 1H), 1.73 (d, J=11.8 Hz, 4H), 1.59 (t, J=6.4 Hz, 2H), 1.43-1.33 (m, 2H), 1.11-1.04 (m, 2H), 1.02 (s, 6H). LCMS: C₄₀H₅₀N₈O₃ requires: 690.4, found: m/z=691.7 [M+H]⁺. LCMS: C₄₀H₄₈N₈O₄ requires: 705, found: m/z=706 [M+H]⁺.

### Example 69. 3-(4-(4-(4-(2-(6,6-dimethyl-4,5,6,7-tetrahydro-1H-indazol-3-yl)-1H-indole-6-carbonyl)piperazin-1-yl)but-1-yn-1-yl)phenyl)piperidine-2,6-dione (34)

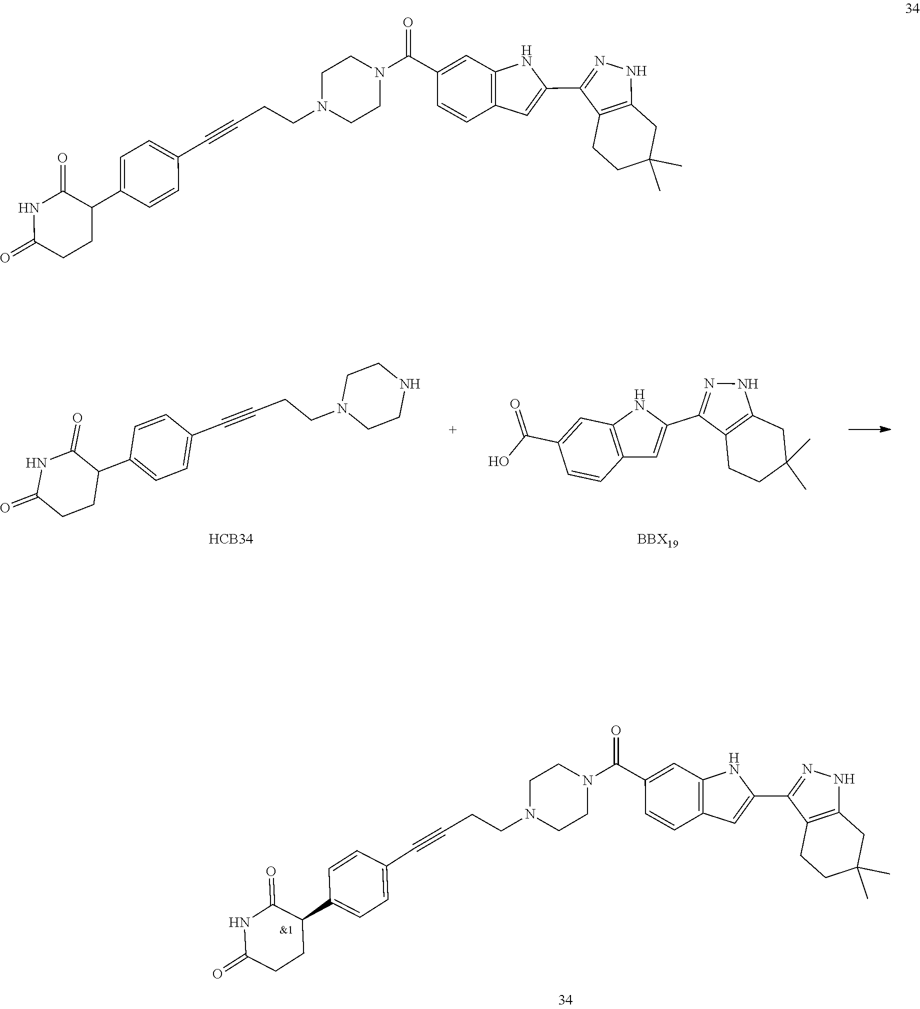

3-(4-(4-(4-(2-(6,6-dimethyl-4,5,6,7-tetrahydro-1H-indazol-3-yl)-1H-indole-6-carbonyl)piperazin-1-yl)but-1-yn-1-yl)phenyl)piperidine-2,6-dione was prepared using general procedure 1 by treating 3-(4-(4-(piperazin-1-yl)but-1-yn-1-yl)phenyl)piperidine-2,6-dione (HCB34) (30 mg, 0.09 mmol) with 2-(6,6-dimethyl-4,5,6,7-tetrahydro-1H-indazol-3-yl)-1H-indole-6-carboxylic acid (BBX₁₉) (41 mg, 0.09 mmol). Product (12 mg, 0.02 mmol, 20% yield). ¹H NMR (500 MHz, DMSO-d₆) δ 12.58 (s, 1H), 11.46 (s, 1H), 10.85 (s, 1H), 7.54 (d, J=8.1 Hz, 1H), 7.47 (s, 1H), 7.36 (d, J=7.9 Hz, 2H), 7.21 (d, J=8.0 Hz, 2H), 7.01 (d, J=8.1 Hz, 1H), 6.63 (s, 1H), 3.88 (dd, J=11.7, 4.9 Hz, 1H), 3.68-3.48 (m, 5H), 2.76-2.57 (m, 7H), 2.43 (s, 2H), 2.20 (qd, J=12.2, 4.4 Hz, 1H), 2.02 (dq, J=14.4, 5.2 Hz, 1H), 1.59 (t, J=6.4 Hz, 2H), 1.26 (q, J=6.1, 5.1 Hz, 5H), 1.02 (s, 6H). LCMS: C₃₇H₄₀N₆O₃ requires: 616.3, found: m/z=617.6 [M+H]⁺.

### Example 70. 3-(5-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-2-yl)piperidine-2,6-dione (35)

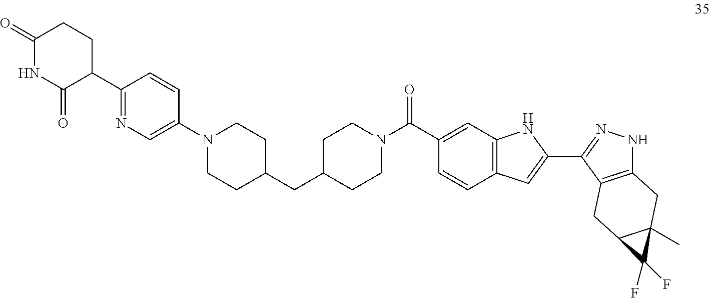 [SMILES: C[C@@]12Cc3[nH]nc(-c4cc5ccc(C(=O)N6CCC(CC7CCN(c8ccc(C9CCC(=O)NC9=O)nc8)CC7)CC6)cc5[nH]4)c3C[C@@H]1C2(F)F]

3-(5-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-2-yl)piperidine-2,6-dione was prepared using general procedure 1 by treating 3-(5-(4-(piperidin-4-ylmethyl)piperazin-1-yl)pyridin-2-yl)piperidine-2,6-dione (HCB37) (13 mg, 0.03 mmol) with 2-(6,6-dimethyl-4,5,6,7-tetrahydro-1H-indazol-3-yl)-1H-indole-6-carboxylic acid (BBX₁₉) (10.8 mg, 0.03 mmol). Product 12.2 mg, 0.02 mmol, 50% yield. ¹H NMR (500 MHz, DMSO) δ 11.46 (s, 1H), 10.87 (s, 1H), 9.37 (s, 1H), 8.33 (d, J=2.9 Hz, 1H), 7.54 (t, J=7.0 Hz, 2H), 7.45 (s, 1H), 7.34 (d, J=8.6 Hz, 1H), 7.01 (d, J=8.1 Hz, 1H), 6.63 (d, J=2.1 Hz, 1H), 4.05-3.85 (m, 3H), 3.65 (d, J=10.5 Hz, 2H), 2.77-2.58 (m, 3H), 2.43 (s, 2H), 2.33-2.01 (m, 3H), 1.81 (s, 2H), 1.59 (t, J=6.4 Hz, 2H), 1.23 (d, J=15.5 Hz, 3H), 1.02 (s, 6H). LCMS: C₃₈H₄₆N₈O₃ requires: 662.4, found: m/z=663.7 [M+H]⁺.

### Example 71. 3-(4-(1-(2-(6,6-dimethyl-4,5,6,7-tetrahydro-1H-indazol-3-yl)-1H-indole-6-carbonyl)piperidin-4-yl)phenyl)piperidine-2,6-dione (36)

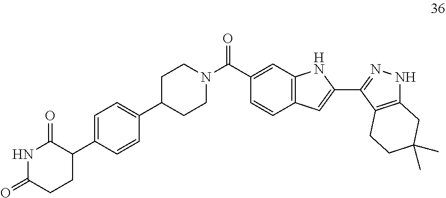 [SMILES: CC1(C)CCc2c(-c3cc4ccc(C(=O)N5CCC(c6ccc(C7CCC(=O)NC7=O)cc6)CC5)cc4[nH]3)n[nH]c2C1]

Prepared using procedure 1. A mixture of 3-(4-(piperidin-4-yl)phenyl)piperidine-2,6-dione (29 mg, 0.11 mmol), 2-(6,6-dimethyl-4,5,6,7-tetrahydro-1H-indazol-3-yl)-1H-indole-6-carboxylic acid (40 mg, 0.13 mmol), BOP (57 mg, 0.13 mmol), i-Pr₂NEt (93 μL, 0.54 mmol) in DMF (550 μL) was allowed to stir at r.t. for 16 h. The reaction mixture was purified by HPLC (H₂O/MeCN with 0.1% TFA) to afford 3-(4-(1-(2-(6,6-dimethyl-4,5,6,7-tetrahydro-1H-indazol-3-yl)-1H-indole-6-carbonyl)piperidin-4-yl)phenyl)piperidine-2,6-dione (Compound 36) (20 mg, 0.04 mmol, 33%). ¹H NMR (500 MHz, (CD₃)₂SO) δ 11.40 (s, 1H), 10.82 (s, 1H), 7.54 (d, J=8.1 Hz, 1H), 7.49 (s, 1H), 7.25 (d, J=7.9 Hz, 2H), 7.16 (d, J=7.9 Hz, 2H), 7.05 (dd, J=8.1, 1.5 Hz, 1H), 6.62 (d, J=2.1 Hz, 1H), 3.82 (dd, J=11.5, 5.0 Hz, 1H), 2.82 (tt, J=12.4, 3.6 Hz, 1H), 2.71-2.60 (m, 3H), 2.41 (s, 2H), 2.17 (ddt, J=16.0, 11.8, 5.8 Hz, 1H), 2.03 (dq, J=13.8, 4.9 Hz, 1H).

### Example 72. 3-(4-(4-((1-(2-(6,6-dimethyl-4,5,6,7-tetrahydro-1H-indazol-3-yl)-1H-indole-6-carbonyl)piperidin-4-yl)methyl)piperazin-1-yl)phenyl)piperidine-2,6-dione (37)

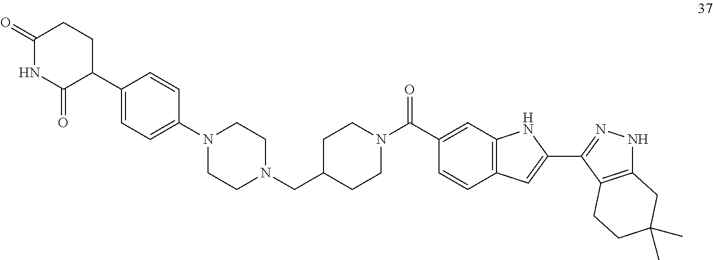 [SMILES: CC1(C)CCc2c(-c3cc4ccc(C(=O)N5CCC(CN6CCN(c7ccc(C8CCC(=O)NC8=O)cc7)CC6)CC5)cc4[nH]3)n[nH]c2C1]

3-(4-(4-((1-(2-(6,6-dimethyl-4,5,6,7-tetrahydro-1H-indazol-3-yl)-1H-indole-6-carbonyl)piperidin-4-yl)methyl)piperazin-1-yl)phenyl)piperidine-2,6-dione was prepared using general procedure 1 by treating 3-(4-(4-(piperidin-4-ylmethyl)piperazin-1-yl)phenyl)piperidine-2,6-dione (HCB35) (13 mg, 0.04 mmol) with 2-(6,6-dimethyl-4,5,6,7-tetrahydro-1H-indazol-3-yl)-1H-indole-6-carboxylic acid (BBX₁₉) (11 mg, 0.04 mmol). Product (21 mg, 0.03 mmol, 86.8% yield). ¹H NMR (500 MHz, DMSO) δ 11.46 (s, 1H), 10.80 (s, 1H), 9.29 (s, 1H), 7.55 (d, J=8.1 Hz, 1H), 7.45 (s, 1H), 7.16 (dd, J=36.2, 8.4 Hz, 2H), 6.99 (td, J=12.6, 11.3, 8.1 Hz, 3H), 6.63 (d, J=2.1 Hz, 1H), 3.88-3.73 (m, 3H), 3.63 (d, J=11.2 Hz, 3H), 3.58 (s, 1H), 3.17 (d, J=19.4 Hz, 4H), 3.10-2.92 (m, 4H), 2.68 (q, J=8.0, 7.2 Hz, 2H), 2.43 (s, 2H), 2.29-1.93 (m, 4H), 1.94-1.71 (m, 2H), 1.59 (t, J=6.4 Hz, 2H), 1.23 (d, J=13.6 Hz, 3H), 1.02 (s, 6H). LCMS: C₃₉H₄₇N₇O₃ requires: 661.4, found: m/z=662.7 [M+H]⁺.

### Example 73. 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)phenyl)piperidine-2,6-dione (38)

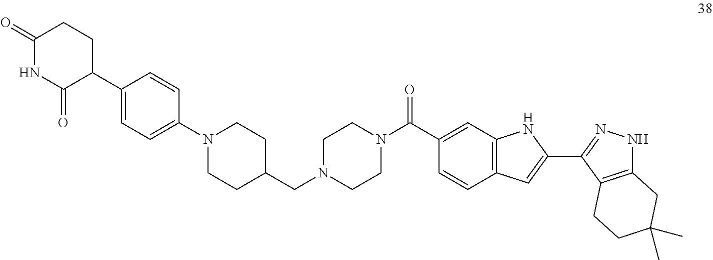 [SMILES: CC1(C)CCc2c(-c3cc4ccc(C(=O)N5CCN(CC6CCN(c7ccc(C8CCC(=O)NC8=O)cc7)CC6)CC5)cc4[nH]3)n[nH]c2C1]

3-{4-[4-((4-[2-(6,6-dimethyl-1,4,5,7-tetrahydroindazol-3-yl)-1H-indole-6-carbonyl]piperazin-1-yl}methyl)piperidin-1-yl]phenyl)piperidine-2,6-dione was prepared using general procedure 1 by treating 3-(4-(4-(piperazin-1-ylmethyl)piperidin-1-yl)phenyl)piperidine-2,6-dione (HCB36) (73.4 mg, 0.2 mmol) with 2-(6,6-dimethyl-4,5,6,7-tetrahydro-1H-indazol-3-yl)-1H-indole-6-carboxylic acid (BBX₁₉) (61.3 mg, 0.2 mmol). Product (51 mg, 0.08 mmol, 38% yield). ¹H NMR (500 MHz, DMSO) δ 11.44 (s, 1H), 10.77 (s, 1H), 7.54 (d, J=8.1 Hz, 1H), 7.46 (s, 1H), 7.03 (t, J=10.5 Hz, 3H), 6.89 (d, J=8.3 Hz, 2H), 6.68-6.57 (m, 1H), 3.72 (dd, J=10.9, 4.9 Hz, 1H), 3.69-3.42 (m, 7H), 2.75-2.56 (m, 5H), 2.41 (d, J=15.0 Hz, 6H), 2.28-2.07 (m, 3H), 2.02 (dt, J=13.2, 5.0 Hz, 1H), 1.81 (d, J=12.6 Hz, 2H), 1.68 (s, 1H), 1.59 (t, J=6.4 Hz, 2H), 1.22 (dd, J=21.6, 10.8 Hz, 3H), 1.02 (s, 7H). LCMS: C₃₉H₄₇N₇O₃ requires: 661.4, found: m/z=662.7 [M+H]⁺.

### Example 74. 3-(4-(1-((1-(2-(6,6-dimethyl-4,5,6,7-tetrahydro-1H-indazol-3-yl)-1H-indole-6-carbonyl)piperidin-4-yl)methyl)piperidin-4-yl)phenyl)piperidine-2,6-dione (39)

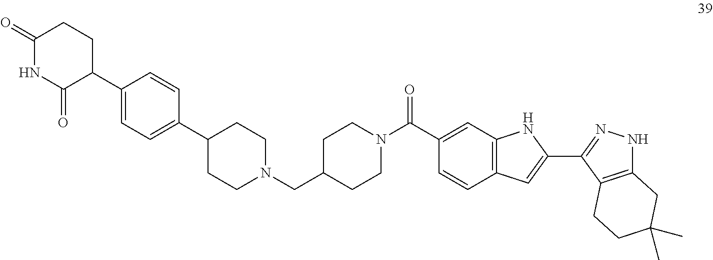 [SMILES: CC1(C)CCc2c(-c3cc4ccc(C(=O)N5CCC(CN6CCC(c7ccc(C8CCC(=O)NC8=O)cc7)CC6)CC5)cc4[nH]3)n[nH]c2C1]

3-(4-(1-((1-(2-(6,6-dimethyl-4,5,6,7-tetrahydro-1H-indazol-3-yl)-1H-indole-6-carbonyl)piperidin-4-yl)methyl)piperidin-4-yl)phenyl)piperidine-2,6-dione was prepared using general procedure 1 by treating 3-(4-(1-(piperidin-4-ylmethyl)piperidin-4-yl)phenyl)piperidine-2,6-dione (HCB33) (17 mg, 0.05 mmol) with 2-(6,6-dimethyl-4,5,6,7-tetrahydro-1H-indazol-3-yl)-1H-indole-6-carboxylic acid (BBX₁₉) (14 mg, 0.05 mmol). Product (19.8 mg, 0.03 mmol, 66% yield). ¹H NMR (500 MHz, DMSO-d₆) δ 11.47 (s, 1H), 10.84 (s, 1H), 7.55 (d, J=8.1 Hz, 1H), 7.45 (s, 1H), 7.28-7.15 (m, 4H), 7.01 (d, J=8.0 Hz, 1H), 6.63 (d, J=2.1 Hz, 1H), 3.84 (dd, J=11.6, 5.0 Hz, 1H), 3.63 (d, J=11.8 Hz, 2H), 3.13-3.01 (m, 4H), 2.86-2.76 (m, 1H), 2.67 (dt, J=17.0, 5.3 Hz, 3H), 2.43 (s, 2H), 2.25-2.12 (m, 2H), 2.09-1.94 (m, 5H), 1.85 (s, 2H), 1.59 (t, J=6.4 Hz, 2H), 1.33-1.12 (m, 2H), 1.02 (s, 6H). LCMS: C₄₀H₄₈N₆O₃ requires: 660.4, found: m/z=661.7 [M+H]⁺.

### Example 75. 3-(4-(4-(4-(2-(6,6-dimethyl-4,5,6,7-tetrahydro-1H-indazol-3-yl)-1H-indole-6-carbonyl)piperazin-1-yl)butyl)phenyl)piperidine-2,6-dione (40)

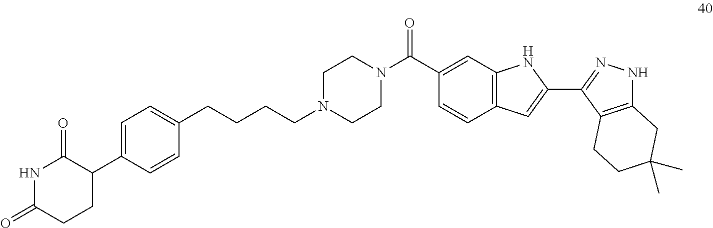 [SMILES: CC1(C)CCc2c(-c3cc4ccc(C(=O)N5CCN(CCCCc6ccc(C7CCC(=O)NC7=O)cc6)CC5)cc4[nH]3)n[nH]c2C1]

A suspension of rac-(R)-3-[4-(4-{4-[2-(6,6-dimethyl-1,4,5,7-tetrahydroindazol-3-yl)-1H-indole-6-carbonyl]piperazin-1-yl}but-1-yn-1-yl)phenyl]piperidine-2,6-dione (34) (10.00 mg, 0.02 mmol) and 10 mg Pd on carbon in EtOH (2 ml) was vigorously stirred under H₂ atmosphere (balloon) for 16 hours. The solids were filtered off, washed with DCM and the solution was evaporated to crude product. The crude product was purified by TLC developed by 5% MeOH in EtOAc, obtained rac-(R)-3-[4-(4-{4-[2-(6,6-dimethyl-1,4,5,7-tetrahydroindazol-3-yl)-1H-indole-6-carbonyl]piperazin-1-yl}butyl)phenyl]piperidine-2,6-dione (3 mg, 28%). ¹H NMR (500 MHz, Acetonitrile-d₃) δ 9.94 (d, J=51.6 Hz, 1H), 9.42 (s, 1H), 7.62 (d, J=8.0 Hz, 1H), 7.46 (s, 1H), 7.27-7.15 (m, 3H), 7.10 (d, J=8.2 Hz, 1H), 6.74 (d, J=2.2 Hz, 1H), 3.79 (dd, J=11.5, 5.1 Hz, 1H), 3.62 (s, 6H), 2.78 (t, J=6.3 Hz, 2H), 2.66 (dq, J=8.1, 5.3, 4.8 Hz, 3H), 2.49 (s, 5H), 1.67 (t, J=6.3 Hz, 4H), 1.59-1.47 (m, 2H), 1.41-1.24 (m, 3H), 1.07 (s, 6H). LCMS: C₃₇H₄₄N₆O₃ requires: 620.3, found: m/z=621.7 [M+H]⁺.

### Example 76. 3-(5-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-2-yl)piperidine-2,6-dione (41)

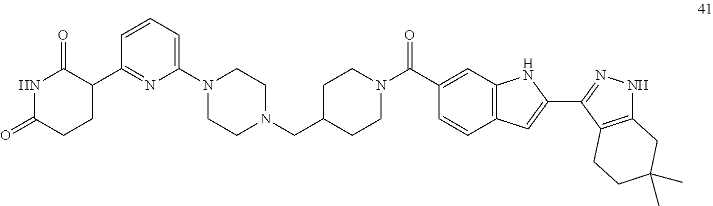 [SMILES: CC1(C)CCc2c(-c3cc4ccc(C(=O)N5CCC(CN6CCN(c7cccc(C8CCC(=O)NC8=O)n7)CC6)CC5)cc4[nH]3)n[nH]c2C1]

3-(5-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-2-yl)piperidine-2,6-dione was prepared using general procedure 1 by treating 3-(6-(4-(piperidin-4-ylmethyl)piperazin-1-yl)pyridin-2-yl)piperidine-2,6-dione (HCB39) (13 mg, 0.03 mmol) with 2-(6,6-dimethyl-4,5,6,7-tetrahydro-1H-indazol-3-yl)-1H-indole-6-carboxylic acid (BBX₁₉) (10.8 mg, 0.03 mmol). Product (16.1 mg, 0.02 mmol, 68% yield). ¹H NMR (500 MHz, DMSO) δ 11.46 (s, 1H), 10.83 (s, 1H), 9.40 (s, 1H), 7.62 (t, J=7.9 Hz, 1H), 7.55 (d, J=8.1 Hz, 1H), 7.45 (s, 1H), 7.01 (dd, J=8.1, 1.4 Hz, 1H), 6.87 (d, J=8.5 Hz, 1H), 6.74 (d, J=7.3 Hz, 1H), 6.63 (d, J=2.1 Hz, 1H), 4.31 (d, J=13.7 Hz, 2H), 3.88 (dd, J=8.1, 5.4 Hz, 1H), 3.64-3.55 (m, 2H), 3.19 (dd, J=12.6, 6.0 Hz, 1H), 3.15-3.02 (m, 3H), 2.69 (t, J=6.5 Hz, 2H), 2.57 (ddd, J=9.2, 7.4, 5.2 Hz, 2H), 2.43 (s, 2H), 2.18 (dtd, J=27.7, 13.3, 11.9, 6.1 Hz, 3H), 1.80 (s, 2H), 1.59 (t, J=6.4 Hz, 2H), 1.22 (d, J=11.3 Hz, 2H), 1.02 (s, 6H). LCMS: C₃₈H₄₆N₈O₃ requires: 662.4, found: m/z=663.8 [M+H]⁺.

### Example 77. 3-(5-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-2-yl)piperidine-2,6-dione (42)

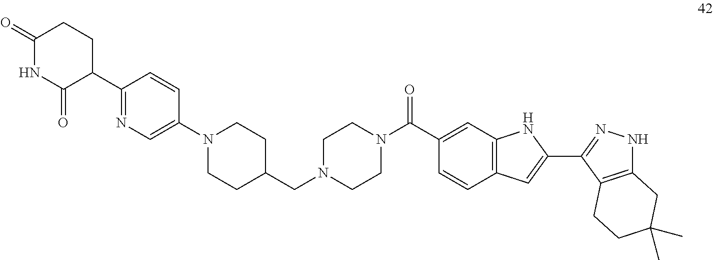 [SMILES: CC1(C)CCc2c(-c3cc4ccc(C(=O)N5CCN(CC6CCN(c7ccc(C8CCC(=O)NC8=O)nc7)CC6)CC5)cc4[nH]3)n[nH]c2C1]

3-(5-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-2-yl)piperidine-2,6-dione was prepared using general procedure 1 by treating 3-(5-(4-(piperazin-1-ylmethyl)piperidin-1-yl)pyridin-2-yl)piperidine-2,6-dione (HCB38) (13 mg, 0.03 mmol) with 2-(6,6-dimethyl-4,5,6,7-tetrahydro-1H-indazol-3-yl)-1H-indole-6-carboxylic acid (BBX₁₉) (10.8 mg, 0.03 mmol). Product (4.5 mg, 0.01 mmol, 18.4% yield). ¹H NMR (500 MHz, DMSO-d₆) δ 12.60 (s, 1H), 11.51 (d, J=50.7 Hz, 1H), 10.79 (s, 1H), 9.51 (s, 1H), 8.35-8.06 (m, 1H), 7.61-7.51 (m, 2H), 7.35 (d, J=8.9 Hz, 1H), 7.19 (d, J=8.7 Hz, 1H), 7.10 (d, J=8.2 Hz, 1H), 6.65 (s, 1H), 3.89 (dd, J=8.9, 5.2 Hz, 1H), 3.79 (d, J=12.2 Hz, 2H), 3.49 (d, J=60.7 Hz, 3H), 3.21-3.04 (m, 4H), 2.81-2.61 (m, 5H), 2.26-2.15 (m, 1H), 2.15-2.06 (m, 1H), 2.06-1.92 (m, 1H), 1.83 (s, 2H), 1.59 (t, J=6.4 Hz, 2H), 1.34 (d, J=11.6 Hz, 1H), 1.19 (t, J=7.3 Hz, 1H), 1.02 (s, 6H). LCMS: C₃₈H₄₆N₈O₃ requires: 662.4, found: m/z=663.5 [M+H]⁺.

### Example 78: Harness Aldehyde Syntheses

### Harness 1: 1-(5-(2,6-dioxopiperidin-3-yl)pyridin-3-yl)piperidine-4-carbaldehyde (Har01)

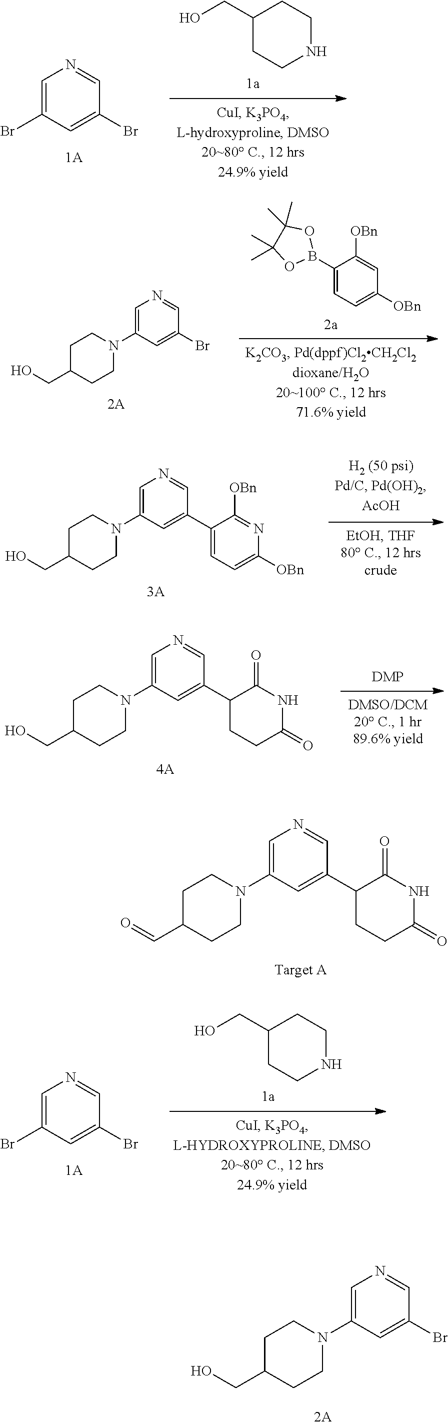

To a solution of Cpd_1A (75.0 g, 316 mmol, 1.00 eq) and Cpd_1a (36.5 g, 316 mmol, 1.00 eq) in DMSO (600 mL) was added L-HYDROXYPROLINE (8.30 g, 63.3 mmol, 0.200 eq), CuI (12.1 g, 63.3 mmol, 0.200 eq) and K₃PO₄ (134 g, 633 mmol, 2.00 eq) at 20° C. under N₂. The mixture was stirred at 80° C. for 12 hrs under N₂. LCMS showed that Cpd_1A was consumed and the desired mass (RT=0.510 min) was detected. The mixture was cooled to 20° C. and poured into water (1000 mL). The mixture was stirred at 20° C. for 0.5 hr. The mixture was extracted with ethyl acetate (1500 mL*3). The organic layers were washed with brine (3000 mL), dried over Na₂SO₄, filtered and concentrated. The residue was purified by column chromatography (SiO₂, Petroleum ether/Ethyl acetate=5/1 to 0/1, R_{f}=0.35, Petroleum ether/Ethyl acetate=0/1). Cpd_2A (22.0 g, 78.9 mmol, 24.9% yield, 97.3% purity) was obtained as yellow solid. LCMS:RT=0.510 min, m/z=271.0 (M+H)⁺; ¹H NMR: (400 MHz, CDCl₃) δ 8.20 (d, J=2.8 Hz, 1H), 8.06 (d, J=1.6 Hz, 1H), 7.30 (t, J=2.0 Hz, 1H), 3.73 (d, J=12.8 Hz, 2H), 3.73 (d, J=6.4 Hz, 2H), 2.84-2.77 (m, 2H), 1.87 (d, J=12.8 Hz, 3H), 1.75-1.60 (m, 1H), 1.42-1.33 (m, 2H)

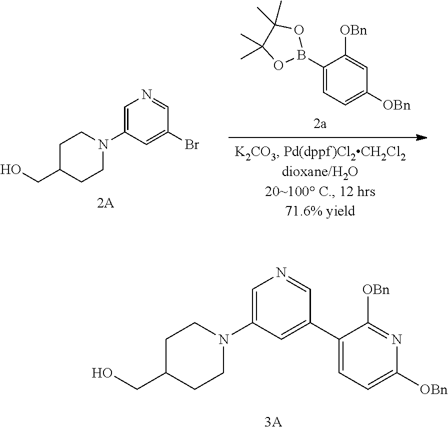 [SMILES: OCC1CCN(c2cncc(Br)c2)CC1 ; CC1(C)OB(c2ccc(OCc3ccccc3)cc2OCc2ccccc2)OC1(C)C ; OCC1CCN(c2cncc(-c3ccc(OCc4ccccc4)nc3OCc3ccccc3)c2)CC1]

To a solution of Cpd_2A (21.0 g, 77.4 mmol, 1.00 eq) and Cpd_2a (32.3 g, 77.4 mmol, 1.00 eq) in dioxane (263 mL) and H₂O (53.0 mL) was added K₂CO₃ (32.1 g, 232 mmol, 3.00 eq) and Pd(dppf)Cl₂CH₂Cl₂ (6.32 g, 7.74 mmol, 0.100 eq) at 20° C. under N₂. The mixture was degassed and then heated to 100° C. for 12 hrs under N₂. LCMS showed that Cpd_2A was consumed and the desired mass (RT=0.829 min) was detected. The reaction was cooled to 20° C. and concentrated. The residue was poured into water (500 mL) and extracted with ethyl acetate (500 mL*3). The combined organic layers were washed with brine (1000 mL), dried over Na₂SO₄, filtered and concentrated. The residue was purified by column chromatography (SiO₂, Petroleum ether/ethyl acetate=50/1-25/1-3/1). The product was given (Petroleum ether/Ethyl acetate=0/1, R_{f}=0.20). Cpd_3A (30.3 g, 60.4 mmol, 71.6% yield, 95.8% purity) was obtained as a brown oil. LCMS: EW34522-9-P1A1, product: RT=0.829 min, m/z=482.2 (M+H)⁺; ¹H NMR: (400 MHz, DMSO d₆) δ 8.22-8.06 (m, 2H), 7.82 (d, J=8.0 Hz, 1H), 7.49-7.29 (m, 11H), 6.57 (d, J=8.2 Hz, 1H), 5.39 (d, J=14.4 Hz, 4H), 4.52 (t, J=5.4 Hz, 1H), 3.66 (br d, J=12.2 Hz, 2H), 3.28 (t, J=5.8 Hz, 2H), 2.69-2.54 (m, 2H), 1.70 (br d, J=11.4 Hz, 2H), 1.57-1.42 (m, 1H), 1.24-1.17 (m, 2H)

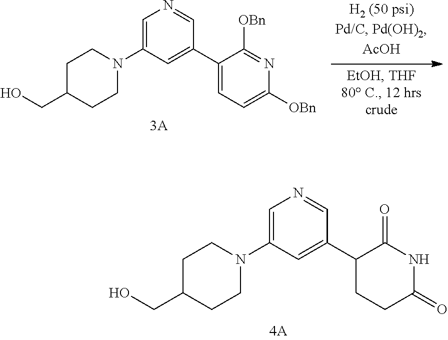 [SMILES: OCC1CCN(c2cncc(-c3ccc(OCc4ccccc4)nc3OCc3ccccc3)c2)CC1 ; O=C1CCC(c2cncc(N3CCC(CO)CC3)c2)C(=O)N1]

To a solution of Cpd_3A (28.0 g, 55.7 mmol, 95.8% purity, 1.00 eq) and AcOH (10.0 g, 167 mmol, 9.56 mL, 3.00 eq) in EtOH (140 mL) and THF (140 mL) was added Pd/C (7.00 g, 55.7 mmol, 10.0% purity, 1.00 eq) and Pd(OH)₂ (7.00 g, 9.97 mmol, 20.0% purity, 0.179 eq) under N₂. The suspension was degassed under vacuum and purged with H₂ for 3 times. The reaction mixture was stirred under H₂ (50.0 psi) at 80° C. for 12 hours. LCMS showed that Cpd_3A (28.0 g, 55.7 mmol, 95.8% purity, 1.00 eq) consumed and the desired mass (RT=0.672 min) was given. The suspension was filtered through a pad of Celite and the pad was washed with EtOH (200 mL*4). Evaporated the solution on a water bath under reduced pressure using a rotary evaporator. Cpd_4A (20.0 g, crude) was obtained as a gray solid. LCMS: product: RT=0.672 min, m/z=304.0 (M+H)⁺; ¹H NMR: (400 MHz, DMSO) δ 8.18 (br d, J=2.0 Hz, 1H), 7.82 (s, 1H), 7.20 (br s, 1H), 3.83 (br dd, J=4.8, 12.0 Hz, 1H), 3.76 (br d, J=11.0 Hz, 2H), 3.31-3.22 (m, 2H), 2.70 (br s, 2H), 2.53 (br d, J=4.0 Hz, 1H), 2.29 (dt, J=8.8, 12.6 Hz, 1H), 2.05-1.95 (m, 1H), 1.74 (br d, J=12.4 Hz, 3H), 1.54 (br d, J=2.8 Hz, 2H), 1.25-1.17 (m, 2H)

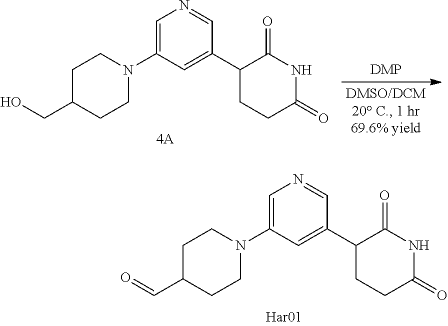 [SMILES: O=C1CCC(c2cncc(N3CCC(CO)CC3)c2)C(=O)N1 ; O=CC1CCN(c2cncc(C3CCC(=O)NC3=O)c2)CC1]

To a solution of Cpd_4A (5.00 g, 14.9 mmol, 90.5% purity, 1.00 eq) in DMSO (25.0 mL) and DCM (100 mL) was added DMP (12.6 g, 29.8 mmol, 9.24 mL, 2.00 eq) slowly at 20° C. The reaction was stirred at 20° C. for 1 hr. LCMS showed that Cpd_4A consumed and the desired mass (RT=0.659 min) was given. The mixture was poured into water (200 mL). The mixture was adjusted with saturated aqueous Na₂CO₃ until pH=10 and the aqueous layer was extracted with DCM (500 mL*6). The combined organic layers were washed with Na₂S₂O₃ solution (200 mL*2), dried over Na₂SO₄, concentrated under vacuum. The residue was treated with ethyl acetate (20.0 mL) at 25° C. for 12 hrs, then filtered, and the filter cake was concentrated vacuum. Har01 (3.39 g, 10.3 mmol, 69.6% yield, 92.3% purity) was obtained as a light brown solid. LCMS: product: RT=0.857 min, m/z=300.1 (M+H)¹H NMR: (400 MHz, DMSO_d₆) δ 10.86 (s, 1H), 9.63 (s, 1H), 8.19 (d, J=2.8 Hz, 1H), 7.84 (d, J=1.6 Hz, 1H), 7.21 (br s, 1H), 3.83 (dd, J=4.8, 12.4 Hz, 1H), 3.79-3.60 (m, 2H), 2.87 (br t, J=10.4 Hz, 1H), 2.74-2.60 (m, 2H), 2.54 (br s, 2H), 2.33-2.27 (m, 1H), 2.02-1.90 (m, 3H), 1.66-1.47 (m, 2H)

### Example 79: Harness 2: 1-(4-(2,6-dioxopiperidin-3-yl)pyridin-2-yl)piperidine-4-carbaldehyde (Har02)

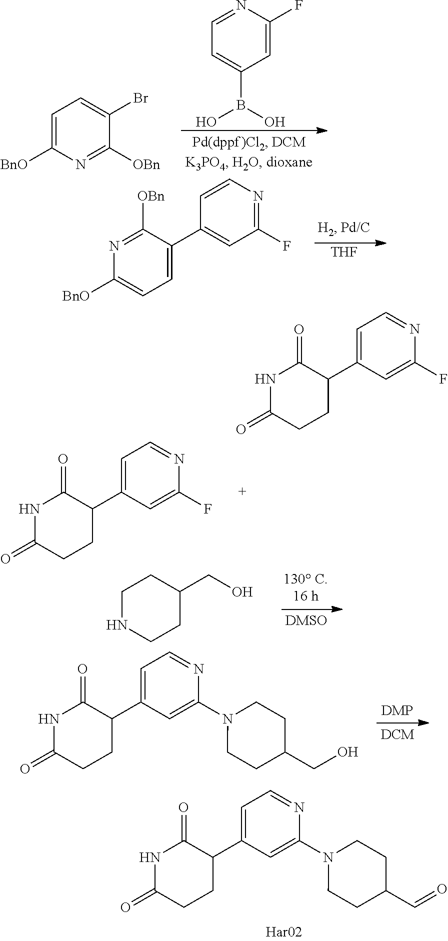

### Step 1: 3-(2-fluoropyridin-4-yl)piperidine-2,6-dione

Into a 5-L 4-necked round-bottom flask purged and maintained with an inert atmosphere of nitrogen, was placed 2,6-bis(benzyloxy)-3-bromopyridine (408 g, 1101.97 mmol, 1.00 equiv), dioxane (4080 mL), water (816 mL), K₃PO₄ (701.73 g, 3305.91 mmol, 3.00 equiv), 2-fluoropyridin-4-ylboronic acid (163.04 g, 1157.06 mmol, 1.05 equiv), Pd(dppf)Cl₂ (40.32 g, 55.09 mmol, 0.05 equiv). The resulting solution was stirred for 3 h at 100° C. The mixture was allowed to cool down to RT. The solids were filtered out. The resulting mixture was concentrated. The residue was applied onto a silica gel column with ethyl acetate/petroleum ether (8%). This resulted in 2,6-bis(benzyloxy)-2′-fluoro-3,4′-bipyridine as a white solid (310 g, 72.8%). LCMS: C24H19FN2O2 requires: 386, found: m/z=387 [M+H]⁺.

### Step 2: 2,6-bis(benzyloxy)-2′-fluoro-3,4′-bipyridine

Into a 10-L 4-necked round-bottom flask, was placed 2,6-bis(benzyloxy)-2′-fluoro-3,4′-bipyridine (340 g, 879.85 mmol, 1.00 equiv), THF (6800 mL), Pd/C (34 g, 319.48 mmol, 0.36 equiv). To the above H₂ (gas) was introduced to keep pressure about 3 atm. The resulting solution was stirred for overnight at room temperature. After 16 h, additional Pd/C (10 g, 93.96 mmol, 0.11 equiv) was added, the resulting solution was stirred 30 h under the same condition as before. Then the solids were filtered out. The resulting mixture was concentrated. This resulted in 3-(2-fluoropyridin-4-yl)piperidine-2,6-dione as a pale yellow solid (103 g, 56.0%). LCMS: C10H9FN2O2 requires: 208, found: m/z=209 [M+H]⁺.

### Step 3: 3-(2-(4-(hydroxymethyl)piperidin-1-yl)pyridin-4-yl)piperidine-2,6-dione

To a 40 mL vial was added 3-(2-fluoropyridin-4-yl)piperidine-2,6-dione (1000.00 mg, 4.80 mmol), piperidin-4-ylmethanol (580.88 mg, 5.04 mmol), N,N-diisopropylethylamine (3.36 mL, 2.48 g, 19.21 mmol), and DMSO (7.00 mL). The reaction mixture was stirred at 120 C for 16 h. The reaction mixture was then concentrated and purified by RP-FC (415 g C18 silica, 5-20% MeCN/H2O+0.1% TFA) to yield the title compound as a white solid (1.73 g, quantitative yield). LCMS. C₁₆H₂₁N₃O₃ requires: 303.2, found: m/z=304.5 [M+H]⁺.

### Step 4: Synthesis of 1-(4-(2,6-dioxopiperidin-3-yl)pyridin-2-yl)piperidine-4-carbaldehyde

To a 200 mL flask was added 3-{2-[4-(hydroxymethyl)piperidin-1-yl]pyridin-4-yl}piperidine-2,6-dione; trifluoroacetic acid (1710.00 mg, 4.10 mmol), DCM (50.00 mL), and MeCN (10.00 mL). The reaction mixture was cooled to 0 C, and 1,1-bis(acetyloxy)-3-oxo-1lambda5,2-benziodaoxol-1-yl acetate (1.91 g, 4.51 mmol) was added in one portion. After stirring at 0 C for 10 min, the reaction mixture was stirred warming to RT for 3 h. 3 mL TEA was added to the reaction mixture, then the crude mixture was adsorbed onto silica, then purified by column chromatography (120 g silica, 10-100% EtOAc/DCM) to yield the title compound (Har02) as a white solid (233 mg, 18%). LCMS C₁₆H₁₉N₃O₃ requires: 301.1, found: m/z=302.1 [M+H]⁺.

### Example 80: Harness 3: 1-(5-(2,6-dioxopiperidin-3-yl)pyridin-2-yl)piperidine-4-carbaldehyde (Har03)

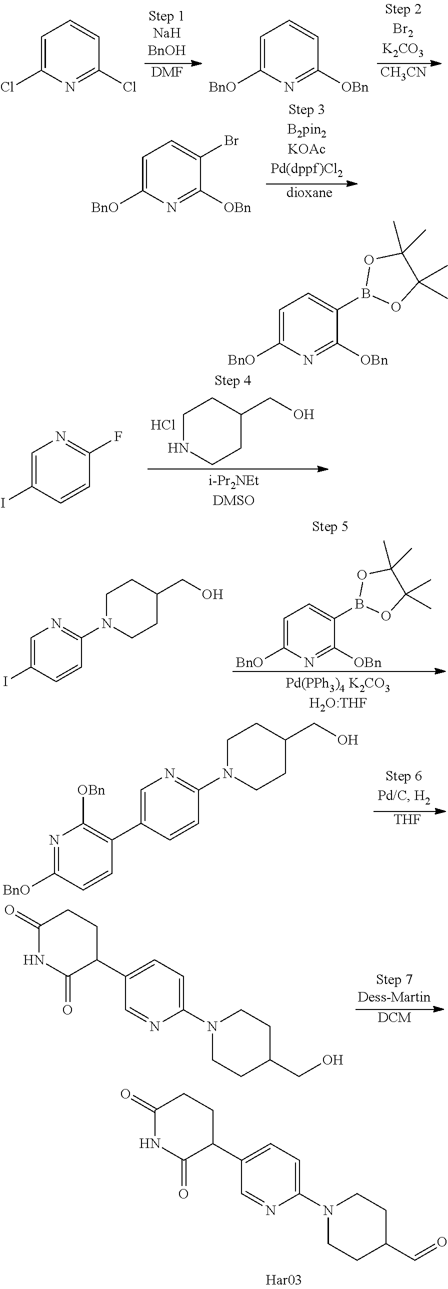

Step 1: Into a 5-L 4-necked round-bottom flask under an inert atmosphere of nitrogen, was added 2,6-dichloropyridine (150.00 g, 1013.58 mmol, 1.00 eq), dimethylformamide (3 L), NaH (272.00 g, 11334 mmol, 11.18 eq, 65%). This was followed by the addition of BnOH (329.50 g, 3050 mmol, 3.01 eq) dropwise with stirring at 0° C. The resulting solution was stirred for 4 h at 80° C. The reaction mixture was cooled. The reaction was then quenched by the addition of 7 L of water/ice. The solids were collected by filtration and concentrated. This resulted in 276 g (93.46%) of 2,6-bis(benzyloxy)pyridine as a grey solid. LCMS: (ES, m/z): [M+1]⁺=292; T=1.48 min.

Step 2: Into a 3-L 4-necked round-bottom under an inert atmosphere of nitrogen, was added 2,6-bis(benzyloxy)pyridine (276.00 g, 947.314 mmol, 1.00 eq), CH₃CN (2.76 L), K₂CO₃ (445.00 g, 3196.54 mmol, 3.37 eq). This was followed by the dropwise addition of Br₂ (151.70 g, 949.26 mmol, 1.00 eq) at 0° C. The resulting solution was stirred for 4 h at room temperature. The resulting reaction mixture was concentrated. The residue was loaded onto a silica gel column with ethyl acetate/petroleum ether (10%). This resulted in 253 g (72.13%) of 2,6-bis(benzyloxy)-3-bromopyridine as a white solid. LCMS: [M+1]⁺=370.

Step 3: Into a 3-L 4-necked round-bottom flask purged and maintained with an inert atmosphere of nitrogen, was placed 2,6-bis(benzyloxy)-3-bromopyridine (253.00 g, 683.32 mmol, 1.00 eq), Dioxane (2.53 L), bis(pinacolato)diboron (261.00 g, 1027.80 mmol, 1.50 eq), potassium acetate (134.00 g, 1365.36 mmol, 2.00 eq), Pd(dppf)Cl₂ (25.10 g, 34.290 mmol, 0.05 eq). The resulting solution was stirred for overnight at 100° C. The reaction mixture was cooled. The resulting mixture was concentrated and loaded onto a silica gel column with ethyl acetate/petroleum ether (15%). This resulted in 200 g (70.14%) of 2,6-bis(benzyloxy)-3-(4,4,5,5-tetramethyl-1,3,2-dioxaborolan-2-yl)pyridine as a white solid.

Step 4: Into a 3-L 4-necked round-bottom flask purged and maintained with an inert atmosphere of nitrogen, was placed 2-fluoro-5-iodopyridine (200.00 g, 896.90 mmol, 1.00 eq), DMSO (2.00 L), piperidin-4-ylmethanol (128.90 g, 1119.15 mmol, 1.25 eq), DIEA (347.00 g, 2684.86 mmol, 3.00 eq). The resulting solution was stirred for 3 days at 90° C. The reaction mixture was cooled. The resulting mixture was exacted with 2×2 L of EA and the organic layer was combined. The resulting mixture was washed with 3×2 L of brine. The resulting mixture was concentrated. The residue was applied onto a silica gel column with ethyl acetate/petroleum ether (35%). This resulted in 182 g (63.78%) of [1-(5-iodopyridin-2-yl)piperidin-4-yl]methanol as yellow oil. LCMS: [M+1]⁺=319

Step 5: Into a 3-L 4-necked round-bottom flask purged and maintained with an inert atmosphere of nitrogen, was placed [1-(5-iodopyridin-2-yl)piperidin-4-yl]methanol (182.00 g, 572.04 mmol, 1.00 eq), tetrahydrofuran (1.82 L), water (364.00 mL), 2,6-bis(benzyloxy)-3-(4,4,5,5-tetramethyl-1,3,2-dioxaborolan-2-yl)pyridine (298.40 g, 715.05 mmol, 1.25 eq), K₂CO₃ (157.90 g, 1134.23 mmol, 1.98 equiv), tetrakis(triphenylphosphine)palladium(0) (66.00 g, 57.114 mmol, 0.10 eq). The resulting solution was stirred for overnight at 90° C. The reaction mixture was cooled. The resulting mixture was exacted with 2×3 L of EA and the organic layer was washed with 3×3 L of brine. The combined organic layers were concentrated. The residue was loaded onto a silica gel column with ethyl acetate/petroleum ether (85%). This resulted in 140 g (50.82%) of [1-[2′,6′-bis(benzyloxy)-[3,3′-bipyridin]-6-yl]piperidin-4-yl]methanol as a green solid. LCMS: [M+1]⁺=482.

Step 6: Into a 2-L round-bottom flask, was added [1-[2′,6′-bis(benzyloxy)-[3,3′-bipyridin]-6-yl]piperidin-4-yl]methanol (35.00 g, 72.67 mmol, 1.00 eq), tetrahydrofuran (120 mL), Pd/C (10.00 g, 10%). The resulting solution was stirred overnight under hydrogen atmosphere (4 atm). The Pd/C was then filtered, followed by the addition of another 10 g of Pd/C(10%), then stirred overnight again. The filtration/addition sequence was repeated 3 times. The solids were filtered. The resulting filtrate was concentrated and washed with 3×100 mL EA, This resulted in 64 g crude product. The 8 g crude directly used into the next step and the remaining 56 g crude was further purified by flash chromatography. This resulted in 45 g of 6′-[4-(hydroxymethyl)piperidin-1-yl]-1,3-dihydro-[3,3′-bipyridine]-2,6-dione as a yellow solid. LCMS: [M+1]⁺=304. ¹H-NMR: (300 MHZ, DMSO-d₆, ppm): δ10.79 (s, 1H), 7.93 (d, J=2.3 Hz, 1H), 7.35 (dd, J=8.8, 2.5 Hz, 1H), 6.79 (d, J=8.8 Hz, 1H), 4.45 (t, J=5.1 Hz, 1H), 4.27 (d, J=12.9 Hz, 2H), 3.72 (dd, J=12.0, 4.9 Hz, 1H), 3.27 (t, J=5.1 Hz, 2H), 2.82-2.59 (m, 3H), 2.58-2.43 (m, 1H), 2.17 (qd, J=12.5, 4.4 Hz, 1H), 1.98 (dq, J=8.5, 4.7 Hz, 1H), 1.71 (d, J=13.3 Hz, 2H), 1.60 (br, 1H), 1.11 (qd, J=11.9, 3.8 Hz, 2H).

Step 7: Into a 1 L 3-necked round-bottom flask under an atmosphere of nitrogen, was added 3-[6-[4-(hydroxymethyl)piperidin-1-yl]pyridin-3-yl]piperidine-2,6-dione (8.00 g, 26.37 mmol, 1.00 eq), DCM (400.00 mL). This was followed by the addition of Dess-Martin periodinane (12.30 g, 31.59 mmol, 1.20 eq) at 0° C. The resulting solution was stirred for 2 h at 0° C. The reaction mixture was filtered, and the filtrate was washed with brine (200 mL), dried over Na₂SO₄ and concentrated in vacuum and purified by silica gel column with DCM/EA (3:2). The residue was loaded onto a silica gel column with ethyl acetate/petroleum ether (15%). This resulted in 5 g (62.92%) of 1-[5-(2,6-dioxopiperidin-3-yl)pyridin-2-yl]piperidine-4-carbaldehyde (Har03) as a grey solid. LCMS: (ES, m/z): [M+1]⁺=302. ¹H-NMR: (300 MHZ, DMSO-d₆, ppm): δ10.80 (s, 1H), 9.62 (s, 1H), 7.95 (d, J=2.5 Hz, 1H), 7.39 (dd, J=8.9, 2.5 Hz, 1H), 6.83 (d, J=8.8 Hz, 1H), 4.12 (dd, J=13.1, 4.2 Hz, 2H), 3.73 (dd, J=12.0, 4.9 Hz, 1H), 3.10-2.95 (m, 2H), 2.64 (tdd, J=24.6, 11.1, 4.8 Hz, 2H), 2.18 (qd, J=12.4, 4.4 Hz, 1H), 2.03-1.93 (m, 1H), 1.89 (dd, J=13.2, 3.6 Hz, 3H), 1.57-1.39 (m, 2H).

### Example 81: Harness 4: 1-(4-(2,6-dioxopiperidin-3-yl)phenyl)piperidine-4-carbaldehyde (Har04)

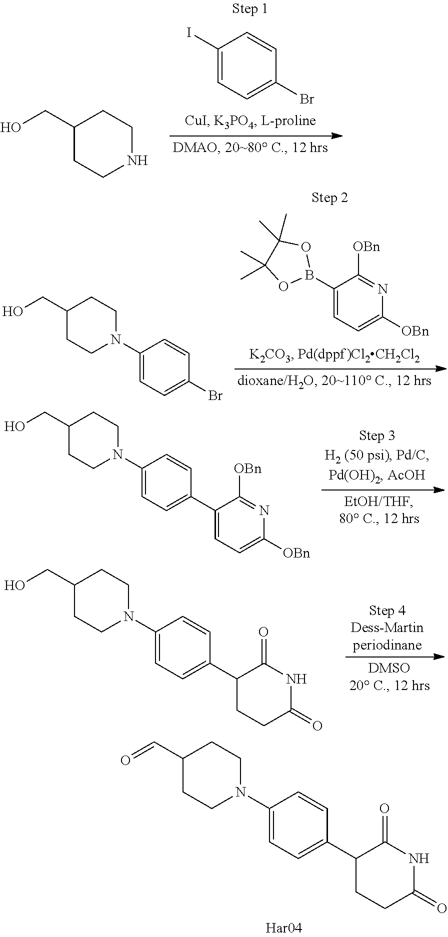

Step 1: To a solution of compound 1-bromo-4-iodobenzene (50.0 g, 176 mmol, 1.00 eq) in DMSO (250 mL) was added piperidin-4-ylmethanol (26.4 g, 229 mmol, 1.30 eq), K₃PO₄ (75.0 g, 353 mmol, 2.00 eq), CuI (6.73 g, 35.3 mmol, 0.200 eq) and L-proline (4.64 g, 35.3 mmol, 0.200 eq) at 20° C. under N₂. The reaction mixture was stirred at 80° C. for 12 hrs under N₂. The mixture was cooled to 20° C. and poured into water (1.00 L). The mixture was stirred at 0.5 hr. The mixture was extracted with ethyl acetate (500 mL*3). The organic layers were washed with NH₃.H₂O in H₂O (100 ml NH₃.H₂O in 700 mL H₂O) (250 mL*3). The organic layers were washed with brine (1000 mL), dried over Na₂SO₄, filtered and concentrated. The residue was purified by column chromatography (SiO₂, Petroleum ether/Ethyl acetate=20/1 to 2/1, R_{f}=0.30). Desired product (31.0 g, 87.0 mmol, 49.2% yield) was obtained as yellow solid. LCMS: m/z=270.0 (M+H)⁺.

Step 2: To a solution of (1-(4-bromophenyl)piperidin-4-yl)methanol (30.0 g, 84.2 mmol, 1.00 eq) and 2,6-bis(benzyloxy)-3-(4,4,5-trimethyl-1,3,2-dioxaborolan-2-yl)pyridine (35.1 g, 84.2 mmol, 1.00 eq) in dioxane (300 mL) and H₂O (60.0 mL) was added K₂CO₃ (34.9 g, 252 mmol, 3.00 eq) and Pd(dppf)Cl₂.CH₂Cl₂ (6.88 g, 8.43 mmol, 0.100 eq) at 20° C. under N₂. The reaction was stirred at 110° C. for 12 hrs under N₂. The reaction was cooled to 20° C. and filtered. The filtrate was concentrated. The crude product was purified by re-crystallization from MeOH (100 mL) at 20° C. for 30 mins. The mixture was filtered and the filter cake was purified by column chromatography (SiO₂, Petroleum ether/Ethyl acetate=10/1 to 1/1, R_{f}=0.30). Desired product (22.5 g, 44.8 mmol, 53.2% yield) was obtained as yellow solid. LCMS: m/z=481.2 (M+H)⁺.

Step 3: To a solution of (1-(4-(2,6-bis(benzyloxy)pyridin-3-yl)phenyl)piperidin-4-yl)methanol (20.0 g, 39.8 mmol, 1.00 eq) and AcOH (7.18 g, 119 mmol, 6.84 mL, 3.00 eq) in THF (200 mL) and EtOH (200 mL) was added Pd/C (5.00 g, 39.8 mmol, 10% purity, 1.00 eq) and Pd(OH)₂ (4.79 g, 34.1 mmol, 8.56e-1 eq) under N₂. The suspension was degassed under vacuum and purged with H₂ several times. The mixture was stirred under H₂ (50 psi) at 80° C. for 12 hrs. The mixture was cooled to 20° C. and filtered. The filtrate was concentrated. The crude product was triturated with Petroleum ether/Ethyl acetate=5/1 (200 mL) at 20° C. for 30 mins. Desired product (8.00 g, 24.2 mmol, 60.7% yield) was obtained as white solid. LCMS: m/z=303.2 (M+H)⁺.

Step 4: To a solution of 3-(4-(4-(hydroxymethyl)piperidin-1-yl)phenyl)piperidine-2,6-dione (8.60 g, 28.4 mmol, 1.00 eq) in DMSO (90.0 mL) was slowly added DESS-MARTIN PERIODINANE (24.1 g, 56.8 mmol, 17.6 mL, 2.00 eq) at 20° C. The reaction was stirred at 20° C. for 12 hrs. The mixture was adjusted with saturated aqueous Na₂CO₃ until pH=10 and the aqueous layer was extracted with ethyl acetate (350 mL*3). The combined organic layers were washed with Na₂S₂O₃ solution (500 mL) and brine (500 mL), dried over Na₂SO₄, concentrated under vacuum. The crude product was triturated with ethyl acetate (100 mL) at 20° C. for 30 mins. The title compound (7.18 g, 22.8 mmol, 48.1% yield) was obtained as off-white solid. LCMS: m/z=299.1 (M−H)⁺. ¹H NMR: (400 MHz, DMSO-d₆) δ 11.00-10.57 (m, 1H), 9.80-9.35 (m, 1H), 7.09-6.98 (m, 2H), 6.95-6.83 (m, 2H), 3.79-3.66 (m, 1H), 3.63-3.49 (m, 2H), 2.89-2.72 (m, 2H), 2.70-2.59 (m, 1H), 2.48-2.38 (m, 2H), 2.17-1.89 (m, 4H), 1.65-1.47 (m, 2H).

### (S)-1-(4-(2,6-dioxopiperidin-3-yl)phenyl)piperidine-4-carbaldehyde

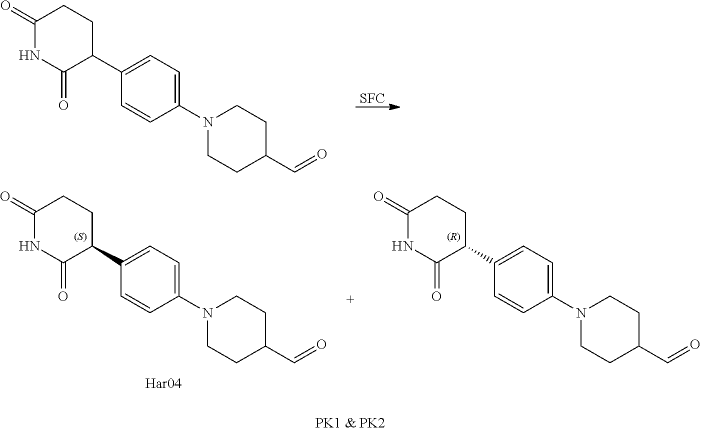

The crude product was purified by prep-SFC (column: DAICEL CHIRALPAK AD (250 mm*30 mm, 10 um); mobile phase: [Neu-IPA]; B %: 65%-65%, 4.5; 650 mins) to get the PK1 (S)-1-(4-(2,6-dioxopiperidin-3-yl)phenyl)piperidine-4-carbaldehyde (Har04) (13.64 g, 45.41 mmol, 48.7% yield) as yellow solid and crude peak 2. The crude peak 2 was purified by prep-SFC (column: DAICEL CHIRALPAK AD (250 mm*30 mm, 10 um); mobile phase: [IPA-ACN]; B %: 65%-65%, 4; 580 mins) to get the PK2 (8.13 g, 27.07 mmol, 29.04% yield) as off-white solid. PK1 LCMS: C₁₇H₂₀N₂O₃ requires: 300.2; found: m/z=299.1 [M−H]⁻. PK2 LCMS: C17H20N2O3 requires: 300.2; found: m/z=299.1 [M−H]⁻.

### Example 82: Harness 5: 1-(3-(2,6-dioxopiperidin-3-yl)phenyl)piperidine-4-carbaldehyde

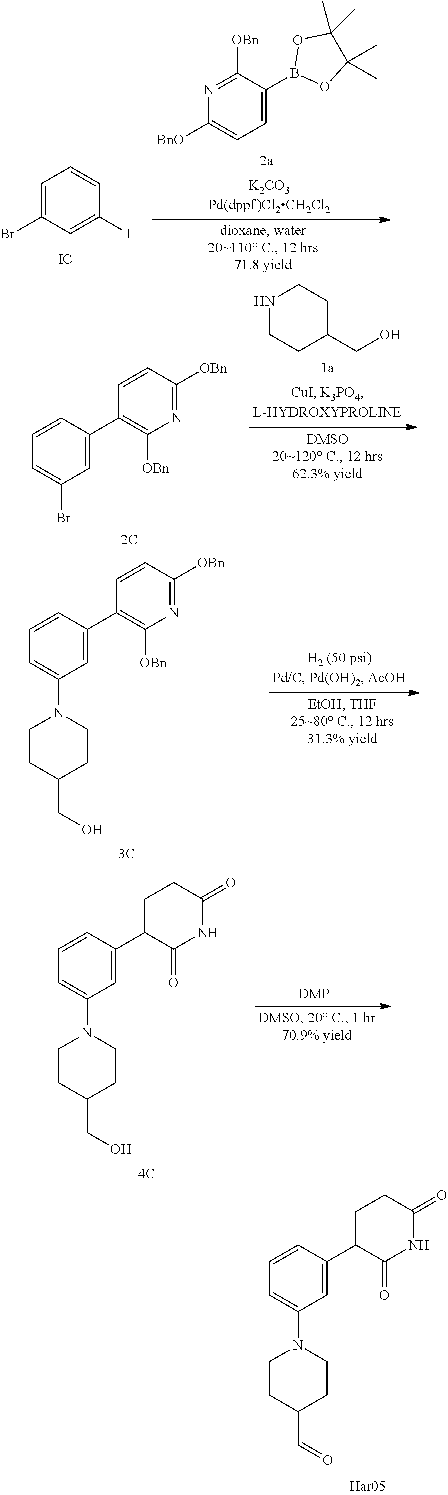

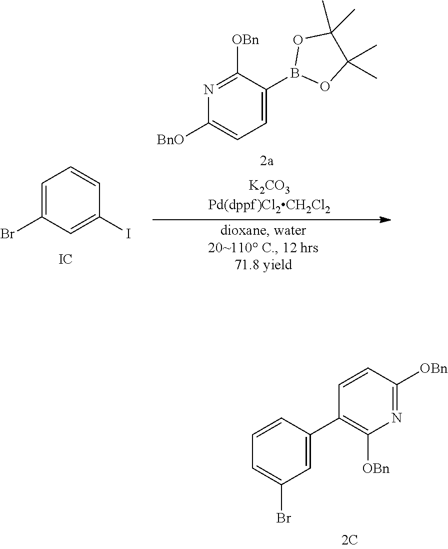

To a solution of Cpd_1C (40.0 g, 141 mmol, 18.0 mL, 1.00 eq) and Cpd_2a (59.0 g, 141 mmol, 1.00 eq) in dioxane (400 mL) and H₂O (80.0 mL) was added K₂CO₃ (58.6 g, 424 mmol, 3.00 eq) and Pd(dppf)Cl₂CH₂Cl₂ (11.5 g, 14.1 mmol, 0.10 eq) at 20° C. under N₂. The reaction was stirred at 110° C. for 12 hrs under N₂. LCMS showed that Cpd_1C was consumed and 71% desired peak (RT=1.079 min) was detected. The reaction was cooled to 20° C. The residue was poured into water (800 mL) and extracted with ethyl acetate (800 mL*4). The combined organic layers were washed with brine (100 mL), dried over Na₂SO₄, filtered and concentrated. The crude product was purified by MPLC (SiO₂, Petroleum ether/Ethyl acetate=1/0˜0/1) (Petroleum ether/Ethyl acetate=10/1, R_{f}=0.50). Cpd_2C (51.0 g, 114 mmol, 71.8% yield) was obtained as light yellow oil. LCMS: product: RT=1.079 mins, m/z=448.0 (M+H)⁺

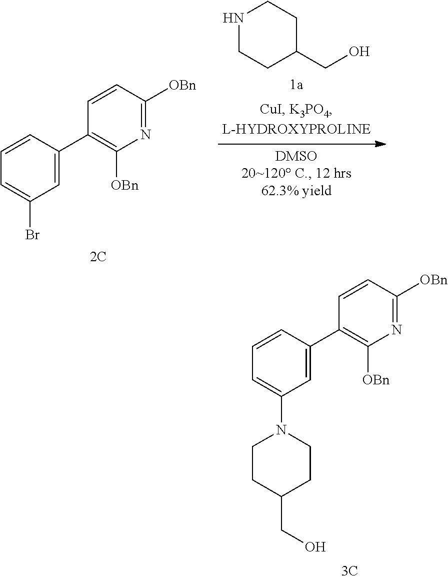 [SMILES: OCC1CCNCC1 ; Brc1cccc(-c2ccc(OCc3ccccc3)nc2OCc2ccccc2)c1 ; OCC1CCN(c2cccc(-c3ccc(OCc4ccccc4)nc3OCc3ccccc3)c2)CC1]

To a solution of Cpd_1a (21.1 g, 183 mmol, 2.00 eq) and Cpd_2C (41.0 g, 91.8 mmol, 1.0 eq) in DMSO (410 mL) was added CuI (12.2 g, 64.3 mmol, 0.70 eq), L-hydroxyproline (6.02 g, 45.9 mmol, 0.50 eq) and K₃PO₄ (39.0 g, 183 mmol, 2.00 eq) at 20° C. under N₂. The mixture was stirred at 120° C. for 12 hrs under N₂. LCMS showed that Cpd_2C was consumed and 100% desired mass (RT=0.934 min) was detected. The mixture was poured into H₂O (1000 mL). The mixture was added ethyl acetate (500 mL). The mixture was filtered to get the filtrate. The mixture was extracted with ethyl acetate (500 mL*2). The combined organic layer was washed with brine (200 mL), dried over Na₂SO₄ and concentrated. The crude product was purified by MPLC (SiO₂, Petroleum ether/Ethyl acetate=1/0 to 0/1) (Petroleum ether/Ethyl acetate=1/1, R_{f}=0.50). Cpd_3C (35.0 g, 71.0 mmol, 62.3% yield, 97.5% purity) was obtained as light yellow oil. LCMS: product: RT=0.803 min, m/z=481.2 (M+H)⁺; ¹H NMR: (400 MHz, DMSO d₆) δ 7.73 (d, J=8.2 Hz, 1H), 7.26-7.55 (m, 10H), 7.19 (t, J=8.0 Hz, 1H), 7.01-7.12 (m, 1H), 6.77-6.95 (m, 2H), 6.54 (d, J=8.0 Hz, 1H), 5.38 (d, J=10.6 Hz, 4H), 4.48 (t, J=5.2 Hz, 1H), 3.59 (br d, J=12.2 Hz, 2H), 3.27 (t, J=5.8 Hz, 2H), 2.55 (br d, J=2.0 Hz, 2H), 1.70 (br d, J=10.8 Hz, 2H), 1.40-1.56 (m, 1H), 1.09-1.32 (m, 2H)

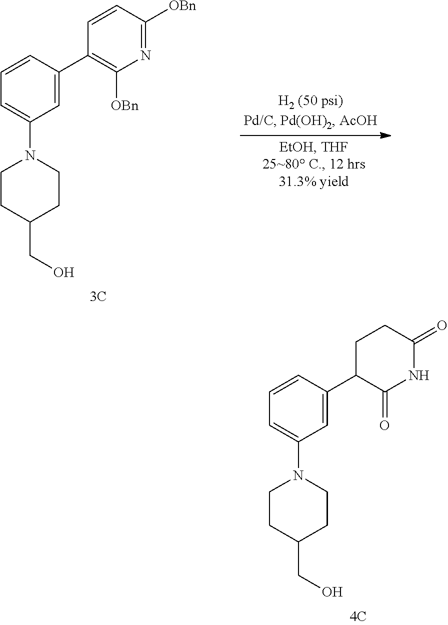 [SMILES: OCC1CCN(c2cccc(-c3ccc(OCc4ccccc4)nc3OCc3ccccc3)c2)CC1 ; O=C1CCC(c2cccc(N3CCC(CO)CC3)c2)C(=O)N1]

To a solution of Cpd_3C (25.0 g, 50.7 mmol, 97.5% purity, 1.00 eq) and AcOH (9.14 g, 152 mmol, 8.70 mL, 3.00 eq) in THF (250 mL) and EtOH (250 mL) was added Pd/C (6.25 g, 10.0% purity) and Pd(OH)₂ (6.25 g, 20.0% purity) under N₂ at 25° C. The suspension was degassed under vacuum and purged with H₂ several times. The mixture was stirred under H₂ (50 psi) at 80° C. for 12 hrs. LCMS showed that Cpd_3C was consumed and 82.3% desired mass (RT=0.740 min) was detected. The mixture was cooled to 20° C. and filtered. The filtrate was concentrated. The crude product was purified by MPLC (SiO₂, Petroleum ether/Ethyl acetate=1/0 to Dichloromethane/Methanol=10/1) (Dichloromethane/Methanol=10/1, R_{f}=0.50). Cpd_4C (4.80 g, 15.8 mmol, 31.30% yield, 100% purity) was obtained as light yellow solid. LCMS: product: RT=0.731 min, m/z=303.1 (M+H)⁺; ¹H NMR: (400 MHz, CDCl₃) δ 8.14 (br s, 1H), 7.20-7.27 (m, 1H), 6.89 (br d, J=8.0 Hz, 1H), 6.78 (s, 1H), 6.66 (d, J=7.2 Hz, 1H), 3.66-3.83 (m, 3H), 3.56 (d, J=6.4 Hz, 2H), 2.54-2.86 (m, 4H), 2.16-2.38 (m, 2H), 1.86 (br d, J=12.2 Hz, 2H), 1.58-1.78 (m, 1H), 1.32-1.50 (m, 2H).

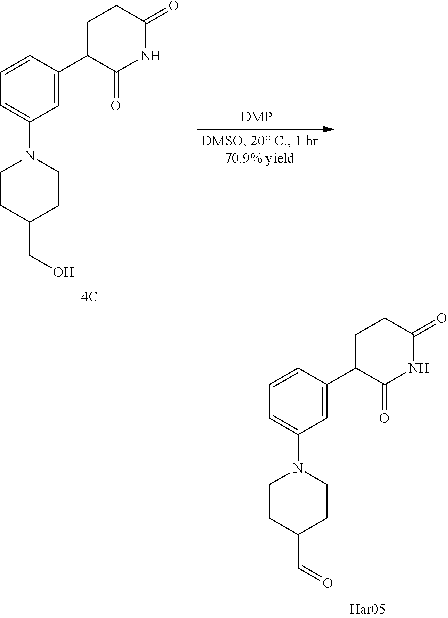 [SMILES: O=C1CCC(c2cccc(N3CCC(CO)CC3)c2)C(=O)N1 ; O=CC1CCN(c2cccc(C3CCC(=O)NC3=O)c2)CC1]

To a solution of Cpd_4C (4.80 g, 15.8 mmol, 1.00 eq) in DMSO (48.0 mL) was slowly added DMP (1.35 g, 3.17 mmol, 983 μL, 0.20 eq) at 20° C. The reaction was stirred at 20° C. for 1 hr. TLC (Petroleum ether/Ethyl acetate=0/1) showed Cpd_4C (R_{f}=0.40) was consumed and a new spot (R_{f}=0.50) was detected. The mixture was poured into saturated Na₂CO₃ aqueous solution (200 mL). The mixture was adjusted to pH˜10 with Na₂CO₃ aqueous solution. The mixture was extracted with DCM (200 mL*3). The combined organic layer was washed with Na₂S₂O₃ solution (100 mL) and brine (100 mL), dried over Na₂SO₄, concentrated under vacuum. The crude product was triturated with Ethyl acetate and hexane (Ethyl acetate/hexane=1/1, 25 V) at 25° C. for 30 mins. Har05 (4.84 g, 15.8 mmol, 70.9% yield, 98.5% purity) was obtained as brown solid. LCMS: EW34610-17-P1C₁, product: RT=0.748 min, m/z=301.1 (M+H)⁺; ¹H NMR: (400 MHz, CDCl₃) δ 9.72 (s, 1H), 8.08 (br s, 1H), 7.27 (s, 1H), 6.89 (dd, J=8.4, 1.6 Hz, 1H), 6.77 (s, 1H), 6.69 (d, J=7.2 Hz, 1H), 3.75 (dd, J=9.2, 5.2 Hz, 1H), 3.62 (dt, J=12.2, 4.0 Hz, 2H), 2.83-2.95 (m, 2H), 2.59-2.80 (m, 2H), 2.37-2.51 (m, 1H), 2.18-2.34 (m, 2H), 1.95-2.12 (m, 2H), 1.72-1.86 (m, 2H).

### Hook Syntheses

### Example 83: Hooks for CDK 4/6

### 6-acetyl-8-cyclopentyl-5-methyl-2-((5-(piperazin-1-yl)pyridin-2-yl)amino)pyrido[2,3-d]pyrimidin-7(8H)-one (H01)

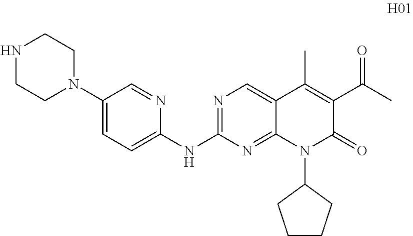 [SMILES: CC(=O)c1c(C)c2cnc(Nc3ccc(N4CCNCC4)cn3)nc2n(C2CCCC2)c1=O]

6-acetyl-8-cyclopentyl-5-methyl-2-((5-(piperazin-1-yl)pyridin-2-yl)amino)pyrido[2,3-d]pyrimidin-7(8H)-one (H01) was purchased from Combi-blocks (catalog number: QJ-2085, batch B52069)

### 7-cyclopentyl-N,N-dimethyl-2-((5-(piperazin-1-yl)pyridin-2-yl)amino)-7H-pyrrolo[2,3-d]pyrimidine-6-carboxamide (H02)

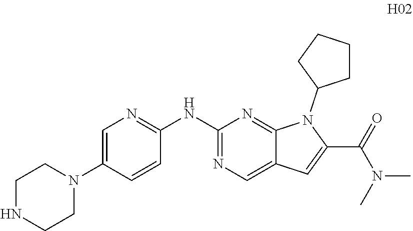 [SMILES: CN(C)C(=O)c1cc2cnc(Nc3ccc(N4CCNCC4)cn3)nc2n1C1CCCC1]

7-cyclopentyl-N,N-dimethyl-2-((5-(piperazin-1-yl)pyridin-2-yl)amino)-7H-pyrrolo[2,3-d]pyrimidine-6-carboxamide (H02) was purchased from Combi-blocks (catalog number: HD-1354, batch B52670)

### Example 84: Hooks for SHP2

tert-butyl (1-(6-amino-5-((3-amino-2-chlorophenyl)thio)pyrazin-2-yl)-4-methylpiperidin-4-yl)carbamate (H03)

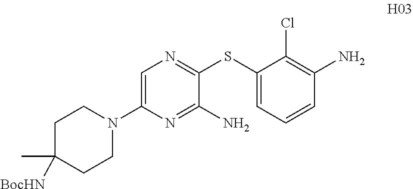 [SMILES: CC1(NC(=O)OC(C)(C)C)CCN(c2cnc(Sc3cccc(N)c3Cl)c(N)n2)CC1]

Compound H03 was synthesized according to: J. Med. Chem. 2020, 63, 7510-7528, incorporated herein by reference in its entirety.

### 6-(4-amino-4-methylpiperidin-1-yl)-3-(3,4-dichlorophenyl)pyrazin-2-amine (H04)

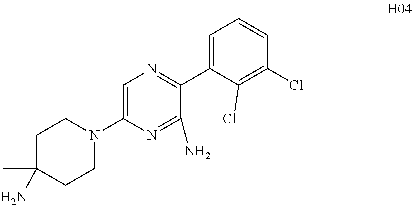 [SMILES: CC1(N)CCN(c2cnc(-c3cccc(Cl)c3Cl)c(N)n2)CC1]

Compound H04 was purchased from 2HBiochem.

### Example 85: Hooks for FGFR1/3 Fusions (H05)

### Des-ethyl Infigratinib

### 3-(2,6-dichloro-3,5-dimethoxyphenyl)-1-methyl-1-(6-((4-(piperazin-1-yl)phenyl)amino)pyrimidin-4-yl)urea

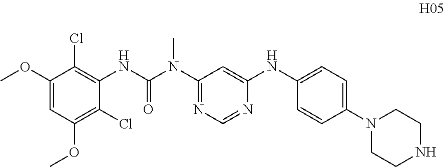 [SMILES: COc1cc(OC)c(Cl)c(NC(=O)N(C)c2cc(Nc3ccc(N4CCNCC4)cc3)ncn2)c1Cl]

### Step 1: tert-butyl 4-(4-((6-(methylamino)pyrimidin-4-yl)amino)phenyl)piperazine-1-carboxylate

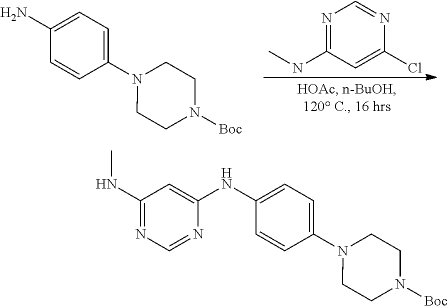

To a solution of 6-chloro-N-methylpyrimidin-4-amine (2.59 g, 18.0 mmol, 1.00 eq) in n-BuOH (100 mL) was add tert-butyl 4-(4-aminophenyl)piperazine-1-carboxylate (5.00 g, 18.0 mmol, 1.00 eq) and AcOH (54.1 mg, 901 μmol, 51.6 μL, 0.05 eq). Then the reaction mixture was stirred at 120° C. for 16 hrs under N₂. The reaction mixture was concentrated directly to give a residue. To the residue was added cool water (500 mL) under stirring, and the pH of the mixture was adjusted to 8˜9 by adding Sat. NaHCO₃.aq under stirring. Then the mixture was extracted with ethyl acetate (220 mL*5). The combined organic layers were washed with brine (300 mL), dried over Na₂SO₄ (about 50.0 g), filtered and concentrated. The crude product was purified by silica gel chromatography (Dichloromethane:Methanol=95.0%:5.00%) to afford tert-butyl 4-(4-((6-(methylamino)pyrimidin-4-yl)amino)phenyl)piperazine-1-carboxylate (4.50 g, 11.1 mmol, 61.3% yield). LCSM C₂₀H₂₈N₆O₂ requires: 384, found: m/z=385 [M+H]⁺.

### Step 2: tert-butyl 4-(4-((6-(3-(2,6-dichloro-3,5-dimethoxyphenyl)-1-methylureido)pyrimidin-4-yl)amino)phenyl)piperazine-1-carboxylate

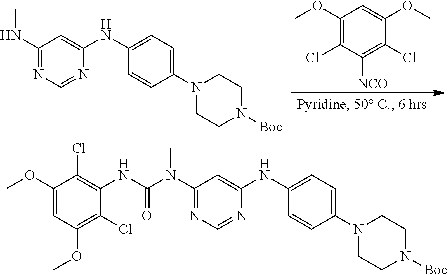

A mixture of tert-butyl 4-(4-((6-(methylamino)pyrimidin-4-yl)amino)phenyl)piperazine-1-carboxylate (4.00 g, 10.4 mmol, 1.00 eq) in dry Pyridine (20.0 mL) under N₂ was warmed to 50° C. To the mixture was added a solution of 2,4-dichloro-3-isocyanato-1,5-dimethoxybenzene (3.87 g, 15.6 mmol, 1.50 eq) under N₂. The reaction mixture was stirred at 50° C. under N₂ for 6 hrs. The reaction mixture was concentrated in vacuo to give residue. The residue was purified by silica gel chromatography eluted with Dichloromethane:Methanol=95:5 to give tert-butyl 4-(4-((6-(3-(2,6-dichloro-3,5-dimethoxyphenyl)-1-methylureido)pyrimidin-4-yl)amino)phenyl)piperazine-1-carboxylate (2.50 g, 3.95 mmol, 38.0% yield). LCSM C₂₉H₃₅C₁₂N₇O₅ requires: 631, found: m/z=632 [M+H]⁺.

### Step 3: 3-(2,6-dichloro-3,5-dimethoxyphenyl)-1-methyl-1-(6-((4-(piperazin-1-yl)phenyl)amino)pyrimidin-4-yl)urea

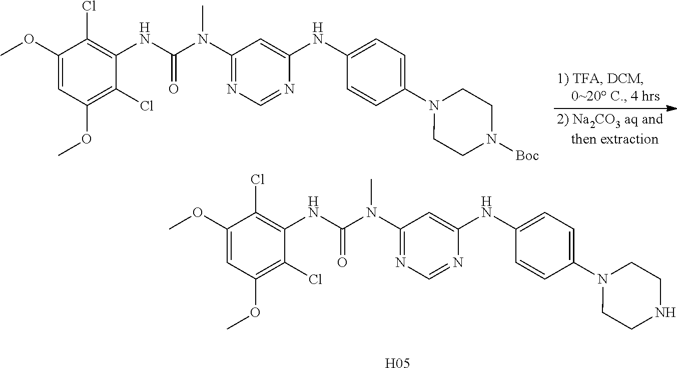 [SMILES: COc1cc(OC)c(Cl)c(NC(=O)N(C)c2cc(Nc3ccc(N4CCN(C(=O)OC(C)(C)C)CC4)cc3)ncn2)c1Cl ; COc1cc(OC)c(Cl)c(NC(=O)N(C)c2cc(Nc3ccc(N4CCNCC4)cc3)ncn2)c1Cl ; "H05"]

To a solution of tert-butyl 4-(4-((6-(3-(2,6-dichloro-3,5-dimethoxyphenyl)-1-methylureido)pyrimidin-4-yl)amino)phenyl)piperazine-1-carboxylate (2.50 g, 3.95 mmol, 1.00 eq) in DCM (33.0 mL) was added TFA (16.9 g, 149 mmol, 11.0 mL, 37.6 eq) at 0° C. under N₂. The reaction mixture was stirred at 20° C. under N₂ for 4 hrs. The reaction was concentrated to give a residue. The pH of the residue was adjusted to pH=10˜11 by adding 10.0% Sat.Na₂CO₃ aq at 0° C. under stirring. To the mixture was added ethyl acetate (10.0 mL) under stirring, and ultrasound for 15 mins at 25° C., then the mixture was filtered, and the filter cake was washed with water (10.0 mL*2). The filter cake was dissolved in dichloromethane: Methanol=3/1 (1.00 L) under stirring. The resulting mixture was filtered. The filtrate was washed with H₂O (100 mL). The filtrate was concentrated to give the crude product. The crude product was triturated with methanol (10.0 mL) under stirring, filtered, and the filter cake was washed with n-hexane (30.0 mL*2), and the filter cake was dried under reduced pressure to give 3-(2,6-dichloro-3,5-dimethoxyphenyl)-1-methyl-1-(6-((4-(piperazin-1-yl)phenyl)amino)pyrimidin-4-yl)urea (H05) (1.10 g, 2.02 mmol, 51.1% yield). ¹H NMR: (400 MHz DMSO-d₆) δ 9.43 (s, 1H), 8.38 (s, 1H), 7.42-7.40 (m, 2H), 6.93 (s, 1H), 6.92-6.90 (m, 2H), 6.41 (s, 1H), 3.94 (s, 6H), 3.30 (s, 3H), 3.05-3.03 (m, 4H), 2.90-2.88 (m, 4H). LCSM C₂₄H₂₇C₁₂N₇O₃ requires: 531, found: m/z=532 [M+H]⁺.

### Example 86: Hook for MDM2 (H06)

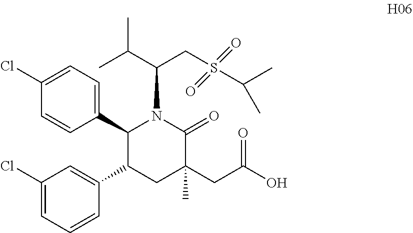

Compound H06 (avtemadlin; AMG-232) was purchased from MedChemExpress.

### Example 87: Hook for Mpro (H08)

### Synthesis of (4R)-2-oxo-3,4-dihydro-1H-quinoline-4-carboxylic acid & (45)-2-oxo-3,4-dihydro-1H-quinoline-4-carboxylic acid

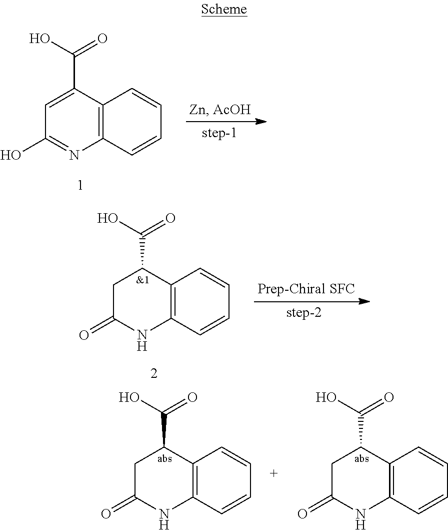 [SMILES: O=C(O)c1cc(O)nc2ccccc12 ; O=C1C[C@H](C(=O)O)c2ccccc2N1 ; O=C1C[C@@H](C(=O)O)c2ccccc2N1 ; O=C1C[C@H](C(=O)O)c2ccccc2N1]

### Step 1: Synthesis of rac-(R)-2-oxo-1,2,3,4-tetrahydroquinoline-4-carboxylic acid (2)

To a stirred solution of 2-hydroxyquinoline-4-carboxylic acid (50 g, 264.31 mmol, 1 equiv) in AcOH (400 mL) was added Zn (34.7 g, 528.625 mmol, 2 equiv) in portions at room temperature. The resulting mixture was stirred at 60° C. overnight under nitrogen atmosphere. The resulting mixture was filtered. The filtrate was collected and concentrated under vacuum. The precipitated solids were collected by filtration and washed with water (3×100 mL). This resulted in rac-(R)-2-oxo-1,2,3,4-tetrahydroquinoline-4-carboxylic acid (40 g, 79.15%) as a yellow solid. MS (ESI) calc'd for (C₁₀H₉NO₃) [M+1]⁺, 192.1; found, 192.1

### Step 2: Synthesis of (4R)-2-oxo-3,4-dihydro-1H-quinoline-4-carboxylic acid & (4S)-2-oxo-3,4-dihydro-1H-quinoline-4-carboxylic acid

The racemic product (40 g) was separated by Prep-Chiral-SFC with the following conditions: Column: CHIRALPAK IG, 5*25 cm, 5 um; Mobile Phase A: CO₂, Mobile Phase B: MeOH (0.1% 2M NH₃-MeOH); Flow rate: 200 mL/min; Gradient: isocratic 50% B; Column Temperature (° C.): 35; Back Pressure (bar): 100; Wave Length: 220 nm; RT1 (min): 2.95; RT2 (min): 4.13; Sample Solvent: MeOH (0.1% 2M NH₃-MeOH); Injection Volume: 19.99 mL; Number Of Runs: 60. This resulted in 12.5 g (62.5%) of (4R)-2-oxo-3,4-dihydro-1H-quinoline-4-carboxylic acid (PH-NUR-P01-021) as a yellow solid. MS (ESI) calc'd for (C₁₀H₉NO₃) [M+1]⁺, 192.1; found, 191.9. NMR (400 MHz, Methanol-d₄) δ 7.39-7.33 (m, 1H), 7.21-7.13 (m, 1H), 7.04-6.95 (m, 1H), 6.90-6.83 (m, 1H), 3.82-3.75 (m, 1H), 2.93-2.83 (m, 1H), 2.75-2.64 (m, 1H), and 13.7 g (68.5%) of (4S)-2-oxo-3,4-dihydro-1H-quinoline-4-carboxylic acid as a yellow solid. MS (ESI) calc'd for (C₁₀H₉NO₃) [M+1]⁺, 192.1; found, 191.9. NMR (400 MHz, Methanol-d₄) δ 7.39-7.32 (m, 1H), 7.24-7.13 (m, 1H), 7.04-6.95 (m, 1H), 6.90-6.83 (m, 1H), 3.83-3.75 (m, 1H), 2.93-2.83 (m, 1H), 2.75-2.64 (m, 1H).

### Synthesis of 4-[2-(7-chloro-1,3-benzothiazol-2-yl)phenoxy]-1-[(4S)-2-oxo-3,4-dihydro-1H-quinoline-4-carbonyl]piperidine-4-carboxylic acid (H08)

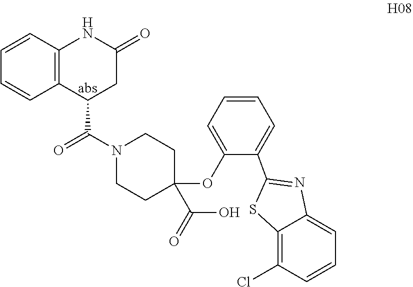 [SMILES: O=C1C[C@H](C(=O)N2CCC(Oc3ccccc3-c3nc4cccc(Cl)c4s3)(C(=O)O)CC2)c2ccccc2N1]

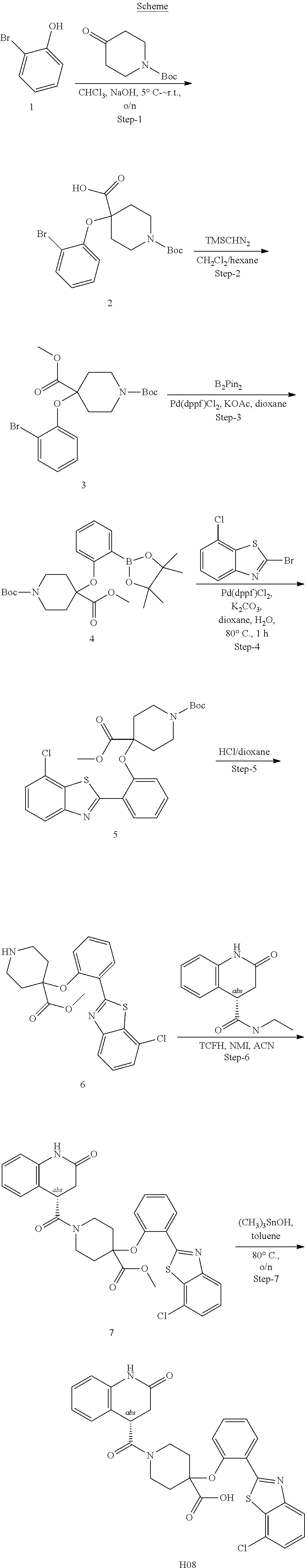

### Step 1: Synthesis of 4-(2-bromophenoxy)-1-(tert-butoxycarbonyl)piperidine-4-carboxylic acid (2)

To a mixture of 2-bromophenol (100 g, 0.578 mol, 1 equiv) and tert-butyl 4-oxopiperidine-1-carboxylate (345 g, 1.734 mol, 3 equiv) in THF (800 mL) were added NaOH (116 g, 2.89 mol, 5 equiv) at 0° C. The resulting mixture was stirred at 0° C. for 0.5 h. To the above mixture was added CHCl₃ (344 g, 2.89 mol, 5 equiv). The resulting mixture was stirred at room temperature overnight. The precipitated solids were collected by filtration and washed with DCM (3×200 mL). Then the solid was dissolved in water and washed with MTBE (3×200 mL). The combined water layers were acidified with HCl (2 N) to pH 3-4 and extracted with ethyl acetate. The combined organic layers were washed with brine, dried over anhydrous sodium sulfate, filtered, and concentrated under vacuum. This resulted in 4-(2-bromophenoxy)-1-(tert-butoxycarbonyl)piperidine-4-carboxylic acid (170 g, crude) as a light yellow oil. MS (ESI) calc'd for (C₁₇H₂₂BrNO₅) [M+1]⁺, 399.1; found, 400.2.

### Step 2: Synthesis of 1-tert-butyl 4-methyl 4-(2-bromophenoxy)piperidine-1,4-dicarboxylate (3)

To a mixture of 4-(2-bromophenoxy)-1-(tert-butoxycarbonyl)piperidine-4-carboxylic acid (15 g, 37.475 mmol) in DCM (200 mL) and MeOH (20 mL) were added TMSCHN₂ (93.69 mL, 187.375 mmol, 2M in Hexane) at 0° C. The resulting mixture was stirred at room temperature for 2 h. The resulting mixture was concentrated under reduced pressure. The residue was purified by silica gel column chromatography of [PE and EA] to provide 9 g (52%) of the title compound as a white solid. LCMS: (C₁₈H₂₄BrNO₅) desired mass=413.0; observed mass=414.0 [M+H]⁺.

### Step 3: Synthesis of 1-tert-butyl 4-methyl 4-[2-(4,4,5,5-tetramethyl-1,3,2-dioxaborolan-2-yl)phenoxy]piperidine-1,4-dicarboxylate (4)

To a mixture of 1-tert-butyl 4-methyl 4-(2-bromophenoxy)piperidine-1,4-dicarboxylate (5000 mg, 12.069 mmol, 1 equiv) and 4,4,5,5-tetramethyl-2-(tetramethyl-1,3,2-dioxaborolan-2-yl)-1,3,2-dioxaborolane (15323 mg, 60.345 mmol, 5 equiv) in 1,4-dioxane (50 mL) were added KOAc (2368 mg, 24.138 mmol, 2 equiv) and Pd(dppf)Cl₂.CH₂Cl₂ (1966 mg, 2.414 mmol, 0.2 equiv). The resulting mixture was stirred at 80° C. overnight under nitrogen atmosphere. The resulting mixture was concentrated under reduced pressure. The residue was purified by silica gel column chromatography, eluted with PE/EA (6/1) to afford 1-tert-butyl 4-methyl 4-[2-(4,4,5,5-tetramethyl-1,3,2-dioxaborolan-2-yl)phenoxy]piperidine-1,4-dicarboxylate (2.2 g, 31.61%) as a yellow oil MS (ESI) calc'd for (C₂₄H₃₆BNO₇) [M+1]⁺, 462.3; found, 462.2.

Step 4: Synthesis of 1-tert-butyl 4-methyl 4-[2-(7-chloro-1,3-benzothiazol-2-yl)phenoxy]piperidine-1,4-dicarboxylate (5):

To a mixture of 1-tert-butyl 4-methyl 4-[2-(4,4,5,5-tetramethyl-1,3,2-dioxaborolan-2-yl)phenoxy]piperidine-1,4-dicarboxylate (2.2 g, 4.769 mmol, 1 equiv) in 1,4-dioxane (30 mL) was added 2-bromo-7-chloro-1,3-benzothiazole (0.95 g, 3.815 mmol, 0.8 equiv), K₂CO₃ (1.98 g, 14.307 mmol, 3 equiv) in H₂O (2 mL) and Pd(dppf)Cl₂CH₂Cl₂ (0.78 g, 0.954 mmol, 0.2 equiv). The resulting mixture was stirred at 80° C. for 3h under nitrogen atmosphere. The resulting mixture was concentrated under reduced pressure. The residue was purified by silica gel column chromatography, eluted with PE/EA (5/1) to afford 1-tert-butyl 4-methyl 4-[2-(7-chloro-1,3-benzothiazol-2-yl)phenoxy]piperidine-1,4-dicarboxylate (930 mg, 38.77%) as a white solid. MS (ESI) calc'd for (C₂₅H₂₇ClN₂O₅S) [M+1]⁺, 503.1; found, 503.1.

### Step 5: Synthesis of methyl 4-[2-(7-chloro-1,3-benzothiazol-2-yl)phenoxy]piperidine-4-carboxylate (6)

A mixture of 1-tert-butyl 4-methyl 4-[2-(7-chloro-1,3-benzothiazol-2-yl)phenoxy]piperidine-1,4-dicarboxylate (930 mg, 1.849 mmol, 1 equiv) in HCl (gas) in 1,4-dioxane (20 mL, 4N) was stirred at room temperature for 1 h. The resulting mixture was concentrated under reduced pressure. This resulted in methyl 4-[2-(7-chloro-1,3-benzothiazol-2-yl)phenoxy]piperidine-4-carboxylate (850 mg, crude) as a white solid. The crude product was used in the next step directly without further purification. MS (ESI) calc'd for (C₂₀H₁₉ClN₂O₃S) [M+1]⁺, 403.1; found, 403.1.

### Step 6: Synthesis of methyl 4-[2-(7-chloro-1,3-benzothiazol-2-yl)phenoxy]-1-[(4S)-2-oxo-3,4-dihydro-1H-quinoline-4-carbonyl]piperidine-4-carboxylate (7)

To a mixture of methyl 4-[2-(7-chloro-1,3-benzothiazol-2-yl)phenoxy]piperidine-4-carboxylate (2300 mg, 5.709 mmol, 1 equiv) and (4S)-2-oxo-3,4-dihydro-1H-quinoline-4-carboxylic acid (1091 mg, 5.709 mmol, 1 equiv) in ACN (30 mL) were added NMI (1406 mg, 17.127 mmol, 3 equiv) and TCFH (1601 mg, 5.709 mmol, 1 equiv) dropwise at 0° C. The resulting mixture was stirred at 0° C. for 1 h under nitrogen atmosphere. The resulting mixture was concentrated under reduced pressure. The residue was purified by silica gel column chromatography, eluted with CH₂Cl₂/MeOH (10/1) to afford methyl 4-[2-(7-chloro-1,3-benzothiazol-2-yl)phenoxy]-1-[(4S)-2-oxo-3,4-dihydro-1H-quinoline-4-carbonyl]piperidine-4-carboxylate (1500 mg, 45.61%) as a white solid. MS (ESI) calc'd for (C₃₀H₂₆ClN₃O₅S) [M+1]⁺, 576.1; found, 576.1.

### Step 7: Synthesis of 4-[2-(7-chloro-1,3-benzothiazol-2-yl)phenoxy]-1-[(4S)-2-oxo-3,4-dihydro-1H-quinoline-4-carbonyl]piperidine-4-carboxylic acid (H08)

To a mixture of methyl 4-[2-(7-chloro-1,3-benzothiazol-2-yl)phenoxy]-1-[(4S)-2-oxo-3,4-dihydro-1H-quinoline-4-carbonyl]piperidine-4-carboxylate (1500 mg, 2.604 mmol, 1 equiv) in toluene (45 mL) was added (CH₃)3SnOH (4708 mg, 26.040 mmol, 10 equiv). The resulting mixture was stirred at 80° C. for 2 days. The resulting mixture was concentrated under reduced pressure. The residue was purified by reverse flash chromatography with CH₃CN/H₂O (1/1) to afford 4-[2-(7-chloro-1,3-benzothiazol-2-yl)phenoxy]-1-[(4S)-2-oxo-3,4-dihydro-1H-quinoline-4-carbonyl]piperidine-4-carboxylic acid (750 mg, 51.25%) as a white solid. MS (ESI) calc'd for (C₂₉H₂₄ClN₃O₅S) [M+1]⁺, 562.1; found, 562.2. ¹H NMR (400 MHz, Methanol-d₄) δ 8.44-8.36 (m, 1H), 8.02-7.96 (m, 1H), 7.59-7.51 (m, 1H), 7.51-7.42 (m, 2H), 7.30-7.00 (m, 4H), 6.98-6.76 (m, 2H), 4.59-4.53 (m, 1H), 4.27-4.06 (m, 2H), 4.01-3.78 (m, 1H), 3.64-3.46 (m, 1H), 2.88-2.63 (m, 2H), 2.60-2.30 (m, 4H).

### Example 88: Hooks for MEK

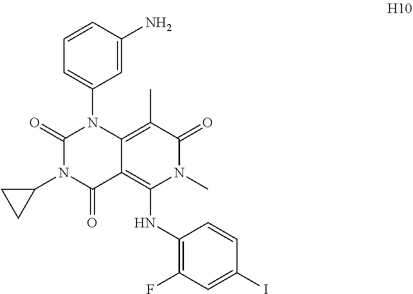 [SMILES: Cc1c(=O)n(C)c(Nc2ccc(I)cc2F)c2c(=O)n(C3CC3)c(=O)n(-c3cccc(N)c3)c12]

### Synthesis of H10: 1-(3-aminophenyl)-3-cyclopropyl-5-((2-fluoro-4-iodophenyl)amino)-6,8-dimethylpyrido[4,3-d]pyrimidine-2,4,7(1H,3H,6H)-trione (H10)

1-(3-aminophenyl)-3-cyclopropyl-5-((2-fluoro-4-iodophenyl)amino)-6,8-dimethylpyrido[4,3-d]pyrimidine-2,4,7(1H,3H,6H)-trione was synthesized in three steps from trametinib (purchased from Combi-Blocks, product number: HB-6465, batch number: B52129) according to WO2021142345A1.

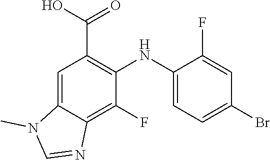 [SMILES: Cn1cnc2c(F)c(Nc3ccc(Br)cc3F)c(C(=O)O)cc21]

### Synthesis of 5-((4-bromo-2-fluorophenyl)amino)-4-fluoro-1-methyl-1H-benzo[d]imidazole-6-carboxylic acid

Binimetinib (3.92 g, 8.88 mmol) in aqueous 1 M hydrogen chloride (47.2 mL, 1.72 g, 47.19 mmol) was heated to 85° C. for 48 h. The reaction was cooled to room temperature and neutralized with aqueous 1 M NaOH to pH 6. The resulting precipitate was collected by filtration and lyophilized to provide 5-((4-bromo-2-fluorophenyl)amino)-4-fluoro-1-methyl-1H-benzo[d]imidazole-6-carboxylic acid (2.36 g, 69.51%) as a white solid. LCMS: C₁₅H₁₀BrF₂N₃O₂ requires: 381.0, found: m/z=382.2 [M+H]⁺.

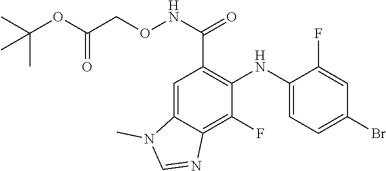 [SMILES: Cn1cnc2c(F)c(Nc3ccc(Br)cc3F)c(C(=O)NOCC(=O)OC(C)(C)C)cc21]

### Synthesis of tert-butyl 2-((5-((4-bromo-2-fluorophenyl)amino)-4-fluoro-1-methyl-1H-benzo[d]imidazole-6-carboxamido)oxy)acetate

5-((4-bromo-2-fluorophenyl)amino)-4-fluoro-1-methyl-1H-benzo[d]imidazole-6-carboxylic acid (1600.0 mg, 4.1867 mmol), tert-butyl 2-(aminooxy)acetate (1200.0 mg, 8.1536 mmol), ({[3-(dimethylamino)propyl]imino}methylidene)(ethyl)amine hydrochloride (882.3 mg, 4.603 mmol), and hydroxybenzotriazole hydrate (349.5 mg, 2.282 mmol) were dissolved in DMF (10.0 mL). Triethylamine (3.00 mL, 2.18 g, 21.5 mmol) was added and the reaction stirred at room temperature overnight. The reaction was concentrated and diluted with ethyl acetate and brine. The aqueous phase was extracted with ethyl acetate (3×) and the combined organic phases were dried over sodium sulfate, filtered, concentrated, and purified by flash chromatography on a 40 g silica column, eluted by gradient elution with 0 to 20% MeOH/EtOAc to provide tert-butyl 2-((5-((4-bromo-2-fluorophenyl)amino)-4-fluoro-1-methyl-1H-benzo[d]imidazole-6-carboxamido)oxy)acetate (326 mg, 15.23%) as a yellow solid. LCMS: C₂₁H₂₁BrF₂N₄O₄ requires: 512.1, found: m/z=513.3 [M+H]⁺.

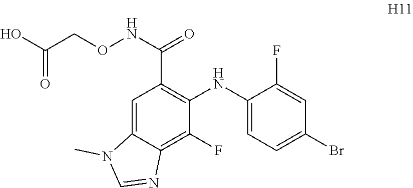 [SMILES: Cn1cnc2c(F)c(Nc3ccc(Br)cc3F)c(C(=O)NOCC(=O)O)cc21]

### Synthesis of H11: 2-((5-((4-bromo-2-fluorophenyl)amino)-4-fluoro-1-methyl-1H-benzo[d]imidazole-6-carboxamido)oxy)acetic acid

To a stirred solution of tert-butyl 2-((5-((4-bromo-2-fluorophenyl)amino)-4-fluoro-1-methyl-1H-benzo[d]imidazole-6-carboxamido)oxy)acetate (835.0 mg, 1.633 mmol) in methylene chloride (28.0 mL, 37.0 g, 435 mmol) was added trifluoroacetic acid (7.00 mL, 10.4 g, 90.9 mmol). The reaction was stirred at room temperature for 3 h and then concentrated from methylene chloride (2×) and diethyl ether (2×). The resulting solid was dissolved in 1:1 MeCN:H₂O and lyophilized overnight to provide 2-((5-((4-bromo-2-fluorophenyl)amino)-4-fluoro-1-methyl-1H-benzo[d]imidazole-6-carboxamido)oxy)acetic acid (H11) (815 mg, 1.25 mmol, 76.3%, TFA salt) as a yellow solid. ¹H NMR (500 MHz, DMSO-d₆) δ 11.83 (s, 1H), 8.46 (s, 1H), 7.84 (s, 1H), 7.76 (s, 1H), 7.43 (dd, J=11.1, 2.2 Hz, 1H), 7.10 (dd, J=8.7, 2.2 Hz, 1H), 6.39 (td, J=9.0, 2.9 Hz, 1H), 4.41 (s, 2H), 3.90 (s, 3H). ¹⁹F NMR (471 MHz, DMSO-d₆) δ −75.37, −130.17, −133.57. LCMS: C₁₇H₁₃BrF₂N₄O₄ requires: 454.0, found: m/z=455.2 [M+H]⁺.

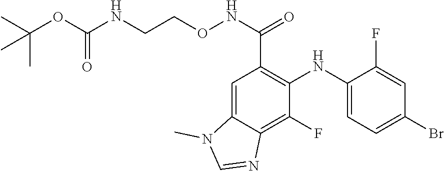 [SMILES: Cn1cnc2c(F)c(Nc3ccc(Br)cc3F)c(C(=O)NOCCNC(=O)OC(C)(C)C)cc21]

### Synthesis of tert-butyl (2-((5-((4-bromo-2-fluorophenyl)amino)-4-fluoro-1-methyl-1H-benzo[d]imidazole-6-carboxamido)oxy)ethyl)carbamate

5-((4-bromo-2-fluorophenyl)amino)-4-fluoro-1-methyl-1H-benzo[d]imidazole-6-carboxylic acid (764.3 mg, 2.000 mmol), tert-butyl N-[2-(aminooxy)ethyl]carbamate (396.4 mg, 2.249 mmol), ({[3-(dimethylamino)propyl]imino}methylidene)(ethyl)amine hydrochloride (421.5 mg, 2.199 mmol), and hydroxybenzotriazole hydrate (167.0 mg, 1.090 mmol) were dissolved in DMF (5.00 mL). triethylamine (0.84 mL, 0.61 g, 6.0 mmol) was added and the reaction was stirred at room temperature overnight. The reaction was concentrated and diluted with ethyl acetate and brine. The aqueous phase was extracted with ethyl acetate (3×) and the combined organic phases were dried over sodium sulfate, filtered, concentrated, and purified by flash chromatography on a 24 g silica column, eluted by gradient elution with 0 to 20% MeOH/EtOAc to provide tert-butyl (2-((5-((4-bromo-2-fluorophenyl)amino)-4-fluoro-1-methyl-1H-benzo[d]imidazole-6-carboxamido)oxy)ethyl)carbamate (600 mg, 55.52%) as a yellow solid. LCMS: C₂₂H₂₄BrF₂N₅O₄ requires: 541.1, found: m/z=542.4 [M+H]⁺.

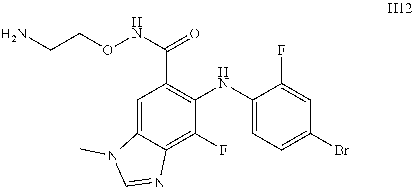 [SMILES: Cn1cnc2c(F)c(Nc3ccc(Br)cc3F)c(C(=O)NOCCN)cc21]

### Synthesis of H12: N-(2-aminoethoxy)-5-((4-bromo-2-fluorophenyl)amino)-4-fluoro-1-methyl-1H-benzo[d]imidazole-6-carboxamide (H12)

To a stirred solution of tert-butyl (2-((5-((4-bromo-2-fluorophenyl)amino)-4-fluoro-1-methyl-1H-benzo[d]imidazole-6-carboxamido)oxy)ethyl)carbamate (1008.0 mg, 1.8654 mmol) in methylene chloride (35.3 mL, 46.6 g, 548 mmol) was added trifluoroacetic acid (8.82 mL, 13.0 g, 114 mmol). The reaction was stirred at room temperature for 2 h and then concentrated from methylene chloride (2×) and diethyl ether (2×). The resulting solid was dissolved in 1:1 MeCN:H₂O and lyophilized overnight to provide N-(2-aminoethoxy)-6-[(4-bromo-2-fluorophenyl)amino]-7-fluoro-3-methyl-1,3-benzodiazole-5-carboxamide (990.5 mg, 1.79 mmol, 91.4%, TFA salt) as a yellow solid. ¹H NMR (500 MHz, DMSO-d₆) δ 11.92 (s, 1H), 8.42 (s, 1H), 7.92-7.85 (m, 2H), 7.77 (s, 1H), 7.75 (s, 1H), 7.43 (dd, J=11.3, 2.2 Hz, 1H), 7.13-7.07 (m, 1H), 6.38 (t, J=8.7 Hz, 1H), 4.01 (t, J=4.9 Hz, 2H), 3.91 (s, 3H), 3.03 (d, J=7.8 Hz, 2H). ¹⁹F NMR (471 MHz, DMSO-d₆) δ −74.23, −130.30, −133.57. LCMS: C₁₇H₁₆BrF₂N₅O₂ requires: 441.0, found: m/z=442.2 [M+H]⁺.

### Example 89: Hook for BCR/ABL

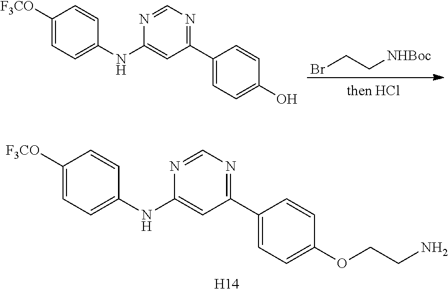 [SMILES: Oc1ccc(-c2cc(Nc3ccc(OC(F)(F)F)cc3)ncn2)cc1 ; CC(C)(C)OC(=O)NCCBr ; NCCOc1ccc(-c2cc(Nc3ccc(OC(F)(F)F)cc3)ncn2)cc1 ; "H14"]

Procedure: 4-(6-{[4-(trifluoromethoxy)phenyl]amino}pyrimidin-4-yl)phenol (1.26 g, 3.63 mmol, Enamine catalog number EN300-28221232), tert-butyl N-(2-bromoethyl)carbamate (2439 mg, 10.88 mmol), and cesium carbonate (3546 mg, 10.88 mmol) were charged to a 20 mL microwave vial followed by 1,4-dioxane (10 mL). The vial was sealed and heated in a microwave reactor to 125 degrees Celsius for 1.5 hours. The mixture was filtered then concentrated. The crude was redissolved in DCM (7 mL). trifluoroacetic acid (6.94 mL, 90.7 mmol) was added and the mixture was stirred at room temperature for 15 minutes. The mixture was concentrated. The crude was purified on a 100 g C18Aq isco column (gradient 0-100% ACN-0.1% TFA: water-0.1% TFA). Desired fractions were concentrated then lyophilized to yield 6-[4-(2-aminoethoxy)phenyl]-N-[4-(trifluoromethoxy)phenyl]pyrimidin-4-amine (H₁₄) (1.353 g, 53.54%) as the TFA salt. M+H⁺ m/z expected=391.14, found=391.34 ¹H NMR (500 MHz, DMSO) δ 10.22 (s, 1H), 8.76 (d, J=1.0 Hz, 1H), 8.12-7.97 (m, 5H), 7.87-7.77 (m, 2H), 7.38 (d, J=8.6 Hz, 2H), 7.24 (d, J=1.1 Hz, 1H), 7.20-7.11 (m, 2H), 4.26 (t, J=5.0 Hz, 2H), 3.28 (q, J=5.4 Hz, 2H).

### Example 90: Hooks for MALT

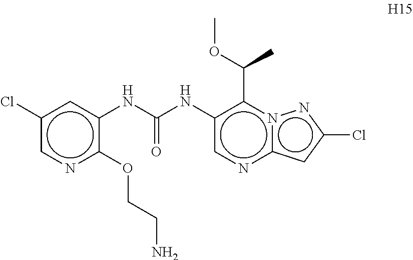

### (S)-1-(2-(2-aminoethoxy)-5-chloropyridin-3-yl)-3-(2-chloro-7-(1-methoxyethyl)pyrazolo[1,5-a]pyrimidin-6-yl)urea (H15) was Purchased from PharmaBlock

### Synthesis of 1-(5-chloro-6-(2H-1,2,3-triazol-2-yl)pyridin-3-yl)-3-(2-chloro-7-(1-(methylamino)ethyl)pyrazolo[1,5-a]pyrimidin-6-yl)urea

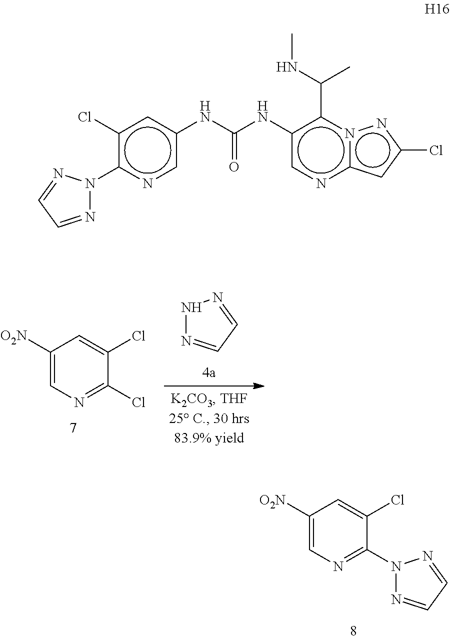

To a mixture of Cpd_7 (22.0 g, 114 mmol, 1.00 eq) and K₂CO₃ (31.5 g, 228 mmol, 2.00 eq) in THF (220 mL) was added Cpd_4a (9.45 g, 136 mmol, 7.94 mL, 1.20 eq) and the reaction mixture was stirred at 25° C. for 30 hours. LCMS showed that Cpd 7 consumed and the desired mass (RT=0.644 min) was given. The reaction mixture was poured into H₂O (300 mL), then was extracted with ethyl acetate (300 mL*3). The combined organic layer was washed with brine (100 mL*2), dried over Na₂SO₄, filtered and concentrated. The residue was treated with ethyl acetate (50.0 mL) at 25° C. for 1 hr, then filtered, and the filter cake was concentrated vacuum. Cpd_8 (21.7 g, 95.7 mmol, 83.9% yield, 99.5% purity) was obtained as a white solid. LCMS: M+H+ m/z expected=225, found=226. ¹H NMR: (400 MHz, CDCl₃) δ 9.32 (s, 1H), 8.78 (d, J=2.4 Hz, 1H), 8.03 (s, 2H).

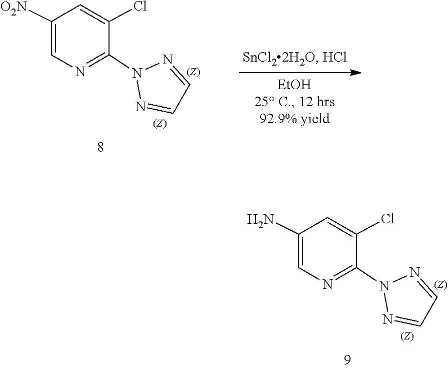 [SMILES: O=[N+]([O-])c1cnc(-n2nccn2)c(Cl)c1 ; Nc1cnc(-n2nccn2)c(Cl)c1]

To a solution of Cpd_8 (19.7 g, 86.8 mmol, 99.5% purity, 1.00 eq) in HCl (100 mL) and EtOH (400 mL) was added SnCl₂.2H₂O (98.0 g, 434 mmol, 5.00 eq), then reaction mixture was added 25° C. for 12 hrs. LCMS showed that Cpd_8 consumed and the desired mass (RT=0.310 min) was given. The reaction mixture was concentrated under vacuum. The residue was adjusted to pH=10 with 3N NaOH solution, then was extracted with ethyl acetate (500 mL*3). The combined organic layer was washed with brine (500 mL*2), dried over Na₂SO₄, filtered and concentrated. Cpd_9 (17.5 g, 88.8 mmol, 92.9% yield, 99.3% purity) was obtained as a yellow solid. LCMS: M+H+ m/z expected=195, found=196. ¹H NMR: (400 MHz, CDCl₃) δ 7.85 (s, 1H), 7.81 (d, J=2.8 Hz, 2H), 7.08 (d, J=2.4 Hz, 1H), 4.28 (s, 2H).

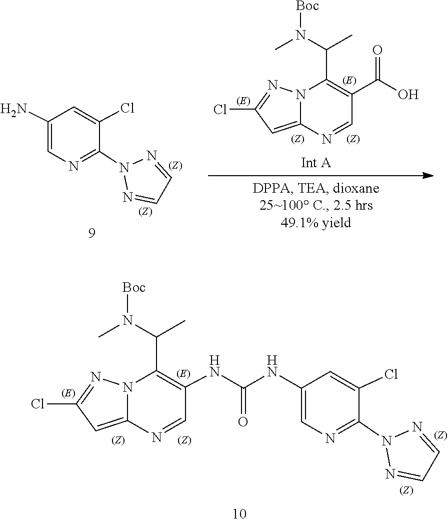 [SMILES: Nc1cnc(-n2nccn2)c(Cl)c1 ; CC(c1c(C(=O)O)cnc2cc(Cl)nn12)N(C)C(=O)OC(C)(C)C ; CC(c1c(NC(=O)Nc2cnc(-n3nccn3)c(Cl)c2)cnc2cc(Cl)nn12)N(C)C(=O)OC(C)(C)C]

DPPA (6.81 g, 24.7 mmol, 5.36 mL, 1.20 eq) and TEA (7.83 g, 77.3 mmol, 10.7 mL, 3.75 eq) were added to a solution of Int A (7.50 g, 20.6 mmol, 97.6% purity, 1.00 eq) in dioxane (75.0 mL). The resulting yellow solution was stirred at 25° C. for 30 minutes. Cpd_9 (4.06 g, 20.6 mmol, 99.3% purity, 1.00 eq) was added and the reaction mixture was heated to 100° C. and stirred for 2 hrs. LCMS showed that Cpd_9 remained (RT=0.395 min) and the desired mass (RT=0.943 min) was given. The reaction mixture with EW34522-31 was poured into H₂O (100 mL), then was extracted with ethyl acetate (100 mL*3). The combined organic layer was washed with brine (100 mL*2), dried over Na₂SO₄, filtered and concentrated. The crude product was purified by Prep-HPLC (basic condition), then concentrated by lyophilization. Cpd_10 (6.50 g, 11.4 mmol, 49.1% yield, 96.5% purity) was obtained as a yellow solid. LCMS: M+H+ m/z expected=546, found=547. ¹H NMR: (400 MHz, CDCl₃) δ 8.91 (s, 1H), 8.80 (s, 1H), 8.52 (d, J=1.2 Hz, 1H), 8.42 (s, 1H), 8.29 (s, 1H), 7.93 (s, 2H), 6.66 (s, 1H), 5.62-5.65 (m, 1H), 3.24 (s, 3H), 1.87 (d, J=7.2 Hz, 1H), 1.40 (s, 9H)

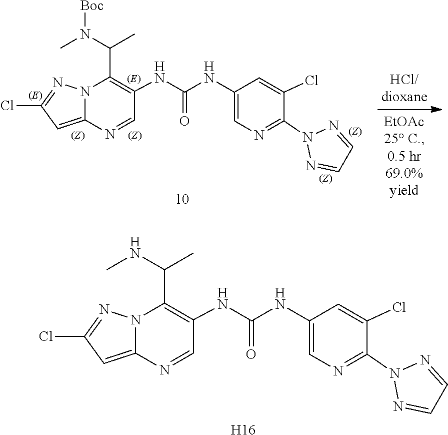 [SMILES: CC(c1c(NC(=O)Nc2cnc(-n3nccn3)c(Cl)c2)cnc2cc(Cl)nn12)N(C)C(=O)OC(C)(C)C ; CNC(C)c1c(NC(=O)Nc2cnc(-n3nccn3)c(Cl)c2)cnc2cc(Cl)nn12 ; "H16"]

To a solution of Cpd_10 (6.50 g, 11.2 mmol, 94.9% purity, 1.00 eq) in EtOAc (60.0 mL) was added HCl/dioxane (4.00 M, 100 mL, 35.5 eq), then reaction mixture was stirred at 25° C. for 0.5 hr. LCMS showed that Cpd_10 consumed, and the desired mass (RT=0.667 min) was given. The reaction mixture was concentrated under vacuum. The residue was treated with ethyl acetate (10.0 mL) at 25° C. for 1 hr, then filtered, and the filter cake was concentrated vacuum. H16 (3.50 g, 7.78 mmol, 69.0% yield, 99.4% purity) was obtained as a white solid. LCMS: M+H+ m/z expected=446, found=447. ¹H NMR: (400 MHz, CDCl₃) δ 8.67 (s, 1H), 8.63 (d, J=2.0 Hz, 1H), 8.50 (d, J=2.4 Hz, 1H), 8.04 (s, 2H), 6.91 (s, 1H), 5.05-5.10 (m, 1H), 2.80 (s, 3H), 1.95 (d, J=6.8 Hz, 3H)

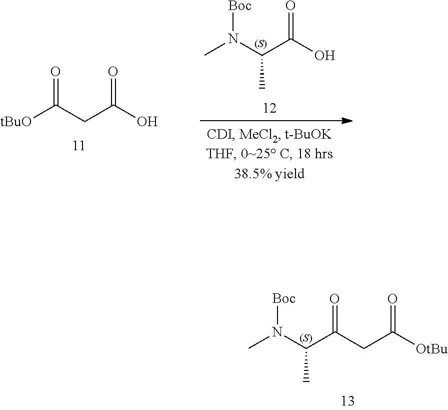

Flask A: t-BuOK (1.00 M, 217 mL, 1.20 eq) was added dropwise to a solution of Cpd_11 (34.8 g, 217 mmol, 33.4 mL, 1.20 eq) and MgCl₂ (20.7 g, 217 mmol, 8.92 mL, 1.20 eq) in THF (400 mL) at 0° C., the reaction mixture was stirred at 25° C. for 3 hrs. Flask B: CDI (35.2 g, 217 mmol, 1.20 eq) was added to a solution of Cpd_12 (36.8 g, 181 mmol, 1.00 eq) in THF (400 mL) and the reaction mixture was stirred at 25° C. for 3 hrs. The contents in flask B were added via an additional funnel to flask A, and the resulting cloudy white mixture was stirred for 12 hrs at 25° C. LCMS showed that Cpd_11 consumed, and the desired mass (RT=0.951 min) was given. The reaction mixture was poured into H₂O (500 mL), then was extracted with ethyl acetate (500 mL*3). The combined organic layer was washed with brine (500 mL*2), dried over Na₂SO₄, filtered and concentrated. The residue was purified by column chromatography (SiO₂, Petroleum ether/ethyl acetate=50/1-25/1-10/1). The product was given (Petroleum ether/Ethyl acetate=5/1, R_{f}=0.47). Cpd_13 (25.0 g, 69.8 mmol, 38.5% yield, 84.2% purity) was obtained as a yellow oil. LCMS: M-100-56+H+m/z expected=145, found=146. ¹H NMR: (400 MHz, CDCl₃) δ 4.56-4.59 (m, 0.5H), 4.08-4.13 (m, 0.5H), 3.42-3.50 (m, 1H), 3.31 (d, J=16.0 Hz, 1H), 2.80 (d, J=48.4 Hz, 2H), 1.44 (s, 18H), 1.28 (s, 3H).

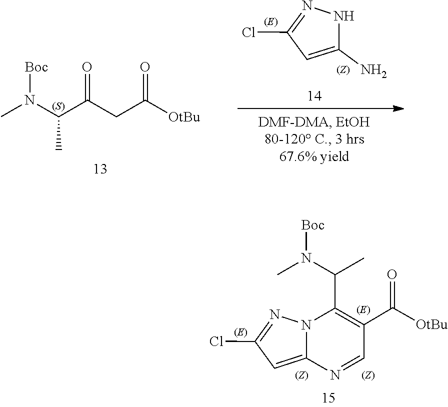

A solution of Cpd_13 (17.5 g, 48.9 mmol, 84.2% purity, 1.00 eq) in DMF-DMA (15.0 g, 126 mmol, 16.7 mL, 2.58 eq) was heated at 120° C. for 1 hr, then cooled to 80° C., and a solution of Cpd_14 (5.75 g, 48.9 mmol, 1.00 eq) in EtOH (60.0 mL) was added to the reaction mixture. The resulting mixture was stirred for 2 hrs at 80° C. LCMS showed that Cpd_13 consumed, and the desired mass (RT=1.022 mins) was given. The reaction mixture was poured into H₂O (100 mL), then was extracted with ethyl acetate (100 mL*3). The combined organic layer was washed with brine (100 mL*2), dried over Na₂SO₄, filtered and concentrated. The residue was purified by column chromatography (SiO₂, Petroleum ether/ethyl acetate=100/1-50/1-20/1). The product was given (Petroleum ether/Ethyl acetate=5/1, R_{f}=0.65). Cpd_15 (14.0 g, 33.0 mmol, 67.6% yield, 97.1% purity) was obtained as a white solid. LCMS: M-56+H+ m/z expected=354, found=355. ¹H NMR: (400 MHz, DMSO d₆) δ 8.72 (s, 1H), 7.02 (s, 1H), 5.77 (d, J=5.6 Hz, 1H), 2.94 (s, 3H), 1.67 (d, J=7.6 Hz, 3H), 1.54 (s, 9H), 1.33 (s, 3H), 0.83 (s, 6H)

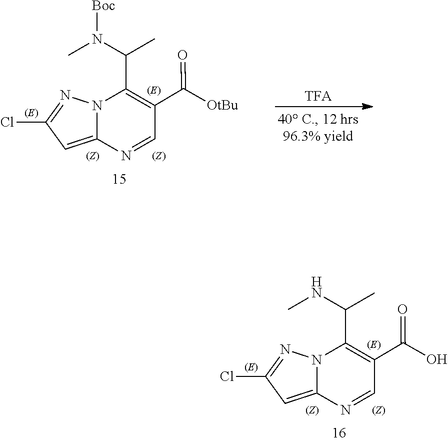

To a solution of Cpd_15 (14.0 g, 33.0 mmol, 97.1% purity, 1.00 eq) in TFA (84.4 g, 740 mmol, 54.8 mL, 22.4 eq) was stirred at 40° C. for 12 hrs. LCMS showed that Cpd_15 consumed, and the desired mass (RT=0.255 min) was given. The reaction mixture was concentrated under vacuum to give Cpd_16 (8.43 g, 31.8 mmol, 96.3% yield, 96.3% purity) as a yellow solid. LCMS: M+H+ m/z expected=254, found=255

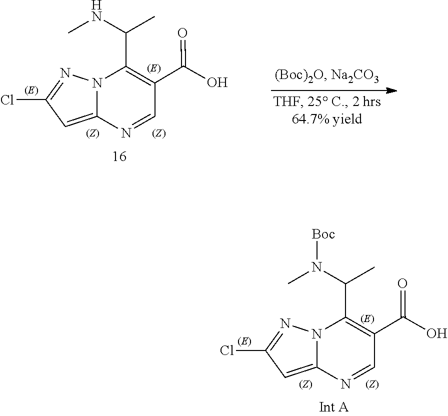

To a solution of Cpd_16 (8.43 g, 31.8 mmol, 96.3% purity, 1.00 eq) and Na₂CO₃ (2.00 M, 31.8 mL, 2.00 eq) in THF (80.0 mL) was added (Boc)₂O (8.35 g, 38.2 mmol, 8.79 mL, 1.20 eq) at 25° C. Then reaction mixture was stirred 25° C. for 2 hrs. LCMS showed that Cpd_16 consumed, and the desired mass (RT=0.848 min) was given. The reaction mixture was poured into H₂O (10.0 mL), then was extracted with ethyl acetate (10.0 mL*3). The aqueous phase was adjusted to pH=4˜5 with 1N aqueous citric acid solution and extracted with ethyl acetate (10.0 mL*3). The combined organic layer was washed with brine (10.0 mL*2), dried over Na₂SO₄, filtered and concentrated. Int A (7.50 g, 20.6 mmol, 64.7% yield, 97.6% purity) was obtained as a yellow solid. LCMS: M-56+H+ m/z expected=298, found=299. ¹H NMR: (400 MHz, DMSO d₆) δ 8.80 (s, 1H), 7.02 (s, 1H), 6.00 (d, J=6.4 Hz, 1H), 2.99 (s, 3H), 1.66 (d, J=7.2 Hz, 3H), 1.26 (s, 3H), 0.88 (s, 6H)

### Example 91: Hook for Pan Kinase Binder

### N-(2-chloro-6-methylphenyl)-2-((2-methyl-6-(piperazin-1-yl)pyrimidin-4-yl)amino)thiazole-5-carboxamide (H17)

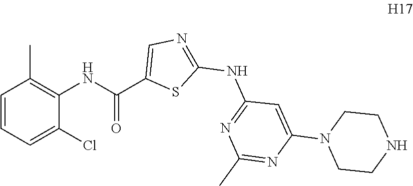 [SMILES: Cc1nc(Nc2ncc(C(=O)Nc3c(C)cccc3Cl)s2)cc(N2CCNCC2)n1]

H17 was purchased from supplier: MedChemExpress

### Example 92: Hook for IRAK 1

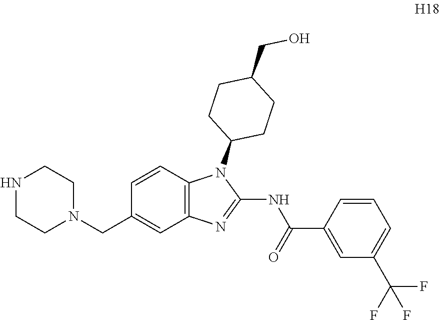 [SMILES: O=C(Nc1nc2cc(CN3CCNCC3)ccc2n1[C@H]1CC[C@@H](CO)CC1)c1cccc(C(F)(F)F)c1]

N-(1-((1s,4s)-4-(hydroxymethyl)cyclohexyl)-5-(piperazin-1-ylmethyl)-1H-benzo[d]imidazol-2-yl)-3-(trifluoromethyl)benzamide (H18) was synthesized from (4-fluoro-3-nitro-phenyl)methanol according to WO2019099926, which is incorporated by reference in its entirety.

### Example 93: Hook for Pan Kinase Binder

### 2-((5-chloro-2-((4-(piperazin-1-yl)phenyl)amino)pyrimidin-4-yl)amino)-N-methylbenzamide (H19)

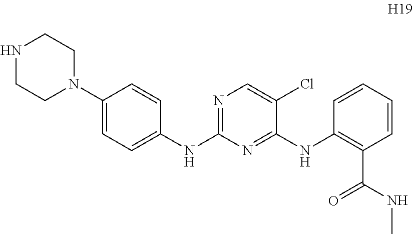 [SMILES: CNC(=O)c1ccccc1Nc1nc(Nc2ccc(N3CCNCC3)cc2)ncc1Cl]

H19 was purchased from Aurum Pharmtech.

### Example 94: Hook for Pan Kinase Binder

### N-(3-fluoro-4-((6-methoxy-7-(piperidin-4-ylmethoxy)quinolin-4-yl)oxy)phenyl)-N-(4-fluorophenyl)cyclopropane-1,1-dicarboxamide (H20)

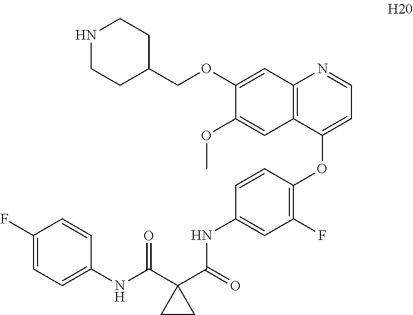 [SMILES: COc1cc2c(Oc3ccc(NC(=O)C4(C(=O)Nc5ccc(F)cc5)CC4)cc3F)ccnc2cc1OCC1CCNCC1]

H20 was synthesized according to reference: WO2012100459 A1, which is incorporated by reference in its entirety

### Example 95: Generic Methodology for Compound Parallel Synthesis

Compounds 349-485 were prepared consistent with the following methods.

Target molecules are identified by tag chains (TagIDs). TagIDs consist of individual components identifiers (codes) linked together in the order of addition during synthesis.

Target compounds are synthesized in parallel using a convergent approach:

R1+R2 [symbol] R1-R2

R3+R4 [symbol] R3-R4

R1−R2+R3-R4 [symbol] R1-R2-R3-R4

R1+R5 [symbol] R1-R5

R1+R3-R4 [symbol] R1-R3-R4

Pairs R1-2 and R3-R4 are either synthesized in a large vessel then transferred in 2 mL FluidX tubes or directly synthesized in 2 mL FluidX tubes. Each R1 is a harness coded as Har1, Har2, Har3, Har4, or Har 5. Each R2 is a boc di-amine coded as 0147, 0172, or 0226. Each carboxylic tButyl ester (0212) R3 is a boc di-Amine codded as 0117 or 0147; a boc amino acid coded as 0225; an amino acid t-butyl ester coded as 0212; or a mono t-Butyl ester dicarboxylic acid coded as 0173. Each R4 is a hook coded as H01 to H21. Each R5 is an amine-containing hook coded as H01-H05, H09-H10, or H12-H20. Reactions are carried out on in 2 mL FluidX tubes.

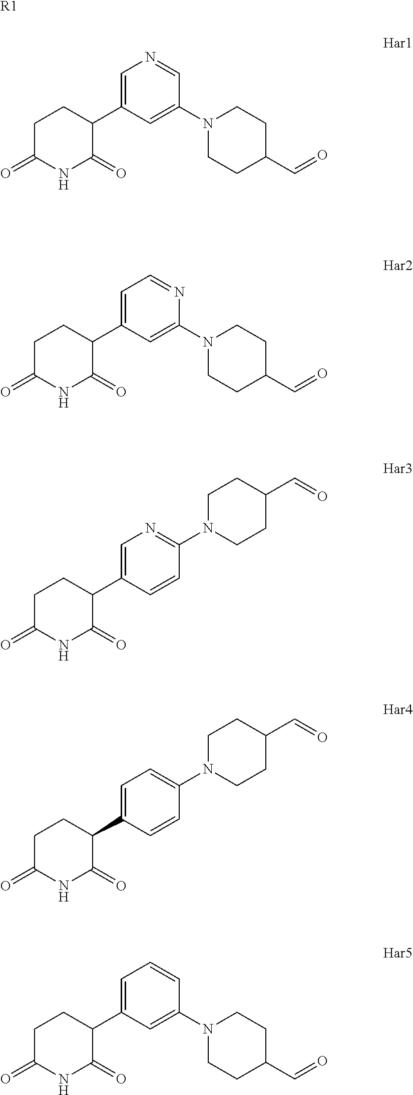

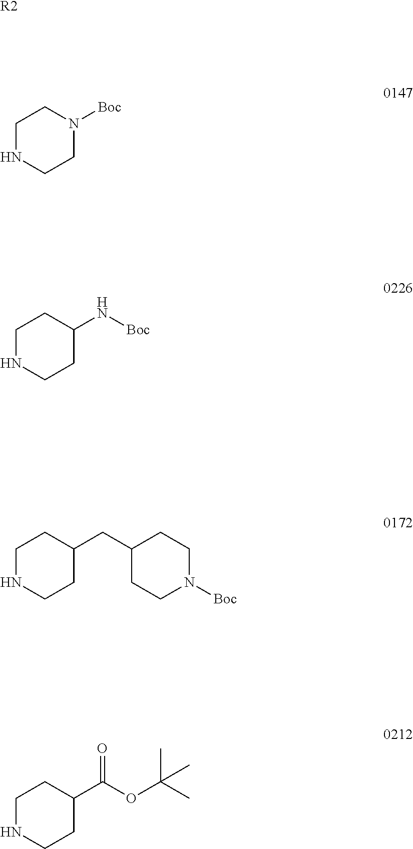

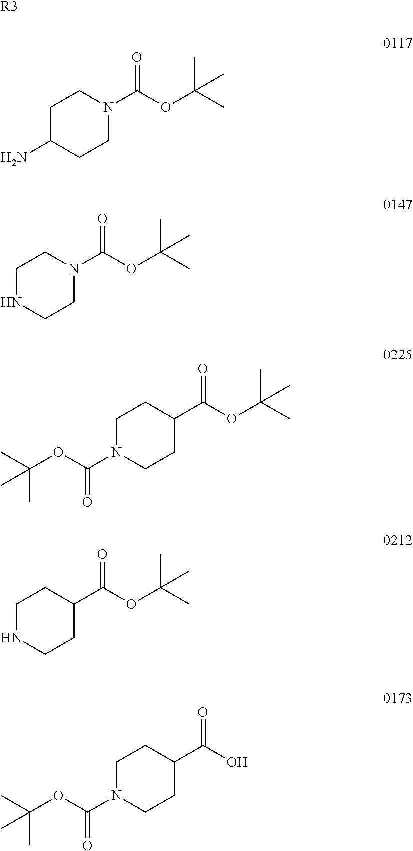

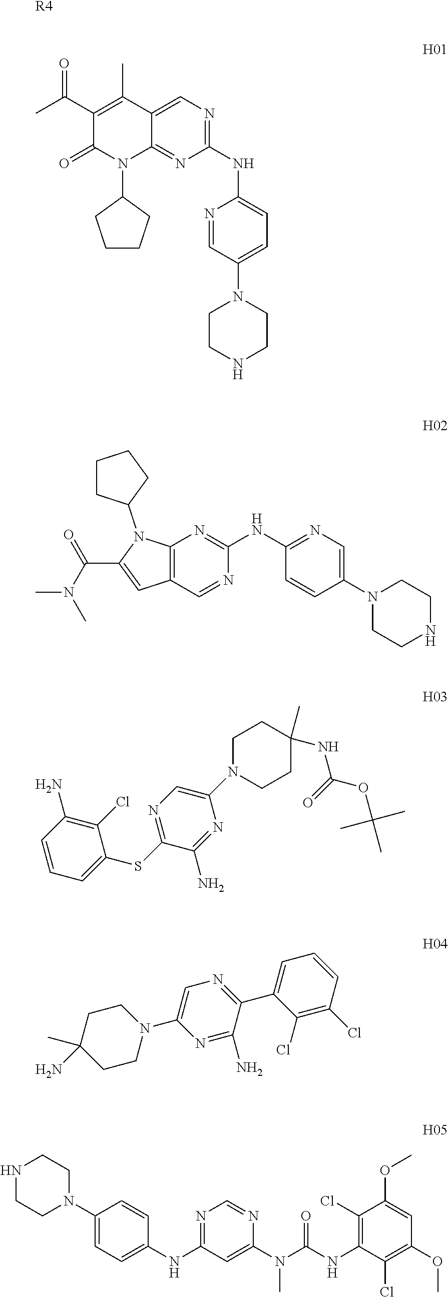

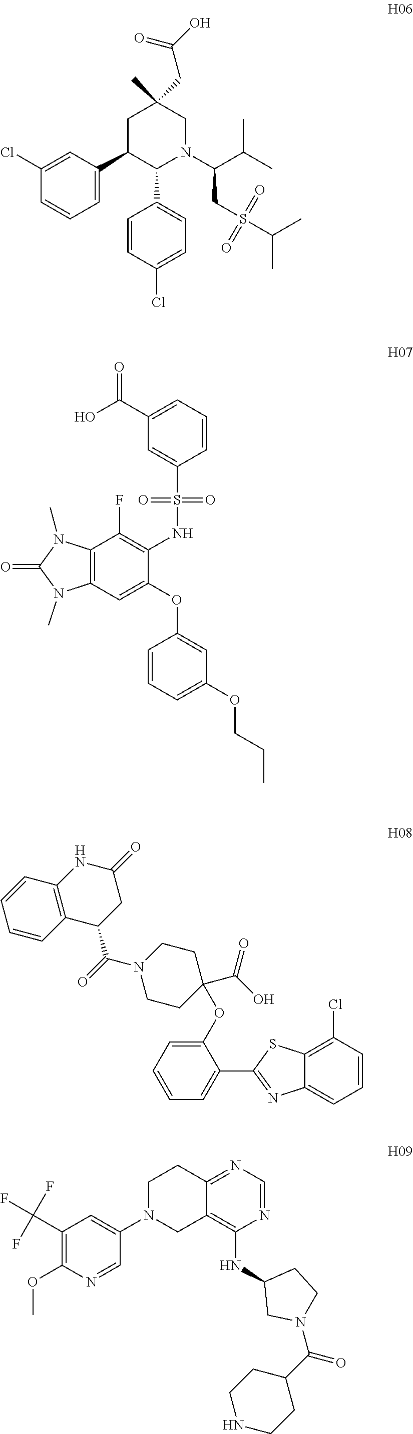 [SMILES: CC(C)[C@@H](CS(=O)(=O)C(C)C)N1C[C@@](C)(CC(=O)O)C[C@H](c2cccc(Cl)c2)[C@H]1c1ccc(Cl)cc1 ; CCCOc1cccc(Oc2cc3c(c(F)c2NS(=O)(=O)c2cccc(C(=O)O)c2)n(C)c(=O)n3C)c1 ; O=C1C[C@H](C(=O)N2CCC(Oc3ccccc3-c3nc4cccc(Cl)c4s3)(C(=O)O)CC2)c2ccccc2N1 ; COc1ncc(N2CCc3ncnc(N[C@H]4CCN(C(=O)C5CCNCC5)C4)c3C2)cc1C(F)(F)F]

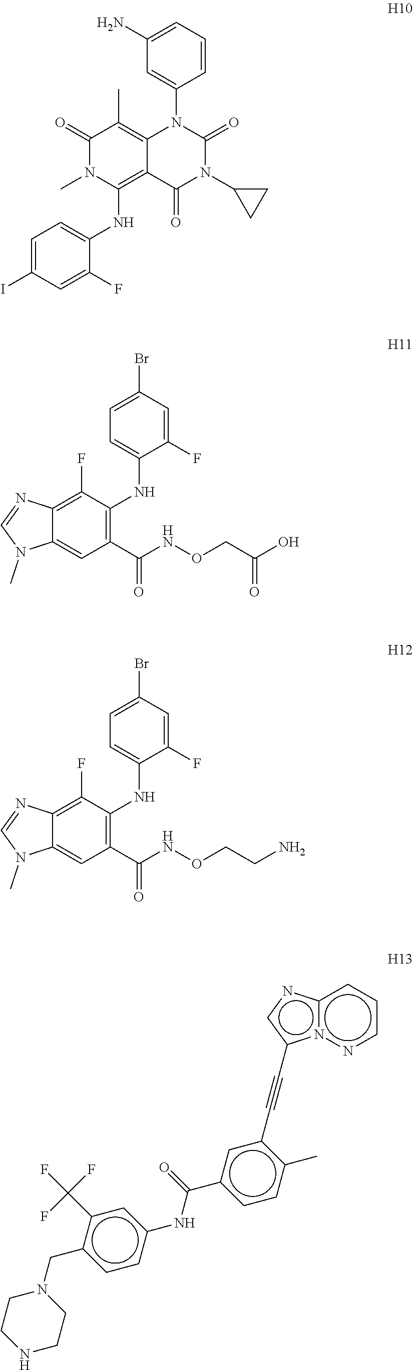

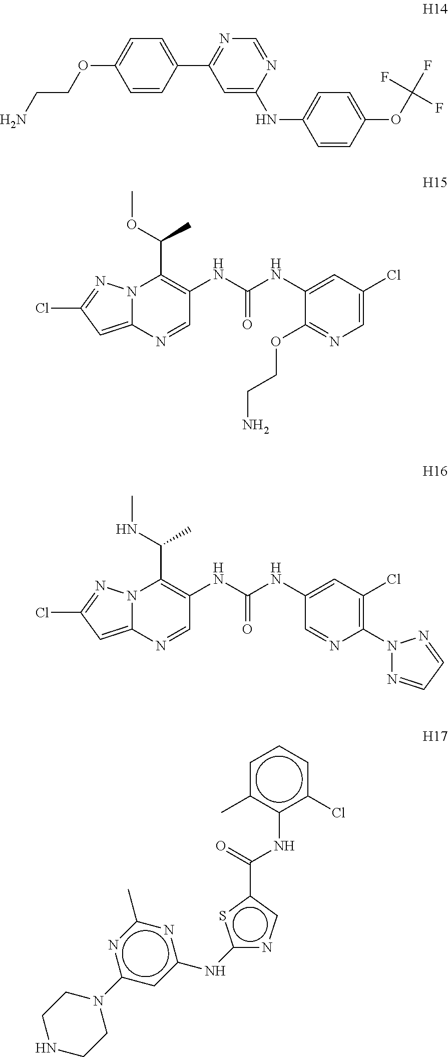

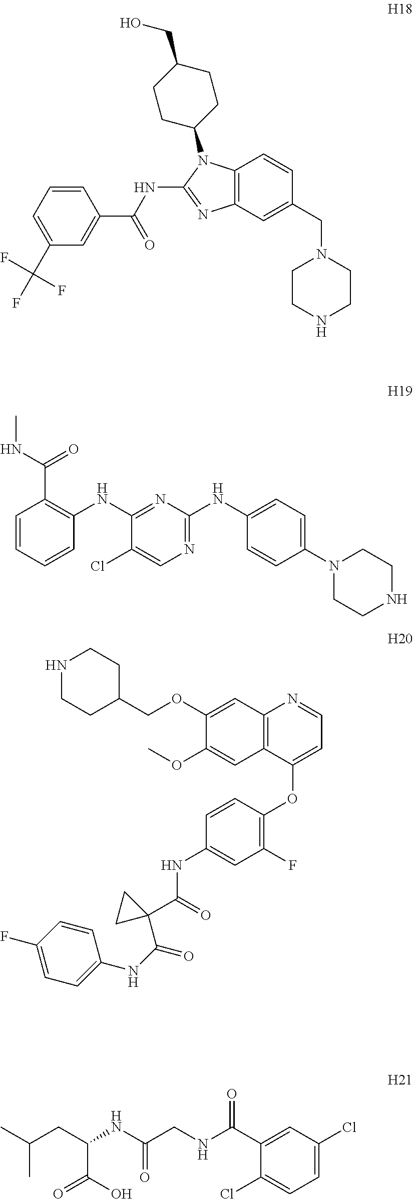 [SMILES: O=C(Nc1nc2cc(CN3CCNCC3)ccc2n1[C@H]1CC[C@@H](CO)CC1)c1cccc(C(F)(F)F)c1 ; CNC(=O)c1ccccc1Nc1nc(Nc2ccc(N3CCNCC3)cc2)ncc1Cl ; COc1cc2c(Oc3ccc(NC(=O)C4(C(=O)Nc5ccc(F)cc5)CC4)cc3F)ccnc2cc1OCC1CCNCC1 ; CC(C)C[C@H](NC(=O)CNC(=O)c1cc(Cl)ccc1Cl)C(=O)O]

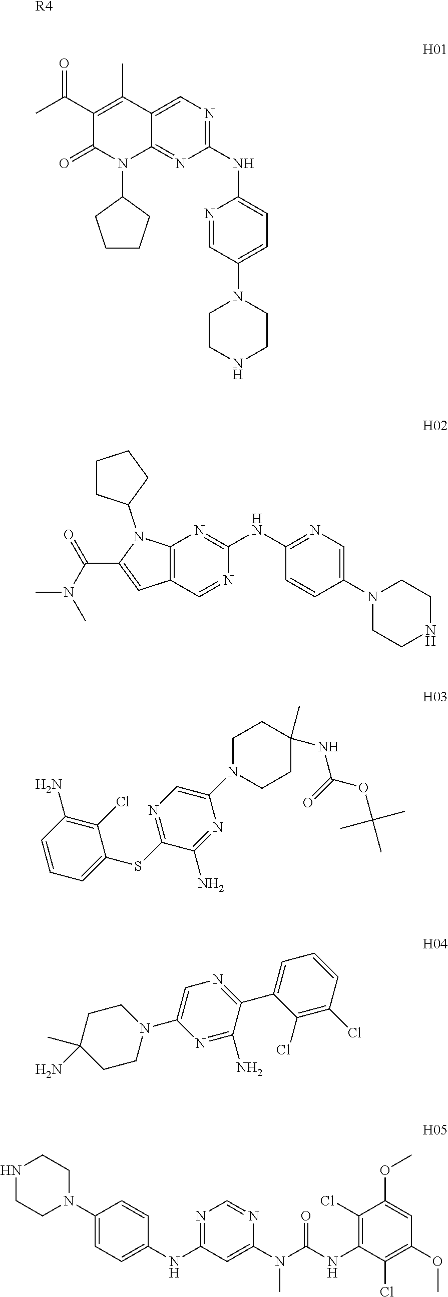

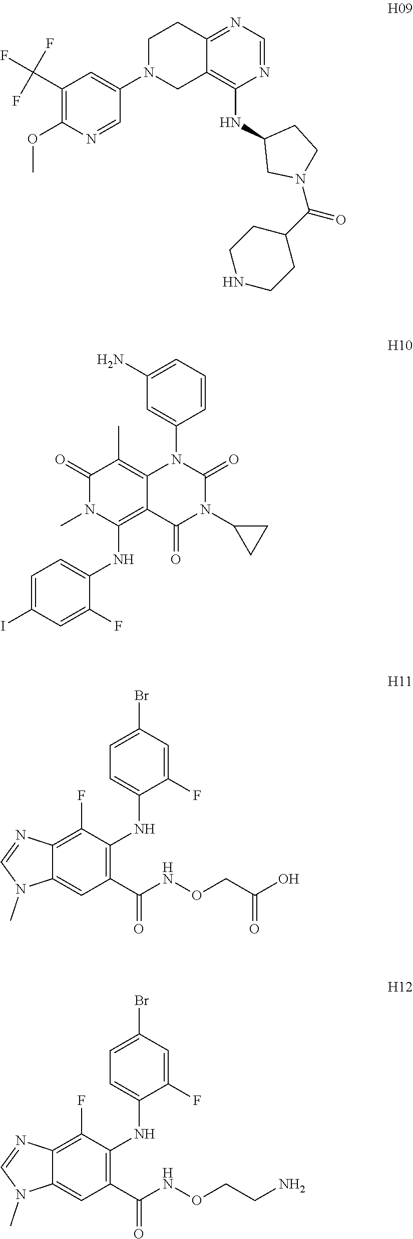 [SMILES: COc1ncc(N2CCc3ncnc(N[C@H]4CCN(C(=O)C5CCNCC5)C4)c3C2)cc1C(F)(F)F ; Cc1c(=O)n(C)c(Nc2ccc(I)cc2F)c2c(=O)n(C3CC3)c(=O)n(-c3cccc(N)c3)c12 ; Cn1cnc2c(F)c(Nc3ccc(Br)cc3F)c(C(=O)NOCC(=O)O)cc21 ; Cn1cnc2c(F)c(Nc3ccc(Br)cc3F)c(C(=O)NOCCN)cc21]

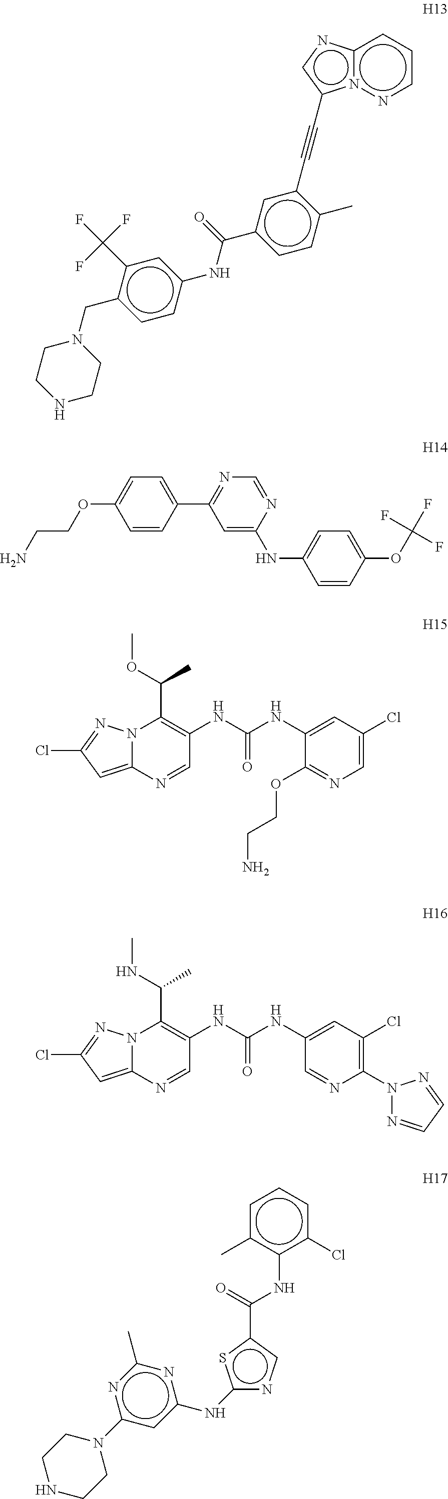

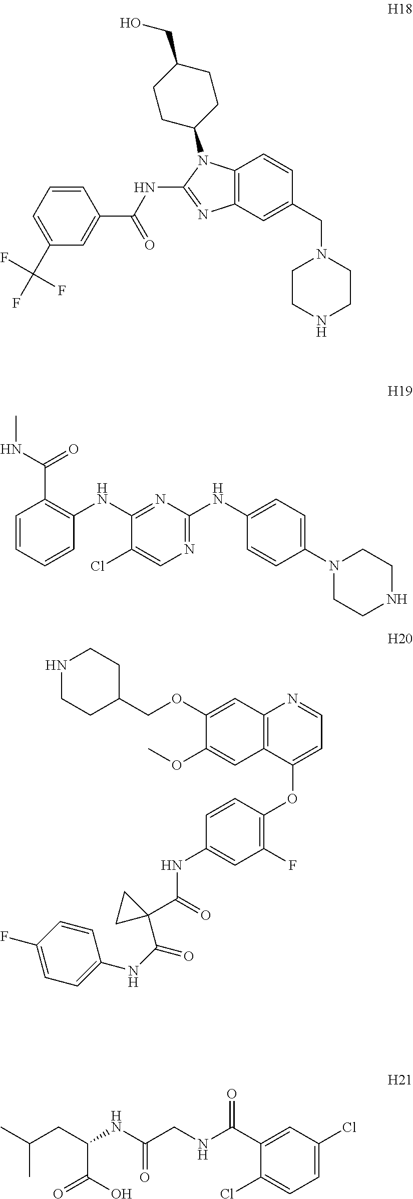 [SMILES: O=C(Nc1nc2cc(CN3CCNCC3)ccc2n1[C@H]1CC[C@@H](CO)CC1)c1cccc(C(F)(F)F)c1 ; CNC(=O)c1ccccc1Nc1nc(Nc2ccc(N3CCNCC3)cc2)ncc1Cl ; COc1cc2c(Oc3ccc(NC(=O)C4(C(=O)Nc5ccc(F)cc5)CC4)cc3F)ccnc2cc1OCC1CCNCC1 ; CC(C)C[C@H](NC(=O)CNC(=O)c1cc(Cl)ccc1Cl)C(=O)O]

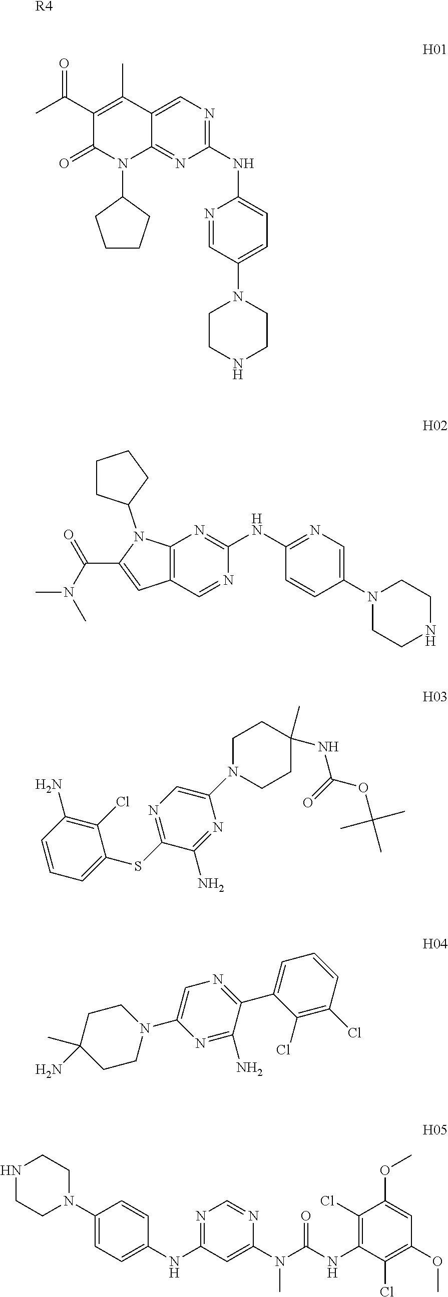

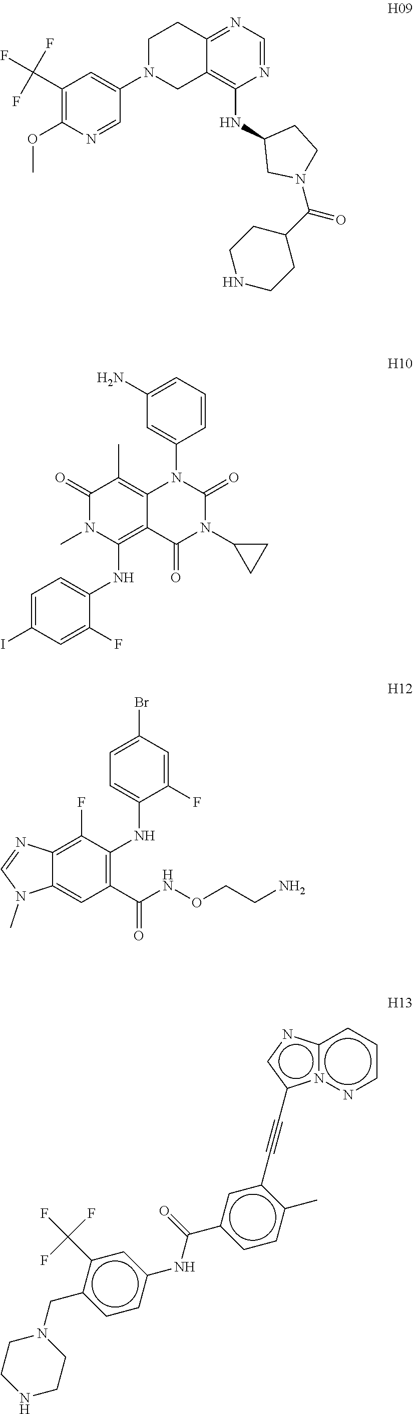

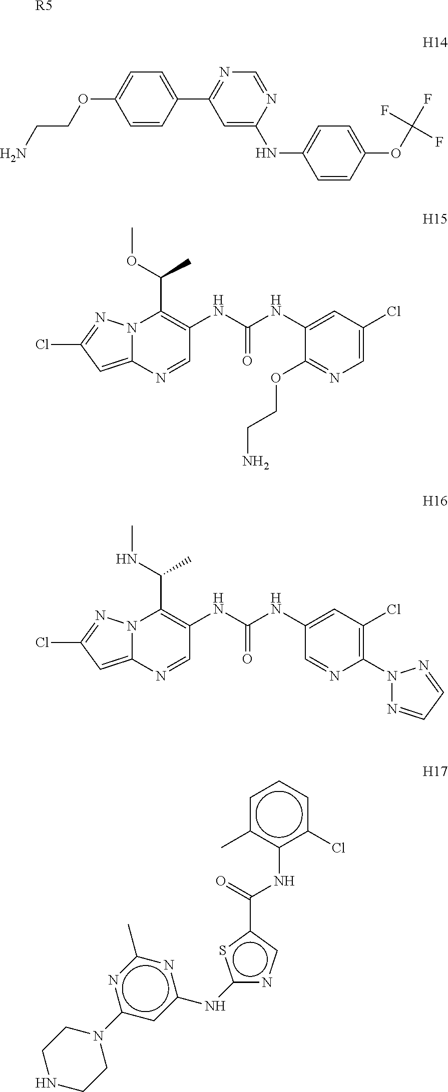

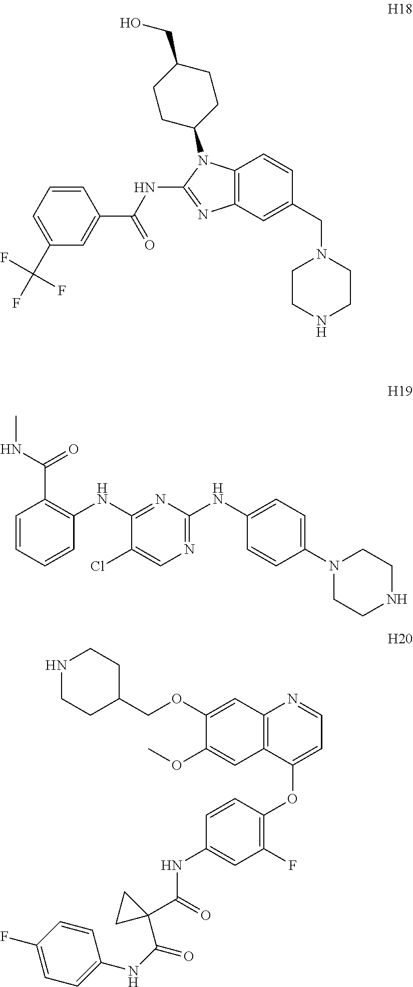 [SMILES: O=C(Nc1nc2cc(CN3CCNCC3)ccc2n1[C@H]1CC[C@@H](CO)CC1)c1cccc(C(F)(F)F)c1 ; CNC(=O)c1ccccc1Nc1nc(Nc2ccc(N3CCNCC3)cc2)ncc1Cl ; COc1cc2c(Oc3ccc(NC(=O)C4(C(=O)Nc5ccc(F)cc5)CC4)cc3F)ccnc2cc1OCC1CCNCC1]

General reductive amination procedure for reactions:

R1+R2 [symbol] R1-R2

R1+R5 [symbol] R1-R5

R1+R3-R4 [symbol] R1-R3-R4

R1-R2, R1-R5, R1-R3-R4 pairs:

To a solution of Aldehyde (10 μmoles) in 100 μL of a 1/1 vol/vol dichloroethane/dimethylformamide mixture, is added successively 100 μL of a solution of amine (11 μmoles, 1.1 eq) in dimethylformamide, and di-isopropyl amine (6 μL, 40 μmoles, 4 eq).

The resulting solution is stirred at room temperature for one hour then transferred into a new container loaded with cyanoborohydride polymer supported (20 mg, 40 μmoles, 4 eq). The reaction is stirred at room temperature for another 8 h after which the solution is transferred into a 96 well filter plate and the solid filtered off. The residue is rinsed twice with 100 μL, of DMF. The combined fractions are dried under reduced pressure to afford a brown oily residue. The crude mixture is used without further purification for the next step. If a Boc or T-butyl ester remains on the compound, the following two methods were utilized for deprotection:

Generic Boc or t-Butyl ester acid deprotection protocol:

The R1−R2 crude mixture from previous step is treated with 400 μL, of a 1/1 vol/vol trifluoroacetic acid/dichloromethane mixture for 2 hours at room temperature. After drying under reduced pressure, the deprotected amine, trifluoroacetic salt, is used without further purification for the next step.

General amide coupling reaction for reactions:

R3+R4 [symbol] R3-R4

R1-R2+R3-R4 [symbol] R1-R2-R3-R4

R3-R4 and R1-R2-R3-R4 pairs.

To a solution of carboxylic acid (10 μmoles) in 100 μL, in dimethylformamide, is added successively 40 μL, of a 1 M solution of diisopropyl amines in DMF (4 eq) and 110 μL, of a 100 mM solution of 1-Bis(dimethylamino)methylene]-1H-1,3-triazolo[4,5-b]pyridinium 3-oxide hexafluorophosphate, Hexafluorophosphate Azabenzotriazole Tetramethyl Uronium (HATU); 1.1 eq and the resulting solution is stirred at room temperature for 20 minutes, after which 100 μL (10 μmoles, 1 eq) of a solution of amine is added to the reaction mixture. The resulting reaction mixture is stirred at room temperature for another 8 hours after which the solvent is removed under reduced pressure to yield a brown oil used without further purification for the next step.

If a Boc or t-butyl ester remains on the compound, the following two methods are utilized for deprotection:

Generic Boc or t-Butyl ester acid deprotection protocol:

The R1-R2 crude mixture from previous step is treated with 400 μL of a 1/1 vol/vol trifluoroacetic acid/dichloromethane mixture for 2 hours at room temperature. After drying under reduced pressure, the deprotected amine, trifluoroacetic salt, is used without further purification for the next step.

All final products are analyzed for desired mass and purified via HPLC.

### Example 96: Compound 352

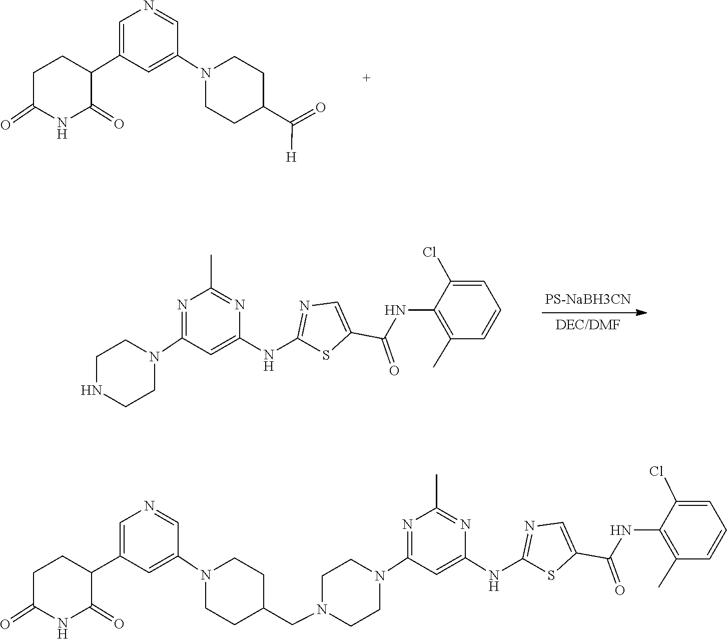

### (S)—N-(2-chloro-6-methylphenyl)-2-((6-(4-((1-(5-(2,6-dioxopiperidin-3-yl)pyridin-3-yl)piperidin-4-yl)methyl)piperazin-1-yl)-2-methylpyrimidin-4-yl)amino)thiazole-5-carboxamide

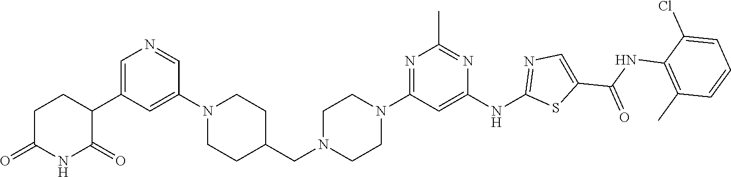 [SMILES: Cc1nc(Nc2ncc(C(=O)Nc3c(C)cccc3Cl)s2)cc(N2CCN(CC3CCN(c4cncc(C5CCC(=O)NC5=O)c4)CC3)CC2)n1]

To a solution of 1-(5-(2,6-dioxopiperidin-3-yl)pyridin-3-yl)piperidine-4-carbaldehyde (3.01 mg, 10 μmoles) in 100 μL of a 1/1 vol/vol Dichloroethane/Dimethylformamide mixture, is added successively 100 μL of a solution of N-(2-chloro-6-methylphenyl)-2-((2-methyl-6-(piperazin-1-yl)pyrimidin-4-yl)amino)thiazole-5-carboxamide (4.9 mg, 11 μmoles, 1.1 eq) in Dimethylformamide, and di-isopropylamine (6 μL, 40 μmoles, 4 eq). The resulting solution is stirred at room temperature for one hour then transferred into a new container loaded with Cyanoborohydride polymer supported (20 mg, 40 μmoles, 4 eq). The reaction is stirred at room temperature for another 8 h after which the solution is transferred into a 96 well filter plate and the solid filtered off. The residue is rinsed twice with 100 μL of DMF. The combined fractions are dried under reduced pressure to afford a brown oily residue. The crude mixture is dissolved in 400 μL of dimethyl formamide and purified by preparative liquid chromatography to yield 1 mg (1.37 μmoles), yield=15%

### Example 97: Compound 354

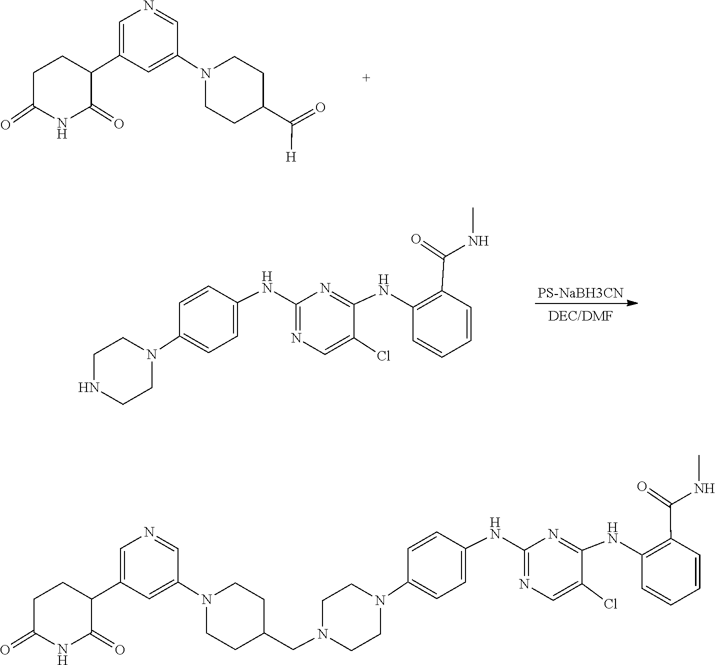

### 2-((5-chloro-2-((4-(4-((1-(5-(2,6-dioxopiperidin-3-yl)pyridin-3-yl)piperidin-4-yl)methyl)piperazin-1-yl)phenyl)amino)pyrimidin-4-yl)amino)-N-methylbenzamide

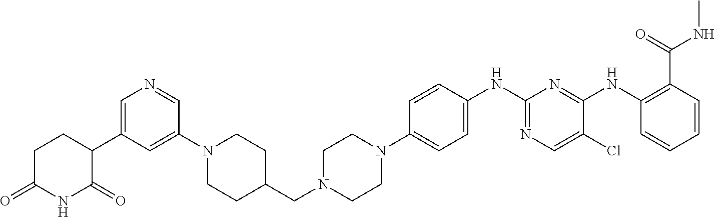 [SMILES: CNC(=O)c1ccccc1Nc1nc(Nc2ccc(N3CCN(CC4CCN(c5cncc(C6CCC(=O)NC6=O)c5)CC4)CC3)cc2)ncc1Cl]

To a solution of 1-(5-(2,6-dioxopiperidin-3-yl)pyridin-3-yl)piperidine-4-carbaldehyde (3.01 mg, 10 μmoles) in 100 μL of a 1/1 vol/vol Dichloroethane/Dimethylformamide mixture, is added successively 100 μL of a solution of 2-((2-((4-(4l2-piperazin-1-yl)phenyl)amino)-5-chloropyrimidin-4-yl)amino)-N-methylbenzamide (4.8 mg, 11 μmoles, 1.1 eq) in Dimethylformamide, and di-isopropylamine (6 μL, 40 μmoles, 4 eq). The resulting solution is stirred at room temperature for one hour then transferred into a new container loaded with Cyanoborohydride polymer supported (20 mg, 40 μmoles, 4 eq). The reaction is stirred at room temperature for another 8 h after which the solution is transferred into a 96 well filter plate and the solid filtered off. The residue is rinsed twice with 100 μL of DMF. The combined fractions are dried under reduced pressure to afford a brown oily residue. The crude mixture is dissolved in 400 μL of dimethyl formamide and purified by preparative liquid chromatography (Method A) to yield 1 mg (1.37 μmoles), yield=14%

### Example 98: Compound 351

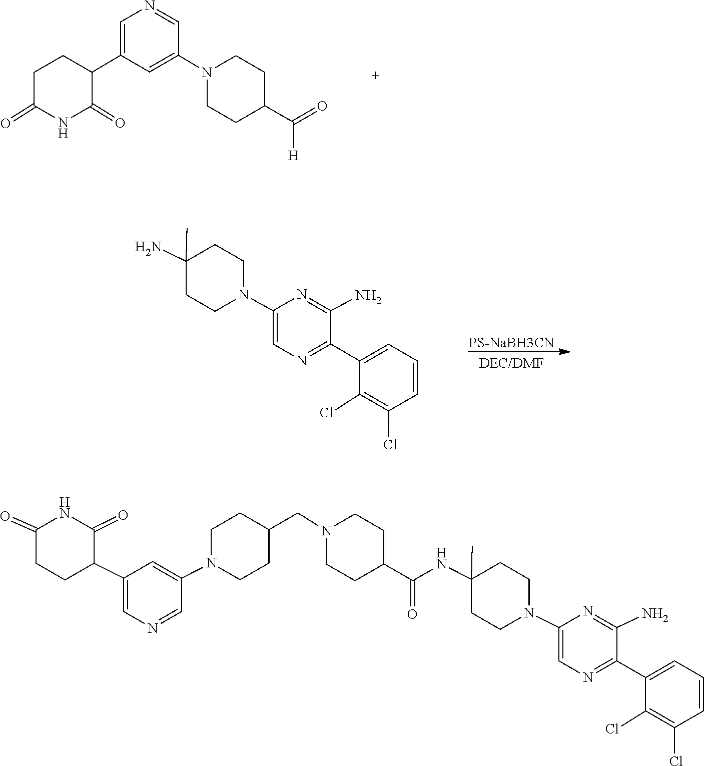 [SMILES: O=CC1CCN(c2cncc(C3CCC(=O)NC3=O)c2)CC1 ; CC1(N)CCN(c2cnc(-c3cccc(Cl)c3Cl)c(N)n2)CC1 ; "PS-NaBH3CN" ; CC1(NC(=O)C2CCN(CC3CCN(c4cncc(C5CCC(=O)NC5=O)c4)CC3)CC2)CCN(c2cnc(-c3cccc(Cl)c3Cl)c(N)n2)CC1]

### N-(1-(6-amino-5-(2,3-dichlorophenyl)pyrazin-2-yl)-4-methylpiperidin-4-yl)-1-((1-(5-(2,6-dioxopiperidin-3-yl)pyridin-3-yl)piperidin-4-yl)methyl)piperidine-4-carboxamide

To a solution of 1-(5-(2,6-dioxopiperidin-3-yl)pyridin-3-yl)piperidine-4-carbaldehyde (3.01 mg, 10 μmoles) in 100 μL of a 1/1 vol/vol Dichloroethane/Dimethylformamide mixture, is added successively 100 μL of a solution of 6-(4-amino-4-methylpiperidin-1-yl)-3-(2,3-dichlorophenyl)pyrazin-2-amine (3.9 mg, 11 μmoles, 1.1 eq) in Dimethylformamide, and di-isopropylamine (6 μL, 40 μmoles, 4 eq). The resulting solution is stirred at room temperature for one hour then transferred into a new container loaded with Cyanoborohydride polymer supported (20 mg, 40 μmoles, 4 eq). The reaction is stirred at room temperature for another 8 h after which the solution is transferred into a 96 well filter plate and the solid filtered off. The residue is rinsed twice with 100 μL of DMF. The combined fractions are dried under reduced pressure to afford a brown oily residue. The crude mixture is dissolved in 400 μL of dimethyl formamide and purified by preparative liquid chromatography (Method A) to yield 1 mg (1.37 μmoles), yield=14%

### Example 99: Compound 469

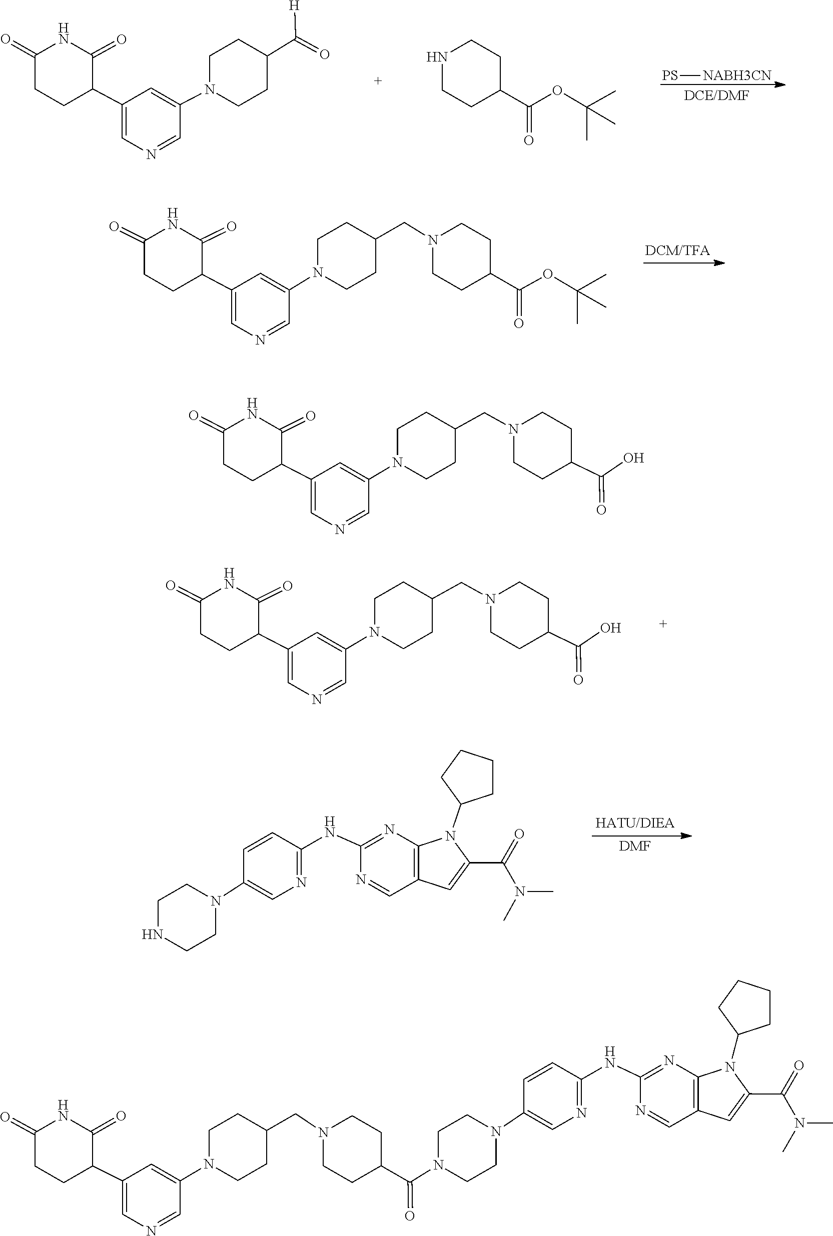

### 1-((1-(5-(2,6-dioxopiperidin-3-yl)pyridin-3-yl)piperidin-4-yl)methyl)piperidine-4-carboxylic acid

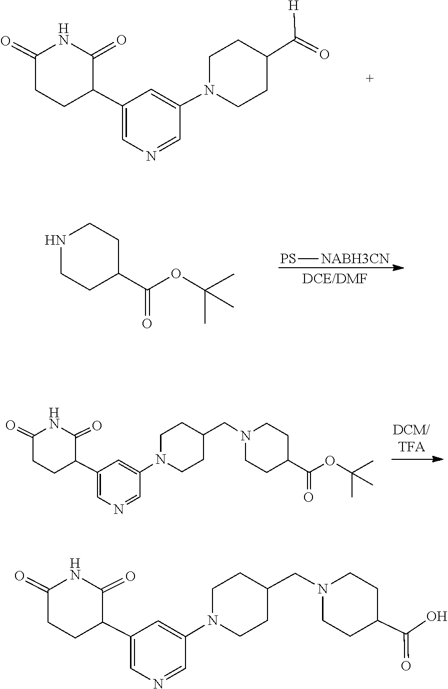

To a solution of 1-(5-(2,6-dioxopiperidin-3-yl)pyridin-3-yl)piperidine-4-carbaldehyde (3.01 mg, 10 μmoles) in 100 μL of a 1/1 vol/vol Dichloroethane/Dimethylformamide mixture, is added successively 100 μL of a solution of tert-butyl piperidine-4-carboxylate (1.9 mg, 10 μmoles, 1 eq) in Dimethylformamide, and di-isopropylamine (6 μL, 40 μmoles, 4 eq). The resulting solution is stirred at room temperature for one hour then transferred into a new container loaded with Cyanoborohydride polymer supported (20 mg, 40 μmoles, 4 eq). The reaction is stirred at room temperature for another 8 h after which the solution is transferred into a 96 well filter plate and the solid filtered off. The residue is rinsed twice with 100 μL of DMF. The combined fractions are dried under reduced pressure to afford a brown oily residue. The crude mixture is treated with 400 μL of a 1/1 vol/vol Trifluoroacetic acid/Dichloromethane mixture for 2H at room temperature. After drying under reduced pressure the deprotected carboxylic acid is used without further purification for the next step.

### 7-cyclopentyl-2-((5-(4-(1-((1-(5-(2,6-dioxopiperidin-3-yl)pyridin-3-yl)piperidin-4-yl)methyl)piperidine-4-carbonyl)piperazin-1-yl)pyridin-2-yl)amino)-N,N-dimethyl-7H-pyrrolo[2,3-d]pyrimidine-6-carboxamide

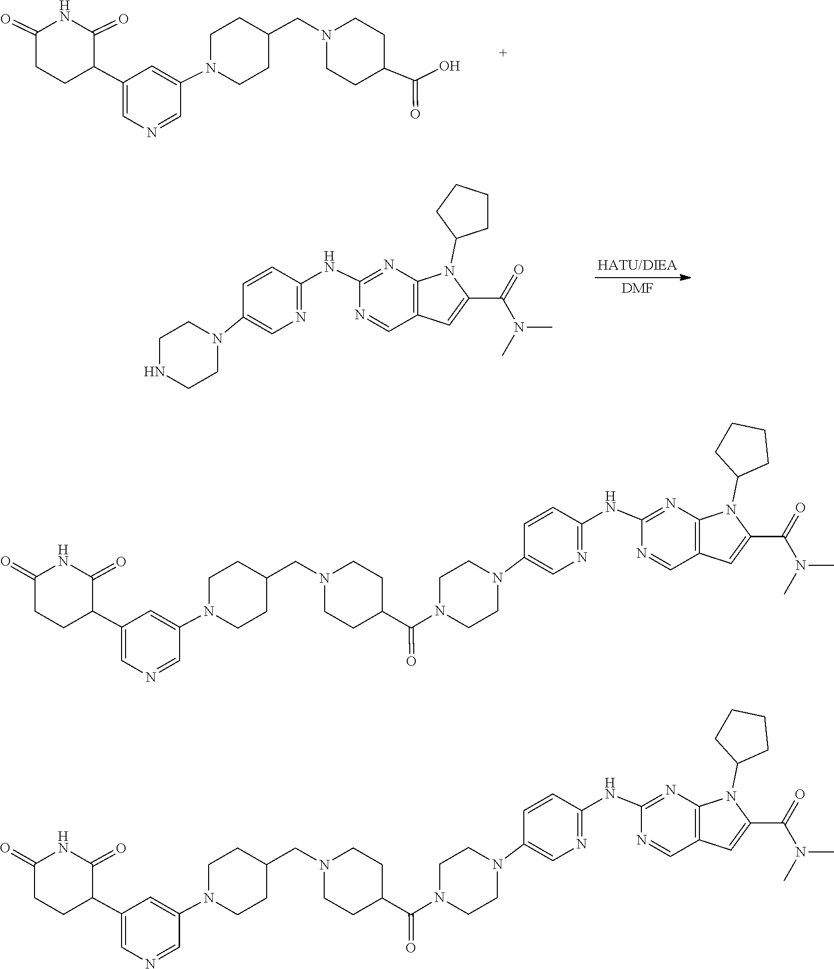

To a solution of 1-((1-(5-(2,6-dioxopiperidin-3-yl)pyridin-3-yl)piperidin-4-yl)methyl)piperidine-4-carboxylic acid (4.1 mg, 10 μmoles) in DMF 200 μL is added successively DIEA (40 umoles, 4 eq) and HATU (100 μL of 100 mM stock solution in DMF, 1 eq) at room temperature, the reaction mixture is stirred for 15 minutes upon then a solution of 7-cyclopentyl-N,N-dimethyl-2-((5-(piperazin-1-yl)pyridin-2-yl)amino)-7H-pyrrolo[2,3-d]pyrimidine-6-carboxamide (4.3 mg in 100 μL DMF, 10 μmoles, 1 eq) is added in one portion. The reaction mixture is stirred for another 8 hours and the crude compound purified by preparative liquid chromatography to yield the title compound (0.6 mg).

### Example 100: R1-R2 Pair Synthesis: R1=Har1; R2=0147

### 3-(5-(4-(piperazin-1-ylmethyl)piperidin-1-yl)pyridin-3-yl)piperidine-2,6-dione

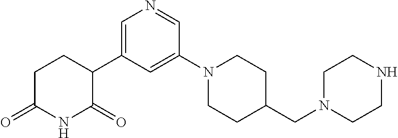 [SMILES: O=C1CCC(c2cncc(N3CCC(CN4CCNCC4)CC3)c2)C(=O)N1]

To a solution of 1-(5-(2,6-dioxopiperidin-3-yl)pyridin-3-yl)piperidine-4-carbaldehyde (3.01 mg, 10 μmoles) in 100 μL of a 1/1 vol/vol Dichloroethane/Dimethylformamide mixture, is added successively 100 μL of a solution of tert-butyl piperazine-1-carboxylate (1.9 mg, 10 μmoles, 1 eq) in Dimethylformamide, and di-isopropylamine (6 μL, 40 μmoles, 4 eq). The resulting solution is stirred at room temperature for one hour then transferred into a new container loaded with Cyanoborohydride polymer supported (20 mg, 40 moles, 4 eq). The reaction is stirred at room temperature for another 8 h after which the solution is transferred into a 96 well filter plate and the solid filtered off. The residue is rinsed twice with 100 μL of DMF. The combined fractions are dried under reduced pressure to afford a brown oily residue. The crude mixture is treated with 400 μL of 1/1 vol mixture of dichloromethane/trifluoro acetic acid for 2 h, then dried under reduced pressure. The brown oil is redissolved in 400 μL of a 1/1 vol dimethylformamide/Toluene mixture before being dried down under reduced pressure. The resulting crude (6 mg, 100%) is used without further preparation.

### Example 101: R3-R4 Pair Synthesis: R3=0147; R4=1106 (AMG-232)

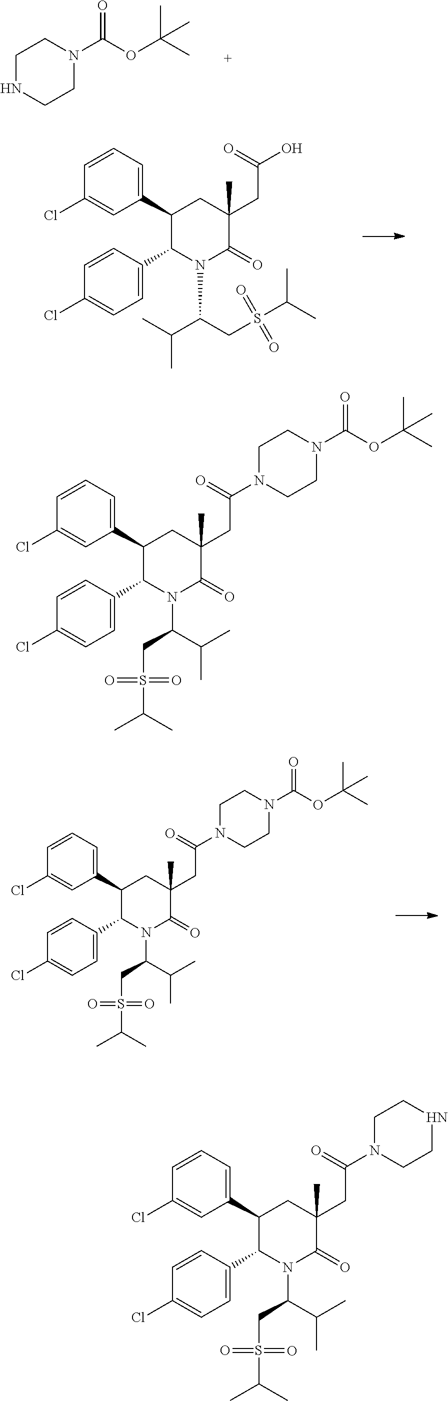 [SMILES: CC(C)(C)OC(=O)N1CCNCC1 ; CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; CC(C)[C@@H](CS(=O)(=O)C(C)C)N1C(=O)[C@@](C)(CC(=O)N2CCN(C(=O)OC(C)(C)C)CC2)C[C@H](c2cccc(Cl)c2)[C@H]1c1ccc(Cl)cc1 ; CC(C)[C@@H](CS(=O)(=O)C(C)C)N1C(=O)[C@@](C)(CC(=O)N2CCN(C(=O)OC(C)(C)C)CC2)C[C@H](c2cccc(Cl)c2)[C@H]1c1ccc(Cl)cc1 ; CC(C)[C@@H](CS(=O)(=O)C(C)C)N1C(=O)[C@@](C)(CC(=O)N2CCNCC2)C[C@H](c2cccc(Cl)c2)[C@H]1c1ccc(Cl)cc1]

To a solution of 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid (5.7 mg, 10 moles) in DMF 100 μL is added successively DIEA (40 moles, 4 eq) and HATU (100 μL of 100 mM stock solution in DMF, 1 eq) at room temperature, the reaction mixture is stirred for 15 minutes upon then a solution of tert-butyl piperazine-1-carboxylate (1.9 mg in 100 μL DMF, 10 μmoles, 1 eq) is added in one portion. The reaction mixture is stirred for another 8 hours then dried down under reduced pressure. The resulting oil is treated with 400 μL of DMC/TFA 1/1 for 2 hours and the resulting mixture is dried down under reduced pressure.

### Example 102: Mass Spectrometry Data

Compounds 351-485 provided the following calculated and observed masses:

| | | | | Calc'd | Obs'd |
|---|---|---|---|---|---|
| Cpd # | Code | Target | Mol Formula | (M + H) | (M + H) |
| 349 | Har1-0172-H06 | CDK4/6 | C₅₅H₇₄Cl₂N₆O₆S | 1017.49 | 1017.29 |
| 350 | Har1-0212-H02 | CDK4/6 | C₄₅H₅₈N₁₂O₄ | 831.49 | 831.19 |
| 351 | Har1-212-H04 | SHP-2 | C₃₈H₄₇Cl₂N₉O₃ | 748.29 | 748.09 |
| 352 | Har1-H17 | BCR-ABL | C₃₆H₄₁ClN₁₀O₃S | 729.29 | 729.09 |
| 353 | Har3-H17 | BCR-ABL | C₃₆H₄₁ClN₁₀O₃S | 729.29 | 729.09 |
| 354 | Har1-H19 | Pan kinase | C₃₈H₄₃ClN₁₀O₃ | 723.29 | 723.19 |
| 355 | Har4-0147-0173-H04 | SHP-2 | C₅₃H₆₅Cl₂N₁₁O₇ | 1038.49 | 1038.39 |
| 356 | Har4-0147-0173-H05 | FGFR1/3 | C₅₃H₆₅Cl₂N₁₁O₇ | 1038.49 | 1038.39 |
| 357 | Har1-0147-0173-H14 | BCR-ABL | C₄₇H₅₄F₃N₉O₆ | 898.39 | 898.39 |
| 358 | Har4-0147-0173-H14 | BCR-ABL | C₄₈H₅₅F₃N₈O₆ | 897.39 | 897.49 |
| 359 | Har1-0212-H05 | FGFR1/3 | C₄₆H₅₅Cl₂N₁₁O₆ | 928.39 | 928.49 |
| 360 | Har2-0212-H05 | FGFR1/3 | C₄₆H₅₅Cl₂N₁₁O₆ | 928.39 | 928.29 |
| 361 | Har1-0212-H14 | BCR-ABL | C₄₁H₄₅F₃N₈O₅ | 787.39 | 787.29 |
| 362 | Har3-0147-0173-H14 | BCR-ABL | C₄₁H₄₅F₃N₈O₅ | 787.39 | 787.19 |
| 363 | Har1-0117-H06 | MDM2 | C₄₉H₆₄Cl₂N₆O₆S | 935.39 | 934.99 |
| 364 | Har1-0225-H18 | IRAK1 | C₄₉H₆₀F₃N₉O₅ | 912.49 | 912.59 |
| 365 | Har2-0117-H06 | MDM2 | C₄₉H₆₄Cl₂N₆O₆S | 935.39 | 935.39 |
| 366 | Har2-0225-H18 | IRAK1 | C₄₉H₆₀F₃N₉O₅ | 912.49 | 912.29 |
| 367 | Har2-0225-H05 | FGFR1/3 | C₄₆H₅₅Cl₂N₁₁O₆ | 928.39 | 928.29 |
| 368 | Har4-0225-H05 | FGFR1/3 | C₄₇H₅₆Cl₂N₁₀O₆ | 927.39 | 927.29 |
| 369 | Har5-0225-H05 | FGFR1/3 | C₄₇H₅₆Cl₂N₁₀O₆ | 927.39 | 927.29 |
| 370 | Har5-0225-H14 | BCR-ABL | C₄₂H₄₆F₃N₇O₅ | 786.39 | 786.29 |
| 371 | Har4-H01 | SHP-2 | C₄₁H₄₉N₉O₄ | 732.39 | 732.39 |
| 372 | Har2-H01 | CDK4/6 | C₄₀H₄₈N₁₀O₄ | 733.39 | 733.29 |
| 373 | Har1-H01 | CDK4/6 | C₄₀H₄₈N₁₀O₄ | 733.39 | 733.29 |
| 374 | Har5-H01 | FGFR1/3 | C₄₁H₄₉N₉O₄ | 732.39 | 732.29 |
| 375 | Har4-H02 | SHP-2 | C₄₀H₅₀N₁₀O₃ | 360.19 | 360.09 |
| 376 | Har2-H02 | CDK4/6 | C₃₉H₄₉N₁₁O₃ | 720.39 | 720.39 |
| 377 | Har1-H02 | CDK4/6 | C₃₉H₄₉N₁₁O₃ | 720.39 | 720.39 |
| 378 | Har5-H02 | CDK4/6 | C₄₀H₅₀N₁₀O₃ | 719.39 | 719.29 |
| 379 | Har4-H03 | SHP-2 | C₃₃H₄₁ClN₈O₂S | 649.29 | 648.99 |
| 380 | Har2-H03 | SHP-2 | C₃₂H₄₀ClN₉O₂S | 650.29 | 650.29 |
| 381 | Har5-H03 | SHP-2 | C₃₃H₄₁ClN₈O₂S | 649.29 | 649.09 |
| 382 | Har2-H04 | SHP-2 | C₃₂H₃₈Cl₂N₈O₂ | 637.29 | 636.89 |
| 383 | Har1-H04 | SHP-2 | C₃₂H₃₈Cl₂N₈O₂ | 637.29 | 636.89 |
| 384 | Har5-H04 | SHP-2 | C₃₃H₃₉Cl₂N₇O₂ | 636.29 | 636.69 |
| 385 | Har4-H05 | FGFR1/3 | C₄₁H₄₇Cl₂N₉O₅ | 816.29 | 816.19 |
| 386 | Har1-H05 | FGFR1/3 | C₄₀H₄₆Cl₂N₁₀O₅ | 817.29 | 817.49 |
| 387 | Har5-H05 | FGFR1/3 | C₄₁H₄₇Cl₂N₉O₅ | 816.29 | 816.19 |
| 388 | Har4-H10 | MEK | C₄₁H₄₁FIN₇O₅ | 858.19 | 857.99 |
| 389 | Har2-H10 | MEK | C₄₀H₄₀FIN₈O₅ | 859.19 | 858.99 |
| 390 | Har1-H10 | MEK | C₄₀H₄₀FIN₈O₅ | 859.19 | 859.09 |
| 391 | Har5-H10 | MEK | C₄₁H₄₁FIN₇O₅ | 858.19 | 858.09 |
| 392 | Har4-H17 | BCR-ABL | C₃₇H₄₂ClN₉O₃S | 728.29 | 728.19 |
| 393 | Har2-H17 | BCR-ABL | C₃₆H₄₁ClN₁₀O₃S | 729.29 | 729.29 |
| 394 | Har1-H17 | BCR-ABL | C₃₆H₄₁ClN₁₀O₃S | 729.29 | 729.19 |
| 395 | Har5-H17 | BCR-ABL | C₃₇H₄₂ClN₉O₃S | 728.29 | 728.19 |
| 396 | Har4-H19 | Pan kinase | C₃₉H₄₄ClN₉O₃ | 722.29 | 722.19 |
| 397 | Har2-H19 | Pan kinase | C₃₈H₄₃ClN₁₀O₃ | 723.29 | 723.19 |
| 398 | Har1-H19 | Pan kinase | C₃₈H₄₃ClN₁₀O₃ | 723.29 | 723.19 |
| 399 | Har5-H19 | Pan kinase | C₃₉H₄₄ClN₉O₃ | 722.29 | 722.29 |
| 400 | Har1-0212-H05 | FGFR1/3 | C₄₆H₅₅Cl₂N₁₁O₆ | 928.39 | 928.29 |
| 401 | Har2-0172-H06 | MDM2 | C₅₅H₇₄Cl₂N₆O₆S | 1017.49 | 1017.69 |
| 402 | Har3-0172-H06 | MDM2 | C₅₅H₇₄Cl₂N₆O₆S | 1017.49 | 1017.59 |
| 403 | Har3-0212-H05 | FGFR1/3 | C₄₆H₅₅Cl₂N₁₁O₆ | 928.39 | 928.49 |
| 404 | Har4-0172-H06 | MDM2 | C₅₅H₇₄Cl₂N₆O₆S | 1017.49 | 1017.39 |
| 405 | Har5-0212-H17 | BCR-ABL | C₄₂H₅₀ClN₁₁O₄S | 840.39 | 840.29 |
| 406 | Har3-0212-H10 | MEK | C₄₆H₄₉FIN₉O₆ | 970.29 | 970.39 |
| 407 | Har5-0212-H10 | MEK | C₄₇H₅₀FIN₈O₆ | 969.29 | 969.09 |
| 408 | Har3-H01 | CDK4/6 | C₄₀H₄₈N₁₀O₄ | 733.39 | 733.29 |
| 409 | Har3-H02 | CDK4/6 | C₃₉H₄₉N₁₁O₃ | 720.39 | 720.39 |
| 410 | Har1-H20 | Pan kinase | C₄₉H₅₁F₂N₇O₇ | 888.39 | 888.39 |
| 411 | Har3-H04 | SHP-2 | C₃₂H₃₈Cl₂N₈O₂ | 637.29 | 637.19 |
| 412 | Har3-H05 | FGFR1/3 | C₄₀H₄₆Cl₂N₁₀O₅ | 817.29 | 817.09 |
| 413 | Har2-H05 | FGFR1/3 | C₄₀H₄₆Cl₂N₁₀O₅ | 817.29 | 817.19 |
| 414 | Har3-H10 | MEK | C₄₀H₄₀FIN₈O₅ | 859.19 | 859.09 |
| 415 | Har4-H20 | Pan kinase | C₅₀H₅₂F₂N₆O₇ | 887.39 | 887.39 |
| 416 | Har4-H10 | MEK | C₄₁H₄₁FIN₇O₅ | 858.19 | 858.19 |
| 417 | Har3-H10 | MEK | C₄₀H₄₀FIN₈O₅ | 859.19 | 859.09 |
| 418 | Har2-H10 | MEK | C₄₀H₄₀FIN₈O₅ | 859.19 | 859.09 |
| 419 | Har1-H10 | MEK | C₄₀H₄₀FIN₈O₅ | 859.19 | 859.09 |
| 420 | Har5-H10 | MEK | C₄₁H₄₁FIN₇O₅ | 858.19 | 858.09 |
| 421 | Har4-0225-H14 | BCR-ABL | C₄₂H₄₆F₃N₇O₅ | 786.39 | 786.19 |
| 422 | Har3-H14 | BCR-ABL | C₃₅H₃₆F₃N₇O₄ | 676.29 | 676.19 |
| 423 | Har4-H16 | MALT1 | C₃₄H₃₆Cl₂N₁₂O₃ | 731.19 | 731.19 |
| 424 | Har3-H16 | MALT1 | C₃₃H₃₅Cl₂N₁₃O₃ | 732.19 | 732.19 |
| 425 | Har2-H16 | MALT1 | C₃₃H₃₅Cl₂N₁₃O₃ | 732.19 | 732.19 |
| 426 | Har1-H16 | MALT1 | C₃₃H₃₅Cl₂N₁₃O₃ | 732.19 | 732.19 |
| 427 | Har5-H16 | MALT1 | C₃₄H₃₆Cl₂N₁₂O₃ | 731.19 | 731.19 |
| 428 | Har3-H17 | BCR-ABL | C₃₆H₄₁ClN₁₀O₃S | 729.29 | 729.29 |
| 429 | Har1-H14 | BCR-ABL | C₃₅H₃₆F₃N₇O₄ | 676.29 | 676.19 |
| 430 | Har5-H14 | BCR-ABL | C₃₆H₃₇F₃N₆O₄ | 675.29 | 675.09 |
| 431 | Har4-H18 | IRAK1 | C₄₄H₅₂F₃N₇O₄ | 800.39 | 800.39 |
| 432 | Har2-H18 | IRAK1 | C₄₃H₅₁F₃N₈O₄ | 801.39 | 801.79 |
| 433 | Har3-H18 | IRAK1 | C₄₃H₅₁F₃N₈O₄ | 801.39 | 801.39 |
| 434 | Har3-H19 | Pan kinase | C₃₈H₄₃ClN₁₀O₃ | 723.29 | 723.19 |
| 435 | Har2-H20 | Pan kinase | C₄₉H₅₁F₂N₇O₇ | 888.39 | 888.29 |
| 436 | Har1-H18 | IRAK1 | C₄₃H₅₁F₃N₈O₄ | 801.39 | 801.29 |
| 437 | Har5-H18 | IRAK1 | C₄₄H₅₂F₃N₇O₄ | 800.39 | 800.59 |
| 438 | Har5-H20 | Pan kinase | C₅₀H₅₂F₂N₆O₇ | 887.39 | 887.29 |
| 439 | Har2-0212-H03 | SHP-2 | C₄₆H₅₅Cl₂N₁₁O₆ | 928.39 | 928.29 |
| 440 | Har2-0212-H05 | FGFR1/3 | C₄₆H₅₅Cl₂N₁₁O₆ | 928.39 | 928.29 |
| 441 | Har2-0212-0117-H08 | COVID | C₅₆H₆₂ClN₉O₇S | 1040.39 | 1040.29 |
| 442 | Har2-0212-H10 | MEK | C₄₆H₄₉FIN₉O₆ | 970.29 | 970.19 |
| 443 | Har1-0172-H11 | MEK | C₄₄H₅₂BrF₂N₉O₅ | 906.29 | 906.29 |
| 444 | Har2-0147-H11 | MEK | C₃₇H₄₀BrF₂N₉O₅ | 810.19 | 810.59 |
| 445 | Har2-0172-H11 | MEK | C₄₄H₅₂BrF₂N₉O₅ | 906.29 | 906.39 |
| 446 | Har4-0147-H11 | MEK | C₃₈H₄₁BrF₂N₈O₅ | 809.19 | 809.09 |
| 447 | Har4-0172-H11 | MEK | C₄₅H₅₃BrF₂N₈O₅ | 905.29 | 905.19 |
| 448 | Har5-0147-H11 | MEK | C₃₈H₄₁BrF₂N₈O₅ | 809.19 | 809.19 |
| 449 | Har1-0212-H12 | MEK | C₄₆H₄₉FIN₉O₆ | 970.29 | 970.19 |
| 450 | Har2-0212-H12 | MEK | C₄₆H₄₉FIN₉O₆ | 970.29 | 970.19 |
| 451 | Har3-0212-H10 | MEK | C₄₆H₄₉FIN₉O₆ | 970.29 | 970.19 |
| 452 | Har4-0212-H10 | MEK | C₄₇H₅₀FIN₈O₆ | 969.29 | 969.19 |
| 453 | Har1-0212-H16 | MALT1 | C₃₉H₄₄Cl₂N₁₄O₄ | 843.29 | 843.79 |
| 454 | Har2-0212-H16 | MALT1 | C₃₉H₄₄Cl₂N₁₄O₄ | 843.29 | 843.29 |
| 455 | Har3-0212-H16 | MALT1 | C₃₉H₄₄Cl₂N₁₄O₄ | 843.29 | 843.29 |
| 456 | Har5-0212-H16 | MALT1 | C₄₀H₄₅Cl₂N₁₃O₄ | 842.29 | 842.19 |
| 457 | Har2-0212-H17 | BCR-ABL | C₄₂H₅₀ClN₁₁O₄S | 840.39 | 840.29 |
| 458 | Har4-0212-H18 | IRAK1 | C₅₀H₆₁F₃N₈O₅ | 911.49 | 911.59 |
| 459 | Har5-0212-H18 | IRAK1 | C₅₀H₆₁F₃N₈O₅ | 911.49 | 911.39 |
| 460 | Har2-0212-H19 | Pan kinase | C₄₄H₅₂ClN₁₁O₄ | 834.39 | 834.29 |
| 461 | Har5-0212-H19 | Pan kinase | C₄₅H₅₃ClN₁₀O₄ | 833.39 | 833.29 |
| 462 | Har2-0212-H20 | Pan kinase | C₅₅H₆₀F₂N₈O₈ | 999.49 | 999.39 |
| 463 | Har1-0117-H08 | COVID | C₅₀H₅₃ClN₈O₆S | 929.39 | 929.39 |
| 464 | Har2-0117-H08 | COVID | C₅₀H₅₃ClN₈O₆S | 929.39 | 929.69 |
| 465 | Har3-0225-H02 | CDK4/6 | C₄₅H₅₈N₁₂O₄ | 831.49 | 831.39 |
| 466 | Har4-0225-H02 | CDK4/6 | C₄₆H₅₉N₁₁O₄ | 830.49 | 830.39 |
| 467 | Har3-0225-H19 | SHP-2 | C₄₄H₅₂ClN₁₁O₄ | 834.39 | 834.29 |
| 468 | Har4-0225-H19 | Pan kinase | C₄₅H₅₃ClN₁₀O₄ | 833.39 | 833.39 |
| 469 | Har1-0225-H02 | CDK4/6 | C₄₅H₅₈N₁₂O₄ | 831.49 | 831.29 |
| 470 | Har3-0117-H08 | COVID | C₅₀H₅₃ClN₈O₆S | 929.39 | 929.29 |
| 471 | Har4-0117-H08 | COVID | C₅₁H₅₄ClN₇O₆S | 928.39 | 928.59 |
| 472 | Har5-0117-H08 | COVID | C₅₁H₅₄ClN₇O₆S | 928.39 | 928.29 |
| 473 | Har3-0225-H10 | MEK | C₄₆H₄₉FIN₉O₆ | 485.59 | 485.69 |
| 474 | Har3-0225-H10 | MEK | C₄₆H₄₉FIN₉O₆ | 970.29 | 970.09 |
| 475 | Har5-0225-H10 | MEK | C₄₇H₅₀FIN₈O₆ | 969.29 | 969.09 |
| 476 | Har1-0225-H17 | BCR-ABL | C₄₂H₅₀ClN₁₁O₄S | 840.39 | 840.29 |
| 477 | Har2-0225-H17 | BCR-ABL | C₄₂H₅₀ClN₁₁O₄S | 840.39 | 840.39 |
| 478 | Har1-0225-H04 | SHP-2 | C₃₈H₄₇Cl₂N₉O₃ | 748.29 | 748.49 |
| 479 | Har2-0225-H04 | SHP-2 | C₃₈H₄₇Cl₂N₉O₃ | 748.30 | 489.90 |
| 480 | Har4-0225-H04 | SHP-2 | C₃₉H₄₈Cl₂N₈O₃ | 747.29 | 747.29 |
| 481 | Har3-0225-H17 | BCR-ABL | C₄₂H₅₀ClN₁₁O₄S | 840.39 | 840.29 |
| 482 | Har4-0225-H17 | BCR-ABL | C₄₃H₅₁ClN₁₀O₄S | 839.39 | 839.29 |
| 483 | Har5-0225-H17 | BCR-ABL | C₄₃H₅₁ClN₁₀O₄S | 839.39 | 839.79 |
| 484 | Har3-0225-H04 | SHP-2 | C₃₈H₄₇Cl₂N₉O₃ | 748.29 | 748.69 |
| 485 | Har5-0225-H04 | SHP-2 | C₃₉H₄₈Cl₂N₈O₃ | 747.29 | 747.29 |

### Biological Example 1—In Vivo Degradation after Oral Administration

### ITK Degradation HiBiT Assay

Compounds provided herein were assayed in vitro with ITK HiBit cell lines. Compound dilution series (11-point, 5 fold dilutions in DMSO, columns 2-12 with replicate in rows A/B, C/D, E/F, G, H at 2000× the final required concentrations were prepared in 96 well plate (Falcon, cat. no. 353077). Column 1, rows A-H were control DMSO. The 2000× solutions ranged from 2 mM to 1.024 nM (final assay concentration range 1 μM to 0.512 μM). The 2000× solutions were added to cells in 10 μL volume, for a final DMSO concentration of 0.5% and final assay compound concentration of 1×. For the cells, C-terminal HiBiT-tagged Molt4 cells (ATCC CRL-1552, monoclonal cell line clone 1C10) were plated at 1×106 cells/mL, 100 μL/well (100×104 cells/well) in complete RPMI (10% FBS, 1% L-glutamine). The cells were incubated with compound 32 and compound 30 for 4 hrs at 32° C./6% CO₂.

Following incubation, 100 μL of complete Nano-Glo HiBiT Lytic Detection Reagent (Nano-Glo HiBiT Lytic Buffer with 1:50 Nano-Glo HiBiT Lytic Substrate and 1:100 LgBiT Protein; Promega cat. no. N₃O₄₀) was added. Cells were further incubated for 10 min at room temperature (RT). Luminescence units (LU) were read on an EnVision plate reader (Perkin Elmer, 0.1 sec per well). Percent ITK remaining per sample was calculated as follows:

$$\% ITK remaining = 100 - [ \frac{Control LU - Sample LU}{Control LU} ] \times 100$$

Using Graphpad Prism, % ITK remaining values were plotted as a function of compound concentration. To determine DC₅₀ and Dₘₐₓ values, resulting curves were fit to the Prism curve-fitting equation “log(inhibitor) vs response−Variable slope (four parameters)” (reported best fit value IC₅₀ used as DC₅₀). ITK was measured with antibody-based MSD (Meso-Scale Discovery) assays shown below.

### ITK Degradation MSD (Meso Scale Discovery) Assay

Compound dilution series (7-point, 5 fold dilutions in DMSO, rows B-H with replicate in Column 1/2, 3/4, 5/6, 7,8, at 2000× the final required concentrations were prepared in 96 well plate (Falcon, cat. no. 353077). Row A, Column 1-8 were control DMSO. The 2000× solutions ranged from 2 mM to 128 nM (final assay concentration range 1 μM to 64M). The 2000× solutions were added to cells in 10 μL volume, for a final DMSO concentration of 0.5% and final assay compound concentration of 1×. For the cells, either human Jurkat (Clone E6-1 ATCC TIB-152) or Motl4 (ATCC CRL-1552), were plated at 1×106 cells/mL, 100 μL/well (100×104 cells/well) in complete RPMI (10% FBS, 1% L-glutamine). Cells were incubated for 4 or 6 hrs at 32° C./6% CO₂.

Following incubation, plates were centrifuged at 1200 rpm for 5 min. supernatant was removed and 50 μL of cell lysis buffer ((MSD Tris lysis buffer (R60TX), cOmplete Mini EDTA-free protease inhibitor (Sigma 11836170001), Protease Inhibitor Cocktail (Sigma, P2714), Phosphatase Inhibitor Cocktail 2 and 3 (Sigma, P5726 and P0044), Benzonase (Sigma, E1014)) was added to each well. Plate was sealed and shook at 4 C for 30 min. Plate is centrifuged and 45 is removed for plate assay.

Assay plate preparation: Meso Scale Discovery (MSD) multi-array sm spot 96 well plates (Goat anti-Rabbit L45-RA), were blocked with 3% BSA blocking buffer (3% Bovine Serum Albumin (Sigma A3059)+TBS 0.2% Tween-20) for 1 hr with gentle rocking at room temperature. Plate was then washed with 200 μL of 1×TBST (TBS 0.2% Tween-20) 3 times. After last wash, all liquid is removed and 50 μL per well of capture antibody (abcam ITK Y402-ab32507) was added to plate at a 1:1000 dilution in blocking buffer (see above). Plate was sealed and rocked at room temperature for 2 hrs. Plate is then washed 3 times with 200 μL of 1×TBST. After last wash, all liquid is removed and 45 μL of cell lysate (from above) are added to plate. Plate is sealed and rocked overnight at 4 C. Next day, cell lysates are removed, and assay plate is washed 3 times with 200 μL of 1×TBST. After last wash, liquid is removed and detection antibody (CST ITK (2F12) #2380) is added at 50 μL per well at a dilution of 1:1000 in blocking buffer. Plate is sealed and rocked at room temperature for 2 hrs. Plate is washed 3 times with 200 μL of 1×TBST. After last wash, all liquid is removed, MSD Mouse anti-Rabbit sulfo tag (R32AC-1) is diluted to 1:1000 in blocking buffer. 50 μL is added to each well on the plate. Plate is sealed and incubated at room temperature for 1 hr. Plate is washed 3 times with 200 μL of 1×TBST. After last wash, all liquid is removed and 150 μL of 1×MSD Read Buffer T (R92PC) is then added to each well for ECL read out.

For reading ECL signal, plate is read on a Meso Scale Discovery (MSD) MESO Sector S 600 plate reader. Percent ITK in then calculated as described below.

Percent ITK remaining per sample was calculated as follows:

$$\% ITK remaining = 100 - [ \frac{Control ECL - Sample ECL}{Control ECL} ] \times 100$$

Using Graphpad Prism, % ITK remaining values were plotted as a function of compound concentration. To determine DC₅₀ and Dₘₐₓ values, resulting curves were fit to the Prism curve-fitting equation “log(inhibitor) vs response−Variable slope (four parameters)” (reported best fit value IC₅₀ used as DC₅₀).

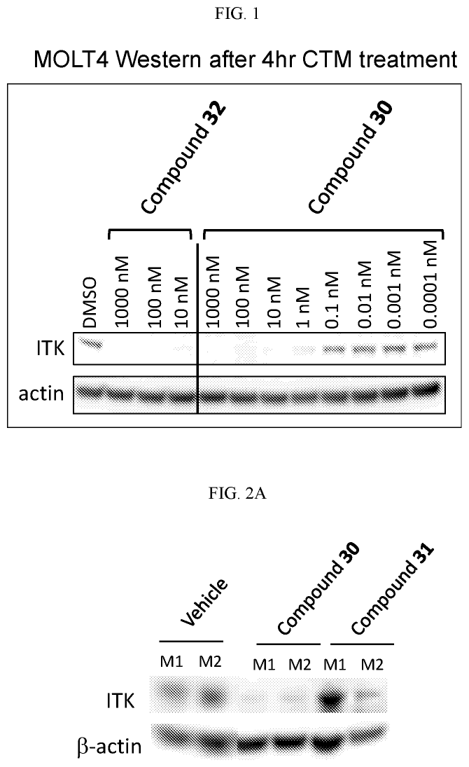

As shown in FIG. 1, compounds 32 and 30 degraded ITK with DC₅₀ values less than 10 nM. The Dmax values were 90-100% each.

### Biological Example 2—In Vivo Degradation after Oral Administration

### Western Assay for ITK Degradation in Mouse Splenocytes

Compounds 30 and 31 were administered to mice orally. After 6 hours, splenocytes cells were harvested. ITK was evaluated by Western blotting. Media was removed and cell pellets were lysed in 100 μL lysis buffer (RIPA buffer (Fisher, PI89901), cOmplete Mini EDTA-free protease inhibitor (Sigma 11836170001), Protease Inhibitor Cocktail (Sigma, P2714), Phosphatase Inhibitor Cocktail 2 and 3 (Sigma, P5726 and P0044), Benzonase (Sigma, E1014)). Cells were lysed overnight at −20° C. Following thaw, cells were centrifuged for 10 min at 13000 rpm, then transferred to new tube. Protein levels were determined by BCA Assay performed according to manufacturer's protocol (EMD Millipore, cat. no. 71285-3). Samples were combined with (4×) LDS Sample Buffer and (10×) Reducing Agent and H₂O to equally load 20 μg protein per lane of a 26-well NuPAGE 4-12% Bis-Tris protein gel (1.0 mm, Thermo cat. no. NP0326). Samples were separated by running gels at constant 150 V in NuPAGE MES SDS Running Buffer. Following electrophoresis, proteins were transferred to nitrocellulose membranes using an iBlot Gel Transfer Device and iBlot Gel Transfer Stacks (Thermo cat. no. IB21001 and IB301001) and transfer method P3 (20V for 7 min). Membranes were blocked for 1 hr in 5% milk solution (TBS (0.2% Tween-20)). Following blocking, membranes were incubated with primary antibody (1:1000 CST ITK (2F12) #2380) overnight at 4° C. with gentle shaking. Blots were washed 2× in TBS (0.2% Tween-20), 30 min per wash. Following washes, blots were incubated in secondary HRP-conjugated antibody (Promega anti-Mouse IgG (H+L) HRP cat. no. W4021), 1:5000 in 5% milk solution (TBS (0.2% Tween-20)), for 1 hr at room temperature with gentle shaking. Blots were washed 2× in TBS (0.2% Tween-20), 30 min per wash. Blots were incubated with 1:1 mix of ECL reagents 1 & 2 (Amersham ECL Western Blotting Detection Reagent, cat. no. RPN2106) for 2 min at room temperature. Bands were visualized using a Protein Simple imager. Blots were then re-probed with a combination of anti-actin antibody (Sigma Monoclonal Mouse Anti-β-Actin (clone AC-15), cat. no. A5441) and secondary HRP-conjugated antibody (Promega anti-Mouse IgG (H+L) HRP, cat. no. W4021) and similar steps were taken for incubation, wash, detection and visualization steps as above. The data was analyzed using Alpha View software. The densitometric reading for each sample band was normalized to that of the corresponding actin band per lane. Approximate % ITK remaining per sample was calculated as follows:

$$\% ITK normalized to actin = [ \frac{ITK band - blot background}{actin band - blot background} ] \\ \% ITK remaining = [ \frac{Normalized ITK Sample}{Mean normalized ITK Control} ] \times 100$$

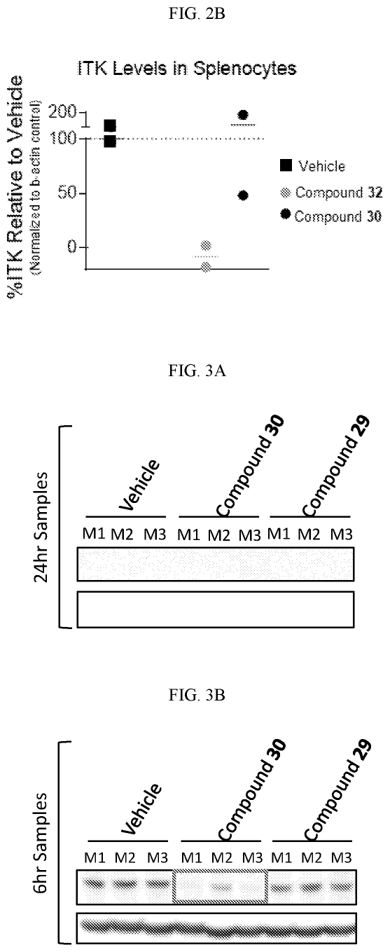

Once % ITK remaining has been calculated for each sample, groups are averaged together to show an means % ITK normalized to actin and relative to control. As shown in FIG. 2A, at least compounds 30 and 31 degraded ITK in vivo at 6 hours and at 24 hours. As shown in FIG. 2B, the percent of ITK remaining decreased substantially compared to vehicle.

### Biological Example 3—In Vivo Degradation after Oral Administration

### Mouse PK Analysis

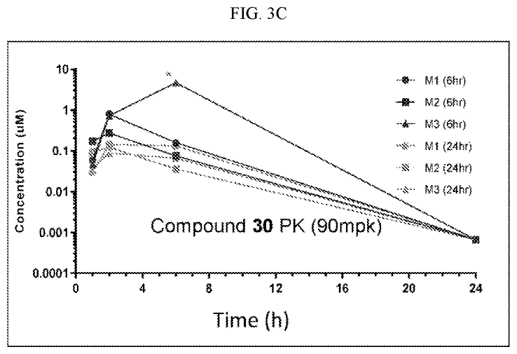

Compounds 30 and 29 were administered to mice orally. After 6 hours or 24 hours, splenocytes cells were harvested. ITK was evaluated by Western blotting. Plasma concentrations were determined by LC/MS/MS. Plasma samples were protein precipitated by addition of 100 μl of acetonitrile containing 50 ng/ml of internal standard. The resulting mixture was vortexed and centrifuged at 4000 RPM for 5 minutes. An aliquot of the resultant supernatant (75 μl) was added to 75 μl of 0.1% formic acid in water to constitute the final sample for injection. Samples were injected on a Shimadzu Exion LC Binary Gradient AD Pump HLPC system connected to a Sciex QTRAP 6500+ mass spectrometer. Five μl of sample was injected onto a Waters Acquity UPLC BEH C18 column 130A (2.1×30 mm, 1.7m) at 40° C. utilizing a flow rate of 700 μl/minute. Mobile Phase A was 0.1% formic acid in water and mobile phase B was 0.1% formic acid in acetonitrile. A linear gradient of 15-95% B over 1.0 minute was used. The mass spectrometer was operated in positive ion electrospray mode with multiple reaction monitoring for maximum sensitivity. Sciex Analyst software (version 1.6.3) was used for LC/MS/MS instrument control and acquisition. Compound concentration was determined against a standard curve of internal standard versus compound peak area ratio. Noncompartmental PK parameters were determined using Phoenix 32 software (version 8.2.0.4383) from Certara. Plasma concentrations from the LC/MS/MS analyses, dose, route of administration, and desired units were utilized for PK parameter calculations. As shown in FIG. 3A, compounds 30 and 29 degraded ITK in vivo at 6 hours, and compounds 30 and 29 degraded ITK at 24 hours. As shown in FIG. 3B, compound 30 was detected in plasma through 6 and 24 hours, respectively. FIG. 3C provides the concentration over time of compound 30 following in vivo oral administration in the 6 hour and 24 hour assays.

### Noncompartmental PK Parameters

Cmax, Tmax and AUClast were all calculated using a WinNonLin Phoenix 64 v 8.2.0.4383. Using non-compartmental analysis, Tmax, Cmax and AUClast were determined as follows:

Tmax—Time of maximum observed concentration.
Cmax—Maximum observed concentration, occurring at time Tmax, as defined above.
AUClast—Area under the curve from the time of dosing to the time of the last measurable (positive) concentration (Tlast).

Results of biological assays are reported in Table 3A-3C

TABLE 3A

| Compound | HiBiT | HiBiT |
|---|---|---|
| No. | DC₅₀ (nM) | D_{Max} (%) |
| 4 | 0.01 | 82 |
| 5 | 10.5 | 85 |
| 6 | 1.6 | 85 |
| 12 | 0.25 | 85 |
| 13 | 1.3 | 91 |
| 14 | 1.8 | 78 |
| 16 | 0.01 | 90 |
| 17 | <0.01 | 89 |
| 18 | 0.68 | 87 |
| 19 | 11 | 73 |
| 20 | <0.001 | 83 |
| 21 | <0.01 | 83 |
| 22 | 0.21 | 90 |
| 23 | 3.9 | 85 |
| 24 | 0.6 | 92 |
| 25 | NC | 48 |
| 26 | 4.6 | 87 |
| 28 | 0.27 | 91 |
| 29 | 0.004 | 76 |
| 30 | <0.001 | 85 |
| 31 | 2 | 84 |
| 32 | 0.48 | 89 |
| 33 | 0.04 | 80 |
| 34 | 2.6 | 60 |
| 35 | <0.01 | 86 |
| 37 | 2.6 | 79 |
| 38 | 1.5 | 74 |
| 39 | 0.09 | 82 |
| 40 | 15 | 68 |
| 41 | — | 27 |
| 42 | 0.003 | 88 |

TABLE 3B

| Compound | | | AUC Last |
|---|---|---|---|
| No. | C_{Max} (μM) | T_{Max} (h) | (h*μM) |
| 4 | 2.43 | 1 | 7.07 |
| 24 | 0.61 | 6 | 1.43 |
| 26 | 7.11 | 6 | 36.5 |
| 27 | 0.609 | 1 | 1.69 |
| 28 | 0.379 | 6 | 1.56 |
| 29 | 0.694 | 3.3 | 1.98 |
| 30 | 0.87 | 2 | 1.25 |
| 31 | 3.08 | 2 | 16.5 |
| 32 | 1.62 | 6 | 22.7 |
| 35 | 2.0 | 2 | 12 |
| 37 | 5.4 | 2 | 72 |
| 38 | 11 | 3.3 | 150 |
| 39 | 0.30 | 6 | 3.9 |
| 42 | 5.1 | 2 | 28 |

TABLE 3C

| | ITK (%) | ITK (%) | ITK (%) |
|---|---|---|---|
| Compound | 90 mpk | 90 mpk | 60 mpk |
| No. | PO, 6 h | PO, 24 h | PO, 24 h |
| 4 | 76 | — | — |
| 6 | 92 | — | — |
| 22 | 113 | — | — |
| 23 | 129 | — | — |
| 24 | 45 | — | — |
| 26 | 11 | — | — |
| 28 | 8 | — | — |
| 29 | 14 | — | — |
| 30 | 20 | — | — |
| 31 | 116 | — | — |
| 32 | 11 | — | — |
| 35 | — | — | >100 |
| 37 | — | — | >100 |
| 38 | 14 | 14 | — |
| 39 | — | — | 93 |

### OTHER EMBODIMENTS

It is to be understood that the foregoing description is intended to illustrate and not limit the scope of this disclosure, which is defined by the scope of the appended claims. Other aspects, advantages, and modifications are within the scope of the following claims.

1. A compound of Formula (I) 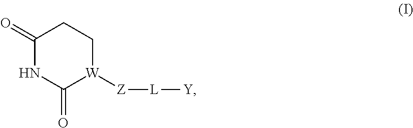
or a stereoisomer and/or a pharmaceutically acceptable salt thereof,
wherein:
W is CH;
Z is selected from the group consisting of: 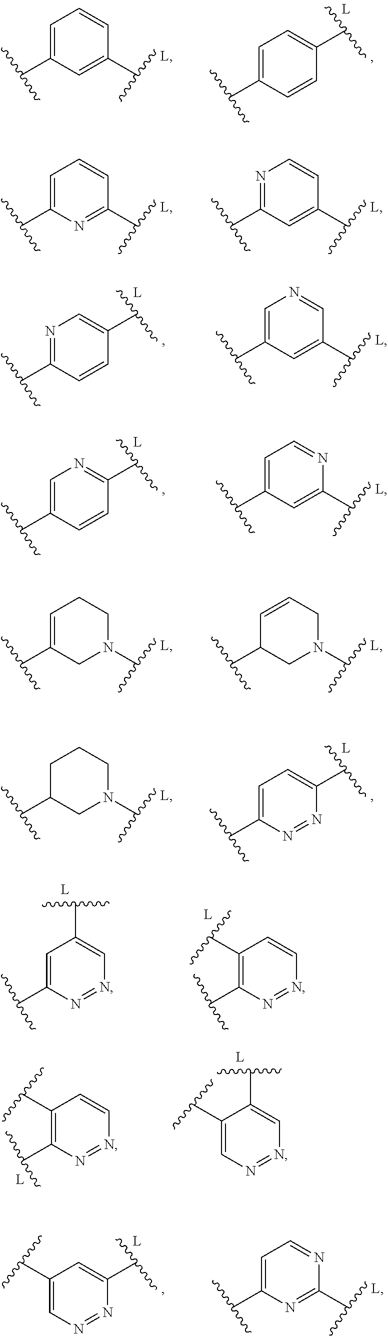 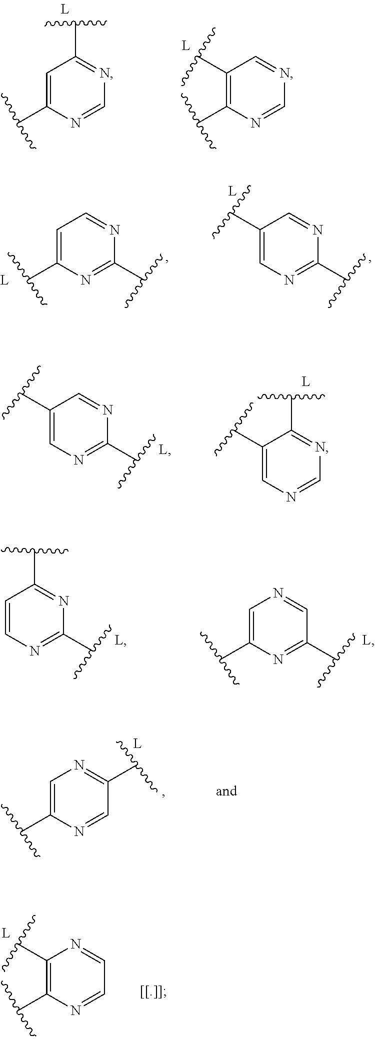
L is a linker group; and
Y is a target binding moiety, which binds to a target protein which is to be degraded by the compound.

2. (canceled)

3. The compound of claim 1, wherein the linker group comprises 3 to 40 carbon atoms, comprising one or more acyclic and/or cyclic moieties, wherein one or more carbons atoms can be replaced optionally by a heteroatom selected from O, N, S, and P, or wherein the linker group comprises 1, 2, 3, 4, 5, 6, 7, 8, 9, or 10 ethylene glycol units; wherein the linker group is optionally substituted.

4. The compound of claim 1, wherein L, the linker group is according to -L¹-L²-L³-L⁴-L⁵-L⁶-L⁷-,
wherein:
-L¹- is absent, —N(R²¹)—, C(R²²)—, C₁₋₈ alkylene, C₂₋₈ alkynylene, Q¹, or Q²;
each -L²-, -L³-, -L⁴-, and -L⁵- is independently, absent, —N(R²¹)—, C(R²²)—, —C(O)—, —O—, —(CH₂—CH₂—O)₁₋₈—, C₁₋₈ alkylene, C₂₋₈ alkynylene, Q¹, Q², or Q³;
each -L⁶- and -L⁷-, is independently, absent, —N(R²¹)—, —C(R²²)—, —C(O)—, —C(O)—N(R²¹)—, —N(R²¹)—C(O), or —C(R²²)—C(O)—N(R²¹);
each Q¹ is a three- to seven-membered heterocycloalkylene comprising at least one nitrogen;
each Q² is a five- to thirteen-membered spiro bicyclic heterocycloalkylene comprising at least one nitrogen;
each Q³ is a three- to six-membered cycloalkylene;
R²¹ is H, or methyl; and
R²² is H, methyl, aryl, or heteroaryl.

5. The compound of claim 1, wherein:
Y, the target binding moiety has the capability to bind to a target protein selected from the group consisting Bruton's tyrosine kinase (BTK); tyrosine-protein kinase (ITK/TSK); bromodomain-containing protein 4 (BRD4); FMS-like tyrosine kinase 3 (FLT-3); Brg/Brahma-associated factors (BAF complex); induced myeloid leukemia cell differentiation protein (MCL-1); signal transducer and activator of transcription 3 (STAT3); barrier-to-autointegration factor (BAF); and BCR-ABL to bring it into close proximity to E3 ligase to effect the protein's ubiquitination and degradation.

6. The compound of claim 1, wherein Y is arylene, heteroaryl, heteroaralkyl, heteroarylene, heterocycle, heterocycloalkyl, or heterocycloalkylaryl, wherein arylene, heteroaryl, heteroaralkyl, heteroarylene, heterocycle, heterocycloalkyl, or heterocycloalkylaryl, each optionally substituted.

7. The compound of claim 1, wherein Y is selected from the group consisting of: 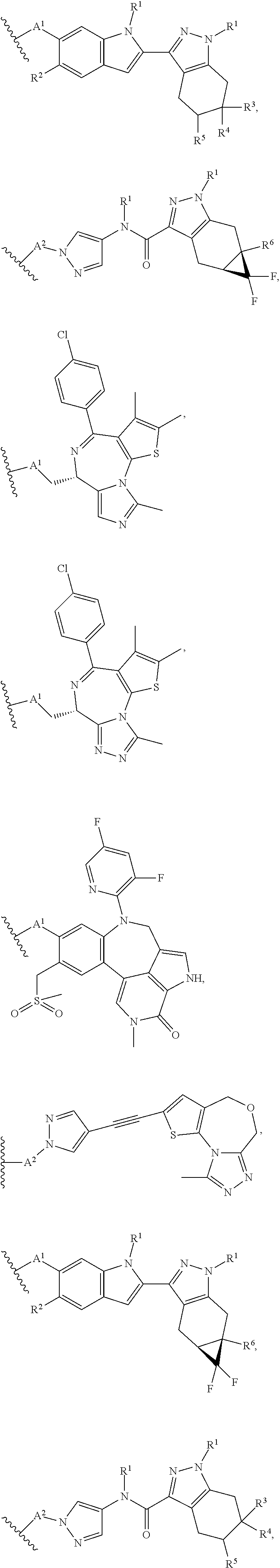 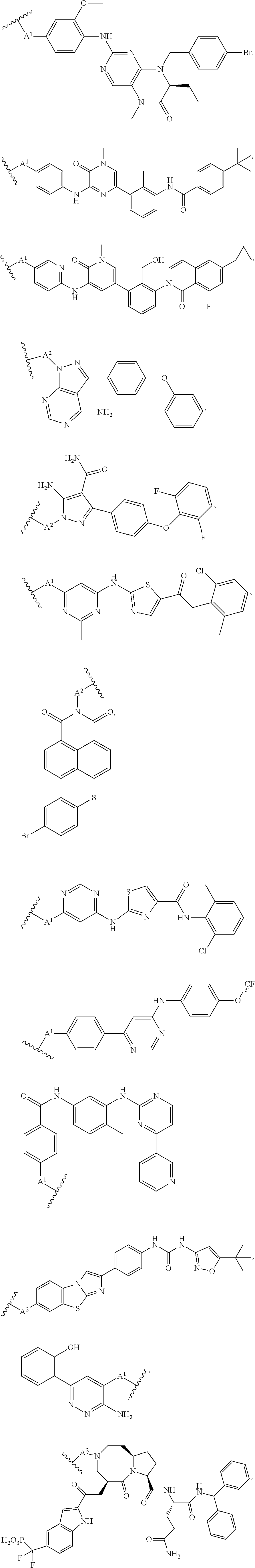 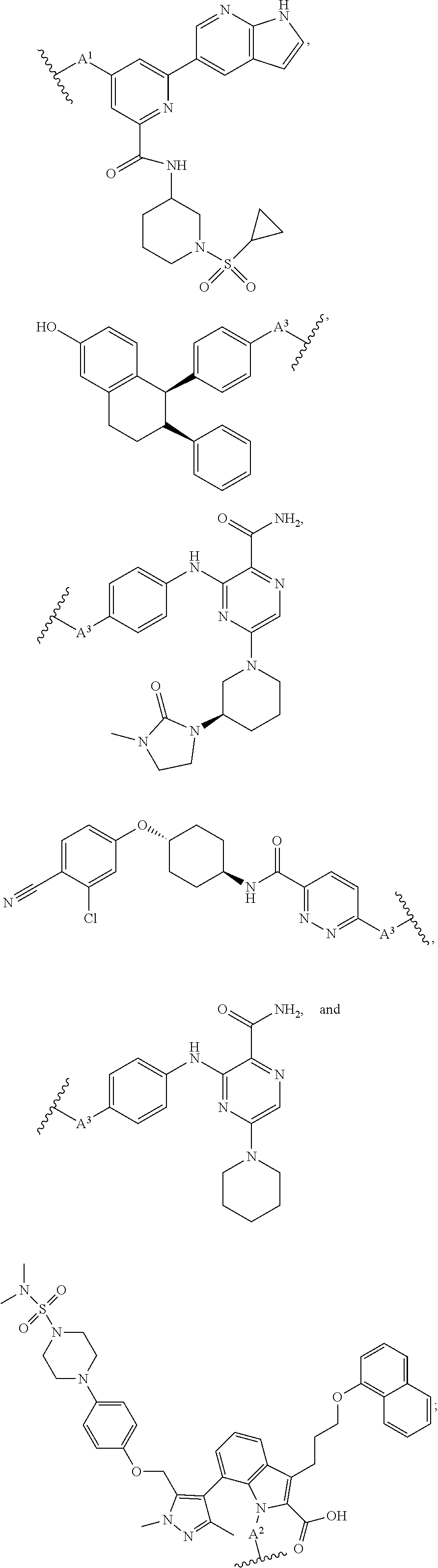
wherein:
R¹ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, or —C(O)heteroaryl;
R² is hydrogen, or halogen;
A¹ is absent, or —O—, —C(O)—, —C(O)—NH—, —C(O)—NH-A^{2a}-, —CH(A^{2a})-, —N(alkyl)-, -alkyl-, or —C(O)—NH-phenol-;
A² is absent, or —C(H)(A^{2a})-, wherein A^{2a} is hydrogen, alkyl, aryl, heterocycle, or heteroaryl;
A³ is a bond;
R³ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, or —C(O)heteroaryl;
R⁴ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, —C(O)heteroaryl, or an alkylene bound to R⁵ to form a substituted cycloalkyl;
R⁵ is hydrogen, or an alkylene bound to R⁴ to form the substituted cycloalkyl;
wherein when R⁵ and R⁴ form the substituted cycloalkyl, then the cycloalkyl is substituted with one or more substituents selected from the group consisting of: alkyl,
alkenyl, alkynyl, cycloalkyl, heterocycloalkyl, aryl, alkaryl, aralkyl, haloalkyl, heteroaryl, cyano, —C(O)alkyl, —C(O)aryl, —C(O)heteroaryl, and halogen; and
R⁶ is hydrogen, or alkyl.

8.-30. (canceled)

31. The compound of claim 4, wherein L comprises:
at least one -Q¹- according to 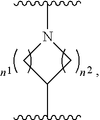
wherein n¹ is one, or two, and n² is one, or two; or
at least one -Q¹- selected from the group consisting of

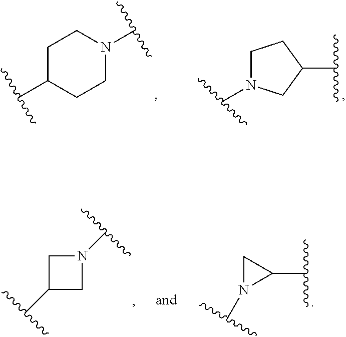 [SMILES: *C1CCN(*)CC1 ; *C1CCN(*)C1 ; *C1CN(*)C1 ; *C1CN1*]

32. (canceled)

33. The compound of claim 4, wherein L comprises:
at least one -Q²- according to 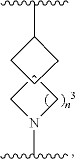
wherein n³ is one, or two;
at least one -Q²- according to 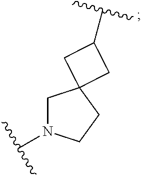 [SMILES: *C1CC2(CCN(*)C2)C1]
at least one -Q²- according to 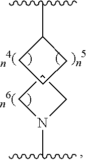
wherein n⁴ is one, or two, n⁵ is one, or two, and n⁶ is one, or two;
at least one -Q²- according to 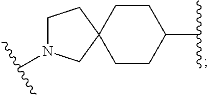 [SMILES: *C1CCC2(CC1)CCN(*)C2]
at least one -Q²- according to 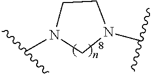
wherein n⁸ is one, or two;
at least one -Q²- according to 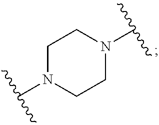 [SMILES: *N1CCN(*)CC1]
at least one -Q²- according to 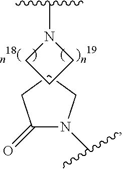
wherein n¹⁸, and n¹⁹ is two, or piperidinyl; or when n¹⁸ is two, then n¹⁹ is three, or azepanyl; or when n¹⁸ is three, then n¹⁹ is two, or azepanyl;
at least one -Q²- according to 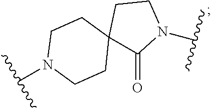 [SMILES: *N1CCC2(CC1)CCN(*)C2=O]
at least one -Q²- according to 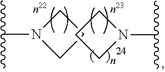
wherein n²² is zero to two; n²³ is zero to two, and n²⁴ is one, or two;
at least one -Q²- according to 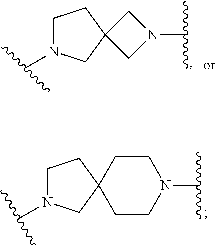 [SMILES: *N1CCC2(C1)CN(*)C2 ; *N1CCC2(CC1)CCN(*)C2]
or
at least one -Q²- according to

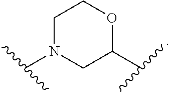 [SMILES: *C1CN(*)CCO1]

34.-44. (canceled)

45. The compound of claim 4, wherein L comprises:
at least one -Q³- according to 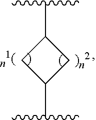
wherein n¹ is one, or two, and n² is one, or two; or
at least one -Q³- selected from the group consisting of

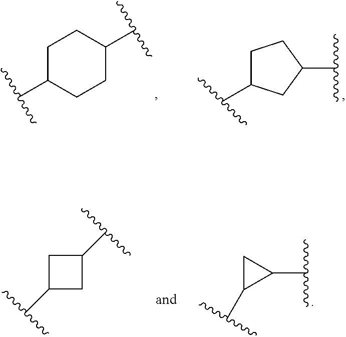 [SMILES: *C1CCC(*)CC1 ; *C1CCC(*)C1 ; *C1CC(*)C1 ; *C1CC1*]

46.-47. (canceled)

48. The compound of claim 1, wherein L, the linker group is selected from: 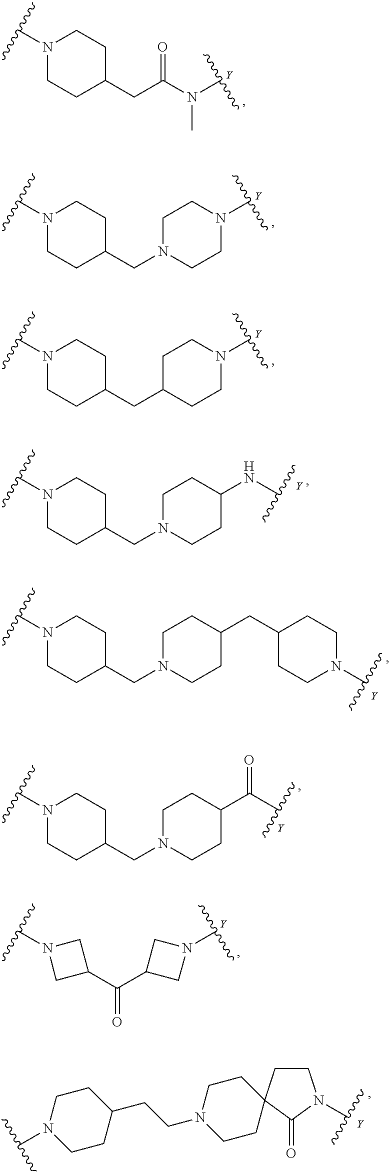 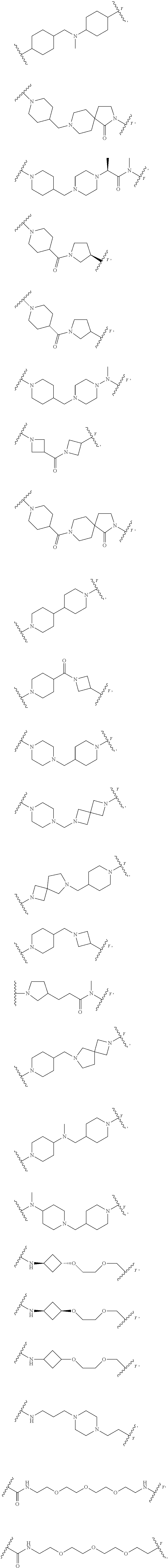 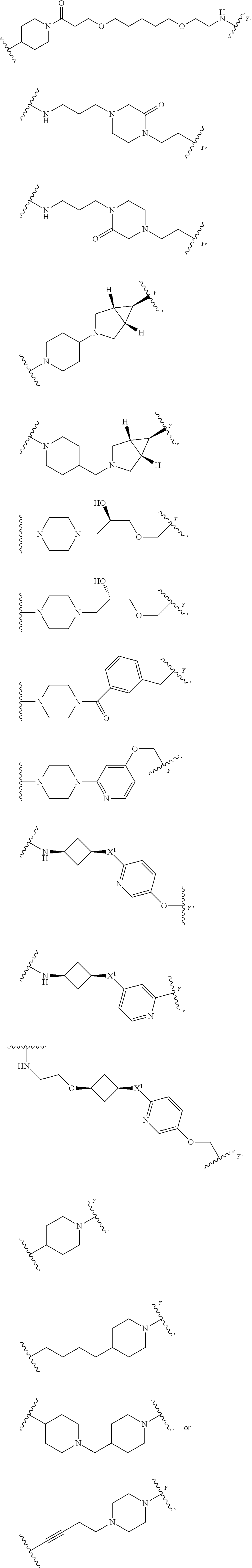
wherein, [symbol] designates attachment to Y.

49. The compound of claim 1, selected from one of the following:
Formula (II): 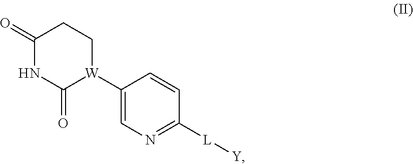
or a stereoisomer and/or a pharmaceutically acceptable salt thereof;
Formula (III): 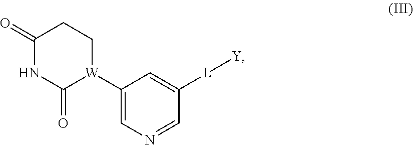
or a stereoisomer and/or a pharmaceutically acceptable salt thereof;
Formula (IV): 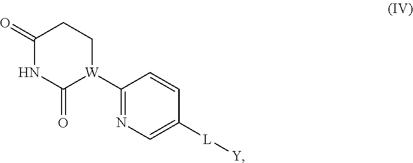
or a stereoisomer and/or a pharmaceutically acceptable salt thereof;
Formula (V): 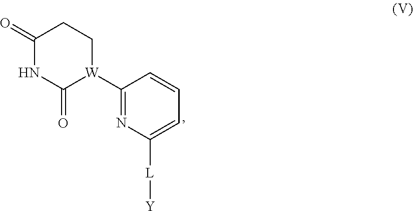
or a stereoisomer and/or a pharmaceutically acceptable salt thereof;
Formula (VI): 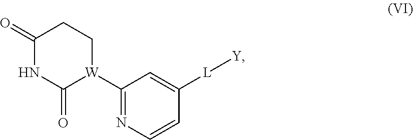
or a stereoisomer and/or a pharmaceutically acceptable salt thereof;
Formula (VII): 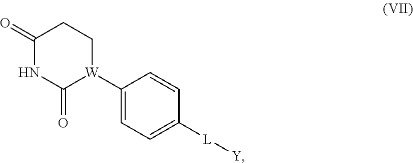
or a stereoisomer and/or a pharmaceutically acceptable salt thereof;
Formula (VIII): 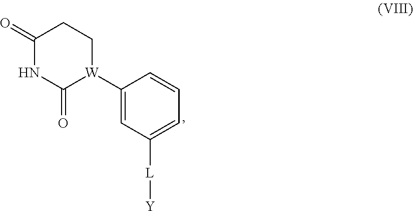
or a stereoisomer and/or a pharmaceutically acceptable salt thereof;
Formula (IX): 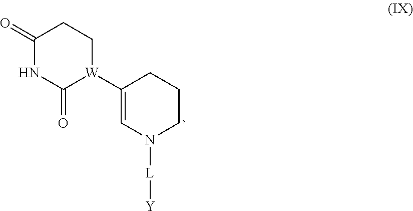
or a stereoisomer and/or a pharmaceutically acceptable salt thereof;
Formula (X): 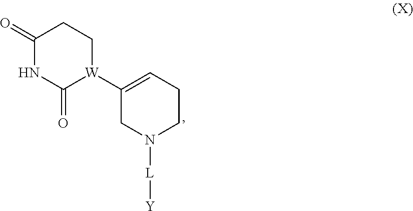
or a stereoisomer and/or a pharmaceutically acceptable salt thereof;
Formula (XI): 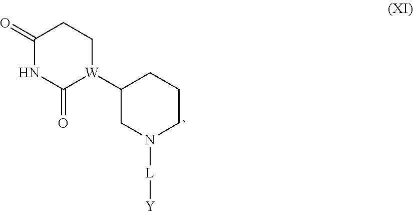
or a stereoisomer and/or a pharmaceutically acceptable salt thereof;
Formula (XII): 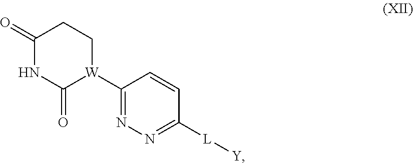
or a stereoisomer and/or a pharmaceutically acceptable salt thereof;
Formula (XIII): 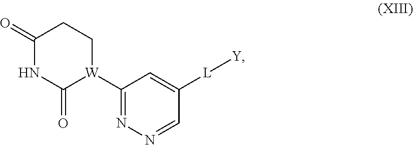
or a stereoisomer and/or a pharmaceutically acceptable salt thereof;
Formula (XIV): 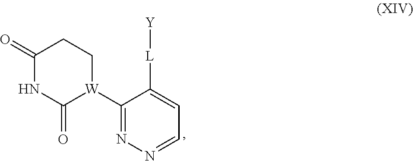
or a stereoisomer and/or a pharmaceutically acceptable salt thereof;
Formula (XV): 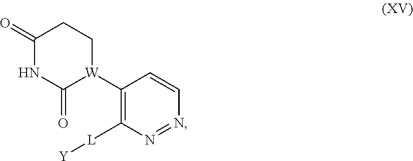
or a stereoisomer and/or a pharmaceutically acceptable salt thereof;
Formula (XVI): 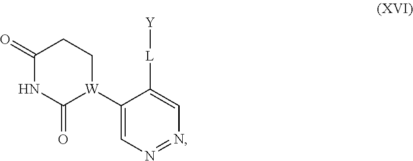
or a stereoisomer and/or a pharmaceutically acceptable salt thereof;
Formula (XVII): 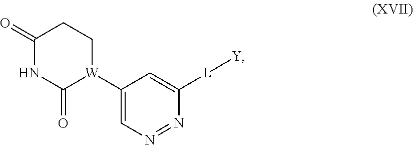
or a stereoisomer and/or a pharmaceutically acceptable salt thereof;
Formula (XVIII): 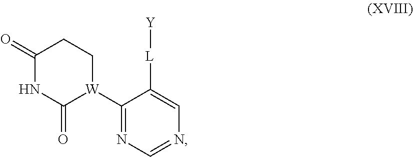
or a stereoisomer and/or a pharmaceutically acceptable salt thereof;
Formula (XXIX): 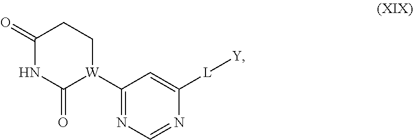
or a stereoisomer and/or a pharmaceutically acceptable salt thereof;
Formula (XX): 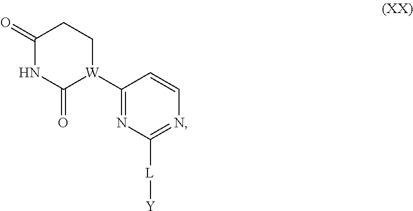
or a stereoisomer and/or a pharmaceutically acceptable salt thereof;
Formula (XXI): 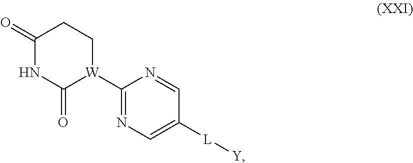
or a stereoisomer and/or a pharmaceutically acceptable salt thereof;
Formula (XXII): 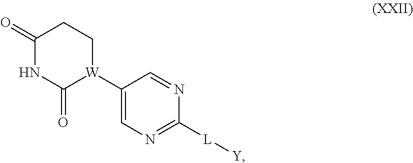
or a stereoisomer and/or a pharmaceutically acceptable salt thereof;
Formula (XXIII): 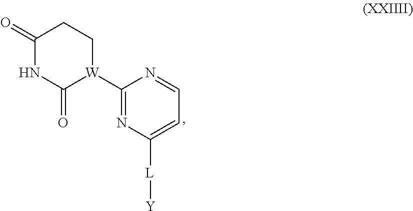
or a stereoisomer and/or a pharmaceutically acceptable salt thereof;
Formula (XXIV): 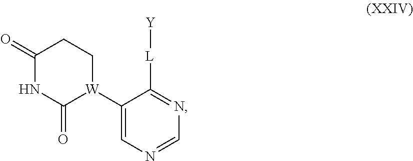
or a stereoisomer and/or a pharmaceutically acceptable salt thereof;
Formula (XXV): 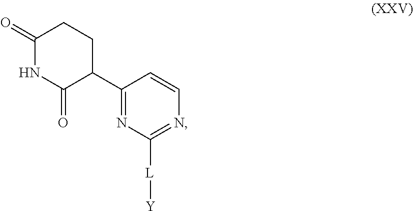
or a stereoisomer and/or a pharmaceutically acceptable salt thereof;
Formula (XXVI): 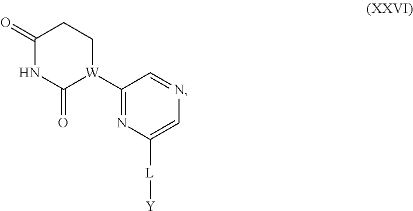
or a stereoisomer and/or a pharmaceutically acceptable salt thereof;
Formula (XXVII): 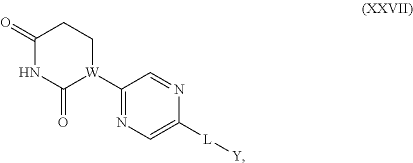
or a stereoisomer and/or a pharmaceutically acceptable salt thereof;
Formula (XXVIII): 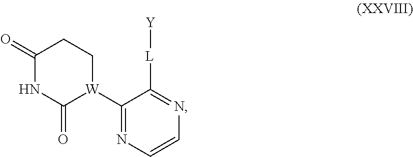
or a stereoisomer and/or a pharmaceutically acceptable salt thereof; and
Formula (XXIX): 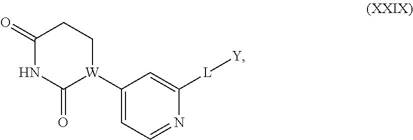
or a stereoisomer and/or a pharmaceutically acceptable salt thereof.

50.-76. (canceled)

77. The compound of claim 1, selected from one of the following:
Formula (Ia): 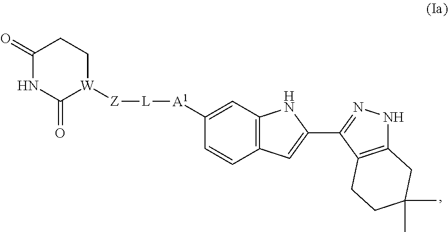
or a stereoisomer and/or a pharmaceutically acceptable salt thereof;
Formula (Ib): 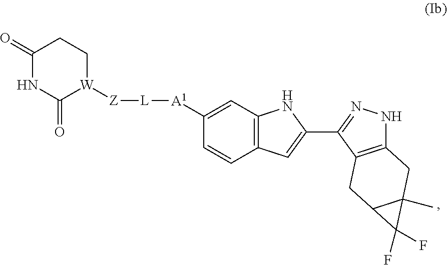
or a stereoisomer and/or a pharmaceutically acceptable salt thereof;
Formula (Ib1): 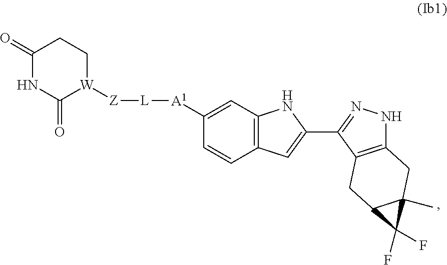
or a stereoisomer and/or a pharmaceutically acceptable salt thereof;
Formula (Ic): 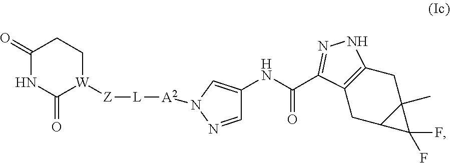
or a stereoisomer and/or a pharmaceutically acceptable salt thereof;
Formula (Id): 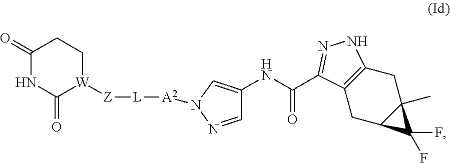
or a stereoisomer and/or a pharmaceutically acceptable salt thereof;
Formula (Ie): 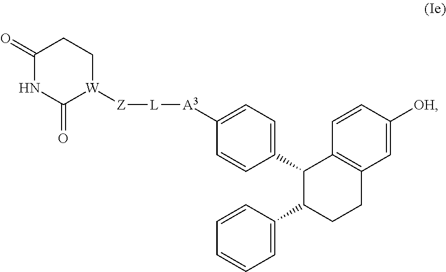
or a stereoisomer and/or a pharmaceutically acceptable salt thereof;
Formula (If): 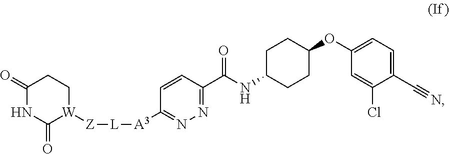
or a stereoisomer and/or a pharmaceutically acceptable salt thereof;
Formula (I1): 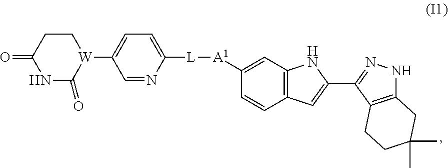
or a stereoisomer and/or a pharmaceutically acceptable salt thereof;
Formula (I2): 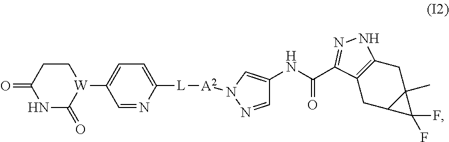
or a stereoisomer and/or a pharmaceutically acceptable salt thereof;
Formula (I2A): 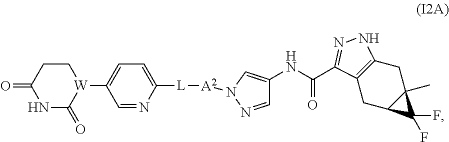
or a stereoisomer and/or a pharmaceutically acceptable salt thereof;
Formula (I3): 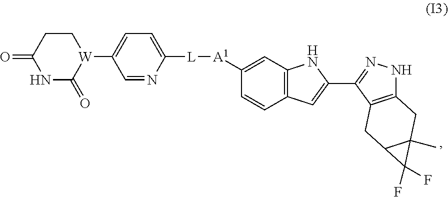
or a stereoisomer and/or a pharmaceutically acceptable salt thereof;
Formula (I3A): 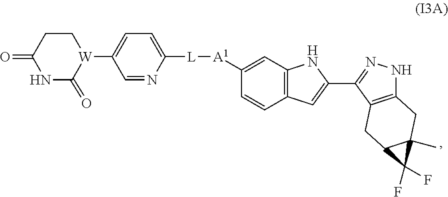
or a stereoisomer and/or a pharmaceutically acceptable salt thereof;
Formula (I4): 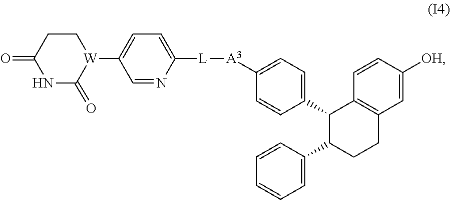
or a stereoisomer and/or a pharmaceutically acceptable salt thereof;
Formula (I5): 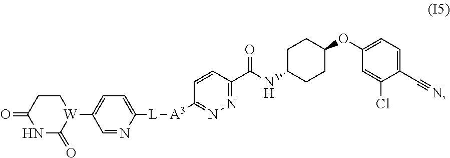
or a stereoisomer and/or a pharmaceutically acceptable salt thereof;
Formula (I6): 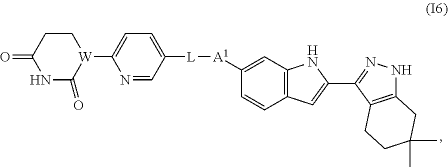
or a stereoisomer and/or a pharmaceutically acceptable salt thereof;
Formula (I7): 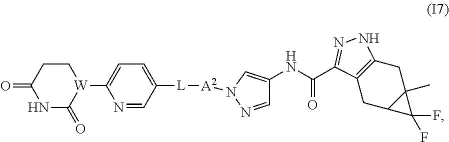
or a stereoisomer and/or a pharmaceutically acceptable salt thereof;
Formula (I7A): 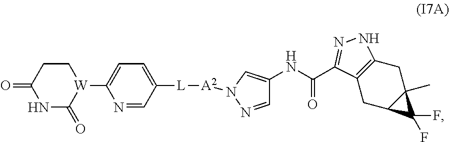
or a stereoisomer and/or a pharmaceutically acceptable salt thereof;
Formula (I8): 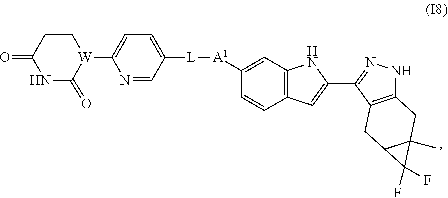
or a stereoisomer and/or a pharmaceutically acceptable salt thereof;
Formula (I8A): 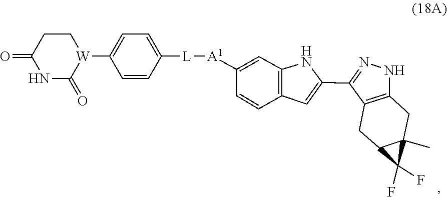
or a stereoisomer and/or a pharmaceutically acceptable salt thereof;
Formula (I9): 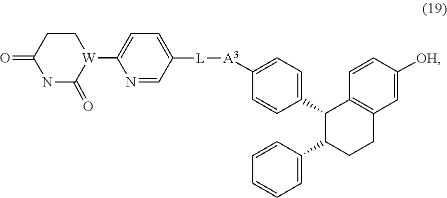
or a stereoisomer and/or a pharmaceutically acceptable salt thereof;
Formula (I10): 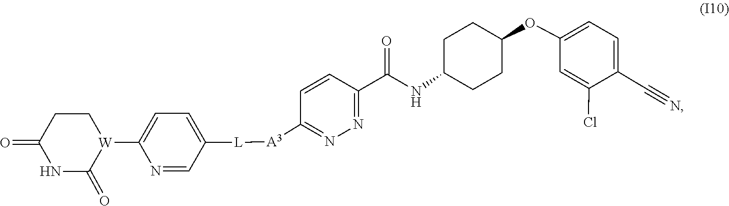
or a stereoisomer and/or a pharmaceutically acceptable salt thereof;
Formula (I11): 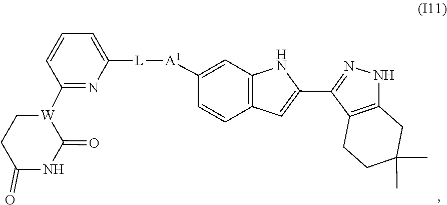
or a stereoisomer and/or a pharmaceutically acceptable salt thereof;
Formula (I12): 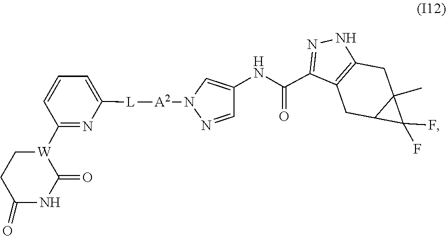
or a stereoisomer and/or a pharmaceutically acceptable salt thereof;
Formula (I12A): 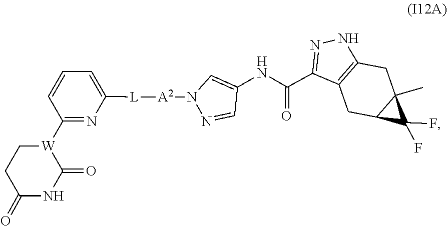
or a stereoisomer and/or a pharmaceutically acceptable salt thereof;
Formula (I13): 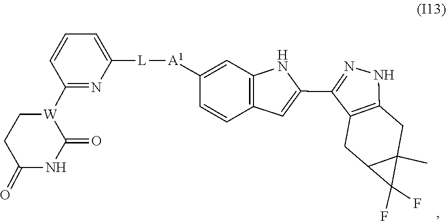
or a stereoisomer and/or a pharmaceutically acceptable salt thereof;
Formula (I13A): 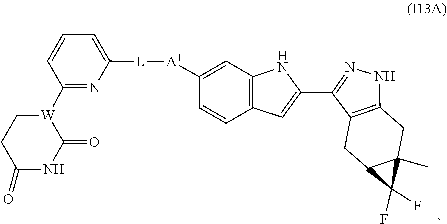
or a stereoisomer and/or a pharmaceutically acceptable salt thereof;
Formula (I14): 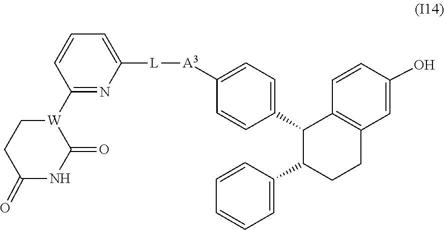
or a stereoisomer and/or a pharmaceutically acceptable salt thereof;
Formula (I15): 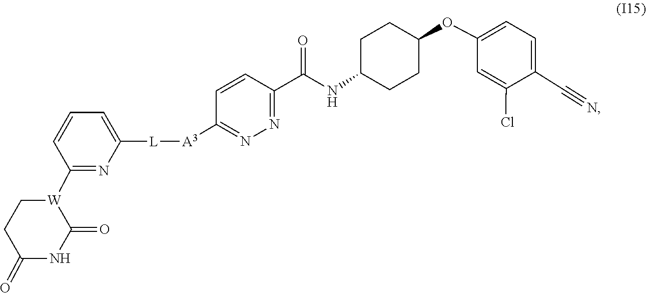
or a stereoisomer and/or a pharmaceutically acceptable salt thereof;
Formula (I16): 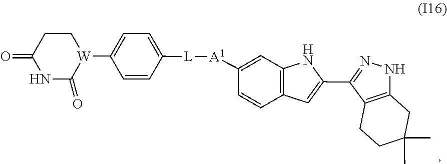
or a stereoisomer and/or a pharmaceutically acceptable salt thereof;
Formula (I17): 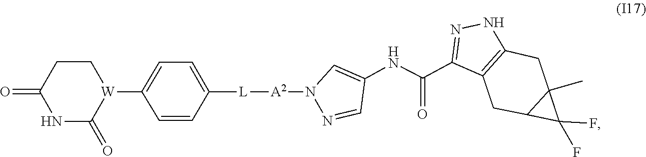
or a stereoisomer and/or a pharmaceutically acceptable salt thereof;
Formula (I17A): 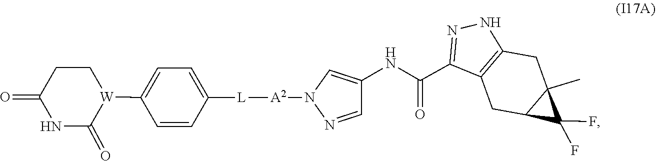
or a stereoisomer and/or a pharmaceutically acceptable salt thereof;
Formula (I18): 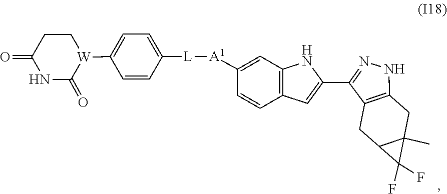
or a stereoisomer and/or a pharmaceutically acceptable salt thereof;
Formula (I18A): 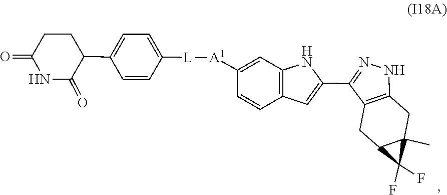
or a stereoisomer and/or a pharmaceutically acceptable salt thereof;
Formula (I19): 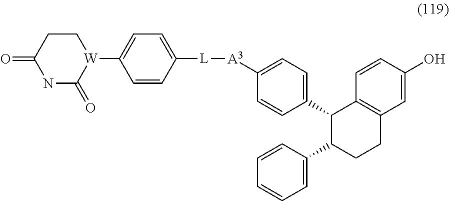
or a stereoisomer and/or a pharmaceutically acceptable salt thereof;
Formula (I20): 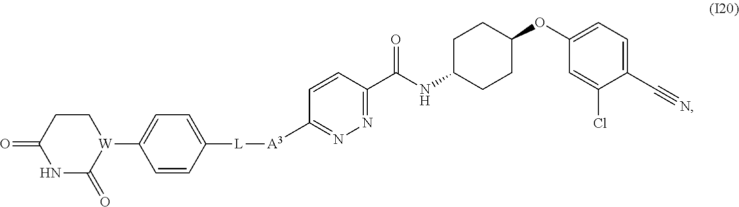
or a stereoisomer and/or a pharmaceutically acceptable salt thereof;
Formula (I21): 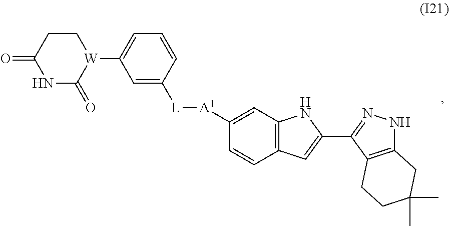
or a stereoisomer and/or a pharmaceutically acceptable salt thereof;
Formula (I22): 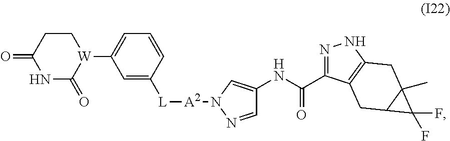
or a stereoisomer and/or a pharmaceutically acceptable salt thereof;
Formula (I22A): 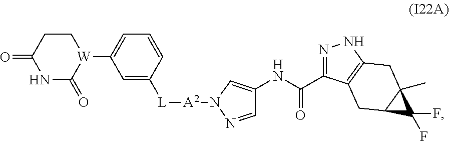
or a stereoisomer and/or a pharmaceutically acceptable salt thereof;
Formula (I23): 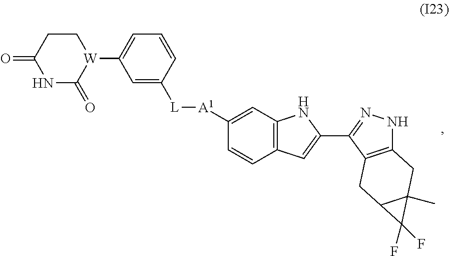
or a stereoisomer and/or a pharmaceutically acceptable salt thereof;
Formula (I23A): 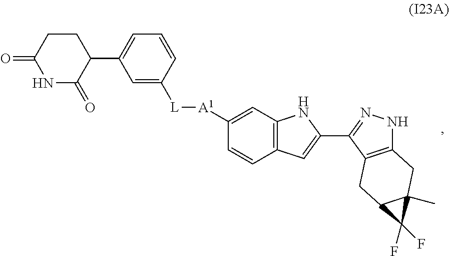
or a stereoisomer and/or a pharmaceutically acceptable salt thereof;
Formula (I24): 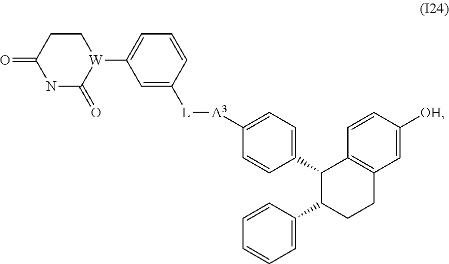
or a stereoisomer and/or a pharmaceutically acceptable salt thereof;
Formula (I25): 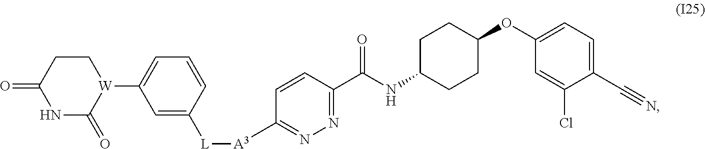
or a stereoisomer and/or a pharmaceutically acceptable salt thereof;
Formula (I26): 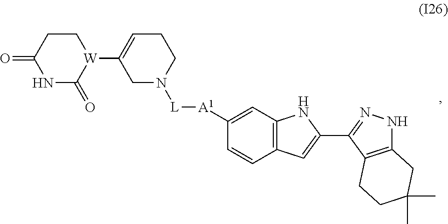
or a stereoisomer and/or a pharmaceutically acceptable salt thereof;
Formula (I27): 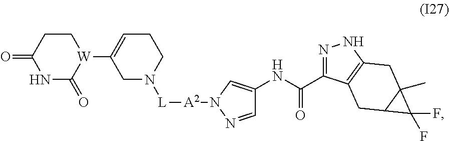
or a stereoisomer and/or a pharmaceutically acceptable salt thereof;
Formula (I27A): 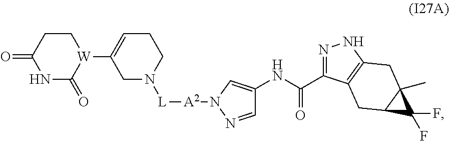
or a stereoisomer and/or a pharmaceutically acceptable salt thereof;
Formula (I28): 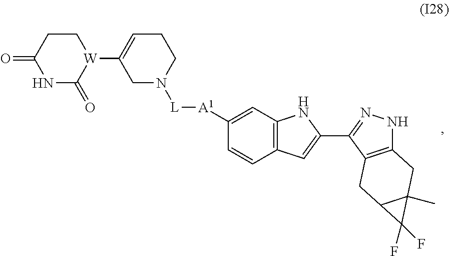
or a stereoisomer and/or a pharmaceutically acceptable salt thereof;
Formula (I28A): 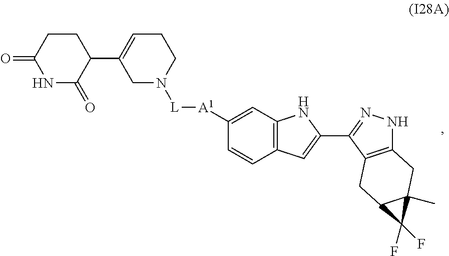
or a stereoisomer and/or a pharmaceutically acceptable salt thereof;
Formula (I29): 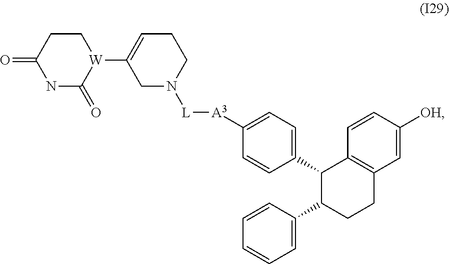
or a stereoisomer and/or a pharmaceutically acceptable salt thereof;
Formula (I30): 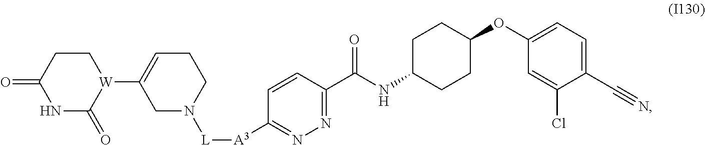
or a stereoisomer and/or a pharmaceutically acceptable salt thereof;
Formula (I31): 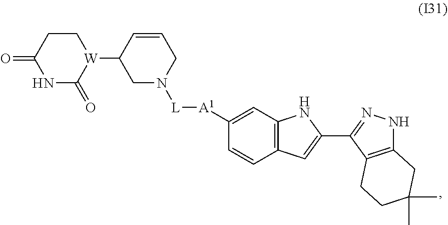
or a stereoisomer and/or a pharmaceutically acceptable salt thereof;
Formula (I32): 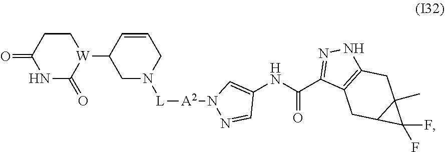
or a stereoisomer and/or a pharmaceutically acceptable salt thereof;
Formula (I32A): 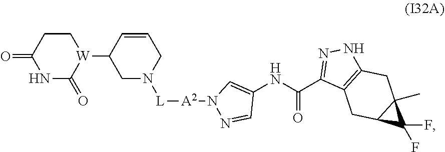
or a stereoisomer and/or a pharmaceutically acceptable salt thereof;
Formula (I33): 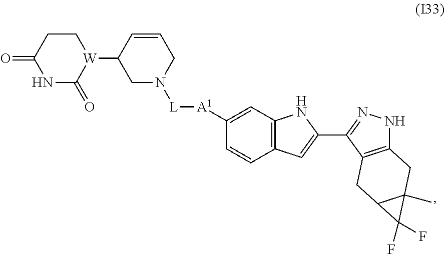
or a stereoisomer and/or a pharmaceutically acceptable salt thereof;
Formula (I33A): 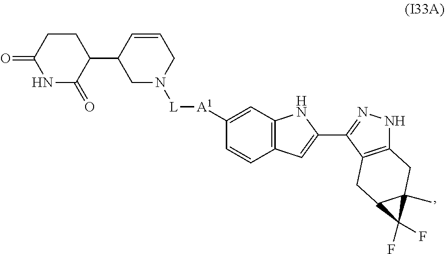
or a stereoisomer and/or a pharmaceutically acceptable salt thereof;
Formula (I34): 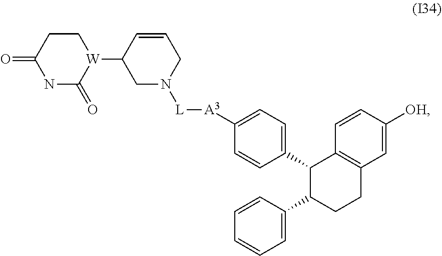
or a stereoisomer and/or a pharmaceutically acceptable salt thereof;
Formula (I35): 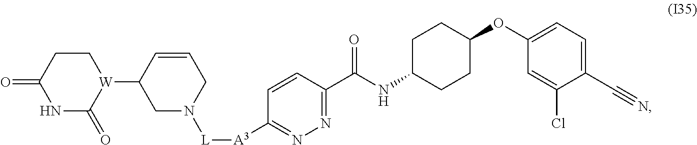
or a stereoisomer and/or a pharmaceutically acceptable salt thereof;
Formula (I36): 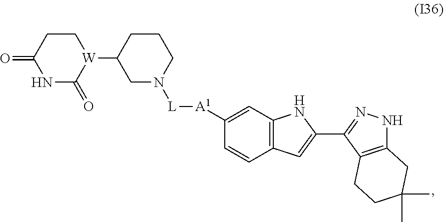
or a stereoisomer and/or a pharmaceutically acceptable salt thereof;
Formula (I37): 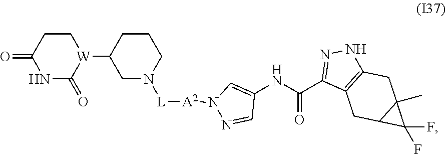
or a stereoisomer and/or a pharmaceutically acceptable salt thereof;
Formula (I37A): 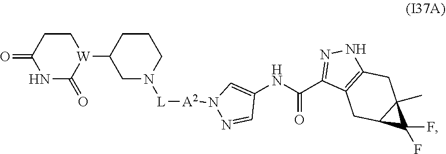
or a stereoisomer and/or a pharmaceutically acceptable salt thereof;
Formula (I38): 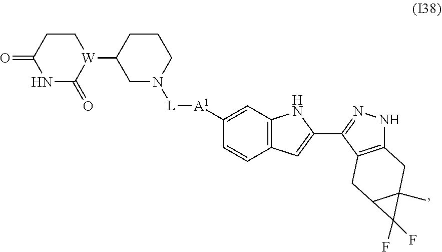
or a stereoisomer and/or a pharmaceutically acceptable salt thereof;
Formula (I38A): 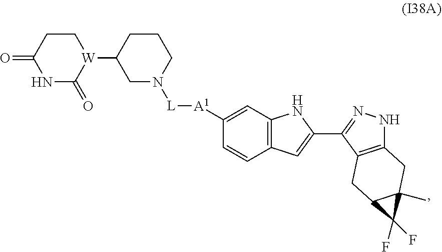
or a stereoisomer and/or a pharmaceutically acceptable salt thereof;
Formula (I39): 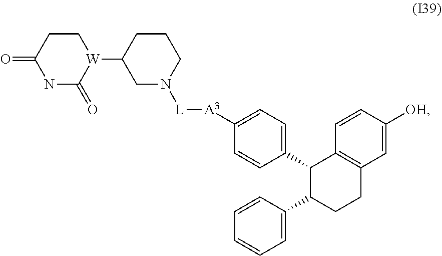
or a stereoisomer and/or a pharmaceutically acceptable salt thereof;
Formula (I40): 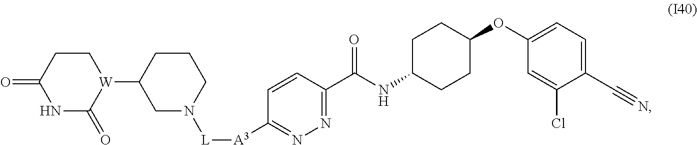
or a stereoisomer and/or a pharmaceutically acceptable salt thereof;
Formula (I41): 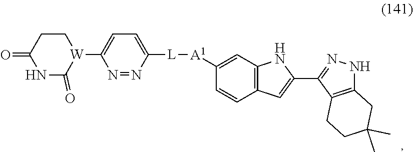
or a stereoisomer and/or a pharmaceutically acceptable salt thereof;
Formula (I142): 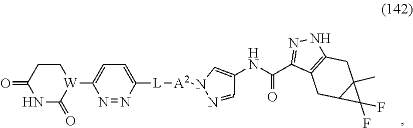
or a stereoisomer and/or a pharmaceutically acceptable salt thereof;
Formula (I42A): 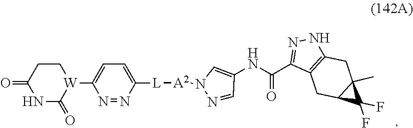
or a stereoisomer and/or a pharmaceutically acceptable salt thereof;
Formula (I43): 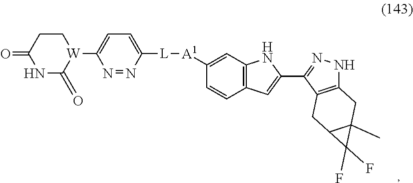
or a stereoisomer and/or a pharmaceutically acceptable salt thereof;
Formula (I43A): 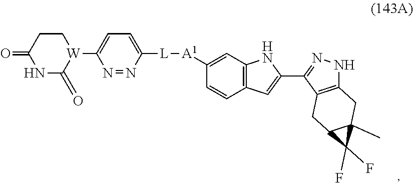
or a stereoisomer and/or a pharmaceutically acceptable salt thereof;
Formula (I44): 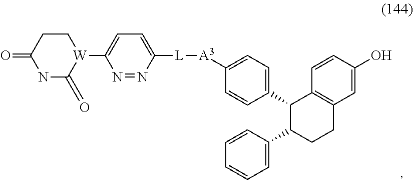
or a stereoisomer and/or a pharmaceutically acceptable salt thereof;
Formula (I45): 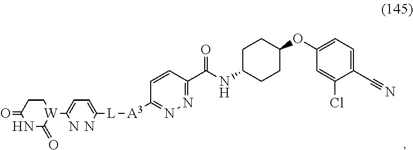
or a stereoisomer and/or a pharmaceutically acceptable salt thereof;
Formula (I46): 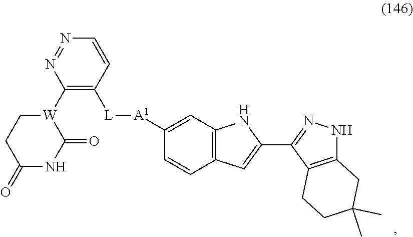
or a stereoisomer and/or a pharmaceutically acceptable salt thereof;
Formula (I47): 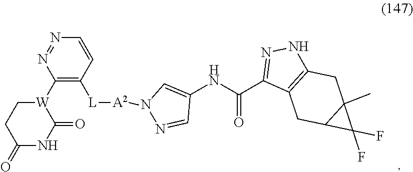
or a stereoisomer and/or a pharmaceutically acceptable salt thereof;
Formula (I47A): 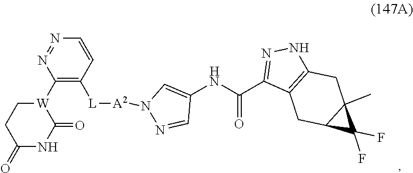
or a stereoisomer and/or a pharmaceutically acceptable salt thereof;
Formula (I48): 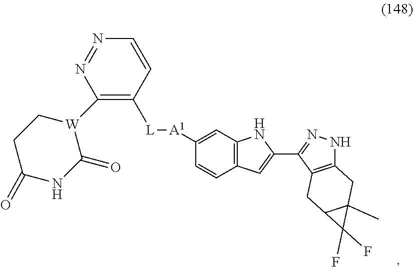
or a stereoisomer and/or a pharmaceutically acceptable salt thereof;
Formula (I48A): 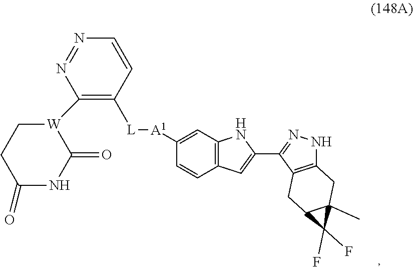
or a stereoisomer and/or a pharmaceutically acceptable salt thereof;
Formula (I49): 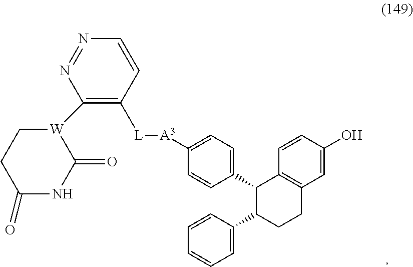
or a stereoisomer and/or a pharmaceutically acceptable salt thereof;
Formula (I50): 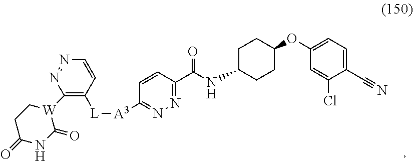
or a stereoisomer and/or a pharmaceutically acceptable salt thereof;
Formula (I51): 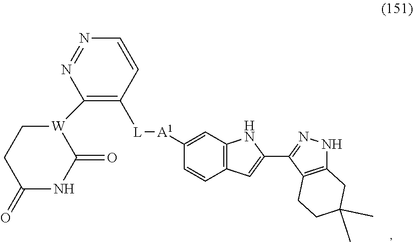
or a stereoisomer and/or a pharmaceutically acceptable salt thereof;
Formula (I52): 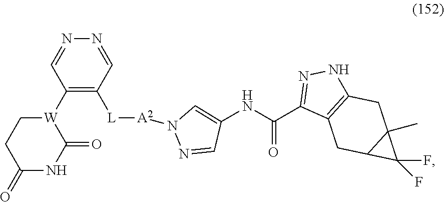
or a stereoisomer and/or a pharmaceutically acceptable salt thereof;
Formula (I52A): 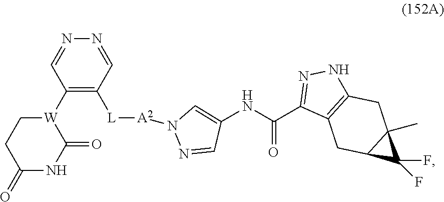
or a stereoisomer and/or a pharmaceutically acceptable salt thereof;
Formula (I53): 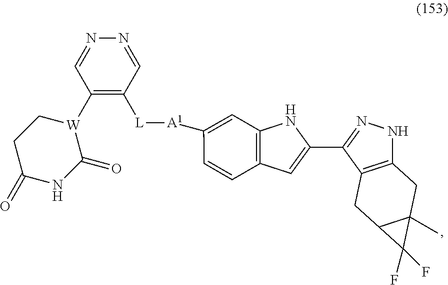
or a stereoisomer and/or a pharmaceutically acceptable salt thereof;
Formula (I53A): 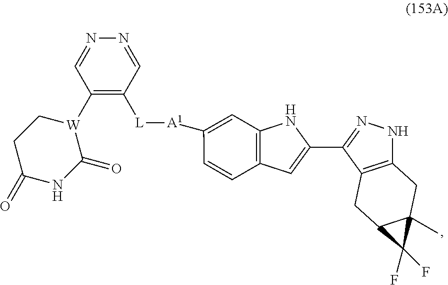
or a stereoisomer and/or a pharmaceutically acceptable salt thereof;
Formula (I54): 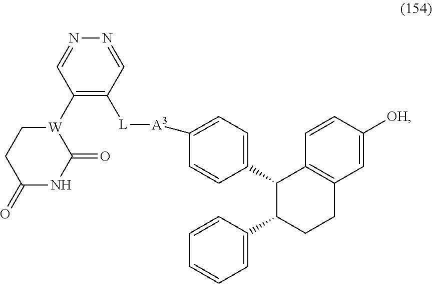
or a stereoisomer and/or a pharmaceutically acceptable salt thereof;
Formula (I55): 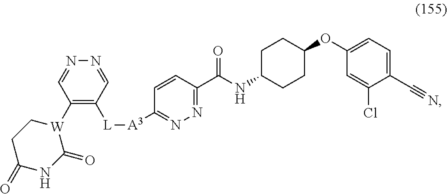
or a stereoisomer and/or a pharmaceutically acceptable salt thereof;
Formula (I56): 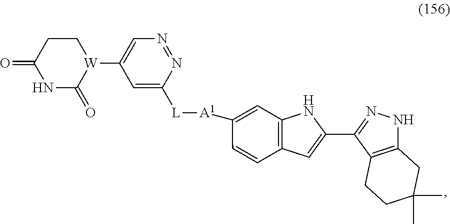
or a stereoisomer and/or a pharmaceutically acceptable salt thereof;
Formula (I57): 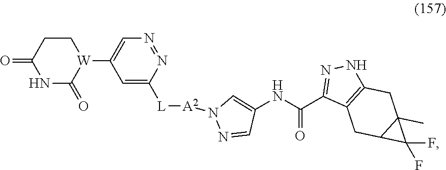
or a stereoisomer and/or a pharmaceutically acceptable salt thereof;
Formula (I57A): 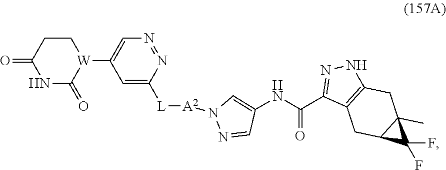
or a stereoisomer and/or a pharmaceutically acceptable salt thereof;
Formula (I58): 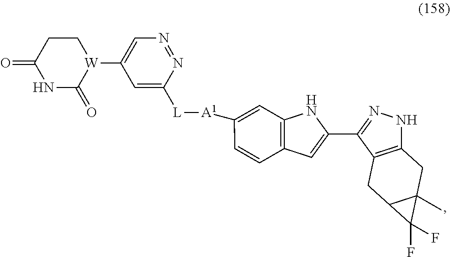
or a stereoisomer and/or a pharmaceutically acceptable salt thereof;
Formula (I58A): 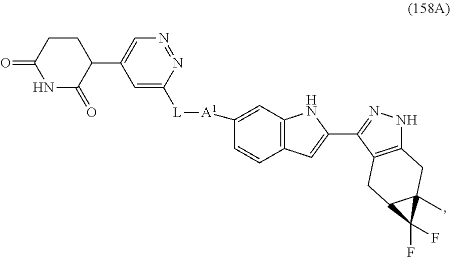
or a stereoisomer and/or a pharmaceutically acceptable salt thereof;
Formula (I59): 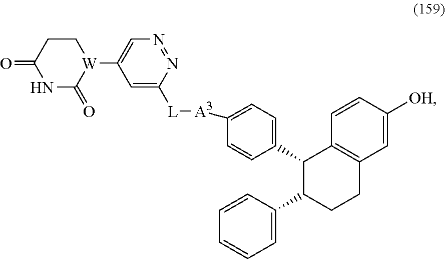
or a stereoisomer and/or a pharmaceutically acceptable salt thereof;
Formula (I60): 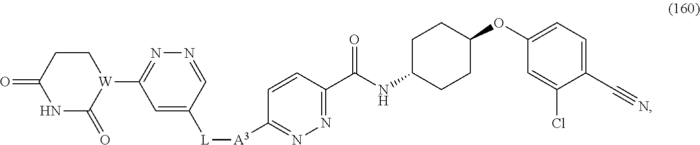
or a stereoisomer and/or a pharmaceutically acceptable salt thereof;
Formula (I61): 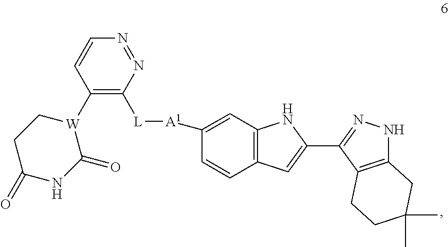
or a stereoisomer and/or a pharmaceutically acceptable salt thereof;
Formula (I62): 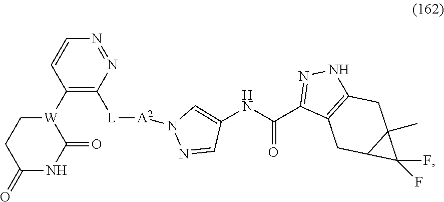
or a stereoisomer and/or a pharmaceutically acceptable salt thereof;
Formula (I62A): 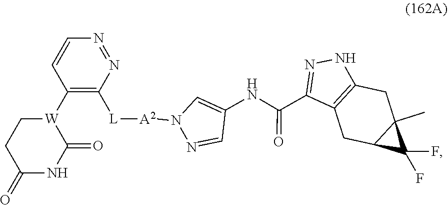
or a stereoisomer and/or a pharmaceutically acceptable salt thereof;
Formula (I63): 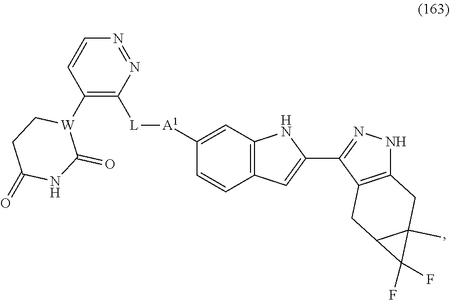
or a stereoisomer and/or a pharmaceutically acceptable salt thereof;
Formula (I63A): 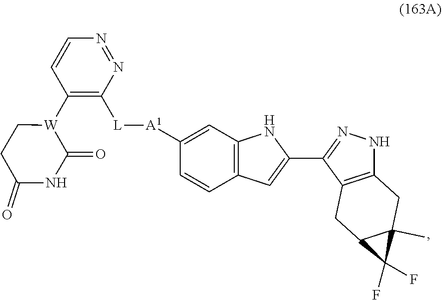
or a stereoisomer and/or a pharmaceutically acceptable salt thereof;
Formula (I64): 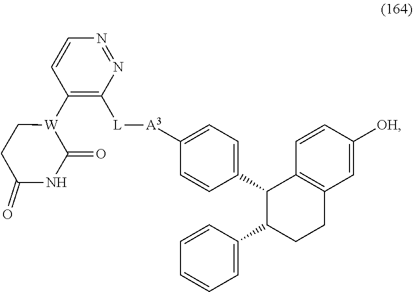
or a stereoisomer and/or a pharmaceutically acceptable salt thereof;
Formula (I65): 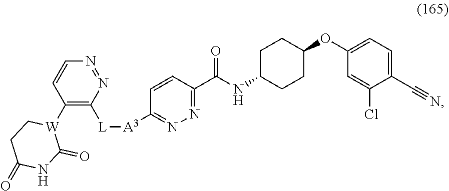
or a stereoisomer and/or a pharmaceutically acceptable salt thereof;
Formula (I66): 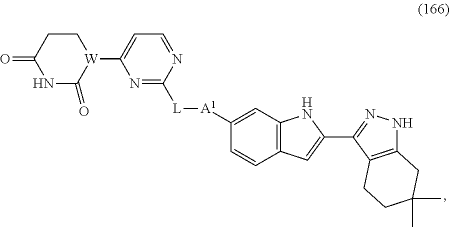
or a stereoisomer and/or a pharmaceutically acceptable salt thereof;
Formula (I67): 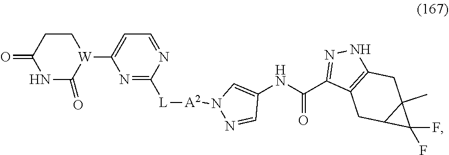
or a stereoisomer and/or a pharmaceutically acceptable salt thereof;
Formula (I67A): 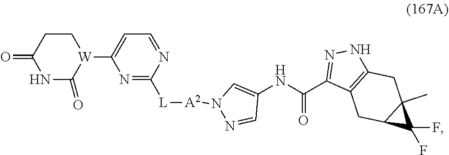
or a stereoisomer and/or a pharmaceutically acceptable salt thereof;
Formula (I68): 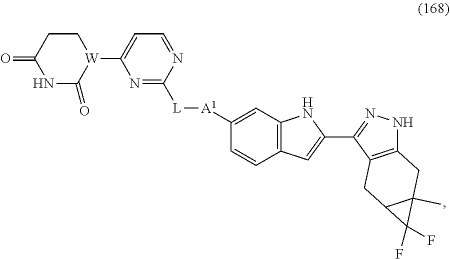
or a stereoisomer and/or a pharmaceutically acceptable salt thereof;
Formula (I68A): 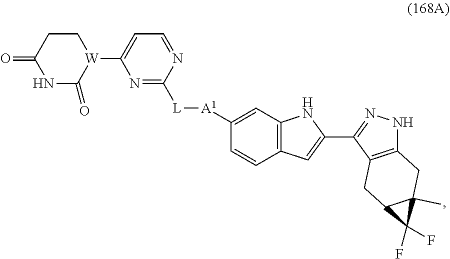
or a stereoisomer and/or a pharmaceutically acceptable salt thereof;
Formula (I69): 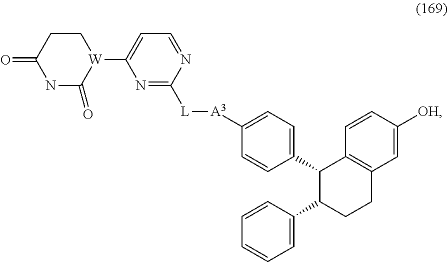
or a stereoisomer and/or a pharmaceutically acceptable salt thereof;
Formula (I70) 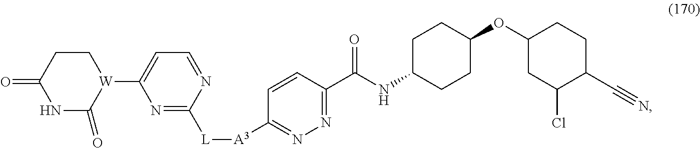
or a stereoisomer and/or a pharmaceutically acceptable salt thereof;
Formula (I71): 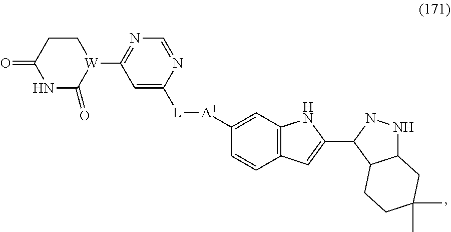
or a stereoisomer and/or a pharmaceutically acceptable salt thereof;
Formula (I72): 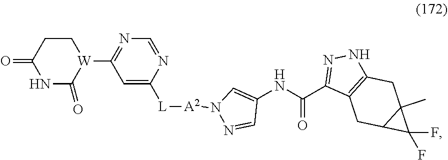
or a stereoisomer and/or a pharmaceutically acceptable salt thereof;
Formula (I72A): 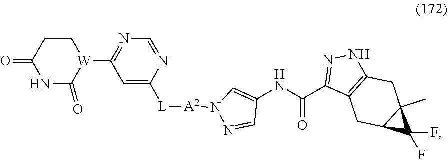
or a stereoisomer and/or a pharmaceutically acceptable salt thereof;
Formula (I73): 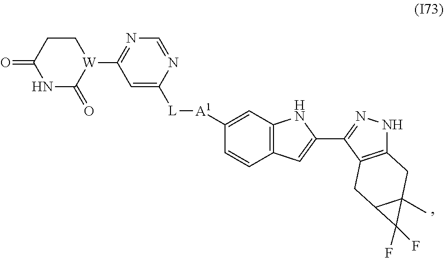
or a stereoisomer and/or a pharmaceutically acceptable salt thereof;
Formula (I73A): 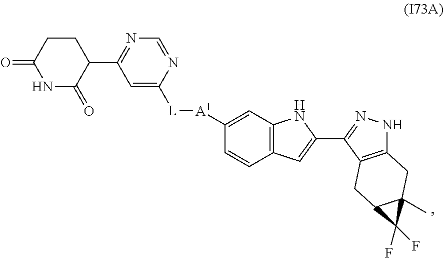
or a stereoisomer and/or a pharmaceutically acceptable salt thereof;
Formula (I74): 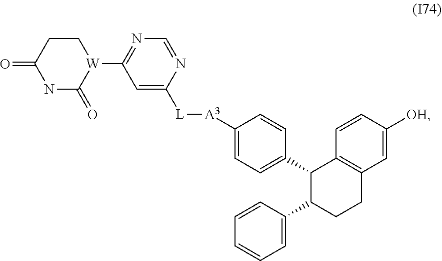
or a stereoisomer and/or a pharmaceutically acceptable salt thereof;
Formula (I75): 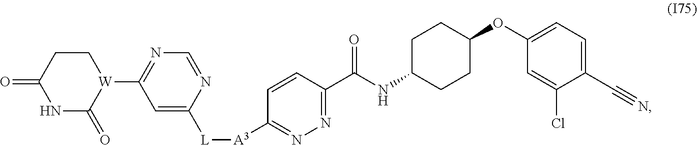
or a stereoisomer and/or a pharmaceutically acceptable salt thereof;
Formula (I76): 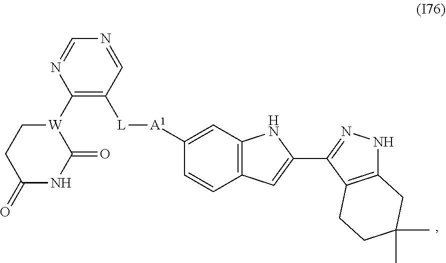
or a stereoisomer and/or a pharmaceutically acceptable salt thereof;
Formula (I77): 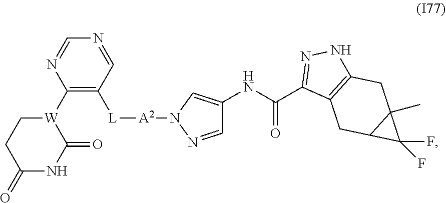
or a stereoisomer and/or a pharmaceutically acceptable salt thereof;
Formula (I77A): 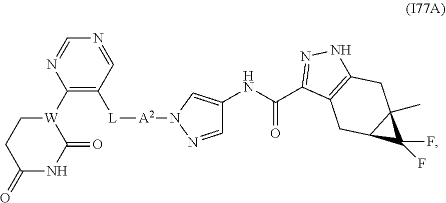
or a stereoisomer and/or a pharmaceutically acceptable salt thereof;
Formula (I78): 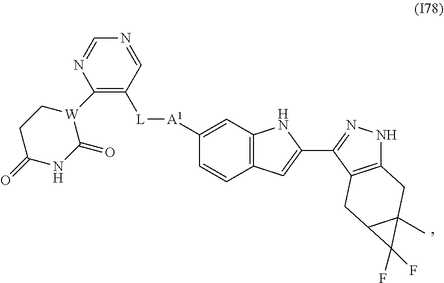
or a stereoisomer and/or a pharmaceutically acceptable salt thereof;
Formula (I78A): 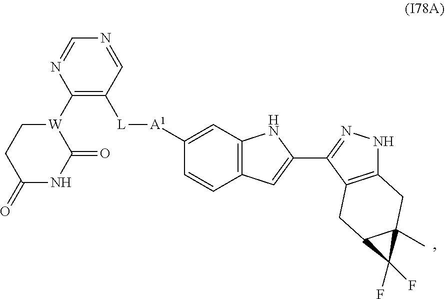
or a stereoisomer and/or a pharmaceutically acceptable salt thereof;
Formula (I79): 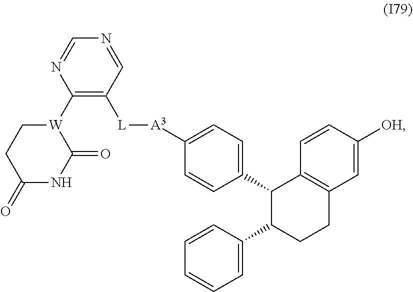
or a stereoisomer and/or a pharmaceutically acceptable salt thereof;
Formula (I80): 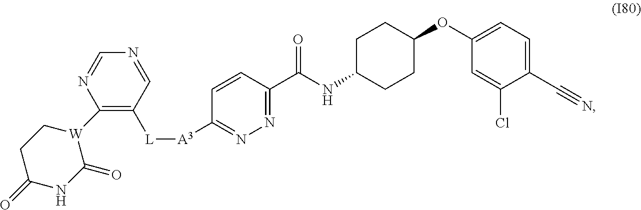
or a stereoisomer and/or a pharmaceutically acceptable salt thereof;
Formula (I81): 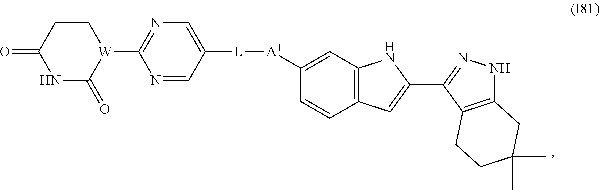
or a stereoisomer and/or a pharmaceutically acceptable salt thereof;
Formula (I82): 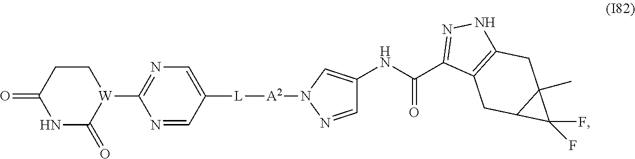
or a stereoisomer and/or a pharmaceutically acceptable salt thereof;
Formula (I82A): 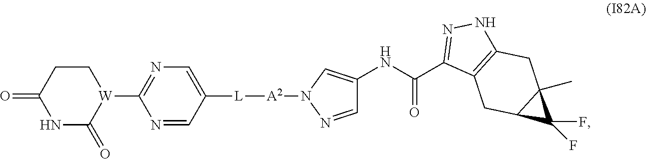
or a stereoisomer and/or a pharmaceutically acceptable salt thereof;
Formula (I83): 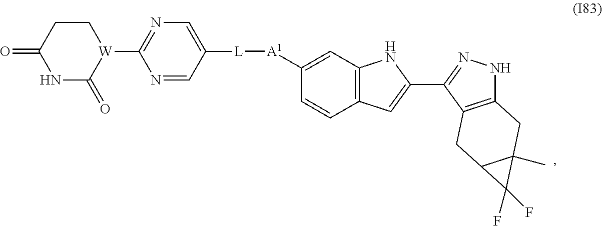
or a stereoisomer and/or a pharmaceutically acceptable salt thereof;
Formula (I83A): 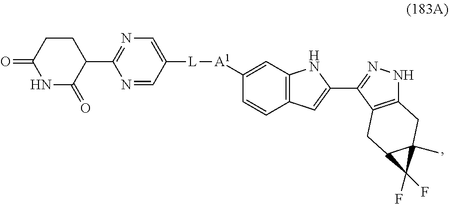
or a stereoisomer and/or a pharmaceutically acceptable salt thereof;
Formula (I84): 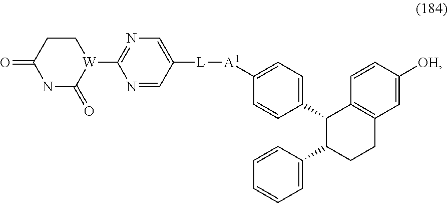
or a stereoisomer and/or a pharmaceutically acceptable salt thereof;
Formula (I85): 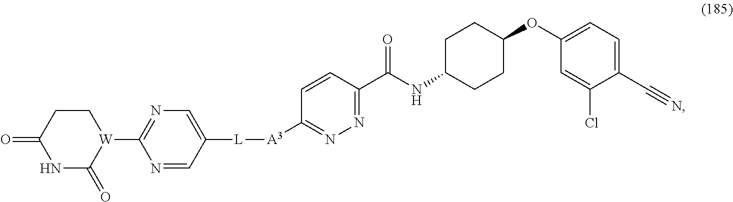
or a stereoisomer and/or a pharmaceutically acceptable salt thereof;
Formula (I86): 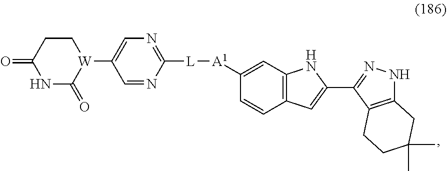
or a stereoisomer and/or a pharmaceutically acceptable salt thereof;
Formula (I87): 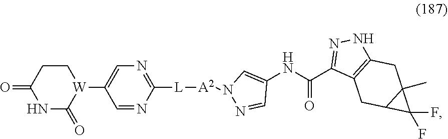
or a stereoisomer and/or a pharmaceutically acceptable salt thereof;
Formula (I87A): 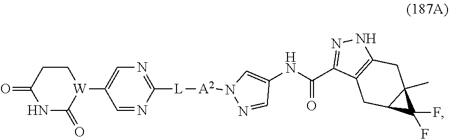
or a stereoisomer and/or a pharmaceutically acceptable salt thereof;
Formula (I88): 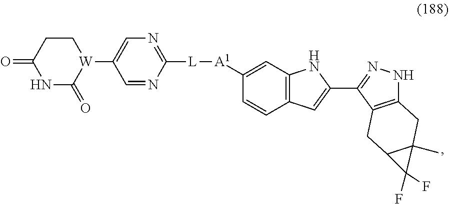
or a stereoisomer and/or a pharmaceutically acceptable salt thereof;
Formula (I88A): 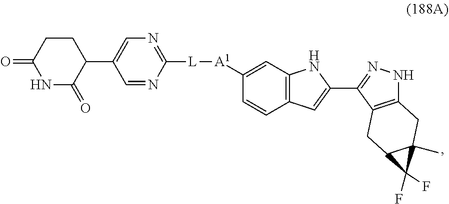
or a stereoisomer and/or a pharmaceutically acceptable salt thereof;
Formula (I89): 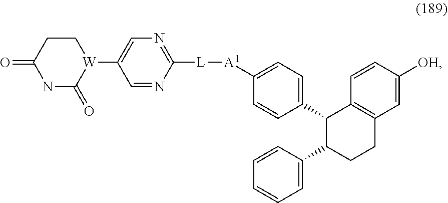
or a stereoisomer and/or a pharmaceutically acceptable salt thereof;
Formula (I90): 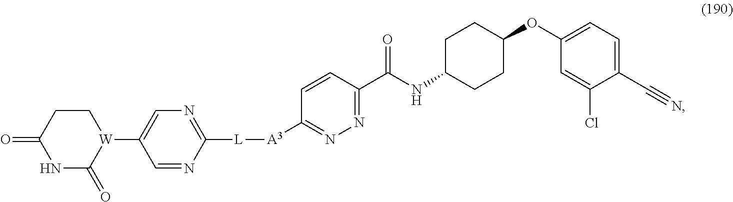
or a stereoisomer and/or a pharmaceutically acceptable salt thereof;
Formula (I91): 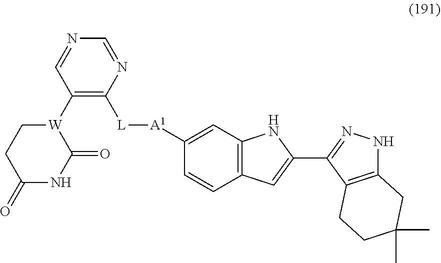
or a stereoisomer and/or a pharmaceutically acceptable salt thereof;
Formula (I92): 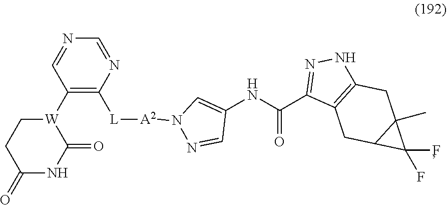
or a stereoisomer and/or a pharmaceutically acceptable salt thereof;
Formula (I92A): 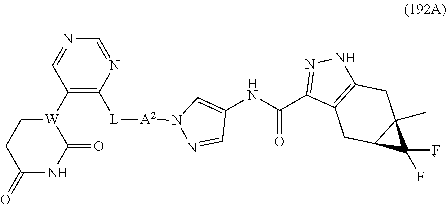
or a stereoisomer and/or a pharmaceutically acceptable salt thereof;
Formula (I93): 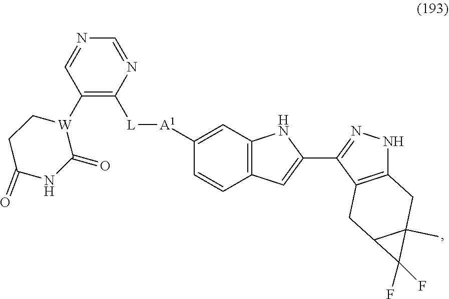
or a stereoisomer and/or a pharmaceutically acceptable salt thereof;
Formula (I93A): 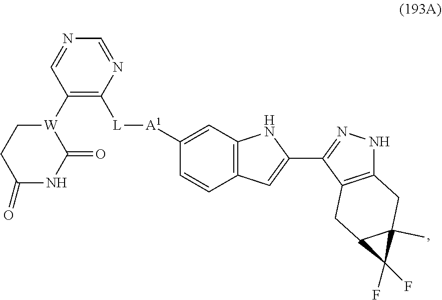
or a stereoisomer and/or a pharmaceutically acceptable salt thereof;
Formula (I94): 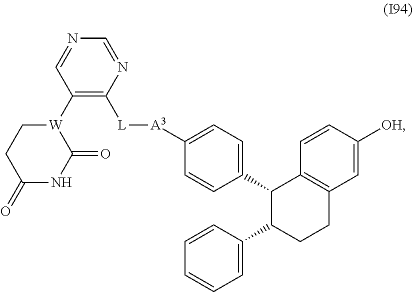
or a stereoisomer and/or a pharmaceutically acceptable salt thereof;
Formula (I95): 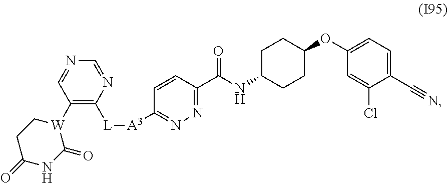
or a stereoisomer and/or a pharmaceutically acceptable salt thereof;
Formula (I96): 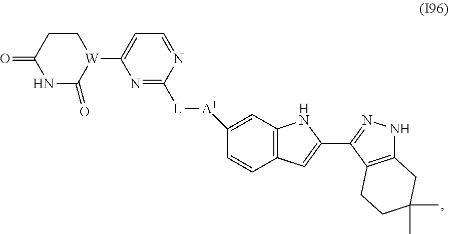
or a stereoisomer and/or a pharmaceutically acceptable salt thereof;
Formula (I97): 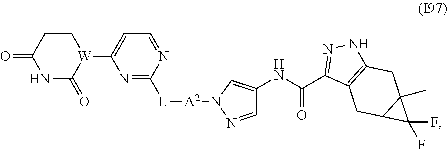
or a stereoisomer and/or a pharmaceutically acceptable salt thereof;
Formula (I97A): 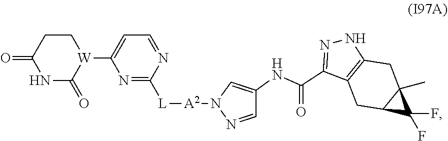
or a stereoisomer and/or a pharmaceutically acceptable salt thereof;
Formula (I98): 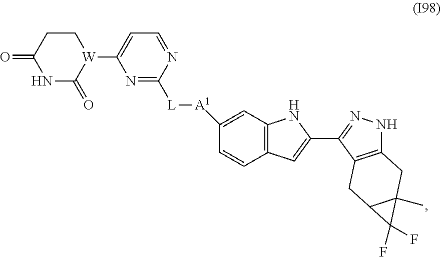
or a stereoisomer and/or a pharmaceutically acceptable salt thereof;
Formula (I98A): 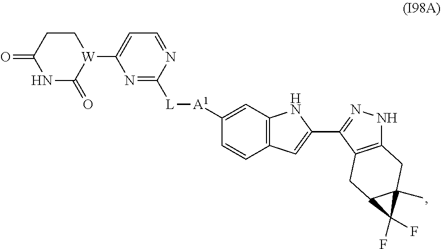
or a stereoisomer and/or a pharmaceutically acceptable salt thereof;
Formula (I99): 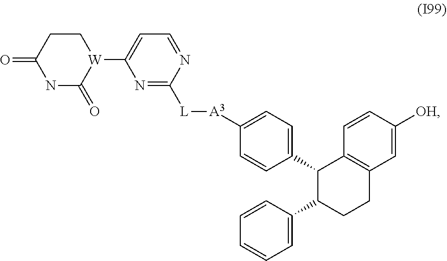
or a stereoisomer and/or a pharmaceutically acceptable salt thereof;
Formula (I100): 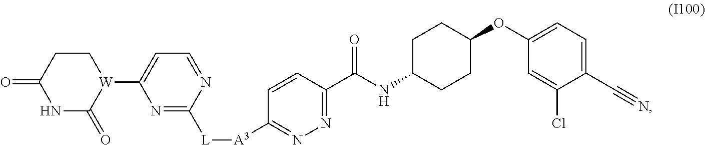
or a stereoisomer and/or a pharmaceutically acceptable salt thereof;
Formula (I101): 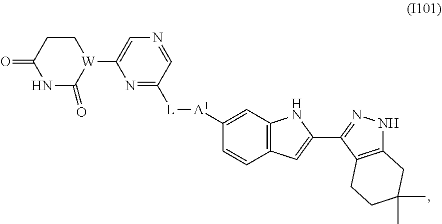
or a stereoisomer and/or a pharmaceutically acceptable salt thereof;
Formula (I102): 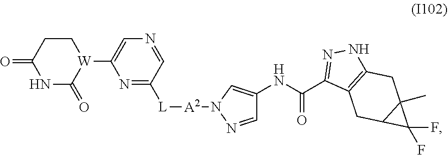
or a stereoisomer and/or a pharmaceutically acceptable salt thereof;
Formula (I102A): 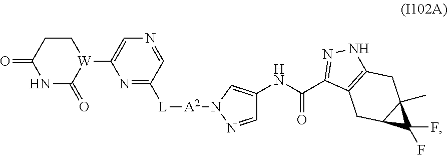
or a stereoisomer and/or a pharmaceutically acceptable salt thereof;
Formula (I103): 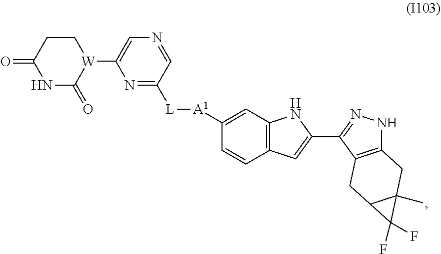
or a stereoisomer and/or a pharmaceutically acceptable salt thereof;
Formula (I103A): 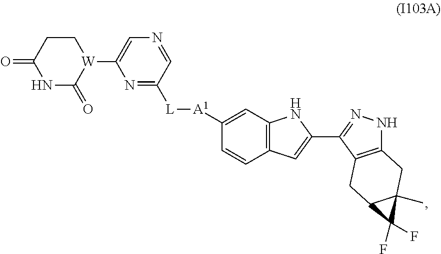
or a stereoisomer and/or a pharmaceutically acceptable salt thereof;
Formula (I104): 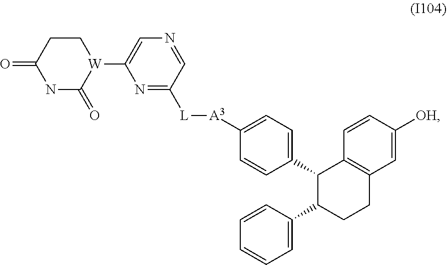
or a stereoisomer and/or a pharmaceutically acceptable salt thereof;
Formula (I105): 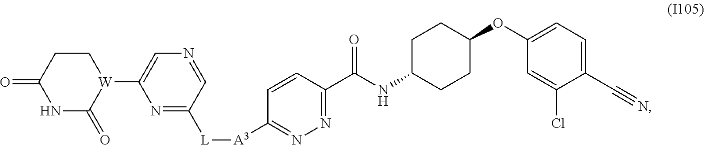
or a stereoisomer and/or a pharmaceutically acceptable salt thereof;
Formula (I106): 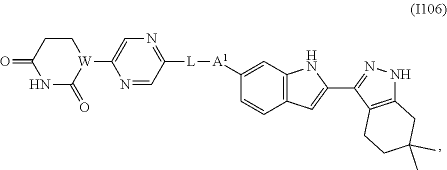
or a stereoisomer and/or a pharmaceutically acceptable salt thereof;
Formula (I107): 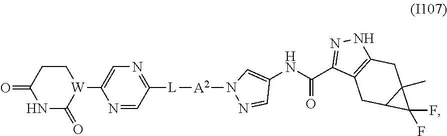
or a stereoisomer and/or a pharmaceutically acceptable salt thereof;
Formula (I107A): 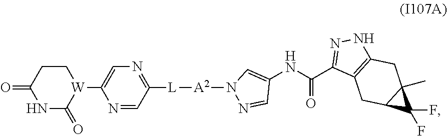
or a stereoisomer and/or a pharmaceutically acceptable salt thereof;
Formula (I108): 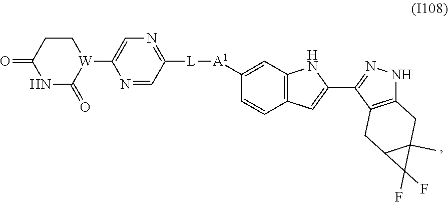
or a stereoisomer and/or a pharmaceutically acceptable salt thereof;
Formula (I108A): 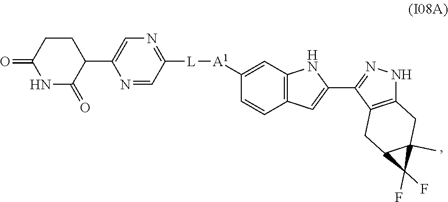
or a stereoisomer and/or a pharmaceutically acceptable salt thereof;
Formula (I109): 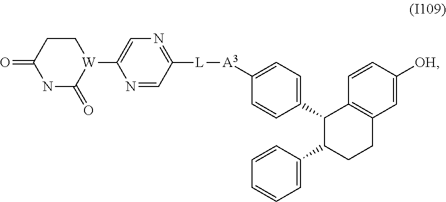
or a stereoisomer and/or a pharmaceutically acceptable salt thereof;
Formula (I110): 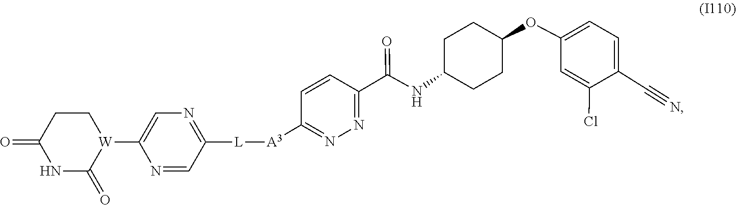
or a stereoisomer and/or a pharmaceutically acceptable salt thereof;
Formula (I111): 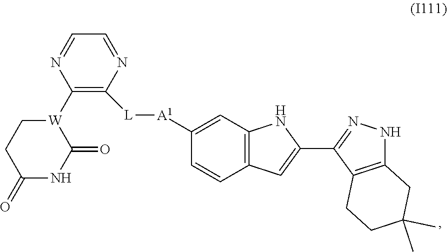
or a stereoisomer and/or a pharmaceutically acceptable salt thereof;
Formula (I112): 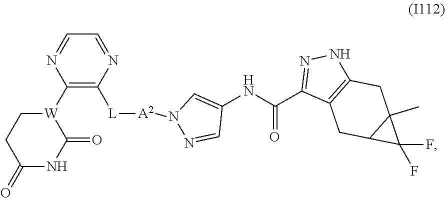
or a stereoisomer and/or a pharmaceutically acceptable salt thereof;
Formula (I112A): 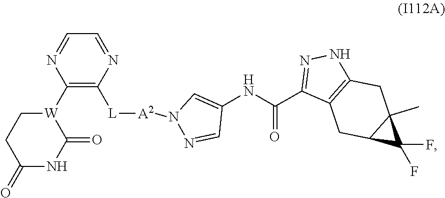
or a stereoisomer and/or a pharmaceutically acceptable salt thereof;
Formula (I113): 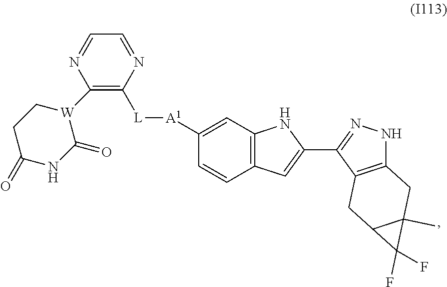
or a stereoisomer and/or a pharmaceutically acceptable salt thereof
Formula (I113A): 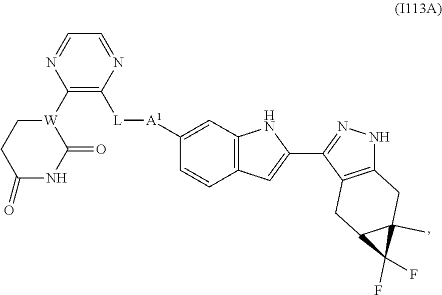
or a stereoisomer and/or a pharmaceutically acceptable salt thereof;
Formula (I114): 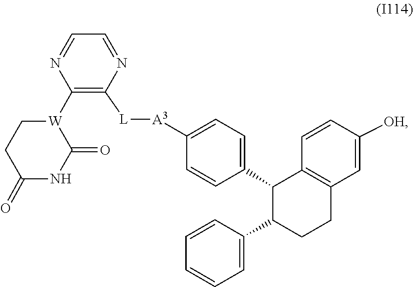
or a stereoisomer and/or a pharmaceutically acceptable salt thereof; and
Formula (I115): 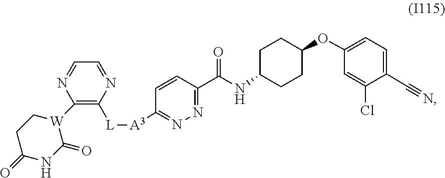
or a stereoisomer and/or a pharmaceutically acceptable salt thereof.

78.-246. (canceled)

247. The compound of claim 1 selected from Table 1, or a stereoisomer and/or a pharmaceutically acceptable salt thereof:
TABLE 1

| Cpd. No. | Structure |
|---|---|
| 1 | 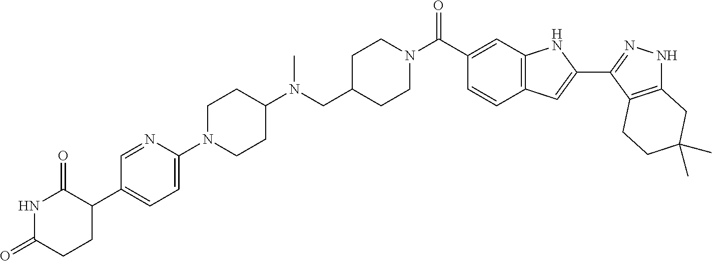 [SMILES: CN(CC1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1] |
| 2 | 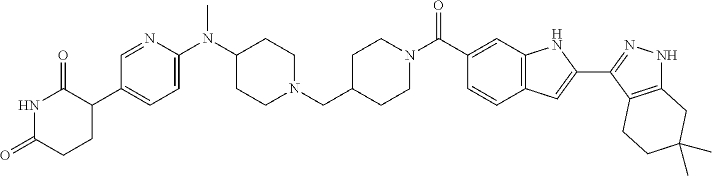 [SMILES: CN(c1ccc(C2CCC(=O)NC2=O)cn1)C1CCN(CC2CCN(C(=O)c3ccc4cc(-c5n[nH]c6c5CCC(C)(C)C6)[nH]c4c3)CC2)CC1] |
| 3 | 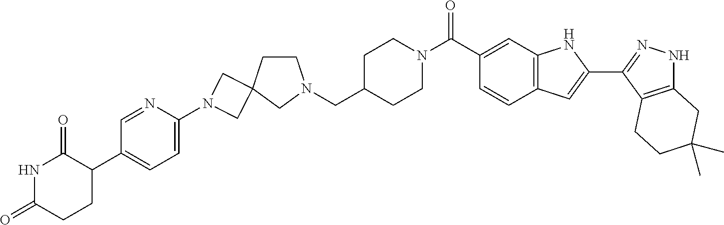 [SMILES: CC1(C)CCc2c(-c3cc4ccc(C(=O)N5CCC(CN6CCC7(C6)CN(c6ccc(C8CCC(=O)NC8=O)cn6)C7)CC5)cc4[nH]3)n[nH]c2C1] |
| 4 | 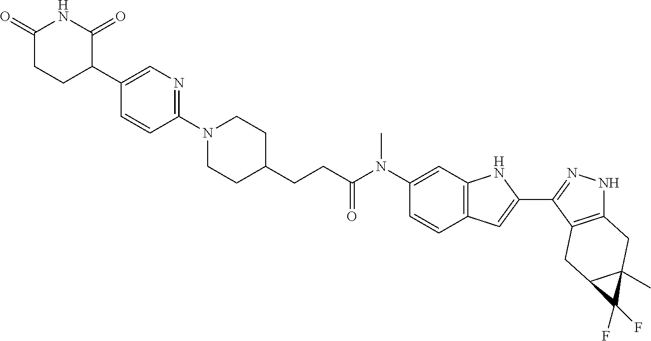 [SMILES: CN(C(=O)CCC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1] |
| 5 | 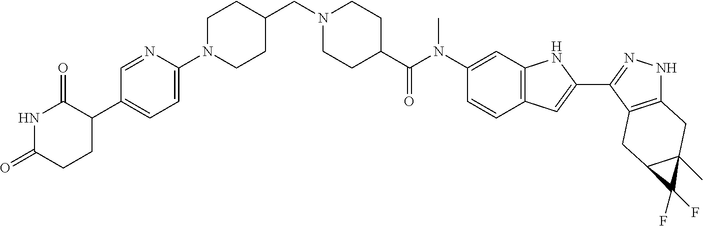 [SMILES: CN(C(=O)C1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1] |
| 6 | 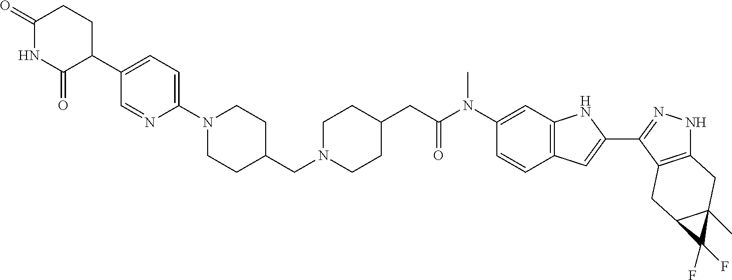 [SMILES: CN(C(=O)CC1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1] |
| 7 | 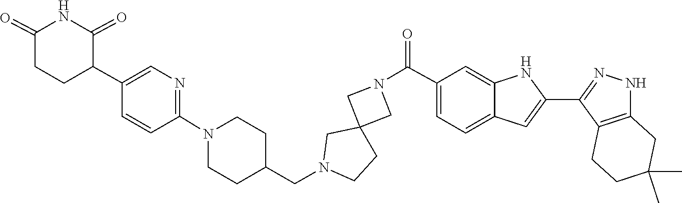 [SMILES: CC1(C)CCc2c(-c3cc4ccc(C(=O)N5CC6(CCN(CC7CCN(c8ccc(C9CCC(=O)NC9=O)cn8)CC7)C6)C5)cc4[nH]3)n[nH]c2C1] |
| 8 | 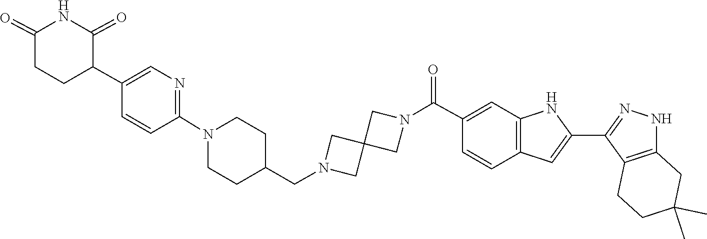 [SMILES: CC1(C)CCc2c(-c3cc4ccc(C(=O)N5CC6(CN(CC7CCN(c8ccc(C9CCC(=O)NC9=O)cn8)CC7)C6)C5)cc4[nH]3)n[nH]c2C1] |
| 9 | 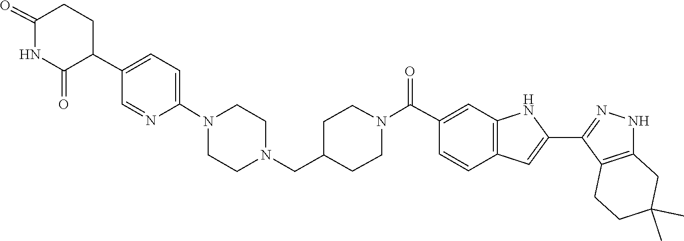 [SMILES: CC1(C)CCc2c(-c3cc4ccc(C(=O)N5CCC(CN6CCN(c7ccc(C8CCC(=O)NC8=O)cn7)CC6)CC5)cc4[nH]3)n[nH]c2C1] |
| 10 | 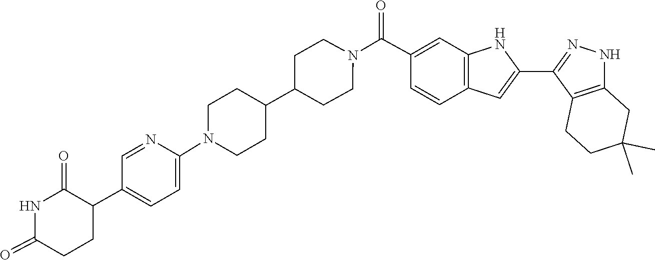 [SMILES: CC1(C)CCc2c(-c3cc4ccc(C(=O)N5CCC(C6CCN(c7ccc(C8CCC(=O)NC8=O)cn7)CC6)CC5)cc4[nH]3)n[nH]c2C1] |
| 11 | 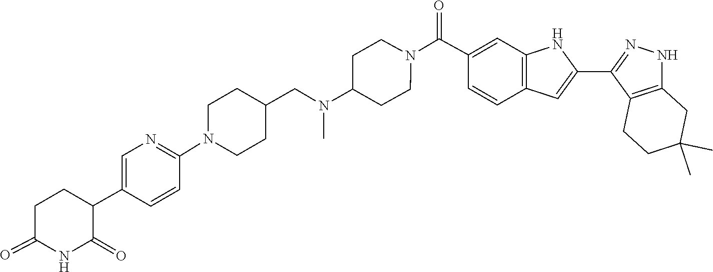 [SMILES: CN(CC1CCN(c2ccc(C3CCC(=O)NC3=O)cn2)CC1)C1CCN(C(=O)c2ccc3cc(-c4n[nH]c5c4CCC(C)(C)C5)[nH]c3c2)CC1] |
| 12 | 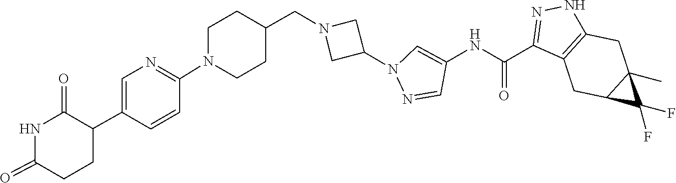 [SMILES: C[C@@]12Cc3[nH]nc(C(=O)Nc4cnn(C5CN(CC6CCN(c7ccc(C8CCC(=O)NC8=O)cn7)CC6)C5)c4)c3C[C@@H]1C2(F)F] |
| 13 | 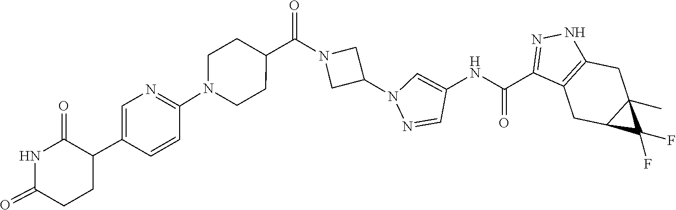 [SMILES: C[C@@]12Cc3[nH]nc(C(=O)Nc4cnn(C5CN(C(=O)C6CCN(c7ccc(C8CCC(=O)NC8=O)cn7)CC6)C5)c4)c3C[C@@H]1C2(F)F] |
| 14 | 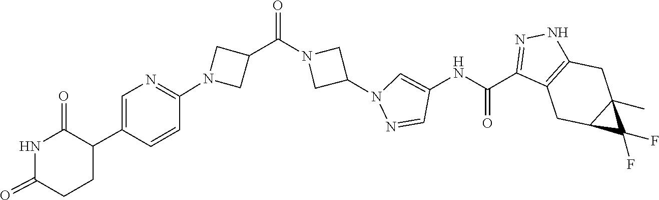 [SMILES: C[C@@]12Cc3[nH]nc(C(=O)Nc4cnn(C5CN(C(=O)C6CN(c7ccc(C8CCC(=O)NC8=O)cn7)C6)C5)c4)c3C[C@@H]1C2(F)F] |
| 15 | 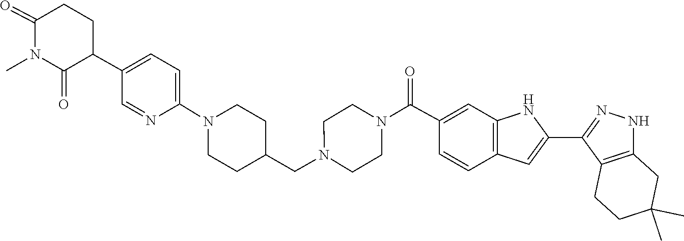 [SMILES: CN1C(=O)CCC(c2ccc(N3CCC(CN4CCN(C(=O)c5ccc6cc(-c7n[nH]c8c7CCC(C)(C)C8)[nH]c6c5)CC4)CC3)nc2)C1=O] |
| 16 | 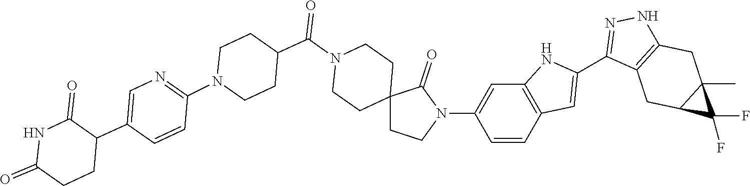 [SMILES: C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F] |
| 17 | 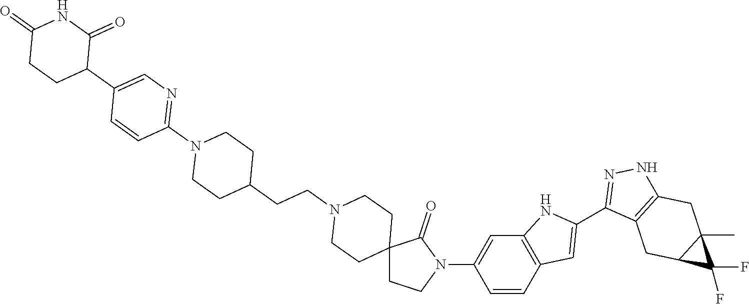 [SMILES: C[C@@]12Cc3[nH]nc(-c4cc5ccc(N6CCC7(CCN(CCC8CCN(c9ccc(C%10CCC(=O)NC%10=O)cn9)CC8)CC7)C6=O)cc5[nH]4)c3C[C@@H]1C2(F)F] |
| 18 | 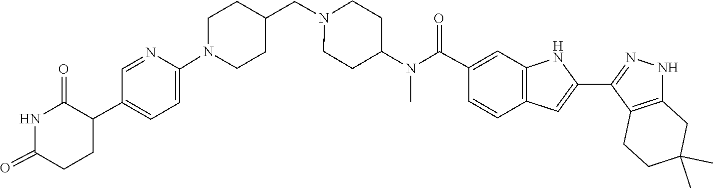 [SMILES: CN(C(=O)c1ccc2cc(-c3n[nH]c4c3CCC(C)(C)C4)[nH]c2c1)C1CCN(CC2CCN(c3ccc(C4CCC(=O)NC4=O)cn3)CC2)CC1] |
| 19 | 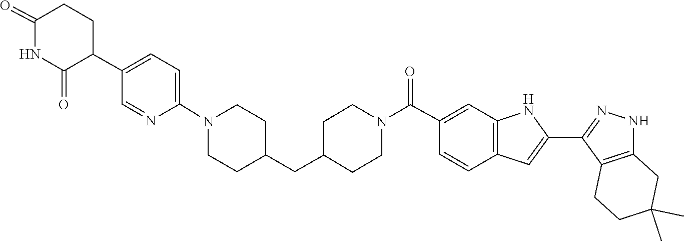 [SMILES: CC1(C)CCc2c(-c3cc4ccc(C(=O)N5CCC(CC6CCN(c7ccc(C8CCC(=O)NC8=O)cn7)CC6)CC5)cc4[nH]3)n[nH]c2C1] |
| 20 | 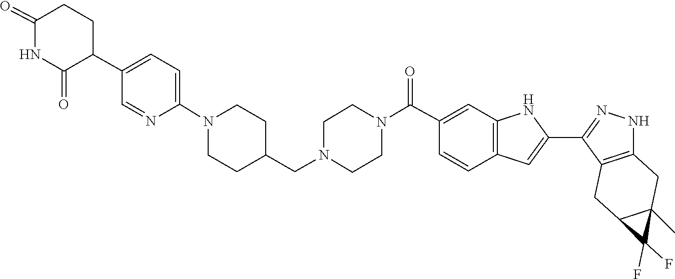 [SMILES: C[C@@]12Cc3[nH]nc(-c4cc5ccc(C(=O)N6CCN(CC7CCN(c8ccc(C9CCC(=O)NC9=O)cn8)CC7)CC6)cc5[nH]4)c3C[C@@H]1C2(F)F] |
| 21 | 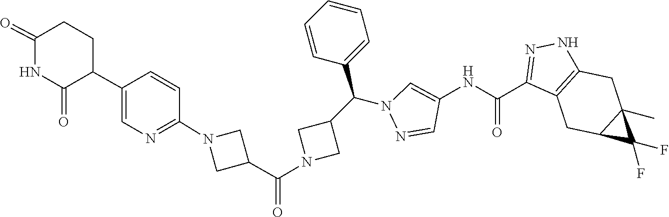 [SMILES: C[C@@]12Cc3[nH]nc(C(=O)Nc4cnn([C@H](c5ccccc5)C5CN(C(=O)C6CN(c7ccc(C8CCC(=O)NC8=O)cn7)C6)C5)c4)c3C[C@@H]1C2(F)F] |
| 22 | 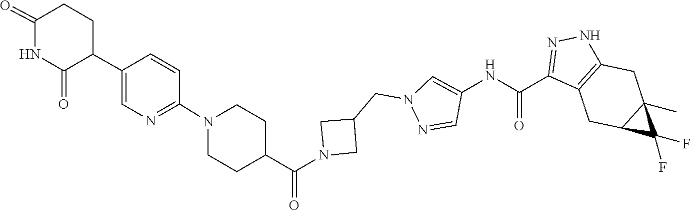 [SMILES: C[C@@]12Cc3[nH]nc(C(=O)Nc4cnn(CC5CN(C(=O)C6CCN(c7ccc(C8CCC(=O)NC8=O)cn7)CC6)C5)c4)c3C[C@@H]1C2(F)F] |
| 23 | 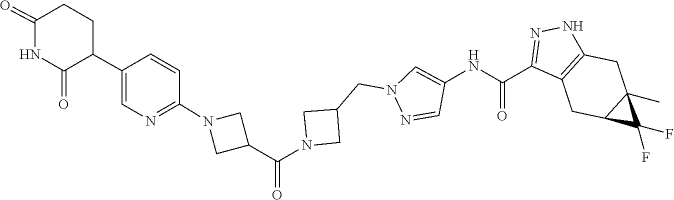 [SMILES: C[C@@]12Cc3[nH]nc(C(=O)Nc4cnn(CC5CN(C(=O)C6CN(c7ccc(C8CCC(=O)NC8=O)cn7)C6)C5)c4)c3C[C@@H]1C2(F)F] |
| 24 | 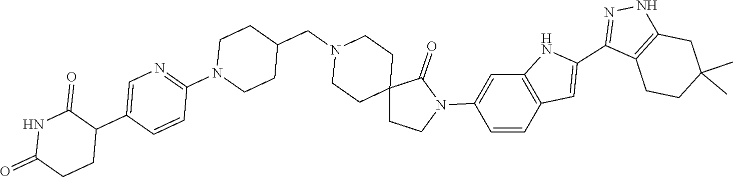 [SMILES: CC1(C)CCc2c(-c3cc4ccc(N5CCC6(CCN(CC7CCN(c8ccc(C9CCC(=O)NC9=O)cn8)CC7)CC6)C5=O)cc4[nH]3)n[nH]c2C1] |
| 25 | 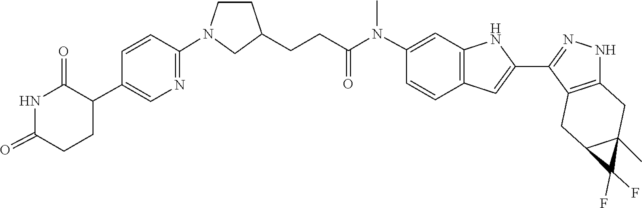 [SMILES: CN(C(=O)CCC1CCN(c2ccc(C3CCC(=O)NC3=O)cn2)C1)c1ccc2cc(-c3n[nH]c4c3C[C@@H]3C(F)(F)[C@]3(C)C4)[nH]c2c1] |
| 26 | 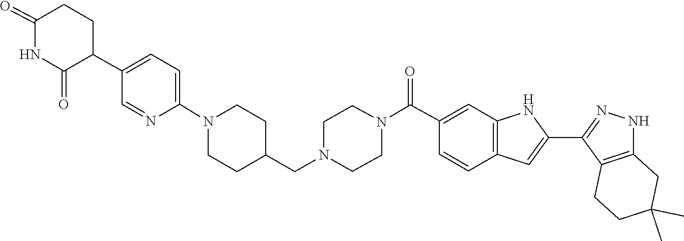 [SMILES: CC1(C)CCc2c(-c3cc4ccc(C(=O)N5CCN(CC6CCN(c7ccc(C8CCC(=O)NC8=O)cn7)CC6)CC5)cc4[nH]3)n[nH]c2C1] |
| 27 | 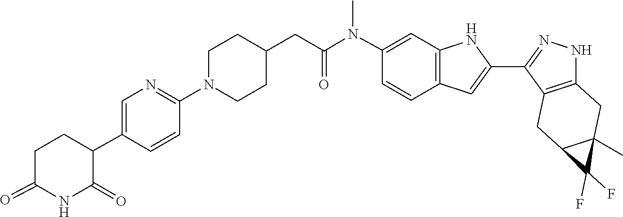 [SMILES: CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1] |
| 28 | 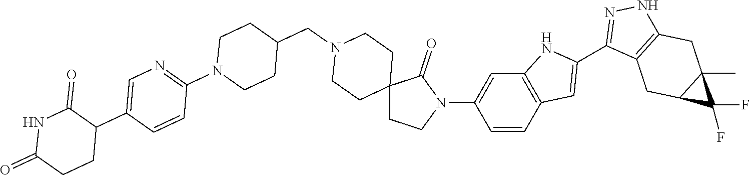 [SMILES: C[C@@]12Cc3[nH]nc(-c4cc5ccc(N6CCC7(CCN(CC8CCN(c9ccc(C%10CCC(=O)NC%10=O)cn9)CC8)CC7)C6=O)cc5[nH]4)c3C[C@@H]1C2(F)F] |
| 30 | 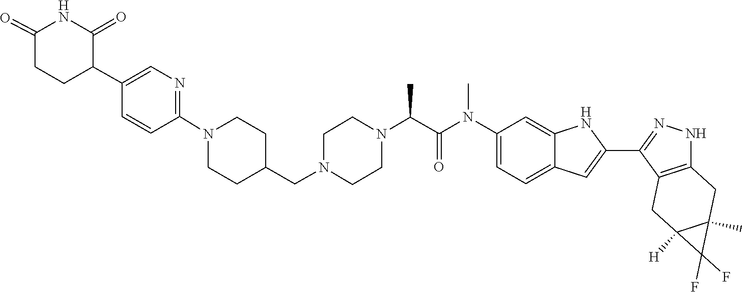 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(C4CCC(=O)NC4=O)cn3)CC2)CC1] |
| 32 | 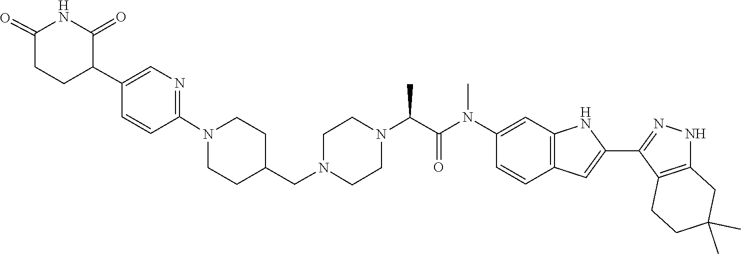 [SMILES: C[C@@H](C(=O)N(C)c1ccc2cc(-c3n[nH]c4c3CCC(C)(C)C4)[nH]c2c1)N1CCN(CC2CCN(c3ccc(C4CCC(=O)NC4=O)cn3)CC2)CC1] |
| 33 | 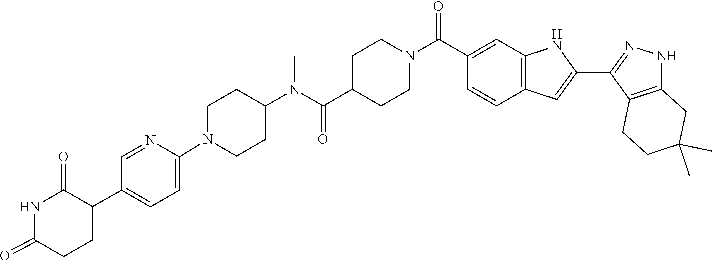 [SMILES: CN(C(=O)C1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1] |
| 34 | 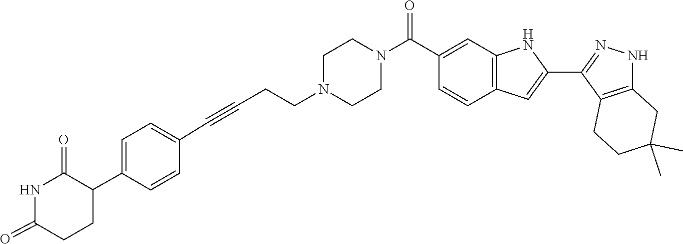 [SMILES: CC1(C)CCc2c(-c3cc4ccc(C(=O)N5CCN(CCC#Cc6ccc(C7CCC(=O)NC7=O)cc6)CC5)cc4[nH]3)n[nH]c2C1] |
| 35 | 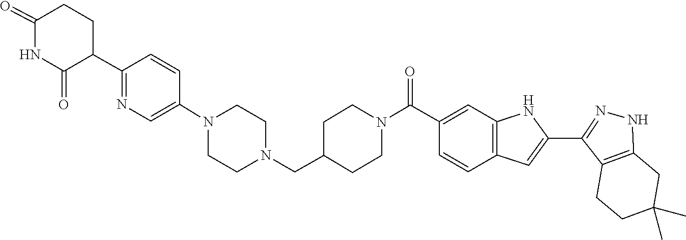 [SMILES: CC1(C)CCc2c(-c3cc4ccc(C(=O)N5CCC(CN6CCN(c7ccc(C8CCC(=O)NC8=O)nc7)CC6)CC5)cc4[nH]3)n[nH]c2C1] |
| 36 | 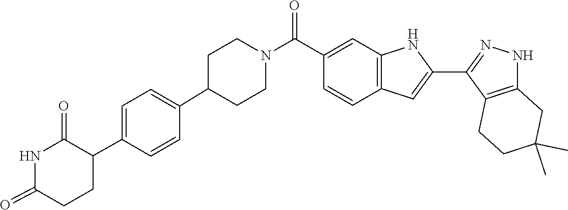 [SMILES: CC1(C)CCc2c(-c3cc4ccc(C(=O)N5CCC(c6ccc(C7CCC(=O)NC7=O)cc6)CC5)cc4[nH]3)n[nH]c2C1] |
| 37 | 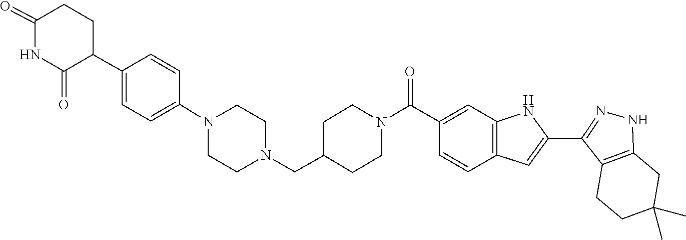 [SMILES: CC1(C)CCc2c(-c3cc4ccc(C(=O)N5CCC(CN6CCN(c7ccc(C8CCC(=O)NC8=O)cc7)CC6)CC5)cc4[nH]3)n[nH]c2C1] |
| 38 | 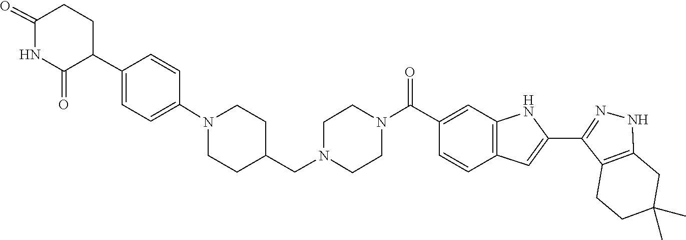 [SMILES: CC1(C)CCc2c(-c3cc4ccc(C(=O)N5CCN(CC6CCN(c7ccc(C8CCC(=O)NC8=O)cc7)CC6)CC5)cc4[nH]3)n[nH]c2C1] |
| 39 | 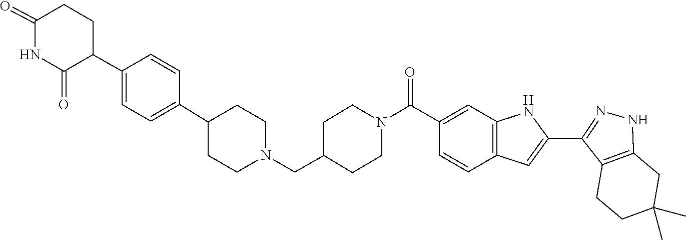 [SMILES: CC1(C)CCc2c(-c3cc4ccc(C(=O)N5CCC(CN6CCC(c7ccc(C8CCC(=O)NC8=O)cc7)CC6)CC5)cc4[nH]3)n[nH]c2C1] |
| 40 | 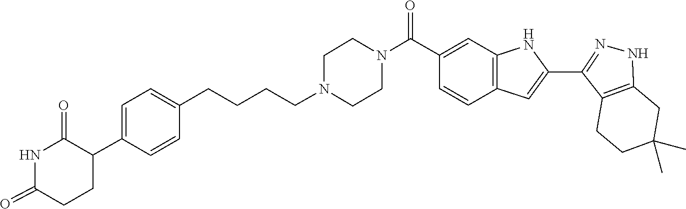 [SMILES: CC1(C)CCc2c(-c3cc4ccc(C(=O)N5CCN(CCCCc6ccc(C7CCC(=O)NC7=O)cc6)CC5)cc4[nH]3)n[nH]c2C1] |
| 41 | 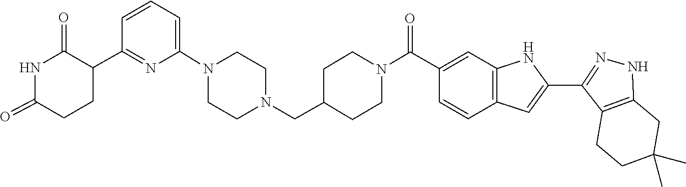 [SMILES: CC1(C)CCc2c(-c3cc4ccc(C(=O)N5CCC(CN6CCN(c7cccc(C8CCC(=O)NC8=O)n7)CC6)CC5)cc4[nH]3)n[nH]c2C1] |
| 42 | 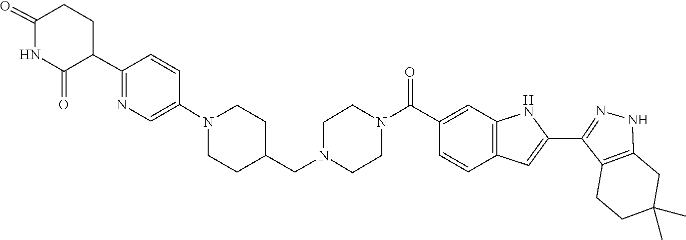 [SMILES: CC1(C)CCc2c(-c3cc4ccc(C(=O)N5CCN(CC6CCN(c7ccc(C8CCC(=O)NC8=O)nc7)CC6)CC5)cc4[nH]3)n[nH]c2C1] |
| 43 | 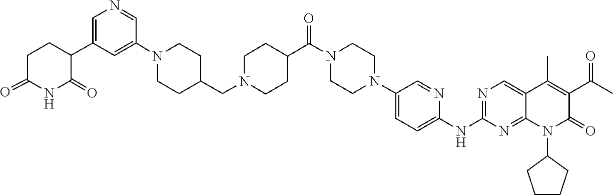 [SMILES: CC(=O)c1c(C)c2cnc(Nc3ccc(N4CCN(C(=O)C5CCN(CC6CCN(c7cncc(C8CCC(=O)NC8=O)c7)CC6)CC5)CC4)cn3)nc2n(C2CCCC2)c1=O] |
| 44 | 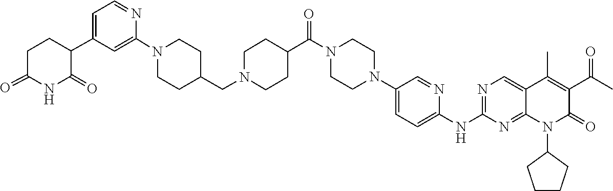 [SMILES: CC(=O)c1c(C)c2cnc(Nc3ccc(N4CCN(C(=O)C5CCN(CC6CCN(c7cc(C8CCC(=O)NC8=O)ccn7)CC6)CC5)CC4)cn3)nc2n(C2CCCC2)c1=O] |
| 45 | 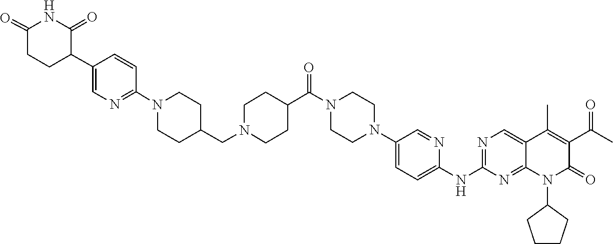 [SMILES: CC(=O)c1c(C)c2cnc(Nc3ccc(N4CCN(C(=O)C5CCN(CC6CCN(c7ccc(C8CCC(=O)NC8=O)cn7)CC6)CC5)CC4)cn3)nc2n(C2CCCC2)c1=O] |
| 46 | 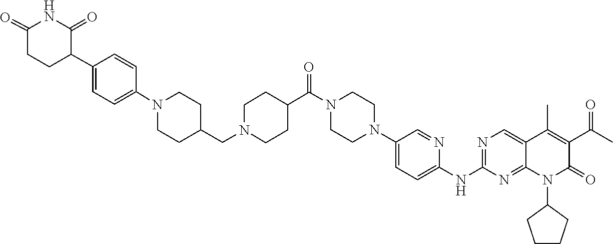 [SMILES: CC(=O)c1c(C)c2cnc(Nc3ccc(N4CCN(C(=O)C5CCN(CC6CCN(c7ccc(C8CCC(=O)NC8=O)cc7)CC6)CC5)CC4)cn3)nc2n(C2CCCC2)c1=O] |
| 47 | 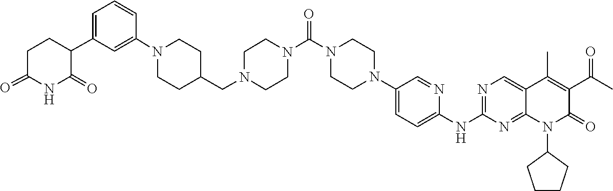 [SMILES: CC(=O)c1c(C)c2cnc(Nc3ccc(N4CCN(C(=O)N5CCN(CC6CCN(c7cccc(C8CCC(=O)NC8=O)c7)CC6)CC5)CC4)cn3)nc2n(C2CCCC2)c1=O] |
| 48 | 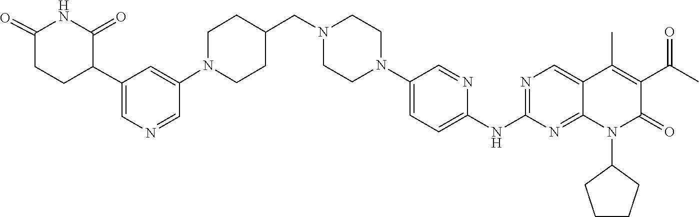 [SMILES: CC(=O)c1c(C)c2cnc(Nc3ccc(N4CCN(CC5CCN(c6cncc(C7CCC(=O)NC7=O)c6)CC5)CC4)cn3)nc2n(C2CCCC2)c1=O] |
| 49 | 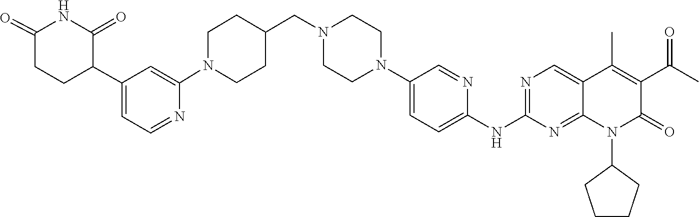 [SMILES: CC(=O)c1c(C)c2cnc(Nc3ccc(N4CCN(CC5CCN(c6cc(C7CCC(=O)NC7=O)ccn6)CC5)CC4)cn3)nc2n(C2CCCC2)c1=O] |
| 50 | 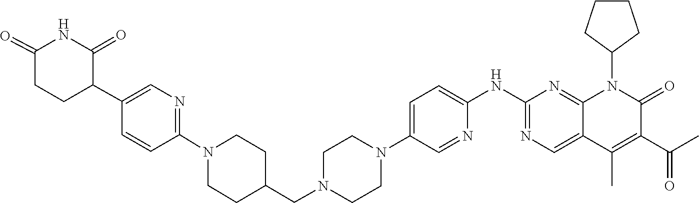 [SMILES: CC(=O)c1c(C)c2cnc(Nc3ccc(N4CCN(CC5CCN(c6ccc(C7CCC(=O)NC7=O)cn6)CC5)CC4)cn3)nc2n(C2CCCC2)c1=O] |
| 51 | 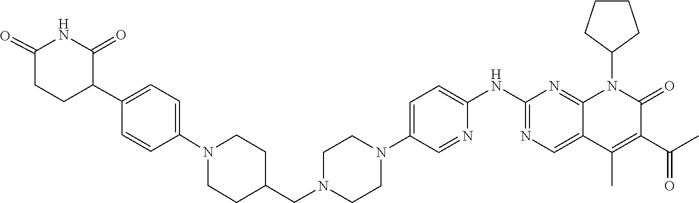 [SMILES: CC(=O)c1c(C)c2cnc(Nc3ccc(N4CCN(CC5CCN(c6ccc(C7CCC(=O)NC7=O)cc6)CC5)CC4)cn3)nc2n(C2CCCC2)c1=O] |
| 52 | 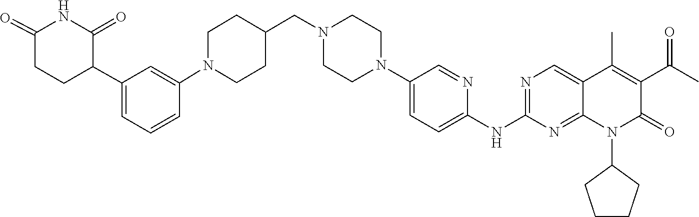 [SMILES: CC(=O)c1c(C)c2cnc(Nc3ccc(N4CCN(CC5CCN(c6cccc(C7CCC(=O)NC7=O)c6)CC5)CC4)cn3)nc2n(C2CCCC2)c1=O] |
| 53 | 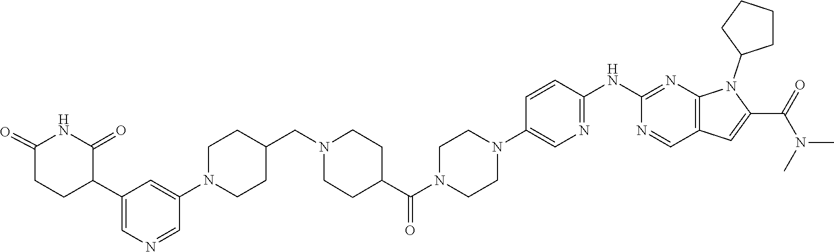 [SMILES: CN(C)C(=O)c1cc2cnc(Nc3ccc(N4CCN(C(=O)C5CCN(CC6CCN(c7cncc(C8CCC(=O)NC8=O)c7)CC6)CC5)CC4)cn3)nc2n1C1CCCC1] |
| 54 | 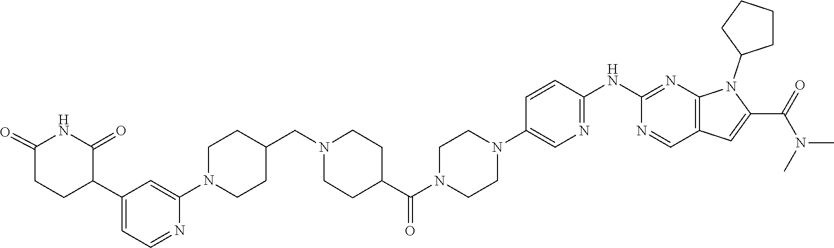 [SMILES: CN(C)C(=O)c1cc2cnc(Nc3ccc(N4CCN(C(=O)C5CCN(CC6CCN(c7cc(C8CCC(=O)NC8=O)ccn7)CC6)CC5)CC4)cn3)nc2n1C1CCCC1] |
| 55 | 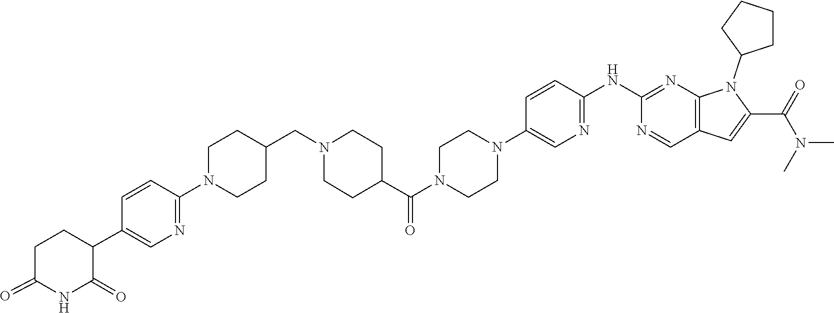 [SMILES: CN(C)C(=O)c1cc2cnc(Nc3ccc(N4CCN(C(=O)C5CCN(CC6CCN(c7ccc(C8CCC(=O)NC8=O)cn7)CC6)CC5)CC4)cn3)nc2n1C1CCCC1] |
| 56 | 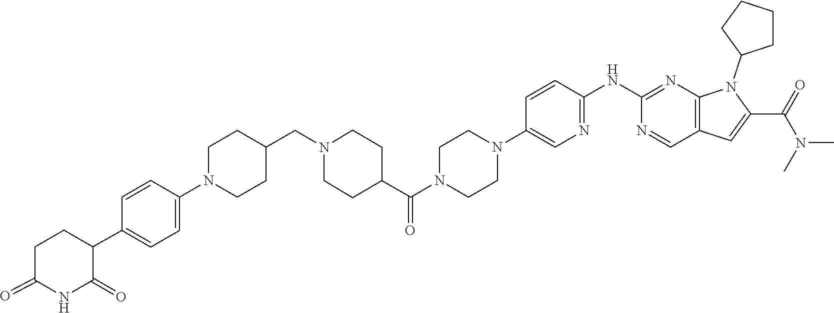 [SMILES: CN(C)C(=O)c1cc2cnc(Nc3ccc(N4CCN(C(=O)C5CCN(CC6CCN(c7ccc(C8CCC(=O)NC8=O)cc7)CC6)CC5)CC4)cn3)nc2n1C1CCCC1] |
| 57 | 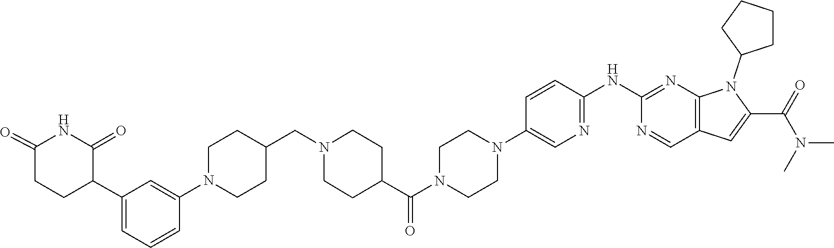 [SMILES: CN(C)C(=O)c1cc2cnc(Nc3ccc(N4CCN(C(=O)C5CCN(CC6CCN(c7cccc(C8CCC(=O)NC8=O)c7)CC6)CC5)CC4)cn3)nc2n1C1CCCC1] |
| 58 | 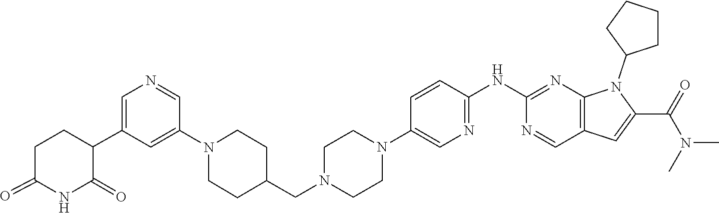 [SMILES: CN(C)C(=O)c1cc2cnc(Nc3ccc(N4CCN(CC5CCN(c6cncc(C7CCC(=O)NC7=O)c6)CC5)CC4)cn3)nc2n1C1CCCC1] |
| 59 | 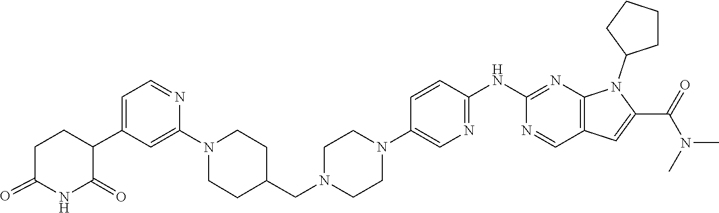 [SMILES: CN(C)C(=O)c1cc2cnc(Nc3ccc(N4CCN(CC5CCN(c6cc(C7CCC(=O)NC7=O)ccn6)CC5)CC4)cn3)nc2n1C1CCCC1] |
| 60 | 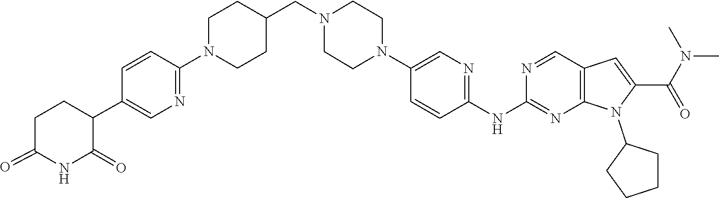 [SMILES: CN(C)C(=O)c1cc2cnc(Nc3ccc(N4CCN(CC5CCN(c6ccc(C7CCC(=O)NC7=O)cn6)CC5)CC4)cn3)nc2n1C1CCCC1] |
| 61 | 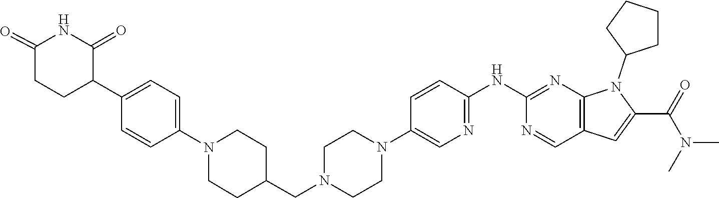 [SMILES: CN(C)C(=O)c1cc2cnc(Nc3ccc(N4CCN(CC5CCN(c6ccc(C7CCC(=O)NC7=O)cc6)CC5)CC4)cn3)nc2n1C1CCCC1] |
| 62 | 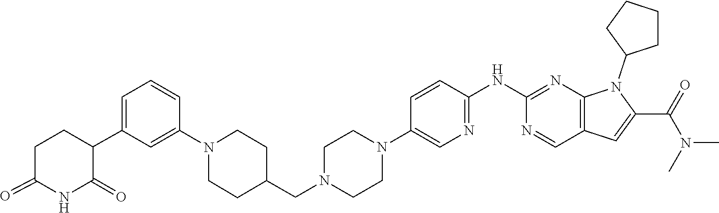 [SMILES: CN(C)C(=O)c1cc2cnc(Nc3ccc(N4CCN(CC5CCN(c6cccc(C7CCC(=O)NC7=O)c6)CC5)CC4)cn3)nc2n1C1CCCC1] |
| 63 | 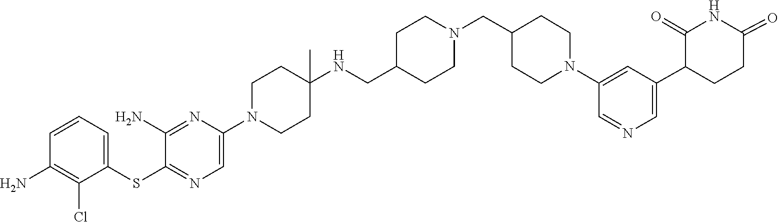 [SMILES: CC1(NCC2CCN(CC3CCN(c4cncc(C5CCC(=O)NC5=O)c4)CC3)CC2)CCN(c2cnc(Sc3cccc(N)c3Cl)c(N)n2)CC1] |
| 64 | 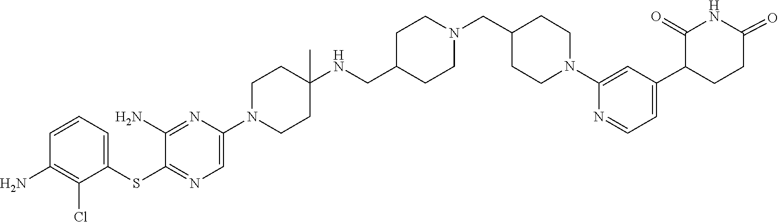 [SMILES: CC1(NCC2CCN(CC3CCN(c4cc(C5CCC(=O)NC5=O)ccn4)CC3)CC2)CCN(c2cnc(Sc3cccc(N)c3Cl)c(N)n2)CC1] |
| 65 | 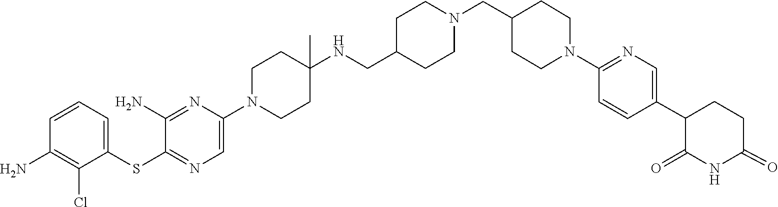 [SMILES: CC1(NCC2CCN(CC3CCN(c4ccc(C5CCC(=O)NC5=O)cn4)CC3)CC2)CCN(c2cnc(Sc3cccc(N)c3Cl)c(N)n2)CC1] |
| 66 | 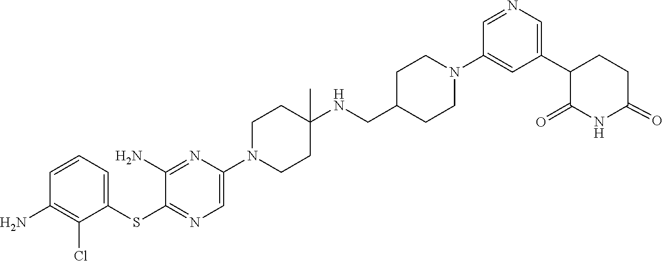 [SMILES: CC1(NCC2CCN(c3cncc(C4CCC(=O)NC4=O)c3)CC2)CCN(c2cnc(Sc3cccc(N)c3Cl)c(N)n2)CC1] |
| 67 | 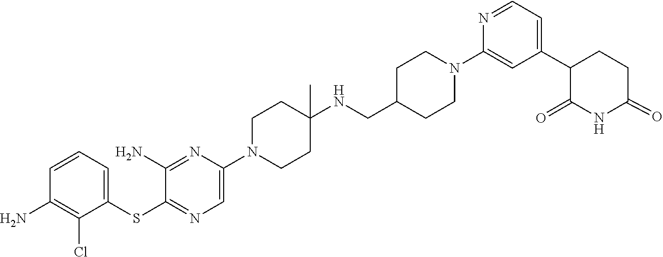 [SMILES: CC1(NCC2CCN(c3cc(C4CCC(=O)NC4=O)ccn3)CC2)CCN(c2cnc(Sc3cccc(N)c3Cl)c(N)n2)CC1] |
| 68 | 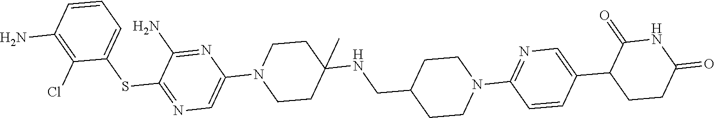 [SMILES: CC1(NCC2CCN(c3ccc(C4CCC(=O)NC4=O)cn3)CC2)CCN(c2cnc(Sc3cccc(N)c3Cl)c(N)n2)CC1] |
| 69 | 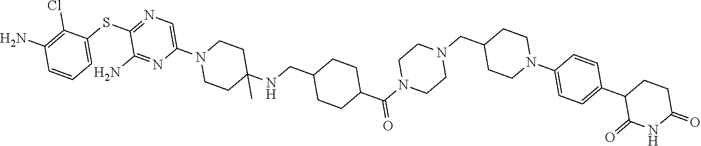 [SMILES: CC1(NCC2CCC(C(=O)N3CCN(CC4CCN(c5ccc(C6CCC(=O)NC6=O)cc5)CC4)CC3)CC2)CCN(c2cnc(Sc3cccc(N)c3Cl)c(N)n2)CC1] |
| 70 | 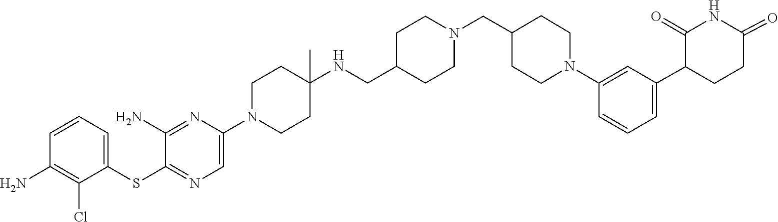 [SMILES: CC1(NCC2CCN(CC3CCN(c4cccc(C5CCC(=O)NC5=O)c4)CC3)CC2)CCN(c2cnc(Sc3cccc(N)c3Cl)c(N)n2)CC1] |
| 71 | 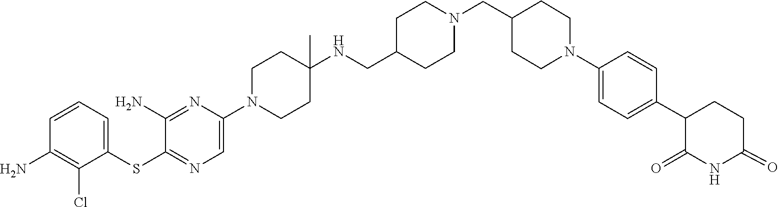 [SMILES: CC1(NCC2CCN(CC3CCN(c4ccc(C5CCC(=O)NC5=O)cc4)CC3)CC2)CCN(c2cnc(Sc3cccc(N)c3Cl)c(N)n2)CC1] |
| 72 | 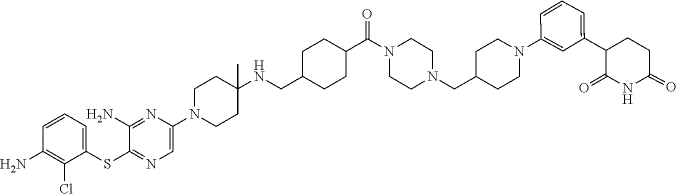 [SMILES: CC1(NCC2CCC(C(=O)N3CCN(CC4CCN(c5cccc(C6CCC(=O)NC6=O)c5)CC4)CC3)CC2)CCN(c2cnc(Sc3cccc(N)c3Cl)c(N)n2)CC1] |
| 73 | 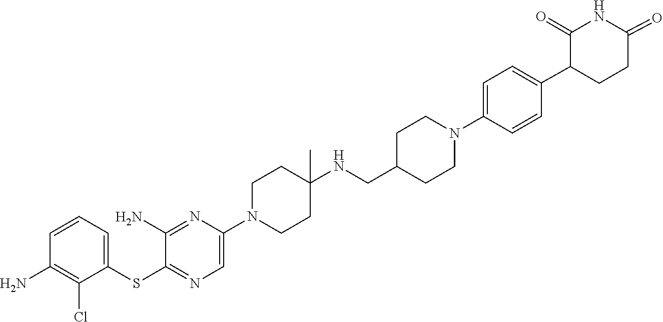 [SMILES: CC1(NCC2CCN(c3ccc(C4CCC(=O)NC4=O)cc3)CC2)CCN(c2cnc(Sc3cccc(N)c3Cl)c(N)n2)CC1] |
| 74 | 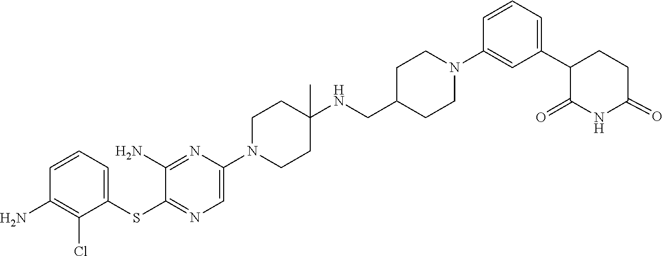 [SMILES: CC1(NCC2CCN(c3cccc(C4CCC(=O)NC4=O)c3)CC2)CCN(c2cnc(Sc3cccc(N)c3Cl)c(N)n2)CC1] |
| 75 | 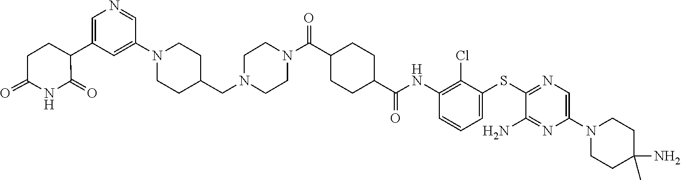 [SMILES: CC1(N)CCN(c2cnc(Sc3cccc(NC(=O)C4CCC(C(=O)N5CCN(CC6CCN(c7cncc(C8CCC(=O)NC8=O)c7)CC6)CC5)CC4)c3Cl)c(N)n2)CC1] |
| 76 | 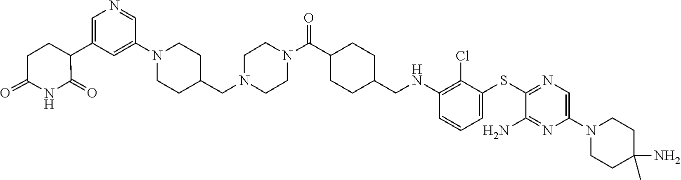 [SMILES: CC1(N)CCN(c2cnc(Sc3cccc(NCC4CCC(C(=O)N5CCN(CC6CCN(c7cncc(C8CCC(=O)NC8=O)c7)CC6)CC5)CC4)c3Cl)c(N)n2)CC1] |
| 77 | 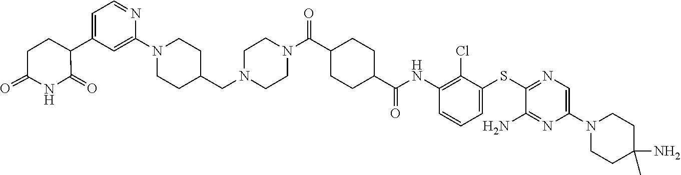 [SMILES: CC1(N)CCN(c2cnc(Sc3cccc(NC(=O)C4CCC(C(=O)N5CCN(CC6CCN(c7cc(C8CCC(=O)NC8=O)ccn7)CC6)CC5)CC4)c3Cl)c(N)n2)CC1] |
| 78 | 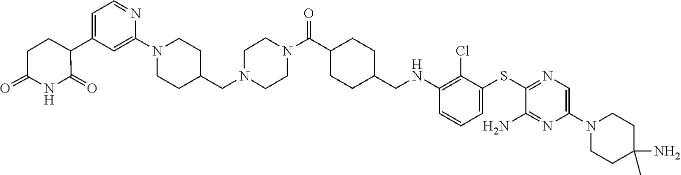 [SMILES: CC1(N)CCN(c2cnc(Sc3cccc(NCC4CCC(C(=O)N5CCN(CC6CCN(c7cc(C8CCC(=O)NC8=O)ccn7)CC6)CC5)CC4)c3Cl)c(N)n2)CC1] |
| 79 | 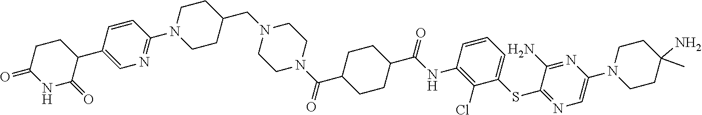 [SMILES: CC1(N)CCN(c2cnc(Sc3cccc(NC(=O)C4CCC(C(=O)N5CCN(CC6CCN(c7ccc(C8CCC(=O)NC8=O)cn7)CC6)CC5)CC4)c3Cl)c(N)n2)CC1] |
| 80 | 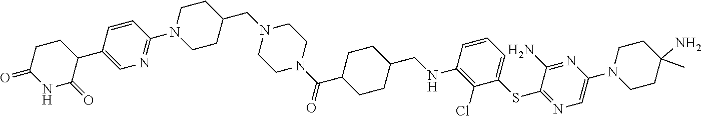 [SMILES: CC1(N)CCN(c2cnc(Sc3cccc(NCC4CCC(C(=O)N5CCN(CC6CCN(c7ccc(C8CCC(=O)NC8=O)cn7)CC6)CC5)CC4)c3Cl)c(N)n2)CC1] |
| 81 | 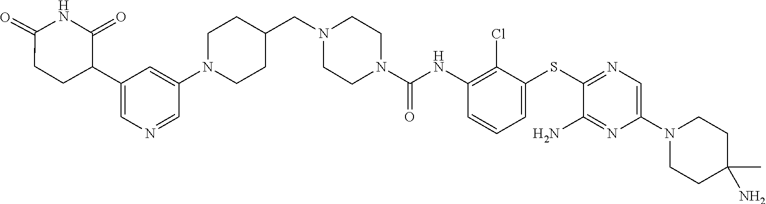 [SMILES: CC1(N)CCN(c2cnc(Sc3cccc(NC(=O)N4CCN(CC5CCN(c6cncc(C7CCC(=O)NC7=O)c6)CC5)CC4)c3Cl)c(N)n2)CC1] |
| 82 | 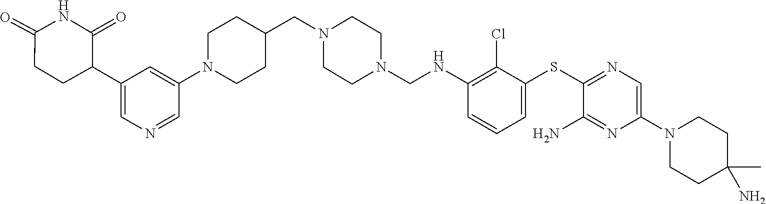 [SMILES: CC1(N)CCN(c2cnc(Sc3cccc(NCN4CCN(CC5CCN(c6cncc(C7CCC(=O)NC7=O)c6)CC5)CC4)c3Cl)c(N)n2)CC1] |
| 83 | 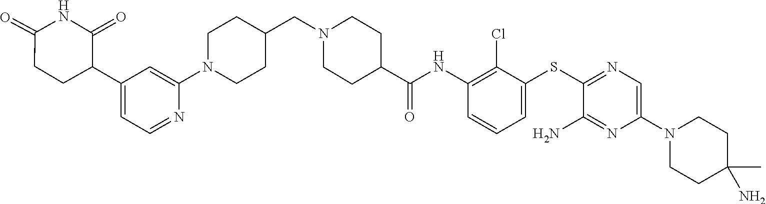 [SMILES: CC1(N)CCN(c2cnc(Sc3cccc(NC(=O)C4CCN(CC5CCN(c6cc(C7CCC(=O)NC7=O)ccn6)CC5)CC4)c3Cl)c(N)n2)CC1] |
| 84 | 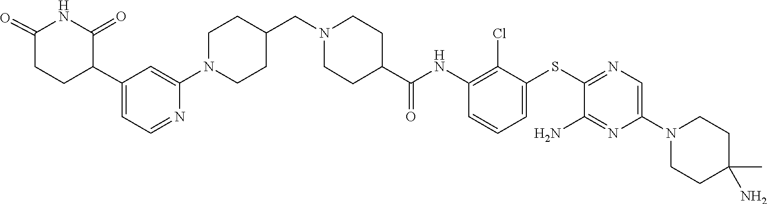 [SMILES: CC1(N)CCN(c2cnc(Sc3cccc(NC(=O)C4CCN(CC5CCN(c6cc(C7CCC(=O)NC7=O)ccn6)CC5)CC4)c3Cl)c(N)n2)CC1] |
| 85 | 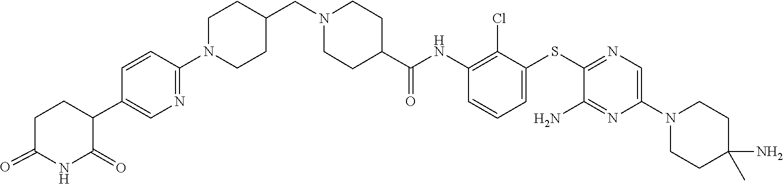 [SMILES: CC1(N)CCN(c2cnc(Sc3cccc(NC(=O)C4CCN(CC5CCN(c6ccc(C7CCC(=O)NC7=O)cn6)CC5)CC4)c3Cl)c(N)n2)CC1] |
| 86 | 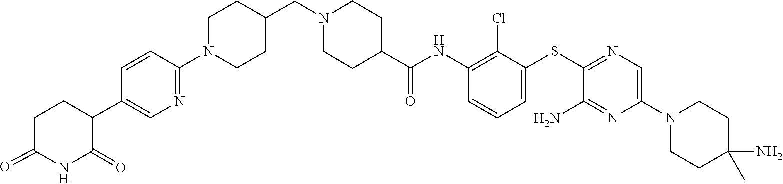 [SMILES: CC1(N)CCN(c2cnc(Sc3cccc(NC(=O)C4CCN(CC5CCN(c6ccc(C7CCC(=O)NC7=O)cn6)CC5)CC4)c3Cl)c(N)n2)CC1] |
| 87 | 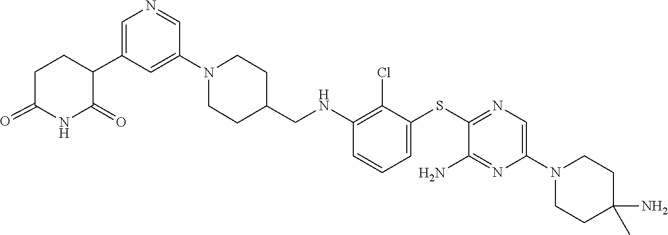 [SMILES: CC1(N)CCN(c2cnc(Sc3cccc(NCC4CCN(c5cncc(C6CCC(=O)NC6=O)c5)CC4)c3Cl)c(N)n2)CC1] |
| 88 | 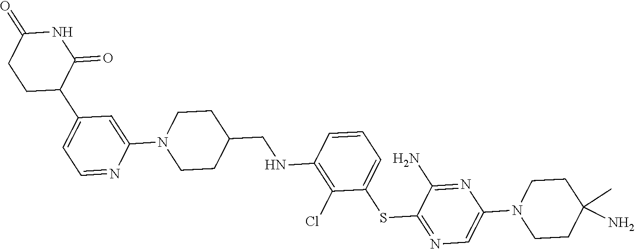 [SMILES: CC1(N)CCN(c2cnc(Sc3cccc(NCC4CCN(c5cc(C6CCC(=O)NC6=O)ccn5)CC4)c3Cl)c(N)n2)CC1] |
| 89 | 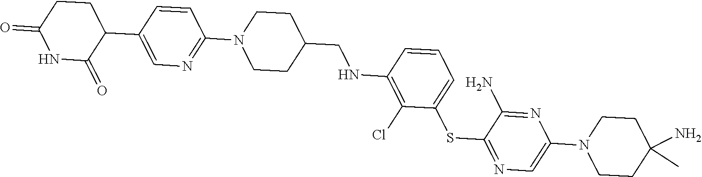 [SMILES: CC1(N)CCN(c2cnc(Sc3cccc(NCC4CCN(c5ccc(C6CCC(=O)NC6=O)cn5)CC4)c3Cl)c(N)n2)CC1] |
| 90 | 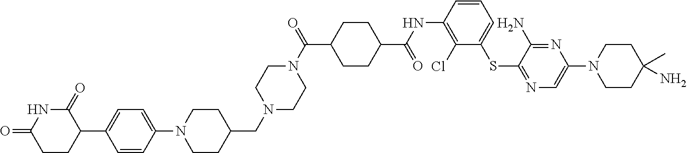 [SMILES: CC1(N)CCN(c2cnc(Sc3cccc(NC(=O)C4CCC(C(=O)N5CCN(CC6CCN(c7ccc(C8CCC(=O)NC8=O)cc7)CC6)CC5)CC4)c3Cl)c(N)n2)CC1] |
| 91 | 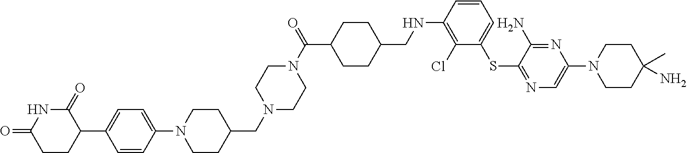 [SMILES: CC1(N)CCN(c2cnc(Sc3cccc(NCC4CCC(C(=O)N5CCN(CC6CCN(c7ccc(C8CCC(=O)NC8=O)cc7)CC6)CC5)CC4)c3Cl)c(N)n2)CC1] |
| 92 | 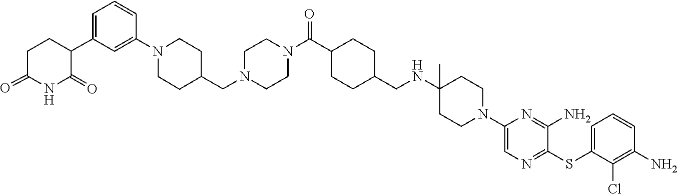 [SMILES: CC1(NCC2CCC(C(=O)N3CCN(CC4CCN(c5cccc(C6CCC(=O)NC6=O)c5)CC4)CC3)CC2)CCN(c2cnc(Sc3cccc(N)c3Cl)c(N)n2)CC1] |
| 93 | 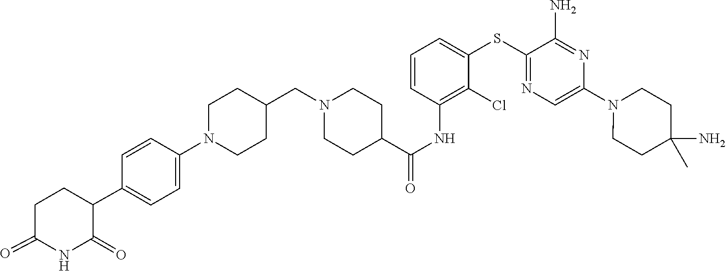 [SMILES: CC1(N)CCN(c2cnc(Sc3cccc(NC(=O)C4CCN(CC5CCN(c6ccc(C7CCC(=O)NC7=O)cc6)CC5)CC4)c3Cl)c(N)n2)CC1] |
| 94 | 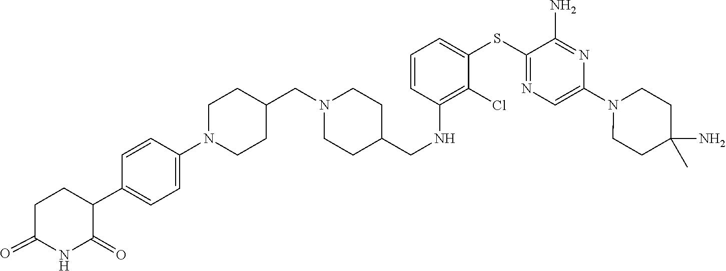 [SMILES: CC1(N)CCN(c2cnc(Sc3cccc(NCC4CCN(CC5CCN(c6ccc(C7CCC(=O)NC7=O)cc6)CC5)CC4)c3Cl)c(N)n2)CC1] |
| 95 | 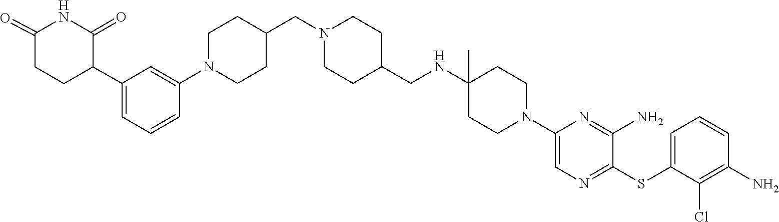 [SMILES: CC1(NCC2CCN(CC3CCN(c4cccc(C5CCC(=O)NC5=O)c4)CC3)CC2)CCN(c2cnc(Sc3cccc(N)c3Cl)c(N)n2)CC1] |
| 96 | 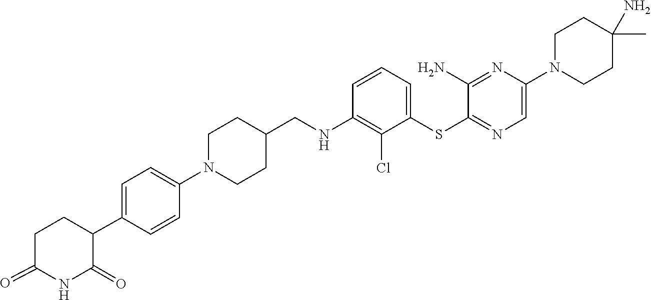 [SMILES: CC1(N)CCN(c2cnc(Sc3cccc(NCC4CCN(c5ccc(C6CCC(=O)NC6=O)cc5)CC4)c3Cl)c(N)n2)CC1] |
| 97 | 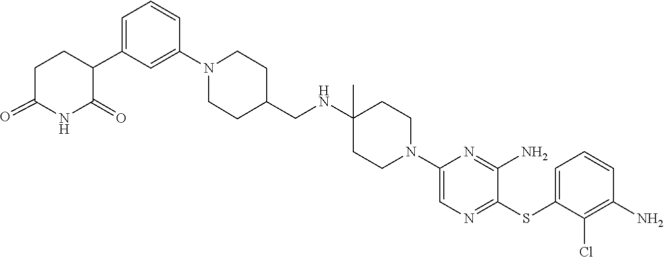 [SMILES: CC1(NCC2CCN(c3cccc(C4CCC(=O)NC4=O)c3)CC2)CCN(c2cnc(Sc3cccc(N)c3Cl)c(N)n2)CC1] |
| 98 | 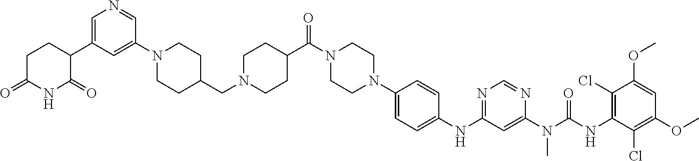 [SMILES: COc1cc(OC)c(Cl)c(NC(=O)N(C)c2cc(Nc3ccc(N4CCN(C(=O)C5CCN(CC6CCN(c7cncc(C8CCC(=O)NC8=O)c7)CC6)CC5)CC4)cc3)ncn2)c1Cl] |
| 99 | 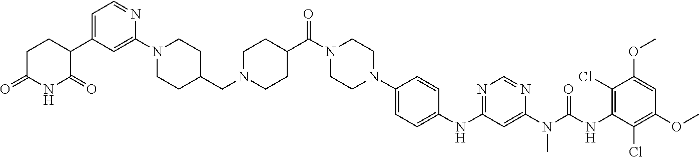 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] |
| 100 | 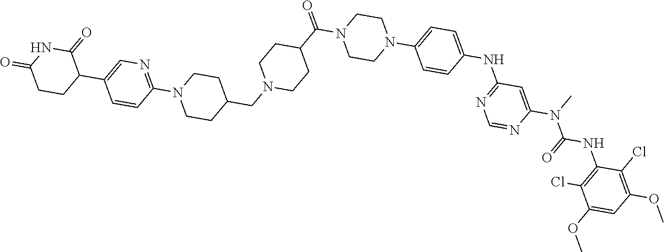 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cn7)CC6)CC5)CC4)cc3)ncn2)c1Cl] |
| 101 | 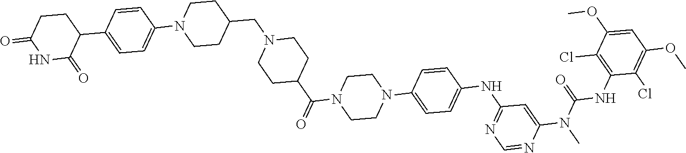 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cc7)CC6)CC5)CC4)cc3)ncn2)c1Cl] |
| 102 | 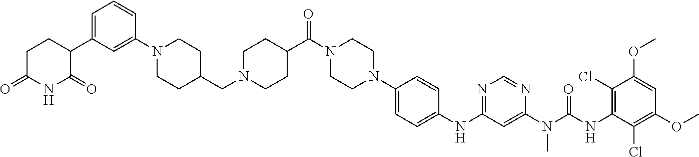 [SMILES: COc1cc(OC)c(Cl)c(NC(=O)N(C)c2cc(Nc3ccc(N4CCN(C(=O)C5CCN(CC6CCN(c7cccc(C8CCC(=O)NC8=O)c7)CC6)CC5)CC4)cc3)ncn2)c1Cl] |
| 103 | 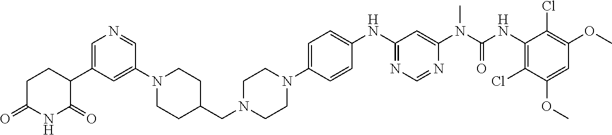 [SMILES: COc1cc(OC)c(Cl)c(NC(=O)N(C)c2cc(Nc3ccc(N4CCN(CC5CCN(c6cncc(C7CCC(=O)NC7=O)c6)CC5)CC4)cc3)ncn2)c1Cl] |
| 104 | 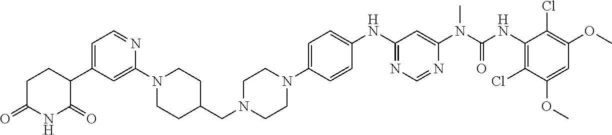 [SMILES: COc1cc(OC)c(Cl)c(NC(=O)N(C)c2cc(Nc3ccc(N4CCN(CC5CCN(c6cc(C7CCC(=O)NC7=O)ccn6)CC5)CC4)cc3)ncn2)c1Cl] |
| 105 | 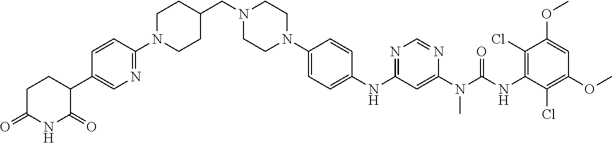 [SMILES: COc1cc(OC)c(Cl)c(NC(=O)N(C)c2cc(Nc3ccc(N4CCN(CC5CCN(c6ccc(C7CCC(=O)NC7=O)cn6)CC5)CC4)cc3)ncn2)c1Cl] |
| 106 | 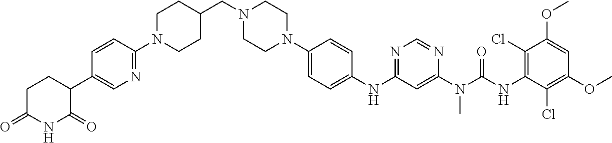 [SMILES: COc1cc(OC)c(Cl)c(NC(=O)N(C)c2cc(Nc3ccc(N4CCN(CC5CCN(c6ccc(C7CCC(=O)NC7=O)cn6)CC5)CC4)cc3)ncn2)c1Cl] |
| 107 | 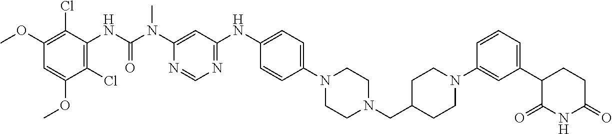 [SMILES: COc1cc(OC)c(Cl)c(NC(=O)N(C)c2cc(Nc3ccc(N4CCN(CC5CCN(c6cccc(C7CCC(=O)NC7=O)c6)CC5)CC4)cc3)ncn2)c1Cl] |
| 108 | 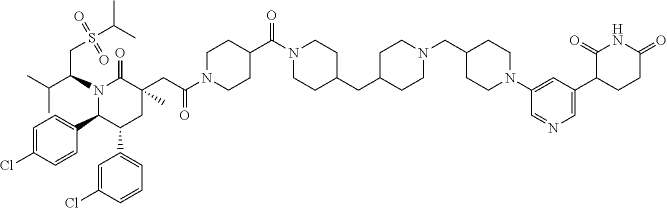 [SMILES: CC(C)[C@@H](CS(=O)(=O)C(C)C)N1C(=O)[C@@](C)(CC(=O)N2CCC(C(=O)N3CCC(CC4CCN(CC5CCN(c6cncc(C7CCC(=O)NC7=O)c6)CC5)CC4)CC3)CC2)C[C@H](c2cccc(Cl)c2)[C@H]1c1ccc(Cl)cc1] |
| 109 | 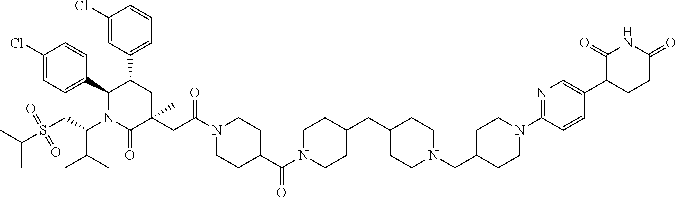 [SMILES: CC(C)[C@H](CS(=O)(=O)C(C)C)N1C(=O)[C@](C)(CC(=O)N2CCC(C(=O)N3CCC(CC4CCN(CC5CCN(c6ccc(C7CCC(=O)NC7=O)cn6)CC5)CC4)CC3)CC2)C[C@@H](c2cccc(Cl)c2)[C@@H]1c1ccc(Cl)cc1] |
| 110 | 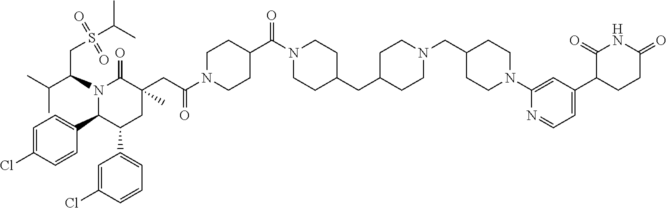 [SMILES: CC(C)[C@@H](CS(=O)(=O)C(C)C)N1C(=O)[C@@](C)(CC(=O)N2CCC(C(=O)N3CCC(CC4CCN(CC5CCN(c6cc(C7CCC(=O)NC7=O)ccn6)CC5)CC4)CC3)CC2)C[C@H](c2cccc(Cl)c2)[C@H]1c1ccc(Cl)cc1] |
| 111 | 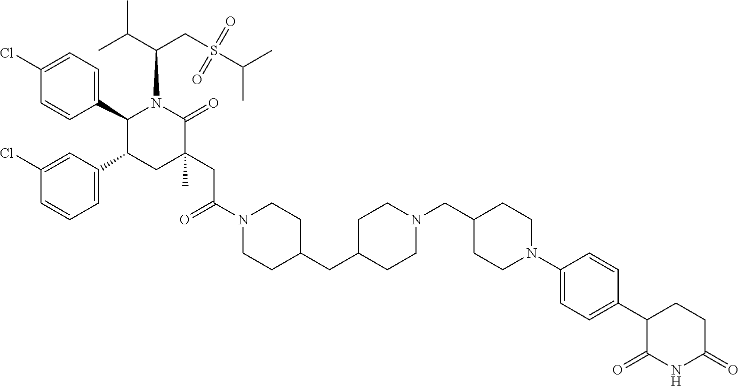 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cc5)CC4)CC3)CC2)C[C@H](c2cccc(Cl)c2)[C@H]1c1ccc(Cl)cc1] |
| 112 | 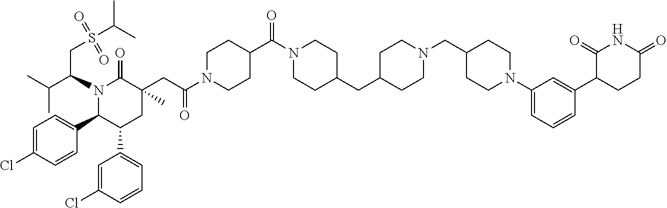 [SMILES: CC(C)[C@@H](CS(=O)(=O)C(C)C)N1C(=O)[C@@](C)(CC(=O)N2CCC(C(=O)N3CCC(CC4CCN(CC5CCN(c6cccc(C7CCC(=O)NC7=O)c6)CC5)CC4)CC3)CC2)C[C@H](c2cccc(Cl)c2)[C@H]1c1ccc(Cl)cc1] |
| 113 | 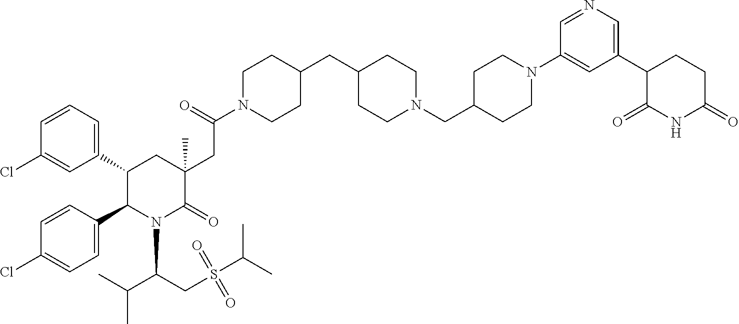 [SMILES: CC(C)[C@H](CS(=O)(=O)C(C)C)N1C(=O)[C@](C)(CC(=O)N2CCC(CC3CCN(CC4CCN(c5cncc(C6CCC(=O)NC6=O)c5)CC4)CC3)CC2)C[C@@H](c2cccc(Cl)c2)[C@@H]1c1ccc(Cl)cc1] |
| 114 | 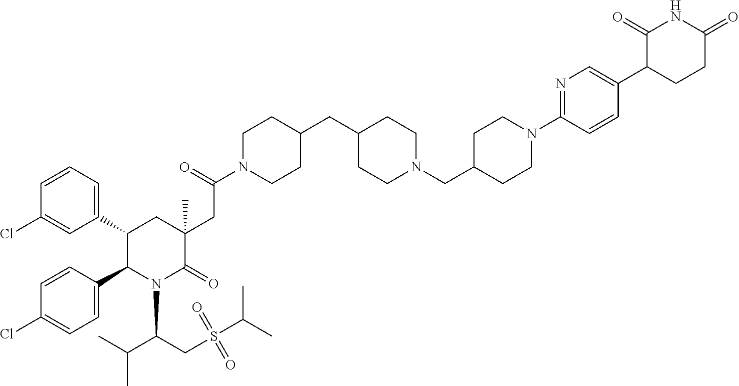 [SMILES: CC(C)[C@H](CS(=O)(=O)C(C)C)N1C(=O)[C@](C)(CC(=O)N2CCC(CC3CCN(CC4CCN(c5ccc(C6CCC(=O)NC6=O)cn5)CC4)CC3)CC2)C[C@@H](c2cccc(Cl)c2)[C@@H]1c1ccc(Cl)cc1] |
| 115 | 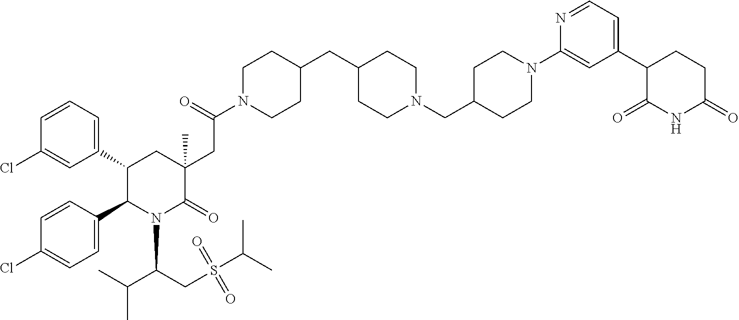 [SMILES: CC(C)[C@H](CS(=O)(=O)C(C)C)N1C(=O)[C@](C)(CC(=O)N2CCC(CC3CCN(CC4CCN(c5cc(C6CCC(=O)NC6=O)ccn5)CC4)CC3)CC2)C[C@@H](c2cccc(Cl)c2)[C@@H]1c1ccc(Cl)cc1] |
| 116 | 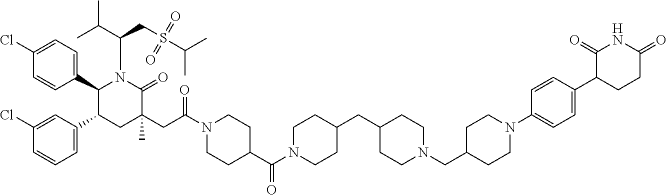 [SMILES: CC(C)[C@@H](CS(=O)(=O)C(C)C)N1C(=O)[C@@](C)(CC(=O)N2CCC(C(=O)N3CCC(CC4CCN(CC5CCN(c6ccc(C7CCC(=O)NC7=O)cc6)CC5)CC4)CC3)CC2)C[C@H](c2cccc(Cl)c2)[C@H]1c1ccc(Cl)cc1] |
| 117 | 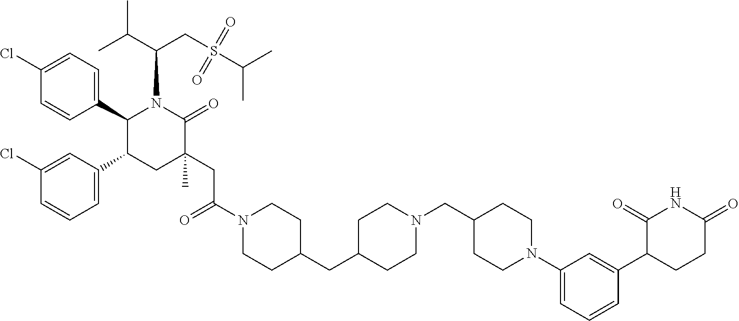 [SMILES: CC(C)[C@@H](CS(=O)(=O)C(C)C)N1C(=O)[C@@](C)(CC(=O)N2CCC(CC3CCN(CC4CCN(c5cccc(C6CCC(=O)NC6=O)c5)CC4)CC3)CC2)C[C@H](c2cccc(Cl)c2)[C@H]1c1ccc(Cl)cc1] |
| 118 | 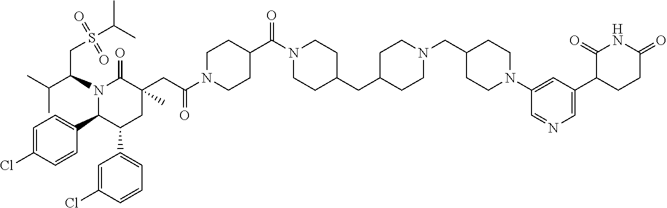 [SMILES: CC(C)[C@@H](CS(=O)(=O)C(C)C)N1C(=O)[C@@](C)(CC(=O)N2CCC(C(=O)N3CCC(CC4CCN(CC5CCN(c6cncc(C7CCC(=O)NC7=O)c6)CC5)CC4)CC3)CC2)C[C@H](c2cccc(Cl)c2)[C@H]1c1ccc(Cl)cc1] |
| 119 | 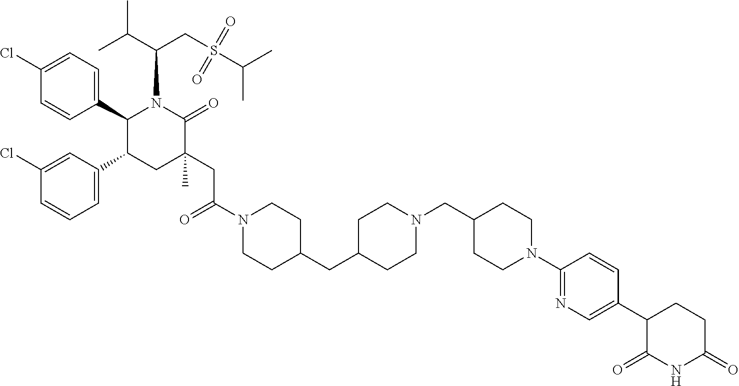 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cn5)CC4)CC3)CC2)C[C@H](c2cccc(Cl)c2)[C@H]1c1ccc(Cl)cc1] |
| 120 | 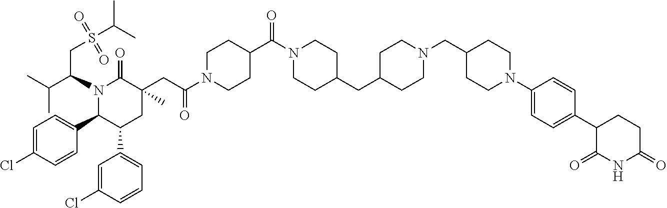 [SMILES: CC(C)[C@@H](CS(=O)(=O)C(C)C)N1C(=O)[C@@](C)(CC(=O)N2CCC(C(=O)N3CCC(CC4CCN(CC5CCN(c6ccc(C7CCC(=O)NC7=O)cc6)CC5)CC4)CC3)CC2)C[C@H](c2cccc(Cl)c2)[C@H]1c1ccc(Cl)cc1] |
| 121 | 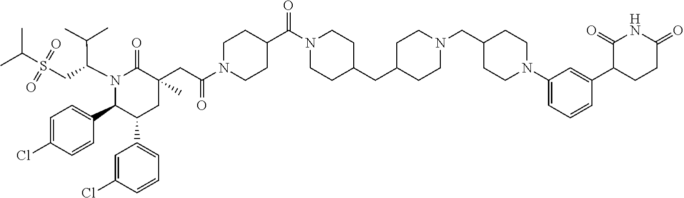 [SMILES: CC(C)[C@@H](CS(=O)(=O)C(C)C)N1C(=O)[C@@](C)(CC(=O)N2CCC(C(=O)N3CCC(CC4CCN(CC5CCN(c6cccc(C7CCC(=O)NC7=O)c6)CC5)CC4)CC3)CC2)C[C@H](c2cccc(Cl)c2)[C@H]1c1ccc(Cl)cc1] |
| 122 | 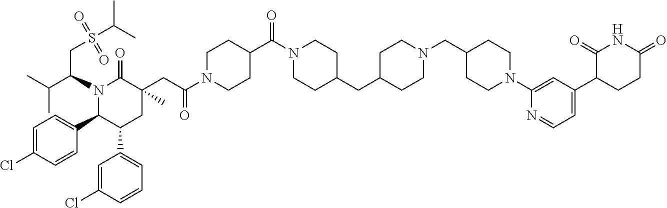 [SMILES: CC(C)[C@@H](CS(=O)(=O)C(C)C)N1C(=O)[C@@](C)(CC(=O)N2CCC(C(=O)N3CCC(CC4CCN(CC5CCN(c6cc(C7CCC(=O)NC7=O)ccn6)CC5)CC4)CC3)CC2)C[C@H](c2cccc(Cl)c2)[C@H]1c1ccc(Cl)cc1] |
| 123 | 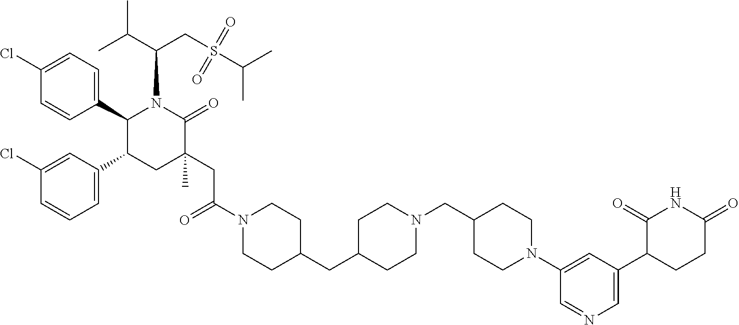 [SMILES: CC(C)[C@@H](CS(=O)(=O)C(C)C)N1C(=O)[C@@](C)(CC(=O)N2CCC(CC3CCN(CC4CCN(c5cncc(C6CCC(=O)NC6=O)c5)CC4)CC3)CC2)C[C@H](c2cccc(Cl)c2)[C@H]1c1ccc(Cl)cc1] |
| 124 | 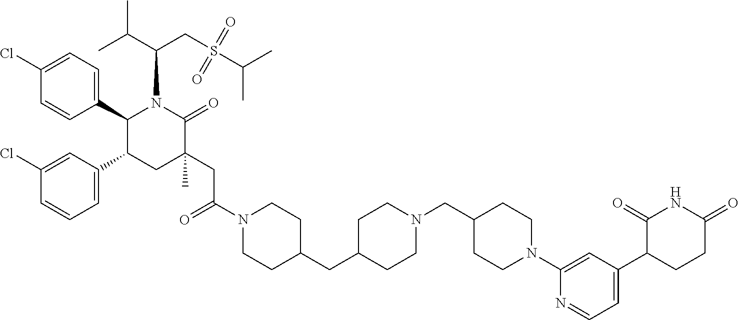 [SMILES: CC(C)[C@@H](CS(=O)(=O)C(C)C)N1C(=O)[C@@](C)(CC(=O)N2CCC(CC3CCN(CC4CCN(c5cc(C6CCC(=O)NC6=O)ccn5)CC4)CC3)CC2)C[C@H](c2cccc(Cl)c2)[C@H]1c1ccc(Cl)cc1] |
| 125 | 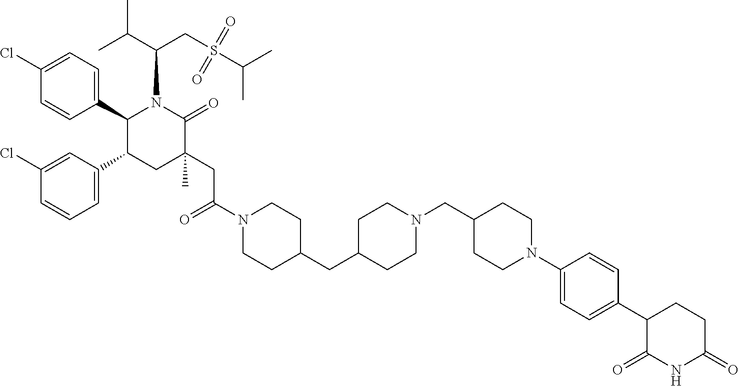 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cc5)CC4)CC3)CC2)C[C@H](c2cccc(Cl)c2)[C@H]1c1ccc(Cl)cc1] |
| 126 | 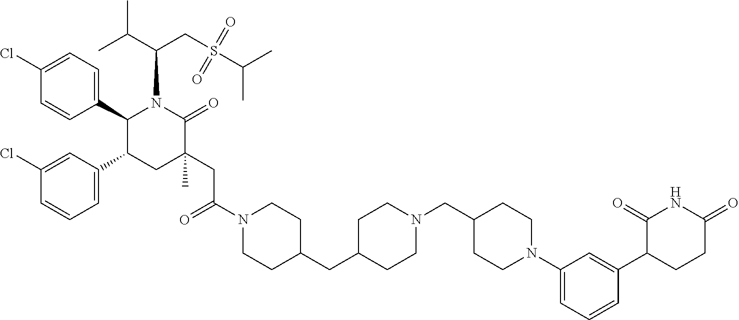 [SMILES: CC(C)[C@@H](CS(=O)(=O)C(C)C)N1C(=O)[C@@](C)(CC(=O)N2CCC(CC3CCN(CC4CCN(c5cccc(C6CCC(=O)NC6=O)c5)CC4)CC3)CC2)C[C@H](c2cccc(Cl)c2)[C@H]1c1ccc(Cl)cc1] |
| 127 | 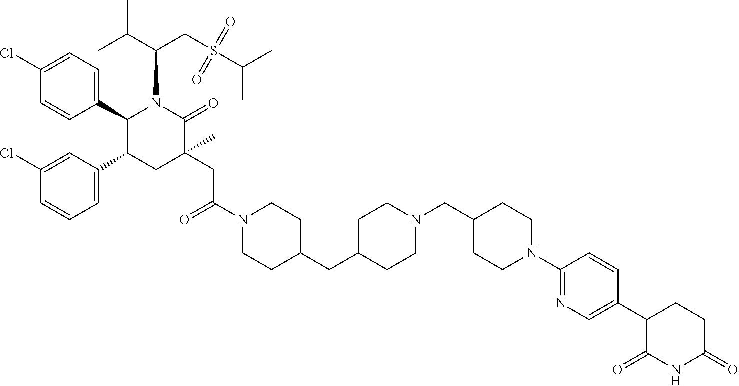 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cn5)CC4)CC3)CC2)C[C@H](c2cccc(Cl)c2)[C@H]1c1ccc(Cl)cc1] |
| 128 | 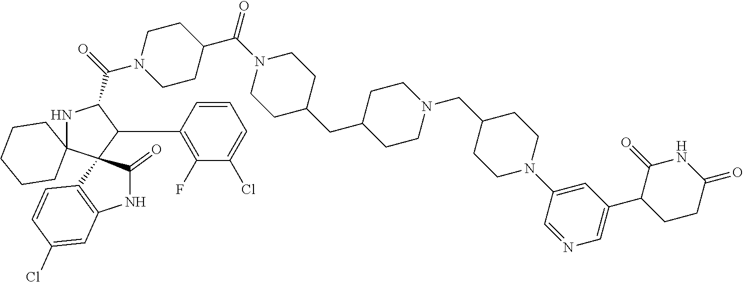 |
| 129 | 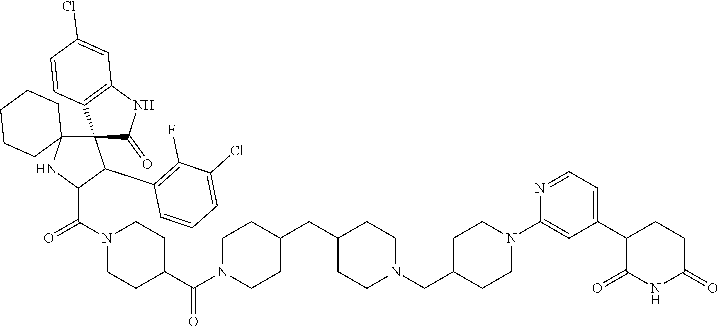 [SMILES: O=C1CCC(c2ccnc(N3CCC(CN4CCC(CC5CCN(C(=O)C6CCN(C(=O)C7NC8(CCCCC8)[C@@]8(C(=O)Nc9cc(Cl)ccc98)C7c7cccc(Cl)c7F)CC6)CC5)CC4)CC3)c2)C(=O)N1] |
| 130 | 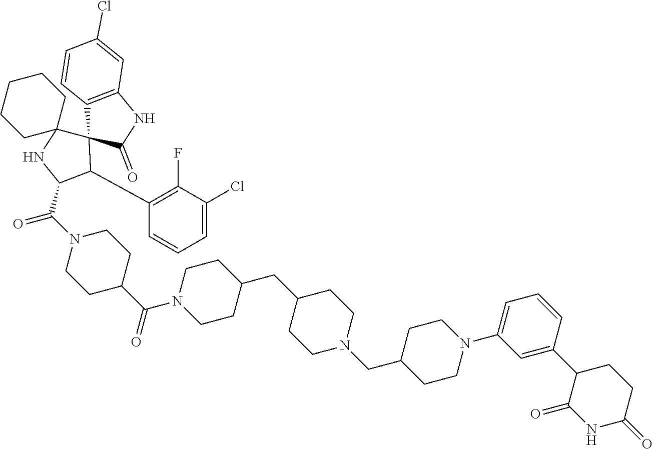 [SMILES: O=C1CCC(c2cccc(N3CCC(CN4CCC(CC5CCN(C(=O)C6CCN(C(=O)[C@@H]7NC8(CCCCC8)[C@@]8(C(=O)Nc9cc(Cl)ccc98)C7c7cccc(Cl)c7F)CC6)CC5)CC4)CC3)c2)C(=O)N1] |
| 131 | 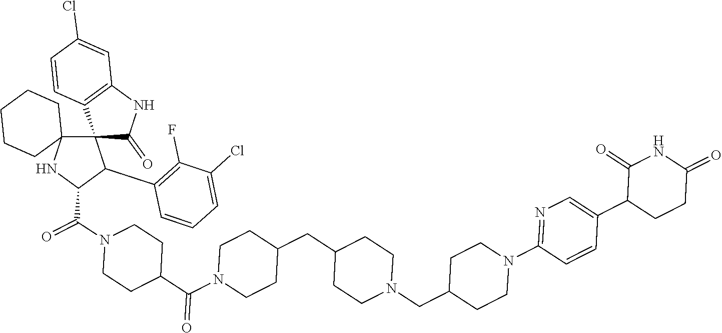 [SMILES: O=C1CCC(c2ccc(N3CCC(CN4CCC(CC5CCN(C(=O)C6CCN(C(=O)[C@@H]7NC8(CCCCC8)[C@@]8(C(=O)Nc9cc(Cl)ccc98)C7c7cccc(Cl)c7F)CC6)CC5)CC4)CC3)nc2)C(=O)N1] |
| 132 | 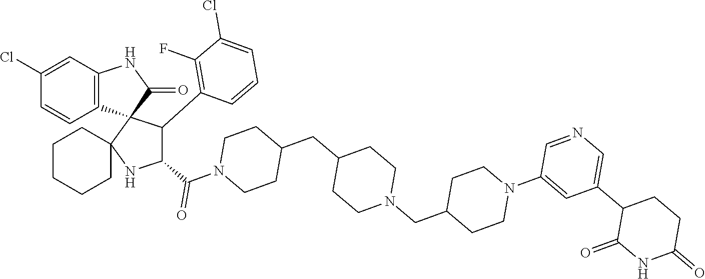 [SMILES: O=C1CCC(c2cncc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)c2)C(=O)N1] |
| 133 | 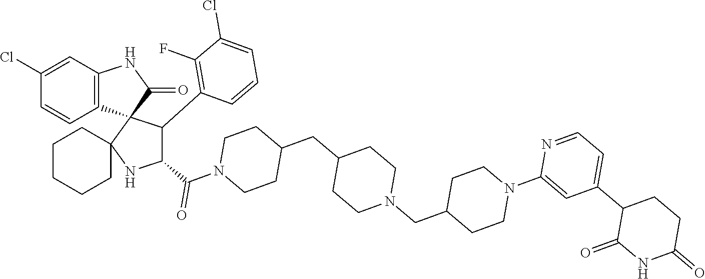 [SMILES: O=C1CCC(c2ccnc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)c2)C(=O)N1] |
| 134 | 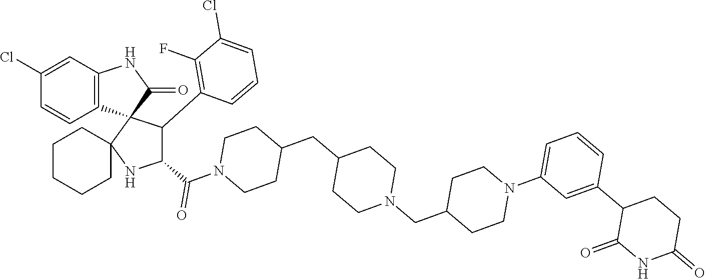 [SMILES: O=C1CCC(c2cccc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)c2)C(=O)N1] |
| 135 | 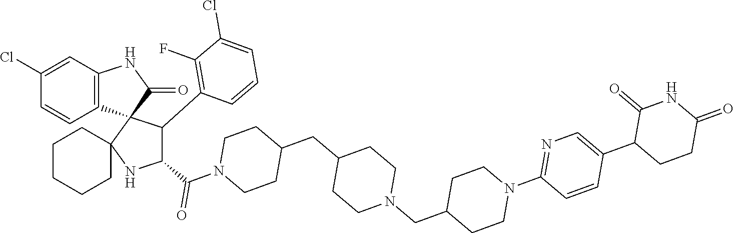 [SMILES: O=C1CCC(c2ccc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)nc2)C(=O)N1] |
| 136 | 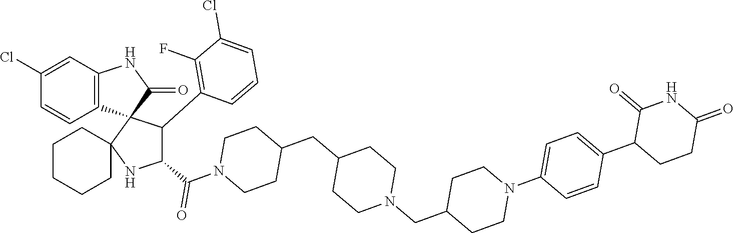 [SMILES: O=C1CCC(c2ccc(N3CCC(CN4CCC(CC5CCN(C(=O)[C@@H]6NC7(CCCCC7)[C@@]7(C(=O)Nc8cc(Cl)ccc87)C6c6cccc(Cl)c6F)CC5)CC4)CC3)cc2)C(=O)N1] |
| 137 | 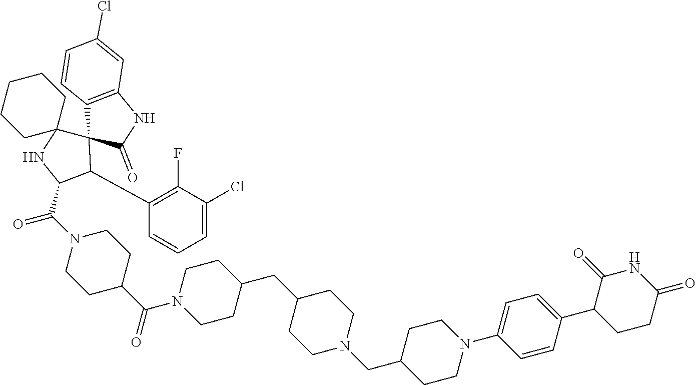 [SMILES: O=C1CCC(c2ccc(N3CCC(CN4CCC(CC5CCN(C(=O)C6CCN(C(=O)[C@@H]7NC8(CCCCC8)[C@@]8(C(=O)Nc9cc(Cl)ccc98)C7c7cccc(Cl)c7F)CC6)CC5)CC4)CC3)cc2)C(=O)N1] |
| 138 | 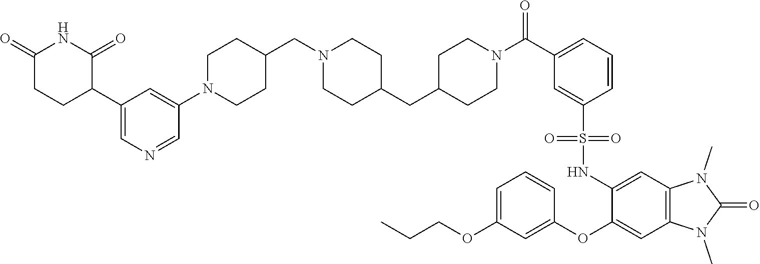 [SMILES: CCCOc1cccc(Oc2cc3c(cc2NS(=O)(=O)c2cccc(C(=O)N4CCC(CC5CCN(CC6CCN(c7cncc(C8CCC(=O)NC8=O)c7)CC6)CC5)CC4)c2)n(C)c(=O)n3C)c1] |
| 139 | 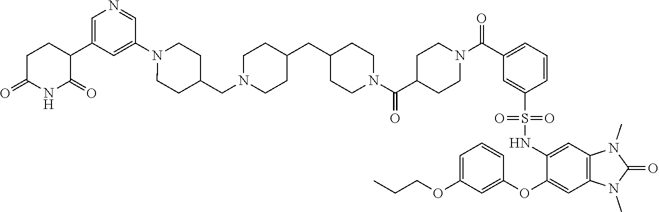 [SMILES: CCCOc1cccc(Oc2cc3c(cc2NS(=O)(=O)c2cccc(C(=O)N4CCC(C(=O)N5CCC(CC6CCN(CC7CCN(c8cncc(C9CCC(=O)NC9=O)c8)CC7)CC6)CC5)CC4)c2)n(C)c(=O)n3C)c1] |
| 140 | 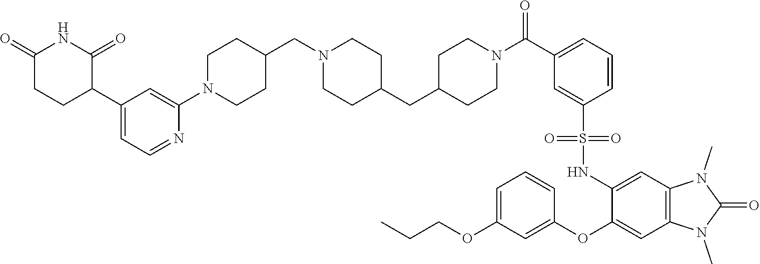 [SMILES: CCCOc1cccc(Oc2cc3c(cc2NS(=O)(=O)c2cccc(C(=O)N4CCC(CC5CCN(CC6CCN(c7cc(C8CCC(=O)NC8=O)ccn7)CC6)CC5)CC4)c2)n(C)c(=O)n3C)c1] |
| 141 | 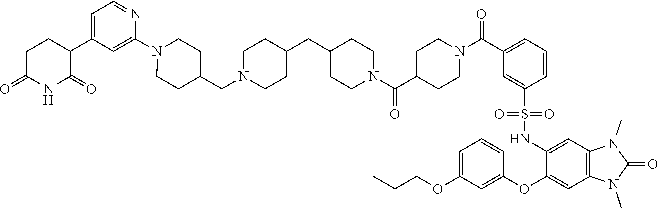 [SMILES: CCCOc1cccc(Oc2cc3c(cc2NS(=O)(=O)c2cccc(C(=O)N4CCC(C(=O)N5CCC(CC6CCN(CC7CCN(c8cc(C9CCC(=O)NC9=O)ccn8)CC7)CC6)CC5)CC4)c2)n(C)c(=O)n3C)c1] |
| 142 | 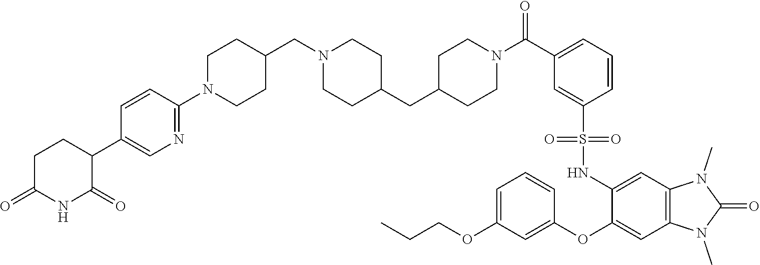 [SMILES: CCCOc1cccc(Oc2cc3c(cc2NS(=O)(=O)c2cccc(C(=O)N4CCC(CC5CCN(CC6CCN(c7ccc(C8CCC(=O)NC8=O)cn7)CC6)CC5)CC4)c2)n(C)c(=O)n3C)c1] |
| 143 | 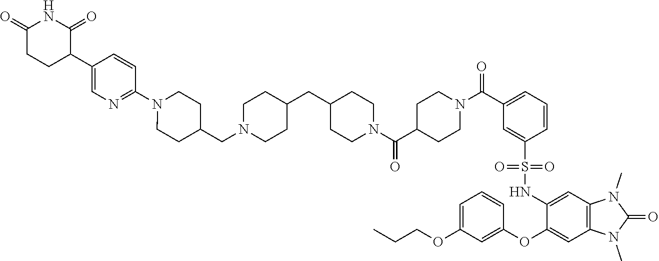 [SMILES: CCCOc1cccc(Oc2cc3c(cc2NS(=O)(=O)c2cccc(C(=O)N4CCC(C(=O)N5CCC(CC6CCN(CC7CCN(c8ccc(C9CCC(=O)NC9=O)cn8)CC7)CC6)CC5)CC4)c2)n(C)c(=O)n3C)c1] |
| 144 | 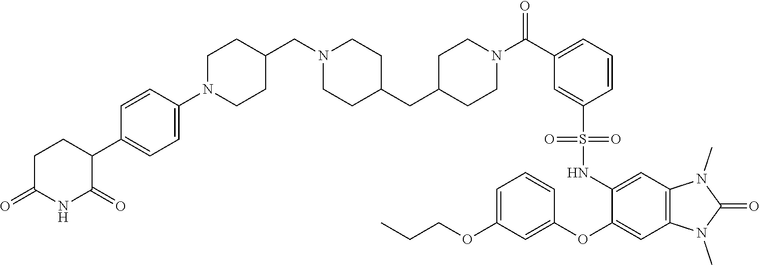 [SMILES: CCCOc1cccc(Oc2cc3c(cc2NS(=O)(=O)c2cccc(C(=O)N4CCC(CC5CCN(CC6CCN(c7ccc(C8CCC(=O)NC8=O)cc7)CC6)CC5)CC4)c2)n(C)c(=O)n3C)c1] |
| 145 | 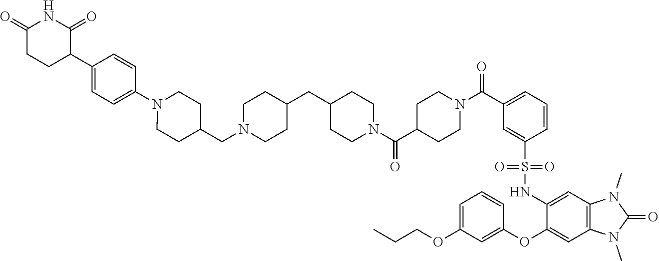 [SMILES: CCCOc1cccc(Oc2cc3c(cc2NS(=O)(=O)c2cccc(C(=O)N4CCC(C(=O)N5CCC(CC6CCN(CC7CCN(c8ccc(C9CCC(=O)NC9=O)cc8)CC7)CC6)CC5)CC4)c2)n(C)c(=O)n3C)c1] |
| 146 | 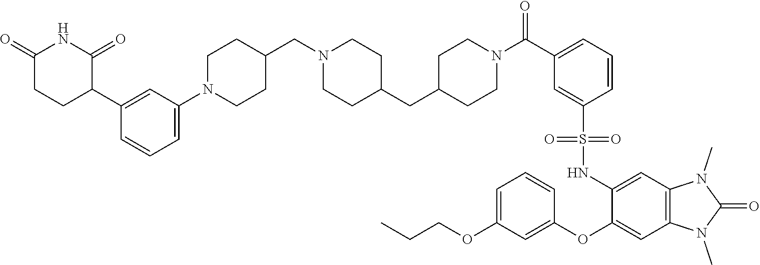 [SMILES: CCCOc1cccc(Oc2cc3c(cc2NS(=O)(=O)c2cccc(C(=O)N4CCC(CC5CCN(CC6CCN(c7cccc(C8CCC(=O)NC8=O)c7)CC6)CC5)CC4)c2)n(C)c(=O)n3C)c1] |
| 147 | 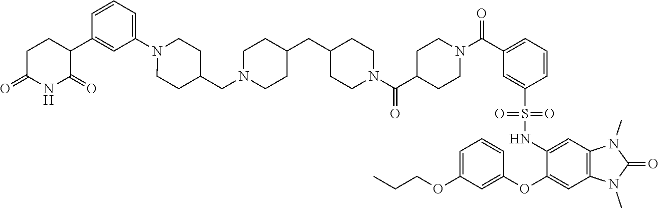 [SMILES: CCCOc1cccc(Oc2cc3c(cc2NS(=O)(=O)c2cccc(C(=O)N4CCC(C(=O)N5CCC(CC6CCN(CC7CCN(c8cccc(C9CCC(=O)NC9=O)c8)CC7)CC6)CC5)CC4)c2)n(C)c(=O)n3C)c1] |
| 148 | 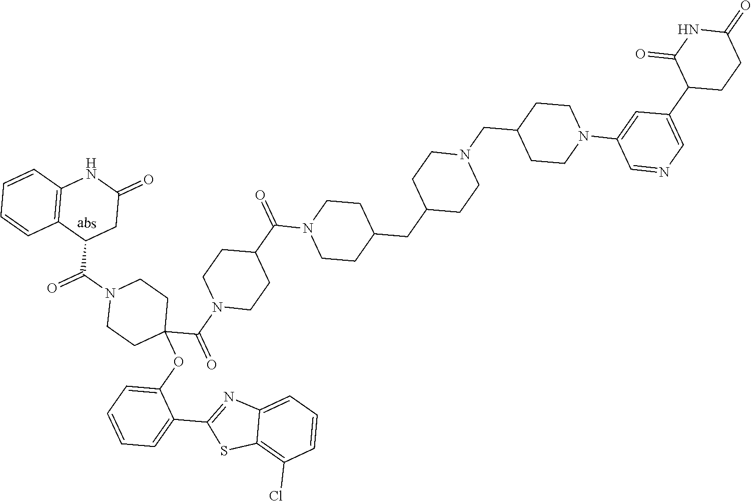 [SMILES: O=C1CCC(c2cncc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)c2)C(=O)N1] |
| 149 | 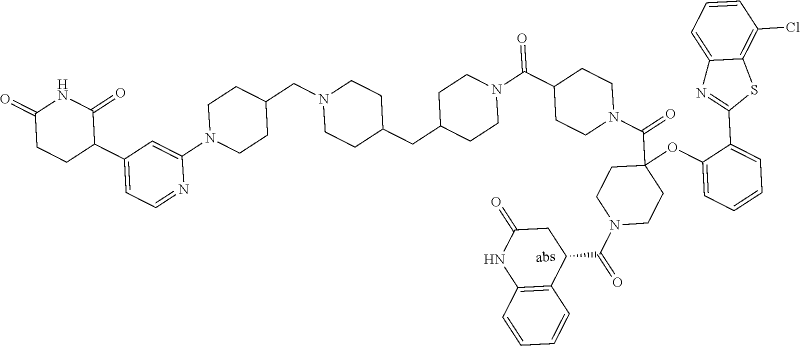 [SMILES: O=C1CCC(c2ccnc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)c2)C(=O)N1] |
| 150 | 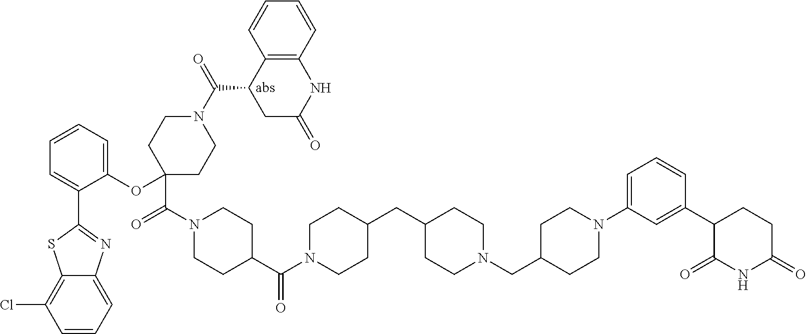 [SMILES: O=C1CCC(c2cccc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)c2)C(=O)N1] |
| 151 | 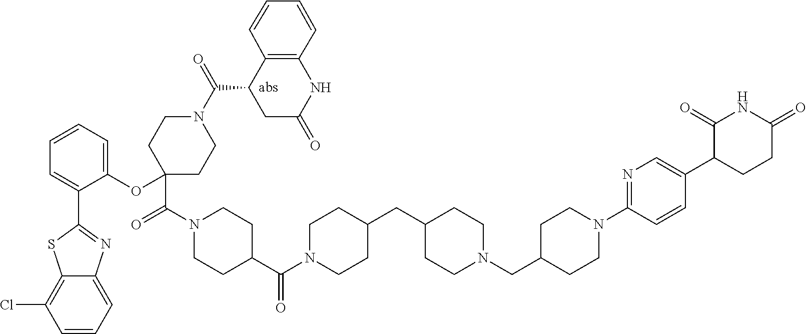 [SMILES: O=C1CCC(c2ccc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)nc2)C(=O)N1] |
| 152 | 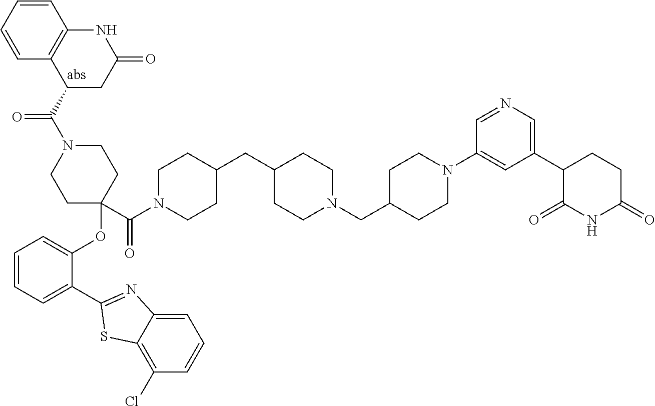 [SMILES: O=C1CCC(c2cncc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] |
| 153 | 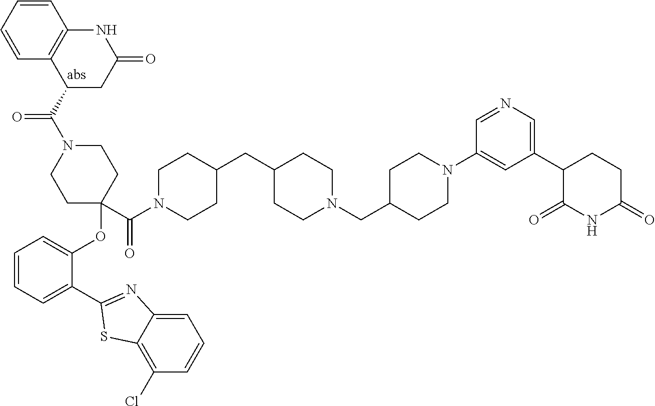 [SMILES: O=C1CCC(c2cncc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] |
| 154 | 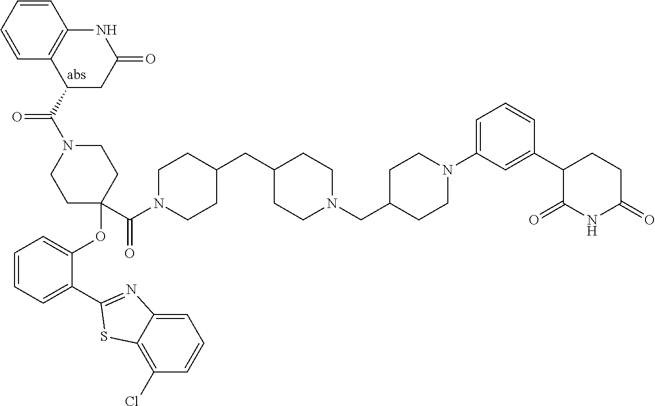 [SMILES: O=C1CCC(c2cccc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] |
| 155 | 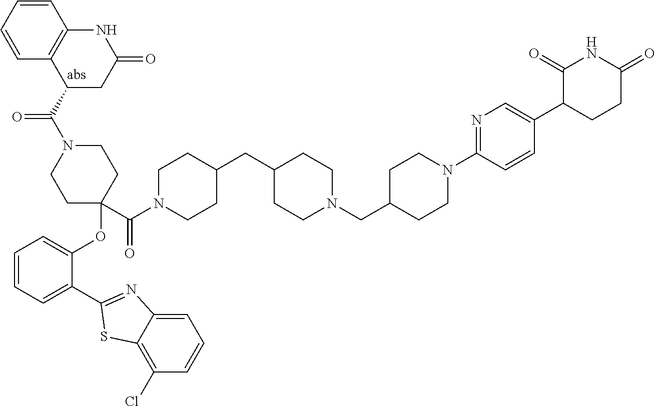 [SMILES: O=C1CCC(c2ccc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)nc2)C(=O)N1] |
| 156 | 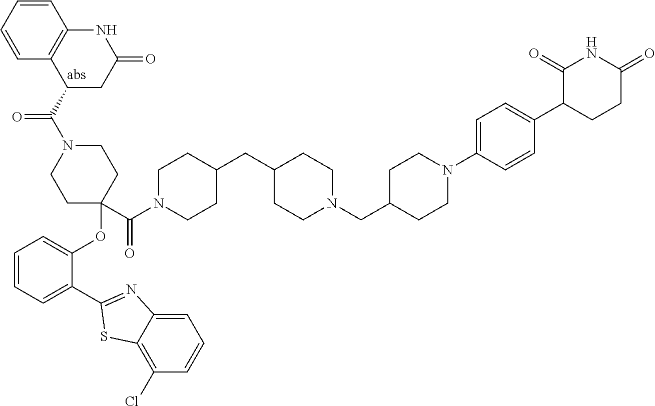 [SMILES: O=C1CCC(c2ccc(N3CCC(CN4CCC(CC5CCN(C(=O)C6(Oc7ccccc7-c7nc8cccc(Cl)c8s7)CCN(C(=O)[C@H]7CC(=O)Nc8ccccc87)CC6)CC5)CC4)CC3)cc2)C(=O)N1] |
| 157 | 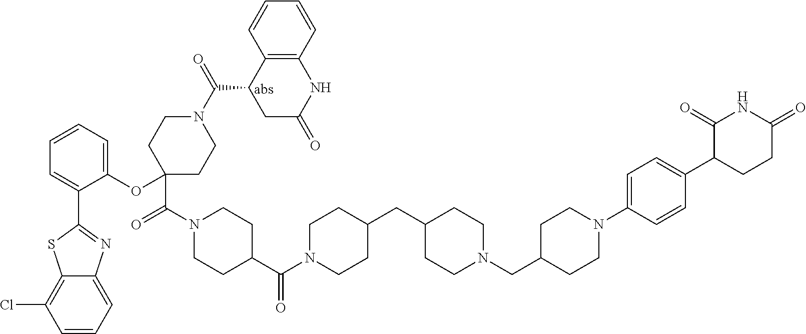 [SMILES: O=C1CCC(c2ccc(N3CCC(CN4CCC(CC5CCN(C(=O)C6CCN(C(=O)C7(Oc8ccccc8-c8nc9cccc(Cl)c9s8)CCN(C(=O)[C@H]8CC(=O)Nc9ccccc98)CC7)CC6)CC5)CC4)CC3)cc2)C(=O)N1] |
| 158 | 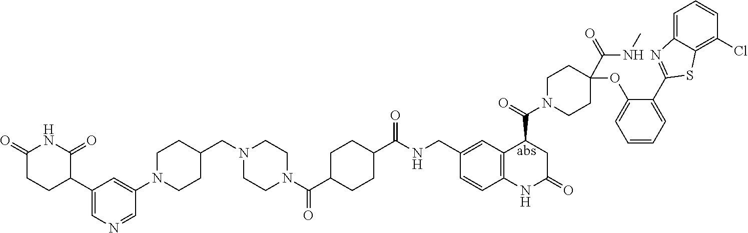 [SMILES: CNC(=O)C1(Oc2ccccc2-c2nc3cccc(Cl)c3s2)CCN(C(=O)[C@H]2CC(=O)Nc3ccc(CNC(=O)C4CCC(C(=O)N5CCN(CC6CCN(c7cncc(C8CCC(=O)NC8=O)c7)CC6)CC5)CC4)cc32)CC1] |
| 159 | 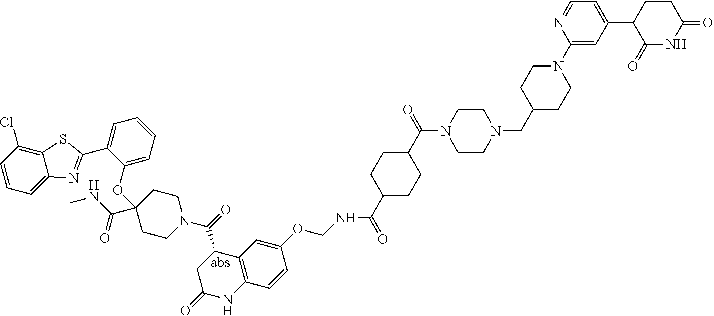 [SMILES: CNC(=O)C1(Oc2ccccc2-c2nc3cccc(Cl)c3s2)CCN(C(=O)[C@H]2CC(=O)Nc3ccc(OCNC(=O)C4CCC(C(=O)N5CCN(CC6CCN(c7cc(C8CCC(=O)NC8=O)ccn7)CC6)CC5)CC4)cc32)CC1] |
| 160 | 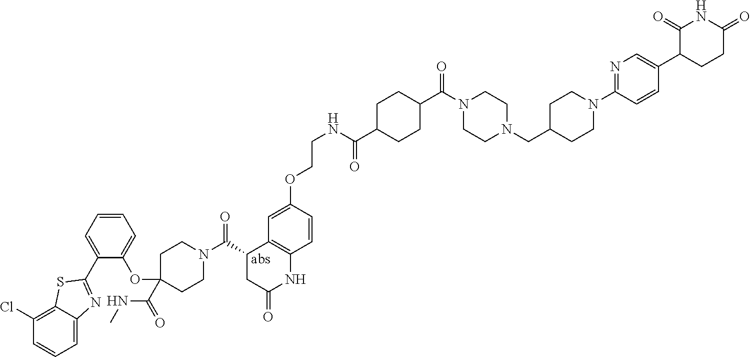 [SMILES: CNC(=O)C1(Oc2ccccc2-c2nc3cccc(Cl)c3s2)CCN(C(=O)[C@H]2CC(=O)Nc3ccc(OCCNC(=O)C4CCC(C(=O)N5CCN(CC6CCN(c7ccc(C8CCC(=O)NC8=O)cn7)CC6)CC5)CC4)cc32)CC1] |
| 161 | 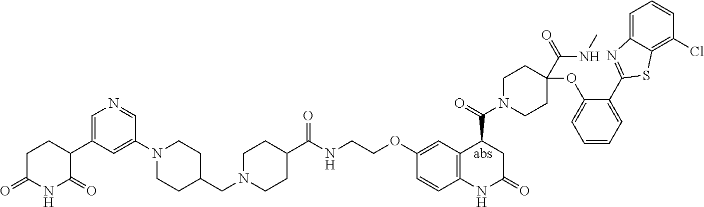 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6cncc(C7CCC(=O)NC7=O)c6)CC5)CC4)cc32)CC1] |
| 162 | 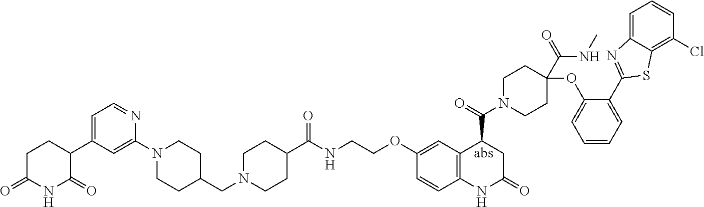 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6cc(C7CCC(=O)NC7=O)ccn6)CC5)CC4)cc32)CC1] |
| 163 | 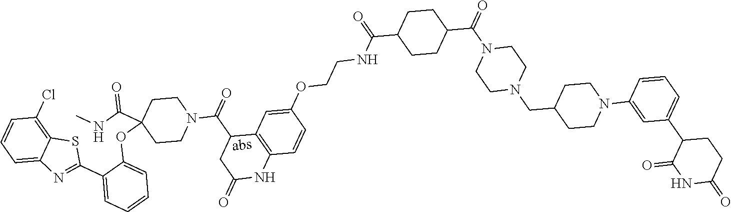 [SMILES: CNC(=O)C1(Oc2ccccc2-c2nc3cccc(Cl)c3s2)CCN(C(=O)C2CC(=O)Nc3ccc(OCCNC(=O)C4CCC(C(=O)N5CCN(CC6CCN(c7cccc(C8CCC(=O)NC8=O)c7)CC6)CC5)CC4)cc32)CC1] |
| 164 | 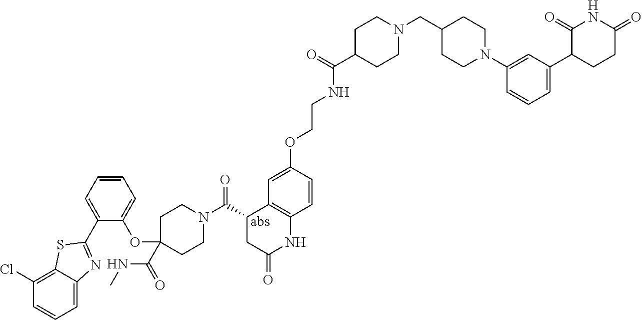 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6cccc(C7CCC(=O)NC7=O)c6)CC5)CC4)cc32)CC1] |
| 165 | 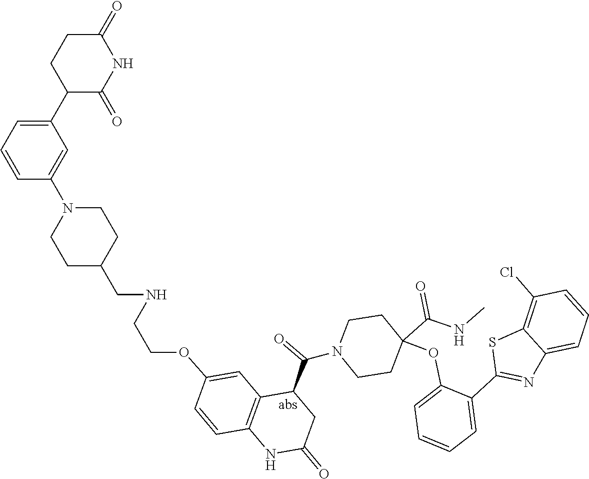 [SMILES: CNC(=O)C1(Oc2ccccc2-c2nc3cccc(Cl)c3s2)CCN(C(=O)[C@H]2CC(=O)Nc3ccc(OCCNCC4CCN(c5cccc(C6CCC(=O)NC6=O)c5)CC4)cc32)CC1] |
| 166 | 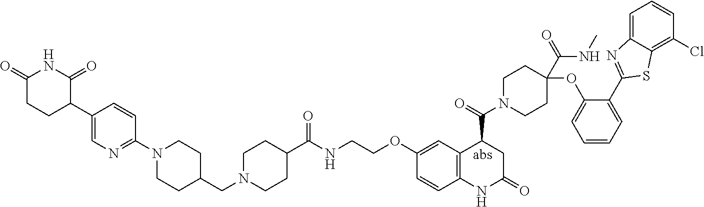 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6ccc(C7CCC(=O)NC7=O)cn6)CC5)CC4)cc32)CC1] |
| 167 | 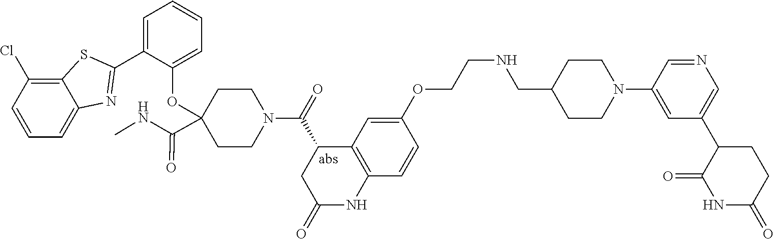 [SMILES: CNC(=O)C1(Oc2ccccc2-c2nc3cccc(Cl)c3s2)CCN(C(=O)[C@H]2CC(=O)Nc3ccc(OCCNCC4CCN(c5cncc(C6CCC(=O)NC6=O)c5)CC4)cc32)CC1] |
| 168 | 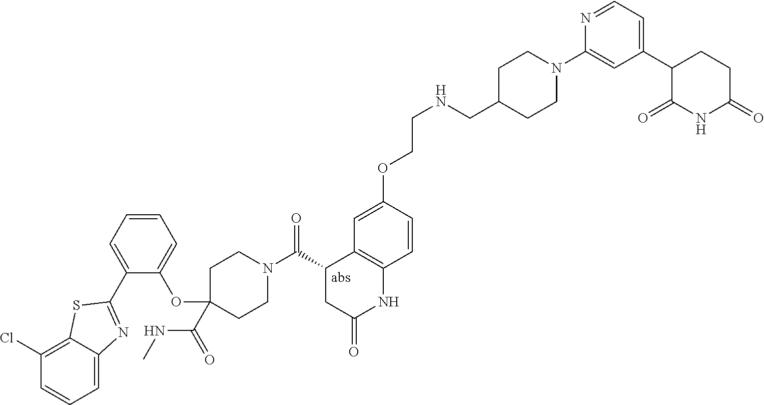 [SMILES: CNC(=O)C1(Oc2ccccc2-c2nc3cccc(Cl)c3s2)CCN(C(=O)[C@H]2CC(=O)Nc3ccc(OCCNCC4CCN(c5cc(C6CCC(=O)NC6=O)ccn5)CC4)cc32)CC1] |
| 169 | 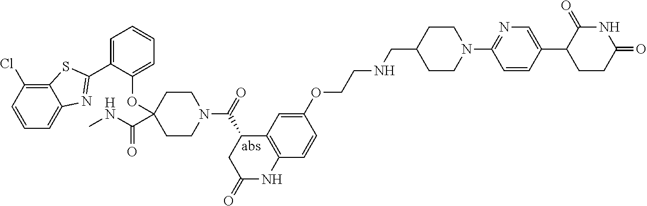 [SMILES: CNC(=O)C1(Oc2ccccc2-c2nc3cccc(Cl)c3s2)CCN(C(=O)[C@H]2CC(=O)Nc3ccc(OCCNCC4CCN(c5ccc(C6CCC(=O)NC6=O)cn5)CC4)cc32)CC1] |
| 170 | 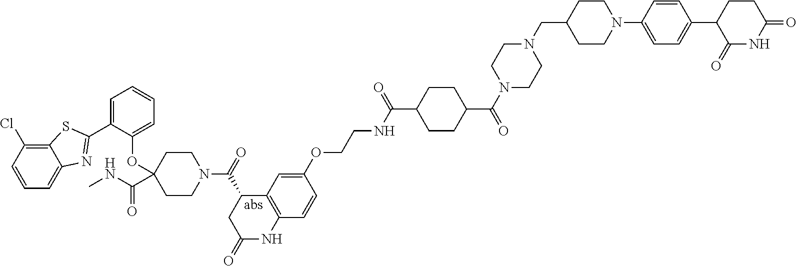 [SMILES: CNC(=O)C1(Oc2ccccc2-c2nc3cccc(Cl)c3s2)CCN(C(=O)[C@H]2CC(=O)Nc3ccc(OCCNC(=O)C4CCC(C(=O)N5CCN(CC6CCN(c7ccc(C8CCC(=O)NC8=O)cc7)CC6)CC5)CC4)cc32)CC1] |
| 171 | 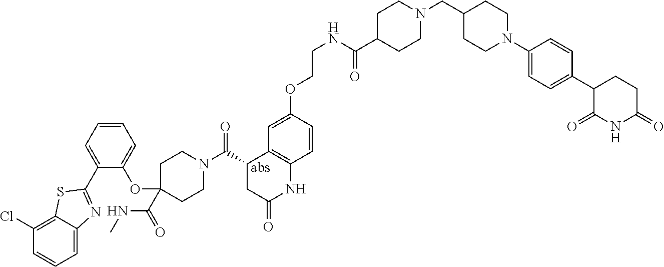 [SMILES: CNC(=O)C1(Oc2ccccc2-c2nc3cccc(Cl)c3s2)CCN(C(=O)[C@H]2CC(=O)Nc3ccc(OCCNC(=O)C4CCN(CC5CCN(c6ccc(C7CCC(=O)NC7=O)cc6)CC5)CC4)cc32)CC1] |
| 172 | 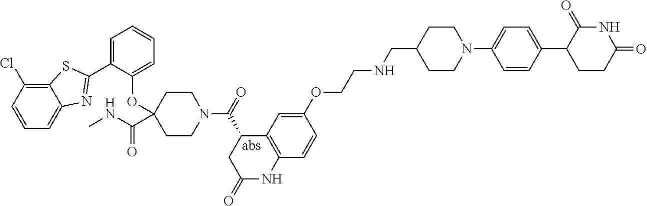 [SMILES: CNC(=O)C1(Oc2ccccc2-c2nc3cccc(Cl)c3s2)CCN(C(=O)[C@H]2CC(=O)Nc3ccc(OCCNCC4CCN(c5ccc(C6CCC(=O)NC6=O)cc5)CC4)cc32)CC1] |
| 173 | 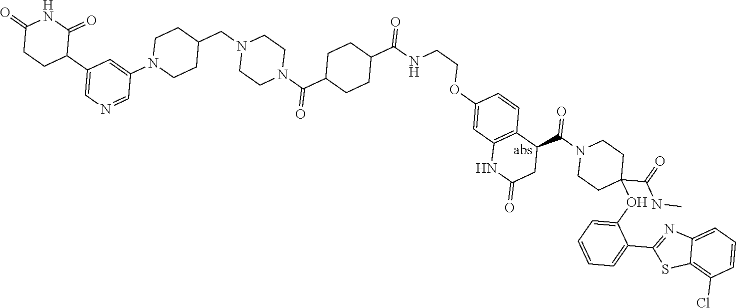 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7cncc(C8CCC(=O)NC8=O)c7)CC6)CC5)CC4)ccc32)CC1] |
| 174 | 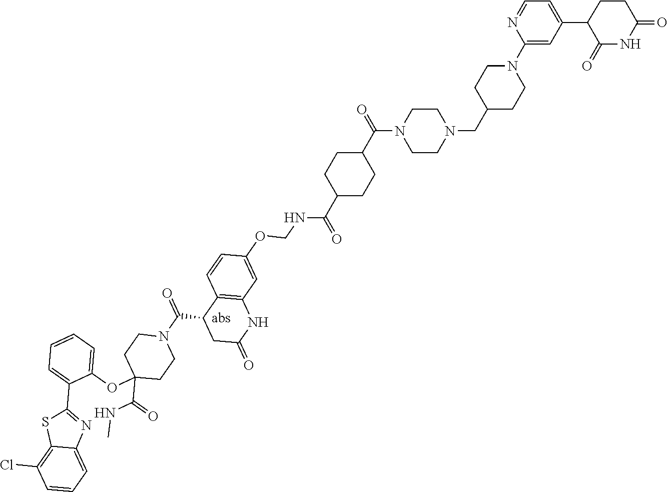 [SMILES: CNC(=O)C1(Oc2ccccc2-c2nc3cccc(Cl)c3s2)CCN(C(=O)[C@H]2CC(=O)Nc3cc(OCNC(=O)C4CCC(C(=O)N5CCN(CC6CCN(c7cc(C8CCC(=O)NC8=O)ccn7)CC6)CC5)CC4)ccc32)CC1] |
| 175 | 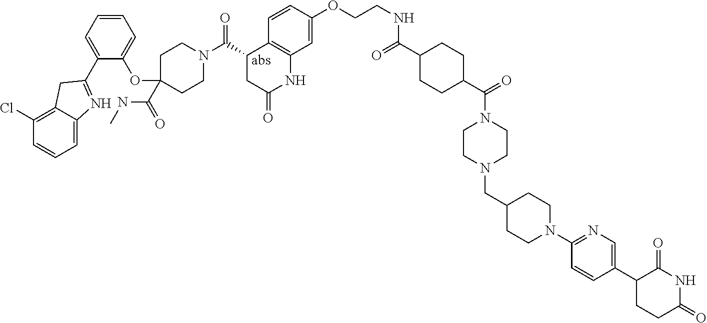 [SMILES: CNC(=O)C1(Oc2ccccc2C2=Nc3cccc(Cl)c3C2)CCN(C(=O)[C@H]2CC(=O)Nc3cc(OCCNC(=O)C4CCC(C(=O)N5CCN(CC6CCN(c7ccc(C8CCC(=O)NC8=O)cn7)CC6)CC5)CC4)ccc32)CC1] |
| 176 | 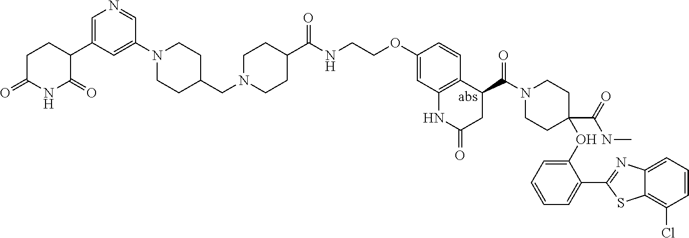 |
| 177 | 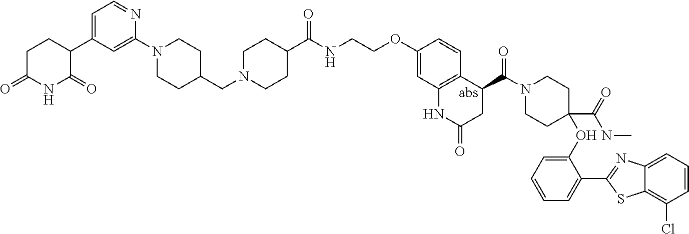 |
| 178 | 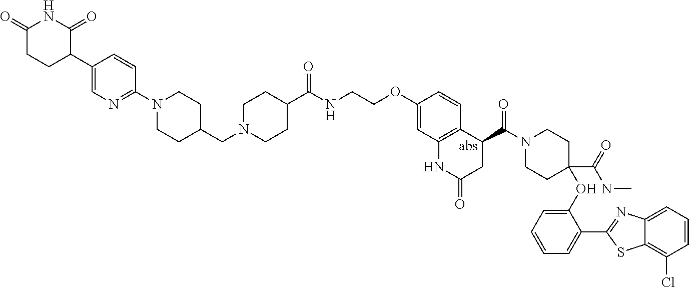 |
| 179 | 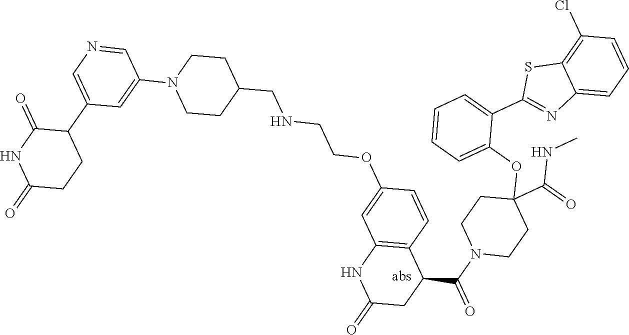 [SMILES: CNC(=O)C1(Oc2ccccc2-c2nc3cccc(Cl)c3s2)CCN(C(=O)[C@H]2CC(=O)Nc3cc(OCCNCC4CCN(c5cncc(C6CCC(=O)NC6=O)c5)CC4)ccc32)CC1] |
| 180 | 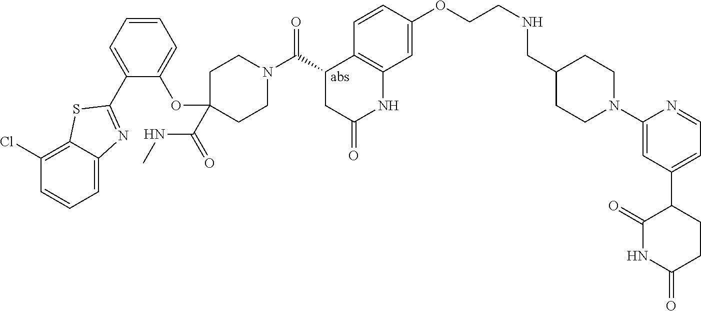 [SMILES: CNC(=O)C1(Oc2ccccc2-c2nc3cccc(Cl)c3s2)CCN(C(=O)[C@H]2CC(=O)Nc3cc(OCCNCC4CCN(c5cc(C6CCC(=O)NC6=O)ccn5)CC4)ccc32)CC1] |
| 181 | 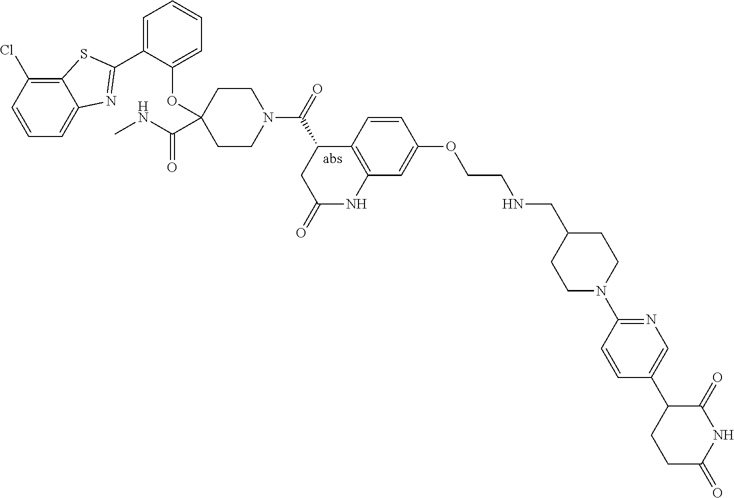 [SMILES: CNC(=O)C1(Oc2ccccc2-c2nc3cccc(Cl)c3s2)CCN(C(=O)[C@H]2CC(=O)Nc3cc(OCCNCC4CCN(c5ccc(C6CCC(=O)NC6=O)cn5)CC4)ccc32)CC1] |
| 182 | 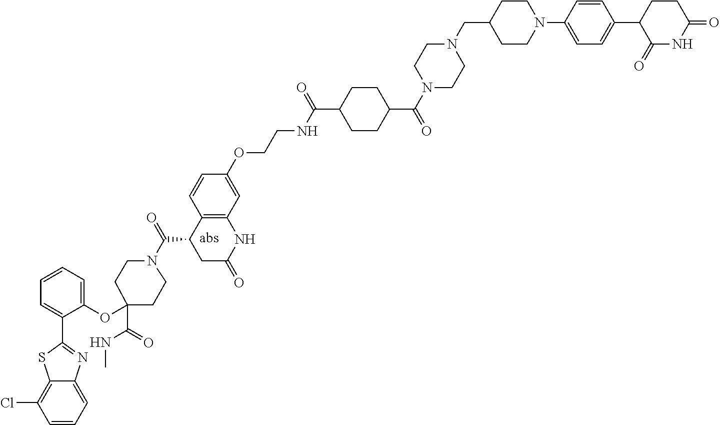 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7ccc(C8CCC(=O)NC8=O)cc7)CC6)CC5)CC4)ccc32)CC1] |
| 183 | 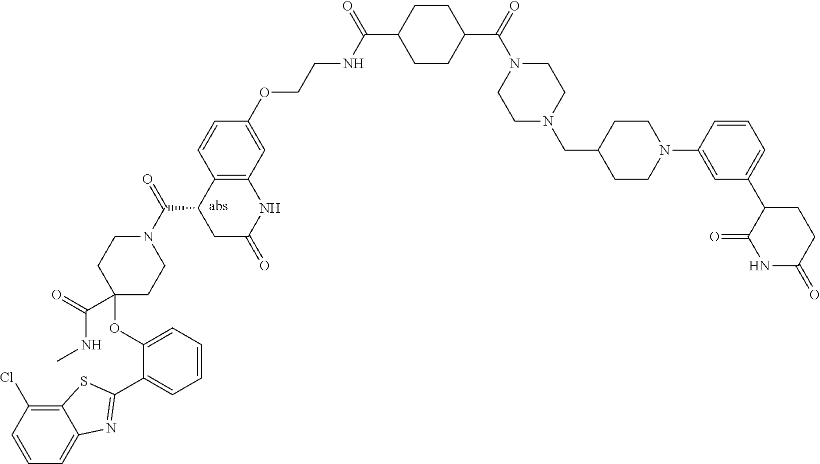 [SMILES: CNC(=O)C1(Oc2ccccc2-c2nc3cccc(Cl)c3s2)CCN(C(=O)[C@H]2CC(=O)Nc3cc(OCCNC(=O)C4CCC(C(=O)N5CCN(CC6CCN(c7cccc(C8CCC(=O)NC8=O)c7)CC6)CC5)CC4)ccc32)CC1] |
| 184 | 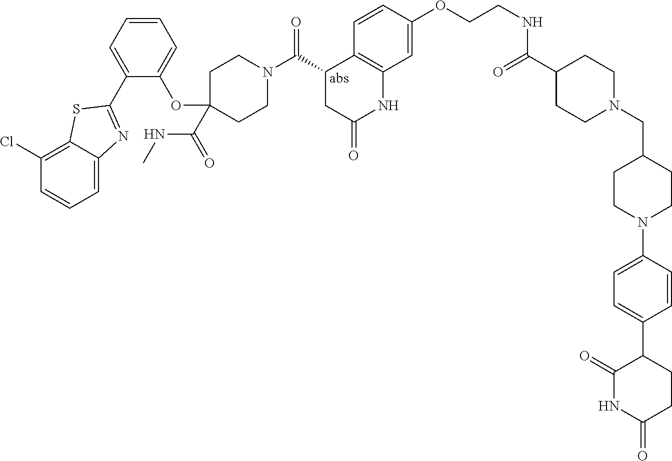 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6ccc(C7CCC(=O)NC7=O)cc6)CC5)CC4)ccc32)CC1] |
| 185 | 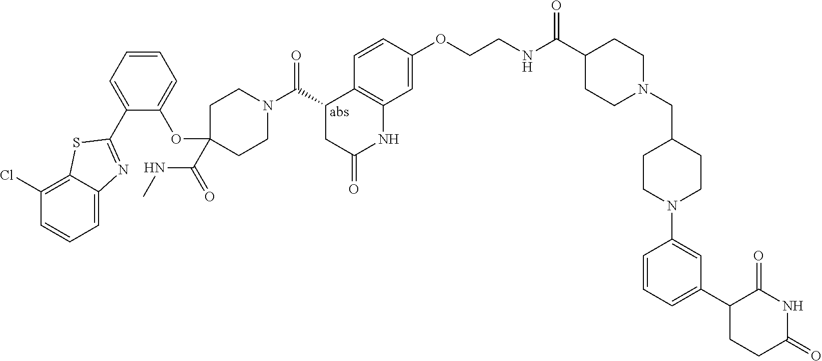 [SMILES: CNC(=O)C1(Oc2ccccc2-c2nc3cccc(Cl)c3s2)CCN(C(=O)[C@H]2CC(=O)Nc3cc(OCCNC(=O)C4CCN(CC5CCN(c6cccc(C7CCC(=O)NC7=O)c6)CC5)CC4)ccc32)CC1] |
| 186 | 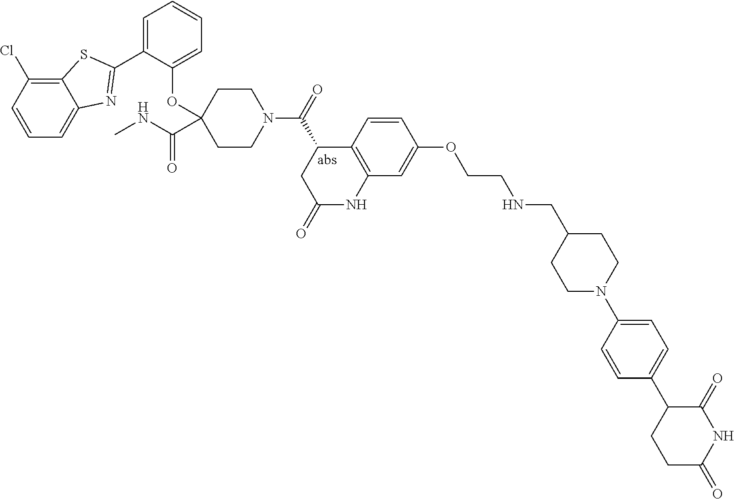 [SMILES: CNC(=O)C1(Oc2ccccc2-c2nc3cccc(Cl)c3s2)CCN(C(=O)[C@H]2CC(=O)Nc3cc(OCCNCC4CCN(c5ccc(C6CCC(=O)NC6=O)cc5)CC4)ccc32)CC1] |
| 187 | 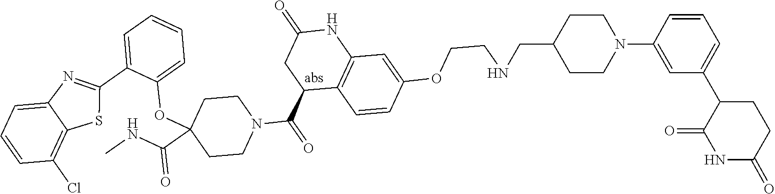 [SMILES: CNC(=O)C1(Oc2ccccc2-c2nc3cccc(Cl)c3s2)CCN(C(=O)[C@H]2CC(=O)Nc3cc(OCCNCC4CCN(c5cccc(C6CCC(=O)NC6=O)c5)CC4)ccc32)CC1] |
| 188 | 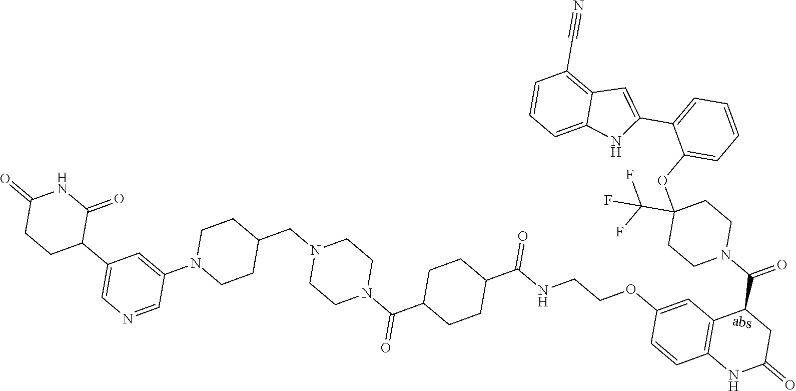 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cncc(C%10CCC(=O)NC%10=O)c9)CC8)CC7)CC6)cc54)CC3)cc12] |
| 189 | 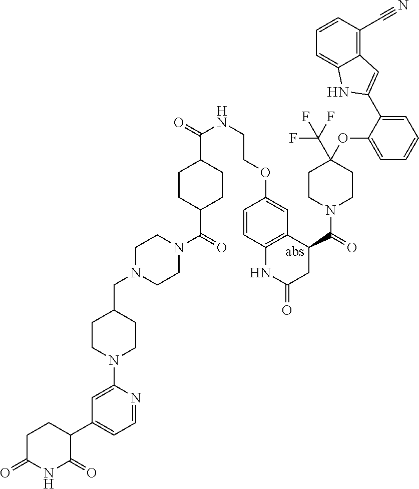 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cc(C%10CCC(=O)NC%10=O)ccn9)CC8)CC7)CC6)cc54)CC3)cc12] |
| 190 | 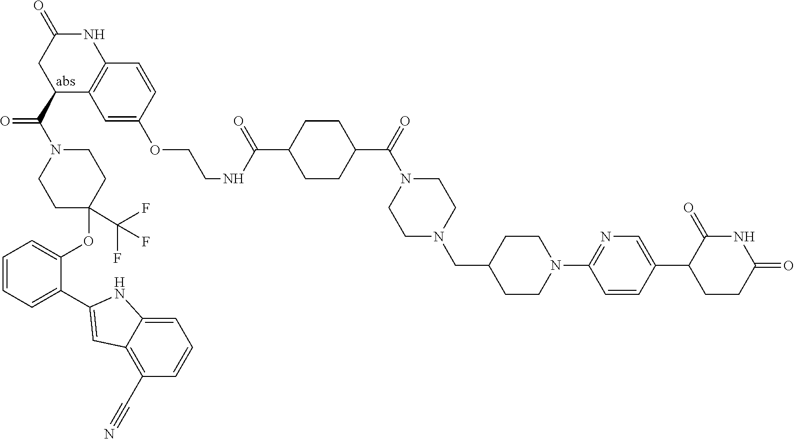 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cn9)CC8)CC7)CC6)cc54)CC3)cc12] |
| 191 | 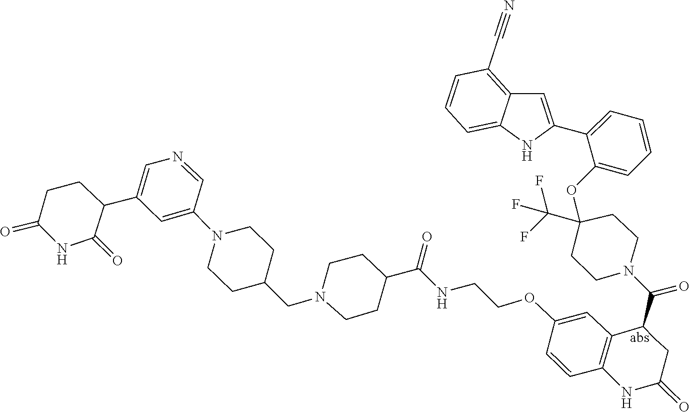 |
| 192 | 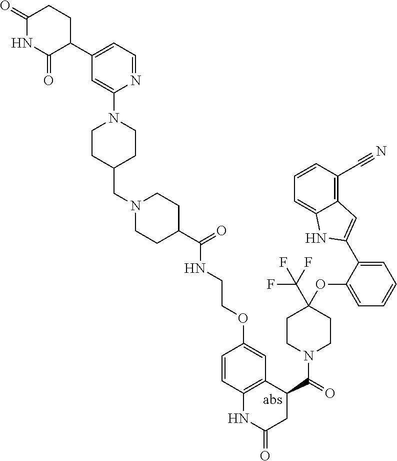 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cc(C9CCC(=O)NC9=O)ccn8)CC7)CC6)cc54)CC3)cc12] |
| 193 | 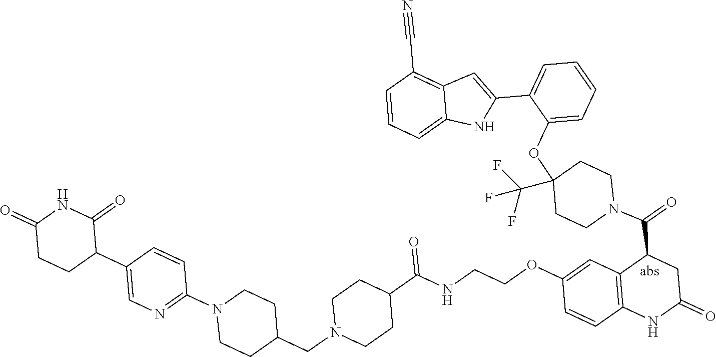 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cn8)CC7)CC6)cc54)CC3)cc12] |
| 194 | 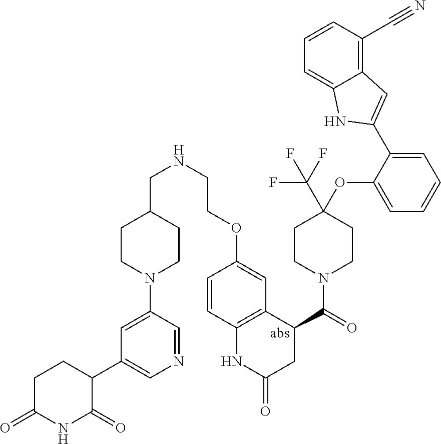 [SMILES: N#Cc1cccc2[nH]c(-c3ccccc3OC3(C(F)(F)F)CCN(C(=O)[C@H]4CC(=O)Nc5ccc(OCCNCC6CCN(c7cncc(C8CCC(=O)NC8=O)c7)CC6)cc54)CC3)cc12] |
| 195 | 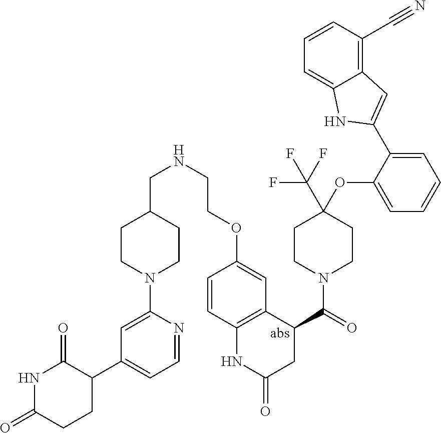 [SMILES: N#Cc1cccc2[nH]c(-c3ccccc3OC3(C(F)(F)F)CCN(C(=O)[C@H]4CC(=O)Nc5ccc(OCCNCC6CCN(c7cc(C8CCC(=O)NC8=O)ccn7)CC6)cc54)CC3)cc12] |
| 196 | 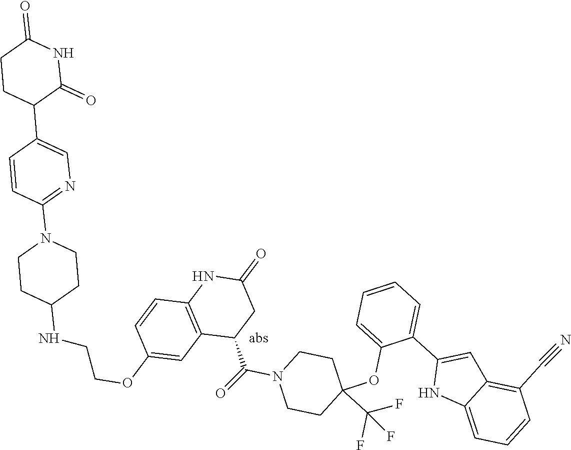 [SMILES: N#Cc1cccc2[nH]c(-c3ccccc3OC3(C(F)(F)F)CCN(C(=O)[C@H]4CC(=O)Nc5ccc(OCCNC6CCN(c7ccc(C8CCC(=O)NC8=O)cn7)CC6)cc54)CC3)cc12] |
| 197 | 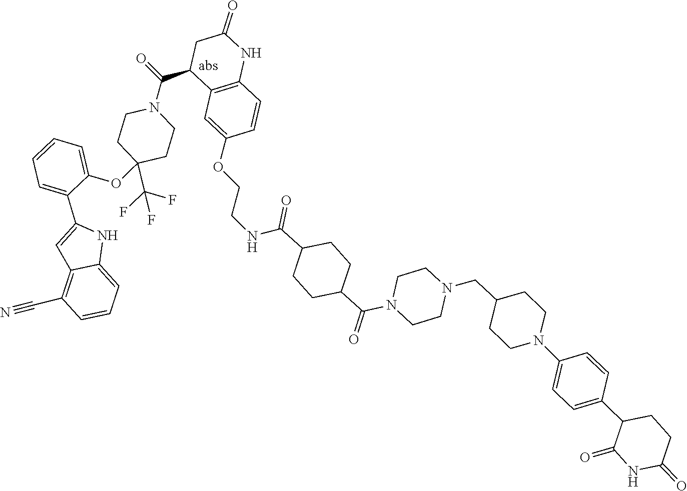 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cc9)CC8)CC7)CC6)cc54)CC3)cc12] |
| 198 | 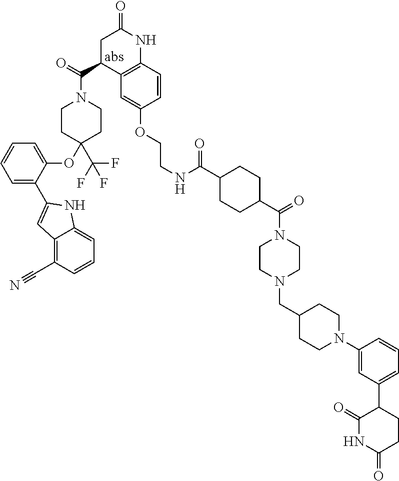 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cccc(C%10CCC(=O)NC%10=O)c9)CC8)CC7)CC6)cc54)CC3)cc12] |
| 199 | 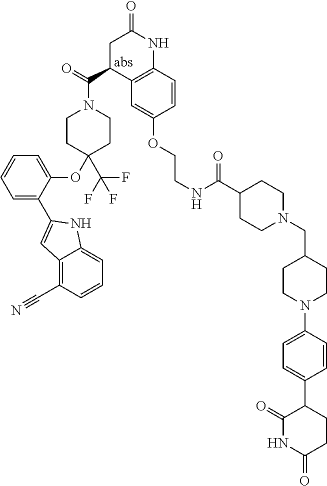 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cc8)CC7)CC6)cc54)CC3)cc12] |
| 200 | 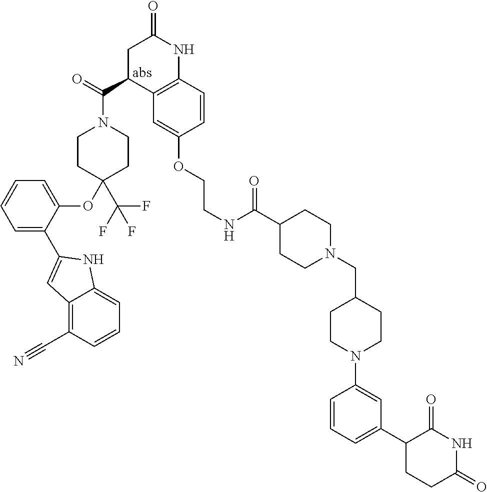 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cccc(C9CCC(=O)NC9=O)c8)CC7)CC6)cc54)CC3)cc12] |
| 201 | 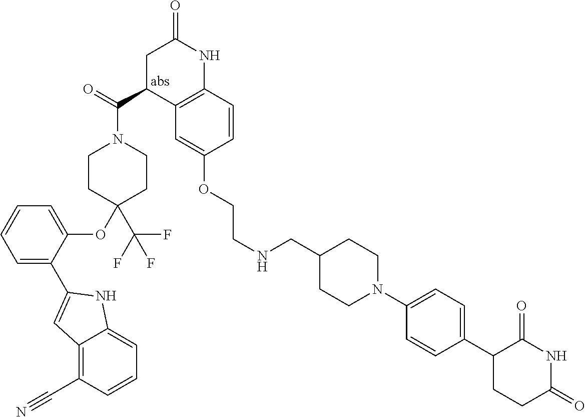 [SMILES: N#Cc1cccc2[nH]c(-c3ccccc3OC3(C(F)(F)F)CCN(C(=O)[C@H]4CC(=O)Nc5ccc(OCCNCC6CCN(c7ccc(C8CCC(=O)NC8=O)cc7)CC6)cc54)CC3)cc12] |
| 202 | 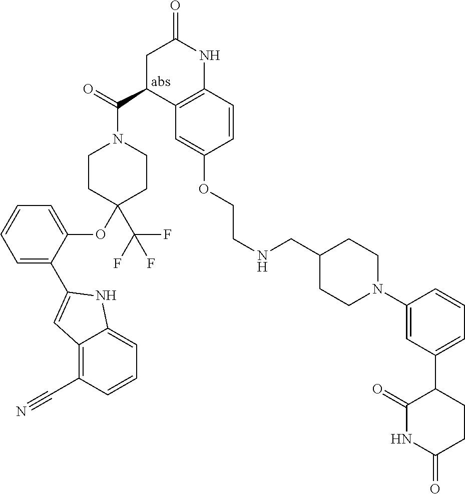 [SMILES: N#Cc1cccc2[nH]c(-c3ccccc3OC3(C(F)(F)F)CCN(C(=O)[C@H]4CC(=O)Nc5ccc(OCCNCC6CCN(c7cccc(C8CCC(=O)NC8=O)c7)CC6)cc54)CC3)cc12] |
| 203 | 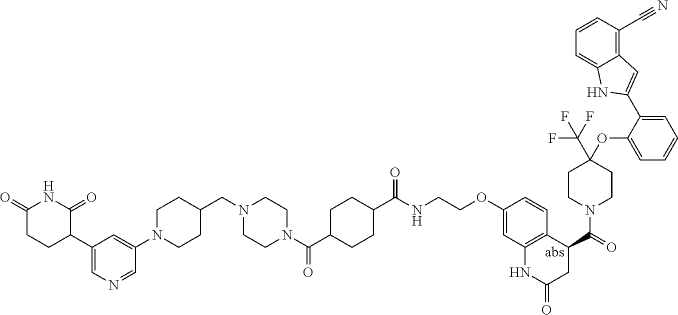 [SMILES: N#Cc1cccc2[nH]c(-c3ccccc3OC3(C(F)(F)F)CCN(C(=O)[C@H]4CC(=O)Nc5cc(OCCNC(=O)C6CCC(C(=O)N7CCN(CC8CCN(c9cncc(C%10CCC(=O)NC%10=O)c9)CC8)CC7)CC6)ccc54)CC3)cc12] |
| 204 | 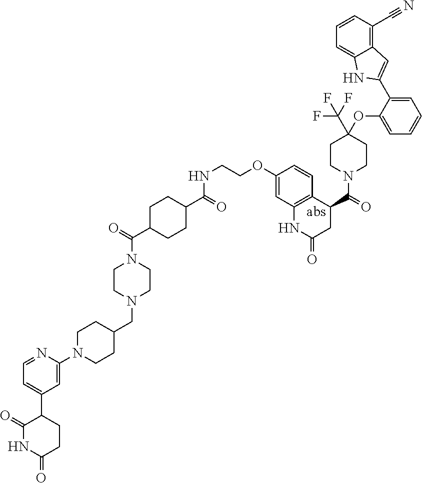 [SMILES: N#Cc1cccc2[nH]c(-c3ccccc3OC3(C(F)(F)F)CCN(C(=O)[C@H]4CC(=O)Nc5cc(OCCNC(=O)C6CCC(C(=O)N7CCN(CC8CCN(c9cc(C%10CCC(=O)NC%10=O)ccn9)CC8)CC7)CC6)ccc54)CC3)cc12] |
| 205 | 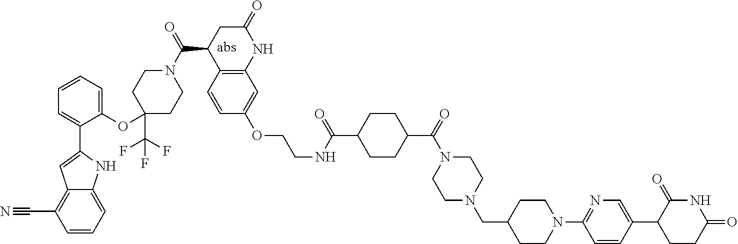 [SMILES: N#Cc1cccc2[nH]c(-c3ccccc3OC3(C(F)(F)F)CCN(C(=O)[C@H]4CC(=O)Nc5cc(OCCNC(=O)C6CCC(C(=O)N7CCN(CC8CCN(c9ccc(C%10CCC(=O)NC%10=O)cn9)CC8)CC7)CC6)ccc54)CC3)cc12] |
| 206 | 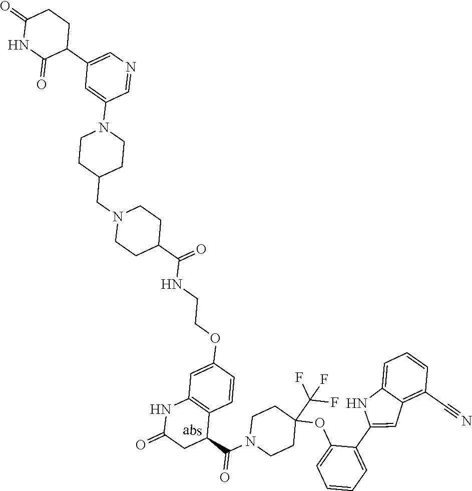 [SMILES: N#Cc1cccc2[nH]c(-c3ccccc3OC3(C(F)(F)F)CCN(C(=O)[C@H]4CC(=O)Nc5cc(OCCNC(=O)C6CCN(CC7CCN(c8cncc(C9CCC(=O)NC9=O)c8)CC7)CC6)ccc54)CC3)cc12] |
| 207 | 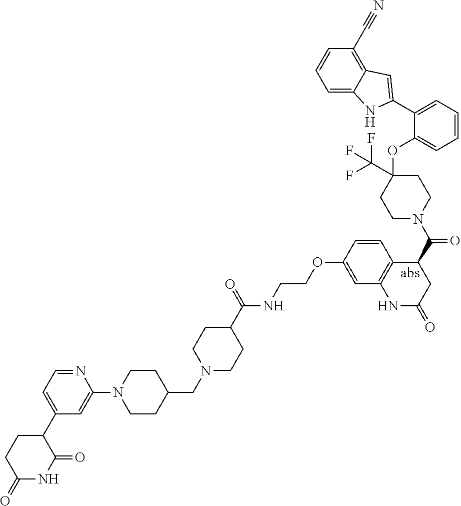 [SMILES: N#Cc1cccc2[nH]c(-c3ccccc3OC3(C(F)(F)F)CCN(C(=O)[C@H]4CC(=O)Nc5cc(OCCNC(=O)C6CCN(CC7CCN(c8cc(C9CCC(=O)NC9=O)ccn8)CC7)CC6)ccc54)CC3)cc12] |
| 208 | 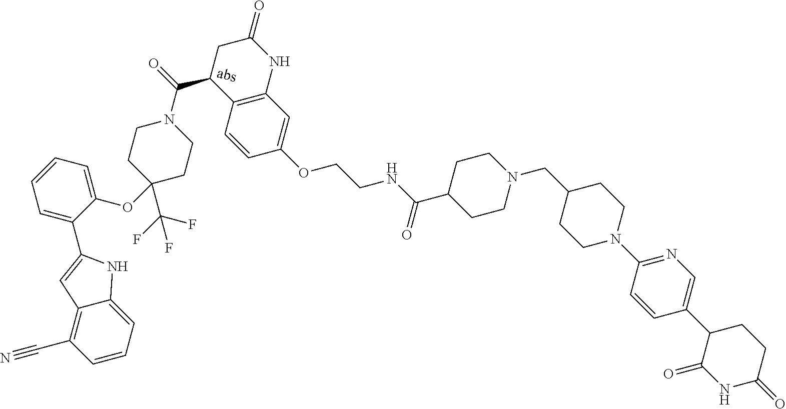 [SMILES: N#Cc1cccc2[nH]c(-c3ccccc3OC3(C(F)(F)F)CCN(C(=O)[C@H]4CC(=O)Nc5cc(OCCNC(=O)C6CCN(CC7CCN(c8ccc(C9CCC(=O)NC9=O)cn8)CC7)CC6)ccc54)CC3)cc12] |
| 209 | 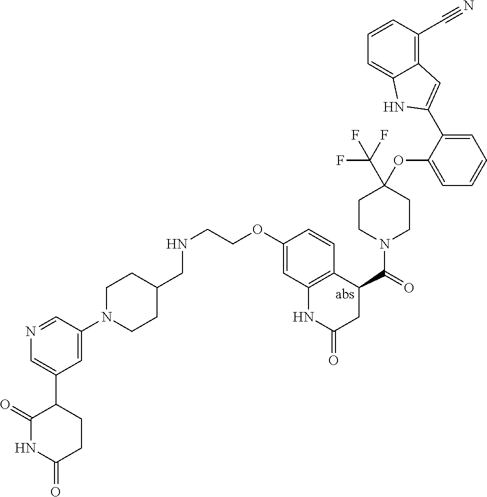 [SMILES: N#Cc1cccc2[nH]c(-c3ccccc3OC3(C(F)(F)F)CCN(C(=O)[C@H]4CC(=O)Nc5cc(OCCNCC6CCN(c7cncc(C8CCC(=O)NC8=O)c7)CC6)ccc54)CC3)cc12] |
| 210 | 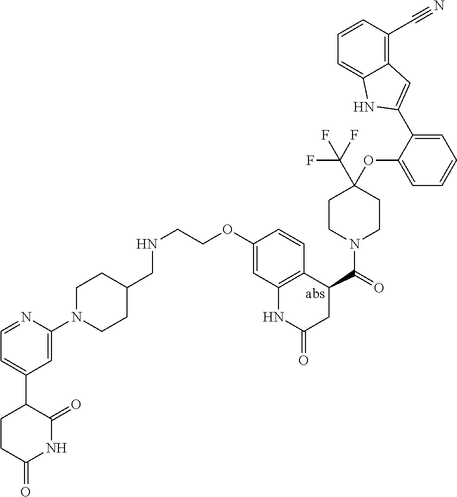 [SMILES: N#Cc1cccc2[nH]c(-c3ccccc3OC3(C(F)(F)F)CCN(C(=O)[C@H]4CC(=O)Nc5cc(OCCNCC6CCN(c7cc(C8CCC(=O)NC8=O)ccn7)CC6)ccc54)CC3)cc12] |
| 211 | 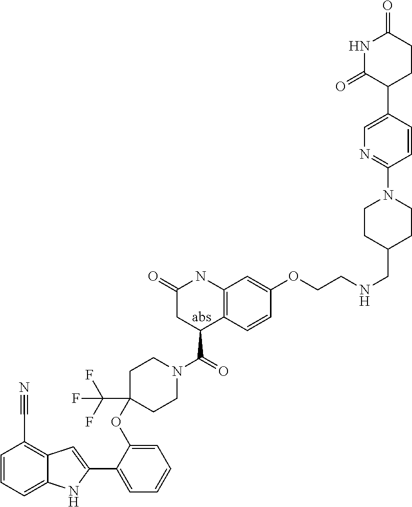 [SMILES: N#Cc1cccc2[nH]c(-c3ccccc3OC3(C(F)(F)F)CCN(C(=O)[C@H]4CC(=O)Nc5cc(OCCNCC6CCN(c7ccc(C8CCC(=O)NC8=O)cn7)CC6)ccc54)CC3)cc12] |
| 212 | 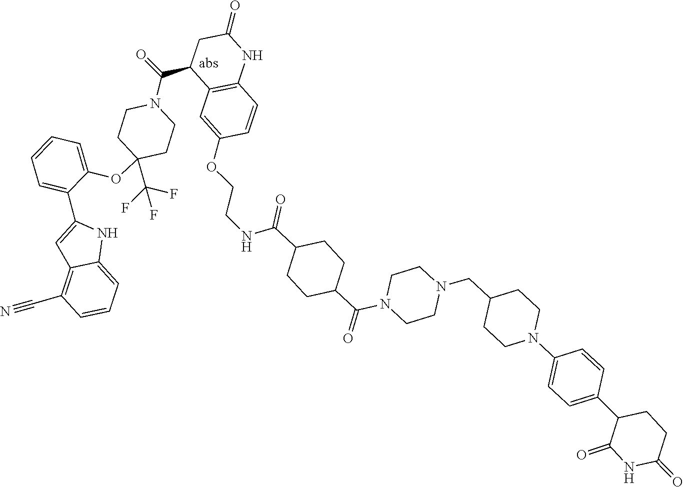 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cc9)CC8)CC7)CC6)cc54)CC3)cc12] |
| 213 | 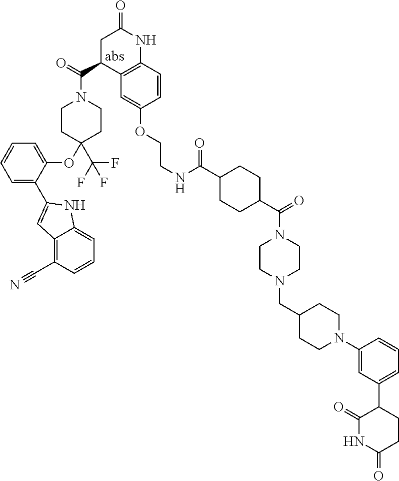 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cccc(C%10CCC(=O)NC%10=O)c9)CC8)CC7)CC6)cc54)CC3)cc12] |
| 214 | 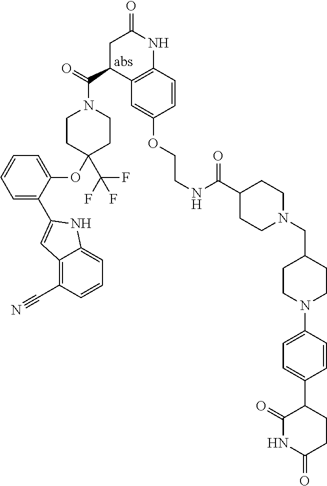 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cc8)CC7)CC6)cc54)CC3)cc12] |
| 215 | 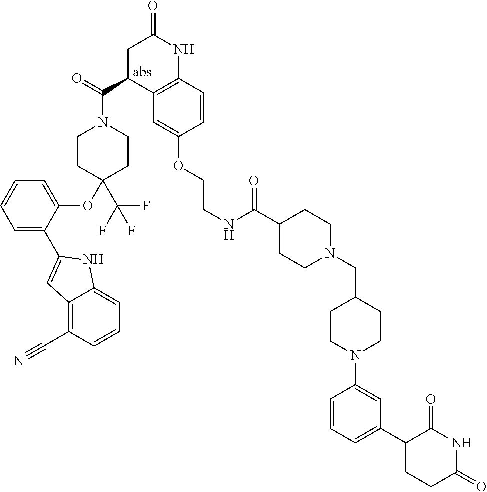 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cccc(C9CCC(=O)NC9=O)c8)CC7)CC6)cc54)CC3)cc12] |
| 216 | 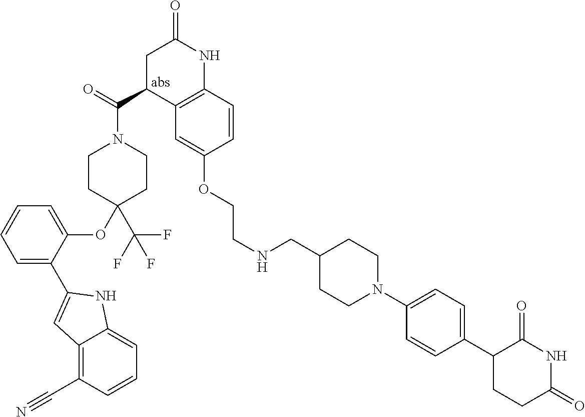 [SMILES: N#Cc1cccc2[nH]c(-c3ccccc3OC3(C(F)(F)F)CCN(C(=O)[C@H]4CC(=O)Nc5ccc(OCCNCC6CCN(c7ccc(C8CCC(=O)NC8=O)cc7)CC6)cc54)CC3)cc12] |
| 217 | 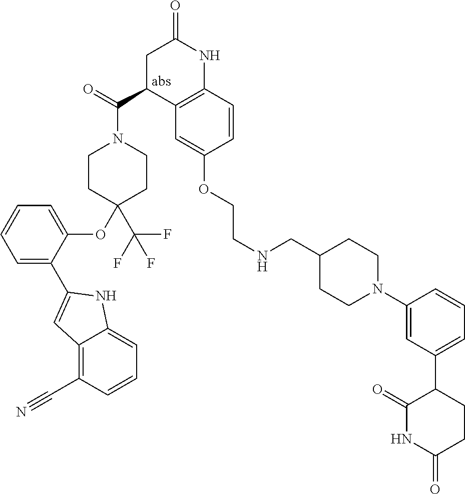 [SMILES: N#Cc1cccc2[nH]c(-c3ccccc3OC3(C(F)(F)F)CCN(C(=O)[C@H]4CC(=O)Nc5ccc(OCCNCC6CCN(c7cccc(C8CCC(=O)NC8=O)c7)CC6)cc54)CC3)cc12] |
| 218 | 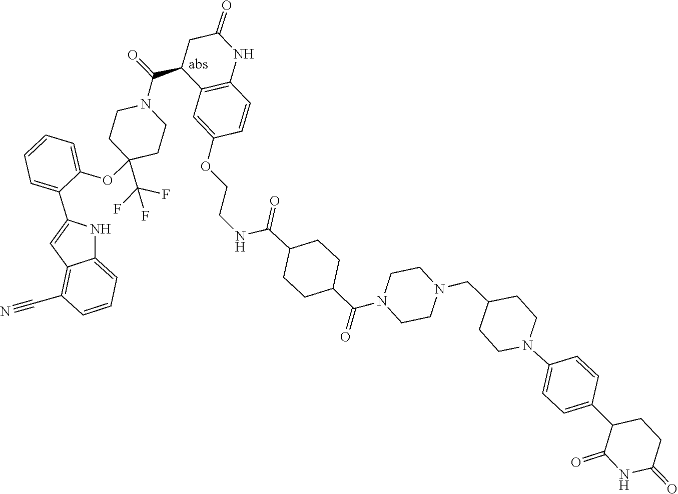 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9ccc(C%10CCC(=O)NC%10=O)cc9)CC8)CC7)CC6)cc54)CC3)cc12] |
| 219 | 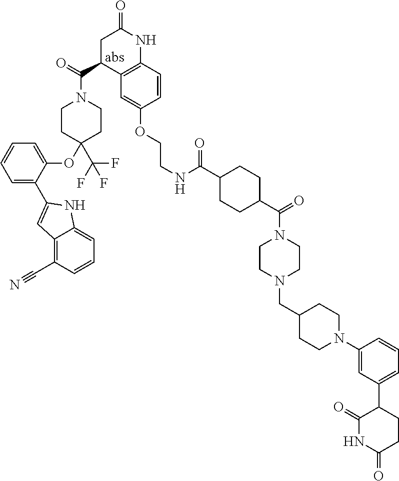 [SMILES: N#Cc1cccc2[nH]c(-c3ccccc3OC3(C(F)(F)F)CCN(C(=O)[C@H]4CC(=O)Nc5ccc(OCCNC(=O)C6CCC(C(=O)N7CCN(CC8CCN(c9cccc(C%10CCC(=O)NC%10=O)c9)CC8)CC7)CC6)cc54)CC3)cc12] |
| 220 | 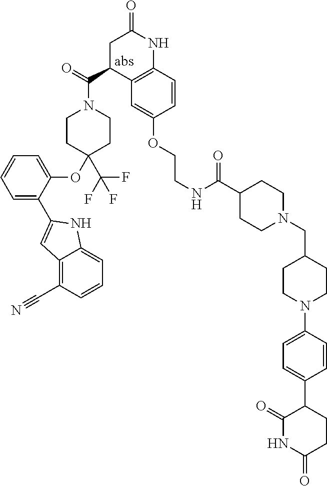 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8ccc(C9CCC(=O)NC9=O)cc8)CC7)CC6)cc54)CC3)cc12] |
| 221 | 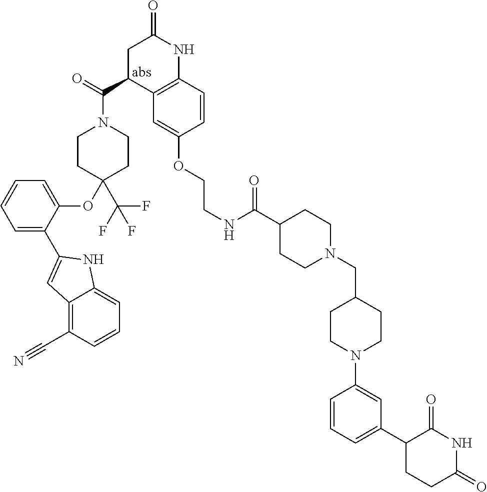 [SMILES: N#Cc1cccc2[nH]c(-c3ccccc3OC3(C(F)(F)F)CCN(C(=O)[C@H]4CC(=O)Nc5ccc(OCCNC(=O)C6CCN(CC7CCN(c8cccc(C9CCC(=O)NC9=O)c8)CC7)CC6)cc54)CC3)cc12] |
| 222 | 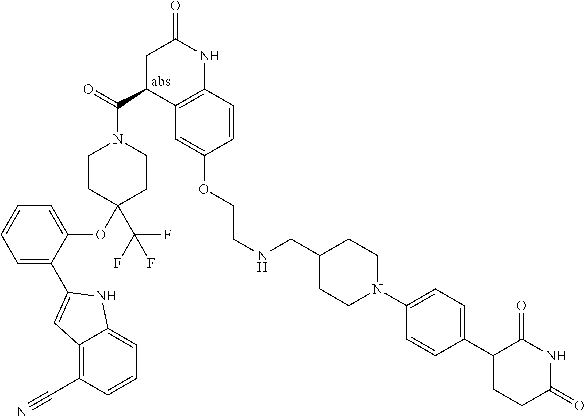 [SMILES: N#Cc1cccc2[nH]c(-c3ccccc3OC3(C(F)(F)F)CCN(C(=O)[C@H]4CC(=O)Nc5ccc(OCCNCC6CCN(c7ccc(C8CCC(=O)NC8=O)cc7)CC6)cc54)CC3)cc12] |
| 223 | 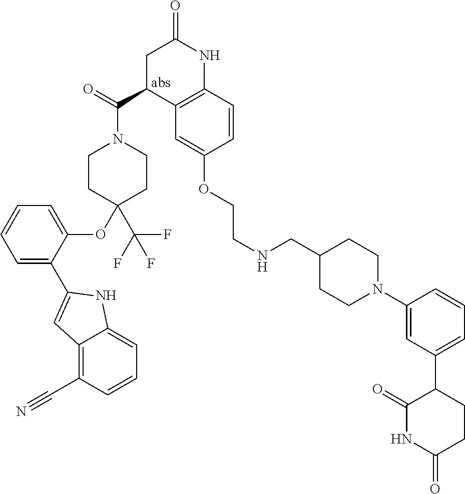 [SMILES: N#Cc1cccc2[nH]c(-c3ccccc3OC3(C(F)(F)F)CCN(C(=O)[C@H]4CC(=O)Nc5ccc(OCCNCC6CCN(c7cccc(C8CCC(=O)NC8=O)c7)CC6)cc54)CC3)cc12] |
| 224 | 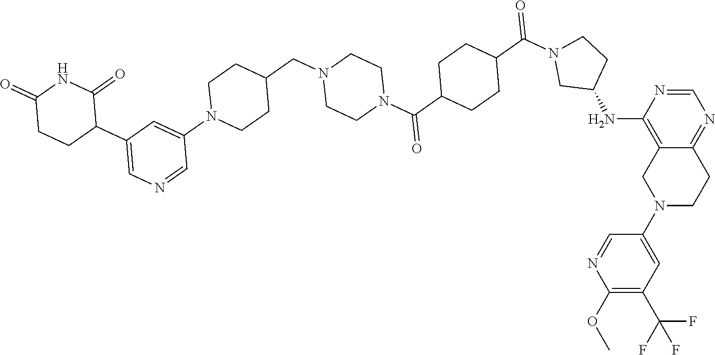 [SMILES: COc1ncc(N2CCc3ncnc(N[C@H]4CCN(C(=O)C5CCC(C(=O)N6CCN(CC7CCN(c8cncc(C9CCC(=O)NC9=O)c8)CC7)CC6)CC5)C4)c3C2)cc1C(F)(F)F] |
| 225 | 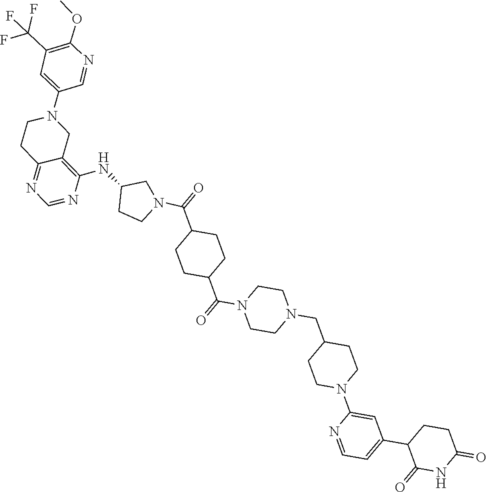 [SMILES: COc1ncc(N2CCc3ncnc(N[C@H]4CCN(C(=O)C5CCC(C(=O)N6CCN(CC7CCN(c8cc(C9CCC(=O)NC9=O)ccn8)CC7)CC6)CC5)C4)c3C2)cc1C(F)(F)F] |
| 226 | 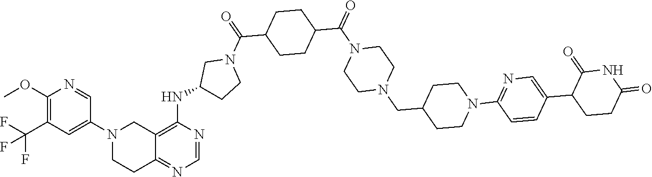 [SMILES: COc1ncc(N2CCc3ncnc(N[C@H]4CCN(C(=O)C5CCC(C(=O)N6CCN(CC7CCN(c8ccc(C9CCC(=O)NC9=O)cn8)CC7)CC6)CC5)C4)c3C2)cc1C(F)(F)F] |
| 227 | 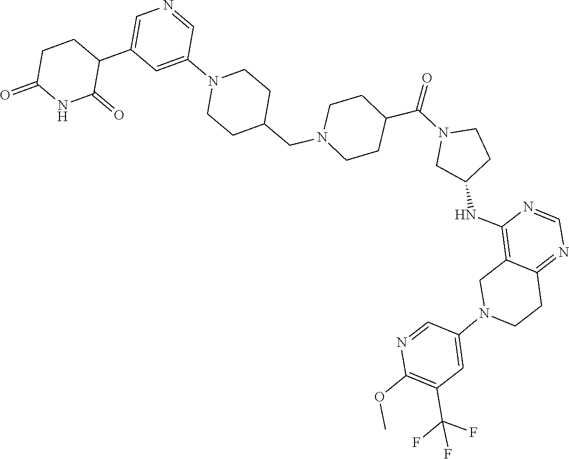 [SMILES: COc1ncc(N2CCc3ncnc(N[C@H]4CCN(C(=O)C5CCN(CC6CCN(c7cncc(C8CCC(=O)NC8=O)c7)CC6)CC5)C4)c3C2)cc1C(F)(F)F] |
| 228 | 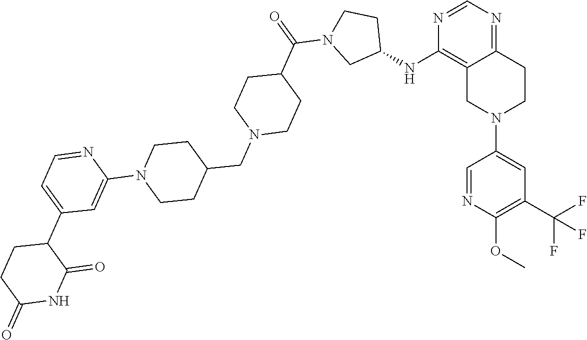 [SMILES: COc1ncc(N2CCc3ncnc(N[C@H]4CCN(C(=O)C5CCN(CC6CCN(c7cc(C8CCC(=O)NC8=O)ccn7)CC6)CC5)C4)c3C2)cc1C(F)(F)F] |
| 229 | 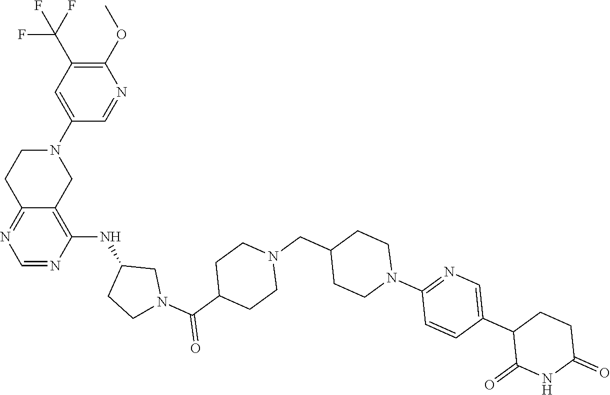 [SMILES: COc1ncc(N2CCc3ncnc(N[C@H]4CCN(C(=O)C5CCN(CC6CCN(c7ccc(C8CCC(=O)NC8=O)cn7)CC6)CC5)C4)c3C2)cc1C(F)(F)F] |
| 230 | 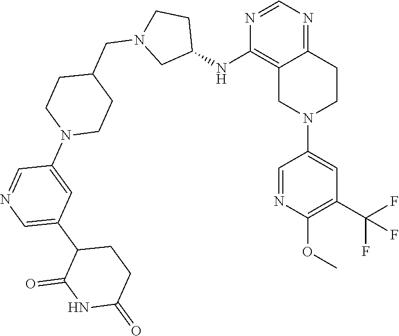 [SMILES: COc1ncc(N2CCc3ncnc(N[C@H]4CCN(CC5CCN(c6cncc(C7CCC(=O)NC7=O)c6)CC5)C4)c3C2)cc1C(F)(F)F] |
| 231 | 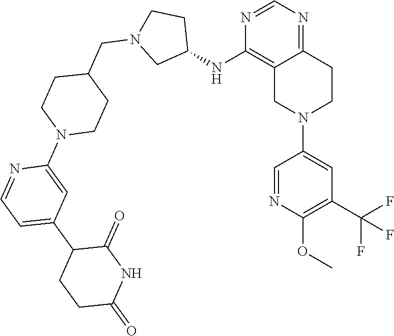 [SMILES: COc1ncc(N2CCc3ncnc(N[C@H]4CCN(CC5CCN(c6cc(C7CCC(=O)NC7=O)ccn6)CC5)C4)c3C2)cc1C(F)(F)F] |
| 232 | 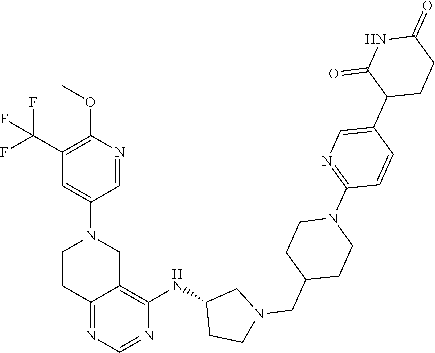 [SMILES: COc1ncc(N2CCc3ncnc(N[C@H]4CCN(CC5CCN(c6ccc(C7CCC(=O)NC7=O)cn6)CC5)C4)c3C2)cc1C(F)(F)F] |
| 233 | 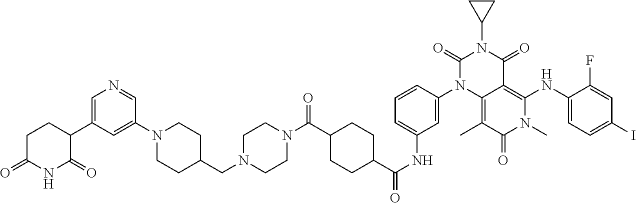 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7cncc(C8CCC(=O)NC8=O)c7)CC6)CC5)CC4)c3)c12] |
| 234 | 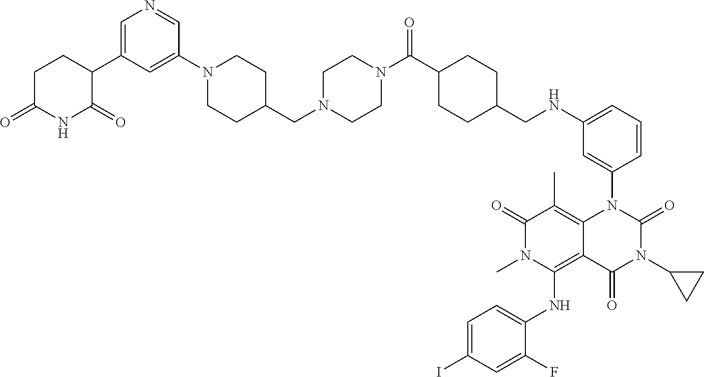 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7cncc(C8CCC(=O)NC8=O)c7)CC6)CC5)CC4)c3)c12] |
| 235 | 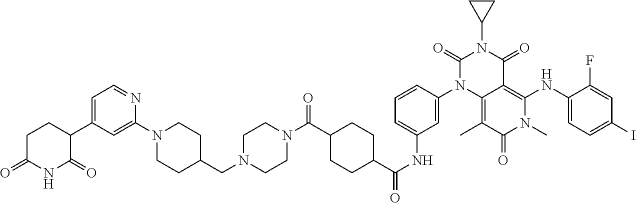 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7cc(C8CCC(=O)NC8=O)ccn7)CC6)CC5)CC4)c3)c12] |
| 236 | 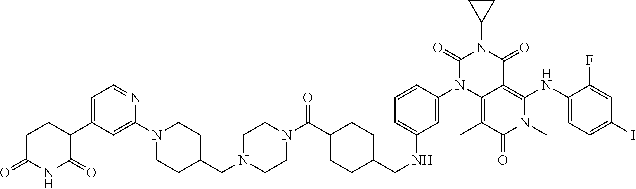 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7cc(C8CCC(=O)NC8=O)ccn7)CC6)CC5)CC4)c3)c12] |
| 237 | 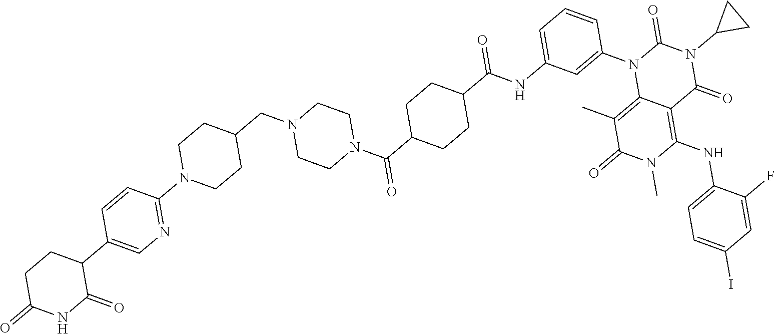 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7ccc(C8CCC(=O)NC8=O)cn7)CC6)CC5)CC4)c3)c12] |
| 238 | 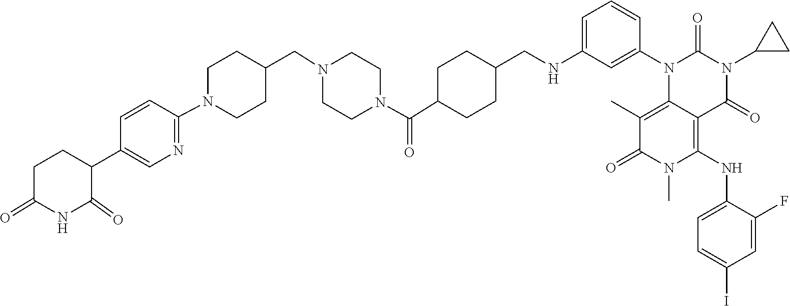 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7ccc(C8CCC(=O)NC8=O)cn7)CC6)CC5)CC4)c3)c12] |
| 239 | 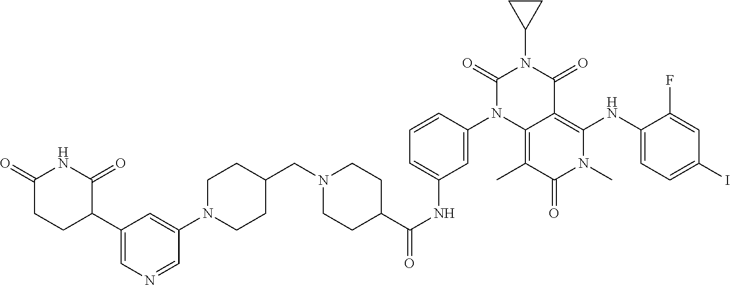 [SMILES: Cc1c(=O)n(C)c(Nc2ccc(I)cc2F)c2c(=O)n(C3CC3)c(=O)n(-c3cccc(NC(=O)C4CCN(CC5CCN(c6cncc(C7CCC(=O)NC7=O)c6)CC5)CC4)c3)c12] |
| 240 | 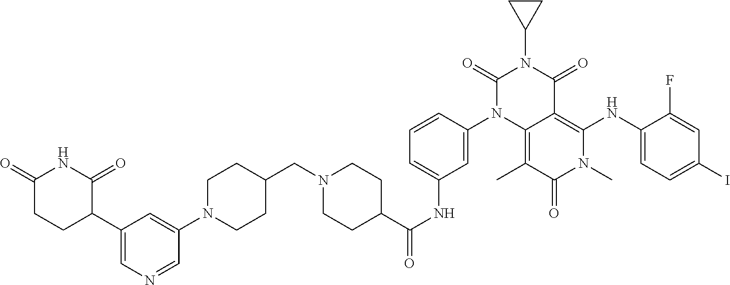 [SMILES: Cc1c(=O)n(C)c(Nc2ccc(I)cc2F)c2c(=O)n(C3CC3)c(=O)n(-c3cccc(NC(=O)C4CCN(CC5CCN(c6cncc(C7CCC(=O)NC7=O)c6)CC5)CC4)c3)c12] |
| 241 | 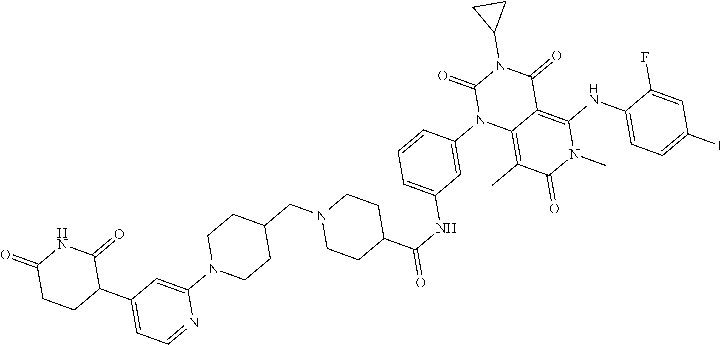 [SMILES: Cc1c(=O)n(C)c(Nc2ccc(I)cc2F)c2c(=O)n(C3CC3)c(=O)n(-c3cccc(NC(=O)C4CCN(CC5CCN(c6cc(C7CCC(=O)NC7=O)ccn6)CC5)CC4)c3)c12] |
| 242 | 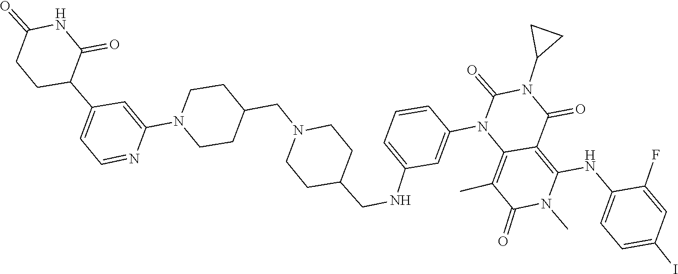 [SMILES: Cc1c(=O)n(C)c(Nc2ccc(I)cc2F)c2c(=O)n(C3CC3)c(=O)n(-c3cccc(NCC4CCN(CC5CCN(c6cc(C7CCC(=O)NC7=O)ccn6)CC5)CC4)c3)c12] |
| 243 | 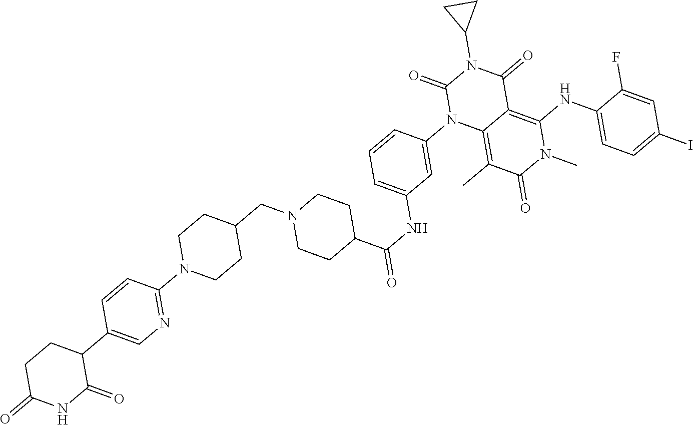 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] |
| 244 | 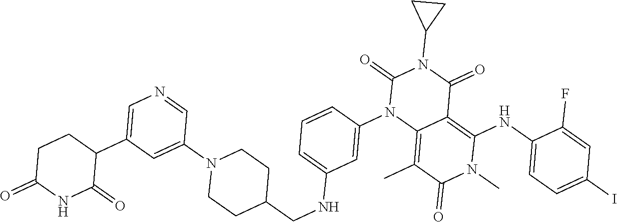 [SMILES: Cc1c(=O)n(C)c(Nc2ccc(I)cc2F)c2c(=O)n(C3CC3)c(=O)n(-c3cccc(NCC4CCN(c5cncc(C6CCC(=O)NC6=O)c5)CC4)c3)c12] |
| 245 | 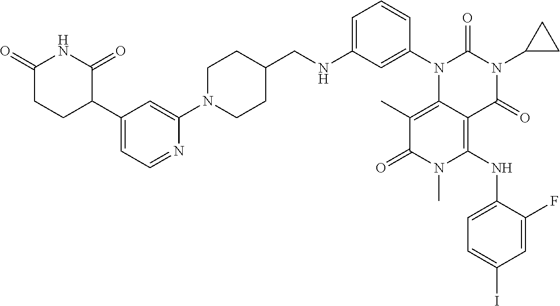 [SMILES: Cc1c(=O)n(C)c(Nc2ccc(I)cc2F)c2c(=O)n(C3CC3)c(=O)n(-c3cccc(NCC4CCN(c5cc(C6CCC(=O)NC6=O)ccn5)CC4)c3)c12] |
| 246 | 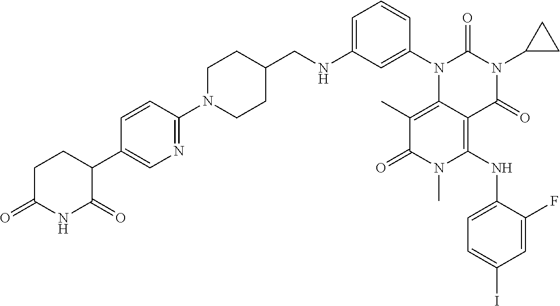 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cn5)CC4)c3)c12] |
| 247 | 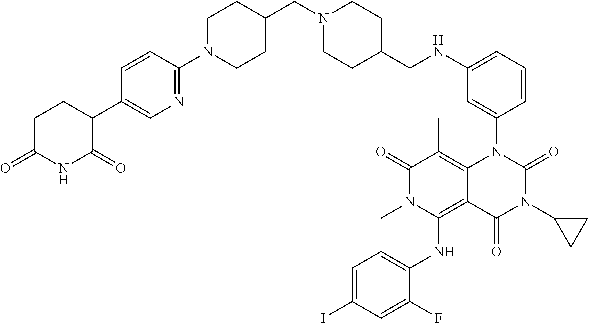 [SMILES: Cc1c(=O)n(C)c(Nc2ccc(I)cc2F)c2c(=O)n(C3CC3)c(=O)n(-c3cccc(NCC4CCN(CC5CCN(c6ccc(C7CCC(=O)NC7=O)cn6)CC5)CC4)c3)c12] |
| 248 | 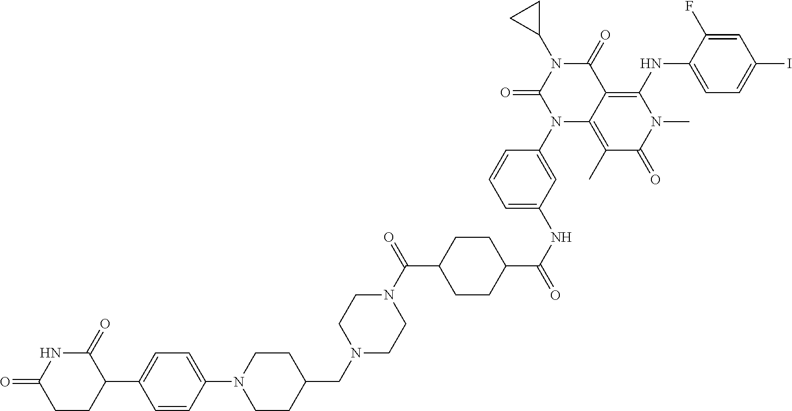 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7ccc(C8CCC(=O)NC8=O)cc7)CC6)CC5)CC4)c3)c12] |
| 249 | 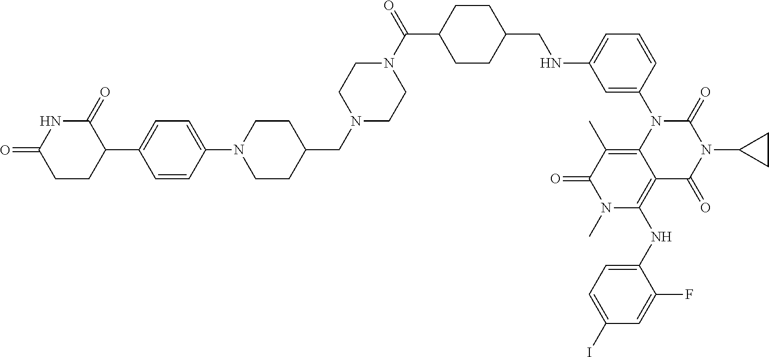 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7ccc(C8CCC(=O)NC8=O)cc7)CC6)CC5)CC4)c3)c12] |
| 250 | 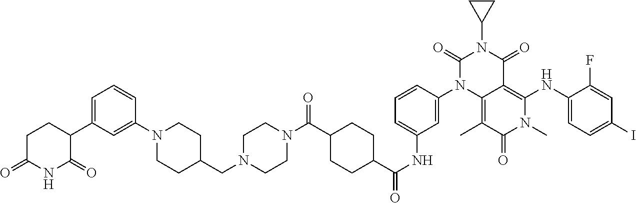 [SMILES: Cc1c(=O)n(C)c(Nc2ccc(I)cc2F)c2c(=O)n(C3CC3)c(=O)n(-c3cccc(NC(=O)C4CCC(C(=O)N5CCN(CC6CCN(c7cccc(C8CCC(=O)NC8=O)c7)CC6)CC5)CC4)c3)c12] |
| 251 | 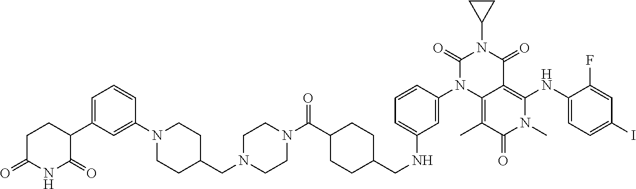 [SMILES: Cc1c(=O)n(C)c(Nc2ccc(I)cc2F)c2c(=O)n(C3CC3)c(=O)n(-c3cccc(NCC4CCC(C(=O)N5CCN(CC6CCN(c7cccc(C8CCC(=O)NC8=O)c7)CC6)CC5)CC4)c3)c12] |
| 252 | 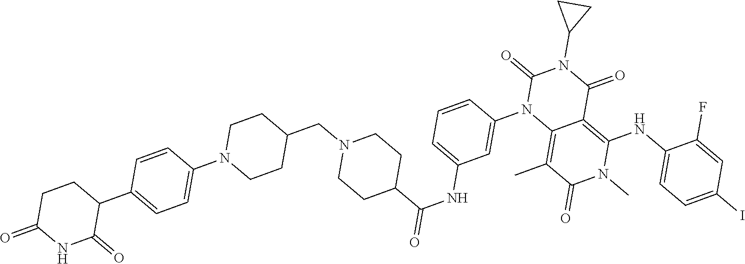 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cc6)CC5)CC4)c3)c12] |
| 253 | 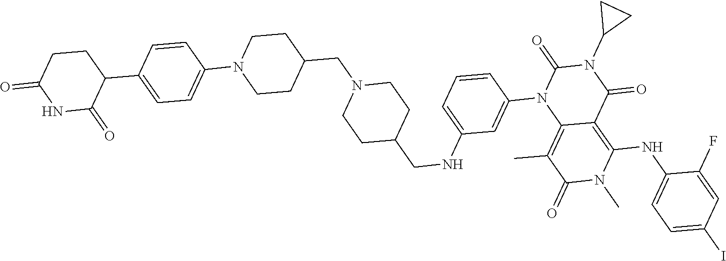 [SMILES: Cc1c(=O)n(C)c(Nc2ccc(I)cc2F)c2c(=O)n(C3CC3)c(=O)n(-c3cccc(NCC4CCN(CC5CCN(c6ccc(C7CCC(=O)NC7=O)cc6)CC5)CC4)c3)c12] |
| 254 | 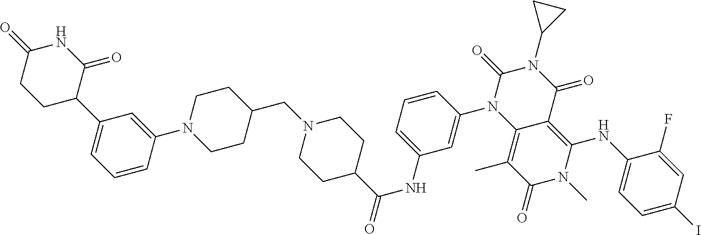 [SMILES: Cc1c(=O)n(C)c(Nc2ccc(I)cc2F)c2c(=O)n(C3CC3)c(=O)n(-c3cccc(NC(=O)C4CCN(CC5CCN(c6cccc(C7CCC(=O)NC7=O)c6)CC5)CC4)c3)c12] |
| 255 | 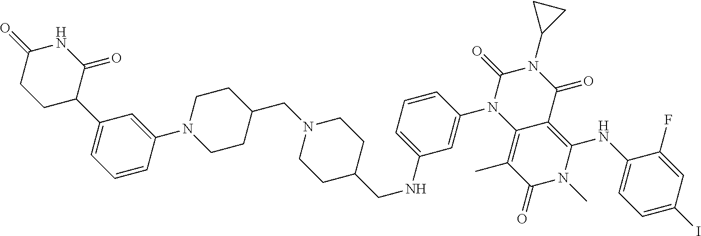 [SMILES: Cc1c(=O)n(C)c(Nc2ccc(I)cc2F)c2c(=O)n(C3CC3)c(=O)n(-c3cccc(NCC4CCN(CC5CCN(c6cccc(C7CCC(=O)NC7=O)c6)CC5)CC4)c3)c12] |
| 256 | 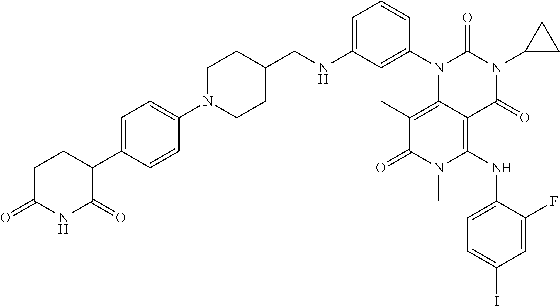 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cc5)CC4)c3)c12] |
| 257 | 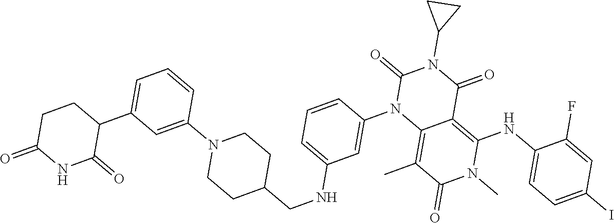 [SMILES: Cc1c(=O)n(C)c(Nc2ccc(I)cc2F)c2c(=O)n(C3CC3)c(=O)n(-c3cccc(NCC4CCN(c5cccc(C6CCC(=O)NC6=O)c5)CC4)c3)c12] |
| 258 | 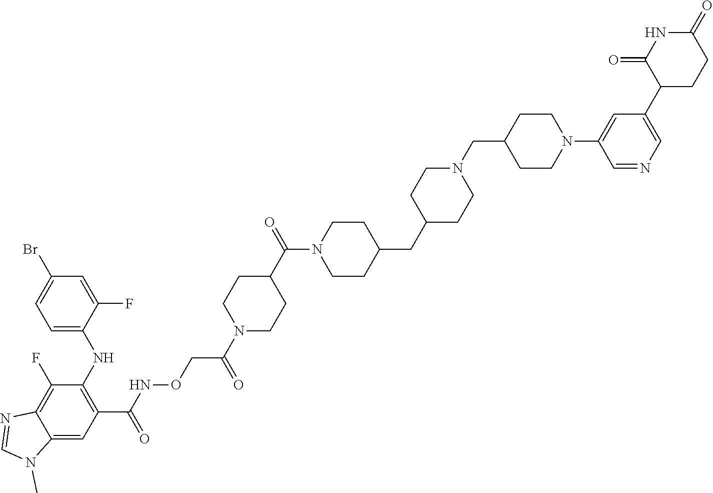 [SMILES: Cn1cnc2c(F)c(Nc3ccc(Br)cc3F)c(C(=O)NOCC(=O)N3CCC(C(=O)N4CCC(CC5CCN(CC6CCN(c7cncc(C8CCC(=O)NC8=O)c7)CC6)CC5)CC4)CC3)cc21] |
| 259 | 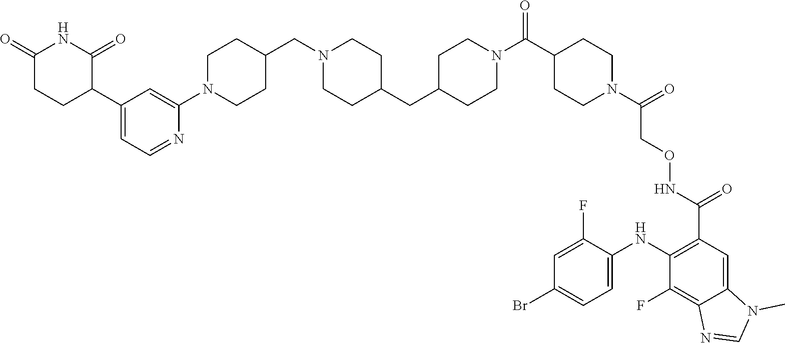 [SMILES: Cn1cnc2c(F)c(Nc3ccc(Br)cc3F)c(C(=O)NOCC(=O)N3CCC(C(=O)N4CCC(CC5CCN(CC6CCN(c7cc(C8CCC(=O)NC8=O)ccn7)CC6)CC5)CC4)CC3)cc21] |
| 260 | 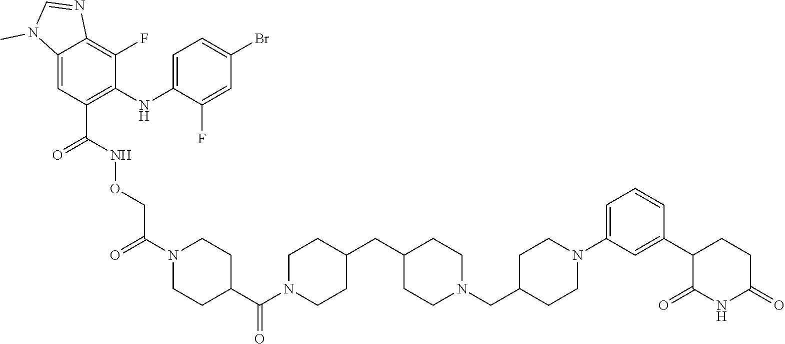 [SMILES: Cn1cnc2c(F)c(Nc3ccc(Br)cc3F)c(C(=O)NOCC(=O)N3CCC(C(=O)N4CCC(CC5CCN(CC6CCN(c7cccc(C8CCC(=O)NC8=O)c7)CC6)CC5)CC4)CC3)cc21] |
| 261 | 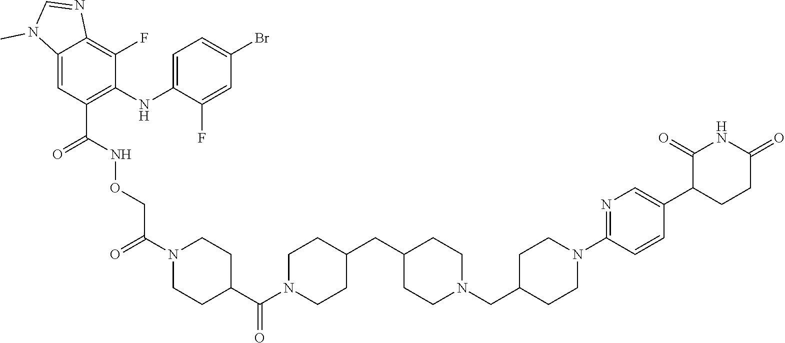 [SMILES: Cn1cnc2c(F)c(Nc3ccc(Br)cc3F)c(C(=O)NOCC(=O)N3CCC(C(=O)N4CCC(CC5CCN(CC6CCN(c7ccc(C8CCC(=O)NC8=O)cn7)CC6)CC5)CC4)CC3)cc21] |
| 262 | 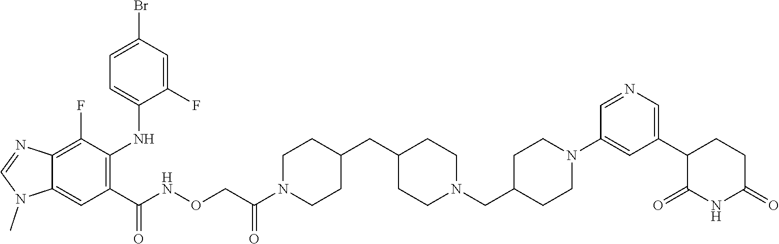 [SMILES: Cn1cnc2c(F)c(Nc3ccc(Br)cc3F)c(C(=O)NOCC(=O)N3CCC(CC4CCN(CC5CCN(c6cncc(C7CCC(=O)NC7=O)c6)CC5)CC4)CC3)cc21] |
| 263 | 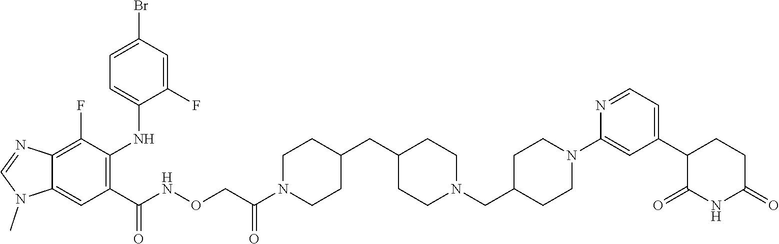 [SMILES: Cn1cnc2c(F)c(Nc3ccc(Br)cc3F)c(C(=O)NOCC(=O)N3CCC(CC4CCN(CC5CCN(c6cc(C7CCC(=O)NC7=O)ccn6)CC5)CC4)CC3)cc21] |
| 264 | 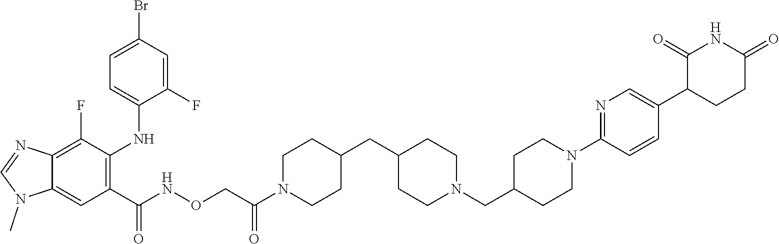 [SMILES: Cn1cnc2c(F)c(Nc3ccc(Br)cc3F)c(C(=O)NOCC(=O)N3CCC(CC4CCN(CC5CCN(c6ccc(C7CCC(=O)NC7=O)cn6)CC5)CC4)CC3)cc21] |
| 265 | 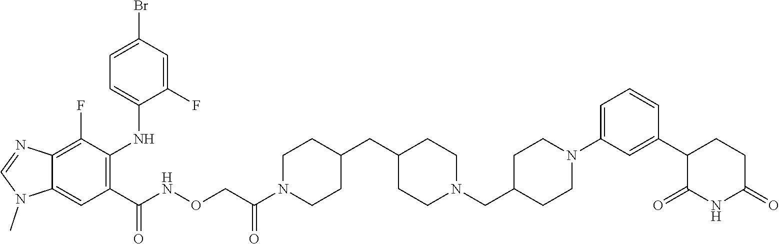 [SMILES: Cn1cnc2c(F)c(Nc3ccc(Br)cc3F)c(C(=O)NOCC(=O)N3CCC(CC4CCN(CC5CCN(c6cccc(C7CCC(=O)NC7=O)c6)CC5)CC4)CC3)cc21] |
| 266 | 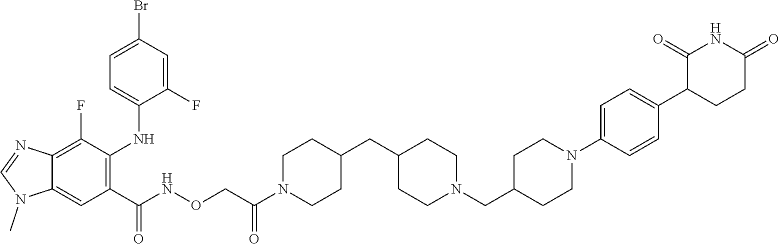 [SMILES: Cn1cnc2c(F)c(Nc3ccc(Br)cc3F)c(C(=O)NOCC(=O)N3CCC(CC4CCN(CC5CCN(c6ccc(C7CCC(=O)NC7=O)cc6)CC5)CC4)CC3)cc21] |
| 267 | 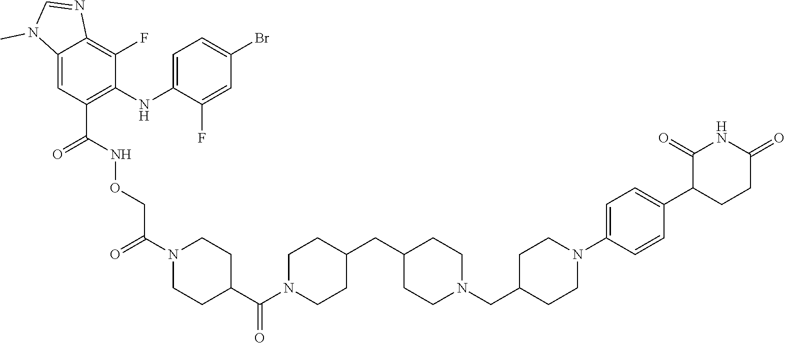 [SMILES: Cn1cnc2c(F)c(Nc3ccc(Br)cc3F)c(C(=O)NOCC(=O)N3CCC(C(=O)N4CCC(CC5CCN(CC6CCN(c7ccc(C8CCC(=O)NC8=O)cc7)CC6)CC5)CC4)CC3)cc21] |
| 268 | 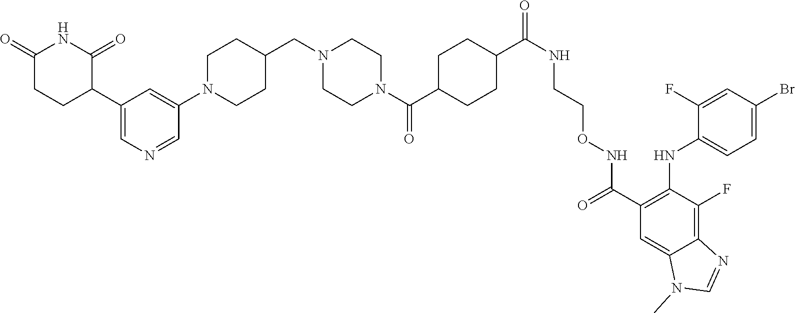 [SMILES: Cn1cnc2c(F)c(Nc3ccc(Br)cc3F)c(C(=O)NOCCNC(=O)C3CCC(C(=O)N4CCN(CC5CCN(c6cncc(C7CCC(=O)NC7=O)c6)CC5)CC4)CC3)cc21] |
| 269 | 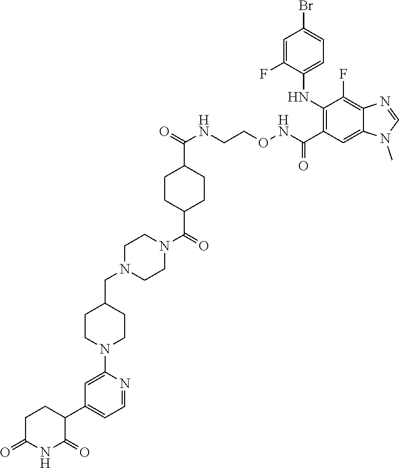 [SMILES: Cn1cnc2c(F)c(Nc3ccc(Br)cc3F)c(C(=O)NOCCNC(=O)C3CCC(C(=O)N4CCN(CC5CCN(c6cc(C7CCC(=O)NC7=O)ccn6)CC5)CC4)CC3)cc21] |
| 270 | 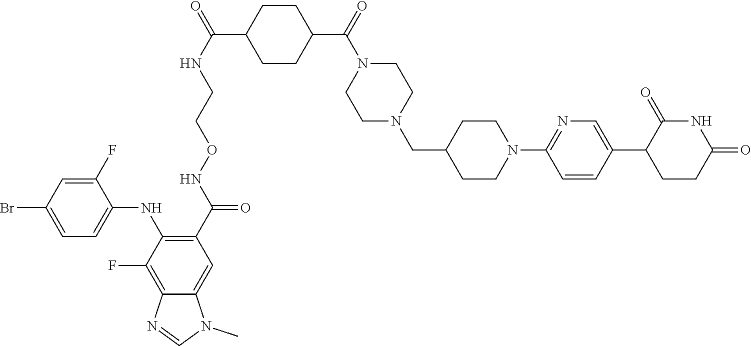 [SMILES: Cn1cnc2c(F)c(Nc3ccc(Br)cc3F)c(C(=O)NOCCNC(=O)C3CCC(C(=O)N4CCN(CC5CCN(c6ccc(C7CCC(=O)NC7=O)cn6)CC5)CC4)CC3)cc21] |
| 271 | 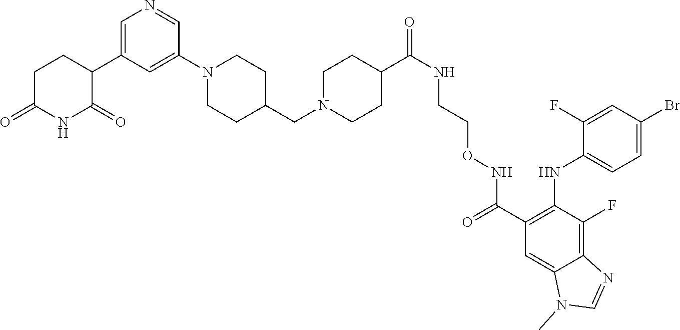 [SMILES: Cn1cnc2c(F)c(Nc3ccc(Br)cc3F)c(C(=O)NOCCNC(=O)C3CCN(CC4CCN(c5cncc(C6CCC(=O)NC6=O)c5)CC4)CC3)cc21] |
| 272 | 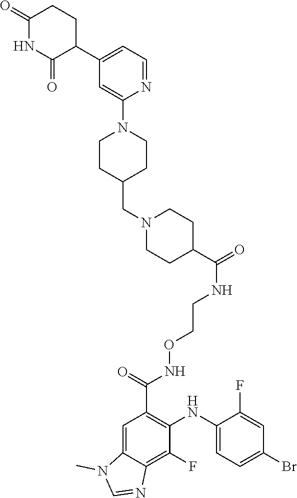 [SMILES: Cn1cnc2c(F)c(Nc3ccc(Br)cc3F)c(C(=O)NOCCNC(=O)C3CCN(CC4CCN(c5cc(C6CCC(=O)NC6=O)ccn5)CC4)CC3)cc21] |
| 273 | 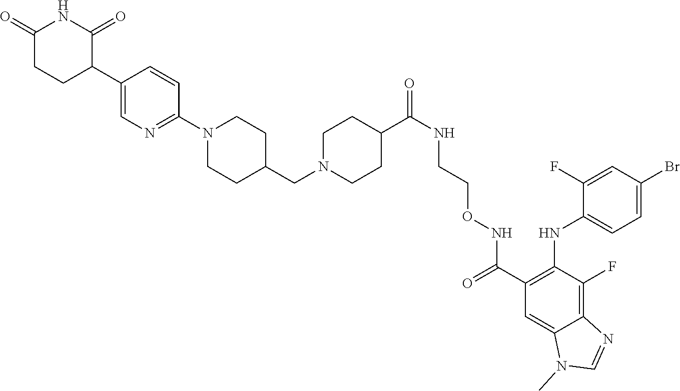 [SMILES: Cn1cnc2c(F)c(Nc3ccc(Br)cc3F)c(C(=O)NOCCNC(=O)C3CCN(CC4CCN(c5ccc(C6CCC(=O)NC6=O)cn5)CC4)CC3)cc21] |
| 274 | 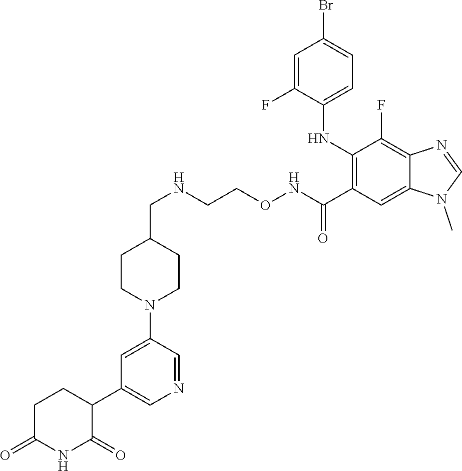 [SMILES: Cn1cnc2c(F)c(Nc3ccc(Br)cc3F)c(C(=O)NOCCNCC3CCN(c4cncc(C5CCC(=O)NC5=O)c4)CC3)cc21] |
| 275 | 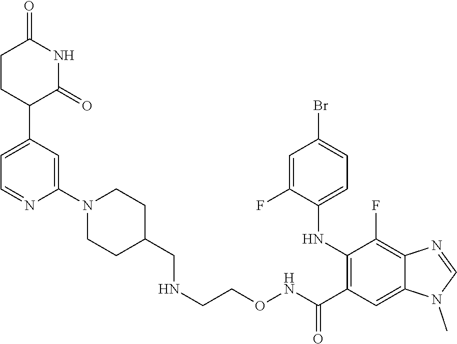 [SMILES: Cn1cnc2c(F)c(Nc3ccc(Br)cc3F)c(C(=O)NOCCNCC3CCN(c4cc(C5CCC(=O)NC5=O)ccn4)CC3)cc21] |
| 276 | 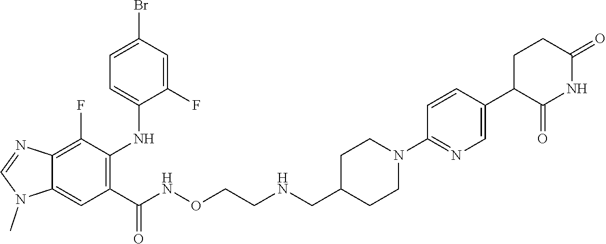 [SMILES: Cn1cnc2c(F)c(Nc3ccc(Br)cc3F)c(C(=O)NOCCNCC3CCN(c4ccc(C5CCC(=O)NC5=O)cn4)CC3)cc21] |
| 277 | 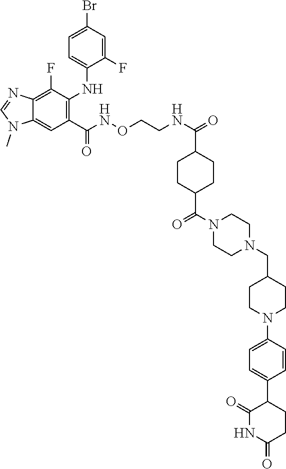 [SMILES: Cn1cnc2c(F)c(Nc3ccc(Br)cc3F)c(C(=O)NOCCNC(=O)C3CCC(C(=O)N4CCN(CC5CCN(c6ccc(C7CCC(=O)NC7=O)cc6)CC5)CC4)CC3)cc21] |
| 278 | 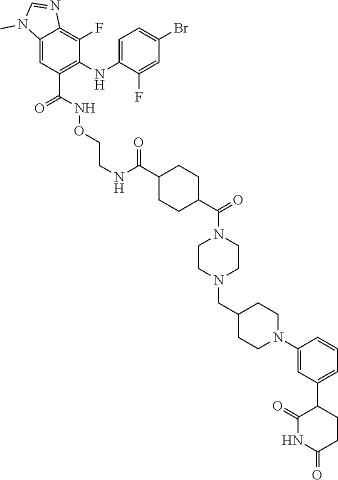 [SMILES: Cn1cnc2c(F)c(Nc3ccc(Br)cc3F)c(C(=O)NOCCNC(=O)C3CCC(C(=O)N4CCN(CC5CCN(c6cccc(C7CCC(=O)NC7=O)c6)CC5)CC4)CC3)cc21] |
| 279 | 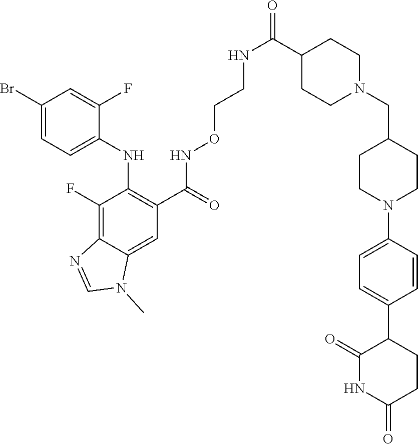 [SMILES: Cn1cnc2c(F)c(Nc3ccc(Br)cc3F)c(C(=O)NOCCNC(=O)C3CCN(CC4CCN(c5ccc(C6CCC(=O)NC6=O)cc5)CC4)CC3)cc21] |
| 280 | 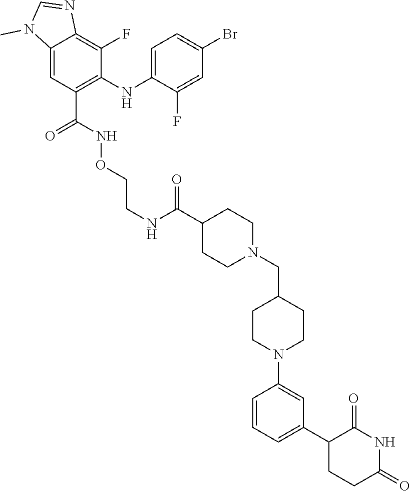 [SMILES: Cn1cnc2c(F)c(Nc3ccc(Br)cc3F)c(C(=O)NOCCNC(=O)C3CCN(CC4CCN(c5cccc(C6CCC(=O)NC6=O)c5)CC4)CC3)cc21] |
| 281 | 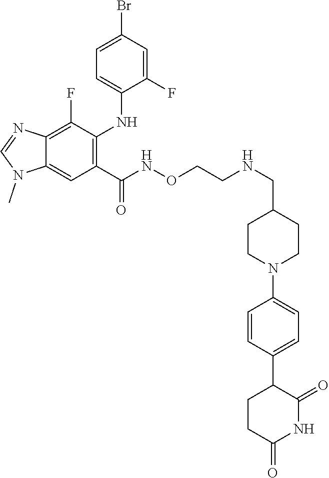 [SMILES: Cn1cnc2c(F)c(Nc3ccc(Br)cc3F)c(C(=O)NOCCNCC3CCN(c4ccc(C5CCC(=O)NC5=O)cc4)CC3)cc21] |
| 282 | 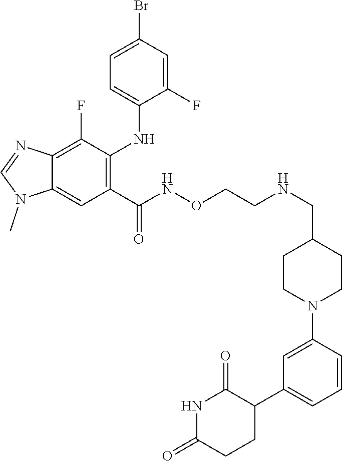 [SMILES: Cn1cnc2c(F)c(Nc3ccc(Br)cc3F)c(C(=O)NOCCNCC3CCN(c4cccc(C5CCC(=O)NC5=O)c4)CC3)cc21] |
| 283 | 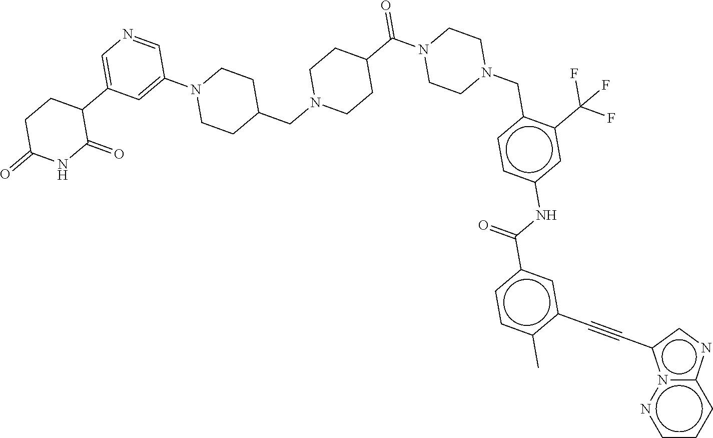 |
| 284 | 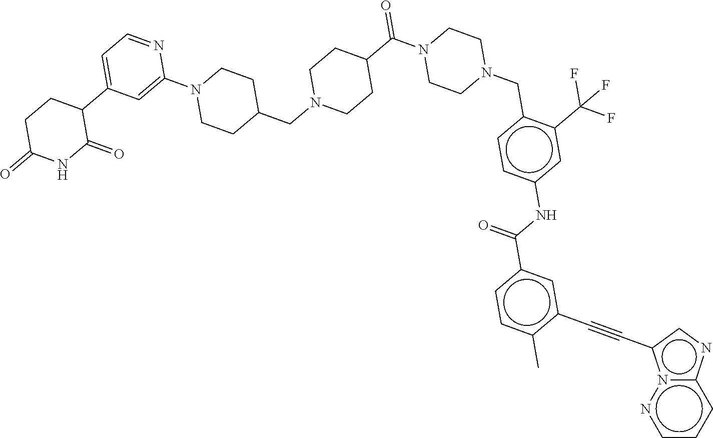 |
| 285 | 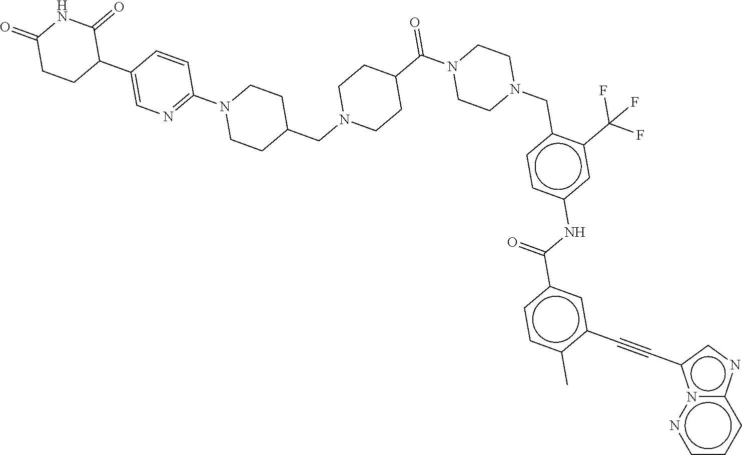 |
| 286 | 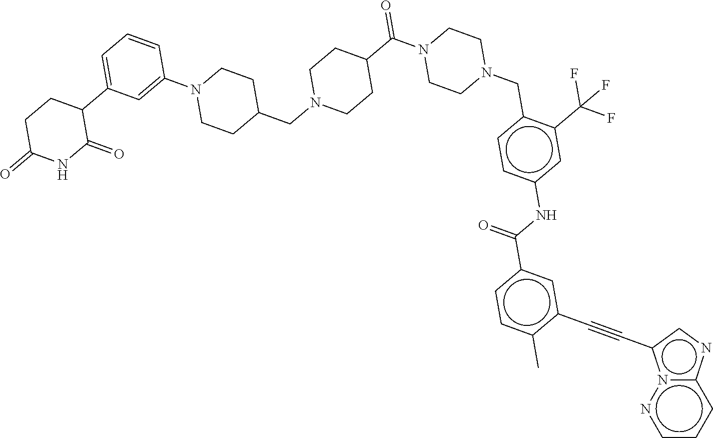 |
| 287 | 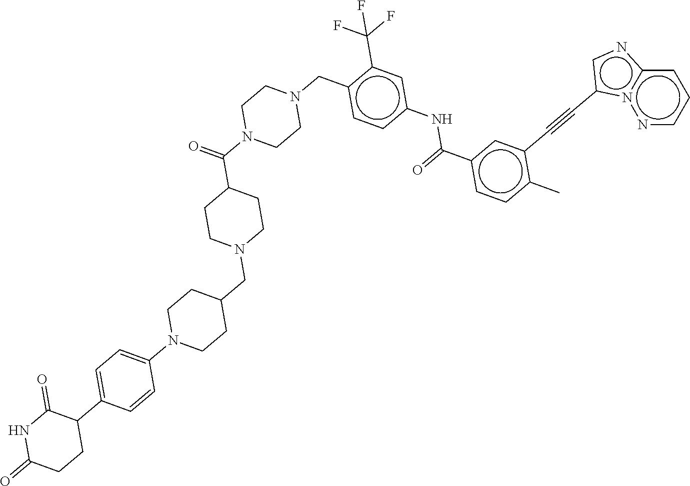 |
| 288 | 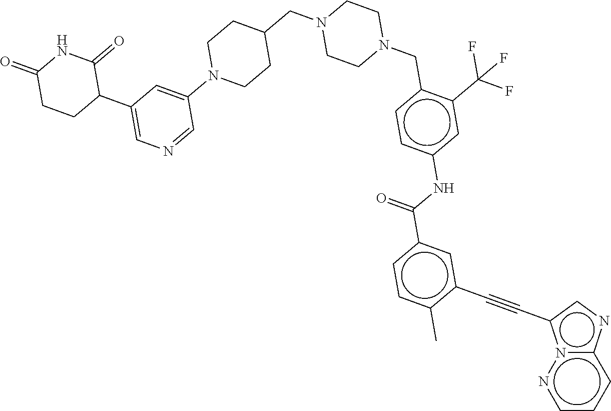 |
| 289 | 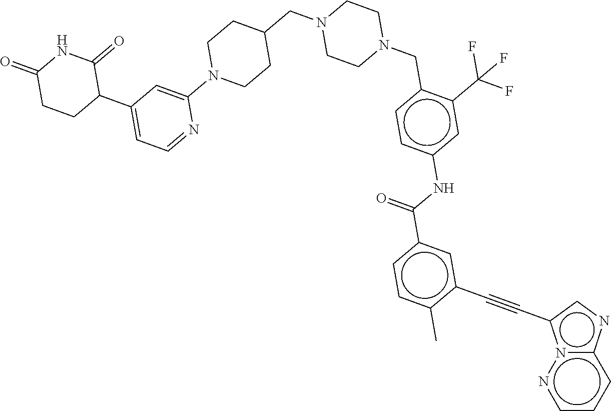 |
| 290 | 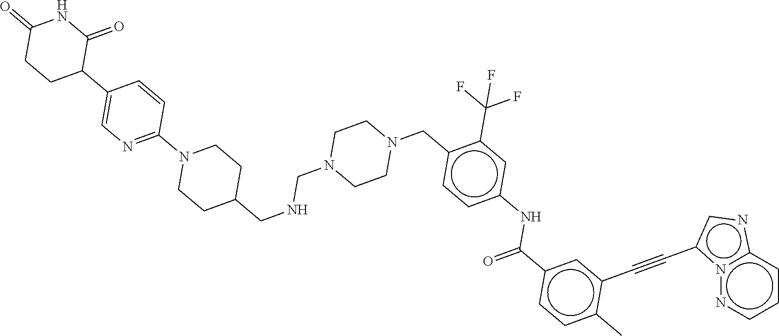 |
| 291 | 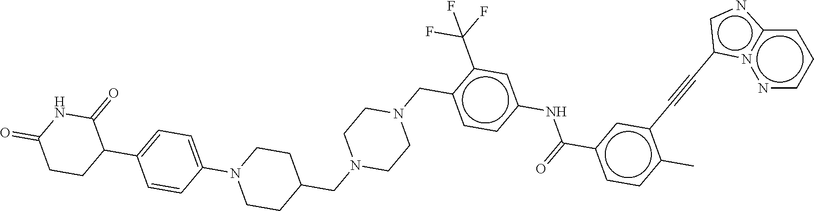 |
| 292 | 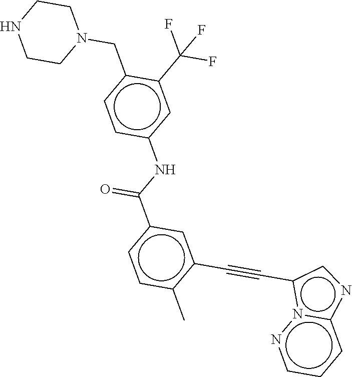 |
| 293 | 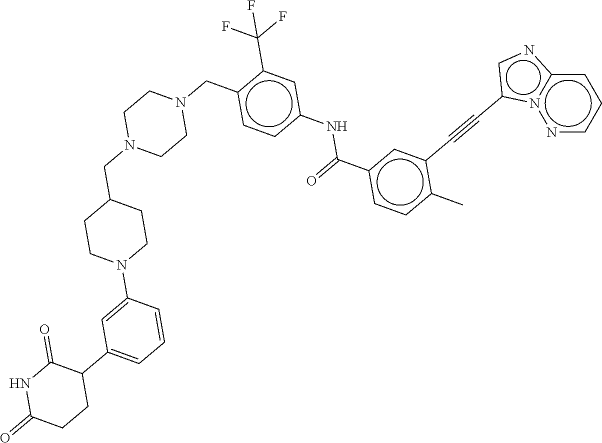 |
| 294 | 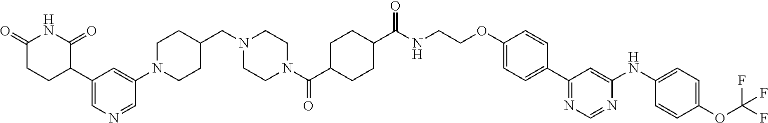 [SMILES: O=C1CCC(c2cncc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] |
| 295 | 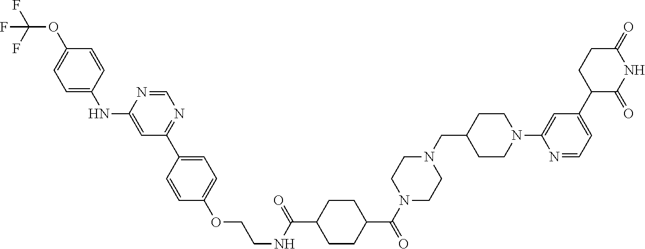 [SMILES: O=C1CCC(c2ccnc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] |
| 296 | 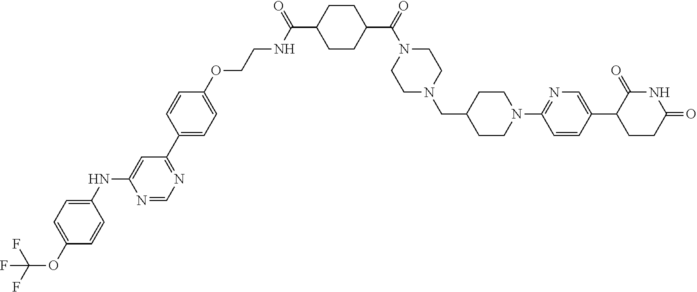 [SMILES: O=C1CCC(c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)nc2)C(=O)N1] |
| 297 | 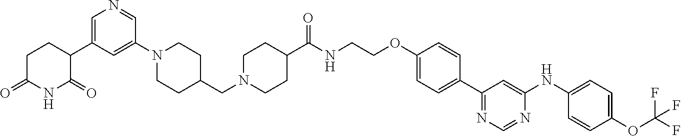 [SMILES: O=C1CCC(c2cncc(N3CCC(CN4CCC(C(=O)NCCOc5ccc(-c6cc(Nc7ccc(OC(F)(F)F)cc7)ncn6)cc5)CC4)CC3)c2)C(=O)N1] |
| 298 | 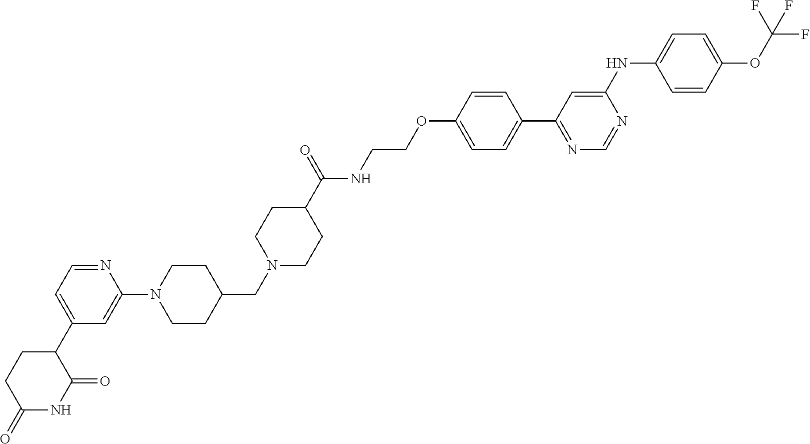 [SMILES: O=C1CCC(c2ccnc(N3CCC(CN4CCC(C(=O)NCCOc5ccc(-c6cc(Nc7ccc(OC(F)(F)F)cc7)ncn6)cc5)CC4)CC3)c2)C(=O)N1] |
| 299 | 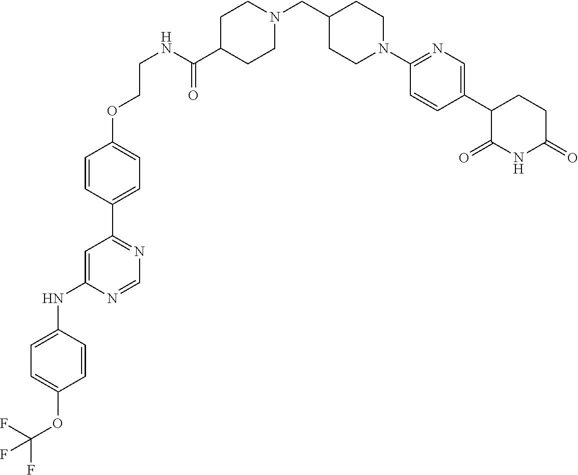 [SMILES: O=C1CCC(c2ccc(N3CCC(CN4CCC(C(=O)NCCOc5ccc(-c6cc(Nc7ccc(OC(F)(F)F)cc7)ncn6)cc5)CC4)CC3)nc2)C(=O)N1] |
| 300 | 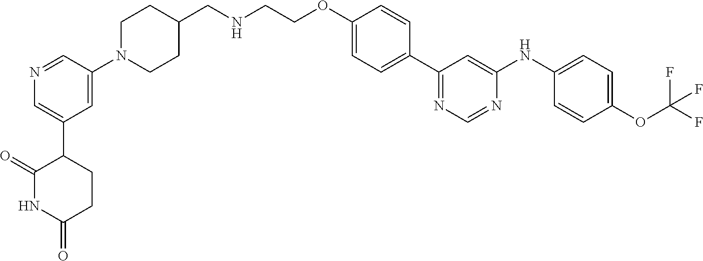 [SMILES: O=C1CCC(c2cncc(N3CCC(CNCCOc4ccc(-c5cc(Nc6ccc(OC(F)(F)F)cc6)ncn5)cc4)CC3)c2)C(=O)N1] |
| 301 | 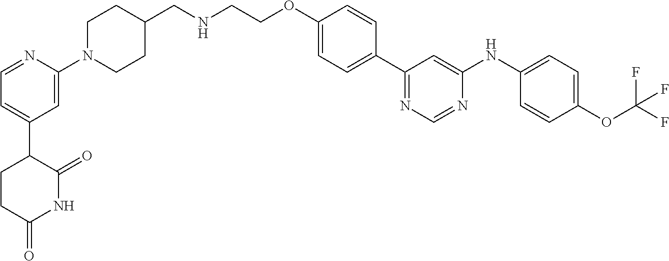 [SMILES: O=C1CCC(c2ccnc(N3CCC(CNCCOc4ccc(-c5cc(Nc6ccc(OC(F)(F)F)cc6)ncn5)cc4)CC3)c2)C(=O)N1] |
| 302 | 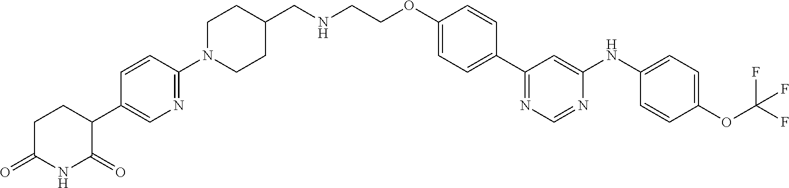 [SMILES: O=C1CCC(c2ccc(N3CCC(CNCCOc4ccc(-c5cc(Nc6ccc(OC(F)(F)F)cc6)ncn5)cc4)CC3)nc2)C(=O)N1] |
| 303 | 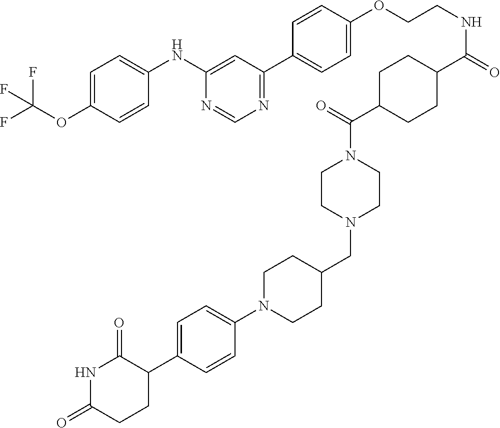 [SMILES: O=C1CCC(c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)cc2)C(=O)N1] |
| 304 | 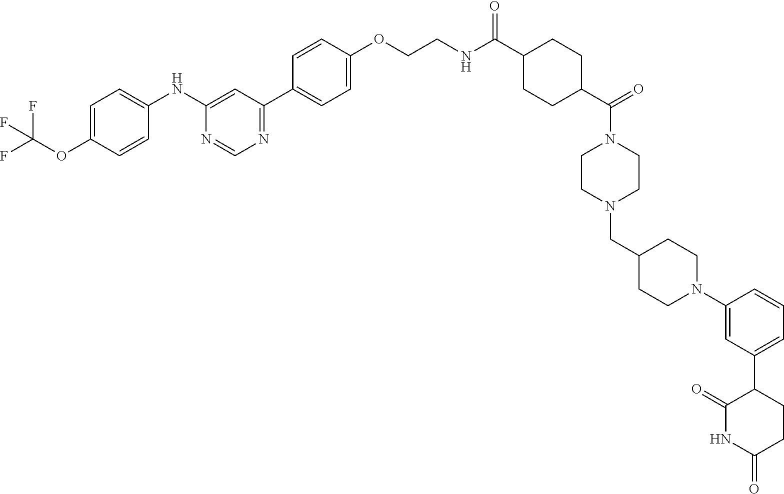 [SMILES: O=C1CCC(c2cccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] |
| 305 | 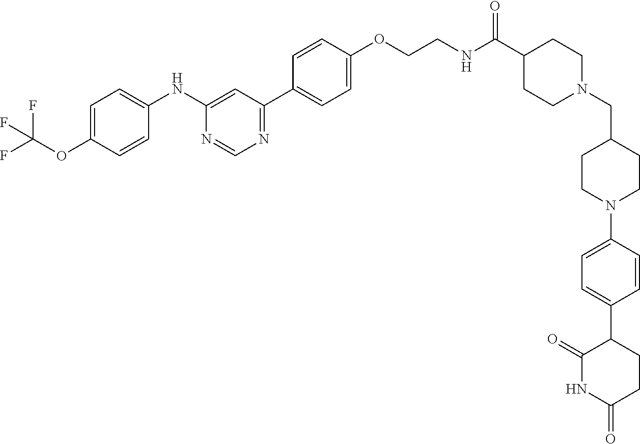 [SMILES: O=C1CCC(c2ccc(N3CCC(CN4CCC(C(=O)NCCOc5ccc(-c6cc(Nc7ccc(OC(F)(F)F)cc7)ncn6)cc5)CC4)CC3)cc2)C(=O)N1] |
| 306 | 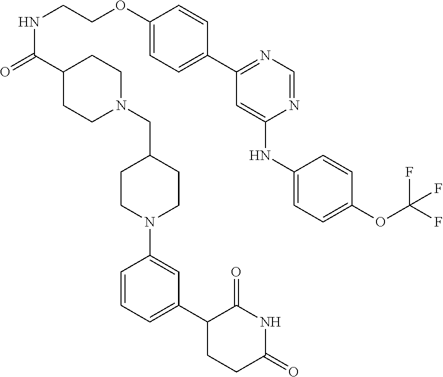 [SMILES: O=C1CCC(c2cccc(N3CCC(CN4CCC(C(=O)NCCOc5ccc(-c6cc(Nc7ccc(OC(F)(F)F)cc7)ncn6)cc5)CC4)CC3)c2)C(=O)N1] |
| 307 | 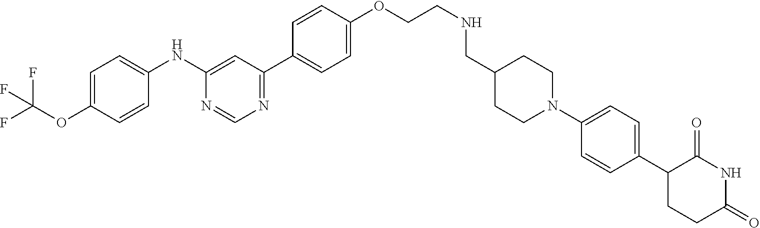 [SMILES: O=C1CCC(c2ccc(N3CCC(CNCCOc4ccc(-c5cc(Nc6ccc(OC(F)(F)F)cc6)ncn5)cc4)CC3)cc2)C(=O)N1] |
| 308 | 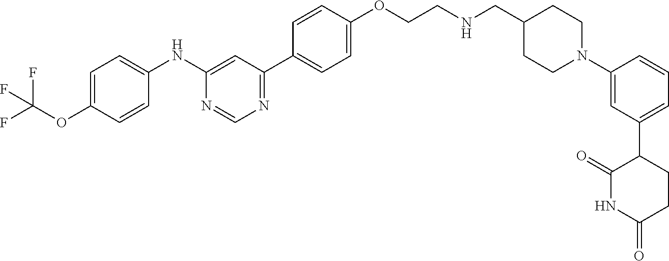 [SMILES: O=C1CCC(c2cccc(N3CCC(CNCCOc4ccc(-c5cc(Nc6ccc(OC(F)(F)F)cc6)ncn5)cc4)CC3)c2)C(=O)N1] |
| 309 | 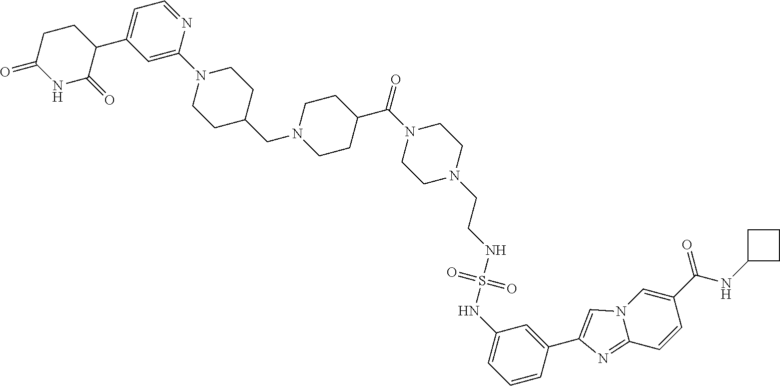 [SMILES: O=C1CCC(c2ccnc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)c2)C(=O)N1] |
| 310 | 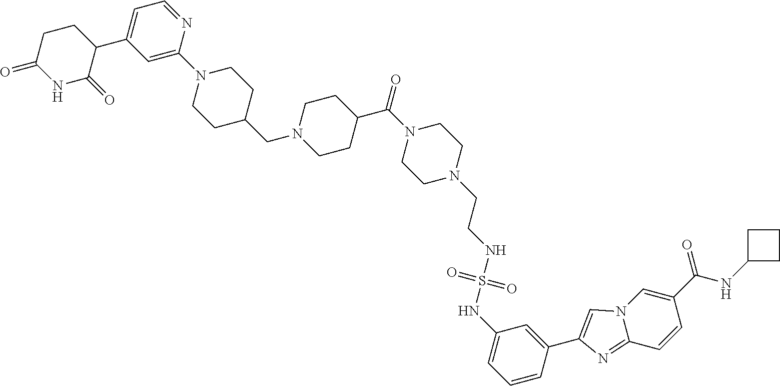 [SMILES: O=C1CCC(c2ccnc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)c2)C(=O)N1] |
| 311 | 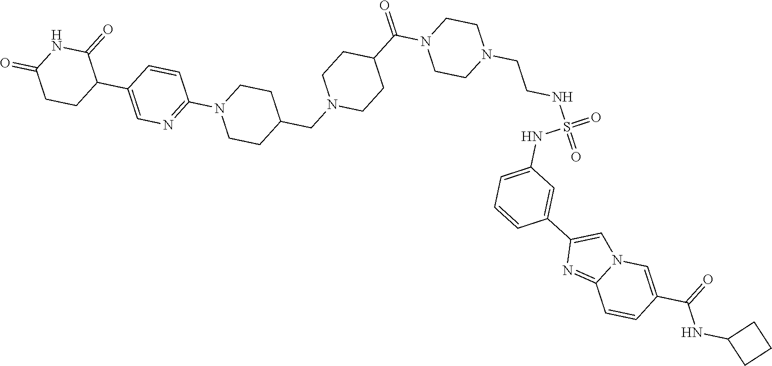 [SMILES: O=C1CCC(c2ccc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)nc2)C(=O)N1] |
| 312 | 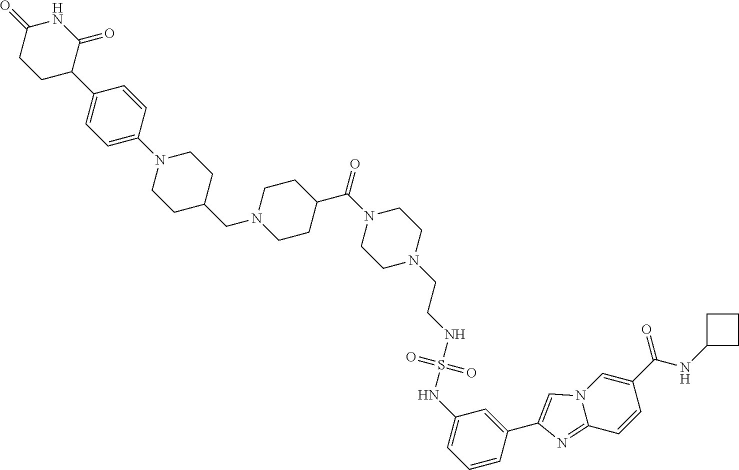 [SMILES: O=C1CCC(c2ccc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)cc2)C(=O)N1] |
| 313 | 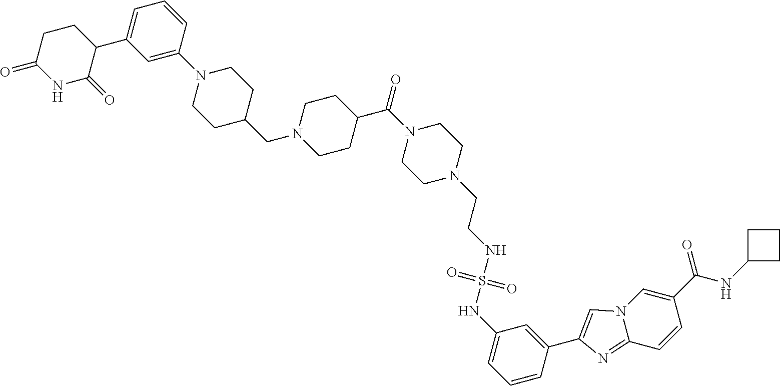 [SMILES: O=C1CCC(c2cccc(N3CCC(CN4CCC(C(=O)N5CCN(CCNS(=O)(=O)Nc6cccc(-c7cn8cc(C(=O)NC9CCC9)ccc8n7)c6)CC5)CC4)CC3)c2)C(=O)N1] |
| 314 | 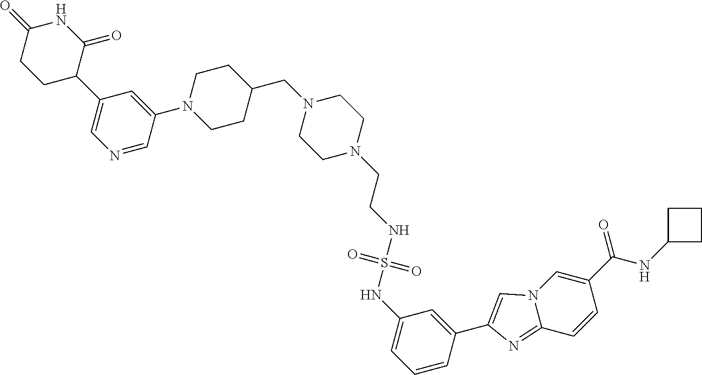 [SMILES: O=C1CCC(c2cncc(N3CCC(CN4CCN(CCNS(=O)(=O)Nc5cccc(-c6cn7cc(C(=O)NC8CCC8)ccc7n6)c5)CC4)CC3)c2)C(=O)N1] |
| 315 | 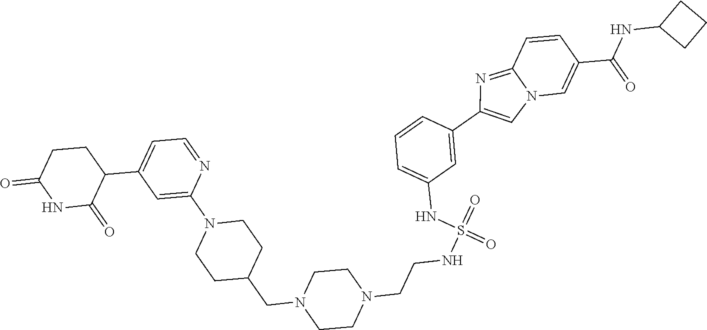 [SMILES: O=C1CCC(c2ccnc(N3CCC(CN4CCN(CCNS(=O)(=O)Nc5cccc(-c6cn7cc(C(=O)NC8CCC8)ccc7n6)c5)CC4)CC3)c2)C(=O)N1] |
| 316 | 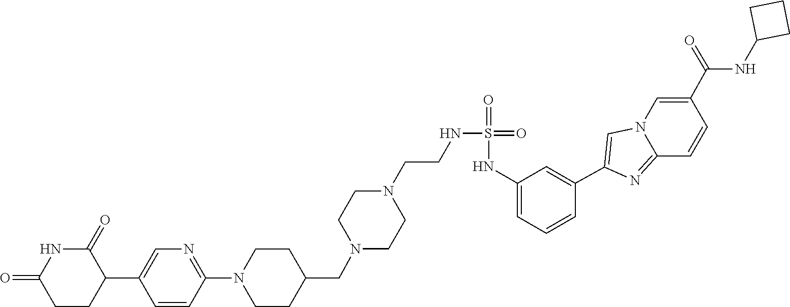 [SMILES: O=C1CCC(c2ccc(N3CCC(CN4CCN(CCNS(=O)(=O)Nc5cccc(-c6cn7cc(C(=O)NC8CCC8)ccc7n6)c5)CC4)CC3)nc2)C(=O)N1] |
| 317 | 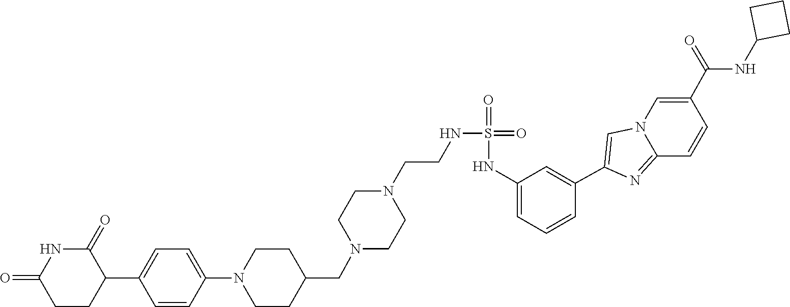 [SMILES: O=C1CCC(c2ccc(N3CCC(CN4CCN(CCNS(=O)(=O)Nc5cccc(-c6cn7cc(C(=O)NC8CCC8)ccc7n6)c5)CC4)CC3)cc2)C(=O)N1] |
| 318 | 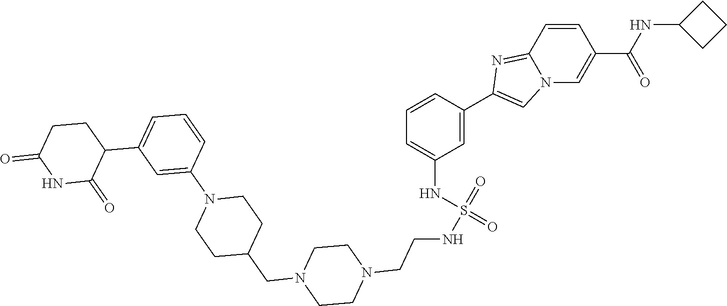 [SMILES: O=C1CCC(c2cccc(N3CCC(CN4CCN(CCNS(=O)(=O)Nc5cccc(-c6cn7cc(C(=O)NC8CCC8)ccc7n6)c5)CC4)CC3)c2)C(=O)N1] |
| 319 | 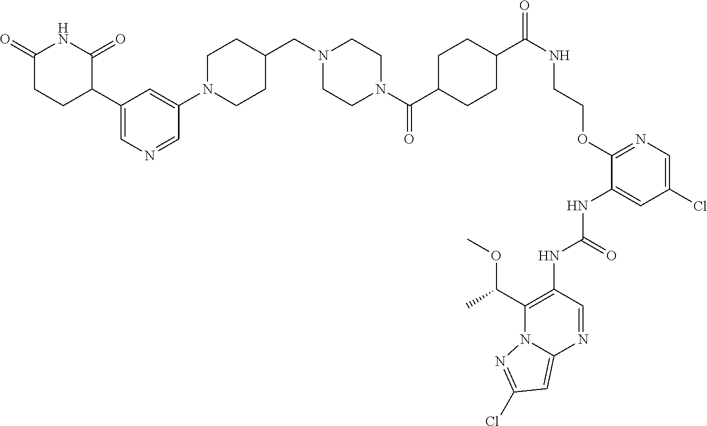 [SMILES: CO[C@@H](C)c1c(NC(=O)Nc2cc(Cl)cnc2OCCNC(=O)C2CCC(C(=O)N3CCN(CC4CCN(c5cncc(C6CCC(=O)NC6=O)c5)CC4)CC3)CC2)cnc2cc(Cl)nn12] |
| 320 | 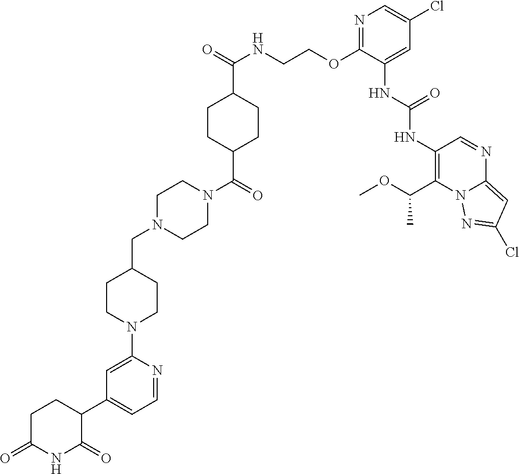 [SMILES: CO[C@@H](C)c1c(NC(=O)Nc2cc(Cl)cnc2OCCNC(=O)C2CCC(C(=O)N3CCN(CC4CCN(c5cc(C6CCC(=O)NC6=O)ccn5)CC4)CC3)CC2)cnc2cc(Cl)nn12] |
| 321 | 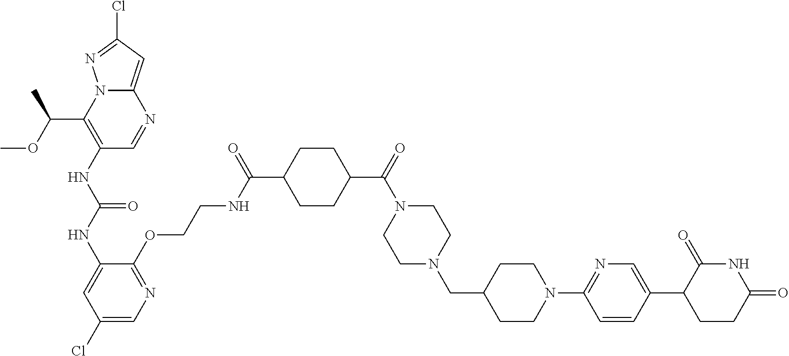 [SMILES: CO[C@@H](C)c1c(NC(=O)Nc2cc(Cl)cnc2OCCNC(=O)C2CCC(C(=O)N3CCN(CC4CCN(c5ccc(C6CCC(=O)NC6=O)cn5)CC4)CC3)CC2)cnc2cc(Cl)nn12] |
| 322 | 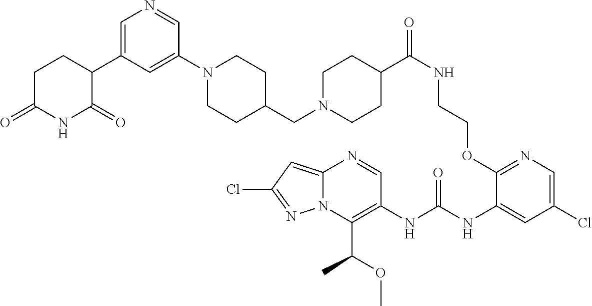 [SMILES: CO[C@@H](C)c1c(NC(=O)Nc2cc(Cl)cnc2OCCNC(=O)C2CCN(CC3CCN(c4cncc(C5CCC(=O)NC5=O)c4)CC3)CC2)cnc2cc(Cl)nn12] |
| 323 | 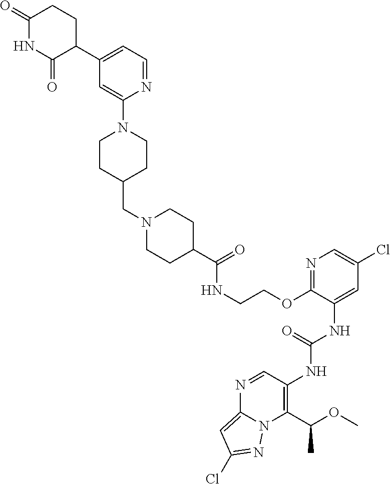 [SMILES: CO[C@@H](C)c1c(NC(=O)Nc2cc(Cl)cnc2OCCNC(=O)C2CCN(CC3CCN(c4cc(C5CCC(=O)NC5=O)ccn4)CC3)CC2)cnc2cc(Cl)nn12] |
| 324 | 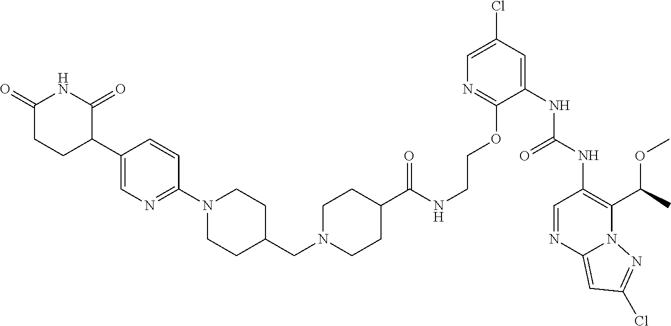 [SMILES: CO[C@@H](C)c1c(NC(=O)Nc2cc(Cl)cnc2OCCNC(=O)C2CCN(CC3CCN(c4ccc(C5CCC(=O)NC5=O)cn4)CC3)CC2)cnc2cc(Cl)nn12] |
| 325 | 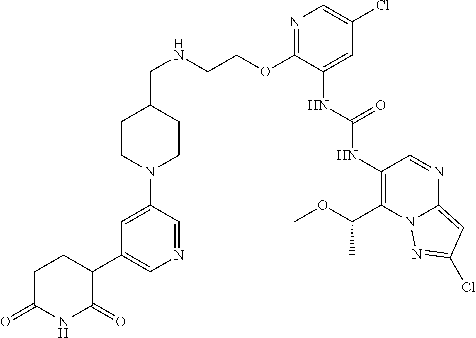 [SMILES: CO[C@@H](C)c1c(NC(=O)Nc2cc(Cl)cnc2OCCNCC2CCN(c3cncc(C4CCC(=O)NC4=O)c3)CC2)cnc2cc(Cl)nn12] |
| 326 | 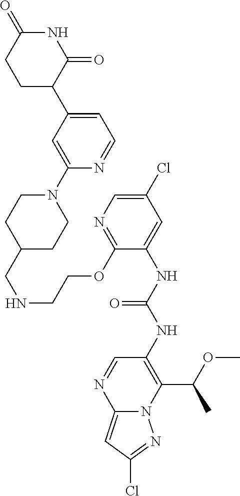 [SMILES: CO[C@@H](C)c1c(NC(=O)Nc2cc(Cl)cnc2OCCNCC2CCN(c3cc(C4CCC(=O)NC4=O)ccn3)CC2)cnc2cc(Cl)nn12] |
| 327 | 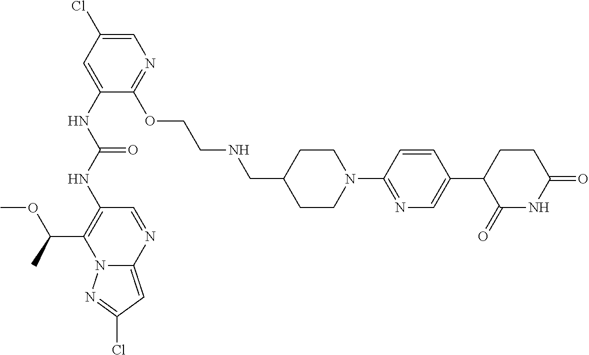 [SMILES: CO[C@H](C)c1c(NC(=O)Nc2cc(Cl)cnc2OCCNCC2CCN(c3ccc(C4CCC(=O)NC4=O)cn3)CC2)cnc2cc(Cl)nn12] |
| 328 | 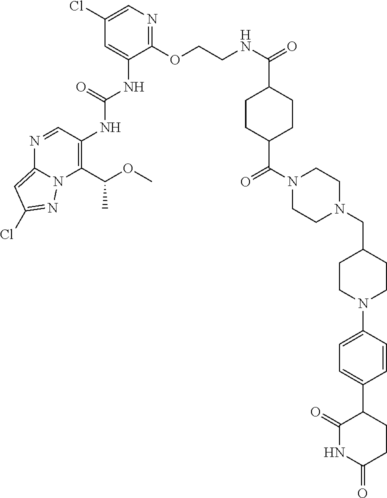 [SMILES: CO[C@H](C)c1c(NC(=O)Nc2cc(Cl)cnc2OCCNC(=O)C2CCC(C(=O)N3CCN(CC4CCN(c5ccc(C6CCC(=O)NC6=O)cc5)CC4)CC3)CC2)cnc2cc(Cl)nn12] |
| 329 | 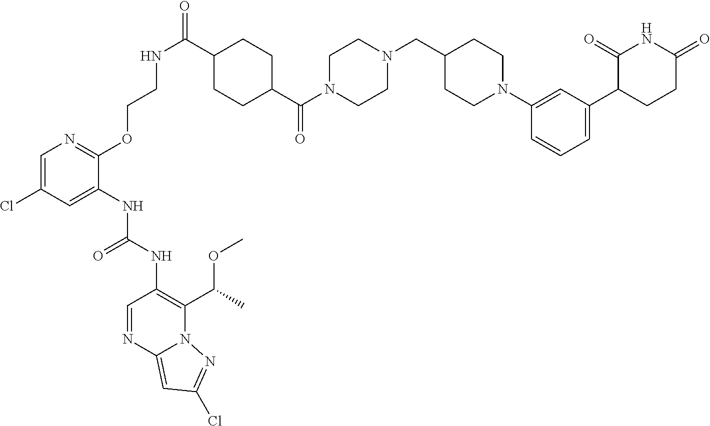 [SMILES: CO[C@H](C)c1c(NC(=O)Nc2cc(Cl)cnc2OCCNC(=O)C2CCC(C(=O)N3CCN(CC4CCN(c5cccc(C6CCC(=O)NC6=O)c5)CC4)CC3)CC2)cnc2cc(Cl)nn12] |
| 330 | 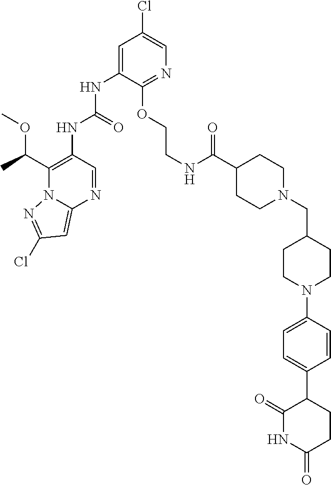 [SMILES: CO[C@H](C)c1c(NC(=O)Nc2cc(Cl)cnc2OCCNC(=O)C2CCN(CC3CCN(c4ccc(C5CCC(=O)NC5=O)cc4)CC3)CC2)cnc2cc(Cl)nn12] |
| 331 | 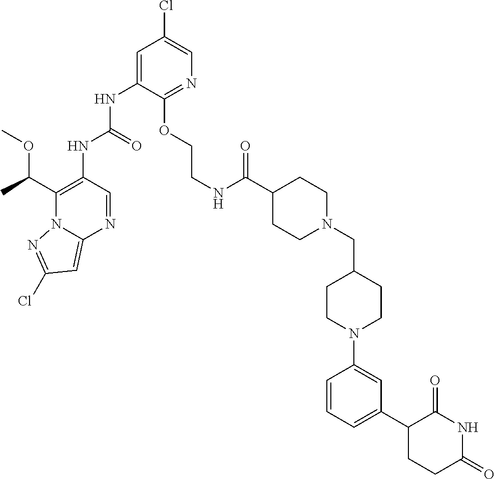 [SMILES: CO[C@H](C)c1c(NC(=O)Nc2cc(Cl)cnc2OCCNC(=O)C2CCN(CC3CCN(c4cccc(C5CCC(=O)NC5=O)c4)CC3)CC2)cnc2cc(Cl)nn12] |
| 332 | 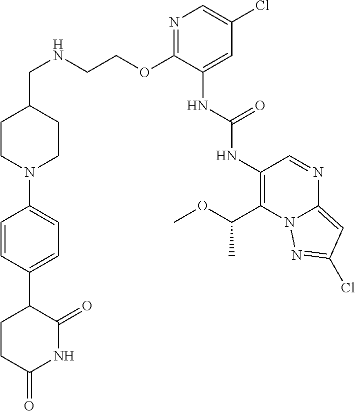 [SMILES: CO[C@@H](C)c1c(NC(=O)Nc2cc(Cl)cnc2OCCNCC2CCN(c3ccc(C4CCC(=O)NC4=O)cc3)CC2)cnc2cc(Cl)nn12] |
| 333 | 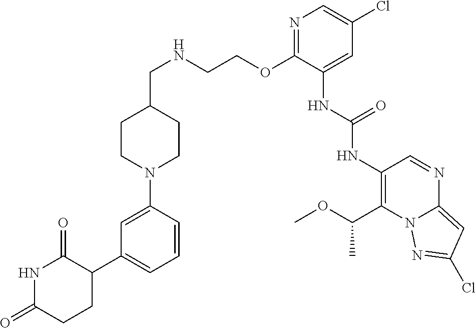 [SMILES: CO[C@@H](C)c1c(NC(=O)Nc2cc(Cl)cnc2OCCNCC2CCN(c3cccc(C4CCC(=O)NC4=O)c3)CC2)cnc2cc(Cl)nn12] |
| 334 | 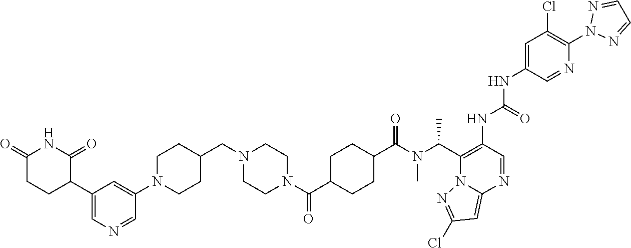 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4cncc(C5CCC(=O)NC5=O)c4)CC3)CC2)CC1] |
| 335 | 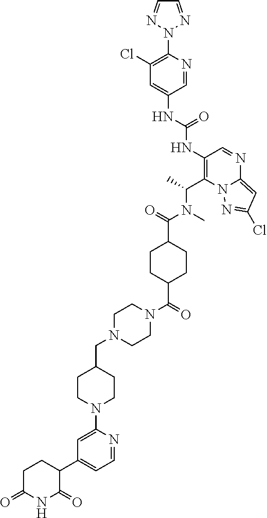 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4cc(C5CCC(=O)NC5=O)ccn4)CC3)CC2)CC1] |
| 336 | 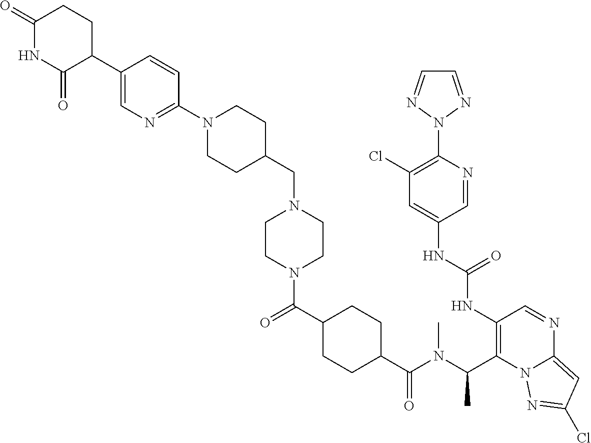 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4ccc(C5CCC(=O)NC5=O)cn4)CC3)CC2)CC1] |
| 337 | 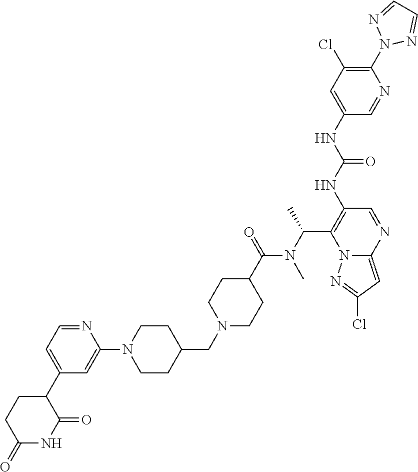 [SMILES: C[C@H](c1c(NC(=O)Nc2cnc(-n3nccn3)c(Cl)c2)cnc2cc(Cl)nn12)N(C)C(=O)C1CCN(CC2CCN(c3cc(C4CCC(=O)NC4=O)ccn3)CC2)CC1] |
| 338 | 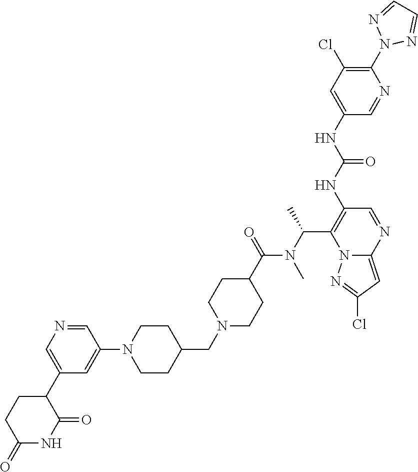 [SMILES: C[C@H](c1c(NC(=O)Nc2cnc(-n3nccn3)c(Cl)c2)cnc2cc(Cl)nn12)N(C)C(=O)C1CCN(CC2CCN(c3cncc(C4CCC(=O)NC4=O)c3)CC2)CC1] |
| 339 | 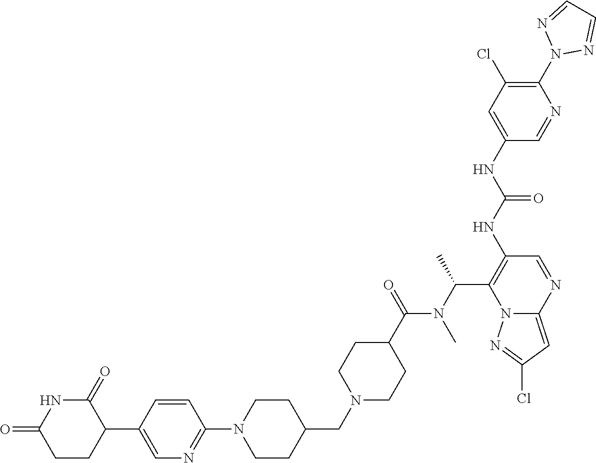 [SMILES: C[C@H](c1c(NC(=O)Nc2cnc(-n3nccn3)c(Cl)c2)cnc2cc(Cl)nn12)N(C)C(=O)C1CCN(CC2CCN(c3ccc(C4CCC(=O)NC4=O)cn3)CC2)CC1] |
| 340 | 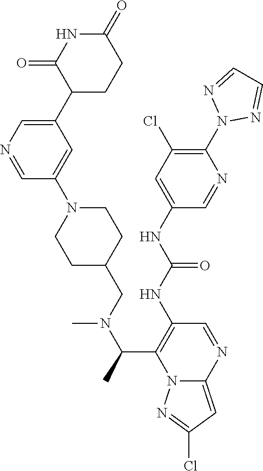 [SMILES: C[C@H](c1c(NC(=O)Nc2cnc(-n3nccn3)c(Cl)c2)cnc2cc(Cl)nn12)N(C)CC1CCN(c2cncc(C3CCC(=O)NC3=O)c2)CC1] |
| 341 | 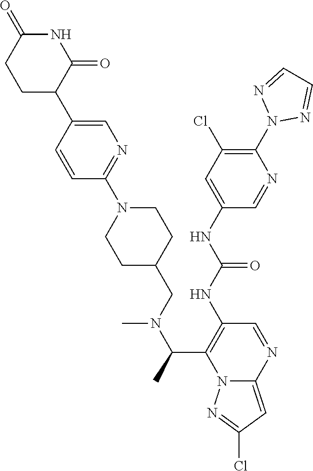 [SMILES: C[C@H](c1c(NC(=O)Nc2cnc(-n3nccn3)c(Cl)c2)cnc2cc(Cl)nn12)N(C)CC1CCN(c2ccc(C3CCC(=O)NC3=O)cn2)CC1] |
| 342 | 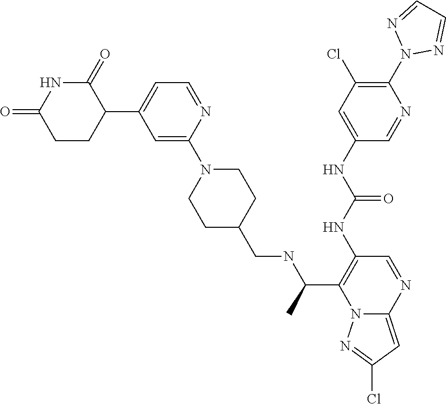 |
| 343 | 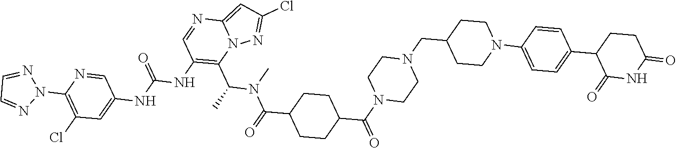 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4ccc(C5CCC(=O)NC5=O)cc4)CC3)CC2)CC1] |
| 344 | 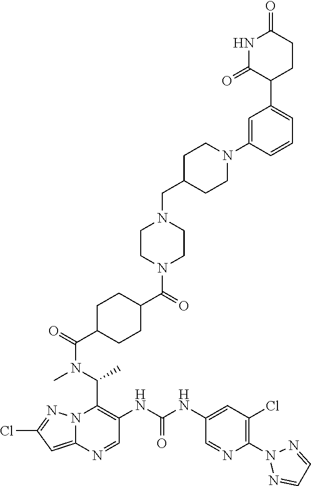 [SMILES: C[C@H](c1c(NC(=O)Nc2cnc(-n3nccn3)c(Cl)c2)cnc2cc(Cl)nn12)N(C)C(=O)C1CCC(C(=O)N2CCN(CC3CCN(c4cccc(C5CCC(=O)NC5=O)c4)CC3)CC2)CC1] |
| 345 | 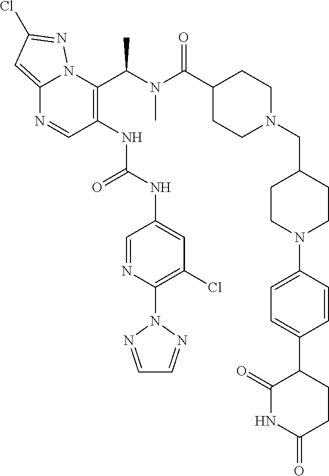 [SMILES: C[C@H](c1c(NC(=O)Nc2cnc(-n3nccn3)c(Cl)c2)cnc2cc(Cl)nn12)N(C)C(=O)C1CCN(CC2CCN(c3ccc(C4CCC(=O)NC4=O)cc3)CC2)CC1] |
| 346 | 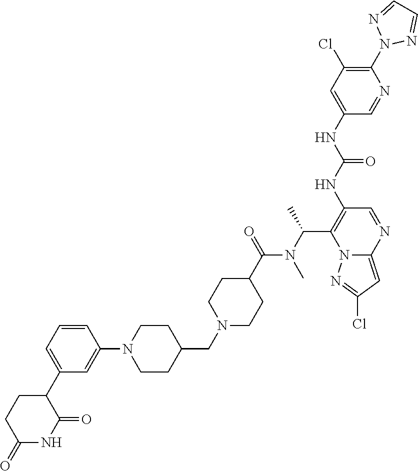 [SMILES: C[C@H](c1c(NC(=O)Nc2cnc(-n3nccn3)c(Cl)c2)cnc2cc(Cl)nn12)N(C)C(=O)C1CCN(CC2CCN(c3cccc(C4CCC(=O)NC4=O)c3)CC2)CC1] |
| 347 | 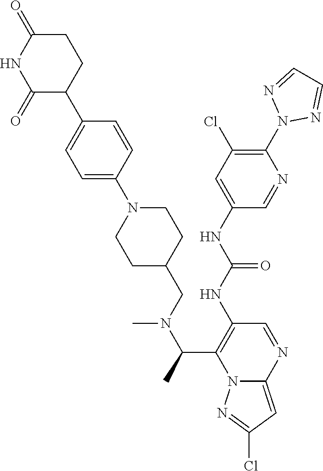 [SMILES: C[C@H](c1c(NC(=O)Nc2cnc(-n3nccn3)c(Cl)c2)cnc2cc(Cl)nn12)N(C)CC1CCN(c2ccc(C3CCC(=O)NC3=O)cc2)CC1] |
| 348 | 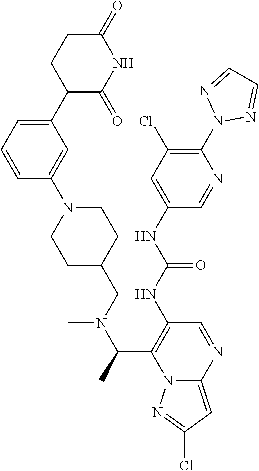 [SMILES: C[C@H](c1c(NC(=O)Nc2cnc(-n3nccn3)c(Cl)c2)cnc2cc(Cl)nn12)N(C)CC1CCN(c2cccc(C3CCC(=O)NC3=O)c2)CC1] |
| 349 | 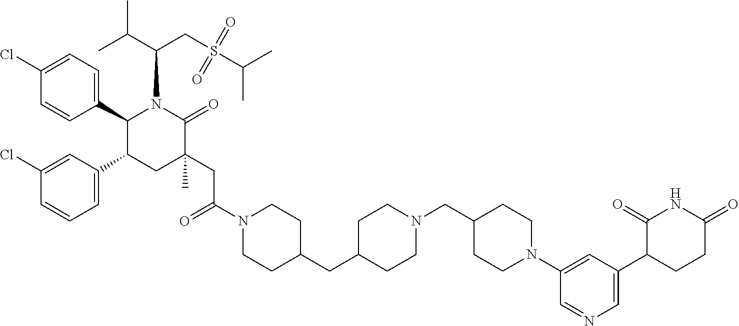 [SMILES: CC(C)[C@@H](CS(=O)(=O)C(C)C)N1C(=O)[C@@](C)(CC(=O)N2CCC(CC3CCN(CC4CCN(c5cncc(C6CCC(=O)NC6=O)c5)CC4)CC3)CC2)C[C@H](c2cccc(Cl)c2)[C@H]1c1ccc(Cl)cc1] |
| 350 | 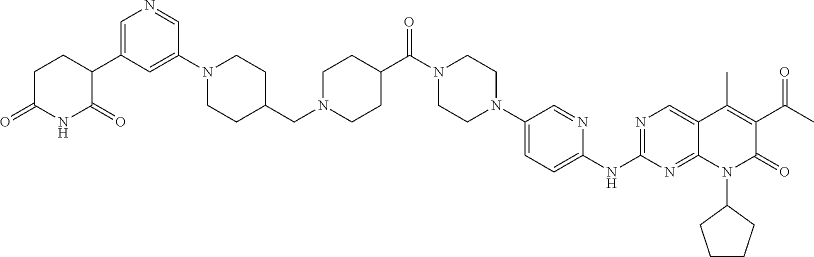 [SMILES: CC(=O)c1c(C)c2cnc(Nc3ccc(N4CCN(C(=O)C5CCN(CC6CCN(c7cncc(C8CCC(=O)NC8=O)c7)CC6)CC5)CC4)cn3)nc2n(C2CCCC2)c1=O] |
| 351 | 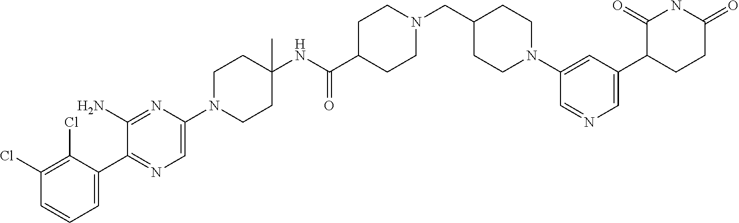 |
| 352 | 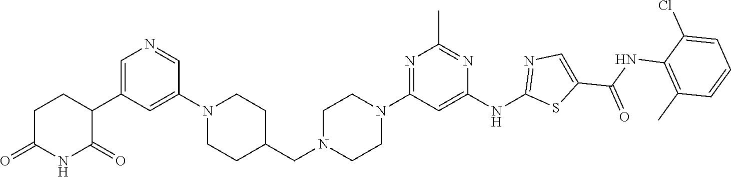 [SMILES: Cc1nc(Nc2ncc(C(=O)Nc3c(C)cccc3Cl)s2)cc(N2CCN(CC3CCN(c4cncc(C5CCC(=O)NC5=O)c4)CC3)CC2)n1] |
| 353 | 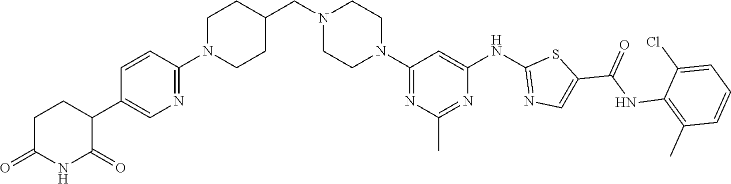 [SMILES: Cc1nc(Nc2ncc(C(=O)Nc3c(C)cccc3Cl)s2)cc(N2CCN(CC3CCN(c4ccc(C5CCC(=O)NC5=O)cn4)CC3)CC2)n1] |
| 354 | 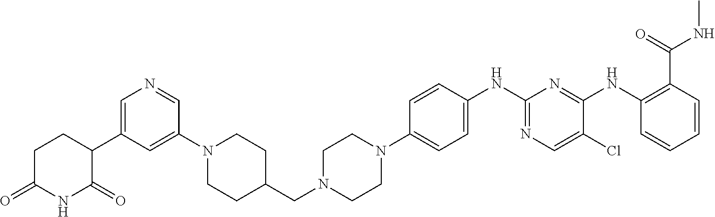 [SMILES: CNC(=O)c1ccccc1Nc1nc(Nc2ccc(N3CCN(CC4CCN(c5cncc(C6CCC(=O)NC6=O)c5)CC4)CC3)cc2)ncc1Cl] |
| 355 | 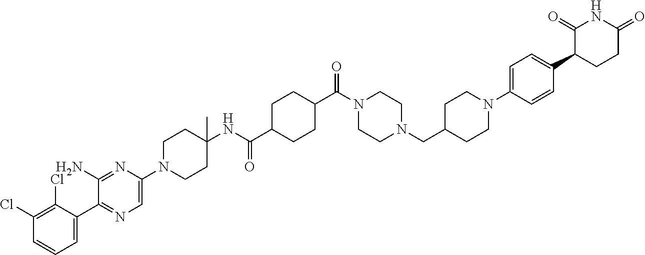 [SMILES: CC1(NC(=O)C2CCC(C(=O)N3CCN(CC4CCN(c5ccc([C@@H]6CCC(=O)NC6=O)cc5)CC4)CC3)CC2)CCN(c2cnc(-c3cccc(Cl)c3Cl)c(N)n2)CC1] |
| 356 | 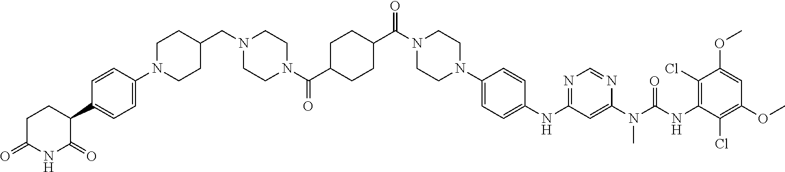 [SMILES: COc1cc(OC)c(Cl)c(NC(=O)N(C)c2cc(Nc3ccc(N4CCN(C(=O)C5CCC(C(=O)N6CCN(CC7CCN(c8ccc([C@@H]9CCC(=O)NC9=O)cc8)CC7)CC6)CC5)CC4)cc3)ncn2)c1Cl] |
| 357 | 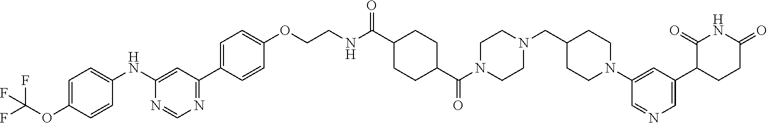 [SMILES: O=C1CCC(c2cncc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)c2)C(=O)N1] |
| 358 | 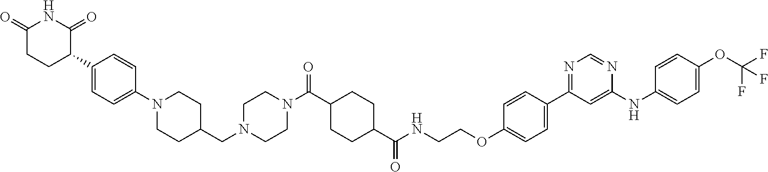 [SMILES: O=C1CC[C@@H](c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)cc2)C(=O)N1] |
| 359 | 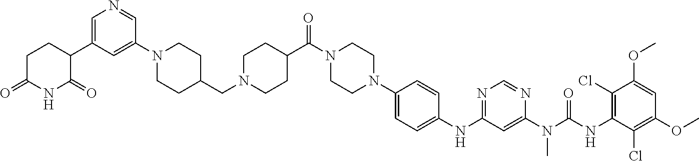 [SMILES: COc1cc(OC)c(Cl)c(NC(=O)N(C)c2cc(Nc3ccc(N4CCN(C(=O)C5CCN(CC6CCN(c7cncc(C8CCC(=O)NC8=O)c7)CC6)CC5)CC4)cc3)ncn2)c1Cl] |
| 360 | 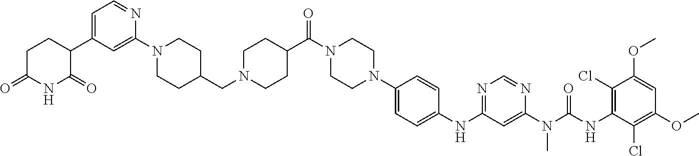 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] |
| 361 | 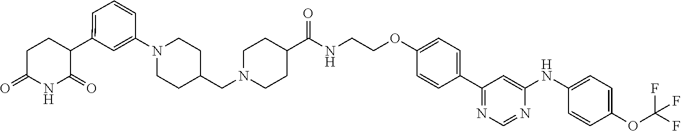 [SMILES: O=C1CCC(c2cccc(N3CCC(CN4CCC(C(=O)NCCOc5ccc(-c6cc(Nc7ccc(OC(F)(F)F)cc7)ncn6)cc5)CC4)CC3)c2)C(=O)N1] |
| 362 | 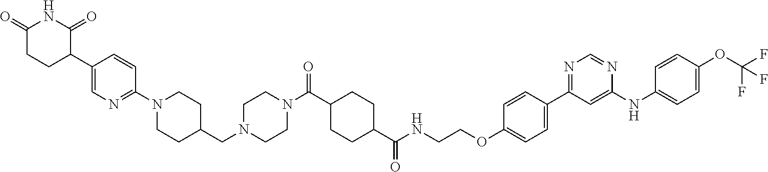 [SMILES: O=C1CCC(c2ccc(N3CCC(CN4CCN(C(=O)C5CCC(C(=O)NCCOc6ccc(-c7cc(Nc8ccc(OC(F)(F)F)cc8)ncn7)cc6)CC5)CC4)CC3)nc2)C(=O)N1] |
| 363 | 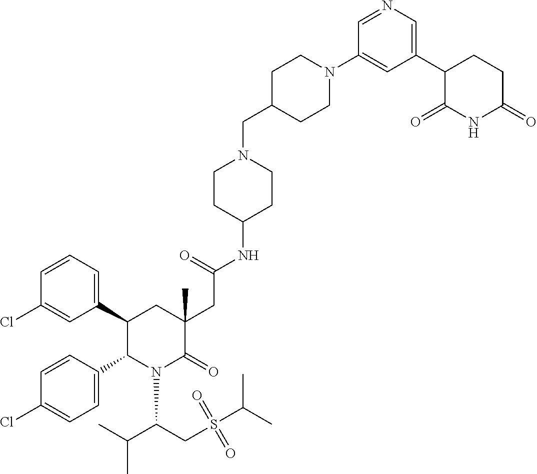 [SMILES: CC(C)[C@@H](CS(=O)(=O)C(C)C)N1C(=O)[C@@](C)(CC(=O)NC2CCN(CC3CCN(c4cncc(C5CCC(=O)NC5=O)c4)CC3)CC2)C[C@H](c2cccc(Cl)c2)[C@H]1c1ccc(Cl)cc1] |
| 364 | 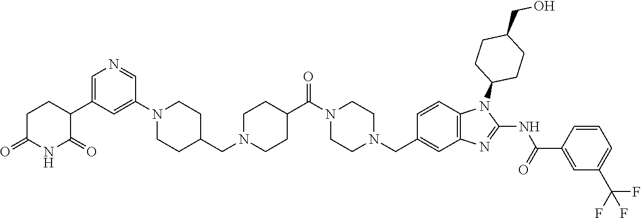 [SMILES: O=C1CCC(c2cncc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)c2)C(=O)N1] |
| 365 | 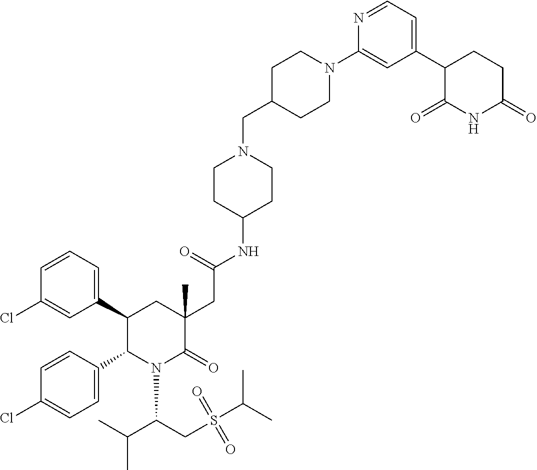 [SMILES: CC(C)[C@@H](CS(=O)(=O)C(C)C)N1C(=O)[C@@](C)(CC(=O)NC2CCN(CC3CCN(c4cc(C5CCC(=O)NC5=O)ccn4)CC3)CC2)C[C@H](c2cccc(Cl)c2)[C@H]1c1ccc(Cl)cc1] |
| 366 | 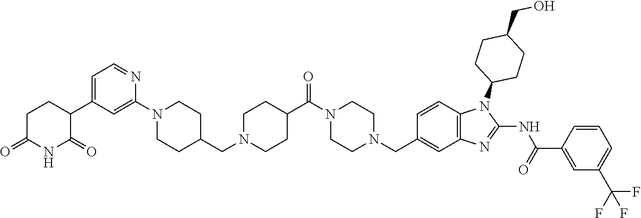 [SMILES: O=C1CCC(c2ccnc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)c2)C(=O)N1] |
| 367 | 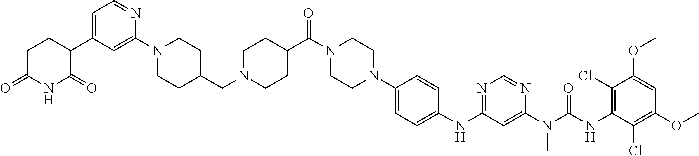 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] |
| 368 | 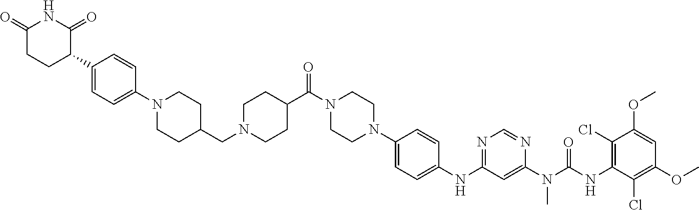 [SMILES: COc1cc(OC)c(Cl)c(NC(=O)N(C)c2cc(Nc3ccc(N4CCN(C(=O)C5CCN(CC6CCN(c7ccc([C@@H]8CCC(=O)NC8=O)cc7)CC6)CC5)CC4)cc3)ncn2)c1Cl] |
| 369 | 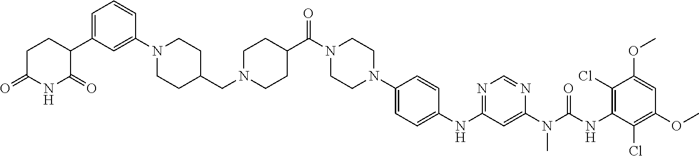 [SMILES: COc1cc(OC)c(Cl)c(NC(=O)N(C)c2cc(Nc3ccc(N4CCN(C(=O)C5CCN(CC6CCN(c7cccc(C8CCC(=O)NC8=O)c7)CC6)CC5)CC4)cc3)ncn2)c1Cl] |
| 370 | 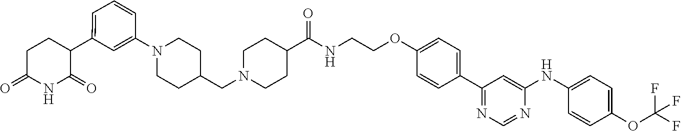 [SMILES: O=C1CCC(c2cccc(N3CCC(CN4CCC(C(=O)NCCOc5ccc(-c6cc(Nc7ccc(OC(F)(F)F)cc7)ncn6)cc5)CC4)CC3)c2)C(=O)N1] |
| 371 | 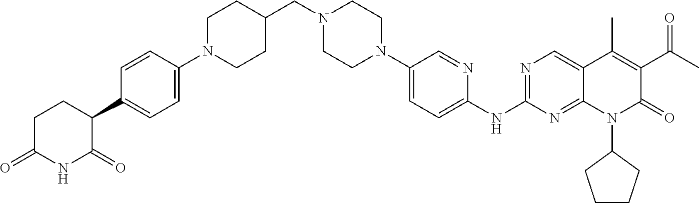 [SMILES: CC(=O)c1c(C)c2cnc(Nc3ccc(N4CCN(CC5CCN(c6ccc([C@@H]7CCC(=O)NC7=O)cc6)CC5)CC4)cn3)nc2n(C2CCCC2)c1=O] |
| 372 | 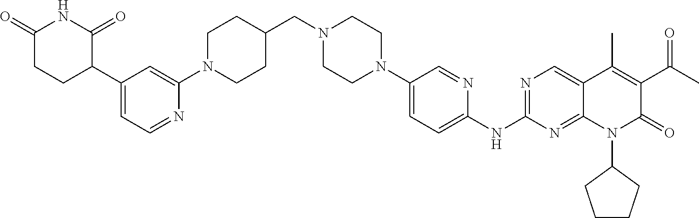 [SMILES: CC(=O)c1c(C)c2cnc(Nc3ccc(N4CCN(CC5CCN(c6cc(C7CCC(=O)NC7=O)ccn6)CC5)CC4)cn3)nc2n(C2CCCC2)c1=O] |
| 373 | 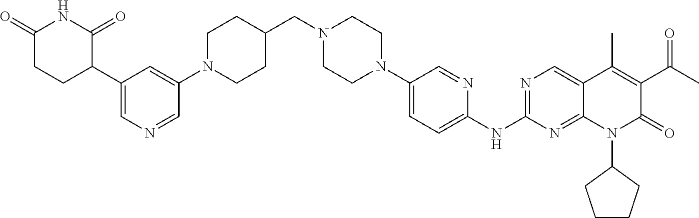 [SMILES: CC(=O)c1c(C)c2cnc(Nc3ccc(N4CCN(CC5CCN(c6cncc(C7CCC(=O)NC7=O)c6)CC5)CC4)cn3)nc2n(C2CCCC2)c1=O] |
| 374 | 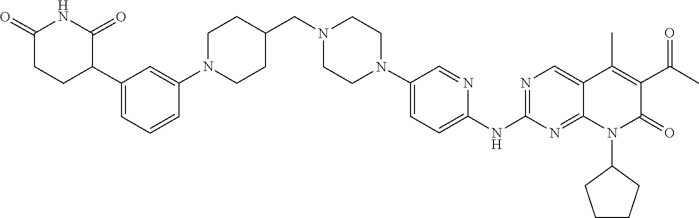 [SMILES: CC(=O)c1c(C)c2cnc(Nc3ccc(N4CCN(CC5CCN(c6cccc(C7CCC(=O)NC7=O)c6)CC5)CC4)cn3)nc2n(C2CCCC2)c1=O] |
| 375 | 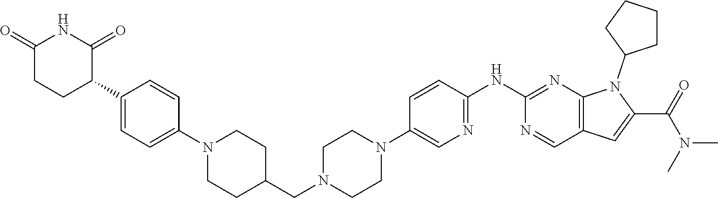 [SMILES: CN(C)C(=O)c1cc2cnc(Nc3ccc(N4CCN(CC5CCN(c6ccc([C@@H]7CCC(=O)NC7=O)cc6)CC5)CC4)cn3)nc2n1C1CCCC1] |
| 376 | 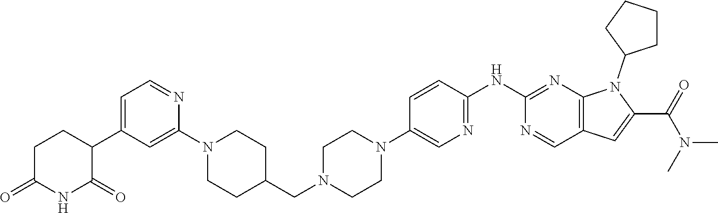 [SMILES: CN(C)C(=O)c1cc2cnc(Nc3ccc(N4CCN(CC5CCN(c6cc(C7CCC(=O)NC7=O)ccn6)CC5)CC4)cn3)nc2n1C1CCCC1] |
| 377 | 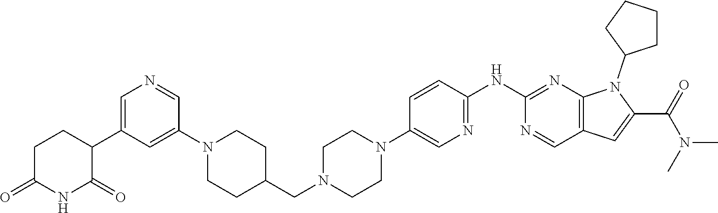 [SMILES: CN(C)C(=O)c1cc2cnc(Nc3ccc(N4CCN(CC5CCN(c6cncc(C7CCC(=O)NC7=O)c6)CC5)CC4)cn3)nc2n1C1CCCC1] |
| 378 | 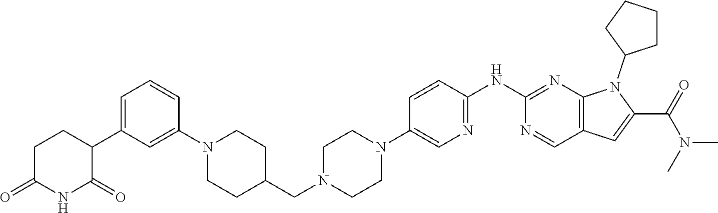 [SMILES: CN(C)C(=O)c1cc2cnc(Nc3ccc(N4CCN(CC5CCN(c6cccc(C7CCC(=O)NC7=O)c6)CC5)CC4)cn3)nc2n1C1CCCC1] |
| 379 | 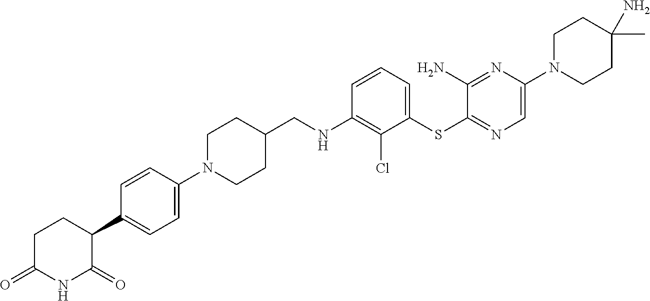 [SMILES: CC1(N)CCN(c2cnc(Sc3cccc(NCC4CCN(c5ccc([C@@H]6CCC(=O)NC6=O)cc5)CC4)c3Cl)c(N)n2)CC1] |
| 380 | 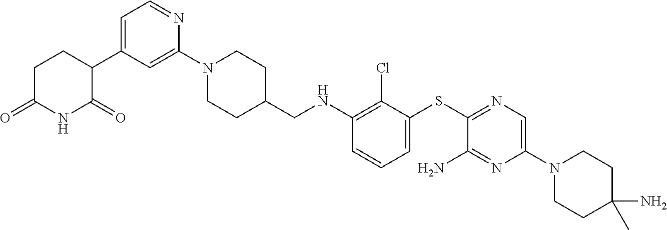 [SMILES: CC1(N)CCN(c2cnc(Sc3cccc(NCC4CCN(c5cc(C6CCC(=O)NC6=O)ccn5)CC4)c3Cl)c(N)n2)CC1] |
| 381 | 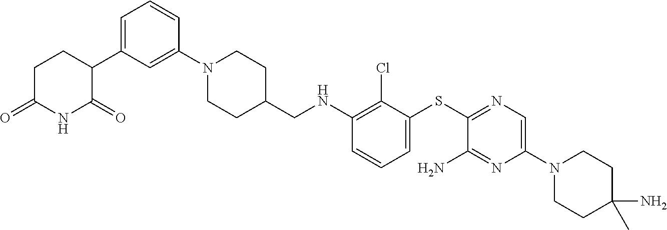 [SMILES: CC1(N)CCN(c2cnc(Sc3cccc(NCC4CCN(c5cccc(C6CCC(=O)NC6=O)c5)CC4)c3Cl)c(N)n2)CC1] |
| 382 | 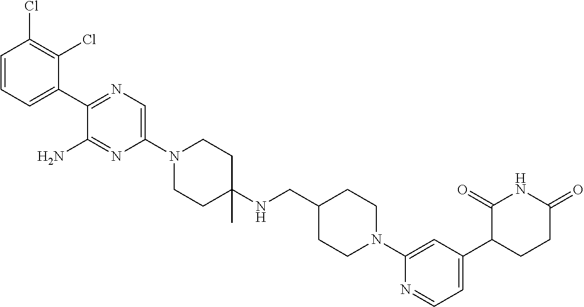 [SMILES: CC1(NCC2CCN(c3cc(C4CCC(=O)NC4=O)ccn3)CC2)CCN(c2cnc(-c3cccc(Cl)c3Cl)c(N)n2)CC1] |
| 383 | 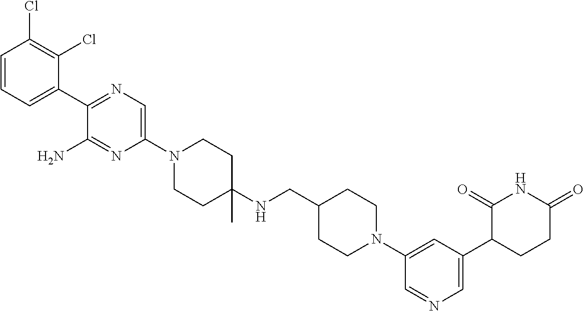 [SMILES: CC1(NCC2CCN(c3cncc(C4CCC(=O)NC4=O)c3)CC2)CCN(c2cnc(-c3cccc(Cl)c3Cl)c(N)n2)CC1] |
| 384 | 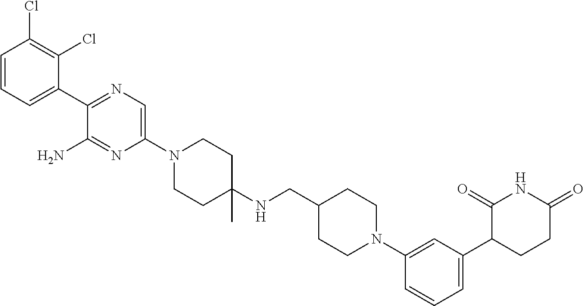 [SMILES: CC1(NCC2CCN(c3cccc(C4CCC(=O)NC4=O)c3)CC2)CCN(c2cnc(-c3cccc(Cl)c3Cl)c(N)n2)CC1] |
| 385 | 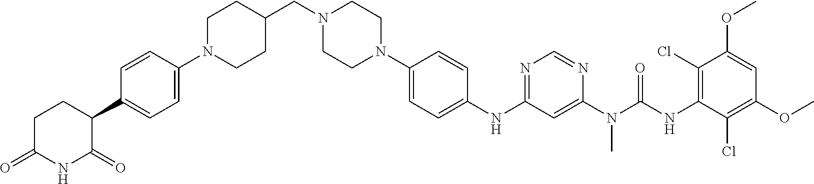 [SMILES: COc1cc(OC)c(Cl)c(NC(=O)N(C)c2cc(Nc3ccc(N4CCN(CC5CCN(c6ccc([C@@H]7CCC(=O)NC7=O)cc6)CC5)CC4)cc3)ncn2)c1Cl] |
| 386 | 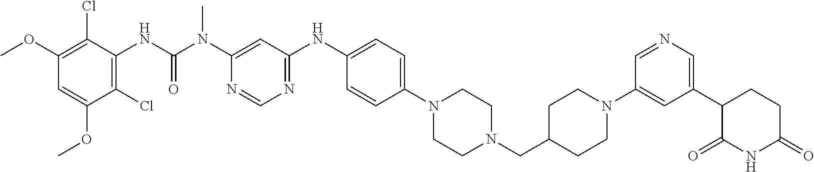 [SMILES: COc1cc(OC)c(Cl)c(NC(=O)N(C)c2cc(Nc3ccc(N4CCN(CC5CCN(c6cncc(C7CCC(=O)NC7=O)c6)CC5)CC4)cc3)ncn2)c1Cl] |
| 387 | 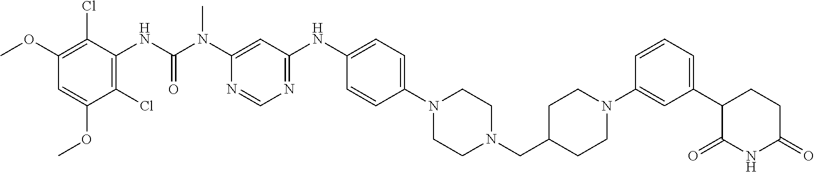 [SMILES: COc1cc(OC)c(Cl)c(NC(=O)N(C)c2cc(Nc3ccc(N4CCN(CC5CCN(c6cccc(C7CCC(=O)NC7=O)c6)CC5)CC4)cc3)ncn2)c1Cl] |
| 388 | 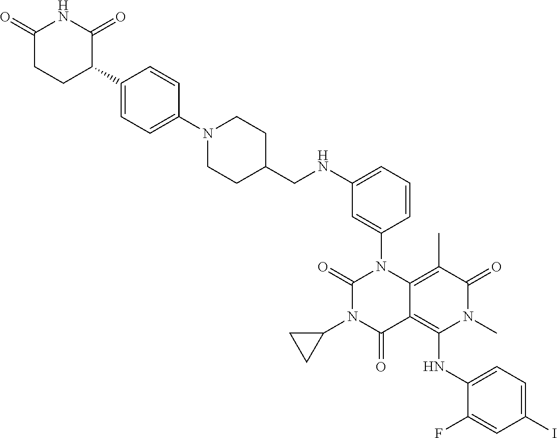 [SMILES: Cc1c(=O)n(C)c(Nc2ccc(I)cc2F)c2c(=O)n(C3CC3)c(=O)n(-c3cccc(NCC4CCN(c5ccc([C@@H]6CCC(=O)NC6=O)cc5)CC4)c3)c12] |
| 389 | 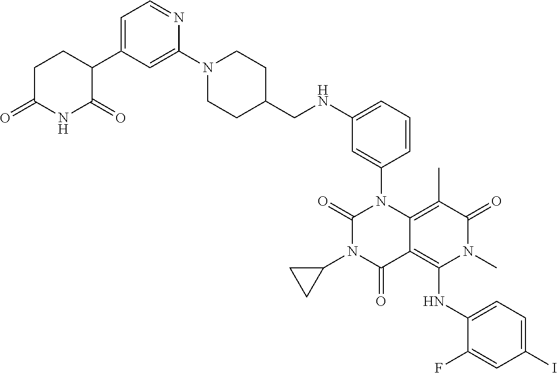 [SMILES: Cc1c(=O)n(C)c(Nc2ccc(I)cc2F)c2c(=O)n(C3CC3)c(=O)n(-c3cccc(NCC4CCN(c5cc(C6CCC(=O)NC6=O)ccn5)CC4)c3)c12] |
| 390 | 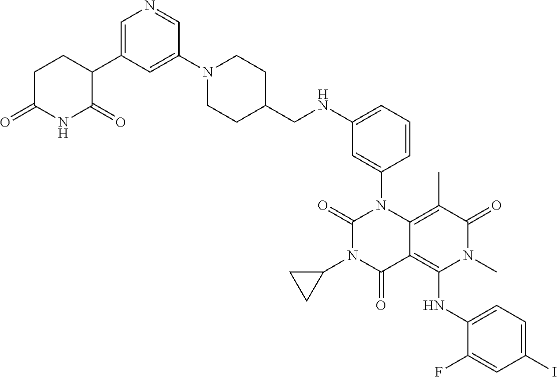 [SMILES: Cc1c(=O)n(C)c(Nc2ccc(I)cc2F)c2c(=O)n(C3CC3)c(=O)n(-c3cccc(NCC4CCN(c5cncc(C6CCC(=O)NC6=O)c5)CC4)c3)c12] |
| 391 | 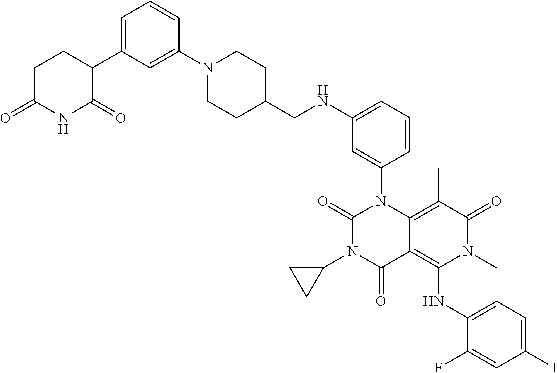 [SMILES: Cc1c(=O)n(C)c(Nc2ccc(I)cc2F)c2c(=O)n(C3CC3)c(=O)n(-c3cccc(NCC4CCN(c5cccc(C6CCC(=O)NC6=O)c5)CC4)c3)c12] |
| 392 | 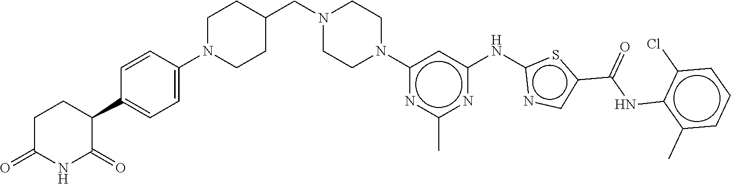 |
| 393 | 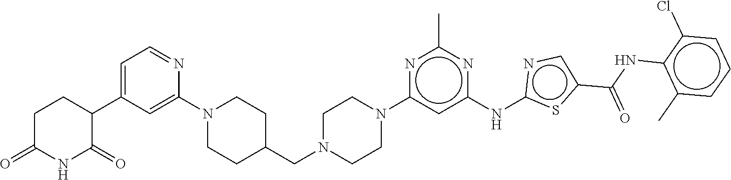 |
| 394 | 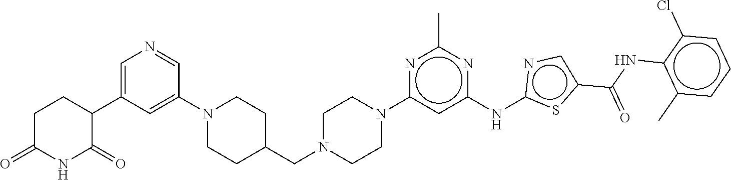 |
| 395 | 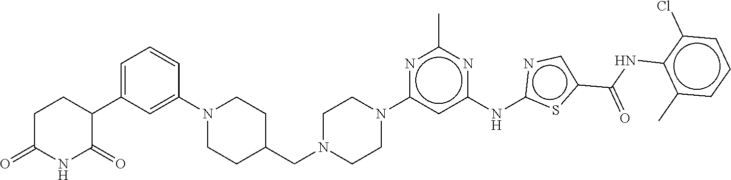 |
| 396 | 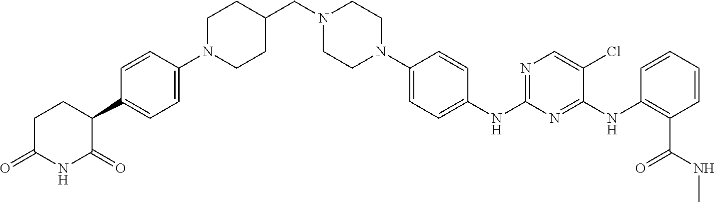 [SMILES: CNC(=O)c1ccccc1Nc1nc(Nc2ccc(N3CCN(CC4CCN(c5ccc([C@@H]6CCC(=O)NC6=O)cc5)CC4)CC3)cc2)ncc1Cl] |
| 397 | 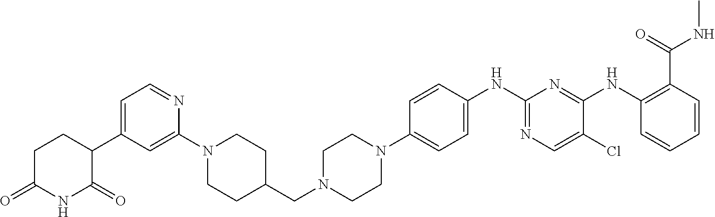 [SMILES: CNC(=O)c1ccccc1Nc1nc(Nc2ccc(N3CCN(CC4CCN(c5cc(C6CCC(=O)NC6=O)ccn5)CC4)CC3)cc2)ncc1Cl] |
| 398 | 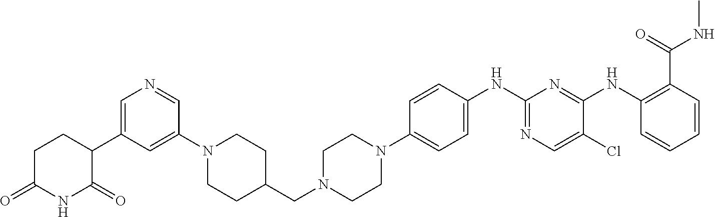 [SMILES: CNC(=O)c1ccccc1Nc1nc(Nc2ccc(N3CCN(CC4CCN(c5cncc(C6CCC(=O)NC6=O)c5)CC4)CC3)cc2)ncc1Cl] |
| 399 | 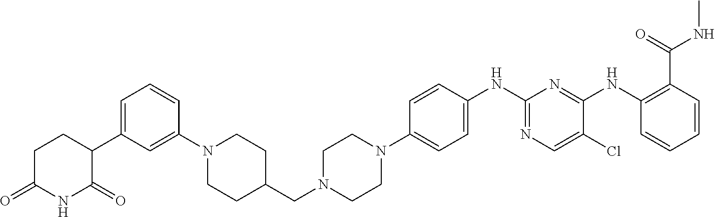 [SMILES: CNC(=O)c1ccccc1Nc1nc(Nc2ccc(N3CCN(CC4CCN(c5cccc(C6CCC(=O)NC6=O)c5)CC4)CC3)cc2)ncc1Cl] |
| 400 | 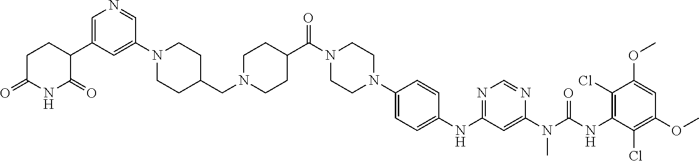 [SMILES: COc1cc(OC)c(Cl)c(NC(=O)N(C)c2cc(Nc3ccc(N4CCN(C(=O)C5CCN(CC6CCN(c7cncc(C8CCC(=O)NC8=O)c7)CC6)CC5)CC4)cc3)ncn2)c1Cl] |
| 401 | 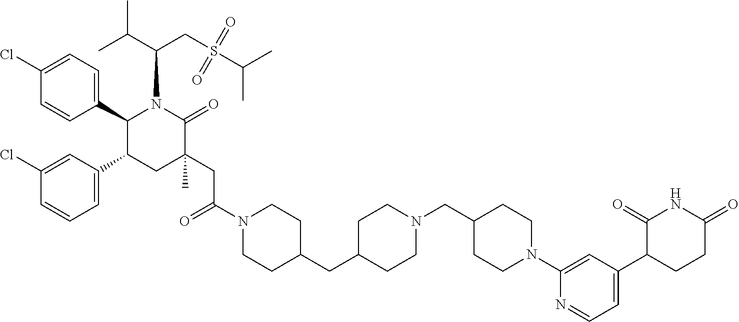 [SMILES: CC(C)[C@@H](CS(=O)(=O)C(C)C)N1C(=O)[C@@](C)(CC(=O)N2CCC(CC3CCN(CC4CCN(c5cc(C6CCC(=O)NC6=O)ccn5)CC4)CC3)CC2)C[C@H](c2cccc(Cl)c2)[C@H]1c1ccc(Cl)cc1] |
| 402 | 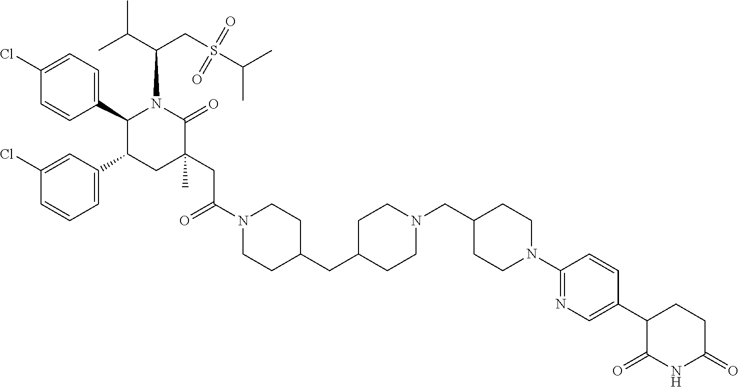 [SMILES: CC(C)[C@@H](CS(=O)(=O)C(C)C)N1C(=O)[C@@](C)(CC(=O)N2CCC(CC3CCN(CC4CCN(c5ccc(C6CCC(=O)NC6=O)cn5)CC4)CC3)CC2)C[C@H](c2cccc(Cl)c2)[C@H]1c1ccc(Cl)cc1] |
| 403 | 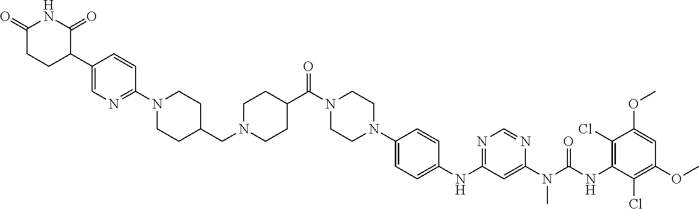 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cn7)CC6)CC5)CC4)cc3)ncn2)c1Cl] |
| 404 | 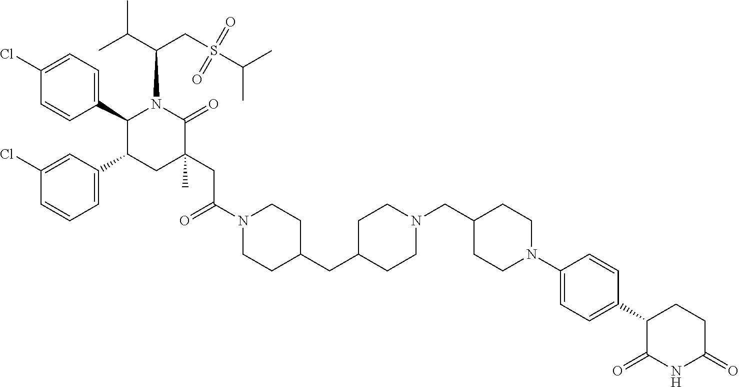 [SMILES: CC(C)[C@@H](CS(=O)(=O)C(C)C)N1C(=O)[C@@](C)(CC(=O)N2CCC(CC3CCN(CC4CCN(c5ccc([C@@H]6CCC(=O)NC6=O)cc5)CC4)CC3)CC2)C[C@H](c2cccc(Cl)c2)[C@H]1c1ccc(Cl)cc1] |
| 405 | 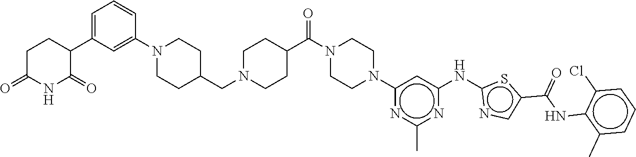 |
| 406 | 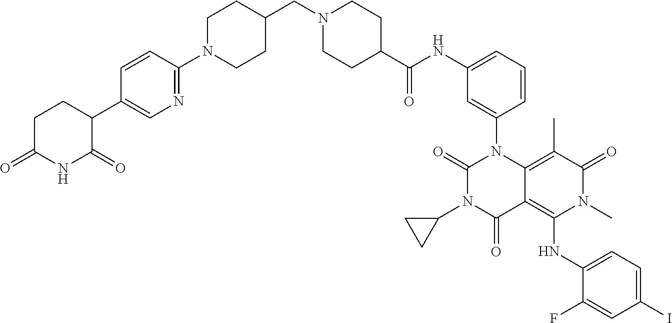 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] |
| 407 | 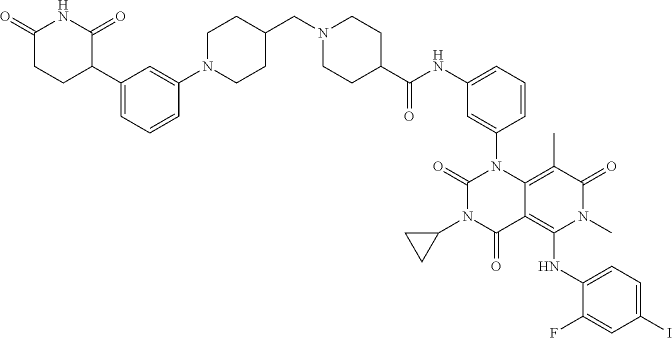 [SMILES: Cc1c(=O)n(C)c(Nc2ccc(I)cc2F)c2c(=O)n(C3CC3)c(=O)n(-c3cccc(NC(=O)C4CCN(CC5CCN(c6cccc(C7CCC(=O)NC7=O)c6)CC5)CC4)c3)c12] |
| 408 | 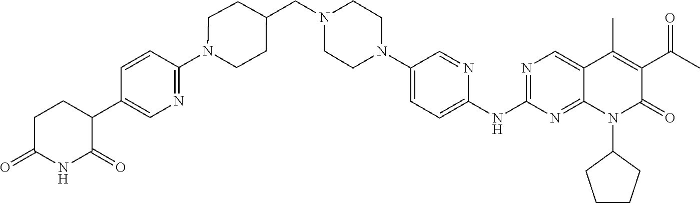 [SMILES: CC(=O)c1c(C)c2cnc(Nc3ccc(N4CCN(CC5CCN(c6ccc(C7CCC(=O)NC7=O)cn6)CC5)CC4)cn3)nc2n(C2CCCC2)c1=O] |
| 409 | 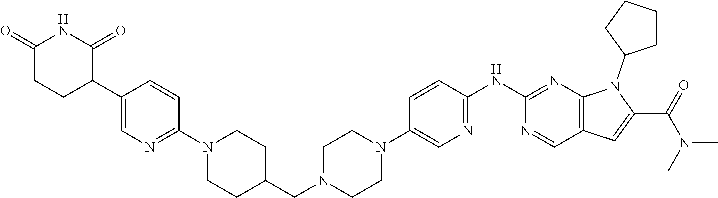 [SMILES: CN(C)C(=O)c1cc2cnc(Nc3ccc(N4CCN(CC5CCN(c6ccc(C7CCC(=O)NC7=O)cn6)CC5)CC4)cn3)nc2n1C1CCCC1] |
| 410 | 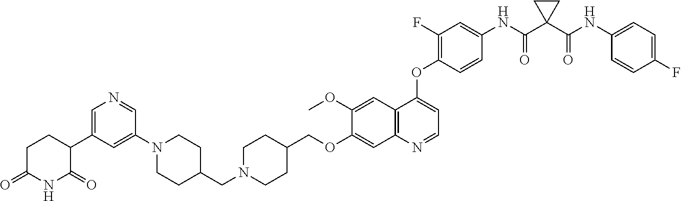 [SMILES: COc1cc2c(Oc3ccc(NC(=O)C4(C(=O)Nc5ccc(F)cc5)CC4)cc3F)ccnc2cc1OCC1CCN(CC2CCN(c3cncc(C4CCC(=O)NC4=O)c3)CC2)CC1] |
| 411 | 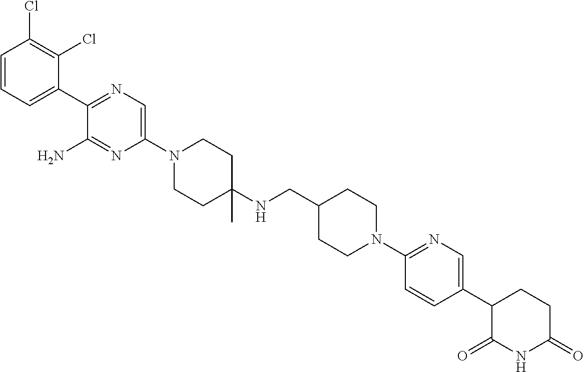 [SMILES: CC1(NCC2CCN(c3ccc(C4CCC(=O)NC4=O)cn3)CC2)CCN(c2cnc(-c3cccc(Cl)c3Cl)c(N)n2)CC1] |
| 412 | 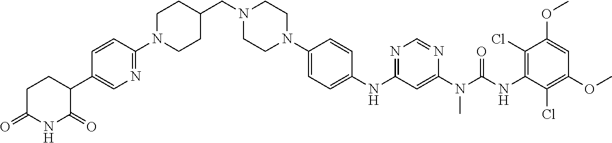 [SMILES: COc1cc(OC)c(Cl)c(NC(=O)N(C)c2cc(Nc3ccc(N4CCN(CC5CCN(c6ccc(C7CCC(=O)NC7=O)cn6)CC5)CC4)cc3)ncn2)c1Cl] |
| 413 | 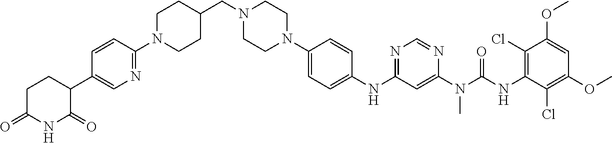 [SMILES: COc1cc(OC)c(Cl)c(NC(=O)N(C)c2cc(Nc3ccc(N4CCN(CC5CCN(c6ccc(C7CCC(=O)NC7=O)cn6)CC5)CC4)cc3)ncn2)c1Cl] |
| 414 | 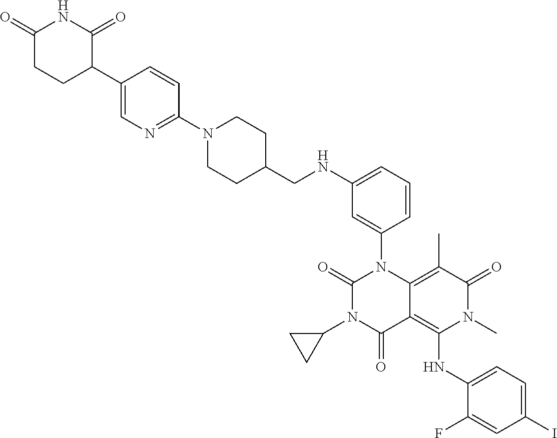 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cn5)CC4)c3)c12] |
| 415 | 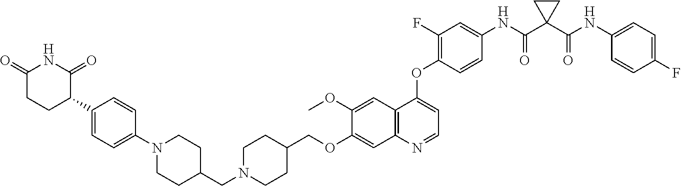 [SMILES: COc1cc2c(Oc3ccc(NC(=O)C4(C(=O)Nc5ccc(F)cc5)CC4)cc3F)ccnc2cc1OCC1CCN(CC2CCN(c3ccc([C@@H]4CCC(=O)NC4=O)cc3)CC2)CC1] |
| 416 | 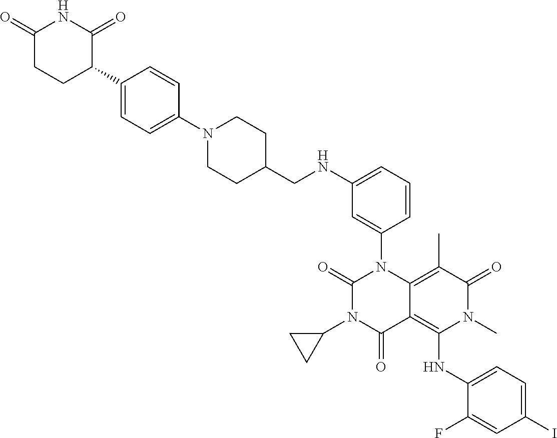 [SMILES: Cc1c(=O)n(C)c(Nc2ccc(I)cc2F)c2c(=O)n(C3CC3)c(=O)n(-c3cccc(NCC4CCN(c5ccc([C@@H]6CCC(=O)NC6=O)cc5)CC4)c3)c12] |
| 417 | 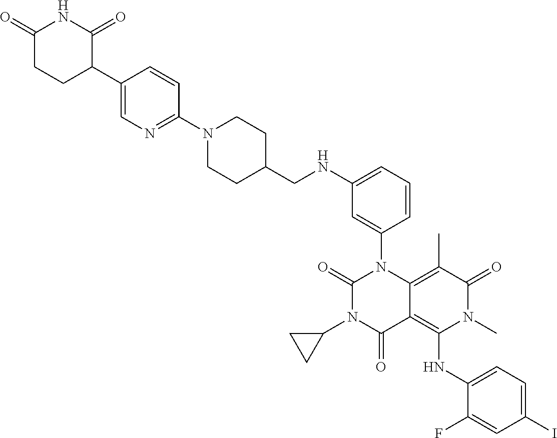 [SMILES: Cc1c(=O)n(C)c(Nc2ccc(I)cc2F)c2c(=O)n(C3CC3)c(=O)n(-c3cccc(NCC4CCN(c5ccc(C6CCC(=O)NC6=O)cn5)CC4)c3)c12] |
| 418 | 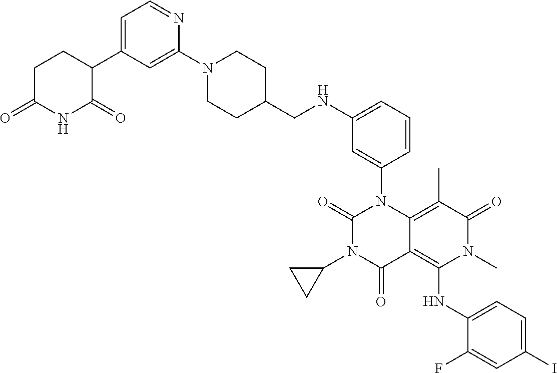 [SMILES: Cc1c(=O)n(C)c(Nc2ccc(I)cc2F)c2c(=O)n(C3CC3)c(=O)n(-c3cccc(NCC4CCN(c5cc(C6CCC(=O)NC6=O)ccn5)CC4)c3)c12] |
| 419 | 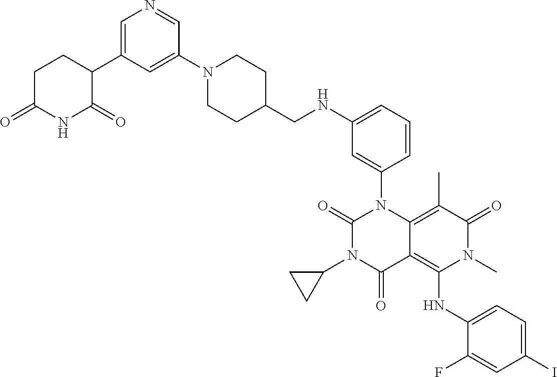 [SMILES: Cc1c(=O)n(C)c(Nc2ccc(I)cc2F)c2c(=O)n(C3CC3)c(=O)n(-c3cccc(NCC4CCN(c5cncc(C6CCC(=O)NC6=O)c5)CC4)c3)c12] |
| 420 | 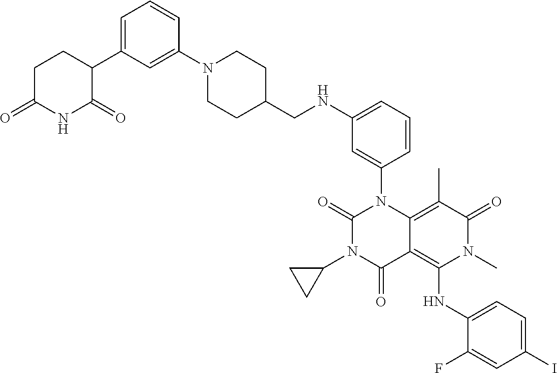 [SMILES: Cc1c(=O)n(C)c(Nc2ccc(I)cc2F)c2c(=O)n(C3CC3)c(=O)n(-c3cccc(NCC4CCN(c5cccc(C6CCC(=O)NC6=O)c5)CC4)c3)c12] |
| 421 | 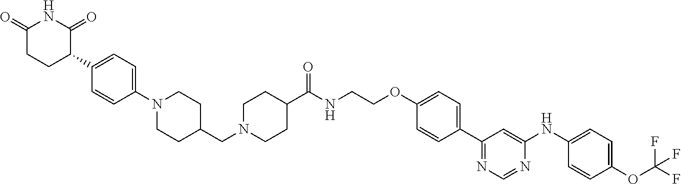 [SMILES: O=C1CC[C@@H](c2ccc(N3CCC(CN4CCC(C(=O)NCCOc5ccc(-c6cc(Nc7ccc(OC(F)(F)F)cc7)ncn6)cc5)CC4)CC3)cc2)C(=O)N1] |
| 422 | 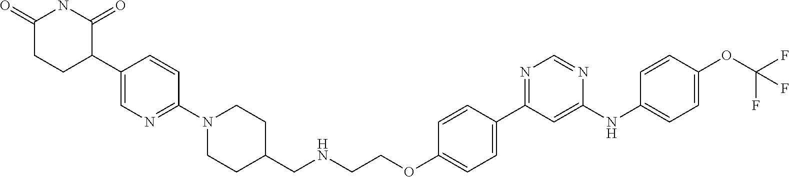 |
| 423 | 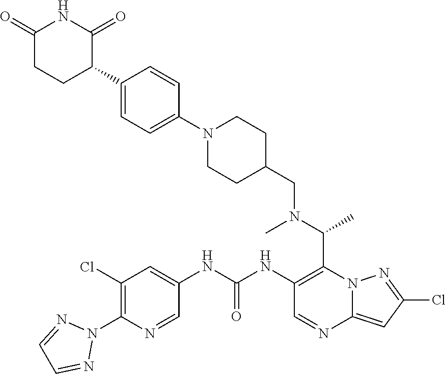 [SMILES: C[C@H](c1c(NC(=O)Nc2cnc(-n3nccn3)c(Cl)c2)cnc2cc(Cl)nn12)N(C)CC1CCN(c2ccc([C@@H]3CCC(=O)NC3=O)cc2)CC1] |
| 424 | 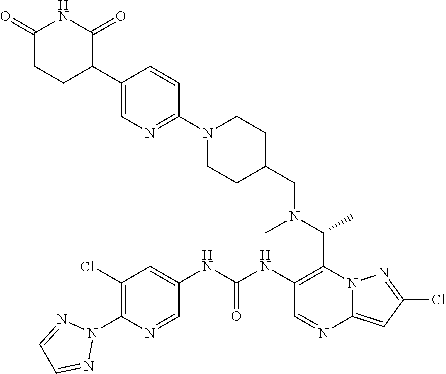 [SMILES: C[C@H](c1c(NC(=O)Nc2cnc(-n3nccn3)c(Cl)c2)cnc2cc(Cl)nn12)N(C)CC1CCN(c2ccc(C3CCC(=O)NC3=O)cn2)CC1] |
| 425 | 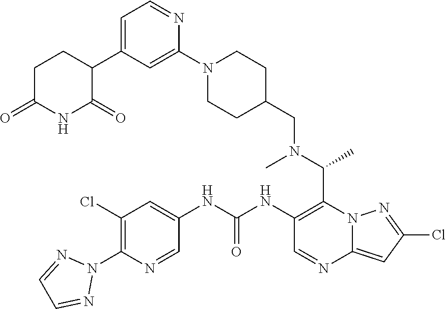 [SMILES: C[C@H](c1c(NC(=O)Nc2cnc(-n3nccn3)c(Cl)c2)cnc2cc(Cl)nn12)N(C)CC1CCN(c2cc(C3CCC(=O)NC3=O)ccn2)CC1] |
| 426 | 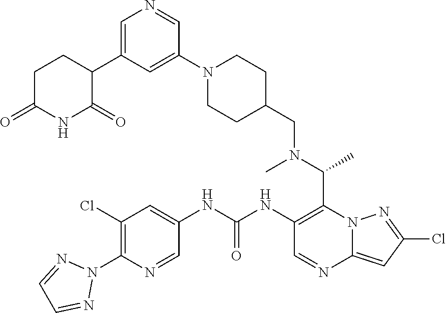 [SMILES: C[C@H](c1c(NC(=O)Nc2cnc(-n3nccn3)c(Cl)c2)cnc2cc(Cl)nn12)N(C)CC1CCN(c2cncc(C3CCC(=O)NC3=O)c2)CC1] |
| 427 | 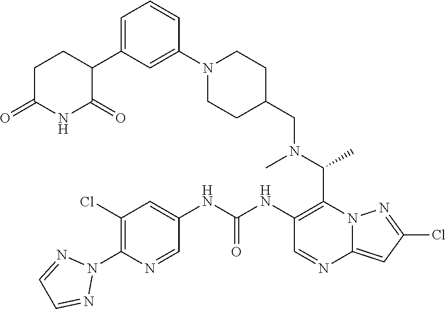 [SMILES: C[C@H](c1c(NC(=O)Nc2cnc(-n3nccn3)c(Cl)c2)cnc2cc(Cl)nn12)N(C)CC1CCN(c2cccc(C3CCC(=O)NC3=O)c2)CC1] |
| 428 | 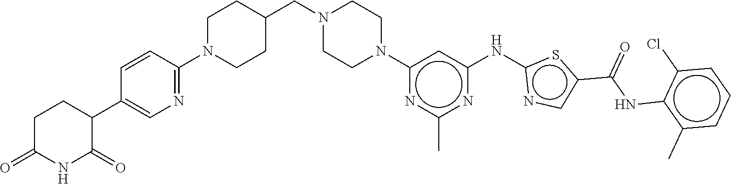 |
| 429 | 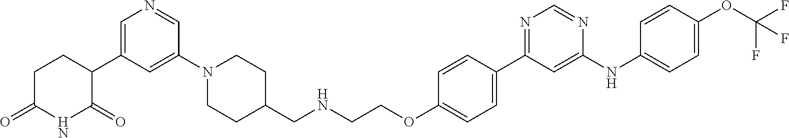 [SMILES: O=C1CCC(c2cncc(N3CCC(CNCCOc4ccc(-c5cc(Nc6ccc(OC(F)(F)F)cc6)ncn5)cc4)CC3)c2)C(=O)N1] |
| 430 | 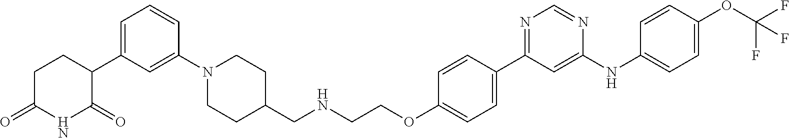 [SMILES: O=C1CCC(c2cccc(N3CCC(CNCCOc4ccc(-c5cc(Nc6ccc(OC(F)(F)F)cc6)ncn5)cc4)CC3)c2)C(=O)N1] |
| 431 | 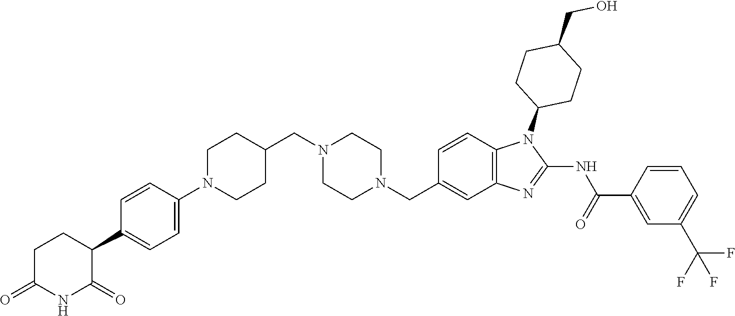 [SMILES: O=C1CC[C@@H](c2ccc(N3CCC(CN4CCN(Cc5ccc6c(c5)nc(NC(=O)c5cccc(C(F)(F)F)c5)n6[C@H]5CC[C@@H](CO)CC5)CC4)CC3)cc2)C(=O)N1] |
| 432 | 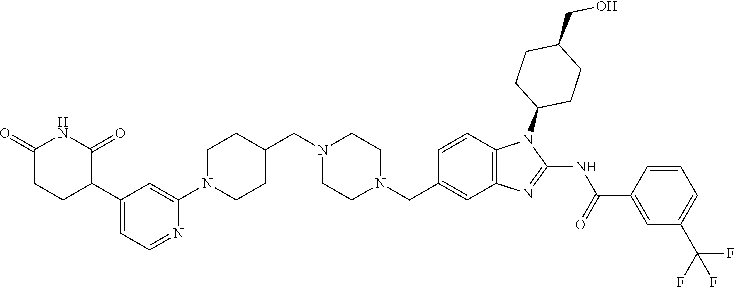 [SMILES: O=C1CCC(c2ccnc(N3CCC(CN4CCN(Cc5ccc6c(c5)nc(NC(=O)c5cccc(C(F)(F)F)c5)n6[C@H]5CC[C@@H](CO)CC5)CC4)CC3)c2)C(=O)N1] |
| 433 | 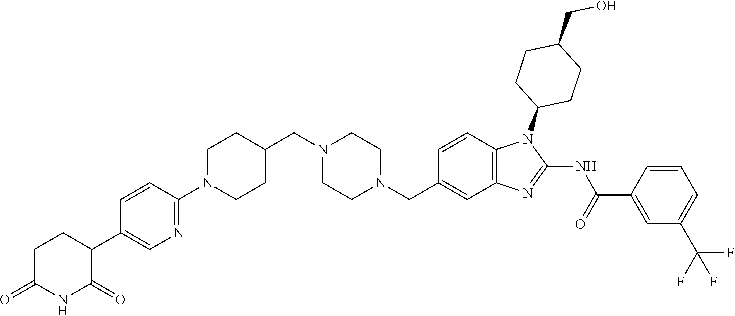 [SMILES: O=C1CCC(c2ccc(N3CCC(CN4CCN(Cc5ccc6c(c5)nc(NC(=O)c5cccc(C(F)(F)F)c5)n6[C@H]5CC[C@@H](CO)CC5)CC4)CC3)nc2)C(=O)N1] |
| 434 | 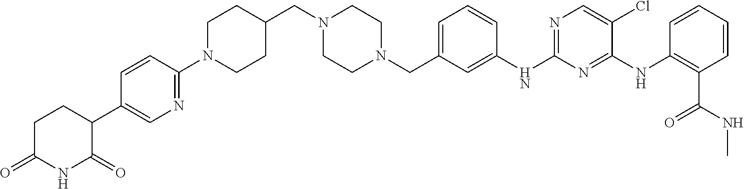 [SMILES: CNC(=O)c1ccccc1Nc1nc(Nc2cccc(CN3CCN(CC4CCN(c5ccc(C6CCC(=O)NC6=O)cn5)CC4)CC3)c2)ncc1Cl] |
| 435 | 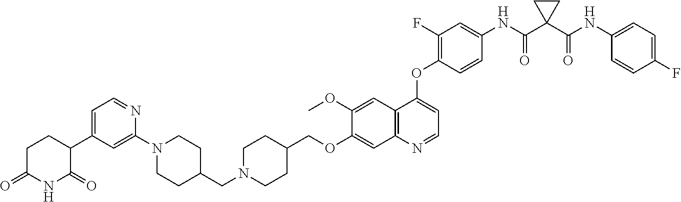 [SMILES: COc1cc2c(Oc3ccc(NC(=O)C4(C(=O)Nc5ccc(F)cc5)CC4)cc3F)ccnc2cc1OCC1CCN(CC2CCN(c3cc(C4CCC(=O)NC4=O)ccn3)CC2)CC1] |
| 436 | 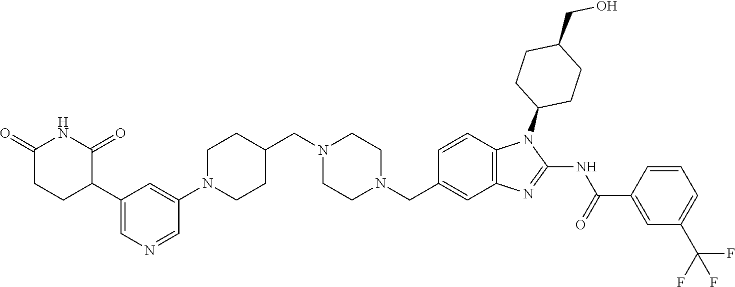 [SMILES: O=C1CCC(c2cncc(N3CCC(CN4CCN(Cc5ccc6c(c5)nc(NC(=O)c5cccc(C(F)(F)F)c5)n6[C@H]5CC[C@@H](CO)CC5)CC4)CC3)c2)C(=O)N1] |
| 437 | 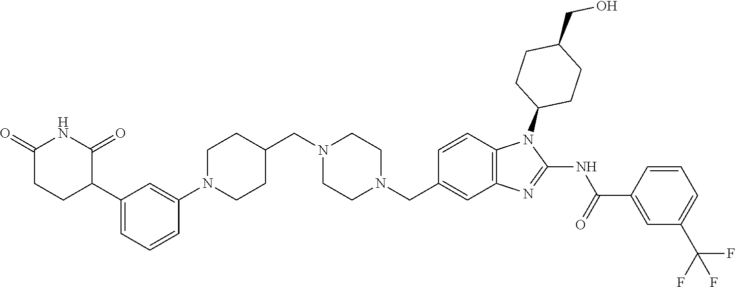 [SMILES: O=C1CCC(c2cccc(N3CCC(CN4CCN(Cc5ccc6c(c5)nc(NC(=O)c5cccc(C(F)(F)F)c5)n6[C@H]5CC[C@@H](CO)CC5)CC4)CC3)c2)C(=O)N1] |
| 438 | 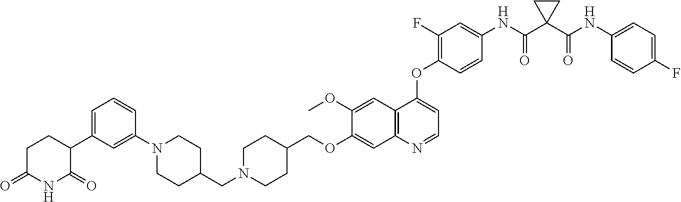 [SMILES: COc1cc2c(Oc3ccc(NC(=O)C4(C(=O)Nc5ccc(F)cc5)CC4)cc3F)ccnc2cc1OCC1CCN(CC2CCN(c3cccc(C4CCC(=O)NC4=O)c3)CC2)CC1] |
| 439 | 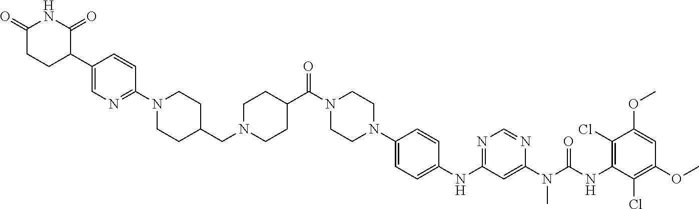 [SMILES: COc1cc(OC)c(Cl)c(NC(=O)N(C)c2cc(Nc3ccc(N4CCN(C(=O)C5CCN(CC6CCN(c7ccc(C8CCC(=O)NC8=O)cn7)CC6)CC5)CC4)cc3)ncn2)c1Cl] |
| 440 | 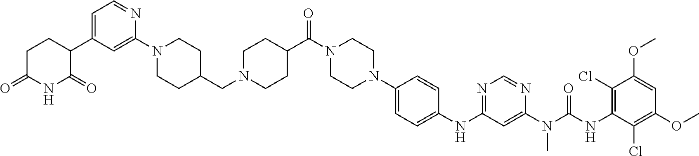 [SMILES: COc1cc(OC)c(Cl)c(NC(=O)N(C)c2cc(Nc3ccc(N4CCN(C(=O)C5CCN(CC6CCN(c7cc(C8CCC(=O)NC8=O)ccn7)CC6)CC5)CC4)cc3)ncn2)c1Cl] |
| 441 | 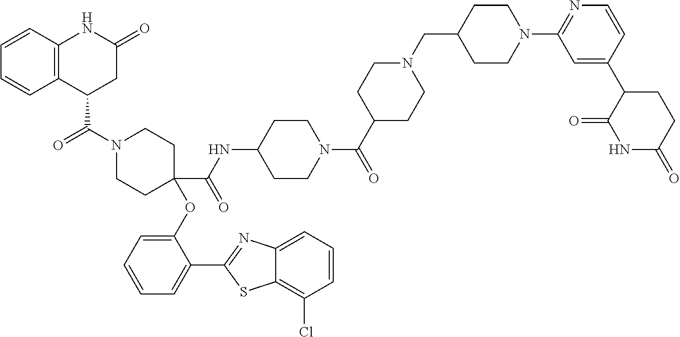 [SMILES: O=C1CCC(c2ccnc(N3CCC(CN4CCC(C(=O)N5CCC(NC(=O)C6(Oc7ccccc7-c7nc8cccc(Cl)c8s7)CCN(C(=O)[C@H]7CC(=O)Nc8ccccc87)CC6)CC5)CC4)CC3)c2)C(=O)N1] |
| 442 | 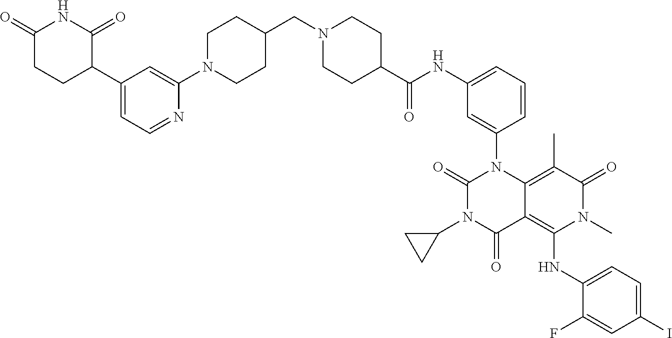 [SMILES: Cc1c(=O)n(C)c(Nc2ccc(I)cc2F)c2c(=O)n(C3CC3)c(=O)n(-c3cccc(NC(=O)C4CCN(CC5CCN(c6cc(C7CCC(=O)NC7=O)ccn6)CC5)CC4)c3)c12] |
| 443 | 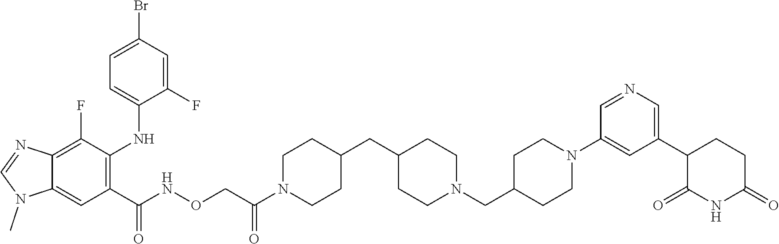 [SMILES: Cn1cnc2c(F)c(Nc3ccc(Br)cc3F)c(C(=O)NOCC(=O)N3CCC(CC4CCN(CC5CCN(c6cncc(C7CCC(=O)NC7=O)c6)CC5)CC4)CC3)cc21] |
| 444 | 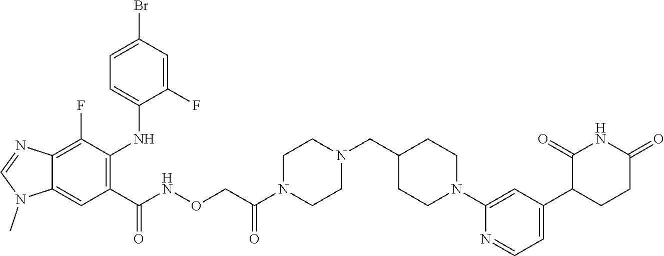 [SMILES: Cn1cnc2c(F)c(Nc3ccc(Br)cc3F)c(C(=O)NOCC(=O)N3CCN(CC4CCN(c5cc(C6CCC(=O)NC6=O)ccn5)CC4)CC3)cc21] |
| 445 | 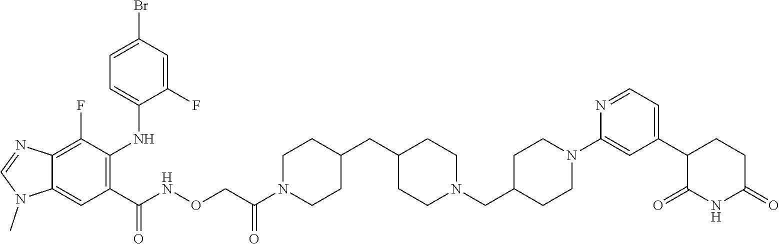 [SMILES: Cn1cnc2c(F)c(Nc3ccc(Br)cc3F)c(C(=O)NOCC(=O)N3CCC(CC4CCN(CC5CCN(c6cc(C7CCC(=O)NC7=O)ccn6)CC5)CC4)CC3)cc21] |
| 446 | 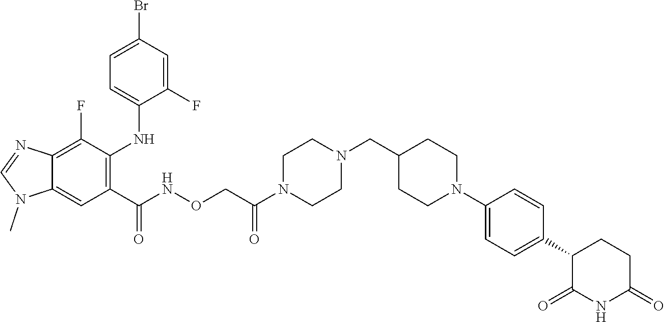 [SMILES: Cn1cnc2c(F)c(Nc3ccc(Br)cc3F)c(C(=O)NOCC(=O)N3CCN(CC4CCN(c5ccc([C@@H]6CCC(=O)NC6=O)cc5)CC4)CC3)cc21] |
| 447 | 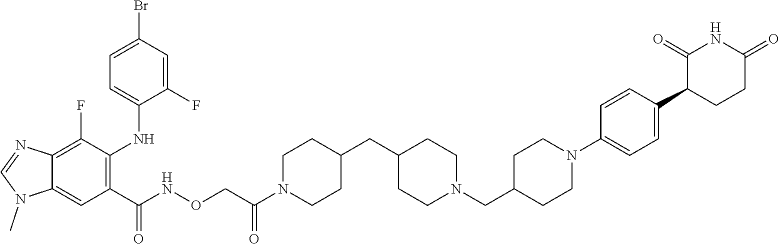 [SMILES: Cn1cnc2c(F)c(Nc3ccc(Br)cc3F)c(C(=O)NOCC(=O)N3CCC(CC4CCN(CC5CCN(c6ccc([C@@H]7CCC(=O)NC7=O)cc6)CC5)CC4)CC3)cc21] |
| 448 | 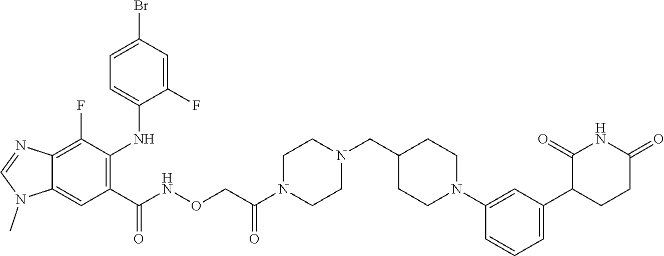 [SMILES: Cn1cnc2c(F)c(Nc3ccc(Br)cc3F)c(C(=O)NOCC(=O)N3CCN(CC4CCN(c5cccc(C6CCC(=O)NC6=O)c5)CC4)CC3)cc21] |
| 449 | 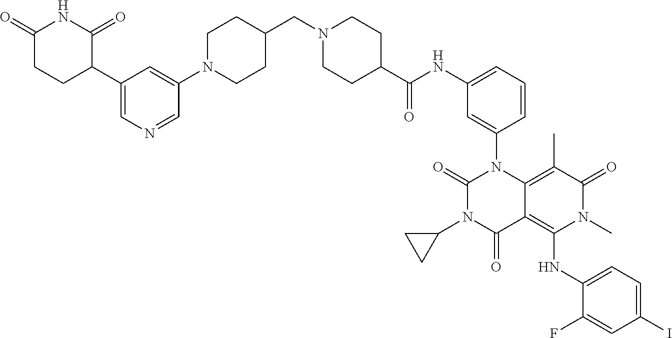 [SMILES: Cc1c(=O)n(C)c(Nc2ccc(I)cc2F)c2c(=O)n(C3CC3)c(=O)n(-c3cccc(NC(=O)C4CCN(CC5CCN(c6cncc(C7CCC(=O)NC7=O)c6)CC5)CC4)c3)c12] |
| 450 | 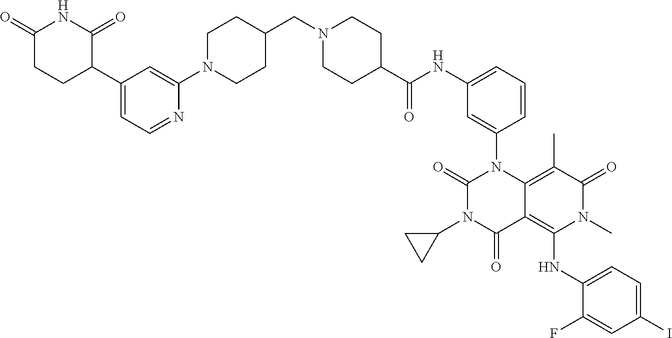 [SMILES: Cc1c(=O)n(C)c(Nc2ccc(I)cc2F)c2c(=O)n(C3CC3)c(=O)n(-c3cccc(NC(=O)C4CCN(CC5CCN(c6cc(C7CCC(=O)NC7=O)ccn6)CC5)CC4)c3)c12] |
| 451 | 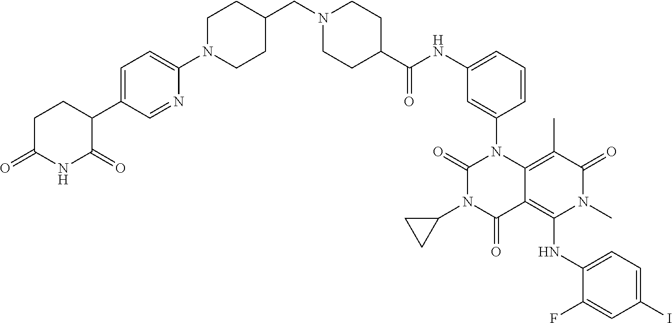 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc(C7CCC(=O)NC7=O)cn6)CC5)CC4)c3)c12] |
| 452 | 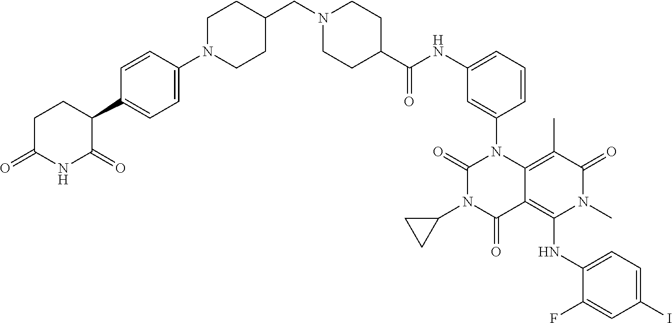 [SMILES: Cc1c(=O)n(C)c(Nc2ccc(I)cc2F)c2c(=O)n(C3CC3)c(=O)n(-c3cccc(NC(=O)C4CCN(CC5CCN(c6ccc([C@@H]7CCC(=O)NC7=O)cc6)CC5)CC4)c3)c12] |
| 453 | 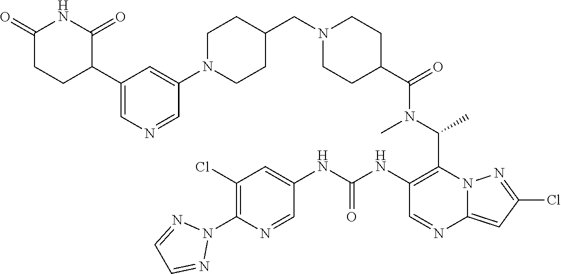 [SMILES: C[C@H](c1c(NC(=O)Nc2cnc(-n3nccn3)c(Cl)c2)cnc2cc(Cl)nn12)N(C)C(=O)C1CCN(CC2CCN(c3cncc(C4CCC(=O)NC4=O)c3)CC2)CC1] |
| 454 | 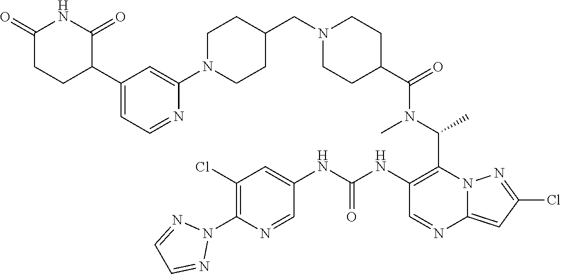 [SMILES: C[C@H](c1c(NC(=O)Nc2cnc(-n3nccn3)c(Cl)c2)cnc2cc(Cl)nn12)N(C)C(=O)C1CCN(CC2CCN(c3cc(C4CCC(=O)NC4=O)ccn3)CC2)CC1] |
| 455 | 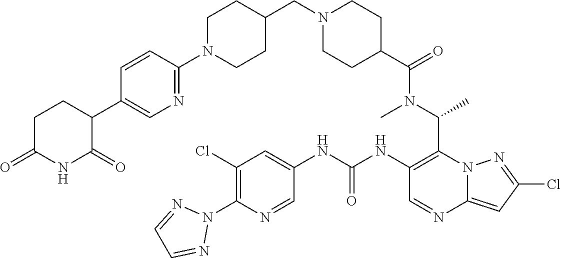 [SMILES: C[C@H](c1c(NC(=O)Nc2cnc(-n3nccn3)c(Cl)c2)cnc2cc(Cl)nn12)N(C)C(=O)C1CCN(CC2CCN(c3ccc(C4CCC(=O)NC4=O)cn3)CC2)CC1] |
| 456 | 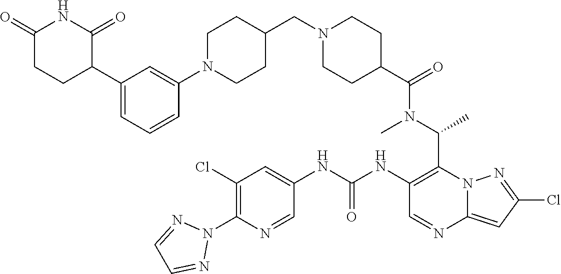 [SMILES: C[C@H](c1c(NC(=O)Nc2cnc(-n3nccn3)c(Cl)c2)cnc2cc(Cl)nn12)N(C)C(=O)C1CCN(CC2CCN(c3cccc(C4CCC(=O)NC4=O)c3)CC2)CC1] |
| 457 | 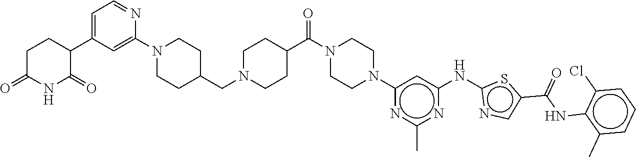 |
| 458 | 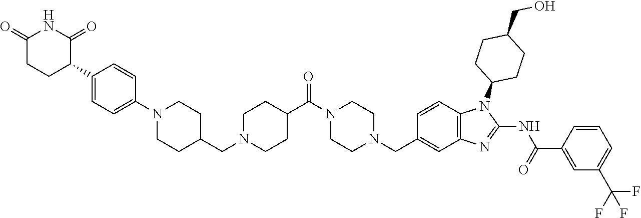 [SMILES: O=C1CC[C@@H](c2ccc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)cc2)C(=O)N1] |
| 459 | 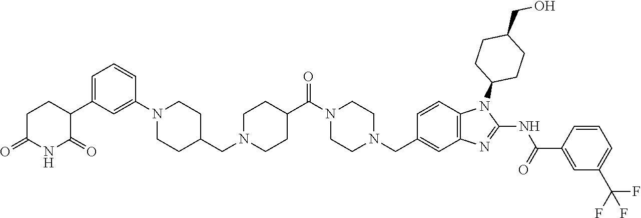 [SMILES: O=C1CCC(c2cccc(N3CCC(CN4CCC(C(=O)N5CCN(Cc6ccc7c(c6)nc(NC(=O)c6cccc(C(F)(F)F)c6)n7[C@H]6CC[C@@H](CO)CC6)CC5)CC4)CC3)c2)C(=O)N1] |
| 460 | 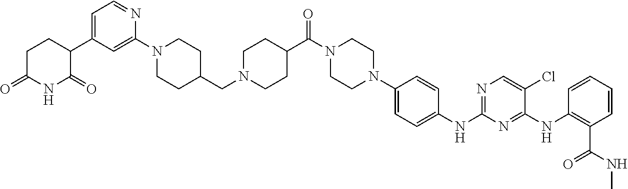 [SMILES: CNC(=O)c1ccccc1Nc1nc(Nc2ccc(N3CCN(C(=O)C4CCN(CC5CCN(c6cc(C7CCC(=O)NC7=O)ccn6)CC5)CC4)CC3)cc2)ncc1Cl] |
| 461 | 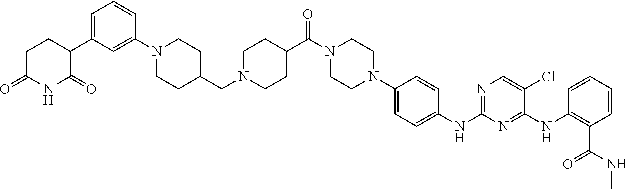 [SMILES: CNC(=O)c1ccccc1Nc1nc(Nc2ccc(N3CCN(C(=O)C4CCN(CC5CCN(c6cccc(C7CCC(=O)NC7=O)c6)CC5)CC4)CC3)cc2)ncc1Cl] |
| 462 | 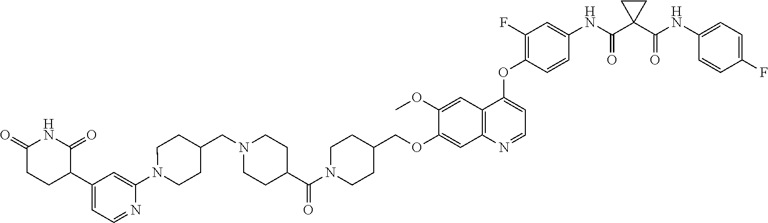 [SMILES: COc1cc2c(Oc3ccc(NC(=O)C4(C(=O)Nc5ccc(F)cc5)CC4)cc3F)ccnc2cc1OCC1CCN(C(=O)C2CCN(CC3CCN(c4cc(C5CCC(=O)NC5=O)ccn4)CC3)CC2)CC1] |
| 463 | 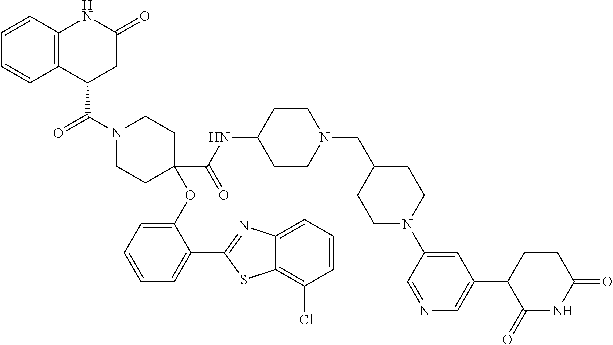 [SMILES: O=C1CCC(c2cncc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)c2)C(=O)N1] |
| 464 | 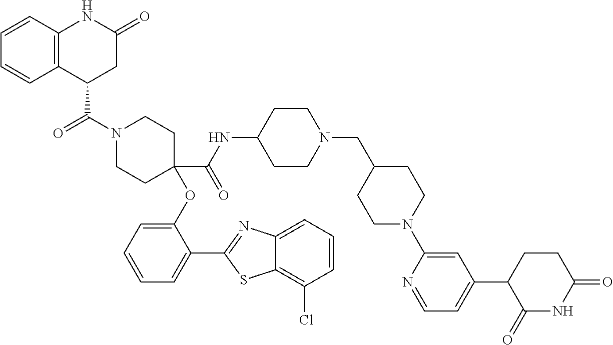 [SMILES: O=C1CCC(c2ccnc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)c2)C(=O)N1] |
| 465 | 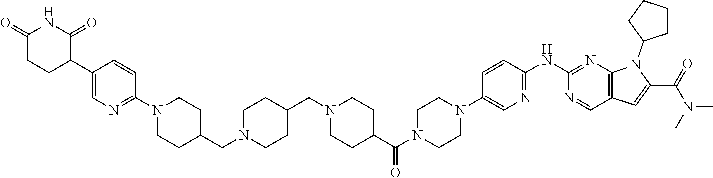 [SMILES: CN(C)C(=O)c1cc2cnc(Nc3ccc(N4CCN(C(=O)C5CCN(CC6CCN(CC7CCN(c8ccc(C9CCC(=O)NC9=O)cn8)CC7)CC6)CC5)CC4)cn3)nc2n1C1CCCC1] |
| 466 | 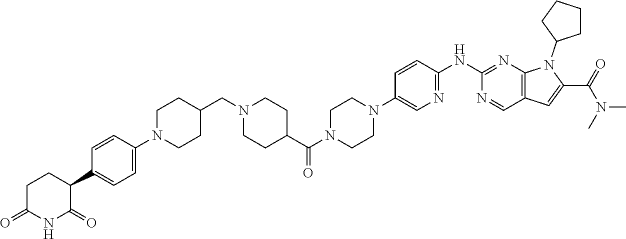 [SMILES: CN(C)C(=O)c1cc2cnc(Nc3ccc(N4CCN(C(=O)C5CCN(CC6CCN(c7ccc([C@@H]8CCC(=O)NC8=O)cc7)CC6)CC5)CC4)cn3)nc2n1C1CCCC1] |
| 467 | 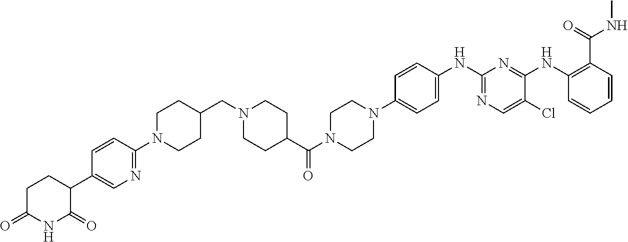 [SMILES: CNC(=O)c1ccccc1Nc1nc(Nc2ccc(N3CCN(C(=O)C4CCN(CC5CCN(c6ccc(C7CCC(=O)NC7=O)cn6)CC5)CC4)CC3)cc2)ncc1Cl] |
| 468 | 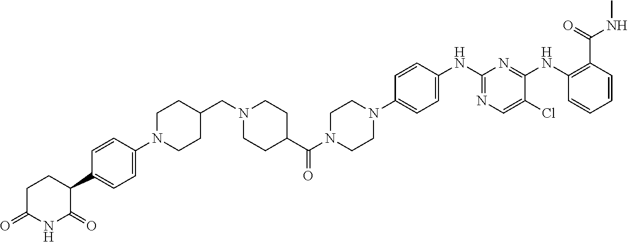 [SMILES: CNC(=O)c1ccccc1Nc1nc(Nc2ccc(N3CCN(C(=O)C4CCN(CC5CCN(c6ccc([C@@H]7CCC(=O)NC7=O)cc6)CC5)CC4)CC3)cc2)ncc1Cl] |
| 469 | 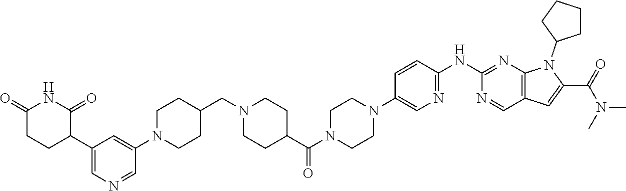 [SMILES: CN(C)C(=O)c1cc2cnc(Nc3ccc(N4CCN(C(=O)C5CCN(CC6CCN(c7cncc(C8CCC(=O)NC8=O)c7)CC6)CC5)CC4)cn3)nc2n1C1CCCC1] |
| 470 | 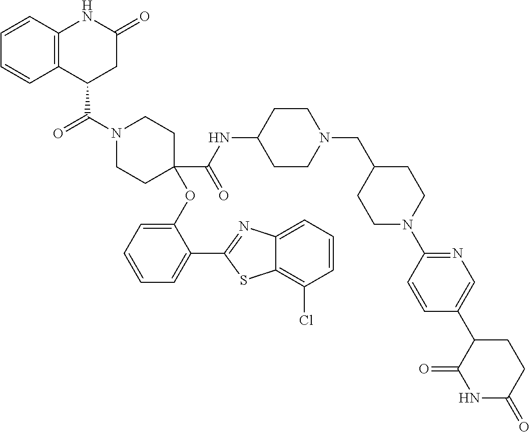 [SMILES: O=C1CCC(c2ccc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)nc2)C(=O)N1] |
| 471 | 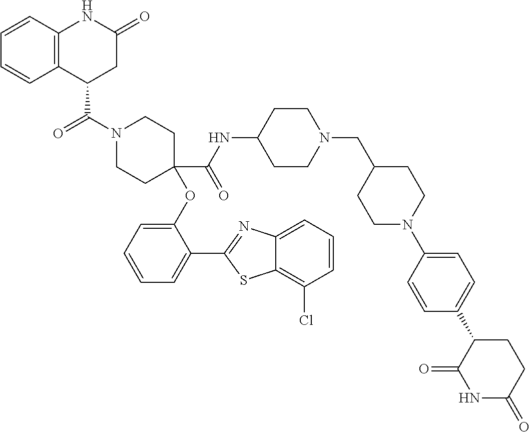 [SMILES: O=C1CC[C@@H](c2ccc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)cc2)C(=O)N1] |
| 472 | 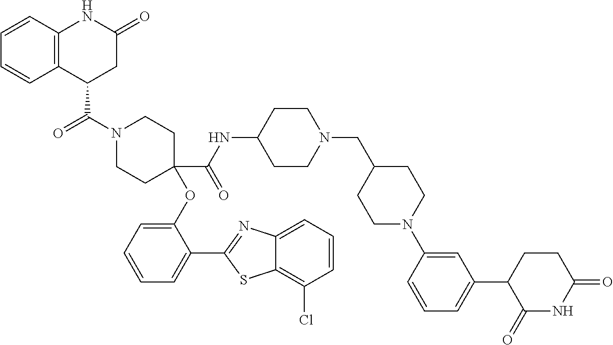 [SMILES: O=C1CCC(c2cccc(N3CCC(CN4CCC(NC(=O)C5(Oc6ccccc6-c6nc7cccc(Cl)c7s6)CCN(C(=O)[C@H]6CC(=O)Nc7ccccc76)CC5)CC4)CC3)c2)C(=O)N1] |
| 473 | 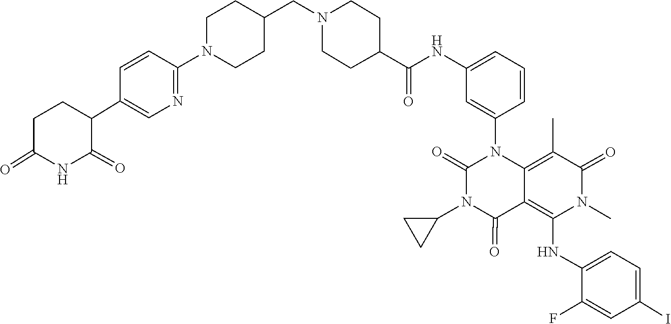 [SMILES: CCN(CCC(C)CN1CCC(C(=O)Nc2cccc(-n3c(=O)n(C4CC4)c(=O)c4c(Nc5ccc(I)cc5F)n(C)c(=O)c(C)c43)c2)CC1)c1ccc(C2CCC(=O)NC2=O)cn1] |
| 474 | 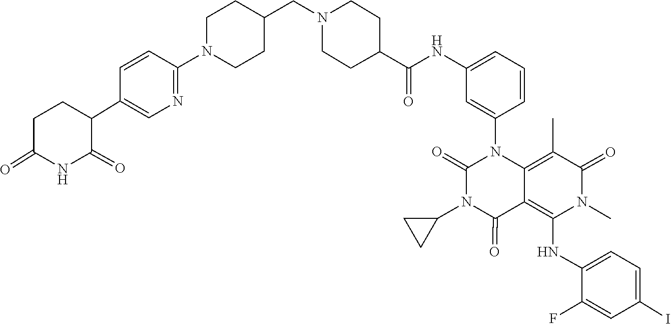 [SMILES: CCN(CCC(C)CN1CCC(C(=O)Nc2cccc(-n3c(=O)n(C4CC4)c(=O)c4c(Nc5ccc(I)cc5F)n(C)c(=O)c(C)c43)c2)CC1)c1ccc(C2CCC(=O)NC2=O)cn1] |
| 475 | 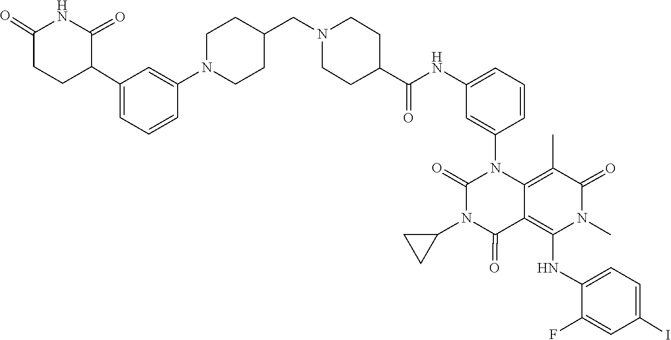 [SMILES: CCN(CCC(C)CN1CCC(C(=O)Nc2cccc(-n3c(=O)n(C4CC4)c(=O)c4c(Nc5ccc(I)cc5F)n(C)c(=O)c(C)c43)c2)CC1)c1cccc(C2CCC(=O)NC2=O)c1] |
| 476 | 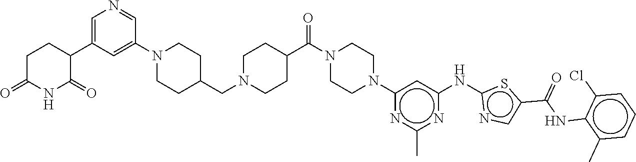 |
| 477 | 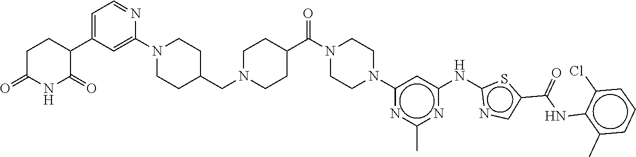 |
| 478 | 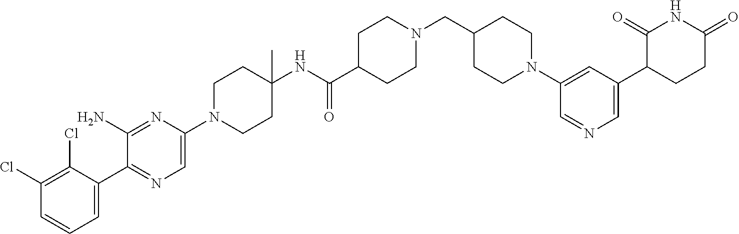 [SMILES: CC1(NC(=O)C2CCN(CC3CCN(c4cncc(C5CCC(=O)NC5=O)c4)CC3)CC2)CCN(c2cnc(-c3cccc(Cl)c3Cl)c(N)n2)CC1] |
| 479 | 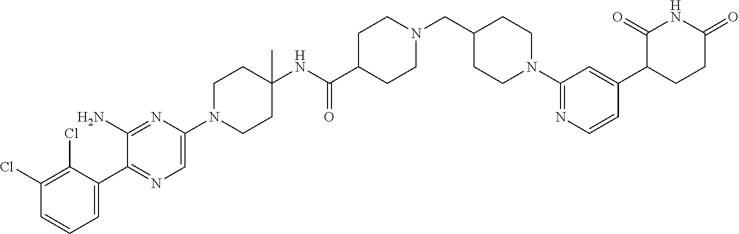 [SMILES: CC1(NC(=O)C2CCN(CC3CCN(c4cc(C5CCC(=O)NC5=O)ccn4)CC3)CC2)CCN(c2cnc(-c3cccc(Cl)c3Cl)c(N)n2)CC1] |
| 480 | 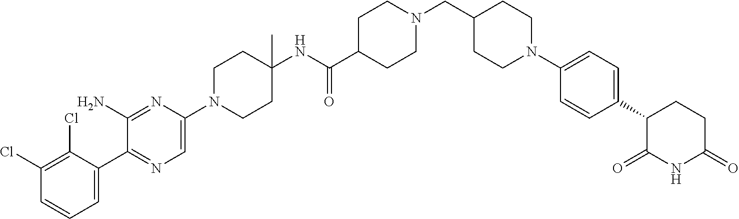 [SMILES: CC1(NC(=O)C2CCN(CC3CCN(c4ccc([C@@H]5CCC(=O)NC5=O)cc4)CC3)CC2)CCN(c2cnc(-c3cccc(Cl)c3Cl)c(N)n2)CC1] |
| 481 | 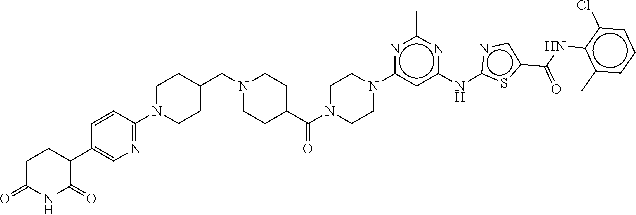 |
| 482 | 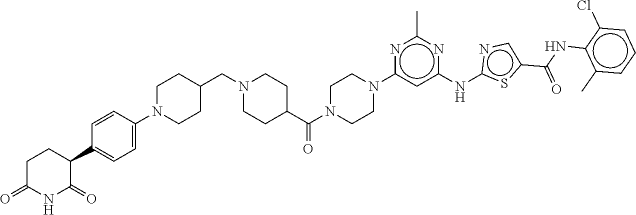 |
| 483 | 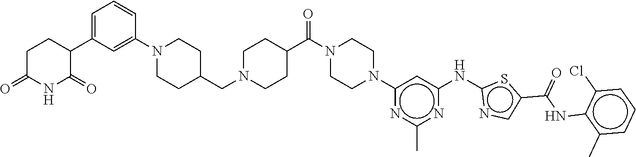 |
| 484 | 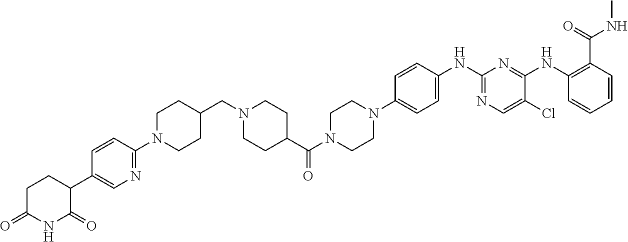 [SMILES: CNC(=O)c1ccccc1Nc1nc(Nc2ccc(N3CCN(C(=O)C4CCN(CC5CCN(c6ccc(C7CCC(=O)NC7=O)cn6)CC5)CC4)CC3)cc2)ncc1Cl] |
| 485 | 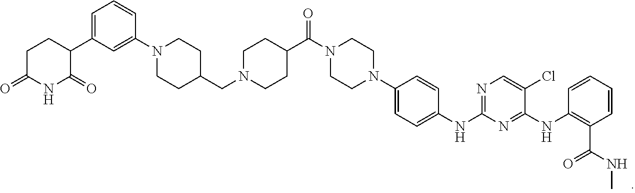 [SMILES: CNC(=O)c1ccccc1Nc1nc(Nc2ccc(N3CCN(C(=O)C4CCN(CC5CCN(c6cccc(C7CCC(=O)NC7=O)c6)CC5)CC4)CC3)cc2)ncc1Cl] |

248. The compound of claim 1, having the structure of compound 26 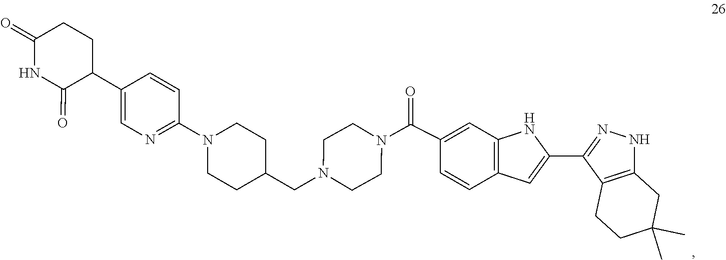 [SMILES: CC1(C)CCc2c(-c3cc4ccc(C(=O)N5CCN(CC6CCN(c7ccc(C8CCC(=O)NC8=O)cn7)CC6)CC5)cc4[nH]3)n[nH]c2C1]
or a stereoisomer and/or a pharmaceutically acceptable salt thereof.

249. A method of making a degrader compound capable of degrading a target protein in a cell comprising the steps of:
linking Y, a target binding moiety, which binds to a target protein, to a degron, directly or via a linker group L, to form the degrader compound.

250. A method of degrading a target protein in a cell, comprising the steps of making a degrader compound for the target protein according to claim 249, and contacting the target protein with the degrader compound.

251. The method of claim 249, wherein the target protein is selected from the group consisting of FLT3, SHP-2, GLD06, CDK4, CDK6, MALT1, BRAF, BCR-ABL, MEK, FGFR1, FGFR3, FGFR1 fusions, FGFR3 fusions, RET and fusions thereof, BCL-2, SARS-COV2 proteins, PI3K, PI3K Delta, MLL, Aurora A, KRAS, KRAS G12D, KRAS G12C, MDM2, MCL-1, PI3K alpha, e1F4e, KSR, and TRIM24.

252. The method of claim 249, wherein the Y target binding moieties have the capability to bind to a target protein selected from the group consisting Bruton's tyrosine kinase (BTK); tyrosine-protein kinase (ITK/TSK); bromodomain-containing protein 4 (BRD4); FMS-like tyrosine kinase 3 (FLT-3); Brg/Brahma-associated factors (BAF complex); induced myeloid leukemia cell differentiation protein (MCL-1); signal transducer and activator of transcription 3 (STAT3); barrier-to-autointegration factor (BAF); and BCR-ABL.

253. (canceled)

254. The method of claim 249 wherein the target binding moiety Y is selected from the group consisting of: 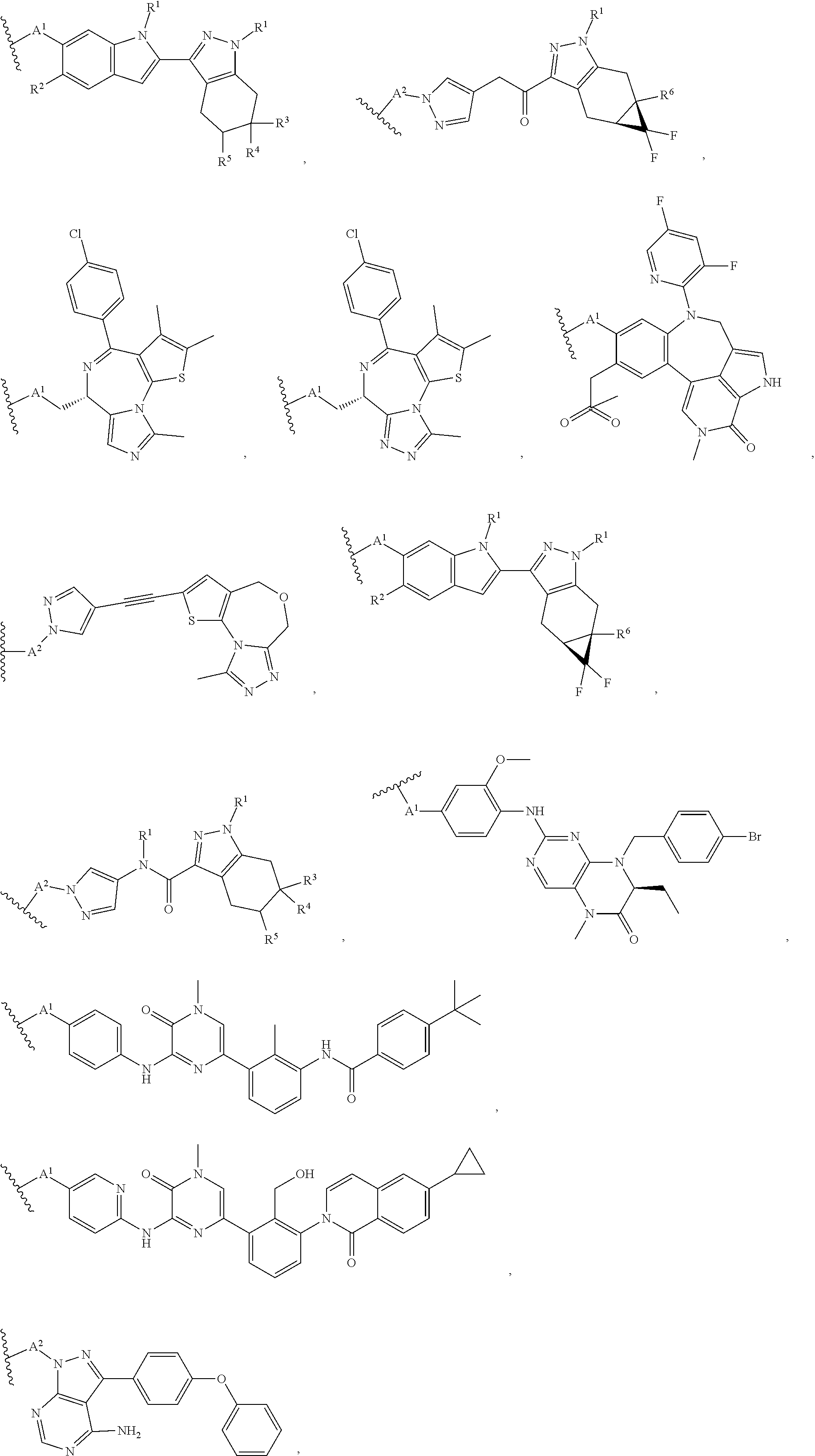 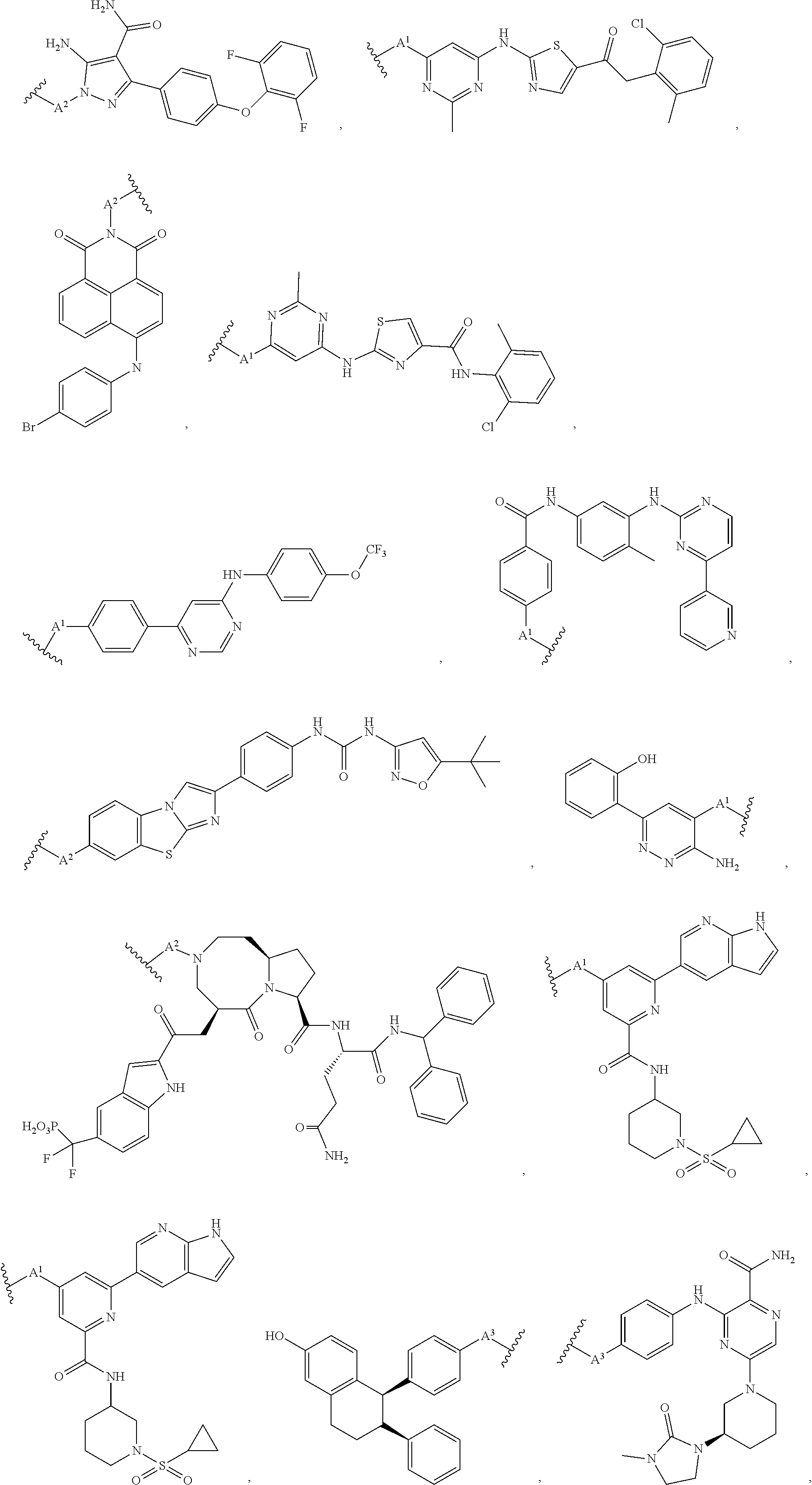 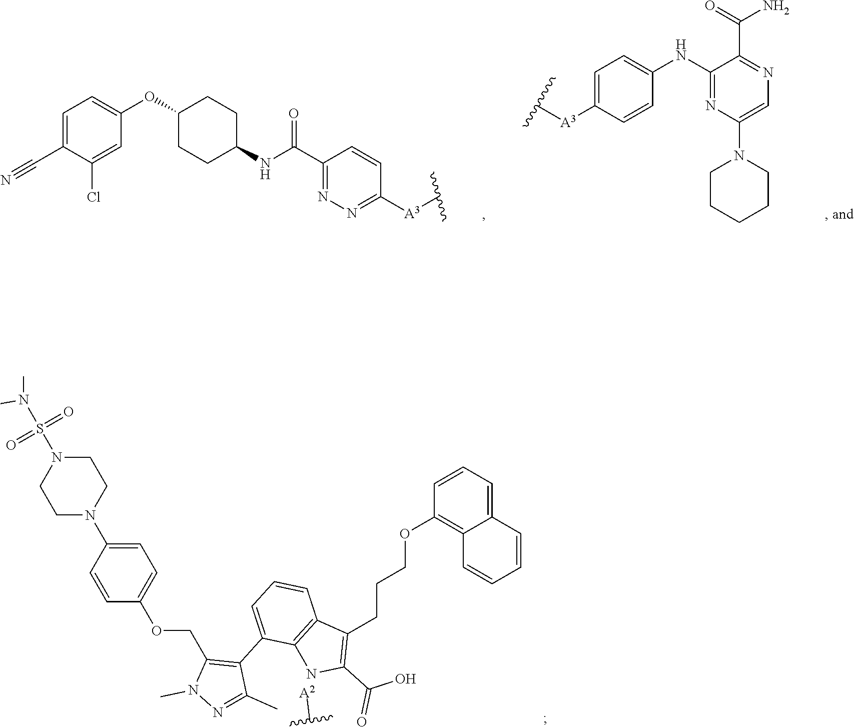
wherein:
R¹ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, or —C(O)heteroaryl;
R² is hydrogen, or halogen;
A¹ is absent, or —O—, —C(O)—, —C(O)—NH—, —C(O)—NH-A^{2a}-, —CH(A^{2a})-, —N(alkyl)-, -alkyl-;
A² is a or —C(H)(A^{2a})-, wherein A^{2a} is hydrogen, alkyl, aryl, heterocycle, or heteroaryl;
A³ is a bond;
R³ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, or —C(O)heteroaryl;
R⁴ is hydrogen, alkyl, alkenyl, alkynyl, cycloalkyl, heterocycloalkyl, aryl, alkaryl, aralkyl, haloalkyl, heteroaryl, cyano, —C(O)alkyl, —C(O)aryl, —C(O)heteroaryl, or an alkylene bound to R⁵ to form a substituted cycloalkyl;
R⁵ is hydrogen, or an alkylene bound to R⁴ to form the substituted cycloalkyl; wherein when R⁵ and R⁴ form the substituted cycloalkyl, then the cycloalkyl is substituted with one or more substituents selected from the group consisting of: alkyl, alkenyl, alkynyl, cycloalkyl, heterocycloalkyl, aryl, alkaryl, aralkyl, haloalkyl, heteroaryl, cyano, —C(O)alkyl, —C(O)aryl, —C(O)heteroaryl, and halogen; and
R⁶ is hydrogen, or alkyl.

255. The method of claim 249, wherein:
the target protein is FLT3, and the target binding moiety Y is selected from the group consisting of: 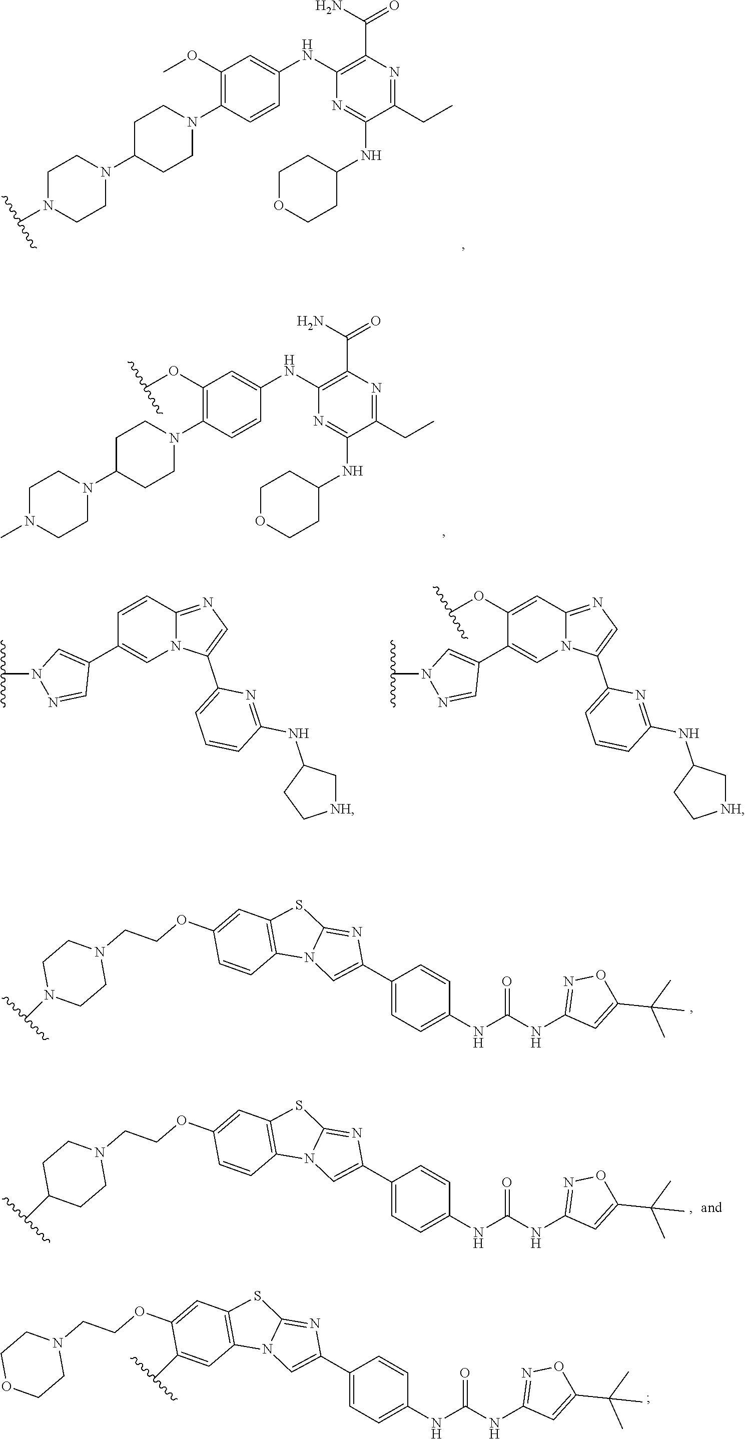
the target protein is B-RAF, and the target binding moiety Y is a residue of a compound selected from the group consisting of: 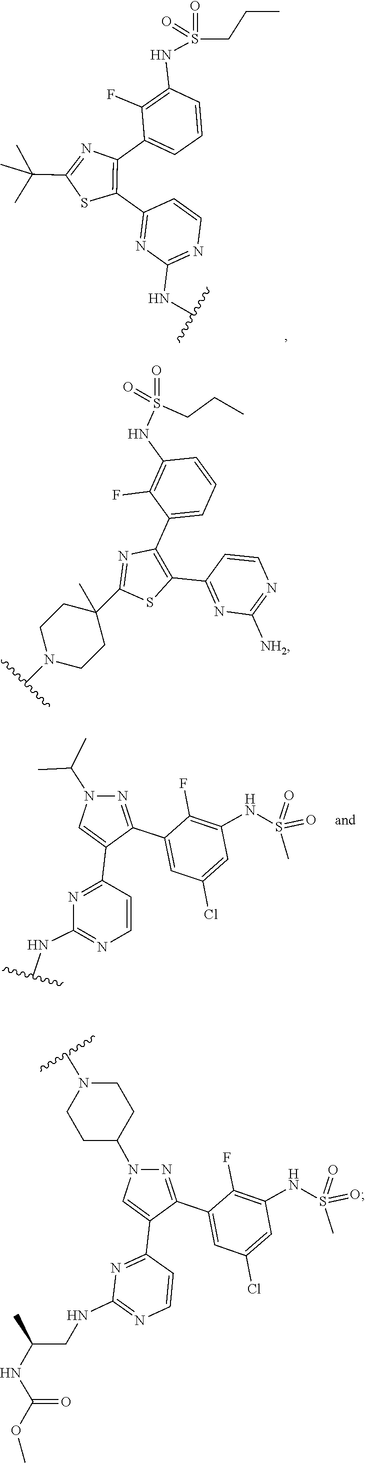
the target protein is B-RAF, and the target binding moiety Y is a residue of a compound selected from the group consisting of: 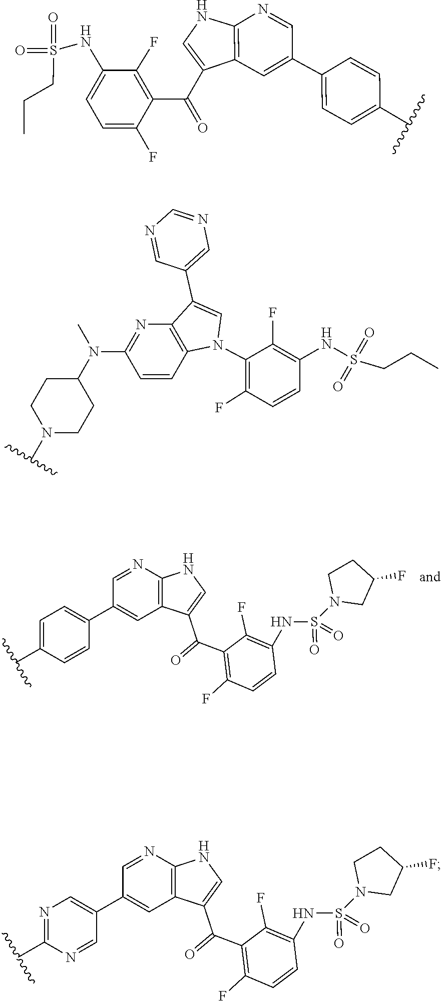
the target protein is CDK4 or CDK6, and the target binding moiety Y selected from the group consisting of: 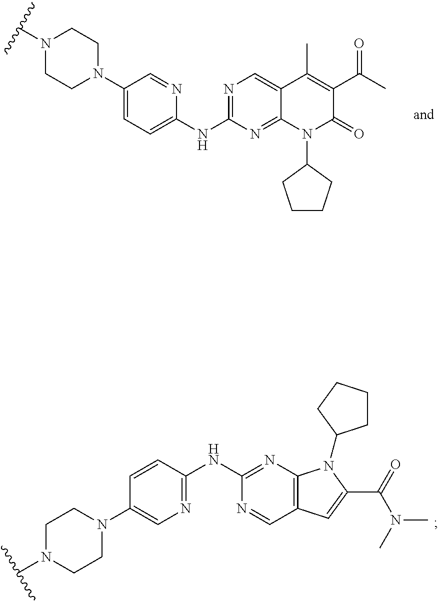 [SMILES: *N1CCN(c2ccc(Nc3ncc4c(C)c(C(C)=O)c(=O)n(C5CCCC5)c4n3)nc2)CC1 ; *N1CCN(c2ccc(Nc3ncc4cc(C(=O)N(C)C)n(C5CCCC5)c4n3)nc2)CC1]
the target protein is SHP-2, and the target binding moiety Y is a residue of a compound selected from the group consisting of: 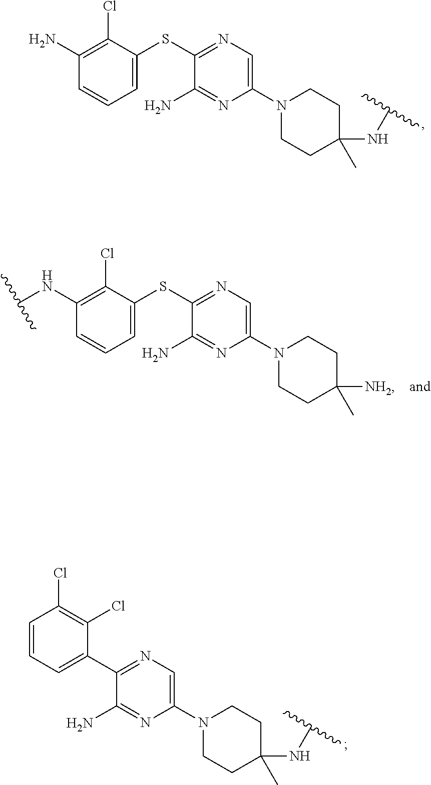 [SMILES: *NC1(C)CCN(c2cnc(Sc3cccc(N)c3Cl)c(N)n2)CC1 ; *Nc1cccc(Sc2ncc(N3CCC(C)(N)CC3)nc2N)c1Cl ; "and" ; *NC1(C)CCN(c2cnc(-c3cccc(Cl)c3Cl)c(N)n2)CC1]
the target protein is FGFR1 fusion and/or FGFR3 fusion, and the target binding moiety Y is according to: 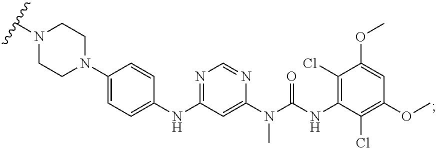 [SMILES: *N1CCN(c2ccc(Nc3cc(N(C)C(=O)Nc4c(Cl)c(OC)cc(OC)c4Cl)ncn3)cc2)CC1]
the target protein is MDM2, and the target binding moiety Y is: 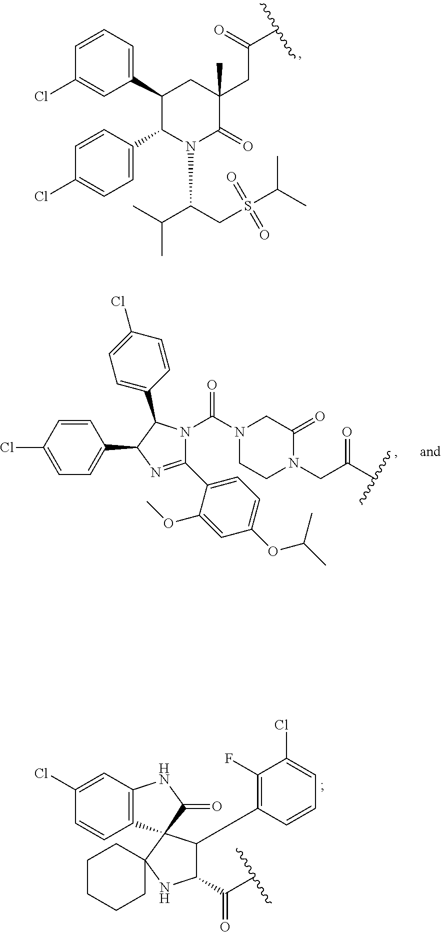 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O ; *C(=O)CN1CCN(C(=O)N2C(c3ccc(OC(C)C)cc3OC)=N[C@@H](c3ccc(Cl)cc3)[C@H]2c2ccc(Cl)cc2)CC1=O ; *C(=O)[C@@H]1NC2(CCCCC2)[C@@]2(C(=O)Nc3cc(Cl)ccc32)C1c1cccc(Cl)c1F]
the target protein is TRIM24, and the target binding moiety Y is: 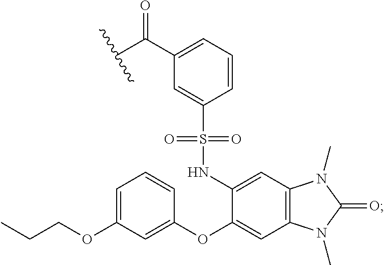 [SMILES: *C(=O)c1cccc(S(=O)(=O)Nc2cc3c(cc2Oc2cccc(OCCC)c2)n(C)c(=O)n3C)c1]
the target is a SARS-COV2 protein, and the target binding moiety Y is selected from the group consisting of: 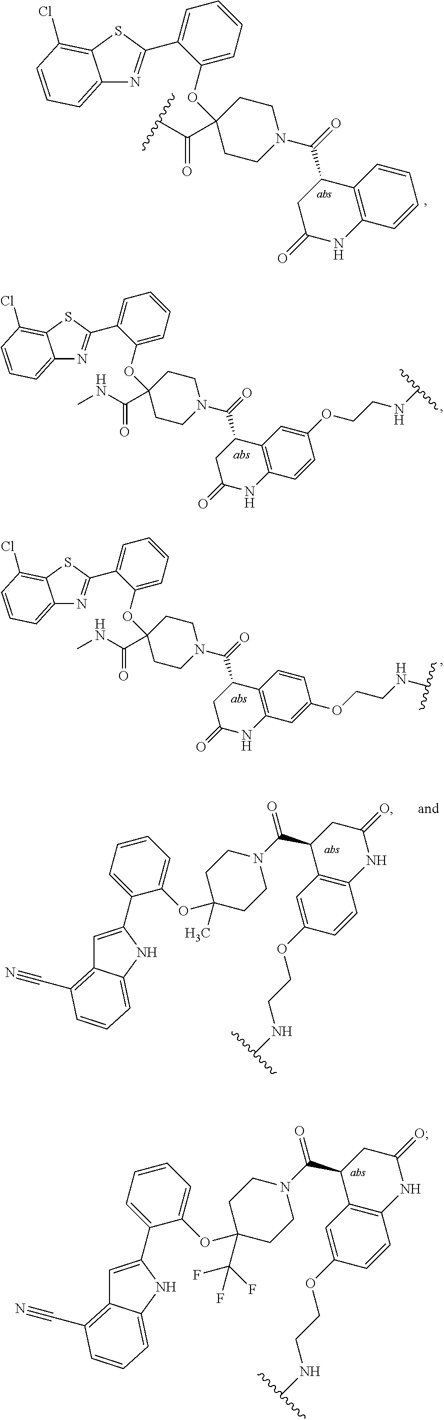 [SMILES: *C(=O)C1(Oc2ccccc2-c2nc3cccc(Cl)c3s2)CCN(C(=O)[C@H]2CC(=O)Nc3ccccc32)CC1 ; *NCCOc1ccc2c(c1)[C@@H](C(=O)N1CCC(Oc3ccccc3-c3nc4cccc(Cl)c4s3)(C(=O)NC)CC1)CC(=O)N2 ; *NCCOc1ccc2c(c1)NC(=O)C[C@@H]2C(=O)N1CCC(Oc2ccccc2-c2nc3cccc(Cl)c3s2)(C(=O)NC)CC1 ; *NCCOc1ccc2c(c1)[C@@H](C(=O)N1CCC(C)(Oc3ccccc3-c3cc4c(C#N)cccc4[nH]3)CC1)CC(=O)N2 ; "and" ; *NCCOc1ccc2c(c1)[C@@H](C(=O)N1CCC(Oc3ccccc3-c3cc4c(C#N)cccc4[nH]3)(C(F)(F)F)CC1)CC(=O)N2]
the target protein is PI3K delta, and the target binding moiety Y is: 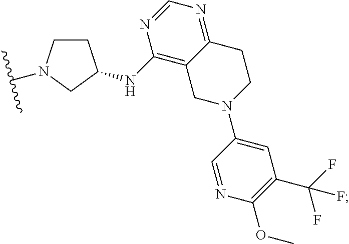 [SMILES: *N1CC[C@H](Nc2ncnc3c2CN(c2cnc(OC)c(C(F)(F)F)c2)CC3)C1]
the target protein is MEK, and the target binding moiety Y is selected from the group consisting of: 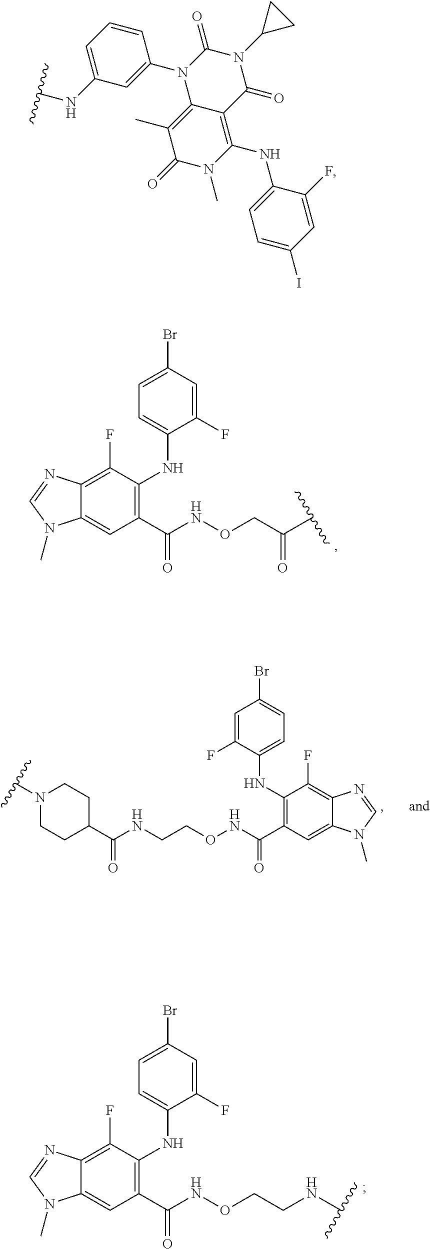 [SMILES: *Nc1cccc(-n2c(=O)n(C3CC3)c(=O)c3c(Nc4ccc(I)cc4F)n(C)c(=O)c(C)c32)c1 ; *C(=O)CONC(=O)c1cc2c(ncn2C)c(F)c1Nc1ccc(Br)cc1F ; *N1CCC(C(=O)NCCONC(=O)c2cc3c(ncn3C)c(F)c2Nc2ccc(Br)cc2F)CC1 ; *NCCONC(=O)c1cc2c(ncn2C)c(F)c1Nc1ccc(Br)cc1F]
the target protein is BCR-ABL, and the target binding moiety Y is selected from the group consisting of: 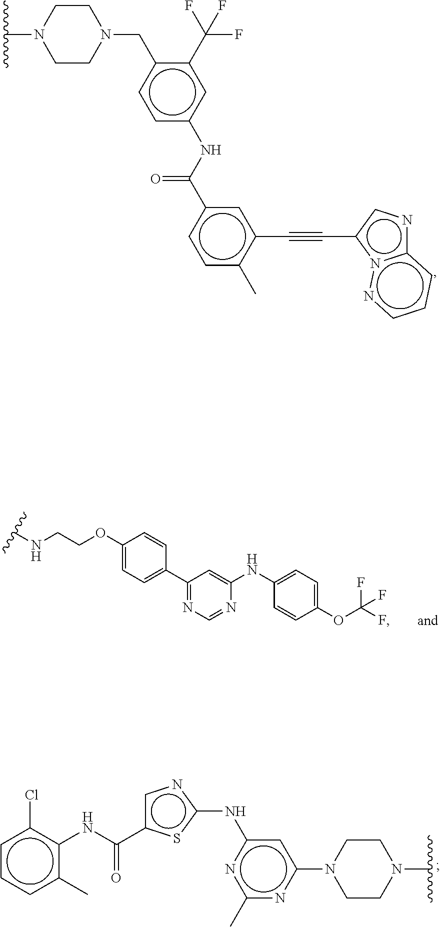
the target protein is MLL, and the target binding moiety Y is: 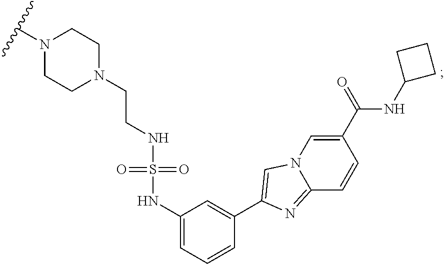 [SMILES: *N1CCN(CCNS(=O)(=O)Nc2cccc(-c3cn4cc(C(=O)NC5CCC5)ccc4n3)c2)CC1]
the target protein is MALT1, and the target binding moiety Y is selected from the group consisting of: 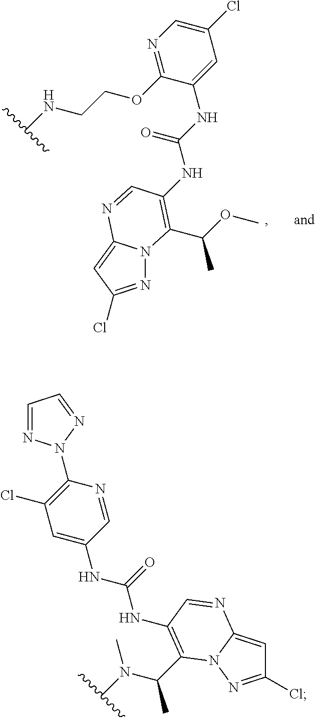 [SMILES: *NCCOc1ncc(Cl)cc1NC(=O)Nc1cnc2cc(Cl)nn2c1[C@H](C)OC ; *N(C)[C@H](C)c1c(NC(=O)Nc2cnc(-n3nccn3)c(Cl)c2)cnc2cc(Cl)nn12]
the target protein is IRAK1, and the target binding moiety Y is: 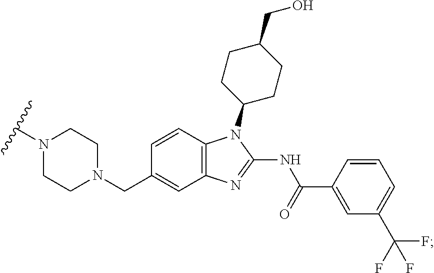 [SMILES: *N1CCN(Cc2ccc3c(c2)nc(NC(=O)c2cccc(C(F)(F)F)c2)n3[C@H]2CC[C@@H](CO)CC2)CC1]
the target protein is one or more kinases, and the target binding moiety Y is: 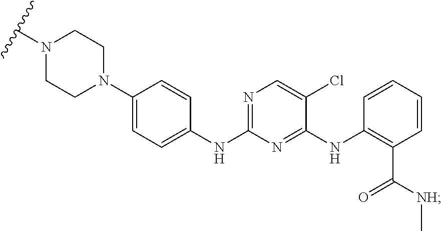 [SMILES: *N1CCN(c2ccc(Nc3ncc(Cl)c(Nc4ccccc4C(=O)NC)n3)cc2)CC1]
or
the target protein is one or more kinases, and the target binding moiety Y is:

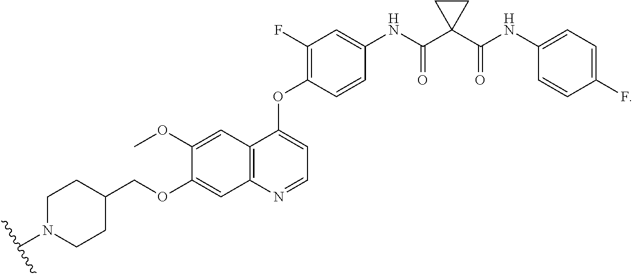 [SMILES: *N1CCC(COc2cc3nccc(Oc4ccc(NC(=O)C5(C(=O)Nc6ccc(F)cc6)CC5)cc4F)c3cc2OC)CC1]

256.-271. (canceled)

272. The method of claim 249, wherein the degron binds an E3 ubiquitin ligase.

273. The method of claim 249, wherein the degron binds cereblon.

274. (canceled)

275. The method of claim 249, wherein the degron is according to 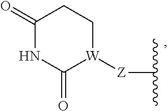
wherein:
W is —CH;
Z is selected from the group consisting of: 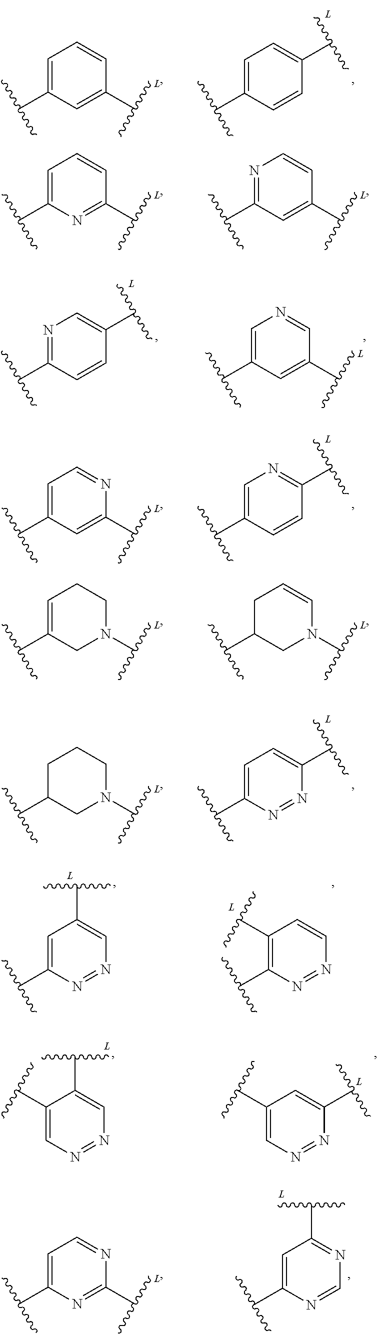 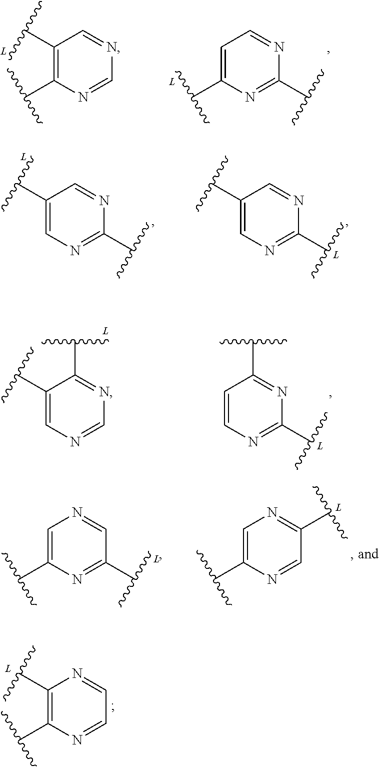
and
L is the linker group.

276. (canceled)

277. The method of claim 249, wherein linker L comprises 3 to 40 carbon atoms, comprising one or more acyclic and/or cyclic moieties, wherein one or more carbons atoms can be replaced optionally by a heteroatom selected from O, N, S, and P, or wherein the linker group comprises 1, 2, 3, 4, 5, 6, 7, 8, 9, or 10 ethylene glycol units; wherein the linker group is optionally substituted.

278. The method of claim 249, wherein L, the linker group is according to -L¹-L²-L³-L⁴-L⁵-L⁶-L⁷-,
wherein:
-L¹- is absent, —N(R²¹)—, C(R²²)—, C₁₋₈ alkylene, C₂₋₈ alkynylene, Q¹, or Q²;
each -L²-, -L³-, -L⁴-, and -L⁵- is independently, absent, —N(R²¹)—, C(R²²)—, —C(O)—, —O—, —(CH₂—CH₂—O)₁₋₈—, C₁₋₈ alkylene, C₂₋₈ alkynylene, Q¹, Q², or Q³;
each -L⁶- and -L⁷-, is independently, absent, —N(R²¹)—, —C(R²²)—, —C(O)—, —C(O)—N(R²¹)—, —N(R²¹)—C(O), or —C(R²²)—C(O)—N(R²¹);
each Q¹ is a three- to seven-membered heterocycloalkylene comprising at least one nitrogen;
each Q² is a five- to thirteen-membered spiro bicyclic heterocycloalkylene comprising at least one nitrogen;
each Q³ is a three- to six-membered cycloalkylene;
R²¹ is H, or methyl; and
R²² is H, methyl, aryl, or heteroaryl.

279. The method of claim 249, wherein the linker group L is selected from: 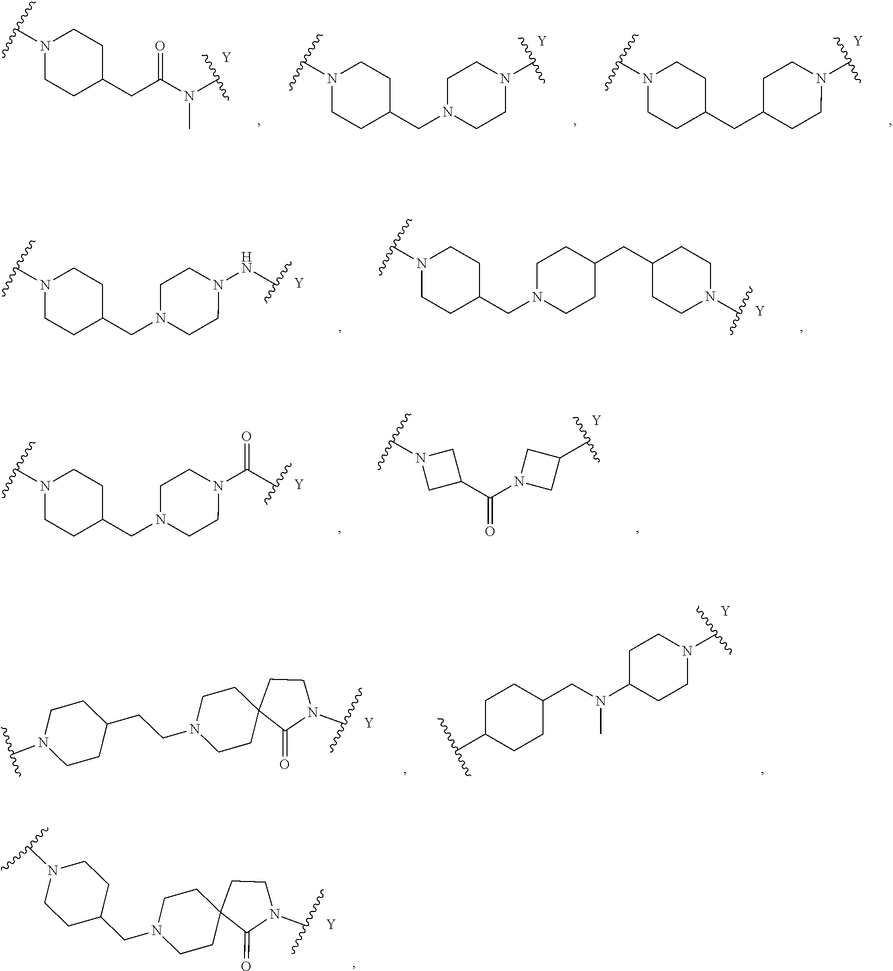 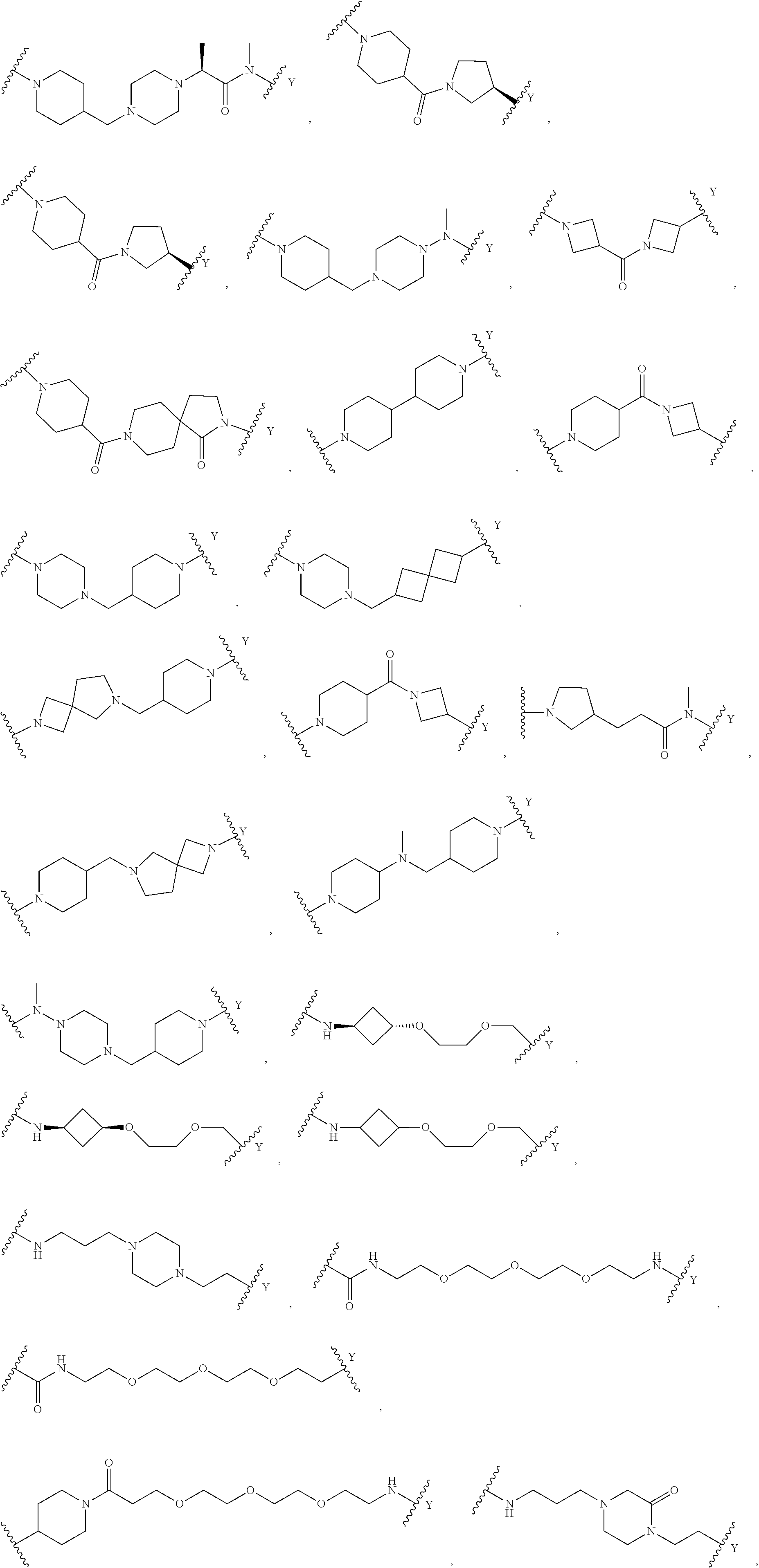 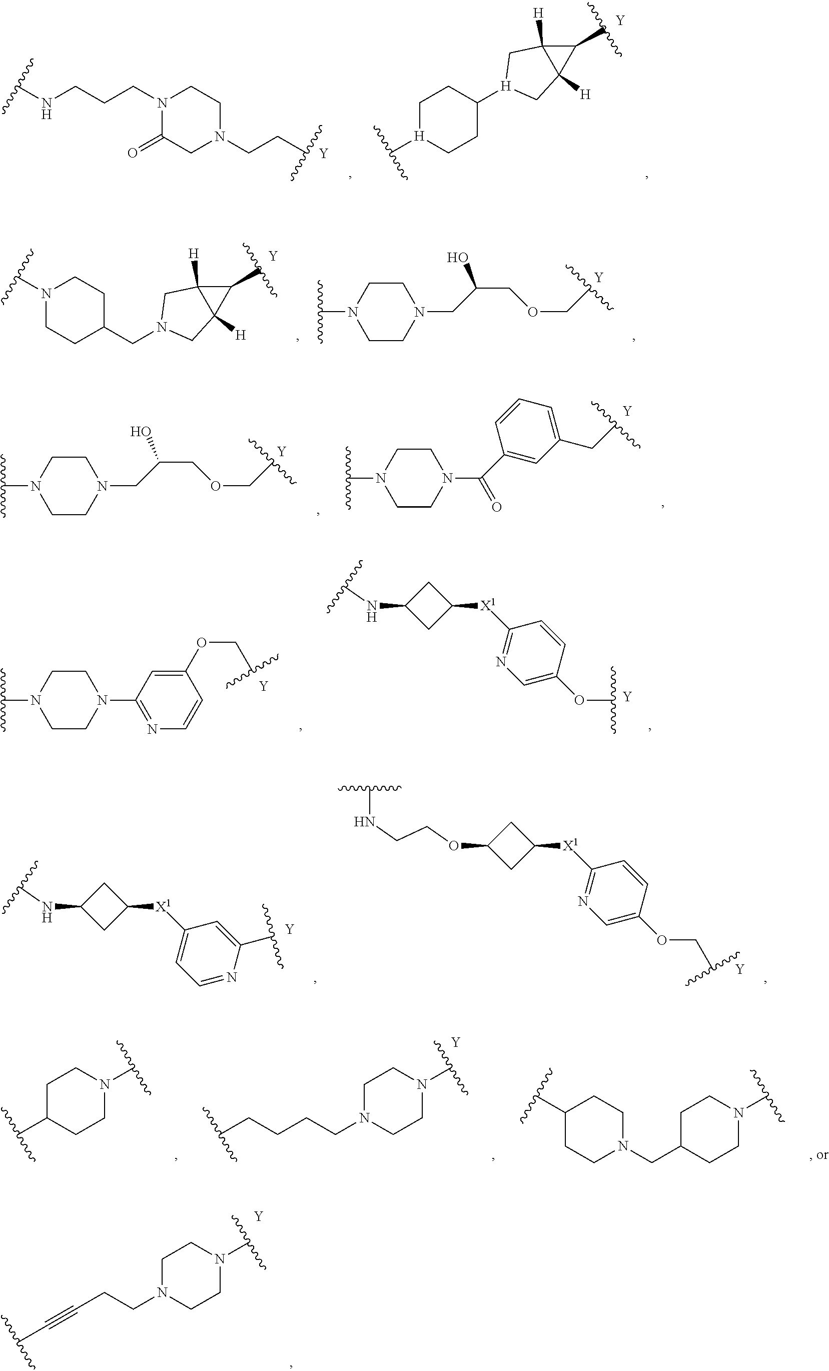
wherein, [symbol] designates attachment to Y.

280. A compound produced by the method of claim 249.

281. A pharmaceutical composition comprising the compound of claim 1, or a stereoisomer and/or a pharmaceutically acceptable salt thereof, and one or more pharmaceutically acceptable carriers, excipients, or diluents.

282. A method of treating a disease or disorder mediated by a target protein in a patient in need thereof, comprising administering to the patient a compound or pharmaceutically acceptable salt of a compound of claim 1.

283.-315. (canceled)

316. A method of treating a disease or disorder mediated by a target protein in a patient in need thereof, comprising administering to the patient a pharmaceutical composition of claim 281.
